SEQUENCE LISTING (157 sequences)

SEQ ID NO: 1 - RNA, 20 nt, synthetic construct
  source 1..20: mol_type = other RNA
  ncRNA 1..20: ncRNA_class = guide_RNA
tctctccatgtgcagtagga

SEQ ID NO: 2 - RNA, 20 nt, synthetic construct
  source 1..20: mol_type = other RNA
  ncRNA 1..20: ncRNA_class = guide_RNA
ctggatgtcggaggagtacg

SEQ ID NO: 3 - RNA, 20 nt, synthetic construct
  source 1..20: mol_type = other RNA
  ncRNA 1..20: ncRNA_class = guide_RNA
gtctccggaaactcgaggtg

SEQ ID NO: 4 - RNA, 20 nt, synthetic construct
  source 1..20: mol_type = other RNA
  ncRNA 1..20: ncRNA_class = guide_RNA
acagtgtagacttgattgac

SEQ ID NO: 5 - RNA, 20 nt, synthetic construct
  source 1..20: mol_type = other RNA
  ncRNA 1..20: ncRNA_class = guide_RNA
cgtgagtaaacctgaatctt

SEQ ID NO: 6 - protein, 21 aa, synthetic construct
MALPVTALLLPLALLLHAARP

SEQ ID NO: 7 - protein, 20 aa, synthetic construct
METDTLLLWVLLLWVPGSTG

SEQ ID NO: 8 - protein, 22 aa, synthetic construct
MLLLVTSLLLCELPHPAFLLIP

SEQ ID NO: 9 - protein, 45 aa, synthetic construct
TTTPAPRPPTPAPTIASQPLSLRPEACRPAAGGAVHTRGLDFACD

SEQ ID NO: 10 - protein, 39 aa, synthetic construct
IEVMYPPPYLDNEKSNGTIIHVKGKHLCPSPLFPGPSKP

SEQ ID NO: 11 - protein, 12 aa, synthetic construct
ESKYGPPCPPCP

SEQ ID NO: 12 - protein, 12 aa, synthetic construct
ESKYGPPCPSCP

SEQ ID NO: 13 - protein, 507 aa, synthetic construct
MEPAGPAPGRLGPLLCLLLAASCAWSGVAGEEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIYIVVGVVCTLLVALLMAALYLVRIRQKKAQGSTSSTRLHEPEKNAREITQVQSLDTNDITYADLNLPKGKKPAPQAAEPNNHTEYASIQTSPQPASEDTLTYADLDMVHLNRTPKQPAPKPEPSFSEYASVQVPRK

SEQ ID NO: 14 - protein, 330 aa, Homo sapiens [sequence omitted]

SEQ ID NO: 15 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 16 - protein, 327 aa, Homo sapiens
ASFKGPSVFPLVPCSRSTSESTAALGCLVKDYFPEPVTVSWNSCALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVRVLTVLHQDWLNGKEYKCKVSNKGLPSSIEKTISKAKGQPREPQVYTLPPSQEEMTKNQVSLTCLVKGFYPSDIAVEWESNGQPEDNYKTTPPVLDSDGSFFLYSRLTVDKSRWQEGNVFSCSVMHEALHNHYTQKSLSLSPGK

SEQ ID NO: 17 - protein, 345 aa, synthetic construct
EEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 347 aa, synthetic construct
EEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 19 - protein, 245 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 20 - protein, 107 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 21 - protein, 6 aa, synthetic construct
QDISKY

SEQ ID NO: 22 - skipped

SEQ ID NO: 23 - protein, 9 aa, synthetic construct
QQGNTLPYT

SEQ ID NO: 24 - protein, 18 aa, synthetic construct
GSTSGSGKPGSGEGSTKG

SEQ ID NO: 25 - protein, 120 aa, synthetic construct
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 26 - protein, 8 aa, synthetic construct
GVSLPDYG

SEQ ID NO: 27 - protein, 7 aa, synthetic construct
IWGSETT

SEQ ID NO: 28 - protein, 14 aa, synthetic construct
AKHYYYGGSYAMDY

SEQ ID NO: 29 - protein, 242 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 30 - protein, 15 aa, synthetic construct
GGGGSGGGGSGGGGS

SEQ ID NO: 31 - DNA, 1458 bp, synthetic construct
  source 1..1458: mol_type = other DNA
atggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 32 - protein, 486 aa, synthetic construct
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 33 - DNA, 1383 bp, synthetic construct
  source 1..1383: mol_type = other DNA
atgctgctgctggtgaccagcctgctgctgtgcgagctgccccaccccgcctttctgctgatccccgacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcccagccggtttagcggcagcggctccggcaccgactacagcctgaccatctccaacctggaacaggaagatatcgccacctacttttgccagcagggcaacacactgccctacacctttggcggcggaacaaagctggaaatcaccggcagcacctccggcagcggcaagcctggcagcggcgagggcagcaccaagggcgaggtgaagctgcaggaaagcggccctggcctggtggcccccagccagagcctgagcgtgacctgcaccgtgagcggcgtgagcctgcccgactacggcgtgagctggatccggcagccccccaggaagggcctggaatggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagcgaatctaagtacggaccgccctgccccccttgccctatgttctgggtgctggtggtggtcggaggcgtgctggcctgctacagcctgctggtcaccgtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaagg

SEQ ID NO: 34 - protein, 461 aa, synthetic construct
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 35 - DNA, 1467 bp, synthetic construct
  source 1..1467: mol_type = other DNA
atgcttctcctggtgacaagccttctgctctgtgagttaccacacccagcattcctcctgatcccagacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataacaggctccacctctggatccggcaagcccggatctggcgagggatccaccaagggcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctcagcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttgggggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 36 - protein, 489 aa, synthetic construct
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 37 - protein, 246 aa, synthetic construct
DIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGSTSGSGKPGSGEGSTKGEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSS

SEQ ID NO: 38 - protein, 106 aa, synthetic construct
DIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIK

SEQ ID NO: 39 - protein, 10 aa, synthetic construct
RASSSVNYMD

SEQ ID NO: 40 - protein, 7 aa, synthetic construct
ATSNLAS

SEQ ID NO: 41 - protein, 9 aa, synthetic construct
QQWSFNPPT

SEQ ID NO: 42 - protein, 122 aa, synthetic construct
EVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSS

SEQ ID NO: 43 - protein, 5 aa, synthetic construct
SYNMH

SEQ ID NO: 44 - protein, 17 aa, synthetic construct
AIYPGNGDTSYNQKFKG

SEQ ID NO: 45 - protein, 246 aa, synthetic construct
QVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSGGGGSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIK

SEQ ID NO: 46 - protein, 124 aa, synthetic construct
QVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSS

SEQ ID NO: 47 - protein, 10 aa, synthetic construct
GDSVSSNSAA

SEQ ID NO: 48 - protein, 9 aa, synthetic construct
TYYRSKWYN

SEQ ID NO: 49 - protein, 14 aa, synthetic construct
AREVTGDLEDAFDI

SEQ ID NO: 50 - protein, 107 aa, synthetic construct
DIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIK

SEQ ID NO: 51 - protein, 6 aa, synthetic construct
QTIWSY

SEQ ID NO: 52 - skipped

SEQ ID NO: 53 - protein, 9 aa, synthetic construct
QQSYSIPQT

SEQ ID NO: 54 - protein, 246 aa, synthetic construct
QVQLQQSGPGMVKPSQTLSLTCAISGDSVSSNSVAWNWIRQSPSRGLEWLGRTYYRSTWYNDYAVSMKSRITINPDTNKNQFSLQLNSVTPEDTAVYYCAREVTGDLEDAFDIWGQGTMVTVSSGGGGSGGGGSGGGGSDIQMIQSPSSLSASVGDRVTITCRASQTIWSYLNWYRQRPGEAPNLLIYAASSLQSGVPSRFSGRGSGTDFTLTISSLQAEDFATYYCQQSYSIPQTFGQGTKLEIK

SEQ ID NO: 55 - protein, 124 aa, synthetic construct
QVQLQQSGPGMVKPSQTLSLTCAISGDSVSSNSVAWNWIRQSPSRGLEWLGRTYYRSTWYNDYAVSMKSRITINPDTNKNQFSLQLNSVTPEDTAVYYCAREVTGDLEDAFDIWGQGTMVTVSS

SEQ ID NO: 56 - protein, 10 aa, synthetic construct
GDSVSSNSVA

SEQ ID NO: 57 - protein, 9 aa, synthetic construct
TYYRSTWYN

SEQ ID NO: 58 - protein, 14 aa, synthetic construct
AREVTGDLEDAFDI

SEQ ID NO: 59 - protein, 107 aa, synthetic construct
DIQMIQSPSSLSASVGDRVTITCRASQTIWSYLNWYRQRPGEAPNLLIYAASSLQSGVPSRFSGRGSGTDFTLTISSLQAEDFATYYCQQSYSIPQTFGQGTKLEIK

SEQ ID NO: 60 - protein, 6 aa, synthetic construct
QTIWSY

SEQ ID NO: 61 - skipped

SEQ ID NO: 62 - protein, 9 aa, synthetic construct
QQSYSIPQT

SEQ ID NO: 63 - protein, 246 aa, synthetic construct
DIVLTQSPASLAMSLGKRATISCRASESVSVIGAHLIHWYQQKPGQPPKLLIYLASNLETGVPARFSGSGSGTDFTLTIDPVEEDDVAIYSCLQSRIFPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSS

SEQ ID NO: 64 - protein, 111 aa, synthetic construct
DIVLTQSPASLAMSLGKRATISCRASESVSVIGAHLIHWYQQKPGQPPKLLIYLASNLETGVPARFSGSGSGTDFTLTIDPVEEDDVAIYSCLQSRIFPRTFGGGTKLEIK

SEQ ID NO: 65 - protein, 15 aa, synthetic construct
RASESVSVIGAHLIH

SEQ ID NO: 66 - protein, 7 aa, synthetic construct
LASNLET

SEQ ID NO: 67 - protein, 9 aa, synthetic construct
LQSRIFPRT

SEQ ID NO: 68 - protein, 117 aa, synthetic construct
QIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSS

SEQ ID NO: 69 - protein, 5 aa, synthetic construct
DYSIN

SEQ ID NO: 70 - protein, 17 aa, synthetic construct
WINTETREPAYAYDFRG

SEQ ID NO: 71 - protein, 8 aa, synthetic construct
DYSYAMDY

SEQ ID NO: 72 - protein, 246 aa, synthetic construct
DIVLTQSPPSLAMSLGKRATISCRASESVTILGSHLIYWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFRHYSMNWVKQAPGKGLKWMGRINTESGVPIYADDFKGRFAFSVETSASTAYLVINNLKDEDTASYFCSNDYLYSLDFWGQGTALTVSS

SEQ ID NO: 73 - protein, 111 aa, synthetic construct
DIVLTQSPPSLAMSLGKRATISCRASESVTILGSHLIYWYQQKPGQPPTLLIQLASNVQTGVPARFSGSGSRTDFTLTIDPVEEDDVAVYYCLQSRTIPRTFGGGTKLEIK

SEQ ID NO: 74 - protein, 15 aa, synthetic construct
RASESVTILGSHLIY

SEQ ID NO: 75 - protein, 7 aa, synthetic construct
LASNVQT

SEQ ID NO: 76 - protein, 9 aa, synthetic construct
LQSRTIPRT

SEQ ID NO: 77 - protein, 117 aa, synthetic construct
QIQLVQSGPELKKPGETVKISCKASGYTFRHYSMNWVKQAPGKGLKWMGRINTESGVPIYADDFKGRFAFSVETSASTAYLVINNLKDEDTASYFCSNDYLYSLDFWGQGTALTVSS

SEQ ID NO: 78 - protein, 5 aa, synthetic construct
HYSMN

SEQ ID NO: 79 - protein, 17 aa, synthetic construct
RINTESGVPIYADDFKG

SEQ ID NO: 80 - protein, 8 aa, synthetic construct
DYLYSLDF

SEQ ID NO: 81 - protein, 121 aa, synthetic construct
EVQLLESGGGLVQPGGSLRLSCAASGFTFSSYAMSWVRQAPGKGLEWVSSISGSGDYIYYADSVKGRFTISRDISKNTLYLQMNSLRAEDTAVYYCAKEGTGANSSLADYRGQGTLVTVSS

SEQ ID NO: 82 - protein, 8 aa, synthetic construct
GFTFSSYA

SEQ ID NO: 83 - protein, 8 aa, synthetic construct
ISGSGDYI

SEQ ID NO: 84 - protein, 14 aa, synthetic construct
AKEGTGANSSLADY

SEQ ID NO: 85 - protein, 236 aa, synthetic construct
QVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIK

SEQ ID NO: 86 - protein, 69 aa, synthetic construct
TTTPAPRPPTPAPTIASQPLSLRPEACRPAAGGAVHTRGLDFACDIYIWAPLAGTCGVLLLSLVITLYC

SEQ ID NO: 87 - protein, 65 aa, synthetic construct
VMYPPPYLDNEKSNGTIIHVKGKHLCPSPLFPGPSKPFWVLVVVGGVLACYSLLVTVAFIIFWVR

SEQ ID NO: 88 - protein, 40 aa, synthetic construct
SKRSRLLHSDYMNMTPRRPGPTRKHYQPYAPPRDFAAYRS

SEQ ID NO: 89 - protein, 84 aa, synthetic construct
FVPVFLPAKPTTTPAPRPPTPAPTIASQPLSLRPEACRPAAGGAVHTRGLDFACDIYIWAPLAGTCGVLLLSLVITLYCNHRNR

SEQ ID NO: 90 - protein, 47 aa, synthetic construct
RFSVVKRGRKKLLYIFKQPFMRPVQTTQEEDGCSCRFPEEEEGGCEL

SEQ ID NO: 91 - protein, 484 aa, synthetic construct
MLLLVTSLLLCELPHPAFLLIPQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 92 - protein, 480 aa, synthetic construct
MLLLVTSLLLCELPHPAFLLIPQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 93 - protein, 544 aa, synthetic construct
MLLLVTSLLLCELPHPAFLLIPQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKAAAFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 94 - RNA, 20 nt, synthetic construct
  source 1..20: mol_type = other RNA
  ncRNA 1..20: ncRNA_class = guide_RNA
cagcctcctggttatggtac

SEQ ID NO: 95 - RNA, 18 nt, synthetic construct
  source 1..18: mol_type = other RNA
  ncRNA 1..18: ncRNA_class = guide_RNA
gctacgtatccatcgtga

SEQ ID NO: 96 - RNA, 19 nt, synthetic construct
  source 1..19: mol_type = other RNA
  ncRNA 1..19: ncRNA_class = guide_RNA
gtacacatccaggtgacgc

SEQ ID NO: 97 - RNA, 18 nt, synthetic construct
  source 1..18: mol_type = other RNA
  ncRNA 1..18: ncRNA_class = guide_RNA
gcaggctgatgctacggg

SEQ ID NO: 98 - RNA, 20 nt, synthetic construct
  source 1..20: mol_type = other RNA
  ncRNA 1..20: ncRNA_class = guide_RNA
tcaccaagcccgcgaccaat

SEQ ID NO: 99 - RNA, 20 nt, synthetic construct
  source 1..20: mol_type = other RNA
  ncRNA 1..20: ncRNA_class = guide_RNA
tcaccaagcccgcgaccaat

SEQ ID NO: 100 - RNA, 20 nt, synthetic construct
  source 1..20: mol_type = other RNA
  ncRNA 1..20: ncRNA_class = guide_RNA
atcaccaagcccgcgaccaa

SEQ ID NO: 101 - RNA, 20 nt, synthetic construct
  source 1..20: mol_type = other RNA
  ncRNA 1..20: ncRNA_class = guide_RNA
cggtgcggcgcaggccctat

SEQ ID NO: 102 - RNA, 20 nt, synthetic construct
  source 1..20: mol_type = other RNA
  ncRNA 1..20: ncRNA_class = guide_RNA
gctttggtcccattggtcgc

SEQ ID NO: 103 - RNA, 20 nt, synthetic construct
  source 1..20: mol_type = other RNA
  ncRNA 1..20: ncRNA_class = guide_RNA
gcccgcaggacgcacccata

SEQ ID NO: 104 - RNA, 20 nt, synthetic construct
  source 1..20: mol_type = other RNA
  ncRNA 1..20: ncRNA_class = guide_RNA
gtgcatccagcgcttcgcac

SEQ ID NO: 105 - RNA, 20 nt, synthetic construct
  source 1..20: mol_type = other RNA
  ncRNA 1..20: ncRNA_class = guide_RNA
cagctacgtatccatcgtga

SEQ ID NO: 106 - RNA, 20 nt, synthetic construct
  source 1..20: mol_type = other RNA
  ncRNA 1..20: ncRNA_class = guide_RNA
ggacaaaggcgcaagtcgag

SEQ ID NO: 107 - protein, 13 aa, synthetic construct
SNYYGSSYWFFDV

SEQ ID NO: 108-112 - skipped (5 entries)

SEQ ID NO: 113 - protein, 42 aa, synthetic construct
AAAIEVMYPPPYLDNEKSNGTIIHVKGKHLCPSPLFPGPSKP

SEQ ID NO: 114 - protein, 28 aa, synthetic construct
MFWVLVVVGGVLACYSLLVTVAFIIFWV

SEQ ID NO: 115 - protein, 112 aa, synthetic construct
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 116 - DNA, 1467 bp, synthetic construct
  source 1..1467: mol_type = other DNA
atggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggctccacctctggatccggcaagcccggatctggcgagggatccaccaagggcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 117 - protein, 489 aa, synthetic construct
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 118 - protein, 243 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQKYDLLTFGGGTKVEIKGSTSGSGKPGSGEGSTKGQLQLQESGPGLVKPSETLSLTCTVSGGSISSSSYYWGWIRQPPGKGLEWIGSISYSGSTYYNPSLKSRVTISVDTSKNQFSLKLSSVTAADTAVYYCARDRGDTILDVWGQGTMVTVSS

SEQ ID NO: 119 - protein, 106 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQKYDLLTFGGGTKVEIK

SEQ ID NO: 120 - protein, 6 aa, synthetic construct
QSISSY

SEQ ID NO: 121 - skipped

SEQ ID NO: 122 - protein, 8 aa, synthetic construct
QQKYDLLT

SEQ ID NO: 123 - protein, 119 aa, synthetic construct
QLQLQESGPGLVKPSETLSLTCTVSGGSISSSSYYWGWIRQPPGKGLEWIGSISYSGSTYYNPSLKSRVTISVDTSKNQFSLKLSSVTAADTAVYYCARDRGDTILDVWGQGTMVTVSS

SEQ ID NO: 124 - protein, 10 aa, synthetic construct
GGSISSSSYY

SEQ ID NO: 125 - protein, 7 aa, synthetic construct
ISYSGST

SEQ ID NO: 126 - protein, 11 aa, synthetic construct
ARDRGDTILDV

SEQ ID NO: 127 - DNA, 1503 bp, synthetic construct
  source 1..1503: mol_type = other DNA
atggccttaccagtgaccgccttgctcctgccgctggccttgctgctccacgccgccaggccggacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcagcaaaaatacgacctcctcacttttggcggagggaccaaggttgagatcaaaggcagcaccagcggctccggcaagcctggctctggcgagggcagcacaaagggacagctgcagctgcaggagtcgggcccaggactggtgaagccttcggagaccctgtccctcacctgcactgtctctggtggctccatcagcagtagtagttactactggggctggatccgccagcccccagggaaggggctggagtggattgggagtatctcctatagtgggagcacctactacaacccgtccctcaagagtcgagtcaccatatccgtagacacgtccaagaaccagttctccctgaagctgagttctgtgaccgccgcagacacggcggtgtactactgcgccagagatcgtggagacaccatactagacgtatggggtcagggtacaatggtcaccgtcagctcattcgtgcccgtgttcctgcccgccaaacctaccaccacccctgcccctagacctcccaccccagccccaacaatcgccagccagcctctgtctctgcggcccgaagcctgtagacctgctgccggcggagccgtgcacaccagaggcctggacttcgcctgcgacatctacatctgggcccctctggccggcacctgtggcgtgctgctgctgagcctggtgatcaccctgtactgcaaccaccggaacaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcagatccgccgacgcccctgcctaccagcagggacagaaccagctgtacaacgagctgaacctgggcagacgggaagagtacgacgtgctggacaagcggagaggccgggaccccgagatgggcggaaagcccagacggaagaacccccaggaaggcctgtataacgaactgcagaaagacaagatggccgaggcctacagcgagatcggcatgaagggcgagcggaggcgcggcaagggccacgatggcctgtaccagggcctgagcaccgccaccaaggacacctacgacgccctgcacatgcaggccctgccccccaga

SEQ ID NO: 128 - protein, 501 aa, synthetic construct
MALPVTALLLPLALLLHAARPDIQMTQSPSSLSASVGDRVTITCRASQSISSYLNWYQQKPGKAPKLLIYAASSLQSGVPSRFSGSGSGTDFTLTISSLQPEDFATYYCQQKYDLLTFGGGTKVEIKGSTSGSGKPGSGEGSTKGQLQLQESGPGLVKPSETLSLTCTVSGGSISSSSYYWGWIRQPPGKGLEWIGSISYSGSTYYNPSLKSRVTISVDTSKNQFSLKLSSVTAADTAVYYCARDRGDTILDVWGQGTMVTVSSFVPVFLPAKPTTTPAPRPPTPAPTIASQPLSLRPEACRPAAGGAVHTRGLDFACDIYIWAPLAGTCGVLLLSLVITLYCNHRNKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 129 - RNA, 20 nt, synthetic construct
  source 1..20: mol_type = other RNA
  ncRNA 1..20: ncRNA_class = guide_RNA
gatattggcataagcctccc

SEQ ID NO: 130 - RNA, 20 nt, synthetic construct
  source 1..20: mol_type = other RNA
  ncRNA 1..20: ncRNA_class = guide_RNA
agagtctctcagctggtaca

SEQ ID NO: 131 - skipped

SEQ ID NO: 132 - RNA, 12 nt, synthetic construct
  source 1..12: mol_type = other RNA
  ncRNA 1..12: ncRNA_class = guide_RNA
gttttagagcta

SEQ ID NO: 133 - skipped

SEQ ID NO: 134 - RNA, 63 nt, synthetic construct
  source 1..63: mol_type = other RNA
  ncRNA 1..63: ncRNA_class = guide_RNA
tagcaagttaaaataaggctagtccgttatcaacttgaaaaagtggcaccgagtcggtgcttt

SEQ ID NO: 135 - RNA, 99 nt, synthetic construct
  source 1..99: mol_type = other RNA
  ncRNA 1..99: ncRNA_class = guide_RNA
nnnnnnnnnnnnnnnnnnnngttttagagctagaaatagcaagttaaaataaggctagtccgttatcaacttgaaaaagtggcaccgagtcggtgcttt

SEQ ID NO: 136 - protein, 305 aa, Homo sapiens
QLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 137 - protein, 323 aa, Homo sapiens
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 138-139 - skipped (2 entries)

SEQ ID NO: 140 - protein, 229 aa, synthetic construct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 141 - protein, 24 aa, synthetic construct
IYIWAPLAGTCGVLLLSLVITLYC

SEQ ID NO: 142 - protein, 27 aa, synthetic construct
FWVLVVVGGVLACYSLLVTVAFIIFWV

SEQ ID NO: 143 - protein, 42 aa, synthetic construct
KRGRKKLLYIFKQPFMRPVQTTQEEDGCSCRFPEEEEGGCEL

SEQ ID NO: 144 - protein, 41 aa, synthetic construct
RSKRSRLLHSDYMNMTPRRPGPTRKHYQPYAPPRDFAAYRS

SEQ ID NO: 145 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 146 - RNA, 66 nt, synthetic construct
  source 1..66: mol_type = other RNA
  ncRNA 1..66: ncRNA_class = guide_RNA
tagcaagttaaaataaggctagtccgttatcaacttgaaaaagtggcaccgagtcggtgctttttt

SEQ ID NO: 147 - RNA, 17 nt, synthetic construct
  source 1..17: mol_type = other RNA
  ncRNA 1..17: ncRNA_class = guide_RNA
gtttsagagctatgctg

SEQ ID NO: 148 - RNA, 71 nt, synthetic construct
  source 1..71: mol_type = other RNA
  ncRNA 1..71: ncRNA_class = guide_RNA
cagcatagcaagttsaaataaggctagtccgttatcaacttgaaaaagtggcaccgagtcggtgctttttt

SEQ ID NO: 149 - RNA, 14 nt, synthetic construct
  source 1..14: mol_type = other RNA
  ncRNA 1..14: ncRNA_class = guide_RNA
gttttagtactctg

SEQ ID NO: 150 - RNA, 64 nt, synthetic construct
  source 1..64: mol_type = other RNA
  ncRNA 1..64: ncRNA_class = guide_RNA
cagaatctactaaaacaaggcaaaatgccgtgtttatctcgtcaacttgttggcgagatttttt

SEQ ID NO: 151 - RNA, 88 nt, synthetic construct
  source 1..88: mol_type = other RNA
  ncRNA 1..88: ncRNA_class = guide_RNA
gtcgtctataggacggcgaggacaacgggaagtgccaatgtgctctttccaagagcaaacaccccgttggcttcaagatgaccgctcg

SEQ ID NO: 152 - RNA, 23 nt, synthetic construct
  source 1..23: mol_type = other RNA
  ncRNA 1..23: ncRNA_class = guide_RNA
cgagcggtctgagaagtggcact

SEQ ID NO: 153 - protein, 5 aa, synthetic construct
  REGION >5: note = The entire sequence of amino acids 1-5 can be repeated one or more times
GGGGS

SEQ ID NO: 154 - protein, 5 aa, synthetic construct
LEWIG

SEQ ID NO: 155 - protein, 6 aa, synthetic construct
  REGION >6: note = The entire sequence of amino acids 1-6 can be repeated one to ten times
SGGGGS

SEQ ID NO: 156 - protein, 7 aa, synthetic construct
  REGION >7: note = The entire sequence of amino acids 1-7 can be repeated one to ten times
GGGGSSS

SEQ ID NO: 157 - protein, 4 aa, synthetic construct
  REGION >4: note = The entire sequence of amino acids 1-4 can be repeated one to six times
GGGS

(19) United States
(12) Patent Application Publication
(10) Pub. No.: US 2025/0302953 A1
(43) Pub. Date: Oct. 2, 2025

(54) METHODS OF TREATING PATIENTS EXHIBITING A PRIOR FAILED THERAPY WITH HYPOIMMUNOGENIC CELLS

(71) Applicant: Sana Biotechnology, Inc., Seattle, WA (US)

(72) Inventors: Terry James Fry, Denver, CO (US); Hosein Kouros-Mehr, Los Angeles, CA (US); Adam James Johnson, Shoreline, WA (US)

(21) Appl. No.: 18/838,120

(22) PCT Filed: Feb. 14, 2023
(86) PCT No.: PCT/US23/13070

§ 371 Date: Aug. 13, 2024

Related U.S. Application Data
(60) Provisional application No. 63/310,086, filed on Feb. 14, 2022.

(51) Int. Cl. A61K 40/11; A61K 40/31; A61K 40/42; A61P 35/00

(52) U.S. Cl. CPC A61K 40/11; A61K 40/31; A61K 40/4211; A61K 40/4212; A61P 35/00; A61K 2239/48

(57) ABSTRACT

Disclosed herein are engineered cells comprising one or more CARs directed to a first therapeutic target and one or more CARs directed to a second therapeutic target, as well as methods of using such engineered cells. Also provided herein are methods of treating a disease or disorder in a patient that has previously been administered one or more targeted therapies, the method comprising administering a population of engineered CAR-T cells to the patient. In some embodiments, one or more targeted therapies comprised administration of a first therapeutic agent, wherein the first therapeutic agent is directed to a first therapeutic target. In some embodiments, engineered CAR-T cells of the population comprise one or more chimeric antigen receptors (CARs), wherein at least one CAR encoded by the one or more exogenous polynucleotides is directed to a second therapeutic target, wherein the first therapeutic target and the second therapeutic target are different.

### CROSS-REFERENCE TO RELATED APPLICATION

The present application claims priority to U.S. Provisional Application No. 63/310,086, filed Feb. 14, 2022, the entirety of which is incorporated herein by reference.

### SEQUENCE LISTING

The instant application contains a Sequence Listing which has been submitted in XML format and is hereby incorporated by reference in its entirety. Said XML copy, created on Apr. 25, 2023, is named 2017428-0450_SL.xml and is 160,394 bytes in size.

### BACKGROUND

Immunotherapies represent a promising approach to the treatment of various diseases and disorders, including cancer. CAR-T cells, for example, have been used to treat cancers, including B cell malignancies, in humans.

### SUMMARY

Immunotherapies represent a promising approach to the treatment of various diseases and disorders, including cancer. However, the use of an immunotherapy can lead to antigen evasion (also referred to as antigen escape) or antigenic drift. Antigen evasion or antigenic drift arises when a cell targeted by an immunotherapy loses or downregulates an antigen to which the immunotherapy is directed, leading to reduced efficacy of the immunotherapy.

The present disclosure provides the recognition that immunotherapies, such as CAR-T cells, can still provide beneficial treatments, even when a patient is at risk of or is experiencing antigen evasion or antigenic drift. For example, the present disclosure describes that a patient who is at risk of or has undergone antigen evasion or antigenic drift can be administered a therapeutic agent (e.g., comprising one or more populations of engineered cells (e.g., one or more populations of engineered CAR-T cells) that are directed to an antigen that is different than an antigen to which prior-administered immunotherapies directed or to an antigen is that is less susceptible to antigen evasion or antigenic drift. In an exemplary scenario, a patient has previously been administered one or more targeted therapies, wherein the one or more targeted therapies comprised a therapy (e.g., CAR-T cells) directed to CD19. In such a scenario, the present disclosure provides the recognition that the patient can be treated with a therapeutic agent (e.g., engineered cells, e.g., engingeered CAR-T cells) that are directed to CD22. The present disclosure further provides that the patient can be treated with a therapeutic agent (e.g., engineered cells, e.g., engingeered CAR-T cells) that are directed to CD22 and CD19. A therapeutic agent (e.g., engineered cells, e.g., engingeered CAR-T cells) directed to CD22 and CD19 can comprise a population of engineered cells (e.g., engingeered CAR-T cells) that are directed to CD22 and CD19 (e.g., comprise a CAR directed to CD22 and a CAR directed to CD19). A therapeutic agent (e.g., engineered cells, e.g., engingeered CAR-T cells) directed to CD22 and CD19 can also comprise a first population of engineered cells (e.g., engingeered CAR-T cells) that are directed to CD22 (e.g., comprise a CAR directed to CD22) and a second population of engineered cells (e.g., engingeered CAR-T cells) that are directed to CD19 (e.g., comprise a CAR directed to CD19). As another option, a therapeutic agent (e.g., engineered cells, e.g., engingeered CAR-T cells) directed to CD22 and CD19 can comprise a first population of engineered cells (e.g., engingeered CAR-T cells) that are directed to CD22 (e.g., comprise a CAR directed to CD22), a second population of engineered cells (e.g., engingeered CAR-T cells) that are directed to CD19 (e.g., comprise a CAR directed to CD19), and a third population of engineered cells (e.g., engingeered CAR-T cells) that are directed to CD22 and CD19 (e.g., comprise a CAR directed to CD22 and a CAR directed to CD19).

The present disclosure also provides the recognition that off-the-shelf CAR-T cells and other therapeutic cells can offer advantages over autologous cell-based strategies, including ease of manufacturing, quality control and avoidance of malignant contamination and T cell dysfunction. However, the vigorous host-versus-graft immune response against histoincompatible T cells prevents expansion and persistence of allogeneic CAR-T cells and mitigates the efficacy of this approach.

There is substantial evidence in both animal models and human patients that hypoimmunogenic cell transplantation is a scientifically feasible and clinically promising approach to the treatment of numerous disorders, conditions, and diseases.

There remains a need for novel approaches, compositions and methods for producing cell-based therapies that avoid detection by the recipient's immune system.

Provided herein are methods of treating a disease or disorder in a patient. In some embodiments, a disease or disorder is associated with antigen evasion. In some embodiments, a patient has previously been administered one or more targeted therapies directed to a second therapeutic target. In some embodiments, a method comprises administering a population of engineered CAR-T cells to a patient. In some embodiments, a population of engineered CAR-T cells comprises one or more chimeric antigen receptors (CARs). In some embodiments, at least one CAR is directed to the first therapeutic target. In some embodiments, a first therapeutic target and a second therapeutic target are different.

Provided herein are methods of treating a disease or disorder in a patient. In some embodiments, a patient is at risk of antigen evasion. In some embodiments, a patient has previously been administered one or more targeted therapies directed to a second therapeutic target. In some embodiments, a method comprises administering a population of engineered CAR-T cells to a patient. In some embodiments, a population of engineered CAR-T cells comprises one or more chimeric antigen receptors (CARs). In some embodiments, at least one CAR is directed to the first therapeutic target. In some embodiments, a first therapeutic target and a second therapeutic target are different.

In some embodiments, methods of treating a disease or disorder in a patient are provided where the patient has previously been administered one or more targeted therapies directed to a second therapeutic target. In some embodiments, a method comprises administering a therapeutic agent to the patient. In some embodiments, a therapeutic agent comprises a first population of engineered CAR-T cells and a second population of engineered CAR-T cells. In some embodiments, a first population of engineered CAR-T cells comprises one or more chimeric antigen receptors (CARs). In some embodiments, at least one CAR of the first population of engineered CAR-T cells (i) is directed to the first therapeutic target and (ii) comprises a first antigen binding domain. In some embodiments, a second population of engineered CAR-T cells comprises one or more CARs. In some embodiments, at least one CAR of the second population of engineered CAR-T cell (i) is directed to the second therapeutic target and (ii) comprises a second antigen binding domain. In some embodiments, a first therapeutic target and a second therapeutic target are different.

In some embodiments of methods provided herein, a therapeutic agent further comprises a third population of engineered CAR-T cells. In some embodiments, a third population of engineered CAR-T cells comprises two or more CARs. In some embodiments, at least one CAR of the third population of engineered CAR-T cell (i) is directed to the first therapeutic target and (ii) comprises the first antigen binding domain. In some embodiments, at least one CAR of the third population of engineered CAR-T cell (i) is directed to the second therapeutic target, and (ii) comprises the second antigen binding domain.

In some embodiments, a patient has not previously received a therapy directed to the first therapeutic target. In some embodiments, a patient is at risk of antigen evasion.

In some embodiments, a disease or disorder is characterized by antigen evasion. In some embodiments, a disease or disorder is cancer. In some embodiments, a cancer is a lymphoma. In some embodiments, a lymphoma is a B cell lymphoma. In some embodiments, a cancer is a B cell malignancy.

In some embodiments, a first therapeutic target is a first antigen. In some embodiments, a first antigen is an antigen associated with the disease or the disorder. In some embodiments, a first antigen is an antigen present on the surface of a B cell. In some embodiments, a B cell is a malignant B cell. In some embodiments, a first antigen is CD22, CD20, CD19, BCMA, GPRC5D, CD38, CD70, CD79b, HER2, IL13Ra2, or MU. In some embodiments, a first antigen is CD22 or CD20. In some embodiments, a first antigen binding domain is capable of binding to CD22 or CD20.

In some embodiments, a second therapeutic target is a second antigen. In some embodiments, a second antigen is an antigen associated with the disease or the disorder. In some embodiments, a second antigen is an antigen present on the surface of a B cell. In some embodiments, a B cell is a malignant B cell. In some embodiments, a second antigen is CD22, CD20, CD19, BCMA, GPRC5D, CD38, CD70, CD79b, HER2, IL13Ra2, or MU. In some embodiments, a second antigen is CD19. In some embodiments, a second antigen binding domain is capable of binding to CD19.

In some embodiments, a first and/or second population of engineered CAR-T cells comprise reduced expression of a functional major histocompatibility complex class I human leukocyte antigen (HLA-1) complex or reduced expression of a functional major histocompatibility complex class II human leukocyte antigen (HLA-II) complex relative to an unaltered or unmodified wild-type or control cell.

In some embodiments, a first and/or second population of engineered CAR-T cells comprise one or more genetic modifications that reduce expression of one or more HLA-I molecules or one or more HLA-I associated molecules relative to an unaltered or unmodified wild-type or control cell. In some embodiments, a first and/or second population of engineered CAR-T cells do not express one or more HLA-I molecules or one or more HLA-I associated molecules. In some embodiments, a one or more HLA-I associated molecules comprise β-2 microglobulin (B2M).

In some embodiments, a first and/or second population of engineered CAR-T cells comprise one or more genetic modifications that reduce expression of one or more HLA-II molecules or one or more HLA-II associated molecules relative to an unaltered or unmodified wild-type or control cell. In some embodiments, a first and/or second population of engineered CAR-T cells do not express one or more HLA-II molecules or one or more HLA-II associated molecules. In some embodiments, a one or more HLA-II associated molecules comprise CIITA.

In some embodiments, a first and/or second population of engineered CAR-T cells comprise reduced expression of a T cell receptor (TCR) relative to an unaltered or unmodified wild-type or control cell. In some embodiments, a first and/or second population of engineered CAR-T cells do not express TRAC and/or TRBC.

In some embodiments, a first and/or second population of engineered CAR-T cells comprise one or more exogenous polynucleotides that encode one or more tolerogenic factors. In some embodiments, one or more tolerogenic factors comprise A20/TNFAIP3, C1-Inhibitor, CCL21, CCL22, CD16, CD16 Fc receptor, CD24, CD27, CD35, CD39, CD46, CD47, CD52, CD55, CD59, CD64, CD200, CR1, CTLA4-Ig, DUX4, FasL, H2-M3, HLA-C, HLA-E, HLA-E heavy chain, HLA-F, HLA-G, IDO1, IL-10, IL15-RF, IL-35, IL-39, MANF, Mfge8, PD-L1, Serpinb9, CCL21, CCL22, B2M-HLA-E, CI inhibitor, CR1, or a combination thereof. In some embodiments, a first and/or second population of engineered CAR-T cells comprise an exogenous polynucleotide that encode CD47. In some embodiments, a first and/or second population of engineered CAR-T cells comprise CD47, HLA-E, and PD-L1 from one or more exogenous polynucleotides.

In some embodiments, a third population of engineered CAR-T cells comprises reduced expression of a functional major histocompatibility complex class I human leukocyte antigen (HLA-1) complex or reduced expression of a functional major histocompatibility complex class II human leukocyte antigen (HLA-II) complex relative to an unaltered or unmodified wild-type or control cell.

In some embodiments, a third population of engineered CAR-T cells comprises one or more genetic modifications that reduce expression of one or more HLA-I molecules or one or more HLA-I associated molecules relative to an unaltered or unmodified wild-type or control cell. In some embodiments, a third population of engineered CAR-T cells does not express one or more HLA-I molecules or one or more HLA-I associated molecules. In some embodiments, one or more HLA-I associated molecules comprise β-2 microglobulin (B2M).

In some embodiments, a third population of engineered CAR-T cells comprises one or more genetic modifications that reduce expression of one or more HLA-I molecules or one or more HLA-I associated molecules relative to an unaltered or unmodified wild-type or control cell. In some embodiments, a third population of engineered CAR-T cells does not express one or more HLA-II molecules or one or more HLA-II associated molecules. In some embodiments, one or more HLA-II associated molecules comprise CIITA.

In some embodiments, a third population of engineered CAR-T cells comprises reduced expression of a T cell receptor (TCR) relative to an unaltered or unmodified wild-type or control cell. In some embodiments, a third population of engineered CAR-T cells does not express TRAC and/or TRBC.

In some embodiments, a third population of engineered CAR-T cells comprises one or more exogenous polynucleotides that encode one or more tolerogenic factors. In some embodiments, one or more tolerogenic factors comprise A20/TNFAIP3, C1-Inhibitor, CCL21, CCL22, CD16, CD16 Fc receptor, CD24, CD27, CD35, CD39, CD46, CD47, CD52, CD55, CD59, CD64, CD200, CR1, CTLA4-Ig, DUX4, FasL, H2-M3, HLA-C, HLA-E, HLA-E heavy chain, HLA-F, HLA-G, IDO1, IL-10, IL15-RF, IL-35, IL-39, MANF, Mfge8, PD-L1, Serpinb9, CCL21, CCL22, B2M-HLA-E, C1 inhibitor, CR1, or a combination thereof. In some embodiments, a third population of engineered CAR-T cells comprises comprise an exogenous polynucleotide that encode CD47. In some embodiments, a third population of engineered CAR-T cells comprises CD47, HLA-E, and PD-L1 from one or more exogenous polynucleotides.

In some embodiments, provided herein is a method of treating a disease or disorder in a patient who has undergone one or more prior treatments for the disease or disorder, comprising evaluating the patient for the disease or disorder, and administering a population of engineered CAR-T cells to the patient to treat the disease or disorder, wherein the engineered CAR-T cells comprise an exogenous polynucleotide encoding one or more chimeric antigen receptors (CARs), wherein at least one CAR comprises a CD22 antigen binding domain having the CDR sequences of SEQ ID NOs: 47-49 and 51-53, or SEQ ID NOs: 56-58 and 60-62.

Also provided herein is a method of treating a disease or disorder characterized by antigen evasion in a patient who has undergone one or more prior treatments for the disease or disorder prior to antigen evasion, comprising evaluating the patient for the disease or disorder characterized by antigen evasion, and administering a population of engineered CAR-T cells to the patient to treat the disease or disorder characterized by antigen evasion, wherein the engineered CAR-T cells comprise an exogenous polynucleotide encoding one or more CARs, wherein at least one CAR comprises a CD22 antigen binding domain having the CDR sequences of SEQ ID NOs: 47-49 and 51-53, or SEQ ID NOs: 56-58 and 60-62.

In some embodiments, provided herein is a method of treating a cancer characterized by antigen evasion in a patient who has undergone one or more prior treatments for the cancer prior to antigen evasion, comprising evaluating the patient for the disease or disorder characterized by antigen evasion, and administering a population of engineered CAR-T cells to the patient to treat the disease or disorder characterized by antigen evasion, wherein the engineered CAR-T cells comprise an exogenous polynucleotide encoding one or more CARs, wherein at least one CAR comprises a CD22 antigen binding domain having the CDR sequences of SEQ ID NOs: 47-49 and 51-53, or SEQ ID NOs: 56-58 and 60-62.

In some embodiments, provided herein is a method of treating a disease or disorder in a patient who has undergone one or more prior treatments for the disease or disorder, comprising evaluating the patient for the disease or disorder, and administering a population of engineered CAR-T cells to the patient to treat the disease or disorder, wherein the engineered CAR-T cells comprise reduced expression of one or more major histocompatibility complex (MHC) class I and/or class II human leukocyte antigens (HLAs), and reduced expression of a T cell receptor (TCR) relative to an unaltered control cell, and a first exogenous polynucleotide encoding one or more CARs, wherein at least one CAR comprises a CD22 antigen binding domain having the CDR sequences of SEQ ID NOs: 47-49 and 51-53, or SEQ ID NOs: 56-58 and 60-62.

In some embodiments, provided herein is a method of treating a disease or disorder in a patient who has undergone one or more prior treatments for the disease or disorder, comprising evaluating the patient for the disease or disorder, and administering a population of engineered CAR-T cells to the patient to treat the disease or disorder, wherein the engineered CAR-T cells comprise reduced expression of one or more MHC class I and/or class II HLA, and reduced expression of a TCR relative to an unaltered control cell, a first exogenous polynucleotide encoding a tolerogenic factor, and a second exogenous polynucleotide encoding one or more CARs, wherein at least one CAR comprises a CD22 antigen binding domain having the CDR sequences of SEQ ID NOs: 47-49 and 51-53, or SEQ ID NOs: 56-58 and 60-62.

In some embodiments, provided herein is a method of treating a disease or disorder characterized by antigen evasion in a patient who has undergone one or more prior treatments for the disease or disorder prior to antigen evasion, comprising evaluating the patient for the disease or disorder characterized by antigen evasion, and administering a population of engineered CAR-T cells to the patient to treat the disease or disorder, wherein the engineered CAR-T cells comprise reduced expression of one or more MHC class I and/or class II HLA, and reduced expression of a TCR relative to an unaltered control cell, a first exogenous polynucleotide encoding a tolerogenic factor, and a second exogenous polynucleotide encoding one or more CARs, wherein at least one CAR comprises a CD22 antigen binding domain having the CDR sequences of SEQ ID NOs: 47-49 and 51-53, or SEQ ID NOs: 56-58 and 60-62.

In some embodiments, provided herein is a method of treating a cancer characterized by antigen evasion in a patient who has undergone one or more prior treatments for the cancer prior to antigen evasion, comprising evaluating the patient for the disease or disorder characterized by antigen evasion, and administering a population of engineered CAR-T cells to the patient to treat the disease or disorder, wherein the engineered CAR-T cells comprise reduced expression of one or more MHC class I and/or class II HLA, and reduced expression of a TCR relative to an unaltered control cell, a first exogenous polynucleotide encoding a tolerogenic factor, and a second exogenous polynucleotide encoding one or more CARs, wherein at least one CAR comprises a CD22 antigen binding domain having the CDR sequences of SEQ ID NOs: 47-49 and 51-53, or SEQ ID NOs: 56-58 and 60-62.

In some embodiments, the engineered CAR-T cells comprise reduced expression of TCR-alpha (TRAC) and/or TCR-beta (TRBC).

In some embodiments, provided herein is a method of treating a disease or disorder in a patient who has undergone one or more prior treatments for the disease or disorder, comprising evaluating the patient for the disease or disorder, and administering a population of engineered CAR-T cells to the patient to treat the disease or disorder, wherein the engineered CAR-T cells comprise reduced expression of beta-2-microglobulin (B2M) and TRAC, relative to an unaltered control cell, a first exogenous polynucleotide encoding a tolerogenic factor, and a second exogenous polynucleotide encoding one or more CARs, wherein at least one CAR comprises a CD22 antigen binding domain having the CDR sequences of SEQ ID NOs: 47-49 and 51-53, or SEQ ID NOs: 56-58 and 60-62, and wherein the disease or disorder is a cancer.

In some embodiments, the engineered CAR-T cells further comprise reduced expression of MHC class II HLA.

In some embodiments, the engineered CAR-T cells further comprise reduced expression of MHC class 11 transactivator (CIITA).

In some embodiments, the tolerogenic factor is CD47.

In some embodiments, provided herein is a method of treating a disease or disorder in a patient who has undergone one or more prior treatments for the disease or disorder, comprising evaluating the patient for the disease or disorder, and administering a population of engineered CAR-T cells to the patient to treat the disease or disorder, wherein the engineered CAR-T cells comprise reduced expression of B2M, CIITA, and TRAC, relative to an unaltered control cell, a first exogenous polynucleotide encoding CD47, and a second exogenous polynucleotide encoding one or more CARs, wherein at least one CAR comprises a CD22 antigen binding domain having the CDR sequences of SEQ ID NOs: 47-49 and 51-53, or SEQ ID NOs: 56-58 and 60-62, wherein the first exogenous polynucleotide and the second exogenous polynucleotide are inserted by a bicistronic vector, and wherein the disease or disorder is a cancer.

In some embodiments, provided herein is a method of treating a disease or disorder in a patient who has undergone one or more prior treatments for the disease or disorder, comprising evaluating the patient for the disease or disorder, and administering a population of engineered CAR-T cells to the patient to treat the disease or disorder, wherein the engineered CAR-T cells comprise reduced expression of one or more MHC class I and/or class II human leukocyte antigens relative to an unaltered control cell, a first exogenous polynucleotide encoding CD47, and a second exogenous polynucleotide encoding one or more CARs, wherein at least one CAR comprises a CD22 antigen binding domain having the CDR sequences of SEQ ID NOs: 47-49 and 51-53, or SEQ ID NOs: 56-58 and 60-62, and wherein the disease or disorder is a cancer.

In some embodiments, provided herein is a method of treating a disease or disorder in a patient who has undergone one or more prior treatments for the disease or disorder, comprising evaluating the patient for the disease or disorder, and administering a population of engineered CAR-T cells to the patient to treat the disease or disorder, wherein the engineered CAR-T cells comprise reduced expression of B2M relative to an unaltered control cell, a first exogenous polynucleotide encoding a tolerogenic factor, and a second exogenous polynucleotide encoding one or more CARs, wherein at least one CAR comprises a CD22 antigen binding domain having the CDR sequences of SEQ ID NOs: 47-49 and 51-53, or SEQ ID NOs: 56-58 and 60-62, and wherein the disease or disorder is a cancer.

In some embodiments, provided herein is a method of treating a disease or disorder in a patient who has undergone one or more prior treatments for the disease or disorder, comprising evaluating the patient for the disease or disorder, and administering a population of engineered CAR-T cells to the patient to treat the disease or disorder, wherein the engineered CAR-T cells comprise reduced expression of B2M and CIITA relative to an unaltered control cell, a first exogenous polynucleotide encoding a tolerogenic factor, and a second exogenous polynucleotide encoding one or more CARs, wherein at least one CAR comprises a CD22 antigen binding domain having the CDR sequences of SEQ ID NOs: 47-49 and 51-53, or SEQ ID NOs: 56-58 and 60-62, and wherein the disease or disorder is a cancer.

In some embodiments, provided herein is a method of treating a disease or disorder in a patient who has undergone one or more prior treatments for the disease or disorder, comprising evaluating the patient for the disease or disorder, and administering a population of engineered CAR-T cells to the patient to treat the disease or disorder, wherein the engineered CAR-T cells comprise reduced expression of B2M relative to an unaltered control cell, a first exogenous polynucleotide encoding CD47, and a second exogenous polynucleotide encoding one or more CARs, wherein at least one CAR comprises a CD22 antigen binding domain having the CDR sequences of SEQ ID NOs: 47-49 and 51-53, or SEQ ID NOs: 56-58 and 60-62, and wherein the disease or disorder is a cancer.

In some embodiments, provided herein is a method of treating a disease or disorder in a patient who has undergone one or more prior treatments for the disease or disorder, comprising evaluating the patient for the disease or disorder, and administering a population of engineered CAR-T cells to the patient to treat the disease or disorder, wherein the engineered CAR-T cells comprise reduced expression of B2M and CIITA relative to an unaltered control cell, a first exogenous polynucleotide encoding CD47, and a second exogenous polynucleotide encoding one or more CARs, wherein at least one CAR comprises a CD22 antigen binding domain having the CDR sequences from SEQ ID NO: 45, 54, 85, 91, 92, or 93, wherein the first exogenous polynucleotide and the second exogenous polynucleotide are inserted at the same locus, and wherein the disease or disorder is a cancer.

In some embodiments, the CAR has a VH sequence at least 80% identical (e.g., at least 80%, at least 85%, at least 90%, at least 95%, at least 96%, at least 97%, at least 98%, at least 99%, or 100% identical) to the VH sequence of SEQ ID NO: 46 or 55.

In some embodiments, the CAR has a VL sequence at least 80% identical (e.g., at least 80%, at least 85%, at least 90%, at least 95%, at least 96%, at least 97%, at least 98%, at least 99%, or 100% identical) to the VL sequence of SEQ ID NO: 50 or 59.

In some embodiments, the CAR has an scFv sequence at least 80% identical (e.g., at least 80%, at least 85%, at least 90%, at least 95%, at least 96%, at least 97%, at least 98%, at least 99%, or 100% identical) to the scFv sequence of SEQ ID NO: 45, 54, 85, 91, 92, or 93.

In some embodiments, the CAR further comprises one or more of the following components: leader sequence, CD8a signal peptide, linker, m971 binder-based scFv, CD8a hinge domain, CD8 transmembrane domain, CD28 transmembrane domain, 4-1BB costimulatory domain, CD28 signaling domain, CD137 signaling domain, CD8 signaling domain, and CD3ζ signaling domain.

In some embodiments, the CD22 CAR comprises a CD8a transmembrane domain or a CD28 transmembrane domain.

In some embodiments, the CD22 CAR comprises a CD137 signaling domain and a CD3ζ signaling domain.

In some embodiments, the CD22 CAR comprises a CD28 signaling domain and a CD3ζ signaling domain.

In some embodiments, the CD22 CAR comprises a CD28 signaling domain, a CD137 signaling domain, and a CD3ζ signaling domain.

In some embodiments, the CD8a signal peptide comprises the sequence of SEQ ID NO: 6.

In some embodiments, the linker is selected from the group consisting of IgG linkers, Whitlow linkers, (G₄S)ₙ linkers, wherein n is 1, 2, 3, 4, or more, and modifications thereof.

In some embodiments, the linker is a (G₄S)ₙ linker, wherein n is 1 or 3.

In some embodiments, the m971 binder-based scFv comprises CDRs comprising the sequences of SEQ ID NOs: 47-49 and 51-53.

In some embodiments, the m971 binder-based scFv comprises the VH and VL domains of SEQ ID NO: 46 and 50.

In some embodiments, the m971 binder-based scFv comprises the sequence of SEQ ID NO: 45, 54, or 85.

In some embodiments, the m971 binder-based scFv comprises a binder that is functionally equivalent to the m971 binder.

In some embodiments, the m971 binder-based scFv is an m971-L7-based scFv, optionally wherein the m971-L7-based ScFv comprises the sequence of SEQ ID NO: 54.

In some embodiments, the CD8a hinge domain comprises the sequence of SEQ ID NO: 9.

In some embodiments, the CD8 transmembrane domain comprises the sequence of SEQ ID NO: 14 or 86.

In some embodiments, the CD28 transmembrane domain comprises the sequence of SEQ ID NO: 15, 87, or 114.

In some embodiments, the 4-1BB costimulatory domain comprises the sequence of SEQ ID NO: 16.

In some embodiments, the CD28 signaling domain comprises the sequence of SEQ ID NO: 17 or 88.

In some embodiments, the CD137 signaling domain comprises the sequence of SEQ ID NO: 90.

In some embodiments, the CD8 signaling domain comprises the sequence of SEQ ID NO: 89.

In some embodiments, the CD3ζ signaling domain comprises the sequence of SEQ ID NO: 18 or 115.

In some embodiments, the CAR comprises the sequence at least 80% identical (e.g., at least 80%, at least 85%, at least 90%, at least 95%, at least 96%, at least 97%, at least 98%, at least 99%, or 100% identical) to the amino acid sequence of SEQ ID NO: 91, 92, or 93.

In some embodiments, the prior treatments are CD19-specific and/or CD20-specific prior treatments.

In some embodiments, the disease or disorder is characterized by antigen evasion, and wherein the patient has undergone one or more prior treatments for the disease or disorder prior to antigen evasion.

In some embodiments, the disease or disorder is cancer characterized by antigen evasion, and wherein the patient has undergone one or more prior treatments for the cancer prior to antigen evasion.

In some embodiments, the patient is diagnosed as having the disease or disorder prior to administering the population of engineered CAR-T cells.

In some embodiments, the prior treatment comprises an antibody-based therapy, an immune-oncology therapy, or a cell-based therapy.

In some embodiments, the prior treatment comprises a cell-based therapy comprising an autologous CAR-T therapy or an allogeneic CAR-T therapy.

In some embodiments, the prior treatment comprises autologous or allogeneic CAR-T cells expressing a CD22-specific CAR that is the same as, or different from, the CAR expressed by the engineered CAR-T cells.

In some embodiments, the prior treatment comprises autologous or allogeneic CAR-T cells expressing a CD22-specific CAR that is functionally equivalent to the CAR expressed by the engineered CAR-T cells.

In some embodiments, the prior treatment comprises autologous or allogeneic CAR-T cells expressing a CAR that is different from the CAR expressed by the engineered CAR-T cells.

In some embodiments, the prior treatment comprises autologous or allogeneic CD19-CAR-T cells.

In some embodiments, the allogeneic CD19-CAR-T cells comprise a CAR comprising the CDR sequences of SEQ ID NOs: 26-28 and 21-23, or a functionally equivalent CAR thereof.

In some embodiments, the allogeneic CD19-CAR-T cells comprise a CAR comprising the scFv sequence of SEQ ID NO: 19, 29, 32, 34, 36, or 117, or a functionally equivalent CAR thereof.

In some embodiments, the allogeneic CD19-CAR-T cells comprise a CAR comprising the sequence of 32, 34, 36, or 117, or a functionally equivalent CAR thereof.

In some embodiments, the prior treatment comprises axicabtagene ciloleucel, lisocabtagene maraleucel, brexucabtagene autoleucel, or tisagenlecleucel, or a functionally equivalent treatment thereof.

In some embodiments, the prior treatment is a failed prior treatment.

In some embodiments, the failed prior treatment is characterized by one or more of: (a) a plateau or increase in one or more symptom of the disease, (b) a plateau or a worsening of the extent or state of the disease, (c) a plateau or a worsening of disease progression, (d) an attenuated response to therapy, and (e) disease recurrence.

In some embodiments, the antigen binding domain of the one or more CARs binds to one or more antigens associated with the disease or the disorder.

In some embodiments, the disease or disorder is cancer.

In some embodiments, the cancer is a lymphoma, such as a B cell lymphoma.

In some embodiments, the patient is treated with an immunodepleting therapy prior to administering the engineered CAR-T cells.

In some embodiments, the immunodepleting therapy administered prior to administering the engineered CAR-T cells is lower than the immunodepleting therapy administered to the patient prior to the prior treatment.

In some embodiments, the immunodepleting therapy comprises fewer doses than the immunodepleting therapy administered to the patient prior to the prior treatment.

In some embodiments, the immunodepleting therapy comprises a reduced amount of immunodepleting agent than the immunodepleting therapy administered to the patient prior to the prior treatment.

In some embodiments, the immunodepleting therapy comprises administration of fludarabine and/or cyclophosphamide.

In some embodiments, the immunodepleting therapy comprises IV infusion of about 1-50 mg/m² of fludarabine for about 1-7 days.

In some embodiments, the immunodepleting therapy comprises IV infusion of about 1, about 5, about 10, about 20, about 30, about 40, or about 50 mg/m² of fludarabine for about 1, about 2, about 3, about 4, about 5, about 6, or about 7 days.

In some embodiments, the immunodepleting therapy comprises IV infusion of about 30 mg/m² of fludarabine for about 5 days.

In some embodiments, the immunodepleting therapy comprises IV infusion of about 30 mg/m² of fludarabine for about 3 days.

In some embodiments, the immunodepleting therapy comprises IV infusion of about 100-1000 mg/m² of cyclophosphamide for about 1-7 days.

In some embodiments, the immunodepleting therapy comprises IV infusion of about 100, about 200, about 300, about 400, about 500, about 600, about 700, about 800, about 900, or about 1000 mg/m² of cyclophosphamide for about 1, about 2, about 3, about 4, about 5, about 6, or about 7 days.

In some embodiments, the immunodepleting therapy comprises IV infusion of about 500 mg/m² or more of cyclophosphamide for about 5 days.

In some embodiments, the immunodepleting therapy further comprises IV infusion of about 3 mg, about 10 mg, or about 30 mg of alemtuzumab for about 1, about 2, about 3, about 4, about 5, about 6, or about 7 days.

In some embodiments, the immunodepleting therapy comprises IV infusion of about 500 mg/m² of cyclophosphamide for about 3 days.

In some embodiments, the administration is selected from the group consisting of intravenous injection, intramuscular injection, intravascular injection, and transplantation.

In some embodiments, at least about 40×10⁴ engineered CAR-T cells are administered to the patient.

In some embodiments, at least about 40×10⁴ engineered CAR-T cells are administered to the patient.

In some embodiments, up to about 8.0×10⁸ engineered CAR-T cells are administered to the patient, optionally wherein up to about 6.0×10⁸ engineered CAR-T cells are administered to the patient, optionally wherein about 1.0×10⁶ to about 2.5×10⁸ engineered CAR-T cells are administered to the patient or wherein about 2.0×10⁶ to about 2.0×10⁸ engineered CAR-T cells are administered to the patient.

In some embodiments, up to about 6.0×10⁸ engineered CAR-T cells are administered to the patient in about 1-3 doses, optionally wherein (a) about 0.6×10⁶ to about 6.0×10⁸ engineered CAR-T cells are administered to the patient in about 1-3 doses, (b) about 0.2×10⁶ to about 5.0×10⁶ engineered CAR-T cells per kg of the patient's body weight are administered to the patient in about 1-3 doses, if the patient has a body weight of 50 kg or less, (c) about 0.1×10⁸ to about 2.5×10⁸ engineered CAR-T cells are administered to the patient in about 1-3 doses, if the patient has a body weight greater than 50 kg, or (d) about 2.0×10⁶ engineered CAR-T cells per kg of the patient's body weight and up to about 2.0×10⁸ engineered CAR-T cells are administered to the patient in about 1-3 doses.

In some embodiments, about 40×10⁶ to about 200×10⁶ engineered CAR-T cells are administered to the patient, optionally wherein (a) about 40×10⁶ to about 60×10⁶ engineered CAR-T cells are administered to the patient, (b) about 60×10⁶ to about 80×10⁶ engineered CAR-T cells are administered to the patient, (c) about 80×10⁶ to about 100×10⁶ engineered CAR-T cells are administered to the patient, (d) about 100×10⁶ to about 120×10⁶ engineered CAR-T cells are administered to the patient, (e) about 120×10⁶ to about 140×10⁶ engineered CAR-T cells are administered to the patient, (f) about 140×10⁶ to about 160×10⁶ engineered CAR-T cells are administered to the patient, (g) about 160×10⁶ to about 180×10⁶ engineered CAR-T cells are administered to the patient, or (h) about 180×10⁶ to about 200×10⁶ engineered CAR-T cells are administered to the patient.

In some embodiments, about 60×10⁶ to about 120×10⁶ engineered CAR-T cells are administered to the patient, optionally wherein (a) about 60×10⁶ to about 80×10⁶ engineered CAR-T cells are administered to the patient, (b) about 80×10⁶ to about 100×10⁶ engineered CAR-T cells are administered to the patient, or (c) about 100×10⁶ to about 120×10⁶ engineered CAR-T cells are administered to the patient.

In some embodiments, about 120×10⁶ to about 200×10⁶ engineered CAR-T cells are administered to the patient, (a) about 120×10⁶ to about 140×10⁶ engineered CAR-T cells are administered to the patient, (b) about 140×10⁶ to about 160×10⁶ engineered CAR-T cells are administered to the patient, (c) about 160×10⁶ to about 180×10⁶ engineered CAR-T cells are administered to the patient, or (d) about 180×10⁶ to about 200×10⁶ engineered CAR-T cells are administered to the patient.

In some embodiments, the prior treatment comprises an autologous or allogeneic cell-based therapy, and wherein fewer or a lower number of engineered CAR-T cells are administered to the patient than were included in the prior therapy.

In some embodiments, the method further comprises administering a second, third, fourth, fifth, or sixth dose of the engineered CAR-T cells to the patient.

In some embodiments, the patient is not treated with an immunodepleting therapy prior to the second, third, fourth, fifth, and/or sixth administration of the engineered CAR-T cells.

In some embodiments, the patient is treated with an immunodepleting therapy prior to the second, third, fourth, fifth, and/or sixth administration of the engineered CAR-T cells.

In some embodiments, the immunodepleting therapy that is administered prior to the second, third, fourth, fifth, and/or sixth administration of the engineered CAR-T cells is independently selected from administration of fludarabine and/or cyclophosphamide, wherein the administration of fludarabine comprises IV infusion of about 1-50 mg/m² of fludarabine for about 1-7 days, and the administration of cyclophosphamide comprises IV infusion of about 100-1000 mg/m² of cyclophosphamide for about 1-7 days.

In some embodiments, the engineered CAR-T cells are propagated from a primary T cell or a progeny thereof, or are derived from a T cell differentiated from an iPSC or a progeny thereof.

In some embodiments, the engineered CAR-T cells are differentiated cells derived from an induced pluripotent stem cell or a progeny thereof.

In some embodiments, the differentiated cells are a T cells or natural killer (NK) cells.

In some embodiments, the engineered CAR-T cells are a progeny of primary immune cells.

In some embodiments, the progeny of primary immune cells are T cells or NK cells.

In some embodiments, the wild type cell or the control cell is a starting material.

In some embodiments, the engineered CAR-T cells are CAR+ T cells that comprise any one selected from the group consisting of a bulk population of CAR+ T cells, CD4+ CAR+ T cells, CD8+ CAR+ T cells, and a combination thereof.

In some embodiments, the CD4+ CAR+ T cells and CD8+ CAR+ T cells are administered concomitantly or sequentially.

In some embodiments, the CD4+ CAR+ T cells are administered prior to administration of the CD8+ CAR+ T cells, or wherein the CD8+ CAR+ T cells are administered prior to administration of the CD4+ CAR+ T cells.

In some embodiments, the bulk CAR+ T cells and CD8+ CAR+ T cells are administered concomitantly or sequentially.

In some embodiments, the bulk CAR+ T cells are administered prior to administration of the CD8+ CAR+ T cells, or wherein the CD8+ CAR+ T cells are administered prior to administration of the bulk CAR+ T cells.

In some embodiments, the CD4+ CAR+ T cells and bulk CAR+ T cells are administered concomitantly or sequentially.

In some embodiments, the CD4+ CAR+ T cells are administered prior to administration of the bulk CAR+ T cells, or wherein the bulk CAR+ T cells are administered prior to administration of the CD4+ CAR+ T cells.

In some embodiments, the engineered CAR-T cells comprise reduced expression of B2M and/or CIITA relative to an unaltered control cell.

In some embodiments, the engineered CAR-T cells do not express B2M and/or CIITA.

In some embodiments, the engineered CAR-T cells comprise reduced expression of a TCR.

In some embodiments, the engineered CAR-T cells comprise reduced expression of TRAC and/or TRBC.

In some embodiments, the engineered CAR-T cells do not express TRAC and/or TRBC.

In some embodiments, the engineered CAR-T cells comprise reduced expression of HLA class I antigens and/or HLA class II antigens relative to an unaltered control cell.

In some embodiments, the engineered CAR-T cells do not express HLA class I antigens, HLA class II antigens, and/or do not express TCR-alpha.

In some embodiments, the reduced expression or no expression of HLA class I antigens results from the reduced expression or no expression of B2M, and where in the reduced expression or no expression of HLA class II antigens results from the reduced expression or no expression of CIITA.

In some embodiments, the engineered CAR-T cells are B2M^{indel/indel}, CIITA^{indel/indel} cell, and/or a TRAC^{indel/indel}, and/or TRAC^{indel/indel} cells.

In some embodiments, the engineered CAR-T cells comprise reduced expression of HLA-A, HLA-B, HLA-C, HLA-DP, HLA-DQ, HLA-DR, RHD, ABO, PCDH11Y, and/or NLGN4Y relative to an unaltered control cell.

In some embodiments, the engineered CAR-T cells do not express HLA-A, HLA-B, HLA-C, HLA-DP, HLA-DQ, HLA-DR, RHD, ABO, PCDH11Y, and/or NLGN4Y.

In some embodiments, the reduced expression is by way of gene knock down, optionally wherein the gene knock down is by way of RNA silencing or RNA interference (RNAi), optionally selected from the group consisting of short interfering RNAs (siRNAs), PIWI-interacting RNAs (piRNAs), short hairpin RNAs (shRNAs), and microRNAs (miRNAs).

In some embodiments, the reduced expression is by way of gene knock out, optionally wherein the gene knock out is by way of inducing an insertion or a deletion in the gene using a gene editing system, wherein the gene editing system is optionally selected from the group consisting of zinc finger nucleases (ZFNs), transcription activator-like effector nucleases (TALENs), meganucleases, transposases, clustered regularly interspaced short palindromic repeat (CRISPR)/Cas systems, nickase systems, base editing systems, prime editing systems, and gene writing systems.

In some embodiments, the one or more tolerogenic factors are selected from the group consisting of CD47, CD24, CD27, CD35, CD46, CD55, CD59, CD200, HLA-C, HLA-E, HLA-E heavy chain, HLA-G, PD-L1, IDO1, CTLA4-Ig, C1-Inhibitor (e.g., CR1), IL-10, IL-35, FasL, CCL21, CCL22, Mfge8, and Serpinb9.

In some embodiments, the one or more tolerogenic factors comprise CD47.

In some embodiments, provided herein is a method of treating a disease or disorder in a patient who has undergone one or more prior treatments for the disease or disorder, comprising evaluating the patient for the disease or disorder, and administering a population of engineered CAR-T cells to the patient to treat the disease or disorder, wherein the engineered CAR-T cells comprise reduced expression of B2M and TRAC, relative to an unaltered control cell, a first exogenous polynucleotide encoding HLA-E, and a second exogenous polynucleotide encoding one or more CARs, wherein at least one CAR comprises a CD22 antigen binding domain having the CDR sequences from SEQ ID NO: 45, 54, 85, 91, 92, or 93, and wherein the disease or disorder is a cancer.

In some embodiments, the HLA-E is a single chain trimer.

In some embodiments, the HLA-E is a HLA-E/B2M fusion.

In some embodiments, provided herein is a method of treating a disease or disorder in a patient who has undergone one or more prior treatments for the disease or disorder, comprising evaluating the patient for the disease or disorder, and administering a population of engineered CAR-T cells to the patient to treat the disease or disorder, wherein the engineered CAR-T cells comprise reduced expression of B2M and/or CR-1 and TRAC, relative to an unaltered control cell, a first exogenous polynucleotide encoding CD24, and a second exogenous polynucleotide encoding one or more CARs, wherein at least one CAR comprises a CD22 antigen binding domain having the CDR sequences from SEQ ID NO: 45, 54, 85, 91, 92, or 93, and wherein the disease or disorder is a cancer.

In some embodiments, provided herein is a method of treating a disease or disorder in a patient who has undergone one or more prior treatments for the disease or disorder, comprising evaluating the patient for the disease or disorder, and administering a population of engineered CAR-T cells to the patient to treat the disease or disorder, wherein the engineered CAR-T cells comprise reduced expression of B2M and/or CD52 and TRAC, relative to an unaltered control cell, optionally a first exogenous polynucleotide encoding a tolerogenic factor, and a second exogenous polynucleotide encoding one or more CARs, wherein at least one CAR comprises a CD22 antigen binding domain having the CDR sequences of SEQ ID NOs: 47-49 and 51-53, or SEQ ID NOs: 56-58 and 60-62, and wherein the disease or disorder is a cancer.

In some embodiments, provided herein is a method of treating a disease or disorder in a patient who has undergone one or more prior treatments for the disease or disorder, comprising evaluating the patient for the disease or disorder, and administering a population of engineered CAR-T cells to the patient to treat the disease or disorder, wherein the engineered CAR-T cells comprise reduced expression of B2M and/or CD70 and TRAC, relative to an unaltered control cell, optionally a first exogenous polynucleotide encoding a tolerogenic factor, and a second exogenous polynucleotide encoding one or more CARs, wherein at least one CAR comprises a CD22 antigen binding domain having the CDR sequences of SEQ ID NOs: 47-49 and 51-53, or SEQ ID NOs: 56-58 and 60-62, and wherein the disease or disorder is a cancer.

In some embodiments, provided herein is a method of treating a disease or disorder in a patient who has undergone one or more prior treatments for the disease or disorder, comprising evaluating the patient for the disease or disorder, and administering a population of engineered CAR-T cells to the patient to treat the disease or disorder, wherein the engineered CAR-T cells comprise reduced expression of PD-1 and TRAC, relative to an unaltered control cell, optionally a first exogenous polynucleotide encoding a tolerogenic factor, and a second exogenous polynucleotide encoding one or more CARs, wherein at least one CAR comprises a CD22 antigen binding domain having the CDR sequences of SEQ ID NOs: 47-49 and 51-53, or SEQ ID NOs: 56-58 and 60-62, and wherein the disease or disorder is a cancer.

In some embodiments, the engineered CAR-T cells comprise a third exogenous polynucleotide encoding a CD19-specific CAR.

In some embodiments, the CD19-specific CAR comprises a hinge domain of any one of SEQ ID NOs: 9-13, a transmembrane sequence of any one of SEQ ID NOs: 14, 15, and 114, and/or an intracellular costimulatory and/or signaling domain of any one of SEQ ID NOs: 16-18 and 115.

In some embodiments, the first exogenous polynucleotide, the second exogenous polynucleotide, and/or the third exogenous polynucleotides are carried by a polycistronic vector.

In some embodiments, the CD22-specific CAR, the one or more tolerogenic factors, and/or the additional CD19-specific CAR are carried by a single polycistronic vector.

In some embodiments, the polycistronic vector is a bicistronic vector.

In some embodiments, the first, second, and/or third exogenous polynucleotide, and/or the polycistronic vector is inserted into a first, second, and/or third specific locus of at least one allele of the cell.

In some embodiments, the first, second, and/or third specific loci are selected from the group consisting of a safe harbor locus, a target locus, an RHD locus, a B2M locus, a CIITA locus, a TRAC locus, and a TRB locus.

In some embodiments, the safe harbor locus is selected from the group consisting of a CCR5 locus, a PPP1R12C locus, a CLYBL locus, and a Rosa locus.

In some embodiments, the target locus is selected from the group consisting of a CXCR4 locus, an ALB locus, a SHS231 locus, an F3 (CD142) locus, a MICA locus, a MICB locus, a LRP1 (CD91) locus, a HMGB1 locus, an ABO locus, a FUT1 locus, and a KDMSD locus.

In some embodiments, provided herein is a method of treating a disease or disorder in a patient who has undergone one or more prior treatments for the disease or disorder, comprising evaluating the patient for the disease or disorder, and administering a population of engineered CAR-T cells to the patient to treat the disease or disorder, wherein the engineered CAR-T cells comprise reduced expression of B2M, CIITA, and TRAC, relative to an unaltered control cell, a first exogenous polynucleotide encoding CD47, and a second exogenous polynucleotide encoding a CD22 CAR comprising a sequence having at least 90% sequence homology to the sequence set forth in SEQ ID NO: 91, wherein the first exogenous polynucleotide and the second exogenous polynucleotide are inserted by a bicistronic vector, and wherein the disease or disorder is a cancer.

In some embodiments, provided herein is a method of treating a disease or disorder in a patient who has undergone one or more prior treatments for the disease or disorder, comprising evaluating the patient for the disease or disorder, and administering a population of engineered CAR-T cells to the patient to treat the disease or disorder, wherein the engineered CAR-T cells comprise reduced expression of B2M, CIITA, and TRAC, relative to an unaltered control cell, a first exogenous polynucleotide encoding CD47, and a second exogenous polynucleotide encoding a CD22 CAR comprising the sequence set forth in SEQ ID NO: 91, wherein the first exogenous polynucleotide and the second exogenous polynucleotide are inserted by a bicistronic vector, and wherein the disease or disorder is a cancer.

In some embodiments, provided herein is a method of treating a disease or disorder in a patient who has undergone one or more prior treatments for the disease or disorder, comprising evaluating the patient for the disease or disorder, and administering a population of engineered CAR-T cells to the patient to treat the disease or disorder, wherein the engineered CAR-T cells comprise reduced expression of B2M, CIITA, and TRAC, relative to an unaltered control cell, a first exogenous polynucleotide encoding CD47, a second exogenous polynucleotide encoding a CD22 CAR comprising a sequence having at least 90% sequence homology to the sequence set forth in SEQ ID NO: 91, and a third exogenous polynucleotide encoding a CD19 CAR comprising a sequence having at least 90% sequence homology to the sequence set forth in SEQ ID NO: 117 and wherein the disease or disorder is a cancer.

In some embodiments, provided herein is a method of treating a disease or disorder in a patient who has undergone one or more prior treatments for the disease or disorder, comprising evaluating the patient for the disease or disorder, and administering a population of engineered CAR-T cells to the patient to treat the disease or disorder, wherein the engineered CAR-T cells comprise reduced expression of B2M, CIITA, and TRAC, relative to an unaltered control cell, a first exogenous polynucleotide encoding CD47, a second exogenous polynucleotide encoding a CD22 CAR comprising the sequence set forth in SEQ ID NO: 91, and a third exogenous polynucleotide encoding a CD19 CAR comprising a sequence having at least 90% sequence homology to the sequence set forth in SEQ ID NO: 117 and wherein the disease or disorder is a cancer.

In some embodiments, the first exogenous polynucleotide, the second exogenous polynucleotide, and/or the third exogenous polynucleotides are carried by a polycistronic vector.

In some embodiments, the polycistronic vector is a bicistronic vector.

In some embodiments, the first, second, and/or third exogenous polynucleotide or the polycistronic vector is introduced into the engineered CAR-T cells using CRISPR/Cas gene editing.

In some embodiments, the CRISPR/Cas gene editing is carried out ex vivo from a donor patient.

In some embodiments, the first, second, and/or third exogenous polynucleotide, and/or the polycistronic vector is inserted into at least one allele of the engineered CAR-T cell using viral transduction.

In some embodiments, the viral transduction includes a lentivirus based viral vector.

In some embodiments, the lentivirus based viral vector is a pseudotyped, self-inactivating lentiviral vector that carries the first, second, and/or third exogenous polynucleotide, and/or the polycistronic vector.

In some embodiments, the lentivirus based viral vector is a pseudotyped, self-inactivating lentiviral vector that carries the first and second exogenous polynucleotides.

In some embodiments, the lentiviral vector comprises the first exogenous polynucleotide followed by the second exogenous polynucleotide.

In some embodiments, the lentiviral vector comprises the second exogenous polynucleotide followed by the first exogenous polynucleotide.

In some embodiments, the lentivirus based viral vector is a self-inactivating lentiviral vector pseudotyped with a vesicular stomatitis VSV-G envelope and carries the first, second, and/or third exogenous polynucleotide, and/or the polycistronic vector.

In some embodiments, the CD22-specific CAR and/or the CD19-specific CAR are inserted using one or more lentiviral vectors, and the CD47 is inserted using another lentiviral vector.

In some embodiments, the CD22-specific CAR and/or the CD19-specific CAR are inserted using one or more lentiviral vectors, and the CD47 is inserted using a locus-specific insertion method, optionally a CRISPR/Cas or a TALEN method.

In some embodiments, the CD22-specific CAR and/or the CD19-specific CAR are inserted using a locus-specific insertion method, optionally a CRISPR/Cas or a TALEN method, and the CD47 is inserted using a lentiviral vector.

In some embodiments, the CD22-specific CAR and/or the CD19-specific CAR and the CD47 are inserted using one or more lentiviral vectors.

In some embodiments, the CD22-specific CAR and/or the CD19-specific CAR and the CD47 are inserted using a locus-specific insertion method, optionally a CRISPR/Cas or a TALEN method.

In some embodiments, the engineered CAR-T cells evade NK cell mediated cytotoxicity upon administration to the patient.

In some embodiments, the engineered CAR-T cells are protected from cell lysis by mature NK cells upon administration to the patient.

In some embodiments, the engineered CAR-T cells evade macrophage-mediated cytotoxicity, optionally wherein the macrophage-mediated cytotoxicity involves phagocytosis and/or reactive oxygen species.

In some embodiments, the engineered CAR-T cells do not induce an immune response to the cell upon administration to the patient.

In some embodiments, the engineered CAR-T cells persist in the patient for at least 4 weeks, at least 2 months, at least 3 months, at least 4 months, at least 5 months, at least 6 months, at least 7 months, at least 8 months, at least 9 months, at least 10 months, at least 11 months, at least 12 months, or longer.

In some embodiments, the prior treatment comprises an autologous or allogeneic cell-based therapy, and wherein the engineered CAR-T cells persist in the patient for longer than the cells of the prior therapy.

In some embodiments, the therapeutic effect of the engineered CAR-T cells lasts for a duration of at least 4 weeks, at least 2 months, at least 3 months, at least 4 months, at least 5 months, at least 6 months, at least 7 months, at least 8 months, at least 9 months, at least 10 months, at least 11 months, at least 12 months, or longer.

In some embodiments, the therapeutic effect of the engineered CAR-T cells lasts for longer than that of the prior therapy.

In some embodiments, provided herein is a use of a population of engineered CAR-T cells for treating a disease or disorder in a patient who has undergone one or more prior treatments for the disease or disorder, wherein the engineered CAR-T cells comprise an exogenous polynucleotide encoding one or more CARs, wherein at least one CAR comprises a CD22 antigen binding domain having the CDR sequences of SEQ ID NOs: 47-49 and 51-53, or SEQ ID NOs: 56-58 and 60-62.

In some embodiments, provided herein is a use of a population of engineered CAR-T cells for treating a disease or disorder characterized by antigen evasion in a patient who has undergone one or more prior treatments for the disease or disorder prior to antigen evasion, wherein the engineered CAR-T cells comprise an exogenous polynucleotide encoding one or more CARs, wherein at least one CAR comprises a CD22 antigen binding domain having the CDR sequences of SEQ ID NOs: 47-49 and 51-53, or SEQ ID NOs: 56-58 and 60-62.

In some embodiments, provided herein is a use of a population of engineered CAR-T cells for treating a cancer characterized by antigen evasion in a patient who has undergone one or more prior treatments for the cancer prior to antigen evasion, wherein the engineered CAR-T cells comprise an exogenous polynucleotide encoding one or more CARs, wherein at least one CAR comprises a CD22 antigen binding domain having the CDR sequences of SEQ ID NOs: 47-49 and 51-53, or SEQ ID NOs: 56-58 and 60-62.

In some embodiments, provided herein is a use of a population of engineered CAR-T cells for treating a disease or disorder in a patient who has undergone one or more prior treatments for the disease or disorder, wherein the engineered CAR-T cells comprise reduced expression of one or more MHC class I and/or class II HLAs, and reduced expression of a TCR relative to an unaltered control cell, and a first exogenous polynucleotide encoding one or more CARs, wherein at least one CAR comprises a CD22 antigen binding domain having the CDR sequences of SEQ ID NOs: 47-49 and 51-53, or SEQ ID NOs: 56-58 and 60-62.

In some embodiments, provided herein is a use of a population of engineered CAR-T cells for treating a disease or disorder in a patient who has undergone one or more prior treatments for the disease or disorder, wherein the engineered CAR-T cells comprise reduced expression of one or more MHC class I and/or class II HLA, and reduced expression of a TCR relative to an unaltered control cell, a first exogenous polynucleotide encoding a tolerogenic factor, and a second exogenous polynucleotide encoding one or more CARs, wherein at least one CAR comprises a CD22 antigen binding domain having the CDR sequences of SEQ ID NOs: 47-49 and 51-53, or SEQ ID NOs: 56-58 and 60-62.

In some embodiments, provided herein is a use of a population of engineered CAR-T cells for treating a disease or disorder characterized by antigen evasion in a patient who has undergone one or more prior treatments for the disease or disorder prior to antigen evasion, wherein the engineered CAR-T cells comprise reduced expression of one or more MHC class I and/or class II HLA, and reduced expression of a TCR relative to an unaltered control cell, a first exogenous polynucleotide encoding a tolerogenic factor, and a second exogenous polynucleotide encoding one or more CARs, wherein at least one CAR comprises a CD22 antigen binding domain having the CDR sequences of SEQ ID NOs: 47-49 and 51-53, or SEQ ID NOs: 56-58 and 60-62.

In some embodiments, provided herein is a use of a population of engineered CAR-T cells for treating a cancer characterized by antigen evasion in a patient who has undergone one or more prior treatments for the cancer prior to antigen evasion, wherein the engineered CAR-T cells comprise reduced expression of one or more MHC class I and/or class II HLA, and reduced expression of a TCR relative to an unaltered control cell, a first exogenous polynucleotide encoding a tolerogenic factor, and a second exogenous polynucleotide encoding one or more CARs, wherein at least one CAR comprises a CD22 antigen binding domain having the CDR sequences of SEQ ID NOs: 47-49 and 51-53, or SEQ ID NOs: 56-58 and 60-62.

In some embodiments, the engineered CAR-T cells comprise reduced expression of TRAC and/or TRBC.

In some embodiments, provided herein is a use of a population of engineered CAR-T cells for treating a disease or disorder in a patient who has undergone one or more prior treatments for the disease or disorder, wherein the engineered CAR-T cells comprise reduced expression of B2M and TRAC, relative to an unaltered control cell, a first exogenous polynucleotide encoding a tolerogenic factor, and a second exogenous polynucleotide encoding one or more CARs, wherein at least one CAR comprises a CD22 antigen binding domain having the CDR sequences of SEQ ID NOs: 47-49 and 51-53, or SEQ ID NOs: 56-58 and 60-62, and wherein the disease or disorder is a cancer.

In some embodiments, the engineered CAR-T cells further comprise reduced expression of MHC class II HLA.

In some embodiments, the engineered CAR-T cells further comprise reduced expression of CI ITA.

In some embodiments, the tolerogenic factor is CD47.

In some embodiments, provided herein is a use of a population of engineered CAR-T cells for treating a disease or disorder in a patient who has undergone one or more prior treatments for the disease or disorder, wherein the engineered CAR-T cells comprise reduced expression of B2M, CIITA, and TRAC, relative to an unaltered control cell, a first exogenous polynucleotide encoding CD47, and a second exogenous polynucleotide encoding one or more CARs, wherein at least one CAR comprises a CD22 antigen binding domain having the CDR sequences of SEQ ID NOs: 47-49 and 51-53, or SEQ ID NOs: 56-58 and 60-62, wherein the first exogenous polynucleotide and the second exogenous polynucleotide are inserted by a bicistronic vector, and wherein the disease or disorder is a cancer.

In some embodiments, provided herein is a use of a population of engineered CAR-T cells for treating a disease or disorder in a patient who has undergone one or more prior treatments for the disease or disorder, wherein the engineered CAR-T cells comprise reduced expression of one or more MHC class I and/or class II human leukocyte antigens relative to an unaltered control cell, a first exogenous polynucleotide encoding CD47, and a second exogenous polynucleotide encoding one or more CARs, wherein at least one CAR comprises a CD22 antigen binding domain having the CDR sequences of SEQ ID NOs: 47-49 and 51-53, or SEQ ID NOs: 56-58 and 60-62, and wherein the disease or disorder is a cancer.

In some embodiments, provided herein is a use of a population of engineered CAR-T cells for treating a disease or disorder in a patient who has undergone one or more prior treatments for the disease or disorder, wherein the engineered CAR-T cells comprise reduced expression of B2M relative to an unaltered control cell, a first exogenous polynucleotide encoding a tolerogenic factor, and a second exogenous polynucleotide encoding one or more CARs, wherein at least one CAR comprises a CD22 antigen binding domain having the CDR sequences of SEQ ID NOs: 47-49 and 51-53, or SEQ ID NOs: 56-58 and 60-62, and wherein the disease or disorder is a cancer.

In some embodiments, provided herein is a use of a population of engineered CAR-T cells for treating a disease or disorder in a patient who has undergone one or more prior treatments for the disease or disorder, wherein the engineered CAR-T cells comprise reduced expression of B2M and CIITA relative to an unaltered control cell, a first exogenous polynucleotide encoding a tolerogenic factor, and a second exogenous polynucleotide encoding one or more CARs, wherein at least one CAR comprises a CD22 antigen binding domain having the CDR sequences of SEQ ID NOs: 47-49 and 51-53, or SEQ ID NOs: 56-58 and 60-62, and wherein the disease or disorder is a cancer.

In some embodiments, provided herein is a use of a population of engineered CAR-T cells for treating a disease or disorder in a patient who has undergone one or more prior treatments for the disease or disorder, wherein the engineered CAR-T cells comprise reduced expression of B2M relative to an unaltered control cell, a first exogenous polynucleotide encoding CD47, and a second exogenous polynucleotide encoding one or more CARs, wherein at least one CAR comprises a CD22 antigen binding domain having the CDR sequences of SEQ ID NOs: 47-49 and 51-53, or SEQ ID NOs: 56-58 and 60-62, and wherein the disease or disorder is a cancer.

In some embodiments, provided herein is a use of a population of engineered CAR-T cells for treating a disease or disorder in a patient who has undergone one or more prior treatments for the disease or disorder, wherein the engineered CAR-T cells comprise reduced expression of B2M and CIITA relative to an unaltered control cell, a first exogenous polynucleotide encoding CD47, and a second exogenous polynucleotide encoding one or more CARs, wherein at least one CAR comprises a CD22 antigen binding domain having the CDR sequences of SEQ ID NOs: 47-49 and 51-53, or SEQ ID NOs: 56-58 and 60-62, wherein the first exogenous polynucleotide and the second exogenous polynucleotide are inserted at the same locus, and wherein the disease or disorder is a cancer.

In some embodiments, the CAR has a VH sequence at least 80% identical (e.g., at least 80%, at least 85%, at least 90%, at least 95%, at least 96%, at least 97%, at least 98%, at least 99%, or 100% identical) to the VH sequence of SEQ ID NO: 46 or 55.

In some embodiments, the CAR has a VL sequence at least 80% identical (e.g., at least 80%, at least 85%, at least 90%, at least 95%, at least 96%, at least 97%, at least 98%, at least 99%, or 100% identical) to the VL sequence of SEQ ID NO: 50 or 59.

In some embodiments, the CAR has an scFv sequence at least 80% identical (e.g., at least 80%, at least 85%, at least 90%, at least 95%, at least 96%, at least 97%, at least 98%, at least 99%, or 100% identical) to the scFv sequence of SEQ ID NO: 45, 54, 85, 91, 92, or 93.

In some embodiments, the CAR further comprises one or more of the following components: leader sequence, CD8a signal peptide, linker, m971 binder-based scFv, CD8a hinge domain, CD8 transmembrane domain, CD28 transmembrane domain, 4-1BB costimulatory domain, CD28 signaling domain, CD137 signaling domain, CD8 signaling domain, and CD3ζ signaling domain.

In some embodiments, the CD22 CAR comprises a CD8a transmembrane domain or a CD28 transmembrane domain.

In some embodiments, the CD22 CAR comprises a CD137 signaling domain and a CD3ζ signaling domain.

In some embodiments, the CD22 CAR comprises a CD28 signaling domain and a CD3ζ signaling domain.

In some embodiments, the CD22 CAR comprises a CD28 signaling domain, a CD137 signaling domain, and a CD3ζ signaling domain.

In some embodiments, the CD8a signal peptide comprises the sequence of SEQ ID NO: 6.

In some embodiments, the linker is selected from the group consisting of IgG linkers, Whitlow linkers, (G₄S)ₙ linkers, wherein n is 1, 2, 3, 4, or more, and modifications thereof.

In some embodiments, the linker is a (G₄S)ₙ linker, wherein n is 1 or 3.

In some embodiments, the m971 binder-based scFv comprises CDRs comprising the sequences of SEQ ID NOs: 47-49 and 51-53.

In some embodiments, the m971 binder-based scFv comprises the VH and VL domains of SEQ ID NO: 45 and 54.

In some embodiments, the m971 binder-based scFv comprises the sequence of SEQ ID NO: 45, 54, or 85.

In some embodiments, the m971 binder-based scFv comprises a binder that is functionally equivalent to the m971 binder.

In some embodiments, the m971 binder-based scFv is an m971-L7-based scFv, optionally wherein the m971-L7-based ScFv comprises the sequence of SEQ ID NO: 54.

In some embodiments, the CD8a hinge domain comprises the sequence of SEQ ID NO: 9.

In some embodiments, the CD8 transmembrane domain comprises the sequence of SEQ ID NO: 14 or 86.

In some embodiments, the CD28 transmembrane domain comprises the sequence of SEQ ID NO: 15, 87, or 114.

In some embodiments, the 4-1BB costimulatory domain comprises the sequence of SEQ ID NO: 16.

In some embodiments, the CD28 signaling domain comprises the sequence of SEQ ID NO: 17 or 88.

In some embodiments, the CD137 signaling domain comprises the sequence of SEQ ID NO: 90.

In some embodiments, the CD8 signaling domain comprises the sequence of SEQ ID NO: 89.

In some embodiments, the CD3ζ signaling domain comprises the sequence of SEQ ID NO: 18 or 115.

In some embodiments, the CAR comprises the sequence at least 80% identical (e.g., at least 80%, at least 85%, at least 90%, at least 95%, at least 96%, at least 97%, at least 98%, at least 99%, or 100% identical) to the amino acid sequence of SEQ ID NO: 91, 92, or 93.

In some embodiments, the prior treatments are CD19-specific and/or CD20-specific prior treatments.

In some embodiments, the disease or disorder is characterized by antigen evasion, and wherein the patient has undergone one or more prior treatments for the disease or disorder prior to antigen evasion.

In some embodiments, the disease or disorder is cancer characterized by antigen evasion, and wherein the patient has undergone one or more prior treatments for the cancer prior to antigen evasion.

In some embodiments, the patient is diagnosed as having the disease or disorder prior to administering the population of engineered CAR-T cells.

In some embodiments, the prior treatment comprises an antibody-based therapy, an immune-oncology therapy, or a cell-based therapy.

In some embodiments, the prior treatment comprises a cell-based therapy comprising an autologous CAR-T therapy or an allogeneic CAR-T therapy.

In some embodiments, the prior treatment comprises autologous or allogeneic CAR-T cells expressing a CD22-specific CAR that is the same as, or different from, the CAR expressed by the engineered CAR-T cells.

In some embodiments, the prior treatment comprises autologous or allogeneic CAR-T cells expressing a CD22-specific CAR that is functionally equivalent to the CAR expressed by the engineered CAR-T cells.

In some embodiments, the prior treatment comprises autologous or allogeneic CAR-T cells expressing a CAR that is different from the CAR expressed by the engineered CAR-T cells.

In some embodiments, the prior treatment comprises autologous or allogeneic CD19-CAR-T cells.

In some embodiments, the allogeneic CD19-CAR-T cells comprise a CAR comprising the CDR sequences of SEQ ID NOs: 26-28 and 21-23, or a functionally equivalent CAR thereof.

In some embodiments, the allogeneic CD19-CAR-T cells comprise a CAR comprising the scFv sequence of SEQ ID NOd: 19 or 29, or a functionally equivalent CAR thereof.

In some embodiments, the allogeneic CD19-CAR-T cells comprise a CAR comprising the sequence of 32, 34, 36, or 117, or a functionally equivalent CAR thereof.

In some embodiments, the prior treatment comprises axicabtagene ciloleucel, lisocabtagene maraleucel, brexucabtagene autoleucel, or tisagenlecleucel, or a functionally equivalent treatment thereof.

In some embodiments, the prior treatment is a failed prior treatment.

In some embodiments, the failed prior treatment is characterized by one or more of: (a) a plateau or increase in one or more symptom of the disease, (b) a plateau or a worsening of the extent or state of the disease, (c) a plateau or a worsening of disease progression, (d) an attenuated response to therapy, and (e) disease recurrence.

In some embodiments, the antigen binding domain of the one or more CARs binds to one or more antigens associated with the disease or the disorder.

In some embodiments, the disease or disorder is cancer.

In some embodiments, the cancer is a lymphoma, such as a B cell lymphoma.

In some embodiments, the patient is treated with an immunodepleting therapy prior to administering the engineered CAR-T cells.

In some embodiments, the immunodepleting therapy administered prior to administering the engineered CAR-T cells is lower than the immunodepleting therapy administered to the patient prior to the prior treatment.

In some embodiments, the immunodepleting therapy comprises fewer doses than the immunodepleting therapy administered to the patient prior to the prior treatment.

In some embodiments, the immunodepleting therapy comprises a reduced amount of immunodepleting agent than the immunodepleting therapy administered to the patient prior to the prior treatment.

In some embodiments, the immunodepleting therapy comprises administration of fludarabine and/or cyclophosphamide.

In some embodiments, the immunodepleting therapy comprises IV infusion of about 1-50 mg/m² of fludarabine for about 1-7 days.

In some embodiments, the immunodepleting therapy comprises IV infusion of about 1, about 5, about 10, about 20, about 30, about 40, or about 50 mg/m² of fludarabine for about 1, about 2, about 3, about 4, about 5, about 6, or about 7 days.

In some embodiments, the immunodepleting therapy comprises IV infusion of about 30 mg/m² of fludarabine for about 5 days.

In some embodiments, the immunodepleting therapy comprises IV infusion of about 30 mg/m² of fludarabine for about 3 days.

In some embodiments, the immunodepleting therapy comprises IV infusion of about 100-1000 mg/m² of cyclophosphamide for about 1-7 days.

In some embodiments, the immunodepleting therapy comprises IV infusion of about 100, about 200, about 300, about 400, about 500, about 600, about 700, about 800, about 900, or about 1000 mg/m² of cyclophosphamide for about 1, about 2, about 3, about 4, about 5, about 6, or about 7 days.

In some embodiments, the immunodepleting therapy comprises IV infusion of about 500 mg/m² or more of cyclophosphamide for about 5 days.

In some embodiments, the immunodepleting therapy further comprises IV infusion of about 3 mg, about 10 mg, or about 30 mg of alemtuzumab for about 1, about 2, about 3, about 4, about 5, about 6, or about 7 days.

In some embodiments, the immunodepleting therapy comprises IV infusion of about 500 mg/m² of cyclophosphamide for about 3 days.

In some embodiments, the administration is selected from the group consisting of intravenous injection, intramuscular injection, intravascular injection, and transplantation.

In some embodiments, at least about 40×10⁴ engineered CAR-T cells are administered to the patient.

In some embodiments, at least about 40×10⁴ engineered CAR-T cells are administered to the patient.

In some embodiments, up to about 8.0×10⁸ engineered CAR-T cells are administered to the patient, optionally wherein up to about 6.0×10⁸ engineered CAR-T cells are administered to the patient, optionally wherein about 1.0×10⁶ to about 2.5×10⁸ engineered CAR-T cells are administered to the patient or wherein about 2.0×10⁶ to about 2.0×10⁸ engineered CAR-T cells are administered to the patient.

In some embodiments, up to about 6.0×10⁸ engineered CAR-T cells are administered to the patient in about 1-3 doses, optionally wherein (a) about 0.6×10⁶ to about 6.0×10⁸ engineered CAR-T cells are administered to the patient in about 1-3 doses, (b) about 0.2×10⁶ to about 5.0×10⁶ engineered CAR-T cells per kg of the patient's body weight are administered to the patient in about 1-3 doses, if the patient has a body weight of 50 kg or less, (c) about 0.1×10⁸ to about 2.5×10⁸ engineered CAR-T cells are administered to the patient in about 1-3 doses, if the patient has a body weight greater than 50 kg, or (d) about 2.0×10⁶ engineered CAR-T cells per kg of the patient's body weight and up to about 2.0×10⁸ engineered CAR-T cells are administered to the patient in about 1-3 doses.

In some embodiments, about 40×10⁶ to about 200×10⁶ engineered CAR-T cells are administered to the patient, optionally wherein (a) about 40×10⁶ to about 60×10⁶ engineered CAR-T cells are administered to the patient, (b) about 60×10⁶ to about 80×10⁶ engineered CAR-T cells are administered to the patient, (c) about 80×10⁶ to about 100×10⁶ engineered CAR-T cells are administered to the patient, (d) about 100×10⁶ to about 120×10⁶ engineered CAR-T cells are administered to the patient, (e) about 120×10⁶ to about 140×10⁶ engineered CAR-T cells are administered to the patient, (f) about 140×10⁶ to about 160×10⁶ engineered CAR-T cells are administered to the patient, (g) about 160×10⁶ to about 180×10⁶ engineered CAR-T cells are administered to the patient, or (h) about 180×10⁶ to about 200×10⁶ engineered CAR-T cells are administered to the patient.

In some embodiments, about 60×10⁶ to about 120×10⁶ engineered CAR-T cells are administered to the patient, optionally wherein (a) about 60×10⁶ to about 80×10⁶ engineered CAR-T cells are administered to the patient, (b) about 80×10⁶ to about 100×10⁶ engineered CAR-T cells are administered to the patient, or (c) about 100×10⁶ to about 120×10⁶ engineered CAR-T cells are administered to the patient.

In some embodiments, about 120×10⁶ to about 200×10⁶ engineered CAR-T cells are administered to the patient, (a) about 120×10⁶ to about 140×10⁶ engineered CAR-T cells are administered to the patient, (b) about 140×10⁶ to about 160×10⁶ engineered CAR-T cells are administered to the patient, (c) about 160×10⁶ to about 180×10⁶ engineered CAR-T cells are administered to the patient, or (d) about 180×10⁶ to about 200×10⁶ engineered CAR-T cells are administered to the patient.

In some embodiments, the prior treatment comprises an autologous or allogeneic cell-based therapy, and wherein fewer or a lower number of engineered CAR-T cells are administered to the patient than were included in the prior therapy.

In some embodiments, the use further comprises administering a second, third, fourth, fifth, or sixth dose of the engineered CAR-T cells to the patient.

In some embodiments, the patient is not treated with an immunodepleting therapy prior to the second, third, fourth, fifth, and/or sixth administration of the engineered CAR-T cells.

In some embodiments, the patient is treated with an immunodepleting therapy prior to the second, third, fourth, fifth, and/or sixth administration of the engineered CAR-T cells.

In some embodiments, the immunodepleting therapy that is administered prior to the second, third, fourth, fifth, and/or sixth administration of the engineered CAR-T cells is independently selected from administration of fludarabine and/or cyclophosphamide, wherein the administration of fludarabine comprises IV infusion of about 1-50 mg/m² of fludarabine for about 1-7 days, and the administration of cyclophosphamide comprises IV infusion of about 100-1000 mg/m² of cyclophosphamide for about 1-7 days.

In some embodiments, the engineered CAR-T cells are propagated from a primary T cell or a progeny thereof, or are derived from a T cell differentiated from an iPSC or a progeny thereof.

In some embodiments, the engineered CAR-T cells are differentiated cells derived from an induced pluripotent stem cell or a progeny thereof.

In some embodiments, the differentiated cells are a T cells or NK cells.

In some embodiments, the engineered CAR-T cells are a progeny of primary immune cells.

In some embodiments, the progeny of primary immune cells are T cells or NK cells.

In some embodiments, the wild type cell or the control cell is a starting material.

In some embodiments, the engineered CAR-T cells are CAR+ T cells that comprise any one selected from the group consisting of a bulk population of CAR+ T cells, CD4+ CAR+ T cells, CD8+ CAR+ T cells, and a combination thereof.

In some embodiments, the CD4+ CAR+ T cells and CD8+ CAR+ T cells are administered concomitantly or sequentially.

In some embodiments, the CD4+ CAR+ T cells are administered prior to administration of the CD8+ CAR+ T cells, or wherein the CD8+ CAR+ T cells are administered prior to administration of the CD4+ CAR+ T cells.

In some embodiments, the bulk CAR+ T cells and CD8+ CAR+ T cells are administered concomitantly or sequentially.

In some embodiments, the bulk CAR+ T cells are administered prior to administration of the CD8+ CAR+ T cells, or wherein the CD8+ CAR+ T cells are administered prior to administration of the bulk CAR+ T cells.

In some embodiments, the CD4+ CAR+ T cells and bulk CAR+ T cells are administered concomitantly or sequentially.

In some embodiments, the CD4+ CAR+ T cells are administered prior to administration of the bulk CAR+ T cells, or wherein the bulk CAR+ T cells are administered prior to administration of the CD4+ CAR+ T cells.

In some embodiments, the engineered CAR-T cells comprise reduced expression of B2M and/or CIITA relative to an unaltered control cell.

In some embodiments, the engineered CAR-T cells do not express B2M and/or CIITA.

In some embodiments, the engineered CAR-T cells comprise reduced expression of a TCR.

In some embodiments, the engineered CAR-T cells comprise reduced expression of TRAC and/or TRBC.

In some embodiments, the engineered CAR-T cells do not express TRAC and/or TRBC.

In some embodiments, the engineered CAR-T cells comprise reduced expression of HLA class I antigens and/or HLA class II antigens relative to an unaltered control cell.

In some embodiments, the engineered CAR-T cells do not express HLA class I antigens, HLA class II antigens, and/or do not express TCR-alpha.

In some embodiments, the reduced expression or no expression of HLA class I antigens results from the reduced expression or no expression of B2M, and where in the reduced expression or no expression of HLA class II antigens results from the reduced expression or no expression of CIITA.

In some embodiments, the engineered CAR-T cells are B2M^{indel/indel}, CIITA^{indel/indel} cell, and/or a TRAC^{indel/indel}, and/or TRAC^{indel/indel} cells.

In some embodiments, the engineered CAR-T cells comprise reduced expression of HLA-A, HLA-B, HLA-C, HLA-DP, HLA-DQ, HLA-DR, RHD, ABO, PCDH11Y, and/or NLGN4Y relative to an unaltered control cell.

In some embodiments, the engineered CAR-T cells do not express HLA-A, HLA-B, HLA-C, HLA-DP, HLA-DQ, HLA-DR, RHD, ABO, PCDH11Y, and/or NLGN4Y.

In some embodiments, the reduced expression is by way of gene knock down, optionally wherein the gene knock down is by way of RNA silencing or RNAi, optionally selected from the group consisting of siRNAs, piRNAs, shRNAs, and miRNAs.

In some embodiments, the reduced expression is by way of gene knock out, optionally wherein the gene knock out is by way of inducing an insertion or a deletion in the gene using a gene editing system, wherein the gene editing system is optionally selected from the group consisting of ZFNs, TALENs, meganucleases, transposases, CRISPR/Cas systems, nickase systems, base editing systems, prime editing systems, and gene writing systems.

In some embodiments, the one or more tolerogenic factors are selected from the group consisting of CD47, CD24, CD27, CD35, CD46, CD55, CD59, CD200, HLA-C, HLA-E, HLA-E heavy chain, HLA-G, PD-L1, IDO1, CTLA4-Ig, C1-Inhibitor (e.g., CR1), IL-10, IL-35, FasL, CCL21, CCL22, Mfge8, and Serpinb9.

In some embodiments, the one or more tolerogenic factors comprise CD47.

In some embodiments, provided herein is a use of a population of engineered CAR-T cells for treating a disease or disorder in a patient who has undergone one or more prior treatments for the disease or disorder, wherein the engineered CAR-T cells comprise reduced expression of B2M and TRAC, relative to an unaltered control cell, a first exogenous polynucleotide encoding HLA-E, and a second exogenous polynucleotide encoding one or more CARs, wherein at least one CAR comprises a CD22 antigen binding domain having the CDR sequences of SEQ ID NOs: 47-49 and 51-53, or SEQ ID NOs: 56-58 and 60-62, and wherein the disease or disorder is a cancer.

In some embodiments, the HLA-E is a single chain trimer.

In some embodiments, the HLA-E is a HLA-E/B2M fusion.

In some embodiments, provided herein is a use of a population of engineered CAR-T cells for treating a disease or disorder in a patient who has undergone one or more prior treatments for the disease or disorder, wherein the engineered CAR-T cells comprise reduced expression of B2M and/or CR-1 and TRAC, relative to an unaltered control cell, a first exogenous polynucleotide encoding CD24, and a second exogenous polynucleotide encoding one or more CARs, wherein at least one CAR comprises a CD22 antigen binding domain having the CDR sequences of SEQ ID NOs: 47-49 and 51-53, or SEQ ID NOs: 56-58 and 60-62, and wherein the disease or disorder is a cancer.

In some embodiments, provided herein is a use of a population of engineered CAR-T cells for treating a disease or disorder in a patient who has undergone one or more prior treatments for the disease or disorder, wherein the engineered CAR-T cells comprise reduced expression of B2M and/or CD52 and TRAC, relative to an unaltered control cell, optionally a first exogenous polynucleotide encoding a tolerogenic factor, and a second exogenous polynucleotide encoding one or more CARs, wherein at least one CAR comprises a CD22 antigen binding domain having the CDR sequences of SEQ ID NOs: 47-49 and 51-53, or SEQ ID NOs: 56-58 and 60-62, and wherein the disease or disorder is a cancer.

In some embodiments, provided herein is a use of a population of engineered CAR-T cells for treating a disease or disorder in a patient who has undergone one or more prior treatments for the disease or disorder, wherein the engineered CAR-T cells comprise reduced expression of B2M and/or CD70 and TRAC, relative to an unaltered control cell, optionally a first exogenous polynucleotide encoding a tolerogenic factor, and a second exogenous polynucleotide encoding one or more CARs, wherein at least one CAR comprises a CD22 antigen binding domain having the CDR sequences of SEQ ID NOs: 47-49 and 51-53, or SEQ ID NOs: 56-58 and 60-62, and wherein the disease or disorder is a cancer.

In some embodiments, provided herein is a use of a population of engineered CAR-T cells for treating a disease or disorder in a patient who has undergone one or more prior treatments for the disease or disorder, wherein the engineered CAR-T cells comprise reduced expression of PD-1 and TRAC, relative to an unaltered control cell, optionally a first exogenous polynucleotide encoding a tolerogenic factor, and a second exogenous polynucleotide encoding one or more CARs, wherein at least one CAR comprises a CD22 antigen binding domain having the CDR sequences of SEQ ID NOs: 47-49 and 51-53, or SEQ ID NOs: 56-58 and 60-62, and wherein the disease or disorder is a cancer.

In some embodiments, the engineered CAR-T cells comprise a third exogenous polynucleotide encoding a CD19-specific CAR.

In some embodiments, the CD19-specific CAR comprises a hinge domain of any one of SEQ ID NOs: 9-13, a transmembrane sequence of any one of SEQ ID NOs: 14, 15, and 114, and/or an intracellular costimulatory and/or signaling domain of any one of SEQ ID NOs: 16-18 and 115.

In some embodiments, the first exogenous polynucleotide, the second exogenous polynucleotide, and/or the third exogenous polynucleotides are carried by a polycistronic vector.

In some embodiments, the CD22-specific CAR, the one or more tolerogenic factors, and/or the additional CD19-specific CAR are carried by a single polycistronic vector.

In some embodiments, the polycistronic vector is a bicistronic vector.

In some embodiments, the first, second, and/or third exogenous polynucleotide, and/or the polycistronic vector is inserted into a first, second, and/or third specific locus of at least one allele of the cell.

In some embodiments, the first, second, and/or third specific loci are selected from the group consisting of a safe harbor locus, a target locus, an RHD locus, a B2M locus, a CIITA locus, a TRAC locus, and a TRB locus.

In some embodiments, the safe harbor locus is selected from the group consisting of a CCR5 locus, a PPP1R12C locus, a CLYBL locus, and a Rosa locus.

In some embodiments, the target locus is selected from the group consisting of a CXCR4 locus, an ALB locus, a SHS231 locus, an F3 (CD142) locus, a MICA locus, a MICB locus, a LRP1 (CD91) locus, a HMGB1 locus, an ABO locus, a FUT1 locus, and a KDMSD locus.

In some embodiments, provided herein is a use of a population of engineered CAR-T cells for treating a disease or disorder in a patient who has undergone one or more prior treatments for the disease or disorder, wherein the engineered CAR-T cells comprise reduced expression of B2M, CIITA, and TRAC, relative to an unaltered control cell, a first exogenous polynucleotide encoding CD47, and a second exogenous polynucleotide encoding a CD22 CAR comprising a sequence having at least 90% sequence homology to the sequence set forth in SEQ ID NO: 91, wherein the first exogenous polynucleotide and the second exogenous polynucleotide are inserted by a bicistronic vector, and wherein the disease or disorder is a cancer.

In some embodiments, provided herein is a use of a population of engineered CAR-T cells for treating a disease or disorder in a patient who has undergone one or more prior treatments for the disease or disorder, wherein the engineered CAR-T cells comprise reduced expression of B2M, CIITA, and TRAC, relative to an unaltered control cell, a first exogenous polynucleotide encoding CD47, and a second exogenous polynucleotide encoding a CD22 CAR comprising the sequence set forth in SEQ ID NO: 91, wherein the first exogenous polynucleotide and the second exogenous polynucleotide are inserted by a bicistronic vector, and wherein the disease or disorder is a cancer.

In some embodiments, provided herein is a use of a population of engineered CAR-T cells for treating a disease or disorder in a patient who has undergone one or more prior treatments for the disease or disorder, wherein the engineered CAR-T cells comprise reduced expression of B2M, CIITA, and TRAC, relative to an unaltered control cell, a first exogenous polynucleotide encoding CD47, a second exogenous polynucleotide encoding a CD22 CAR comprising a sequence having at least 90% sequence homology to the sequence set forth in SEQ ID NO: 91, and a third exogenous polynucleotide encoding a CD19 CAR comprising a sequence having at least 90% sequence homology to the sequence set forth in SEQ ID NO: 117 and wherein the disease or disorder is a cancer.

In some embodiments, provided herein is a use of a population of engineered CAR-T cells for treating a disease or disorder in a patient who has undergone one or more prior treatments for the disease or disorder, wherein the engineered CAR-T cells comprise reduced expression of B2M, CIITA, and TRAC, relative to an unaltered control cell, a first exogenous polynucleotide encoding CD47, a second exogenous polynucleotide encoding a CD22 CAR comprising the sequence set forth in SEQ ID NO: 91, and a third exogenous polynucleotide encoding a CD19 CAR comprising a sequence having at least 90% sequence homology to the sequence set forth in SEQ ID NO: 117 and wherein the disease or disorder is a cancer.

In some embodiments, the first exogenous polynucleotide, the second exogenous polynucleotide, and/or the third exogenous polynucleotides are carried by a polycistronic vector.

In some embodiments, the polycistronic vector is a bicistronic vector.

In some embodiments, the first, second, and/or third exogenous polynucleotide or the polycistronic vector is introduced into the engineered CAR-T cells using CRISPR/Cas gene editing.

In some embodiments, the CRISPR/Cas gene editing is carried out ex vivo from a donor patient.

In some embodiments, the first, second, and/or third exogenous polynucleotide, and/or the polycistronic vector is inserted into at least one allele of the engineered CAR-T cell using viral transduction.

In some embodiments, the viral transduction includes a lentivirus based viral vector.

In some embodiments, the lentivirus based viral vector is a pseudotyped, self-inactivating lentiviral vector that carries the first, second, and/or third exogenous polynucleotide, and/or the polycistronic vector.

In some embodiments, the lentivirus based viral vector is a pseudotyped, self-inactivating lentiviral vector that carries the first and second exogenous polynucleotides.

In some embodiments, the lentiviral vector comprises the first exogenous polynucleotide followed by the second exogenous polynucleotide.

In some embodiments, the lentiviral vector comprises the second exogenous polynucleotide followed by the first exogenous polynucleotide.

In some embodiments, the lentivirus based viral vector is a self-inactivating lentiviral vector pseudotyped with a vesicular stomatitis VSV-G envelope and carries the first, second, and/or third exogenous polynucleotide, and/or the polycistronic vector.

In some embodiments, the CD22-specific CAR and/or the CD19-specific CAR are inserted using one or more lentiviral vectors, and the CD47 is inserted using another lentiviral vector.

In some embodiments, the CD22-specific CAR and/or the CD19-specific CAR are inserted using one or more lentiviral vectors, and the CD47 is inserted using a locus-specific insertion method, optionally a CRISPR/Cas or a TALEN method.

In some embodiments, the CD22-specific CAR and/or the CD19-specific CAR are inserted using a locus-specific insertion method, optionally a CRISPR/Cas or a TALEN method, and the CD47 is inserted using a lentiviral vector.

In some embodiments, the CD22-specific CAR and/or the CD19-specific CAR and the CD47 are inserted using one or more lentiviral vectors.

In some embodiments, the CD22-specific CAR and/or the CD19-specific CAR and the CD47 are inserted using a locus-specific insertion method, optionally a CRISPR/Cas or a TALEN method.

In some embodiments, the engineered CAR-T cells evade NK cell mediated cytotoxicity upon administration to the patient.

In some embodiments, the engineered CAR-T cells are protected from cell lysis by mature NK cells upon administration to the patient.

In some embodiments, the engineered CAR-T cells evade macrophage-mediated cytotoxicity, optionally wherein the macrophage-mediated cytotoxicity involves phagocytosis and/or reactive oxygen species.

In some embodiments, the engineered CAR-T cells do not induce an immune response to the cell upon administration to the patient.

In some embodiments, the engineered CAR-T cells persist in the patient for at least 4 weeks, at least 2 months, at least 3 months, at least 4 months, at least 5 months, at least 6 months, at least 7 months, at least 8 months, at least 9 months, at least 10 months, at least 11 months, at least 12 months, or longer.

In some embodiments, the prior treatment comprises an autologous or allogeneic cell-based therapy, and wherein the engineered CAR-T cells persist in the patient for longer than the cells of the prior therapy.

In some embodiments, the therapeutic effect of the engineered CAR-T cells lasts for a duration of at least 4 weeks, at least 2 months, at least 3 months, at least 4 months, at least 5 months, at least 6 months, at least 7 months, at least 8 months, at least 9 months, at least 10 months, at least 11 months, at least 12 months, or longer.

In some embodiments, the therapeutic effect of the engineered CAR-T cells lasts for longer than that of the prior therapy.

Detailed descriptions of engineered and/or hypoimmunogenic cells, methods of producing thereof, and methods of using thereof are found in U.S. Provisional Application No. 63/065,342 filed on Aug. 13, 2020, WO2016/183041 filed May 9, 2015, WO2018/132783 filed Jan. 14, 2018, WO2020/018615 filed Jul. 17, 2019, WO2020/018620 filed Jul. 17, 2019, WO2020/168317 filed Feb. 16, 2020, the disclosures of which including the examples, sequence listings and figures are incorporated herein by reference in their entireties.

### Definitions

As described in the present disclosure, the following terms will be employed, and are defined as indicated below.

Antigen Evasion orAntigen Escape: As used herein, “antigen evasion,” “antigen escape,” and variations thereof refer to reduced or loss of expression of a target antigen. In some embodiments, a cancer that has undergone antigen evasion is a cancer that was positive for an antigen and exhibits reduced or loss of expression of the antigen following a therapy targeted at that antigen. For example, a cancer that has undergone antigen evasion is a cancer that was CD19-positive and has exhibited reduced or loss of expression of CD19. In some embodiments, a cancer that has undergone antigen evasion is a cancer that was CD19-positive and has changed its antigen profile to instead express CD22, following a CD19-targeted therapy resulting in CD19-targeted therapy failure. In some embodiments, the CD19-targeted therapy is a CD19 CAR-T therapy.

Cancer: The term “cancer” as used herein is defined as a hyperproliferation of cells whose unique trait (e.g., loss of normal controls) results in unregulated growth, lack of differentiation, local tissue invasion, and metastasis. With respect to the inventive methods, the cancer can be any cancer, including any of acute lymphocytic cancer, acute myeloid leukemia, lymphoma, leukemia, B-cell acute lymphoblastic leukemia (B-ALL), B-cell Non-Hodgkin lymphoma (B-NHL), B-cell chronic lymphoblastic leukemia, alveolar rhabdomyosarcoma, bladder cancer, bone cancer, brain cancer, breast cancer, cancer of the anus, anal canal, or anorectum, cancer of the eye, cancer of the intrahepatic bile duct, cancer of the joints, cancer of the neck, gallbladder, or pleura, cancer of the nose, nasal cavity, or middle ear, cancer of the oral cavity, cancer of the vulva, chronic lymphocytic leukemia, chronic myeloid cancer, colon cancer, esophageal cancer, cervical cancer, fibrosarcoma, gastrointestinal carcinoid tumor, Hodgkin lymphoma, hypopharynx cancer, kidney cancer, larynx cancer, leukemia, liquid tumors, liver cancer, lung cancer, lymphoma, malignant mesothelioma, mastocytoma, melanoma, multiple myeloma, nasopharynx cancer, non-Hodgkin lymphoma, ovarian cancer, pancreatic cancer, peritoneum, omentum, and mesentery cancer, pharynx cancer, prostate cancer, rectal cancer, renal cancer, skin cancer, small intestine cancer, soft tissue cancer, solid tumors, stomach cancer, testicular cancer, thyroid cancer, ureter cancer, and/or urinary bladder cancer. In some embodiments, any of the exemplary cancers are also a CD19-negative cancer, a CD22-positive cancer, a CD19-negative/CD22-positive cancer, or a CD19-positive cancer. In certain embodiments, any of the exemplary cancers underwent antigen evasion and no longer express an antigen or have reduced expression of an antigen previously expressed. For example, any of the exemplary cancers can be a CD19-negative and a CD22-positive cancer but were previously CD19-positive and CD22-negative or CD22-positive. As used herein, the term “tumor” refers to an abnormal growth of cells or tissues of the malignant type, unless otherwise specifically indicated and does not include a benign type tissue.

Clinically Effective Amount: As used herein, “clinically effective amount” refers to an amount sufficient to provide a clinical benefit in the treatment and/or management of a disease, disorder, or condition. In some embodiments, a clinically effective amount is an amount that has been shown to produce at least one improved clinical endpoint to the standard of care for the disease, disorder, or condition. In some embodiments, a clinically effective amount is an amount that has been demonstrated, for example in a clinical trial, to be sufficient to provide statistically significant and meaningful effectiveness for treating the disease, disorder, or condition. In some embodiments, the clinically effective amount is also a therapeutically effective amount. In other embodiments, the clinically effective amount is not a therapeutically effective amount.

Complementarity Determining Region: As used herein, the term “CDR” or “complementarity determining region” means a non-contiguous antigen binding site present in the variable domain of each of the heavy and light chain polypeptides. CDRs were identified according to the following rules deduced from Kabat et al. (1991) and Chotia and Lesk (1987), both of which are incorporated by reference herein in their entirety. Exemplary rules for determining CDRs are included below:
LCDR1:
   Start is at approximately residue 24
   Residue before LCDR1 is Cys
   The residue after LCDR1 is Trp; typically, as part of the sequences TRP-TYR-GLN, but may be TRP-LEU-GLN, TRP-PHE-GLN, TRP-TYR-LEU.
   Length 10 to 17 residues
LCDR2:
   Start residue is about 16 residues after the end of the start of LCDR1
   Residues before are generally ILE-TYR, but may be VAL-TYR, ILE-LYS, ILE-PHE
   Length is 7 residues in length
LCDR3:
   Start residue is about 33 residues after the end of LCDR2
   The residue before LCDR3 is Cys
   Residues after LCDR2 include PHE-GLY-XXX-GLY
   Length 7-11 residues
HCDR1:
   Start residue is approximately residue 26 (4 residues after CYS according to Chothia/AbM
   definition) (Kabat definition starts 5 residues later)
   The sequence before HCDR1 is CYS-XXX-XXX-XXX
   The residue after HCDR1 is TRP. Typically TRP-VAL, but may be TRP-ILE, TRP-ALA
   Length is 10-12 residues (AbM definition), Chothia definition excludes the last 4 residues
HCDR2:
   Start residue is 15 residues after HCDR1 (Kabat/AbM definition)
   Residues before HCDR2 are typically LEU-GLU-TRP-ILE-GLY, but many variations are possible
   The residues after HCDR2 are LYS, ARG-LEU, ILE, VAL, PHE, THR, ALA-THR, SER, ILE, ALA
   Length is 16-19 residues as defined by Kabat (AbM definition ends before 7 residues)
HCDR3:
   Start residue is 33 residues after the end of HCDR2 (2 residues after CYS)
   The sequence before HCDR3 is CYS-XXX-XXX (typically CYS-ALA-ARG)
   The residue after HCDR3 is TRP-GLY-XXX-GLY
   3-25 residues in length

Decrease, Reduce, and Reduction: The terms “decrease,” “reduce,” and “reduction,” as well as grammatical variations thereof, are all used herein generally to mean a decrease by a statistically significant amount. However, for avoidance of doubt, decrease,” “reduced,” “reduction,” “decrease” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level. In some embodiments, the cells are engineered to have reduced expression of one or more targets relative to an unaltered or unmodified wild-type cell.

Derived from an iPSC or a Progeny Thereof: In some embodiments, the engineered and hypoimmunogenic cells described are derived from an iPSC or a progeny thereof. As used herein, the term “derived from an iPSC or a progeny thereof” encompasses the initial iPSC that is generated and any subsequent progeny thereof.

Directed to: As used herein, when an entity is “directed to” a target, the entity selectively interacts with the target. The fact that an entity is directed to a target does not mean that the entity does not interact with any other molecules or entities; rather, it means that, regardless of what else the entity interacts with it is able to selectively interact with the target. In some embodiments, an entity that is directed to a target may selectively bind to the target. In some embodiments, an entity that is directed to a target may specifically bind to the target.

Donor or Donor Subject: The term “donor” or “donor subject” refer to an animal, for example, a human from whom cells can be obtained. The “non-human animals” and “non-human mammals” as used interchangeably herein, includes mammals such as rats, mice, rabbits, sheep, cats, dogs, cows, pigs, and non-human primates. The term “donor subject” also encompasses any vertebrate including but not limited to mammals, reptiles, amphibians and fish. However, advantageously, the donor subject is a mammal such as a human, or other mammals such as a domesticated mammal, e.g., dog, cat, horse, and the like, or production mammal, e.g., cow, sheep, pig, and the like. A “donor subject” can also refer to more than one donor, for example one or more humans or non-human animals or non-human mammals.

Endogenous: The term “endogenous” refers to a referenced molecule or polypeptide that is naturally present in the cell. Similarly, the term when used in reference to expression of an encoding nucleic acid refers to expression of an encoding nucleic acid naturally contained within the cell and not exogenously introduced. Similarly, the term when used in reference to a promoter sequence refers to a promoter sequence naturally contained within the cell and not exogenously introduced.

Engineered Cell: The term “engineered cell” as used herein refers to a cell that has been altered in at least some way by human intervention, including, for example, by genetic alterations or modifications such that the engineered cell differs from a wild-type cell.

Exogenous: As used herein, the term “exogenous” in the context of a polynucleotide or polypeptide being expressed is intended to mean that the referenced molecule or the referenced polypeptide is introduced into the cell of interest. The polypeptide can be introduced, for example, by introduction of an encoding nucleic acid into the genetic material of the cells such as by integration into a chromosome or as non-chromosomal genetic material such as a plasmid or expression vector. Therefore, the term as it is used in reference to expression of an encoding nucleic acid refers to introduction of the encoding nucleic acid in an expressible form into the cell. An exogenous polynucleotide can be inserted into at least one allele of the cell using viral transduction, for example, with a vector. In some embodiments, the vector is a pseudotyped, self-inactivating lentiviral vector that carries exogenous polynucleotide. In some embodiments, the vector is a self-inactivating lentiviral vector pseudotyped with a vesicular stomatitis VSV-G envelope, and which carries the exogenous polynucleotide. In some embodiments, the exogenous polynucleotide is inserted into at least one allele of the cell using viral transduction. In some embodiments, exogenous polynucleotide is inserted into at least one allele of the cell using a lentivirus based viral vector. In some embodiments, the exogenous polynucleotide is inserted into a safe harbor or target locus of at least one allele of the cell.

An “exogenous” molecule is a molecule, construct, factor and the like that is not normally present in a cell, but can be introduced into a cell by one or more genetic, biochemical or other methods. “Normal presence in the cell” is determined with respect to the particular developmental stage and environmental conditions of the cell. Thus, for example, a molecule that is present only during embryonic development of neurons is an exogenous molecule with respect to an adult neuron cell. An exogenous molecule can comprise, for example, a functioning version of a malfunctioning endogenous molecule or a malfunctioning version of a normally-functioning endogenous molecule.

An exogenous molecule or factor can be, among other things, a small molecule, such as is generated by a combinatorial chemistry process, or a macromolecule such as a protein, nucleic acid, carbohydrate, lipid, glycoprotein, lipoprotein, polysaccharide, any modified derivative of the above molecules, or any complex comprising one or more of the above molecules. Nucleic acids include DNA and RNA, can be single- or double-stranded; can be linear, branched or circular; and can be of any length. Nucleic acids include those capable of forming duplexes, as well as triplex-forming nucleic acids. See, for example, U.S. Pat. Nos. 5,176,996 and 5,422,251. Proteins include, but are not limited to, DNA-binding proteins, transcription factors, chromatin remodeling factors, methylated DNA binding proteins, polymerases, methylases, demethylases, acetylases, deacetylases, kinases, phosphatases, integrases, recombinases, ligases, topoisomerases, gyrases and helicases.

An exogenous molecule or construct can be the same type of molecule as an endogenous molecule, e.g., an exogenous protein or nucleic acid. In such instances, the exogenous molecule is introduced into the cell at greater concentrations than that of the endogenous molecule in the cell. In some instances, an exogenous nucleic acid can comprise an infecting viral genome, a plasmid or episome introduced into a cell, or a chromosome that is not normally present in the cell. Methods for the introduction of exogenous molecules into cells are known to those of skill in the art and include, but are not limited to, lipid-mediated transfer (i.e., liposomes, including neutral and cationic lipids), electroporation, direct injection, cell fusion, particle bombardment, calcium phosphate co-precipitation, DEAE-dextran-mediated transfer and viral vector-mediated transfer.

Gene: A “gene,” for the purposes of the present disclosure, includes a DNA region encoding a gene product, as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and/or locus control regions.

Gene Expression: “Gene expression” refers to the conversion of the information, contained in a gene, into a gene product. A gene product can be the direct transcriptional product of a gene (e.g., mRNA, tRNA, rRNA, antisense RNA, ribozyme, structural RNA or any other type of RNA) or a protein produced by translation of an mRNA. Gene products also include RNAs which are modified, by processes such as capping, polyadenylation, methylation, and editing, and proteins modified by, for example, methylation, acetylation, phosphorylation, ubiquitination, ADP-ribosylation, myristoylation, and/or glycosylation.

Genetic Modification: The term “genetic modification” and its grammatical equivalents as used herein can refer to one or more alterations of a nucleic acid, e.g., the nucleic acid within an organism's genome. For example, genetic modification can refer to alterations, additions, and/or deletion of genes or portions of genes or other nucleic acid sequences. A genetically modified cell can also refer to a cell with an added, deleted and/or altered gene or portion of a gene. A genetically modified cell can also refer to a cell with an added nucleic acid sequence that is not a gene or gene portion. Genetic modifications include, for example, both transient knock-in or knock-down mechanisms, and mechanisms that result in permanent knock-in, knock-down, or knock-out of target genes or portions of genes or nucleic acid sequences Genetic modifications include, for example, both transient knock-in and mechanisms that result in permanent knock-in of nucleic acids sequences Genetic modifications also include, for example, reduced or increased transcription, reduced or increased mRNA stability, reduced or increased translation, and reduced or increased protein stability.

In additional or alternative aspects, the present disclosure contemplates altering target polynucleotide sequences in any manner which is available to the skilled artisan, e.g., utilizing a nuclease system such as a TAL effector nuclease (TALEN) or zinc finger nuclease (ZFN) system. It should be understood that although examples of methods utilizing CRISPR/Cas (e.g., Cas9 and Cas12a) and TALEN are described in detail herein, the disclosure is not limited to the use of these methods/systems. Other methods of targeting to reduce or ablate expression in target cells known to the skilled artisan can be utilized herein. The methods provided herein can be used to alter a target polynucleotide sequence in a cell. The present disclosure contemplates altering target polynucleotide sequences in a cell for any purpose. In some embodiments, the target polynucleotide sequence in a cell is altered to produce a mutant cell. In some embodiments, an alteration or modification (including, for example, genetic alterations or modifications) described herein results in reduced expression of a target or selected polynucleotide sequence. In some embodiments, an alteration or modification described herein results in reduced expression of a target or selected polypeptide sequence. In some embodiments, an alteration or modification described herein results in increased expression of a target or selected polynucleotide sequence. In some embodiments, an alteration or modification described herein results in increased expression of a target or selected polypeptide sequence.

Grafting, Administering, Introducing, Implanting, and Transplanting: As used herein, the terms “grafting,” “administering,” “introducing,” “implanting” and “transplanting,” as well as grammatical variations thereof, are used interchangeably in the context of the placement of cells (e.g., cells described herein) into a subject, by a method or route which results in localization or at least partial localization of the introduced cells at a desired site or systemic introduction (e.g., into circulation). The cells can be implanted directly to the desired site, or alternatively be administered by any appropriate route which results in delivery to a desired location in the subject where at least a portion of the implanted cells or components of the cells remain viable. The period of viability of the cells after administration to a subject can be as short as a few hours, e. g. twenty-four hours, to a few days, to as long as several years. In some embodiments, the cells can also be administered (e.g., injected) a location other than the desired site, such as in the brain or subcutaneously, for example, in a capsule to maintain the implanted cells at the implant location and avoid migration of the implanted cells.

Human Leukocyte Antigen and HLA: By “HLA” or “human leukocyte antigen” complex is a gene complex encoding the MHC proteins in humans. These cell-surface proteins that make up the HLA complex are responsible for the regulation of the immune response to antigens. In humans, there are two MHCs, class I and class II, “HLA-I” and “HLA-II”. HLA-I includes three proteins, HLA-A, HLA-B and HLA-C, which present peptides from the inside of the cell, and antigens presented by the HLA-I complex attract killer T-cells (also known as CD8+ T-cells or cytotoxic T cells). The HLA-I proteins are associated with β-2 microglobulin (B2M). HLA-II includes five proteins, HLA-DP, HLA-DM, HLA-DOB, HLA-DQ and HLA-DR, which present antigens from outside the cell to T lymphocytes. This stimulates CD4+ cells (also known as T-helper cells). It should be understood that the use of either “MHC” or “HLA” is not meant to be limiting, as it depends on whether the genes are from humans (HLA) or murine (MHC). Thus, as it relates to mammalian cells, these terms may be used interchangeably herein.

Hypoimmunogenic: As used herein to characterize a cell, the term “hypoimmunogenic” generally means that such cell is less prone to innate or adaptive immune rejection by a subject into which such cells are transplanted, e.g., the cell is less prone to allorejection by a subject into which such cells are transplanted. For example, relative to a cell of the same cell type that does not comprise the modifications, such a hypoimmunogenic cell may be about 2.5%, 5%, 10%, 20%, 30%, 40%, 50%, 60%, 70%, 80%, 90%, 95%, 97.5%, 99% or more less prone to innate or adaptive immune rejection by a subject into which such cells are transplanted. In some embodiments, genome editing technologies are used to modulate the expression of MHC I and MHC II genes, and thus, contribute to generation of a hypoimmunogenic cell. In some embodiments, a hypoimmunogenic cell evades immune rejection in an MHC-mismatched allogeneic recipient. In some instance, differentiated cells produced from the hypoimmunogenic stem cells outlined herein evade immune rejection when administered (e.g., transplanted or grafted) to an MHC-mismatched allogeneic recipient. In some embodiments, a hypoimmunogenic cell is protected from T cell-mediated adaptive immune rejection and/or innate immune cell rejection. Detailed descriptions of hypoimmunogenic cells, methods of producing thereof, and methods of using thereof are found in WO2016183041 filed May 9, 2015; WO2018132783 filed Jan. 14, 2018; WO2018175390 filed Mar. 20, 2018 WO2020018615 filed Jul. 17, 2019; WO2020018620 filed Jul. 17, 2019; PCT/US2020/44635 filed Jul. 31, 2020; WO2021022223 filed Jul. 31, 2020; WO2021041316 filed Aug. 24, 2020; and WO2021222285 filed Apr. 27, 2021, the disclosures including the examples, sequence listings and figures are incorporated herein by reference in their entirety.

Hypoimmunogenicity of a cell can be determined by evaluating the immunogenicity of the cell such as the cell's ability to elicit adaptive and innate immune responses or to avoid eliciting such adaptive and innate immune responses. Such immune response can be measured using assays recognized by those skilled in the art. In some embodiments, an immune response assay measures the effect of a hypoimmunogenic cell on T cell proliferation, T cell activation, T cell killing, donor specific antibody generation, NK cell proliferation, NK cell activation, and macrophage activity. In some cases, hypoimmunogenic cells and derivatives thereof undergo decreased killing by T cells and/or NK cells upon administration to a subject. In some instances, the cells and derivatives thereof show decreased macrophage engulfment compared to an unmodified or wild-type cell. In some embodiments, a hypoimmunogenic cell elicits a reduced or diminished immune response in a recipient subject compared to a corresponding unmodified wild-type cell. In some embodiments, a hypoimmunogenic cell is nonimmunogenic or fails to elicit an immune response in a recipient subject.

Identity: The term percent “identity,” in the context of two or more nucleic acid or polypeptide sequences, refers to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared. For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., infra). One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information.

Immune Signaling Factor: “Immune signaling factor” as used herein refers to, in some cases, a molecule, protein, peptide and the like that activates immune signaling pathways.

Increase, Enhance or Activate: The terms “increase,” “enhance,” or “activate,” as well as grammatical variations thereof, are all used herein to generally mean an increase by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In some embodiments, the reference level, also referred to as the basal level, is 0.

Indel: In some embodiments, the alteration is an indel. As used herein, “indel” refers to a mutation resulting from an insertion, deletion, or a combination thereof. As will be appreciated by those skilled in the art, an indel in a coding region of a genomic sequence will result in a frameshift mutation, unless the length of the indel is a multiple of three. In some embodiments, the alteration is a point mutation. As used herein, “point mutation” refers to a substitution that replaces one of the nucleotides. A gene editing (e.g., CRISPR/Cas) system of the present disclosure can be used to induce an indel of any length or a point mutation in a target polynucleotide sequence.

Knock Down: As used herein, “knock down” refers to a reduction in expression of the target mRNA or the corresponding target protein. Knock down is commonly reported relative to levels present following administration or expression of a noncontrol molecule that does not mediate reduction in expression levels of RNA (e.g., a non-targeting control shRNA, siRNA, or miRNA). In some embodiments, knock down of a target gene is achieved by way of conditional or inducible shRNAs, conditional or inducible siRNAs, conditional or inducible miRNAs, or conditional or inducible CRISPR interference (CRISPRi). In some embodiments, knock down of a target gene is achieved by way of a protein-based method, such as a conditional or inducible degron method. In some embodiments, knock down of a target gene is achieved by genetic modification, including shRNAs, siRNAs, miRNAs, or use of gene editing systems (e.g., CRISPR/Cas).

Knock down is commonly assessed by measuring the mRNA levels using quantitative polymerase chain reaction (qPCR) amplification or by measuring protein levels by western blot or enzyme-linked immunosorbent assay (ELISA). Analyzing the protein level provides an assessment of both mRNA cleavage as well as translation inhibition. Further techniques for measuring knock down include RNA solution hybridization, nuclease protection, northern hybridization, gene expression monitoring with a microarray, antibody binding, radioimmunoassay, and fluorescence activated cell analysis. Those skilled in the art will readily appreciate how to use the gene editing systems (e.g., CRISPR/Cas) of the present disclosure to knock out a target polynucleotide sequence or a portion thereof based upon the details described herein.

Knock Out: As used herein, “knock out” or “knock-out” includes deleting all or a portion of a target polynucleotide sequence in a way that interferes with the translation or function of the target polynucleotide sequence. For example, a knock out can be achieved by altering a target polynucleotide sequence by inducing an insertion or a deletion (“indel”) in the target polynucleotide sequence, including in a functional domain of the target polynucleotide sequence (e.g., a DNA binding domain). Those skilled in the art will readily appreciate how to use the gene editing systems (e.g., CRISPR/Cas) of the present disclosure to knock out a target polynucleotide sequence or a portion thereof based upon the details described herein.

In some embodiments, a genetic modification or alteration results in a knock out or knock down of the target polynucleotide sequence or a portion thereof. Knocking out a target polynucleotide sequence or a portion thereof using a gene editing system (e.g., CRISPR/Cas) of the present disclosure can be useful for a variety of applications. For example, knocking out a target polynucleotide sequence in a cell can be performed in vitro for research purposes. For ex vivo purposes, knocking out a target polynucleotide sequence in a cell can be useful for treating or preventing a disorder associated with expression of the target polynucleotide sequence (e.g., by knocking out a mutant allele in a cell ex vivo and introducing those cells comprising the knocked out mutant allele into a subject) or for changing the genotype or phenotype of a cell.

Knock In: By “knock in” or “knock-in” herein is meant a genetic modification resulting from the insertion of a DNA sequence into a chromosomal locus in a host cell. This causes initiation of or increased levels of expression of the knocked in gene, portion of gene, or nucleic acid sequence inserted product, e.g., an increase in RNA transcript levels and/or encoded protein levels. As will be appreciated by those in the art, this can be accomplished in several ways, including inserting or adding one or more additional copies of the gene or portion thereof to the host cell or altering a regulatory component of the endogenous gene increasing expression of the protein is made or inserting a specific nucleic acid sequence whose expression is desired. This may be accomplished by modifying a promoter, adding a different promoter, adding an enhancer, adding other regulatory elements, or modifying other gene expression sequences.

Modulation: “Modulation” of gene expression refers to a change in the expression level of a gene. Modulation of expression can include, but is not limited to, gene activation and gene repression. Modulation may also be complete, i.e., wherein gene expression is totally inactivated or is activated to wild-type levels or beyond; or it may be partial, wherein gene expression is partially reduced, or partially activated to some fraction of wild-type levels.

Mutant Cell: As used herein, a “mutant cell” refers to a cell with a resulting genotype that differs from its original genotype. In some instances, a “mutant cell” exhibits a mutant phenotype, for example when a normally functioning gene is altered using the gene editing systems (e.g., CRISPR/Cas) systems of the present disclosure. In other instances, a “mutant cell” exhibits a wild-type phenotype, for example when a gene editing system (e.g., CRISPR/Cas) system of the present disclosure is used to correct a mutant genotype. In some embodiments, the target polynucleotide sequence in a cell is altered to correct or repair a genetic mutation (e.g., to restore a normal phenotype to the cell). In some embodiments, the target polynucleotide sequence in a cell is altered to induce a genetic mutation (e.g., to disrupt the function of a gene or genomic element).

Native Cell: The term “native cell” as used herein refers to a cell that is not otherwise modified (e.g., engineered). In some embodiments, a native cell is a naturally occurring wild-type or a control cell.

Operatively Linked or Operably Linked: The term “operatively linked” or “operably linked” are used interchangeably with reference to a juxtaposition of two or more components (such as sequence elements), in which the components are arranged such that both components function normally and allow the possibility that at least one of the components can mediate a function that is exerted upon at least one of the other components. By way of illustration, a transcriptional regulatory sequence, such as a promoter, is operatively linked to a coding sequence if the transcriptional regulatory sequence controls the level of transcription of the coding sequence in response to the presence or absence of one or more transcriptional regulatory factors. A transcriptional regulatory sequence is generally operatively linked in cis with a coding sequence, but need not be directly adjacent to it. For example, an enhancer is a transcriptional regulatory sequence that is operatively linked to a coding sequence, even though they are not contiguous.

Patient: The term “patient” refers to an animal, for example, a human to whom treatment, including prophylactic treatment, with the cells as described herein, is provided. For treatment of those infections, conditions or disease states, which are specific for a specific animal such as a human patient, the term patient refers to that specific animal. The term “patient” also encompasses any vertebrate including but not limited to mammals, reptiles, amphibians and fish. However, advantageously, the patient is a mammal such as a human, or other mammals such as a domesticated mammal, e.g., dog, cat, horse, and the like, or production mammal, e.g., cow, sheep, pig, and the like.

Progeny: As used herein, the term “progeny” encompasses, e.g., a first-generation progeny, i.e., the progeny is directly derived from, obtained from, obtainable from or derivable from the initial iPSC by, e.g., traditional propagation methods. The term “progeny” also encompasses further generations such as second, third, fourth, fifth, sixth, seventh, or more generations, i.e., generations of cells which are derived from, obtained from, obtainable from or derivable from the former generation by, e.g., traditional propagation methods. The term “progeny” also encompasses modified cells that result from the modification or alteration of the initial iPSC or a progeny thereof.

Pluripotent stem cells: “Pluripotent stem cells” as used herein have the potential to differentiate into any of the three germ layers: endoderm (e.g., the stomach linking, gastrointestinal tract, lungs, etc.), mesoderm (e.g., muscle, bone, blood, urogenital tissue, etc.) or ectoderm (e.g., epidermal tissues and nervous system tissues). The term “pluripotent stem cells,” as used herein, also encompasses “induced pluripotent stem cells”, or “iPSCs”, or a type of pluripotent stem cell derived from a non-pluripotent cell. In some embodiments, a pluripotent stem cell is produced or generated from a cell that is not a pluripotent cell. In other words, pluripotent stem cells can be direct or indirect progeny of a non-pluripotent cell. Examples of parent cells include somatic cells that have been reprogrammed to induce a pluripotent, undifferentiated phenotype by various means. Such “iPS” or “iPSC” cells can be created by inducing the expression of certain regulatory genes or by the exogenous application of certain proteins. Methods for the induction of iPS cells are known in the art and are further described below. (See, e.g., Zhou et al., Stem Cells 27 (11): 2667-74 (2009); Huangfu et al., Nature Biotechnol. 26 (7): 795 (2008); Woltjen et al., Nature 458 (7239): 766-770 (2009); and Zhou et al., Cell Stem Cell 8:381-384 (2009); each of which is incorporated by reference herein in their entirety.) The generation of induced pluripotent stem cells (iPSCs) is outlined below. As used herein, “hiPSCs” are human induced pluripotent stem cells. In some embodiments, “pluripotent stem cells,” as used herein, also encompasses mesenchymal stem cells (MSCs), and/or embryonic stem cells (ESCs).

Promoter: As used herein, “promoter,” “promoter sequence,” or “promoter region” refers to a DNA regulatory region/sequence capable of binding RNA polymerase and involved in initiating transcription of a downstream coding or non-coding sequence. In some examples, the promoter sequence includes the transcription initiation site and extends upstream to include the minimum number of bases or elements necessary to initiate transcription at levels detectable above background. In some embodiments, the promoter sequence includes a transcription initiation site, as well as protein binding domains responsible for the binding of RNA polymerase. Eukaryotic promoters will often, but not always, contain “TATA” boxes and “CAT” boxes.

Propagated from a Primary T cell or a Progeny Thereof: In some embodiments, the engineered and hypoimmunogenic cells described are propagated from a primary T cell or a progeny thereof. As used herein, the term “propagated from a primary T cell or a progeny thereof” encompasses the initial primary T cell that is isolated from the donor subject and any subsequent progeny thereof.

Regulatory elements: As used herein, the terms “regulatory sequences,” “regulatory elements,” and “control elements” are interchangeable and refer to polynucleotide sequences that are upstream (5′ non-coding sequences), within, or downstream (3′ non-translated sequences) of a polynucleotide target to be expressed. Regulatory sequences influence, for example but are not limited to, the timing of transcription, amount or level of transcription, RNA processing or stability, and/or translation of the related structural nucleotide sequence. Regulatory sequences may include activator binding sequences, enhancers, introns, polyadenylation recognition sequences, promoters, repressor binding sequences, stem-loop structures, translational initiation sequences, translation leader sequences, transcription termination sequences, translation termination sequences, primer binding sites, and the like. It is recognized that since in most cases the exact boundaries of regulatory sequences have not been completely defined, nucleotide sequences of different lengths may have identical regulatory or promoter activity.

Safe harbor locus: “Safe harbor locus” as used herein refers to a gene locus that allows expression of a transgene or an exogenous gene in a manner that enables the newly inserted genetic elements to function predictably and that also may not cause alterations of the host genome in a manner that poses a risk to the host cell. Exemplary “safe harbor” loci include, but are not limited to, a CCR5 gene, a PPP1R12C (also known as AAVS1) gene, a CLYBL gene, and/or a Rosa gene (e.g., ROSA26).

Safety Switch: In some embodiments, engineered cells disclosed herein comprise a safety switch. The term “safety switch” used herein refers to a system for controlling the expression of a gene or protein of interest that, when downregulated or upregulated, leads to clearance or death of the cell, e.g., through recognition by the host's immune system. A safety switch can be designed to be triggered by an exogenous molecule in case of an adverse clinical event. A safety switch can be engineered by regulating the expression on the DNA, RNA and protein levels. A safety switch includes a protein or molecule that allows for the control of cellular activity in response to an adverse event. In one embodiment, the safety switch is a “kill switch” that is expressed in an inactive state and is fatal to a cell expressing the safety switch upon activation of the switch by a selective, externally provided agent. In one embodiment, the safety switch gene is cis-acting in relation to the gene of interest in a construct. Activation of the safety switch causes the cell to kill solely itself or itself and neighboring cells through apoptosis or necrosis. In some embodiments, the cells disclosed herein, e.g., stem cells, induced pluripotent stem cells, hematopoietic stem cells, primary cells, or differentiated cell, including, but not limited to, cardiac cells, cardiac progenitor cells, neural cells, glial progenitor cells, endothelial cells, T cells, B cells, pancreatic islet cells including pancreatic beta islet cells, retinal pigmented epithelium cells, hepatocytes, thyroid cells, skin cells, blood cells, plasma cells, platelets, renal cells, epithelial cells, CART cells, NK cells, and/or CAR-NK cells, comprise a safety switch.

Suicide Gene: In some embodiments, the cells disclosed herein comprise a “suicide gene” (or “suicide switch”). The suicide gene can cause the death of the hypoimmunogenic cells should they grow and divide in an undesired manner. The suicide gene ablation approach includes a suicide gene in a gene transfer vector encoding a protein that results in cell killing only when activated by a specific compound. A suicide gene can encode an enzyme that selectively converts a nontoxic compound into highly toxic metabolites. In some embodiments, the cells disclosed herein, e.g., stem cells, induced pluripotent stem cells, hematopoietic stem cells, primary cells, or differentiated cell, including, but not limited to, cardiac cells, cardiac progenitor cells, neural cells, glial progenitor cells, endothelial cells, T cells, B cells, pancreatic islet cells including pancreatic beta islet cells, retinal pigmented epithelium cells, hepatocytes, thyroid cells, skin cells, blood cells, plasma cells, platelets, renal cells, epithelial cells, CART cells, NK cells, and/or CAR-NK cells, comprise a suicide gene.

Suspected of: The term “suspected of” as used herein refers to a situation in which one or more indicators, signs, or symptoms indicate that a condition may be occurring or is occurring or that a condition has occurred. For example, if a patient is suspected of having antigen evasion (e.g., some cells of the patient have reduced or lost expression of an antigen), it means that one or more indicators, signs, or symptoms indicate that antigen evasion may be occurring or is occurring or that antigen evasion has occurred. In some embodiments, an indicator, sign, or symptom of antigen evasion comprises a disease or disorder a patient has, how long a patient has had or been at risk of having a disease or disorder, loss of responsiveness to one or more targeted therapies, progressive worsening of a disease or disorder (e.g., demonstrated by increased tumor burden, increased growth of tumor cells, tumor mass, or number of tumors), demographics of a patient (e.g., a patient's age, a patient's sex, a patient's weight, a patient's BMI), a presence of certain biomarkers, an alteration in a level of certain biomarkers, etc.

Target locus: “Target locus” as used herein refers to a gene locus that allows expression of a transgene or an exogenous gene. Exemplary “target loci” include, but are not limited to, a CXCR4 gene, an albumin gene, a SHS231 locus, an F3 gene (also known as CD142), a MICA gene, a MICB gene, a LRP1 gene (also known as CD91), a HMGB1 gene, an ABO gene, a RHD gene, a FUT1 gene, and/or a KDM5D gene (also known as HY). The exogenous polynucleotide encoding the exogenous gene can be inserted in the CDS region for B2M, CIITA, TRAC, TRBC, CCR5, F3 (i.e., CD142), MICA, MICB, LRP1, HMGB1, ABO, RHD, FUT1, KDM5D (i.e., HY), PDGFRa, OLIG2, and/or GFAP. The exogenous polynucleotide encoding the exogenous gene can be inserted in introns 1 or 2 for PPP1R12C (i.e., AAVS1) or CCR5. The exogenous polynucleotide encoding the exogenous gene can be inserted in exons 1 or 2 or 3 for CCR5. The exogenous polynucleotide encoding the exogenous gene can be inserted in intron 2 for CLYBL. The exogenous polynucleotide encoding the exogenous gene can be inserted in a 500 bp window in Ch-4:58,976,613 (i.e., SHS231). The exogenous polynucleotide encoding the exogenous gene can be insert in any suitable region of the aforementioned safe harbor or target loci that allows for expression of the exogenous gene, including, for example, an intron, an exon or a coding sequence region in a safe harbor or target locus.

Target: As used herein, a “target” can refer to a gene, a portion of a gene, a portion of the genome, or a protein that is subject to regulatable reduced expression by the methods described herein. A target can also be an antigen to which a therapeutic agent or targeted therapy is directed.

Therapeutically Effective Amount: As used herein, “therapeutically effective amount” refers to an amount sufficient to provide a therapeutic benefit in the treatment and/or management of a disease, disorder, or condition. In some embodiments, a therapeutically effective amount is an amount sufficient to ameliorate, palliate, stabilize, reverse, slow, attenuate or delay the progression of a disease, disorder, or condition, or of a symptom or side effect of the disease, disorder, or condition. In some embodiments, the therapeutically effective amount is also a clinically effective amount. In other embodiments, the therapeutically effective amount is not a clinically effective amount.

Tolerogenicfactor: “Tolerogenic factor,” “immunosuppressive factor,” or “immune regulatory factor” as used herein include hypoimmunity factors, complement inhibitors, and other factors that modulate or affect the ability of a cell to be recognized by the immune system of a host or recipient subject upon administration, transplantation, or engraftment. These may be in combination with additional genetic modifications. In some embodiments, a tolerogenic factor is or comprises A20/TNFAIP3, C1-Inhibitor, CCL21, CCL22, CD16, CD16 Fc receptor, CD24, CD27, CD35, CD39, CD46, CD47, CD52, CD55, CD59, CD64, CD200, CR1, CTLA4-Ig, DUX4, FasL, H2-M3, HLA-C, HLA-E, HLA-E heavy chain, HLA-F, HLA-G, IDO1, IL-10, IL15-RF, IL-35, IL-39, MANF, Mfge8, PD-L1, Serpinb9, CCL21, CCL22, B2M-HLA-E, C1 inhibitor, or CR1.

Treat: As used herein, the terms “treat,” “treating” and “treatment” includes administering to a subject a therapeutically or clinically effective amount of cells described herein so that the subject has a reduction in at least one symptom of the disease or an improvement in the disease, for example, beneficial or desired therapeutic or clinical results. For purposes of this technology, beneficial or desired therapeutic or clinical results include, but are not limited to, alleviation of one or more symptoms, diminishment of extent of disease, stabilized (i.e., not worsening) state of disease, delay or slowing of disease progression, amelioration or palliation of the disease state, and remission (whether partial or total), whether detectable or undetectable. Treating can refer to prolonging survival as compared to expected survival if not receiving treatment. Thus, one of skill in the art realizes that a treatment may improve the disease condition, but may not be a complete cure for the disease. In some embodiments, one or more symptoms of a condition, disease or disorder are alleviated by at least 5%, at least 10%, at least 20%, at least 30%, at least 40%, or at least 50% upon treatment of the condition, disease or disorder. In some embodiments, beneficial or desired therapeutic or clinical results of disease treatment include, but are not limited to, alleviation of one or more symptoms, diminishment of extent of disease, stabilized (i.e., not worsening) state of disease, delay or slowing of disease progression, amelioration or palliation of the disease state, and remission (whether partial or total), whether detectable or undetectable.

Vector: A “vector” or “construct” is capable of transferring gene sequences to target cells. Typically, “vector construct,” “expression vector,” and “gene transfer vector,” mean any nucleic acid construct capable of directing the expression of a gene of interest and which can transfer gene sequences to target cells. Thus, the term includes cloning, and expression vehicles, as well as integrating vectors. Methods for the introduction of vectors or constructs into cells are known to those of skill in the art and include, but are not limited to, lipid-mediated transfer (i.e., liposomes, including neutral and cationic lipids), electroporation, direct injection, cell fusion, particle bombardment, calcium phosphate co-precipitation, DEAE-dextran-mediated transfer and/or viral vector-mediated transfer.

Wild-Type: By “wild-type” or “wt” or “control” in the context of a cell means any cell found in nature. Examples of wild type or control cells include primary cells and T cells found in nature. However, in some embodiments, the cells are engineered to have reduced or increased expression of one or more targets relative to an unaltered or unmodified wild-type cell. In some embodiments, the cells are engineered to have constitutive reduced or increased expression of one or more targets relative to an unaltered or unmodified wild-type cell. In some embodiments, the cells are engineered to have regulatable reduced or increased expression of one or more targets relative to an unaltered or unmodified wild-type cell. In some embodiments, the cells comprise increased expression of CD47 relative to a wild-type cell or a control cell of the same cell type. By way of example, in the context of an engineered cell, as used herein, “wild-type” or “control” can also mean an engineered cell that may contain nucleic acid changes resulting in reduced expression of MHC I and/or 11 and/or T-cell receptors, but did not undergo the gene editing procedures to result in overexpression of CD47 proteins. For example, as used herein, “wild-type” or “control” means an engineered cell that comprises reduced or knocked out expression of B2M, CIITA, and/or TRAC. Also as used herein, “wild-type” or “control” means an engineered cell that comprises reduced or knocked out expression of B2M, CIITA, TRAC, and/or TRBC. As used herein, “wild-type” or “control” also means an engineered cell that may contain nucleic acid changes resulting in overexpression of CD47 proteins, but did not undergo the gene editing procedures to result in reduced expression of MHC I and/or II and/or T-cell receptors. In the context of an iPSC or a progeny thereof, “wild-type” or “control” also means an iPSC or progeny thereof that may contain nucleic acid changes resulting in pluripotency but did not undergo the gene editing procedures of the present disclosure to achieve reduced expression of MHC I and/or 11 and/or T-cell receptors, and/or overexpression of CD47 proteins. For example, as used herein, “wild-type” or “control” means an iPSC or progeny thereof that comprises reduced or knocked out expression of B2M, CIITA, and/or TRAC. Also as used herein, “wild-type” or “control” means an iPSC or progeny thereof that comprises reduced or knocked out expression of B2M, CIITA, TRAC, and/or TRBC. In the context of a primary T cell or a progeny thereof, “wild-type” or “control” also means a primary T cell or progeny thereof that may contain nucleic acid changes resulting in reduced expression of MHC I and/or II and/orT-cell receptors, but did not undergo the gene editing procedures to result in overexpression of CD47 proteins. For example, as used herein, “wild-type” or “control” means a primary T cell or progeny thereof that comprises reduced or knocked out expression of B2M, CIITA, and/or TRAC. Also as used herein, “wild-type” or “control” means a primary T cell or progeny thereof that comprises reduced or knocked out expression of B2M, CIITA, TRAC, and/or TRBC. Also in the context of a primary T cell or a progeny thereof, “wild-type” or “control” also means a primary T cell or progeny thereof that may contain nucleic acid changes resulting in overexpression of CD47 proteins, but did not undergo the gene editing procedures to result in reduced expression of MHC I and/or 11 and/or T-cell receptors. In some embodiments, the cells are engineered to have regulatable reduced or increased expression of one or more targets relative to a cell of the same cell type that does not comprise the modifications. In some embodiments, the wild-type cell or the control cell is a starting material. In some embodiments, the starting material is a primary cell collected from a donor. In some embodiments, the starting material is a primary blood cell collected from a donor, e.g., via a leukopak. For example, unmodified T cells obtained from a donor is a starting material that are considered wild-type or control cells as contemplated herein. In another example, an iPSC cell line starting material is a starting material that is considered a wild-type or control cell as contemplated herein. In some embodiments, the starting material is otherwise modified or engineered to have altered expression of one or more genes to generate the engineered cell.

It is noted that the claims may be drafted to exclude any optional element. As such, this statement is intended to serve as antecedent basis for use of such exclusive terminology as “solely,” “only,” and the like in connection with the recitation of claim elements, or use of a “negative” limitation. As will be apparent to those of skill in the art upon reading this disclosure, each of the individual embodiments described and illustrated herein has discrete components and features readily separated from or combined with the features of any of the other several embodiments without departing from the scope or spirit of the present disclosure. Any recited method may be carried out in the order of events recited or in any other order that is logically possible. Although any methods and materials similar or equivalent to those described herein may also be used in the practice or testing of the present disclosure, representative illustrative methods and materials are now described.

Unless defined otherwise, all technical and scientific terms used herein have the same meaning as commonly understood by one of ordinary skill in the art to which this technology belongs. Where a range of values is provided, it is understood that each intervening value, to the tenth of the unit of the lower limit unless the context clearly dictates otherwise, between the upper and lower limit of that range and any other stated or intervening value in that stated range, is encompassed within the present disclosure. The upper and lower limits of these smaller ranges may independently be included in the smaller ranges and are also encompassed within the present disclosure, subject to any specifically excluded limit in the stated range. Where the stated range includes one or both of the limits, ranges excluding either or both of those included limits are also included in the present disclosure. Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number, which, in the context presented, provides the substantial equivalent of the specifically recited number. The term about is used herein to mean plus or minus ten percent (10%) of a value. For example, “about 100” refers to any number between 90 and 110.

All publications, patents, and patent applications cited in this specification are incorporated herein by reference to the same extent as if each individual publication, patent, or patent application were specifically and individually indicated to be incorporated by reference. Furthermore, each cited publication, patent, or patent application is incorporated herein by reference to disclose and describe the subject matter in connection with which the publications are cited. The citation of any publication is for its disclosure prior to the filing date and should not be construed as an admission that the technology described herein is not entitled to antedate such publication by virtue of prior technology. Further, the dates of publication provided might be different from the actual publication dates, which may need to be independently confirmed.

Before the technology is further described, it is to be understood that this technology is not limited to particular embodiments described, as such may, of course, vary. It is also to be understood that the terminology used herein is for the purpose of describing particular embodiments only, and is not intended to be limiting, since the scope of the present disclosure will be limited only by the appended claims. It should also be understood that the headers used herein are not limiting and are merely intended to orient the reader, but the subject matter generally applies to the technology disclosed herein.

### BRIEF DESCRIPTION OF THE DRAWING

FIG. 1 depicts an exemplary timeline and experimental setup for assessing the efficacy of CAR-T cells. Specifically, FIG. 1 depicts an exemplary timeline and experimental setup for testing the efficacy of CD19 CAR-T cells, CD22 CAR-T cells, and CD19×CD22 CAR-T cells in an NSG mouse model inoculated with 70%:30% mixture of Nalm6:Nalm6-CD19KO tumor cells as an antigen escape model.

FIG. 2 includes a table summarizing mice and experimental conditions used to test the efficacy of CD19 CAR-T cells, CD22 CAR-T cells, and CD19×CD22 CAR-T cells in an NSG mouse model inoculated with 70%:30% mixture of Nalm6:Nalm6-CD19KO tumor cells as an antigen escape model.

FIG. 3 includes a line graph showing bioluminescence measurements at select time points from NSG mice inoculated with 70%:30% mixture of Nalm6:Nalm6-CD19KO tumor cells and administered CD19 CAR-T cells, CD22 CAR-T cells, or CD19×CD22 CAR-T cells derived from a first donor (Donor 1). Bioluminescence measurements at select time points from NSG mice serving as controls are also included.

FIG. 4 depicts includes a line graph showing bioluminescence measurements at select time points from NSG mice inoculated with 70%:30% mixture of Nalm6:Nalm6-CD19KO tumor cells and administered CD19 CAR-T cells, CD22 CAR-T cells, or CD19×CD22 CAR-T cells derived from a second donor (Donor 2). Bioluminescence measurements at select time points from NSG mice serving as controls are also included.

FIG. 5 depicts in vivo bioluminescent imaging scans obtained from NSG mice inoculated with 70%:30% mixture of Nalm6:Nalm6-CD19KO tumor cells and administered CD19 CAR-T cells, CD22 CAR-T cells, or CD19×CD22 CAR-T cells derived from Donor 1 and Donor 2.

FIG. 6 depicts an exemplary timeline and experimental setup for assessing the efficacy of CAR-T cells. Specifically, FIG. 6 depicts an exemplary timeline and experimental setup for testing the efficacy of CD19 CAR-T cells, CD22 CAR-T cells, and CD19×CD22 CAR-T cells in an NSG mouse model inoculated with 70%:30% mixture of RAJI:RAJI-CD19KO tumor cells as an antigen escape model.

FIG. 7 includes a table summarizing mice and experimental conditions used to test the efficacy of CD19 CAR-T cells, CD22 CAR-T cells, and CD19×CD22 CAR-T cells in an NSG mouse model inoculated with 70%:30% mixture of RAJI:RAJI-CD19KO tumor cells as an antigen escape model.

FIG. 8 includes a line graph showing bioluminescence measurements at select time points from NSG mice inoculated with 70%:30% mixture of RAJI:RAJI-CD19KO tumor cells and administered CD19 CAR-T cells, CD22 CAR-T cells, or CD19×CD22 CAR-T cells derived from Donor 1. Bioluminescence measurements at select time points from NSG mice serving as controls are also included.

FIG. 9 includes a line graph showing bioluminescence measurements at select time points from NSG mice inoculated with 70%:30% mixture of RAJI:RAJI-CD19KO tumor cells and administered CD19 CAR-T cells, CD22 CAR-T cells, or CD19×CD22 CAR-T cells derived from Donor 2. Bioluminescence measurements at select time points from NSG mice serving as controls are also included.

FIG. 10 depicts in vivo bioluminescent imaging scans obtained from NSG mice inoculated with 70%:30% mixture of RAJI:RAJI-CD19KO tumor cells and administered CD19 CAR-T cells, CD22 CAR-T cells, or CD19×CD22 CAR-T cells derived from Donor 1 and Donor 2.

FIG. 11 depicts an exemplary timeline and experimental setup for assessing the efficacy of CAR-T cells. Specifically, FIG. 11 depicts an exemplary timeline and experimental setup for testing the antitumor activity of dual transduced CD19 CAR×CD22 CAR-T cells (or dual transduced and sorted CD19 CAR×CD22 CAR-T cells) versus the antitumor activity of a combined product of single transduced CD19 CAR-T cells and single transduced and CD22 CAR-T cells in mice that have received Nalm6 tumor cells.

FIG. 12 depicts includes a table summarizing mice and experimental conditions used to test the antitumor activity of dual transduced CD19 CAR×CD22 CAR-T cells (or dual transduced and sorted CD19 CAR×CD22 CAR-T cells) versus the antitumor activity of a combined product of single transduced CD19 CAR-T cells and single transduced and CD22 CAR-T cells.

FIG. 13 includes a line graph showing bioluminescence measurements at select time points from NSG mice inoculated with Nalm6 tumor cells and administered CD19 CAR-T cells, CD22 CAR-T cells, or CD19×CD22 CAR-T cells derived from Donor 2. Bioluminescence measurements at select time points from NSG mice serving as controls are also included.

FIG. 14 includes schematics representing a therapeutic agent comprising an exemplary population of engineered cells (e.g., engineered CAR-T cells) as provided herein. FIG. 14A includes a schematic representing a therapeutic agent comprising a first population of engineered cells comprising a first CAR. FIG. 14B includes a schematic representing a therapeutic agent comprising a first population of engineered cells comprising a second CAR. FIG. 14C includes a schematic representing a therapeutic agent comprising a first population of engineered cells comprising a first CAR and a second CAR.

FIG. 15 includes schematics representing a therapeutic agent comprising two exemplary populations of engineered cells (e.g., engineered CAR-T cells) as provided herein. FIG. 15A includes a schematic representing a therapeutic agent comprising a first population of engineered cells comprising a first CAR and a second population of engineered cells comprising a second CAR. FIG. 15B includes a schematic representing a therapeutic agent comprising a first population of engineered cells comprising a first CAR and a second population of engineered cells comprising a first CAR and a second CAR. FIG. 15C includes a schematic representing a therapeutic agent comprising a first population of engineered cells comprising a second CAR and a second population of engineered cells comprising a first CAR and a second CAR.

FIG. 16 includes a schematic representing a therapeutic agent comprising three exemplary populations of engineered cells (e.g., engineered CAR-T cells) as provided herein. In particular, FIG. 16 includes a schematic representing a therapeutic agent comprising a first population of engineered cells comprising a first CAR, a second population of engineered cells comprising a second CAR, and a third population of engineered cells comprising a first CAR and a second CAR.

### DETAILED DESCRIPTION

Among other things, the present disclosure provides the methods for treating patients who are at risk of or experiencing antigen evasion or antigenic drift. Also provided are methods of treating patients who have a disease or disorder that is associated with, characterized by, or prone to antigen evasion or antigenic drift. An exemplary disease is cancer, e.g., B cell malignancies.

Further, provided herein are engineered cells that can be used in methods provided herein. Thus, in some embodiments, escribed herein are engineered or modified immune evasive cells based, in part, on the hypoimmune editing platform described in WO2018132783, including but not limited to human immune evasive cells. To overcome the problem of a subject's immune rejection of these primary and/or stem cell-derived transplants, hypoimmunogenic cells (e.g., hypoimmunogenic pluripotent cells, differentiated cells derived from such, and primary cells) described herein represent a viable source for any transplantable cell type. Such cells are protected from adaptive and/or innate immune rejection upon administration to a recipient subject. Advantageously, the cells disclosed herein are not rejected by the recipient subject's immune system, regardless of the subject's genetic make-up, as they are protected from adaptive and innate immune rejection upon administration to a recipient subject. In some embodiments, the engineered and/or hypoimmunogenic cells do not express major histocompatibility complex (MHC) class I and class II antigens and/or T-cell receptors. In certain embodiments, the engineered and/or hypoimmunogenic cells do not express MHC I and II antigens and/or T-cell receptors and overexpress CD47 proteins. In certain embodiments, the engineered and/or hypoimmunogenic cells such as engineered and/or hypoimmunogenic T cells do not express MHC I and II antigens and/or T-cell receptors, overexpress CD47 proteins and express exogenous CARs.

In some embodiments, hypoimmunogenic cells outlined herein are not subject to an innate immune cell rejection. In some instances, hypoimmunogenic cells are not susceptible to NK cell-mediated lysis. In some instances, hypoimmunogenic cells are not susceptible to macrophage engulfment. In some embodiments, hypoimmunogenic cells are useful as a source of universally compatible cells or tissues (e.g., universal donor cells or tissues) that are transplanted into a recipient subject with little to no immunosuppressant agent needed. Such hypoimmunogenic cells retain cell-specific characteristics and features upon transplantation, including, e.g., pluripotency, as well as being capable of engraftment and functioning similarly to a corresponding native cell.

The technology disclosed herein utilizes expression of tolerogenic factors and modulation (e.g., reduction or elimination) of MHC I, MHC II, and/or TCR expression in human cells. In some embodiments, genome editing technologies utilizing rare-cutting endonucleases (e.g., the CRISPR/Cas, TALEN, zinc finger nuclease, meganuclease, and homing endonuclease systems) are also used to reduce or eliminate expression of genes involved in an immune response (e.g., by deleting genomic DNA of genes involved in an immune response or by insertions of genomic DNA into such genes, such that gene expression is impacted) in the cells. In some embodiments, genome editing technologies or other gene modulation technologies are used to insert tolerance-inducing (tolerogenic) factors in human cells, rendering the cells and their progeny (include any differentiated cells prepared therefrom) able to evade immune recognition upon engrafting into a recipient subject. As such, the cells described herein exhibit modulated expression of one or more genes and factors that affect MHC I, MHC II, and/or TCR expression and evade the recipient subject's immune system.

The genome editing techniques enable double-strand DNA breaks at desired locus sites. These controlled double-strand breaks promote homologous recombination at the specific locus sites. This process focuses on targeting specific sequences of nucleic acid molecules, such as chromosomes, with endonucleases that recognize and bind to the sequences and induce a double-stranded break in the nucleic acid molecule. The double-strand break is repaired either by an error-prone non-homologous end-joining (NHEJ) or by homologous recombination (HR).

The practice of the numerous embodiments will employ, unless indicated specifically to the contrary, conventional methods of chemistry, biochemistry, organic chemistry, molecular biology, microbiology, recombinant DNA techniques, genetics, immunology, and cell biology that are within the skill of the art, many of which are described below for the purpose of illustration. Such techniques are explained fully in the literature. See, e.g., Sambrook, et al., Molecular Cloning: A Laboratory Manual (3rd Edition, 2001); Sambrook, et al., Molecular Cloning: A Laboratory Manual (2nd Edition, 1989); Maniatis et al., Molecular Cloning: A Laboratory Manual (1982); Ausubel et al., Current Protocols in Molecular Biology (John Wiley and Sons, updated July 2008); Short Protocols in Molecular Biology: A Compendium of Methods from Current Protocols in Molecular Biology, Greene Pub. Associates and Wiley-Interscience; Glover, DNA Cloning: A Practical Approach, vol. I & II (IRL Press, Oxford, 1985); Anand, Techniques for the Analysis of Complex Genomes, (Academic Press, New York, 1992); Transcription and Translation (B. Hames & S. Higgins, Eds., 1984); Perbal, A Practical Guide to Molecular Cloning (1984); Harlow and Lane, Antibodies, (Cold Spring Harbor Laboratory Press, Cold Spring Harbor, N.Y., 1998) Current Protocols in Immunology Q. E. Coligan, A. M. Kruisbeek, D. H. Margulies, E. M. Shevach and W. Strober, eds., 1991); Annual Review of Immunology; as well as monographs in journals such as Advances in Immunology.

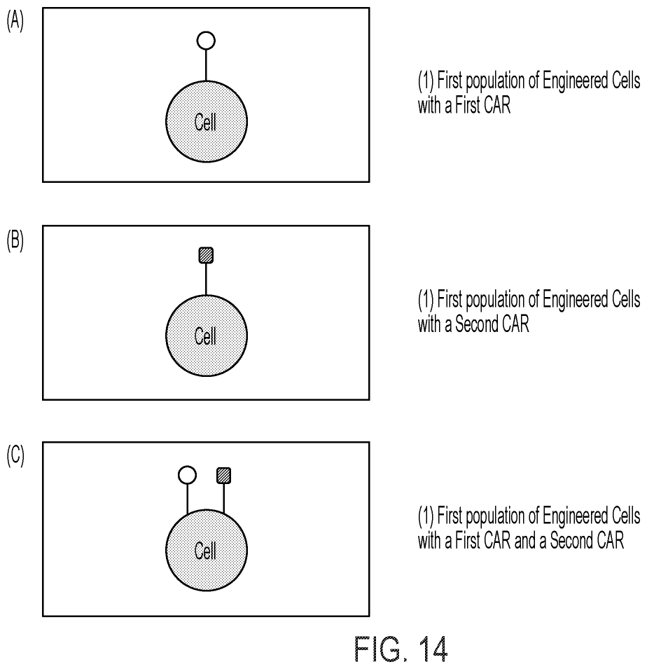

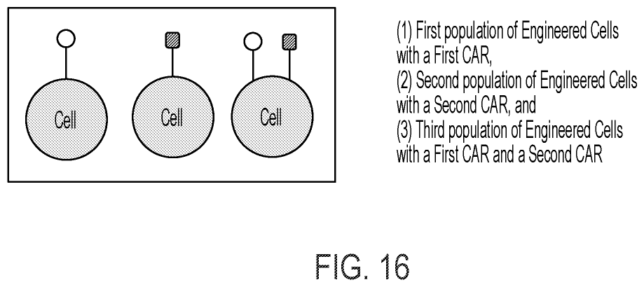

Compositions (e.g., therapeutic agents) comprising engineered cells as described herein are also provided. FIGS. 14-16 show schematics representing exemplary compositions provided. In some embodiments, compositions (e.g., therapeutic agents) or components thereof are directed to a therapeutic target (e.g., an antigen), where the patient receiving such a composition has not previously been administered a targeted therapy directed to that therapeutic target (e.g., an antigen). In some embodiments, compositions (e.g., therapeutic agents) or components thereof are directed to multiple therapeutic targets (e.g., an antigens), where the patient receiving such a composition has not previously been administered a targeted therapy directed to at least one of the therapeutic targets (e.g., an antigens).

### A. Administering Hypoimmunogenic Cells to Patients

In one aspect provided herein is a method of treating a patient by administering a therapeutic agent (e.g., a population of the engineered CAR-T cells) described herein. A therapeutic agent described herein (e.g., engineered CAR-T cells) provided herein can be administered to any suitable patients including, for example, a candidate for a cellular therapy for the treatment of a disease or disorder. Candidates for cellular therapy include any patient having a disease or condition that may potentially benefit from the therapeutic effects of a therapeutic agent (e.g., engineered CAR-T cells) provided herein. In some embodiments, the patient has a cellular deficiency. A candidate who benefits from the therapeutic effects of a therapeutic agent (e.g., engineered CAR-T cells) provided herein exhibits an elimination, reduction or amelioration of the disease or condition. In some embodiments, the patient administered a therapeutic agent (e.g., engineered CAR-T cells) has a cancer. Exemplary cancers that can be treated by a therapeutic agent (e.g., engineered CAR-T cells) provided herein include, but are not limited to, lymphoma, leukemia, B cell acute lymphoblastic leukemia (B-ALL), diffuse large B-cell lymphoma, B-cell Non-Hodgkin lymphoma (B-NHL), B-cell chronic lymphoblastic leukemia (B-CLL), liver cancer, pancreatic cancer, breast cancer, ovarian cancer, colorectal cancer, lung cancer, non-small cell lung cancer, acute myeloid lymphoid leukemia, multiple myeloma, gastric cancer, gastric adenocarcinoma, pancreatic adenocarcinoma, glioblastoma, neuroblastoma, lung squamous cell carcinoma, hepatocellular carcinoma, and/or bladder cancer. In some embodiments, any of the exemplary cancers are also a CD19-negative cancer, a CD22-positive cancer, a CD19-negative/CD22-positive cancer, or a CD19-positive cancer. In certain embodiments, any of the exemplary cancers underwent antigen evasion and no longer express an antigen or have reduced expression of an antigen previously expressed. For example, any of the exemplary cancers can be a CD19-negative and a CD22-positive cancer but were previously CD19-positive and CD22-negative or CD22-positive. In certain embodiments, the cancer patient is treated by administration of a therapeutic agent (e.g., a hypoimmunogenic cell, e.g., a hypoimmungogenic CAR-T-cell) provided herein.

### 1. Prior Treatments

In some embodiments, the patient undergoing a treatment using a therapeutic agent (e.g., engineered CAR-T cells) provided herein received a previous treatment (e.g., a targeted therapy). In some embodiments, a therapeutic agent (e.g., engineered CAR-T cells) are used to treat the same condition as the previous treatment. In some embodiments, the same condition is characterized by expression of a different antigen when treated with a therapeutic agent (e.g., engineered CAR-T cells) provided herein compared to an antigen expressed when treated with the previous treatment (e.g., targeted therapy). In some embodiments, a therapeutic agent (e.g., engineered CAR-T cells) provided herein are used to treat a different condition from a previous treatment (e.g., targeted therapy). In some embodiments, a therapeutic agent (e.g., engineered CAR-T cells) administered to a patient exhibit an enhanced therapeutic effect for the treatment of the same condition or disease treated by a previous treatment. In some embodiments, a therapeutic agent (e.g., engineer cells, e.g., hypoimmungenic engineered cells, e.g., hypoimmunogenic engineered CAR-T cells) exhibit a longer therapeutic effect for the treatment of a condition, disorder or disease in a patient as compared to a previous treatment (e.g., In exemplary embodiments, a therapeutic agent (e.g., engineer cells, e.g., hypoimmungenic engineered cells, e.g., hypoimmunogenic engineered CAR-T cells) exhibit an enhanced potency, efficacy, and/or specificity against the cancer cells as compared to the previous treatment. In particular embodiments, engineered CAR-T cells are CAR-T-cells for the treatment of a cancer.

In some embodiments, a patient receiving a therapeutic agent (e.g., engineered CAR-T cells) provided herein received a prior treatment. In some embodiments, the prior treatment comprises an antibody-based therapy (e.g., monoclonal antibodies, antibody-drug conjugates, bispecific antibodies), an immune-oncology therapy (e.g., immune checkpoint inhibitors, antibodies, antibody-drug conjugates, CAR-T cells, vaccines, oncolytic viruses), or a cell-based therapy (e.g., CAR-T cells, TCR-T cells, CAR-NK cells, dendritic cells, NK cells, and other cells, e.g., tumor infiltrating lymphocytes, safety-switch modified T cells, virus-activated T cells, gamma delta T cells). In some embodiments, the prior treatment comprises a cell-based therapy comprising an autologous CAR-T therapy or an allogeneic CAR-T therapy. In some embodiments, the prior treatment comprises autologous CAR-T cells expressing a CD22-specific CAR that is the same as the CAR expressed by the engineered CAR-T cells. In some embodiments, the prior treatment comprises autologous CAR-T cells expressing a CD22-specific CAR that is different from the CAR expressed by the engineered CAR-T cells. In some embodiments, the prior treatment comprises allogeneic CAR-T cells expressing a CD22-specific CAR that is the same as the CAR expressed by the engineered CAR-T cells. In some embodiments, the prior treatment comprises allogeneic CAR-T cells expressing a CD22-specific CAR that is different from the CAR expressed by the engineered CAR-T cells. In some embodiments, the prior treatment comprises autologous CAR-T cells expressing a CAR that is different from the CAR expressed by the engineered CAR-T cells. In some embodiments, the prior treatment comprises allogeneic CAR-T cells expressing a CAR that is different from the CAR expressed by the engineered CAR-T cells. In some embodiments, the prior treatment comprises autologous CAR-T cells expressing a CD19-specific CAR. In some embodiments, the prior treatment comprises allogeneic CAR-T cells expressing a CD19-specific CAR. In some embodiments, the prior treatment comprises axicabtagene ciloleucel, lisocabtagene maraleucel, brexucabtagene autoleucel, or tisagenlecleucel.

In some embodiments, the prior treatment comprised an antibody-based therapy, an immune-oncology therapy, or a cell-based therapy. In some embodiments, the prior treatment comprised a cell-based therapy comprising an autologous CAR-T therapy or an allogeneic CAR-T therapy. In some embodiments, the prior treatment comprised autologous CAR-T cells expressing a CD22-specific CAR that is the same as the CAR expressed by the engineered CAR-T cells. In some embodiments, the prior treatment comprised autologous CAR-T cells expressing a CD22-specific CAR that is different from the CAR expressed by the engineered CAR-T cells. In some embodiments, the prior treatment comprised allogeneic CAR-T cells expressing a CD22-specific CAR that is the same as the CAR expressed by the engineered CAR-T cells. In some embodiments, the prior treatment comprised allogeneic CAR-T cells expressing a CD22-specific CAR that is different from the CAR expressed by the engineered CAR-T cells. In some embodiments, the prior treatment comprised autologous CAR-T cells expressing a CAR that is different from the CAR expressed by the engineered CAR-T cells. In some embodiments, the prior treatment comprised allogeneic CAR-T cells expressing a CAR that is different from the CAR expressed by the engineered CAR-T cells. In some embodiments, the prior treatment comprised autologous CAR-T cells expressing a CD19-specific CAR. In some embodiments, the prior treatment comprised allogeneic CAR-T cells expressing a CD19-specific CAR. In some embodiments, the prior treatment comprised axicabtagene ciloleucel, lisocabtagene maraleucel, brexucabtagene autoleucel, or tisagenlecleucel.

In some embodiments, the methods provided herein can be used as a next in-line treatment for a particular condition or disease after a failed treatment, after a therapeutically ineffective treatment, or after an effective treatment, including in each case following a first-line, second-line, third-line, and additional lines of treatment. In some embodiments, the previous treatment (e.g., the first-line treatment) is a therapeutically ineffective treatment. As used herein, a “therapeutically ineffective” treatment or “failed treatment” or refers to a treatment that produces a less than desired clinical outcome in a patient. For example, with respect to a cancer treatment, a therapeutically ineffective treatment refers to a treatment that does not achieve a desired level of potency, efficacy, and/or specificity. In some embodiments, the failed or therapeutically ineffective prior treatment is characterized by one or more of: (a) a plateau or increase in one or more symptom of the disease, (b) a plateau or a worsening of the extent or state of the disease, (c) a plateau or a worsening of disease progression, (d) an attenuated response to therapy, and (e) disease recurrence. In some embodiments, the disease or disorder is cancer. In some embodiments, the cancer is a lymphoma, leukemia, B-cell acute lymphoblastic leukemia (B-ALL), B-cell Non-Hodgkin lymphoma (B-NHL), or a B-cell chronic lymphoblastic leukemia. In some embodiments, any of the exemplary cancers are also a CD19-negative cancer, a CD22-positive cancer, a CD19-negative/CD22-positive cancer, or a CD19-positive cancer. In certain embodiments, any of the exemplary cancers underwent antigen evasion and no longer express an antigen or have reduced expression of an antigen previously expressed. For example, any of the exemplary cancers can be a CD19-negative and a CD22-positive cancer but were previously CD19-positive and CD22-negative or CD22-positive. In some embodiments, the disease or disorder is a relapsed/refractory CD19-negative cancer, optionally wherein the disease or disorder is a CD19-negative B-ALL relapse characterized by epitope and/or antigen spreading. In some embodiments, the disease or disorder is a cancer that is characterized by rejection, exhaustion, or other failure modes of CD19 CAR-based treatment, including, but not limited to, CD19 mutations, antigen evasion, expression of PDL1, lack of CD58, impaired apoptotic machinery in tumor cell, etc. In some embodiments, the disease or disorder is a cancer that responds poorly to CD19 CAR-based treatment, including, but not limited to, large B-cell lymphoma.

In some embodiments, the prior treatment comprises CD19 specific (CD19) CAR-T cells administered to the patient at a dose of about 50×10⁶ to about 110×10⁶ (e.g., 50×10⁶, 51×10⁶, 52×10⁶, 53×10⁶, 54×10⁶, 55×10⁶, 56×10⁶, 57×10⁶, 58×10⁶, 59×10⁶, 60×10⁶, 61×10⁶, 62×10⁶, 63×10⁶, 64×10⁶, 65×10⁶, 66×10⁶, 67×10⁶, 68×10⁶, 69×10⁶, 70×10⁶, 71×10⁶, 72×10⁶, 73×10⁶, 74×10⁶, 75×10⁶, 76×10⁶, 77×10⁶, 78×10⁶, 79×10⁶, 80×10⁶, 81×10⁶, 82×10⁶, 83×10⁶, 84×10⁶, 85×10⁶, 86×10⁶, 87×10⁶, 88×10⁶, 89×10⁶, 90×10⁶, 91×10⁶, 92×10⁶, 93×10⁶, 94×10⁶, 95×10⁶, 96×10⁶, 97×10⁶, 98×106, 99×10⁶, 100×10⁶, 101×10⁶, 102×10⁶, 103×10⁶, 104×10⁶, 105×10⁶, 106×10⁶, 107×10⁶, 108×10⁶, 109×10⁶, or 110×10⁶) viable CD19 specific CAR-T cells. In some embodiments, the prior treatment comprises viable CD19 specific CAR-T cells that include CD19 specific CAR expressing CD4+ T cells and CD19 specific CAR expressing CD8+ T cells at a ratio of about 1:1. In some embodiments, the prior treatment comprises lisocabtagene maraleucel (BREYANZI®), a structural equivalent thereof, or a functional equivalent thereof.

In some embodiments, a single dose of the prior treatment includes about 50×10⁶ to about 110×10⁶ (e.g., 50×10⁶, 51×10⁶, 52×10⁶, 53×10⁶, 54×10⁶, 55×10⁶, 56×10⁶, 57×10⁶, 58×10⁶, 59×10⁶, 60×10⁶, 61×10⁶, 62×10⁶, 63×10⁶, 64×10⁶, 65×10⁶, 66×10⁶, 67×10⁶, 68×10⁶, 69×10⁶, 70×10⁶, 71×10⁶, 72×10⁶, 73×10⁶, 74×10⁶, 75×10⁶, 76×10⁶, 77×10⁶, 78×10⁶, 79×10⁶, 80×10⁶, 81×10⁶, 82×10⁶, 83×10⁶, 84×10⁶, 85×10⁶, 86×10⁶, 87×10⁶, 88×10⁶, 89×10⁶, 90×10⁶, 91×10⁶, 92×10⁶, 93×10⁶, 94×10⁶, 95×10⁶, 96×10⁶, 97×10⁶, 98×10⁶, 99×10⁶, 100×10⁶, 101×10⁶, 102×10⁶, 103×10⁶, 104×10⁶, 105×10⁶, 106×10⁶, 107×10⁶, 108×10⁶, 109×10⁶, or 110×10⁶) viable CD19 specific CAR-T cells. In some embodiments, the prior treatment comprises viable CD19 specific CAR-T cells that include CD19 specific CAR expressing CD4+ T cells and CD19 specific CAR expressing CD8+ T cells at a ratio of about 1:1. In some embodiments, the prior treatment comprises lisocabtagene maraleucel (BREYANZI®), a structural equivalent thereof, or a functional equivalent thereof.

In some embodiments, the prior treatment comprises CD19 specific (CD19) CAR-T cells administered to the patient at a dose of about 50×10⁶ to about 110×10⁶ (e.g., 50×10⁶, 51×10⁶, 52×10⁶, 53×10⁶, 54×10⁶, 55×10⁶, 56×10⁶, 57×10⁶, 58×10⁶, 59×10⁶, 60×10⁶, 61×10⁶, 62×10⁶, 63×10⁶, 64×10⁶, 65×10⁶, 66×10⁶, 67×10⁶, 68×10⁶, 69×10⁶, 70×10⁶, 71×10⁶, 72×10⁶, 73×10⁶, 74×10⁶, 75×10⁶, 76×10⁶, 77×10⁶, 78×10⁶, 79×10⁶, 80×10⁶, 81×10⁶, 82×10⁶, 83×10⁶, 84×10⁶, 85×10⁶, 86×10⁶, 87×10⁶, 88×10⁶, 89×10⁶, 90×10⁶, 91×10⁶, 92×10⁶, 93×10⁶, 94×10⁶, 95×10⁶, 96×10⁶, 97×10⁶, 98×10⁶, 99×10⁶, 100×10⁶, 101×10⁶, 102×10⁶, 103×10⁶, 104×10⁶, 105×10⁶, 106×10⁶, 107×10⁶, 108×10⁶, 109×10⁶, or 110×10⁶) viable CD19 specific CAR-T cells. In some embodiments, the prior treatment comprises viable CD19 specific CAR-T cells wherein 50% of the viable CD19 specific CAR-T cells are CD19 specific CAR expressing CD4+ T cells and 50% of the viable CD19 specific CAR-T cells are CD19 specific CAR expressing CD8+ T cells. In some embodiments, the prior treatment comprises lisocabtagene maraleucel (BREYANZI®), a structural equivalent thereof, or a functional equivalent thereof.

In some embodiments, the prior treatment comprises CD19 specific (CD19) CAR-T cells administered to the patient at a dose of up to about 2×10⁸ viable CD19 specific CAR-T cells. In some embodiments, the prior treatment comprises CD19 specific (CD19) CAR-T cells administered to the patient at a dose from about 0.2×10⁶ to about 5.0×10⁶ (e.g., about 0.2×10⁶, 0.4×10⁶, 0.5×10⁶, 0.6×10⁶, 0.8×10⁶, 0.9×10⁶, 1.0×10⁶, 1.2×10⁶, 1.4×10⁶, 1.5×10⁶, 1.6×10⁶, 1.8×10⁶, 1.9×10⁶, 2.0×10⁶, 2.2×10⁶, 2.4×10⁶, 2.5×10⁶, 2.6×10⁶, 2.8×10⁶, 2.9×10⁶, 3.0×10⁶, 3.2×10⁶, 3.4×10⁶, 3.5×10⁶, 3.6×10⁶, 3.8×10⁶, 3.9×10⁶, 4.0×10⁶, 4.2×10⁶, 4.4×10⁶, 4.5×10⁶, 4.6×10⁶, 4.8×10⁶, 4.9×10⁶, or 5.0×10⁶) viable CD19 specific CAR-T cells per kg of body weight for a subject with a body weight of about 50 kg or less. In some embodiments, the prior treatment comprises CD19 specific (CD19) CAR-T cells administered to the patient at a dose from about 0.1×10⁸ to about 2.5×10⁸ (e.g., about 0.1×10⁶, 0.2×10⁶, 0.4×10⁶, 0.5×10⁶, 0.6×10⁶, 0.8×10⁶, 0.9×10⁶, 1.0×10⁶, 1.2×10⁶, 1.4×10⁶, 1.5×10⁶, 1.6×10⁶, 1.8×10⁶, 1.9×10⁶, 2.0×10⁶, 2.2×10⁶, 2.4×10⁶, or 2.5×10⁶) viable CD19 specific CAR-T cells for a subject with a body weight of greater than about 50 kg. In some embodiments, the prior treatment comprises CD19 specific (CD19) CAR-T cells administered to the patient at a dose from about 0.6×10⁸ to about 6.0×10⁸ (e.g., about 0.6×10⁸, 0.8×10⁸, 0.9×10⁸, 1.0×10⁸, 1.2×10⁸, 1.4×10⁸, 1.5×10⁸, 1.6×10⁸, 1.8×10⁸, 1.9×10⁸, 2.0×10⁸, 2.2×10⁸, 2.4×10⁸, 2.5×10⁸, 2.6×10⁸, 2.8×10⁸, 2.9×10⁸, 3.0×10⁸, 3.2×10⁸, 3.4×10⁸, 3.5×10⁸, 3.6×10⁸, 3.8×10⁸, 3.9×10⁸, 4.0×10⁸, 4.2×10⁸, 4.4×10⁸, 4.5×10⁸, 4.6×10⁸, 4.8×10⁸, 4.9×10⁸, 5.0×10⁸, 5.2×10⁸, 5.4×10⁸, 5.5×10⁸, 5.6×10⁸, 5.8×10⁸, 5.9×10⁸, or 6.0×10⁸) viable CD19 specific CAR-T cells. In some embodiments, the prior treatment comprises tisagenlecleucel (KYMRIAH®), a structural equivalent thereof, or a functional equivalent thereof.

In some embodiments, a single dose of the prior treatment includes about 0.2×10⁶ to about 5.0×10⁶ (e.g., about 0.2×10⁶, 0.3×10⁶, 0.4×10⁶, 0.5×10⁶, 0.6×10⁶, 0.7×10⁶, 0.8×10⁶, 0.9×10⁶, 1.0×10⁶, 1.1×10⁶, 1.2×10⁶, 1.3×10⁶, 1.4×10⁶, 1.5×10⁶, 1.6×10⁶, 1.7×10⁶, 1.8×10⁶, 1.9×10⁶, 2.0×10⁶, 2.1×10⁶, 2.2×10⁶, 2.3×10⁶, 2.4×10⁶, 2.5×10⁶, 2.6×10⁶, 2.7×10⁶, 2.8×10⁶, 2.9×10⁶, 3.0×10⁶, 3.1×10⁶, 3.2×10⁶, 3.3×10⁶, 3.4×10⁶, 3.5×10⁶, 3.6×10⁶, 3.7×10⁶, 3.8×10⁶, 3.9×10⁶, 4.0×10⁶, 4.1×10⁶, 4.2×10⁶, 4.3×10⁶, 4.4×10⁶, 4.5×10⁶, 4.6×10⁶, 4.7×10⁶, 4.8×10⁶, 4.9×10⁶, or 5.0×10⁶) viable CD19 specific CAR-T cells per kg of body weight for a subject with a body weight of 50 kg or less. In some embodiments, a single dose of the prior treatment includes about 0.1×10⁸ to about 2.5×10⁸ (eq about 0.1×10⁶, 0.2×10⁶, 0.3×10⁶, 0.4×10⁶, 0.5×10⁶, 0.6×10⁶, 0.7×10⁶, 0.8×10⁶, 0.9×10⁶, 1.0×10⁶, 1.1×10⁶, 1.2×10⁶, 1.3×10⁶, 1.4×10⁶, 1.5×10⁶, 1.6×10⁶, 1.7×10⁶, 1.8×10⁶, 1.9×10⁶, 2.0×10⁶, 2.1×10⁶, 2.2×10⁶, 2.3×10⁶, 2.4×10⁶, or 2.5×10⁶) viable CD19 specific CAR-T cells per kg of body weight for a subject with a body weight of more than 50 kg. In some embodiments, a single dose of the prior treatment includes about 0.6×10⁸ to about 6.0×10⁸ (e.g., about 0.6×10⁸, 0.7×10⁸, 0.8×10⁸, 0.9×10⁸, 1.0×10⁸, 1.1×10⁸, 1.2×10⁸, 1.3×10⁸, 1.4×10⁸, 1.5×10⁸, 1.6×10⁸, 1.7×10⁸, 1.8×10⁸, 1.9×10⁸, 2.0×10⁸, 2.1×10⁸, 2.2×10⁸, 2.3×10⁸, 2.4×10⁸, 2.5×10⁸, 2.6×10⁸, 2.7×10⁸, 2.8×10⁸, 2.9×10⁸, 3.0×10⁸, 3.1×10⁸, 3.2×10⁸, 3.3×10⁸, 3.4×10⁸, 3.5×10⁸, 3.6×10⁸, 3.7×10⁸, 3.8×10⁸, 3.9×10⁸, 4.0×10⁸, 4.1×10⁸, 4.2×10⁸, 4.3×10⁸, 4.4×10⁸, 4.5×10⁸, 4.6×10⁸, 4.7×10⁸, 4.8×10⁸, 4.9×10⁸, 5.0×10⁸, 5.1×10⁸, 5.2×10⁸, 5.3×10⁸, 5.4×10⁸, 5.5×10⁸, 5.6×10⁸, 5.7×10⁸, 5.8×10⁸, 5.9×10⁸, or 6.0×10⁸) viable CD19 specific CAR-T cells. In some embodiments, a single infusion bag of the prior treatment includes about 0.6×10⁸ to about 6.0×10⁸ (e.g., about 0.6×10⁸, 0.7×10⁸, 0.8×10⁸, 0.9×10⁸, 1.0×10⁸, 1.1×10⁸, 1.2×10⁸, 1.3×10⁸, 1.4×10⁸, 1.5×10⁸, 1.6×10⁸, 1.7×10⁸, 1.8×10⁸, 1.9×10⁸, 2.0×10⁸, 2.1×10⁸, 2.2×10⁸, 2.3×10⁸, 2.4×10⁸, 2.5×10⁸, 2.6×10⁸, 2.7×10⁸, 2.8×10⁸, 2.9×10⁸, 3.0×10⁸, 3.1×10⁸, 3.2×10⁸, 3.3×10⁸, 3.4×10⁸, 3.5×10⁸, 3.6×10⁸, 3.7×10⁸, 3.8×10⁸, 3.9×10⁸, 4.0×10⁸, 4.1×10⁸, 4.2×10⁸, 4.3×10⁸, 4.4×10⁸, 4.5×10⁸, 4.6×10⁸, 4.7×10⁸, 4.8×10⁸, 4.9×10⁸, 5.0×10⁸, 5.1×10⁸, 5.2×10⁸, 5.3×10⁸, 5.4×10⁸, 5.5×10⁸, 5.6×10⁸, 5.7×10⁸, 5.8×10⁸, 5.9×10⁸, or 6.0×10⁸) viable CD19 specific CAR-T cells in a cell suspension of from about 10 mL to about 50 mL. In some embodiments, the prior treatment comprises tisagenlecleucel (KYMRIAH®), a structural equivalent thereof, or a functional equivalent thereof.

In some embodiments, the prior treatment comprises CD19 specific (CD19) CAR-T cells administered to the patient at a dose of about 2×10⁶ per kg of body weight. In some embodiments, a maximum dose of the prior treatment comprises about 2×10⁸ viable CD19 specific CAR-T cells. In some embodiments, the prior treatment comprises axicabtagene ciloleucel (YESCARTA®), a structural equivalent thereof, or a functional equivalent thereof.

In some embodiments, a single dose of the prior treatment includes about 2×10⁸ viable CD19 specific CAR-T cells. In some embodiments, a single infusion bag of the prior treatment includes about 2×10⁸ viable CD19 specific CAR-T cells in a cell suspension of about 68 mL. In some embodiments, the prior treatment comprises axicabtagene ciloleucel (YESCARTA®), a structural equivalent thereof, or a functional equivalent thereof.

In some embodiments, the prior treatment comprises CD19 specific (CD19) CAR-T cells administered to the patient at a dose of about 2×10⁶ per kg of body weight. In some embodiments, a maximum dose of the prior treatment comprises about 2×10⁸ viable CD19 specific CAR-T cells for a patient of about 100 kg of body weight and above. In some embodiments, the prior treatment comprises brexucabtagene autoleucel (TECARTUS®), a structural equivalent thereof, or a functional equivalent thereof.

In some embodiments, a single dose of the prior treatment includes about 2×10⁸ viable CD19 specific CAR-T cells. In some embodiments, a single infusion bag of the prior treatment includes about 2×10⁸ viable CD19 specific CAR-T cells in a cell suspension of about 68 mL. In some embodiments, the prior treatment comprises brexucabtagene autoleucel (TECARTUS®), a structural equivalent thereof, or a functional equivalent thereof.

### 2. Sensitized Patients

In some embodiments, the engineered CAR-T cells provided herein are useful for the treatment of a patient who has undergone a prior therapy or a previous transplant that caused antigen evasion. In some embodiments, the engineered CAR-T cells provided herein are useful for the treatment of a patient who has undergone a prior therapy or a previous transplant that did not cause antigen evasion. In some embodiments, the prior therapy or previous transplant caused the patient to be sensitized to one or more antigens. In some embodiments, the prior therapy or previous transplant did not cause the patient to be sensitized to one or more antigens.

In some embodiments, the engineered CAR-T cells provided herein are useful for the treatment of a patient sensitized from one or more antigens present in a previous transplant such as, for example, a cell transplant. In certain embodiments, the previous transplant is an allogeneic transplant and the patient is sensitized against one or more alloantigens from the allogeneic transplant. Allogeneic transplants include, but are not limited to, allogeneic cell transplants. In some embodiments, the patient is sensitized patient who is or has been pregnant (e.g., having or having had alloimmunization in pregnancy). In certain embodiments, the patient is sensitized from one or more antigens included in a previous transplant, wherein the previous transplant is a modified human cell. In some embodiments, the modified human cell is a modified autologous human cell. In some embodiments, the previous transplant is a non-human cell. In exemplary embodiments, the previous transplant is a modified non-human cell. In certain embodiments, the previous transplant is a chimera that includes a human component. In certain embodiments, the previous transplant is and/or comprises a CAR-T-cell. In certain embodiments, the previous transplant is and/or comprises a CD19-specific CAR-T-cell. In certain embodiments, the previous transplant is an autologous transplant and the patient is sensitized against one or more autologous antigens from the autologous transplant. In certain embodiments, the previous transplant is an autologous cell. In some embodiments, the sensitized patient has previously received an allogeneic CAR-T cell based therapy or an autologous CAR-T cell based therapy. Non-limiting examples of an autologous CAR-T cell based therapy include brexucabtagene autoleucel (TECARTUS®), axicabtagene ciloleucel (YESCARTA®), idecabtagene vicleucel (ABECMA®), lisocabtagene maraleucel (BREYANZI®), tisagenlecleucel (KYMRIAH®), Descartes-08 and Descartes-11 from Cartesian Therapeutics, CTL110 from Novartis, P-BMCA-101 from Poseida Therapeutics, and AUTO4 from Autolus Limited. Non-limiting examples of an allogeneic CAR-T cell based therapy include UCARTCS from Cellectis, PBCAR19B and PBCAR269A from Precision Biosciences, FT819 from Fate Therapeutics, and CYAD-211 from Clyad Oncology. In some embodiments, after the patient has previously received a first therapy comprising an allogeneic CAR-T cell based therapy or an autologous CAR-T cell based therapy that does not include the cells of the present technology, the sensitized patient is administered a second therapy comprising the cells of the present technology. In some embodiments, after the patient has previously received a first and/or second therapy comprising either an allogeneic CAR-T cell based therapy or an autologous CAR-T cell based therapy that does not include the cells of the present technology, then the sensitized patient is administered a third therapy comprising the cells of the present technology. In some embodiments, after the patient has previously received a series of therapies comprising an allogeneic CAR-T cell based therapy or an autologous CAR-T cell based therapy that does not include the cells of the present technology, then the sensitized patient is administered a subsequent therapy comprising the cells of the present technology. In some embodiments, the methods provided herein is used as next in-line treatment for a particular condition or disease (i) after a failed treatment such as, but not limited to, an allogeneic or autologous CAR-T cell based therapy that does or does not comprise the cells provided herein, (ii) after a therapeutically ineffective treatment such as, but not limited to, an allogeneic or autologous CAR-T cell based therapy that does or does not comprise the cells provided herein, or (iii) after an effective treatment such as, but not limited to, an allogeneic or autologous CAR-T cell based therapy that does or does not comprise the cells provided herein, including in each case in some embodiments following a first-line, second-line, third-line, and additional lines of treatment.

In certain embodiments, the sensitized patient has an allergy and is sensitized to one or more allergens. In exemplary embodiments, the patient has a hay fever, a food allergy, an insect allergy, a drug allergy, and/or atopic dermatitis.

Any suitable method known in the art in view of the present disclosure can be used to determine whether a patient is a sensitized patient. Examples of methods for determining whether a patient is a sensitized patient include, but are not limited to, cell based assays, including complement-dependent cytotoxicity (CDC) and flow cytometry assays, and solid phase assays, including ELISAs and polystyrene bead-based array assays. Other examples of methods for determining whether a patient is a sensitized patient include, but are not limited to, antibody screening methods, percent panel-reactive antibody (PRA) testing, Luminex-based assays, e.g., using single-antigen beads (SABs) and Luminex IgG assays, evaluation of mean fluorescence intensity (MFI) values of HLA antibodies, calculated panel-reactive antibody (cPRA) assays, IgG titer testing, complement-binding assays, IgG subtyping assays, and/or those described in Colvin et al., Circulation. 2019 Mar. 19; 139(12):e553-e578.

### 3. Treatment Properties and Therapeutic Regimens

Therapeutic effectiveness can be measured using any suitable technique known in the art. In some embodiments, the patient produces an immune response to the previous treatment. In some embodiments, the previous treatment is a cell that is rejected by the patient. In some embodiments, the previous treatment included a population of therapeutic cells that include a safety switch that can cause the death of the therapeutic cells, when the safety switch is activated, should they grow and divide in an undesired manner. In some embodiments, the patient produces an immune response as a result of the safety switch induced death of therapeutic cells. In some embodiments, the patient is sensitized from the previous treatment. In exemplary embodiments, the patient is not sensitized by the administered hypoimmunogenic cells.

In some embodiments, the engineered CAR-T cells or progeny thereof have at least one of the following characteristics including, but not limited to: (i) improved persistency and/or durability and/or survival; (ii) increased resistance to native immune cells; (iii) increased cytotoxicity; (iv) improved tumor penetration; (v) enhanced or acquired ADCC; (vi) enhanced ability in migrating, and/or activating or recruiting bystander immune cells, to tumor sites; (vii) enhanced ability to reduce tumor immunosuppression; (viii) improved ability in rescuing tumor antigen escape; and (ix) reduced fratricide (e.g., self-killing), when compared to its native counterpart NK or T cell obtained from peripheral blood, umbilical cord blood, or any other donor tissues, or when compared to a wild-type or control cell or a starting material, or when compared to an autologous CD22 CAR-T therapy.

In some embodiments, the engineered CAR-T cells or progeny thereof exhibit improved persistence and/or durability in the recipient patient. In some embodiments, the engineered CAR-T cells or progeny thereof exhibit improved persistence and/or durability in the recipient patient as compared to, e.g., an autologous CD22 CAR-T therapy. In some embodiments, the engineered CAR-T cells or progeny thereof exhibit at least 40% survival in a patient after 10 days following administration. In various embodiments, the engineered CAR-T cells or progeny thereof exhibit at least 80% survival in a patient after about 2 weeks following administration. In several embodiments, the engineered CAR-T cells or progeny thereof exhibit at least 100% survival in a patient after about 3 weeks following administration. In many embodiments, the engineered CAR-T cells or progeny thereof exhibit at least 150% survival in a patient after about 4 weeks following administration. In some embodiments, the engineered CAR-T cells or progeny thereof persist in the patient for at least 4 weeks, at least 2 months, at least 3 months, at least 4 months, at least 5 months, at least 6 months, at least 7 months, at least 8 months, at least 9 months, at least 10 months, at least 11 months, at least 12 months, or longer.

In some embodiments, the engineered CAR-T cells or progeny thereof exhibit improved efficacy and/or potency and/or elicit a faster therapeutic response in the recipient patient. In some embodiments, the engineered CAR-T cells or progeny thereof exhibit improved efficacy and/or potency and/or elicit a faster therapeutic response in the recipient patient as compared to, e.g., an autologous CD22 CAR-T therapy. In some embodiments, the therapeutic effect of the engineered CAR-T cells or progeny thereof persists for a duration of at least 4 weeks, at least 2 months, at least 3 months, at least 4 months, at least 5 months, at least 6 months, at least 7 months, at least 8 months, at least 9 months, at least 10 months, at least 11 months, at least 12 months, or longer. Therapeutic effectiveness can be measured using any suitable technique known in the art.

The methods of treating a patient are generally through administrations of cells, particularly the engineered CAR-T cells provided herein. As will be appreciated, for all the multiple embodiments described herein related to the cells and/or the timing of therapies, the administering of the cells is accomplished by a method or route that results in at least partial localization of the introduced cells at a desired site. The cells can be implanted directly to the desired site, or alternatively be administered by any appropriate route which results in delivery to a desired location in the subject where at least a portion of the implanted cells or components of the cells remain viable. In some embodiments, the cells are implanted in situ in the desired organ or the desired location of the organ. In some embodiments, the cells are administered to treat a disease or disorder, such as any disease, disorder, condition, and/or symptom thereof that can be alleviated by cell therapy.

In some embodiments, the population of cells is administered at least 1 day, at least 2 days, at least 3 days, at least 4 days, at least 5, days, at least 6 days, at least 1 week, or at least 1 month or more after the patient is sensitized. In some embodiments, the population of cells is administered at least 1 week (e.g., 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, or more) or more after the patient is sensitized or exhibits characteristics or features of sensitization. In some embodiments, the population of cells is administered at least 1 month (e.g., 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, or more) or more after the patient has received the transplant (e.g., an allogeneic transplant), has been pregnant (e.g., having or having had alloimmunization in pregnancy) and/or is sensitized and/or exhibits characteristics and/or features of sensitization.

In some embodiments, the patient who has received a transplant, who has been pregnant (e.g., having or having had alloimmunization in pregnancy), and/or who is sensitized against an antigen (e.g., alloantigens) is administered a dosing regimen comprising a first dose administration of a population of cells described herein, a recovery period after the first dose, and a second dose administration of a population of cells described. In some embodiments, the composite of cell types present in the first population of cells and the second population of cells are different. In certain embodiments, the composite of cell types present in the first population of cells and the second population of cells are the same or substantially equivalent. In many embodiments, the first population of cells and the second population of cells comprises the same cell types. In some embodiments, the first population of cells and the second population of cells comprises different cell types. In some embodiments, the first population of cells and the second population of cells comprises the same percentages of cell types. In other embodiments, the first population of cells and the second population of cells comprises different percentages of cell types.

In some embodiments, the population of cells is administered for the treatment of cancer. In some embodiments, the population of cells is administered for the treatment of cancer and the population of cells is a population of CAR-T cells. In some embodiments, the cancer is selected from the group consisting of lymphoma, leukemia, B cell acute lymphoblastic leukemia (B-ALL), diffuse large B-cell lymphoma, B-cell Non-Hodgkin lymphoma (B-NHL), B-cell chronic lymphoblastic leukemia, liver cancer, pancreatic cancer, breast cancer, ovarian cancer, colorectal cancer, lung cancer, non-small cell lung cancer, acute myeloid lymphoid leukemia, multiple myeloma, gastric cancer, gastric adenocarcinoma, pancreatic adenocarcinoma, glioblastoma, neuroblastoma, lung squamous cell carcinoma, hepatocellular carcinoma, and bladder cancer. In some embodiments, any of the exemplary cancers are also a CD19-negative cancer, a CD22-positive cancer, a CD19-negative/CD22-positive cancer, or a CD19-positive cancer. In certain embodiments, any of the exemplary cancers underwent antigen evasion and no longer express an antigen or have reduced expression of an antigen previously expressed. For example, any of the exemplary cancers can be a CD19-negative and a CD22-positive cancer but were previously CD19-positive and CD22-negative or CD22-positive.

In some embodiments, the recovery period begins following the first administration of the population of hypoimmunogenic cells and ends when such cells are no longer present or detectable in the patient. In some embodiments, the duration of the recovery period is at least 1 week (e.g., 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, or more) or more after the initial administration of the cells. In some embodiments, the duration of the recovery period is at least 1 month (e.g., 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, or more) or more after the initial administration of the cells.

In some embodiments, the administered population of hypoimmunogenic cells elicits a decreased or lower level of systemic TH1 activation in the patient. In some instances, the level of systemic TH1 activation elicited by the cells is at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% lower compared to the level of systemic TH1 activation produced by the administration of immunogenic cells. In some embodiments, the administered population of hypoimmunogenic cells fails to elicit systemic TH1 activation in the patient.

In some embodiments, the administered population of hypoimmunogenic cells elicits a decreased or lower level of immune activation of peripheral blood mononuclear cells (PBMCs) in the patient. In some instances, the level of immune activation of PBMCs elicited by the cells is at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% lower compared to the level of immune activation of PBMCs produced by the administration of immunogenic cells. In some embodiments, the administered population of hypoimmunogenic cells fails to elicit immune activation of PBMCs in the patient.

In some embodiments, the administered population of hypoimmunogenic cells elicits a decreased or lower level of donor-specific IgG antibodies in the patient. In some instances, the level of donor-specific IgG antibodies elicited by the cells is at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% lower compared to the level of donor-specific IgG antibodies produced by the administration of immunogenic cells. In some embodiments, the administered population of hypoimmunogenic cells fails to elicit donor-specific IgG antibodies in the patient.

In some embodiments, the administered population of hypoimmunogenic cells elicits a decreased or lower level of IgM and IgG antibody production in the patient. In some instances, the level of IgM and IgG antibody production elicited by the cells is at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% lower compared to the level of IgM and IgG antibody production produced by the administration of immunogenic cells. In some embodiments, the administered population of hypoimmunogenic cells fails to elicit IgM and IgG antibody production in the patient.

In some embodiments, the administered population of hypoimmunogenic cells elicits a decreased or lower level of cytotoxic T cell killing in the patient. In some instances, the level of cytotoxic T cell killing elicited by the cells is at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% lower compared to the level of cytotoxic T cell killing produced by the administration of immunogenic cells. In some embodiments, the administered population of hypoimmunogenic cells fails to elicit cytotoxic T cell killing in the patient.

As discussed above, provided herein are cells that in certain embodiments can be administered to a patient sensitized against alloantigens such as human leukocyte antigens. In some embodiments, the patient is or has been pregnant, e.g., with alloimmunization in pregnancy (e.g., hemolytic disease of the fetus and newborn (HDFN), neonatal alloimmune neutropenia (NAN) or fetal and neonatal alloimmune thrombocytopenia (FNAIT)). In other words, the patient has or has had a disorder or condition associated with alloimmunization in pregnancy such as, but not limited to, hemolytic disease of the fetus and newborn (HDFN), neonatal alloimmune neutropenia (NAN), and fetal and neonatal alloimmune thrombocytopenia (FNAIT). In some embodiments, the patient has received an allogeneic transplant such as, but not limited to, an allogeneic cell transplant, an allogeneic blood transfusion, an allogeneic tissue transplant, or an allogeneic organ transplant. In some embodiments, the patient exhibits memory B cells against alloantigens. In some embodiments, the patient exhibits memory T cells against alloantigens. Such patients can exhibit both memory B and memory T cells against alloantigens.

Upon administration of the cells described, the patient exhibits no systemic immune response or a reduced level of systemic immune response compared to responses to cells that are not hypoimmunogenic. In some embodiments, the patient exhibits no adaptive immune response or a reduced level of adaptive immune response compared to responses to cells that are not hypoimmunogenic. In some embodiments, the patient exhibits no innate immune response or a reduced level of innate immune response compared to responses to cells that are not hypoimmunogenic. In some embodiments, the patient exhibits no T cell response or a reduced level of T cell response compared to responses to cells that are not hypoimmunogenic. In some embodiments, the patient exhibits no B cell response or a reduced level of B cell response compared to responses to cells that are not hypoimmunogenic.

As is described in further detail herein, provided herein is a population of hypoimmunogenic cells including exogenous CD47 polypeptides, a CD22-specific CAR, and reduced expression of MHC class I human leukocyte antigens; a population of hypoimmunogenic cells including exogenous CD47 polypeptides, a CD22-specific CAR, and reduced expression of MHC class II human leukocyte antigens; and a population of hypoimmunogenic cells including exogenous CD47 polypeptides, a CD22-specific CAR, and reduced expression of MHC class I and class II human leukocyte antigens.

### B. Hypoimmunogenic Cells

In some embodiments, the present disclosure is directed to pluripotent stem cells (e.g., pluripotent stem cells and iPSCs), differentiated cells derived from such pluripotent stem cells (such as, but not limited to, T cells and NK cells), and primary cells (such as, but not limited to, primary T cells and primary NK cells). In some embodiments, the pluripotent stem cells, differentiated cells derived therefrom, such as T cells and NK cells, and primary cells such as primary T cells and primary NK cells, are engineered for reduced expression or lack of expression of MHC class I and/or MHC class II human leukocyte antigens, and in some instances, for reduced expression or lack of expression of a T-cell receptor (TCR) complex. In some embodiments, the hypoimmune (HIP) T cells and primary T cells overexpress CD47 and a CD22-specific chimeric antigen receptor (CAR) in addition to reduced expression or lack of expression of MHC class I and/or MHC class II human leukocyte antigens, and have reduced expression or lack expression of a TCR complex. In some embodiments, the engineered CAR-T cells further comprise one or more additional CARs, wherein the one or more additional CARs comprise an antigen binding domain that binds to any one selected from the group consisting of CD19, CD38, CD123, CD138, BCMA, GPRC5D, CD70, and CD79b. In some embodiments, the one or more additional CARs comprise a CD19-specific CAR. In some instances, the one or more additional CARs comprise a CD38-specific CAR. In some embodiments, the one or more additional CARs comprise a CD123-specific CAR. In some embodiments, the one or more additional CARs comprise a CD138-specific CAR. In some instances, the one or more additional CARs comprise a BCMA-specific CAR. In some instances, the one or more additional CARs comprise a GPRC5D-specific CAR. In some instances, the one or more additional CARs comprise a CD70-specific CAR. In some instances, the one or more additional CARs comprise a CD79b-specific CAR. In some embodiments, the engineered CAR-T cells comprise a bispecific CAR. In some embodiments, the bispecific CAR is a CD19/CD22-bispecific CAR. In some embodiments, the bispecific CAR is a CD19/CD79b-bispecific CAR. In some embodiments, the bispecific CAR is a GPRC5D/CD38-bispecific CAR. In some embodiments, the bispecific CAR is a BCMA/CD38-bispecific CAR. In some embodiments, the cells described express a CD22-specific CAR and a different CAR, such as, but not limited to a CD19-specific CAR, a CD38-specific CAR, a CD123-specific CAR, a CD138-specific CAR, a BCMA-specific CAR, a GPRC5D-specific CAR, a CD70-specific CAR, and a CD79b-specific CAR. In some embodiments, the cells described express a CD123-specific CAR and a different CAR, such as, but not limited to a CD22-specific CAR, a CD38-specific CAR, a CD19-specific CAR, a CD138-specific CAR, and a BCMA-specific CAR. In some embodiments, the cells described express a CD138-specific CAR and a different CAR, such as, but not limited to a CD22-specific CAR, a CD38-specific CAR, a CD123-specific CAR, a CD19-specific CAR, and a BCMA-specific CAR. In some embodiments, the cells described express a BCMA-specific CAR and a different CAR, such as, but not limited to a CD22-specific CAR, a CD38-specific CAR, a CD123-specific CAR, a CD138-specific CAR, and a CD19-specific In some embodiments, the cells are modified or engineered as compared to a wild-type or control cell, including an unaltered or unmodified wild-type cell or control cell. In some embodiments, the wild-type cell or the control cell is a starting material. In some embodiments, the starting material is a primary cell collected from a donor. In some embodiments, the starting material is a primary blood cell collected from a donor, e.g., via a leukopak. In some embodiments, the starting material is otherwise modified or engineered to have altered expression of one or more genes to generate the engineered cell.

In some embodiments, engineered and/or HIP T cells and primary T cells overexpress CD47 and a CD22-specific chimeric antigen receptor (CAR), and include reduced expression of one or more MHC class I and/or class II human leukocyte antigens relative to an unaltered or unmodified wild-type or control cell. In some embodiments, engineered and/or HIP T cells and primary T cells overexpress CD47 and a CD22-specific CAR, and include a genomic modification of the B2M gene. In some embodiments, engineered and/or HIP T cells and primary T cells overexpress CD47 and include a genomic modification of the CIITA gene. In some embodiments, engineered and/or HIP T cells and primary T cells overexpress CD47 and a CD22-specific CAR, and include a genomic modification of the TRAC gene. In some embodiments, engineered and/or HIP T cells and primary T cells overexpress CD47 and a CD22-specific CAR, and include a genomic modification of the TRB gene. In some embodiments, engineered and/or HIP T cells and primary T cells overexpress CD47 and a CD22-specific CAR, and include one or more genomic modifications selected from the group consisting of the B2M, CIITA, TRAC, and TRB genes. In some embodiments, engineered and/or HIP T cells and primary T cells overexpress CD47 and a CD22-specific CAR, and include genomic modifications of the B2M, CIITA, TRAC, and TRB genes. In some embodiments, the cells are B2M^{-/-}, CIITA^{-/-}, TRAC^{-/-}, CD47tg cells that also express a CD22-specific CAR.

In some embodiments, engineered and/or HIP T cells and primary T cells overexpress CD47, a CD22-specific CAR, and a GPRC5D-specific CAR and/or a CD38-specific CAR, and include a genomic modification of the B2M gene. In some embodiments, engineered and/or HIP T cells and primary T cells overexpress CD47 and include a genomic modification of the CIITA gene. In some embodiments, engineered and/or HIP T cells and primary T cells overexpress CD47, a CD22-specific CAR, and a GPRC5D-specific CAR and/or a CD38-specific CAR, and include a genomic modification of the TRAC gene. In some embodiments, engineered and/or HIP T cells and primary T cells overexpress CD47, a CD22-specific CAR, and a GPRC5D-specific CAR and/or a CD38-specific CAR, and include a genomic modification of the TRB gene. In some embodiments, engineered and/or HIP T cells and primary T cells overexpress CD47, a CD22-specific CAR, and a GPRC5D-specific CAR and/or a CD38-specific CAR, and include one or more genomic modifications selected from the group consisting of the B2M, CIITA, TRAC, and TRB genes. In some embodiments, engineered and/or HIP T cells and primary T cells overexpress CD47, a CD22-specific CAR, and a GPRC5D-specific CAR and/or a CD38-specific CAR, and include genomic modifications of the B2M, CIITA, TRAC, and TRB genes. In some embodiments, the cells are B2M^{-/-}, CIITA^{-/-}, TRAC^{-/-}, CD47tg cells that also express a CD22-specific CAR and a GPRC5D-specific CAR and/or a CD38-specific CAR.

In some embodiments, engineered and/or HIP T cells and primary T cells overexpress CD47, a CD22-specific CAR, and a CD70-specific CAR, and include a genomic modification of the B2M gene. In some embodiments, engineered and/or HIP T cells and primary T cells overexpress CD47 and include a genomic modification of the CIITA gene. In some embodiments, engineered and/or HIP T cells and primary T cells overexpress CD47, a CD22-specific CAR, and a CD70-specific CAR, and include a genomic modification of the TRAC gene. In some embodiments, engineered and/or HIP T cells and primary T cells overexpress CD47, a CD22-specific CAR, and a CD70-specific CAR, and include a genomic modification of the TRB gene. In some embodiments, engineered and/or HIP T cells and primary T cells overexpress CD47, a CD22-specific CAR, and a CD70-specific CAR, and include one or more genomic modifications selected from the group consisting of the B2M, CIITA, TRAC, and TRB genes. In some embodiments, engineered and/or HIP T cells and primary T cells overexpress CD47, a CD22-specific CAR, and a CD70-specific CAR, and include genomic modifications of the B2M, CIITA, TRAC, and TRB genes. In some embodiments, the cells are B2M^{-/-}, CIITA^{-/-}, TRAC^{-/-}, CD47tg cells that also express a CD22-specific CAR and a CD70-specific CAR.

In some embodiments, engineered and/or HIP T cells and primary T cells overexpress CD47, a CD22-specific CAR, and a CD70-specific CAR, and include a genomic modification of the B2M gene and of the CD70 gene. In some embodiments, engineered and/or HIP T cells and primary T cells overexpress CD47 and include a genomic modification of the CIITA gene and of the CD70 gene. In some embodiments, engineered and/or HIP T cells and primary T cells overexpress CD47, a CD22-specific CAR, and a CD70-specific CAR, and include a genomic modification of the TRAC gene and of the CD70 gene. In some embodiments, engineered and/or HIP T cells and primary T cells overexpress CD47, a CD22-specific CAR, and a CD70-specific CAR, and include a genomic modification of the TRB gene and of the CD70 gene. In some embodiments, engineered and/or HIP T cells and primary T cells overexpress CD47, a CD22-specific CAR, and a CD70-specific CAR, and include one or more genomic modifications selected from the group consisting of the B2M, CIITA, TRAC, CD70, and TRB genes. In some embodiments, engineered and/or HIP T cells and primary T cells overexpress CD47, a CD22-specific CAR, and a CD70-specific CAR, and include genomic modifications of the B2M, CIITA, TRAC, and TRB genes. In some embodiments, the cells are B2M^{-/-}, CIITA^{-/-}, TRACI^{-/-}, CD70^{-/-}, CD47tg cells that also express a CD22-specific CAR and a CD70-specific CAR.

In some embodiments, engineered and/or HIP T cells and primary T cells overexpress CD47, a CD22-specific CAR, and a CD19/CD79b bi-specific CAR, and include a genomic modification of the B2M gene. In some embodiments, engineered and/or HIP T cells and primary T cells overexpress CD47 and include a genomic modification of the CIITA gene. In some embodiments, engineered and/or HIP T cells and primary T cells overexpress CD47, HLA-E, a CD22-specific CAR, and a CD19/CD79b bi-specific CAR, and include a genomic modification of the TRAC gene. In some embodiments, engineered and/or HIP T cells and primary T cells overexpress CD47, HLA-E, a CD22-specific CAR, and a CD19/CD79b bi-specific CAR, and include a genomic modification of the TRB gene. In some embodiments, engineered and/or HIP T cells and primary T cells overexpress CD47, HLA-E, a CD22-specific CAR, and a CD19/CD79b bi-specific CAR, and include one or more genomic modifications selected from the group consisting of the B2M, CIITA, TRAC, and TRB genes. In some embodiments, engineered and/or HIP T cells and primary T cells overexpress CD47, HLA-E, a CD22-specific CAR, and a CD19/CD79b bi-specific CAR, and include genomic modifications of the B2M, CIITA, TRAC, and TRB genes. In some embodiments, the cells are B2M^{-/-}, CIITA^{-/-}, TRAC^{-/-}, CD47tg cells that also express HLA-E, a CD22-specific CAR and a CD19/CD79b bi-specific CAR.

In some embodiments, engineered and/or HIP T cells and primary T cells overexpress CD47, a CD22-specific CAR, and a BCMA-specific CAR, and include a genomic modification of the B2M gene. In some embodiments, engineered and/or HIP T cells and primary T cells overexpress CD47 and include a genomic modification of the CIITA gene. In some embodiments, engineered and/or HIP T cells and primary T cells overexpress CD47, a CD22-specific CAR, and a BCMA-specific CAR, and include a genomic modification of the TRAC gene. In some embodiments, engineered and/or HIP T cells and primary T cells overexpress CD47, a CD22-specific CAR, and a BCMA-specific CAR, and include a genomic modification of the TRB gene. In some embodiments, engineered and/or HIP T cells and primary T cells overexpress CD47, a CD22-specific CAR, and a BCMA-specific CAR, and include one or more genomic modifications selected from the group consisting of the B2M, CIITA, TRAC, and TRB genes. In some embodiments, engineered and/or HIP T cells and primary T cells overexpress CD47, a CD22-specific CAR, and a BCMA-specific CAR, and include genomic modifications of the B2M, CIITA, TRAC, and TRB genes. In some embodiments, the cells are B2M^{-/-}, CIITA^{-/-}, TRAC^{-/-}, CD47tg cells that also express a CD22-specific CAR and a BCMA-specific CAR.

In some embodiments, engineered and/or HIP T cells and primary T cells overexpress CD47 and a CD22-specific CAR, and include reduced expression of one or more MHC class I and/or class II human leukocyte antigens, and reduced expression of one or more of CD52, CD70, CD155, HLA-A, HLA-B, HLA-C, HLA-DP, HLA-DM, HLA-DOB, HLA-DQ, HLA-DR, RHD, ABO, PCDH11Y, and/or NLGN4Y, relative to an unaltered or unmodified wild-type or control cell. In some embodiments, engineered and/or HIP T cells and primary T cells overexpress CD47 and a CD22-specific CAR, and include a genomic modification of the B2M gene, and reduced expression of one or more of CD52, CD70, CD155, HLA-A, HLA-B, HLA-C, HLA-DP, HLA-DM, HLA-DOB, HLA-DQ, HLA-DR, RHD, ABO, PCDH11Y, and/or NLGN4Y, relative to an unaltered or unmodified wild-type or control cell. In some embodiments, engineered and/or HIP T cells and primary T cells overexpress CD47 and include a genomic modification of the CIITA gene, and reduced expression of one or more of CD52, CD70, CD155, HLA-A, HLA-B, HLA-C, HLA-DP, HLA-DM, HLA-DOB, HLA-DQ, HLA-DR, RHD, ABO, PCDH11Y, and/or NLGN4Y, relative to an unaltered or unmodified wild-type or control cell. In some embodiments, engineered and/or HIP T cells and primary T cells overexpress CD47 and a CD22-specific CAR, and include a genomic modification of the TRAC gene, and reduced expression of one or more of CD52, CD70, CD155, HLA-A, HLA-B, HLA-C, HLA-DP, HLA-DM, HLA-DOB, HLA-DQ, HLA-DR, RHD, ABO, PCDH11Y, and/or NLGN4Y, relative to an unaltered or unmodified wild-type or control cell. In some embodiments, engineered and/or HIP T cells and primary T cells overexpress CD47 and a CD22-specific CAR, and include a genomic modification of the TRB gene, and reduced expression of one or more of CD52, CD70, CD155, HLA-A, HLA-B, HLA-C, HLA-DP, HLA-DM, HLA-DOB, HLA-DQ, HLA-DR, RHD, ABO, PCDH11Y, and/or NLGN4Y, relative to an unaltered or unmodified wild-type or control cell. In some embodiments, engineered and/or HIP T cells and primary T cells overexpress CD47 and a CD22-specific CAR, and include one or more genomic modifications selected from the group consisting of the B2M, CIITA, TRAC, and TRB genes, and reduced expression of one or more of CD52, CD70, CD155, HLA-A, HLA-B, HLA-C, HLA-DP, HLA-DM, HLA-DOB, HLA-DQ, HLA-DR, RHD, ABO, PCDH11Y, and/or NLGN4Y, relative to an unaltered or unmodified wild-type or control cell. In some embodiments, engineered and/or HIP T cells and primary T cells overexpress CD47 and a CD22-specific CAR, and include genomic modifications of the B2M, CIITA, TRAC, and TRB genes, and reduced expression of one or more of CD52, CD70, CD155, HLA-A, HLA-B, HLA-C, HLA-DP, HLA-DM, HLA-DOB, HLA-DQ, HLA-DR, RHD, ABO, PCDH11Y, and/or NLGN4Y, relative to an unaltered or unmodified wild-type or control cell. In some embodiments, the cells are B2M^{-/-}, CIITA^{-/-}, TRAC^{-/-}, CD47tg cells that are also CD52^{-/-}, CD70^{-/-}, CD155^{-/-}, HLA-A^{-/-}, HLA-B^{-/-}, HLA-C^{-/-}, HLA-DP^{-/-}, HLA-DM^{-/-}, HLA-DOB^{-/-}, HLA-DQ^{-/-}, HLA-DR^{-/-}, RHD^{-/-}, ABO^{-/-}, PCDH11Y^{-/-}, and/or NLGN4Y^{-/-}, and that also express a CD22-specific CAR.

In some embodiments, engineered and/or HIP T cells and primary T cells overexpress CD47 and a CD22-specific CAR, and include reduced expression of one or more MHC class I and/or class II human leukocyte antigens, and reduced expression of CD52, relative to an unaltered or unmodified wild-type or control cell. In some embodiments, engineered and/or HIP T cells and primary T cells overexpress CD47 and a CD22-specific CAR, and include a genomic modification of the B2M gene, and reduced expression of CD52, relative to an unaltered or unmodified wild-type or control cell. In some embodiments, engineered and/or HIP T cells and primary T cells overexpress CD47 and include a genomic modification of the CIITA gene, and reduced expression of CD52, relative to an unaltered or unmodified wild-type or control cell. In some embodiments, engineered and/or HIP T cells and primary T cells overexpress CD47 and a CD22-specific CAR, and include a genomic modification of the TRAC gene, and reduced expression of CD52, relative to an unaltered or unmodified wild-type or control cell. In some embodiments, engineered and/or HIP T cells and primary T cells overexpress CD47 and a CD22-specific CAR, and include a genomic modification of the TRB gene, and reduced expression of CD52, relative to an unaltered or unmodified wild-type or control cell. In some embodiments, engineered and/or HIP T cells and primary T cells overexpress CD47 and a CD22-specific CAR, and include one or more genomic modifications selected from the group consisting of the B2M, CIITA, TRAC, and TRB genes, and reduced expression of CD52, relative to an unaltered or unmodified wild-type or control cell. In some embodiments, engineered and/or HIP T cells and primary T cells overexpress CD47 and a CD22-specific CAR, and include genomic modifications of the B2M, CIITA, TRAC, and TRB genes, and reduced expression of CD52, relative to an unaltered or unmodified wild-type or control cell. In some embodiments, the cells are B2M^{-/-}, CIITA^{-/-}, TRAC^{-/-}, CD52^{-/-}, CD47tg cells that also express a CD22-specific CAR.

In some embodiments, engineered and/or HIP T cells and primary T cells overexpress CD47 and a CD22-specific CAR, and include reduced expression of one or more MHC class I and/or class II human leukocyte antigens, and reduced expression of CD70 relative to an unaltered or unmodified wild-type or control cell. In some embodiments, engineered and/or HIP T cells and primary T cells overexpress CD47 and a CD22-specific CAR, and include a genomic modification of the B2M gene, and reduced expression of CD70, relative to an unaltered or unmodified wild-type or control cell. In some embodiments, engineered and/or HIP T cells and primary T cells overexpress CD47 and include a genomic modification of the CIITA gene, and reduced expression of CD70, relative to an unaltered or unmodified wild-type or control cell. In some embodiments, engineered and/or HIP T cells and primary T cells overexpress CD47 and a CD22-specific CAR, and include a genomic modification of the TRAC gene, and reduced expression of CD70, relative to an unaltered or unmodified wild-type or control cell. In some embodiments, engineered and/or HIP T cells and primary T cells overexpress CD47 and a CD22-specific CAR, and include a genomic modification of the TRB gene, and reduced expression of CD70, relative to an unaltered or unmodified wild-type or control cell. In some embodiments, engineered and/or HIP T cells and primary T cells overexpress CD47 and a CD22-specific CAR, and include one or more genomic modifications selected from the group consisting of the B2M, CIITA, TRAC, and TRB genes, and reduced expression of CD70, relative to an unaltered or unmodified wild-type or control cell. In some embodiments, engineered and/or HIP T cells and primary T cells overexpress CD47 and a CD22-specific CAR, and include genomic modifications of the B2M, CIITA, TRAC, and TRB genes, and reduced expression of CD70, relative to an unaltered or unmodified wild-type or control cell. In some embodiments, the cells are B2M^{-/-}, CIITA^{-/-}, TRAC^{-/-}, CD70-1, CD47tg cells that also express a CD22-specific CAR.

In some embodiments, engineered and/or HIP T cells and primary T cells overexpress CD47 and a CD22-specific CAR, and include reduced expression of one or more MHC class I and/or class II human leukocyte antigens, and reduced expression of CD155, relative to an unaltered or unmodified wild-type or control cell. In some embodiments, engineered and/or HIP T cells and primary T cells overexpress CD47 and a CD22-specific CAR, and include a genomic modification of the B2M gene, and reduced expression of CD155, relative to an unaltered or unmodified wild-type or control cell. In some embodiments, engineered and/or HIP T cells and primary T cells overexpress CD47 and include a genomic modification of the CIITA gene, and reduced expression of CD155, relative to an unaltered or unmodified wild-type or control cell. In some embodiments, engineered and/or HIP T cells and primary T cells overexpress CD47 and a CD22-specific CAR, and include a genomic modification of the TRAC gene, and reduced expression of CD155, relative to an unaltered or unmodified wild-type or control cell. In some embodiments, engineered and/or HIP T cells and primary T cells overexpress CD47 and a CD22-specific CAR, and include a genomic modification of the TRB gene, and reduced expression of CD155, relative to an unaltered or unmodified wild-type or control cell. In some embodiments, engineered and/or HIP T cells and primary T cells overexpress CD47 and a CD22-specific CAR, and include one or more genomic modifications selected from the group consisting of the B2M, CIITA, TRAC, and TRB genes, and reduced expression of CD155, relative to an unaltered or unmodified wild-type or control cell. In some embodiments, engineered and/or HIP T cells and primary T cells overexpress CD47 and a CD22-specific CAR, and include genomic modifications of the B2M, CIITA, TRAC, and TRB genes, and reduced expression of CD155, relative to an unaltered or unmodified wild-type or control cell. In some embodiments, the cells are B2M^{-/-}, CIITA^{-/-}, TRAC^{-/-}, CD155^{-/-}, CD47tg cells that also express a CD22-specific CAR.

In some embodiments, engineered and/or HIP T cells are produced by differentiating induced pluripotent stem cells such as engineered and/or hypoimmunogenic induced pluripotent stem cells. In some embodiments, the cells are modified or engineered as compared to a wild-type or control cell, including an unaltered or unmodified wild-type cell or control cell. In some embodiments, the wild-type cell or the control cell is a starting material. In some embodiments, the starting material is a primary cell collected from a donor. In some embodiments, the starting material is a primary blood cell collected from a donor, e.g., via a leukopak. In some embodiments, the starting material is otherwise modified or engineered to have altered expression of one or more genes to generate the engineered cell.

In some embodiments, the engineered and/or HIP T cells and primary T cells are B2M^{-/-}, CIITA^{-/-}, TRB^{-/-}, CD47tg cells that also express CARs. In some embodiments, the cells are B2M^{-/-}, CIITA^{-/-}, TRAC^{-/-}, TRB^{-/-}, CD47tg cells that also express CARs. In certain embodiments, the cells are B2M^{indel/indel}, CIITA^{indel/indel}, TRAC^{indel/indel}, CD47tg cells that also express CARs. In certain embodiments, the cells are B2M^{indel/indel}, CIITA^{indel/indel}, TRB^{indel/indel}, CD47tg cells that also express CARs. In certain embodiments, the cells are B2M^{indel/indel}, CIITA^{indel/indel}, TRAC^{indel/indel}, TRB^{indel/indel}, CD47tg cells that also express CARs. In some embodiments, the engineered or modified cells described are pluripotent stem cells, induced pluripotent stem cells, NK cells differentiated from such pluripotent stem cells and induced pluripotent stem cells, T cells differentiated from such pluripotent stem cells and induced pluripotent stem cells, or primary T cells. Non-limiting examples of primary T cells include CD3+ T cells, CD4+ T cells, CD8+ T cells, naïve T cells, regulatory T (Treg) cells, non-regulatory T cells, Th1 cells, Th2 cells, Th9 cells, Th17 cells, T-follicular helper (Tfh) cells, cytotoxic T lymphocytes (CTL), effector T (Teff) cells, central memory T (Tcm) cells, effector memory T (Tem) cells, effector memory T cells express CD45RA (TEMRA cells), tissue-resident memory (Trm) cells, virtual memory T cells, innate memory T cells, memory stem cell (Tsc), γδ T cells, and any other subtype of T cells. In some embodiments, the primary T cells are selected from a group that includes cytotoxic T-cells, helper T-cells, memory T-cells, regulatory T-cells, tumor infiltrating lymphocytes, and combinations thereof. Non-limiting examples of NK cells and primary NK cells include immature NK cells and mature NK cells. In some embodiments, the cells are modified or engineered as compared to a wild-type or control cell, including an unaltered or unmodified wild-type cell or control cell. In some embodiments, the wild-type cell or the control cell is a starting material. In some embodiments, the starting material is a primary cell collected from a donor. In some embodiments, the starting material is a primary blood cell collected from a donor, e.g., via a leukopak. In some embodiments, the starting material is otherwise modified or engineered to have altered expression of one or more genes to generate the engineered cell.

In some embodiments, the primary T cells are from a pool of primary T cells from one or more donor subjects that are different than the recipient subject (e.g., the patient administered the cells). The primary T cells can be obtained from 1, 2, 3, 4, 5, 6, 7, 8, 9, 10, 20, 50, 100 or more donor subjects and pooled together. The primary T cells can be obtained from 1 or more, 2 or more, 3 or more, 4 or more, 5 or more, 6 or more, 7 or more, 8 or more, 9 or more, 10, or more 20 or more, 50 or more, or 100 or more donor subjects and pooled together. In some embodiments, the primary T cells are harvested from one or a plurality of individuals, and in some instances, the primary T cells or the pool of primary T cells are cultured in vitro. In some embodiments, the primary T cells or the pool of primary T cells are engineered to exogenously express CD47 and cultured in vitro.

In certain embodiments, the primary T cells or the pool of primary T cells are engineered to express a chimeric antigen receptor (CAR). The CAR can be any known to those skilled in the art. Useful CARs include those that bind an antigen selected from a group that includes CD19, CD20, CD22, CD38, CD123, CD138, BCMA, GPRC5D, CD70, and CD79b. In some cases, the CAR is the same or equivalent to those used in FDA-approved CAR-T cell therapies such as, but not limited to, those used in tisagenlecleucel and axicabtagene ciloleucel, or others under investigation in clinical trials.

In some embodiments, the primary T cells or the pool of primary T cells are engineered to exhibit reduced expression of an endogenous T cell receptor compared to unmodified primary T cells. In certain embodiments, the primary T cells or the pool of primary T cells are engineered to exhibit reduced expression of CTLA-4, PD-1, or both CTLA-4 and PD-1, as compared to unmodified primary T cells. Methods of genetically modifying a cell including a T cell are described in detail, for example, in WO2020/018620 and WO2016/183041, the disclosures of which are herein incorporated by reference in their entireties, including the tables, appendices, sequence listing and figures.

In some embodiments, the CAR-T cells comprise a CAR selected from a group including: (a) a first generation CAR comprising an antigen binding domain, a transmembrane domain, and a signaling domain; (b) a second generation CAR comprising an antigen binding domain, a transmembrane domain, and at least two signaling domains; (c) a third generation CAR comprising an antigen binding domain, a transmembrane domain, and at least three signaling domains; and (d) a fourth generation CAR comprising an antigen binding domain, a transmembrane domain, three or four signaling domains, and a domain which upon successful signaling of the CAR induces expression of a cytokine gene.

In some embodiments, the CAR-T cells comprise a CAR comprising an antigen binding domain, a transmembrane, and one or more signaling domains. In some embodiments, the CAR also comprises a linker. In some embodiments, the CAR comprises a CD22 antigen binding domain. In some embodiments, the CAR comprises a CD28 or a CD8a transmembrane domain. In some embodiments, the CAR comprises a CD8a signal peptide. In some embodiments, the CAR comprises a Whitlow linker GSTSGSGKPGSGEGSTKG (SEQ ID NO: 24). In some embodiments, the antigen binding domain of the CAR is selected from a group including, but not limited to, (a) an antigen binding domain targets an antigen characteristic of a neoplastic cell; (b) an antigen binding domain that targets an antigen characteristic of a T cell; (c) an antigen binding domain targets an antigen characteristic of an autoimmune or inflammatory disorder; (d) an antigen binding domain that targets an antigen characteristic of senescent cells; (e) an antigen binding domain that targets an antigen characteristic of an infectious disease; and (f) an antigen binding domain that binds to a cell surface antigen of a cell.

In some embodiments, the CAR further comprises one or more linkers. The format of an scFv is generally two variable domains linked by a flexible peptide sequence, or a “linker,” either in the orientation VH-linker-VL or VL-linker-VH. Any suitable linker known to those in the art in view of the specification can be used in the CARs. Examples of suitable linkers include, but are not limited to, a Whitlow linker GSTSGSGKPGSGEGSTKG (SEQ ID NO: 24), and modifications thereof, an IgG linker, an IgG-based linker, a GS based linker sequence, such as (G₄S)ₙ, wherein n is 1, 2, 3, 4, 5, or more. In some embodiments, the linker is a GS or a gly-ser linker. Exemplary gly-ser polypeptide linkers comprise the amino acid sequence Ser(Gly₄Ser)ₙ, as well as (Gly₄Ser)ₙ and/or (Gly₄Ser₃)ₙ. In some embodiments, n=l. In some embodiments, n=2. In some embodiments, n=3, i.e., Ser(Gly₄Ser)₃. In some embodiments, n=4, i.e., Ser(Gly₄Ser)₄. In some embodiments, n=5. In some embodiments, n=6. In some embodiments, n=7. In some embodiments, n=8. In some embodiments, n=9. In some embodiments, n=10. Another exemplary gly-ser polypeptide linker comprises the amino acid sequence Ser(Gly₄Ser)ₙ. In some embodiments, n=l. In some embodiments, n=2. In some embodiments, n=3. In another embodiment, n=4. In some embodiments, n=5. In some embodiments, n=6. Another exemplary gly-ser polypeptide linker comprises (Gly4Ser)ₙ. In some embodiments, n=l. In some embodiments, n=2. In some embodiments, n=3. In some embodiments, n=4. In some embodiments, n=5. In some embodiments, n=6. Another exemplary gly-ser polypeptide linker comprises (Gly₃Ser)ₙ. In some embodiments, n=l. In some embodiments, n=2. In some embodiments, n=3. In some embodiments, n=4. In another embodiment, n=5. In yet another embodiment, n=6. Another exemplary gly-ser polypeptide linker comprises (Gly₄Ser₃)ₙ. In some embodiments, n=l. In some embodiments, n=2. In some embodiments, n=3. In some embodiments, n=4. In some embodiments, n=5. In some embodiments, n=6. Another exemplary gly-ser polypeptide linker comprises (Gly₃Ser)ₙ. In some embodiments, n=l. In some embodiments, n=2. In some embodiments, n=3. In some embodiments, n=4. In another embodiment, n=5. In yet another embodiment, n=6.

In some embodiments, the antigen binding domain is selected from a group that includes an antibody, an antigen-binding portion or fragment thereof, an scFv, and a Fab. In some embodiments, the antigen binding domain binds to CD19, CD20, CD22, CD38, CD123, CD138, BCMA, GPRC5D, CD70, or CD79b. In some embodiments, the antigen binding domain is an anti-CD19 scFv such as but not limited to FMC63.

In some embodiments, the transmembrane domain comprises one selected from a group that includes a transmembrane region of TCRα, TCRβ, TCRζ, CD3ε, CD3γ, CD3δ, CD3ζ, CD4, CD5, CD8a, CD8P, CD9, CD16, CD28, CD45, CD22, CD33, CD34, CD37, CD40, CD40L/CD154, CD45, CD64, CD80, CD86, OX40/CD134, 4-1BB/CD137, CD154, FcERIγ, VEGFR2, FAS, FGFR2B, and functional variant thereof.

In some embodiments, the signaling domain(s) of the CAR comprises a costimulatory domain(s). For instance, a signaling domain can contain a costimulatory domain or, a signaling domain can contain one or more costimulatory domains. In certain embodiments, the signaling domain comprises a costimulatory domain. In other embodiments, the signaling domains comprise costimulatory domains. In some cases, when the CAR comprises two or more costimulatory domains, two costimulatory domains are not the same. In some embodiments, the costimulatory domains comprise two costimulatory domains that are not the same. In some embodiments, the costimulatory domain enhances cytokine production, CAR-T cell proliferation, and/or CAR-T cell persistence during T cell activation. In some embodiments, the costimulatory domains enhance cytokine production, CAR-T cell proliferation, and/or CAR-T cell persistence during T cell activation.

As described herein, a fourth generation CAR can contain an antigen binding domain, a transmembrane domain, three or four signaling domains, and a domain which upon successful signaling of the CAR induces expression of a cytokine gene. In some instances, the cytokine gene is an endogenous or exogenous cytokine gene of the engineered CAR-T cells. In some cases, the cytokine gene encodes a pro-inflammatory cytokine. In some embodiments, the pro-inflammatory cytokine is selected from a group that includes IL-1, IL-2, IL-9, IL-12, IL-18, TNF, IFN-gamma, and a functional fragment thereof. In some embodiments, the domain which upon successful signaling of the CAR induces expression of the cytokine gene comprises a transcription factor or functional domain or fragment thereof.

In some embodiments, the CAR comprises a CD3 zeta (CD3ζ) domain or an immunoreceptor tyrosine-based activation motif (ITAM), or functional variant thereof. In some embodiments, the CAR comprises (i) a CD3 zeta domain, or an immunoreceptor tyrosine-based activation motif (ITAM), or functional variant thereof; and (ii) a CD28 domain, or a 4-1BB domain, or functional variant thereof. In other embodiments, the CAR comprises (i) a CD3 zeta domain, or an immunoreceptor tyrosine-based activation motif (ITAM), or functional variant thereof; (ii) a CD28 domain or functional variant thereof; and (iii) a 4-1BB domain, or a CD134 domain, or functional variant thereof. In certain embodiments, the CAR comprises (i) a CD3 zeta domain, or an immunoreceptor tyrosine-based activation motif (ITAM), or functional variant thereof; (ii) a CD28 domain or functional variant thereof; (iii) a 4-1BB domain, or a CD134 domain, or functional variant thereof; and (iv) a cytokine or costimulatory ligand transgene. In some embodiments, the CAR comprises a (i) an anti-CD19 scFv; (ii) a CD8a hinge and transmembrane domain or functional variant thereof; (iii) a 4-1BB costimulatory domain or functional variant thereof; and (iv) a CD3ζ signaling domain or functional variant thereof.

Methods for introducing a CAR construct or producing a CAR-T cells are well known to those skilled in the art. Detailed descriptions are found, for example, in Vormittag et al., Curr Opin Biotechnol, 2018, 53, 162-181; and Eyquem et al., Nature, 2017, 543, 113-117.

In some embodiments, the cells derived from primary T cells comprise reduced expression of an endogenous T cell receptor, for example by disruption of an endogenous T cell receptor gene (e.g., T cell receptor alpha constant region (TRAC) or T cell receptor beta constant region (TRB)). In some embodiments, an exogenous nucleic acid encoding a polypeptide as disclosed herein (e.g., a chimeric antigen receptor, CD47, or another tolerogenic factor disclosed herein) is inserted at the disrupted T cell receptor gene. In some embodiments, an exogenous nucleic acid encoding a polypeptide is inserted at a TRAC or a TRB gene locus.

In some embodiments, the cells derived from primary T cells comprise reduced expression of cytotoxic T-lymphocyte-associated protein 4 (CTLA4) and/or programmed cell death (PD1). Methods of reducing or eliminating expression of CTLA4, PD1 and both CTLA4 and PD1 can include any recognized by those skilled in the art, such as but not limited to, genetic modification technologies that utilize rare-cutting endonucleases and RNA silencing or RNA interference technologies. Non-limiting examples of a rare-cutting endonuclease include any Cas protein, TALEN, zinc finger nuclease, meganuclease, and/or homing endonuclease. In some embodiments, an exogenous nucleic acid encoding a polypeptide as disclosed herein (e.g., a chimeric antigen receptor, CD47, or another tolerogenic factor disclosed herein) is inserted at a CTLA4 and/or PD1 gene locus. In some embodiments, the cells are modified or engineered as compared to a wild-type or control cell, including an unaltered or unmodified wild-type cell or control cell. In some embodiments, the wild-type cell or the control cell is a starting material. In some embodiments, the starting material is a primary cell collected from a donor. In some embodiments, the starting material is a primary blood cell collected from a donor, e.g., via a leukopak. In some embodiments, the starting material is otherwise modified or engineered to have altered expression of one or more genes to generate the engineered cell. In some embodiments, the exogenous polynucleotide is inserted into at least one allele of the cell using viral transduction, for example, with a vector. In some embodiments, the vector is a pseudotyped, self-inactivating lentiviral vector that carries the exogenous polynucleotide. In some embodiments, the vector is a self-inactivating lentiviral vector pseudotyped with a vesicular stomatitis VSV-G envelope, and which carries the exogenous polynucleotide. In some embodiments, the exogenous polynucleotide is inserted into at least one allele of the cell using viral transduction. In some embodiments, the exogenous polynucleotide is inserted into at least one allele of the cell using a lentivirus based viral vector.

In some embodiments, a CD47 transgene is inserted into a pre-selected locus of the cell. In some embodiments, a CD47 transgene is inserted into a random locus of the cell. In some embodiments, a transgene encoding a CAR is inserted into a pre-selected locus of the cell. In some embodiments, a transgene encoding a CAR is inserted into a random locus of the cell. In certain embodiments, a CD47 transgene and a transgene encoding a CAR are inserted into a pre-selected locus of the cell. In some embodiments, a transgene encoding a CAR is inserted into a random or pre-selected locus of the cell, including a safe harbor locus, via viral vector transduction/integration. In some embodiments, a CD47 transgene and a transgene encoding a CAR are inserted into a random or pre-selected locus of the cell, including a safe harbor locus, via viral vector transduction/integration. In some embodiments, the vector is a self-inactivating lentiviral vector pseudotyped with a vesicular stomatitis VSV-G envelope. In some embodiments, the transgene encoding a CAR is inserted into at least one allele of the cell using viral transduction. In some embodiments, the exogenous polynucleotide is inserted into at least one allele of the cell using a lentivirus based viral vector. The random and/or pre-selected locus can be a safe harbor or target locus. Non-limiting examples of a safe harbor locus include, but are not limited to, a CCR5 gene locus, a PPP1R12C (also known as AAVS1) gene locus, and a CLYBL gene locus, a Rosa gene locus (e.g., ROSA26 gene locus). Non-limiting examples of a target locus include, but are not limited to, a CXCR4 gene locus, an albumin gene locus, a SHS231 gene locus, an F3 gene locus (also known as CD142), a MICA gene locus, a MICB gene locus, a LRP1 gene locus (also known as a CD91 gene locus), a HMGB1 gene locus, an ABO gene locus, ad RHD gene locus, a FUT1 locus, and a KDM5D gene locus. The CD47 transgene can be inserted in Introns 1 or 2 for PPP1R12C (i.e., AAVS1) or CCR5. The CD47 transgene can be inserted in Exons 1 or 2 or 3 for CCR5. The CD47 transgene can be inserted in intron 2 for CLYBL. The CD47 transgene can be inserted in a 500 bp window in Ch-4:58,976,613 (i.e., SHS231). The CD47 transgene can be insert in any suitable region of the aforementioned safe harbor or target loci that allows for expression of the exogenous polynucleotide, including, for example, an intron, an exon or a coding sequence region in a safe harbor or target locus. In some embodiments, the pre-selected locus is selected from the group consisting of the B2M locus, the CIITA locus, the TRAC locus, and the TRB locus. In some embodiments, the pre-selected locus is the B2M locus. In some embodiments, the pre-selected locus is the CIITA locus. In some embodiments, the pre-selected locus is the TRAC locus. In some embodiments, the pre-selected locus is the TRB locus. In some embodiments, the exogenous polynucleotide is inserted into at least one allele of the cell using viral transduction, for example, with a vector. In some embodiments, the vector is a pseudotyped, self-inactivating lentiviral vector that carries the exogenous polynucleotide. In some embodiments, the vector is a self-inactivating lentiviral vector pseudotyped with a vesicular stomatitis VSV-G envelope, and which carries the exogenous polynucleotide. In some embodiments, the exogenous polynucleotide is inserted into at least one allele of the cell using viral transduction. In some embodiments, the exogenous polynucleotide is inserted into at least one allele of the cell using a lentivirus based viral vector.

In some embodiments, a CD47 transgene and a transgene encoding a CAR are inserted into the same locus. In some embodiments, a CD47 transgene and a transgene encoding a CAR are inserted into different loci. In many instances, a CD47 transgene is inserted into a safe harbor or target locus. In many instances, a transgene encoding a CAR is inserted into a safe harbor or target locus. In some instances, a CD47 transgene is inserted into a B2M locus. In some instances, a transgene encoding a CAR is inserted into a B2M locus. In certain instances, a CD47 transgene is inserted into a CIITA locus. In certain instances, a transgene encoding a CAR is inserted into a CIITA locus. In particular instances, a CD47 transgene is inserted into a TRAC locus. In particular instances, a transgene encoding a CAR is inserted into a TRAC locus. In many other instances, a CD47 transgene is inserted into a TRB locus. In many other instances, a transgene encoding a CAR is inserted into a TRB locus. In some embodiments, a CD47 transgene and a transgene encoding a CAR are inserted into a safe harbor or target locus (e.g., a CCR5 gene locus, a CXCR4 gene locus, a PPP1R12C gene locus, an albumin gene locus, a SHS231 gene locus, a CLYBL gene locus, a Rosa gene locus, an F3 (CD142) gene locus, a MICA gene locus, a MICB gene locus, a LRP1 (CD91) gene locus, a HMGB1 gene locus, an ABO gene locus, an RHD gene locus, a FUT1 locus, and a KDM5D gene locus.

In certain embodiments, a CD47 transgene and a transgene encoding a CAR are inserted into a safe harbor or target locus. In certain embodiments, a CD47 transgene and a transgene encoding a CAR are controlled by a single promoter and are inserted into a safe harbor or target locus. In certain embodiments, a CD47 transgene and a transgene encoding a CAR are controlled by their own promoters and are inserted into a safe harbor or target locus. In certain embodiments, a CD47 transgene and a transgene encoding a CAR are inserted into a TRAC locus. In certain embodiments, a CD47 transgene and a transgene encoding a CAR are controlled by a single promoter and are inserted into a TRAC locus. In certain embodiments, a CD47 transgene and a transgene encoding a CAR are controlled by their own promoters and are inserted into a TRAC locus. In some embodiments, a CD47 transgene and a transgene encoding a CAR are inserted into a TRB locus. In some embodiments, a CD47 transgene and a transgene encoding a CAR are controlled by a single promoter and are inserted into a TRB locus. In some embodiments, a CD47 transgene and a transgene encoding a CAR are controlled by their own promoters and are inserted into a TRB locus. In other embodiments, a CD47 transgene and a transgene encoding a CAR are inserted into a B2M locus. In other embodiments, a CD47 transgene and a transgene encoding a CAR are controlled by a single promoter and are inserted into a B2M locus. In other embodiments, a CD47 transgene and a transgene encoding a CAR are controlled by their own promoters and are inserted into a B2M locus. In various embodiments, a CD47 transgene and a transgene encoding a CAR are inserted into a CIITA locus. In various embodiments, a CD47 transgene and a transgene encoding a CAR are controlled by a single promoter and are inserted into a CIITA locus. In various embodiments, a CD47 transgene and a transgene encoding a CAR are controlled by their own promoters and are inserted into a CIITA locus. In some instances, the promoter controlling expression of any transgene described is a constitutive promoter. In other instances, the promoter for any transgene described is an inducible promoter. In some embodiments, the promoter is an EF1a promoter. In some embodiments, the promoter is CAG promoter. In some embodiments, a CD47 transgene and a transgene encoding a CAR are both controlled by a constitutive promoter. In some embodiments, a CD47 transgene and a transgene encoding a CAR are both controlled by an inducible promoter. In some embodiments, a CD47 transgene is controlled by a constitutive promoter and a transgene encoding a CAR is controlled by an inducible promoter. In some embodiments, a CD47 transgene is controlled by an inducible promoter and a transgene encoding a CAR is controlled by a constitutive promoter. In various embodiments, a CD47 transgene is controlled by an EF1a promoter and a transgene encoding a CAR is controlled by an EF1a promoter. In some embodiments, a CD47 transgene is controlled by a CAG promoter and a transgene encoding a CAR is controlled by a CAG promoter. In some embodiments, a CD47 transgene is controlled by a CAG promoter and a transgene encoding a CAR is controlled by an EF1a promoter. In some embodiments, a CD47 transgene is controlled by an EF1a promoter and a transgene encoding a CAR is controlled by a CAG promoter. In some embodiments, expression of both a CD47 transgene and a transgene encoding a CAR is controlled by a single EF1a promoter. In some embodiments, expression of both a CD47 transgene and a transgene encoding a CAR is controlled by a single CAG promoter.

In another embodiment, the present disclosure disclosed herein is directed to pluripotent stem cells, (e.g., pluripotent stem cells and iPSCs), differentiated cells derived from such pluripotent stem cells (e.g., HIP T cells), and primary T cells that overexpress CD47 (such as exogenously express CD47 proteins), have reduced expression or lack expression of MHC class I and/or MHC class II human leukocyte antigens, and have reduced expression or lack expression of a TCR complex. In some embodiments, the HIP T cells and primary T cells overexpress CD47 (such as exogenously express CD47 proteins), have reduced expression or lack expression of MHC class I and/or MHC class II human leukocyte antigens, and have reduced expression or lack expression of a TCR complex.

In some embodiments, pluripotent stem cells, (e.g., pluripotent stem cells and iPSCs), differentiated cells derived from such pluripotent stem cells (e.g., HIP T cells), and primary T cells overexpress CD47 and include a genomic modification of the B2M gene. In some embodiments, pluripotent stem cells, differentiated cell derived from such pluripotent stem cells and primary T cells overexpress CD47 and include a genomic modification of the CIITA gene. In some embodiments, pluripotent stem cells, T cells differentiated from such pluripotent stem cells and primary T cells overexpress CD47 and include a genomic modification of the TRAC gene. In some embodiments, pluripotent stem cells, T cells differentiated from such pluripotent stem cells and primary T cells overexpress CD47 and include a genomic modification of the TRB gene. In some embodiments, pluripotent stem cells, T cells differentiated from such pluripotent stem cells and primary T cells overexpress CD47 and include one or more genomic modifications selected from the group consisting of the B2M, CIITA, TRAC and TRB genes. In some embodiments, pluripotent stem cells, T cells differentiated from such pluripotent stem cells and primary T cells overexpress CD47 and include genomic modifications of the B2M, CIITA and TRAC genes. In some embodiments, pluripotent stem cells, T cells differentiated from such pluripotent stem cells and primary T cells overexpress CD47 and include genomic modifications of the B2M, CIITA and TRB genes. In some embodiments, pluripotent stem cells, T cells differentiated from such pluripotent stem cells and primary T cells overexpress CD47 and include genomic modifications of the B2M, CIITA, TRAC and TRB genes. In certain embodiments, the pluripotent stem cells, differentiated cell derived from such pluripotent stem cells and primary T cells are B2M^{-/-}, CIITA^{-/-}, TRAC^{-/-}, CD47tg cells. In certain embodiments, the cells are B2M^{-/-}, CIITA^{-/-}, TRB^{-/-}, CD47tg cells. In certain embodiments, the cells are B2M^{-/-}, CIITA^{-/-}, TRAC^{-/-}, TRB−/−, CD47tg cells. In some embodiments, the cells are B2M^{indel/indel}, CIITA^{indel/indel}, TRAC^{indel/indel}, CD47tg cells. In some embodiments, the cells are B2M^{indel/indel}, CIITA^{indel/indel}, TRB^{indel/indel}, CD47tg cells. In some embodiments, the cells are B2M^{indel/indel}, CIITA^{indel/indel}, TRAC^{indel/indel}, TRB^{indel/indel}, CD47tg cells. In some embodiments, the engineered or modified cells described are pluripotent stem cells, T cells differentiated from such pluripotent stem cells or primary T cells. Non-limiting examples of primary T cells include CD3+ T cells, CD4+ T cells, CD8+ T cells, naïve T cells, regulatory T (Treg) cells, non-regulatory T cells, Th1 cells, Th2 cells, Th9 cells, Th17 cells, T-follicular helper (Tfh) cells, cytotoxic T lymphocytes (CTL), effector T (Teff) cells, central memory T (Tcm) cells, effector memory T (Tem) cells, effector memory T cells express CD45RA (TEMRA cells), tissue-resident memory (Trm) cells, virtual memory T cells, innate memory T cells, memory stem cell (Tsc), γδ T cells, and any other subtype of T cells. In some embodiments, the cells are modified or engineered as compared to a wild-type or control cell, including an unaltered or unmodified wild-type cell or control cell. In some embodiments, the wild-type cell or the control cell is a starting material. In some embodiments, the starting material is a primary cell collected from a donor. In some embodiments, the starting material is a primary blood cell collected from a donor, e.g., via a leukopak. In some embodiments, the starting material is otherwise modified or engineered to have altered expression of one or more genes to generate the engineered cell.

In some embodiments, a CD47 transgene is inserted into a pre-selected locus of the cell. The pre-selected locus can be a safe harbor or target locus. Non-limiting examples of a safe harbor or target locus includes a CCR5 gene locus, a CXCR4 gene locus, a PPP1R12C gene locus, an albumin gene locus, a SHS231 gene locus, a CLYBL gene locus, a Rosa gene locus, an F3 (CD142) gene locus, a MICA gene locus, a MICB gene locus, a LRP1 (CD91) gene locus, a HMGB1 gene locus, an ABO gene locus, an RHD gene locus, a FUT1 locus, and a KDM5D gene locus. In some embodiments, the pre-selected locus is the TRAC locus. In some embodiments, a CD47 transgene is inserted into a safe harbor or target locus (e.g., a CCR5 gene locus, a CXCR4 gene locus, a PPP1R12C gene locus, an albumin gene locus, a SHS231 gene locus, a CLYBL gene locus, a Rosa gene locus, an F3 (CD142) gene locus, a MICA gene locus, a MICB gene locus, a LRP1 (CD91) gene locus, a HMGB1 gene locus, an ABO gene locus, an RHD gene locus, a FUT1 locus, and a KDM5D gene locus. In certain embodiments, a CD47 transgene is inserted into the B2M locus. In certain embodiments, a CD47 transgene is inserted into the B2M locus. In certain embodiments, a CD47 transgene is inserted into the TRAC locus. In certain embodiments, a CD47 transgene is inserted into the TRB locus. In some embodiments, the CD47 transgene is inserted into a pre-selected locus of the cell, including a safe harbor locus, via viral vector transduction/integration. In some embodiments, the vector is a self-inactivating lentiviral vector pseudotyped with a vesicular stomatitis VSV-G envelope. In some embodiments, the CD47 transgene is inserted into at least one allele of the cell using viral transduction. In some embodiments, the exogenous polynucleotide is inserted into at least one allele of the cell using a lentivirus based viral vector.

In some instances, expression of a CD47 transgene is controlled by a constitutive promoter. In other instances, expression of a CD47 transgene is controlled by an inducible promoter. In some embodiments, the promoter is an EFlalpha (EF1a) promoter. In some embodiments, the promoter a CAG promoter.

In yet another embodiment, the present disclosure disclosed herein is directed to pluripotent stem cells, (e.g., pluripotent stem cells and iPSCs), T cells derived from such pluripotent stem cells (e.g., HIP T cells), and primary T cells that have reduced expression or lack expression of MHC class I and/or MHC class II human leukocyte antigens and have reduced expression or lack expression of a TCR complex. In some embodiments, the cells have reduced or lack expression of MHC class I antigens, MHC class II antigens, and TCR complexes.

In some embodiments, pluripotent stem cells (e.g., iPSCs), differentiated cells derived from such (e.g., T cells differentiated from such), and primary T cells include a genomic modification of the B2M gene. In some embodiments, pluripotent stem cells (e.g., iPSCs), differentiated cells derived from such (e.g., T cells differentiated from such), and primary T cells include a genomic modification of the CIITA gene. In some embodiments, pluripotent stem cells (e.g., iPSCs), T cells differentiated from such, and primary T cells include a genomic modification of the TRAC gene. In some embodiments, pluripotent stem cells (e.g., iPSCs), T cells differentiated from such, and primary T cells include a genomic modification of the TRB gene. In some embodiments, pluripotent stem cells (e.g., iPSCs), T cells differentiated from such, and primary T cells include one or more genomic modifications selected from the group consisting of the B2M, CIITA and TRAC genes. In some embodiments, pluripotent stem cells (e.g., iPSCs), T cells differentiated from such, and primary T cells include one or more genomic modifications selected from the group consisting of the B2M, CIITA and TRB genes. In some embodiments, pluripotent stem cells (e.g., iPSCs), T cells differentiated from such, and primary T cells include one or more genomic modifications selected from the group consisting of the B2M, CIITA, TRAC and TRB genes. In certain embodiments, the cells including iPSCs, T cells differentiated from such, and primaryT cells are B2M^{-/-}, CIITA^{-/-}, TRAC/cells. In certain embodiments, the cells including iPSCs, T cells differentiated from such, and primary T cells are B2M^{-/-}, CIITA^{-/-}, TRB^{-/-} cells. In some embodiments, the cells including iPSCs, T cells differentiated from such, and primary T cells are B2M^{indel/indel}, CIIITA^{indel/indel}, TRAC^{indel/indel} cells. In some embodiments, the cells including iPSCs, T cells differentiated from such, and primary T cells are B2M^{indel/indel}, CIITA^{indel/indel}, TRB^{indel/indel} cells. In some embodiments, the cells including iPSCs, T cells differentiated from such, and primary T cells are B2M^{indel/indel}, CIITA^{indel/indel}, TRAC^{indel/indel}, TRB^{indel/indel} cells. In some embodiments, the modified cells described are pluripotent stem cells, induced pluripotent stem cells, T cells differentiated from such pluripotent stem cells and induced pluripotent stem cells, or primary T cells. Non-limiting examples of primary T cells include CD3+ T cells, CD4+ T cells, CD8+ T cells, naïve T cells, regulatory T (Treg) cells, non-regulatory T cells, Th1 cells, Th2 cells, Th9 cells, Th17 cells, T-follicular helper (Tfh) cells, cytotoxic T lymphocytes (CTL), effector T (Teff) cells, central memory T (Tcm) cells, effector memory T (Tem) cells, effector memory T cells express CD45RA (TEMRA cells), tissue-resident memory (Trm) cells, virtual memory T cells, innate memory T cells, memory stem cell (Tsc), γδ T cells, and any other subtype of T cells. In some embodiments, the cells are modified or engineered as compared to a wild-type or control cell, including an unaltered or unmodified wild-type cell or control cell. In some embodiments, the wild-type cell or the control cell is a starting material. In some embodiments, the starting material is a primary cell collected from a donor. In some embodiments, the starting material is a primary blood cell collected from a donor, e.g., via a leukopak. In some embodiments, the starting material is otherwise modified or engineered to have altered expression of one or more genes to generate the engineered cell.

Cells of the present disclosure exhibit reduced or lack expression of MHC class I antigens, MHC class II antigens, and/or TCR complexes. Reduction of MHC I and/or MHC II expression can be accomplished, for example, by one or more of the following: (1) targeting the polymorphic HLA alleles (HLA-A, HLA-B, HLA-C) and MHC-II genes directly; (2) removal of B2M, which will prevent surface trafficking of all MHC-I molecules; (3) removal of CIITA, which will prevent surface trafficking of all MHC-II molecules; and/or (4) deletion of components of the MHC enhanceosomes, such as LRC5, RFX5, RFXANK, RFXAP, IRFI, NF-Y (including NFY-A, NFY—B, NFY-C), and CIITA that are critical for HLA expression.

In some embodiments, HLA expression is interfered with by targeting individual HLAs (e.g., knocking out, knocking down, or reducing expression of HLA-A, HLA-B, HLA-C, HLA-DP, HLA-DM, HLA-DOB, HLA-DQ, and/or HLA-DR), targeting transcriptional regulators of HLA expression (e.g., knocking out, knocking down, or reducing expression of NLRC5, CIITA, RFX5, RFXAP, RFXANK, NFY-A, NFY—B, NFY—C and/or IRF-1), blocking surface trafficking of MHC class I molecules (e.g., knocking out, knocking down, or reducing expression of B2M and/or TAP1), and/or targeting with HLA-Razor (see, e.g., WO2016183041).

In some embodiments, the cells disclosed herein including, but not limited to, pluripotent stem cells, induced pluripotent stem cells, differentiated cells derived from such stem cells, and primary T cells do not express one or more human leukocyte antigens (e.g., HLA-A, HLA-B, HLA-C, HLA-DP, HLA-DM, HLA-DOB, HLA-DQ, and/or HLA-DR) corresponding to MHC-I and/or MHC-II and are thus characterized as being hypoimmunogenic. For example, in certain embodiments, the pluripotent stem cells and induced pluripotent stem cells disclosed have been modified such that the stem cell or a differentiated stem cell prepared therefrom do not express or exhibit reduced expression of one or more of the following MHC-I molecules: HLA-A, HLA-B and HLA-C. In some embodiments, one or more of HLA-A, HLA-B and HLA-C may be “knocked-out” of a cell. A cell that has a knocked-out HLA-A gene, HLA-B gene, and/or HLA-C gene may exhibit reduced or eliminated expression of each knocked-out gene.

In some embodiments, guide RNAs, shRNAs, siRNAs, or miRNAs that allow simultaneous deletion of all MHC class I alleles by targeting a conserved region in the HLA genes are identified as HLA Razors. In some embodiments, the gRNAs are part of a CRISPR system. In alternative embodiments, the gRNAs are part of a TALEN system. In some embodiments, an HLA Razor targeting an identified conserved region in HLAs is described in WO2016183041. In some embodiments, multiple HLA Razors targeting identified conserved regions are utilized. It is generally understood that any guide, siRNA, shRNA, or miRNA molecule that targets a conserved region in HLAs can act as an HLA Razor.

Methods provided are useful for inactivation or ablation of MHC class I expression and/or MHC class 11 expression in cells such as but not limited to pluripotent stem cells, differentiated cells, and primary T cells. In some embodiments, genome editing technologies utilizing rare-cutting endonucleases (e.g., the CRISPR/Cas, TALEN, zinc finger nuclease, meganuclease, and homing endonuclease systems) are also used to reduce or eliminate expression of genes involved in an immune response (e.g., by deleting genomic DNA of genes involved in an immune response or by insertions of genomic DNA into such genes, such that gene expression is impacted) in cells. In certain embodiments, genome editing technologies or other gene modulation technologies are used to insert tolerance-inducing factors in human cells, rendering them and the differentiated cells prepared therefrom hypoimmunogenic cells. As such, the engineered CAR-T cells have reduced or eliminated expression of MHC I and MHC II expression. In some embodiments, the cells are nonimmunogenic (e.g., do not induce an innate and/or an adaptive immune response) in a recipient subject.

In some embodiments, the cell includes a modification to increase expression of CD47 and one or more factors selected from the group consisting of DUX4, CD24, CD27, CD35, CD46, CD55, CD59, CD200, HLA-C, HLA-E, HLA-E heavy chain, HLA-G, PD-L1, IDO1, CTLA4-Ig, C1-Inhibitor, IL-10, IL-35, IL-39, FasL, CCL21, CCL22, Mfge8, CD16, CD52, H2-M3, CD16 Fc receptor, IL15-RF, and/or Serpinb9.

In some embodiments, the cell comprises a genomic modification of one or more target polynucleotide sequences that regulate the expression of either MHC class I molecules, MHC class II molecules, or MHC class I and MHC class II molecules. In some embodiments, a genetic editing system is used to modify one or more target polynucleotide sequences. In some embodiments, the targeted polynucleotide sequence is one or more selected from the group including B2M, CIITA, and NLRC5. In some embodiments, the cell comprises a genetic editing modification to the B2M gene. In some embodiments, the cell comprises a genetic editing modification to the CIITA gene. In some embodiments, the cell comprises a genetic editing modification to the NLRC5 gene. In some embodiments, the cell comprises genetic editing modifications to the B2M and CIITA genes. In some embodiments, the cell comprises genetic editing modifications to the B2M and NLRC5 genes. In some embodiments, the cell comprises genetic editing modifications to the CIITA and NLRC5 genes. In numerous embodiments, the cell comprises genetic editing modifications to the B2M, CIITA and NLRC5 genes. In certain embodiments, the genome of the cell has been altered to reduce or delete critical components of HLA expression. In some embodiments, the cells are modified or engineered as compared to a wild-type or control cell, including an unaltered or unmodified wild-type cell or control cell. In some embodiments, the wild-type cell or the control cell is a starting material. In some embodiments, the starting material is a primary cell collected from a donor. In some embodiments, the starting material is a primary blood cell collected from a donor, e.g., via a leukopak. In some embodiments, the starting material is otherwise modified or engineered to have altered expression of one or more genes to generate the engineered cell.

In some embodiments, the present disclosure provides a cell (e.g., stem cell, induced pluripotent stem cell, differentiated cell such as a primary NK cell, CAR-NK cell, primary T cell or CAR-T cell) or population thereof comprising a genome in which a gene has been edited to delete a contiguous stretch of genomic DNA, thereby reducing or eliminating surface expression of MHC class I molecules in the cell or population thereof. In certain embodiments, the present disclosure provides a cell (e.g., stem cell, induced pluripotent stem cell, differentiated cell such as a primary NK cell, CAR-NK cell, primary T cell or CAR-T cell) or population thereof comprising a genome in which a gene has been edited to delete a contiguous stretch of genomic DNA, thereby reducing or eliminating surface expression of MHC class II molecules in the cell or population thereof. In numerous embodiments, the present disclosure provides a cell (e.g., stem cell, induced pluripotent stem cell, differentiated cell, hematopoietic stem cell, primary T cell or CAR-T cell) or population thereof comprising a genome in which one or more genes has been edited to delete a contiguous stretch of genomic DNA, thereby reducing or eliminating surface expression of MHC class I and II molecules in the cell or population thereof.

In certain embodiments, the expression of MHC I molecules and/or MHC II molecules is modulated by targeting and deleting a contiguous stretch of genomic DNA, thereby reducing or eliminating expression of a target gene selected from the group consisting of B2M, CIITA, and NLRC5. In some embodiments, described herein are genetically edited cells (e.g., modified human cells) comprising exogenous CD47 proteins and inactivated or modified CIITA gene sequences, and in some instances, additional gene modifications that inactivate or modify B2M gene sequences. In some embodiments, described herein are genetically edited cells comprising exogenous CD47 proteins and inactivated or modified CIITA gene sequences, and in some instances, additional gene modifications that inactivate or modify NLRC5 gene sequences. In some embodiments, described herein are genetically edited cells comprising exogenous CD47 proteins and inactivated or modified B2M gene sequences, and in some instances, additional gene modifications that inactivate or modify NLRC5 gene sequences. In some embodiments, described herein are genetically edited cells comprising exogenous CD47 proteins and inactivated or modified B2M gene sequences, and in some instances, additional gene modifications that inactivate or modify CIITA gene sequences and NLRC5 gene sequences.

Provided herein are cells exhibiting a modification of one or more targeted polynucleotide sequences that regulates the expression of any one of the following: (a) MHC I antigens, (b) MHC II antigens, (c) TCR complexes, (d) both MHC I and II antigens, and (e) MHC I and II antigens and TCR complexes. In certain embodiments, the modification includes increasing expression of CD47. In some embodiments, the cells include an exogenous or recombinant CD47 polypeptide. In certain embodiments, the modification includes expression of a chimeric antigen receptor. In some embodiments, the cells comprise an exogenous or recombinant chimeric antigen receptor polypeptide.

In some embodiments, the cell includes a genomic modification of one or more targeted polynucleotide sequences that regulates the expression of MHC I antigens, MHC II antigens and/or TCR complexes. In some embodiments, a genetic editing system is used to modify one or more targeted polynucleotide sequences. In some embodiments, the polynucleotide sequence targets one or more genes selected from the group consisting of B2M, CIITA, TRAC, and TRB. In certain embodiments, the genome of a T cell (e.g., a T cell differentiated from hypoimmunogenic iPSCs and a primary T cell) has been altered to reduce or delete critical components of HLA and TCR expression, e.g., HLA-A antigen, HLA-B antigen, HLA-C antigen, HLA-DP antigen, HLA-DQ antigen, HLA-DR antigens, TCR-alpha and TCR-beta.

In some embodiments, the present disclosure provides a cell or population thereof comprising a genome in which a gene has been edited to delete a contiguous stretch of genomic DNA, thereby reducing or eliminating surface expression of MHC class I molecules in the cell or population thereof. In certain embodiments, the present disclosure provides a cell or population thereof comprising a genome in which a gene has been edited to delete a contiguous stretch of genomic DNA, thereby reducing or eliminating surface expression of MHC class II molecules in the cell or population thereof. In certain embodiments, the present disclosure provides a cell or population thereof comprising a genome in which a gene has been edited to delete a contiguous stretch of genomic DNA, thereby reducing or eliminating surface expression of TCR molecules in the cell or population thereof. In numerous embodiments, the present disclosure provides a cell or population thereof comprising a genome in which one or more genes has been edited to delete a contiguous stretch of genomic DNA, thereby reducing or eliminating surface expression of MHC class I and II molecules and TCR complex molecules in the cell or population thereof.

In some embodiments, the cells and methods described herein include genomically editing human cells to cleave CIITA gene sequences as well as editing the genome of such cells to alter one or more additional target polynucleotide sequences such as, but not limited to, B2M TRAC, and TRB. In some embodiments, the cells and methods described herein include genomically editing human cells to cleave B2M gene sequences as well as editing the genome of such cells to alter one or more additional target polynucleotide sequences such as, but not limited to, CIITA, TRAC, and TRB. In some embodiments, the cells and methods described herein include genomically editing human cells to cleave TRAC gene sequences as well as editing the genome of such cells to alter one or more additional target polynucleotide sequences such as, but not limited to, B2M, CIITA, and TRB. In some embodiments, the cells and methods described herein include genomically editing human cells to cleave TRB gene sequences as well as editing the genome of such cells to alter one or more additional target polynucleotide sequences such as, but not limited to, B2M, CIITA, and TRAC.

Provided herein are hypoimmunogenic stem cells comprising reduced expression of HLA-A, HLA-B, HLA-C, HLA-DP, HLA-DM, HLA-DOB, HLA-DQ, HLA-DR, B2M, CIITA, TCR-alpha, and TCR-beta relative to a wild-type stem cell, the hypoimmunogenic stem cell further comprising a set of exogenous polynucleotides comprising a first exogenous polynucleotide encoding CD47 and a second exogenous polynucleotide encoding a chimeric antigen receptor (CAR), wherein the first and/or second exogenous polynucleotides are inserted into a specific locus of at least one allele of the cell. Also provided herein are hypoimmunogenic primary T cells including any subtype of primary T cells comprising reduced expression of HLA-A, HLA-B, HLA-C, HLA-DP, HLA-DM, HLA-DOB, HLA-DQ, HLA-DR, B2M, CIITA, TCR-alpha, and TCR-beta relative to a wild-type primary T cell, the hypoimmunogenic stem cell further comprising a set of exogenous polynucleotides comprising a first exogenous polynucleotide encoding CD47 and a second exogenous polynucleotide encoding a chimeric antigen receptor (CAR), wherein the first and/or second exogenous polynucleotides are inserted into a specific locus of at least one allele of the cell. Further provided herein are hypoimmunogenic T cells differentiated from hypoimmunogenic induced pluripotent stem cells comprising reduced expression of HLA-A, HLA-B, HLA-C, HLA-DP, HLA-DM, HLA-DOB, HLA-DQ, HLA-DR, B2M, CIITA, TCR-alpha, and TCR-beta relative to a wild-type primary T cell, the hypoimmunogenic stem cell further comprising a set of exogenous polynucleotides comprising a first exogenous polynucleotide encoding CD47 and a second exogenous polynucleotide encoding a chimeric antigen receptor (CAR), wherein the first and/or second exogenous polynucleotides are inserted into a specific locus of at least one allele of the cell.

In some embodiments, the population of engineered cells described evades NK cell mediated cytotoxicity upon administration to a recipient patient. In some embodiments, the population of engineered cells evades NK cell mediated cytotoxicity by one or more subpopulations of NK cells. In some embodiments, the population of engineered T cells is protected from cell lysis by NK cells, including immature and/or mature NK cells upon administration to a recipient patient. In some embodiments, the population of engineered cells evades macrophage engulfment upon administration to a recipient patient. In some embodiments, the population of engineered cells does not induce an innate and/or an adaptive immune response to the cell upon administration to a recipient patient.

In some embodiments, the cells described herein comprise a safety switch. The term “safety switch” used herein refers to a system for controlling the expression of a gene or protein of interest that, when downregulated or upregulated, leads to clearance or death of the cell, e.g., through recognition by the host's immune system. A safety switch can be designed to be triggered by an exogenous molecule in case of an adverse clinical event. A safety switch can be engineered by regulating the expression on the DNA, RNA and protein levels. A safety switch includes a protein or molecule that allows for the control of cellular activity in response to an adverse event. In one embodiment, the safety switch is a “kill switch” that is expressed in an inactive state and is fatal to a cell expressing the safety switch upon activation of the switch by a selective, externally provided agent. In one embodiment, the safety switch gene is cis-acting in relation to the gene of interest in a construct. Activation of the safety switch causes the cell to kill solely itself or itself and neighboring cells through apoptosis or necrosis. In some embodiments, the cells described herein, e.g., stem cells, induced pluripotent stem cells, hematopoietic stem cells, primary cells, or differentiated cell, including, but not limited to, T cells, CAR-T cells, NK cells, and/or CAR-NK cells, comprise a safety switch.

In some embodiments, the safety switch comprises a therapeutic agent that inhibits or blocks the interaction of CD47 and SIRPα. In some aspects, the CD47-SIRPa blockade agent is an agent that neutralizes, blocks, antagonizes, or interferes with the cell surface expression of CD47, SIRPα, or both. In some embodiments, the CD47-SIRPα blockade agent inhibits or blocks the interaction of CD47, SIRPα or both. In some embodiments, a CD47-SIRPα blockade agent (e.g., a CD47-SIRPα blocking, inhibiting, reducing, antagonizing, neutralizing, or interfering agent) comprises an agent selected from a group that includes an antibody or fragment thereof that binds CD47, a bispecific antibody that binds CD47, an immunocytokine fusion protein that bind CD47, a CD47 containing fusion protein, an antibody or fragment thereof that binds SIRPα, a bispecific antibody that binds SIRPα, an immunocytokine fusion protein that bind SIRPα, an SIRPα containing fusion protein, and a combination thereof.

In some embodiments, the cells described herein comprise a “suicide gene” (or “suicide switch”). The suicide gene can cause the death of the engineered CAR-T cells should they grow and divide in an undesired manner. The suicide gene ablation approach includes a suicide gene in a gene transfer vector encoding a protein that results in cell killing only when activated by a specific compound. A suicide gene can encode an enzyme that selectively converts a nontoxic compound into highly toxic metabolites. In some embodiments, the cells described herein, e.g., stem cells, induced pluripotent stem cells, hematopoietic stem cells, primary cells, or differentiated cell, including, but not limited to, T cells, CAR-T cells, NK cells, and/or CAR-NK cells, comprise a suicide gene.

In some embodiments, the population of engineered cells described elicits a reduced level of immune activation or no immune activation upon administration to a recipient subject. In some embodiments, the cells elicit a reduced level of systemic TH1 activation or no systemic TH1 activation in a recipient subject. In some embodiments, the cells elicit a reduced level of immune activation of peripheral blood mononuclear cells (PBMCs) or no immune activation of PBMCs in a recipient subject. In some embodiments, the cells elicit a reduced level of donor-specific IgG antibodies or no donor specific IgG antibodies against the cells upon administration to a recipient subject. In some embodiments, the cells elicit a reduced level of IgM and IgG antibody production or no IgM and IgG antibody production against the cells in a recipient subject. In some embodiments, the cells elicit a reduced level of cytotoxic T cell killing of the cells upon administration to a recipient subject.

### 1. Characteristics of Hypolmmunogenic Cells

In some embodiments, the population of hypoimmunogenic stem cells retains pluripotency as compared to a control stem cell (e.g., a wild-type stem cell or immunogenic stem cell). In some embodiments, the population of hypoimmunogenic stem cells retains differentiation potential as compared to a control stem cell (e.g., a wild-type stem cell or immunogenic stem cell).

In some embodiments, the administered population of hypoimmunogenic cells such as hypoimmunogenic CAR-T cells elicits a decreased or lower level of immune activation in the subject or patient. In some instances, the level of immune activation elicited by the cells is at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% lower compared to the level of immune activation produced by the administration of immunogenic cells. In some embodiments, the administered population of hypoimmunogenic cells fails to elicit immune activation in the subject or patient.

In some embodiments, the administered population of hypoimmunogenic cells such as hypoimmunogenic CAR-T cells elicits a decreased or lower level of T cell response in the subject or patient. In some instances, the level of T cell response elicited by the cells is at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% lower compared to the level of T cell response produced by the administration of immunogenic cells. In some embodiments, the administered population of hypoimmunogenic cells fails to elicit a T cell response to the cells in the subject or patient.

In some embodiments, the administered population of hypoimmunogenic cells such as hypoimmunogenic CAR-T cells elicits a decreased or lower level of NK cell response in the subject or patient. In some instances, the level of NK cell response elicited by the cells is at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% lower compared to the level of NK cell response produced by the administration of immunogenic cells. In some embodiments, the administered population of hypoimmunogenic cells fails to elicit an NK cell response to the cells in the subject or patient.

In some embodiments, the administered population of hypoimmunogenic cells such as hypoimmunogenic CAR-T cells elicits a decreased or lower level of macrophage engulfment in the subject or patient. In some instances, the level of NK cell response elicited by the cells is at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% lower compared to the level of macrophage engulfment produced by the administration of immunogenic cells. In some embodiments, the administered population of hypoimmunogenic cells fails to elicit macrophage engulfment of the cells in the subject or patient.

In some embodiments, the administered population of hypoimmunogenic cells such as hypoimmunogenic CAR-T cells elicits a decreased or lower level of systemic TH1 activation in the subject or patient. In some instances, the level of systemic TH1 activation elicited by the cells is at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% lower compared to the level of systemic TH1 activation produced by the administration of immunogenic cells. In some embodiments, the administered population of hypoimmunogenic cells fails to elicit systemic TH1 activation in the subject or patient.

In some embodiments, the administered population of hypoimmunogenic cells such as hypoimmunogenic CAR-T cells elicits a decreased or lower level of NK cell killing in the subject or patient. In some instances, the level of NK cell killing elicited by the cells is at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% lower compared to the level of NK cell killing produced by the administration of immunogenic cells. In some embodiments, the administered population of hypoimmunogenic cells fails to elicit NK cell killing in the subject or patient.

In some embodiments, the administered population of hypoimmunogenic cells such as hypoimmunogenic CAR-T cells elicits a decreased or lower level of immune activation of peripheral blood mononuclear cells (PBMCs) in the subject or patient. In some instances, the level of immune activation of PBMCs elicited by the cells is at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% lower compared to the level of immune activation of PBMCs produced by the administration of immunogenic cells. In some embodiments, the administered population of hypoimmunogenic cells fails to elicit immune activation of PBMCs in the subject or patient.

In some embodiments, the administered population of hypoimmunogenic cells such as hypoimmunogenic CAR-T cells elicits a decreased or lower level of donor-specific IgG antibodies in the subject or patient. In some instances, the level of donor-specific IgG antibodies elicited by the cells is at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% lower compared to the level of donor-specific IgG antibodies produced by the administration of immunogenic cells. In some embodiments, the administered population of hypoimmunogenic cells fails to elicit donor-specific IgG antibodies in the subject or patient.

In some embodiments, the administered population of hypoimmunogenic cells such as hypoimmunogenic CAR-T cells elicits a decreased or lower level of donor-specific IgM antibodies in the subject or patient. In some instances, the level of donor-specific IgM antibodies elicited by the cells is at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% lower compared to the level of donor-specific IgM antibodies produced by the administration of immunogenic cells. In some embodiments, the administered population of hypoimmunogenic cells fails to elicit donor-specific IgM antibodies in the subject or patient.

In some embodiments, the administered population of hypoimmunogenic cells such as hypoimmunogenic CAR-T cells elicits a decreased or lower level of IgM and IgG antibody production in the subject or patient. In some instances, the level of IgM and IgG antibody production elicited by the cells is at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% lower compared to the level of IgM and IgG antibody production produced by the administration of immunogenic cells. In some embodiments, the administered population of hypoimmunogenic cells fails to elicit IgM and IgG antibody production in the subject or patient.

In some embodiments, the administered population of hypoimmunogenic cells such as hypoimmunogenic CAR-T cells elicits a decreased or lower level of cytotoxic T cell killing in the subject or patient. In some instances, the level of cytotoxic T cell killing elicited by the cells is at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% lower compared to the level of cytotoxic T cell killing produced by the administration of immunogenic cells. In some embodiments, the administered population of hypoimmunogenic cells fails to elicit cytotoxic T cell killing in the subject or patient.

In some embodiments, the administered population of hypoimmunogenic cells such as hypoimmunogenic CAR-T cells elicits a decreased or lower level of complement-dependent cytotoxicity (CDC) in the subject or patient. In some instances, the level of CDC elicited by the cells is at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% lower compared to the level of CDC produced by the administration of immunogenic cells. In some embodiments, the administered population of hypoimmunogenic cells fails to elicit CDC in the subject or patient.

In some embodiments, an engineered cell described herein comprises one or more nucleotide sequences encoding one or more safety switches. In some embodiments, an engineered cell described herein comprises a transgene encoding two or more tolerogenic factors. In certain of these embodiments, a nucleotide sequence encoding the safety switch is in the form of a polycistronic construct connected by one or more cleavage sites. In some embodiments, a nucleotide sequence encoding the safety switch is in the form of a polycistronic construct with a nucleotide sequence encoding one or more tolerogenic factors. In some embodiments, in 5′ to 3′ order, a coding sequence for the safety switch can precede a coding sequence for the tolerogenic factor or vice versa. In some embodiments, one or more cleavage sites comprise a self-cleaving site, for example, a 2A site. In some embodiments, a 2A site comprises a T2A, P2A, E2A, or F2A site. In some embodiments, one or more cleavage sites further comprise a protease site, for example, a furin site. In some embodiments, a furin site comprises an FC1, FC2, or FC3 site. In some embodiments, a protease site precedes a 2A site in the 5′ to 3′ order.

In some embodiments, a nucleotide sequence encoding the safety switch is in the same expression cassette comprising the transgene encoding one or more tolerogenic factors. In some embodiments, a nucleotide sequence encoding a safety switch is in a different expression cassette from an expression cassette comprising a transgene encoding one or more tolerogenic factors. In some embodiments wherein a tolerogenic factor is CD47, any of the agents that can inhibit or block the interaction of CD47 and SIRPα can be used in any combination to serve as safety switches for any of the engineered immune evasive cells disclosed herein.

In some embodiments, a safety switch is or comprises a herpes simplex virus thymidine kinase (HSVtk), cytosine deaminase (CyD), nitroreductase (NTR), purine nucleoside phosphorylase (PNP), horseradish peroxidase, inducible caspase 9 (iCasp9), rapamycin-activated caspase (rapaCasp) such as rapaCasp 9, CCR4, CD16, CD19, CD20, CD30, EGFR, GD2, HER1, HER2, MUC1, PSMA, or RQR8.

C. Select Molecules with Expression That May be Modulated

### 1. CIITA

In some embodiments, the technologies disclosed herein modulate (e.g., reduces or eliminates) the expression of MHC II genes by targeting and modulating (e.g., reducing or eliminating) Class II transactivator (CIITA) expression. In some embodiments, the modulation occurs using a CRISPR/Cas system. CIITA is a member of the LR or nucleotide binding domain (NBD) leucine-rich repeat (LRR) family of proteins and regulates the transcription of MHC II by associating with the MHC enhanceosome.

In some embodiments, the target polynucleotide sequence of the present disclosure is a variant of CIITA. In some embodiments, the target polynucleotide sequence is a homolog of CIITA. In some embodiments, the target polynucleotide sequence is an ortholog of CIITA.

In some embodiments, reduced or eliminated expression of CIITA reduces or eliminates expression of one or more of the following: HLA-DP, HLA-DM, HLA-DOA, HLA-DOB, HLA-DQ, and HLA-DR.

In some embodiments, the cells described herein comprise gene modifications at the gene locus encoding the CIITA protein. In other words, the cells comprise a genetic modification at the CIITA locus. In some instances, the nucleotide sequence encoding the CIITA protein is set forth in RefSeq. No. NM_000246.4 and NCBI Genbank No. U18259. In some instances, the CIITA gene locus is described in NCBI Gene ID No. 4261. In certain cases, the amino acid sequence of CIITA is depicted as NCBI GenBank No. AAA88861.1. Additional descriptions of the CIITA protein and gene locus can be found in Uniprot No. P33076, HGNC Ref. No. 7067, and OMIM Ref. No. 600005.

In some embodiments, the engineered CAR-T cells outlined herein comprise a genetic modification targeting the CIITA gene. In some embodiments, the genetic modification targeting the CIITA gene by the rare-cutting endonuclease comprises a Cas protein or a polynucleotide encoding a Cas protein, and at least one guide ribonucleic acid sequence for specifically targeting the CIITA gene. In some embodiments, the at least one guide ribonucleic acid sequence for specifically targeting the CIITA gene is selected from the group consisting of SEQ ID NOS:5184-36352 of Table 12 of WO2016183041, which is herein incorporated by reference. In some embodiments, the cell has a reduced ability to induce an innate and/or an adaptive immune response in a recipient subject. In some embodiments, an exogenous nucleic acid encoding a polypeptide as disclosed herein (e.g., a chimeric antigen receptor, CD47, or another tolerogenic factor disclosed herein) is inserted at the CIITA gene.

Assays to test whether the CIITA gene has been inactivated are known and described herein. In some embodiments, the resulting genetic modification of the CIITA gene by PCR and the reduction of HLA-II expression can be assays by FACS analysis. In another embodiment, CIITA protein expression is detected using a Western blot of cells lysates probed with antibodies to the CIITA protein. In another embodiment, reverse transcriptase polymerase chain reactions (RT-PCR) are used to confirm the presence of the inactivating genetic modification. In some embodiments, the exogenous polynucleotide is inserted into at least one allele of the cell using viral transduction, for example, with a vector. In some embodiments, the vector is a pseudotyped, self-inactivating lentiviral vector that carries the exogenous polynucleotide. In some embodiments, the vector is a self-inactivating lentiviral vector pseudotyped with a vesicular stomatitis VSV-G envelope, and which carries the exogenous polynucleotide. In some embodiments, the exogenous polynucleotide is inserted into at least one allele of the cell using viral transduction. In some embodiments, the exogenous polynucleotide is inserted into at least one allele of the cell using a lentivirus based viral vector.

### 2. B2M

In some embodiments, the technologies disclosed herein modulate (e.g., reduce or eliminate) the expression of MHC-I genes by targeting and modulating (e.g., reducing or eliminating) expression of the accessory chain B2M. In some embodiments, the modulation occurs using a CRISPR/Cas system. By modulating (e.g., reducing or deleting) expression of B2M, surface trafficking of MHC-I molecules is blocked and the cell rendered hypoimmunogenic. In some embodiments, the cell has a reduced ability to induce an innate and/or an adaptive immune response in a recipient subject.

In some embodiments, the target polynucleotide sequence of the present disclosure is a variant of B2M. In some embodiments, the target polynucleotide sequence is a homolog of B2M. In some embodiments, the target polynucleotide sequence is an ortholog of B2M.

In some embodiments, decreased or eliminated expression of B2M reduces or eliminates expression of one or more of the following MHC I molecules: HLA-A, HLA-B, and HLA-C.

In some embodiments, the cells described herein comprise gene modifications at the gene locus encoding the B2M protein. In other words, the cells comprise a genetic modification at the B2M locus. In some instances, the nucleotide sequence encoding the B2M protein is set forth in RefSeq. No. NM_004048.4 and Genbank No. AB021288.1. In some instances, the B2M gene locus is described in NCBI Gene ID No. 567. In certain cases, the amino acid sequence of B2M is depicted as NCBI GenBank No. BAA35182.1. Additional descriptions of the B2M protein and gene locus can be found in Uniprot No. P61769, HGNC Ref. No. 914, and OMIM Ref. No. 109700.

In some embodiments, the engineered CAR-T cells outlined herein comprise a genetic modification targeting the B2M gene. In some embodiments, the genetic modification targeting the B2M gene by the rare-cutting endonuclease comprises a Cas protein or a polynucleotide encoding a Cas protein, and at least one guide ribonucleic acid sequence for specifically targeting the B2M gene. In some embodiments, the at least one guide ribonucleic acid sequence for specifically targeting the B2M gene is selected from the group consisting of SEQ ID NOS:81240-85644 of Table 15 of WO2016183041, which is herein incorporated by reference. In some embodiments, an exogenous nucleic acid encoding a polypeptide as disclosed herein (e.g., a chimeric antigen receptor, CD47, or another tolerogenic factor disclosed herein) is inserted at the B2M gene. In some embodiments, the exogenous polynucleotide is inserted into at least one allele of the cell using viral transduction, for example, with a vector. In some embodiments, the vector is a pseudotyped, self-inactivating lentiviral vector that carries the exogenous polynucleotide. In some embodiments, the vector is a self-inactivating lentiviral vector pseudotyped with a vesicular stomatitis VSV-G envelope, and which carries the exogenous polynucleotide. In some embodiments, the exogenous polynucleotide is inserted into at least one allele of the cell using viral transduction. In some embodiments, the exogenous polynucleotide is inserted into at least one allele of the cell using a lentivirus based viral vector.

Assays to test whether the B2M gene has been inactivated are known and described herein. In some embodiments, the resulting genetic modification of the B2M gene by PCR and the reduction of HLA-I expression can be assays by FACS analysis. In another embodiment, B2M protein expression is detected using a Western blot of cells lysates probed with antibodies to the B2M protein. In another embodiment, RT-PCR are used to confirm the presence of the inactivating genetic modification.

### 3. NLRC5

In many embodiments, the technologies disclosed herein modulate (e.g., reduce or eliminate) the expression of MHC-I genes by targeting and modulating (e.g., reducing or eliminating) expression of the NLR family, CARD domain containing 5/NOD27/CLR16.1 (NLRC5). In some embodiments, the modulation occurs using a CRISPR/Cas system. NLRC5 is a critical regulator of MHC-1-mediated immune responses and, similar to CIITA, NLRC5 is highly inducible by IFN-γ and can translocate into the nucleus. NLRC5 activates the promoters of MHC-I genes and induces the transcription of MHC-I as well as related genes involved in MHC-I antigen presentation.

In some embodiments, the target polynucleotide sequence is a variant of NLRC5. In some embodiments, the target polynucleotide sequence is a homolog of NLRC5. In some embodiments, the target polynucleotide sequence is an ortholog of NLRC5.

In some embodiments, decreased or eliminated expression of NLRC5 reduces or eliminates expression of one or more of the following MHC I molecules—HLA-A, HLA-B, and HLA-C.

In some embodiments, the cells outlined herein comprise a genetic modification targeting the NLRC5 gene. In some embodiments, the genetic modification targeting the NLRC5 gene by the rare-cutting endonuclease comprises a Cas protein or a polynucleotide encoding a Cas protein, and at least one guide ribonucleic acid sequence for specifically targeting the NLRC5 gene. In some embodiments, the at least one guide ribonucleic acid sequence for specifically targeting the NLRC5 gene is selected from the group consisting of SEQ ID NOS:36353-81239 of Appendix 3 or Table 14 of WO2016183041, the disclosure is incorporated by reference in its entirety.

Assays to test whether the NLRC5 gene has been inactivated are known and described herein. In some embodiments, the resulting genetic modification of the NLRC5 gene by PCR and the reduction of HLA-I expression can be assays by FACS analysis. In another embodiment, NLRC5 protein expression is detected using a Western blot of cells lysates probed with antibodies to the NLRC5 protein. In another embodiment, RT-PCR are used to confirm the presence of the inactivating genetic modification.

### 4. TRAC

In many embodiments, the technologies disclosed herein modulate (e.g., reduce or eliminate) the expression of TCR genes including the TRAC gene by targeting and modulating (e.g., reducing or eliminating) expression of the constant region of the T cell receptor alpha chain. In some embodiments, the modulation occurs using a CRISPR/Cas system. By modulating (e.g., reducing or deleting) expression of TRAC, surface trafficking of TCR molecules is blocked. In some embodiments, the cell also has a reduced ability to induce an innate and/or an adaptive immune response in a recipient subject.

In some embodiments, the target polynucleotide sequence of the present disclosure is a variant of TRAC. In some embodiments, the target polynucleotide sequence is a homolog of TRAC. In some embodiments, the target polynucleotide sequence is an ortholog of TRAC.

In some embodiments, decreased or eliminated expression of TRAC reduces or eliminates TCR surface expression.

In some embodiments, the cells, such as, but not limited to, pluripotent stem cells, induced pluripotent stem cells, T cells differentiated from induced pluripotent stem cells, primary T cells, and cells derived from primary T cells comprise gene modifications at the gene locus encoding the TRAC protein. In other words, the cells comprise a genetic modification at the TRAC locus. In some instances, the nucleotide sequence encoding the TRAC protein is set forth in Genbank No. X02592.1. In some instances, the TRAC gene locus is described in RefSeq. No. NG_001332.3 and NCBI Gene ID No. 28755. In certain cases, the amino acid sequence of TRAC is depicted as Uniprot No. P01848. Additional descriptions of the TRAC protein and gene locus can be found in Uniprot No. P01848, HGNC Ref. No. 12029, and OMIM Ref. No. 186880.

In some embodiments, the engineered CAR-T cells outlined herein comprise a genetic modification targeting the TRAC gene. In some embodiments, the genetic modification targeting the TRAC gene by the rare-cutting endonuclease comprises a Cas protein or a polynucleotide encoding a Cas protein, and at least one guide ribonucleic acid sequence for specifically targeting the TRAC gene. In some embodiments, the at least one guide ribonucleic acid sequence for specifically targeting the TRAC gene is selected from the group consisting of SEQ ID NOS:532-609 and 9102-9797 of US20160348073, which is herein incorporated by reference.

Assays to test whether the TRAC gene has been inactivated are known and described herein. In some embodiments, the resulting genetic modification of the TRAC gene by PCR and the reduction of TCR expression can be assays by FACS analysis. In another embodiment, TRAC protein expression is detected using a Western blot of cells lysates probed with antibodies to the TRAC protein. In another embodiment, RT-PCR are used to confirm the presence of the inactivating genetic modification.

### 5. TRB

In many embodiments, the technologies disclosed herein modulate (e.g., reduce or eliminate) the expression of TCR genes including the gene encoding T cell antigen receptor, beta chain (e.g., the TRB, TRBC, or TCRB gene) by targeting and modulating (e.g., reducing or eliminating) expression of the constant region of the T cell receptor beta chain. In some embodiments, the modulation occurs using a CRISPR/Cas system. By modulating (e.g., reducing or deleting) expression of TRB, surface trafficking of TCR molecules is blocked. In some embodiments, the cell also has a reduced ability to induce an innate and/or an adaptive immune response in a recipient subject.

In some embodiments, the target polynucleotide sequence of the present disclosure is a variant of TRB. In some embodiments, the target polynucleotide sequence is a homolog of TRB. In some embodiments, the target polynucleotide sequence is an ortholog of TRB.

In some embodiments, decreased or eliminated expression of TRB reduces or eliminates TCR surface expression.

In some embodiments, the cells, such as, but not limited to, pluripotent stem cells, induced pluripotent stem cells, T cells differentiated from induced pluripotent stem cells, primary T cells, and cells derived from primary T cells comprise gene modifications at the gene locus encoding the TRB protein. In other words, the cells comprise a genetic modification at the TRB gene locus. In some instances, the nucleotide sequence encoding the TRB protein is set forth in UniProt No. PODSE2. In some instances, the TRB gene locus is described in RefSeq. No. NG_001333.2 and NCBI Gene ID No. 6957. In certain cases, the amino acid sequence of TRB is depicted as Uniprot No. P01848. Additional descriptions of the TRB protein and gene locus can be found in GenBank No. L36092.2, Uniprot No. PODSE2, and HGNC Ref. No. 12155.

In some embodiments, the engineered CAR-T cells outlined herein comprise a genetic modification targeting the TRB gene. In some embodiments, the genetic modification targeting the TRB gene by the rare-cutting endonuclease comprises a Cas protein or a polynucleotide encoding a Cas protein, and at least one guide ribonucleic acid sequence for specifically targeting the TRB gene. In some embodiments, the at least one guide ribonucleic acid sequence for specifically targeting the TRB gene is selected from the group consisting of SEQ ID NOS:610-765 and 9798-10532 of US20160348073, which is herein incorporated by reference.

Assays to test whether the TRB gene has been inactivated are known and described herein. In some embodiments, the resulting genetic modification of the TRB gene by PCR and the reduction of TCR expression can be assays by FACS analysis. In another embodiment, TRB protein expression is detected using a Western blot of cells lysates probed with antibodies to the TRB protein. In another embodiment, RT-PCR are used to confirm the presence of the inactivating genetic modification.

### 6. CD142

In many embodiments, the technologies disclosed herein modulate (e.g., reduce or eliminate) the expression of CD142, which is also known as tissue factor, factor Ill, and F3. In some embodiments, the modulation occurs using a gene editing system (e.g., CRISPR/Cas).

In some embodiments, the target polynucleotide sequence is CD142 or a variant of CD142. In some embodiments, the target polynucleotide sequence is a homolog of CD142. In some embodiments, the target polynucleotide sequence is an ortholog of CD142.

In some embodiments, the cells outlined herein comprise a genetic modification targeting the CD142 gene. In some embodiments, the genetic modification targeting the CD142 gene by the rare-cutting endonuclease comprises a Cas protein or a polynucleotide encoding a Cas protein, and at least one guide ribonucleic acid (gRNA) sequence for specifically targeting the CD142 gene. Useful methods for identifying gRNA sequences to target CD142 are described below.

Assays to test whether the CD142 gene has been inactivated are known and described herein. In some embodiments, the resulting genetic modification of the CD142 gene by PCR and the reduction of CD142 expression can be assays by FACS analysis. In another embodiment, CD142 protein expression is detected using a Western blot of cells lysates probed with antibodies to the CD142 protein. In another embodiment, RT-PCR are used to confirm the presence of the inactivating genetic modification.

Useful genomic, polynucleotide and polypeptide information about the human CD142 are provided in, for example, the GeneCard Identifier GC01M094530, HGNC No. 3541, NCBI Gene ID 2152, NCBI RefSeq Nos. NM_001178096.1, NM_001993.4, NP_001171567.1, and NP_001984.1, UniProt No. P13726, and the like.

### 7. CD52

In many embodiments, the technologies disclosed herein modulate (e.g., reduce or eliminate) the expression of CD52, which is also known as CAMPATH-1 antigen, CDw52, Cambridge pathology 1 antigen, Epididymal secretory protein E5, Human epididymis-specific protein 5, He5, and CDW52. In some embodiments, the modulation occurs using a gene editing system (e.g., CRISPR/Cas).

In some embodiments, the target polynucleotide sequence is CD52 or a variant of CD52. In some embodiments, the target polynucleotide sequence is a homolog of CD52. In some embodiments, the target polynucleotide sequence is an ortholog of CD52.

In some embodiments, the cells outlined herein comprise a genetic modification targeting the CD52 gene. In some embodiments, the genetic modification targeting the CD52 gene by the rare-cutting endonuclease comprises a Cas protein or a polynucleotide encoding a Cas protein, and at least one guide ribonucleic acid (gRNA) sequence for specifically targeting the CD52 gene.

Assays to test whether the CD52 gene has been inactivated are known and described herein. In some embodiments, the resulting genetic modification of the CD52 gene by PCR and the reduction of CD52 expression can be assays by FACS analysis. In another embodiment, CD52 protein expression is detected using a Western blot of cells lysates probed with antibodies to the CD52 protein. In another embodiment, RT-PCR are used to confirm the presence of the inactivating genetic modification.

Useful genomic, polynucleotide and polypeptide information about the human CD52 are provided in, for example, the GeneCard Identifier CD52, HGNC No. 1804, NCBI Gene ID 1043, NCBI RefSeq Nos. NP_001794.2 and NM_001803.2, UniProt No. P31358, and the like.

### 8. CD70

In many embodiments, the technologies disclosed herein modulate (e.g., reduce or eliminate) the expression of CD70, which is also known as CD70 antigen, CD27 ligand, CD27-L, Tumor necrosis factor ligand superfamily member 7, CD27L, CD27LG, and TNFSF7. In some embodiments, the modulation occurs using a gene editing system (e.g., CRISPR/Cas).

In some embodiments, the target polynucleotide sequence is CD70 or a variant of CD70. In some embodiments, the target polynucleotide sequence is a homolog of CD70. In some embodiments, the target polynucleotide sequence is an ortholog of CD70.

In some embodiments, the cells outlined herein comprise a genetic modification targeting the CD70 gene. In some embodiments, the genetic modification targeting the CD70 gene by the rare-cutting endonuclease comprises a Cas protein or a polynucleotide encoding a Cas protein, and at least one guide ribonucleic acid (gRNA) sequence for specifically targeting the CD70 gene.

Assays to test whether the CD70 gene has been inactivated are known and described herein. In some embodiments, the resulting genetic modification of the CD70 gene by PCR and the reduction of CD70 expression can be assays by FACS analysis. In another embodiment, CD70 protein expression is detected using a Western blot of cells lysates probed with antibodies to the CD70 protein. In another embodiment, RT-PCR are used to confirm the presence of the inactivating genetic modification.

Useful genomic, polynucleotide and polypeptide information about the human CD70 are provided in, for example, the GeneCard Identifier CD70, HGNC No. 11937, NCBI Gene ID 970, NCBI RefSeq Nos. NP_001243.1, NM_001252.4, NP_001317261.1, and NM_001330332.1, UniProt No. P32970, and the like. 9. CD155

In many embodiments, the technologies disclosed herein modulate (e.g., reduce or eliminate) the expression of CD155, which is also known as Poliovirus receptor, Nectin-like protein 5, NECL-5, PVR, and PVS. In some embodiments, the modulation occurs using a gene editing system (e.g., CRISPR/Cas).

In some embodiments, the target polynucleotide sequence is CD155 or a variant of CD155. In some embodiments, the target polynucleotide sequence is a homolog of CD155. In some embodiments, the target polynucleotide sequence is an ortholog of CD155.

In some embodiments, the cells outlined herein comprise a genetic modification targeting the CD155 gene. In some embodiments, the genetic modification targeting the CD155 gene by the rare-cutting endonuclease comprises a Cas protein or a polynucleotide encoding a Cas protein, and at least one guide ribonucleic acid (gRNA) sequence for specifically targeting the CD155 gene.

Assays to test whether the CD155 gene has been inactivated are known and described herein. In some embodiments, the resulting genetic modification of the CD155 gene by PCR and the reduction of CD155 expression can be assays by FACS analysis. In another embodiment, CD155 protein expression is detected using a Western blot of cells lysates probed with antibodies to the CD155 protein. In another embodiment, RT-PCR are used to confirm the presence of the inactivating genetic modification.

Useful genomic, polynucleotide and polypeptide information about the human CD155 are provided in, for example, the GeneCard Identifier PVR, HGNC No. 9705, NCBI Gene ID 5817, NCBI RefSeq Nos. NP_001129240.1, NM_001135768.2, NP_001129241.1, NM_001135769.2, NP_001129242.2, NM_001135770.3, NP_006496.4, and NM_006505.4, UniProt No. P15151, and the like. 10. CTLA-4

In some embodiments, the target polynucleotide sequence is CTLA-4 or a variant of CTLA-4. In some embodiments, the target polynucleotide sequence is a homolog of CTLA-4. In some embodiments, the target polynucleotide sequence is an ortholog of CTLA-4.

In some embodiments, the cells outlined herein comprise a genetic modification targeting the CTLA-4 gene. In certain embodiments, primary T cells comprise a genetic modification targeting the CTLA-4 gene. The genetic modification can reduce expression of CTLA-4 polynucleotides and CTLA-4 polypeptides in T cells includes primary T cells and CAR-T cells. In some embodiments, the genetic modification targeting the CTLA-4 gene by the rare-cutting endonuclease comprises a Cas protein or a polynucleotide encoding a Cas protein, and at least one guide ribonucleic acid (gRNA) sequence for specifically targeting the CTLA-4 gene. Useful methods for identifying gRNA sequences to target CTLA-4 are described below.

Assays to test whether the CTLA-4 gene has been inactivated are known and described herein. In some embodiments, the resulting genetic modification of the CTLA-4 gene by PCR and the reduction of CTLA-4 expression can be assays by FACS analysis. In another embodiment, CTLA-4 protein expression is detected using a Western blot of cells lysates probed with antibodies to the CTLA-4 protein. In another embodiment, RT-PCR are used to confirm the presence of the inactivating genetic modification.

Useful genomic, polynucleotide and polypeptide information about the human CTLA-4 are provided in, for example, the GeneCard Identifier GC02P203867, HGNC No. 2505, NCBI Gene ID 1493, NCBI RefSeq Nos. NM_005214.4, NM_001037631.2, NP_001032720.1 and NP_005205.2, UniProt No. P16410, and the like.

### 11. PD-1

In some embodiments, the target polynucleotide sequence is PD-1 or a variant of PD-1. In some embodiments, the target polynucleotide sequence is a homolog of PD-1. In some embodiments, the target polynucleotide sequence is an ortholog of PD-1.

In some embodiments, the cells outlined herein comprise a genetic modification targeting the gene encoding the programmed cell death protein 1 (PD-1) protein or the PDCD1 gene. In certain embodiments, primary T cells comprise a genetic modification targeting the PDCD1 gene. The genetic modification can reduce expression of PD-1 polynucleotides and PD-1 polypeptides in T cells includes primary T cells and CAR-T cells. In some embodiments, the genetic modification targeting the PDCD1 gene by the rare-cutting endonuclease comprises a Cas protein or a polynucleotide encoding a Cas protein, and at least one guide ribonucleic acid (gRNA) sequence for specifically targeting the PDCD1 gene. Useful methods for identifying gRNA sequences to target PD-1 are described below.

Assays to test whether the PDCD1 gene has been inactivated are known and described herein. In some embodiments, the resulting genetic modification of the PDCD1 gene by PCR and the reduction of PD-1 expression can be assays by FACS analysis. In another embodiment, PD-1 protein expression is detected using a Western blot of cells lysates probed with antibodies to the PD-1 protein. In another embodiment, RT-PCR are used to confirm the presence of the inactivating genetic modification.

Useful genomic, polynucleotide and polypeptide information about human PD-1 including the PDCD1 gene are provided in, for example, the GeneCard Identifier GC02M241849, HGNC No. 8760, NCBI Gene ID 5133, Uniprot No. Q15116, and NCBI RefSeq Nos. NM_005018.2 and NP_005009.2.

### 12. CD47

In some embodiments, the present disclosure provides a cell or population thereof that has been modified to express the tolerogenic factor (e.g., immunomodulatory polypeptide) CD47. In some embodiments, the present disclosure provides a method for altering a cell genome to express CD47. In some embodiments, the stem cell expresses exogenous CD47. In some instances, the cell expresses an expression vector comprising a nucleotide sequence encoding a human CD47 polypeptide. In some embodiments, the cell is genetically modified to comprise an integrated exogenous polynucleotide encoding CD47 using homology-directed repair. In some instances, the cell expresses a nucleotide sequence encoding a human CD47 polypeptide such that the nucleotide sequence is inserted into at least one allele of a safe harbor or target locus. In some instances, the cell expresses a nucleotide sequence encoding a human CD47 polypeptide wherein the nucleotide sequence is inserted into at least one allele of an AAVS1 locus. In some instances, the cell expresses a nucleotide sequence encoding a human CD47 polypeptide wherein the nucleotide sequence is inserted into at least one allele of an CCR5 locus. In some instances, the cell expresses a nucleotide sequence encoding a human CD47 polypeptide wherein the nucleotide sequence is inserted into at least one allele of a safe harbor or target gene locus, such as, but not limited to, a CCR5 gene locus, a CXCR4 gene locus, a PPP1R12C gene locus, an albumin gene locus, a SHS231 gene locus, a CLYBL gene locus, a Rosa gene locus, an F3 (CD142) gene locus, a MICA gene locus, a MICB gene locus, a LRP1 (CD91) gene locus, a HMGB1 gene locus, an ABO gene locus, an RHD gene locus, a FUT1 locus, and a KDM5D gene locus. In some instances, the cell expresses a nucleotide sequence encoding a human CD47 polypeptide wherein the nucleotide sequence is inserted into at least one allele of a TRAC locus.

CD47 is a leukocyte surface antigen and has a role in cell adhesion and modulation of integrins. It is expressed on the surface of a cell and signals to circulating macrophages not to eat the cell.

In some embodiments, the cell outlined herein comprises a nucleotide sequence encoding a CD47 polypeptide has at least 95% sequence identity (e.g., 95%, 96%, 97%, 98%, 99%, or more) to an amino acid sequence as set forth in NCBI Ref. Sequence Nos. NP_001768.1 and NP_942088.1. In some embodiments, the cell outlined herein comprises a nucleotide sequence encoding a CD47 polypeptide having an amino acid sequence as set forth in NCBI Ref. Sequence Nos. NP_001768.1 and NP_942088.1. In some embodiments, the cell comprises a nucleotide sequence for CD47 having at least 85% sequence identity (e.g., 85%, 86%, 87%, 88%, 89%, 90%, 91%, 92%, 93%, 94%, 95%, 96%, 97%, 98%, 99%, or more) to the sequence set forth in NCBI Ref. Nos. NM_001777.3 and NM_198793.2. In some embodiments, the cell comprises a nucleotide sequence for CD47 as set forth in NCBI Ref. Sequence Nos. NM_001777.3 and NM_198793.2. In some embodiments, the nucleotide sequence encoding a CD47 polynucleotide is a codon optimized sequence. In some embodiments, the nucleotide sequence encoding a CD47 polynucleotide is a human codon optimized sequence.

In some embodiments, the cell comprises a CD47 polypeptide having at least 95% sequence identity (e.g., 95%, 96%, 97%, 98%, 99%, or more) to an amino acid sequence as set forth in NCBI Ref. Sequence Nos. NP_001768.1 and NP_942088.1. In some embodiments, the cell outlined herein comprises a CD47 polypeptide having an amino acid sequence as set forth in NCBI Ref. Sequence Nos. NP_001768.1 and NP_942088.1.

Exemplary amino acid sequences of human CD47 with a signal sequence and without a signal sequence are provided in Table 1.

TABLE 1
Amino acid sequences of human CD47

| | SEQ | | |
|---|---|---|---|
| | ID | | Amino acid |
| Protein | NO: | Sequence | residues |
| Human CD47 | 136 | QLLFNKTKSVEFTFCNDTVVIPCFVTNMEAQNTTEVYVKWKFKGRDI | aa 19-323 |
| (without | | YTFDGALNKSTVPTDFSSAKIEVSQLLKGDASLKMDKSDAVSHTGNYT | |
| signal | | CEVTELTREGETIIELKYRVVSWFSPNENILIVIFPIFAILLFWGQFGIKTL | |
| sequence) | | KYRSGGMDEKTIALLVAGLVITVIVIVGAILFVPGEYSLKNATGLGLIVT | |
| | | STGILILLHYYVFSTAIGLTSFVIAILVIQVIAYILAVVGLSLCIAACIPMHG | |
| | | PLLISGLSILALAQLLGLVYMKFVASNQKTIQPPRKAVEEPLNAFKESKG | |
| | | MMNDE | |
| Human CD47 | 137 | MWPLVAALLLGSACCGSAQLLFNKTKSVEFTFCNDTVVIPCFVTNME | aa 1-323 |
| (with signal | | AQNTTEVYVKWKFKGRDIYTFDGALNKSTVPTDFSSAKIEVSQLLKGD | |
| sequence) | | ASLKMDKSDAVSHTGNYTCEVTELTREGETIIELKYRVVSWFSPNENIL | |
| | | IVIFPIFAILLFWGQFGIKTLKYRSGGMDEKTIALLVAGLVITVIVIVGAIL | |
| | | FVPGEYSLKNATGLGLIVTSTGILILLHYYVFSTAIGLTSFVIAILVIQVIAYI | |
| | | LAVVGLSLCIAACIPMHGPLLISGLSILALAQLLGLVYMKFVASNQKTIQ | |
| | | PPRKAVEEPLNAFKESKGMMNDE | |

In some embodiments, the cell comprises a CD47 polypeptide having at least 95% sequence identity (e.g., 95%, 96%, 97%, 98%, 99%, or more) to the amino acid sequence of SEQ ID NO:136. In some embodiments, the cell comprises a CD47 polypeptide having the amino acid sequence of SEQ ID NO:136. In some embodiments, the cell comprises a CD47 polypeptide having at least 95% sequence identity (e.g., 95%, 96%, 97%, 98%, 99%, or more) to the amino acid sequence of SEQ ID NO:137. In some embodiments, the cell comprises a CD47 polypeptide having the amino acid sequence of SEQ ID NO:137.

In some embodiments, the cell comprises a nucleotide sequence encoding a CD47 polypeptide having at least 95% sequence identity (e.g., 95%, 96%, 97%, 98%, 99%, or more) to the amino acid sequence of SEQ ID NO:136. In some embodiments, the cell comprises a nucleotide sequence encoding a CD47 polypeptide having the amino acid sequence of SEQ ID NO:136. In some embodiments, the cell comprises a nucleotide sequence encoding a CD47 polypeptide having at least 95% sequence identity (e.g., 95%, 96%, 97%, 98%, 99%, or more) to the amino acid sequence of SEQ ID NO:137. In some embodiments, the cell comprises a nucleotide sequence encoding a CD47 polypeptide having the amino acid sequence of SEQ ID NO:137. In some embodiments, the nucleotide sequence is codon optimized for expression in a particular cell.

In some embodiments, a suitable gene editing system (e.g., CRISPR/Cas system or any of the gene editing systems described herein) is used to facilitate the insertion of a polynucleotide encoding CD47, into a genomic locus of the hypoimmunogenic cell. In some cases, the polynucleotide encoding CD47 is inserted into a safe harbor or target locus, such as but not limited to, an AAVS1, CCR5, CLYBL, ROSA26, SHS231, F3 (CD142), MICA, MICB, LRP1 (CD91), HMGB1, ABO, RHD, FUT1, or KDM5D gene locus. In some embodiments, the polynucleotide encoding CD47 is inserted into a B2M gene locus, a CIITA gene locus, a TRAC gene locus, or a TRB gene locus. In some embodiments, the polynucleotide encoding CD47 is inserted into any one of the gene loci depicted in Table 33 or 36 provided herein. In certain embodiments, the polynucleotide encoding CD47 is operably linked to a promoter.

In some embodiments, the polynucleotide encoding CD47 is inserted into at least one allele of the T cell using viral transduction. In some embodiments, the polynucleotide encoding CD47 is inserted into at least one allele of the T cell using a lentivirus based viral vector. In some embodiments, the lentivirus based viral vector is a pseudotyped, self-inactivating lentiviral vector that carries the polynucleotide encoding CD47. In some embodiments, the lentivirus based viral vector is a self-inactivating lentiviral vector pseudotyped with a vesicular stomatitis VSV-G envelope, and which carries the polynucleotide encoding CD47.

In another embodiment, CD47 protein expression is detected using a Western blot of cell lysates probed with antibodies against the CD47 protein. In another embodiment, RT-PCR are used to confirm the presence of the exogenous CD47 mRNA.

### 13. CD24

In some embodiments, the present disclosure provides a cell or population thereof that has been modified to express the tolerogenic factor (e.g., immunomodulatory polypeptide) CD24. In some embodiments, the present disclosure provides a method for altering a cell genome to express CD24. In some embodiments, the stem cell expresses exogenous CD24. In some instances, the cell expresses an expression vector comprising a nucleotide sequence encoding a human CD24 polypeptide. In some embodiments, the exogenous polynucleotide is inserted into at least one allele of the cell using viral transduction, for example, with a vector. In some embodiments, the vector is a pseudotyped, self-inactivating lentiviral vector that carries the exogenous polynucleotide. In some embodiments, the vector is a self-inactivating lentiviral vector pseudotyped with a vesicular stomatitis VSV-G envelope, and which carries the exogenous polynucleotide. In some embodiments, the exogenous polynucleotide is inserted into at least one allele of the cell using viral transduction. In some embodiments, the exogenous polynucleotide is inserted into at least one allele of the cell using a lentivirus based viral vector.

CD24 which is also referred to as a heat stable antigen or small-cell lung cancer cluster 4 antigen is a glycosylated glycosylphosphatidylinositol-anchored surface protein (Pirruccello et al., J Immunol, 1986, 136, 3779-3784; Chen et al., Glycobiology, 2017, 57, 800-806). It binds to Siglec-10 on innate immune cells. Recently it has been shown that CD24 via Siglec-10 acts as an innate immune checkpoint (Barkal et al., Nature, 2019, 572, 392-396).

In some embodiments, the cell outlined herein comprises a nucleotide sequence encoding a CD24 polypeptide has at least 95% sequence identity (e.g., 95%, 96%, 97%, 98%, 99%, or more) to an amino acid sequence set forth in NCBI Ref. Nos. NP_001278666.1, NP_001278667.1, NP_001278668.1, and NP_037362.1. In some embodiments, the cell outlined herein comprises a nucleotide sequence encoding a CD24 polypeptide having an amino acid sequence set forth in NCBI Ref. Nos. NP_001278666.1, NP_001278667.1, NP_001278668.1, and NP_037362.1.

In some embodiments, the cell comprises a nucleotide sequence having at least 85% sequence identity (e.g., 85%, 86%, 87%, 88%, 89%, 90%, 91%, 92%, 93%, 94%, 95%, 96%, 97%, 98%, 99%, or more) to the sequence set forth in NCBI Ref. Nos. NM_00129737.1, NM_00129738.1, NM_001291739.1, and NM_013230.3. In some embodiments, the cell comprises a nucleotide sequence as set forth in NCBI Ref. Nos. NM_00129737.1, NM_00129738.1, NM_001291739.1, and NM_013230.3.

In some embodiments, a suitable gene editing system (e.g., CRISPR/Cas system or any of the gene editing systems described herein) is used to facilitate the insertion of a polynucleotide encoding CD24, into a genomic locus of the hypoimmunogenic cell. In some cases, the polynucleotide encoding CD24 is inserted into a safe harbor or target locus, such as but not limited to, an AAVS1, CCR5, CLYBL, ROSA26, SHS231, F3 (CD142), MICA, MICB, LRP1 (CD91), HMGB1, ABO, RHD, FUT1, or KDM5D gene locus. In some embodiments, the polynucleotide encoding CD24 is inserted into a B2M gene locus, a CIITA gene locus, a TRAC gene locus, or a TRB gene locus. In some embodiments, the polynucleotide encoding CD24 is inserted into any one of the gene loci depicted in Table 33 or 36 provided herein. In certain embodiments, the polynucleotide encoding CD24 is operably linked to a promoter.

In another embodiment, CD24 protein expression is detected using a Western blot of cells lysates probed with antibodies against the CD24 protein. In another embodiment, RT-PCR are used to confirm the presence of the exogenous CD24 mRNA.

In some embodiments, a suitable gene editing system (e.g., CRISPR/Cas system or any of the gene editing systems described herein) is used to facilitate the insertion of a polynucleotide encoding CD24, into a genomic locus of the hypoimmunogenic cell. In some cases, the polynucleotide encoding CD24 is inserted into a safe harbor or target locus, such as but not limited to, an AAVS1, CCR5, CLYBL, ROSA26, SHS231, F3 (also known as CD142), MICA, MICB, LRP1 (also known as CD91), HMGB1, ABO, RHD, FUT1, or KDM5D gene locus. In some embodiments, the polynucleotide encoding CD24 is inserted into a B2M gene locus, a CIITA gene locus, a TRAC gene locus, or a TRB gene locus. In some embodiments, the polynucleotide encoding CD24 is inserted into any one of the gene loci depicted in Table 33 or 36 provided herein. In certain embodiments, the polynucleotide encoding CD24 is operably linked to a promoter.

### 14. DUX4

In some embodiments, the present disclosure provides a cell (e.g., stem cell, induced pluripotent stem cell, differentiated cell, hematopoietic stem cell, primary T cell or CAR-T cell) or population thereof comprising a genome modified to increase expression of a tolerogenic or immunosuppressive factor such as DUX4. In some embodiments, the present disclosure provides a method for altering a cell's genome to provide increased expression of DUX4, including through a exogenous polynucleotide. In some embodiments, the disclosure provides a cell or population thereof comprising exogenously expressed DUX4 proteins. In some embodiments, increased expression of DUX4 suppresses, reduces or eliminates expression of one or more of the following MHC I molecules—HLA-A, HLA-B, and HLA-C. In some embodiments, the exogenous polynucleotide is inserted into at least one allele of the cell using viral transduction, for example, with a vector. In some embodiments, the vector is a pseudotyped, self-inactivating lentiviral vector that carries the exogenous polynucleotide. In some embodiments, the vector is a self-inactivating lentiviral vector pseudotyped with a vesicular stomatitis VSV-G envelope, and which carries the exogenous polynucleotide. In some embodiments, the exogenous polynucleotide is inserted into at least one allele of the cell using viral transduction. In some embodiments, the exogenous polynucleotide is inserted into at least one allele of the cell using a lentivirus based viral vector.

DUX4 is a transcription factor that is active in embryonic tissues and induced pluripotent stem cells, and is silent in normal, healthy somatic tissues (Feng et al., 2015, ELife4; De laco et al., 2017, Nat Genet, 49, 941-945; Hendrickson et al., 2017, Nat Genet, 49, 925-934; Snider et al., 2010, PLoS Genet, e1001181; Whiddon et al., 2017, Nat Genet). DUX4 expression acts to block IFN-gamma mediated induction of MHC class I gene expression (e.g., expression of B2M, HLA-A, HLA-B, and HLA-C). DUX4 expression has been implicated in suppressed antigen presentation by MHC class I (Chew et al., Developmental Cell, 2019, 50, 1-14). DUX4 functions as a transcription factor in the cleavage-stage gene expression (transcriptional) program. Its target genes include, but are not limited to, coding genes, noncoding genes, and repetitive elements.

There are at least two isoforms of DUX4, with the longest isoform comprising the DUX4 C-terminal transcription activation domain. The isoforms are produced by alternative splicing. See, e.g., Geng et al., 2012, Dev Cell, 22, 38-51; Snider et al., 2010, PLoS Genet, e1001181. Active isoforms for DUX4 comprise its N-terminal DNA-binding domains and its C-terminal activation domain. See, e.g., Choi et al., 2016, Nucleic Acid Res, 44, 5161-5173.

It has been shown that reducing the number of CpG motifs of DUX4 decreases silencing of a DUX4 transgene (Jagannathan et al., Human Molecular Genetics, 2016, 25(20):4419-4431). The nucleic acid sequence provided in Jagannathan et al., supra represents a codon altered sequence of DUX4 comprising one or more base substitutions to reduce the total number of CpG sites while preserving the DUX4 protein sequence. The nucleic acid sequence is commercially available from Addgene, Catalog No. 99281.

In many embodiments, at least one or more polynucleotides may be utilized to facilitate the exogenous expression of DUX4 by a cell, e.g., a stem cell, induced pluripotent stem cell, differentiated cell, hematopoietic stem cell, primary T cell or CAR-T cell.

In some embodiments, a suitable gene editing system (e.g., CRISPR/Cas system or any of the gene editing systems described herein) is used to facilitate the insertion of a polynucleotide encoding DUX4, into a genomic locus of the hypoimmunogenic cell. In some cases, the polynucleotide encoding DUX4 is inserted into a safe harbor or target locus, such as but not limited to, an AAVS1, CCR5, CLYBL, ROSA26, SHS231, F3 (CD142), MICA, MICB, LRP1 (CD91), HMGB1, ABO, RHD, FUT1, or KDM5D gene locus. In some embodiments, the polynucleotide encoding DUX4 is inserted into a B2M gene locus, a CIITA gene locus, a TRAC gene locus, or a TRB gene locus. In some embodiments, the polynucleotide encoding DUX4 is inserted into any one of the gene loci depicted in Table 33 or 36 provided herein. In certain embodiments, the polynucleotide encoding DUX4 is operably linked to a promoter.

In some embodiments, the polynucleotide encoding DUX4 is inserted into at least one allele of the T cell using viral transduction. In some embodiments, the polynucleotide encoding DUX4 is inserted into at least one allele of the T cell using a lentivirus based viral vector. In some embodiments, the lentivirus based viral vector is a pseudotyped, self-inactivating lentiviral vector that carries the polynucleotide encoding DUX4. In some embodiments, the lentivirus based viral vector is a self-inactivating lentiviral vector pseudotyped with a vesicular stomatitis VSV-G envelope, and which carries the polynucleotide encoding DUX4.

In some embodiments, the polynucleotide sequence encoding DUX4 comprises a polynucleotide sequence comprising a codon altered nucleotide sequence of DUX4 comprising one or more base substitutions to reduce the total number of CpG sites while preserving the DUX4 protein sequence. In some embodiments, the polynucleotide sequence encoding DUX4 comprising one or more base substitutions to reduce the total number of CpG sites has at least 85% (e.g., 85%, 86%, 87%, 88%, 89%, 90%, 91%, 92%, 93%, 94%, 95%, 96%, 97%, 98%, 99% or 100%) sequence identity to SEQ ID NO:1 of PCT/US2020/44635, filed Jul. 31, 2020. In some embodiments, the polynucleotide sequence encoding DUX4 is SEQ ID NO:1 of PCT/US2020/44635.

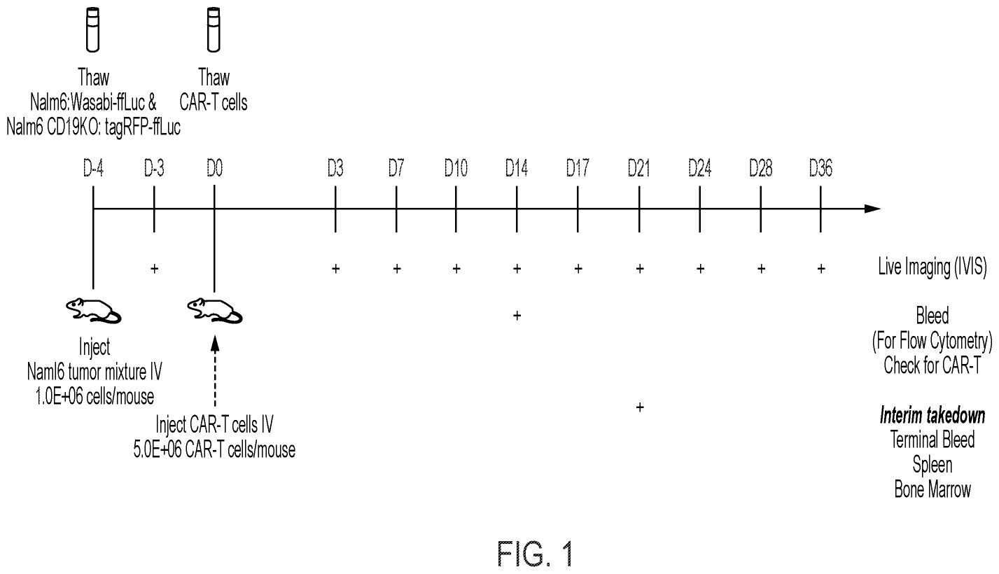

In some embodiments, the polynucleotide sequence encoding DUX4 is a nucleotide sequence encoding a polypeptide sequence having at least 95% (e.g., 95%, 96%, 97%, 98%, 99% or 100%) sequence identity to a sequence selected from a group including SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, and SEQ ID NO:29, as provided in PCT/US2020/44635. In some embodiments, the polynucleotide sequence encoding DUX4 is a nucleotide sequence encoding a polypeptide sequence is selected from a group including SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, and SEQ ID NO:29. Amino acid sequences set forth as SEQ ID NOS:2-29 are shown in FIGS. 1A-1G of PCT/US2020/44635.

In some instances, the DUX4 polypeptide comprises an amino acid sequence having at least 95% sequence identity to the sequence set forth in GenBank Accession No. ACN62209.1 or an amino acid sequence set forth in GenBank Accession No. ACN62209.1. In some instances, the DUX4 polypeptide comprises an amino acid sequence having at least 95% sequence identity to the sequence set forth in NCBI RefSeq No. NP_001280727.1 or an amino acid sequence set forth in NCBI RefSeq No. NP_001280727.1. In some instances, the DUX4 polypeptide comprises an amino acid sequence having at least 95% sequence identity to the sequence set forth in GenBank Accession No. ACP30489.1 or an amino acid sequence set forth in GenBank Accession No. ACP30489.1. In some instances, the DUX4 polypeptide comprises an amino acid sequence having at least 95% sequence identity to the sequence set forth in UniProt No. POCJ85.1 or an amino acid sequence set forth in UniProt No. P0CJ85.1. In some instances, the DUX4 polypeptide comprises an amino acid sequence having at least 95% sequence identity to the sequence set forth in GenBank Accession No. AUA60622.1 or an amino acid sequence set forth in GenBank Accession No. AUA60622.1. In some instances, the DUX4 polypeptide comprises an amino acid sequence having at least 95% sequence identity to the sequence set forth in GenBank Accession No. ADK24683.1 or an amino acid sequence set forth in GenBank Accession No. ADK24683.1. In some instances, the DUX4 polypeptide comprises an amino acid sequence having at least 95% sequence identity to the sequence set forth in GenBank Accession No. ACN62210.1 or an amino acid sequence set forth in GenBank Accession No. ACN62210.1. In some instances, the DUX4 polypeptide comprises an amino acid sequence having at least 95% sequence identity to the sequence set forth in GenBank Accession No. ADK24706.1 or an amino acid sequence set forth in GenBank Accession No. ADK24706.1. In some instances, the DUX4 polypeptide comprises an amino acid sequence having at least 95% sequence identity to the sequence set forth in GenBank Accession No. ADK24685.1 or an amino acid sequence set forth in GenBank Accession No. ADK24685.1. In some instances, the DUX4 polypeptide comprises an amino acid sequence having at least 95% sequence identity to the sequence set forth in GenBank Accession No. ACP30488.1 or an amino acid sequence set forth in GenBank Accession No. ACP30488.1. In some instances, the DUX4 polypeptide comprises an amino acid sequence having at least 95% sequence identity to the sequence set forth in GenBank Accession No. ADK24687.1 or an amino acid sequence set forth in GenBank Accession No. ADK24687.1. In some instances, the DUX4 polypeptide comprises an amino acid sequence having at least 95% sequence identity to the sequence set forth in GenBank Accession No. ACP30487.1 or an amino acid sequence set forth in GenBank Accession No. ACP30487.1. In some instances, the DUX4 polypeptide comprises an amino acid sequence having at least 95% sequence identity to the sequence set forth in GenBank Accession No. ADK24717.1 or an amino acid sequence set forth in GenBank Accession No. ADK24717.1. In some instances, the DUX4 polypeptide comprises an amino acid sequence having at least 95% sequence identity to the sequence set forth in GenBank Accession No. ADK24690.1 or an amino acid sequence set forth in GenBank Accession No. ADK24690.1. In some instances, the DUX4 polypeptide comprises an amino acid sequence having at least 95% sequence identity to the sequence set forth in GenBank Accession No. ADK24689.1 or an amino acid sequence set forth in GenBank Accession No. ADK24689.1. In some instances, the DUX4 polypeptide comprises an amino acid sequence having at least 95% sequence identity to the sequence set forth in GenBank Accession No. ADK24692.1 or an amino acid sequence set forth in GenBank Accession No. ADK24692.1. In some instances, the DUX4 polypeptide comprises an amino acid sequence having at least 95% sequence identity to the sequence set forth in GenBank Accession No. ADK24693.1 or an amino acid sequence of set forth in GenBank Accession No. ADK24693.1. In some instances, the DUX4 polypeptide comprises an amino acid sequence having at least 95% sequence identity to the sequence set forth in GenBank Accession No. ADK24712.1 or an amino acid sequence set forth in GenBank Accession No. ADK24712.1. In some instances, the DUX4 polypeptide comprises an amino acid sequence having at least 95% sequence identity to the sequence set forth in GenBank Accession No. ADK24691.1 or an amino acid sequence set forth in GenBank Accession No. ADK24691.1. In some instances, the DUX4 polypeptide comprises an amino acid sequence having at least 95% sequence identity to the sequence set forth in UniProt No. POCJ87.1 or an amino acid sequence of set forth in UniProt No. POCJ87.1. In some instances, the DUX4 polypeptide comprises an amino acid sequence having at least 95% sequence identity to the sequence set forth in GenBank Accession No. ADK24714.1 or an amino acid sequence set forth in GenBank Accession No. ADK24714.1. In some instances, the DUX4 polypeptide comprises an amino acid sequence having at least 95% sequence identity to the sequence set forth in GenBank Accession No. ADK24684.1 or an amino acid sequence of set forth in GenBank Accession No. ADK24684.1. In some instances, the DUX4 polypeptide comprises an amino acid sequence having at least 95% sequence identity to the sequence set forth in GenBank Accession No. ADK24695.1 or an amino acid sequence set forth in GenBank Accession No. ADK24695.1. In some instances, the DUX4 polypeptide comprises an amino acid sequence having at least 95% sequence identity to the sequence set forth in GenBank Accession No. ADK24699.1 or an amino acid sequence set forth in GenBank Accession No. ADK24699.1. In some instances, the DUX4 polypeptide comprises an amino acid sequence having at least 95% sequence identity to the sequence set forth in NCBI RefSeq No. NP_001768.1 or an amino acid sequence set forth in NCBI RefSeq No. NP_001768. In some instances, the DUX4 polypeptide comprises an amino acid sequence having at least 95% sequence identity to the sequence set forth in NCBI RefSeq No. NP_942088.1 or an amino acid sequence set forth in NCBI RefSeq No. NP_942088.1. In some instances, the DUX4 polypeptide comprises an amino acid sequence having at least 95% sequence identity to SEQ ID NO:28 provided in PCT/US2020/44635 or an amino acid sequence of SEQ ID NO:28 provided in PCT/US2020/44635. In some instances, the DUX4 polypeptide comprises an amino acid sequence having at least 95% sequence identity to SEQ ID NO:29 provided in PCT/US2020/44635 or an amino acid sequence of SEQ ID NO:29 provided in PCT/US2020/44635.

In other embodiments, expression of tolerogenic factors is facilitated using an expression vector. In some embodiments, the expression vector comprises a polynucleotide sequence encoding DUX4 is a codon altered sequence comprising one or more base substitutions to reduce the total number of CpG sites while preserving the DUX4 protein sequence. In some cases, the codon altered sequence of DUX4 comprises SEQ ID NO:1 of PCT/US2020/44635. In some cases, the codon altered sequence of DUX4 is SEQ ID NO:1 of PCT/US2020/44635. In other embodiments, the expression vector comprises a polynucleotide sequence encoding DUX4 comprising SEQ ID NO:1 of PCT/US2020/44635. In some embodiments, the expression vector comprises a polynucleotide sequence encoding a DUX4 polypeptide sequence having at least 95% sequence identity to a sequence selected from a group including SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, and SEQ ID NO:29 of PCT/US2020/44635. In some embodiments, the expression vector comprises a polynucleotide sequence encoding a DUX4 polypeptide sequence selected from a group including SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, and SEQ ID NO:29 of PCT/US2020/44635.

An increase of DUX4 expression can be assayed using known techniques, such as Western blots, ELISA assays, FACS assays, immunoassays, and the like.

### 15. Additional Tolerogenic Factors

In many embodiments, one or more tolerogenic factors can be inserted or reinserted into genome-edited cells to create immune-privileged universal donor cells, such as universal donor stem cells, universal donor T cells, or universal donor cells. In certain embodiments, the engineered CAR-T cells disclosed herein have been further modified to express one or more tolerogenic factors. Exemplary tolerogenic factors include, without limitation, one or more of A20/TNFAIP3, C1-Inhibitor, CCL21, CCL22, CD16, CD16 Fc receptor, CD24, CD27, CD35, CD39, CD46, CD47, CD52, CD55, CD59, CD64, CD200, CR1, CTLA4-Ig, DUX4, FasL, H2-M3, HLA-C, HLA-E, HLA-E heavy chain, HLA-F, HLA-G, IDO1, IL-10, IL15-RF, IL-35, IL-39, MANF, Mfge8, PD-L1, Serpinb9, CCL21, CCL22, B2M-HLA-E, CI inhibitor, and CR1. In some embodiments, the tolerogenic factors are selected from the group consisting of CD200, HLA-G, HLA-E, HLA-C, HLA-E heavy chain, PD-L1, IDO1, CTLA4-Ig, IL-10, IL-35, FasL, Serpinb9, CCL21, CCL22, and Mfge8. In some embodiments, the tolerogenic factors are selected from the group consisting of DUX4, HLA-C, HLA-E, HLA-F, HLA-G, PD-L1, CTLA-4-Ig, C1-inhibitor, and IL-35. In some embodiments, the tolerogenic factors are selected from the group consisting of HLA-C, HLA-E, HLA-F, HLA-G, PD-L1, CTLA-4-Ig, C1-inhibitor, and IL-35. In some embodiments, the tolerogenic factors are selected from a group including A20/TNFAIP3, C1-Inhibitor, CCL21, CCL22, CD16, CD16 Fc receptor, CD24, CD27, CD35, CD39, CD46, CD47, CD52, CD55, CD59, CD64, CD200, CR1, CTLA4-Ig, DUX4, FasL, H2-M3, HLA-C, HLA-E, HLA-E heavy chain, HLA-F, HLA-G, IDO1, IL-10, IL15-RF, IL-35, IL-39, MANF, Mfge8, PD-L1, Serpinb9, CCL21, CCL22, B2M-HLA-E, CI inhibitor, and CR1.

In some embodiments, the polynucleotide encoding the one or more tolerogenic factors is inserted into at least one allele of the T cell using viral transduction. In some embodiments, the polynucleotide encoding the one or more tolerogenic factors is inserted into at least one allele of the T cell using a lentivirus based viral vector. In some embodiments, the lentivirus based viral vector is a pseudotyped, self-inactivating lentiviral vector that carries the polynucleotide encoding the one or more tolerogenic factors. In some embodiments, the lentivirus based viral vector is a self-inactivating lentiviral vector pseudotyped with a vesicular stomatitis VSV-G envelope, and which carries the polynucleotide encoding the one or more tolerogenic factors.

Useful genomic, polynucleotide and polypeptide information about human CD27 (which is also known as CD27L receptor, Tumor Necrosis Factor Receptor Superfamily Member 7, TNFSF7, T Cell Activation Antigen S152, Tp55, and T14) are provided in, for example, the GeneCard Identifier GC12P008144, HGNC No. 11922, NCBI Gene ID 939, Uniprot No. P26842, and NCBI RefSeq Nos. NM_001242.4 and NP_001233.1.

Useful genomic, polynucleotide and polypeptide information about human CD46 are provided in, for example, the GeneCard Identifier GC01P207752, HGNC No. 6953, NCBI Gene ID 4179, Uniprot No. P15529, and NCBI RefSeq Nos. NM_002389.4, NM_153826.3, NM_172350.2, NM_172351.2, NM_172352.2 NP_758860.1, NM_172353.2, NM_172359.2, NM_172361.2, NP_002380.3, NP_722548.1, NP_758860.1, NP_758861.1, NP_758862.1, NP_758863.1, NP_758869.1, and NP_758871.1.

Useful genomic, polynucleotide and polypeptide information about human CD55 (also known as complement decay-accelerating factor) are provided in, for example, the GeneCard Identifier GC01P207321, HGNC No. 2665, NCBI Gene ID 1604, Uniprot No. P08174, and NCBI RefSeq Nos. NM_000574.4, NM_001114752.2, NM_001300903.1, NM_001300904.1, NP_000565.1, NP_001108224.1, NP_001287832.1, and NP_001287833.1.

Useful genomic, polynucleotide and polypeptide information about human CD59 are provided in, for example, the GeneCard Identifier GC11M033704, HGNC No. 1689, NCBI Gene ID 966, Uniprot No. P13987, and NCBI RefSeq Nos. NP_000602.1, NM_000611.5, NP_001120695.1, NM_001127223.1, NP_001120697.1, NM_001127225.1, NP_001120698.1, NM_001127226.1, NP_001120699.1, NM_001127227.1, NP_976074.1, NM_203329.2, NP_976075.1, NM_203330.2, NP_976076.1, and NM_203331.2.

Useful genomic, polynucleotide and polypeptide information about human CD200 are provided in, for example, the GeneCard Identifier GC03P112332, HGNC No. 7203, NCBI Gene ID 4345, Uniprot No. P41217, and NCBI RefSeq Nos. NP_001004196.2, NM_001004196.3, NP_001305757.1, NM_001318828.1, NP_005935.4, NM_005944.6, XP_005247539.1, and XM_005247482.2.

Useful genomic, polynucleotide and polypeptide information about human HLA-C are provided in, for example, the GeneCard Identifier GC06M031272, HGNC No. 4933, NCBI Gene ID 3107, Uniprot No. P10321, and NCBI RefSeq Nos. NP_002108.4 and NM_002117.5.

Useful genomic, polynucleotide and polypeptide information about human HLA-E are provided in, for example, the GeneCard Identifier GC06P047281, HGNC No. 4962, NCBI Gene ID 3133, Uniprot No. P13747, and NCBI RefSeq Nos. NP_005507.3 and NM_005516.5.

Useful genomic, polynucleotide and polypeptide information about human HLA-G are provided in, for example, the GeneCard Identifier GC06P047256, HGNC No. 4964, NCBI Gene ID 3135, Uniprot No. P17693, and NCBI RefSeq Nos. NP_002118.1 and NM_002127.5.

Useful genomic, polynucleotide and polypeptide information about human PD-L1 or CD274 are provided in, for example, the GeneCard Identifier GC09P005450, HGNC No. 17635, NCBI Gene ID 29126, Uniprot No. Q9NZQ7, and NCBI RefSeq Nos. NP_001254635.1, NM_001267706.1, NP_054862.1, and NM_014143.3.

Useful genomic, polynucleotide and polypeptide information about human IDO1 are provided in, for example, the GeneCard Identifier GC08P039891, HGNC No. 6059, NCBI Gene ID 3620, Uniprot No. P14902, and NCBI RefSeq Nos. NP_002155.1 and NM_002164.5.

Useful genomic, polynucleotide and polypeptide information about human IL-10 are provided in, for example, the GeneCard Identifier GC01M206767, HGNC No. 5962, NCBI Gene ID 3586, Uniprot No. P22301, and NCBI RefSeq Nos. NP_000563.1 and NM_000572.2.

Useful genomic, polynucleotide and polypeptide information about human Fas ligand (which is known as FasL, FASLG, CD178, TNFSF6, and the like) are provided in, for example, the GeneCard Identifier GC01P172628, HGNC No. 11936, NCBI Gene ID 356, Uniprot No. P48023, and NCBI RefSeq Nos. NP_000630.1, NM_000639.2, NP_001289675.1, and NM_001302746.1.

Useful genomic, polynucleotide and polypeptide information about human CCL21 are provided in, for example, the GeneCard Identifier GC09M034709, HGNC No. 10620, NCBI Gene ID 6366, Uniprot No. 000585, and NCBI RefSeq Nos. NP_002980.1 and NM_002989.3.

Useful genomic, polynucleotide and polypeptide information about human CCL22 are provided in, for example, the GeneCard Identifier GC16P057359, HGNC No. 10621, NCBI Gene ID 6367, Uniprot No. 000626, and NCBI RefSeq Nos. NP_002981.2, NM_002990.4, XP_016879020.1, and XM_017023531.1.

Useful genomic, polynucleotide and polypeptide information about human Mfge8 are provided in, for example, the GeneCard Identifier GC15M088898, HGNC No. 7036, NCBI Gene ID 4240, Uniprot No. Q08431, and NCBI RefSeq Nos. NP_001108086.1, NM_001114614.2, NP_001297248.1, NM_001310319.1, NP_001297249.1, NM_001310320.1, NP_001297250.1, NM_001310321.1, NP_005919.2, and NM_005928.3.

Useful genomic, polynucleotide and polypeptide information about human SerpinB9 are provided in, for example, the GeneCard Identifier GC06M002887, HGNC No. 8955, NCBI Gene ID 5272, Uniprot No. P50453, and NCBI RefSeq Nos. NP_004146.1, NM_004155.5, XP_005249241.1, and XM_005249184.4.

Methods for modulating expression of genes and factors (proteins) include genome editing technologies, RNA or protein expression technologies, and the like. For all of these technologies, well known recombinant techniques are used, to generate recombinant nucleic acids as outlined herein.

In some embodiments, the cells (e.g., stem cell, induced pluripotent stem cell, differentiated cell, hematopoietic stem cell, primary T cell or CAR-T cell) possess genetic modifications that inactivate the B2M and CIITA genes and express a plurality of exogenous polypeptides selected from the group including CD47 and DUX4, CD47 and CD24, CD47 and CD27, CD47 and CD46, CD47 and CD55, CD47 and CD59, CD47 and CD200, CD47 and HLA-C, CD47 and HLA-E, CD47 and HLA-E heavy chain, CD47 and HLA-G, CD47 and PD-L1, CD47 and IDO1, CD47 and CTLA4-Ig, CD47 and C1-Inhibitor, CD47 and IL-10, CD47 and IL-35, CD47 and IL-39, CD47 and FasL, CD47 and CCL21, CD47 and CCL22, CD47 and Mfge8, and CD47 and Serpinb9, and any combination thereof. In some instances, such cells also possess a genetic modification that inactivates the CD142 gene.

In some instances, a gene editing system such as the CRISPR/Cas system is used to facilitate the insertion of tolerogenic factors, such as the tolerogenic factors into a safe harbor or target locus, such as the AAVS1 locus, to actively inhibit immune rejection. In some instances, the tolerogenic factors are inserted into a safe harbor or target locus using an expression vector. In some embodiments, the safe harbor or target locus is an AAVS1, CCR5, CLYBL, ROSA26, SHS231, F3 (also known as CD142), MICA, MICB, LRP1 (also known as CD91), HMGB1, ABO, RHD, FUT1, or KDMSD gene locus.

In some embodiments, expression of a target gene (e.g., DUX4, CD47, or another tolerogenic factor gene) is increased by expression of fusion protein or a protein complex containing (1) a site-specific binding domain specific for the endogenous target gene (e.g., DUX4, CD47, or another tolerogenic factor gene) and (2) a transcriptional activator.

In some embodiments, the regulatory factor is comprised of a site specific DNA-binding nucleic acid molecule, such as a guide RNA (gRNA). In some embodiments, the method is achieved by site specific DNA-binding targeted proteins, such as zinc finger proteins (ZFP) or fusion proteins containing ZFP, which are also known as zinc finger nucleases (ZFNs).

In some embodiments, the regulatory factor comprises a site-specific binding domain, such as using a DNA binding protein or DNA-binding nucleic acid, which specifically binds to or hybridizes to the gene at a targeted region. In some embodiments, the provided polynucleotides or polypeptides are coupled to or complexed with a site-specific nuclease, such as a modified nuclease. For example, in some embodiments, the administration is effected using a fusion comprising a DNA-targeting protein of a modified nuclease, such as a meganuclease or an RNA-guided nuclease such as a clustered regularly interspersed short palindromic nucleic acid (CRISPR)-Cas system, such as CRISPR-Cas9 system. In some embodiments, the nuclease is modified to lack nuclease activity. In some embodiments, the modified nuclease is a catalytically dead dCas9.

In some embodiments, the site specific binding domain may be derived from a nuclease. For example, the recognition sequences of homing endonucleases and meganucleases such as I-Scel, I-Ceul, PI-Pspl, PI-Sce, I-ScelV, I-Csml, I-Panl, I-Scell, I-Ppol, I-ScellI, I-Crel, I-Tevl, I-Tevil and I-Tevill. See also U.S. Pat. Nos. 5,420,032; 6,833,252; Belfort et al., (1997) Nucleic Acids Res. 25:3379-3388; Dujon et al., (1989) Gene 82:115-118; Perler et al, (1994) Nucleic Acids Res. 22, 1125-1127; Jasin (1996) Trends Genet. 12:224-228; Gimble et al., (1996) J. Mol. Biol. 263:163-180; Argast et al, (1998) J. Mol. Biol. 280:345-353 and the New England Biolabs catalogue. In addition, the DNA-binding specificity of homing endonucleases and meganucleases can be engineered to bind non-natural target sites. See, for example, Chevalier et al, (2002) Molec. Cell 10:895-905; Epinat et al, (2003) Nucleic Acids Res. 31:2952-2962; Ashworth et al, (2006) Nature 441:656-659; Paques et al, (2007) Current Gene Therapy 7:49-66; U.S. Patent Publication No. 2007/0117128.

Zinc finger, TALE, and CRISPR system binding domains can be “engineered” to bind to a predetermined nucleotide sequence, for example via engineering (altering one or more amino acids) of the recognition helix region of a naturally occurring zinc finger or TALE protein. Engineered DNA binding proteins (zinc fingers or TALEs) are proteins that are non-naturally occurring. Rational criteria for design include application of substitution rules and computerized algorithms for processing information in a database storing information of existing ZFP and/or TALE designs and binding data. See, for example, U.S. Pat. Nos. 6,140,081; 6,453,242; and 6,534,261; see also WO 98/53058; WO 98/53059; WO 98/53060; WO 02/016536 and WO 03/016496 and U.S. Publication No. 20110301073.

In some embodiments, the site-specific binding domain comprises one or more zinc-finger proteins (ZFPs) or domains thereof that bind to DNA in a sequence-specific manner. A ZFP or domain thereof is a protein or domain within a larger protein that binds DNA in a sequence-specific manner through one or more zinc fingers, regions of amino acid sequence within the binding domain whose structure is stabilized through coordination of a zinc ion.

Among the ZFPs are artificial ZFP domains targeting specific DNA sequences, typically 9-18 nucleotides long, generated by assembly of individual fingers. ZFPs include those in which a single finger domain is approximately 30 amino acids in length and contains an alpha helix containing two invariant histidine residues coordinated through zinc with two cysteines of a single beta turn, and having two, three, four, five, or six fingers. Generally, sequence-specificity of a ZFP may be altered by making amino acid substitutions at the four helix positions (−1, 2, 3 and 6) on a zinc finger recognition helix. Thus, in some embodiments, the ZFP or ZFP-containing molecule is non-naturally occurring, e.g., is engineered to bind to a target site of choice. See, for example, Beerli et al. (2002) Nature Biotechnol. 20:135-141; Pabo et al. (2001) Ann. Rev. Biochem. 70:313-340; Isalan et al. (2001) Nature Biotechnol. 19:656-660; Segal et al. (2001) Curr. Opin. Biotechnol. 12:632-637; Choo et al. (2000) Curr. Opin. Struct. Biol. 10:411-416; U.S. Pat. Nos. 6,453,242; 6,534,261; 6,599,692; 6,503,717; 6,689,558; 7,030,215; 6,794,136; 7,067,317; 7,262,054; 7,070,934; 7,361,635; 7,253,273; and U.S. Patent Publication Nos. 2005/0064474; 2007/0218528; 2005/0267061, all incorporated herein by reference in their entireties.

Many gene-specific engineered zinc fingers are available commercially. For example, Sangamo Biosciences (Richmond, CA, USA) has developed a platform (CompoZr) for zinc-finger construction in partnership with Sigma-Aldrich (St. Louis, MO, USA), allowing investigators to bypass zinc-finger construction and validation altogether, and provides specifically targeted zinc fingers for thousands of proteins (Gaj et al., Trends in Biotechnology, 2013, 31(7), 397-405). In some embodiments, commercially available zinc fingers are used or are custom designed.

In some embodiments, the site-specific binding domain comprises a naturally occurring or engineered (non-naturally occurring) transcription activator-like protein (TAL) DNA binding domain, such as in a transcription activator-like protein effector (TALE) protein, See, e.g., U.S. Patent Publication No. 20110301073, incorporated by reference in its entirety herein.

In some embodiments, the site-specific binding domain is derived from the CRISPR/Cas system. In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g., tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system, or a “targeting sequence”), and/or other sequences and transcripts from a CRISPR locus.

In general, a guide sequence includes a targeting domain comprising a polynucleotide sequence having sufficient complementarity with a target polynucleotide sequence to hybridize with the target sequence and direct sequence-specific binding of the CRISPR complex to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more. In some examples, the targeting domain of the gRNA is complementary, e.g., at least 80, 85, 90, 95, 98 or 99% complementary, e.g., fully complementary, to the target sequence on the target nucleic acid.

In some embodiments, the target site is upstream of a transcription initiation site of the target gene. In some embodiments, the target site is adjacent to a transcription initiation site of the gene. In some embodiments, the target site is adjacent to an RNA polymerase pause site downstream of a transcription initiation site of the gene.

In some embodiments, the targeting domain is configured to target the promoter region of the target gene to promote transcription initiation, binding of one or more transcription enhancers or activators, and/or RNA polymerase. One or more gRNA can be used to target the promoter region of the gene. In some embodiments, one or more regions of the gene can be targeted. In certain aspects, the target sites are within 600 base pairs on either side of a transcription start site (TSS) of the gene.

It is within the level of a skilled artisan to design or identify a gRNA sequence that is or comprises a sequence targeting a gene, including the exon sequence and sequences of regulatory regions, including promoters and activators. A genome-wide gRNA database for CRISPR genome editing is publicly available, which contains exemplary single guide RNA (sgRNA) target sequences in constitutive exons of genes in the human genome or mouse genome (see e.g., genescript.com/gRNA-database.html; see also, Sanjana et al. (2014) Nat. Methods, 11:783-4; www.e-crisp.org/E-CRISP/; crispr.mit.edu/). In some embodiments, the gRNA sequence is or comprises a sequence with minimal off-target binding to a non-target gene.

In some embodiments, the regulatory factor further comprises a functional domain, e.g., a transcriptional activator.

In some embodiments, the transcriptional activator is or contains one or more regulatory elements, such as one or more transcriptional control elements of a target gene, whereby a site-specific domain as provided above is recognized to drive expression of such gene. In some embodiments, the transcriptional activator drives expression of the target gene. In some cases, the transcriptional activator, can be or contain all or a portion of an heterologous transactivation domain. For example, in some embodiments, the transcriptional activator is selected from Herpes simplex-derived transactivation domain, Dnmt3a methyltransferase domain, p65, VP16, and VP64.

In some embodiments, the regulatory factor is a zinc finger transcription factor (ZF-TF). In some embodiments, the regulatory factor is VP64-p65-Rta (VPR).

In certain embodiments, the regulatory factor further comprises a transcriptional regulatory domain. Common domains include, e.g., transcription factor domains (activators, repressors, co-activators, co-repressors), silencers, oncogenes (e.g., myc, jun, fos, myb, max, mad, rel, ets, bcl, myb, mos family members etc.); DNA repair enzymes and their associated factors and modifiers; DNA rearrangement enzymes and their associated factors and modifiers; chromatin associated proteins and their modifiers (e.g., kinases, acetylases and deacetylases); and DNA modifying enzymes (e.g., methyltransferases such as members of the DNMT family (e.g., DNMT1, DNMT3A, DNMT3B, DNMT3L, etc., topoisomerases, helicases, ligases, kinases, phosphatases, polymerases, endonucleases) and their associated factors and modifiers. See, e.g., U.S. Publication No. 2013/0253040, incorporated by reference in its entirety herein.

Suitable domains for achieving activation include the HSV VP 16 activation domain (see, e.g., Hagmann et al, J. Virol. 71, 5952-5962 (197)) nuclear hormone receptors (see, e.g., Torchia et al., Curr. Opin. Cell. Biol. 10:373-383 (1998)); the p65 subunit of nuclear factor kappa B (Bitko & Bank, J. Virol. 72:5610-5618 (1998) and Doyle & Hunt, Neuroreport 8:2937-2942 (1997)); Liu et al., Cancer Gene Ther. 5:3-28 (1998)), or artificial chimeric functional domains such as VP64 (Beerli et al., (1998) Proc. Natl. Acad. Sci. USA 95:14623-33), and degron (Molinari et al., (1999) EMBO J. 18, 6439-6447). Additional exemplary activation domains include, Oct 1, Oct-2A, Spl, AP-2, and CTF1 (Seipel etal, EMBOJ. 11, 4961-4968 (1992) as well as p300, CBP, PCAF, SRC1 PvALF, AtHD2A and ERF-2. See, for example, Robyr et al, (2000) Mol. Endocrinol. 14:329-347; Collingwood et al, (1999) J. Mol. Endocrinol 23:255-275; Leo et al, (2000) Gene 245:1-11; Manteuffel-Cymborowska (1999) Acta Biochim. Pol. 46:77-89; McKenna et al, (1999) J. Steroid Biochem. Mol. Biol. 69:3-12; Malik et al, (2000) Trends Biochem. Sci. 25:277-283; and Lemon et al, (1999) Curr. Opin. Genet. Dev. 9:499-504. Additional exemplary activation domains include, but are not limited to, OsGAI, HALF-1, CI, AP1, ARF-5, -6,-1, and -8, CPRF1, CPRF4, MYC-RP/GP, and TRAB1, See, for example, Ogawa et al, (2000) Gene 245:21-29; Okanami et al, (1996) Genes Cells 1:87-99; Goff et al, (1991) Genes Dev. 5:298-309; Cho et al, (1999) Plant Mol Biol 40:419-429; Ulmason et al, (1999) Proc. Natl. Acad. Sci. USA 96:5844-5849; Sprenger-Haussels et al, (2000) Plant J. 22:1-8; Gong et al, (1999) Plant Mol. Biol. 41:33-44; and Hobo et al., (1999) Proc. Natl. Acad. Sci. USA 96:15,348-15,353.

Exemplary repression domains that can be used to make genetic repressors include, but are not limited to, KRAB A/B, KOX, TGF-beta-inducible early gene (TIEG), v-erbA, SID, MBD2, MBD3, members of the DNMT family (e.g., DNMT1, DNMT3A, DNMT3B, DNMT3L, etc.), Rb, and MeCP2. See, for example, Bird et al, (1999) Cell 99:451-454; Tyler et al, (1999) Cell 99:443-446; Knoepfler et al, (1999) Cell 99:447-450; and Robertson et al, (2000) Nature Genet. 25:338-342. Additional exemplary repression domains include, but are not limited to, ROM2 and AtHD2A. See, for example, Chem et al, (1996) Plant Cell 8:305-321; and Wu et al, (2000) Plant J. 22:19-27.

In some instances, the domain is involved in epigenetic regulation of a chromosome. In some embodiments, the domain is a histone acetyltransferase (HAT), e.g., type-A, nuclear localized such as MYST family members MOZ, Ybf2/Sas3, MOF, and Tip60, GNAT family members Gcn5 or pCAF, the p300 family members CBP, p300 or Rtt109 (Bemdsen and Denu (2008) Curr Opin Struct Biol 18(6):682-689). In other instances the domain is a histone deacetylase (HD AC) such as the class I (HDAC-1, 2, 3, and 8), class II (HDAC IIA (HDAC-4, 5, 7 and 9), HD AC IIB (HDAC 6 and 10)), class IV (HDAC-I 1), class Ill (also known as sirtuins (SIRTs); SIRT1-7) (see Mottamal et al., (2015) Molecules 20(3):3898-3941). Another domain that is used in some embodiments is a histone phosphorylase or kinase, where examples include MSK1, MSK2, ATR, ATM, DNA-PK, Bubl, VprBP, IKK-a, PKCpi, Dik/Zip, JAK2, PKC5, WSTF and CK2. In some embodiments, a methylation domain is used and may be chosen from groups such as Ezh2, PRMT1/6, PRMT5/7, PRMT 2/6, CARM1, set7/9, MLL, ALL-1, Suv 39h, G9a, SETDB1, Ezh2, Set2, Dotl, PRMT 1/6, PRMT 5/7, PR-Set7 and Suv4-20h, Domains involved in sumoylation and biotinylation (Lys9, 13, 4, 18 and 12) may also be used in some embodiments (review see Kousarides (2007) Cell 128:693-705).

Fusion molecules are constructed by methods of cloning and biochemical conjugation that are well known to those of skill in the art. Fusion molecules comprise a DNA-binding domain and a functional domain (e.g., a transcriptional activation or repression domain). Fusion molecules also optionally comprise nuclear localization signals (such as, for example, that from the SV40 medium T-antigen) and epitope tags (such as, for example, FLAG and hemagglutinin). Fusion proteins (and nucleic acids encoding them) are designed such that the translational reading frame is preserved among the components of the fusion.

Fusions between a polypeptide component of a functional domain (or a functional fragment thereof) on the one hand, and a non-protein DNA-binding domain (e.g., antibiotic, intercalator, minor groove binder, nucleic acid) on the other, are constructed by methods of biochemical conjugation known to those of skill in the art. See, for example, the Pierce Chemical Company (Rockford, IL) Catalogue. Methods and compositions for making fusions between a minor groove binder and a polypeptide have been described. Mapp et al, (2000) Proc. Natl. Acad. Sci. USA 97:3930-3935. Likewise, CRISPR/Cas TFs and nucleases comprising a sgRNA nucleic acid component in association with a polypeptide component function domain are also known to those of skill in the art and detailed herein.

In some embodiments, the present disclosure provides a cell (e.g., a primary T cell and a hypoimmunogenic stem cell and derivative thereof) or population thereof comprising a genome in which the cell genome has been modified to express CD47. In some embodiments, the present disclosure provides a method for altering a cell genome to express CD47. In certain embodiments, at least one ribonucleic acid or at least one pair of ribonucleic acids may be utilized to facilitate the insertion of CD47 into a cell line. In certain embodiments, the at least one ribonucleic acid or the at least one pair of ribonucleic acids is selected from the group consisting of SEQ ID NOS:200784-231885 of Table 29 of WO2016183041, which is herein incorporated by reference.

In some embodiments, the present disclosure provides a cell (e.g., a primary T cell and a hypoimmunogenic stem cell and derivative thereof) or population thereof comprising a genome in which the cell genome has been modified to express HLA-C. In some embodiments, the present disclosure provides a method for altering a cell genome to express HLA-C. In certain embodiments, at least one ribonucleic acid or at least one pair of ribonucleic acids may be utilized to facilitate the insertion of HLA-C into a cell line. In certain embodiments, the at least one ribonucleic acid or the at least one pair of ribonucleic acids is selected from the group consisting of SEQ ID NOS:3278-5183 of Table 10 of WO2016183041, which is herein incorporated by reference.

In some embodiments, the present disclosure provides a cell (e.g., a primary T cell and a hypoimmunogenic stem cell and derivative thereof) or population thereof comprising a genome in which the cell genome has been modified to express HLA-E. In some embodiments, the present disclosure provides a method for altering a cell genome to express HLA-E. In certain embodiments, at least one ribonucleic acid or at least one pair of ribonucleic acids may be utilized to facilitate the insertion of HLA-E into a cell line. In certain embodiments, the at least one ribonucleic acid or the at least one pair of ribonucleic acids is selected from the group consisting of SEQ ID NOS:189859-193183 of Table 19 of WO2016183041, which is herein incorporated by reference.

In some embodiments, the present disclosure provides a cell (e.g., a primary T cell and a hypoimmunogenic stem cell and derivative thereof) or population thereof comprising a genome in which the cell genome has been modified to express HLA-F. In some embodiments, the present disclosure provides a method for altering a cell genome to express HLA-F. In certain embodiments, at least one ribonucleic acid or at least one pair of ribonucleic acids may be utilized to facilitate the insertion of HLA-F into a cell line. In certain embodiments, the at least one ribonucleic acid or the at least one pair of ribonucleic acids is selected from the group consisting of SEQ ID NOS: 688808-399754 of Table 45 of WO2016183041, which is herein incorporated by reference.

In some embodiments, the present disclosure provides a cell (e.g., a primary T cell and a hypoimmunogenic stem cell and derivative thereof) or population thereof comprising a genome in which the cell genome has been modified to express HLA-G. In some embodiments, the present disclosure provides a method for altering a cell genome to express HLA-G. In certain embodiments, at least one ribonucleic acid or at least one pair of ribonucleic acids may be utilized to facilitate the insertion of HLA-G into a stem cell line. In certain embodiments, the at least one ribonucleic acid or the at least one pair of ribonucleic acids is selected from the group consisting of SEQ ID NOS:188372-189858 of Table 18 of WO2016183041, which is herein incorporated by reference.

In some embodiments, the present disclosure provides a cell (e.g., a primary T cell and a hypoimmunogenic stem cell and derivative thereof) or population thereof comprising a genome in which the cell genome has been modified to express PD-L1. In some embodiments, the present disclosure provides a method for altering a cell genome to express PD-L1. In certain embodiments, at least one ribonucleic acid or at least one pair of ribonucleic acids may be utilized to facilitate the insertion of PD-L1 into a stem cell line. In certain embodiments, the at least one ribonucleic acid or the at least one pair of ribonucleic acids is selected from the group consisting of SEQ ID NOS:193184-200783 of Table 21 of WO2016183041, which is herein incorporated by reference.

In some embodiments, the present disclosure provides a cell (e.g., a primary T cell and a hypoimmunogenic stem cell and derivative thereof) or population thereof comprising a genome in which the cell genome has been modified to express CTLA4-Ig. In some embodiments, the present disclosure provides a method for altering a cell genome to express CTLA4-Ig. In certain embodiments, at least one ribonucleic acid or at least one pair of ribonucleic acids may be utilized to facilitate the insertion of CTLA4-Ig into a stem cell line. In certain embodiments, the at least one ribonucleic acid or the at least one pair of ribonucleic acids is selected from any one disclosed in WO2016183041, including the sequence listing.

In some embodiments, the present disclosure provides a cell (e.g., a primary T cell and a hypoimmunogenic stem cell and derivative thereof) or population thereof comprising a genome in which the cell genome has been modified to express CI-inhibitor. In some embodiments, the present disclosure provides a method for altering a cell genome to express CI-inhibitor. In certain embodiments, at least one ribonucleic acid or at least one pair of ribonucleic acids may be utilized to facilitate the insertion of CI-inhibitor into a stem cell line. In certain embodiments, the at least one ribonucleic acid or the at least one pair of ribonucleic acids is selected from any one disclosed in WO2016183041, including the sequence listing.

In some embodiments, the present disclosure provides a cell (e.g., a primary T cell and a hypoimmunogenic stem cell and derivative thereof) or population thereof comprising a genome in which the cell genome has been modified to express IL-35. In some embodiments, the present disclosure provides a method for altering a cell genome to express IL-35. In certain embodiments, at least one ribonucleic acid or at least one pair of ribonucleic acids may be utilized to facilitate the insertion of IL-35 into a stem cell line. In certain embodiments, the at least one ribonucleic acid or the at least one pair of ribonucleic acids is selected from any one disclosed in WO2016183041, including the

### SEQUENCE LISTING

In some embodiments, the tolerogenic factors are expressed in a cell using an expression vector. In some embodiments, the tolerogenic factors are introduced to the cell using a viral expression vector that mediates integration of the tolerogenic factor sequence into the genome of the cell. For example, the expression vector for expressing CD47 in a cell comprises a polynucleotide sequence encoding CD47. The expression vector can be an inducible expression vector. The expression vector can be a viral vector, such as but not limited to, a lentiviral vector. In some embodiments, the tolerogenic factors are introduced into the cells using fusogen-mediated delivery or a transposase system selected from the group consisting of conditional or inducible transposases, conditional or inducible PiggyBac transposons, conditional or inducible Sleeping Beauty (SB11) transposons, conditional or inducible Mos1 transposons, and conditional or inducible Tol2 transposons.

In some embodiments, the present disclosure provides a cell (e.g., a primary T cell and a hypoimmunogenic stem cell and derivative thereof) or population thereof comprising a genome in which the cell genome has been modified to express any one of the polypeptides selected from the group consisting of HLA-A, HLA-B, HLA-C, RFX-ANK, CIITA, NFY-A, NLRC5, B2M, RFX5, RFX-AP, HLA-G, HLA-E, NFY-B, PD-L1, NFY-C, IRF1, TAP1, GITR, 4-1BB, CD28, B7-1, CD47, B7-2, OX40, CD27, HVEM, SLAM, CD226, ICOS, LAG3, TIGIT, TIM3, CD160, BTLA, CD244, LFA-1, ST2, HLA-F, CD30, B7-H3, VISTA, TLT, PD-L2, CD58, CD2, HELIOS, and IDO1. In some embodiments, the present disclosure provides a method for altering a cell genome to express any one of the polypeptides selected from the group consisting of HLA-A, HLA-B, HLA-C, RFX-ANK, CIITA, NFY-A, NLRC5, B2M, RFX5, RFX-AP, HLA-G, HLA-E, NFY-B, PD-L1, NFY-C, IRF1, TAP1, GITR, 4-1BB, CD28, B7-1, CD47, B7-2, OX40, CD27, HVEM, SLAM, CD226, ICOS, LAG3, TIGIT, TIM3, CD160, BTLA, CD244, LFA-1, ST2, HLA-F, CD30, B7-H3, VISTA, TLT, PD-L2, CD58, CD2, HELIOS, and IDO1. In certain embodiments, at least one ribonucleic acid or at least one pair of ribonucleic acids may be utilized to facilitate the insertion of the selected polypeptide into a stem cell line. In certain embodiments, the at least one ribonucleic acid or the at least one pair of ribonucleic acids is selected from any one disclosed in Appendices 1-47 and the sequence listing of WO2016183041, the disclosure is incorporated herein by references.

In some embodiments, a suitable gene editing system (e.g., CRISPR/Cas system or any of the gene editing systems described herein) is used to facilitate the insertion of a polynucleotide encoding a tolerogenic factor, into a genomic locus of the hypoimmunogenic cell. In some cases, the polynucleotide encoding the tolerogenic factor is inserted into a safe harbor or target locus, such as but not limited to, an AAVS1, CCR5, CLYBL, ROSA26, SHS231, F3 (CD142), MICA, MICB, LRP1 (CD91), HMGB1, ABO, RHD, FUT1, or KDM5D gene locus. In some embodiments, the polynucleotide encoding the tolerogenic factor is inserted into a B2M gene locus, a CIITA gene locus, a TRAC gene locus, or a TRB gene locus. In some embodiments, the polynucleotide encoding the tolerogenic factor is inserted into any one of the gene loci depicted in Table 33 or 36 provided herein. In certain embodiments, the polynucleotide encoding the tolerogenic factor is operably linked to a promoter.

In some embodiments, the cells are engineered to expresses an increased amount of one or more of A20/TNFAIP3, C1-Inhibitor, CCL21, CCL22, CD16, CD16 Fc receptor, CD24, CD27, CD35, CD39, CD46, CD47, CD52, CD55, CD59, CD64, CD200, CR1, CTLA4-Ig, DUX4, FasL, H2-M3, HLA-C, HLA-E, HLA-E heavy chain, HLA-F, HLA-G, IDO1, IL-10, IL15-RF, IL-35, IL-39, MANF, Mfge8, PD-L1, Serpinb9, CCL21, CCL22, B2M-HLA-E, C1 inhibitor, CR1, or a combination thereof relative to a cell of the same cell type that does not comprise the modifications.

### D. Safety Switch

In some embodiments, an engineered cell provided herein comprises a safety switch. In some embodiments, a safety switch is included in a vector or inserted in a gene locus and allows for controlled killing of the cells in the event of cytotoxicity or other negative consequences to the recipient, thus increasing the safety of cell-based therapies, including those using tolerogenic factors. Detailed descriptions of exemplary safety switches can be found, for example, in WO2021/146627, PCT Application No. PCT/US21/54326 filed on Oct. 9, 2021, and US Provisional Application Nos. 63/222,954 filed on Jul. 16, 2021, 63/282,961 filed on Nov. 24, 2021; the disclosures such as the sequence listings, specifications, and figures are herein incorporated in their entirety.

In some embodiments, a safety switch is included in a vector. In certain embodiments, a vector may comprise one or more expression cassettes each comprising a nucleotide sequence encoding a safety switch. A safety switch can be used, e.g., in a polycistronic vector of the present technology to induce death or apoptosis of host cells containing the polycistronic vector, for example if the cells grow and divide in an undesired manner or cause excessive toxicity to the host. Thus, the use of safety switches enables one to conditionally eliminate aberrant cells in vivo and can be a critical step for the application of cell therapies in the clinic. Safety switches and their uses thereof are disclosed in, for example, Duzgune, Origins of Suicide Gene Therapy (2019); Duzgune (eds), Suicide Gene Therapy. Methods in Molecular Biology, vol. 1895 (Humana Press, New York, NY) (for HSVtk, cytosine deaminase, nitroreductase, purine nucleoside phosphorylase, and horseradish peroxidase); Zhou and Brenner, Exp Hematol 44(11):1013-1019 (2016) (for iCaspase9); Wang et al., Blood 18(5):1255-1263 (2001) (for huEGFR); U.S. Patent Application Publication No. 20180002397 (for HER1); and Philip et al., Blood124(8):1277-1287 (2014) (for RQR8).

In some embodiments, a safety switch can cause cell death in a controlled manner, for example, in the presence of a drug or prodrug or upon activation by a selective exogenous compound. In some embodiments, expression of a safety switch is regulated either by a promoter of the vector, in the case of genomic location-independent transcriptional regulation, or by an endogenous promoter, in the case of site-specific integration of the construct into target gene locus.

In some embodiments, a safety switch comprises a herpes simplex virus thymidine kinase (HSVtk), cytosine deaminase (CyD), nitroreductase (NTR), purine nucleoside phosphorylase (PNP), horseradish peroxidase, inducible caspase 9 (iCasp9), rapamycin-activated caspase such as rapaCasp9, CCR4, CD16, CD19, CD20, CD30, EGFR, GD2, HER1, HER2, MUC1, PSMA, or RQR8.

In some embodiments, a safety switch may be a transgene encoding a product with cell killing capabilities when activated by a drug or prodrug, for example, by turning a non-toxic prodrug to a toxic metabolite inside the cell. In these embodiments, cell killing is activated by contacting a cell comprising the vector with the drug or prodrug. In some cases, a safety switch is HSVtk, which converts ganciclovir (GCV) to GCV-triphosphate, thereby interfering with DNA synthesis and killing dividing cells. In some cases, a safety switch is CyD or a variant thereof, which converts the antifungal drug 5-fluorocytosine (5-FC) to cytotoxic 5-fluorouracil (5-FU) by catalyzing the hydrolytic deamination of cytosine into uracil. 5-FU is further converted to potent anti-metabolites (5-FdUMP, 5-FdUTP, 5-FUTP) by cellular enzymes. These compounds inhibit thymidylate synthase and the production of RNA and DNA, resulting in cell death. In some cases, a safety switch is NTR or a variant thereof, which can act on the prodrug CB1954 via reduction of the nitro groups to reactive N-hydroxylamine intermediates that are toxic in proliferating and nonproliferating cells. In some cases, a safety switch is PNP or a variant thereof, which can turn prodrug 6-methylpurine deoxyriboside or fludarabine into toxic metabolites to both proliferating and nonproliferating cells. In some cases, a safety switch is horseradish peroxidase or a variant thereof, which can catalyze indole-3-acetic acid (IAA) to a potent cytotoxin and thus achieve cell killing.

In some embodiments, a safety switch may be an iCasp9. Caspase 9 is a component of the intrinsic mitochondrial apoptotic pathway which, under physiological conditions, is activated by the release of cytochrome C from damaged mitochondria. Activated caspase 9 then activates caspase 3, which triggers terminal effector molecules leading to apoptosis. iCasp9 may be generated by fusing a truncated caspase 9 (without its physiological dimerization domain or caspase activation domain) to a FK506 binding protein (FKBP), FKBP12-F36V, via a peptide linker. iCasp9 has low dimer-independent basal activity and can be stably expressed in host cells (e.g., human T cells) without impairing their phenotype, function, or antigen specificity. However, in the presence of chemical inducer of dimerization (CID), such as rimiducid (AP1903), AP20187, and rapamycin, iCasp9 can undergo inducible dimerization and activate the downstream caspase molecules, resulting in apoptosis of cells expressing the iCasp9. See, e.g., PCT Application Publication No. WO2011/146862; Stasi et al., N. Engl. J. Med. 365; 18 (2011); Tey et al., Biol. Blood Marrow Transplant 13:913-924 (2007). In particular, the rapamycin-inducible caspase 9 variant is called rapaCasp9. See Stavrou et al., Mol. Ther. 26(5):1266-1276 (2018). Thus, iCasp9 can be used as a safety switch in the present polycistronic vector to achieve controlled killing of the host cells.

In some embodiments, a safety switch may be a membrane-expressed protein which allows for cell depletion after administration of a specific antibody to that protein. Safety switches of this category may include, for example, CCR4, CD16, CD19, CD20, CD30, EGFR, GD2, HER1, HER2, MUC1, PSMA, or RQR8. These proteins may have surface epitopes that can be targeted by specific antibodies.

In some embodiments, a safety switch comprises CCR4, which can be recognized by an anti-CCR4 antibody. Non-limiting examples of suitable anti-CCR4 antibodies include mogamulizumab and biosimilars thereof.

In some embodiments, a safety switch comprises CD16 or CD30, which can be recognized by an anti-CD16 or anti-CD30 antibody. Non-limiting examples of such anti-CD16 or anti-CD30 antibody include AFM13 and biosimilars thereof.

In some embodiments, a safety switch comprises CD19, which can be recognized by an anti-CD19 antibody. Non-limiting examples of such anti-CD19 antibody include MOR208 and biosimilars thereof.

In some embodiments, a safety switch comprises CD20, which can be recognized by an anti-CD20 antibody. Non-limiting examples of such anti-CD20 antibody include obinutuzumab, ublituximab, ocaratuzumab, rituximab, rituximab-RLIb, and biosimilars thereof. Cells that express the safety switch are thus CD20-positive and can be targeted for killing through administration of an anti-CD20 antibody as described.

In some embodiments, a safety switch comprises EGFR, which can be recognized by an anti-EGFR antibody. Non-limiting examples of such anti-EGFR antibody include tomuzotuximab, R05083945 (GA201), cetuximab, and biosimilars thereof.

In some embodiments, a safety switch comprises GD2, which can be recognized by an anti-GD2 antibody. Non-limiting examples of such anti-GD2 antibody include Hul4.18K322A, Hul4.18-IL2, Hu3F8, dinituximab, c.60C3-RLIc, and biosimilars thereof.

In some embodiments, a safety switch comprises HER1, which can be recognized by an anti-HER1 antibody. Non-limiting examples of such anti-HER1 antibody include cetuximab and biosimilars thereof.

In some embodiments, a safety switch comprises HER2, which can be recognized by an anti-HER2 antibody. Non-limiting examples of such anti-HER2 antibody include margetuximab, trastuzumab, TrasGEX, and biosimilars thereof.

In some embodiments, a safety switch comprises MUC1, which can be recognized by an anti-MUC1 antibody. Non-limiting examples of such anti-MUC1 antibody include gatipotuzumab and biosimilars thereof.

In some embodiments, a safety switch comprises PSMA, which can be recognized by an anti-PSMA antibody. Non-limiting examples of such anti-PSMA antibody include KM2812 and biosimilars thereof.

In some embodiments, a safety switch comprises RQR8, which can be recognized by an anti-RQR8 antibody. Non-limiting examples of such anti-RQR8 antibody include rituximab and biosimilars thereof.

In some embodiments, a safety switch comprises HSVtk and a membrane-expressed protein, for example, CCR4, CD16, CD19, CD20, CD30, EGFR, GD2, HER1, HER2, MUC1, PSMA, and RQR8.

In some embodiments, wherein the modified immune evasive cell is inserted with a transgene encoding CD47 or wherein the vector comprises a CD47 coding sequence, a CD47-SIRPα blockade agent can be used as a safety switch.

Without wishing to be bound by theory, it is believed that the modifications of the engineered cells “cloak” them from the recipient immune system's effector cells that are responsible for the clearance of infected, malignant or non-self cells. “Cloaking” of a cell from the immune system allows for existence and persistence of specific cells, e.g., allogeneic cells within the body. In some instances, engineered cells described herein may no longer be therapeutically effective or may induce undesired adverse effects in the recipient. Non-limiting examples of an adverse event include hyperproliferation, transformation, tumor formation, cytokine release syndrome, GVHD, immune effector cell-associated neurotoxicity syndrome (ICANS), inflammation, infection, nausea, vomiting, bleeding, interstitial pneumonitis, respiratory disease, jaundice, weight loss, diarrhea, loss of appetite, cramps, abdominal pain, hepatic veno-occlusive disease (VOD), graft failure, organ damage, infertility, hormonal changes, abnormal growth formation, cataracts, and post-transplant lymphoproliferative disorder (PTLD), and the like. Controlled removal of the engineered cells from the body is crucial for patient safety and can be achieved by uncloaking the cells from the immune system. Uncloaking serves as a safety switch and can be achieved through the downregulation of the immunosuppressive molecules or the upregulation of immune signaling molecules. The level of expression of any of the immunosuppressive molecules described can be controlled on the protein level, mRNA level, or DNA level in the cells. Similarly, the level of expression of any of the immune signaling molecules described can be controlled on the protein level, mRNA level, or DNA level in the cells. In an example of uncloaking Hypo-Immune cells Through Genetic, Post-Transcriptional, and Post-Translational Regulation, hypoimmunity is achieved through the overexpression of hypoimmune molecules such as CD47, complement inhibitors accompanied with the repression or genetic disruption of the HLA-I and HLA-II loci. These modifications cloak the cell from the immune system's effector cells that are responsible for the clearance of infected, malignant or non-self cells, such as T-cells, B-cells, NK cells and macrophages. Cloaking of a cell from the immune system allows for existence and persistence of allogeneic cells within the body. Removal of the engineered cells from the body is crucial for patient safety and can be achieved by uncloaking the cells from the immune system. Uncloaking serves as a safety switch and can be achieved through the downregulation of the hypoimmune molecules (for example CD47, A20/TNFAIP3, B2M-HLA-E, CD16, CD16 Fc receptor, CD24, CD27, CD35, CD39, CD46, CD47, CD52, CD55, CD59, CD64, CD200, CCL21, CCL22, CTLA4-Ig, C1 inhibitor, CR1, DUX4, FASL, HLA-C, HLA-E, HLA-E heavy chain, HLA-F, HLA-G, H2-M3, IDO1, IL-10, IL15-RF, IL-35, IL-39, MANF, Mfge8, PD-L1, and Serpinb9) or the upregulation of immune signaling molecules (for example B2M, MIC-A/B, HLA-A, HLA-B, HLA-C, HLA-D, HLA-E, RFXANK, CTLA-4, PD-1, CIITA, HLA-DP, HLA-DM, HLA-DOA, HLA-DOB, HLA-DQ, HLA-DR, and ligands of NKG2D (e.g., MICA, MICB, RAETIE/ULBP4, RAETIG/ULBP5, RAETIH/ULBP2, RAET1/ULBP1, RAETIL/ULBP6, or RAETIN/ULBP3). Either of these activities, or a combination of the both, will avail the cell to native effector cells, resulting in clearance of the allogeneic cell.

In some embodiments, upon contacting the cells with a CD47-SIRPα blockade agent, the cells are recognized by the recipient's immune system. In some embodiments, the engineered cells express the immunosuppressive factor CD47 such that the cells are immune evasive or have reduced immunogenicity until one or more CD47-SIRPα blockade agents are administered to the recipient. In the presence of a CD47-SIRPα blockade agent, the cells are uncloaked and are recognized by immune cells to be targeted by cell death or clearance.

In some embodiments, administration of a CD47-SIRPα blockade agent to the recipient facilitates phagocytosis, cell clearance and/or cell death of these cells and derivatives thereof (e.g., progeny cells). In some aspects, the CD47-SIRPα blockade agent is an agent that neutralizes, blocks, antagonizes, or interferes with the cell surface expression of CD47, SIRPα, or both. In some embodiments, the CD47-SIRPα blockade agent inhibits or blocks the interaction of CD47, SIRPα or both. Such CD47-SIRPα blockade agents are useful as safety switches to modulate the activity of administered or engrafted cells, thereby improving the safety of these cell-based therapies.

### 1. CD47-SIRPα Blockade Agents

In some embodiments, a patient is treated with a therapeutic agent that inhibits or blocks the interaction of CD47 and SIRPα. In some embodiments, a CD47-SIRPα blockade agent (e.g., a CD47-SIRPα blocking, inhibiting, reducing, antagonizing, neutralizing, or interfering agent) comprises an agent selected from a group that includes an antibody or fragment thereof that binds CD47, a bispecific antibody that binds CD47, an immunocytokine fusion protein that bind CD47, a CD47 containing fusion protein, an antibody or fragment thereof that binds SIRPα, a bispecific antibody that binds SIRPα, an immunocytokine fusion protein that bind SIRPα, an SIRPα containing fusion protein, and a combination thereof.

In some aspects, the CD47-SIRPα blockade agent reduces in a patient the number of cells exogenously expressing CD47 polypeptides, including, but not limited to, cells that also exogenously express one or more chimeric antigen receptors. In some embodiments, the CD47-SIRPα blockade agent decreases the number of CD47-expressing immune evasive cells in the patient, independent of the level of CAR expression by such cells. In some instances, the level of CAR expression by the cells is less (e.g., 1%, 5%, 10%, 15%, 20%, 25%, 30%, 40%, 50%, 60%, 70%, 80%, 90%, 95%, or 99% less) than the level by a control CAR-T cell, such as, but not limited to, a tisagenlecleucel biosimilar, tisagenlecleucel surrogate and the like. In certain instances, the level of CAR expression by the cells is more (e.g., 1%, 5%, 10%, 15%, 20%, 25%, 30%, 40%, 50%, 60%, 70%, 80%, 90%, 95%, 100%, 150%, 200%, 300%, or a higher percentage more) than the level by a control CAR-T cell, such as, but not limited to, a tisagenlecleucel biosimilar, tisagenlecleucel surrogate and the like.

### A. CD47-Binding Blockade Agents

In some embodiments, a CD47-SIRPα blockade agent is an agent that binds CD47. An agent can be a CD47 blocking, neutralizing, antagonizing or interfering agent. In some embodiments, a CD47-SIRPα blockade agent is selected from a group that includes an antibody or fragment thereof that binds CD47, a bispecific antibody that binds CD47, and an immunocytokine fusion protein that binds CD47.

Useful antibodies or fragments thereof that bind CD47 can be selected from a group that includes magrolimab ((Hu5F9-G4)) (Forty Seven, Inc.; Gilead Sciences, Inc.), urabrelimab, CC-90002 (Celgene; Bristol-Myers Squibb), IBI-188 (letaplimab, Innovent Biologics), IBI-322 (Innovent Biologics), TG-1801 (TG Therapeutics; also known as NI-1701, Novimmune SA), ALX148 (ALX Oncology), TJ011133 (also known as TJC4, I-Mab Biopharma), FA3M3, ZL-1201 (Zai Lab Co., Ltd), AK117 (Akesbio Australia Pty, Ltd.), AO-176 (Arch Oncology), SRF231 (Surface Oncology), GenSci-059 (GeneScience), C47B157 (Janssen Research and Development), C47B161 (Janssen Research and Development), C47B167 (Janssen Research and Development), C47B222 (Janssen Research and Development), C47B227 (Janssen Research and Development), Vx-1004 (Corvus Pharmaceuticals), HMBDOO4 (Hummingbird Bioscience Pte Ltd), SHR-1603 (Hengrui), AMMS4-G4 (Beijing Institute of Biotechnology), RTX-CD47 (University of Groningen), STI-6643 (Sorrento), and IMC-002 (Samsung Biologics; ImmuneOncia Therapeutics). In some embodiments, an antibody or fragment thereof does not compete for CD47 binding with an antibody selected from a group that includes magrolimab, urabrelimab, CC-90002, IBI-188, IBI-322, TG-1801 (NI-1701), ALX148, TJ011133, FA3M3, ZL1201, AK117, AO-176, SRF231, GenSci-059, C47B157, C47B161, C47B167, C47B222, C47B227, Vx-1004, HMBDOO4, SHR-1603, AMMS4-G4, RTX-CD47, and IMC-002. In some embodiments, an antibody or fragment thereof competes for CD47 binding with an antibody selected from magrolimab, urabrelimab, CC-90002, IBI-188, IBI-322, TG-1801 (NI-1701), ALX148, TJ011133, FA3M3, ZL1201, AK117, AO-176, SRF231, GenSci-059, C47B157, C47B161, C47B167, C47B222, C47B227, Vx-1004, HMBD004, SHR-1603, AMMS4-G4, RTX-CD47, and IMC-002. In some embodiments, the antibody or fragment thereof that binds CD47 is selected from a group that includes a single-chain Fv fragment (scFv) against CD47, a Fab against CD47, a VHH nanobody against CD47, a DARPin against CD47, and variants thereof. In some embodiments, the scFv against CD47, a Fab against CD47, and variants thereof are based on the antigen binding domains of any of the antibodies selected from a group that includes magrolimab, urabrelimab, CC-90002, IBI-188, IBI-322, TG-1801 (NI-1701), ALX148, TJ011133, FA3M3, ZL1201, AK117, AO-176, SRF231, GenSci-059, C47B157, C47B161, C47B167, C47B222, C47B227, Vx-1004, HMBD004, SHR-1603, AMMS4-G4, RTX-CD47, and IMC-002.

Useful bispecific antibodies that bind CD47 comprise a first antigen binding domain that binds CD47 and a second antigen binding domain that binds an antigen selected from a group that includes CD19, CD20, CD22, CD24, CD25, CD30, CD33, CD38, CD44, CD52, CD56, CD70, CD96, CD97, CD99, CD123, CD279 (PD-1), EGFR, HER2, CD117, c-Met, PTHR2, HAVCR2 (TIM3), and an antigen expressed on a cancer cell.

In some embodiments, a CD47-SIRPα blockade agent is an immunocytokine fusion protein comprising a cytokine and either an antigen binding domain, antibody, or fragment thereof that binds CD47.

Detailed descriptions of exemplary CD47 binding molecules (e.g., antigen binding domains, antibodies, nanobodies, diabodies, antibody mimetic proteins (e.g., DARPins), and fragments thereof that recognize or bind CD47) including sequences of the heavy chain, light chain, VH region, VL region, CDRs, and framework regions can be found, for example, in WO2009091601; WO2011143624; WO2013119714; WO201414947; WO2014149477; WO2015138600; WO2016033201; WO2017049251; Pietsch et al., Blood Cancer J, 2017, 7(2), e536; van Brommel et al., 2018, 7(2), e1386361; Yu et al., Biochimie, 2018, 151, 54-66; and Andrechak et al., Phil Trans R Soc, 2019, 374, 20180217; the disclosures such as the sequence listings, specifications, and figures are herein incorporated in their entirety.

b. SIRPα-Binding Blockade Agents

In some embodiments, a CD47-SIRPα blockade agent administered to the recipient subject is an agent that binds SIRPα. An agent can be an SIRPα blocking, neutralizing, antagonizing or inactivating agent. In some embodiments, a CD47-SIRPα blockade agent is selected from a group that includes, but is not limited to, an antibody or fragment thereof that binds SIRPα, a bispecific antibody that binds SIRPα, and an immunocytokine fusion protein that bind SIRPα.

Useful antibodies or fragments thereof that bind SIRPα can be selected from a group that includes, but is not limited to, ADU-1805 (Aduro Biotech Holdings), OSE-172 (OSE Immunotherapeutics; also known as BI 765063 by Boehringer Ingelheim), CC-95251 (Celgene; Bristol-Myers Squibb), KWAR23 (Leland Stanford Junior University), and P362 (Leland Stanford Junior University). In some embodiments, an antibody or fragment thereof does not compete for SIRPα binding with an antibody selected from a group that includes ADU-1805, CC-95251, OSE-172 (BI 765063), KWAR23, and P362. In some embodiments, an antibody or fragment thereof competes for SIRPα binding with an antibody selected from a group that includes ADU-1805, CC-95251, OSE-172 (BI 765063), KWAR23, and P362.

In some embodiments, an antibody or fragment thereof that binds SIRPα is selected from a group that includes a single-chain Fv fragment (scFv) against SIRPα, a Fab against SIRPα, a VHH nanobody against SIRPα, a DARPin against SIRPα, and variants thereof. In some embodiments, an scFv against SIRPα, a Fab against SIRPα, and variants thereof are based on the antigen binding domains of any of the antibodies selected from a group that includes ADU-1805, CC-95251, OSE-172 (BI 765063), KWAR23, and P362.

In some embodiments, a bispecific antibody that binds SIRPα and an antigen binding domain that binds an antigen selected from a group that includes CD19, CD20, CD22, CD24, CD25, CD30, CD33, CD38, CD44, CD52, CD56, CD70, CD96, CD97, CD99, CD123, CD279 (PD-1), EGFR, HER2, CD117, C-Met, PTHR2, HAVCR2 (TIM3), and an antigen expressed on a cancer cell. In some instances, a bispecific antibody binds SIRPα and a tumor associated antigen. In some instances, the bispecific antibody binds SIRPα and an antigen expressed on the surface of an immune cell.

In some embodiments, a CD47-SIRPα blockade agent is an immunocytokine fusion protein comprises a cytokine and either an antigen binding domain, antibody, or fragment thereof that binds SIRPα.

Detailed descriptions of exemplary SIRPα binding molecules (e.g., antigen binding domains, antibodies, nanobodies, diabodies, antibody mimetic proteins (e.g., DARPins), and fragments thereof that recognize or bind SIRPα) including sequences of the heavy chain, light chain, VH region, VL region, CDRs, and framework regions can be found, for example, in WO2019226973; WO2018190719; WO2018057669; WO2017178653; WO2016205042; WO2016033201; WO2016022971; WO2015138600; and WO2013109752; the disclosures including the sequence listings, specifications, and figures are herein incorporated in their entirety.

C. CD47- and/or SIRP-Containing Fusion Proteins

As disclosed herein, a CD47-SIRPα blockade agent can comprise a CD47-containing fusion protein that binds SIRPα. In some embodiments, such CD47-containing fusion protein that binds SIRPα is an agent administered to a recipient subject. In some embodiments, a CD47-containing fusion protein comprises a CD47 extracellular domain or variants thereof that bind SIRPα. In some embodiments, the fusion protein comprises an Fc region. Detailed descriptions of exemplary CD47 fusion proteins including sequences can be found, for example, in US20100239579, the disclosure is herein incorporated in its entirety including the sequence listing, specification, and figure.

In some embodiments, a CD47-SIRPα blockade agent can comprise an SIRPα-containing fusion protein that binds CD47. The sequence of SIRPα is set forth in SEQ ID NO:13 (UniProt P78324). Generally, SIRPα-containing fusion proteins comprise a domain of SIRPα including any one of (a) the immunoglobulin-like domain of human SIRPα (e.g., the membrane distal (D1) loop containing an IgV domain of SIRP, (b) the first membrane proximal loop containing an IgC domain, and (c) the second membrane proximal loop containing an IgC domain). In some instances, the SIRPα domain binds CD47. In some embodiments, the SIRPα-containing fusion protein comprises an SIRPα extracellular domain or variants thereof that bind CD47. In some embodiments, the fusion protein comprises an Fc region, including but not limited to a human IgG1 Fc region (e.g., UniProtKB/Swiss-Prot P01857, SEQ ID NO:14) or IgG4 Fc region (e.g., UniProt P01861, SEQ ID NO:15; GenBank CAC20457.1, SEQ ID NO:16). Optionally, the Fc region may comprise one or more substitutions. In some embodiments, the SIRPα-containing fusion proteins are selected from a group that includes TTI-621 (Trillium Therapeutics), TTI-622 (Trillium Therapeutics), and ALX148 (ALX Oncology). TTI-621 (SEQ ID NO:17) is a fusion protein made up of the N-terminal V domain of human SIRPα fused to a human IgG1 Fc region (Petrova et al. Clin Cancer Res 23(4):1068-1079 (2017)), while TTI-622 (SEQ ID NO:18) is a fusion protein made up of the N-terminal V domain of human SIRPα fused to a human IgG4 Fc region with a single substitution.

TABLE 2
Exemplary sequences of SIRPα, IgG1/IgG4, and CD47 fusion proteins

| SEQ ID NO: | Sequence | Description |
|---|---|---|
| 13 | MEPAGPAPGRLGPLLCLLLAASCAWSGVAGEEELQVI | SIRPα (UniProt P78324) |
| | QPDKSVSVAAGESAILHCTVTSLIPVGPIQWFRGAGPAR | |
| | ELIYNQKEGHFPRVTTVSESTKRENMDFSISISNITPADA | |
| | GTYYCVKFRKGSPDTEFKSGAGTELSVRAKPSAPVVSG | |
| | PAARATPQHTVSFTCESHGFSPRDITLKWFKNGNELSD | |
| | FQTNVDPVGESVSYSIHSTAKVVLTREDVHSQVICEVA | |
| | HVTLQGDPLRGTANLSETIRVPPTLEVTQQPVRAENQV | |
| | NVTCQVRKFYPQRLQLTWLENGNVSRTETASTVTENK | |
| | DGTYNWMSWLLVNVSAHRDDVKLTCQVEHDGQPAV | |
| | SKSHDLKVSAHPKEQGSNTAAENTGSNERNIYIVVGVV | |
| | CTLLVALLMAALYLVRIRQKKAQGSTSSTRLHEPEKNA | |
| | REITQVQSLDTNDITYADLNLPKGKKPAPQAAEPNNHT | |
| | EYASIQTSPQPASEDTLTYADLDMVHLNRTPKQPAPKP | |
| | EPSFSEYASVQVPRK | |
| 14 | ASTKGPSVFPLAPSSKSTSGGTAALGCLVKDYFPEPVT | Human IgG1 |
| | VSWNSGALTSGVHTFPAVLQSSGLYSLSSVVTVPSSSL | (UniProtKB/Swiss-Prot |
| | GTQTYICNVNHKPSNTKVDKKVEPKSCDKTHTCPPCPA | P01857) |
| | PELLGGPSVFLFPPKPKDTLMISRTPEVTCVVVDVSHED | |
| | PEVKFNWYVDGVEVHNAKTKPREEQYNSTYRVVSVL | |
| | TVLHQDWLNGKEYKCKVSNKALPAPIEKTISKAKGQP | |
| | REPQVYTLPPSRDELTKNQVSLTCLVKGFYPSDIAVEW | |
| | ESNGQPENNYKTTPPVLDSDGSFFLYSKLTVDKSRWQ | |
| | QGNVFSCSVMHEALHNHYTQKSLSLSPGK | |
| 15 | ASTKGPSVFPLAPCSRSTSESTAALGCLVKDYFPEPVTV | Human IgG4 (UniProt |
| | SWNSGALTSGVHTFPAVLQSSGLYSLSSVVTVPSSSLG | P01861) |
| | TKTYTCNVDHKPSNTKVDKRVESKYGPPCPSCPAPEFL | |
| | GGPSVFLFPPKPKDTLMISRTPEVTCVVVDVSQEDPEV | |
| | QFNWYVDGVEVHNAKTKPREEQFNSTYRVVSVLTVL | |
| | HQDWLNGKEYKCKVSNKGLPSSIEKTISKAKGQPREPQ | |
| | VYTLPPSQEEMTKNQVSLTCLVKGFYPSDIAVEWESNG | |
| | QPENNYKTTPPVLDSDGSFFLYSRLTVDKSRWQEGNVF | |
| | SCSVMHEALHNHYTQKSLSLSLGK | |
| 16 | ASFKGPSVFPLVPCSRSTSESTAALGCLVKDYFPEPVTV | Human IgG4 (GenBank |
| | SWNSCALTSGVHTFPAVLQSSGLYSLSSVVTVPSSSLGT | CAC20457.1) |
| | KTYTCNVDHKPSNTKVDKRVESKYGPPCPSCPAPEFLG | |
| | GPSVFLFPPKPKDTLMISRTPEVTCVVVDVSQEDPEVQF | |
| | NWYVDGVEVHNAKTKPREEQFNSTYRVVRVLTVLHQ | |
| | DWLNGKEYKCKVSNKGLPSSIEKTISKAKGQPREPQVY | |
| | TLPPSQEEMTKNQVSLTCLVKGFYPSDIAVEWESNGQP | |
| | EDNYKTTPPVLDSDGSFFLYSRLTVDKSRWQEGNVFSC | |
| | SVMHEALHNHYTQKSLSLSPGK | |
| 17 | EEELQVIQPDKSVSVAAGESAILHCTVTSLIPVGPIQWF | TTI-621 |
| | RGAGPARELIYNQKEGHFPRVTTVSESTKRENMDFSISI | |
| | SNITPADAGTYYCVKFRKGSPDTEFKSGAGTELSVRAK | |
| | PSDKTHTCPPCPAPELLGGPSVFLFPPKPKDTLMISRTPE | |
| | VTCVVVDVSHEDPEVKFNWYVDGVEVHNAKTKPREE | |
| | QYNSTYRVVSVLTVLHQDWLNGKEYKCKVSNKALPA | |
| | PIEKTISKAKGQPREPQVYTLPPSRDELTKNQVSLTCLV | |
| | KGFYPSDIAVEWESNGQPENNYKTTPPVLDSDGSFFLY | |
| | SKLTVDKSRWQQGNVFSCSVMHEALHNHYTQKSLSLS | |
| | PGK | |
| 18 | EEELQVIQPDKSVSVAAGESAILHCTVTSLIPVGPIQWF | TTI-622 |
| | RGAGPARELIYNQKEGHFPRVTTVSESTKRENMDFSISI | |
| | SNITPADAGTYYCVKFRKGSPDTEFKSGAGTELSVRAK | |
| | PSESKYGPPCPPCPAPEFLGGPSVFLFPPKPKDTLMISRT | |
| | PEVTCVVVDVSQEDPEVQFNWYVDGVEVHNAKTKPR | |
| | EEQFNSTYRVVSVLTVLHQDWLNGKEYKCKVSNKGLP | |
| | SSIEKTISKAKGQPREPQVYTLPPSQEEMTKNQVSLTCL | |
| | VKGFYPSDIAVEWESNGQPENNYKTTPPVLDSDGSFFL | |
| | YSRLTVDKSRWQEGNVFSCSVMHEALHNHYTQKSLSL | |
| | SLGK | |

TTI-621, TTI-622, and other related fusion proteins are disclosed in PCT Publ. No. WO14/94122, the contents of which are hereby incorporated by reference herein with regard to said proteins. AL148 is a fusion protein made up of the N-terminal D1 domain of SIRPα fused to a modified human IgG1 Fc domain (Kauder et al. PLoS One (13(8):e0201832 (2018)). Detailed descriptions of exemplary SIRPα fusion proteins including sequences can be found, for example, in PCT Publ. Nos. WO14/94122; WO16/23040; WO17/27422; WO17/177333; and WO18/176132, the disclosures of which are hereby incorporated herein in their entirety, including the sequence listings, specifications, and figures.

SIRPα-containing fusion proteins, including TTI-621, are being developed for the treatment of cancer, such as hematologic malignancies, alone or in combination with other cancer therapy drugs. A phase 1 trial evaluating dosage and safety (NCT02663518) of intravenous TTI-621 administration in patients with relapsed/refractory hematologic malignancies and selected solid tumors found that TTI-621 was well tolerated and demonstrated activity both as a monotherapy and in combination with other cancer treatment agents (Ansell et al. Clin Cancer Res 27(8):2190-2199 (2021)). In the initial escalation phase, subjects received TTI-621 at dosages of 0.05, 0.1, 0.3, 1, 3, and 10 mg/kg to evaluate safety and maximum tolerated dose (MTD). In the expansion phase, subjects received the MTD of 0.2 mg/kg as a monotherapy or 0.1 mg/kg in combination with rituximab or nivolumab.

### E. Chimeric Antigen Receptors

Provided herein are hypoimmunogenic cells comprising a chimeric antigen receptor (CAR). In some embodiments, the CAR binds to CD22. In some embodiments, the CAR binds to CD19 and CD22. In some embodiments, the CAR is selected from the group consisting of a first generation CAR, a second generation CAR, a third generation CAR, and a fourth generation CAR. In some embodiments, the CAR includes a single binding domain that binds to a single target antigen. In some embodiments, the CAR includes a single binding domain that binds to more than one target antigen, e.g., 2, 3, or more target antigens. In some embodiments, the CAR includes two binding domains such that each binding domain binds to a different target antigens. In some embodiments, the CAR includes two binding domains such that each binding domain binds to the same target antigen. Detailed descriptions of exemplary CARs including CD19-specific, CD22-specific and CD19/CD22-bispecific CARs can be found in WO2012/079000, WO2016/149578 and WO2020/014482, the disclosures including the sequence listings and figures are incorporated herein by reference in their entirety.

In some embodiments, the CD19 specific CAR includes an anti-CD19 single-chain antibody fragment (scFv), a transmembrane domain such as one derived from human CD8a, a 4-1BB (CD137) co-stimulatory signaling domain, and a CD3ζ signaling domain. In some embodiments, the CD22 specific CAR includes an anti-CD22 scFv, a transmembrane domain such as one derived from human CD8a, a 4-1BB (CD137) co-stimulatory signaling domain, and a CD3ζ signaling domain. In some embodiments, the CD19/CD22-bispecific CAR includes an anti-CD19 scFv, an anti-CD22 scFv, a transmembrane domain such as one derived from human CD8a, a 4-1BB (CD137) co-stimulatory signaling domain, and a CD3ζ signaling domain.

In some embodiments, the CAR comprises a commercial CAR construct carried by a T cell. Non-limiting examples of commercial CAR-T cell based therapies include brexucabtagene autoleucel (TECARTUS®), axicabtagene ciloleucel (YESCARTA®), idecabtagene vicleucel (ABECMA®), lisocabtagene maraleucel (BREYANZI®), tisagenlecleucel (KYMRIAH®), Descartes-08 and Descartes-11 from Cartesian Therapeutics, CTL110 from Novartis, P-BMCA-101 from Poseida Therapeutics, AUTO4 from Autolus Limited, UCARTCS from Cellectis, PBCAR19B and PBCAR269A from Precision Biosciences, FT819 from Fate Therapeutics, and CYAD-211 from Clyad Oncology.

In some embodiments, a hypoimmunogenic cell described herein comprises a polynucleotide encoding a chimeric antigen receptor (CAR) comprising an antigen binding domain. In some embodiments, a hypoimmunogenic cell described herein comprises a chimeric antigen receptor (CAR) comprising an antigen binding domain. In some embodiments, the polynucleotide is or comprises a chimeric antigen receptor (CAR) comprising an antigen binding domain. In some embodiments, the CAR is or comprises a first generation CAR comprising an antigen binding domain, a transmembrane domain, and at least one signaling domain (e.g., one, two or three signaling domains). In some embodiments, the CAR comprises a second generation CAR comprising an antigen binding domain, a transmembrane domain, and at least two signaling domains. In some embodiments, the CAR comprises a third generation CAR comprising an antigen binding domain, a transmembrane domain, and at least three signaling domains. In some embodiments, a fourth generation CAR comprising an antigen binding domain, a transmembrane domain, three or four signaling domains, and a domain which upon successful signaling of the CAR induces expression of a cytokine gene. In some embodiments, the antigen binding domain is or comprises an antibody, an antibody fragment, an scFv or a Fab.

### 1. Antigen Binding Domain (ABD) Targets an Antigen Characteristic of a Neoplastic or Cancer Cell

In some embodiments, the antigen binding domain (ABD) targets an antigen characteristic of a neoplastic cell. In other words, the antigen binding domain targets an antigen expressed by a neoplastic or cancer cell. In some embodiments, the ABD binds a tumor associated antigen. In some embodiments, the antigen characteristic of a neoplastic cell (e.g., antigen associated with a neoplastic or cancer cell) or a tumor associated antigen is selected from a cell surface receptor, an ion channel-linked receptor, an enzyme-linked receptor, a G protein-coupled receptor, receptor tyrosine kinase, tyrosine kinase associated receptor, receptor-like tyrosine phosphatase, receptor serine/threonine kinase, receptor guanylyl cyclase, histidine kinase associated receptor, epidermal growth factor receptors (EGFR) (including ErbB1/EGFR, ErbB2/HER2, ErbB3/HER3, and ErbB4/HER4), fibroblast growth factor receptors (FGFR) (including FGF1, FGF2, FGF3, FGF4, FGF5, FGF6, FGF7, FGF18, and FGF21), vascular endothelial growth factor receptors (VEGFR) (including VEGF-A, VEGF-B, VEGF-C, VEGF-D, and PIGF), RET Receptor and the Eph Receptor Family (including EphA1, EphA2, EphA3, EphA4, EphA5, EphA6, EphA7, EphA8, EphA9, EphA10, EphB1, EphB2. EphB3, EphB4, and EphB6), CXCR1, CXCR2, CXCR3, CXCR4, CXCR6, CCR1, CCR2, CCR3, CCR4, CCR5, CCR6, CCR8, CFTR, CIC-1, CIC-2, CIC-4, CIC-5, CIC-7, CIC-Ka, CIC-Kb, Bestrophins, TMEM16A, GABA receptor, glycin receptor, ABC transporters, NAV1.1, NAV1.2, NAV1.3, NAV1.4, NAV1.5, NAV1.6, NAV1.7, NAV1.8, NAV1.9, sphingosin-1-phosphate receptor (S1P1R), NMDA channel, transmembrane protein, multispan transmembrane protein, T-cell receptor motifs, T-cell alpha chains, T-cell β chains, T-cell γ chains, T-cell δ chains, CCR7, CD3, CD4, CD5, CD7, CD8, CD11b, CD11c, CD16, CD19, CD20, CD21, CD22, CD25, CD28, CD34, CD35, CD40, CD45RA, CD45RO, CD52, CD56, CD62L, CD68, CD80, CD95, CD117, CD127, CD133, CD137 (4-1BB), CD163, F4/80, IL-4Ra, Sca-1, CTLA-4, GITR, GARP, LAP, granzyme B, LFA-1, transferrin receptor, NKp46, perforin, CD4+, Th1, Th2, Th17, Th40, Th22, Th9, Tfh, canonical Treg. FoxP3+, Tr1, Th3, Treg17, T_{RE}G; CDCP, NT5E, EpCAM, CEA, gpA33, mucins, TAG-72, carbonic anhydrase IX, PSMA, folate binding protein, gangliosides (e.g., CD2, CD3, GM2), Lewis-γ², VEGF, VEGFR 1/2/3, αVβ, α5β1, ErbB1/EGFR, ErbB1/HER2, ErB3, c-MET, IGF1R, EphA3, TRAIL-R1, TRAIL-R2, RANKL, FAP, Tenascin, PDL-1, BAFF, HDAC, ABL, FLT3, KIT, MET, RET, IL-1β, ALK, RANKL, mTOR, CTLA-4, IL-6, IL-6R, JAK3, BRAF, PTCH, Smoothened, PIGF, ANPEP, TIMP1, PLAUR, PTPRJ, LTBR, ANTXR1, folate receptor alpha (FRa), ERBB2 (Her2/neu), EphA2, IL-13Ra2, epidermal growth factor receptor (EGFR), mesothelin, TSHR, CD19, CD123, CD22, CD30, CD171, CS-1, CLL-1, CD33, EGFRvIII, GD2, GD3, BCMA, MUC16 (CA125), L1CAM, LeY, MSLN, IL13R1a, L1-CAM, Tn Ag, prostate specific membrane antigen (PSMA), ROR1, FLT3, FAP, TAG72, CD38, CD44v6, CEA, EPCAM, B7H3, KIT, interleukin-11 receptor a (IL-11Ra), PSCA, PRSS21, VEGFR2, LewisY, CD24, platelet-derived growth factor receptor-beta (PDGFR-beta), SSEA-4, CD20, MUC1, NCAM, Prostase, PAP, ELF2M, Ephrin B2, IGF-1 receptor, CAIX, LMP2, gp100, bcr-abl, tyrosinase, Fucosyl GM1, sLe, GM3, TGS5, HMWMAA, o-acetyl-GD2, folate receptor beta, TEM1/CD248, TEM7R, CLDN6, GPRC5D, CXORF61, CD97, CD179a, ALK, Polysialic acid, PLACI, GloboH, NY-BR-1, UPK2, HAVCR1, ADRB3, PANX3, GPR20, LY6K, OR51E2, TARP, WTi, NY-ESO-1, LAGE-la, MAGE-A1, legumain, HPV E6, E7, ETV6-AML, sperm protein 17, XAGE1, Tie 2, MAD-CT-1, MAD-CT-2, major histocompatibility complex class I-related gene protein (MR1), urokinase-type plasminogen activator receptor (uPAR), Fos-related antigen 1, p53, p53 mutant, prostein, survivin, telomerase, PCTA-1/Galectin 8, MelanA/MART1, Ras mutant, hTERT, sarcoma translocation breakpoints, ML-IAP, ERG (TMPRSS2 ETS fusion gene), NA17, PAX3, androgen receptor, cyclin B1, MYCN, RhoC, TRP-2, CYPIB I, BORIS, SART3, PAX5, OY-TES1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, IGLL1, a neoantigen, CD133, CD15, CD184, CD24, CD56, CD26, CD29, CD44, HLA-A, HLA-B, HLA-C, (HLA-A, B, C) CD49f, CD151 CD340, CD200, tkrA, trkB, or trkC, or an antigenic fragment or antigenic portion thereof.

2. ABD targets an antigen characteristic of a T cell

In some embodiments, the antigen binding domain targets an antigen characteristic of a T cell. In some embodiments, the ABD binds an antigen associated with a T cell. In some instances, such an antigen is expressed by a T cell or is located on the surface of a T cell. In some embodiments, the antigen characteristic of a T cell or the T cell associated antigen is selected from a cell surface receptor, a membrane transport protein (e.g., an active or passive transport protein such as, for example, an ion channel protein, a pore-forming protein, etc.), a transmembrane receptor, a membrane enzyme, and/or a cell adhesion protein characteristic of a T cell. In some embodiments, an antigen characteristic of a T cell may be a G protein-coupled receptor, receptor tyrosine kinase, tyrosine kinase associated receptor, receptor-like tyrosine phosphatase, receptor serine/threonine kinase, receptor guanylyl cyclase, histidine kinase associated receptor, AKT1; AKT2; AKT3; ATF2; BCL10; CALM1; CD3D (CD35); CD3E (CD3ε); CD3G (CD3γ); CD4; CD8; CD28; CD45; CD80 (B7-1); CD86 (B7-2); CD247 (CD3ζ); CTLA-4 (CD152); ELK1; ERK1 (MAPK3); ERK2; FOS; FYN; GRAP2 (GADS); GRB2; HLA-DRA; HLA-DRB1; HLA-DRB3; HLA-DRB4; HLA-DRB5; HRAS; IKBKA (CHUK); IKBKB; IKBKE; IKBKG (NEMO); IL2; ITPR1; ITK; JUN; KRAS2; LAT; LCK; MAP2K1 (MEK1); MAP2K2 (MEK2); MAP2K3 (MKK3); MAP2K4 (MKK4); MAP2K6 (MKK6); MAP2K7 (MKK7); MAP3K1 (MEKK1); MAP3K3; MAP3K4; MAP3K5; MAP3K8; MAP3K14 (NIK); MAPK8 (JNK1); MAPK9 (JNK2); MAPK10 (JNK3); MAPK11 (p38β); MAPK12 (p38γ); MAPK13 (p38δ); MAPK14 (p38a); NCK; NFAT1; NFAT2; NFKB1; NFKB2; NFKB1A; NRAS; PAK1; PAK2; PAK3; PAK4; PIK3C2B; PIK3C3 (VPS34); PIK3CA; PIK3CB; PIK3CD; PIK3R1; PKCA; PKCB; PKCM; PKCQ; PLCY1; PRF1 (Perforin); PTEN; RAC1; RAF1; RELA; SDF1; SHP2; SLP76; SOS; SRC; TBK1; TCRA; TEC; TRAF6; VAV1; VAV2; or ZAP70.

In some embodiments, an antigen binding domain of a CAR binds to a ligand expressed on B cells, plasma cells, or plasmablasts. In some embodiments, an antigen binding domain of a CAR binds to CD10, CD19, CD20, CD22, CD24, CD27, CD38, CD45R, CD138, CD319, BCMA, CD28, TNF, interferon receptors, GM-CSF, ZAP-70, LFA-1, CD3 gamma, CD5 or CD2. See, e.g., US 2003/0077249; WO 2017/058753; WO 2017/058850, the contents of which are herein incorporated by reference.

### 3. ABD Binds to a Cell Surface Antigen of a Cell

In some embodiments, an antigen binding domain binds to a cell surface antigen of a cell. In some embodiments, a cell surface antigen is characteristic of (e.g., expressed by) a particular or specific cell type. In some embodiments, a cell surface antigen is characteristic of more than one type of cell.

In some embodiments, a CAR antigen binding domain binds a cell surface antigen characteristic of a T cell, such as a cell surface antigen on a T cell. In some embodiments, an antigen characteristic of a T cell may be a cell surface receptor, a membrane transport protein (e.g., an active or passive transport protein such as, for example, an ion channel protein, a pore-forming protein, etc.), a transmembrane receptor, a membrane enzyme, and/or a cell adhesion protein characteristic of a T cell. In some embodiments, an antigen characteristic of a T cell may be a G protein-coupled receptor, receptor tyrosine kinase, tyrosine kinase associated receptor, receptor-like tyrosine phosphatase, receptor serine/threonine kinase, receptor guanylyl cyclase, or histidine kinase associated receptor.

In some embodiments, an antigen binding domain of a CAR binds a T cell receptor. In some embodiments, a T cell receptor may be AKT1; AKT2; AKT3; ATF2; BCL10; CALM1; CD3D (CD35); CD3E (CD3ε); CD3G (CD3γ); CD4; CD8; CD28; CD45; CD80 (B7-1); CD86 (B7-2); CD247 (CD3ζ); CTLA-4 (CD152); ELK1; ERK1 (MAPK3); ERK2; FOS; FYN; GRAP2 (GADS); GRB2; HLA-DRA; HLA-DRB1; HLA-DRB3; HLA-DRB4; HLA-DRB5; HRAS; IKBKA (CHUK); IKBKB; IKBKE; IKBKG (NEMO); IL2; ITPR1; ITK; JUN; KRAS2; LAT; LCK; MAP2K1 (MEK1); MAP2K2 (MEK2); MAP2K3 (MKK3); MAP2K4 (MKK4); MAP2K6 (MKK6); MAP2K7 (MKK7); MAP3K1 (MEKK1); MAP3K3; MAP3K4; MAP3K5; MAP3K8; MAP3K14 (NIK); MAPK8 (JNK1); MAPK9 (JNK2); MAPK10 (JNK3); MAPK11 (p38β); MAPK12 (p38γ); MAPK13 (p38δ); MAPK14 (p38α); NCK; NFAT1; NFAT2; NFKB1; NFKB2; NFKB1A; NRAS; PAK1; PAK2; PAK3; PAK4; PIK3C2B; PIK3C3 (VPS34); PIK3CA; PIK3CB; PIK3CD; PIK3R1; PKCA; PKCB; PKCM; PKCQ; PLCY1; PRF1 (Perforin); PTEN; RAC1; RAF1; RELA; SDF1; SHP2; SLP76; SOS; SRC; TBK1; TCRA; TEC; TRAF6; VAV1; VAV2; or ZAP70.

### 4. Transmembrane Domain

In some embodiments, the CAR transmembrane domain comprises at least a transmembrane region of the alpha, beta or zeta chain of a T cell receptor, CD28, CD3 epsilon, CD45, CD4, CD5, CD8, CD9, CD16, CD22, CD33, CD37, CD64, CD80, CD86, CD134, CD137, CD154, or functional variant thereof. In some embodiments, the transmembrane domain comprises at least a transmembrane region(s) of CD8α, CD8β, 4-1BB/CD137, CD28, CD34, CD4, FcERIγ, CD16, OX40/CD134, CD3ζ, CD3ε, CD3γ, CD3δ, TCRα, TCRβ, TCRδ, CD32, CD64, CD64, CD45, CD5, CD9, CD22, CD37, CD80, CD86, CD40, CD40L/CD154, VEGFR2, FAS, and FGFR2B, or functional variant thereof. antigen binding domain binds

### 5. Signaling Domain or Plurality of Signaling Domains

In some embodiments, a CAR described herein comprises one or at least one signaling domain selected from one or more of B7-1/CD80; B7-2/CD86; B7-H1/PD-L1; B7-H2; B7-H3; B7-H4; B7-H6; B7-H7; BTLA/CD272; CD28; CTLA-4; Gi24/VISTA/B7-H5; ICOS/CD278; PD-1; PD-L2/B7-DC; PDCD6); 4-1BB/TNFSF9/CD137; 4-1BB Ligand/TNFSF9; BAFF/BLyS/TNFSF13B; BAFF R/TNFRSF13C; CD27/TNFRSF7; CD27 Ligand/TNFSF7; CD30/TNFRSF8; CD30 Ligand/TNFSF8; CD40/TNFRSF5; CD40/TNFSF5; CD40 Ligand/TNFSF5; DR3/TNFRSF25; GITR/TNFRSF18; GITR Ligand/TNFSF18; HVEM/TNFRSF14; LIGHT/TNFSF14; Lymphotoxin-alpha/TNF-beta; OX40/TNFRSF4; OX40 Ligand/TNFSF4; RELT/TNFRSF19L; TACI/TNFRSF13B; TL1A/TNFSF15; TNF-alpha; TNF RII/TNFRSF1B); 2B4/CD244/SLAMF4; BLAME/SLAMF8; CD2; CD2F-10/SLAMF9; CD48/SLAMF2; CD58/LFA-3; CD84/SLAMF5; CD229/SLAMF3; CRACC/SLAMF7; NTB-A/SLAMF6; SLAM/CD150); CD2; CD7; CD53; CD82/Kai-1; CD90/Thy1; CD96; CD160; CD200; CD300a/LMIR1; HLA Class I; HLA-DR; Ikaros; Integrin alpha 4/CD49d; Integrin alpha 4 beta 1; Integrin alpha 4 beta 7/LPAM-1; LAG-3; TCL1A; TCL1B; CRTAM; DAP12; Dectin-1/CLEC7A; DPPIV/CD26; EphB6; TIM-1/KIM-1/HAVCR; TIM-4; TSLP; TSLP R; lymphocyte function associated antigen-1 (LFA-1); NKG2C, a CD3 zeta domain, an immunoreceptor tyrosine-based activation motif (ITAM), CD27, CD28, 4-1BB, CD134/OX40, CD30, CD40, PD-1, ICOS, lymphocyte function-associated antigen-1 (LFA-1), CD2, CD7, LIGHT, NKG2C, B7-H3, a ligand that specifically binds with CD83, or functional fragment thereof.

In some embodiments, the at least one signaling domain comprises a CD3 zeta domain or an immunoreceptor tyrosine-based activation motif (ITAM), or functional variant thereof. In other embodiments, the at least one signaling domain comprises (i) a CD3 zeta domain, or an immunoreceptor tyrosine-based activation motif (ITAM), or functional variant thereof; and (ii) a CD28 domain, or a 4-1BB domain, or functional variant thereof. In yet other embodiments, the at least one signaling domain comprises a (i) a CD3 zeta domain, or an immunoreceptor tyrosine-based activation motif (ITAM), or functional variant thereof; (ii) a CD28 domain or functional variant thereof; and (iii) a 4-1BB domain, or a CD134 domain, or functional variant thereof. In some embodiments, the at least one signaling domain comprises a (i) a CD3 zeta domain, or an immunoreceptor tyrosine-based activation motif (ITAM), or functional variant thereof; (ii) a CD28 domain or functional variant thereof; (iii) a 4-1BB domain, or a CD134 domain, or functional variant thereof; and (iv) a cytokine or costimulatory ligand transgene.

In some embodiments, the at least two signaling domains comprise a CD3 zeta domain or an immunoreceptor tyrosine-based activation motif (ITAM), or functional variant thereof. In other embodiments, the at least two signaling domains comprise (i) a CD3 zeta domain, or an immunoreceptor tyrosine-based activation motif (ITAM), or functional variant thereof; and (ii) a CD28 domain, or a 4-1BB domain, or functional variant thereof. In yet other embodiments, the at least one signaling domain comprises a (i) a CD3 zeta domain, or an immunoreceptor tyrosine-based activation motif (ITAM), or functional variant thereof; (ii) a CD28 domain or functional variant thereof; and (iii) a 4-1BB domain, or a CD134 domain, or functional variant thereof. In some embodiments, the at least two signaling domains comprise a (i) a CD3 zeta domain, or an immunoreceptor tyrosine-based activation motif (ITAM), or functional variant thereof; (ii) a CD28 domain or functional variant thereof; (iii) a 4-1BB domain, or a CD134 domain, or functional variant thereof; and (iv) a cytokine or costimulatory ligand transgene.

In some embodiments, the at least three signaling domains comprise a CD3 zeta domain or an immunoreceptor tyrosine-based activation motif (ITAM), or functional variant thereof. In other embodiments, the at least three signaling domains comprise (i) a CD3 zeta domain, or an immunoreceptor tyrosine-based activation motif (ITAM), or functional variant thereof; and (ii) a CD28 domain, or a 4-1BB domain, or functional variant thereof. In yet other embodiments, the least three signaling domains comprises a (i) a CD3 zeta domain, or an immunoreceptor tyrosine-based activation motif (ITAM), or functional variant thereof; (ii) a CD28 domain or functional variant thereof; and (iii) a 4-1BB domain, or a CD134 domain, or functional variant thereof. In some embodiments, the at least three signaling domains comprise a (i) a CD3 zeta domain, or an immunoreceptor tyrosine-based activation motif (ITAM), or functional variant thereof; (ii) a CD28 domain or functional variant thereof; (iii) a 4-1BB domain, or a CD134 domain, or functional variant thereof; and (iv) a cytokine or costimulatory ligand transgene.

In some embodiments, the CAR comprises a CD3 zeta domain or an immunoreceptor tyrosine-based activation motif (ITAM), or functional variant thereof. In some embodiments, the CAR comprises (i) a CD3 zeta domain, or an immunoreceptor tyrosine-based activation motif (ITAM), or functional variant thereof; and (ii) a CD28 domain, or a 4-1BB domain, or functional variant thereof.

In some embodiments, the CAR comprises a (i) a CD3 zeta domain, or an immunoreceptor tyrosine-based activation motif (ITAM), or functional variant thereof; (ii) a CD28 domain or functional variant thereof; and (iii) a 4-1BB domain, or a CD134 domain, or functional variant thereof.

In some embodiments, the CAR comprises (i) a CD3 zeta domain, or an immunoreceptor tyrosine-based activation motif (ITAM), or functional variant thereof; (ii) a CD28 domain, or a 4-1BB domain, or functional variant thereof, and/or (iii) a 4-1BB domain, or a CD134 domain, or functional variant thereof.

In some embodiments, the CAR comprises a (i) a CD3 zeta domain, or an immunoreceptor tyrosine-based activation motif (ITAM), or functional variant thereof; (ii) a CD28 domain or functional variant thereof; (iii) a 4-1BB domain, or a CD134 domain, or functional variant thereof; and (iv) a cytokine or costimulatory ligand transgene. 6. Domain which upon successful signaling of the CAR induces expression of a cytokine gene

In some embodiments, a first, second, third, or fourth generation CAR further comprises a domain which upon successful signaling of the CAR induces expression of a cytokine gene. In some embodiments, a cytokine gene is endogenous or exogenous to a target cell comprising a CAR which comprises a domain which upon successful signaling of the CAR induces expression of a cytokine gene. In some embodiments, a cytokine gene encodes a pro-inflammatory cytokine. In some embodiments, a cytokine gene encodes IL-1, IL-2, IL-9, IL-12, IL-18, TNF, or IFN-gamma, or functional fragment thereof. In some embodiments, a domain which upon successful signaling of the CAR induces expression of a cytokine gene is or comprises a transcription factor or functional domain or fragment thereof. In some embodiments, a domain which upon successful signaling of the CAR induces expression of a cytokine gene is or comprises a transcription factor or functional domain or fragment thereof. In some embodiments, a transcription factor or functional domain or fragment thereof is or comprises a nuclear factor of activated T cells (NFAT), an NF-kB, or functional domain or fragment thereof. See, e.g., Zhang. C. et al., Engineering CAR-T cells. Biomarker Research. 5:22 (2017); WO 2016126608; Sha, H. et al. Chimaeric antigen receptor T-cell therapy for tumour immunotherapy. Bioscience Reports Jan. 27, 2017, 37 (1).

In some embodiments, the CAR further comprises one or more spacers, e.g., wherein the spacer is a first spacer between the antigen binding domain and the transmembrane domain. In some embodiments, the first spacer includes at least a portion of an immunoglobulin constant region or variant or modified version thereof. In some embodiments, the spacer is a second spacer between the transmembrane domain and a signaling domain. In some embodiments, the second spacer is an oligopeptide, e.g., wherein the oligopeptide comprises glycine and serine residues such as but not limited to glycine-serine doublets. In some embodiments, the CAR comprises two or more spacers, e.g., a spacer between the antigen binding domain and the transmembrane domain and a spacer between the transmembrane domain and a signaling domain.

In some embodiments, any one of the cells described herein comprises a nucleic acid encoding a CAR or a first generation CAR. In some embodiments, a first generation CAR comprises an antigen binding domain, a transmembrane domain, and signaling domain. In some embodiments, a signaling domain mediates downstream signaling during T cell activation.

In some embodiments, any one of the cells described herein comprises a nucleic acid encoding a CAR or a second generation CAR. In some embodiments, a second generation CAR comprises an antigen binding domain, a transmembrane domain, and two signaling domains. In some embodiments, a signaling domain mediates downstream signaling during T cell activation. In some embodiments, a signaling domain is a costimulatory domain. In some embodiments, a costimulatory domain enhances cytokine production, CAR-T cell proliferation, and/or CAR-T cell persistence during T cell activation.

In some embodiments, any one of the cells described herein comprises a nucleic acid encoding a CAR or a third generation CAR. In some embodiments, a third generation CAR comprises an antigen binding domain, a transmembrane domain, and at least three signaling domains. In some embodiments, a signaling domain mediates downstream signaling during T cell activation. In some embodiments, a signaling domain is a costimulatory domain. In some embodiments, a costimulatory domain enhances cytokine production, CAR-T cell proliferation, and or CAR-T cell persistence during T cell activation. In some embodiments, a third generation CAR comprises at least two costimulatory domains. In some embodiments, the at least two costimulatory domains are not the same.

In some embodiments, any one of the cells described herein comprises a nucleic acid encoding a CAR or a fourth generation CAR. In some embodiments, a fourth generation CAR comprises an antigen binding domain, a transmembrane domain, and at least two, three, or four signaling domains. In some embodiments, a signaling domain mediates downstream signaling during T cell activation. In some embodiments, a signaling domain is a costimulatory domain. In some embodiments, a costimulatory domain enhances cytokine production, CAR-T cell proliferation, and or CAR-T cell persistence during T cell activation.

### 7. ABD Comprising an Antibody or Antigen-Binding Portion Thereof

In some embodiments, a CAR antigen binding domain is or comprises an antibody or antigen-binding portion thereof. In some embodiments, a CAR antigen binding domain is or comprises an scFv or Fab. In some embodiments, a CAR antigen binding domain comprises an scFv or Fab fragment of a CD19 antibody; CD22 antibody; T-cell alpha chain antibody; T-cell β chain antibody; T-cell γ chain antibody; T-cell δ chain antibody; CCR7 antibody; CD3 antibody; CD4 antibody; CD5 antibody; CD7 antibody; CD8 antibody; CD11b antibody; CD11c antibody; CD16 antibody; CD20 antibody; CD21 antibody; CD25 antibody; CD28 antibody; CD34 antibody; CD35 antibody; CD40 antibody; CD45RA antibody; CD45RO antibody; CD52 antibody; CD56 antibody; CD62L antibody; CD68 antibody; CD80 antibody; CD95 antibody; CD117 antibody; CD127 antibody; CD133 antibody; CD137 (4-1 BB) antibody; CD163 antibody; F4/80 antibody; IL-4Ra antibody; Sca-1 antibody; CTLA-4 antibody; GITR antibody GARP antibody; LAP antibody; granzyme B antibody; LFA-1 antibody; MR1 antibody; uPAR antibody; or transferrin receptor antibody.

In some embodiments, a CAR comprises a signaling domain which is a costimulatory domain. In some embodiments, a CAR comprises a second costimulatory domain. In some embodiments, a CAR comprises at least two costimulatory domains. In some embodiments, a CAR comprises at least three costimulatory domains. In some embodiments, a CAR comprises a costimulatory domain selected from one or more of CD27, CD28, 4-1BB, CD134/OX40, CD30, CD40, PD-1, ICOS, lymphocyte function-associated antigen-1 (LFA-1), CD2, CD7, LIGHT, NKG2C, B7-H3, a ligand that specifically binds with CD83. In some embodiments, if a CAR comprises two or more costimulatory domains, two costimulatory domains are different. In some embodiments, if a CAR comprises two or more costimulatory domains, two costimulatory domains are the same.

In addition to the CARs described herein, various chimeric antigen receptors and nucleotide sequences encoding the same are known in the art and would be suitable for fusosomal delivery and reprogramming of target cells in vivo and in vitro as described herein. See, e.g., WO2013040557; WO2012079000; WO2016030414; Smith T, et al., Nature Nanotechnology. 2017. DOI: 10.1038/NNANO.2017.57, the disclosures of which are herein incorporated by reference.

### 8. Additional Descriptions of CARs

In certain embodiments, the cell may comprise an exogenous polynucleotide encoding a CAR. CARs (also known as chimeric immunoreceptors, chimeric T cell receptors, or artificial T cell receptors) are receptor proteins that have been engineered to give host cells (e.g., T cells) the new ability to target a specific protein. The receptors are chimeric because they combine both antigen-binding and T cell activating functions into a single receptor. The polycistronic vector of the present disclosure may be used to express one or more CARs in a host cell (e.g., a T cell) for use in cell-based therapies against various target antigens. The CARs expressed by the one or more expression cassettes may be the same or different. In these embodiments, the CAR may comprise an extracellular binding domain (also referred to as a “binder”) that specifically binds a target antigen, a transmembrane domain, and an intracellular signaling domain. In certain embodiments, the CAR may further comprise one or more additional elements, including one or more signal peptides, one or more extracellular hinge domains, and/or one or more intracellular costimulatory domains. Domains may be directly adjacent to one another, or there may be one or more amino acids linking the domains. The nucleotide sequence encoding a CAR may be derived from a mammalian sequence, for example, a mouse sequence, a primate sequence, a human sequence, or combinations thereof. In the cases where the nucleotide sequence encoding a CAR is non-human, the sequence of the CAR may be humanized. The nucleotide sequence encoding a CAR may also be codon-optimized for expression in a mammalian cell, for example, a human cell. In any of these embodiments, the nucleotide sequence encoding a CAR may be at least 80% identical (e.g., at least 80%, at least 85%, at least 90%, at least 95%, at least 96%, at least 97%, at least 98%, at least 99%, or 100% identical) to any of the nucleotide sequences disclosed herein. The sequence variations may be due to codon-optimalization, humanization, restriction enzyme-based cloning scars, and/or additional amino acid residues linking the functional domains, etc.

In certain embodiments, the CAR may comprise a signal peptide at the N-terminus. Non-limiting examples of signal peptides include CD8a signal peptide, IgK signal peptide, and granulocyte-macrophage colony-stimulating factor receptor subunit alpha (GMCSFR-α, also known as colony stimulating factor 2 receptor subunit alpha (CSF2RA)) signal peptide, and variants thereof, the amino acid sequences of which are provided in Table 3 below.

TABLE 3
Exemplary sequences of signal peptides

| SEQ ID NO: | Sequence | Description |
|---|---|---|
| 6 | MALPVTALLLPLALLLHAARP | CD8α signal peptide |
| 7 | METDTLLLWVLLLWVPGSTG | IgK signal peptide |
| 8 | MLLLVTSLLLCELPHPAFLLIP | GMCSFR-α (CSF2RA) signal peptide |

In certain embodiments, the extracellular binding domain of the CAR may comprise one or more antibodies specific to one target antigen or multiple target antigens. The antibody may be an antibody fragment, for example, an scFv, or a single-domain antibody fragment, for example, a VHH. In certain embodiments, the scFv may comprise a heavy chain variable region (V_{H}) and a light chain variable region (V_{L}) of an antibody connected by a linker. The V_{H} and the V_{L} may be connected in either order, i.e., V_{H}-linker-V_{L} or V_{L}-linker-V_{H}. Non-limiting examples of linkers include Whitlow linker, (G₄S)ₙ (n can be a positive integer, e.g., 1, 2, 3, 4, 5, 6, etc.) linker, and variants thereof. In certain embodiments, the antigen may be an antigen that is exclusively or preferentially expressed on tumor cells, or an antigen that is characteristic of an autoimmune or inflammatory disease. Exemplary target antigens include, but are not limited to, CD5, CD19, CD20, CD22, CD23, CD30, CD70, Kappa, Lambda, and B cell maturation agent (BCMA), G-protein coupled receptor family C group 5 member D (GPRC5D) (associated with leukemias); CS1/SLAMF7, CD38, CD138, GPRC5D, TACI, and BCMA (associated with myelomas); GD2, HER2, EGFR, EGFRvIII, B7H3, PSMA, PSCA, CAIX, CD171, CEA, CSPG4, EPHA2, FAP, FRa, IL-13Ra, Mesothelin, MUC1, MUC16, and ROR1 (associated with solid tumors), and CD79b. In any of these embodiments, the extracellular binding domain of the CAR can be codon-optimized for expression in a host cell or have variant sequences to increase functions of the extracellular binding domain.

In certain embodiments, the CAR may comprise a hinge domain, also referred to as a spacer. The terms “hinge” and “spacer” may be used interchangeably in the present disclosure. Non-limiting examples of hinge domains include CD8a hinge domain, CD28 hinge domain, IgG4 hinge domain, IgG4 hinge-CH2-CH3 domain, and variants thereof, the amino acid sequences of which are provided in Table 4 below.

TABLE 4
Exemplary sequences of hinge domains

| SEQ ID NO: | Sequence | Description |
|---|---|---|
| 9 | TTTPAPRPPTPAPTIASQPLSLRPEACRPAAGGAVHTRGLDFA | CD8α hinge domain |
| | CD | |
| 10 | IEVMYPPPYLDNEKSNGTIIHVKGKHLCPSPLFPGPSKP | CD28 hinge domain |
| 113 | AAAIEVMYPPPYLDNEKSNGTIIHVKGKHLCPSPLFPGPSKP | CD28 hinge domain |
| 11 | ESKYGPPCPPCP | IgG4 hinge domain |
| 12 | ESKYGPPCPSCP | IgG4 hinge domain |
| 13 | ESKYGPPCPPCPAPEFLGGPSVFLFPPKPKDTLMISRTPEVTCV | IgG4 hinge-CH2-CH3 |
| | VVDVSQEDPEVQFNWYVDGVEVHNAKTKPREEQFNSTYRVV | domain |
| | SVLTVLHQDWLNGKEYKCKVSNKGLPSSIEKTISKAKGQPREP | |
| | QVYTLPPSQEEMTKNQVSLTCLVKGFYPSDIAVEWESNGQPE | |
| | NNYKTTPPVLDSDGSFFLYSRLTVDKSRWQEGNVFSCSVMHE | |
| | ALHNHYTQKSLSLSLGK | |

In certain embodiments, the transmembrane domain of the CAR may comprise a transmembrane region of the alpha, beta, or zeta chain of a T cell receptor, CD28, CD3ε, CD45, CD4, CD5, CD8, CD9, CD16, CD22, CD33, CD37, CD64, CD80, CD86, CD134, CD137, CD154, or a functional variant thereof, including the human versions of each of these sequences. In other embodiments, the transmembrane domain may comprise a transmembrane region of CD8α, CD8β, 4-1BB/CD137, CD28, CD34, CD4, FcERIγ, CD16, OX40/CD134, CD3ζ, CD3ε, CD3γ, CD3δ, TCRα, TCRβ, TCRζ, CD32, CD64, CD64, CD45, CD5, CD9, CD22, CD37, CD80, CD86, CD40, CD40L/CD154, VEGFR2, FAS, and FGFR2B, or a functional variant thereof, including the human versions of each of these sequences. Table 5 provides the amino acid sequences of a few exemplary transmembrane domains.

TABLE 5
Exemplary sequences of transmembrane domains

| SEQ ID NO: | Sequence | Description |
|---|---|---|
| 14 | IYIWAPLAGTCGVLLLSLVITLYC | CD8α transmembrane domain |
| 15 | FWVLVVVGGVLACYSLLVTVAFIIFWV | CD28 transmembrane domain |
| 114 | MFWVLVVVGGVLACYSLLVTVAFIIFWV | CD28 transmembrane domain |

In certain embodiments, the intracellular signaling domain and/or intracellular costimulatory domain of the CAR may comprise one or more signaling domains selected from B7-1/CD80, B7-2/CD86, B7-H1/PD-L1, B7-H2, B7-H3, B7-H4, B7-H6, B7-H7, BTLA/CD272, CD28, CTLA-4, Gi24/VISTA/B7-H5, ICOS/CD278, PD-1, PD-L2/B7-DC, PDCD6, 4-1BB/TNFSF9/CD137, 4-1BB Ligand/TNFSF9, BAFF/BLyS/TNFSF13B, BAFF R/TNFRSF13C, CD27/TNFRSF7, CD27 Ligand/TNFSF7, CD30/TNFRSF8, CD30 Ligand/TNFSF8, CD40/TNFRSF5, CD40/TNFSF5, CD40 Ligand/TNFSF5, DR3/TNFRSF25, GITR/TNFRSF18, GITR Ligand/TNFSF18, HVEM/TNFRSF14, LIGHT/TNFSF14, Lymphotoxin-alpha/TNFβ, OX40/TNFRSF4, OX40 Ligand/TNFSF4, RELT/TNFRSF19L, TACI/TNFRSF13B, TL1A/TNFSF15, TNFα, TNF RII/TNFRSF1B, 2B4/CD244/SLAMF4, BLAME/SLAMF8, CD2, CD2F-10/SLAMF9, CD48/SLAMF2, CD58/LFA-3, CD84/SLAMF5, CD229/SLAMF3, CRACC/SLAMF7, NTB-A/SLAMF6, SLAM/CD150, CD2, CD7, CD53, CD82/Kai-1, CD90/Thy1, CD96, CD160, CD200, CD300a/LMIR1, HLA Class I, HLA-DR, Ikaros, Integrin alpha 4/CD49d, Integrin alpha 4 beta 1, Integrin alpha 4 beta 7/LPAM-1, LAG-3, TCL1A, TCL1B, CRTAM, DAP12, Dectin-1/CLEC7A, DPPIV/CD26, EphB6, TIM-1/KIM-1/HAVCR, TIM-4, TSLP, TSLP R, lymphocyte function associated antigen-1 (LFA-1), NKG2C, CD3ζ, an immunoreceptor tyrosine-based activation motif (ITAM), CD27, CD28, 4-1BB, CD134/OX40, CD30, CD40, PD-1, ICOS, lymphocyte function-associated antigen-1 (LFA-1), CD2, CD7, LIGHT, NKG2C, B7-H3, a ligand that specifically binds with CD83, and a functional variant thereof including the human versions of each of these sequences. In some embodiments, the intracellular signaling domain and/or intracellular costimulatory domain comprises one or more signaling domains selected from a CD3ζ domain, an ITAM, a CD28 domain, 4-1BB domain, or a functional variant thereof. Table 6 provides the amino acid sequences of a few exemplary intracellular costimulatory and/or signaling domains. In certain embodiments, as in the case of tisagenlecleucel as described below, the CD3ζ signaling domain of SEQ ID NO:18 may have a mutation, e.g., a glutamine (Q) to lysine (K) mutation, at amino acid position 14 (see SEQ ID NO:115).

TABLE 6
Exemplary sequences of intracellular costimulatory and/or signaling domains

| SEQ ID NO: | Sequence | Description |
|---|---|---|
| 16 | KRGRKKLLYIFKQPFMRPVQTTQEEDGCSCRFPEEEEG | 4-1BB costimulatory domain |
| | GCEL | |
| 17 | RSKRSRLLHSDYMNMTPRRPGPTRKHYQPYAPPRDFA | CD28 costimulatory domain |
| | AYRS | |
| 18 | RVKFSRSADAPAYQQGQNQLYNELNLGRREEYDVLDK | CD3ζ signaling domain |
| | RRGRDPEMGGKPRRKNPQEGLYNELQKDKMAEAYSEI | |
| | GMKGERRRGKGHDGLYQGLSTATKDTYDALHMQALP | |
| | PR | |
| 115 | RVKFSRSADAPAYKQGQNQLYNELNLGRREEYDVLDK | CD3ζ signaling domain (with Q |
| | RRGRDPEMGGKPRRKNPQEGLYNELQKDKMAEAYSEI | to K mutation at position 14) |
| | GMKGERRRGKGHDGLYQGLSTATKDTYDALHMQALP | |
| | PR | |

In certain embodiments where the polycistronic vector encodes two or more CARs, the two or more CARs may comprise the same functional domains, or one or more different functional domains, as described. For example, the two or more CARs may comprise different signal peptides, extracellular binding domains, hinge domains, transmembrane domains, costimulatory domains, and/or intracellular signaling domains, in order to minimize the risk of recombination due to sequence similarities. Or, alternatively, the two or more CARs may comprise the same domains. In the cases where the same domain(s) and/or backbone are used, it is optional to introduce codon divergence at the nucleotide sequence level to minimize the risk of recombination.

a. CD19 CAR

In some embodiments, the additional CAR is a CD19 CAR (“CD19-CAR”), and in these embodiments, the polycistronic vector comprises an expression cassette that contains a nucleotide sequence encoding a CD19 CAR. In some embodiments, the CD19 CAR may comprise a signal peptide, an extracellular binding domain that specifically binds CD19, a hinge domain, a transmembrane domain, an intracellular costimulatory domain, and/or an intracellular signaling domain in tandem.

In some embodiments, the signal peptide of the CD19 CAR comprises a CD8a signal peptide. In some embodiments, the CD8a signal peptide comprises or consists of an amino acid sequence set forth in SEQ ID NO:6 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:6. In some embodiments, the signal peptide comprises an IgK signal peptide. In some embodiments, the IgK signal peptide comprises or consists of an amino acid sequence set forth in SEQ ID NO:7 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:7. In some embodiments, the signal peptide comprises a GMCSFR-α or CSF2RA signal peptide. In some embodiments, the GMCSFR-α or CSF2RA signal peptide comprises or consists of an amino acid sequence set forth in SEQ ID NO:8 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:8.

In some embodiments, the extracellular binding domain of the CD19 CAR is specific to CD19, for example, human CD19. The extracellular binding domain of the CD19 CAR can be codon-optimized for expression in a host cell or to have variant sequences to increase functions of the extracellular binding domain. In some embodiments, the extracellular binding domain comprises an immunogenically active portion of an immunoglobulin molecule, for example, an scFv.

In some embodiments, the extracellular binding domain of the CD19 CAR comprises an scFv derived from the FMC63 monoclonal antibody (FMC63), which comprises the heavy chain variable region (V_{H}) and the light chain variable region (V_{L}) of FMC63 connected by a linker. FMC63 and the derived scFv have been described in Nicholson et al., Mol. Immun. 34(16-17):1157-1165 (1997) and PCT Application Publication No. WO2018/213337, the entire contents of each of which are incorporated by reference herein. In some embodiments, the amino acid sequences of the entire FMC63-derived scFv (also referred to as FMC63 scFv) and its different portions are provided in Table 7 below. In some embodiments, the CD19-specific scFv comprises or consists of an amino acid sequence set forth in SEQ ID NO:19, 20, or 25, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:19, 20, or 25. In some embodiments, the CD19-specific scFv may comprise one or more CDRs having amino acid sequences set forth in SEQ ID NOs: 21-23 and 26-28. In some embodiments, the CD19-specific scFv may comprise a light chain with one or more CDRs having amino acid sequences set forth in SEQ ID NOs: 21-23. In some embodiments, the CD19-specific scFv may comprise a heavy chain with one or more CDRs having amino acid sequences set forth in SEQ ID NOs: 26-28. In any of these embodiments, the CD19-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the CD19 CAR comprises or consists of the one or more CDRs as described herein.

In some embodiments, the linker linking the V_{H} and the V_{L} portions of the scFv is a Whitlow linker having an amino acid sequence set forth in SEQ ID NO:24. In some embodiments, the Whitlow linker may be replaced by a different linker, for example, a 3×G₄S linker having an amino acid sequence set forth in SEQ ID NO:30, which gives rise to a different FMC63-derived scFv having an amino acid sequence set forth in SEQ ID NO:29. In certain of these embodiments, the CD19-specific scFv comprises or consists of an amino acid sequence set forth in SEQ ID NO:29 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:29.

TABLE 7
Exemplary sequences of anti-CD19 scFv and components

| SEQ ID NO: | Amino Acid Sequence | Description |
|---|---|---|
| 19 | DIQMTQTTSSLSASLGDRVTISCRASQDISKYLN | Anti-CD19 FMC63 scFv |
| | WYQQKPDGTVKLLIYHTSRLHSGVPSRFSGSGSG | entire sequence, with |
| | TDYSLTISNLEQEDIATYFCQQGNTLPYTFGGGTK | Whitlow linker |
| | LEITGSTSGSGKPGSGEGSTKGEVKLQESGPGLV | |
| | APSQSLSVTCTVSGVSLPDYGVSWIRQPPRKGLE | |
| | WLGVIWGSETTYYNSALKSRLTIIKDNSKSQVFLK | |
| | MNSLQTDDTAIYYCAKHYYYGGSYAMDYWGQ | |
| | GTSVTVSS | |
| 20 | DIQMTQTTSSLSASLGDRVTISCRASQDISKYLN | Anti-CD19 FMC63 scFv light |
| | WYQQKPDGTVKLLIYHTSRLHSGVPSRFSGSGSG | chain variable region |
| | TDYSLTISNLEQEDIATYFCQQGNTLPYTFGGGTK | |
| | LEIT | |
| 21 | QDISKY | Anti-CD19 FMC63 scFv light |
| | | chain CDR1 |
| 22 | HTS | Anti-CD19 FMC63 scFv light |
| | | chain CDR2 |
| 23 | QQGNTLPYT | Anti-CD19 FMC63 scFv light |
| | | chain CDR3 |
| 24 | GSTSGSGKPGSGEGSTKG | Whitlow linker |
| 25 | EVKLQESGPGLVAPSQSLSVTCTVSGVSLPDYGV | Anti-CD19 FMC63 scFv |
| | SWIRQPPRKGLEWLGVIWGSETTYYNSALKSRLT | heavy chain variable |
| | IIKDNSKSQVFLKMNSLQTDDTAIYYCAKHYYYG | region |
| | GSYAMDYWGQGTSVTVSS | |
| 26 | GVSLPDYG | Anti-CD19 FMC63 scFv |
| | | heavy chain CDR1 |
| 27 | IWGSETT | Anti-CD19 FMC63 scFv |
| | | heavy chain CDR2 |
| 28 | AKHYYYGGSYAMDY | Anti-CD19 FMC63 scFv |
| | | heavy chain CDR3 |
| 29 | DIQMTQTTSSLSASLGDRVTISCRASQDISKYLN | Anti-CD19 FMC63 scFv |
| | WYQQKPDGTVKLLIYHTSRLHSGVPSRFSGSGSG | entire sequence, with 3xG₄S |
| | TDYSLTISNLEQEDIATYFCQQGNTLPYTFGGGTK | linker |
| | LEITGGGGSGGGGSGGGGSEVKLQESGPGLVAP | |
| | SQSLSVTCTVSGVSLPDYGVSWIRQPPRKGLEW | |
| | LGVIWGSETTYYNSALKSRLTIIKDNSKSQVFLKM | |
| | NSLQTDDTAIYYCAKHYYYGGSYAMDYWGQGT | |
| | SVTVSS | |
| 30 | GGGGSGGGGSGGGGS | 3xG₄S linker |

In some embodiments, the extracellular binding domain of the CD19 CAR is derived from an antibody specific to CD19, including, for example, SJ25C1 (Bejcek et al., Cancer Res. 55:2346-2351 (1995)), HD37 (Pezutto et al., J. Immunol. 138(9):2793-2799 (1987)), 4G7 (Meeker et al., Hybridoma 3:305-320 (1984)), B43 (Bejcek (1995)), BLY3 (Bejcek (1995)), B4 (Freedman et al., 70:418-427 (1987)), B4 HB12b (Kansas & Tedder, J. Immunol. 147:4094-4102 (1991); Yazawa et al., Proc. Natl. Acad. Sci. USA 102:15178-15183 (2005); Herbst et al., J. Pharmacol. Exp. Ther. 335:213-222 (2010)), BU12 (Callard et al., J. Immunology, 148(10): 2983-2987 (1992)), and CLB-CD19 (De Rie Cell. Immunol. 118:368-381(1989)). In any of these embodiments, the extracellular binding domain of the CD19 CAR can comprise or consist of the V_{H}, the V_{L}, and/or one or more CDRs of any of the antibodies.

In some embodiments, the hinge domain of the CD19 CAR comprises a CD8a hinge domain, for example, a human CD8a hinge domain. In some embodiments, the CD8a hinge domain comprises or consists of an amino acid sequence set forth in SEQ ID NO:9 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:9. In some embodiments, the hinge domain comprises a CD28 hinge domain, for example, a human CD28 hinge domain. In some embodiments, the CD28 hinge domain comprises or consists of an amino acid sequence set forth in SEQ ID NO:10 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:10. In some embodiments, the hinge domain comprises an IgG4 hinge domain, for example, a human IgG4 hinge domain. In some embodiments, the IgG4 hinge domain comprises or consists of an amino acid sequence set forth in SEQ ID NO:11 or SEQ ID NO:12, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:11 or SEQ ID NO:12. In some embodiments, the hinge domain comprises a IgG4 hinge-Ch2-Ch3 domain, for example, a human IgG4 hinge-Ch2-Ch3 domain. In some embodiments, the IgG4 hinge-Ch2-Ch3 domain comprises or consists of an amino acid sequence set forth in SEQ ID NO:13 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:13.

In some embodiments, the transmembrane domain of the CD19 CAR comprises a CD8a transmembrane domain, for example, a human CD8a transmembrane domain. In some embodiments, the CD8a transmembrane domain comprises or consists of an amino acid sequence set forth in SEQ ID NO:14 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:14. In some embodiments, the transmembrane domain comprises a CD28 transmembrane domain, for example, a human CD28 transmembrane domain. In some embodiments, the CD28 transmembrane domain comprises or consists of an amino acid sequence set forth in SEQ ID NO:15 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:15.

In some embodiments, the intracellular costimulatory domain of the CD19 CAR comprises a 4-1BB costimulatory domain. 4-1BB, also known as CD137, transmits a potent costimulatory signal to T cells, promoting differentiation and enhancing long-term survival of T lymphocytes. In some embodiments, the 4-1BB costimulatory domain is human. In some embodiments, the 4-1BB costimulatory domain comprises or consists of an amino acid sequence set forth in SEQ ID NO:16 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:16. In some embodiments, the intracellular costimulatory domain comprises a CD28 costimulatory domain. CD28 is another co-stimulatory molecule on T cells. In some embodiments, the CD28 costimulatory domain is human. In some embodiments, the CD28 costimulatory domain comprises or consists of an amino acid sequence set forth in SEQ ID NO:17 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:17. In some embodiments, the intracellular costimulatory domain of the CD19 CAR comprises a 4-1BB costimulatory domain and a CD28 costimulatory domain as described.

In some embodiments, the intracellular signaling domain of the CD19 CAR comprises a CD3 zeta (ζ) signaling domain. CD3ζ associates with TCRs to produce a signal and contains immunoreceptor tyrosine-based activation motifs (ITAMs). The CD3ζ signaling domain refers to amino acid residues from the cytoplasmic domain of the zeta chain that are sufficient to functionally transmit an initial signal necessary for T cell activation. In some embodiments, the CD3ζ signaling domain is human. In some embodiments, the CD3ζ signaling domain comprises or consists of an amino acid sequence set forth in SEQ ID NO:18 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:18.

In some embodiments, the polycistronic vector comprises an expression cassette that contains a nucleotide sequence encoding a CD19 CAR, including, for example, a CD19 CAR comprising the CD19-specific scFv having sequences set forth in SEQ ID NO:19 or SEQ ID NO:29, the CD8a hinge domain of SEQ ID NO:9, the CD8a transmembrane domain of SEQ ID NO:14, the 4-1BB costimulatory domain of SEQ ID NO:16, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof. In any of these embodiments, the CD19 CAR may additionally comprise a signal peptide (e.g., a CD8a signal peptide) as described.

In some embodiments, the polycistronic vector comprises an expression cassette that contains a nucleotide sequence encoding a CD19 CAR, including, for example, a CD19 CAR comprising the CD19-specific scFv having sequences set forth in SEQ ID NO:19 or SEQ ID NO:29, the IgG4 hinge domain of SEQ ID NO:11 or SEQ ID NO:12, the CD28 transmembrane domain of SEQ ID NO:15, the 4-1BB costimulatory domain of SEQ ID NO:16, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof. In any of these embodiments, the CD19 CAR may additionally comprise a signal peptide (e.g., a CD8a signal peptide) as described.

In some embodiments, the polycistronic vector comprises an expression cassette that contains a nucleotide sequence encoding a CD19 CAR, including, for example, a CD19 CAR comprising the CD19-specific scFv having sequences set forth in SEQ ID NO:19 or SEQ ID NO:29, the CD28 hinge domain of SEQ ID NO:10, the CD28 transmembrane domain of SEQ ID NO:15, the CD28 costimulatory domain of SEQ ID NO:17, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof. In any of these embodiments, the CD19 CAR may additionally comprise a signal peptide (e.g., a CD8a signal peptide) as described.

In some embodiments, the polycistronic vector comprises an expression cassette that contains a nucleotide sequence encoding a CD19 CAR as set forth in SEQ ID NO:116 or is at least 80% identical (e.g., at least 80%, at least 85%, at least 90%, at least 95%, at least 96%, at least 97%, at least 98%, at least 99%, or 100% identical) to the nucleotide sequence set forth in SEQ ID NO:116 (see Table 8). The encoded CD19 CAR has a corresponding amino acid sequence set forth in SEQ ID NO:117 or is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:117, with the following components: CD8a signal peptide, FMC63 scFv (V_{L}—Whitlow linker-V_{H}), CD8a hinge domain, CD8a transmembrane domain, 4-1BB costimulatory domain, and CD3ζ signaling domain.

In some embodiments, the polycistronic vector comprises an expression cassette that contains a nucleotide sequence encoding a commercially available embodiment of CD19 CAR. Non-limiting examples of commercially available embodiments of CD19 CARs expressed and/or encoded by T cells include tisagenlecleucel, lisocabtagene maraleucel, axicabtagene ciloleucel, and brexucabtagene autoleucel.

In some embodiments, the polycistronic vector comprises an expression cassette that contains a nucleotide sequence encoding tisagenlecleucel or portions thereof. Tisagenlecleucel comprises a CD19 CAR with the following components: CD8a signal peptide, FMC63 scFv (V_{L}-3×G₄S linker-V_{H}), CD8a hinge domain, CD8a transmembrane domain, 4-1BB costimulatory domain, and CD3ζ signaling domain. The nucleotide and amino acid sequence of the CD19 CAR in tisagenlecleucel are provided in Table 8, with annotations of the sequences provided in Table 9.

In some embodiments, the polycistronic vector comprises an expression cassette that contains a nucleotide sequence encoding lisocabtagene maraleucel or portions thereof. Lisocabtagene maraleucel comprises a CD19 CAR with the following components: GMCSFR-α or CSF2RA signal peptide, FMC63 scFv (V_{L}—Whitlow linker-V_{H}), IgG4 hinge domain, CD28 transmembrane domain, 4-1BB costimulatory domain, and CD3ζ signaling domain. The nucleotide and amino acid sequence of the CD19 CAR in lisocabtagene maraleucel are provided in Table 8, with annotations of the sequences provided in Table 10.

In some embodiments, the polycistronic vector comprises an expression cassette that contains a nucleotide sequence encoding axicabtagene ciloleucel or portions thereof. Axicabtagene ciloleucel comprises a CD19 CAR with the following components: GMCSFR-α or CSF2RA signal peptide, FMC63 scFv (V_{L}—Whitlow linker-V_{H}), CD28 hinge domain, CD28 transmembrane domain, CD28 costimulatory domain, and CD3ζ signaling domain. The nucleotide and amino acid sequence of the CD19 CAR in axicabtagene ciloleucel are provided in Table 8, with annotations of the sequences provided in Table 11.

In some embodiments, the polycistronic vector comprises an expression cassette that contains a nucleotide sequence encoding brexucabtagene autoleucel or portions thereof. Brexucabtagene autoleucel comprises a CD19 CAR with the following components: GMCSFR-α signal peptide, FMC63 scFv, CD28 hinge domain, CD28 transmembrane domain, CD28 costimulatory domain, and CD3 signaling domain.

In some embodiments, the polycistronic vector comprises an expression cassette that contains a nucleotide sequence encoding a CD19 CAR as set forth in SEQ ID NO: 31, 33, or 35, or is at least 80% identical (e.g., at least 80%, at least 85%, at least 90%, at least 95%, at least 96%, at least 97%, at least 98%, at least 99%, or 100% identical) to the nucleotide sequence set forth in SEQ ID NO: 31, 33, or 35. The encoded CD19 CAR has a corresponding amino acid sequence set forth in SEQ ID NO: 32, 34, or 36, respectively, or is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO: 32, 34, or 36, respectively.

TABLE 8
Exemplary sequences of CD19 CARs

| SEQ ID NO: | Sequence | Description |
|---|---|---|
| 116 | atggccttaccagtgaccgccttgctcctgccgctggccttgctgctccac | Exemplary CD19 |
| | gccgccaggccggacatccagatgacacagactacatcctccctgtctg | CAR nucleotide |
| | cctctctgggagacagagtcaccatcagttgcagggcaagtcaggacat | sequence |
| | tagtaaatatttaaattggtatcagcagaaaccagatggaactgttaaa | |
| | ctcctgatctaccatacatcaagattacactcaggagtcccatcaaggtt | |
| | cagtggcagtgggtctggaacagattattctctcaccattagcaacctgg | |
| | agcaagaagatattgccacttacttttgccaacagggtaatacgcttccg | |
| | tacacgttcggaggggggaccaagctggagatcacaggctccacctctg | |
| | gatccggcaagcccggatctggcgagggatccaccaagggcgaggtga | |
| | aactgcaggagtcaggacctggcctggtggcgccctcacagagcctgtc | |
| | cgtcacatgcactgtctcaggggtctcattacccgactatggtgtaagct | |
| | ggattcgccagcctccacgaaagggtctggagtggctgggagtaatatg | |
| | gggtagtgaaaccacatactataattcagctctcaaatccagactgacc | |
| | atcatcaaggacaactccaagagccaagttttcttaaaaatgaacagtc | |
| | tgcaaactgatgacacagccatttactactgtgccaaacattattactac | |
| | ggtggtagctatgctatggactactggggccaaggaacctcagtcaccg | |
| | tctcctcaaccacgacgccagcgccgcgaccaccaacaccggcgccca | |
| | ccatcgcgtcgcagcccctgtccctgcgcccagaggcgtgccggccagc | |
| | ggcggggggcgcagtgcacacgagggggctggacttcgcctgtgatatc | |
| | tacatctgggcgcccttggccgggacttgtggggtccttctcctgtcactg | |
| | gttatcaccctttactgcaaacggggcagaaagaaactcctgtatatatt | |
| | caaacaaccatttatgagaccagtacaaactactcaagaggaagatgg | |
| | ctgtagctgccgatttccagaagaagaagaaggaggatgtgaactgag | |
| | agtgaagttcagcaggagcgcagacgcccccgcgtaccagcagggcca | |
| | gaaccagctctataacgagctcaatctaggacgaagagaggagtacga | |
| | tgttttggacaagagacgtggccgggaccctgagatggggggaaagcc | |
| | gagaaggaagaaccctcaggaaggcctgtacaatgaactgcagaaag | |
| | ataagatggcggaggcctacagtgagattgggatgaaaggcgagcgcc | |
| | ggaggggcaaggggcacgatggcctttaccagggtctcagtacagcca | |
| | ccaaggacacctacgacgcccttcacatgcaggccctgccccctcgc | |
| 117 | MALPVTALLLPLALLLHAARPDIQMTQTTSSLSASLGDRV | Exemplary CD19 |
| | TISCRASQDISKYLNWYQQKPDGTVKLLIYHTSRLHSGVPS | CAR amino acid |
| | RFSGSGSGTDYSLTISNLEQEDIATYFCQQGNTLPYTFGGG | sequence |
| | TKLEITGSTSGSGKPGSGEGSTKGEVKLQESGPGLVAPSQS | |
| | LSVTCTVSGVSLPDYGVSWIRQPPRKGLEWLGVIWGSET | |
| | TYYNSALKSRLTIIKDNSKSQVFLKMNSLQTDDTAIYYCAK | |
| | HYYYGGSYAMDYWGQGTSVTVSSTTTPAPRPPTPAPTIA | |
| | SQPLSLRPEACRPAAGGAVHTRGLDFACDIYIWAPLAGTC | |
| | GVLLLSLVITLYCKRGRKKLLYIFKQPFMRPVQTTQEEDGC | |
| | SCRFPEEEEGGCELRVKFSRSADAPAYQQGQNQLYNELN | |
| | LGRREEYDVLDKRRGRDPEMGGKPRRKNPQEGLYNELQ | |
| | KDKMAEAYSEIGMKGERRRGKGHDGLYQGLSTATKDTY | |
| | DALHMQALPPR | |
| 19 | DIQMTQTTSSLSASLGDRVTISCRASQDISKYLNWYQQKP | Exemplary CD19 |
| | DGTVKLLIYHTSRLHSGVPSRFSGSGSGTDYSLTISNLEQED | CAR scFv amino acid |
| | IATYFCQQGNTLPYTFGGGTKLEITGSTSGSGKPGSGEGST | sequence |
| | KGEVKLQESGPGLVAPSQSLSVTCTVSGVSLPDYGVSWIR | |
| | QPPRKGLEWLGVIWGSETTYYNSALKSRLTIIKDNSKSQVF | |
| | LKMNSLQTDDTAIYYCAKHYYYGGSYAMDYWGQGTSVT | |
| | VSS | |
| 25 | EVKLQESGPGLVAPSQSLSVTCTVSGVSLPDYGVSWIRQP | Exemplary CD19 |
| | PRKGLEWLGVIWGSETTYYNSALKSRLTIIKDNSKSQVFLK | CAR HC Variable |
| | MNSLQTDDTAIYYCAKHYYYGGSYAMDYWGQGTSVTVS | Region amino acid |
| | S | sequence |
| 26 | GVSLPDYG | Exemplary CD19 |
| | | CAR HC CDR1 amino |
| | | acid sequence |
| 27 | IWGSETT | Exemplary CD19 |
| | | CAR HC CDR2 amino |
| | | acid sequence |
| 28 | AKHYYYGGSYAMDY | Exemplary CD19 |
| | | CAR HC CDR3 amino |
| | | acid sequence |
| 20 | DIQMTQTTSSLSASLGDRVTISCRASQDISKYLNWYQQKP | Exemplary CD19 |
| | DGTVKLLIYHTSRLHSGVPSRFSGSGSGTDYSLTISNLEQED | CAR LC Variable |
| | IATYFCQQGNTLPYTFGGGTKLEIT | Region amino acid |
| | | sequence |
| 21 | QDISKY | Exemplary CD19 |
| | | CAR LC CDR1 amino |
| | | acid sequence |
| 22 | HTS | Exemplary CD19 |
| | | CAR LC CDR2 amino |
| | | acid sequence |
| 23 | QQGNTLPYT | Exemplary CD19 |
| | | CAR LC CDR3 amino |
| | | acid sequence |
| 31 | atggccttaccagtgaccgccttgctcctgccgctggccttgctgctccac | Tisagenlecleucel |
| | gccgccaggccggacatccagatgacacagactacatcctccctgtctg | CD19 CAR |
| | cctctctgggagacagagtcaccatcagttgcagggcaagtcaggacat | nucleotide sequence |
| | tagtaaatatttaaattggtatcagcagaaaccagatggaactgttaaa | |
| | ctcctgatctaccatacatcaagattacactcaggagtcccatcaaggtt | |
| | cagtggcagtgggtctggaacagattattctctcaccattagcaacctgg | |
| | agcaagaagatattgccacttacttttgccaacagggtaatacgcttccg | |
| | tacacgttcggaggggggaccaagctggagatcacaggtggcggtggc | |
| | tcgggcggtggtgggtcgggtggcggcggatctgaggtgaaactgcag | |
| | gagtcaggacctggcctggtggcgccctcacagagcctgtccgtcacat | |
| | gcactgtctcaggggtctcattacccgactatggtgtaagctggattcgc | |
| | cagcctccacgaaagggtctggagtggctgggagtaatatggggtagtg | |
| | aaaccacatactataattcagctctcaaatccagactgaccatcatcaag | |
| | gacaactccaagagccaagttttcttaaaaatgaacagtctgcaaactg | |
| | atgacacagccatttactactgtgccaaacattattactacggtggtagc | |
| | tatgctatggactactggggccaaggaacctcagtcaccgtctcctcaac | |
| | cacgacgccagcgccgcgaccaccaacaccggcgcccaccatcgcgtc | |
| | gcagcccctgtccctgcgcccagaggcgtgccggccagcggcgggggg | |
| | cgcagtgcacacgagggggctggacttcgcctgtgatatctacatctggg | |
| | cgcccttggccgggacttgtggggtccttctcctgtcactggttatcaccct | |
| | ttactgcaaacggggcagaaagaaactcctgtatatattcaaacaacca | |
| | tttatgagaccagtacaaactactcaagaggaagatggctgtagctgcc | |
| | gatttccagaagaagaagaaggaggatgtgaactgagagtgaagttca | |
| | gcaggagcgcagacgcccccgcgtacaagcagggccagaaccagctct | |
| | ataacgagctcaatctaggacgaagagaggagtacgatgttttggaca | |
| | agagacgtggccgggaccctgagatggggggaaagccgagaaggaag | |
| | aaccctcaggaaggcctgtacaatgaactgcagaaagataagatggcg | |
| | gaggcctacagtgagattgggatgaaaggcgagcgccggaggggcaa | |
| | ggggcacgatggcctttaccagggtctcagtacagccaccaaggacacc | |
| | tacgacgcccttcacatgcaggccctgccccctcgc | |
| 32 | MALPVTALLLPLALLLHAARPDIQMTQTTSSLSASLGDRV | Tisagenlecleucel |
| | TISCRASQDISKYLNWYQQKPDGTVKLLIYHTSRLHSGVPS | CD19 CAR amino |
| | RFSGSGSGTDYSLTISNLEQEDIATYFCQQGNTLPYTFGGG | acid sequence |
| | TKLEITGGGGSGGGGSGGGGSEVKLQESGPGLVAPSQSL | |
| | SVTCTVSGVSLPDYGVSWIRQPPRKGLEWLGVIWGSETT | |
| | YYNSALKSRLTIIKDNSKSQVFLKMNSLQTDDTAIYYCAKH | |
| | YYYGGSYAMDYWGQGTSVTVSSTTTPAPRPPTPAPTIAS | |
| | QPLSLRPEACRPAAGGAVHTRGLDFACDIYIWAPLAGTC | |
| | GVLLLSLVITLYCKRGRKKLLYIFKQPFMRPVQTTQEEDGC | |
| | SCRFPEEEEGGCELRVKFSRSADAPAYKQGQNQLYNELNL | |
| | GRREEYDVLDKRRGRDPEMGGKPRRKNPQEGLYNELQK | |
| | DKMAEAYSEIGMKGERRRGKGHDGLYQGLSTATKDTYD | |
| | ALHMQALPPR | |
| 29 | DIQMTQTTSSLSASLGDRVTISCRASQDISKYLNWYQQKP | Tisagenlecleucel |
| | DGTVKLLIYHTSRLHSGVPSRFSGSGSGTDYSLTISNLEQED | CD19 CAR scFv |
| | IATYFCQQGNTLPYTFGGGTKLEITGGGGSGGGGGGGG | amino acid |
| | SEVKLQESGPGLVAPSQSLSVTCTVSGVSLPDYGVSWIRQ | sequence |
| | PPRKGLEWLGVIWGSETTYYNSALKSRLTIIKDNSKSQVFL | |
| | KMNSLQTDDTAIYYCAKHYYYGGSYAMDYWGQGTSVTV | |
| | SS | |
| 25 | EVKLQESGPGLVAPSQSLSVTCTVSGVSLPDYGVSWIRQP | Tisagenlecleucel |
| | PRKGLEWLGVIWGSETTYYNSALKSRLTIIKDNSKSQVFLK | CD19 HC Variable |
| | MNSLQTDDTAIYYCAKHYYYGGSYAMDYWGQGTSVTVS | Region amino acid |
| | S | sequence |
| 26 | GVSLPDYG | Tisagenlecleucel |
| | | CD19 HC CDR1 |
| | | amino acid |
| | | sequence |
| 27 | IWGSETT | Tisagenlecleucel |
| | | CD19 HC CDR2 |
| | | amino acid |
| | | sequence |
| 28 | AKHYYYGGSYAMDY | Tisagenlecleucel |
| | | CD19 HC CDR3 |
| | | amino acid |
| | | sequence |
| 20 | DIQMTQTTSSLSASLGDRVTISCRASQDISKYLNWYQQKP | Tisagenlecleucel |
| | DGTVKLLIYHTSRLHSGVPSRFSGSGSGTDYSLTISNLEQED | CD19 LC Variable |
| | IATYFCQQGNTLPYTFGGGTKLEIT | Region amino acid |
| | | sequence |
| 21 | QDISKY | Tisagenlecleucel |
| | | CD19 LC CDR1 |
| | | amino acid |
| | | sequence |
| 22 | HTS | Tisagenlecleucel |
| | | CD19 LC CDR2 |
| | | amino acid |
| | | sequence |
| 23 | QQGNTLPYT | Tisagenlecleucel |
| | | CD19 LC CDR3 |
| | | amino acid |
| | | sequence |
| 33 | atgctgctgctggtgaccagcctgctgctgtgcgagctgccccaccccgc | Lisocabtagene |
| | ctttctgctgatccccgacatccagatgacccagaccacctccagcctga | maraleucel CD19 |
| | gcgccagcctgggcgaccgggtgaccatcagctgccgggccagccagg | CAR nucleotide |
| | acatcagcaagtacctgaactggtatcagcagaagcccgacggcaccg | sequence |
| | tcaagctgctgatctaccacaccagccggctgcacagcggcgtgcccag | |
| | ccggtttagcggcagcggctccggcaccgactacagcctgaccatctcc | |
| | aacctggaacaggaagatatcgccacctacttttgccagcagggcaaca | |
| | cactgccctacacctttggcggcggaacaaagctggaaatcaccggcag | |
| | cacctccggcagcggcaagcctggcagcggcgagggcagcaccaagg | |
| | gcgaggtgaagctgcaggaaagcggccctggcctggtggcccccagcc | |
| | agagcctgagcgtgacctgcaccgtgagcggcgtgagcctgcccgacta | |
| | cggcgtgagctggatccggcagccccccaggaagggcctggaatggct | |
| | gggcgtgatctggggcagcgagaccacctactacaacagcgccctgaa | |
| | gagccggctgaccatcatcaaggacaacagcaagagccaggtgttcct | |
| | gaagatgaacagcctgcagaccgacgacaccgccatctactactgcgc | |
| | caagcactactactacggcggcagctacgccatggactactggggccag | |
| | ggcaccagcgtgaccgtgagcagcgaatctaagtacggaccgccctgc | |
| | cccccttgccctatgttctgggtgctggtggtggtcggaggcgtgctggcc | |
| | tgctacagcctgctggtcaccgtggccttcatcatcttttgggtgaaacgg | |
| | ggcagaaagaaactcctgtatatattcaaacaaccatttatgagaccag | |
| | tacaaactactcaagaggaagatggctgtagctgccgatttccagaaga | |
| | agaagaaggaggatgtgaactgcgggtgaagttcagcagaagcgccg | |
| | acgcccctgcctaccagcagggccagaatcagctgtacaacgagctga | |
| | acctgggcagaagggaagagtacgacgtcctggataagcggagaggc | |
| | cgggaccctgagatgggcggcaagcctcggcggaagaacccccagga | |
| | aggcctgtataacgaactgcagaaagacaagatggccgaggcctacag | |
| | cgagatcggcatgaagggcgagcggaggcggggcaagggccacgacg | |
| | gcctgtatcagggcctgtccaccgccaccaaggatacctacgacgccct | |
| | gcacatgcaggccctgcccccaagg | |
| 34 | MLLLVTSLLLCELPHPAFLLIPDIQMTQTTSSLSASLGDRVT | Lisocabtagene |
| | ISCRASQDISKYLNWYQQKPDGTVKLLIYHTSRLHSGVPSR | maraleucel CD19 |
| | FSGSGSGTDYSLTISNLEQEDIATYFCQQGNTLPYTFGGGT | CAR amino acid |
| | KLEITGSTSGSGKPGSGEGSTKGEVKLQESGPGLVAPSQSL | sequence |
| | SVTCTVSGVSLPDYGVSWIRQPPRKGLEWLGVIWGSETT | |
| | YYNSALKSRLTIIKDNSKSQVFLKMNSLQTDDTAIYYCAKH | |
| | YYYGGSYAMDYWGQGTSVTVSSESKYGPPCPPCPMFW | |
| | VLVVVGGVLACYSLLVTVAFIIFWVKRGRKKLLYIFKQPFM | |
| | RPVQTTQEEDGCSCRFPEEEEGGCELRVKFSRSADAPAYQ | |
| | QGQNQLYNELNLGRREEYDVLDKRRGRDPEMGGKPRRK | |
| | NPQEGLYNELQKDKMAEAYSEIGMKGERRRGKGHDGLY | |
| | QGLSTATKDTYDALHMQALPPR | |
| 19 | DIQMTQTTSSLSASLGDRVTISCRASQDISKYLNWYQQKP | Lisocabtagene |
| | DGTVKLLIYHTSRLHSGVPSRFSGSGSGTDYSLTISNLEQED | maraleucel CD19 |
| | IATYFCQQGNTLPYTFGGGTKLEITGSTSGSGKPGSGEGST | CAR scFv amino acid |
| | KGEVKLQESGPGLVAPSQSLSVTCTVSGVSLPDYGVSWIR | sequence |
| | QPPRKGLEWLGVIWGSETTYYNSALKSRLTIIKDNSKSQVF | |
| | LKMNSLQTDDTAIYYCAKHYYYGGSYAMDYWGQGTSVT | |
| | VSS | |
| 25 | EVKLQESGPGLVAPSQSLSVTCTVSGVSLPDYGVSWIRQP | Lisocabtagene |
| | PRKGLEWLGVIWGSETTYYNSALKSRLTIIKDNSKSQVFLK | maraleucel CD19 |
| | MNSLQTDDTAIYYCAKHYYYGGSYAMDYWGQGTSVTVS | CAR HC Variable |
| | S | Region amino acid |
| | | sequence |
| 26 | GVSLPDYG | Lisocabtagene |
| | | maraleucel CD19 |
| | | CAR HC CDR1 amino |
| | | acid sequence |
| 27 | IWGSETT | Lisocabtagene |
| | | maraleucel CD19 |
| | | CAR HC CDR2 amino |
| | | acid sequence |
| 28 | AKHYYYGGSYAMDY | Lisocabtagene |
| | | maraleucel CD19 |
| | | CAR HC CDR3 amino |
| | | acid sequence |
| 20 | DIQMTQTTSSLSASLGDRVTISCRASQDISKYLNWYQQKP | Lisocabtagene |
| | DGTVKLLIYHTSRLHSGVPSRFSGSGSGTDYSLTISNLEQED | maraleucel CD19 |
| | IATYFCQQGNTLPYTFGGGTKLEIT | CAR LC Variable |
| | | Region amino acid |
| | | sequence |
| 21 | QDISKY | Lisocabtagene |
| | | maraleucel CD19 |
| | | CAR LC CDR1 amino |
| | | acid sequence |
| 22 | HTS | Lisocabtagene |
| | | maraleucel CD19 |
| | | CAR LC CDR2 amino |
| | | acid sequence |
| 23 | QQGNTLPYT | Lisocabtagene |
| | | maraleucel CD19 |
| | | CAR LC CDR3 amino |
| | | acid sequence |
| 35 | atgcttctcctggtgacaagccttctgctctgtgagttaccacacccagca | Axicabtagene |
| | ttcctcctgatcccagacatccagatgacacagactacatcctccctgtct | ciloleucel CD19 CAR |
| | gcctctctgggagacagagtcaccatcagttgcagggcaagtcaggaca | nucleotide sequence |
| | ttagtaaatatttaaattggtatcagcagaaaccagatggaactgttaaa | |
| | ctcctgatctaccatacatcaagattacactcaggagtcccatcaaggtt | |
| | cagtggcagtgggtctggaacagattattctctcaccattagcaacctgg | |
| | agcaagaagatattgccacttacttttgccaacagggtaatacgcttccg | |
| | tacacgttcggaggggggactaagttggaaataacaggctccacctctg | |
| | gatccggcaagcccggatctggcgagggatccaccaagggcgaggtga | |
| | aactgcaggagtcaggacctggcctggtggcgccctcacagagcctgtc | |
| | cgtcacatgcactgtctcaggggtctcattacccgactatggtgtaagct | |
| | ggattcgccagcctccacgaaagggtctggagtggctgggagtaatatg | |
| | gggtagtgaaaccacatactataattcagctctcaaatccagactgacc | |
| | atcatcaaggacaactccaagagccaagttttcttaaaaatgaacagtc | |
| | tgcaaactgatgacacagccatttactactgtgccaaacattattactac | |
| | ggtggtagctatgctatggactactggggtcaaggaacctcagtcaccgt | |
| | ctcctcagcggccgcaattgaagttatgtatcctcctccttacctagacaa | |
| | tgagaagagcaatggaaccattatccatgtgaaagggaaacacctttgt | |
| | ccaagtcccctatttcccggaccttctaagcccttttgggtgctggtggtg | |
| | gttgggggagtcctggcttgctatagcttgctagtaacagtggcctttatt | |
| | attttctgggtgaggagtaagaggagcaggctcctgcacagtgactaca | |
| | tgaacatgactccccgccgccccgggcccacccgcaagcattaccagcc | |
| | ctatgccccaccacgcgacttcgcagcctatcgctccagagtgaagttca | |
| | gcaggagcgcagacgcccccgcgtaccagcagggccagaaccagctct | |
| | ataacgagctcaatctaggacgaagagaggagtacgatgttttggaca | |
| | agagacgtggccgggaccctgagatggggggaaagccgagaaggaag | |
| | aaccctcaggaaggcctgtacaatgaactgcagaaagataagatggcg | |
| | gaggcctacagtgagattgggatgaaaggcgagcgccggaggggcaa | |
| | ggggcacgatggcctttaccagggtctcagtacagccaccaaggacacc | |
| | tacgacgcccttcacatgcaggccctgccccctcgc | |
| 36 | MLLLVTSLLLCELPHPAFLLIPDIQMTQTTSSLSASLGDRVT | Axicabtagene |
| | ISCRASQDISKYLNWYQQKPDGTVKLLIYHTSRLHSGVPSR | ciloleucel CD19 CAR |
| | FSGSGSGTDYSLTISNLEQEDIATYFCQQGNTLPYTFGGGT | amino acid |
| | KLEITGSTSGSGKPGSGEGSTKGEVKLQESGPGLVAPSQSL | sequence |
| | SVTCTVSGVSLPDYGVSWIRQPPRKGLEWLGVIWGSETT | |
| | YYNSALKSRLTIIKDNSKSQVFLKMNSLQTDDTAIYYCAKH | |
| | YYYGGSYAMDYWGQGTSVTVSSAAAIEVMYPPPYLDNE | |
| | KSNGTIIHVKGKHLCPSPLFPGPSKPFWVLVVVGGVLACY | |
| | SLLVTVAFIIFWVRSKRSRLLHSDYMNMTPRRPGPTRKHY | |
| | QPYAPPRDFAAYRSRVKFSRSADAPAYQQGQNQLYNEL | |
| | NLGRREEYDVLDKRRGRDPEMGGKPRRKNPQEGLYNEL | |
| | QKDKMAEAYSEIGMKGERRRGKGHDGLYQGLSTATKDT | |
| | YDALHMQALPPR | |
| 19 | DIQMTQTTSSLSASLGDRVTISCRASQDISKYLNWYQQKP | Axicabtagene |
| | DGTVKLLIYHTSRLHSGVPSRFSGSGSGTDYSLTISNLEQED | ciloleucel CD19 CAR |
| | IATYFCQQGNTLPYTFGGGTKLEITGSTSGSGKPGSGEGST | scFv amino acid |
| | KGEVKLQESGPGLVAPSQSLSVTCTVSGVSLPDYGVSWIR | sequence |
| | QPPRKGLEWLGVIWGSETTYYNSALKSRLTIIKDNSKSQVF | |
| | LKMNSLQTDDTAIYYCAKHYYYGGSYAMDYWGQGTSVT | |
| | VSS | |
| 25 | EVKLQESGPGLVAPSQSLSVTCTVSGVSLPDYGVSWIRQP | Axicabtagene |
| | PRKGLEWLGVIWGSETTYYNSALKSRLTIIKDNSKSQVFLK | ciloleucel CD19 CAR |
| | MNSLQTDDTAIYYCAKHYYYGGSYAMDYWGQGTSVTVS | HC Variable Region |
| | S | amino acid |
| | | sequence |
| 26 | GVSLPDYG | Axicabtagene |
| | | ciloleucel CD19 CAR |
| | | HC CDR1 amino acid |
| | | sequence |
| 27 | IWGSETT | Axicabtagene |
| | | ciloleucel CD19 CAR |
| | | HC CDR2 amino acid |
| | | sequence |
| 28 | AKHYYYGGSYAMDY | Axicabtagene |
| | | ciloleucel CD19 CAR |
| | | HC CDR3 amino acid |
| | | sequence |
| 20 | DIQMTQTTSSLSASLGDRVTISCRASQDISKYLNWYQQKP | Axicabtagene |
| | DGTVKLLIYHTSRLHSGVPSRFSGSGSGTDYSLTISNLEQED | ciloleucel CD19 CAR |
| | IATYFCQQGNTLPYTFGGGTKLEIT | LC Variable Region |
| | | amino acid |
| | | sequence |
| 21 | QDISKY | Axicabtagene |
| | | ciloleucel CD19 CAR |
| | | LC CDR1 amino acid |
| | | sequence |
| 22 | HTS | Axicabtagene |
| | | ciloleucel CD19 CAR |
| | | LC CDR2 amino acid |
| | | sequence |
| 23 | QQGNTLPYT | Axicabtagene |
| | | ciloleucel CD19 CAR |
| | | LC CDR3 amino acid |
| | | sequence |

TABLE 9
Annotation of tisagenlecleucel CD19 CAR sequences

| | Nucleotide | Amino Acid |
|---|---|---|
| Feature | Sequence Position | Sequence Position |
| CD8α signal peptide | 1-63 | 1-21 |
| FMC63 scFv | 64-789 | 22-263 |
| (V_{L}-3xG₄S linker-V_{H}) | | |
| CD8α hinge domain | 790-924 | 264-308 |
| CD8α transmembrane domain | 925-996 | 309-332 |
| 4-1BB costimulatory domain | 997-1122 | 333-374 |
| CD3ζ signaling domain | 1123-1458 | 375-486 |

TABLE 10
Annotation of lisocabtagene maraleucel CD19 CAR sequences

| | Nucleotide | Amino Acid |
|---|---|---|
| Feature | Sequence Position | Sequence Position |
| GMCSFR-α signal peptide | 1-66 | 1-22 |
| FMC63 scFv | 67-801 | 23-267 |
| (V_{L}-Whitlow linker-V_{H}) | | |
| IgG4 hinge domain | 802-837 | 268-279 |
| CD28 transmembrane domain | 838-921 | 280-307 |
| 4-1BB costimulatory domain | 922-1047 | 308-349 |
| CD3ζ signaling domain | 1048-1383 | 350-461 |

TABLE 11
Annotation of axicabtagene ciloleucel CD19 CAR sequences

| | Nucleotide | Amino Acid |
|---|---|---|
| Feature | Sequence Position | Sequence Position |
| CSF2RA signal peptide | 1-66 | 1-22 |
| FMC63 scFv | 67-801 | 23-267 |
| (V_{L}-Whitlow linker-V_{H}) | | |
| CD28 hinge domain | 802-927 | 268-309 |
| CD28 transmembrane domain | 928-1008 | 310-336 |
| CD28 costimulatory domain | 1009-1131 | 337-377 |
| CD3ζ signaling domain | 1132-1467 | 378-489 |

In some embodiments, a polynucleotide provided herein comprises a nucleotide sequence encoding a CD19 CAR, a variable domain of a CD19 CAR, or a set of CDRs (HCDR 1, 2, and 3 and LCDR 1, 2, and 3) that encodes a CD19 CAR as set forth in TABLE 12 below or a variable domain of a CD19 CAR or a set of CDRs (HCDR 1, 2, and 3 and LCDR 1, 2, and 3) therefrom. In some embodiments, a polynucleotide provided herein comprises a nucleotide sequence encoding a CD19 CAR, a variable domain of a CD19 CAR, or a set of CDRs (HCDR 1, 2, and 3 and LCDR 1, 2, and 3) that having at least 80% (e.g., at least 80%, at least 85%, at least 90%, at least 95%, at least 96%, at least 97%, at least 98%, at least 99%, or 100% identical) to a CD19 CAR as set forth in TABLE 12 below or a variable domain of a CD19 CAR or a set of CDRs (HCDR 1, 2, and 3 and LCDR 1, 2, and 3) therefrom, respectively.

TABLE 12
Exemplary CD19 antigen binding domains

| Antibody Name | Patents | Publications | Company |
|---|---|---|---|
| Abclon patent | 2018 WO2019125070 LEE, Jong | | Abclon |
| anti-CD19 | Seo, KI . . . ANTIBODY OR | | |
| | ANTIGEN-BINDING FRAGMENT | | |
| | THEREOF THAT SPECIFICALLY | | |
| | RECOGNIZES B CELL | | |
| | MALIGNANCIES, CHIMERIC | | |
| | ANTIGEN RECEPTOR | | |
| | COMPRISING SAME, AND USES | | |
| | THEREOF | | |
| | 2018 US20210061907 LEE, Jong | | |
| | Seo; KI . . . ANTIBODY OR | | |
| | ANTIGEN-BINDING FRAGMENT | | |
| | THEREOF THAT SPECIFICALLY | | |
| | RECOGNIZES B CELL | | |
| | MALIGNANCIES, CHIMERIC | | |
| | ANTIGEN RECEPTOR | | |
| | COMPRISING SAME, AND USES | | |
| | THEREOF | | |
| | 2018 WO2019112347 LEE, Jong | | |
| | Seo, KI . . . ANTIBODY OR | | |
| | ANTIGEN BINDING FRAGMENT | | |
| | THEREOF FOR SPECIFICALLY | | |
| | RECOGNIZING B CELL | | |
| | MALIGNANCY, CHIMERIC | | |
| | ANTIGEN RECEPTOR | | |
| | COMPRISING SAME, AND USE | | |
| | THEREOF | | |
| | 2018 US20200384023 LEE, Jong | | |
| | Seo; KI . . . Antibody Or Antigen | | |
| | Binding Fragment Thereof For | | |
| | Specifically Recognizing B Cell | | |
| | Malignancy, Chimeric Antigen | | |
| | Receptor Comprising Same And | | |
| | Use Thereof | | |
| | 2018 U.S. Pat. No. 11,534,462 Lee, Jong | | |
| | Seo; Ki . . . Antibody or antigen | | |
| | binding fragment thereof for | | |
| | specifically recognizing B cell | | |
| | malignancy, chimeric antigen | | |
| | receptor comprising same and | | |
| | use thereof | | |
| ALLO-501 | | 2020 NCT04416984 Phase 1/Phase | Allogene |
| | | 2 Safety and Efficacy of ALLO-501A | |
| | | Anti-CD19 Allogeneic CAR T Cells in | |
| | | Adults With Relapsed/Refractory | |
| | | Large B Cell Lymphoma (ALPHA-2) | |
| Baylor Coll. Med. | | 2014 NCT02050347 Phase 1 | Baylor Coll. |
| anti-CD19 CAR | | Activated T Lymphocytes Expressing | Med. |
| | | CARs, Relapsed CD19+ Malignancies | |
| | | Post-Allo HSCT(CARPASCIO) | |
| Beijing Cancer | | 2014 NCT02247609 Phase 1/Phase | Beijing Cancer |
| Hosp. anti-CD19 | | 2 Evaluation of 4th Generation | Hosp. |
| CAR 4th Gen. | | Safety-designed CAR T Cells | |
| | | Targeting High-risk and Refractory B | |
| | | Cell Lymphomas | |
| Beijing Marino Bio | 2020 US20210196756 YAN, | | Beijing Marino |
| patent anti-CD19 | Yongchao; Zh . . . ANTI-CD19 | | Bio |
| CAR | CAR-T CELL | | |
| Beijing Meikang | 2019 WO2020108644 LI, | | Beijing Meikang |
| Geno-immune | Junfang CD19-AND CD22- | | Geno-immune |
| patent anti-CD19 | BASED COMBINED CAR-T | | |
| CAR | IMMUNOTHERAPY | | |
| | 2019 WO2020108646 ZHANG, | | |
| | Rui CD19-AND PSMA-BASED | | |
| | COMBINED CAR-T | | |
| | IMMUNOTHERAPY | | |
| | 2019 WO2020108645 GUI, | | |
| | Siqian CD19-AND BCMA-BASED | | |
| | COMBINED CAR-T | | |
| | IMMUNOTHERAPY | | |
| | 2019 WO2020108643 WANG, | | |
| | Jiaxing CD19-AND CD70-BASED | | |
| | COMBINED CAR-T | | |
| | IMMUNOTHERAPY | | |
| | 2019 WO2020108642 LI, | | |
| | Yuchen CD19-AND CD30-BASED | | |
| | COMBINED CAR-T | | |
| | IMMUNOTHERAPY | | |
| | 2019 WO2019161796 ZHANG, | | |
| | Rui A CD19-BASED CHIMERIC | | |
| | ANTIGEN RECEPTOR AND | | |
| | APPLICATION THEREOF | | |
| Bioswan patent | 2019 WO2020125653 DA, Liang | | Bioswan |
| anti-CD19 | MONOCLONAL ANTIBODY | | |
| | WHICH FIGHTS CD19 AND | | |
| | APPLICATION THEREOF | | |
| Cancer Res. Tech. | 2018 US20190241671 Super, | | Cancer Research |
| patent anti-CD19 | Michael; D . . . ANTI-CD19 | | Technology |
| | ANTIBODIES WITH REDUCED | | |
| | IMMUNOGENICITY | | |
| | 2018 U.S. Pat. No. 11,208,496 Super, | | |
| | Michael (L . . . Anti-CD19 | | |
| | antibodies with reduced | | |
| | immunogenicity | | |
| Carsgen patent | 2017 WO2018108106 WANG, | | Carsgen |
| anti-CD19 | Peng, [symbol] , G . . . ANTI-CD19 | | |
| | HUMANIZED ANTIBODY AND | | |
| | IMMUNE EFFECTOR CELL | | |
| | TARGETING CD19 | | |
| | 2017 US20200062843 WANG, | | |
| | Peng; GAO, . . . ANTI-CD19 | | |
| | HUMANIZED ANTIBODY AND | | |
| | IMMUNE EFFECTOR CELL | | |
| | TARGETING CD19 | | |
| | 2017 U.S. Pat. No. 11,427,633 Wang, Peng | | |
| | (Shang . . . Anti-CD19 humanized | | |
| | antibody and immune effector | | |
| | cell targeting cd 19 | | |
| Chinese PLA | | 2014 NCT02081937 Phase 1/Phase | Chinese PLA |
| Gen. Hosp. anti- | | 2 CART-19 Immunotherapy in | Gen. Hosp. |
| CD19 CAR | | Mantle Cell Lymphoma | |
| | | 2013 NCT01864889 N/A Treatment | |
| | | of Relapsed and/or Chemotherapy | |
| | | Refractory B-cell Malignancy by | |
| | | CART19 | |
| City of Hope | 2021 WO2022125837 BROWN, | | City of Hope |
| patent anti-CD19 | Christine E. COMPOSITIONS | | |
| CAR | AND USES OF CD19 TARGETED | | |
| | CHIMERIC ANTIGEN RECEPTOR | | |
| | MODIFIED IMMUNE CELLS | | |
| | 2018 WO2019094498 WANG, | | |
| | Xiuli, FORM . . . TREATMENT OF | | |
| | CNS LYMPHOMA AND | | |
| | SYSTEMIC LYMPHOMA WITH | | |
| | INTRACEREBROVENTRICULARLY | | |
| | ADMINISTERED CD19 CAR | | |
| coltuximab | 2018 WO2018183494 HICKS, | 2011 NCT01470456 Phase 2 | ImmunoGen |
| ravtansine | Stuart, Wi . . . CD19-TARGETING | Combination of SAR3419 and | Sanofi |
| | ANTIBODY-DRUG CONJUGATES | Rituximab in Relapsed/Refractory | |
| | 2016 US20170196988 Morariu, | Diffuse Large B-Cell Lymphoma | |
| | Rodica; USE OF ANTI-CD19 | 2011 NCT01472887 Phase 2 | |
| | MAYTANSINOID | SAR3419 as Single Agent in | |
| | IMMUNOCONJUGATE | Relapsed-Refractory Diffuse Large | |
| | ANTIBODY FOR THE | B-Cell Lymphoma (DLBCL) Patients | |
| | TREATMENT OF B-CELL | 2011 NCT01440179 Phase 2 | |
| | MALIGNANCIES SYMPTOMS | SAR3419 in Acute Lymphoblastic | |
| | 2016 WO2016180941 BARBOT, | Leukemia | |
| | Anne, BOU . . . LIQUID | 2008 NCT00796731 Phase 1 | |
| | COMPOSITIONS FOR ANTI- | SAR3419 Administered Weekly in | |
| | CD19 ANTIBODY-DRUG | Patients With Relapsed/Refractory | |
| | CONJUGATES | CD19-positive B-cell Non-Hodgkin's | |
| | 2013 WO2014058947 CAI, Ti, | Lymphoma | |
| | ZAKS, Ta . . . COMPOSITIONS | 2007 NCT00539682 Phase 1 | |
| | AND METHODS FOR TREATING | SAR3419 in Patients With Relapsed | |
| | CANCER USING PI3K INHIBITOR | or Refractory B-Cell Non-Hodgkin's | |
| | AND ANTI-CD19 | Lymphoma (NHL) | |
| | MAYTANSINOID | 2007 NCT00549185 Phase 1 Multi- | |
| | IMMUNOCONJUGATE | dose-escalation Safety and | |
| | 2012 WO2013017540 BESRET, | Pharmacokinetic Study of SAR3419 | |
| | Laurent, . . . COMBINATION | as Single Agent in | |
| | THERAPY FOR THE TREATMENT | Relapsed/Refractory B-cell Non | |
| | OF CD19 B-CELL | Hodgkin's Lymphoma | |
| | MALIGNANCIES SYMPTOMS | 2018 Trněný M, Verhoef . . . A Phase | |
| | COMPRISING AN ANTI-CD19 | 2 multicenter study of the anti- | |
| | MAYTANSINOID | CD19 antibody drug conjugate | |
| | IMMUNOCONJUGATE AND | coltuximab ravtansine (SAR3419) in | |
| | RITUXIMAB | patients with relapsed or refractory | |
| | 2012 WO2012156455 | diffuse large B-cell lymphoma | |
| | MORARIU, Rodica USE OF | previously treated with rituximab- | |
| | ANTI-CD19 MAYTANSINOID | based immunotherapy. | |
| | IMMUNOCONJUGATE | 2017 Ereño-Orbea J, Si . . . Structural | |
| | ANTIBODY FOR THE | Basis of Enhanced Crystallizability | |
| | TREATMENT OF B-CELL | Induced by a Molecular Chaperone | |
| | MALIGNANCIES SYMPTOMS | for Antibody Antigen-Binding | |
| | 2012 US20140072587 Morariu, | Fragments. | |
| | Rodica; USE OF ANTI-CD19 | 2017 Bouillon-Pichault . . . | |
| | MAYTANSINOID | Translational Model-Based Strategy | |
| | IMMUNOCONJUGATE | to Guide the Choice of Clinical | |
| | ANTIBODY FOR THE | Doses for Antibody-Drug | |
| | TREATMENT OF B-CELL | Conjugates. | |
| | MALIGNANCIES SYMPTOMS | 2016 Coiffier B, Thieb . . . A phase II, | |
| | 2012 U.S. Pat. No. 9,555,126 Morariu, | single-arm, multicentre study of | |
| | Rodica Use of anti-CD19 | coltuximab ravtansine (SAR3419) | |
| | maytansinoid | and rituximab in patients with | |
| | immunoconjugate antibody for | relapsed or refractory diffuse large | |
| | the treatment of B-cell | B-cell lymphoma. | |
| | malignancies symptoms | 2015 Kantarjian H M, Li . . . A Phase II | |
| | | Study of Coltuximab Ravtansine | |
| | | (SAR3419) Monotherapy in Patients | |
| | | With Relapsed or Refractory Acute | |
| | | Lymphoblastic Leukemia. | |
| | | 2015 Hong E J, Erickson . . . Design of | |
| | | Coltuximab Ravtansine, a CD19- | |
| | | Targeting Antibody-Drug Conjugate | |
| | | (ADC) for the Treatment of B-cell | |
| | | Malignancies: Structure-Activity | |
| | | Relationships and Preclinical | |
| | | Evaluation. | |
| | | 2013 Ribrag V, Dupuis . . . A PHASE I | |
| | | STUDY OF SAR3419, AN ANTI-CD19 | |
| | | ANTIBODY MAYTANSINOID | |
| | | CONJUGATE, ADMINISTERED ONCE | |
| | | WEEKLY IN PATIENTS WITH | |
| | | RELAPSED/REFRACTORY B-CELL | |
| | | NON HODGKIN'S LYMPHOMA. | |
| | | 2012 Younes A, Kim S, . . . Phase I | |
| | | multidose-escalation study of the | |
| | | anti-CD19 maytansinoid | |
| | | immunoconjugate SAR3419 | |
| | | administered by intravenous | |
| | | infusion every 3 weeks to patients | |
| | | with relapsed/refractory B-cell | |
| | | lymphoma. | |
| | | 2011 Blanc V, Bousseau . . . SAR3419: | |
| | | an anti-CD19-Maytansinoid | |
| | | Immunoconjugate for the | |
| | | treatment of B-cell malignancies. | |
| | | 2009 Al-Katib A M, Abou . . . Superior | |
| | | antitumor activity of SAR3419 to | |
| | | rituximab in xenograft models for | |
| | | non-Hodgkin's lymphoma. | |
| | | 1994 Roguska M A, Peder . . . | |
| | | Humanization of murine | |
| | | monoclonal antibodies through | |
| | | variable domain resurfacing. | |
| | | ASCO 2011 Phase I/II study of the | |
| | | anti-CD19 maytansinoid | |
| | | immunoconjugate SAR3419 | |
| | | administered weekly to patients | |
| | | (pts) with relapsed/refractory B-cell | |
| | | non-Hodgkin lymphoma (NHL). B. | |
| | | Coiffier | |
| | | ASCO 2012 Phase I study cohort | |
| | | evaluating an optimized | |
| | | administration schedule of | |
| | | SAR3419, an anti-CD19 DM4-loaded | |
| | | antibody drug conjugate (ADC), in | |
| | | patients (pts) with CD19 positive | |
| | | relapsed/refractory b-cell non- | |
| | | Hodgkin's lymphoma | |
| | | (NCT00796731) Bertrand Coiffier . . . | |
| | | ASCO 2014 Starlyte phase II study | |
| | | of coltuximab ravtansine (CoR, | |
| | | SAR3419) single agent: Clinical | |
| | | activity and safety in patients (pts) | |
| | | with relapsed/refractory (R/R) | |
| | | diffuse large B-cell lymphoma | |
| | | (DLBCL; NCT01472887) Marek | |
| | | Trneny, Gre . . . | |
| | | ASH 2013 Phase II Study Of Anti- | |
| | | CD19 Antibody Drug Conjugate | |
| | | (SAR3419) In Combination With | |
| | | Rituximab: Clinical Activity and | |
| | | Safety In Patients With | |
| | | Relapsed/Refractory Diffuse Large | |
| | | B-Cell Lymphoma (NCT01470456) | |
| | | Bertrand Coiffier . . . | |
| denintuzumab | 2019 US20190381172 | 2016 NCT02855359 Phase 2 | Seattle Genetics |
| mafodotin | Amsberry, Kent; J . . . STABLE | Denintuzumab Mafodotin (SGN- | |
| | FORMULATIONS FOR ANTI- | CD19A) Combined With RCHOP or | |
| | CD19 ANTIBODIES AND | RCHP Versus RCHOP Alone in | |
| | ANTIBODY-DRUG CONJUGATES | Diffuse Large B-Cell Lymphoma or | |
| | 2017 WO2018017928 KOSTIC, | Follicular Lymphoma | |
| | Ana COMBINATION THERAPY | 2015 NCT02592876 Phase 2 | |
| | USING A CD19-ADC AND RCHP | Treatment Study of Denintuzumab | |
| | 2017 US20200230254 Kostic, | Mafodotin (SGN-CD19A) Plus RICE | |
| | Ana; COMBINATION THERAPY | Versus RICE Alone for Diffuse Large | |
| | USING A CD19-ADC AND RCHP | B-Cell Lymphoma | |
| | 2016 WO2016130902 LAW, | 2013 NCT01786135 Phase 1 A | |
| | Che-Leung, S . . . COMBINATION | Safety Study of SGN-CD19A for B- | |
| | THERAPY USING A CD19-ADC | Cell Lymphoma | |
| | AND VINCRISTINE | 2013 NCT01786096 Phase 1 A | |
| | 2015 WO2015157297 ZHAO, | Safety Study of SGN-CD19A for | |
| | Baiteng, AL . . . OPTIMAL DOSING | Leukemia and Lymphoma | |
| | OF A CD19-ANTIBODY DRUG | 2019 Jones L, McCalmon . . . | |
| | CONJUGATE | Preclinical activity of the antibody- | |
| | 2015 WO2015157286 | drug conjugate denintuzumab | |
| | AMSBERRY, Kent, J . . . STABLE | mafodotin (SGN-CD19A) against | |
| | FORMULATIONS FOR ANTI- | pediatric acute lymphoblastic | |
| | CD19 ANTIBODIES AND | leukemia xenografts. | |
| | ANTIBODY-DRUG CONJUGATES | ASCO 2014 Interim analysis of a | |
| | 2015 US20170028062 | phase 1, open-label, dose- | |
| | Amsberry, Kent; J . . . STABLE | escalation study of SGN-CD19A in | |
| | FORMULATIONS FOR ANTI- | patients with relapsed or refractory | |
| | CD19 ANTIBODIES AND | B-lineage non-Hodgkin lymphoma | |
| | ANTIBODY-DRUG CONJUGATES | (NHL) Andres Forero-Tor . . . | |
| | 2015 US20170182178 ZHAO, | ASCO 2016 A randomized, phase 2 | |
| | Baiteng; AL . . . OPTIMAL DOSING | trial of denintuzumab mafodotin | |
| | OF A CD19-ANTIBODY DRUG | and RICE vs RICE alone in the | |
| | CONJUGATE | treatment of patients (pts) with | |
| | 2012 US20120294853 | relapsed/refractory (r/r) diffuse | |
| | McDonagh, Charlot . . . CD19 | large B-cell lymphoma (DLBCL) who | |
| | Binding Agents and Uses | are candidates for autologous stem | |
| | Thereof | cell transplant (ASCT) Robert W. | |
| | 2011 US20110312088 | Chen, E . . . | |
| | McDonagh, Charlot . . . CD19 | ASH 2013 A First-In-Human Phase 1 | |
| | BINDING AGENTS AND USES | Study Of The Antibody-Drug | |
| | THEREOF | Conjugate SGN-CD19A In Relapsed | |
| | 2011 U.S. Pat. No. 8,242,252 McDonagh, | Or Refractory B-Lineage Acute | |
| | Charlot . . . CD19 binding agents | Leukemia and Highly Aggressive | |
| | and uses thereof | Lymphoma Uma Borate, MD, A . . . | |
| | 2008 WO2009052431 | ASH 2014 Interim Analysis of a | |
| | MCDONAGH, Charlot . . . CD19 | Phase 1 Study of the Antibody-Drug | |
| | BINDING AGENTS AND USES | Conjugate SGN-CD19A in Relapsed | |
| | THEREOF | or Refractory B-Lineage Acute | |
| | 2008 U.S. Pat. No. 7,968,687 McDonagh, | Leukemia and Highly Aggressive | |
| | Charlot . . . CD19 binding agents | Lymphoma Amir T Fathi, Rob . . . | |
| | and uses thereof | ASH 2015 A Phase 1 Study of | |
| | 2008 US20090136526 | Denintuzumab Mafodotin (SGN- | |
| | McDonagh, Charlot . . . CD19 | CD19A) in Adults with Relapsed or | |
| | Binding Agents and Uses | Refractory B-Lineage Acute | |
| | Thereof | Leukemia (B-ALL) and Highly | |
| | | Aggressive Lymphoma Amir T Fathi, | |
| | | MD, . . . | |
| | | ASH 2016 Denintuzumab Mafodotin | |
| | | Stimulates Immune Responses and | |
| | | Synergizes with CD20 Antibodies to | |
| | | Heighten Anti-Tumor Activity in | |
| | | Preclinical Models of Non-Hodgkin | |
| | | Lymphoma Heather A. Van Ep . . . | |
| Duke U. patent | 2013 US20140056896 Tedder, | | Duke U. |
| anti-CD19 | Thomas F . . . ANTI-CD19 | | |
| | ANTIBODIES AND USES IN B | | |
| | CELL DISORDERS | | |
| | 2013 U.S. Pat. No. 9,260,530 Tedder, | | |
| | Thomas F . . . Anti-CD19 | | |
| | antibodies and uses in B cell | | |
| | disorders | | |
| | 2012 US20130084294 Tedder, | | |
| | Thomas F . . . ANTI-CD19 | | |
| | ANTIBODIES AND USES IN B | | |
| | CELL DISORDERS | | |
| | 2010 U.S. Pat. No. 8,444,973 Tedder, | | |
| | Thomas F . . . Anti-CD19 | | |
| | antibodies and uses in B cell | | |
| | disorders | | |
| | 2008 US20100158901 Tedder, | | |
| | Thomas F . . . ANTI-CD19 | | |
| | ANTIBODY THERAPY FOR | | |
| | AUTOIMMUNE DISEASE | | |
| | 2008 US20090246195 Tedder, | | |
| | Thomas F.; ANTI-CD19 | | |
| | ANTIBODY THERAPY FOR | | |
| | TRANSPLANTATION | | |
| | 2006 WO2006133450 TEDDER, | | |
| | Thomas, F.; ANTI-CD19 | | |
| | ANTIBODY THERAPY FOR THE | | |
| | TRANSPLANTATION | | |
| | 2006 US20060280738 Tedder, | | |
| | Thomas F.; Anti-CD19 antibody | | |
| | therapy for transplantation | | |
| | 2006 WO2006121852 TEDDER, | | |
| | Thomas, F . . . ANTI-CD19 | | |
| | ANTIBODY THERAPY FOR | | |
| | AUTOIMMUNE DISEASE | | |
| | 2006 US20060263357 Tedder, | | |
| | Thomas F . . . Anti-CD19 | | |
| | antibody therapy for | | |
| | autoimmune disease | | |
| | 2006 US20060233791 Tedder, | | |
| | Thomas F . . . Anti-CD19 | | |
| | antibodies and uses in | | |
| | oncology | | |
| Elpis Bio patent | 2020 WO2021034952 CHEN, | | Elpis Bio |
| anti-CD19 | Yan, NGUYEN . . . ANTI-CD19 | | |
| | ANTIBODIES AND USES | | |
| | THEREOF | | |
| | 2020 US20220289843 CHEN, | | |
| | Yan; NGUYEN . . . ANTI-CD19 | | |
| | ANTIBODIES AND USES | | |
| | THEREOF | | |
| Eureka patent | 2019 US20190315859 LIU, | | Eureka |
| anti-CD19 | HONG; Lu, Ji . . . ANTIBODY | | |
| | AGENTS SPECIFIC FOR HUMAN | | |
| | CD19 AND USES THEREOF | | |
| | 2017 US20180134787 LIU, | | |
| | HONG; Lu, Ji . . . ANTIBODY | | |
| | AGENTS SPECIFIC FOR HUMAN | | |
| | CD19 AND USES THEREOF | | |
| | 2017 U.S. Pat. No. 10,301,388 Liu, Hong | | |
| | (Emeryv . . . Antibody agents | | |
| | specific for human CD19 and | | |
| | uses thereof | | |
| | 2017 U.S. Pat. No. 10,098,951 Lu, Jingwei | | |
| | (Unio . . . Antibody/T-cell | | |
| | receptor chimeric constructs | | |
| | and uses thereof | | |
| | 2016 WO2017066136 LIU, | | |
| | Hong, LU, Ji . . . ANTIBODY | | |
| | AGENTS SPECIFIC FOR HUMAN | | |
| | CD19 AND USES THEREOF | | |
| Feinstein Inst. | 2021 WO2022120107 | | Feinstein Inst. |
| patent anti-CD19 | VAISELBUH, Sarah, . . . CD19 | | |
| CAR | CHIMERIC ANTIGEN RECEPTOR- | | |
| | EXOSOME TARGETS AND | | |
| | INDUCES CYTOTOXICITY IN | | |
| | CD19 POSITIVE B-CELL | | |
| | MALIGNANCIES | | |
| Fuzhou Tcelltech | 2021 WO2021170146 HUANG, | | Fuzhou Tcelltech |
| patent anti-CD19 | Gangxiong PREPARATION OF | | |
| | NEW-TYPE ANTI-CD19 | | |
| | ANTIBODY AND CD19-CAR-T | | |
| | CELL, AND USE THEREOF | | |
| GBR 401 | 2014 US20140286934 BLEIN, | 2014 Breton C S, Nahima . . . A novel | Glenmark/Ichnos |
| | Stanislas; . . . HUMANIZED | anti-CD19 monoclonal antibody | |
| | ANTIBODIES THAT BIND TO | (GBR 401) with high killing activity | |
| | CD19 AND THEIR USES | against B cell malignancies. | |
| | 2010 US20100215651 BLEIN, | | |
| | STANISLAS; . . . Humanized | | |
| | antibodies that bind to CD19 | | |
| | and their uses | | |
| | 2010 WO2010095031 BLEIN, | | |
| | Stanislas; . . . HUMANIZED | | |
| | ANTIBODIES THAT BIND TO | | |
| | CD19 AND THEIR USES | | |
| | 2010 U.S. Pat. No. 8,679,492 Blein, | | |
| | Stanislas; . . . Humanized | | |
| | antibodies that bind to CD19 | | |
| | and their uses | | |
| Gracell patent | 2021 WO2021223720 ZHANG, | | Gracell Bio |
| anti-CD19 | Hua, SHEN, . . . HUMANIZED | | |
| | CD19 ANTIBODY AND USE | | |
| | THEREOF | | |
| Green Cross | 2019 WO2020055040 HER, | | Green Cross |
| patent anti-CD19 | Jung Hyun, J . . . | | Morphosys |
| | PHARMACEUTICAL | | |
| | COMBINATIONS FOR TREATING | | |
| | TUMOR COMPRISING ANTI- | | |
| | CD19 ANTIBODY AND NATURAL | | |
| | KILLER CELL | | |
| | 2019 US20220047632 HER, | | |
| | Jung Hyun; J . . . | | |
| | PHARMACEUTICAL | | |
| | COMBINATIONS FOR TREATING | | |
| | TUMOR COMPRISING ANTI- | | |
| | CD19 ANTIBODY AND NATURAL | | |
| | KILLER CELL | | |
| huB4-DGN462 | | 2019 Hicks S W, Tarante . . . The novel | ImmunoGen |
| | | CD19-targeting antibody-drug | |
| | | conjugate huB4-DGN462 shows | |
| | | improved anti-tumor activity than | |
| | | SAR3419 in CD19-positive | |
| | | lymphoma and leukemia models. | |
| iCarTAB anti-CD19 | | 2016 NCT02782351 Phase 1/Phase | iCarTAB Xuzhou |
| CAR | | 2 Humanized CAR-T Therapy for | Med. Coll. |
| | | Treatment of B Cell Malignancy | |
| | | 2022 Shi M, Li L, Wang . . . Safety and | |
| | | efficacy of a humanized CD19 | |
| | | chimeric antigen receptor T Cells for | |
| | | relapsed/refractory acute | |
| | | lymphoblastic leukaemia. | |
| | | 2019 Liu F, Zhang H, W . . . First-in- | |
| | | Human Trial of Bcma-CD19 | |
| | | Compound CAR with Remarkable | |
| | | Donor-Specific Antibody Reduction. | |
| IDD-Tech patent | 2018 WO2019011918 | | IDD-Tech |
| anti-CD19 | VERMOT-DESROCHES, . . . | | |
| | TREATMENT OF B CELL | | |
| | MALIGNANCIES USING | | |
| | AFUCOSYLATED PRO- | | |
| | APOPTOTIC ANTI-CD19 | | |
| | ANTIBODIES IN COMBINATION | | |
| | WITH ANTI CD20 ANTIBODIES | | |
| | OR CHEMOTHERAPEUTICS | | |
| | 2018 US20220227862 | | |
| | VERMOT-DESROCHES, . . . | | |
| | TREATMENT OF B CELL | | |
| | MALIGNANCIES USING | | |
| | AFUCOSYLATED PRO- | | |
| | APOPTOTIC ANTI-CD19 | | |
| | ANTIBODIES IN COMBINATION | | |
| | WITH ANTI CD20 ANTIBODIES | | |
| | OR CHEMOTHERAPEUTICS | | |
| | 2011 WO2012010602 | | |
| | VERMOT-DESROCHES, . . . | | |
| | METHOD TO IMPROVE | | |
| | GLYCOSYLATION PROFILE AND | | |
| | TO INDUCE MAXIMAL | | |
| | CYTOTOXICITY FOR ANTIBODY | | |
| | 2011 WO2012010562 | | |
| | VERMOT-DESROCHES, . . . ANTI- | | |
| | CD19 ANTIBODY HAVING ADCC | | |
| | AND CDC FUNCTIONS AND | | |
| | IMPROVED GLYCOSYLATION | | |
| | PROFILE | | |
| | 2011 WO2012010561 SALLES, | | |
| | Gilles; V . . . ANTI-CD19 | | |
| | ANTIBODY HAVING ADCC | | |
| | FUNCTION WITH IMPROVED | | |
| | GLYCOSYLATION PROFILE | | |
| | 2011 US20130183306 Salles, | | |
| | Gilles; V . . . ANTI-CD19 | | |
| | ANTIBODY HAVING ADCC | | |
| | FUNCTION WITH IMPROVED | | |
| | GLYCOSYLATION PROFILE | | |
| | 2011 US20130224190 Vermot- | | |
| | Desroches, . . . ANTI-CD19 | | |
| | ANTIBODY HAVING ADCC AND | | |
| | CDC FUNCTIONS AND | | |
| | IMPROVED GLYCOSYLATION | | |
| | PROFILE | | |
| | 2011 U.S. Pat. No. 9,120,856 Salles, Gilles; | | |
| | V . . . Anti-CD19 antibody having | | |
| | ADCC function with improved | | |
| | glycosylation profile | | |
| | 2011 U.S. Pat. No. 9,663,583 Vermot- | | |
| | Desroches, . . . Anti-CD19 | | |
| | antibody having ADCC and CDC | | |
| | functions and improved | | |
| | glycosylation profile | | |
| | USRE049192 Anti-CD19 | | |
| | antibody having ADCC and CDC | | |
| | functions and improved | | |
| | glycosylation profile | | |
| IKS03 | 2019 WO2019215510 SONG, | 2022 NCT05365659 Phase 1 IKS03 | Iksuda |
| | Ho, Young, . . . COMPOSITIONS | in Patients With Advanced B Cell | LegoChem |
| | AND METHODS RELATED TO | Non-Hodgkin Lymphomas | NovImmune |
| | ANTI-CD19 ANTIBODY DRUG | | |
| | CONJUGATES | | |
| | 2019 US20200095317 Song, Ho | | |
| | Young; P . . . COMPOSITIONS | | |
| | AND METHODS RELATED TO | | |
| | ANTI-CD19 ANTIBODY DRUG | | |
| | CONJUGATES | | |
| | 2017 WO2018083535 FISCHER, | | |
| | Nicolas ANTI-CD19 ANTIBODIES | | |
| | AND METHODS OF USE | | |
| | THEREOF | | |
| | 2017 US20180142018 Fischer, | | |
| | Nicolas; ANTI-CD19 | | |
| | ANTIBODIES AND METHODS OF | | |
| | USE THEREOF | | |
| Immunomedics | 2017 US20170152315 Hansen, | | Immunomedics |
| hA19 | Hans J.; . . . Anti-CD19 | | |
| | Antibodies | | |
| | 2016 US20160319020 Hansen, | | |
| | Hans J.; . . . Anti-CD19 | | |
| | Antibodies | | |
| | 2016 U.S. Pat. No. 9,605,071 Hansen, Hans | | |
| | J.; . . . Anti-CD19 antibodies | | |
| | 2015 US20150252110 Hansen, | | |
| | Hans J.; . . . Anti-CD19 | | |
| | Antibodies | | |
| | 2013 US20140112865 Hansen, | | |
| | Hans J.; . . . Anti-CD19 | | |
| | Antibodies | | |
| | 2013 U.S. Pat. No. 9,056,917 Hansen, Hans | | |
| | J.; . . . Anti-CD19 antibodies | | |
| | 2013 U.S. Pat. No. 8,624,001 Hansen, Hans | | |
| | J.; . . . Anti-CD19 antibodies | | |
| | 2013 US20140017197 Hansen, | | |
| | Hans J.; . . . Anti-CD19 | | |
| | Antibodies | | |
| | 2012 US20130121913 Hansen, | | |
| | Hans J.; . . . Anti-CD19 | | |
| | Antibodies | | |
| | 2012 U.S. Pat. No. 8,486,395 Hansen, Hans | | |
| | J.; . . . Anti-CD19 antibodies | | |
| | 2012 US20120189542 Hansen, | | |
| | Hans J.; . . . Anti-CD19 | | |
| | Antibodies | | |
| | 2012 U.S. Pat. No. 8,337,840 Hansen, Hans | | |
| | J.; . . . Anti-CD19 antibodies | | |
| | 2010 US20110052489 Hansen, | | |
| | Hans J.; . . . Anti-CD19 | | |
| | Antibodies | | |
| | 2010 U.S. Pat. No. 8,147,831 Hansen, Hans | | |
| | J.; . . . Anti-CD19 antibodies | | |
| | 2009 WO2010053716 HANSEN, | | |
| | Hans J.; . . . IMPROVED ANTI- | | |
| | CD19 ANTIBODIES | | |
| | 2008 U.S. Pat. No. 7,902,338 Hansen, Hans | | |
| | J.; . . . Anti-CD19 antibodies | | |
| | 2008 US20100068136 Hansen, | | |
| | Hans J.; . . . Anti-CD19 | | |
| | Antibodies | | |
| | 2006 U.S. Pat. No. 7,462,352 Hansen, Hans | | |
| | J.; . . . Methods of treatng B-cell | | |
| | diseases using humanized anti- | | |
| | CD19 antibodies | | |
| | 2004 WO2005012493 HANSEN, | | |
| | Hans J.; . . . ANTI-CD19 | | |
| | ANTIBODIES | | |
| Indian Inst. Tech. | 2019 WO2019159193 | | Indian Inst. |
| patent anti-CD19 | PURWAR, Rahul, DW . . . NOVEL | | Tech. Bombay |
| CAR | HUMANIZED ANTI-CD19 | | |
| | CHIMERIC ANTIGEN RECEPTOR, | | |
| | ITS NUCELIC ACID SEQUENCE | | |
| | AND ITS PREPARATION | | |
| inebilizumab-cdon | 2022 WO2022236047 KATZ, | 2020 NCT04540497 Phase 3 A Study | Medimmune |
| | Eliezer USE OF AN ANTI-CD19 | of Inebilizumab Efficacy and Safety | Viela Bio |
| | ANTIBODY TO TREAT | in IgG4- Related Disease | |
| | MYASTHENIA GRAVIS | 2019 NCT04174677 Phase 2 Safety | |
| | 2021 WO2022094334 SHE, | and Tolerability of Inebilizumab, | |
| | Dewei, RATCH . . . USE OF AN | VIB4920, or the Combination in | |
| | ANTI-CD19 ANTIBODY TO | Highly Sensitized Candidates | |
| | TREAT AUTOIMMUNE DISEASE | Awaiting Kidney Transplantation | |
| | 2020 US20210061906 | From a Deceased Donor | |
| | Damschroder, Meli . . . | 2014 NCT02271945 Phase 1/Phase | |
| | HUMANIZED ANTI-CD19 | 2 Safety/Efficacy of MEDI-551 in | |
| | ANTIBODIES AND THEIR USE IN | Combination With | |
| | TREATMENT OF ONCOLOGY, | Immunomodulating Therapies in | |
| | TRANSPLANTATION AND | Subjects With Aggressive B-cell | |
| | AUTOIMMUNE DISEASE | Lymphomas | |
| | 2020 WO2020219743 KATZ, | 2014 NCT02200770 Phase 2/Phase | |
| | Eliezer, DR . . . USE OF AN ANTI- | 3 A Double-masked, Placebo- | |
| | CD19 ANTIBODY TO TREAT | controlled Study With Open Label | |
| | AUTOIMMUNE DISEASE | Period to Evaluate MEDI-551 in | |
| | 2020 US20220204617 KATZ, | Neuromyelitis Optica and | |
| | Eliezer; DR . . . USE OF AN ANTI- | Neuromyelitis Optica Spectrum | |
| | CD19 ANTIBODY TO TREAT | Disorders | |
| | AUTOIMMUNE DISEASE | 2013 NCT01861340 Phase 0 | |
| | 2018 US20180273621 | Lenalidomide, Dexamethasone and | |
| | Damschroder, Meli . . . | MEDI-551 in Untreated Multiple | |
| | HUMANIZED ANTI-CD19 | Myeloma | |
| | ANTIBODIES AND THEIR USE IN | 2012 NCT01585766 Phase 1/Phase | |
| | TREATMENT OF ONCOLOGY, | 2 Safety and Efficacy Study of MEDI- | |
| | TRANSPLANTATION AND | 551, a B-cell Depleting Agent, to | |
| | AUTOIMMUNE DISEASE | Treat Multiple Sclerosis | |
| | 2016 US20170088628 Yao, | 2012 NCT01466153 Phase 2 A | |
| | Yihong; Stre . . . Compositions | Phase 2, Multicenter, Open-label | |
| | and Methods for Identifying B | Study of MEDI-551 in Adults With | |
| | Cell Malignancies Responsive | Relapsed or Refractory Chronic | |
| | to B Cell Depleting Therapy | Lymphocytic Leukemia (CLL) | |
| | 2015 US20150252431 YAO, | 2011 NCT01453205 Phase 2 MEDI- | |
| | YIHONG; STRE . . . | 551 in Adults With Relapsed or | |
| | COMPOSITIONS AND | Refractory Diffuse Large B-Cell | |
| | METHODS FOR IDENTIFYING B | Lymphoma (DLBCL) | |
| | CELL MALIGNANCIES | 2011 NCT01377116 Phase 1 A | |
| | RESPONSIVE TO B CELL | Phase 1, Dose-escalation Study of | |
| | DEPLETING THERAPY | MEDI-551 in Japanese Adult | |
| | 2014 US20160145335 | Patients With Relapsed or | |
| | Damschroder, Meli . . . | Refractory Advanced B-cell | |
| | HUMANIZED ANTI-CD19 | Malignancies | |
| | ANTIBODIES AND THEIR USE IN | 2011 NCT01957579 Phase 1 A | |
| | TREATMENT OF ONCOLOGY, | Phase 1, Dose-escalation Study of | |
| | TRANSPLANTATION AND | MEDI-551 in Japanese Adult | |
| | AUTOIMMUNE DISEASE | Patients With Relapsed or | |
| | 2014 U.S. Pat. No. 9,896,505 Damschroder, | Refractory Advanced B-cell | |
| | Meli . . . Humanized anti-CD19 | Malignancies | |
| | antibodies and their use in | 2010 NCT00946699 Phase 1 A Study | |
| | treatment of oncology, | of the Safety and Tolerability of | |
| | transplantation and | MEDI-551 in Scleroderma | |
| | autoimmune disease | 2010 NCT00983619 Phase 1/Phase | |
| | 2013 WO2013138244 HERBST, | 2 A Clinical Study Using MEDI-551 in | |
| | Ronald, K . . . TREATMENT OF | Adult Subjects With Relapsed or | |
| | MULTIPLE SCLEROSIS WITH | Refractory Advanced B-Cell | |
| | ANTI-CD19 ANTIBODY | Malignancies | |
| | 2013 US20150044168 Herbst, | 2022 Bennett J L, Aktas . . . | |
| | Ronald; K . . . Treatment of | Association between B-cell | |
| | Multiple Sclerosis With Anti- | depletion and attack risk in | |
| | CD19 Antibody | neuromyelitis optica spectrum | |
| | 2012 US20130115657 | disorder: An exploratory analysis | |
| | Damschroder, Meli . . . | from N-MOmentum, a double- | |
| | HUMANIZED ANTI-CD19 | blind, randomised, placebo- | |
| | ANTIBODIES AND THEIR USE IN | controlled, multicentre phase 2/3 | |
| | TREATMENT OF ONCOLOGY, | trial. | |
| | TRANSPLANTATION AND | 2022 Nie T, Blair H A Inebilizumab: A | |
| | AUTOIMMUNE DISEASE | Review in Neuromyelitis Optica | |
| | 2012 U.S. Pat. No. 8,883,992 Damschroder, | Spectrum Disorder. | |
| | Meli . . . Humanized anti-CD19 | 2022 Yan L, Wang B, Sh . . . | |
| | antibodies | Pharmacodynamic modeling and | |
| | 2011 WO2012067981 HERBST, | exposure-response assessment of | |
| | Ronald, W . . . COMBINATION | inebilizumab in subjects with | |
| | THERAPY FOR B CELL | neuromyelitis optica spectrum | |
| | LYMPHOMAS | disorders. | |
| | 2011 US20130330328 Herbst, | 2021 Wingerchuk D M, Zh . . . | |
| | Ronald; W . . . Combination | Network Meta-analysis of Food and | |
| | Therapy For B Cell Lymphomas | Drug Administration-approved | |
| | 2010 WO2010102276 | Treatment Options for Adults with | |
| | SHARMA, Monika; S . . . | Aquaporin-4 Immunoglobulin G- | |
| | HUMANIZED ANTI-CD19 | positive Neuromyelitis Optica | |
| | ANTIBODY FORMULATIONS | Spectrum Disorder. | |
| | 2010 US20120148576 Sharma, | 2021 Yan L, Kimko H, W . . . | |
| | Monika S . . . HUMANIZED ANTI- | Population Pharmacokinetic | |
| | CD 19 ANTIBODY | Modeling of Inebilizumab in | |
| | FORMULATIONS | Subjects with Neuromyelitis Optica | |
| | 2007 WO2008031056 | Spectrum Disorders, Systemic | |
| | DAMSCHRODER, Meli . . . | Sclerosis, or Relapsing Multiple | |
| | HUMANIZED ANTI-CD19 | Sclerosis. | |
| | ANTIBODIES AND THEIR USE IN | 2021 Rensel M, Zabeti . . . Long-term | |
| | TREATMENT OF ONCOLOGY, | efficacy and safety of inebilizumab | |
| | TRANSPLANTATION AND | in neuromyelitis optica spectrum | |
| | AUTOIMMUNE DISEASE | disorder: Analysis of aquaporin-4- | |
| | 2007 U.S. Pat. No. 8,323,653 Damschroder, | immunoglobulin G-seropositive | |
| | Meli . . . Humanized anti-CD19 | participants taking inebilizumab for | |
| | antibodies and their use in | ≥4 years in the N-MOmentum trial. | |
| | treatment of oncology, | 2021 Ali F, Sharma K, . . . | |
| | transplantation and | Inebilizumab-cdon: USFDA approval | |
| | autoimmune disease | for the treatment of NMOSD | |
| | | (neuromyelitis optica spectrum | |
| | | disorder). | |
| | | 2021 Brod S A Immune | |
| | | reconstitution therapy in NMOSD. | |
| | | 2021 Marignier R, Benn . . . Disability | |
| | | Outcomes in the N-MOmentum | |
| | | Trial of Inebilizumab in | |
| | | Neuromyelitis Optica Spectrum | |
| | | Disorder. | |
| | | 2021 Cree B A, Bennett . . . Sensitivity | |
| | | analysis of the primary endpoint | |
| | | from the N-MOmentum study of | |
| | | inebilizumab in NMOSD. | |
| | | 2020 Valencia-Sanchez . . . Emerging | |
| | | Targeted Therapies for | |
| | | Neuromyelitis Optica Spectrum | |
| | | Disorders. | |
| | | 2020 Levy M, Fujihara . . . New | |
| | | therapies for neuromyelitis optica | |
| | | spectrum disorder. | |
| | | 2020 Frampton J E Inebilizumab: | |
| | | First Approval. | |
| | | 2020 Duchow A, Paul F, . . . Current | |
| | | and Emerging Biologics for the | |
| | | Treatment of Neuromyelitis Optica | |
| | | Spectrum Disorders. | |
| | | 2019 Ohmachi K, Ogura . . . A | |
| | | multicenter phase I study of | |
| | | inebilizumab, a humanized anti- | |
| | | CD19 monoclonal antibody, in | |
| | | Japanese patients with relapsed or | |
| | | refractory B-cell lymphoma and | |
| | | multiple myeloma. | |
| | | 2018 Streicher K, Srid . . . Baseline | |
| | | plasma cell gene signature predicts | |
| | | improvement in systemic sclerosis | |
| | | skin scores following treatment | |
| | | with inebilizumab (MEDI-551) and | |
| | | correlates with disease activity in | |
| | | systemic lupus and chronic | |
| | | obstructive pulmonary disease. | |
| | | 2016 Chen D, Gallagher . . . | |
| | | Inebilizumab, a B Cell-Depleting | |
| | | Anti-CD19 Antibody for the | |
| | | Treatment of Autoimmune | |
| | | Neurological Diseases: Insights from | |
| | | Preclinical Studies. | |
| | | 2016 Schiopu E, Chatte . . . Safety and | |
| | | tolerability of an anti-CD19 | |
| | | monoclonal antibody, MEDI-551, in | |
| | | subjects with systemic sclerosis: a | |
| | | phase I, randomized, placebo- | |
| | | controlled, escalating single-dose | |
| | | study. | |
| | | 2016 Gallagher S, Turm . . . | |
| | | Pharmacological profile of MEDI- | |
| | | 551, a novel anti-CD19 antibody, in | |
| | | human CD19 transgenic mice. | |
| | | 2014 Chen D, Blazek M, . . . Single | |
| | | Dose of Glycoengineered Anti-CD19 | |
| | | Antibody (MEDI551) Disrupts | |
| | | Experimental Autoimmune | |
| | | Encephalomyelitis by Inhibiting | |
| | | Pathogenic Adaptive Immune | |
| | | Responses in the Bone Marrow and | |
| | | Spinal Cord while Preserving | |
| | | Peripheral Regulatory Mechanisms. | |
| | | 2013 Matlawska-Wasowsk . . . | |
| | | Macrophage and NK-mediated | |
| | | killing of precursor-B acute | |
| | | lymphoblastic leukemia cells | |
| | | targeted with a-fucosylated anti- | |
| | | CD19 humanized antibodies. | |
| | | 2011 Ward E, Mitterede . . . A | |
| | | glycoengineered anti-CD19 | |
| | | antibody with potent antibody- | |
| | | dependent cellular cytotoxicity | |
| | | activity in vitro and lymphoma | |
| | | growth inhibition in vivo. | |
| | | 2010 Herbst R, Wang Y, . . . B-cell | |
| | | depletion in vitro and in vivo with | |
| | | an afucosylated anti-CD19 | |
| | | antibody. | |
| | | ASCO 2012 Phase I/II study of | |
| | | MEDI-551, a humanized | |
| | | monoclonal antibody targeting | |
| | | CD19, in subjects with relapsed or | |
| | | refractory advanced B-cell | |
| | | malignancies Trishna Goswami, . . . | |
| | | ASCO 2014 Phase 2 open-label | |
| | | study of MEDI-551 and | |
| | | bendamustine versus rituximab and | |
| | | bendamustine in adults with | |
| | | relapsed or refractory CLL. Douglas | |
| | | Edward GI . . . | |
| | | ASH 2013 Safety Profile and Clinical | |
| | | Response To MEDI-551, a | |
| | | Humanized Monoclonal Anti-CD19, | |
| | | In a Phase 1/2 Study In Adults With | |
| | | Relapsed Or Refractory Advanced B- | |
| | | Cell Malignancies Andres Forero- | |
| | | Tor . . . | |
| | | SITC 2013 Safety and disease | |
| | | response to MEDI-551, an anti- | |
| | | CD19 antibody, in chronic | |
| | | lymphocytic leukemia patients | |
| | | previously treated with rituximab | |
| | | Andres Forero, Me . . . | |
| Innovative | 2022 US20220249638 Wu, | | Innovative |
| Cell. Thera. patent | Zhao; Liu, Zh . . . HUMANIZED | | Cell. Thera. |
| anti-CD19 | ANTI-CD19 ANTIBODY AND USE | | |
| | THEREOF WITH CHIMERIC | | |
| | ANTIGEN RECEPTOR | | |
| | 2021 WO2021216731 PU, | | |
| | Chengfei, XIA . . . POLYSPECIFIC | | |
| | BINDING MOLECULES AND | | |
| | THEIR USE IN CELL THERAPY | | |
| | 2020 US20200385484 Xiao, Lei; | | |
| | Pu, Ch . . . USE OF CHIMERIC | | |
| | ANTIGEN RECEPTOR MODIFIED | | |
| | CELLS TO TREAT CANCER | | |
| | 2019 US20200030424 Wu, | | |
| | Zhao; Liu, Zh . . . HUMANIZED | | |
| | ANTI-CD19 ANTIBODY AND USE | | |
| | THEREOF WITH CHIMERIC | | |
| | ANTIGEN RECEPTOR | | |
| | 2019 U.S. Pat. No. 11,364,289 Wu, Zhao | | |
| | (Shangha . . . Humanized anti- | | |
| | CD19 antibody and use thereof | | |
| | with chimeric antigen receptor | | |
| | 2018 US20180153977 Wu, | | |
| | Zhao; Liu, Zh . . . HUMANIZED | | |
| | ANTI-CD19 ANTIBODY AND USE | | |
| | THEREOF WITH CHIMERIC | | |
| | ANTIGEN RECEPTOR | | |
| | 2018 U.S. Pat. No. 10,493,139 Wu, Zhao | | |
| | (Shangha . . . Humanized anti- | | |
| | CD19 antibody and use thereof | | |
| | with chimeric antigen receptor | | |
| JCAR014 | | 2013 NCT01865617 Phase 1/Phase | Juno |
| | | 2 Laboratory Treated T Cells in | |
| | | Treating Patients With Relapsed or | |
| | | Refractory Chronic Lymphocytic | |
| | | Leukemia, Non-Hodgkin | |
| | | Lymphoma, or Acute Lymphoblastic | |
| | | Leukemia | |
| JCAR015 | 2019 US20200172630 CHEN, | 2015 NCT02535364 Phase 2 Study | Juno Sloan- |
| | Yan; SHAMAH . . . ANTIBODIES | Evaluating the Efficacy and Safety of | Kettering |
| | AND CHIMERIC ANTIGEN | JCAR015 in Adult B-cell Acute | |
| | RECEPTORS SPECIFIC FOR CD19 | Lymphoblastic Leukemia (B-ALL) | |
| | 2019 WO2019213184 | 2013 NCT01840566 Phase 1 High | |
| | FRANKEL, Stanley . . . | Dose Therapy and Autologous Stem | |
| | COMBINATION THERAPY OF A | Cell Transplantation Followed by | |
| | CHIMERIC ANTIGEN RECEPTOR | Infusion of Chimeric Antigen | |
| | (CAR) T CELL THERAPY AND A | Receptor (CAR) Modified T-Cells | |
| | KINASE INHIBITOR | Directed Against CD19+ B-Cells for | |
| | 2016 WO2017096329 | Relapsed and Refractory Aggressive | |
| | THOMPSON, Lucas, . . . | B Cell Non-Hodgkin Lymphoma | |
| | MODIFIED CHIMERIC | 2010 NCT01087294 Phase 1 | |
| | RECEPTORS AND RELATED | Administration of Anti-CD19- | |
| | COMPOSITIONS AND | chimeric-antigen-receptor- | |
| | METHODS | transduced T Cells From the | |
| | 2016 US20190071487 Boice, | Original Transplant Donor to | |
| | Michael He . . . TNFRSF14/ | Patients With Recurrent or | |
| | HVEM PROTEINS AND | Persistent B-cell Malignancies After | |
| | METHODS OF USE THEREOF | Allogeneic Stem Cell | |
| | 2016 US20200017571 Boice, | Transplantation | |
| | Michael He . . . TNFRSF14/ | 2010 NCT01044069 Phase 1 | |
| | HVEM PROTEINS AND | Precursor B Cell Acute | |
| | METHODS OF USE THEREOF | Lymphoblastic Leukemia (B-ALL) | |
| | 2016 U.S. Pat. No. 11,261,232 Boice, | Treated With Autologous T Cells | |
| | Michael He . . . TNFRSF14/ | Genetically Targeted to the B Cell | |
| | HVEM proteins and methods of | Specific Antigen CD19 | |
| | use thereof | 2014 Davila M L, Rivier . . . Efficacy | |
| | 2015 WO2016033570 CHEN, | and toxicity management of 19-28z | |
| | Yan, SHAMA . . . ANTIBODIES | CAR T cell therapy in B cell acute | |
| | AND CHIMERIC ANTIGEN | lymphoblastic leukemia. | |
| | RECEPTORS SPECIFIC FOR CD19 | 2013 Davila M L, Kloss . . . CD19 CAR- | |
| | 2015 US20160152723 CHEN, | targeted T cells induce long-term | |
| | Yan; SHAMAH . . . ANTIBODIES | remission and B Cell Aplasia in an | |
| | AND CHIMERIC ANTIGEN | immunocompetent mouse model of | |
| | RECEPTORS SPECIFIC FOR CD19 | B cell acute lymphoblastic leukemia. | |
| | 2015 U.S. Pat. No. 10,533,055 Chen, Yan | View on Papers Page | |
| | (Lexing . . . Antibodies and | | |
| | chimeric antigen receptors | | |
| | specific for CD19 | | |
| | 2003 U.S. Pat. No. 7,446,190 Sadelain, | | |
| | Michel; . . . Nucleic acids | | |
| | encoding chimeric T cell | | |
| | receptors | | |
| | 2003 US20040043401 Sadelain; | | |
| | Michel; . . . Chimeric T cell | | |
| | receotors | | |
| Jiangsu Simcere | 2022 WO2023280297 XIN, Lian | | Jiangsu Simcere |
| patent anti-CD19 | CD19 ANTIBODY AND | | |
| | APPLICATION THEREOF | | |
| | 2021 WO2022105811 GE, Hu, | | |
| | XIN, Lian HUMANIZED CD19 | | |
| | ANTIBODY AND USE THEREOF | | |
| Jinan Taihe | 2019 WO2019214332 LI, Yina, | | Jinan Taihe |
| Pharma patent | WEI, Si . . . CD19-TARGETED | | Pharma |
| anti-CD19 | HUMAN ANTIBODY AND | | |
| | PREPARATION AND USE | | |
| | THEREOF | | |
| KTE-C19 | 2022 US20220403050 Sievers, | 2021 NCT04880434 Phase 2 Study | Kite |
| | Stuart; . . . ANTIGEN BINDING | to Evaluate the Efficacy of | |
| | MOLECULES AND METHODS OF | Brexucabtagene Autoleucel (KTE- | |
| | USE THEREOF | X19) in Participants With | |
| | 2020 US20210061910 | Relapsed/Refractory Mantle Cell | |
| | WILTZIUS, Jed J. W . . . | Lymphoma (Cohort 3) | |
| | Humanized Antigen-Binding | 2016 NCT02926833 Phase 1/Phase | |
| | Domains and Methods of Use | 2 A Study Evaluating KTE-C19 in | |
| | 2020 US20200354475 Wiltzius, | Combination With Atezolizumab in | |
| | Jed; Si . . . ANTIGEN BINDING | Subjects With Refractory Diffuse | |
| | MOLECULES SPECIFIC FOR AN | Large B-Cell Lymphoma (DLBCL) | |
| | ANTI-CD19 SCFV | 2015 NCT02625480 Phase 1/Phase | |
| | 2020 U.S. Pat. No. 11,384,155 Wiltzius, Jed | 2 A Multi-Center Study Evaluating | |
| | (Wi . . . Antigen binding | KTE-C19 in Pediatric and Adolescent | |
| | molecules specific for an anti- | Subjects With Relapsed/Refractory | |
| | CD19 scFv | B-precursor Acute Lymphoblastic | |
| | 2019 US20200246382 Perez, | Leukemia | |
| | Arianne; S . . . CHIMERIC | 2015 NCT02614066 Phase 1/Phase | |
| | ANTIGEN AND T CELL | 2 A Study Evaluating KTE-C19 in | |
| | RECEPTORS AND METHODS OF | Adult Subjects With | |
| | USE | Relapsed/Refractory B-precursor | |
| | 2018 US20180312588 Wiltzius, | Acute Lymphoblastic Leukemia (r/r | |
| | Jed J. W . . . HUMANIZED | ALL) (ZUMA-3) | |
| | ANTIGEN-BINDING DOMAINS | 2015 NCT02601313 Phase 2 A | |
| | AND METHODS OF USE | Phase 2 Multicenter Study | |
| | 2018 WO2018200496 | Evaluating Subjects With | |
| | WILTZIUS, Jed, J . . . HUMANIZED | Relapsed/Refractory Mantle Cell | |
| | ANTIGEN-BINDING DOMAINS | Lymphoma | |
| | AGAINST CD19 AND METHODS | 2015 NCT02348216 Phase 1/Phase | |
| | OF USE | 2 A Phase 1-2 Multi-Center Study | |
| | 2018 U.S. Pat. No. 10,844,120 Wiltzius, Jed | Evaluating KTE-C19 in Subjects With | |
| | J. . . . Humanized antigen- | Refractory Aggressive Non-Hodgkin | |
| | binding domains and methods | Lymphoma (ZUMA-1) | |
| | of use | 2022 Cohen J A, Ghobadi A | |
| | 2017 US20180086846 | Axicabtagene ciloleucel for the | |
| | WILTZIUS, Jed; SI . . . ANTIGEN | treatment of relapsed or refractory | |
| | BINDING MOLECULES AND | follicular lymphoma. | |
| | METHODS OF USE THEREOF | 2022 Shah B D, Smith N J . . . Cost- | |
| | 2017 U.S. Pat. No. 10,626,187 Wiltzius, Jed | Effectiveness of KTE-X19 for Adults | |
| | (Wo . . . Antigen binding | with Relapsed/Refractory B-Cell | |
| | molecules specific for an anti- | Acute Lymphoblastic Leukemia in | |
| | CD19 scFv | the United States. | |
| | | ASCO 2016 Ongoing complete | |
| | | remissions (CR) in the phase 1 of | |
| | | ZUMA-1: a phase 1-2 multicenter | |
| | | study evaluating the safety and | |
| | | efficacy of KTE-C19 (anti-CD19 CAR | |
| | | T cells) in subjects with refractory | |
| | | aggressive B-cell Non-Hodgkin | |
| | | Lymphoma (NHL Sattva Swarup | |
| | | Nee . . . | |
| | | ASCO 2017 Zuma-6: Phase 1-2 | |
| | | multicenter study evaluating safety | |
| | | and efficacy of axicabtagene | |
| | | ciloleucel (axi-cel; KTE-C19) in | |
| | | combination with atezolizumab in | |
| | | patients with refractory diffuse | |
| | | large b-cell lymphoma (DLBCL) | |
| | | Frederick Lundry . . . | |
| Li Huashun patent | 2019 WO2019137518 LI, | | |
| anti-CD19 | Huashun, REN, . . . SPECIFIC | | |
| | ANTIBODY TARGETING CD19 | | |
| | AND PREPARATION METHOD | | |
| | THEREFOR AS WELL AS | | |
| | APPLICATION THEREOF, AND | | |
| | CAR-NK CELL TARGETING CD19 | | |
| | AND PREPARATION METHOD | | |
| | THEREFOR AS WELL AS | | |
| | APPLICATION THEREOF | | |
| Lilly patent anti- | 2021 US20210269520 Allan, | | Lilly |
| CD19 | Barrett; B . . . ANTI-HUMAN CD19 | | |
| | ANTIBODIES | | |
| | 2021 WO2021173471 ALLAN, | | |
| | Barrett, B . . . ANTI-HUMAN CD19 | | |
| | ANTIBODIES | | |
| Lisocabtagene | | 2020 NCT04245839 Phase 2 A Study | BMS Juno |
| Maraleucel | | to Evaluate the Efficacy and Safety | |
| | | of JCAR017 in Adult Subjects With | |
| | | Relapsed or Refractory Indolent B- | |
| | | cell Non-Hodgkin Lymphoma (NHL) | |
| | | 2018 NCT03744676 Phase 2 A | |
| | | Safety Trial of Lisocabtagene | |
| | | Maraleucel (JCAR017) for Relapsed | |
| | | and Refractory (R/R) B-cell Non- | |
| | | Hodgkin Lymphoma (NHL) in the | |
| | | Outpatient Setting (TRANSCEND- | |
| | | OUTREACH-007) | |
| | | 2018 NCT03575351 Phase 3 A Study | |
| | | to Compare the Efficacy and Safety | |
| | | of JCAR017 to Standard of Care in | |
| | | Adult Subjects With High-risk, | |
| | | Transplant-eligible Relapsed or | |
| | | Refractory Aggressive B-cell Non- | |
| | | Hodgkin Lymphomas | |
| | | 2018 NCT03743246 Phase 1/Phase | |
| | | 2 A Study to Evaluate the Safety and | |
| | | Efficacy of JCAR017 in Pediatric | |
| | | Subjects With Relapsed/Refractory | |
| | | (r/r) B-cell Acute Lymphoblastic | |
| | | Leukemia (B-ALL) and B-cell Non- | |
| | | Hodgkin Lymphoma (B-NHL) | |
| | | 2018 NCT03483103 Phase 2 | |
| | | Lisocabtagene Maraleucel | |
| | | (JCAR017) as Second-Line Therapy | |
| | | (TRANSCEND-PILOT-017006) | |
| | | 2018 NCT03484702 Phase 2 Trial to | |
| | | Determine the Efficacy and Safety | |
| | | of JCAR017 in Adult Subjects With | |
| | | Aggressive B-Cell Non-Hodgkin | |
| | | Lymphoma | |
| | | 2018 NCT03436771 Long-term | |
| | | Follow-up Study for Patients | |
| | | Previously Treated With a Juno CAR | |
| | | T-Cell Product | |
| | | 2017 NCT03310619 Phase 1/Phase | |
| | | 2 A Safety and Efficacy Trial of | |
| | | JCAR017 Combinations in Subjects | |
| | | With Relapsed/Refractory B-cell | |
| | | Malignancies (PLATFORM) | |
| | | 2015 NCT02631044 Phase 1 Study | |
| | | Evaluating the Safety and | |
| | | Pharmacokinetics of JCAR017 in B- | |
| | | cell Non-Hodgkin Lymphoma (NHL) | |
| | | 2014 NCT02028455 Phase 1/Phase | |
| | | 2 A Pediatric and Young Adult Trial | |
| | | of Genetically Modified T Cells | |
| | | Directed Against CD19 for | |
| | | Relapsed/Refractory CD19+ | |
| | | Leukemia | |
| | | 2012 NCT01683279 Phase 1 A | |
| | | Pediatric Trial of Genetically | |
| | | Modified Autologous T Cells | |
| | | Directed Against CD19 for Relapsed | |
| | | CD19+ Acute Lymphoblastic | |
| | | Leukemia | |
| | | NCT04400591 Nonconforming | |
| | | Lisocabtagene Maraleucel | |
| | | Expanded Access Protocol | |
| | | ASCO 2017 CR rates in | |
| | | relapsed/refractory (R/R) aggressive | |
| | | B-NHL treated with the CD19- | |
| | | directed CAR T-cell product | |
| | | JCAR017 (TRANSCEND NHL 001) | |
| | | Jeremy S. Abramso . . . | |
| | | ASH 2016 Preclinical Analyses | |
| | | Support Clinical Investigation of | |
| | | Combined Anti-CD19 CAR-T Cell, | |
| | | JCAR017 with Ibrutinib for the | |
| | | Treatment of Chronic Lymphocytic | |
| | | Leukemia Jim Qin, Alex Bat . . . | |
| Ioncastuximab | 2022 WO2023274974 VAN | 2023 NCT05672251 Phase 2 | ADC Therapeutics |
| tesirine-lpyl | BERKEL, Patri . . . COMBINATION | Loncastuximab Tesirine and | Medimmune |
| | THERAPY USING ANTIBODY- | Mosunetuzumab for the Treatment | |
| | DRUG CONJUGATES | of Relapsed or Refractory Diffuse | |
| | 2022 US20220280651 Feingold, | Large B-Cell Lymphoma | |
| | Jay Mar . . . DOSAGE REGIMES | 2022 NCT05144009 Phase 2 A Study | |
| | FOR THE ADMINISTRATION OF | of Loncastuximab Tesirine and | |
| | AN ANTI-CD19 ADC | Rituximab (Lonca-R) in Previously | |
| | 2020 US20200345861 VAN | Untreated Unfit/Frail Participants | |
| | BERKEL, PATRI . . . | With Diffuse Large B-cell Lymphoma | |
| | PYRROLOBENZODIAZEPINE- | (DLBCL) | |
| | ANTIBODY CONJUGATES | 2021 NCT04699461 Phase 2 Study | |
| | 2020 WO2020249528 | to Evaluate the Efficacy and Safety | |
| | ZAMMARCHI, France . . . | of Loncastuximab Tesirine Versus | |
| | COMBINATION THERAPY | Idelalisib in Participants With | |
| | COMPRISING AN ANTI-CD19 | Relapsed or Refractory Follicular | |
| | ANTIBODY DRUG CONJUGATE | Lymphoma | |
| | AND A PI3K INHIBITOR OR A | 2020 NCT04384484 Phase 3 Study | |
| | SECONDARY AGENT | to Evaluate Loncastuximab Tesirine | |
| | 2020 US20220305132 | With Rituximab Versus | |
| | ZAMMARCHI, France . . . | Immunochemotherapy in | |
| | COMBINATION THERAPY | Participants With Relapsed or | |
| | COMPRISING AN ANTI-CD19 | Refractory Diffuse Large B-Cell | |
| | ANTIBODY DRUG CONJUGATE | Lymphoma | |
| | AND A PI3K INHIBITOR OR A | 2018 NCT03685344 Phase 1 Safety | |
| | SECONDARY AGENT | and Antitumor Activity Study of | |
| | 2019 WO2020109251 | Loncastuximab Tesirine and | |
| | FEINGOLD, Jay Mar . . . DOSAGE | Durvalumab in Diffuse Large B-Cell, | |
| | REGIME | Mantle Cell, or Follicular Lymphoma | |
| | 2019 WO2020043878 | 2018 NCT03589469 Phase 2 Study | |
| | ZAMMARCHI, France . . . | to Evaluate the Efficacy and Safety | |
| | COMBINATION THERAPY | of Loncastuximab Tesirine in | |
| | 2019 US20210322564 | Patients With Relapsed or | |
| | ZAMMARCHI, France . . . | Refractory Diffuse Large B-Cell | |
| | COMBINATION THERAPY | Lymphoma | |
| | 2018 WO2018229222 | 2016 NCT02669017 Phase 1 Study | |
| | FEINGOLD, Jay Mar . . . DOSAGE | of ADCT-402 in Patients With | |
| | REGIMES FOR THE | Relapsed or Refractory B-cell | |
| | ADMINISTRATION OF AN ANTI- | Lineage Non Hodgkin Lymphoma | |
| | CD19 ADC | (B-NHL) | |
| | 2018 US20200171164 Feingold, | 2016 NCT02669264 Phase 1 Study | |
| | Jay Mar . . . DOSAGE REGIMES | of ADCT-402 in Patients With | |
| | FOR THE ADMINISTRATION OF | Relapsed or Refractory B-cell | |
| | AN ANTI-CD19 ADC | Lineage Acute Lymphoblastic | |
| | 2018 U.S. Pat. No. 11,318,211 Feingold, Jay | Leukemia (B-ALL) | |
| | Mar . . . Dosage regimes for the | 2023 Tiberghien A C, Vi . . . | |
| | administration of an anti-CD19 | Comparison of | |
| | ADC | Pyrrolobenzodiazepine Dimer Bis- | |
| | 2018 WO2018193105 | imine versus Mono-imine: DNA | |
| | FEINGOLD, Jay Mar . . . | Interstrand Cross-linking, | |
| | COMBINATION THERAPY | Cytotoxicity, Antibody-Drug | |
| | 2018 US20200405879 Feingold, | Conjugate Efficacy and Toxicity. | |
| | Jay Mar . . . COMBINATION | 2022 Westin J, Burke J . . . ABCL-272 | |
| | THERAPY | A Phase 2, Open-Label Study of | |
| | 2018 US20180250417 VAN | Loncastuximab Tesirine in | |
| | BERKEL, PATRI . . . | Combination With Rituximab | |
| | PYRROLOBENZODIAZEPINE- | (Lonca-R) in Previously Untreated | |
| | ANTIBODY CONJUGATES | Unfit/Frail Patients With Diffuse | |
| | 2018 U.S. Pat. No. 10,780,181 Van Berkel, | Large B-Cell Lymphoma (LOTIS-9). | |
| | Patri . . . Pyrrolobenzodiazepine- | 2022 Spira A, Zhou X, . . . ABCL-310 | |
| | antibody conjugates | Health-Related Quality of Life and | |
| | 2016 WO2016166298 VAN | Tolerability in Patients | |
| | BERKEL, Patri . . . SITE-SPECIFIC | With/Without Skin Toxicity During | |
| | ANTIBODY-DRUG CONJUGATES | Loncastuximab Tesirine Treatment | |
| | 2014 WO2015052534 | in a Phase 2 Clinical Trial (LOTIS-2). | |
| | HOWARD, Philip Wi . . . | 2022 Kingsley E, Grosi . . . ABCL-320 | |
| | PYRROLOBENZODIAZEPINE- | Initial Safety Run-In Results of the | |
| | ANTIBODY CONJUGATES | Phase 3 LOTIS-5 Trial: Novel | |
| | 2014 US20160263242 Howard, | Combination of Loncastuximab | |
| | Philip Wi . . . | Tesirine With Rituximab (Lonca-R) | |
| | PYRROLOBENZODIAZEPINE- | Versus Immunochemotherapy in | |
| | ANTIBODY CONJUGATES | Patients With R/R DLBCL. | |
| | 2014 U.S. Pat. No. 9,950,078 Howard, | 2022 Hamadani M, Graha . . . ABCL- | |
| | Philip Wi . . . | 334 Long-Term Survival Projections | |
| | Pyrrolobenzodiazepine- | of Loncastuximab Tesirine-Treated | |
| | antibody conjugates | Patients in Relapsed or Refractory | |
| | 2013 WO2014057117 VAN | Diffuse Large B-Cell Lymphoma. | |
| | BERKEL, Patri . . . | 2022 Alderuccio J P, Ai . . . | |
| | PYRROLOBENZODIAZEPINE- | Loncastuximab tesirine in | |
| | ANTIBODY CONJUGATES | relapsed/refractory high-grade B- | |
| | 2013 US20150283262 Van | cell lymphoma: a subgroup analysis | |
| | Berkel, Patri . . . | from the LOTIS-2 study. | |
| | PYRROLOBENZODIAZEPINE- | 2022 Calabretta E, Ham . . . THE | |
| | ANTIBODY CONJUGATES | ANTIBODY-DRUG CONJUGATE | |
| | 2013 U.S. Pat. No. 9,931,414 Van Berkel, | LONCASTUXIMAB TESIRINE FOR | |
| | Patri . . . Pyrrolobenzodiazepine- | THE TREATMENT OF DIFFUSE LARGE | |
| | antibody conjugates | B-CELL LYMPHOMA. | |
| | | 2022 Hamadani M, Chen . . . | |
| | | Matching-adjusted Indirect | |
| | | Comparison of the Efficacy of | |
| | | Loncastuximab Tesirine Versus | |
| | | Treatment in the | |
| | | Chemoimmunotherapy Era for | |
| | | Relapsed/Refractory Diffuse Large | |
| | | B-cell Lymphoma. | |
| | | 2022 Furqan F, Hamadani M | |
| | | Loncastuximab tesirine in relapsed | |
| | | or refractory diffuse large B-cell | |
| | | lymphoma: a review of clinical data. | |
| | | 2022 Xu B Loncastuximab tesirine: | |
| | | an effective therapy for relapsed or | |
| | | refractory diffuse large B-cell | |
| | | lymphoma. | |
| | | 2022 Gettman L New Drug Update: | |
| | | Dostarlimab, Loncastuximab | |
| | | Tesirine, and Aducanumab. | |
| | | 2021 Hess B, Townsend . . . Efficacy | |
| | | and Safety Exposure-Response | |
| | | Analysis of Loncastuximab Tesirine | |
| | | in Patients with B cell non-Hodgkin | |
| | | Lymphoma. | |
| | | 2021 Caimi P F, Ardeshn . . . The | |
| | | AntiCD19 Antibody Drug | |
| | | Immunoconjugate Loncastuximab | |
| | | Achieves Responses in DLBCL | |
| | | Relapsing After AntiCD19 CAR-T Cell | |
| | | Therapy. | |
| | | 2021 Ahmed N, Hamadani M | |
| | | Evaluating efficacy and safety of | |
| | | loncastuximab tesirine injection for | |
| | | the treatment of adult patients with | |
| | | relapsed or refractory large B-cell | |
| | | lymphoma. | |
| | | 2021 Goparaju K, Caimi P F | |
| | | Loncastuximab tesirine for | |
| | | treatment of relapsed or refractory | |
| | | diffuse large B cell lymphoma. | |
| | | 2021 Lee A Loncastuximab Tesirine: | |
| | | First Approval. | |
| | | 2021 Caimi P F, Ai W, A . . . | |
| | | Loncastuximab tesirine in relapsed | |
| | | or refractory diffuse large B-cell | |
| | | lymphoma (LOTIS-2): a multicentre, | |
| | | open-label, single-arm, phase 2 | |
| | | trial. | |
| | | 2020 Hamadani M, Radfo . . . Final | |
| | | Results of a Phase 1 Study of | |
| | | Loncastuximab Tesirine in | |
| | | Relapsed/Refractory B-Cell Non- | |
| | | Hodgkin Lymphoma. | |
| | | 2020 Corbett S, Huang . . . The role | |
| | | of specific ATP-binding cassette | |
| | | transporters in the acquired | |
| | | resistance to | |
| | | pyrrolobenzodiazepine dimer- | |
| | | containing antibody-drug | |
| | | conjugates. | |
| | | 2020 Jain N, Stock W, . . . | |
| | | Loncastuximab tesirine, an anti- | |
| | | CD19 antibody-drug conjugate, in | |
| | | relapsed/refractory B-cell acute | |
| | | lymphoblastic leukemia. | |
| | | 2018 Zammarchi F, Corb . . . ADCT- | |
| | | 402, a PBD dimer-containing | |
| | | antibody drug conjugate targeting | |
| | | CD19-expressing malignancies. | |
| | | AACR 2017 Characterization of the | |
| | | mechanism of action, | |
| | | pharmacodynamics and preclinical | |
| | | safety of ADCT-402, a | |
| | | pyrrolobenzodiazepine (PBD) | |
| | | dimer-containing antibody-drug | |
| | | conjugate (ADC) targeting CD19- | |
| | | expressing hematological | |
| | | malignancies Francesca Zammarc . . . | |
| | | ASCO 2016 A phase 1 adaptive | |
| | | dose-escalation study to evaluate | |
| | | the tolerability, safety, | |
| | | pharmacokinetics, and antitumor | |
| | | activity of ADCT-402 in patients | |
| | | with relapsed or refractory B-cell | |
| | | lineage non Hodgkin lymphoma (B- | |
| | | NHL) Ki Y. Chung, Mehd . . . | |
| | | ASH 2015 Pre-Clinical Development | |
| | | of Adct-402, a Novel | |
| | | Pyrrolobenzodiazepine (PBD)-Based | |
| | | Antibody Drug Conjugate (ADC) | |
| | | Targeting CD19-Expressing B-Cell | |
| | | Malignancies Francesca Zammarc . . . | |
| | | ASH 2017 Encouraging Early Results | |
| | | from the First in-Human Clinical | |
| | | Trial of Adct-402 (Loncastuximab | |
| | | Tesirine), a Novel | |
| | | Pyrrolobenzodiazepine-Based | |
| | | Antibody Drug Conjugate, in | |
| | | Relapsed/Refractory B-Cell Lineage | |
| | | Non-Hodgkin Lymphoma Brad S. | |
| | | Kahl, MD, . . . | |
| | | ASH 2017 Interim Data from a | |
| | | Phase 1 Study Evaluating | |
| | | Pyrrolobenzodiazepine-Based | |
| | | Antibody Drug Conjugate Adct-402 | |
| | | (Loncastuximab Tesirine) Targeting | |
| | | CD19 for Relapsed or Refractory B- | |
| | | Cell Acute Lymphoblastic Leukemia | |
| | | Nitin Jain, MD, R . . . | |
| MDX-1342 | 2007 WO2009054863 KING, | 2008 NCT00593944 Phase 1 Study | Medarex |
| | David, John . . . HUMAN | of MDX-1342 in Patients With | |
| | ANTIBODIES THAT BIND CD19 | Chronic Lymphocytic Leukemia | |
| | AND USES THEREOF | (CLL) | |
| | 2007 US20100104509 King, | 2008 NCT00639834 Phase 1 Study | |
| | David John; . . . HUMAN | of the Safety of MDX-1342 in | |
| | ANTIBODIES THAT BIND CD19 | Combination With Methotrexate in | |
| | AND USES THEREOF | Patients With Rheumatoid Arthritis | |
| | 2006 WO2007002223 RAO- | 2009 Cardarelli P M, Ra . . . A | |
| | NAIK, Chetana . . . CD19 | nonfucosylated human antibody to | |
| | ANTIBODIES AND THEIR USES | CD19 with potent B-cell depletive | |
| | | activity for therapy of B-cell | |
| | | malignancies. | |
| Merck patent | 2015 US20150322159 Super, | 2013 NCT01805375 Phase 1 A | Merck Serono |
| anti-CD19 | Michael; D . . . METHODS OF USE | Phase I Trial of DI-B4 in Patients | |
| | OF ANTI-CD19 ANTIBODIES | With Advanced CD19 Positive | |
| | WITH REDUCED | Indolent B-cell Malignancies | |
| | IMMUNOGENICITY | | |
| | 2015 U.S. Pat. No. 10,072,092 Super, | | |
| | Michael (L . . . Methods of use of | | |
| | anti-CD19 antibodies with | | |
| | reduced immunogenicity | | |
| | 2014 US20140288288 Super, | | |
| | Michael; D . . . ANTI-CD19 | | |
| | ANTIBODIES WITH REDUCED | | |
| | IMMUNOGENICITY | | |
| | 2014 U.S. Pat. No. 8,957,195 Super, | | |
| | Michael; D . . . Anti-CD19 | | |
| | antibodies with reduced | | |
| | immunogenicity | | |
| | 2006 US20070154473 Super, | | |
| | Michael; D . . . Anti-CD19 | | |
| | antibodies with reduced | | |
| | immunogenicity | | |
| | 2006 U.S. Pat. No. 8,691,952 Super, | | |
| | Michael; D . . . Anti-CD19 | | |
| | antibodies with reduced | | |
| | immunogenicity | | |
| | 2006 WO2007076950 SUPER, | | |
| | Michael; D . . . ANTI-CD19 | | |
| | ANTIBODIES WITH REDUCED | | |
| | IMMUNOGENICITY | | |
| Millenium patent | 2021 WO2021217024 | | Millennium |
| anti-CD19 | TAVARES, Daniel, . . . ANTI-CD19 | | |
| | ANTIBODIES AND USES | | |
| | THEREOF | | |
| | 2021 US20210347888 Tavares, | | |
| | Daniel; . . . ANTI-CD19 | | |
| | ANTIBODIES AND USES THEROF | | |
| Nanjing Bioheng | 2021 WO2022121880 ZHOU, | | Nanjing Bioheng |
| Bio patent anti- | Yali, JIANG . . . CD19-TARGETING | | Bio |
| CD19 | HUMANIZED ANTIBODY AND | | |
| | USE THEREOF | | |
| Nanjing Legend | 2021 WO2022012683 FAN, | | Nanjing Legend |
| Bio patent anti- | Xiaohu, ZHOU . . . CD19 BINDING | | Bio |
| CD19 CAR | MOLECULES AND USES | | |
| | THEREOF | | |
| | 2021 WO2022012681 FAN, | | |
| | Xiaohu, ZHOU . . . MULTISPECIFIC | | |
| | CHIMERIC ANTIGEN | | |
| | RECEPTORS AND USES | | |
| | THEREOF | | |
| | 2017 US20190321404 FAN, | | |
| | Xiaohu; ZHUA . . . NOVEL | | |
| | CHIMERIC ANTIGEN RECEPTOR | | |
| | AND USE THEREOF | | |
| NCI anti-CD19 | 2021 WO2021161197 KUMAR, | 2021 NCT05052528 Phase 1 | NCI |
| CAR | Lalit ANTI-IDIOTYPE | Fludarabine and Cyclophosphamide | |
| | ANTIBODIES TARGETING ANTI- | With or Without Rituximab Before | |
| | CD19 CHIMERIC ANTIGEN | CD19 Chimeric Antigen Receptor T | |
| | RECEPTOR | Cells for the Treatment of Relapsed | |
| | | or Refractory Diffuse Large B-Cell | |
| | | Lymphoma | |
| | | 2020 NCT04544592 Phase 1/Phase | |
| | | 2 UCD19 CarT in Treatment of | |
| | | Pediatric B-ALL and B-NHL | |
| | | 2012 NCT01593696 Phase 1 Anti- | |
| | | CD19 White Blood Cells for Children | |
| | | and Young Adults With B Cell | |
| | | Leukemia or Lymphoma | |
| | | 2009 NCT00924326 Phase 1 CAR T | |
| | | Cell Receptor Immunotherapy for | |
| | | Patients With B-cell Lymphoma | |
| | | 2016 Brudno J N, Somerv . . . | |
| | | Allogeneic T Cells That Express an | |
| | | Anti-CD19 Chimeric Antigen | |
| | | Receptor Induce Remissions of B- | |
| | | Cell Malignancies That Progress | |
| | | After Allogeneic Hematopoietic | |
| | | Stem-Cell Transplantation Without | |
| | | Causing Graft-Versus-Host Disease. | |
| | | 2014 Lee D W, Kochender . . . T cells | |
| | | expressing CD19 chimeric antigen | |
| | | receptors for acute lymphoblastic | |
| | | leukaemia in children and young | |
| | | adults: a phase 1 dose-escalation | |
| | | trial. | |
| | | 2010 Kochenderfer J N, . . . | |
| | | Eradication of B-lineage cells and | |
| | | regression of lymphoma in a patient | |
| | | treated with autologous T cells | |
| | | genetically engineered to recognize | |
| | | CD19. | |
| NKX019 | 2020 WO2020180882 TRAGER, | | Nkarta |
| | James, Ba . . . CD19-DIRECTED | | |
| | CHIMERIC ANTIGEN | | |
| | RECEPTORS AND USES | | |
| | THEREOF IN IMMUNOTHERAPY | | |
| obexelimab | 2021 WO2022076573 DING, | 2022 NCT05662241 Phase 3 A | Amgen Xencor |
| | Ying, DESJA . . . BIOMARKERS, | Phase 3 Study of Obexelimab in | |
| | METHODS, AND | Patients With IgG4-Related Disease | |
| | COMPOSITIONS FOR TREATING | 2016 NCT02867098 Phase 1 | |
| | AUTOIMMUNE DISEASE | XmAb5871 Bioavailability Study | |
| | INCLUDING SYSTEMIC LUPUS | 2016 NCT02725476 Phase 2 Study | |
| | ERYTHEMATOUS (SLE) | to Evaluate the Effect of | |
| | 2017 US20180009900 Bernett, | XmAb ®5871 on Disease Activity in | |
| | Matthew . . . OPTIMIZED | Patients With IgG4-Related Disease | |
| | ANTIBODIES THAT TARGET | (RD) | |
| | CD19 | 2016 NCT02725515 Phase 2 A Study | |
| | 2017 US20180148511 Bernett, | of the Effect of XmAb ®5871 in | |
| | Matthew . . . OPTIMIZED | Patients With Systemic Lupus | |
| | ANTIBODIES THAT TARGET | Erythematosus | |
| | CD19 | 2014 Szili D, Cserhalm . . . | |
| | 2017 U.S. Pat. No. 10,626,182 Bernett, | Suppression of innate and adaptive | |
| | Matthew . . . Optimized | B cell activation pathways by | |
| | antibodies that target CD19 | antibody coengagement of FcγRIIb | |
| | 2011 U.S. Pat. No. 8,362,210 Lazar, | and CD19. | |
| | Gregory A . . . Antibody variants | 2011 Horton H M, Chu S Y . . . | |
| | with enhanced complement | Antibody-Mediated Coengagement | |
| | activity | of Fc{gamma}RIIb and B Cell | |
| | 2007 WO2008022152 | Receptor Complex Suppresses | |
| | BERNETT, Matthew . . . | Humoral Immunity in Systemic | |
| | OPTIMIZED ANTIBODIES THAT | Lupus Erythematosus. | |
| | TARGET CD19 | | |
| | 2007 US20080260731 Bernett, | | |
| | Matthew . . . OPTIMIZED | | |
| | ANTIBODIES THAT TARGET | | |
| | CD19 | | |
| | 2007 U.S. Pat. No. 8,524,867 Bernett, | | |
| | Matthew . . . Optimized | | |
| | antibodies that target CD19 | | |
| Oslo U. patent | 2019 WO2020011706 | | Oslo U. |
| anti-CD19 CAR | KVALHEIM, Gunnar, . . . TWO | | |
| | CHIMERIC ANTIGEN | | |
| | RECEPTORS SPECIFICALLY | | |
| | BINDING CD19 AND IGKAPPA | | |
| PCAR-019 | | 2016 NCT02851589 Phase 1/Phase | PersonGen |
| | | 2 Study Evaluating the Efficacy and | |
| | | Safety of PCAR-019 in CD19 Positive | |
| | | Relapsed or Refractory Leukemia | |
| | | and Lymphoma | |
| Persongen patent | 2016 WO2016112855 YANG, | | PersonGen |
| anti-CD19 | Lin, CHEN, . . . ANTI-CD19 | | |
| | MONOCLONAL ANTIBODY AND | | |
| | PREPARATION METHOD | | |
| | THEREFOR | | |
| Precision Biotech | 2018 WO2018201794 ZHANG, | | Precision Biotech |
| patent anti-CD19 | Wei, [symbol] CHIMERIC ANTIGEN | | |
| CAR | RECEPTOR AGAINST HUMAN | | |
| | CD19 ANTIGEN AND | | |
| | APPLICATION THEREOF | | |
| Promab Bio | 2021 WO2022026330 WU, | | ProMab Bio |
| patent anti-CD19 | Lijun, GOLUBO . . . HUMANIZED | | |
| CAR | CD37 AND BI-SPECIFIC CD19- | | |
| | HUMANIZED CD37 CAR-T CELLS | | |
| | 2020 US20200354451 Wu, | | |
| | Lijun; Golubo . . . CHIMERIC | | |
| | ANTIGEN RECEPTORS | | |
| | COMPRISING A HUMAN | | |
| | TRANSFERRIN EPITOPE | | |
| | SEQUENCE | | |
| | 2020 WO2020180551 WU, | | |
| | Lijun, GOLUBO . . . CAR-T CELLS | | |
| | WITH HUMANIZED CD19 SCFV | | |
| | 2020 WO2020163222 WU, | | |
| | Lijun, GOLUBO . . . NUCLEIC ACID | | |
| | SEQUENCE ENCODING | | |
| | CHIMERIC ANTIGEN RECEPTOR | | |
| | AND A SHORT HAIRPIN RNA | | |
| | SEQUENCE FOR IL-6 OR A | | |
| | CHECKPOINT INHIBITOR | | |
| PZ01 | | 2017 NCT03281551 Phase 1 Efficacy | Pinze Lifetech |
| | | and Safety of PZ01 Treatment in | |
| | | Patients With r/r CD19+ B-cell | |
| | | Acute Lymphoblastic Leukemia/B | |
| | | Cell Lymphoma | |
| RD126 | 2020 WO2020233589 TAN, | | Iaso |
| | Taochao, DAI . . . FULLY HUMAN | | |
| | ANTIBODY TARGETING CD19 | | |
| | AND APPLICATION THEREOF | | |
| | 2020 US20220220200 Tan, | | |
| | Taochao; Dai . . . FULLY HUMAN | | |
| | ANTIBODY TARGETING CD19 | | |
| | AND APPLICATION THEREOF | | |
| RemeGen RC58 | | 2018 Li Z, Wang M, Yao . . . | RemeGen |
| | | Development of novel anti-CD19 | |
| | | antibody-drug conjugates for B-cell | |
| | | lymphoma treatment. | |
| Roche Glycart | 2022 US20220204616 Ferrara | | Glycart |
| patent anti-CD19 | Koller, C . . . HUMANIZED ANTI- | | |
| | HUMAN CD19 ANTIBODIES | | |
| | AND METHODS OF USE | | |
| | 2019 US20200317774 Hofer, | | |
| | Thomas; Fe . . . ANTI-HUMAN | | |
| | CD19 ANTIBODIES WITH HIGH | | |
| | AFFINITY | | |
| | 2018 US20180230215 HOFER, | | |
| | Thomas; FE . . . ANTI-HUMAN | | |
| | CD19 ANTIBODIES WITH HIGH | | |
| | AFFINITY | | |
| | 2016 WO2017055541 | | |
| | GEORGES, Guy, MOE . . . | | |
| | HUMANIZED ANTI-HUMAN | | |
| | CD19 ANTIBODIES AND | | |
| | METHODS OF USE | | |
| | 2016 US20180282409 FERRARA | | |
| | KOLLER, C . . . HUMANIZED ANTI- | | |
| | HUMAN CD19 ANTIBODIES | | |
| | AND METHODS OF USE | | |
| | 2016 U.S. Pat. No. 11,286,300 Ferrara | | |
| | Koller, C . . . Humanized anti- | | |
| | human CD19 antibodies and | | |
| | methods of use | | |
| | 2016 WO2017055328 HOFER, | | |
| | Thomas, FE . . . ANTI-HUMAN | | |
| | CD19 ANTIBODIES WITH HIGH | | |
| | AFFINITY | | |
| | 2011 WO2011147834 | | |
| | DIMOUDIS, Nikolao . . . | | |
| | ANTIBODIES AGAINST CD19 | | |
| | AND USES THEREOF | | |
| Second | | 2015 NCT02644655 Phase 1/Phase | Second Military |
| Mil. Med. U. China | | 2 Immunotherapy Using Autologous | Med Univ |
| anti-CD19 CAR | | T Cell-Engineered With CD19- | |
| | | specific Chimeric Antigen Receptor | |
| | | for the Treatment of Recurrent/ | |
| | | Refractory B Cell Leukemia | |
| SGN-CD19B | | 2016 NCT02702141 Phase 1 A | Seattle Genetics |
| | | Safety Study of SGN-CD19B in | |
| | | Patients With B-cell Non-Hodgkin | |
| | | Lymphoma | |
| | | 2017 Ryan M C, Palanca- . . . | |
| | | Therapeutic potential of SGN- | |
| | | CD19B, a PBD-based anti-CD19 drug | |
| | | conjugate, for treatment of B-cell | |
| | | malignancies. | |
| | | ASH 2015 SGN-CD19B, a | |
| | | Pyrrolobenzodiazepine (PBD)-Based | |
| | | Anti-CD19 Drug Conjugate, | |
| | | Demonstrates Potent Preclinical | |
| | | Activity Against B-Cell Malignancies | |
| | | Maureen C. Ryan, . . . | |
| Shanghai | | 2017 NCT03030976 Phase 1 A Study | Shanghai |
| GeneChem anti- | | of CD19 Redirected Autologous T | GeneChem |
| CD19 CAR | | Cells for CD19 Positive Systemic | |
| | | Lupus Erythematosus (SLE) | |
| Shanghai SiDanSai | 2017 WO2018126369 WU, | | Shanghai |
| patent anti-CD19 | Zhao, LIU, Zh . . . HUMANIZED | | SiDanSai Bio. |
| | ANTI-CD19 ANTIBODY AND USE | | |
| | THEREOF WITH CHIMERIC | | |
| | ANTIGEN RECEPTOR | | |
| | 2015 WO2017015783 WU, | | |
| | Zhao, LIU, Zh . . . HUMANIZED | | |
| | ANTI-CD19 ANTIBODY AND USE | | |
| | THEREOF | | |
| Shanghai SiDanSai | 2016 WO2016138846 WU, | | Shanghai |
| patent anti-CD19 | Zhao REDUCING IMMUNE | | SiDanSai Bio. |
| CAR | TOLERANCE INDUCED BY PD-L1 | | |
| Sloan-Kettering | 2020 WO2020210232 CHEUNG, | | Sloan-Kettering |
| patent anti-CD19 | Nai-Kong . . . CD19 ANTIBODIES | | |
| | AND METHODS OF USING THE | | |
| | SAME | | |
| | 2020 US20220177579 | | |
| | CHEUNG, Nai-Kong . . . CD19 | | |
| | ANTIBODIES AND METHODS OF | | |
| | USING THE SAME | | |
| Sorrento patent | 2021 WO2022081486 MA, | | Sorrento |
| anti-CD19 CAR | Qiangzhong, G . . . CD19- | | |
| | DIRECTED CHIMERIC ANTIGEN | | |
| | RECEPTOR CONSTRUCTS | | |
| Southwest Hosp., | | 2016 NCT02737085 Phase 1/Phase | Southwest Hosp., |
| China anti-CD19 | | 2 the Sequential Therapy of CD19- | China |
| CAR | | targeted and CD20-targeted CAR-T | |
| | | Cell Therapy for Diffuse Large B Cell | |
| | | Lymphoma(DLBCL) | |
| | | 2014 NCT02349698 Phase 1/Phase | |
| | | 2 A Clinical Research of CAR T Cells | |
| | | Targeting CD19 Positive Malignant | |
| | | B-cell Derived Leukemia and | |
| | | Lymphoma | |
| Sunshine Lake | 2019 WO2020114358 DONG, | | Sunshine Lake |
| Pharma patent | Junji, CHEN . . . CD19 ANTIBODY | | Pharma |
| anti-CD19 | AND USES THEREOF | | |
| Systimmune | 2021 WO2021178253 | | Sichuan Baili |
| patent anti-CD19 | CHATTERJEE, Soumi . . . ANTI- | | Pharma |
| | CD19 ANTIBODIES AND | | Systimmune |
| | METHODS OF USING AND | | |
| | MAKING THEREOF | | |
| tafasitamab-cxix | 2022 US20220283166 ENDELL, | 2023 NCT05453500 Phase 2 | Incyte |
| | Jan; WIND . . . METHODS FOR | Chemotherapy (DA-EPOCH +/− R) and | Morphosys |
| | PREDICTING THERAPEUTIC | Targeted Therapy (Tafasitamab) for | Xencor |
| | BENEFIT OF ANTI-CD19 | the Treatment of Newly-Diagnosed | |
| | THERAPY IN PATIENTS | Philadelphia Chromosome Negative | |
| | 2022 US20220213190 | B Acute Lymphoblastic Leukemia | |
| | GARIDEL, Patrick; . . . ANTI-CD19 | 2022 NCT05455697 Phase 1/Phase | |
| | ANTIBODY FORMULATIONS | 2 Tafasitamab, Retifanlimab, and | |
| | 2021 US20220088197 ENDELL, | Rituximab in Combination With | |
| | Jan; WIND . . . COMBINATIONS | Chemotherapy (Cyclophosphamide, | |
| | AND USES THEREOF | Doxorubicin, Vincristine, and | |
| | 2021 WO2022117799 | Prednisone) for the Treatment of | |
| | FINGERLE-ROWSON, . . . ANTI- | Diffuse Large B-cell Lymphoma | |
| | CD19 COMBINATION THERAPY | 2022 NCT05366218 Phase 1/Phase | |
| | 2021 US20220184208 | 2 Tafasitamab (MOR00208) in | |
| | FINGERLE-ROWSON, . . . ANTI- | Pediatric Patients With Relapsed or | |
| | CD19 COMBINATION THERAPY | Refractory Acute B Lineage | |
| | 2021 WO2022115762 | Leukemia | |
| | LANGMUIR, Peter | 2022 NCT05205252 Phase 1/Phase | |
| | COMBINATION THERAPY WITH | 2 Multi Cohort Study of | |
| | AN ANTI-CD19 ANTIBODY AND | Tazemetostat in Combination With | |
| | PARSACLISIB | Various Treatments For R/R | |
| | 2021 WO2021259902 ENDELL, | Hematologic Malignancies | |
| | Jan, FING . . . ANTI-TUMOR | 2022 NCT05222555 Phase 1/Phase | |
| | COMBINATION THERAPY | 2 Safety and Pharmacokinetics | |
| | COMPRISING ANTI-CD19 | Study of a Modified Tafasitamab IV | |
| | ANTIBODY AND POLYPEPTIDES | Dosing Regimen Combined With | |
| | BLOCKING THE SIRPA-CD47 | Lenalidomide in R-R DLBCL Patients | |
| | INNATE IMMUNE CHECKPOINT | 2021 NCT04978584 Phase 2 | |
| | 2021 US20230014026 ENDELL, | Rituximab, Lenalidomide, | |
| | Jan; FING . . . Anti-Tumor | Acalabrutinib, Tafasitamab Alone | |
| | Combination Therapy | and With Combination | |
| | comprising Anti-CD19 Antibody | Chemotherapy for the Treatment of | |
| | and Polypeptides Blocking the | Newly Diagnosed Non-germinal | |
| | SIRPalpha-CD47 Innate | Center Diffuse Large B-Cell | |
| | Immune Checkpoint | Lymphoma, Smart Stop Study | |
| | 2021 WO2022115120 | 2021 NCT04809467 Phase 1/Phase | |
| | LANGMUIR, Peter | 2 A Study Evaluating Safety, PK, and | |
| | COMBINATION THERAPY WITH | Efficacy of Tafasitamab and | |
| | AN ANTI-CD19 ANTIBODY AND | Parsaclisib in Participants With | |
| | PARSACLISIB | Relapsed/Refractory Non Hodgkin | |
| | 2020 US20210130461 Endell, | Lymphoma (R/R NHL) or Chronic | |
| | Jan; Boxh . . . ANTI-TUMOR | Lymphocytic Leukemia (CLL) | |
| | COMBINATION THERAPY | 2021 NCT04824092 Phase 3 | |
| | COMPRISING ANTI-CD19 | Tafasitamab + Lenalidomide + R- | |
| | ANTIBODY AND GAMMA DELTA | CHOP Versus R-CHOP in Newly | |
| | T-CELLS | Diagnosed High-intermediate and | |
| | 2020 US20210130460 | High Risk DLBCL Patients | |
| | Ambarkhane, Sumee . . . ANTI- | 2020 NCT04697160 Observational | |
| | CD19 THERAPY IN | Retrospective Cohort Study of | |
| | COMBINATION WITH | Systemic Therapies for R/R DLBCL | |
| | LENALIDOMIDE FOR THE | 2020 NCT04300803 Expanded | |
| | TREATMENT OF LEUKEMIA OR | Access Program for Tafasitamab | |
| | LYMPHOMA | (MOR00208) in R/R DLBCL | |
| | 2020 WO2021084063 ENDELL, | 2019 NCT04134936 Phase 1 Phase | |
| | Jan, BOXH . . . ANTI-TUMOR | Ib Study to Assess Safety and | |
| | COMBINATION THERAPY | Preliminary Efficacy of Tafasitamab | |
| | COMPRISING ANTI-CD19 | or Tafasitamab Plus Lenalidomide in | |
| | ANTIBODY AND GAMMA DELTA | Addition to R-CHOP in Patients With | |
| | T-CELLS | Newly Diagnosed DLBCL | |
| | 2020 WO2021084062 | 2016 NCT02763319 Phase 2/Phase | |
| | AMBARKHANE, Sumee . . . ANTI- | 3 A Trial to Evaluate the Efficacy | |
| | CD19 THERAPY IN | and Safety of MOR208 With | |
| | COMBINATION WITH | Bendamustine (BEN) Versus | |
| | LENALIDOMIDE FOR THE | Rituximab (RTX) With BEN in Adult | |
| | TREATMENT OF LEUKEMIA OR | Patients With Relapsed or | |
| | LYMPHOMA | Refractory Diffuse Large B-cel | |
| | 2020 WO2021084064 ENDELL, | Lymphoma (DLBCL) | |
| | Jan, FRIC . . . SEQUENTIAL ANTI- | 2016 NCT02639910 Phase 2 Study | |
| | CD19 THERAPY | to Evaluate Efficacy and Safety of | |
| | 2020 WO2020225196 KUFFER, | MOR208 With Idelalisib in R/R | |
| | Christian . . . ANTI-CD19 | CLL/SLL Patients Pretreated With | |
| | THERAPY IN PATIENTS HAVING | BTKi | |
| | A LIMITED NUMBER OF NK | 2015 NCT02399085 Phase 2 A Study | |
| | CELLS | to Evaluate the Safety and Efficacy | |
| | 2020 US20200331998 PERTEL, | of Lenalidomide With MOR00208 in | |
| | Thomas Ch . . . ANTIBODIES | Patients With R-R DLBCL | |
| | AGAINST 4G7-DERIVED | 2013 NCT02005289 Phase 2 Anti- | |
| | CHIMERIC ANTIGEN | CD19 Monoclonal Antibody | |
| | RECEPTORS | XmAb557 and Lenalidomide in | |
| | 2020 WO2020214937 PERTEL, | Treating Patients With Relapsed, | |
| | Thomas Ch . . . ANTIBODIES | Refractory, or Previously Untreated | |
| | AGAINST 4G7-DERIVED | Chronic Lymphocytic Leukemia, | |
| | CHIMERIC ANTIGEN | Small Lymphocytic Lymphoma or | |
| | RECEPTORS | Prolymphocytic Leukemia | |
| | 2020 US20200353077 | 2012 NCT01685008 Phase 2 Study | |
| | AMERSDORFFER, Jut . . . | of Fc-Optimized Anti-CD19 | |
| | COMBINATIONS AND USES | Antibody (MOR00208) to Treat | |
| | THEREOF | Non-Hodgkin's Lymphoma (NHL) | |
| | 2020 US20200352975 | 2012 NCT01685021 Phase 2 Study | |
| | AMERSDORFFER, Jut . . . | of Fc-Optimized Anti-CD19 | |
| | COMBINATIONS AND USES | Antibody (MOR00208) to Treat B- | |
| | THEREOF | cell Acute Lymphoblastic | |
| | 2020 US20200317804 Bernett, | Leukemia(B-All) | |
| | Matthew . . . OPTIMIZED | 2010 NCT01161511 Phase 1 Safety | |
| | ANTIBODIES THAT TARGET | and Tolerability of XmAbÂ ®5574 in | |
| | CD19 | Chronic Lymphocytic Leukemia | |
| | 2020 US20200206228 ENDELL, | 2023 Nedved A, Maddock . . . Clinical | |
| | Jan; BOXH . . . COMBINATION OF | Treatment Guidelines for | |
| | AN ANTI-CD19 ANTIBODY AND | Tafasitamab Plus Lenalidomide in | |
| | A BRUTON'S TYROSINE KINASE | Patients with Relapsed or | |
| | INHIBITOR AND USES THEREOF | Refractory Diffuse Large B-Cell | |
| | 2018 WO2018220040 | Lymphoma. | |
| | KELEMEN, Peter, S . . . | 2022 Belada D, Kopecko . . . ABCL-022 | |
| | TREATMENT PARADIGM FOR | Pharmacokinetics and | |
| | AN ANTI-CD19 ANTIBODY AND | Pharmacodynamics in First-MIND: A | |
| | VENETOCLAX COMBINATION | Phase Ib, Open-Label, Randomized | |
| | TREATMENT | Study of Tafasitamab ± | |
| | 2018 US20210292410 | Lenalidomide + R-CHOP in Patients | |
| | Kelemen, Peter; S . . . | With Newly Diagnosed Diffuse | |
| | TREATMENT PARADIGM FOR | Large B-Cell Lymphoma. | |
| | AN ANTI-CD19 ANTIBODY AND | 2022 Vitolo U, Nowakow . . . ABCL- | |
| | VENETOCLAX COMBINATION | 023 frontMIND: A Phase III, | |
| | TREATMENT | Randomized, Double-Blind Study of | |
| | 2017 WO2018078123 ENDELL, | Tafasitamab + Lenalidomide + R- | |
| | Jan, PETR . . . COMBINATION OF | CHOP Versus R-CHOP Alone for | |
| | ANTI CD19 ANTIBODY WITH A | Newly Diagnosed High-Intermediate | |
| | BCL-2 INHIBITOR AND USES | and High-Risk Diffuse Large B-Cell | |
| | THEREOF | Lymphoma. | |
| | 2017 US20190241656 Endell, | 2022 Nowakowski G, Yoo . . . ABCL- | |
| | Jan; Petr . . . COMBINATION OF | 088 Subgroup Analysis in RE- | |
| | ANTI CD19 ANTIBODY WITH A | MIND2: An Observational, | |
| | BCL-2 INHIBITOR AND USES | Retrospective Cohort Study of | |
| | THEREOF | Tafasitamab + Lenalidomide Versus | |
| | 2017 US20180037653 Foster, | Systemic Therapies in Patients With | |
| | Paul; Byr . . . TREATMENT FOR | Relapsed/Refractory Diffuse Large | |
| | CHRONIC LYMPHOCYTIC | B-Cell Lymphoma. | |
| | LEUKEMIA (CLL) | 2022 Sehn L H, Hubel K, . . . IBCL-123 | |
| | 2017 WO2018002031 GARIDEL, | inMIND: A Phase 3 Study of | |
| | Patrick, . . . ANTI-CD19 | Tafasitamab Plus Lenalidomide and | |
| | ANTIBODY FORMULATIONS | Rituximab Versus Placebo Plus | |
| | 2017 US20190322742 Garidel, | Lenalidomide and Rituximab for | |
| | Patrick; . . . ANTI-CD19 | Relapsed/Refractory (R/R) Follicular | |
| | ANTIBODY FORMULATIONS | Lymphoma (FL) or Marginal Zone | |
| | 2017 U.S. Pat. No. 11,352,423 Garidel, | Lymphoma (MZL). | |
| | Patrick . . . Anti-CD19 antibody | 2022 Greil R, Kopeckov . . . ABCL-052 | |
| | formulations | MINDway: A Phase Ib/II Dose | |
| | 2017 WO2017214452 ZACK, | Optimization Study to Assess Safety | |
| | Debra, STON . . . TREATMENT OF | and Pharmacokinetics of | |
| | IGG4-RELATED DISEASES WITH | Tafasitamab + Lenalidomide in | |
| | ANTI-CD19 ANTIBODIES | Patients With Relapsed/Refractory | |
| | CROSSBINDING TO CD32B | Diffuse Large B-Cell Lymphoma. | |
| | 2017 US20190195879 Endell, | 2022 Duell J, Jurczak . . . ABCL-388 L- | |
| | Jan; Wind . . . METHODS FOR | MIND: Safety and Efficacy of | |
| | PREDICTING THERAPEUTIC | Tafasitamab in Patients With | |
| | BENEFIT OF ANTI-CD19 | Relapsed/Refractory Diffuse Large | |
| | THERAPY IN PATIENTS | B-Cell Lymphoma on Treatment for | |
| | 2016 WO2017032679 ENDELL, | at Least 2 Years. | |
| | Jan, WIND . . . COMBINATIONS | 2022 Nowakowski G S, Yo . . . | |
| | AND USES THEREOF | Improved Efficacy of Tafasitamab | |
| | 2016 US20180228893 Endell, | plus Lenalidomide versus Systemic | |
| | Jan; Wind . . . COMBINATIONS | Therapies for Relapsed/Refractory | |
| | AND USES THEREOF | DLBCL: RE-MIND2, an Observational | |
| | 2016 U.S. Pat. No. 11,224,654 Endell, Jan | Retrospective Matched Cohort | |
| | (Muni . . . Combinations and uses | Study. | |
| | thereof | 2022 Cherng H J, Westin JR | |
| | 2016 WO2016189014 ENDELL, | Broadening the MIND: Tafasitamab | |
| | Jan, BOXH . . . COMBINATION OF | and Lenalidomide versus Synthetic | |
| | AN ANTI-CD19 ANTIBODY AND | Controls. | |
| | A BRUTON'S TYROSINE KINASE | 2022 Moore D C, Eagers . . . | |
| | INHIBITOR AND USES THEREOF | Tafasitamab and lenalidomide for | |
| | 2016 US20180153892 Endell, | relapsed/refractory diffuse large B- | |
| | Jan; Boxh . . . COMBINATION OF | cell lymphoma in a patient on | |
| | AN ANTI-CD19 ANTIBODY AND | chronic intermittent hemodialysis. | |
| | A BRUTON'S TYROSINE KINASE | 2022 Her J H, Pretscher . . . | |
| | INHIBITOR AND USES THEREOF | Tafasitamab mediates killing of B- | |
| | 2016 U.S. Pat. No. 10,617,691 Endell, Jan | cell non-Hodgkin's lymphoma in | |
| | (Muni . . . Combination of an | combination with γδ T cell or | |
| | anti-CD19 antibody and a | allogeneic NK cell therapy. | |
| | Bruton's tyrosine kinase | 2022 Serrano P, Yuen H . . . Real- | |
| | inhibitor and uses thereof | world data in drug development | |
| | 2015 WO2015195498 FOSTER, | strategies for orphan drugs: | |
| | Paul, BY . . . TREATMENT FOR | Tafasitamab in B-cell lymphoma, a | |
| | CHRONIC LYMPHOCYTIC | case study for an approval based on | |
| | LEUKEMIA (CLL) | a single-arm combination trial. | |
| | 2015 US20170137516 Foster, | 2021 Delgado J, Papado . . . The | |
| | Paul; Byr . . . TREATMENT FOR | European Medicines Agency Review | |
| | CHRONIC LYMPHOCYTIC | of Tafasitamab in Combination With | |
| | LEUKEMIA (CLL) | Lenalidomide for the Treatment of | |
| | 2014 WO2014184143 | Adult Patients With | |
| | GALETTO, Roman, S . . . CD19 | Relapsed/Refractory Diffuse Large | |
| | SPECIFIC CHIMERIC ANTIGEN | B-cell Lymphoma. | |
| | RECEPTOR AND USES THEREOF | 2021 Tilch M K, Robak T . . . Safety of | |
| | 2013 US20140112916 Bernett, | the Anti-CD19 antibody | |
| | Matthew . . . Optimized | Tafasitamab in Long Term | |
| | Antibodies That Target CD19 | Responders from A Phase II Trial for | |
| | 2013 US20140086906 Bernett, | Relapsed Lymphoma. | |
| | Matthew . . . Optimized | 2021 Zinzani P L, Rodge . . . RE-MIND: | |
| | Antibodies That Target CD19 | Comparing tafasitamab + | |
| | 2013 U.S. Pat. No. 9,803,020 Bernett, | lenalidomide (L-MIND) with a | |
| | Matthew . . . Optimized | real-world lenalidomide | |
| | antibodies that target CD19 | monotherapy cohort in relapsed or | |
| | 2013 U.S. Pat. No. 8,858,937 Lazar, | refractory diffuse large B-cell | |
| | Gregory Al . . . Optimized Fc | lymphoma. | |
| | variants and methods for their | 2021 Staber P B, Jurcza . . . | |
| | generation | Tafasitamab combined with | |
| | 2012 WO2013024097 | idelalisib or venetoclax in patients | |
| | AMERSDORFER, Jutt . . . | with CLL previously treated with a | |
| | COMBINATION THERAPY WITH | BTK inhibitor. | |
| | AN ANTI - CD19 ANTIBODY AND | 2021 Klisovic R B, Leun . . . A phase 2a, | |
| | A NITROGEN MUSTARD | single-arm, open-label study of | |
| | 2012 WO2013024095 | tafasitamab, a humanized, Fc- | |
| | AMERSDORFFER, Jut . . . | modified, anti-CD19 antibody, in | |
| | COMBINATION THERAPY WITH | patients with relapsed/refractory B- | |
| | AN ANTI - CD19 ANTIBODY AND | precursor cell acute lymphoblastic | |
| | A PURINE ANALOG | leukemia. | |
| | 2012 US20140227277 | 2021 Duell J, Maddocks . . . Long- | |
| | Amersdorffer, Jut . . . | term outcomes from the Phase II L- | |
| | Combination therapy with an | MIND study of tafasitamab | |
| | anti-CD19 antibody and a | (MOR208) plus lenalidomide in | |
| | purine analog | patients with relapsed or refractory | |
| | 2012 US20140255427 | diffuse large B-cell lymphoma. | |
| | Amersdorffer, Jut . . . | 2020 Roßkopf S, Eichho . . . Enhancing | |
| | COMBINATION THERAPY WITH | CDC and ADCC of CD19 Antibodies | |
| | AN ANTI - CD19 ANTIBODY AND | by Combining Fc Protein- | |
| | A NITROGEN MUSTARD | Engineering with Fc Glyco- | |
| | 2011 US20120082664 Bernett, | Engineering. | |
| | Matthew . . . OPTIMIZED | 2020 Hoy S M Tafasitamab: First | |
| | ANTIBODIES THAT TARGET | Approval. | |
| | CD19 | 2020 Salles G, Duell J . . . Tafasitamab | |
| | 2007 WO2008022152 | plus lenalidomide in relapsed or | |
| | BERNETT, Matthew . . . | refractory diffuse large B-cell | |
| | OPTIMIZED ANTIBODIES THAT | lymphoma (L-MIND): a multicentre, | |
| | TARGET CD19 | prospective, single-arm, phase 2 | |
| | 2007 US20100272723 Bernett, | study. | |
| | Matthew . . . Optimized | 2016 Seidel U J, Schleg . . . Reduction | |
| | Antibodies that Target CD19 | of minimal residual disease in | |
| | 2007 US20080260731 Bernett, | pediatric B-lineage acute | |
| | Matthew . . . OPTIMIZED | lymphoblastic leukemia by an Fc- | |
| | ANTIBODIES THAT TARGET | optimized CD19 antibody. | |
| | CD19 | 2016 Jurczak W, Bryk A . . . Single- | |
| | 2007 U.S. Pat. No. 8,524,867 Bernett, | agent MOR208 salvage and | |
| | Matthew . . . Optimized | maintenance therapy in a patient | |
| | antibodies that target CD19 | with refractory/relapsing diffuse | |
| | | large B-cell lymphoma: a case | |
| | | report. | |
| | | 2014 Woyach J A, Awan F . . . A phase | |
| | | I trial of the Fc engineered CD19 | |
| | | antibody XmAb ®5574 (MOR00208) | |
| | | demonstrates safety and | |
| | | preliminary efficacy in relapsed | |
| | | chronic lymphocytic leukemia. | |
| | | 2013 Kellner C, Zhukov . . . The Fc- | |
| | | engineered CD19 antibody MOR208 | |
| | | (XmAb5574) induces natural killer | |
| | | cell-mediated lysis of acute | |
| | | lymphoblastic leukemia cells from | |
| | | pediatric and adult patients. | |
| | | 2012 Rafiq S, Cheney C . . . XmAb- | |
| | | 5574 antibody demonstrates | |
| | | superior antibody dependent | |
| | | cellular cytotoxicity as compared to | |
| | | CD52 and CD20 targeted antibodies | |
| | | in adult acute lymphoblastic | |
| | | leukemia cells. | |
| | | 2008 Zalevsky J, Leung . . . The impact | |
| | | of Fc engineering on an anti-CD19 | |
| | | antibody: increased Fcgamma | |
| | | receptor affinity enhances B-cell | |
| | | clearing in nonhuman primates. | |
| | | 2008 Horton H M, Bernet . . . Potent | |
| | | in vitro and in vivo activity of an Fc- | |
| | | engineered anti-CD19 monoclonal | |
| | | antibody against lymphoma and | |
| | | leukemia. | |
| | | ASCO 2012 Evaluation of | |
| | | combination therapies with | |
| | | MOR00208, an Fc-enhanced | |
| | | humanized CD19 antibody, in | |
| | | models of lymphoma Mark | |
| | | Winderlich, . . . | |
| | | ASCO 2016 Subgroup analyses of | |
| | | diffuse large B-cell lymphoma | |
| | | (DLBCL) and indolent lymphoma | |
| | | cohorts from a phase IIa study of | |
| | | single-agent MOR208 in patients | |
| | | with relapsed or refractory non- | |
| | | Hodgkin's lymphoma (R-R NHL) | |
| | | Wojciech Jurczak, . . . | |
| | | ASCO 2016 A phase II study of | |
| | | MOR208 plus idelalisib in patients | |
| | | with relapsed or refractory chronic | |
| | | lymphocytic leukemia (CLL) or small | |
| | | lymphocytic lymphoma (SLL) | |
| | | previously treated with a Bruton's | |
| | | tyrosine kinase inhibitor (MIRACLE) | |
| | | Clemens-Martin We . . . | |
| | | ASCO 2017 B-MIND: MOR208 plus | |
| | | bendamustine (BEN) versus | |
| | | rituximab (RTX) plus BEN in patients | |
| | | with relapsed or refractory (R-R) | |
| | | diffuse large B-cell lymphoma | |
| | | (DLBCL): An open-label, randomized | |
| | | phase II/III trial. Grzegorz S. | |
| | | Nowak . . . | |
| | | ASH 2012 Use of a Fc-Optimized | |
| | | CD19 Antibody for Treatment of | |
| | | MRD in Pediatric Patients with B- | |
| | | Lineage Acute Lymphoblastic | |
| | | Leukemia Peter Lang, MD, H . . . | |
| | | ASH 2012 The CD19 Antibody | |
| | | MOR208 Efficiently Triggers Natural | |
| | | Killer Cell-Mediated Cytotoxicity | |
| | | Against Acute Lymphoblastic | |
| | | Leukemia Cells From Pediatric and | |
| | | Adult Patients Christian Kellner . . . | |
| | | ASH 2013 From Bedside To Bench: | |
| | | Molecular Benchmarking Of An | |
| | | Fc-Optimized CD19 Antibody Used | |
| | | In Treatment Of Relapsed and | |
| | | Refractory Pediatric B-Lineage | |
| | | Acute Lymphoblastic Leukemia | |
| | | Ursula J. E. Seide . . . | |
| | | ASH 2014 Final Results and Follow- | |
| | | up of a Phase I Study of the Fc | |
| | | Engineered CD19 Antibody | |
| | | XmAb ®5574 (aka MOR00208) in | |
| | | Patients with Relapsed or | |
| | | Refractory Chronic Lymphocytic | |
| | | Leukemia (CLL) or Small | |
| | | Lymphocytic Lymphoma (SLL) | |
| | | Jennifer A. Woyac . . . | |
| | | ASH 2015 A Phase II Study of the Fc | |
| | | Engineered CD19 Antibody MOR208 | |
| | | in Combination with Lenalidomide | |
| | | for Patients with Chronic | |
| | | Lymphocytic Leukemia (CLL) | |
| | | Jennifer A. Woyac . . . | |
| | | ASH 2016 Updated Results from a | |
| | | Phase II Study of the Fc Engineered | |
| | | CD19 Antibody MOR208 in | |
| | | Combination with Lenalidomide for | |
| | | Patients with Chronic Lymphocytic | |
| | | Leukemia (CLL) and Richter's | |
| | | Transformation or Ibrutinib for | |
| | | Patients with Ibrutinib-Resistant | |
| | | Clones Jennifer A. Woyac . . . | |
| | | ASH 2019 Targeting of CD19 By | |
| | | Tafasitamab Does Not Impair CD19 | |
| | | Directed Chimeric Antigen Receptor | |
| | | T Cell Activity in Vitro Paulina | |
| | | Horvei, M . . . | |
| taplitumomab | | 2000 NCT00004858 Phase I | NCI |
| paptox | | Monoclonal Antibody Therapy in | |
| | | Treating Patients With Recurrent | |
| | | Acute Lymphoblastic Leukemia or | |
| | | Non-Hodgkin's Lymphoma | |
| Teneobio patent | 2022 WO2022216864 | | TeneoBio |
| anti-CD19 | TRINKLEIN, Nathan ANTI-CD19 | | |
| | ANTIBODIES AND CAR-T | | |
| | STRUCTURES | | |
| | 2019 WO2020018922 ALDRED, | | |
| | Shelley F . . . HEAVY CHAIN | | |
| | ANTIBODIES BINDING TO CD19 | | |
| | 2019 US20210332133 Force | | |
| | Aldred, She . . . HEAVY CHAIN | | |
| | ANTIBODIES BINDING TO CD19 | | |
| tisagenlecleucel | 2022 WO2022254337 ENGELS, | 2022 NCT05310591 Phase 1/Phase | Novartis |
| | Boris CD19 AND CD22 | 2 Combination of an Anti-PD1 | U. Penn. |
| | CHIMERIC ANTIGEN | Antibody With Tisagenlecleucel | |
| | RECEPTORS AND USES | Reinfusion in Children, Adolescents | |
| | THEREOF | and Young Adults With Acute | |
| | 2020 WO2021108613 ENGELS, | Lymphoblastic Leukemia After Loss | |
| | Boris, G U . . . CD19 AND CD22 | of Persistence | |
| | CHIMERIC ANTIGEN | 2021 NCT04456023 Phase 2 Study | |
| | RECEPTORS AND USES | of Tisagenlecleucel in Chinese Adult | |
| | THEREOF | Patients With Relapsed or | |
| | 2019 WO2020069409 ISAACS, | Refractory Diffuse Large B-cell Non- | |
| | Randi, FR . . . CD19 CHIMERIC | Hodgkin Lymphoma (DLBCL) | |
| | ANTIGEN RECEPTOR (CAR) AND | 2019 NCT03628053 Phase 3 | |
| | CD22 CAR COMBINATION | Tisagenlecleucel vs Blinatumomab | |
| | THERAPIES | or Inotuzumab for Patients With | |
| | 2019 WO2019210153 NOBLES, | Relapsed/Refractory B-cell | |
| | Christoph . . . CAR T CELL | Precursor Acute Lymphoblastic | |
| | THERAPIES WITH ENHANCED | Leukemia | |
| | EFFICACY | 2018 NCT03630159 Phase 1 Study | |
| | 2019 U.S. Pat. No. 11,149,076 Bitter, Hans | of Tisagenlecleucel in Combination | |
| | (Lin . . . CD20 therapies, CD22 | With Pembrolizumab in r/r Diffuse | |
| | therapies, and combination | Large B-cell Lymphoma Patients | |
| | therapies with a CD19 chimeric | 2018 NCT03568461 Phase 2 Efficacy | |
| | antigen receptor (CAR)- | and Safety of Tisagenlecleucel in | |
| | expressing cell | Adult Patients With Refractory or | |
| | 2018 WO2019099639 | Relapsed Follicular Lymphoma | |
| | GARFALL, Alfred, . . . BCMA- | 2018 NCT03610724 Phase 2 Phase II | |
| | TARGETING CHIMERIC | Open Label Trial to Determine | |
| | ANTIGEN RECEPTOR, CD19- | Safety & Efficacy of Tisagenlecleucel | |
| | TARGETING CHIMERIC | in Pediatric Non-Hodgkin | |
| | ANTIGEN RECEPTOR, AND | Lymphoma Patients | |
| | COMBINATION THERAPIES | 2017 NCT03118180 Phase 1/Phase | |
| | 2018 US20180271907 June, | 2 CD19 Targeted Chimeric Antigen | |
| | Carl H.; Le . . . USE OF CART19 TO | Receptor T Cells for B Cell | |
| | DEPLETE NORMAL B CELLS TO | Lymphoma | |
| | INDUCE TOLERANCE | 2016 NCT03027739 Phase 2/Phase | |
| | 2017 US20180258391 June, | 3 CART-19 Cells For MRD Positive | |
| | Carl H.; Le . . . Compositions and | CD19+ ALL | |
| | Methods for Treatment of | 2016 NCT02935543 Phase 2 CART19 | |
| | Cancer | in Patient With ALL | |
| | 2017 WO2018023025 ANAK, | 2016 NCT02924753 Phase 1 The | |
| | Oezlem, BIL . . . COMBINATION | Safety and Efficacy of CART-19 Cells | |
| | THERAPIES OF CHIMERIC | in B-cell Acute Lymphoblastic | |
| | ANTIGEN RECEPTORS ADN PD-1 | Leukemia (B-ALL). | |
| | INHIBITORS | 2016 NCT03101709 Phase 1 The | |
| | 2016 US20170283775 June, | Safety and Efficacy of CART-19 Cells | |
| | Carl H.; Le . . . Compositions and | in Relapse and Refractory Patients | |
| | Methods for Treatment of | With CD19+ B-cell Lymphoma | |
| | Cancer | 2016 NCT02906371 N/A Study of | |
| | 2016 WO2016164731 BITTER, | the Tocilizumab Optimization | |
| | Hans, BOR . . . CD20 THERAPIES, | Timing for CART19 Associated | |
| | CD22 THERAPIES, AND | Cytokine Release Syndrome | |
| | COMBINATION THERAPIES | 2016 NCT02167360 Phase 2 Study | |
| | WITH A CD19 CHIMERIC | of Efficacy and Safety of CTL019 in | |
| | ANTIGEN RECEPTOR (CAR) - | Adult ALL Patients | |
| | EXPRESSING CELL | 2016 NCT02794246 Phase 2 CART- | |
| | 2016 US20160362472 Bitter, | 19 Post-ASCT for Multiple Myeloma | |
| | Hans; Bor . . . CD20 THERAPIES, | 2016 NCT02810223 Phase 1 Efficacy | |
| | CD22 THERAPIES, AND | of CART-19 Cell Therapy in B Cell | |
| | COMBINATION THERAPIES | Acute Lymphoblastic Leukemia | |
| | WITH A CD19 CHIMERIC | 2016 NCT02650999 Phase 1/Phase | |
| | ANTIGEN RECEPTOR (CAR)- | 2 Study of Pembrolizumab in | |
| | EXPRESSING CELL | Patients Failing to Respond to or | |
| | 2016 U.S. Pat. No. 10,253,086 Bitter, Hans | Relapsing After Anti-CD19 Chimeric | |
| | (Cam . . . CD20 therapies, CD22 | Antigen Receptor Modified T Cell | |
| | therapies, and combination | Therapy for Relapsed or Refractory | |
| | therapies with a CD19 chimeric | CD19+ Lymphomas | |
| | antigen receptor (CAR)- | 2015 NCT02640209 N/A Pilot Trial | |
| | expressing cell | Of Autologous T Cells Engineered To | |
| | 2016 WO2016164580 ANGST, | Express Anti-CD19 Chimeric Antigen | |
| | Daniela, B . . . COMBINATION OF | Receptor (CART19)In Combination | |
| | CHIMERIC ANTIGEN RECEPTOR | With Ibrutinib In Patients With | |
| | THERAPY AND AMINO | Relapsed Or Refractory CD19+ | |
| | PYRIMIDINE DERIVATIVES | Chronic Lymphocytic Leukemia | |
| | 2016 US20160130355 June, | (CLL)Or Small Lymphocytic | |
| | Carl H.; Le . . . Compositions and | Lymphoma (SLL) | |
| | Methods for Treatment of | 2015 NCT02624258 Early Phase 1 | |
| | Cancer | Pilot Study of Non-Viral, RNA- | |
| | 2016 US20160194404 June, | Redirected Autologous T Cells in | |
| | Carl H.; Le . . . Compositions and | Patients With Refractory or | |
| | Methods for Treatment of | Relapsed Hodgkin Lymphoma | |
| | Cancer | 2015 NCT02799550 Phase 1 | |
| | 2016 US20160208012 June, | Allogeneic CART-19 for Elderly | |
| | Carl H.; Le . . . Compositions and | Relapsed/Refractory CD19+ ALL | |
| | Methods for Treatment of | 2015 NCT02445248 Phase 2 Study | |
| | Cancer | of Efficacy and Safety of CTL019 in | |
| | 2016 U.S. Pat. No. 9,464,140 June, Carl H.; | Adult DLBCL Patients | |
| | Le . . . Compositions and | 2015 NCT02445222 N/A CD19 CART | |
| | methods for treatment of | Long Term Follow Up (LTFU) Study | |
| | cancer | 2015 NCT02813837 N/A Chimeric | |
| | 2016 U.S. Pat. No. 9,518,123 June, Carl H.; | Antigen Receptor T Cells (CART) | |
| | Le . . . Compositions and | Therapy in Refractory/Relapsed B | |
| | methods for treatment of | Cell Hematologic Malignancies | |
| | cancer | 2015 NCT02435849 Phase 2 | |
| | 2016 U.S. Pat. No. 9,540,445 June, Carl H.; | Determine Efficacy and Safety of | |
| | Le . . . Compositions and | CTL019 in Pediatric Patients With | |
| | methods for treatment of | Relapsed and Refractory B-cell ALL | |
| | cancer | 2014 NCT02277522 Early Phase 1 | |
| | 2015 US20160159907 June, | CD19 Redirected Autologous T Cells | |
| | Carl H.; Le . . . Compositions and | for Hodgkin Lymphoma | |
| | Methods for Treatment of | 2014 NCT02228096 Phase 2 Study | |
| | Cancer | of Efficacy and Safety of CTL019 in | |
| | 2015 U.S. Pat. No. 9,481,728 June, Carl H.; | Pediatric ALL Patients | |
| | Le . . . Compositions and | 2014 NCT02476734 Early Phase 1 | |
| | methods for treatment of | FDG-PET/CT Imaging as Early | |
| | cancer | Predictor of DP | |
| | 2015 WO2016109410 BEDOYA, | 2014 NCT02135406 Phase 1 CART- | |
| | Felipe, G . . . METHODS OF | 19 for Multiple Myeloma | |
| | MAKING CHIMERIC ANTIGEN | 2014 NCT02374333 Phase 1 Pilot | |
| | RECEPTOR-EXPRESSING CELLS | Study of Redirected Autologous T | |
| | 2015 US20170335281 Loew, | Cells Engineered to Contain | |
| | Andreas; Mi . . . TREATMENT OF | Humanized Anti-CD19 in Patients | |
| | CANCER USING CHIMERIC | With Relapsed or Refractory CD19+ | |
| | ANTIGEN RECEPTOR | Leukemia and Lymphoma | |
| | 2014 US20140271635 Brogdon, | Previously Treated With Cell | |
| | Jennifer . . . TREATMENT OF | Therapy | |
| | CANCER USING HUMANIZED | 2014 NCT02030834 Phase 2 Phase | |
| | ANTI-CD19 CHIMERIC ANTIGEN | IIa Study of Redirected Autologous | |
| | RECEPTOR | T Cells Engineered to Contain Anti- | |
| | 2014 WO2014153270 | CD19 Attached to TCRz and 4- | |
| | BROGDON, Jennifer . . . | Signaling Domains in Patients With | |
| | TREATMENT OF CANCER USING | Chemotherapy Relapsed or | |
| | HUMANIZED ANTI-CD19 | Refractory CD19+ Lymphomas | |
| | CHIMERIC ANTIGEN RECEPTOR | 2014 NCT02030847 Phase 2 Study | |
| | 2013 U.S. Pat. No. 9,328,156 June, Carl H.; | of Redirected Autologous T Cells | |
| | Le . . . Use of chimeric antigen | Engineered to Contain Anti-CD19 | |
| | receptor-modified T cells to | Attached to TCR and 4-1BB | |
| | treat cancer | Signaling Domains in Patients With | |
| | 2013 US20140106449 June, | Chemotherapy Resistant or | |
| | Carl H.; Le . . . Use of Chimeric | Refractory Acute Lymphoblastic | |
| | Antigen Receptor-Modified T | Leukemia | |
| | Cells to Treat Cancer | 2012 NCT01747486 Phase 2 CD19 | |
| | 2011 U.S. Pat. No. 9,499,629 June, Carl H.; | Redirected Autologous T Cells | |
| | Le . . . Use of chimeric antigen | 2011 NCT01626495 Phase 1 Phase | |
| | receptor-modified T-cells to | I/IIA Study of CART19 Cells for | |
| | treat cancer | Patients With Chemotherapy | |
| | 2011 US20130287748 June, | Resistant or Refractory CD19+ | |
| | Carl H.; Le . . . Use of Chimeric | Leukemia and Lymphoma | |
| | Antigen Receptor-Modified T- | 2010 NCT01551043 Phase 1 Allo | |
| | Cells to Treat Cancer | CART-19 Protocol | |
| | 2011 WO2012079000 JUNE, | 2009 NCT01029366 Phase 1 CART19 | |
| | Carl, H., L . . . USE OF CHIMERIC | to Treat B-Cell Leukemia or | |
| | ANTIGEN RECEPTOR-MODIFIED | Lymphoma That Are Resistant or | |
| | T CELLS TO TREAT CANCER | Refractory to Chemotherapy | |
| | | NCT03123939 N/A Expanded | |
| | | Treatment Protocol in Acute | |
| | | Lymphoblastic Leukemia | |
| | | 2021 Mueller K T, Grupp . . . | |
| | | Tisagenlecleucel Immunogenicity in | |
| | | Relapsed/Refractory Acute | |
| | | Lymphoblastic Leukemia and | |
| | | Diffuse Large B-Cell Lymphoma. | |
| | | 2020 Thakur A, Scholle . . . Enhanced | |
| | | cytotoxicity against solid tumors by | |
| | | bispecific antibody-armed CD19 | |
| | | CAR T cells: a proof-of-concept | |
| | | study. | |
| | | 2018 Mueller K T, Waldr . . . Clinical | |
| | | Pharmacology of Tisagenlecleucel in | |
| | | B-Cell Acute Lymphoblastic | |
| | | Leukemia. | |
| | | 2017 Xue Q, Bettini E, . . . Single-cell | |
| | | multiplexed cytokine profiling of | |
| | | CD19 CAR-T cells reveals a diverse | |
| | | landscape of polyfunctional | |
| | | antigen-specific response. | |
| | | 2016 Bhoj V G, Arhontou . . . | |
| | | Persistence of long-lived plasma | |
| | | cells and humoral immunity in | |
| | | individuals responding to CD19- | |
| | | directed CAR T-cell therapy. | |
| | | ASCO 2016 Recovery of humoral | |
| | | immunity in patients with durable | |
| | | complete responses following | |
| | | chimeric antigen receptor modified | |
| | | t cells directed against CD19 | |
| | | (CTL019) Stephen J. Schust . . . | |
| U. Florida patent | 2016 US20180142034 Chang, | | U. Florida |
| anti-CD19 CAR | Lung-Ji; CHIMERIC ANTIGEN | | |
| | RECEPTORS AND USES | | |
| | THEREOF | | |
| | 2016 U.S. Pat. No. 11,248,058 Chang, Lung- | | |
| | Ji (G . . . Chimeric antigen | | |
| | receptors and uses thereof | | |
| U. Texas patent | 2018 US20180265595 COOPER, | 2019 Klesmith J R, Wu L . . . Fine | U. Texas |
| anti-CD19 | Laurence . . . ANTI-CD19 scFv | epitope mapping of the CD19 | |
| | (FMC63) POLYPEPTIDE AND | extracellular domain promotes | |
| | USES THEREOF | design. | |
| | 2017 US20170342164 COOPER, | | |
| | Laurence . . . ANTI-CD19 scFv | | |
| | (FMC63) POLYPEPTIDE | | |
| | 2010 WO2012057765 VITETTA, | | |
| | Ellen, S . . . RECOMBINANT ANTI- | | |
| | CD19 MONOCLONAL | | |
| | ANTIBODIES | | |
| | 2002 U.S. Pat. No. 8,658,168 Ghetie, | | |
| | Maria-Ana . . . Compositions and | | |
| | methods for homoconjugates | | |
| | of antibodies which induce | | |
| | growth arrest of apoptosis of | | |
| | tumor cells | | |
| UCART19 | 2020 US20210060079 | 2022 NCT05535855 Phase 1 UCD19 | Allogene |
| | GALETTO, Roman; S . . . CD19 | CAR T Therapy in Adults With B-ALL | Cellectis |
| | SPECIFIC CHIMERIC ANTIGEN | and MRD Positivity in CR1 | |
| | RECEPTOR AND USES THEREOF | 2016 NCT02735083 N/A A Study to | |
| | 2020 US20210060080 | Evaluate the Long-term Safety of | |
| | GALETTO, Roman; S . . . CD19 | Patients With Advanced Lymphoid | |
| | SPECIFIC CHIMERIC ANTIGEN | Malignancies Who Have Been | |
| | RECEPTOR AND USES THEREOF | Previously Administered With | |
| | 2020 U.S. Pat. No. 11,007,224 Galetto, | UCART19 | |
| | Roman (P . . . CD19 specific | 2016 NCT02746952 Phase 1 Dose | |
| | chimeric antigen receptor and | Escalation Study of UCART19 in | |
| | uses thereof | Adult Patients With Relapsed/ | |
| | 2020 U.S. Pat. No. 11,077,144 Galetto, | Refractory B-cell Acute | |
| | Roman (P . . . CD19 specific | Lymphoblastic Leukaemia | |
| | chimeric antigen receptor and | 2016 NCT02808442 Phase 1 Study | |
| | uses thereof | of UCART19 in Pediatric Patients | |
| | 2020 WO2020219848 PERTEL, | With Relapsed/Refractory B Acute | |
| | Thomas Ch . . . RITUXIMAB- | Lymphoblastic Leukemia | |
| | RESISTANT CHIMERIC ANTIGEN | | |
| | RECEPTORS AND USES | | |
| | THEREOF | | |
| | 2020 US20200384026 PERTEL, | | |
| | Thomas Ch . . . RITUXIMAB- | | |
| | RESISTANT CHIMERIC ANTIGEN | | |
| | RECEPTORS AND USES | | |
| | THEREOF | | |
| | 2019 ucarUS20190209616 | | |
| | CD19 SPECIFIC CHIMERIC | | |
| | ANTIGEN RECEPTOR AND USES | | |
| | THEREOF | | |
| | 2014 US20160145337 | | |
| | GALETTO, Roman; S . . . CD19 | | |
| | SPECIFIC CHIMERIC ANTIGEN | | |
| | RECEPTOR AND USES THEREOF | | |
| | 2014 U.S. Pat. No. 10,874,693 Galetto, | | |
| | Roman (P . . . CD19 specific | | |
| | chimeric antigen receptor and | | |
| | uses thereof | | |
| | US20210000869 Galetto, | | |
| | Roman; S . . . CD19 SPECIFIC | | |
| | CHIMERIC ANTIGEN RECEPTOR | | |
| | AND USES THEREOF | | |
| UCB patent anti- | 2021 WO2021224629 | | Autolus UCB |
| CD19 CAR | SRIVASTAVA, Saket . . . CELL | | |
| | 2019 WO2019220109 PULÉ, | | |
| | Martin, KOK . . . CHIMERIC | | |
| | ANTIGEN RECEPTOR | | |
| | 2015 WO2016102965 PULÉ, | | |
| | Martin, CO . . . CELL | | |
| UCL patent anti- | 2018 US20190038672 Pule, | | UCL |
| CD19 CAR | Martin; Kon . . . Cell | | |
| | 2016 WO2016139487 PULÉ, | | |
| | Martin, MEK . . . CHIMERIC | | |
| | ANTIGEN RECEPTOR (CAR) | | |
| | COMPRISING A CD19-BINDING | | |
| | DOMAIN | | |
| | 2014 US20160296562 Pule, | | |
| | Martin; Kon . . . CELL | | |
| Uppsala U. anti- | | 2014 NCT02132624 Phase 1/Phase | Uppsala U. |
| CD19 CAR | | 2 CD19-targeting 3rd Generation | |
| | | CAR T Cells for Refractory B Cell | |
| | | Malignancy - a Phase I/IIa Trial. | |
| | | 2015 Hillerdal V, Essa . . . Chimeric | |
| | | antigen receptor-engineered T cells | |
| | | for the treatment of metastatic | |
| | | prostate cancer. | |
| Uwell Bio patent | 2018 WO2019119822 PANG, | | Uwell Bio |
| anti-CD19 CAR | Te-Ling, KO . . . | | |
| | PHARMACEUTICAL CHIMERIC | | |
| | RECEPTOR COMPOSITION AND | | |
| | METHOD THEREOF | | |
| VB119 | | 2021 NCT04652570 Phase 1/Phase | Cancer Res UK |
| | | 2 Efficacy and Safety of VB119 in | ValenzaBio |
| | | Subjects With Membranous | |
| | | Nephropathy | |
| WuXi Biologics | 2018 WO2019057100 LI, Jing, | | WuXi Biologics |
| patent anti-CD19 | LIU, Ji . . . NOVEL ANTI-CD19 | | |
| | ANTIBODIES | | |
| | 2018 US20200289563 LI, Jing; | | |
| | LIU, Ji . . . NOVEL ANTI-CD19 | | |
| | ANTIBODIES | | |
| | 2018 U.S. Pat. No. 11,497,769 Li, Jing; Liu, | | |
| | Ji . . . Anti-CD19 antibodies | | |
| Zhejiang Ruijiamei | 2021 WO2021213235 YANG, | | Zhejiang |
| Bio patent anti- | Liming, WAN . . . TECHNIQUE | | Ruijiamei Bio |
| CD19 | FOR PREPARING UNIVERSAL | | |
| | HUMANISED CAR19-DNT CELLS | | |
| | AND APPLICATION THEREFOR | | |
| | 2019 WO2020108090 WU, | | |
| | Lijun, GOLUBO . . . CAR-T CELLS | | |
| | WITH HUMANIZED CD19 SCFV | | |
| | WITH MUTATION IN CDR 1 | | |
| | REGION | | |
| A-319 | 2021 US20210393776 ZHOU, | 2019 NCT04056975 Phase 1 Study | Generon |
| | Hongxing; BISPECIFIC FAB | of A-319 in Patients With Relapsed | ITabMed |
| | FUSION PROTEINS | or Refractory B-cell Lymphoma | |
| | COMPRISING A CD3-BINDING | 2021 Wang S, Peng L, X . . . Preclinical | |
| | FAB FRAGMENT WITH N- | characterization and comparison | |
| | TERMINAL FUSION TO A | between CD3/CD19 bispecific and | |
| | BINDING DOMAIN AND | novel CD3/CD19/CD20 trispecific | |
| | METHODS OF USE | antibodies against B-cell acute | |
| | 2019 WO2020135335 HUANG, | lymphoblastic leukemia: targeted | |
| | Zhihua, SH . . . MULTISPECIFIC | immunotherapy for acute | |
| | ANTIGEN BINDING PROTEINS | lymphoblastic leukemia. | |
| | CAPABLE OF BINDING CD19 | | |
| | AND CD3, AND USE THEREOF | | |
| | 2019 US20220064295 HUANG, | | |
| | Zhihua; SH . . . MULTISPECIFIC | | |
| | ANTIGEN BINDING PROTEINS | | |
| | CAPABLE OF BINDING CD19 | | |
| | AND CD3, AND USE THEREOF | | |
| | 2019 WO2020048525 HUANG, | | |
| | Zhihua, RA . . . BISPECIFIC | | |
| | ANTIGEN BINDING PROTEINS | | |
| | AND USES THEREOF | | |
| | 2019 US20210301018 HUANG, | | |
| | Zhihua; RA . . . BISPECIFIC | | |
| | ANTIGEN BINDING PROTEINS | | |
| | AND USES THEREOF | | |
| | 2018 WO2020132810 HUANG, | | |
| | Zhihua, SH . . . MULTISPECIFIC | | |
| | ANTIGEN BINDING PROTEINS | | |
| | CAPABLE OF BINDING CD19 | | |
| | AND CD3, AND USE THEREOF | | |
| AFM11 | 2004 US20050079170 Le Gall; | 2016 NCT02848911 Phase 1 Safety | Affimed |
| | Fabrice; . . . Dimeric and | Study to Assess AFM11 in Patients | |
| | multimeric antigen binding | With Relapsed or Refractory Adult | |
| | structure | B-precursor ALL | |
| | 2002 WO2003025018 LE GALL, | 2014 NCT02106091 Phase 1 Safety | |
| | Fabrice, . . . DIMERIC AND | Study to Assess AFM11 in Patients | |
| | MULTIMERIC ANTIGEN | With Relapsed and/or Refractory | |
| | BINDING STRUCTURE | CD19 Positive B-cell NHL or B- | |
| | | precursor ALL | |
| | | 2023 Topp M, Dlugosz-D . . . Safety of | |
| | | AFM11 in the treatment of patients | |
| | | with B-cell malignancies: findings | |
| | | from two phase 1 studies. | |
| | | 2019 Duell J, Lukic D S . . . Functionally | |
| | | Defective T Cells After | |
| | | Chemotherapy of B-Cell | |
| | | Malignancies Can Be Activated by | |
| | | the Tetravalent Bispecific CD19/CD3 | |
| | | Antibody AFM11. | |
| | | 2015 Reusch U, Duell J . . . A | |
| | | tetravalent bispecific TandAb | |
| | | (CD19/CD3), AFM11, efficiently | |
| | | recruits T cells for the potent lysis | |
| | | of CD19(+) tumor cells. | |
| | | 2002 Kipriyanov S M, Co . . . | |
| | | Synergistic antitumor effect of | |
| | | bispecific CD19 × CD3 and CD19 × | |
| | | CD16 diabodies in a preclinical | |
| | | model of non-Hodgkin's lymphoma. | |
| | | ASCO 2012 High affinity CD3 | |
| | | RECRUIT TandAb for T cell- | |
| | | mediated lysis of CD19+ tumor B | |
| | | cells Eugene Zhukovsky, . . . | |
| | | ASCO 2013 AFM11, a CD19/CD3 | |
| | | bispecific tandab, to facilitate T-cell- | |
| | | mediated killing of CD19+ cells | |
| | | Eugene Zhukovsky, . . . | |
| | | ASH 2013 A CD19/CD3 Bispecific | |
| | | Tandab, AFM11, Recruits T Cells To | |
| | | Potently and Safely Kill CD19+ | |
| | | Tumor Cells Eugene Zhukovsky, . . . | |
| AFM12 | 2006 WO2006125668 | 2004 Reusch U, Le Gall . . . Effect of | Affimed |
| | HOFFMANN, Karin; . . . ANTI- | tetravalent bispecific CD19 × CD3 | |
| | CD16 BINDING MOLECULES | recombinant antibody construct | |
| | 2002 WO2003048209 | and CD28 costimulation on lysis of | |
| | KIPRIYANOV, Serge . . . | malignant B cells from patients with | |
| | BISPECIFIC ANTI-CD19 × ANTI- | chronic lymphocytic leukemia by | |
| | CD16 ANTIBODIES AND USES | autologous T cells. | |
| | THEREOF | 2002 Kipriyanov S M, Co . . . | |
| | | Synergistic antitumor effect of | |
| | | bispecific CD19 × CD3 and CD19 × | |
| | | CD16 diabodies in a preclinical | |
| | | model of non-Hodgkin's lymphoma. | |
| AMG 562 | 2019 US20200362039 Kufer, | 2018 NCT03571828 Phase 1 Study | Amgen |
| | Peter; Rau . . . CROSS-SPECIES- | to Evaluate the Safety, Tolerability, | |
| | SPECIFIC PSCA × CD3, | Pharmacokinetics, and Efficacy of | |
| | CD19 × CD3, C-MET × CD3, | AMG 562 in Subjects With r/r | |
| | ENDOSIALIN × CD3, | Diffuse Large B-cell Lymphoma, | |
| | EPCAM × CD3, IGF-1R × CD3 OR | Mantle Cell Lymphoma, or Follicular | |
| | FAPALPHA × CD3 BISPECIFIC | Lymphoma | |
| | SINGLE CHAIN ANTIBODY | | |
| AMG 729 | | | Amgen |
| Ampsource | 2019 WO2020088605 LI, Qiang, | | Ampsource |
| patent anti-CD19/ | JIA, S . . . HOMODIMER-TYPE | | |
| CD3 | BISPECIFIC ANTIBODY | | |
| | TARGETING CD19 AND CD3, | | |
| | AND PREPARATION METHOD | | |
| | THEREFOR AND APPLICATION | | |
| | THEREOF | | |
| | 2019 US20210371526 Li, | | |
| | Qiang; Ma, Xi . . . HOMODIMERIC | | |
| | BISPECIFIC ANTIBODY, | | |
| | PREPARATION METHOD | | |
| | THEREFOR AND USE THEREOF | | |
| | 2019 US20220002407 Li, | | |
| | Qiang; Jia, S . . . HOMODIMER- | | |
| | TYPE BISPECIFIC ANTIBODY | | |
| | TARGETING CD19 AND CD3, | | |
| | AND PREPARATION METHOD | | |
| | THEREFOR AND APPLICATION | | |
| | THEREOF | | |
| Beijing Luzhu Bio | 2018 US20190284279 KONG, | | Beijing Luzhu Bio |
| patent anti-CD19/ | Jian; YE, Y . . . BISPECIFIC | | |
| CD3 | ANTIBODY BINDING TO | | |
| | HUMAN CD19 AND CD3 | | |
| | 2018 WO2019095641 KONG, | | |
| | Jian, YE, Y . . . BISPECIFIC HUMAN | | |
| | CD19 AND CD3 BINDING | | |
| | ANTIBODY | | |
| BioGraph 55 | 2022 US20220185907 PRESTA, | | BioGraph 55 |
| patent anti-CD19/ | Leonard; . . . ANTI-CD19/ANTI- | | |
| CD38 | CD38 COMMON LIGHT CHAIN | | |
| | BISPECIFIC ANTIBODIES | | |
| | 2022 US20220185906 PRESTA, | | |
| | Leonard; . . . ANTI-CD19/ANTI- | | |
| | CD38 COMMON LIGHT CHAIN | | |
| | BISPECIFIC ANTIBODIES | | |
| | 2022 US20220185908 PRESTA, | | |
| | Leonard; . . . METHODS OF USE | | |
| | OF ANTI-CD19/ANTI-CD38 | | |
| | COMMON LIGHT CHAIN | | |
| | BISPECIFIC ANTIBODIES | | |
| | 2022 US20220363774 PRESTA, | | |
| | Leonard; . . . ANTI-CD19/ANTI- | | |
| | CD38 COMMON LIGHT CHAIN | | |
| | BISPECIFIC ANTIBODIES | | |
| | 2021 U.S. Pat. No. 11,299,551 Presta, | | |
| | Leonard ( . . . Composite binding | | |
| | molecules targeting | | |
| | immunosuppressive B cells | | |
| | 2021 WO2021173844 PRESTA, | | |
| | Leonard, . . . C19 C38 BISPECIFIC | | |
| | ANTIBODIES | | |
| blinatumomab | 2021 US20220088196 Bauerle, | 2022 NCT05327894 Phase 3 | Amgen |
| | Patrick; . . . Means and Methods | Interfant-21 Treatment Protocol for | Medimmune |
| | for Treating Tumorous Diseases | Infants Under 1 Year With KMT2A- | Micromet |
| | 2020 WO2021055528 | rearranged ALL or Mixed Phenotype | |
| | MULLIGHAN, Charle . . . | Acute Leukemia | |
| | METHODS AND KIT FOR | 2021 NCT04827745 Phase 2 | |
| | ANALYZING RESPONSIVENESS | Blinatumomab for Treatment of R/R | |
| | OF PATIENTS TO CD19 | or MRD-positive CD19-Positive | |
| | IMMUNOTHERAPY | MPAL | |
| | 2020 US20210040234 June, | 2021 NCT04722848 Phase 3 | |
| | Carl H.; Zh . . . Enhancing Activity | Sequential Treatment With | |
| | of CAR T Cells by Co- | Ponatinib and Blinatumomab vs | |
| | Introducing a Bispecific | Chemotherapy and Imatinib in | |
| | Antibody | Newly Diagnosed Adult Ph+ ALL | |
| | 2020 WO2020221792 | 2021 NCT04746209 Phase 2 | |
| | ZUGMAIER, Gerhard . . . MEANS | Blinatumomab After TCR Alpha | |
| | AND METHODS OF TREATING | Beta/CD19 Depleted HCT | |
| | BURKITT LYMPHOMA OR | 2021 NCT04521231 Phase 1 A Study | |
| | LEUKEMIA | of Subcutaneous Blinatumomab | |
| | 2020 US20220204620 | Administration in Acute | |
| | Zugmaier, Gerhard . . . MEANS | Lymphoblastic Leukemia (ALL) | |
| | AND METHODS FOR TREATING | Patients | |
| | BURKITT LYMPHOMA OR | 2020 NCT04546399 Phase 2 A Study | |
| | LEUKEMIA | to Compare Blinatumomab Alone to | |
| | 2019 US20200362039 Kufer, | Blinatumomab With Nivolumab in | |
| | Peter; Rau . . . CROSS-SPECIES- | Patients Diagnosed With First | |
| | SPECIFIC PSCA × CD3, | Relapse B-Cell Acute Lymphoblastic | |
| | CD19 × CD3, C-MET × CD3, | Leukemia (B-ALL) | |
| | ENDOSIALIN × CD3, | 2020 NCT04530565 Phase 3 Testing | |
| | EPCAM × CD3, IGF-1R × CD3 OR | the Use of Steroids and Tyrosine | |
| | FAPALPHA × CD3 BISPECIFIC | Kinase Inhibitors With | |
| | SINGLE CHAIN ANTIBODY | Blinatumomab or Chemotherapy | |
| | 2019 US20200071405 Xiao, | for Newly Diagnosed BCR-ABL- | |
| | Shouhua; Pa . . . ANTIBODY | Positive Acute Lymphoblastic | |
| | CONSTRUCTS FOR CDH19 AND | Leukemia in Adults | |
| | CD3 | 2020 NCT04524455 Phase 1 A Study | |
| | 2018 US20190300609 | of Blinatumomab in Combination | |
| | Zugmaier, Gerhard . . . | With AMG 404 for the Treatment of | |
| | TREATMENT OF ACUTE | Adults With Acute Lymphoblastic | |
| | LYMPHOBLASTIC LEUKEMIA | Leukemia (ALL) | |
| | 2018 US20190293635 | 2020 NCT04448834 Phase 2 | |
| | Nagorsen, Dirk; DOSAGE | Blincyto Amgen Acrotech | |
| | REGIMEN FOR ADMINISTERING | BioPharma PH2 Blincyto Marqibo | |
| | A CD19 × CD3 BISPECIFIC | R/R Philadelphi CD19+ ALL | |
| | ANTIBODY TO PATIENTS AT | 2020 NCT04334993 Phase 2 | |
| | RISK FOR POTENTIAL ADVERSE | Pediatric-type Therapy With Pre- | |
| | EFFECTS | transplant Blinatumomab for HR | |
| | 2018 WO2019075366 | Patients - Phase II Study | |
| | ZIMMERMAN, Zachar . . . | 2020 NCT04329325 Phase 2 | |
| | COMPOSITIONS AND | Blinatumomab and Tyrosine Kinase | |
| | METHODS FOR TREATING | Inhibitor Therapy in People With | |
| | DIFFUSE LARGE B CELL | Philadelphia Chromosome-Positive | |
| | LYMPHOMA | Acute Lymphoblastic Leukemia | |
| | 2018 US20190151448 Abel, | 2019 NCT04044560 Phase 2 | |
| | Jeff; Cui, . . . PHARMACEUTICAL | Blinatumomab for MRD in Pre-B ALL | |
| | COMPOSITION COMPRISING | Patients Following Stem Cell | |
| | BISPECIFIC ANTIBODY | Transplant | |
| | CONSTRUCTS FOR IMPROVED | 2019 NCT03914625 Phase 3 | |
| | STORAGE AND | Blinatumomab in Combination With | |
| | ADMINISTRATION | Chemotherapy in Treating Patients | |
| | 2017 US20170209571 | With or Without Down Syndrome | |
| | Kanapuram, Sekhar . . . | and Newly Diagnosed, Standard | |
| | PHARMACEUTICAL | Risk B-Lymphoblastic Leukemia or | |
| | COMPOSITION COMPRISING | Localized B-Lymphoblastic | |
| | BISPECIFIC ANTIBODY | Lymphoma | |
| | CONSTRUCTS | 2019 NCT03982992 Phase 2 | |
| | 2017 U.S. Pat. No. 11,419,933 Kanapuram, | Allogeneic Donor Lymphocyte | |
| | Sekhar . . . Pharmaceutical | Infusions Combined With | |
| | composition comprising | Blinatumomab | |
| | bispecific antibody constructs | 2019 NCT03931642 Phase 2 | |
| | 2015 US20160208001 | BLINAtumomab After R-CHOP | |
| | Zugmaier, Gerhard . . . MEANS | Debulking Therapy for Patients | |
| | AND METHODS FOR TREATING | With Richter Transformation | |
| | DLBCL | 2019 NCT03571321 Phase 1 | |
| | 2015 U.S. Pat. No. 10,696,744 Zugmaier, | Ruxolitinib and Chemotherapy in | |
| | Gerhard . . . Means and methods | Adolescents and Young Adults With | |
| | for treating DLBCL | Ph-like Acute Lymphoblastic | |
| | 2015 U.S. Pat. No. 9,765,157 Xiao, | Leukemia | |
| | Shouhua; Pa . . . Antibody | 2019 NCT03628053 Phase 3 | |
| | constructs for CDH19 and CD3 | Tisagenlecleucel vs Blinatumomab | |
| | 2014 US20150071928 | or Inotuzumab for Patients With | |
| | Nagorsen, Dirk; K . . . DOSAGE | Relapsed/Refractory B-cell | |
| | REGIMEN FOR ADMINISTERING | Precursor Acute Lymphoblastic | |
| | A CD19 × CD3 BISPECIFIC | Leukemia | |
| | ANTIBODY | 2019 NCT03849651 Phase 2 TCRαβ- | |
| | 2014 U.S. Pat. No. 10,662,243 Nagorsen, | DEPLETED PROGENITOR CELL | |
| | Dirk (M . . . Dosage regimen for | GRAFT WITH ADDITIONAL MEMORY | |
| | administering a CD19 × CD3 | T-CELL DLI, PLUS SELECTED USE OF | |
| | bispecific antibody | BLINATUMOMAB, IN NAÏVE T-CELL | |
| | 2014 U.S. Pat. No. 9,486,475 Kufer, Peter; | DEPLETED HAPLOIDENTICAL | |
| | Nag . . . PPS for the prevention of | DONOR HEMATOPOIEITC CELL | |
| | potential adverse effects | TRANSPLANTATION FOR | |
| | caused by CD3 specific binding | HEMATOLOGIC MALIGNANCIES | |
| | domains | 2018 NCT03751709 Phase 1 | |
| | 2014 U.S. Pat. No. 9,688,760 Kufer, Peter; | Blinatumomab Plus HLA- | |
| | Nag . . . Anti-leukocyte adhesion | Mismatched Cellular Therapy for | |
| | for the mitigation of potential | Relapsed/Refractory CD19+ ALL | |
| | adverse events caused by CD3- | 2018 NCT03823365 Phase 1 | |
| | specific binding domains | Blinatumomab Expanded T-cells | |
| | 2012 WO2012146394 | (BET) in Indolent Non-Hodgkin | |
| | NAGORSEN, Dirk DOSAGE | Lymphomas/Chronic Lymphocytic | |
| | REGIMEN FOR ADMINISTERING | Leukemia | |
| | A CD19 × CD3 BISPECIFIC | 2018 NCT03739814 Phase 2 | |
| | ANTIBODY TO PATIENTS AT | Inotuzumab Ozogamicin and | |
| | RISK FOR POTENTIAL ADVERSE | Blinatumomab in Treating Patients | |
| | EFFECTS | With Newly Diagnosed, Recurrent, | |
| | 2012 US20140199307 | or Refractory CD22-Positive B- | |
| | Nagorsen, Dirk; DOSAGE | Lineage Acute Lymphoblastic | |
| | REGIMEN FOR ADMINISTERING | Leukemia | |
| | A CD19 × CD3 BISPECIFIC | 2018 NCT03709719 Phase 2 A | |
| | ANTIBODY TO PATIENTS AT | Phase II Study on Blinatumomab in | |
| | RISK FOR POTENTIAL ADVERSE | High-risk B-cell Precursor Acute | |
| | EFFECTS | Lymphoblastic Leukemia | |
| | 2012 U.S. Pat. No. 10,191,034 Nagorsen, | 2018 NCT03512405 Phase 1/Phase | |
| | Dirk (M . . . Dosage regimen for | 2 Pembrolizumab and | |
| | administrating a CD19 × CD3 | Blinatumomab in Treating | |
| | bispecific antibody to patients | Participants With Recurrent or | |
| | at risk for potential adverse | Refractory Acute Lymphoblastic | |
| | effects | Leukemia | |
| | 2011 WO2012055961 | 2018 NCT03605589 Phase 1 | |
| | ZUGMAIER, Gerhard . . . MEANS | Pembro + Blina Combination in | |
| | AND METHODS FOR TREATING | Pediatric and Young Adult Patients | |
| | DLBCL | With Relapsed/Refractory Acute | |
| | 2011 US20130287778 | Leukemia or Lymphoma | |
| | Zugmaier, Gerhard . . . MEANS | 2018 NCT03643276 Phase 3 | |
| | AND METHODS FOR TREATING | Treatment Protocol for Children | |
| | DLBCL | and Adolescents With Acute | |
| | 2011 U.S. Pat. No. 9,192,665 Zugmaier, | Lymphoblastic Leukemia - AIEOP- | |
| | Gerhard . . . Means and methods | BFM ALL 2017 | |
| | for treating DLBCL | 2018 NCT03523429 Phase 2 | |
| | 2011 US20130095103 | PETHEMA-BLIN-01/PET069014 | |
| | Baeuerle, Patrick . . . MEANS | (BLIN-01) | |
| | AND METHODS FOR THE | 2018 NCT03541083 Phase 2 | |
| | TREAMENT OF TUMOROUS | Blinatumomab Added to Prephase | |
| | DISEASES | and Consolidation Therapy in | |
| | 2011 U.S. Pat. No. 11,154,617 Baeuerle, | Precursor B-acute Lymphoblastic | |
| | Patrick . . . Means and methods | Leukemia in Adults. | |
| | for the treament of B cell non- | 2018 NCT03480438 Phase 2 | |
| | hodgkin lymphoma and B cell | Treatment of Older Patients With B- | |
| | leukemia | precursor ALL With Sequential Dose | |
| | 2010 US20120328618 | Reduced Chemotherapy and | |
| | Nagorsen, Dirk; K . . . DOSAGE | Blinatumomab | |
| | REGIMEN FOR ADMINISTERING | 2018 NCT03518112 Phase 2 Low- | |
| | A CD19 × CD3 BISPECIFIC | Intensity Chemotherapy and | |
| | ANTIBODY | Blinatumomab in Patients With | |
| | 2010 U.S. Pat. No. 8,840,888 Nagorsen, | Philadelphia Chromosome Negative | |
| | Dirk; K . . . Dosage regimen for | Relapsed/Refractory Acute | |
| | administering a CD19 × CD3 | Lymphoblastic Leukemia (ALL) | |
| | bispecific antibody | 2018 NCT03298412 Phase 2 Effect | |
| | 2010 WO2011051307 | of Blinatumomab on MRD in DLBCL | |
| | NAGORSEN, Dirk, K . . . DOSAGE | Subjects Post aHSCT | |
| | REGIMEN FOR ADMINISTERING | 2018 NCT03318770 N/A Post- | |
| | A CD19 × CD3 BISPECIFIC | Frontline Sequential Treatment of | |
| | ANTIBODY | Adult Philadelphia Chromosome- | |
| | 2009 WO2010052013 | Positive Acute Lymphoblastic | |
| | ZUGMAIER, Gerhard; | Leukemia | |
| | Treatment of pediatric acute | 2018 NCT03340766 Phase 1 Open | |
| | lymphoblastic leukemia | Label Study Investigating the Safety | |
| | 2009 WO2010052014 | and Efficacy of Blinatumomab in | |
| | ZUGMAIER, Gerhard . . . | Combination With Pembrolizumab | |
| | Treatment of acute | (KEYNOTE-348) | |
| | lymphoblastic leukemia | 2018 NCT03367299 Phase 2 | |
| | 2009 US20110262440 | Sequential Chemotherapy and | |
| | Zugmaier, Gerhard | Blinatumomab to Improve Minimal | |
| | TREATMENT OF PEDIATRIC | Residual Disease Response and | |
| | ACUTE LYMPHOBLASTIC | Survival in Acute Lymphoblastic | |
| | LEUKEMIA | Leukemia | |
| | 2009 US20130323247 | 2017 NCT03263572 Phase 2 | |
| | Zugmaier, Gerhard . . . | Blinatumomab and Ponatinib in | |
| | TREATMENT OF ACUTE | Patients With Philadelphia | |
| | LYMPHOBLASTIC LEUKEMIA | Chromosome (Ph)-Positive and/or | |
| | 2008 WO2009070642 | BCR-ABL Positive Acute | |
| | SHARMA, Monika, S . . . PROTEIN | Lymphoblastic Leukemia (ALL) | |
| | FORMULATION | 2017 NCT03476239 Phase 3 Efficacy | |
| | 2008 U.S. Pat. No. 9,308,257 Sharma; Sr., | and Safety of the BiTE Antibody | |
| | Moni . . . Protein formulation | Blinatumomab in Chinese Adult | |
| | 2008 US20100303827 Sharma; | Subjects With Relapsed/Refractory | |
| | SR., Moni . . . Protein | B-precursor Acute Lymphoblastic | |
| | Formulation | Leukemia (ALL) | |
| | 2008 US20100150918 Kufer, | 2017 NCT03160079 Phase 1/Phase | |
| | Peter; Rau . . . CROSS-SPECIES- | 2 Blinatumomab and | |
| | SPECIFIC BINDING DOMAIN | Pembrolizumab for Adults With | |
| | 2006 US20090291072 | Relapsed/Refractory B-cell Acute | |
| | Baeuerle, Patrick . . . MEANS | Lymphoblastic Leukemia With High | |
| | AND METHODS FOR THE | Marrow Lymphoblasts | |
| | TREATMENT OF TUMOROUS | 2017 NCT02997761 Phase 2 | |
| | DISEASES | Ibrutinib and Blinatumomab in | |
| | 2006 WO2007068354 | Treating Patients With Relapsed or | |
| | BAEUERLE, Patrick . . . MEANS | Refractory B Acute Lymphoblastic | |
| | AND METHODS FOR THE | Leukemia | |
| | TREATMENT OF TUMOROUS | 2017 NCT03072771 Phase 1 | |
| | DISEASES | Blinatumomab Consolidation Post | |
| | 2006 U.S. Pat. No. 8,007,796 Baeuerle, | Autologous Stem Cell | |
| | Patrick . . . Means and methods | Transplantation in Patients With | |
| | for the treatment of tumorous | Diffuse Large B-Cell Lymphoma | |
| | diseases | (DLBCL) | |
| | 2006 US20060193852 Dorken, | 2017 NCT03121534 Phase 2 Study | |
| | Bernd; Ri . . . Novel CD19 × CD3 | of Blinatumomab in Richter | |
| | specific polypeptides and uses | Transformation | |
| | thereof | 2017 NCT02879695 Phase 1 | |
| | 2006 U.S. Pat. No. 7,575,923 Dorken, | Blinatumomab and Nivolumab With | |
| | Bernd; Ri . . . CD19 × CD3 specific | or Without Ipilimumab in Treating | |
| | polypeptides and uses thereof | Patients With Poor-Risk Relapsed or | |
| | 2005 U.S. Pat. No. 8,101,722 Kufer, Peter; | Refractory CD19+ Precursor B- | |
| | Len . . . Less immunogenic | Lymphoblastic Leukemia | |
| | binding molecules | 2017 NCT03114865 Early Phase 1 A | |
| | 2004 US20070249529 | Study of Blinatumomab in Patients | |
| | Hofmeister, Rober . . . | With Pre B-cell ALL and B-cell NHL | |
| | Compositions Comprising | as Post-allo-HSCT Remission | |
| | Polypeptides | Maintenance | |
| | 2004 WO2005052004 | 2017 NCT03173430 Early Phase 1 | |
| | HOFMEISTER, Rober . . . | Pilot Study of Blinatumomab in | |
| | Compositions comprising | Combination With Salvage | |
| | polypeptides | Autologous Stem Cell | |
| | 2004 U.S. Pat. No. 10,000,574 Hofmeister, | Transplantation for Patients With | |
| | Rober . . . Compositions | Refractory Multiple Myeloma | |
| | comprising polypeptides | 2017 NCT03023878 Phase 2 Safety | |
| | 2004 US20070123479 Kufer, | and Efficacy of Blinatuomab in | |
| | Peter; Lut . . . Pharmaceutical | Subjects With Newly Diagnosed | |
| | compositions comprising | High-risk Diffuse Large B-Cell | |
| | bispecific anti-cd3, anti-cd19 | Lymphoma | |
| | antibody constructs for the | 2016 NCT02877303 Phase 2 Hyper- | |
| | treatment of b-cell related | CVAD Regimen in Sequential | |
| | disorders | Combination With Blinatumomab | |
| | 2004 U.S. Pat. No. 7,635,472 Kufer, Peter; | as Frontline Therapy for Adults | |
| | Lut . . . Pharmaceutical | With B-Cell Lineage Acute | |
| | compositions comprising | Lymphocytic Leukemia | |
| | bispecific anti-cd3, anti-cd19 | 2016 NCT02807883 Phase 2 | |
| | antibody constructs for the | Blinatumomab Maintenance | |
| | treatment of b-cell related | Following Allogeneic Hematopoietic | |
| | disorders | Cell Transplantation for Patients | |
| | 2004 WO2004106380 KUFER, | With Acute Lymphoblastic | |
| | Peter; RAU . . . Human-anti- | Leukemia | |
| | human cd3 binding molecules | 2016 NCT02811679 Phase 2 A Study | |
| | 2004 WO2004106381 KUFER, | Of Blinatumomab For The | |
| | Peter; LUT . . . Pharmaceutical | Treatment Of Relapsed Or | |
| | compositions comprising | Refractory Indolent Non-Hodgkin | |
| | bispecific anti-cd3, anti-cd19 | Lymphoma | |
| | antibody constructs for the | 2016 NCT02568553 Phase 1 | |
| | treatment of b-cell related | Lenalidomide and Blinatumomab in | |
| | disorders | Treating Patients With Relapsed | |
| | 1999 U.S. Pat. No. 7,112,324 Dorken, | Non-Hodgkin Lymphoma | |
| | Bernd; Ri . . . CD 19.times.CD3 | 2016 NCT02790515 Phase 2 | |
| | specific polypeptides and uses | Provision of TCRγδ T Cells and | |
| | thereof | Memory T Cells Plus Selected Use of | |
| | 1999 WO1999054440 DÖRKEN, | Blinatumomab in Naïve T-cell | |
| | Bernd; RI . . . Cd19 × cd3 specific | Depleted Haploidentical Donor | |
| | polypeptides and uses thereof | Hematopoietic Cell Transplantation | |
| | | for Hematologic Malignancies | |
| | | Relapsed or Refractory Despite | |
| | | Prior Transplantation | |
| | | 2015 NCT02458014 Phase 2 Study | |
| | | of Blinatumomab in Patients With | |
| | | B-cell Lineage Acute Lymphocytic | |
| | | Leukemia With Positive Minimal | |
| | | Residual Disease | |
| | | 2015 NCT02393859 Phase 3 Phase 3 | |
| | | Trial of Blinatumomab Versus | |
| | | Standard Chemotherapy in | |
| | | Pediatric Subjects With HR First | |
| | | Relapse B-precursor ALL | |
| | | 2015 NCT02412306 Phase 1/Phase | |
| | | 2 Study of Blinatumomab in | |
| | | Japanese Adults With | |
| | | Relapsed/Refractory B-precursor | |
| | | Acute Lymphoblastic Leukemia | |
| | | 2014 NCT02143414 Phase 2 | |
| | | Blinatumomab and Combination | |
| | | Chemotherapy or Dasatinib, | |
| | | Prednisone, and Blinatumomab in | |
| | | Treating Older Patients With Newly | |
| | | Diagnosed Acute Lymphoblastic | |
| | | Leukemia | |
| | | 2014 NCT02101853 Phase 3 | |
| | | Blinatumomab in Treating Younger | |
| | | Patients With Relapsed B-Cell Acute | |
| | | Lymphoblastic Leukemia | |
| | | 2014 NCT02013167 Phase 3 Ph 3 | |
| | | Trial of Blinatumomab vs | |
| | | Investigator's Choice of | |
| | | Chemotherapy in Patients With | |
| | | Relapsed or Refractory ALL | |
| | | 2013 NCT02003222 Phase 3 | |
| | | Combination Chemotherapy With | |
| | | or Without Blinatumomab in | |
| | | Treating Patients With Newly | |
| | | Diagnosed BCR-ABL-Negative B | |
| | | Lineage Acute Lymphoblastic | |
| | | Leukemia | |
| | | 2012 NCT01741792 Phase 2 Clinical | |
| | | Study With Blinatumomab in | |
| | | Patients With Relapsed/Refractory | |
| | | Diffuse Large B-cell Lymphoma | |
| | | (DLBCL) | |
| | | 2012 NCT01471782 Phase 1/Phase | |
| | | 2 Clinical Study With Blinatumomab | |
| | | in Pediatric and Adolescent Patients | |
| | | With Relapsed/Refractory B- | |
| | | precursor Acute Lymphoblastic | |
| | | Leukemia | |
| | | 2011 NCT01466179 Phase 2 Clinical | |
| | | Study With Blinatumomab in | |
| | | Patients With Relapsed/Refractory | |
| | | B-precursor Acute Lymphoblastic | |
| | | Leukemia (ALL) | |
| | | 2010 NCT01207388 Phase 2 | |
| | | Confirmatory Phase II Study of | |
| | | Blinatumomab (MT103) in Patients | |
| | | With Minimal Residual Disease of B- | |
| | | precursor ALL | |
| | | 2010 NCT01209286 Phase 2 Study | |
| | | of the BiTE ® Blinatumomab | |
| | | (MT103) in Adult Patients With | |
| | | Relapsed/Refractory B-Precursor | |
| | | Acute Lymphoblastic Leukemia | |
| | | (ALL) | |
| | | 2007 NCT00560794 Phase 2 Phase II | |
| | | Study of the BiTEÂ ® Blinatumomab | |
| | | (MT103) in Patients With Minimal | |
| | | Residual Disease of B-precursor | |
| | | Acute ALL | |
| | | 2004 NCT00274742 Phase 1 Safety | |
| | | Study of the Bispecific T-cell | |
| | | Engager Blinatumomab (MT103) in | |
| | | Patients With Relapsed NHL | |
| | | NCT02187354 N/A Expanded Access | |
| | | Protocol of Blinatumomab in | |
| | | Pediatric and Adolescent Subjects | |
| | | With Relapsed and/or Refractory B- | |
| | | precursor Acute Lymphoblastic | |
| | | Leukemia (ALL) | |
| | | 2023 Rao C K, Kamoroff . . . Super- | |
| | | refractory status epilepticus during | |
| | | blinatumomab initiation for B-cell | |
| | | acute lymphoblastic leukemia. | |
| | | 2022 Zhou H, Yin Q, Ji . . . Efficacy and | |
| | | safety of blinatumomab in Chinese | |
| | | adults with Ph-negative | |
| | | relapsed/refractory B-cell precursor | |
| | | acute lymphoblastic leukemia: A | |
| | | multicenter open-label single-arm | |
| | | China registrational study. | |
| | | 2022 Advani A S, Mosele . . . | |
| | | Dasatinib/Prednisone Induction | |
| | | Followed by | |
| | | Blinatumomab/Dasatinib in Ph+ | |
| | | Acute Lymphoblastic Leukemia. | |
| | | 2022 León A G, Mejía-Ar . . . | |
| | | Blinatumomab plus hyper-CVAD: | |
| | | the prelude to a new era in acute | |
| | | lymphocytic leukaemia. | |
| | | 2022 Tomb P E, Shikdar . . . ALL-180 A | |
| | | Case of Refractory Philadelphia-Like | |
| | | Acute Lymphoblastic Leukemia | |
| | | Treated With | |
| | | Ruxolitinib/Blinatumomab and | |
| | | Ruxolitinib/Inotuzumab Ozogamicin | |
| | | Prior to Allogeneic Marrow | |
| | | Transplant. | |
| | | 2022 Haddad F G, Kantar . . . ALL-424 | |
| | | Updated Results from the Phase II | |
| | | Study of Blinatumomab in | |
| | | Combination With Ponatinib in | |
| | | Philadelphia Chromosome-Positive | |
| | | Acute Lymphoblastic Leukemia. | |
| | | 2022 Thompson P A, Jian . . . A phase | |
| | | two study of high dose | |
| | | blinatumomab in Richter's | |
| | | syndrome. | |
| | | 2022 Mocquot P, Mossaz . . . The | |
| | | pharmacology of blinatumomab: | |
| | | state of the art on | |
| | | pharmacodynamics, | |
| | | pharmacokinetics, adverse drug | |
| | | reactions and evaluation in clinical | |
| | | trials. | |
| | | 2022 Edahiro T, Fukush . . . | |
| | | Progressive multifocal | |
| | | leukoencephalopathy in relapsed | |
| | | Ph+ acute lymphoblastic leukemia | |
| | | after cord blood transplantation | |
| | | and blinatumomab treatment: A | |
| | | case report and literature review. | |
| | | 2022 Jabbour E J, Short . . . | |
| | | Blinatumomab is associated with | |
| | | favorable outcomes in patients with | |
| | | B-cell lineage acute lymphoblastic | |
| | | leukemia and positive measurable | |
| | | residual disease at a threshold of | |
| | | 10-4 and higher. | |
| | | 2022 Yeoh D K, Blyth C C . . . | |
| | | Blinatumomab as bridging therapy | |
| | | in paediatric B-cell acute | |
| | | lymphoblastic leukaemia | |
| | | complicated by invasive fungal | |
| | | disease. | |
| | | 2022 Duffy C, Santana . . . Evaluating | |
| | | blinatumomab implementation in | |
| | | low- and middle-income countries: | |
| | | a study protocol. | |
| | | 2022 Janardan S, Horwi . . . | |
| | | Blinatumomab induces complete | |
| | | response in refractory PTLD after | |
| | | hematopoietic cell transplantation. | |
| | | 2022 Kauer J, Märklin . . . BCR::ABL1 | |
| | | tyrosine kinase inhibitors hamper | |
| | | the therapeutic efficacy of | |
| | | blinatumomab in vitro. | |
| | | 2022 Heraudet L, Galti . . . VANDA | |
| | | regimen followed by blinatumomab | |
| | | leads to favourable outcome in | |
| | | patients with Philadelphia | |
| | | chromosome-negative B-precursor | |
| | | acute lymphoblastic leukaemia in | |
| | | first relapse. | |
| | | 2022 Locatelli F, Ecke . . . | |
| | | Blinatumomab overcomes poor | |
| | | prognostic impact of measurable | |
| | | residual disease in pediatric high- | |
| | | risk first relapse B-cell precursor | |
| | | acute lymphoblastic leukemia. | |
| | | 2022 Short N J, Macaron . . . Dismal | |
| | | outcomes of patients with | |
| | | relapsed/refractory Philadelphia | |
| | | chromosome-negative B-cell acute | |
| | | lymphoblastic leukemia after failure | |
| | | of both inotuzumab ozogamicin and | |
| | | blinatumomab. | |
| | | 2022 Tashiro Y, Kanda . . . Feasibility | |
| | | of ovarian stimulation for fertility | |
| | | preservation during and after | |
| | | blinatumomab treatment for Ph- | |
| | | negative B-cell acute lymphoblastic | |
| | | leukemia. | |
| | | 2022 Advani A S, Mosele . . . SWOG | |
| | | 1318: A Phase II Trial of | |
| | | Blinatumomab Followed by POMP | |
| | | Maintenance in Older Patients With | |
| | | Newly Diagnosed Philadelphia | |
| | | Chromosome-Negative B-Cell Acute | |
| | | Lymphoblastic Leukemia. | |
| | | 2022 Ohana Z, Serraes . . . | |
| | | Cytogenetic guided therapy using | |
| | | blinatumomab and inotuzumab | |
| | | ozogamicin in a patient with | |
| | | relapse/refractory acute | |
| | | lymphoblastic leukemia. | |
| | | 2022 Wudhikarn K, King . . . | |
| | | Outcomes of Relapsed B-Cell Acute | |
| | | Lymphoblastic Leukemia After | |
| | | Sequential Treatment with | |
| | | Blinatumomab and Inotuzumab. | |
| | | 2022 Sharplin K M, Marks D The | |
| | | treatment landscape for Relapsed | |
| | | Refractory B Acute Lymphoblastic | |
| | | Leukaemia (ALL). | |
| | | 2021 Wang S, Peng L, X . . . Preclinical | |
| | | characterization and comparison | |
| | | between CD3/CD19 bispecific and | |
| | | novel CD3/CD19/CD20 trispecific | |
| | | antibodies against B-cell acute | |
| | | lymphoblastic leukemia: targeted | |
| | | immunotherapy for acute | |
| | | lymphoblastic leukemia. | |
| | | 2021 Jabbour E, Patel . . . Impact of | |
| | | Philadelphia chromosome-like | |
| | | alterations on efficacy and safety of | |
| | | blinatumomab in adults with | |
| | | relapsed/refractory acute | |
| | | lymphoblastic leukemia: A post hoc | |
| | | analysis from the phase 3 TOWER | |
| | | study. | |
| | | 2021 Mikhailova E, Glu . . . | |
| | | Immunophenotypic changes of | |
| | | leukemic blasts in children with | |
| | | relapsed/refractory B-cell precursor | |
| | | acute lymphoblastic leukemia, who | |
| | | have been treated with | |
| | | Blinatumomab. | |
| | | 2021 Kobayashi Y, Oh I . . . Efficacy | |
| | | and safety of blinatumomab: Post | |
| | | hoc pooled analysis in Asian adults | |
| | | with relapsed/refractory B-cell | |
| | | precursor acute lymphoblastic | |
| | | leukemia. | |
| | | 2021 Chen Y H, Wang Y, . . . The | |
| | | potential of adoptive transfer of | |
| | | γ9δ2 T cells to enhance | |
| | | blinatumomab's antitumor activity | |
| | | against B-cell malignancy. | |
| | | 2021 Ramdeny S, Chaudh . . . Activity | |
| | | of blinatumomab in lymphoblastic | |
| | | leukemia with impaired T-cell | |
| | | immunity due to congenital | |
| | | immunodeficiency. | |
| | | 2021 Yuda J, Yamauchi . . . Molecular | |
| | | remission after combination | |
| | | therapy with blinatumomab and | |
| | | ponatinib with relapsed/refractory | |
| | | Philadelphia chromosome-positive | |
| | | acute lymphocytic leukemia: two | |
| | | case reports. | |
| | | 2021 Jeyakumar N, Aldo . . . Cytokine | |
| | | Gene Polymorphisms are | |
| | | Associated with Response to | |
| | | Blinatumomab in B Cell Acute | |
| | | Lymphoblastic Leukemia. | |
| | | 2021 Brown P A, Ji L, X . . . Effect of | |
| | | Postreinduction Therapy | |
| | | Consolidation With Blinatumomab | |
| | | vs Chemotherapy on Disease-Free | |
| | | Survival in Children, Adolescents, | |
| | | and Young Adults With First Relapse | |
| | | of B-Cell Acute Lymphoblastic | |
| | | Leukemia: A Randomized Clinical | |
| | | Trial. | |
| | | 2021 Kamachi K, Ureshi . . . Dasatinib- | |
| | | Blinatumomab for Ph-Positive ALL. | |
| | | 2021 Foà R, Chiaretti S Dasatinib- | |
| | | Blinatumomab for Ph-Positive ALL. | |
| | | Reply. | |
| | | 2021 Halford Z, Coalte . . . A | |
| | | Systematic Review of | |
| | | Blinatumomab in the Treatment of | |
| | | Acute Lymphoblastic Leukemia: | |
| | | Engaging an Old Problem With New | |
| | | Solutions. | |
| | | 2020 Bernhardt M B, Mil . . . | |
| | | Blinatumomab use in pediatric ALL: | |
| | | Taking a BiTE out of preparation, | |
| | | administration and toxicity | |
| | | challenges. | |
| | | 2020 Contreras C F, Hig . . . Clinical | |
| | | utilization of blinatumomab and | |
| | | inotuzumab immunotherapy in | |
| | | children with relapsed or refractory | |
| | | B-acute lymphoblastic leukemia. | |
| | | 2020 Jasinski S, De Lo . . . | |
| | | Immunotherapy in Pediatric B-Cell | |
| | | Acute Lymphoblastic Leukemia: | |
| | | Advances and Ongoing Challenges. | |
| | | 2020 Guranda C, Anna L . . . Bispecific | |
| | | antibodies in acute lymphoblastic | |
| | | leukemia therapy. | |
| | | 2020 Zhao Y, Aldoss I, . . . Tumor | |
| | | intrinsic and extrinsic determinants | |
| | | of response to blinatumomab in | |
| | | adults with B-ALL. | |
| | | 2020 Hosseini I, Gadka . . . Mitigating | |
| | | the risk of cytokine release | |
| | | syndrome in a Phase I trial of | |
| | | CD20/CD3 bispecific antibody | |
| | | mosunetuzumab in NHL: impact of | |
| | | translational system modeling. | |
| | | 2020 Viardot A, Locate . . . Concepts | |
| | | in immuno-oncology: tackling B cell | |
| | | malignancies with CD19-directed | |
| | | bispecific T cell engager therapies. | |
| | | 2020 Lau K M, Saunders . . . | |
| | | Characterization of relapse patterns | |
| | | in patients with acute lymphoblastic | |
| | | leukemia treated with | |
| | | blinatumomab. | |
| | | 2020 Niyongere S, Sanc . . . Frontline | |
| | | Blinatumomab in Older Adults with | |
| | | Philadelphia Chromosome-Negative | |
| | | B-Cell Acute Lymphoblastic | |
| | | Leukemia. | |
| | | 2020 Fuster J L, Molino . . . | |
| | | Blinatumomab and inotuzumab for | |
| | | B cell precursor acute lymphoblastic | |
| | | leukaemia in children: a | |
| | | retrospective study from the | |
| | | Leukemia Working Group of the | |
| | | Spanish Society of Pediatric | |
| | | Hematology and Oncology (SEHOP). | |
| | | 2020 Apel A, Ofran Y, . . . Safety and | |
| | | efficacy of blinatumomab: a real | |
| | | world data. | |
| | | 2020 Jiang X, Chen X, . . . | |
| | | Development of a minimal | |
| | | physiologically-based | |
| | | pharmacokinetic/pharmacodynamic | |
| | | model to characterize target cell | |
| | | depletion and cytokine release for T | |
| | | cell-redirecting bispecific agents in | |
| | | humans. | |
| | | 2020 Salhotra A, Yang . . . Outcomes | |
| | | of Allogeneic Hematopoietic Cell | |
| | | Transplantation after Salvage- | |
| | | Therapy with Blinatumomab in | |
| | | Patients with Relapsed/Refractory | |
| | | Acute Lymphoblastic Leukemia. | |
| | | 2019 Danylesko I, Chow . . . | |
| | | Treatment with anti CD19 chimeric | |
| | | antigen receptor T cells after | |
| | | antibody-based immunotherapy in | |
| | | adults with acute lymphoblastic | |
| | | leukemia. | |
| | | 2019 Yu J, Wang W, Hua . . . Efficacy | |
| | | and safety of bispecific T-cell | |
| | | engager (BiTE) antibody | |
| | | blinatumomab for the treatment of | |
| | | relapsed/refractory acute | |
| | | lymphoblastic leukemia and non- | |
| | | Hodgkin's lymphoma: a systemic | |
| | | review and meta-analysis. | |
| | | 2019 Ali S, Moreau A, . . . | |
| | | Blinatumomab for Acute | |
| | | Lymphoblastic Leukemia: The First | |
| | | Bispecific T-Cell Engager Antibody | |
| | | to Be Approved by the EMA for | |
| | | Minimal Residual Disease. | |
| | | 2019 Rambaldi A, Riber . . . | |
| | | Blinatumomab compared with | |
| | | standard of care for the treatment | |
| | | of adult patients with | |
| | | relapsed/refractory Philadelphia | |
| | | chromosome-positive B-precursor | |
| | | acute lymphoblastic leukemia. | |
| | | 2019 Bukhari A, Lee S T | |
| | | Blinatumomab: a novel therapy for | |
| | | the treatment of non-Hodgkin's | |
| | | lymphoma. | |
| | | 2019 Dufner V, Sayehli . . . Long-term | |
| | | outcome of patients with | |
| | | relapsed/refractory B-cell non- | |
| | | Hodgkin lymphoma treated with | |
| | | blinatumomab. | |
| | | 2019 Yarali N, Isik M, . . . Bone | |
| | | Marrow Necrosis in a Patient | |
| | | Following Blinatumomab Therapy. | |
| | | 2019 Jabbour E J, Sasak . . . | |
| | | Inotuzumab ozogamicin in | |
| | | combination with low-intensity | |
| | | chemotherapy (mini-HCVD) with or | |
| | | without blinatumomab versus | |
| | | standard intensive chemotherapy | |
| | | (HCVAD) as frontline therapy for | |
| | | older patients with Philadelphia | |
| | | chromosome-negative acute | |
| | | lymphoblastic leukemia: A | |
| | | propensity score analysis. | |
| | | 2019 Liu D, Zhao J, So . . . Clinical trial | |
| | | update on bispecific antibodies, | |
| | | antibody-drug conjugates, and | |
| | | antibody-containing regimens for | |
| | | acute lymphoblastic leukemia. | |
| | | 2019 Xu L, Wang S, Li . . . CD47/SIRPα | |
| | | blocking enhances CD19/CD3- | |
| | | bispecific T cell engager antibody- | |
| | | mediated lysis of B cell | |
| | | malignancies. | |
| | | 2018 Jain T, Litzow M R No free | |
| | | rides: management of toxicities of | |
| | | novel immunotherapies in ALL, | |
| | | including financial. | |
| | | 2018 Burt R, Warcel D, . . . | |
| | | Blinatumomab, a bispecific B-cell | |
| | | and T-cell engaging antibody, in the | |
| | | treatment of B-cell malignancies. | |
| | | 2018 Algeri M, Del Buf . . . Current | |
| | | and future role of bispecific T-cell | |
| | | engagers in pediatric acute | |
| | | lymphoblastic leukemia. | |
| | | 2018 Jung S H, Lee S R, . . . Efficacy | |
| | | and safety of blinatumomab | |
| | | treatment in adult Korean patients | |
| | | with relapsed/refractory acute | |
| | | lymphoblastic leukemia on behalf | |
| | | of the Korean Society of | |
| | | Hematology ALL Working Party. | |
| | | 2018 Jen E Y, Xu Q, Sch . . . FDA | |
| | | Approval: Blinatumomab for | |
| | | patients with B-cell precursor acute | |
| | | lymphoblastic leukemia in | |
| | | morphologic remission with | |
| | | minimal residual disease. | |
| | | 2018 Blinatumomab | |
| | | 2018 Watkins M P, Bartl . . . CD19- | |
| | | targeted immunotherapies for | |
| | | treatment of patients with non- | |
| | | Hodgkin B-cell lymphomas. | |
| | | 2018 Demosthenous C, L . . . | |
| | | Extramedullary relapse and | |
| | | discordant CD19 expression | |
| | | between bone marrow and | |
| | | extramedullary sites in relapsed | |
| | | acute lymphoblastic leukemia after | |
| | | blinatumomab treatment. | |
| | | 2018 Teplyakov A, Obmo . . . Crystal | |
| | | structure of B-cell co-receptor CD19 | |
| | | in complex with antibody B43 | |
| | | reveals an unexpected fold. | |
| | | 2018 Short N J, Kantarj . . . Novel | |
| | | Therapies for Older Adults With | |
| | | Acute Lymphoblastic Leukemia. | |
| | | 2018 Foster J B, Maude S L New | |
| | | developments in immunotherapy | |
| | | for pediatric leukemia. | |
| | | 2017 Jabbour E, Düll J . . . Outcome of | |
| | | patients with relapsed/refractory | |
| | | acute lymphoblastic leukemia after | |
| | | blinatumomab failure: No change in | |
| | | the level of CD19 expression. | |
| | | 2017 Krishnamurthy A, . . . Bispecific | |
| | | antibodies for cancer therapy: A | |
| | | review. | |
| | | 2017 Velasquez M P, Bon . . . | |
| | | Redirecting T cells to hematological | |
| | | malignancies with bispecific | |
| | | antibodies. | |
| | | 2017 Ribera J M Efficacy and safety | |
| | | of bispecific T-cell engager | |
| | | blinatumomab and the potential to | |
| | | improve leukemia-free survival in B- | |
| | | cell acute lymphoblastic leukemia. | |
| | | 2017 Horvat T Z, Seddon . . . The ABCs | |
| | | of Immunotherapy for Adult | |
| | | Patients With B-Cell Acute | |
| | | Lymphoblastic Leukemia. | |
| | | 2017 Wadhwa A, Kutny M . . . | |
| | | Blinatumomab activity in a patient | |
| | | with Down syndrome B-precursor | |
| | | acute lymphoblastic leukemia. | |
| | | 2017 Wilke A C, Gökbuget N Clinical | |
| | | applications and safety evaluation | |
| | | of the new CD19 specific T-cell | |
| | | engager antibody construct | |
| | | blinatumomab. | |
| | | 2017 Assi R, Kantarjia . . . Safety and | |
| | | Efficacy of Blinatumomab in | |
| | | Combination With a Tyrosine Kinase | |
| | | Inhibitor for the Treatment of | |
| | | Relapsed Philadelphia | |
| | | Chromosome-positive Leukemia. | |
| | | 2017 Valecha G K, Ibrah . . . Emerging | |
| | | Role of Immunotherapy in | |
| | | Precursor B-cell Lymphoblastic | |
| | | Leukemia. | |
| | | 2017 Blinatumomab for Acute | |
| | | Lymphoblastic Leukemia. | |
| | | 2017 Blinatumomab for Acute | |
| | | Lymphoblastic Leukemia. | |
| | | 2017 Aldoss I, Song J, . . . Correlates | |
| | | of resistance and relapse during | |
| | | blinatumomab therapy for | |
| | | relapsed/refractory acute | |
| | | lymphoblastic leukemia. | |
| | | 2017 Sanders S, Stewar . . . Targeting | |
| | | non-Hodgkin Lymphoma with | |
| | | Blinatumomab. | |
| | | 2017 Zoghbi A, Zur Sta . . . Lineage | |
| | | switch under blinatumomab | |
| | | treatment of relapsed common | |
| | | acute lymphoblastic leukemia | |
| | | without MLL rearrangement. | |
| | | 2017 Kantarjian H, Ste . . . | |
| | | Blinatumomab versus | |
| | | Chemotherapy for Advanced Acute | |
| | | Lymphoblastic Leukemia. | |
| | | 2017 Yuraszeck T, Kasi . . . Translation | |
| | | and Clinical Development of | |
| | | Bispecific T cell Engaging Antibodies | |
| | | for Cancer Treatment. | |
| | | 2017 Duell J, Dittrich . . . Frequency of | |
| | | regulatory T cells determines the | |
| | | outcome of the T cell engaging | |
| | | antibody blinatumomab in patients | |
| | | with B precursor ALL. | |
| | | 2016 Aldoss I T, Marcuc . . . Treatment | |
| | | of Acute Lymphoblastic Leukemia in | |
| | | Adults: Applying Lessons Learned in | |
| | | Children. | |
| | | 2016 Frey N V, Porter D L Cytokine | |
| | | release syndrome with novel | |
| | | therapeutics for acute | |
| | | lymphoblastic leukemia. | |
| | | 2016 Huguet F, Tavitian S Emerging | |
| | | biological therapies to treat acute | |
| | | lymphoblastic leukemia. | |
| | | 2016 von Stackelberg A . . . Phase | |
| | | I/Phase II Study of Blinatumomab in | |
| | | Pediatric Patients With | |
| | | Relapsed/Refractory Acute | |
| | | Lymphoblastic Leukemia. | |
| | | 2016 Thomas X, Le Jeune C Treating | |
| | | adults with acute lymphocytic | |
| | | leukemia: new pharmacotherapy | |
| | | options. | |
| | | 2016 Zhu M, Wu B, Bran . . . | |
| | | Blinatumomab, a Bispecific T-cell | |
| | | Engager (BiTE( ®)) for CD-19 | |
| | | Targeted Cancer Immunotherapy: | |
| | | Clinical Pharmacology and Its | |
| | | Implications. | |
| | | 2016 Kantarjian H M, St . . . | |
| | | Blinatumomab treatment of older | |
| | | adults with relapsed/refractory B- | |
| | | precursor acute lymphoblastic | |
| | | leukemia: Results from two phase 2 | |
| | | studies. | |
| | | 2016 Goebeler M E, Barg . . . | |
| | | Blinatumomab: a CD19/CD3 | |
| | | bispecific T cell engager (BiTE) with | |
| | | unique anti-tumor efficacy. | |
| | | 2016 Benjamin J E, Stei . . . The role of | |
| | | blinatumomab in patients with | |
| | | relapsed/refractory acute | |
| | | lymphoblastic leukemia. | |
| | | 2016 Rayes A, McMaster . . . Lineage | |
| | | Switch in MLL-Rearranged Infant | |
| | | Leukemia Following CD19-Directed | |
| | | Therapy. | |
| | | 2016 Goebeler M E, Knop . . . | |
| | | Bispecific T-Cell Engager (BiTE) | |
| | | Antibody Construct Blinatumomab | |
| | | for the Treatment of Patients With | |
| | | Relapsed/Refractory Non-Hodgkin | |
| | | Lymphoma: Final Results From a | |
| | | Phase I Study. | |
| | | 2016 Thomas X, Lejeune C | |
| | | Blinatumomab in acute | |
| | | lymphoblastic leukemia. | |
| | | 2016 Marini B L, Wechte . . . | |
| | | Minimizing waste during | |
| | | preparation of blinatumomab | |
| | | infusions. | |
| | | 2016 Viardot A, Goebel . . . Phase 2 | |
| | | study of the bispecific T-cell | |
| | | engager (BiTE) antibody | |
| | | blinatumomab in | |
| | | relapsed/refractory diffuse large B- | |
| | | cell lymphoma. | |
| | | 2016 May M B, Glode A | |
| | | Blinatumomab: A novel, bispecific, | |
| | | T-cell engaging antibody. | |
| | | 2015 Zugmaier G, Gökbu . . . Long- | |
| | | term survival and T-cell kinetics in | |
| | | relapsed/refractory ALL patients | |
| | | who achieved MRD response after | |
| | | blinatumomab treatment. | |
| | | 2015 Köhnke T, Krupka . . . Increase | |
| | | of PD-L1 expressing B-precursor ALL | |
| | | cells in a patient resistant to the | |
| | | CD19/CD3-bispecific T cell engager | |
| | | antibody blinatumomab. | |
| | | 2015 Kaplan J B, Grisch . . . | |
| | | Blinatumomab for the treatment of | |
| | | acute lymphoblastic leukemia. | |
| | | 2015 Wolach O, Stone R M | |
| | | Blinatumomab for the Treatment of | |
| | | Philadelphia Chromosome- | |
| | | Negative, Precursor B-cell Acute | |
| | | Lymphoblastic Leukemia. | |
| | | 2015 Buie L W, Pecoraro . . . | |
| | | Blinatumomab: A First-in-Class | |
| | | Bispecific T-Cell Engager for | |
| | | Precursor B-Cell Acute | |
| | | Lymphoblastic Leukemia. | |
| | | 2015 Stieglmaier J, Be . . . Utilizing | |
| | | the BiTE (bispecific T-cell engager) | |
| | | platform for immunotherapy of | |
| | | cancer. | |
| | | 2015 Sun L L, Ellerman . . . Anti- | |
| | | CD20/CD3 T cell-dependent | |
| | | bispecific antibody for the | |
| | | treatment of B cell malignancies. | |
| | | 2015 Zugmaier G, Kling . . . Clinical | |
| | | overview of anti-CD19 BiTE( ®) and | |
| | | ex vivo data from anti-CD33 BiTE( ®) | |
| | | as examples for retargeting T cells | |
| | | in hematologic malignancies. | |
| | | 2015 Weiland J, Elder . . . CD19: A | |
| | | multifunctional immunological | |
| | | target molecule and its implications | |
| | | for Blineage acute lymphoblastic | |
| | | leukemia. | |
| | | 2015 Oak E, Bartlett N L | |
| | | Blinatumomab for the treatment of | |
| | | B-cell lymphoma. | |
| | | 2015 Sanford M Blinatumomab: | |
| | | First Global Approval. | |
| | | 2014 Topp M S, Gökbuget . . . Phase II | |
| | | Trial of the Anti-CD19 Bispecific T | |
| | | Cell-Engager Blinatumomab Shows | |
| | | Hematologic and Molecular | |
| | | Remissions in Patients With | |
| | | Relapsed or Refractory B-Precursor | |
| | | Acute Lymphoblastic Leukemia. | |
| | | 2014 Zimmerman Z, Mani . . . | |
| | | Unleashing the clinical power of T | |
| | | cells: CD19/CD3 bi-specific T cell | |
| | | engager (BiTE ®) antibody construct | |
| | | blinatumomab as a potential | |
| | | therapy. | |
| | | 2014 Schlegel P, Lang . . . Pediatric | |
| | | posttransplant relapsed/refractory | |
| | | B-precursor acute lymphoblastic | |
| | | leukemia shows durable remission | |
| | | by therapy with the T-cell engaging | |
| | | bispecific antibody blinatumomab. | |
| | | 2012 Nagorsen D, Kufer . . . | |
| | | Blinatumomab: A historical | |
| | | perspective. | |
| | | 2012 Klinger M, Brandl . . . | |
| | | Immunopharmacological response | |
| | | of patients with B-lineage acute | |
| | | lymphoblastic leukemia to | |
| | | continuous infusion of T cell- | |
| | | engaging CD19/CD3-bispecific BiTE | |
| | | antibody blinatumomab. | |
| | | 2011 Brischwein K, Par . . . Strictly | |
| | | target cell-dependent activation of | |
| | | T cells by bispecific single-chain | |
| | | antibody constructs of the BiTE | |
| | | class. | |
| | | 2011 Topp M S, Kufer P, . . . Targeted | |
| | | Therapy With the T-Cell-Engaging | |
| | | Antibody Blinatumomab of | |
| | | Chemotherapy-Refractory Minimal | |
| | | Residual Disease in B-Lineage Acute | |
| | | Lymphoblastic Leukemia Patients | |
| | | Results in High Response Rate and | |
| | | Prolonged Leukemia-Free Survival. | |
| | | 2011 Nagorsen D, Baeue . . . | |
| | | Immunomodulatory therapy of | |
| | | cancer with T cell-engaging BiTE | |
| | | antibody blinatumomab. | |
| | | 2008 d'Argouges S, Wis . . . | |
| | | Combination of rituximab with | |
| | | blinatumomab (MT103/MEDI-538), | |
| | | a T cell-engaging CD19-/CD3- | |
| | | bispecific antibody, for highly | |
| | | efficient lysis of human B | |
| | | lymphoma cells. | |
| | | 2007 Brandl C, Haas C, . . . The effect | |
| | | of dexamethasone on polyclonal T | |
| | | cell activation and redirected target | |
| | | cell lysis as induced by a CD19/CD3- | |
| | | bispecific single-chain antibody | |
| | | construct. | |
| | | ASCO 2013 Pharmacokinetics (PK) | |
| | | of blinatumomab and its clinical | |
| | | implications Benjamin Wu, You . . . | |
| | | ASCO 2013 Blinatumomab exposure | |
| | | and pharmacodynamic response in | |
| | | patients with non-Hodgkin | |
| | | lymphoma (NHL) Youssef Hijazi, M . . . | |
| | | ASCO 2014 Confirmatory open- | |
| | | label, single-arm, multicenter phase | |
| | | 2 study of the BiTE antibody | |
| | | blinatumomab in patients (pts) with | |
| | | relapsed/refractory B-precursor | |
| | | acute lymphoblastic leukemia (r/r | |
| | | ALL) Max S. Topp, Nico . . . | |
| | | ASH 2010 Treatment of Patients | |
| | | with Non-Hodgkin Lymphoma (NHL) | |
| | | with CD19/CD3 Bispecific Antibody | |
| | | Blinatumomab (MT103): Double- | |
| | | Step Dose Increase to Continuous | |
| | | Infusion of 60 μg/m²/d Is Tolerable | |
| | | and Highly Effective Andreas | |
| | | Viardot, . . . | |
| | | ASH 2011 Blinatumomab | |
| | | Monotherapy Shows Efficacy in | |
| | | Patients with Relapsed Diffuse | |
| | | Large B Cell Lymphoma Andreas | |
| | | Viardot, . . . | |
| | | ASH 2012 Anti-CD19 BiTE | |
| | | Blinatumomab Induces High | |
| | | Complete Remission Rate and | |
| | | Prolongs Overall Survival in Adult | |
| | | Patients with Relapsed/Refractory | |
| | | B-Precursor Acute Lymphoblastic | |
| | | Leukemia (ALL) Max S. Topp, Nico . . . | |
| | | ASH 2013 Open-Label Phase 2 Study | |
| | | Of The Bispecific T-Cell Engager | |
| | | (BiTE ®) Blinatumomab In Patients | |
| | | With Relapsed/Refractory Diffuse | |
| | | Large B-Cell Lymphoma Andreas | |
| | | Viardot, . . . | |
| | | ASH 2014 An Evaluation of | |
| | | Molecular Response in a Phase 2 | |
| | | Open-Label, Multicenter | |
| | | Confirmatory Study in Patients (pts) | |
| | | with Relapsed/Refractory B- | |
| | | Precursor Acute Lymphoblastic | |
| | | Leukemia (r/r ALL) Receiving | |
| | | Treatment with the BiTE ® Antibody | |
| | | Construct Blinatumomab Nicola | |
| | | Goekbuget, . . . | |
| | | ASH 2019 A Phase IB Study of | |
| | | Blinatumomab (blina) in Patients | |
| | | with B Cell Acute Lymphoblastic | |
| | | Leukemia (ALL) and B-Cell Non- | |
| | | Hodgkin Lymphoma (NHL) As Post- | |
| | | Allogeneic Blood or Marrow | |
| | | Transplant (allo-BMT) Remission | |
| | | Maintenance Jonathan Webster, . . . | |
| Cellular Biomed. | 2020 WO2021184673 YAO, | | Cellular Biomed. |
| patent anti-CD19/ | Yihong, LI, . . . COMBINED | | |
| CD20 CAR | CHIMERIC ANTIGEN RECEPTOR | | |
| | TARGETING CD19 AND CD20 | | |
| | AND APPLICATION THEREOF | | |
| Chinese | | 2017 Zhang X, Yang Y, . . . | Chinese Acad. |
| Acad. Med. Sci. | | Mesenchymal stromal cells as | Med. Sci. |
| anti-CD19/CD3 | | vehicles of tetravalent bispecific | |
| | | Tandab (CD3/CD19) for the | |
| | | treatment of B cell lymphoma | |
| | | combined with IDO pathway | |
| | | inhibitor D-1-methyl-tryptophan. | |
| | | 2015 Fan D, Li W, Yang . . . | |
| | | Redirection of CD4+ and CD8+ T | |
| | | lymphocytes via an anti-CD3 × anti- | |
| | | CD19 bi-specific antibody combined | |
| | | with cytosine arabinoside and the | |
| | | efficient lysis of patient-derived B- | |
| | | ALL cells. | |
| | | 2012 Wei L, Dongmei F, . . . Disulfide- | |
| | | stabilized diabody | |
| | | antiCD19/antiCD3 exceeds its | |
| | | parental antibody in tumor- | |
| | | targeting activity. | |
| duvortuxizumab | 2018 US20190099489 Mackay, | 2016 NCT02743546 Phase 1 Study | Janssen Biotech |
| | Joanna; Novel Formulations | of the Safety, Pharmacokinetics, | MacroGenics |
| | Which Stabilize Low Dose | Pharmacodynamics, and Efficacy of | |
| | Antibody Compositions | Duvortuxizumab (JNJ-64052781) | |
| | 2016 WO2017096368 BONVINI, | Plus Ibrutinib in Lymphoma | |
| | Ezio, JO . . . BI-SPECIFIC | 2015 NCT02454270 Phase 1 A Dose | |
| | MONOVALENT DIABODIES | Escalation Study of Duvortuxizumab | |
| | THAT ARE CAPABLE OF | in Participants With Relapsed or | |
| | BINDING CD19 AND CD3, AND | Refractory B-cell Malignancies | |
| | USES THEREOF | AACR 2017 Potent antitumor | |
| | 2015 WO2016048938 | activity of duvortuxizumab, a CD19 × | |
| | JOHNSON, Leslie, . . . BI-SPECIFIC | CD3 DART ® molecule, in | |
| | MONOVALENT DIABODIES | lymphoma models Liat Izhak, Dana | |
| | THAT ARE CAPABLE OF | . . . | |
| | BINDING CD19 AND CD3, AND | ASH 2014 MGD011, Humanized | |
| | USES THEREOF | CD19 × CD3 DART ® Protein with | |
| | | Enhanced Pharmacokinetic | |
| | | Properties, Demonstrates Potent T- | |
| | | Cell Mediated Anti-Tumor Activity | |
| | | in Preclinical Models and Durable B- | |
| | | Cell Depletion in Cynomolgus | |
| | | Monkeys Following Once-a-Week | |
| | | Dosing Liqin Liu, Ph.D., . . . | |
| Elpis Bio patent | 2022 WO2022159653 CHEN, | | Elpis Bio |
| anti-CD19/CD22 | Yan, NGUYEN . . . BISPECIFIC | | |
| | CHIMERIC ANTIGEN | | |
| | RECEPTORS BINDING TO CD19 | | |
| | AND CD22 | | |
| Excyte patent | 2020 WO2020186974 YUAN, | | Excyte |
| anti-CD19/CD3 | Andy Qingan . . . BISPECIFIC | | |
| | ANTIBODY, PREPARATION | | |
| | METHOD THEREFOR AND | | |
| | APPLICATION THEREOF | | |
| | 2020 US20220002408 YUAN, | | |
| | Andy Qingan . . . BISPECIFIC | | |
| | ANTIBODY, PREPARATION | | |
| | METHOD THEREOF AND | | |
| | APPLICATION THEREOF | | |
| FIBH12O patent | 2021 WO2021165248 ÁLVAREZ | | FIBH12O |
| anti-CD19/CD3 | VALLINA, . . . ANTI-CD19/ANTI- | | |
| | CD3 BISPECIFIC ANTIBODY, T | | |
| | CELLS SECRETING THE SAME, | | |
| | METHOD OF PREPARATION | | |
| | AND USE THEREOF | | |
| Gmax Biopharm | 2018 WO2018188612 ZHANG, | | Gmax Biopharm |
| patent anti-CD19/ | Hua, [symbol] , Z . . . BISPECIFIC | | |
| CD3 | ANTIBODY CAPABLE OF BEING | | |
| | COMBINED WITH HUMAN | | |
| | CD19 OR CD20 AND HUMAN | | |
| | CD3, AND APPLICATIONS | | |
| | THEREOF | | |
| Guangdong | 2020 WO2022011846 LI, Peng, | | Guangdong |
| Zhaotai Invivo Bio | QIN, Le . . . CD19- AND CD22- | | Zhaotai Invivo Bio |
| | TARGETED CHIMERIC ANTIGEN | | |
| patent anti-CD19/ | RECEPTOR AND APPLICATION | | |
| CD22 CAR | THEREOF | | |
| IBC Generium | 2020 WO2021034227 | | IBC Generium |
| patent anti-CD3/ | PISKUNOV, Aleksan . . . | | |
| CD19 | COMPLEMENTARITY- | | |
| | DETERMINING REGIONS FOR | | |
| | BINDING CD3, AND BISPECIFIC | | |
| | ANTIGEN-BINDING MOLECULE | | |
| | CONTAINING SAID CDRS | | |
| | 2016 WO2017095267 | | |
| | VICHNEVSKY, Aleks . . . | | |
| | BISPECIFIC ANTIBODIES | | |
| | AGAINST CD3*CD19 | | |
| Immunomedics | 2017 US20170247417 Chang, | ASH 2012 A Novel T-Cell Redirecting | Immunomedics |
| (19)-3s | Chien-Hsin . . . T-Cell Redirecting | Anti-CD19-F(ab)2/CD3scFv | |
| | Bispecific Antibodies for | Bispecific Antibody Exhibits Potent | |
| | Treatment of Disease | Lymphoma Cytotoxicity Diane L | |
| | 2017 U.S. Pat. No. 10,183,992 Chang, | Rossi, M . . . | |
| | Chien-Hsin . . . T-cell redirecting | | |
| | bispecific antibodies for | | |
| | treatment of disease | | |
| | 2016 US20170022274 Chang, | | |
| | Chien-Hsin . . . T-Cell Redirecting | | |
| | Bispecific Antibodies for | | |
| | Treatment of Disease | | |
| | 2016 U.S. Pat. No. 10,239,938 Chang, | | |
| | Chien-Hsin . . . T-cell redirecting | | |
| | bispecific antibodies for | | |
| | treatment of disease | | |
| | 2016 US20170021017 Chang, | | |
| | Chien-Hsin . . . COMBINATION | | |
| | THERAPY FOR INDUCING | | |
| | IMMUNE RESPONSE TO | | |
| | DISEASE | | |
| | 2016 U.S. Pat. No. 10,111,954 Chang, | | |
| | Chien-Hsin . . . Combination | | |
| | therapy for inducing immune | | |
| | response to disease | | |
| | 2016 US20160176980 Chang, | | |
| | Chien-Hsin . . . T-Cell Redirecting | | |
| | Bispecific Antibodies for | | |
| | Treatment of Disease | | |
| | 2016 U.S. Pat. No. 10,308,688 Chang, | | |
| | Chien-Hsin . . . T-cell redirecting | | |
| | bispecific antibodies for | | |
| | treatment of disease | | |
| | 2016 US20160176968 Chang, | | |
| | Chien-Hsin . . . T-Cell Redirecting | | |
| | Bispecific Antibodies for | | |
| | Treatment of Disease | | |
| | 2013 US20140099254 Chang, | | |
| | Chien-Hsin . . . COMBINATION | | |
| | THERAPY FOR INDUCING | | |
| | IMMUNE RESPONSE TO | | |
| | DISEASE | | |
| | 2013 WO2014163684 CHANG, | | |
| | Chien-Hsin . . . COMBINATION | | |
| | THERAPY FOR INDUCING | | |
| | IMMUNE RESPONSE TO | | |
| | DISEASE | | |
| | 2013 U.S. Pat. No. 9,682,143 Chang, Chien- | | |
| | Hsin . . . Combination therapy for | | |
| | inducing immune response to | | |
| | disease | | |
| | 2013 US20140050660 Chang, | | |
| | Chien-Hsin . . . T-Cell Redirecting | | |
| | Bispecific Antibodies for | | |
| | Treatment of Disease | | |
| | 2013 WO2014028560 CHANG, | | |
| | Chien-Hsin . . . T-CELL | | |
| | REDIRECTING BISPECIFIC | | |
| | ANTIBODIES FOR TREATMENT | | |
| | OF DISEASE | | |
| | 2013 U.S. Pat. No. 9,315,567 Chang, Chien- | | |
| | Hsin . . . T-cell redirecting | | |
| | bispecific antibodies for | | |
| | treatment of disease | | |
| Innate patent | 2015 US20170210802 | | Innate |
| anti-CD3/CD19 | GAUTHIER, Laurent . . . | | |
| | MULTISPECIFIC ANTIGEN | | |
| | BINDING PROTEINS | | |
| | 2015 U.S. Pat. No. 11,208,480 Gauthier, | | |
| | Laurent . . . Multispecific antigen | | |
| | binding proteins | | |
| Lentigen patent | 2019 US20200123254 | | Lentigen |
| anti-CD19/CD20 | Schneider, Dina; . . . | | |
| CAR | Compositions and Methods for | | |
| | Treating Cancer with Anti-CD19 | | |
| | Immunotherapy | | |
| | 2019 US20200109210 Orentas, | | |
| | Rimas J . . . COMPOSITIONS AND | | |
| | METHODS FOR TREATING | | |
| | CANCER WITH ANTI- | | |
| | CD19/CD20 IMMUNOTHERAPY | | |
| | 2018 WO2019055842 | | |
| | SCHNEIDER, Dina, . . . | | |
| | COMPOSITIONS AND | | |
| | METHODS FOR TREATING | | |
| | CANCER WITH ANTI-CD19 | | |
| | IMMUNOTHERAPY | | |
| | 2018 US20190106492 | | |
| | Schneider, Dina; . . . | | |
| | Compositions and Methods For | | |
| | Treating Cancer With Anti- | | |
| | CD19 Immunotherapy | | |
| | 2018 U.S. Pat. No. 10,501,539 Schneider, | | |
| | Dina ( . . . Compositions and | | |
| | methods for treating cancer | | |
| | with anti-CD19 immunotherapy | | |
| | 2018 US20180355052 Orentas, | | |
| | Rimas J . . . COMPOSITIONS AND | | |
| | METHODS FOR TREATING | | |
| | CANCER WITH ANTI- | | |
| | CD19/CD20 IMMUNOTHERAPY | | |
| | 2018 WO2019028051 | | |
| | ORENTAS, Rimas J . . . | | |
| | COMPOSITIONS AND | | |
| | METHODS FOR TREATING | | |
| | CANCER WITH ANTI- | | |
| | CD19/CD20 IMMUNOTHERAPY | | |
| | 2018 U.S. Pat. No. 10,442,867 Orentas, | | |
| | Rimas J . . . Compositions and | | |
| | methods for treating cancer | | |
| | with anti-CD19/CD20 | | |
| | immunotherapy | | |
| Lentigen patent | 2022 US20220324967 | | Lentigen NIH |
| anti-CD22/CD19 | Schneider, Dina; . . . | | |
| CAR | Compositions and Methods for | | |
| | Treating Cancer with Anti- | | |
| | CD19/CD22 Immunotherapy | | |
| | 2021 WO2022099026 | | |
| | SCHNEIDER, Dina, . . . | | |
| | COMPOSITIONS AND | | |
| | METHODS FOR TREATING | | |
| | CANCER WITH ANTI- | | |
| | CD19/CD22 IMMUNOTHERAPY | | |
| | 2021 US20210379110 | | |
| | Schneider, Dina; . . . | | |
| | Compositions and Methods for | | |
| | Treating Cancer with TSLPR- | | |
| | CD19 or TSLPR-CD22 | | |
| | Immunotherapy | | |
| | 2020 US20210171633 | | |
| | Schneider, Dina; . . . | | |
| | Compositions and Methods for | | |
| | Treating Cancer with Anti- | | |
| | CD19/CD22 Immunotherapy | | |
| | 2020 U.S. Pat. No. 11,242,389 Schneider, | | |
| | Dina ( . . . Compositions and | | |
| | methods for treating cancer | | |
| | with anti-CD19/CD22 | | |
| | immunotherapy | | |
| | 2020 US20210171632 | | |
| | Schneider, Dina; . . . | | |
| | Compositions and Methods for | | |
| | Treating Cancer with Anti- | | |
| | CD19/CD22 Immunotherapy | | |
| | 2020 US20200155661 Orentas, | | |
| | Rimas J . . . Compositions and | | |
| | Methods For Treating Cancer | | |
| | With Anti-CD22 | | |
| | Immunotherapy | | |
| | 2019 US20200087396 | | |
| | Schneider, Dina; | | |
| | Compositions and Methods for | | |
| | Treating Cancer with Anti- | | |
| | CD19/CD22 Immunotherapy | | |
| | 2019 WO2020069184 | | |
| | SCHNEIDER, Dina, . . . | | |
| | COMPOSITIONS AND | | |
| | METHODS FOR TREATING | | |
| | CANCER WITH ANTI- | | |
| | CD19/CD22 IMMUNOTHERAPY | | |
| | 2019 U.S. Pat. No. 10,822,412 Schneider, | | |
| | Dina ( . . . Compositions and | | |
| | methods for treating cancer | | |
| | with anti-CD19/CD22 | | |
| | immunotherapy | | |
| | 2018 US20190099476 Orentas, | | |
| | Rimas J . . . Compositions and | | |
| | Methods For Treating Cancer | | |
| | With Anti-CD22 | | |
| | Immunotherapy | | |
| | 2018 WO2019079249 | | |
| | ORENTAS, Rimas J . . . | | |
| | COMPOSITIONS AND | | |
| | METHODS FOR TREATING | | |
| | CANCER WITH ANTI-CD22 | | |
| | IMMUNOTHERAPY | | |
| | 2018 U.S. Pat. No. 10,543,263 Orentas, | | |
| | Rimas J . . . Compositions and | | |
| | methods for treating cancer | | |
| | with anti-CD22 immunotherapy | | |
| Lentigen patent | 2021 WO2021262723 | | Lentigen |
| anti-TSLPR/CD19 | SCHNEIDER, Dina, . . . | | U. Colorado |
| CAR | COMPOSITIONS AND | | |
| | METHODS FOR TREATING | | |
| | CANCER WITH TSLPR-CD19 OR | | |
| | TSLPR-CD22 IMMUNOTHERAPY | | |
| | 2021 U.S. Pat. No. 11,497,770 Schneider, | | |
| | Dina; . . . Compositions and | | |
| | methods for treating cancer | | |
| | with TSLPR-CD19 or TSLPR- | | |
| | CD22 immunotherapy | | |
| Mass. General | 2019 WO2020124021 MAUS, | | Mass. General |
| Hosp. patent anti- | Marcela, V. CHIMERIC ANTIGEN | | Hosp. |
| CD19/CD79b | RECEPTORS TARGETING CD79B | | |
| CAR | AND CD19 | | |
| MB-CART2019.1 | | 2021 NCT04844866 Phase 2 Efficacy | Miltenyi Biotec |
| | | and Safety of MB-CART2019.1 vs. | |
| | | SoC in Lymphoma Patients | |
| Nanjing Iaso Bio | 2021 WO2022002154 YANG, | | Iaso |
| patent anti-CD22/ | Yongkun, H U . . . FULLY | | |
| CD19 CAR | HUMANIZED BISPECIFIC | | |
| | CHIMERIC ANTIGEN RECEPTOR | | |
| | TARGETING CD19 AND CD22 | | |
| | AND USE THEREOF | | |
| NIH anti- | | 2021 Mhibik M, Gaglion . . . BTK | NIH |
| CD19/CD3 | | inhibitors, irrespective of ITK | |
| | | inhibition, increase efficacy of a | |
| | | CD19/CD3-bispecific antibody in | |
| | | CLL. | |
| | | 2018 Robinson H R, Qi J . . . A | |
| | | CD19/CD3 bispecific antibody for | |
| | | effective immunotherapy of chronic | |
| | | lymphocytic leukemia in the | |
| | | ibrutinib era. | |
| NIH patent anti- | 2019 WO2020014482 | | NIH |
| CD19/CD22 CAR | DIMITROV, Dimiter . . . AFFINITY | | |
| | MATURED CD22-SPECIFIC | | |
| | MONOCLONAL ANTIBODY AND | | |
| | USES THEREOF | | |
| | 2016 WO2016149578 FRY, | | |
| | Terry J., MA . . . DUAL SPECIFIC | | |
| | ANTI-CD22-ANTI-CD19 | | |
| | CHIMERIC ANTIGEN | | |
| | RECEPTORS | | |
| Ono patent anti- | 2020 WO2020204152 | | Ono |
| PD-1/CD19 | SHIBAYAMA, Shiro, . . . | | |
| | BISPECIFIC ANTIBODY | | |
| | 2020 US20220185885 | | |
| | SHIBAYAMA, Shiro; . . . | | |
| | BISPECIFIC ANTIBODY | | |
| | 2018 US20200317783 | | |
| | SHIBAYAMA, Shiro; . . . | | |
| | BISPECIFIC ANTIBODY | | |
| RO7227166 | 2022 US20220259327 Amann, | 2019 NCT04077723 Phase 1 A Study | Roche |
| | Maria; Bru . . . ANTIGEN BINDING | to Evaluate the Safety, | |
| | MOLECULES COMPRISING A | Pharmacokinetics and Preliminary | |
| | TNF FAMILY LIGAND TRIMER | Anti-Tumor Activity of RO7227166 | |
| | 2022 US20220259326 Amann, | in Combination With Obinutuzumab | |
| | Maria; Bru . . . ANTIGEN BINDING | and in Combination With CD20-TCB | |
| | MOLECULES COMPRISING A | Following a Pre-Treatment Dose of | |
| | TNF FAMILY LIGAND TRIMER | Obinutuzumab Administered in | |
| | 2020 US20210253724 CLAUS, | Participants With Relapse . . . | |
| | Christina; . . . NOVEL BISPECIFIC | | |
| | AGONISTIC 4-1BB ANTIGEN | | |
| | BINDING MOLECULES | | |
| | 2020 US20210163617 FERRARA | | |
| | KOLLER, C . . . THERAPEUTIC | | |
| | COMBINATION OF 4-1 BB | | |
| | AGONISTS WITH ANTI-CD20 | | |
| | ANTIBODIES | | |
| | 2019 US20200190206 FERRARA | | |
| | KOLLER, C . . . BISPECIFIC | | |
| | ANTIGEN BINDING MOLECULES | | |
| | COMPRISING ANTI-4-1BB | | |
| | CLONE 20H4.9 | | |
| | 2019 U.S. Pat. No. 11,447,558 Ferrara | | |
| | Koller, C . . . Bispecific antigen | | |
| | binding molecules comprising | | |
| | anti-4-1BB clone 20H4.9 | | |
| | 2019 WO2019175071 FERRARA | | |
| | KOLLER, C . . . THERAPEUTIC | | |
| | COMBINATION OF 4-1 BB | | |
| | AGONISTS WITH ANTI-CD20 | | |
| | ANTIBODIES | | |
| Roche patent | 2021 WO2021255155 | | Roche |
| anti-CD19/CD3 | FREIMOSER-GRUNDSC . . . | | |
| | ANTIBODIES BINDING TO CD3 | | |
| | AND CD19 | | |
| | 2020 US20210070882 BACAC, | | |
| | Marina; KL . . . BISPECIFIC T CELL | | |
| | ACTIVATING ANTIGEN BINDING | | |
| | MOLECULES | | |
| | 2016 US20170174786 Bacac, | | |
| | Marina; Kl . . . BISPECIFIC T CELL | | |
| | ACTIVATING ANTIGEN BINDING | | |
| | MOLECULES | | |
| | 2016 WO2017055314 BACAC, | | |
| | Marina, KL . . . BISPECIFIC ANTI- | | |
| | CD19 × CD3 T CELL ACTIVATING | | |
| | ANTIGEN BINDING MOLECULES | | |
| Shenzhen | 2022 WO2022194264 WEN, Yu, | | Shenzhen |
| Enduring Bio | LIU, Shu . . . PEGYLATED T CELL | | Enduring Bio |
| patent anti-CD3/ | ENGAGER WITH DUAL | | |
| CD19 | SPECIFICITIES TO CD3 AND | | |
| | CD19 | | |
| Sichuan Kelun | 2021 WO2022007650 CHANG, | | Sichuan Kelun |
| patent anti-BCMA/ | Jianhui, Z . . . CHIMERIC ANTIGEN | | Pharma |
| CD19 CAR | RECEPTOR CAR OR CAR | | |
| | CONSTRUCT TARGETING BCMA | | |
| | AND CD19 AND APPLICATION | | |
| | THEREOF | | |
| Sichuan University | | 2012 Zhou Y, Gou L T, M . . . A fully | Sichuan |
| anti-CD19/CD3 | | human CD19/CD3 bi-specific | University |
| ScFv | | antibody triggers potent and | |
| | | specific cytotoxicity by | |
| | | unstimulated T lymphocytes against | |
| | | non-Hodgkin's lymphoma. | |
| TG-1801 | 2018 US20180291115 | 2021 NCT04806035 Phase 1 Study | NovImmune TG |
| | MASTERNAK, Krzysz . . . ANTI- | of TG-1801 Alone or in Combination | Therapeutics |
| | CD47 ANTIBODIES AND | With Ublituximab in Subjects With | |
| | METHODS OF USE THEREOF | B-Cell Lymphoma or Chronic | |
| | 2018 US20180291116 | Lymphocytic Leukemia | |
| | MASTERNAK, Krzysz . . . ANTI- | 2019 NCT03804996 Phase 1 Study | |
| | CD47 ANTIBODIES AND | of TG-1801 in Subjects With B-Cell | |
| | METHODS OF USE THEREOF | Lymphoma | |
| | 2013 US20140303354 | 2022 Chauchet X, Cons . . . | |
| | Masternak, Krzysz . . . Anti-CD47 | CD47 × CD19 bispecific antibody | |
| | Antibodies and Methods of Use | triggers recruitment and activation | |
| | Thereof | of innate immune effector cells in a | |
| | | B-cell lymphoma xenograft model. | |
| | | 2018 Buatois V, Johnso . . . Preclinical | |
| | | development of a bispecific | |
| | | antibody that safely and effectively | |
| | | targets CD19 and CD47 for the | |
| | | treatment of B cell lymphoma and | |
| | | leukemia. | |
| | | 2017 Dheilly E, Moine . . . Selective | |
| | | Blockade of the Ubiquitous | |
| | | Checkpoint Receptor CD47 Is | |
| | | Enabled by Dual-Targeting Bispecific | |
| | | Antibodies. | |
| | | AACR 2015 Neutralizing CD47 in | |
| | | cancer cells with dual targeting | |
| | | kappa/lambda bodies Krzysztof | |
| | | Mastern . . . | |
| | | ASCO 2015 Bispecific antibody | |
| | | targeting of CD47/CD19 to promote | |
| | | enhanced phagocytosis of patient B | |
| | | lymphoma cells Zoe Johnson, | |
| | | Anne . . . | |
| | | ASH 2016 A CD47 × CD19 Bispecific | |
| | | Antibody That Remodels the Tumor | |
| | | Microenvironment for Improved | |
| | | Killing and Provokes a Memory | |
| | | Immune Response to Cancer B Cells | |
| | | Walter G. Ferlin, . . . | |
| TNB-486 | | 2020 NCT04594642 Phase 1 A Study | TeneoBio |
| | | of TNB-486 in Subjects With | |
| | | Relapsed or Refractory B-Cell Non- | |
| | | Hodgkin Lymphoma | |
| | | ASH 2019 TNB-486, a Novel Fully | |
| | | Human Bispecific CD19 × CD3 | |
| | | Antibody That Kills CD19-Positive | |
| | | Tumor Cells with Minimal Cytokine | |
| | | Secretion Harbani Malik, Ph . . . | |
| TT19 | 2017 US20200207851 CHEN, | | CytoCares |
| | Shuai; ZHU, . . . TRIFUNCTIONAL | | |
| | MOLECULE AND APPLICATION | | |
| | THEREOF | | |
| WuXi Biologics | 2018 WO2019057122 XU, | | WuXi Biologics |
| patent anti-CD19/ | Jianqing, WAN . . . NOVEL | | |
| CD3 | BISPECIFIC POLYPEPTIDE | | |
| | COMPLEXES | | |
| | 2018 WO2019057124 LIU, | | |
| | Jieying, XU, . . . NOVEL BISPECIFIC | | |
| | CD3/CD19 POLYPEPTIDE | | |
| | COMPLEXES | | |
| ZW38 | 2015 WO2015109131 NG, | ASCO 2015 ZW38, a bispecific CD3 × | Zymeworks |
| | Gordon, Yiu K . . . BI-SPECIFIC | CD19 azymetric antibody to deplete | |
| | CD3 AND CD19 ANTIGEN- | human leukemic B cells by the | |
| | BINDING CONSTRUCTS | “controlled” activation of T cells | |
| | 2015 US20160326249 Ng, | Gordon Ng, Nina W . . . | |
| | Gordon Yiu Ko . . . BI-SPECIFIC | ASH 2016 ZW38, a Novel Azymetric | |
| | CD3 AND CD19 ANTIGEN- | Bispecific CD19-Directed CD3 T Cell | |
| | BINDING CONSTRUCTS | Engager Antibody Drug Conjugate | |
| | 2014 US20160355588 Ng, | with Controlled T Cell Activation | |
| | Gordon Yiu Ko . . . Bispecific CD3 | and Improved B Cell Cytotoxicity | |
| | and CD19 Antigen Binding | Gordon Ng, PhD, M . . . | |
| | Constructs | | |
| A-2019 | | 2021 Wang S, Peng L, X . . . Preclinical | Generon |
| | | characterization and comparison | |
| | | between CD3/CD19 bispecific and | |
| | | novel CD3/CD19/CD20 trispecific | |
| | | antibodies against B-cell acute | |
| | | lymphoblastic leukemia: targeted | |
| | | immunotherapy for acute | |
| | | lymphoblastic leukemia. | |
| Affimed patent- | 2018 US20190040155 | | Affimed |
| CD30/CD19/CD16A | ELLWANGER, Kristi . . . | | |
| | MULTIVALENT FV ANTIBODIES | | |
| | 2016 WO2017064221 | | |
| | ELLWANGER, Kristi . . . | | |
| | MULTIVALENT FV ANTIBODIES | | |
| CMG1A46 | | 2022 NCT05348889 Phase 1/Phase | Chimagen |
| | | 2 First-in-Human (FIH) Trial of 1A46 | |
| | | in Subjects With Advanced CD20 | |
| | | and/or CD19 Positive B-cell | |
| | | Hematologic Malignancies | |
| Cullinan Oncology | 2020 WO2021119551 | | Cullinan |
| patent anti-CD19/ | BAEUERLE, Patrick . . . ANTI-CD19 | | Oncology |
| CD3/serum | ANTIBODIES AND MULTI- | | |
| albumin | SPECIFIC BINDING PROTEINS | | |
| | 2020 US20230037815 | | |
| | Baeuerle, Patrick . . . ANTI-CD19 | | |
| | ANTIBODIES AND MULTI- | | |
| | SPECIFIC BINDING PROTEINS | | |
| | 2019 WO2019237081 | | |
| | BAEUERLE, Patrick . . . MULTI- | | |
| | SPECIFIC BINDING PROTEINS | | |
| | AND METHODS OF USE | | |
| | THEREOF | | |
| German CRC | 2019 WO2020053300 JUNG, | | Eberhard-Karls U. |
| patent anti-CD19/ | Gundram, SA . . . IMPROVED | | German CRC |
| FLT3/CD3 | ANTI-FLT3 ANTIGEN BINDING | | |
| | PROTEINS | | |
| | 2019 US20220056141 Jung, | | |
| | Gundram; Sa . . . Improved Anti- | | |
| | FLT3 Antigen Binding Proteins | | |
| | 2015 WO2016023909 JUNG, | | |
| | Gundram, S . . . RECOMBINANT | | |
| | ANTIBODY MOLECULE AND ITS | | |
| | USE FOR TARGET CELL | | |
| | RESTRICTED T CELL | | |
| | ACTIVATION | | |
| Novartis patent | 2021 WO2022097061 | | Novartis |
| anti-CD20/CD19/ | AARDALEN, Kimberl . . . ANTI- | | |
| CD3 | CD19 AGENT AND B CELL | | |
| | TARGETING AGENT | | |
| | COMBINATION THERAPY FOR | | |
| | TREATING B CELL | | |
| | MALIGNANCIES | | |
| | 2021 WO2022097060 CEBE, | | |
| | Regis, CHEL . . . CD19 BINDING | | |
| | MOLECULES AND USES | | |
| | THEREOF | | |
| | 2020 WO2020236792 | | |
| | GRANDA, Brian, RA . . . CD19 | | |
| | BINDING MOLECULES AND | | |
| | USES THEREOF | | |
| | 2019 WO2019195535 | | |
| | GRANDA, Brian, HO . . . | | |
| | TRISPECIFIC BINDING | | |
| | MOLECULES AGAINST CANCERS | | |
| | AND USES THEREOF | | |
| Peking U. anti- | | 2022 Zhao L, Li S, Wei . . . A novel | Peking U. |
| CD19/CD22/CD3 | | CD19/CD22/CD3 trispecific | |
| | | antibody enhances therapeutic | |
| | | efficacy and overcomes immune | |
| | | escape against B-ALL. | |

b. CD20 CAR

In some embodiments, the additional CAR is a CD20 CAR (“CD20-CAR”), and in these embodiments, the polycistronic vector comprises an expression cassette that contains a nucleotide sequence encoding a CD20 CAR. CD20 is an antigen found on the surface of B cells as early at the pro-B3 phase and progressively at increasing levels until B cell maturity, as well as on the cells of most B-cell neoplasms. CD20 positive cells are also sometimes found in cases of Hodgkins disease, myeloma, and thymoma. In some embodiments, the CD20 CAR may comprise a signal peptide, an extracellular binding domain that specifically binds CD20, a hinge domain, a transmembrane domain, an intracellular costimulatory domain, and/or an intracellular signaling domain in tandem.

In some embodiments, the signal peptide of the CD20 CAR comprises a CD8a signal peptide. In some embodiments, the CD8a signal peptide comprises or consists of an amino acid sequence set forth in SEQ ID NO:6 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:6. In some embodiments, the signal peptide comprises an IgK signal peptide. In some embodiments, the IgK signal peptide comprises or consists of an amino acid sequence set forth in SEQ ID NO:7 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:7. In some embodiments, the signal peptide comprises a GMCSFR-α or CSF2RA signal peptide. In some embodiments, the GMCSFR-α or CSF2RA signal peptide comprises or consists of an amino acid sequence set forth in SEQ ID NO:8 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:8.

In some embodiments, the extracellular binding domain of the CD20 CAR is specific to CD20, for example, human CD20. The extracellular binding domain of the CD20 CAR can be codon-optimized for expression in a host cell or to have variant sequences to increase functions of the extracellular binding domain. In some embodiments, the extracellular binding domain comprises an immunogenically active portion of an immunoglobulin molecule, for example, an scFv.

In some embodiments, the extracellular binding domain of the CD20 CAR is derived from an antibody specific to CD20, including, for example, Leu16, IF5, 1.5.3, rituximab, obinutuzumab, ibritumomab, ofatumumab, tositumumab, odronextamab, veltuzumab, ublituximab, and ocrelizumab. In any of these embodiments, the extracellular binding domain of the CD20 CAR can comprise or consist of the V_{H}, the V_{L}, and/or one or more CDRs of any of the antibodies.

In some embodiments, the extracellular binding domain of the CD20 CAR comprises an scFv derived from the Leu16 monoclonal antibody, which comprises the heavy chain variable region (V_{H}) and the light chain variable region (V_{L}) of Leu16 connected by a linker. See Wu et al., Protein Engineering. 14(12):1025-1033 (2001). In some embodiments, the linker is a 3×G₄S linker. In other embodiments, the linker is a Whitlow linker as described herein. In some embodiments, the amino acid sequences of different portions of the entire Leu16-derived scFv (also referred to as Leu16 scFv) and its different portions are provided in Table 13 below. In some embodiments, the CD20-specific scFv comprises or consists of an amino acid sequence set forth in SEQ ID NO:37, 38, or 42, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:37, 38, or 42. In some embodiments, the CD20-specific scFv may comprise one or more CDRs having amino acid sequences set forth in SEQ ID NOs: 39-41, 43-44 and 107. In some embodiments, the CD20-specific scFv may comprise a light chain with one or more CDRs having amino acid sequences set forth in SEQ ID NOs: 39-41. In some embodiments, the CD20-specific scFv may comprise a heavy chain with one or more CDRs having amino acid sequences set forth in SEQ ID NOs: 43-44 and 107. In any of these embodiments, the CD20-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the CD20 CAR comprises or consists of the one or more CDRs as described herein.

TABLE 13
Exemplary sequences of anti-CD20 scFv and components

| SEQ ID NO: | Amino Acid Sequence | Description |
|---|---|---|
| 37 | DIVLTQSPAILSASPGEKVTMTCRASSSVNYMD | Anti-CD20 Leu16 scFv |
| | WYQKKPGSSPKPWIYATSNLASGVPARFSGSGS | entire sequence, with |
| | GTSYSLTISRVEAEDAATYYCQQWSFNPPTFGG | Whitlow linker |
| | GTKLEIKGSTSGSGKPGSGEGSTKGEVQLQQSGA | |
| | ELVKPGASVKMSCKASGYTFTSYNMHWVKQTP | |
| | GQGLEWIGAIYPGNGDTSYNQKFKGKATLTADK | |
| | SSSTAYMQLSSLTSEDSADYYCARSNYYGSSYWF | |
| | FDVWGAGTTVTVSS | |
| 38 | DIVLTQSPAILSASPGEKVTMTCRASSSVNYMD | Anti-CD20 Leu16 scFv light |
| | WYQKKPGSSPKPWIYATSNLASGVPARFSGSGS | chain variable region |
| | GTSYSLTISRVEAEDAATYYCQQWSFNPPTFGG | |
| | GTKLEIK | |
| 39 | RASSSVNYMD | Anti-CD20 Leu16 scFv light |
| | | chain CDR1 |
| 40 | ATSNLAS | Anti-CD20 Leu16 scFv light |
| | | chain CDR2 |
| 41 | QQWSFNPPT | Anti-CD20 Leu16 scFv light |
| | | chain CDR3 |
| 42 | EVQLQQSGAELVKPGASVKMSCKASGYTFTSYN | Anti-CD20 Leu16 scFv |
| | MHWVKQTPGQGLEWIGAIYPGNGDTSYNQKF | heavy chain |
| | KGKATLTADKSSSTAYMQLSSLTSEDSADYYCAR | |
| | SNYYGSSYWFFDVWGAGTTVTVSS | |
| 43 | SYNMH | Anti-CD20 Leu16 scFv |
| | | heavy chain CDR1 |
| 44 | AIYPGNGDTSYNQKFKG | Anti-CD20 Leu16 scFv |
| | | heavy chain CDR2 |
| 107 | SNYYGSSYWFFDV | 107 |

In some embodiments, the hinge domain of the CD20 CAR comprises a CD8a hinge domain, for example, a human CD8a hinge domain. In some embodiments, the CD8a hinge domain comprises or consists of an amino acid sequence set forth in SEQ ID NO:9 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:9. In some embodiments, the hinge domain comprises a CD28 hinge domain, for example, a human CD28 hinge domain. In some embodiments, the CD28 hinge domain comprises or consists of an amino acid sequence set forth in SEQ ID NO:10 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:10. In some embodiments, the hinge domain comprises an IgG4 hinge domain, for example, a human IgG4 hinge domain. In some embodiments, the IgG4 hinge domain comprises or consists of an amino acid sequence set forth in SEQ ID NO:11 or SEQ ID NO:12, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:11 or SEQ ID NO:12. In some embodiments, the hinge domain comprises a IgG4 hinge-Ch2-Ch3 domain, for example, a human IgG4 hinge-Ch2-Ch3 domain. In some embodiments, the IgG4 hinge-Ch2-Ch3 domain comprises or consists of an amino acid sequence set forth in SEQ ID NO:13 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:13.

In some embodiments, the transmembrane domain of the CD20 CAR comprises a CD8a transmembrane domain, for example, a human CD8a transmembrane domain. In some embodiments, the CD8a transmembrane domain comprises or consists of an amino acid sequence set forth in SEQ ID NO:14 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:14. In some embodiments, the transmembrane domain comprises a CD28 transmembrane domain, for example, a human CD28 transmembrane domain. In some embodiments, the CD28 transmembrane domain comprises or consists of an amino acid sequence set forth in SEQ ID NO:15 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:15.

In some embodiments, the intracellular costimulatory domain of the CD20 CAR comprises a 4-1BB costimulatory domain, for example, a human 4-1BB costimulatory domain. In some embodiments, the 4-1BB costimulatory domain comprises or consists of an amino acid sequence set forth in SEQ ID NO:16 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:16. In some embodiments, the intracellular costimulatory domain comprises a CD28 costimulatory domain, for example, a human CD28 costimulatory domain. In some embodiments, the CD28 costimulatory domain comprises or consists of an amino acid sequence set forth in SEQ ID NO:17 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:17.

In some embodiments, the intracellular signaling domain of the CD20 CAR comprises a CD3 zeta (ζ) signaling domain, for example, a human CD3ζ signaling domain. In some embodiments, the CD3ζ signaling domain comprises or consists of an amino acid sequence set forth in SEQ ID NO:18 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:18.

In some embodiments, the polycistronic vector comprises an expression cassette that contains a nucleotide sequence encoding a CD20 CAR, including, for example, a CD20 CAR comprising the CD20-specific scFv having sequences set forth in SEQ ID NO:37, the CD8a hinge domain of SEQ ID NO:9, the CD8a transmembrane domain of SEQ ID NO:14, the 4-1BB costimulatory domain of SEQ ID NO:16, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD20 CAR, including, for example, a CD20 CAR comprising the CD20-specific scFv having sequences set forth in SEQ ID NO:37, the CD28 hinge domain of SEQ ID NO:10, the CD8a transmembrane domain of SEQ ID NO:14, the 4-1BB costimulatory domain of SEQ ID NO:16, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD20 CAR, including, for example, a CD20 CAR comprising the CD20-specific scFv having sequences set forth in SEQ ID NO:37, the IgG4 hinge domain of SEQ ID NO:11 or SEQ ID NO:12, the CD8a transmembrane domain of SEQ ID NO:14, the 4-1BB costimulatory domain of SEQ ID NO:16, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD20 CAR, including, for example, a CD20 CAR comprising the CD20-specific scFv having sequences set forth in SEQ ID NO:37, the CD8a hinge domain of SEQ ID NO:9, the CD28 transmembrane domain of SEQ ID NO:15, the 4-1BB costimulatory domain of SEQ ID NO:16, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD20 CAR, including, for example, a CD20 CAR comprising the CD20-specific scFv having sequences set forth in SEQ ID NO:37, the CD28 hinge domain of SEQ ID NO:10, the CD28 transmembrane domain of SEQ ID NO:15, the 4-1BB costimulatory domain of SEQ ID NO:16, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD20 CAR, including, for example, a CD20 CAR comprising the CD20-specific scFv having sequences set forth in SEQ ID NO:37, the IgG4 hinge domain of SEQ ID NO:11 or SEQ ID NO:1, the CD28 transmembrane domain of SEQ ID NO:15, the 4-1BB costimulatory domain of SEQ ID NO:16, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, a polynucleotide provided herein comprises a nucleotide sequence encoding a CD20 CAR, a variable domain of a CD20 CAR, or a set of CDRs (HCDR 1, 2, and 3 and LCDR 1, 2, and 3) that encodes a CD20 CAR as set forth in TABLE 14 below or a variable domain of a CD20 CAR or a set of CDRs (HCDR 1, 2, and 3 and LCDR 1, 2, and 3) therefrom. In some embodiments, a polynucleotide provided herein comprises a nucleotide sequence encoding a CD20 CAR, a variable domain of a CD20 CAR, or a set of CDRs (HCDR 1, 2, and 3 and LCDR 1, 2, and 3) that having at least 80% (e.g., at least 80%, at least 85%, at least 90%, at least 95%, at least 96%, at least 97%, at least 98%, at least 99%, or 100% identical) to a CD20 CAR as set forth in TABLE 14 below or a variable domain of a CD20 CAR or a set of CDRs (HCDR 1, 2, and 3 and LCDR 1, 2, and 3) therefrom, respectively.

TABLE 14
Exemplary CD20 antigen binding domains

| Antibody Name | Company |
|---|---|
| Academia Sinica patent anti-CD20 | Academia Sinica |
| Avesthagen patent anti-CD20 | Avesthagen |
| B001 | Shanghai Pharma Holdings |
| BAT4306f | Bio-Thera Solutions |
| BCD-020 | Biocad |
| Beijing Abco patent anti-CD20 | Beijing Abco |
| BI 695500 | Boehringer |
| Bioex patent anti-CD20 | Bioex |
| BLX-301 | Biolex |
| BVX20 | Biocon Vaccinex |
| Cellular Biomed. patent anti-CD20 | Cellular Biomed. |
| Chinese PLA Gen. Hosp. patent anti-CD20 CAR | Chinese PLA Gen. Hosp. |
| CHO-H01 | Cho Pharma |
| CMAB304 | Shanghai CP Guojian |
| divozilimab | Biocad |
| Duke U. patent anti-CD20 | Duke U. |
| DXL625 | InNexus |
| DXLr120 | InNexus |
| EDC9 | Centrose |
| Eureka anti-CD20 | Eureka |
| F007 | Shandong New Time Pharma |
| FBT-A05 | Fresenius |
| GB241 | Genor |
| Genmab patent anti-CD20 | Genmab |
| GNR-006 | IBC Generium |
| GP2013 | Novartis Sandoz |
| Haisco Ofatumumab variant | Haisco Pharmaceutical |
| Haixi rituximab biosimilar | Haixi |
| HLX01 | Shanghai Henlius |
| IBI301 | Innovent Lilly |
| ibritumomab tiuxetan | Biogen CTI Biopharma Spectrum |
| IGN002 | ImmunGene Valor Bio |
| Immunogen patent anti-CD20 | ImmunoGen |
| JHL1101 | JHL Biotech |
| KHK patent anti-CD20 | KHK |
| Kyoto U. patent anti-CD20 | Biomedics Kyoto U. |
| mAb 1.5.3 | AstraZeneca |
| MabionCD20 | Mabion |
| MEDI-552 | AstraZeneca Medimmune |
| Medimmune patent anti-CD20 | Medimmune |
| Merck patent anti-CD20 | Merck Serono |
| MIL62 | Beijing Mabworks |
| MK-8808 | Merck (MSD) |
| MRG001 | Shanghai Miracogen |
| MT-3724 | Molecular Templates |
| Nanjing Legend Bio patent anti-CD20 | Nanjing Legend Bio |
| NAV006 | Navrogen |
| Novartis patent anti-CD20 CAR | Novartis |
| obinutuzumab | Biogen Genentech Glycart Roche |
| ocaratuzumab | AME Lilly Mentrik |
| ocrelizumab | Biogen Genentech Xoma |
| OFA-HL-vcMMAE | Zhejiang U. |
| ofatumumab | Genmab GSK Novartis |
| Osaka U. patent anti-CD20 | Osaka U. |
| Palleon patent anti-CD20 | Palleon |
| PBO-326 | Probiomed |
| PBP1506 | Prestige BioPharma |
| Precision Biotech patent anti-CD20 CAR | Precision Biologics |
| PRO131921 | Genentech |
| PSB102 | Sound Biologics |
| Redditux | Dr. Reddy's |
| Reditux | Dr. Reddy's |
| Regeneron patent anti-CD20 | Regeneron |
| RGB-03 | Gedeon Richter |
| ripertamab | Sinocelltech |
| rituximab | Biogen Roche |
| rituximab-abbs | Celltrion Mundipharma |
| rituximab-arrx | Amgen |
| rituximab-pvvr | Pfizer |
| rituximab-vcMMAE | Shahid Beheshti U. Med. Sci. |
| RO7082859 | Roche |
| RTXM83 | mAbxience |
| SAIT101 | Samsung Bioepis |
| SBI-087 | Pfizer Trubion |
| Second Military Med Univ CD20 | Second Military Med Univ |
| Shanghai PAE patent anti-CD20 | Shanghai PAE |
| Sloan-Kettering patent anti-CD20 | Sloan-Kettering |
| Sorrento patent anti-CD20 | Sorrento |
| Sunshine Guojian 304 | Sunshine Guojian Pharma |
| Tabriz U. single chain anti-CD20 | Tabriz U. |
| Tarbiat Modares U. anti-CD20 | Tarbiat Modares U. |
| TeneoBio patent anti-CD20 | TeneoBio |
| TG20 | LFB |
| TL011 | Teva |
| tositumomab | GSK |
| TPI patent anti-CD20 | TPI |
| TQB2303 | Chia Tai Tianqing Pharma |
| TRS005 | Zhejiang Teruisi |
| TRU-015 | Pfizer Trubion |
| UB-923 | United Biopharma |
| ublituximab | LFB TG Therapeutics |
| UMC Utrecht patent anti-CD20 | Tiga TX UMC Utrecht |
| US Navy patent anti-CD20 | US Navy |
| veltuzumab | Immunomedics Nycomed Takeda |
| VIB patent anti-CD20 | VIB Vrije Universiteit Brussel |
| Xencor patent anti-CD20 | Xencor |
| Zhao patent anti-CD20 | |
| Zitux | Aryogen |
| zuberitamab | Zheijang Hisun |
| 2B8T2M | Altor |
| Ampsource patent anti-CD20/CD3 | Ampsource |
| APO | Baliopharm German CRC |
| Beijing Mabworks patent anti-CD3/CD20 | Beijing Mabworks |
| Celgene patent anti-CD47/CD20 | Celgene |
| Cellectis patent anti-CD20/CD22 CAR | Cellectis |
| Cellular Biomed. patent anti-CD19/CD20 CAR | Cellular Biomed. |
| Chinese Mil. Med. Sci. anti-CD20/HLA-DR | Chinese Mil. Med. Sci. |
| CM355 | Beijing Tiannuojiancheng InnoCare Keymed |
| Development Center for Biotech. anti-CD3/CD20 | Development Center for Biotech. |
| epcoritamab | Abbvie Genmab |
| FBTA05 | TRION Pharma |
| Genmab anti-CD20(2)/CD16 | Genmab |
| glofitamab | Roche |
| Guangzhou Excelmab patent anti-CD3/CD20 | Guangzhou Excelmab |
| IGM-2323 | IGM Bio |
| IMM0306 | ImmuneOnco |
| Immunomedics 20-(74)-(74) | Immunomedics |
| Immunomedics 74-(20)-(20) | Immunomedics |
| Lentigen patent anti-CD19/CD20 CAR | Lentigen |
| Lilly patent anti-BAFF/CD20 | Lilly |
| Mab-Legend Bio patent anti-CD3/CD20 | Mab-Legend Bio |
| MB-CART2019.1 | Miltenyi Biotec |
| mosunetuzumab-axgb | Genentech |
| odronextamab | Regeneron Zai Lab |
| plamotamab | Novartis Xencor |
| QLB patent anti-CD3/CD20 | Qilu Pharma |
| Roche patent anti-CD20/Tfr | Roche |
| Scripps anti-CD52/CD20 | Feinstein Inst. Scripps |
| Second Military Med Univ CD20-243 CrossMab | Second Military Med Univ |
| Sloan-Kettering patent anti-EphA2/CD20 | Sloan-Kettering |
| Stanford anti-CD47/CD20 | Stanford |
| WuXi Biologics patent anti-CD20/CD3 | WuXi Biologics |
| Xencor patent anti-CD8/CD20 | Xencor |
| Yang Yang patent anti-CD3/CD20 | |
| Yonsei U. patent anti-CD20/TNFR1 | Yonsei U. |
| A-2019 | Generon |
| Amgen patent anti-CD20/CD22/CD3 | Amgen |
| CMG1A46 | Chimagen |
| Innate patent anti-NKp46/CD20/IL-2R beta | Innate |
| Janssen patent anti-CD79b/CD20/CD3 | Janssen Biotech |
| Novartis patent anti-CD20/CD19/CD3 | Novartis |

c. CD22 CAR

In some embodiments, the CAR is a CD22 CAR (“CD22-CAR”), and in these embodiments, the polycistronic vector comprises an expression cassette that contains a nucleotide sequence encoding a CD22 CAR. CD22, which is a transmembrane protein found mostly on the surface of mature B cells that functions as an inhibitory receptor for B cell receptor (BCR) signaling. CD22 is expressed in 60-70% of B cell lymphomas and leukemias (e.g., B-chronic lymphocytic leukemia, hairy cell leukemia, acute lymphocytic leukemia (ALL), and Burkitt's lymphoma) and is not present on the cell surface in early stages of B cell development or on stem cells. In some embodiments, the CD22 CAR may comprise a signal peptide, an extracellular binding domain that specifically binds CD22, a hinge domain, a transmembrane domain, an intracellular costimulatory domain, and/or an intracellular signaling domain in tandem.

In some embodiments, the signal peptide of the CD22 CAR comprises a CD8a signal peptide. In some embodiments, the CD8a signal peptide comprises or consists of an amino acid sequence set forth in SEQ ID NO:6 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:6. In some embodiments, the signal peptide comprises an IgK signal peptide. In some embodiments, the IgK signal peptide comprises or consists of an amino acid sequence set forth in SEQ ID NO:7 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:7. In some embodiments, the signal peptide comprises a GMCSFR-α or CSF2RA signal peptide. In some embodiments, the GMCSFR-α or CSF2RA signal peptide comprises or consists of an amino acid sequence set forth in SEQ ID NO:8 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:8.

In some embodiments, the extracellular binding domain of the CD22 CAR is specific to CD22, for example, human CD22. The extracellular binding domain of the CD22 CAR can be codon-optimized for expression in a host cell or to have variant sequences to increase functions of the extracellular binding domain. In some embodiments, the extracellular binding domain comprises an immunogenically active portion of an immunoglobulin molecule, for example, an scFv.

In some embodiments, the extracellular binding domain of the CD22 CAR is derived from an antibody specific to CD22, including, for example, SM03, inotuzumab, epratuzumab, moxetumomab, and pinatuzumab. In any of these embodiments, the extracellular binding domain of the CD22 CAR can comprise or consist of the V_{H}, the V_{L}, and/or one or more CDRs of any of the antibodies.

In some embodiments, the extracellular binding domain of the CD22 CAR comprises an scFv derived from the m971 monoclonal antibody (m971), which comprises the heavy chain variable region (V_{H}) and the light chain variable region (V_{L}) of m971 connected by a linker. In some embodiments, the linker is a 3×G₄S linker. In other embodiments, the Whitlow linker may be used instead. In some embodiments, the amino acid sequences of the entire m971-derived scFv (also referred to as m971 scFv) and its different portions are provided in Table 15 below. In some embodiments, the CD22-specific scFv comprises or consists of an amino acid sequence set forth in SEQ ID NO:45, 46, or 50, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:45, 46, or 50. In some embodiments, the CD22-specific scFv may comprise one or more CDRs having amino acid sequences set forth in SEQ ID NOs: 47-49 and 51-53. In some embodiments, the CD22-specific scFv may comprise a heavy chain with one or more CDRs having amino acid sequences set forth in SEQ ID NOs: 47-49. In some embodiments, the CD22-specific scFv may comprise a light chain with one or more CDRs having amino acid sequences set forth in SEQ ID NOs: 51-53. In any of these embodiments, the CD22-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the CD22 CAR comprises or consists of the one or more CDRs as described herein.

In some embodiments, the extracellular binding domain of the CD22 CAR comprises an scFv derived from m971-L7, which is an affinity matured variant of m971 with significantly improved CD22 binding affinity compared to the parental antibody m971 (improved from about 2 nM to less than 50 pM). In some embodiments, the scFv derived from m971-L7 comprises the V_{H} and the V_{L} of m971-L7 connected by a 3×G₄S linker. In other embodiments, the Whitlow linker may be used instead. In some embodiments, the amino acid sequences of the entire m971-L7-derived scFv (also referred to as m971-L7 scFv) and its different portions are provided in Table 15 below. In some embodiments, the CD22-specific scFv comprises or consists of an amino acid sequence set forth in SEQ ID NO:54, 55, or 59, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:54, 55, or 59. In some embodiments, the CD22-specific scFv may comprise one or more CDRs having amino acid sequences set forth in SEQ ID NOs: 56-58 and 60-62. In some embodiments, the CD22-specific scFv may comprise a heavy chain with one or more CDRs having amino acid sequences set forth in SEQ ID NOs: 56-58. In some embodiments, the CD22-specific scFv may comprise a light chain with one or more CDRs having amino acid sequences set forth in SEQ ID NOs: 60-62. In any of these embodiments, the CD22-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the CD22 CAR comprises or consists of the one or more CDRs as described herein.

TABLE 15
Exemplary sequences of anti-CD22 scFv and components (CDRs in bold and underlined)

| SEQ ID NO: | Amino Acid Sequence | Description |
|---|---|---|
| 45 | QVQLQQSGPGLVKPSQTLSLTCAISGDSVSSNSA | Anti-CD22 m971 scFv |
| | AWNWIRQSPSRGLEWLGRTYYRSKWYNDYAV | entire sequence, with |
| | SVKSRITINPDTSKNQFSLQLNSVTPEDTAVYYCA | 3xG4S linker |
| | REVTGDLEDAFDIWGQGTMVTVSSGGGGSGG | |
| | GGSGGGGSDIQMTQSPSSLSASVGDRVTITCRA | |
| | SQTIWSYLNWYQQRPGKAPNLLIYAASSLQSGV | |
| | PSRFSGRGSGTDFTLTISSLQAEDFATYYCQQSYS | |
| | IPQTFGQGTKLEIK | |
| 46 | QVQLQQSGPGLVKPSQTLSLTCAISGDSVSSNSA | Anti-CD22 m971 scFv |
| | AWNWIRQSPSRGLEWLGRTYYRSKWYNDYAV | heavy chain variable |
| | SVKSRITINPDTSKNQFSLQLNSVTPEDTAVYYCA | region |
| | REVTGDLEDAFDIWGQGTMVTVSS | |
| 47 | GDSVSSNSAA | Anti-CD22 m971 scFv |
| | | heavy chain CDR1 |
| 48 | TYYRSKWYN | Anti-CD22 m971 scFv |
| | | heavy chain CDR2 |
| 49 | AREVTGDLEDAFDI | Anti-CD22 m971 scFv |
| | | heavy chain CDR3 |
| 50 | DIQMTQSPSSLSASVGDRVTITCRASQTIWSYLN | Anti-CD22 m971 scFv light |
| | WYQQRPGKAPNLLIYAASSLQSGVPSRFSGRGS | chain |
| | GTDFTLTISSLQAEDFATYYCQQSYSIPQTFGQG | |
| | TKLEIK | |
| 51 | QTIWSY | Anti-CD22 m971 scFv light |
| | | chain CDR1 |
| 52 | AAS | Anti-CD22 m971 scFv light |
| | | chain CDR2 |
| 53 | QQSYSIPQT | Anti-CD22 m971 scFv light |
| | | chain CDR3 |
| 54 | QVQLQQSGPGMVKPSQTLSLTCAISGDSVSSNS | Anti-CD22 m971-L7 scFv |
| | VAWNWIRQSPSRGLEWLGRTYYRSTWYNDYA | entire sequence, with |
| | VSMKSRITINPDTNKNQFSLQLNSVTPEDTAVYY | 3xG₄S linker |
| | CAREVTGDLEDAFDIWGQGTMVTVSSGGGGS | |
| | GGGGSGGGGSDIQMIQSPSSLSASVGDRVTITC | |
| | RASQTIWSYLNWYRQRPGEAPNLLIYAASSLQS | |
| | GVPSRFSGRGSGTDFTLTISSLQAEDFATYYCQQ | |
| | SYSIPQTFGQGTKLEIK | |
| 55 | QVQLQQSGPGMVKPSQTLSLTCAISGDSVSSNS | Anti-CD22 m971-L7 scFv |
| | VAWNWIRQSPSRGLEWLGRTYYRSTWYNDYA | heavy chain variable |
| | VSMKSRITINPDTNKNQFSLQLNSVTPEDTAVYY | region |
| | CAREVTGDLEDAFDIWGQGTMVTVSS | |
| 56 | GDSVSSNSVA | Anti-CD22 m971-L7 scFv |
| | | heavy chain CDR1 |
| 57 | TYYRSTWYN | Anti-CD22 m971-L7 scFv |
| | | heavy chain CDR2 |
| 58 | AREVTGDLEDAFDI | Anti-CD22 m971-L7 scFv |
| | | heavy chain CDR3 |
| 59 | DIQMIQSPSSLSASVGDRVTITCRASQTIWSYLN | Anti-CD22 m971-L7 scFv |
| | WYRQRPGEAPNLLIYAASSLQSGVPSRFSGRGS | light chain variable region |
| | GTDFTLTISSLQAEDFATYYCQQSYSIPQTFGQG | |
| | TKLEIK | |
| 60 | QTIWSY | Anti-CD22 m971-L7 scFv |
| | | light chain CDR1 |
| 61 | AAS | Anti-CD22 m971-L7 scFv |
| | | light chain CDR2 |
| 62 | QQSYSIPQT | Anti-CD22 m971-L7 scFv |
| | | light chain CDR3 |
| 85 | QVQLQQSGPGLVKPSQTLSLTCAISGDSVSSNSA | Anti-CD22 m971 v.2 |
| | AWNWIRQSPSRGLEWLGRTYYRSKWYNDYAV | antigen binding domain |
| | SVKSRITINPDTSKNQFSLQLNSVTPEDTAVYYCA | |
| | REVTGDLEDAFDIWGQGTMVTVSSGGGGSDIQ | |
| | MTQSPSSLSASVGDRVTITCRASQTIWSYLNWY | |
| | QQRPGKAPNLLIYAASSLQSGVPSRFSGRGSGTD | |
| | FTLTISSLQAEDFATYYCQQSYSIPQTFGQGTKLEI | |
| | K | |
| 46 | QVQLQQSGPGLVKPSQTLSLTCAISGDSVSSNSA | Anti-CD22 m971 v.2 |
| | AWNWIRQSPSRGLEWLGRTYYRSKWYNDYAVS | antigen binding domain |
| | VKSRITINPDTSKNQFSLQLNSVTPEDTAVYYCAR | heavy chain variable |
| | EVTGDLEDAFDIWGQGTMVTVSS | region |
| 47 | GDSVSSNSAA | Anti-CD22 m971 v.2 |
| | | antigen binding domain |
| | | heavy chain CDR1 |
| 48 | TYYRSKWYN | Anti-CD22 m971 v.2 |
| | | antigen binding domain |
| | | heavy chain CDR2 |
| 49 | AREVTGDLEDAFDI | Anti-CD22 m971 v.2 |
| | | antigen binding domain |
| | | heavy chain CDR3 |
| 50 | DIQMTQSPSSLSASVGDRVTITCRASQTIWSYLN | Anti-CD22 m971 v.2 |
| | WYQQRPGKAPNLLIYAASSLQSGVPSRFSGRGS | antigen binding domain |
| | GTDFTLTISSLQAEDFATYYCQQSYSIPQTFGQGT | light chain variable region |
| | KLEIK | |
| 51 | QTIWSY | Anti-CD22 m971 v.2 |
| | | antigen binding domain |
| | | light chain CDR1 |
| 52 | AAS | Anti-CD22 m971 v.2 |
| | | antigen binding domain |
| | | light chain CDR2 |
| 53 | QQSYSIPQT | Anti-CD22 m971 v.2 |
| | | antigen binding domain |
| | | light chain CDR3 |
| 86 | TTTPAPRPPTPAPTIASQPLSLRPEACRPAAGGA | CD8 transmembrane |
| | VHTRGLDFACDIYIWAPLAGTCGVLLLSLVITLYC | domain |
| 87 | VMYPPPYLDNEKSNGTIIHVKGKHLCPSPLFPGP | CD28 transmembrane |
| | SKPFWVLVVVGGVLACYSLLVTVAFIIFWVR | domain |
| 88 | SKRSRLLHSDYMNMTPRRPGPTRKHYQPYAPPR | CD28 costimulatory |
| | DFAAYRS | domain |
| 89 | FVPVFLPAKPTTTPAPRPPTPAPTIASQPLSLRPE | CD8 costimulatory domain |
| | ACRPAAGGAVHTRGLDFACDIYIWAPLAGTCGV | |
| | LLLSLVITLYCNHRNR | |
| 90 | RFSVVKRGRKKLLYIFKQPFMRPVQTTQEEDGCS | CD137 costimulatory |
| | CRFPEEEEGGCEL | domain |
| 91 | MLLLVTSLLLCELPHPAFLLIPQVQLQQSGPGLVK | anti-CD22 CAR v.1 with |
| | PSQTLSLTCAISGDSVSSNSAAWNWIRQSPSRG | m971 antigen binding |
| | LEWLGRTYYRSKWYNDYAVSVKSRITINPDTSK | domain, CD8 |
| | NQFSLQLNSVTPEDTAVYYCAREVTGDLEDAFDI | transmembrane domain, |
| | WGQGTMVTVSSGGGGSDIQMTQSPSSLSASV | and CD137 and CD3ζ, |
| | GDRVTITCRASQTIWSYLNWYQQRPGKAPNLLI | intracellular T cell |
| | YAASSLQSGVPSRFSGRGSGTDFTLTISSLQAEDF | signaling domains |
| | ATYYCQQSYSIPQTFGQGTKLEIKAAATTTPAPR | |
| | PPTPAPTIASQPLSLRPEACRPAAGGAVHTRGLD | |
| | FACDIYIWAPLAGTCGVLLLSLVITLYCKRGRKKLL | |
| | YIFKQPFMRPVQTTQEEDGCSCRFPEEEEGGCEL | |
| | RVKFSRSADAPAYKQGQNQLYNELNLGRREEYD | |
| | VLDKRRGRDPEMGGKPRRKNPQEGLYNELQKD | |
| | KMAEAYSEIGMKGERRRGKGHDGLYQGLSTAT | |
| | KDTYDALHMQALPPR | |
| 85 | QVQLQQSGPGLVKPSQTLSLTCAISGDSVSSNSA | anti-CD22 CAR v.1 antigen |
| | AWNWIRQSPSRGLEWLGRTYYRSKWYNDYAVS | binding domain |
| | VKSRITINPDTSKNQFSLQLNSVTPEDTAVYYCAR | |
| | EVTGDLEDAFDIWGQGTMVTVSSGGGGSDIQ | |
| | MTQSPSSLSASVGDRVTITCRASQTIWSYLNWY | |
| | QQRPGKAPNLLIYAASSLQSGVPSRFSGRGSGTD | |
| | FTLTISSLQAEDFATYYCQQSYSIPQTFGQGTKLEI | |
| | K | |
| 46 | QVQLQQSGPGLVKPSQTLSLTCAISGDSVSSNSA | anti-CD22 CAR v.1 antigen |
| | AWNWIRQSPSRGLEWLGRTYYRSKWYNDYAVS | binding domain heavy |
| | VKSRITINPDTSKNQFSLQLNSVTPEDTAVYYCAR | chain variable region |
| | EVTGDLEDAFDIWGQGTMVTVSS | |
| 47 | GDSVSSNSAA | anti-CD22 CAR v.1 antigen |
| | | binding domain heavy |
| | | chain CDR1 |
| 48 | TYYRSKWYN | anti-CD22 CAR v.1 antigen |
| | | binding domain heavy |
| | | chain CDR2 |
| 49 | AREVTGDLEDAFDI | anti-CD22 CAR v.1 antigen |
| | | binding domain heavy |
| | | chain CDR3 |
| 50 | DIQMTQSPSSLSASVGDRVTITCRASQTIWSYLN | anti-CD22 CAR v.1 antigen |
| | WYQQRPGKAPNLLIYAASSLQSGVPSRFSGRGS | binding domain light chain |
| | GTDFTLTISSLQAEDFATYYCQQSYSIPQTFGQGT | variable region |
| | KLEIK | |
| 51 | QTIWSY | anti-CD22 CAR v.1 antigen |
| | | binding domain light chain |
| | | CDR1 |
| 52 | AAS | anti-CD22 CAR v.1 antigen |
| | | binding domain light chain |
| | | CDR2 |
| 53 | QQSYSIPQT | anti-CD22 CAR v.1 antigen |
| | | binding domain light chain |
| | | CDR3 |
| 92 | MLLLVTSLLLCELPHPAFLLIPQVQLQQSGPGLVK | anti-CD22 CAR v.2 with |
| | PSQTLSLTCAISGDSVSSNSAAWNWIRQSPSRG | m971 antigen binding |
| | LEWLGRTYYRSKWYNDYAVSVKSRITINPDTSK | domain, CD28 |
| | NQFSLQLNSVTPEDTAVYYCAREVTGDLEDAFDI | transmembrane domain, |
| | WGQGTMVTVSSGGGGSDIQMTQSPSSLSASV | and CD28 and CD3ζ |
| | GDRVTITCRASQTIWSYLNWYQQRPGKAPNLLI | intracellular T cell |
| | YAASSLQSGVPSRFSGRGSGTDFTLTISSLQAEDF | signaling domains |
| | ATYYCQQSYSIPQTFGQGTKLEIKAAAIEVMYPP | |
| | PYLDNEKSNGTIIHVKGKHLCPSPLFPGPSKPFW | |
| | VLVVVGGVLACYSLLVTVAFIIFWVRSKRSRLLHS | |
| | DYMNMTPRRPGPTRKHYQPYAPPRDFAAYRSR | |
| | VKFSRSADAPAYQQGQNQLYNELNLGRREEYDV | |
| | LDKRRGRDPEMGGKPRRKNPQEGLYNELQKDK | |
| | MAEAYSEIGMKGERRRGKGHDGLYQGLSTATK | |
| | DTYDALHMQALPPR | |
| 85 | QVQLQQSGPGLVKPSQTLSLTCAISGDSVSSNSA | anti-CD22 CAR v.2 antigen |
| | AWNWIRQSPSRGLEWLGRTYYRSKWYNDYAVS | binding domain |
| | VKSRITINPDTSKNQFSLQLNSVTPEDTAVYYCAR | |
| | EVTGDLEDAFDIWGQGTMVTVSSGGGGSDIQ | |
| | MTQSPSSLSASVGDRVTITCRASQTIWSYLNWY | |
| | QQRPGKAPNLLIYAASSLQSGVPSRFSGRGSGTD | |
| | FTLTISSLQAEDFATYYCQQSYSIPQTFGQGTKLEI | |
| | K | |
| 46 | QVQLQQSGPGLVKPSQTLSLTCAISGDSVSSNSA | anti-CD22 CAR v.2 antigen |
| | AWNWIRQSPSRGLEWLGRTYYRSKWYNDYAVS | binding domain heavy |
| | VKSRITINPDTSKNQFSLQLNSVTPEDTAVYYCAR | chain variable region |
| | EVTGDLEDAFDIWGQGTMVTVSS | |
| 47 | GDSVSSNSAA | anti-CD22 CAR v.2 antigen |
| | | binding domain heavy |
| | | chain CDR1 |
| 48 | TYYRSKWYN | anti-CD22 CAR v.2 antigen |
| | | binding domain heavy |
| | | chain CDR2 |
| 49 | AREVTGDLEDAFDI | anti-CD22 CAR v.2 antigen |
| | | binding domain heavy |
| | | chain CDR3 |
| 50 | DIQMTQSPSSLSASVGDRVTITCRASQTIWSYLN | anti-CD22 CAR v.2 antigen |
| | WYQQRPGKAPNLLIYAASSLQSGVPSRFSGRGS | binding domain light chain |
| | GTDFTLTISSLQAEDFATYYCQQSYSIPQTFGQGT | variable region |
| | KLEIK | |
| 51 | QTIWSY | anti-CD22 CAR v.2 antigen |
| | | binding domain light chain |
| | | CDR1 |
| 52 | AAS | anti-CD22 CAR v.2 antigen |
| | | binding domain light chain |
| | | CDR2 |
| 53 | QQSYSIPQT | anti-CD22 CAR v.2 antigen |
| | | binding domain light chain |
| | | CDR3 |
| 93 | MLLLVTSLLLCELPHPAFLLIPQVQLQQSGPGLVK | anti-CD22 CAR v.3 with |
| | PSQTLSLTCAISGDSVSSNSAAWNWIRQSPSRG | m971 antigen binding |
| | LEWLGRTYYRSKWYNDYAVSVKSRITINPDTSK | domain, CD8 |
| | NQFSLQLNSVTPEDTAVYYCAREVTGDLEDAFDI | transmembrane domain, |
| | WGQGTMVTVSSGGGGSDIQMTQSPSSLSASV | and CD28, CD137, and |
| | GDRVTITCRASQTIWSYLNWYQQRPGKAPNLLI | CD3ζ intracellular T cell |
| | YAASSLQSGVPSRFSGRGSGTDFTLTISSLQAEDF | signaling domains |
| | ATYYCQQSYSIPQTFGQGTKLEIKAAAFVPVFLP | |
| | AKPTTTPAPRPPTPAPTIASQPLSLRPEACRPAAG | |
| | GAVHTRGLDFACDIYIWAPLAGTCGVLLLSLVITL | |
| | YCNHRNRSKRSRLLHSDYMNMTPRRPGPTRKH | |
| | YQPYAPPRDFAAYRSRFSVVKRGRKKLLYIFKQPF | |
| | MRPVQTTQEEDGCSCRFPEEEEGGCELRVKFSR | |
| | SADAPAYQQGQNQLYNELNLGRREEYDVLDKR | |
| | RGRDPEMGGKPRRKNPQEGLYNELQKDKMAE | |
| | AYSEIGMKGERRRGKGHDGLYQGLSTATKDTYD | |
| | ALHMQALPPR | |
| 85 | QVQLQQSGPGLVKPSQTLSLTCAISGDSVSSNSA | anti-CD22 CAR v.3 antigen |
| | AWNWIRQSPSRGLEWLGRTYYRSKWYNDYAVS | binding domain |
| | VKSRITINPDTSKNQFSLQLNSVTPEDTAVYYCAR | |
| | EVTGDLEDAFDIWGQGTMVTVSSGGGGSDIQ | |
| | MTQSPSSLSASVGDRVTITCRASQTIWSYLNWY | |
| | QQRPGKAPNLLIYAASSLQSGVPSRFSGRGSGTD | |
| | FTLTISSLQAEDFATYYCQQSYSIPQTFGQGTKLEI | |
| | K | |
| 46 | QVQLQQSGPGLVKPSQTLSLTCAISGDSVSSNSA | anti-CD22 CAR v.3 antigen |
| | AWNWIRQSPSRGLEWLGRTYYRSKWYNDYAVS | binding domain heavy |
| | VKSRITINPDTSKNQFSLQLNSVTPEDTAVYYCAR | chain variable region |
| | EVTGDLEDAFDIWGQGTMVTVSS | |
| 47 | GDSVSSNSAA | anti-CD22 CAR v.3 antigen |
| | | binding domain heavy |
| | | chain CDR1 |
| 48 | TYYRSKWYN | anti-CD22 CAR v.3 antigen |
| | | binding domain heavy |
| | | chain CDR2 |
| 49 | AREVTGDLEDAFDI | anti-CD22 CAR v.3 antigen |
| | | binding domain heavy |
| | | chain CDR3 |
| 50 | DIQMTQSPSSLSASVGDRVTITCRASQTIWSYLN | anti-CD22 CAR v.3 antigen |
| | WYQQRPGKAPNLLIYAASSLQSGVPSRFSGRGS | binding domain light chain |
| | GTDFTLTISSLQAEDFATYYCQQSYSIPQTFGQGT | variable region |
| | KLEIK | |
| 51 | QTIWSY | anti-CD22 CAR v.3 antigen |
| | | binding domain light chain |
| | | CDR1 |
| 52 | AAS | anti-CD22 CAR v.3 antigen |
| | | binding domain light chain |
| | | CDR2 |
| 53 | QQSYSIPQT | anti-CD22 CAR v.3 antigen |
| | | binding domain light chain |
| | | CDR3 |

In some embodiments, the extracellular binding domain of the CD22 CAR comprises immunotoxins HA22 or BL22. Immunotoxins BL22 and HA22 are therapeutic agents that comprise an scFv specific for CD22 fused to a bacterial toxin, and thus can bind to the surface of the cancer cells that express CD22 and kill the cancer cells. BL22 comprises a dsFv of an anti-CD22 antibody, RFB4, fused to a 38-kDa truncated form of Pseudomonas exotoxin A (Bang et al., Clin. Cancer Res., 11:1545-50 (2005)). HA22 (CAT8015, moxetumomab pasudotox) is a mutated, higher affinity version of BL22 (Ho et al., J. Biol. Chem., 280(1): 607-17 (2005)). Suitable sequences of antigen binding domains of HA22 and BL22 specific to CD22 are disclosed in, for example, U.S. Pat. Nos. 7,541,034; 7,355,012; and 7,982,011, which are hereby incorporated by reference in their entirety.

In some embodiments, the hinge domain of the CD22 CAR comprises a CD8a hinge domain, for example, a human CD8a hinge domain. In some embodiments, the CD8a hinge domain comprises or consists of an amino acid sequence set forth in SEQ ID NO:9 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:9. In some embodiments, the hinge domain comprises a CD28 hinge domain, for example, a human CD28 hinge domain. In some embodiments, the CD28 hinge domain comprises or consists of an amino acid sequence set forth in SEQ ID NO:10 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:10. In some embodiments, the hinge domain comprises an IgG4 hinge domain, for example, a human IgG4 hinge domain. In some embodiments, the IgG4 hinge domain comprises or consists of an amino acid sequence set forth in SEQ ID NO:11 or SEQ ID NO:12, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:11 or SEQ ID NO:12. In some embodiments, the hinge domain comprises a IgG4 hinge-Ch2-Ch3 domain, for example, a human IgG4 hinge-Ch2-Ch3 domain. In some embodiments, the IgG4 hinge-Ch2-Ch3 domain comprises or consists of an amino acid sequence set forth in SEQ ID NO:13 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:13.

In some embodiments, the transmembrane domain of the CD22 CAR comprises a CD8a transmembrane domain, for example, a human CD8a transmembrane domain. In some embodiments, the CD8a transmembrane domain comprises or consists of an amino acid sequence set forth in SEQ ID NO:14 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:14. In some embodiments, the transmembrane domain comprises a CD28 transmembrane domain, for example, a human CD28 transmembrane domain. In some embodiments, the CD28 transmembrane domain comprises or consists of an amino acid sequence set forth in SEQ ID NO:15 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:15.

In some embodiments, the intracellular costimulatory domain of the CD22 CAR comprises a 4-1BB costimulatory domain, for example, a human 4-1BB costimulatory domain. In some embodiments, the 4-1BB costimulatory domain comprises or consists of an amino acid sequence set forth in SEQ ID NO:16 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:16. In some embodiments, the intracellular costimulatory domain comprises a CD28 costimulatory domain, for example, a human CD28 costimulatory domain. In some embodiments, the CD28 costimulatory domain comprises or consists of an amino acid sequence set forth in SEQ ID NO:17 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:17.

In some embodiments, the intracellular signaling domain of the CD22 CAR comprises a CD3 zeta (ζ) signaling domain, for example, a human CD3ζ signaling domain. In some embodiments, the CD3ζ signaling domain comprises or consists of an amino acid sequence set forth in SEQ ID NO:18 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:18.

In some embodiments, the polycistronic vector comprises an expression cassette that contains a nucleotide sequence encoding a CD22 CAR, including, for example, a CD22 CAR comprising the CD22-specific scFv having sequences set forth in SEQ ID NO:45 or SEQ ID NO:54, the CD8a hinge domain of SEQ ID NO:9, the CD8a transmembrane domain of SEQ ID NO:14, the 4-1BB costimulatory domain of SEQ ID NO:16, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD22 CAR, including, for example, a CD22 CAR comprising the CD22-specific scFv having sequences set forth in SEQ ID NO:45 or SEQ ID NO:54, the CD28 hinge domain of SEQ ID NO:10, the CD8a transmembrane domain of SEQ ID NO:14, the 4-1BB costimulatory domain of SEQ ID NO:16, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD22 CAR, including, for example, a CD22 CAR comprising the CD22-specific scFv having sequences set forth in SEQ ID NO:45 or SEQ ID NO:54, the IgG4 hinge domain of SEQ ID NO:11 134 or SEQ ID NO:12, the CD8a transmembrane domain of SEQ ID NO:14, the 4-1BB costimulatory domain of SEQ ID NO:16, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD22 CAR, including, for example, a CD22 CAR comprising the CD22-specific scFv having sequences set forth in SEQ ID NO:45 or SEQ ID NO:54, the CD8a hinge domain of SEQ ID NO:9, the CD28 transmembrane domain of SEQ ID NO:15, the 4-1BB costimulatory domain of SEQ ID NO:16, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD22 CAR, including, for example, a CD22 CAR comprising the CD22-specific scFv having sequences set forth in SEQ ID NO:45 or SEQ ID NO:54, the CD28 hinge domain of SEQ ID NO:10, the CD28 transmembrane domain of SEQ ID NO:15, the 4-1BB costimulatory domain of SEQ ID NO:16, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD22 CAR, including, for example, a CD22 CAR comprising the CD22-specific scFv having sequences set forth in SEQ ID NO:45 or SEQ ID NO:54, the IgG4 hinge domain of SEQ ID NO:11 or SEQ ID NO:12, the CD28 transmembrane domain of SEQ ID NO:15, the 4-1BB costimulatory domain of SEQ ID NO:16, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the CAR comprises a transmembrane domain comprising CD28 and an intracellular signaling domain comprising CD28 and CD3ζ signaling domains.

In some embodiments, the CAR comprises a transmembrane domain comprising CD8 and an intracellular signaling domain comprising CD28, CD137, and CD3ζ signaling domains.

In some embodiments, the CAR comprises a transmembrane domain comprising CD8 and an intracellular signaling domain comprising CD137 and CD3ζ signaling domains.

In some embodiments, the CAR has a sequence at least 80% identical, at least 85% identical, at least 90% identical, at least 95% identical, at least 96% identical, at least 97% identical, at least 98% identical, at least 99% identical, or 100% identical to the amino acid sequence of SEQ ID NO: 91. In some embodiments, the CAR having an amino acid sequence of SEQ ID NO: 91 is a second generation CAR.

In some embodiments, the CAR has a sequence at least 80% identical, at least 85% identical, at least 90% identical, at least 95% identical, at least 96% identical, at least 97% identical, at least 98% identical, at least 99% identical, or 100% identical to the amino acid sequence of SEQ ID NO: 92. In some embodiments, the CAR having an amino acid sequence of SEQ ID NO: 92 is a second generation CAR.

In some embodiments, the CAR has a sequence at least 80% identical, at least 85% identical, at least 90% identical, at least 95% identical, at least 96% identical, at least 97% identical, at least 98% identical, at least 99% identical, or 100% identical to the amino acid sequence of SEQ ID NO: 93. In some embodiments, the CAR having an amino acid sequence of SEQ ID NO: 93 is a third generation CAR.

In some embodiments, a polynucleotide provided herein comprises a nucleotide sequence encoding a CD22 CAR, a variable domain of a CD22 CAR, or a set of CDRs (HCDR 1, 2, and 3 and LCDR 1, 2, and 3) that encodes a CD22 CAR as set forth in TABLE 16 below or a variable domain of a CD22 CAR or a set of CDRs (HCDR 1, 2, and 3 and LCDR 1, 2, and 3) therefrom. In some embodiments, a polynucleotide provided herein comprises a nucleotide sequence encoding a CD22 CAR, a variable domain of a CD22 CAR, or a set of CDRs (HCDR 1, 2, and 3 and LCDR 1, 2, and 3) that having at least 80% (e.g., at least 80%, at least 85%, at least 90%, at least 95%, at least 96%, at least 97%, at least 98%, at least 99%, or 100% identical) to a CD22 CAR as set forth in TABLE 16 below or a variable domain of a CD22 CAR or a set of CDRs (HCDR 1, 2, and 3 and LCDR 1, 2, and 3) therefrom, respectively.

TABLE 16
Exemplary CD22 antigen binding domains

| Antibody Name | Patents | Publications | Company |
|---|---|---|---|
| ADC Therapeutics | 2020 US20200306385 | AACR 2015 Preclinical activity of hLL2- | ADC Therapeutics |
| patent anti-CD22 | 2018 US20180326062 | PBD, a novel anti-CD22 antibody- | Medimmune |
| | 2018 US20180169258 | pyrrolobenzodiazepine (PBD) | |
| | 2018 U.S. Patent No. 10,722,594 | conjugate in models of non-Hodgkin | |
| | 2016 WO2017059289 | lymphoma | |
| | 2014 WO2015052535 | | |
| | 2014 US20160250346 | | |
| | 2014 U.S. Patent No. 9,956,299 | | |
| | 2013 WO2014057118 | | |
| | 2013 WO2014057122 | | |
| | 2013 US20150265722 | | |
| | 2013 U.S. Patent No. 9,919,056 | | |
| ADCT-602 | 2018 US20200405879 | 2018 NCT03698552 Phase 1/Phase 2 | ADC Therapeutics |
| | | ADCT-602 in Treating Patients With | |
| | | Recurrent or Refractory B-cell Acute | |
| | | Lymphoblastic Leukemia | |
| BAY1862864 | | 2015 NCT02581878 Phase 1 Safety | Bayer |
| | | and Tolerability of BAY1862864 | |
| | | Injection in Subjects With Relapsed or | |
| | | Refractory CD22-positive Non- | |
| | | Hodgkin's Lymphoma | |
| | | 2021 Lindén O, Bates A . . . Thorium- | |
| | | 227-Labeled Anti-CD22 Antibody (BAY | |
| | | 1862864) in Relapsed/Refractory | |
| | | CD22-Positive Non-Hodgkin | |
| | | Lymphoma: A First-in-Human, Phase I | |
| | | Study. | |
| bectumomab | 2011 US20110305631 | | Immunomedics |
| | 2011 U.S. Patent No. 8,420,086 | | |
| Bioalliance patent | 2015 WO2015196089 | | AltruBio Bioalliance |
| anti-CD22 | 2015 US20160015831 | | |
| Bioatla patent anti- | 2017 US20180086843 | 2016 Guzel H, Bakbak B . . . The effect | BioAtla Femta |
| CD22 | 2013 WO2013163519 | and safety of intravitreal injection of | |
| | 2013 US20150086562 | ranibizumab and bevacizumab on the | |
| | 2013 U.S. Patent No. 9,856,323 | corneal endothelium in the treatment | |
| | | of diabetic macular edema. | |
| Cellectis patent | 2018 WO2018178378 | | Cellectis |
| anti-CD22 CAR | 2018 WO2018178377 | | |
| Children's | 2020 WO2021021846 | | Children's |
| Hosp. Philadelphia | 2020 US20220289841 | | Hosp.Philadelphia |
| patent anti-CD22 | | | |
| Chugai patent anti- | 2004 WO2004087763 | | Chugai |
| CD22 | | | |
| Datamabs patent | 2013 US20130266558 | | Datamabs |
| anti-CD22 | | | |
| Duke patent anti- | 2019 US20190367607 | | Duke U. |
| CD22 | TEDDER, Thomas F . . . | | |
| | ANTIBODIES AND | | |
| | METHODS FOR | | |
| | DEPLETING | | |
| | REGULATORY B10 CELLS | | |
| | AND USE IN | | |
| | COMBINATION WITH | | |
| | IMMUNE CHECKPOINT | | |
| | INHIBITORS | | |
| | 2017 WO2018112407 | | |
| | 2012 US20120301472 | | |
| | 2012 U.S. Patent No. 8,734,792 | | |
| | 2007 US20080118505 | | |
| | 2003 WO2003072036 | | |
| Elpis Bio patent | 2020 WO2021035174 | | Elpis Bio |
| anti-CD22 | 2020 US20220298257 | | |
| epratuzumab | 2020 US20200157215 | 2016 NCT02577094 Phase 1/Phase 2 | Amgen |
| | 2020 U.S. Patent No. 11,472,879 | Testing a Reduced Conditioning | Immunomedics UCB |
| | 2017 US20170226205 | Regimen FB2A2 Preceded by a | |
| | 2017 U.S. Pat. No. 9,963,507 | Fractionated Radio-immunotherapy | |
| | 2017 US20170151356 | (RIT) With 90Y-Epratuzumab Before | |
| | 2016 US20160368986 | Allogeneic Stem Cell Transplantation | |
| | 2016 U.S. Pat. No. 9,663,576 | for Patients With Lymphocyte B CD22 | |
| | 2016 US20180273620 | Positive Acute Lymphoblastic | |
| | 2016 U.S. Pat. No. 10,590,197 | Leukemia | |
| | 2016 US20160251444 | 2014 NCT02306629 Phase 1 Study to | |
| | 2016 US20160304611 | Compare Properties of Epratuzumab | |
| | 2016 US20160296648 | When Given as an Injection Under the | |
| | 2016 WO2016164264 | Skin or Directly Into the Blood | |
| | 2015 US20160032003 | 2012 NCT01534403 Phase 2 Open | |
| | 2015 U.S. Pat. No. 9,475,883 | Label Extension Study of Epratuzumab | |
| | 2014 US20150023870 | in Japanese Systemic Lupus | |
| | 2014 U.S. Pat. No. 9,737,617 | Erythematosus (SLE) Subjects | |
| | 2014 US20140369927 | 2011 NCT01449071 Phase 1/Phase 2 | |
| | 2014 US20140212425 | Study Evaluating the | |
| | 2014 US20140147382 | Pharmacokinetics and Safety of | |
| | 2013 U.S. Pat. No. 9,102,735 | Epratuzumab in Japanese Systemic | |
| | 2013 US20150150979 | Lupus Erythematosus (SLE) | |
| | 2013 US20130177526 | 2011 NCT01408576 Phase 3 Open | |
| | 2012 US20130142787 | Label Extension Study of Epratuzumab | |
| | 2012 WO2013085893 | in Subjects With Systemic Lupus | |
| | 2012 U.S. Pat. No. 9,192,664 | Erythematosus (EMBODY4) | |
| | 2012 US20130078183 | 2010 NCT01261793 Phase 3 Study of | |
| | 2012 U.S. Pat. No. 8,871,216 | Epratuzumab Versus Placebo in | |
| | 2012 US20120302739 | Subjects With Moderate to Severe | |
| | 2012 US20130171170 | General Systemic Lupus | |
| | 2011 US20120183472 | Erythematosus (SLE) | |
| | 2011 US20110256053 | 2010 NCT01262365 Phase 3 Study of | |
| | 2011 US20110165073 | Epratuzumab Versus Placebo in | |
| | 2011 U.S. Pat. No. 8,105,596 | Subjects With Moderate to Severe | |
| | 2010 WO2011032633 | General Systemic Lupus | |
| | 2008 U.S. Pat. No. 7,919,606 | Erythematosus | |
| | 2008 US20100021995 | 2010 NCT01219816 Phase 2 Multi- | |
| | 2007 US20070172920 | centric Study (CHEPRALL) | |
| | 2006 U.S. Pat. No. 7,527,787 | 2010 NCT00945815 Phase 2 | |
| | 2005 U.S. Pat. No. 7,939,073 | Epratuzumab, Cytarabine, and | |
| | 2005 U.S. Pat. No. 7,811,570 | Clofarabine in Treating Patients With | |
| | 2004 US20050106108 | Relapsed or Refractory Acute | |
| | 2003 US20040013607 | Lymphoblastic Leukemia | |
| | 2003 WO2003093320 | 2010 NCT01147393 Phase 1/Phase 2 | |
| | 2002 U.S. Pat. No. 7,837,995 | Combination Veltuzumab and | |
| | 2001 U.S. Pat. No. 7,910,103 | Fractionated 90Y- Epratuzumab | |
| | 2001 WO2001097858 | Radioimmunotherapy in Follicular | |
| | 2000 US20020102254 | Lymphoma | |
| | 1998 U.S. Pat. No. 6,187,287 | 2010 NCT01101581 Phase 1/Phase 2 | |
| | 1996 U.S. Pat. No. 5,789,554 | Study of Veltuzumab Combined With | |
| | 1995 WO1996004925 | 90Y-Epratuzumab Tetraxetan in | |
| | | Patients With Relapsed/Refractory, | |
| | | Aggressive Non-Hodgkin's Lymphoma | |
| | | 2010 NCT01085617 Phase 3 Standard | |
| | | Chemotherapy With or Without | |
| | | Nelarabine or Epratuzumab and/or | |
| | | Rituximab in Treating Patients With | |
| | | Newly Diagnosed Acute | |
| | | Lymphoblastic Leukemia | |
| | | 2010 NCT01279707 Phase 1/Phase 2 | |
| | | Monoclonal Antibodies in Recurrent B | |
| | | Cell Acute Lymphoblastic Leukaemia | |
| | | (ALL) (MARALL) | |
| | | 2009 NCT00941928 Phase 2 | |
| | | Haploidentical Natural Killer (NK) Cells | |
| | | With Epratuzumab for Relapsed Acute | |
| | | Lymphoblastic Leukemia (ALL) | |
| | | 2008 NCT00660881 Phase 2 Open- | |
| | | label Study of Epratuzumab in | |
| | | Serologically-positive Systemic Lupus | |
| | | Erythematosus Patients With Active | |
| | | Disease | |
| | | 2008 NCT00553501 Phase 2 | |
| | | Epratuzumab and Rituximab in | |
| | | Treating Patients With Previously | |
| | | Untreated Follicular Non-Hodgkin | |
| | | Lymphoma | |
| | | 2008 NCT00624351 Phase 2 Study of | |
| | | Epratuzumab in Serologically-positive | |
| | | Systemic Lupus Erythematosus | |
| | | Patients With Active Disease | |
| | | 2007 NCT00504972 Phase 1 Phase I | |
| | | Trial of Anti-CD74 (hLL1) Antibody | |
| | | Therapy in B Cell Malignancies | |
| | | 2006 NCT00383513 Phase 3 Study of | |
| | | Epratuzumab in Systemic Lupus | |
| | | Erythematosus | |
| | | 2006 NCT00301821 Phase 2 | |
| | | Monoclonal Antibody Therapy and | |
| | | Combination Chemotherapy in | |
| | | Treating Patients With Stage II, Stage | |
| | | III, or Stage IV Diffuse Large B-Cell | |
| | | Lymphoma | |
| | | 2005 NCT00111306 Phase 3 Study of | |
| | | Epratuzumab in Systemic Lupus | |
| | | Erythematosus | |
| | | 2005 NCT00098839 Phase 2 | |
| | | Epratuzumab and Combination | |
| | | Chemotherapy in Treating Young | |
| | | Patients With Relapsed Acute | |
| | | Lymphoblastic Leukemia | |
| | | 2004 NCT00113802 Phase 2 Study of | |
| | | Epratuzumab (hLL2) in Patients With | |
| | | Waldenstrom's Macroglobulinemia | |
| | | 2002 NCT00421395 Phase 1/Phase 2 | |
| | | Safety Study of NHL With 90Y-hLL2 | |
| | | IgG | |
| | | 2002 NCT00042913 Phase 2 | |
| | | Epratuzumab in Treating Patients | |
| | | With Non-Hodgkin's Lymphoma | |
| | | 2001 NCT00022685 Phase 3 | |
| | | Epratuzumab in Treating Patients | |
| | | With Non-Hodgkin's Lymphoma | |
| | | 2001 NCT00011908 Phase 1 | |
| | | Humanized LL2IGG to Treat Systemic | |
| | | Lupus Erythematosus | |
| | | 2000 NCT00398372 N/A Clinical and | |
| | | Pathologic Studies in Non-Hodgkin's | |
| | | Lymphoma Patients Receiving | |
| | | Antibody Treatment | |
| | | 2000 NCT00061425 Phase 1/Phase 2 | |
| | | Treatment of Non-Hodgkin's | |
| | | Lymphoma With 90Y-hLL2 lgG | |
| | | 1998 NCT00004084 Phase 1/Phase 2 | |
| | | Radiolabeled Monoclonal Antibody | |
| | | Therapy in Treating Patients With | |
| | | Lymphoma or Leukemia | |
| | | 1998 NCT00004107 Phase 1/Phase 2 | |
| | | Radiolabeled Monoclonal Antibody | |
| | | Therapy Plus Peripheral Stem Cell | |
| | | Transplantation in Treating Patients | |
| | | With Lymphoma or Waldenstrom's | |
| | | Macroglobulinemia | |
| | | 1997 NCT00004086 Phase 1 | |
| | | Radiolabeled Monoclonal Antibody, | |
| | | Combination Chemotherapy, and | |
| | | Peripheral Stem Cell Transplantation | |
| | | in Treating Patients With Recurrent or | |
| | | Refractory B-Cell Cancer | |
| | | 1997 NCT00003337 Phase 3 | |
| | | Radiolabeled Monoclonal Antibody in | |
| | | the Detection and Staging of Patients | |
| | | With Non-Hodgkin's Lymphoma | |
| | | 1997 NCT00003338 Phase 2/Phase 3 | |
| | | Monoclonal Antibodies in Detecting | |
| | | Residual Disease in Patients Who | |
| | | Have Been Treated for Non-Hodgkin's | |
| | | Lymphoma | |
| | | NCT00044902 Phase 2 Monoclonal | |
| | | Antibody Treatment for Non- | |
| | | Hodgkin's Lymphoma (Low-Grade) | |
| | | 2022 McClane J, Chawla . . . Direct CNS | |
| | | administration of rituximab and | |
| | | epratuzumab in a pediatric patient | |
| | | with relapsed refractory CNS B-cell | |
| | | acute lymphoblastic leukemia. | |
| | | 2019 Li J, Wei M M, Son . . . Anti-CD22 | |
| | | epratuzumab for systemic lupus | |
| | | erythematosus: A systematic review | |
| | | and meta-analysis of randomized | |
| | | controlled trials. | |
| | | 2018 Geh D, Gordon C Epratuzumab | |
| | | for the treatment of systemic lupus | |
| | | erythematosus. | |
| | | 2018 Gottenberg J E, Dö . . . Efficacy of | |
| | | Epratuzumab, an Anti-CD22 | |
| | | Monoclonal IgG Antibody, in Systemic | |
| | | Lupus Erythematosus Patients with | |
| | | Associated Sjögren's Syndrome: Post- | |
| | | hoc Analyses from the EMBODY Trials. | |
| | | 2017 Ereño-Orbea J, Si . . . Structural | |
| | | Basis of Enhanced Crystallizability | |
| | | Induced by a Molecular Chaperone | |
| | | for Antibody Antigen-Binding | |
| | | Fragments. | |
| | | 2017 Ereño-Orbea J, Si . . . Molecular | |
| | | basis of human CD22 function and | |
| | | therapeutic targeting. | |
| | | 2017 Valecha G K, Ibrah . . . Emerging | |
| | | Role of Immunotherapy in Precursor | |
| | | B-cell Lymphoblastic Leukemia. | |
| | | 2016 Clowse M E, Wallac . . . Efficacy | |
| | | and Safety of Epratuzumab in | |
| | | Moderately to Severely Active | |
| | | Systemic Lupus Erythematosus: | |
| | | Results from the Phase 3, | |
| | | Randomized, Double-blind, Placebo- | |
| | | controlled Trials, EMBODY ™ 1 and | |
| | | EMBODY ™ 2. | |
| | | 2016 Özgör L, Brandl C . . . | |
| | | Epratuzumab modulates B-cell | |
| | | signalling without affecting B-cell | |
| | | numbers or B- cell functions in a | |
| | | mouse model with humanised CD22. | |
| | | 2016 Lumb S, Fleischer . . . Engagement | |
| | | of CD22 on B cells with the | |
| | | monoclonal antibody epratuzumab | |
| | | stimulates the phosphorylation of | |
| | | upstream inhibitory signals of the B | |
| | | cell receptor. | |
| | | 2015 Dörner T, Shock A . . . The | |
| | | mechanistic impact of CD22 | |
| | | engagement with epratuzumab on B | |
| | | cell function: Implications for the | |
| | | treatment of systemic lupus | |
| | | erythematosus. | |
| | | 2015 Raetz E A, Cairo M . . . Re- | |
| | | induction chemoimmunotherapy with | |
| | | epratuzumab in relapsed acute | |
| | | lymphoblastic leukemia (ALL): Phase II | |
| | | results from Children's Oncology | |
| | | Group (COG) study ADVL04P2. | |
| | | 2014 Antoniu S Epratuzumab for | |
| | | systemic lupus erythematosus. | |
| | | 2013 Strand V, Petri M . . . | |
| | | Epratuzumab for patients with | |
| | | moderate to severe flaring SLE: | |
| | | health-related quality of life | |
| | | outcomes and corticosteroid use in | |
| | | the randomized controlled ALLEVIATE | |
| | | trials and extension study SL0006. | |
| | | 2013 Grant B W, Jung S H . . . A phase 2 | |
| | | trial of extended induction | |
| | | epratuzumab and rituximab for | |
| | | previously untreated follicular | |
| | | lymphoma: CALGB 50701. | |
| | | 2013 Rossi E A, Goldenb . . . | |
| | | Trogocytosis of multiple B-cell surface | |
| | | markers by CD22 targeting with | |
| | | epratuzumab. | |
| | | 2013 Wallace D J, Gordo . . . Efficacy and | |
| | | safety of epratuzumab in patients | |
| | | with moderate/severe flaring | |
| | | systemic lupus erythematosus: results | |
| | | from two randomized, double-blind, | |
| | | placebo-controlled, multicentre | |
| | | studies (ALLEVIATE) and follow-up. | |
| | | 2013 Wallace D J, Kalun . . . Efficacy and | |
| | | safety of epratuzumab in patients | |
| | | with moderate/severe active systemic | |
| | | lupus erythematosus: results from | |
| | | EMBLEM, a phase IIb, randomised, | |
| | | double-blind, placebo-controlled, | |
| | | multicentre study. | |
| | | 2011 Sharkey R M, Govin . . . | |
| | | Epratuzumab-SN-38: a new antibody- | |
| | | drug conjugate for the therapy of | |
| | | hematologic malignancies. | |
| | | 2011 Micallef I N, Maur . . . | |
| | | Epratuzumab with rituximab, | |
| | | cyclophosphamide, doxorubicin, | |
| | | vincristine, and prednisone | |
| | | chemotherapy (ER-CHOP) in patients | |
| | | with previously untreated diffuse | |
| | | large B-cell lymphoma. | |
| | | 2011 Traczewski P, Rud . . . Treatment | |
| | | of systemic lupus erythematosus with | |
| | | epratuzumab. | |
| | | 2010 Daridon C, Blassf . . . Epratuzumab | |
| | | targeting of CD22 affects adhesion | |
| | | molecule expression and migration of | |
| | | B-cells in systemic lupus | |
| | | erythematosus. | |
| | | 2010 Gupta P, Goldenbe . . . Multiple | |
| | | signaling pathways induced by | |
| | | hexavalent, monospecific, anti-CD20 | |
| | | and hexavalent, bispecific, anti- | |
| | | CD20/CD22 humanized antibodies | |
| | | correlate with enhanced toxicity to B- | |
| | | cell lymphomas and leukemias. | |
| | | 2010 Morschhauser F, K . . . High rates | |
| | | of durable responses with anti-CD22 | |
| | | fractionated radioimmunotherapy: | |
| | | results of a multicenter, phase I/II | |
| | | study in non-Hodgkin's lymphoma. | |
| | | 2008 Leonard J P, Schus . . . Durable | |
| | | complete responses from therapy | |
| | | with combined epratuzumab and | |
| | | rituximab: final results from an | |
| | | international multicenter, phase 2 | |
| | | study in recurrent, indolent, non- | |
| | | Hodgkin lymphoma. | |
| | | 2008 Raetz E A, Cairo M . . . | |
| | | Chemoimmunotherapy reinduction | |
| | | with epratuzumab in children with | |
| | | acute lymphoblastic leukemia in | |
| | | marrow relapse: a Children's | |
| | | Oncology Group Pilot Study. | |
| | | 2008 Bodet-Milin C, Kr . . . Evaluation of | |
| | | response to fractionated | |
| | | radioimmunotherapy with 90Y- | |
| | | epratuzumab in non-Hodgkin's | |
| | | lymphoma by 18F-fluorodeoxyglucose | |
| | | positron emission tomography. | |
| | | 2007 Jacobi A M, Golden . . . Differential | |
| | | effects of epratuzumab on peripheral | |
| | | blood B cells of patients with systemic | |
| | | lupus erythematosus versus normal | |
| | | controls. | |
| | | 2006 Micallef I N, Kahl . . . A pilot study | |
| | | of epratuzumab and rituximab in | |
| | | combination with cyclophosphamide, | |
| | | doxorubicin, vincristine, and | |
| | | prednisone chemotherapy in patients | |
| | | with previously untreated, diffuse | |
| | | large B-cell lymphoma. | |
| | | 2006 Goldenberg D M Epratuzumab in | |
| | | the therapy of oncological and | |
| | | immunological diseases. | |
| | | 2006 Steinfeld S D, You . . . | |
| | | Epratuzumab (humanised anti-CD22 | |
| | | antibody) in autoimmune diseases. | |
| | | 2006 Carnahan J, Stein . . . | |
| | | Epratuzumab, a CD22-targeting | |
| | | recombinant humanized antibody | |
| | | with a different mode of action from | |
| | | rituximab. | |
| | | 2006 Dörner T, Kaufman . . . Initial | |
| | | clinical trial of epratuzumab | |
| | | (humanized anti-CD22 antibody) for | |
| | | immunotherapy of systemic lupus | |
| | | erythematosus. | |
| | | 2006 Remmele R L, Calla . . . Active | |
| | | dimer of Epratuzumab provides | |
| | | insight into the complex nature of an | |
| | | antibody aggregate. | |
| | | 2005 LindÃ ©n O, Hindor . . . Dose- | |
| | | fractionated radioimmunotherapy in | |
| | | non-Hodgkin's lymphoma using | |
| | | DOTA-conjugated, 90Y-radiolabeled, | |
| | | humanized anti-CD22 monoclonal | |
| | | antibody, epratuzumab. | |
| | | 2004 Leonard J P, Colem . . . | |
| | | Epratuzumab, a humanized anti-CD22 | |
| | | antibody, in aggressive non-Hodgkin's | |
| | | lymphoma: phase I/II clinical trial | |
| | | results. | |
| | | 2004 Stein R, Qu Z, Ch . . . | |
| | | Characterization of a new humanized | |
| | | anti-CD20 monoclonal antibody, | |
| | | IMMU-106, and Its use in | |
| | | combination with the humanized anti- | |
| | | CD22 antibody, epratuzumab, for the | |
| | | therapy of non-Hodgkin's lymphoma. | |
| | | 2003 Postema E J, Raema . . . Final | |
| | | results of a phase I | |
| | | radioimmunotherapy trial using | |
| | | (186)Re-epratuzumab for the | |
| | | treatment of patients with non- | |
| | | Hodgkin's lymphoma. | |
| | | 2003 Coleman M, Golden . . . | |
| | | Epratuzumab: targeting B-cell | |
| | | malignancies through CD22. | |
| | | 2003 Carnahan J, Wang . . . | |
| | | Epratuzumab, a humanized | |
| | | monoclonal antibody targeting CD22: | |
| | | characterization of in vitro properties. | |
| | | 2003 Leonard J P, Colem . . . Phase I/II | |
| | | trial of epratuzumab (humanized anti- | |
| | | CD22 antibody) in indolent non- | |
| | | Hodgkin's lymphoma. | |
| | | 2002 Leonard J P, Link B K | |
| | | Immunotherapy of non-Hodgkin's | |
| | | lymphoma with hLL2 (epratuzumab, | |
| | | an anti-CD22 monoclonal antibody) | |
| | | and Hu1D10 (apolizumab). | |
| | | 2000 Tsai D E, Schuster . . . Progressive | |
| | | intermediate-grade non-Hodgkin's | |
| | | lymphoma after high-dose therapy | |
| | | and autologous peripheral stem-cell | |
| | | transplantation: changing the natural | |
| | | history with monoclonal antibody | |
| | | therapy. | |
| | | AACR 2012 The in vitro effects of | |
| | | immobilized epratuzumab, a non- | |
| | | blocking anti-CD22 humanized | |
| | | monoclonal antibody, on malignant B | |
| | | cells Pankaj Gupta, Ros . . . | |
| | | AACR 2013 CD22-targeting | |
| | | epratuzumab mediates trogocytosis | |
| | | of multiple cell-surface markers on | |
| | | normal, malignant, and lupus B cells | |
| | | Edmund A. Rossi, . . . | |
| | | ACR/AHRP 2013 Sustained | |
| | | Improvements In Health-Related | |
| | | Quality Of Life In Patients With | |
| | | Systemic Lupus Erythematosus | |
| | | following Epratuzumab Treament: | |
| | | Results from a Phase IIb Trial and Its | |
| | | Open-Label Extension Strand, Vibeke | |
| | | F A . . . | |
| | | ACR/AHRP 2014 In Vivo Effects of | |
| | | Epratuzumab, a Monoclonal Antibody | |
| | | Targeting Human CD22, on B Cell | |
| | | Function in Human CD22 Knock-in | |
| | | (Huki) Mice Carolin Brandl, L . . . | |
| | | ACR/AHRP 2014 Correlation of | |
| | | Laboratory and Clinical Parameters | |
| | | with British Isles Lupus Assessment | |
| | | Group Response in an Open-Label | |
| | | Extension Study of Epratuzumab in | |
| | | Systemic Lupus Erythematosus | |
| | | Richard A. Furie, . . . | |
| | | ACR/AHRP 2014 Epratuzumab | |
| | | Induces Broad Inhibition of B Cell | |
| | | Receptor Proximal Signaling but Has | |
| | | Opposing Effects on Distal Signaling in | |
| | | B Cell Subsets: A Profile of Effects on | |
| | | Functional Immune Signaling By | |
| | | Single Cell Network Profiling Alison | |
| | | Maloney, D . . . | |
| | | ASH 2010 Consolidation Anti-CD22 | |
| | | Fractionated Radioimmunotherapy | |
| | | with 90Y Epratuzumab Tetraxetan | |
| | | Following R-CHOP In Elderly DLBCL | |
| | | Patients Françoise kraeber . . . | |
| | | ASH 2010 Epratuzumab (Humanized | |
| | | Anti-CD22 MAb) Conjugated with SN- | |
| | | 38, a New Antibody-Drug Conjugate | |
| | | (ADC) for the Treatment of | |
| | | Hematologic Tumors: Preclinical | |
| | | Studies Alone and In Combination | |
| | | with Veltuzumab, a Humanized Anti- | |
| | | CD20 MAb David M Goldenber . . . | |
| | | EULAR 2013 EPRATUZUMAB, AN | |
| | | ANTIBODY TARGETING CD22 ON B | |
| | | CELLS, INDUCES PHOSPHOPROTEIN | |
| | | CHANGES FOLLOWING B-CELL | |
| | | RECEPTOR ACTIVATION IN VITRO S. | |
| | | Lumb,, N. Tor . . . | |
| | | EULAR 2013 EPRATUZUMAB: | |
| | | SUSTAINED SAFETY PROFILE AND | |
| | | EFFECT ON CORTICOSTEROID USE ON | |
| | | LONG-TERM TREATMENT IN | |
| | | PATIENTS WITH MODERATE-TO- | |
| | | SEVERE SYSTEMIC LUPUS | |
| | | ERYTHEMATOSUS: RESULTS FROM AN | |
| | | OPEN-LABEL LONG-TERM EXTENSION | |
| | | STUDY (SL0008) D. J. Wallace,, . . . | |
| | | EULAR 2013 IMMUNOLOGIC | |
| | | RESPONSE TO LONG-TERM | |
| | | EPRATUZUMAB TREATMENT IN | |
| | | SL0008, AN OPEN-LABEL LONG-TERM | |
| | | EXTENSION STUDY IN PATIENTS WITH | |
| | | MODERATE-TO-SEVERE SYSTEMIC | |
| | | LUPUS ERYTHEMATOSUS V. Strand,, | |
| | | P. L . . . | |
| Eureka patent anti- | 2019 WO2019191704 | | Eureka |
| CD22 | 2019 US20210017280 | | |
| | 2018 US20210101954 | | |
| hLL2-Cys-PBD | | ASH 2016 hLL2-Cys-PBD, a New Site- | ADC Therapeutics |
| | | Specifically Conjugated, | Spirogen |
| | | Pyrrolobenzodiazepine (PBD) Dimer- | |
| | | Based Antibody Drug Conjugate (ADC) | |
| | | Targeting CD22-Expressing B-Cell | |
| | | Malignancies Francesca Zammarc . . . | |
| iCarTAB anti-CD22 | | 2016 NCT02794961 Phase 1/Phase 2 | iCarTAB Xuzhou |
| CAR | | CD22 Targeting CAR-T Therapy | Med.Coll. |
| | | Against B Cell Hematological | |
| | | Malignancies | |
| Immunomedics | 2017 US20170275363 | | Immunomedics |
| hRFB4 | 2017 U.S. Pat. No. 9,944,703 | | |
| | 2016 US20160376366 | | |
| | 2016 U.S. Pat. No. 9,701,748 | | |
| | 2016 US20160304611 | | |
| | 2015 U.S. Pat. No. 9,518,115 | | |
| | 2015 US20150239974 | | |
| | 2015 WO2015130416 | | |
| | 2015 U.S. Pat. No. 9,139,649 | | |
| Innobation Bio | 2022 WO2022181992 | | Innobation Bio |
| patent anti-CD22 | 2021 US20220204614 | | |
| | 2021 WO2022145739 | | |
| | 2021 WO2021235697 | | |
| | 2021 WO2021235696 | | |
| | 2021 WO2021246637 | | |
| inotuzumab | 2021 US20210371547 | 2020 NCT04456959 InO - A | Pfizer UCB Wyeth |
| ozogamicin | 2016 US20160326247 | Retrospective Study of UK Patients | |
| | 2014 US20140235835 | With Leukaemia | |
| | 2014 U.S. Pat. No. 9,351,986 | 2019 NCT03959085 Phase 3 | |
| | 2012 WO2013088304 | Inotuzumab Ozogamicin and Post- | |
| | 2012 US20120213804 | Induction Chemotherapy in Treating | |
| | 2012 U.S. Pat. No. 8,835,611 | Patients With High-Risk B-ALL, Mixed | |
| | 2011 US20110165659 | Phenotype Acute Leukemia, and B-LLy | |
| | 2011 U.S. Pat. No. 8,895,714 | 2019 NCT03991884 Phase 1 | |
| | 2005 U.S. Pat. No. 8,871,201 | Inotuzumab Ozogamicin and | |
| | 2005 WO2005089809 | Chemotherapy in Treating Patients | |
| | 2005 WO2005089807 | With Recurrent or Refractory B-cell | |
| | 2003 U.S. Pat. No. 8,747,857 | Acute Lymphoblastic Leukemia | |
| | 2003 US20040082764 | 2019 NCT03962465 Phase 1 Phase I | |
| | 2003 U.S. Pat. No. 7,355,011 | Study of Inotuzumab With | |
| | 2003 U.S. Pat. No. 8,153,768 | Augmented BFM Re-Induction for | |
| | | Younger Adults With | |
| | | Relapsed/Refractory B-cell ALL | |
| | | 2019 NCT03856216 Phase 2 | |
| | | Inotuzumab Ozogamicin and | |
| | | Chemotherapy in Treating Patients | |
| | | With Leukemia or Lymphoma | |
| | | Undergoing Stem Cell Transplant | |
| | | 2019 NCT03913559 Phase 2 | |
| | | Inotuzumab Ozogamicin for Children | |
| | | With MRD Positive CD22+ | |
| | | Lymphoblastic Leukemia | |
| | | 2019 NCT03571321 Phase 1 | |
| | | Ruxolitinib and Chemotherapy in | |
| | | Adolescents and Young Adults With | |
| | | Ph-like Acute Lymphoblastic Leukemia | |
| | | 2019 NCT03851081 Phase 1/Phase 2 | |
| | | Inotuzumab Ozogamicin and | |
| | | Vincristine Sulfate Liposome in | |
| | | Treating Patients With Relapsed or | |
| | | Refractory CD22+ B-cell Acute | |
| | | Lymphoblastic Leukemia | |
| | | 2019 NCT03628053 Phase 3 | |
| | | Tisagenlecleucel vs Blinatumomab or | |
| | | Inotuzumab for Patients With | |
| | | Relapsed/Refractory B-cell Precursor | |
| | | Acute Lymphoblastic Leukemia | |
| | | 2019 NCT03677596 Phase 4 A Study | |
| | | Of Two Inotuzumab Ozogamicin | |
| | | Doses in Relapsed/Refractory Acute | |
| | | Lymphoblastic Leukemia Transplant | |
| | | Eligible Patients | |
| | | 2018 NCT03739814 Phase 2 | |
| | | Inotuzumab Ozogamicin and | |
| | | Blinatumomab in Treating Patients | |
| | | With Newly Diagnosed, Recurrent, or | |
| | | Refractory CD22-Positive B-Lineage | |
| | | Acute Lymphoblastic Leukemia | |
| | | 2018 NCT03488225 Phase 2 Hyper- | |
| | | CVAD in Combination With | |
| | | Inotuzumab Ozogamicin as Frontline | |
| | | Therapy for Adults With Acute | |
| | | Lymphocytic Leukemia | |
| | | 2018 NCT03460522 Phase 2 | |
| | | Inotuzumab Ozogamicin and | |
| | | Conventional Chemotherapy In | |
| | | Patients Aged 56 Years and Older | |
| | | With ALL | |
| | | 2018 NCT03441061 Phase 2 Study of | |
| | | Inotuzumab Ozogamicin in Patients | |
| | | With B-cell Lineage Acute | |
| | | Lymphocytic Leukemia With Positive | |
| | | Minimal Residual Disease | |
| | | 2017 NCT03249870 Phase 2 Study of | |
| | | Inotuzumab Ozogamicin Combined to | |
| | | Chemotherapy in Older Patients With | |
| | | Philadelphia Chromosome-negative | |
| | | CD22+ B-cell Precursor ALL | |
| | | 2017 NCT03150693 Phase 3 | |
| | | Inotuzumab Ozogamicin and Frontline | |
| | | Chemotherapy in Treating Young | |
| | | Adults With Newly Diagnosed B Acute | |
| | | Lymphoblastic Leukemia | |
| | | 2017 NCT02981628 Phase 2 | |
| | | Inotuzumab Ozogamicin in Treating | |
| | | Younger Patients With Relapsed or | |
| | | Refractory CD22 Positive B Acute | |
| | | Lymphoblastic Leukemia | |
| | | 2017 NCT03094611 Phase 2 Study of | |
| | | Low Dose Inotuzumab Ozogamicin in | |
| | | Patients With Relapsed and | |
| | | Refractory CD22 Positive Acute | |
| | | Lymphocytic Leukemia | |
| | | 2017 NCT03104491 Phase 1/Phase 2 | |
| | | Inotuzumab Ozogamicin Post- | |
| | | Transplant For Acute Lymphocytic | |
| | | Leukemia | |
| | | 2015 NCT02311998 Phase 1/Phase 2 | |
| | | Phase I/II Study of Bosutinib in | |
| | | Combination With Inotuzumab | |
| | | Ozogamicin in CD22-positive | |
| | | Philadelphia-Chromosome (PC) | |
| | | Positive Acute Lymphoblastic | |
| | | Leukemia (ALL) and Chronic Myeloid | |
| | | Leukemia (CML) | |
| | | 2014 NCT01925131 Phase 1 S1312, | |
| | | Inotuzumab Ozogamicin and | |
| | | Combination Chemotherapy in | |
| | | Treating Patients With Relapsed or | |
| | | Refractory Acute Leukemia | |
| | | 2013 NCT01679119 Phase 2 | |
| | | Treatment of Patients With Diffuse | |
| | | Large B Cell Lymphoma Who Are Not | |
| | | Suitable for Anthracycline Containing | |
| | | Chemotherapy | |
| | | 2012 NCT01562990 Phase 1/Phase 2 | |
| | | Phase Ib/II Study of the Efficacy and | |
| | | Safety of the R-CMC544/R-GEMOX | |
| | | Combination in Diffuse Lage B-cell | |
| | | Lymphoma at First or Second Relapse | |
| | | 2012 NCT01664910 Phase 1/Phase 2 | |
| | | CMC-544 and Allogeneic | |
| | | Transplantation for CD22 Positive- | |
| | | Lymphoid Malignancies | |
| | | 2012 NCT01564784 Phase 3 A Study | |
| | | Of Inotuzumab Ozogamicin Versus | |
| | | Investigator's Choice Of | |
| | | Chemotherapy In Patients With | |
| | | Relapsed Or Refractory Acute | |
| | | Lymphoblastic Leukemia | |
| | | 2011 NCT01535989 Phase 1 Study of | |
| | | Inotuzumab Ozogamicin + | |
| | | Temsirolimus in Patients With | |
| | | Relapsed or Refractory CD22+ B-cell | |
| | | NHLymphoma | |
| | | 2011 NCT01371630 Phase 1/Phase 2 | |
| | | Study of the Combination of | |
| | | Inotuzumab Ozogamycin (CMC-544) | |
| | | With Low-intensity Chemotherapy in | |
| | | Patients With Acute Lymphoblastic | |
| | | Leukemia (ALL) | |
| | | 2011 NCT01363297 Phase 1 Study | |
| | | Evaluating Inotuzumab Ozogamicin In | |
| | | Acute Lymphocytic Leukemia | |
| | | 2011 NCT01232556 Phase 3 A Study | |
| | | Of Inotuzumab Ozogamicin Plus | |
| | | Rituximab For Relapsed/Refractory | |
| | | Aggressive Non-Hodgkin Lymphoma | |
| | | Patients Who Are Not Candidates For | |
| | | Intensive High-Dose Chemotherapy | |
| | | 2010 NCT01134575 Phase 1 CMC-544 | |
| | | in Relapsed Refractory Acute | |
| | | Lymphoblastic Leukemia (ALL) | |
| | | 2010 NCT01055496 Phase 1 Study | |
| | | Evaluating Chemotherapy in | |
| | | Combination With Inotuzumab | |
| | | Ozogamicin In Subjects With Non- | |
| | | Hodgkin's Lymphoma | |
| | | 2009 NCT00868608 Phase 2 Study | |
| | | Evaluating Inotuzumab Ozogamicin | |
| | | (CMC-544) In Indolent Non-Hodgkins | |
| | | Lymphoma | |
| | | 2009 NCT00867087 Phase 2 Study | |
| | | Evaluating Inotuzumab Ozogamicin | |
| | | (CMC-544) Plus Rituximab In Diffuse | |
| | | Large B-Cell Non-Hodgkin's | |
| | | Lymphoma | |
| | | 2008 NCT00724971 Phase 1 Study | |
| | | Evaluating the Safety and Tolerability | |
| | | of Combination Therapy Inotuzumab | |
| | | Ozogamicin (CMC-544) and Rituximab | |
| | | 2007 NCT00562965 Phase 3 Study | |
| | | Comparing Inotuzumab Ozogamicin In | |
| | | Combination With Rituximab Versus | |
| | | Defined Investigator's Choice In | |
| | | Follicular Non-Hodgkin's Lymphoma | |
| | | (NHL) | |
| | | 2007 NCT00717925 Phase 1 Study | |
| | | Evaluating Safety and Tolerability of | |
| | | Inotuzumab Ozogamicin (CMC-544) in | |
| | | Japanese Patients With B-cell Non- | |
| | | Hodgkin's Lymphoma (NHL) | |
| | | 2006 NCT00299494 Phase 1/Phase 2 | |
| | | Study Evaluating CMC-544 | |
| | | Administered In Combination With | |
| | | Rituximab In Subjects With Non- | |
| | | Hodgkin's Lymphoma (NHL) | |
| | | 2003 NCT00073749 Phase 1 Study | |
| | | Evaluating CMC-544 In B-Cell Non- | |
| | | Hodgkin's Lymphoma | |
| | | NCT03127605 N/A Inotuzumab | |
| | | Ozogamicin - PF-05208773 | |
| | | 2023 Agrawal V, Pourha . . . Post- | |
| | | transplant sinusoidal obstruction | |
| | | syndrome in adult patients with B-cell | |
| | | Acute Lymphoblastic Leukemia | |
| | | treated with pre-transplant | |
| | | Inotuzumab. | |
| | | 2022 Ceolin V, Brivio . . . Outcome of | |
| | | chimeric antigen receptor T-cell | |
| | | therapy following treatment with | |
| | | inotuzumab ozogamicin in children | |
| | | with relapsed or refractory acute | |
| | | lymphoblastic leukemia. | |
| | | 2022 Iwai T, Nishida M . . . | |
| | | Ultrasonographic monitoring of | |
| | | sinusoidal obstruction syndrome in | |
| | | patients treated with inotuzumab | |
| | | ozogamicin. | |
| | | 2022 Tomb P E, Shikdar . . . ALL-180 A | |
| | | Case of Refractory Philadelphia-Like | |
| | | Acute Lymphoblastic Leukemia | |
| | | Treated With | |
| | | Ruxolitinib/Blinatumomab and | |
| | | Ruxolitinib/Inotuzumab Ozogamicin | |
| | | Prior to Allogeneic Marrow | |
| | | Transplant. | |
| | | 2022 Maristany S, DuVa . . . Primary | |
| | | ovarian insufficiency secondary to | |
| | | chemotherapy with inotuzumab | |
| | | ozogamicin and other agents. | |
| | | 2022 Nakayama H, Ogawa . . . A phase I | |
| | | study of inotuzumab ozogamicin as a | |
| | | single agent in pediatric patients in | |
| | | Japan with relapsed/refractory CD22- | |
| | | positive acute lymphoblastic leukemia | |
| | | (INO-Ped-ALL-1). | |
| | | 2022 Stelljes M, Advan . . . Time to First | |
| | | Subsequent Salvage Therapy in | |
| | | Patients With Relapsed/Refractory | |
| | | Acute Lymphoblastic Leukemia | |
| | | Treated With Inotuzumab Ozogamicin | |
| | | in the Phase III INO-VATE Trial. | |
| | | 2022 Pennesi E, Michel . . . Inotuzumab | |
| | | ozogamicin as single agent in | |
| | | pediatric patients with relapsed and | |
| | | refractory acute lymphoblastic | |
| | | leukemia: results from a phase II trial. | |
| | | 2022 Short N J, Macaron . . . Dismal | |
| | | outcomes of patients with | |
| | | relapsed/refractory Philadelphia | |
| | | chromosome-negative B-cell acute | |
| | | lymphoblastic leukemia after failure | |
| | | of both inotuzumab ozogamicin and | |
| | | blinatumomab. | |
| | | 2022 Ohana Z, Serraes . . . Cytogenetic | |
| | | guided therapy using blinatumomab | |
| | | and inotuzumab ozogamicin in a | |
| | | patient with relapse/refractory acute | |
| | | lymphoblastic leukemia. | |
| | | 2022 Wudhikarn K, King . . . Outcomes | |
| | | of Relapsed B-Cell Acute | |
| | | Lymphoblastic Leukemia After | |
| | | Sequential Treatment with | |
| | | Blinatumomab and Inotuzumab. | |
| | | 2022 Sharplin K M, Marks D The | |
| | | treatment landscape for Relapsed | |
| | | Refractory B Acute Lymphoblastic | |
| | | Leukaemia (ALL). | |
| | | 2021 Sato M, Yasunaga . . . Successful | |
| | | diagnosis of veno-occlusive disease | |
| | | caused by inotuzumab ozogamicin | |
| | | through minimal-invasive | |
| | | angiography: a case report. | |
| | | 2021 Molica M, Mazzone . . . Durable | |
| | | Molecular Remission in an Elderly | |
| | | Patient Affected by Relapsed Ph'+ | |
| | | Acute Lymphoblastic Leukemia with | |
| | | T315I and Concomitant p190 and | |
| | | p210 Expression Achieved by | |
| | | Inotuzumab and Ponatinib. | |
| | | 2021 Brivio E, Locatel . . . A phase 1 | |
| | | study of inotuzumab ozogamicin in | |
| | | pediatric relapsed/refractory acute | |
| | | lymphoblastic leukemia (ITCC-059 | |
| | | study). | |
| | | 2021 Jabbour E, Paul S . . . The clinical | |
| | | development of antibody-drug | |
| | | conjugates - lessons from leukaemia. | |
| | | 2020 Li X, Zhou M, Qi . . . Efficacy and | |
| | | Safety of Inotuzumab Ozogamicin | |
| | | (CMC-544) for the Treatment of | |
| | | Relapsed/Refractory Acute | |
| | | Lymphoblastic Leukemia and Non- | |
| | | Hodgkin Lymphoma: A Systematic | |
| | | Review and Meta-Analysis. | |
| | | 2020 Stock W, Martinel . . . Efficacy of | |
| | | inotuzumab ozogamicin in patients | |
| | | with Philadelphia chromosome- | |
| | | positive relapsed/refractory acute | |
| | | lymphoblastic leukemia. | |
| | | 2020 Contreras C F, Hig . . . Clinical | |
| | | utilization of blinatumomab and | |
| | | inotuzumab immunotherapy in | |
| | | children with relapsed or refractory B- | |
| | | acute lymphoblastic leukemia. | |
| | | 2020 Jasinski S, De Lo . . . | |
| | | Immunotherapy in Pediatric B-Cell | |
| | | Acute Lymphoblastic Leukemia: | |
| | | Advances and Ongoing Challenges. | |
| | | 2020 Chen J, Haughey M . . . | |
| | | Characterization of the Relationship | |
| | | of Inotuzumab Ozogamicin Exposure | |
| | | With Efficacy and Safety End Points in | |
| | | Adults With Relapsed or Refractory | |
| | | Acute Lymphoblastic Leukemia. | |
| | | 2020 Fuster J L, Molino . . . | |
| | | Blinatumomab and inotuzumab for B | |
| | | cell precursor acute lymphoblastic | |
| | | leukaemia in children: a retrospective | |
| | | study from the Leukemia Working | |
| | | Group of the Spanish Society of | |
| | | Pediatric Hematology and Oncology | |
| | | (SEHOP). | |
| | | 2020 Badar T, Szabo A, . . . Real-World | |
| | | Outcomes of Adult B-Cell Acute | |
| | | Lymphocytic Leukemia Patients | |
| | | Treated With Inotuzumab | |
| | | Ozogamicin. | |
| | | 2019 Danylesko I, Chow . . . Treatment | |
| | | with anti CD19 chimeric antigen | |
| | | receptor T cells after antibody-based | |
| | | immunotherapy in adults with acute | |
| | | lymphoblastic leukemia. | |
| | | 2019 Paul M R, Wong V, . . . Treatment | |
| | | of Recurrent Refractory Pediatric Pre- | |
| | | B Acute Lymphoblastic Leukemia | |
| | | Using Inotuzumab Ozogamicin | |
| | | Monotherapy Resulting in CD22 | |
| | | Antigen Expression Loss as a | |
| | | Mechanism of Therapy Resistance. | |
| | | 2019 Jabbour E J, Sasak . . . Inotuzumab | |
| | | ozogamicin in combination with low- | |
| | | intensity chemotherapy (mini-HCVD) | |
| | | with or without blinatumomab versus | |
| | | standard intensive chemotherapy | |
| | | (HCVAD) as frontline therapy for older | |
| | | patients with Philadelphia | |
| | | chromosome-negative acute | |
| | | lymphoblastic leukemia: A propensity | |
| | | score analysis. | |
| | | 2019 Kantarjian H M, De . . . | |
| | | Inotuzumab ozogamicin versus | |
| | | standard of care in relapsed or | |
| | | refractory acute lymphoblastic | |
| | | leukemia: Final report and long-term | |
| | | survival follow-up from the | |
| | | randomized, phase 3 INO-VATE study. | |
| | | 2019 Corbacioglu S, Ja . . . Risk Factors | |
| | | for Development of and Progression | |
| | | of Hepatic Veno-occlusive | |
| | | Disease/Sinusoidal Obstruction | |
| | | Syndrome. | |
| | | 2019 Liu D, Zhao J, So . . . Clinical trial | |
| | | update on bispecific antibodies, | |
| | | antibody-drug conjugates, and | |
| | | antibody-containing regimens for | |
| | | acute lymphoblastic leukemia. | |
| | | 2019 Wynne J, Wright D . . . | |
| | | Inotuzumab: from preclinical | |
| | | development to success in B-cell | |
| | | acute lymphoblastic leukemia. | |
| | | 2019 Jabbour E, Advani . . . Prognostic | |
| | | implications of cytogenetics in adults | |
| | | with acute lymphoblastic leukemia | |
| | | treated with inotuzumab ozogamicin. | |
| | | 2018 Jain T, Litzow M R No free rides: | |
| | | management of toxicities of novel | |
| | | immunotherapies in ALL, including | |
| | | financial. | |
| | | 2018 Ma H, Sawas A Combining | |
| | | Biology and Chemistry for a New Take | |
| | | on Chemotherapy: Antibody-Drug | |
| | | Conjugates in Hematologic | |
| | | Malignancies. | |
| | | 2018 McDonald G B, Fres . . . Liver | |
| | | complications following treatment of | |
| | | hematologic malignancy with anti- | |
| | | cd22-calicheamicin (Inotuzumab | |
| | | Ozogamicin). | |
| | | 2018 Al-Salama Z T Inotuzumab | |
| | | Ozogamicin: A Review in | |
| | | Relapsed/Refractory B-Cell Acute | |
| | | Lymphoblastic Leukaemia. | |
| | | 2018 Kantarjian H M, Su . . . Patient- | |
| | | reported outcomes from a phase 3 | |
| | | randomized controlled trial of | |
| | | inotuzumab ozogamicin versus | |
| | | standard therapy for | |
| | | relapsed/refractory acute | |
| | | lymphoblastic leukemia. | |
| | | 2018 Short N J, Kantarj . . . Novel | |
| | | Therapies for Older Adults With Acute | |
| | | Lymphoblastic Leukemia. | |
| | | 2018 Foster J B, Maude SL New | |
| | | developments in immunotherapy for | |
| | | pediatric leukemia. | |
| | | 2017 Horvat T Z, Seddon . . . The ABCs of | |
| | | Immunotherapy for Adult Patients | |
| | | With B-Cell Acute Lymphoblastic | |
| | | Leukemia. | |
| | | 2017 Jabbour E, Ravand . . . Salvage | |
| | | Chemoimmunotherapy With | |
| | | Inotuzumab Ozogamicin Combined | |
| | | With Mini-Hyper-CVD for Patients | |
| | | With Relapsed or Refractory | |
| | | Philadelphia Chromosome-Negative | |
| | | Acute Lymphoblastic Leukemia: A | |
| | | Phase 2 Clinical Trial. | |
| | | 2017 ADC Approval for ALL Likely to | |
| | | Spur More Research. | |
| | | 2017 Lamb Y N Inotuzumab | |
| | | Ozogamicin: First Global Approval. | |
| | | 2017 Paul S, Rausch CR . . . Treatment | |
| | | of adult acute lymphoblastic leukemia | |
| | | with inotuzumab ozogamicin. | |
| | | 2017 Dang N H, Ogura M, . . . | |
| | | Randomized, phase 3 trial of | |
| | | inotuzumab ozogamicin plus | |
| | | rituximab versus chemotherapy plus | |
| | | rituximab for relapsed/refractory | |
| | | aggressive B-cell non-Hodgkin | |
| | | lymphoma. | |
| | | 2017 Valecha G K, Ibrah . . . Emerging | |
| | | Role of Immunotherapy in Precursor | |
| | | B-cell Lymphoblastic Leukemia. | |
| | | 2017 Thota S, Advani A Inotuzumab | |
| | | ozogamicin in relapsed b-cell acute | |
| | | lymphoblastic leukemia. | |
| | | 2016 Huguet F, Tavitian S Emerging | |
| | | biological therapies to treat acute | |
| | | lymphoblastic leukemia. | |
| | | 2016 Guffroy M, Falaha . . . Liver | |
| | | Microvascular Injury and | |
| | | Thrombocytopenia of Antibody- | |
| | | Calicheamicin Conjugates in | |
| | | Cynomolgus Monkeys - Mechanism | |
| | | and Monitoring. | |
| | | 2016 ADCs Show Promise in | |
| | | Leukemias. | |
| | | 2016 Kantarjian H M, De . . . | |
| | | Inotuzumab Ozogamicin versus | |
| | | Standard Therapy for Acute | |
| | | Lymphoblastic Leukemia. | |
| | | 2016 Betts A M, Haddish . . . Preclinical | |
| | | to Clinical Translation of Antibody- | |
| | | Drug Conjugates Using PK/PD | |
| | | Modeling: a Retrospective Analysis of | |
| | | Inotuzumab Ozogamicin. | |
| | | 2016 Dahl J, Marx K, J . . . Inotuzumab | |
| | | ozogamicin in the treatment of acute | |
| | | lymphoblastic leukemia. | |
| | | 2016 Morley N J, Marks D I Inotuzumab | |
| | | ozogamicin in the management of | |
| | | acute lymphoblastic leukaemia. | |
| | | 2016 George B, Kantarj . . . Role of | |
| | | inotuzumab ozogamicin in the | |
| | | treatment of relapsed/refractory | |
| | | acute lymphoblastic leukemia. | |
| | | 2015 Yilmaz M, Richard . . . The clinical | |
| | | potential of inotuzumab ozogamicin | |
| | | in relapsed and refractory acute | |
| | | lymphocytic leukemia. | |
| | | 2015 Ohanian M, Kantar . . . | |
| | | Inotuzumab ozogamicin in B-cell | |
| | | acute lymphoblastic leukemias and | |
| | | non-Hodgkin's lymphomas. | |
| | | 2015 Wagner-Johnston N . . . A Phase 2 | |
| | | Study of Inotuzumab Ozogamicin and | |
| | | Rituximab, Followed by Autologous | |
| | | Stem Cell Transplantation in Patients | |
| | | with Relapsed/Refractory Diffuse | |
| | | Large B-Cell Lymphoma. | |
| | | 2014 Jabbour E, O'Brie . . . Prognostic | |
| | | factors for outcome in patients with | |
| | | refractory and relapsed acute | |
| | | lymphocytic leukemia treated with | |
| | | inotuzumab ozogamicin, a cd22 | |
| | | monoclonal antibody. | |
| | | 2014 Shor B, Gerber H P . . . Preclinical | |
| | | and clinical development of | |
| | | inotuzumab-ozogamicin in | |
| | | hematological malignancies. | |
| | | 2013 Kantarjian H, Tho . . . Results of | |
| | | inotuzumab ozogamicin, a CD22 | |
| | | monoclonal antibody, in refractory | |
| | | and relapsed acute lymphocytic | |
| | | leukemia. | |
| | | 2013 Kebriaei P, Wilhe . . . Feasibility of | |
| | | allografting in patients with advanced | |
| | | acute lymphoblastic leukemia after | |
| | | salvage therapy with inotuzumab | |
| | | ozogamicin. | |
| | | 2012 Thomas X Inotuzumab | |
| | | ozogamicin in the treatment of B-cell | |
| | | acute lymphoblastic leukemia. | |
| | | 2012 Kantarjian H, Tho . . . Inotuzumab | |
| | | ozogamicin, an anti-CD22- | |
| | | calecheamicin conjugate, for | |
| | | refractory and relapsed acute | |
| | | lymphocytic leukaemia: a phase 2 | |
| | | study. | |
| | | 2012 Ogura M, Hatake K . . . Phase I | |
| | | Study of Anti-CD22 Immunoconjugate | |
| | | Inotuzumab Ozogamicin Plus | |
| | | Rituximab in Relapsed/Refractory B- | |
| | | cell Non-Hodgkin Lymphoma. | |
| | | 2011 de Vries J F, Zwaa . . . The novel | |
| | | calicheamicin-conjugated CD22 | |
| | | antibody inotuzumab ozogamicin | |
| | | (CMC-544) effectively kills primary | |
| | | pediatric acute lymphoblastic | |
| | | leukemia cells. | |
| | | 2010 Wong B Y, Dang N H Inotuzumab | |
| | | ozogamicin as novel therapy in | |
| | | lymphomas. | |
| | | 2010 Dijoseph J F, Doug . . . Preclinical | |
| | | anti-tumor activity of antibody- | |
| | | targeted chemotherapy with CMC- | |
| | | 544 (inotuzumab ozogamicin), a | |
| | | CD22-specific immunoconjugate of | |
| | | calicheamicin, compared with non- | |
| | | targeted combination chemotherapy | |
| | | with CVP or CHOP. | |
| | | 2010 Ogura M, Tobinai . . . Phase I | |
| | | study of inotuzumab ozogamicin | |
| | | (CMC-544) in Japanese patients with | |
| | | follicular lymphoma pretreated with | |
| | | rituximab-based therapy. | |
| | | 2010 Advani A, Coiffie . . . Safety, | |
| | | pharmacokinetics, and preliminary | |
| | | clinical activity of inotuzumab | |
| | | ozogamicin, a novel | |
| | | immunoconjugate for the treatment | |
| | | of B-cell non-Hodgkin's lymphoma: | |
| | | results of a phase I study. | |
| | | 2009 Takeshita A, Yama . . . CMC-544 | |
| | | (inotuzumab ozogamicin), an anti- | |
| | | CD22 immuno-conjugate of | |
| | | calicheamicin, alters the levels of | |
| | | target molecules of malignant B-cells. | |
| | | 2009 Takeshita A, Shin . . . CMC-544 | |
| | | (inotuzumab ozogamicin) shows less | |
| | | effect on multidrug resistant cells: | |
| | | analyses in cell lines and cells from | |
| | | patients with B-cell chronic | |
| | | lymphocytic leukaemia and | |
| | | lymphoma. | |
| | | 2007 Dijoseph J F, Doug . . . Therapeutic | |
| | | potential of CD22-specific antibody- | |
| | | targeted chemotherapy using | |
| | | inotuzumab ozogamicin (CMC-544) | |
| | | for the treatment of acute | |
| | | lymphoblastic leukemia. | |
| | | 2006 DiJoseph J F, Doug . . . Antitumor | |
| | | efficacy of a combination of CMC-544 | |
| | | (inotuzumab ozogamicin), a CD22- | |
| | | targeted cytotoxic immunoconjugate | |
| | | of calicheamicin, and rituximab | |
| | | against non-Hodgkin's B-cell | |
| | | lymphoma. | |
| | | ASH 2010 Phase 1 Study of Anti-CD22 | |
| | | Immunoconjugate Inotuzumab | |
| | | Ozogamicin (CMC-544) Plus Rituximab | |
| | | In Japanese Patients with | |
| | | Relapsed/Refractory B-Cell Non- | |
| | | Hodgkin Lymphoma Kiyohiko Hatake, | |
| | | . . . | |
| | | ASH 2010 Anti-CD22 | |
| | | Immunoconjugate Inotuzumab | |
| | | Ozogamicin (CMC-544) + Rituximab In | |
| | | Relapsed DLBCL Patients Followed by | |
| | | Stem Cell Transplantation: | |
| | | Preliminary Safety and Efficacy Nina | |
| | | Wagner-Johns . . . | |
| | | ASH 2011 Inotuzumab Ozogamicin | |
| | | (IO) Is An Effective Salvage Therapy | |
| | | That Allows for Allogeneic | |
| | | Hematopoietic Stem Cell | |
| | | Transplantation (HSCT) in Remission | |
| | | in Patients with Advanced Acute | |
| | | Lymphoblastic Leukemia (ALL) Partow | |
| | | Kebriaei, . . . | |
| | | ASH 2013 Phase I Study Of | |
| | | Inotuzumab Ozogamicin (InO) | |
| | | Combined With R-GDP For | |
| | | Relapsed/Refractory CD22+ B-Cell | |
| | | Non-Hodgkin Lymphoma (B-NHL) | |
| | | Randeep Sangha, A . . . | |
| | | ASH 2019 Inotuzumab Ozogamicin | |
| | | Post-Transplant for Acute | |
| | | Lymphocytic Leukemia Leland | |
| | | Metheny II . . . | |
| Iran U. Med. Sci | 2018 WO2020053634 | 2018 Faraji F, Tajik N . . . Development | Iran U.Med.Sci. |
| anti-CD22 | | and characterization of a camelid | |
| | | single domain antibody directed to | |
| | | human CD22 biomarker. | |
| JCAR018 | 2020 US20200283522 | 2014 NCT02315612 Phase 1 Anti- | Juno NCI |
| | 2013 US20150299317 | CD22 Chimeric Receptor T Cells in | |
| | 2013 WO2014065961 | Pediatric and Young Adults With | |
| | 2013 U.S. Pat. No. 10,072,078 | Recurrent or Refractory CD22- | |
| | 2012 US20140274909 | expressing B Cell Malignancies | |
| | 2012 WO2013059593 | 2013 Long A H, Haso W M, . . . Lessons | |
| | 2012 U.S. Pat. No. 9,868,774 | learned from a highly-active CD22- | |
| | | specific chimeric antigen receptor. | |
| Jiangsu Simcere | 2022 WO2022152282 | | Jiangsu Simcere |
| patent anti-CD22 | 2021 WO2022117032 | | |
| Kunming Sinoway | 2021 WO2022068899 | | Kunming Med.U. |
| patent anti-CD22 | | | |
| Medarex patent | 2016 US20170058031 | | Medarex |
| anti-CD22 | 2013 US20130230530 | | |
| | 2013 U.S. Pat. No. 9,499,632 | | |
| | 2007 WO2008070569 | | |
| | 2007 US20100143368 | | |
| | 2007 U.S. Pat. No. 8,481,683 | | |
| Medimmune | 2019 US20200165353 | 2016 Abuhay M, Kato J, . . . The HB22.7- | Medimmune |
| patent anti-CD22 | 2013 WO2014100443 | vcMMAE antibody-drug conjugate has | U.California |
| | 2013 U.S. Pat. No. 8,664,363 | efficacy against non-Hodgkin | |
| | 2012 WO2012170785 | lymphoma mouse xenografts with | |
| | 2012 US20140248278 | minimal systemic toxicity. | |
| | 2011 WO2012054679 | 2011 Tuscano J M, Ma Y, . . . The | |
| | 2010 US20110182887 | Bs20x22 anti-CD20-CD22 bispecific | |
| | 2007 WO2007103470 | antibody has more lymphomacidal | |
| | 2007 U.S. Pat. No. 7,829,086 | activity than do the parent antibodies | |
| | 2007 WO2007103469 | alone. | |
| | 2007 US20130028888 | ASH 2012 The HB22.7 Anti-CD22 | |
| | 2007 U.S. Pat. No. 8,389,688 | Monoclonal Antibody Is an Effective | |
| | | Platform for CD22-Targeted Antibody | |
| | | Drug Conjugates for Treatment of | |
| | | Lymphoma and Acute Lymphoblastic | |
| | | Leukemia Soames F. Boyle, . . . | |
| moxetumomab | 2009 WO2009149281 | 2019 NCT03805932 Phase 1 Title: | Medimmune |
| | | Moxetumomab Pasudotox (Lumoxiti) | |
| pasudotox | 2009 US20090305411 | and Rituximab (Rituxan) for Relapsed | |
| | 2009 U.S. Pat. No. 9,592,304 | Hairy Cell Leukemia | |
| | 2008 US20080292646 | | |
| | 2008 US20080254044 | 2015 NCT02338050 Phase 2 | |
| | 2008 WO2008097817 | | |
| | 2006 US20080193976 | Moxetumomab Pasudotox (CAT-8015, | |
| | | HA22) in Children With B-lineage | |
| | 2006 WO2007031741 | Acute Lymphoblastic Leukemia and | |
| | 2004 US20070189962 | Minimal Residual Disease Prior to | |
| | 2004 WO2005052006 | Allogeneic Hematopoietic Stem Cell | |
| | | Transplantation | |
| | 2003 US20040132657 | 2014 NCT02227108 Phase 2 A Phase | |
| | 2003 US20050191702 | 2, Multicenter Study in Pediatric | |
| | 2002 U.S. Pat. No. 6,881,718 | Subjects With Relapsed or Refractory | |
| | 2001 US20030017979 | Pediatric Acute Lymphoblastic | |
| | 2001 U.S. Pat. No. 6,723,538 | Leukemia (pALL) or Lymphoblastic | |
| | 2000 WO2001031020 | Lymphoma | |
| | EP2204385 | | |
| | JP2004508036 | 2013 NCT01829711 Phase 2 | |
| | JP2007536905 | Moxetumomab Pasudotox for | |
| | | Advanced Hairy Cell Leukemia | |
| | | 2010 NCT01086644 Phase 1/Phase 2 | |
| | | A Phase 1-2 Study of CAT-8015 in | |
| | | Adult Relapsed or Refractory B-Cell | |
| | | Non-Hodgkin Lymphoma and Chronic | |
| | | Lymphocytic Leukemia | |
| | | 2009 NCT01030536 Phase 1/2 Safety | |
| | | Study of CAT-8015 to Treat Advanced | |
| | | B-cell Non-Hodgkin Lymphoma and | |
| | | Chronic Lymphocytic Leukemia (NHL | |
| | | or CLL) | |
| | | 2008 NCT00659425 Phase 1 CAT-8015 | |
| | | in Children, Adolescents and Young | |
| | | Adults With Acute Lymphoblastic | |
| | | Leukemia or Non-Hodgkin's | |
| | | Lymphoma | |
| | JP2009511000 | 2007 NCT00515892 Phase 1 Safety | |
| | | Study of CAT-8015 Immunoxin in | |
| | | Patients With NHL With Advance | |
| | | Disease | |
| | | 2007 NCT00587015 Phase 1 A Phase I, | |
| | | Multicenter, Dose Escalation Study of | |
| | | CAT-8015 in Patients With Non- | |
| | | Hodgkin's Lymphoma (NHL) | |
| | | 2007 NCT00462189 Phase 1 Safety | |
| | | Study of CAT-8015 Immunooxin in | |
| | | Patients With HCL With Advance | |
| | | Disease | |
| | | 2007 NCT00457860 Phase 1 Safety | |
| | | Study of CAT-8015 Immunotoxin in | |
| | | Patients With CLL, PLL or SLL With | |
| | | Advance Disease | |
| | | 2006 NCT00587457 Phase 1 A Phase | |
| | | I, Multicenter, Dose Escalation Study | |
| | | of CAT-8015 in Patients With Chronic | |
| | | Leukemia | |
| | | 2006 NCT00586924 Phase 1 A Phase I, | |
| | | Multicenter Dose Escalation Study in | |
| | | Patients With Leukemia | |
| | | 2005 NCT00126646 Phase 1 BL22 | |
| | | Immunotoxin in Treating Patients | |
| | | With Refractory Chronic Lymphocytic | |
| | | Leukemia, Prolymphocytic Leukemia, | |
| | | or Non-Hodgkin's Lymphoma | |
| | | 2004 NCT00077493 Phase 1 BL22 | |
| | | Immunotoxin In Treating Young | |
| | | Patients With Relapsed or Refractory | |
| | | Acute Lymphoblastic Leukemia or | |
| | | Non-Hodgkin's Lymphoma | |
| | | 2003 NCT00074048 Phase 2 BL22 | |
| | | Immunotoxin in Treating Patients | |
| | | Previously Treated With Cladribine for | |
| | | Hairy Cell Leukemia | |
| | | 2020 Chihara D, Kreitm . . . Treatment | |
| | | of hairy cell leukemia. | |
| | | 2020 Kuruvilla D, Chia . . . Population | |
| | | Pharmacokinetics, Efficacy, and Safety | |
| | | of Moxetumomab Pasudotox in | |
| | | Patients With Relapsed or Refractory | |
| | | Hairy Cell Leukemia. | |
| | | 2019 Vainshtein I, Sun . . . A novel | |
| | | approach to assess domain specificity | |
| | | of anti-drug antibodies to | |
| | | moxetumomab pasudotox, an | |
| | | immunotoxin with two functional | |
| | | domains. | |
| | | 2019 Kreitman R J, Past . . . | |
| | | Contextualizing the Use of | |
| | | Moxetumomab Pasudotox in the | |
| | | Treatment of Relapsed or Refractory | |
| | | Hairy Cell Leukemia. | |
| | | 2019 Lin A Y, Dinner S N | |
| | | Moxetumomab pasudotox for hairy | |
| | | cell leukemia: preclinical | |
| | | development to FDA approval. | |
| | | 2019 Abou Dalle I, Rav . . . | |
| | | Moxetumomab pasudotox for the | |
| | | treatment of relapsed and/or | |
| | | refractory hairy cell leukemia. | |
| | | 2019 Janus A, Robak T Moxetumomab | |
| | | pasudotox for the treatment of hairy | |
| | | cell leukemia. | |
| | | 2019 King A C, Kabel C C . . . No Loose | |
| | | Ends: A Review of the | |
| | | Pharmacotherapy of Hairy Cell and | |
| | | Hairy Cell Leukemia Variant. | |
| | | 2018 Dhillon S Moxetumomab | |
| | | Pasudotox: First Global Approval. | |
| | | 2018 Wei J, Bera T K, L . . . Recombinant | |
| | | immunotoxins with albumin-binding | |
| | | domains have long half-lives and high | |
| | | antitumor activity. | |
| | | 2018 Kreitman R J, Tall . . . Minimal | |
| | | residual hairy cell leukemia | |
| | | eradication with moxetumomab | |
| | | pasudotox: phase I results and long- | |
| | | term follow-up. | |
| | | 2016 Pabst T M, Wendele . . . Camelid | |
| | | VHH affinity ligands enable separation | |
| | | of closely related biopharmaceuticals. | |
| | | 2016 Schneider A K, Vai . . . An | |
| | | immunoinhibition approach to | |
| | | overcome the impact of pre-existing | |
| | | antibodies on cut point establishment | |
| | | for immunogenicity assessment of | |
| | | moxetumomab pasudotox. | |
| | | 2015 Kreitman R J, Past . . . | |
| | | Immunoconjugates in the | |
| | | management of hairy cell leukemia. | |
| | | 2015 Robak T, Wolska A . . . Potential | |
| | | breakthroughs with investigational | |
| | | drugs for hairy cell leukemia. | |
| | | 2012 Kreitman R J, Tall . . . Phase I Trial | |
| | | of Anti-CD22 Recombinant | |
| | | Immunotoxin Moxetumomab | |
| | | Pasudotox (CAT-8015 or HA22) in | |
| | | Patients With Hairy Cell Leukemia. | |
| | | 2011 Kreitman R J, Aron . . . | |
| | | Recombinant immunotoxins and | |
| | | other therapies for | |
| | | relapsed/refractory hairy cell | |
| | | leukemia. | |
| | | 2011 Onda M, Beers R, . . . | |
| | | Recombinant immunotoxin against B- | |
| | | cell malignancies with no | |
| | | immunogenicity in mice by removal of | |
| | | B-cell epitopes. | |
| | | 2010 Bannister D, Popo . . . Epitope | |
| | | mapping and key amino acid | |
| | | identification of anti-CD22 | |
| | | immunotoxin CAT-8015 using hybrid | |
| | | {beta}-lactamase display. | |
| | | 2010 Mussai F, Campana . . . | |
| | | Cytotoxicity of the anti-CD22 | |
| | | immunotoxin HA22 (CAT-8015) | |
| | | against paediatric acute | |
| | | lymphoblastic leukaemia. | |
| | | 2010 Loomis K, Smith B . . . Specific | |
| | | targeting to B cells by lipid-based | |
| | | nanoparticles conjugated with a novel | |
| | | CD22-ScFv. | |
| | | 2009 Alderson R F, Krei . . . CAT-8015: a | |
| | | second-generation pseudomonas | |
| | | exotoxin A-based immunotherapy | |
| | | targeting CD22-expressing | |
| | | hematologic malignancies. | |
| | | 2008 Onda M, Beers R, . . . An | |
| | | immunotoxin with greatly reduced | |
| | | immunogenicity by identification and | |
| | | removal of B cell epitopes. | |
| | | 2006 Li Z, Mahesh SP, . . . Eradication | |
| | | of tumor colonization and invasion by | |
| | | a B cell-specific immunotoxin in a | |
| | | murine model for human primary | |
| | | intraocular lymphoma. | |
| | | 2005 Bang S, Nagata S, . . . HA22 | |
| | | (R490A) is a recombinant | |
| | | immunotoxin with increased | |
| | | antitumor activity without an increase | |
| | | in animal toxicity. | |
| | | ASCO 2012 Durability of complete | |
| | | remission by moxetumomab | |
| | | pasudotox (HA22 or CAT-8015) | |
| | | assessed by clone-specific real-time | |
| | | quantitative PCR (RQ-PCR). Robert J. | |
| | | Kreitma . . . | |
| | | ASH 2010 A Phase 1 Study of | |
| | | Moxetumomab Pasudotox, An Anti- | |
| | | CD22 Recombinant Immunotoxin, In | |
| | | Relapsed/Refractory Hairy Cell | |
| | | Leukemia (HCL): Updated Results | |
| | | Robert J. Kreitma . . . | |
| Mythic patent anti- | 2020 WO2021007361 | | Mythic Thera |
| CD22 | NICHOLS, Alexande . . . | | |
| | ANTIGEN-BINDING | | |
| | PROTEIN CONSTRUCTS | | |
| | AND USES THEREOF | | |
| | 2020 US20220288219 | | |
| | Nichols, Alexande . . . | | |
| | ANTIGEN-BINDING | | |
| | PROTEIN CONSTRUCTS | | |
| | AND USES THEREOF | | |
| | 2020 US20220324969 | | |
| | Nichols, Alexande . . . | | |
| | ANTIGEN-BINDING | | |
| | PROTEIN CONSTRUCTS | | |
| | AND USES THEREOF | | |
| Nanjing laso Bio | 2021 WO2021197483 | | laso |
| patent anti-CD22 | H U, Guang, YANG, . . . | | |
| CAR | FULLY HUMAN ANTI- | | |
| | HUMAN CD22 CHIMERIC | | |
| | ANTIGEN RECEPTOR | | |
| | AND APPLICATION | | |
| | THEREOF | | |
| Nanjing Legend Bio | 2021 WO2022012682 | | Nanjing Legend Bio |
| patent anti-CD22 | FAN, Xiaohu, ZHOU . . . | | |
| | CD22 BINDING | | |
| | MOLECULES AND USES | | |
| | THEREOF | | |
| | 2021 WO2022012681 | | |
| | FAN, Xiaohu, ZHOU . . . | | |
| | MULTISPECIFIC | | |
| | CHIMERIC ANTIGEN | | |
| | RECEPTORS AND USES | | |
| | THEREOF | | |
| NCI m971 | 2018 U.S. Pat. No. 10,703,816 | 2021 Ereño-Orbea J, Li . . . Structural | NCI |
| | Orentas, Rimas J . . .. | details of monoclonal antibody m971 | |
| | M971 chimeric antigen | recognition of the membrane- | |
| | receptors | proximal domain of CD22. | |
| | 2017 US20170145097 | 2009 Xiao X, Ho M, Zhu . . . | |
| | Dimitrov, Dimiter . . . | Identification and characterization of | |
| | HUMAN MONOCLONAL | fully human anti-CD22 monoclonal | |
| | ANTIBODIES SPECIFIC | antibodies. | |
| | FOR CD22 | 2004 Arndt M A, Krauss . . . Antigen | |
| | 2017 U.S. Pat. No. 10,494,435 | binding and stability properties of | |
| | Dimitrov, Dimiter . . . | non-covalently linked anti-CD22 | |
| | Human monoclonal | single-chain Fv dimers. | |
| | antibodies specific for | 2003 Arndt M A, Krauss . . . Generation | |
| | CD22 | of a highly stable, internalizing anti- | |
| | 2016 US20160145338 | CD22 single-chain Fv fragment for | |
| | Dimitrov, Dimiter . . . | targeting non-Hodgkin's lymphoma. | |
| | HUMAN MONOCLONAL | 2003 Krauss J, Arndt M . . . Specificity | |
| | ANTIBODIES SPECIFIC | grafting of human antibody | |
| | FOR CD22 | frameworks selected from a phage | |
| | 2016 U.S. Pat. No. 9,598,492 | display library: generation of a highly | |
| | Dimitrov, Dimiter. | stable humanized anti-CD22 single- | |
| | Human monoclonal | chain Fv fragment. | |
| | antibodies specific for | | |
| | CD22 | | |
| | 2013 US20130315921 | | |
| | Dimitrov, Dimiter . . . | | NCI |
| | HUMAN MONOCLONAL | | |
| | ANTIBODIES SPECIFIC | | |
| | FOR CD22 | | |
| | 2013 U.S. Pat. No. 9,279,019 | | |
| | Dimitrov, Dimiter . . . | | |
| | Human monoclonal | | |
| | antibodies specific for | | |
| | CD22 | | |
| | 2009 US20110020344 | | |
| | Dimitrov, Dimiter . . . | | |
| | HUMAN MONOCLONAL | | |
| | ANTIBODIES SPECIFIC | | |
| | FOR CD22 | | |
| | 2009 WO2009124109 | | |
| | DIMITROV, Dimiter . . . | | |
| | HUMAN MONOCLONAL | | |
| | ANTIBODIES SPECIFIC | | |
| | FOR CD22 | | |
| | 2009 U.S. Pat. No. 8,591,889 | | |
| | Dimitrov, Dimiter . . . | | |
| | Human monoclonal | | |
| | antibodies specific for | | |
| | CD22 | | |
| | 2003 U.S. Pat. No. 7,456,260 | | |
| | Rybak, Susanna M. | | |
| | Humanized antibody | | |
| | 2003 WO2003104425 | | |
| | RYBAK, Susanna; A . . . | | |
| | NOVEL STABLE ANTI- | | |
| | CD22 ANTIBODIES | | |
| | 2002 WO2003027135 | | |
| | PASTAN, Ira, H.; . . . | | |
| | MUTATED ANTI-CD22 | | |
| | ANTIBODIES WITH | | |
| | INCREASED AFFINITY TO | | |
| | CD22-EXPRESSING | | |
| | LEUKEMIA CELLS | | |
| NCI m972 | 2017 US20170145097 | 2009 Xiao X, Ho M, Zhu . . . | |
| | Dimitrov, Dimiter . . . | Identification and characterization of | |
| | HUMAN MONOCLONAL | fully human anti-CD22 monoclonal | |
| | ANTIBODIES SPECIFIC | antibodies. | |
| | FOR CD22 | | |
| | 2017 U.S. Pat. No. 10,494,435 | | |
| | Dimitrov, Dimiter . . . | | |
| | Human monoclonal | | |
| | antibodies specific for | | |
| | CD22 | | |
| | 2016 US20160145338 | | |
| | Dimitrov, Dimiter . . . | | |
| | HUMAN MONOCLONAL | | |
| | ANTIBODIES SPECIFIC | | |
| | FOR CD22 | | |
| | 2016 U.S. Pat. No. 9,598,492 | | |
| | Dimitrov, Dimiter . . . | | |
| | Human monoclonal | | |
| | antibodies specific for | | |
| | CD22 | | |
| | 2013 US20130315921 | | |
| | Dimitrov, Dimiter . . . | | |
| | HUMAN MONOCLONAL | | |
| | ANTIBODIES SPECIFIC | | |
| | FOR CD22 | | |
| | 2013 U.S. Pat. No. 9,279,019 | | |
| | Dimitrov, Dimiter . . . | | |
| | Human monoclonal | | |
| | antibodies specific for | | |
| | CD22 | | |
| | 2009 US20110020344 | | |
| | Dimitrov, Dimiter . . . | | |
| | HUMAN MONOCLONAL | | |
| | ANTIBODIES SPECIFIC | | |
| | FOR CD22 | | |
| | 2009 WO2009124109 | | |
| | DIMITROV, Dimiter . . . | | |
| | HUMAN MONOCLONAL | | |
| | ANTIBODIES SPECIFIC | | |
| | FOR CD22 | | |
| | 2009 U.S. Pat. No. 8,591,889 | | |
| | Dimitrov, Dimiter . . . | | |
| | Human monoclonal | | |
| | antibodies specific for | | |
| | CD22 | | |
| NIH patent anti- | 2019 WO2020014482 | | NIH |
| CD22 | DIMITROV, Dimiter . . . | | |
| | AFFINITY MATURED | | |
| | CD22-SPECIFIC | | |
| | MONOCLONAL | | |
| | ANTIBODY AND USES | | |
| | THEREOF | | |
| | 2019 US20210324074 | | |
| | Dimitrov, Dimiter . . . | | |
| | AFFINITY MATURED | | |
| | CD22-SPECIFIC | | |
| | MONOCLONAL | | |
| | ANTIBODY AND USES | | |
| | THEREOF | | |
| Novartis patent | 2021 US20220195010 | | Novartis |
| anti-CD22 CAR | Bitter, Hans; Bor . . . CD20 | | |
| | THERAPIES, CD22 | | |
| | THERAPIES, AND | | |
| | COMBINATION | | |
| | THERAPIES WITH A | | |
| | CD19 CHIMERIC | | |
| | ANTIGEN RECEPTOR | | |
| | (CAR)- EXPRESSING CELL | | |
| | 2020 WO2021108613 | | |
| | ENGELS, Boris, GU . . . | | |
| | CD19 AND CD22 | | |
| | CHIMERIC ANTIGEN | | |
| | RECEPTORS AND USES | | |
| | THEREOF | | |
| | 2019 WO2020069409 | | |
| | ISAACS, Randi, FR . . . | | |
| | CD19 CHIMERIC | | |
| | ANTIGEN RECEPTOR | | |
| | (CAR) AND CD22 CAR | | |
| | COMBINATION | | |
| | THERAPIES | | |
| | 2019 WO2020069405 | | |
| | GRUPP, Stephan CD22 | | |
| | CHIMERIC ANTIGEN | | |
| | RECEPTOR (CAR) | | |
| | THERAPIES | | |
| | 2019 US20190292238 | | |
| | Bitter, Hans; Bor . . . CD20 | | |
| | THERAPIES, CD22 | | |
| | THERAPIES, AND | | |
| | COMBINATION | | |
| | THERAPIES WITH A | | |
| | CD19 CHIMERIC | | |
| | ANTIGEN RECEPTOR | | |
| | (CAR)- EXPRESSING CELL | | |
| | 2017 WO2018067992 | | |
| | BRANNETTI, Barbar . . . | | |
| | CHIMERIC ANTIGEN | | |
| | RECEPTORS FOR THE | | |
| | TREATMENT OF CANCER | | |
| NRC Canada patent | 2021 WO2022020945 | | NRC Canada |
| anti-CD22 | MCCOMB, Scott, WE . . . | | |
| | ANTI-CD22 SINGLE | | |
| | DOMAIN ANTIBODIES | | |
| | AND THERAPEUTIC | | |
| | CONSTRUCTS | | |
| Pasteur Inst. Iran | 2022 Mohammadi Z, | | Pasteur Inst. Iran |
| anti-CD22 | Enay . . . A Novel Anti- | | |
| | CD22 scFv.Bim Fusion | | |
| | Protein Effectively | | |
| | Induces Apoptosis in | | |
| | Malignant B cells and | | |
| | Promotes Cytotoxicity. | | |
| | 2017 Agha Amiri S, Zar . . . | | |
| | Expression Optimization | | |
| | of Anti-CD22 scFv- | | |
| | Apoptin Fusion Protein | | |
| | Using Experimental | | |
| | Design Methodology | | |
| | 2014 Zarei N, Vaziri B . . . | | |
| | High efficient expression | | |
| | of a functional | | |
| | humanized single-chain | | |
| | variable fragment (scFv) | | |
| | antibody against CD22 | | |
| | in Pichia pastoris. | | |
| PersonGen anti- | | 2022 Zhang T, Wang T, . . . Nanobody- | PersonGen |
| CD22 CAR | | based anti-CD22-chimeric antigen | |
| | | receptor T cell immunotherapy | |
| | | exhibits improved remission against | |
| | | B-cell acute lymphoblastic leukemia. | |
| pinatuzumab | 2017 US20170232113 | 2020 Yu S F, Lee D W, Zh . . . An anti- | Genentech |
| vedotin | 2017 US20170290920 | CD22-seco-CBI-Dimer ADC for the | Medimmune Seattle |
| | 2017 US20170326248 | treatment of non-Hodgkin lymphoma | Genetics |
| | 2017 US20170306040 | that provides a longer duration of | |
| | 2016 US20160279260 | response than auristatin ADCs in | |
| | 2016 US20160279261 | preclinical models. | |
| | 2016 WO2016205176 | 2019 Morschhauser F, F . . . | |
| | 2016 U.S. Pat. No. 10,124,069 | Polatuzumab vedotin or pinatuzumab | |
| | 2014 WO2014177617 | vedotin plus rituximab in patients | |
| | 2014 US20150010540 | with relapsed or refractory non- | |
| | 2013 WO2014011520 | Hodgkin lymphoma: final results from | |
| | 2013 WO2014011518 | a phase 2 randomised study | |
| | 2013 US20140030279 | (ROMULUS). | |
| | 2013 US20140030281 | 2017 Ereño-Orbea J, Si . . . Structural | |
| | 2013 US20140127197 | Basis of Enhanced Crystallizability | |
| | 2013 U.S. Pat. No. 8,968,741 | Induced by a Molecular Chaperone | |
| | 2012 U.S. Pat. No. 8,394,607 | for Antibody Antigen-Binding | |
| | 2010 US20110142859 | Fragments. | |
| | 2007 WO2007140371 | 2012 NCT01691898 Phase 2 A Study | |
| | 2007 US20080050310 | of DCDT2980S in Combination With | |
| | 2007 U.S. Pat. No. 8,524,865 | MabThera/Rituxan or DCDS4501A in | |
| | | Combination With MabThera/Rituxan | |
| | | in Patients With Non-Hodgkin's | |
| | | Lymphoma | |
| | | 2010 NCT01209130 Phase 1 A Study | |
| | | of the Safety and Pharmacokinetics of | |
| | | Escalating Doses of DCDT2980S in | |
| | | Patients With Relapsed or Refractory | |
| | | B-Cell Non-Hodgkin's Lymphoma and | |
| | | Chronic Lymphocytic Leukemia | |
| | | 2016 Fuh F K, Looney C, . . . Anti-CD22 | |
| | | and anti-CD79b antibody-drug | |
| | | conjugates preferentially target | |
| | | proliferating B cells. | |
| | | 2016 Ma Y, Khojasteh S . . . Antibody | |
| | | drug conjugates differentiate uptake | |
| | | and DNA alkylation of | |
| | | pyrrolobenzodiazepines in tumors | |
| | | and organs of xenograft mice. | |
| | | 2016 Advani R H, Lebovi . . . Phase I | |
| | | study of the anti-CD22 antibody-drug | |
| | | conjugate pinatuzumab vedotin | |
| | | with/without rituximab in patients | |
| | | with relapsed/refractory B-cell non- | |
| | | Hodgkin's lymphoma. | |
| | | 2015 Yu S F, Zheng B, G . . . A novel anti- | |
| | | CD22 anthracycline-based antibody- | |
| | | drug conjugate (ADC) that overcomes | |
| | | resistance to auristatin based ADCs. | |
| | | 2015 Pfeifer M, Zheng . . . Anti-CD22 | |
| | | and anti-CD79B antibody drug | |
| | | conjugates are active in different | |
| | | molecular diffuse large B-cell | |
| | | lymphoma subtypes. | |
| | | 2013 Li D, Achilles-Po . . . DCDT2980S, | |
| | | an anti-CD22-Monomethyl Auristatin | |
| | | E antibody-drug conjugate, is a | |
| | | potential treatment for non-Hodgkins | |
| | | Lymphoma. | |
| | | ASCO 2014 Preliminary results of a | |
| | | phase II randomized study | |
| | | (ROMULUS) of polatuzumab vedotin | |
| | | (PoV) or pinatuzumab vedotin (PIV) | |
| | | plus rituximab (RTX) in patients (Pts) | |
| | | with relapsed/refractory (R/R) non- | |
| | | Hodgkin lymphoma (NHL) Franck | |
| | | Morschhaus . . . | |
| | | ASH 2010 Targeted Depletion of B- | |
| | | Cell Subsets by Anti-CD22 and Anti- | |
| | | CD79bAntibody Drug Conjugates: | |
| | | Illumination of the Mechanism of | |
| | | Action through Pharmacodynamic | |
| | | Biomarkers Franklin Fuh*, Ca . . . | |
| | | ASH 2012 A Phase I Study of | |
| | | DCDT2980S, an Antibody-Drug | |
| | | Conjugate (ADC) Targeting CD22, in | |
| | | Relapsed or Refractory B-Cell Non- | |
| | | Hodgkin's Lymphoma (NHL) Ranjana | |
| | | Advani, M . . . | |
| | | ASH 2013 Final Results Of a Phase I | |
| | | Study Of The Anti-CD22 Antibody- | |
| | | Drug Conjugate (ADC) DCDT2980S | |
| | | With Or Without Rituximab (RTX) In | |
| | | Patients (Pts) With Relapsed Or | |
| | | Refractory (R/R) B-Cell Non-Hodgkin's | |
| | | Lymphoma (NHL) Ranjana Advani, | |
| | | M . . . | |
| RFB4 | 2012 US20120258106 | 2015 Weber T, Böttiche . . . High | Abiogen NIH |
| | 2012 U.S. Pat. No. 8,809,502 | treatment efficacy by dual targeting | |
| | 2004 U.S. Pat. No. 7,982,011 | of Burkitt's lymphoma xenografted | |
| | 2002 U.S. Pat. No. 7,355,012 | mice with a (177)Lu-based CD22- | |
| | | specific radioimmunoconjugate and | |
| | | rituximab. | |
| | | 2010 Wayne A S, Kreitma . . . Anti-CD22 | |
| | | immunotoxin RFB4(dsFv)-PE38 (BL22) | |
| | | for CD22-positive hematologic | |
| | | malignancies of childhood: preclinical | |
| | | studies and phase I clinical trial. | |
| | | 2005 Vallera D A, Brech . . . | |
| | | Radioimmunotherapy of CD22- | |
| | | expressing Daudi tumors in nude mice | |
| | | with a 90Y-labeled anti-CD22 | |
| | | monoclonal antibody. | |
| Shenzhen Feipeng | 2021 WO2022042494 | | Shenzhen Feipeng |
| Bio patent anti- | D U, Xiaolong, PEN . . . | | Bio |
| CD22 | CD22 ANTIBODY AND | | |
| | APPLICATION THEREOF | | |
| Sloan-Kettering | 2020 WO2020185763 | | Sloan-Kettering |
| patent anti-CD22 | CHEUNG, Nai-Kong . . . | | |
| | CD22 ANTIBODIES AND | | |
| | METHODS OF USING | | |
| | THE SAME | | |
| | 2020 US20220177581 | | |
| | CHEUNG, Nai-Kong . . . | | |
| | CD22 ANTIBODIES AND | | |
| | METHODS OF USING | | |
| | THE SAME | | |
| SM03 | 2022 WO2023284710 | 2017 NCT04312815 Phase 3 A Study | SinoMab |
| | LEUNG, Shui-On | Assessing the Efficacy and Safety of | |
| | METHODS OF TREATING | SM03 in Patients With Active | |
| | NEUROLOGICAL | Rheumatoid Arthritis Receiving MTX | |
| | DISEASES | 2014 NCT04192617 Phase 2 A Study | |
| | 2019 WO2020078454 | to Evaluate the Safety and Efficacy of | |
| | LEUNG, Shui-On | SM03 in Patients With Rheumatoid | |
| | METHOD OF | Arthritis Receiving Methotrexate | |
| | MODULATING | 2012 NCT04704492 Phase 1 A Study | |
| | AUTOIMMUNITY BY | of the Pharmacokinetics and Safety of | |
| | DISRUPTING CIS-LIGAND | SM03 in Patients With Rheumatoid | |
| | BINDING OF SIGLEC | Arthritis | |
| | TYPE ANTIGENS | 2022 Wong K L, Li Z, Ma . . . SM03, an | |
| | 2019 WO2020078453 | Anti-CD22 Antibody, Converts Cis-to- | |
| | LEUNG, Shui-On | Trans Ligand Binding of CD22 against | |
| | METHODS OF TREATING | α2,6-Linked Sialic Acid Glycans and | |
| | RHEUMATOID | Immunomodulates Systemic | |
| | ARTHRITIS | Autoimmune Diseases. | |
| | 2019 US20210363246 | 2021 Li J, Li M, Wu D, . . . SM03, an anti- | |
| | Leung, Shui-On; Method | human CD22 monoclonal antibody, | |
| | of Modulating | for active rheumatoid arthritis: a | |
| | Autoimmunity by | phase II randomized, double-blind, | |
| | Disrupting cis-Ligand | placebo-controlled study. | |
| | Binding of Siglec Type | 2016 Zhao Q, Chen X, L . . . | |
| | Antigens | Pharmacokinetics, | |
| | 2019 US20210380685 | Pharmacodynamics and Preliminary | |
| | Leung, Shui-On; | Observations for Clinical Activity and | |
| | Methods of Treating | Safety of Multiple Doses of Human | |
| | Rheumatoid Arthritis | Mouse Chimeric Anti-CD22 | |
| | 2016 U.S. Pat. No. 10,613,099 | Monoclonal Antibody (SM03) in | |
| | Leung, Shui-on (F . . . Cell | Chinese Patients with Systemic Lupus | |
| | lines expressing surface | Erythematosus. | |
| | bound anti-idiotype | | |
| | antibodies against anti- | | |
| | CD22 antibodies and | | |
| | uses thereof | | |
| | 2016 US20160363597 | | |
| | LEUNG, Shui-on; CELL | | |
| | LINES EXPRESSING | | |
| | SURFACE BOUND ANTI- | | |
| | IDIOTYPE ANTIBODIES | | |
| | AGAINST ANTI-CD22 | | |
| | ANTIBODIES AND USES | | |
| | THEREOF | | |
| TAC-001 | | 2022 NCT05399654 Phase 1/Phase 2 | Tallac |
| | | A Dose Escalation and Expansion | |
| | | Study of TAC-001 in Patients With | |
| | | Select Advanced or Metastatic Solid | |
| | | Tumors | |
| TeneoBio patent | 2018 WO2019126756 | | TeneoBio |
| anti-CD22 | ALDRED, Shelley F . . . | | |
| | HEAVY CHAIN | | |
| | ANTIBODIES BINDING | | |
| | TO CD22 | | |
| | 2018 US20210095022 | | |
| | Force Aldred, She . . . | | |
| | HEAVY CHAIN | | |
| | ANTIBODIES BINDING | | |
| | TO CD22 | | |
| TRPH-222 | 2021 US20220220198 | 2018 NCT03682796 Phase 1 Study of | Catalent Redwood |
| | 2019 US20190352395 | TRPH-222 in Patients With Relapsed | Triphase |
| | 2018 WO2019118411 | and/or Refractory B-Cell Lymphoma | |
| | 2018 US20190201541 | 2017 Drake P M, Carlson . . . CAT-02- | |
| | 2016 WO2017083306 | 106, a site-specifically conjugated | |
| | 2016 US20200246480 | anti-CD22 antibody bearing an MDR1- | |
| | 2015 US20160229911 | resistant maytansine payload yields | |
| | 2015 U.S. Pat. No. 10,259,871 | excellent efficacy and safety in | |
| | 2015 US20150344573 | preclinical models. | |
| | 2013 WO2014014821 | ASH 2017 Trph-222, a Novel Anti- | |
| | 2013 US20140161870 | CD22 Antibody Drug Conjugate (ADC), | |
| | 2013 U.S. Pat. No. 9,181,343 | Has Signficant Anti-Tumor Activity in | |
| | | NHL Xenografts and Is Well Tolerated | |
| | | in Non-Human Primates Ann | |
| | | Maclaren, PhD . . . | |
| U. Penn. anti-CD22 | | 2015 NCT02588456 N/A Pilot Study of | U.Penn. |
| CAR | | Autologous Anti-CD22 Chimeric | |
| | | Antigen Receptor Redirected T Cells In | |
| | | Patients With Chemotherapy | |
| | | Resistant Or Refractory Acute | |
| | | Lymphoblastic Leukemia | |
| UCB patent anti- | 2021 WO2021224629 | | Autolus UCB |
| CD22 CAR | 2019 WO2019220109 | | |
| | 2015 WO2016102965 | | |
| Xi'an Yufan Bio | 2022 WO2022262783 | | Xi'an Yufan Bio |
| patent anti-CD22 | | | |
| Cellectis patent | 2021 WO2022023529 | | Cellectis |
| anti-CD20/CD22 | | | |
| CAR | | | |
| Elpis Bio patent | 2022 WO2022159653 | | Elpis Bio |
| anti-CD19/CD22 | | | |
| Guangdong Zhaotai | 2020 WO2022011846 | | Guangdong Zhaotai |
| Invivo Bio patent | | | Invivo Bio |
| anti-CD19/CD22 | | | |
| CAR | | | |
| Janssen patent | 2022 US20220306738 | | Janssen Biotech |
| anti-CD22/CD79B | 2022 WO2022201052 | | |
| JNJ-75348780 | | 2020 NCT04540796 Phase 1 A Study | Janssen Biotech |
| | | of JNJ-75348780 in Participants With | |
| | | Non-Hodgkin Lymphoma (NHL) and | |
| | | Chronic Lymphocytic Leukemia (CLL) | |
| Lentigen patent | 2022 US20220324967 | | Lentigen NIH |
| anti-CD22/CD19 | Schneider, Dina; . . . | | |
| CAR | Compositions and | | |
| | Methods for Treating | | |
| | Cancer with Anti- | | |
| | CD19/CD22 | | |
| | Immunotherapy | | |
| | 2021 WO2022099026 | | |
| | 2021 US20210379110 | | |
| | 2020 US20210171633 | | |
| | 2020 U.S. Pat. No. 11,242,389 | | |
| | 2020 US20210171632 | | |
| | 2020 US20200155661 | | |
| | 2019 US20200087396 | | |
| | 2019 WO2020069184 | | |
| | 2019 U.S. Pat. No. 10,822,412 | | |
| | 2018 US20190099476 | | |
| | 2018 WO2019079249 | | |
| | 2018 U.S. Pat. No. 10,543,263 | | |
| Nanjing laso Bio | 2021 WO2022002154 | | laso |
| patent anti-CD22/ | YANG, Yongkun, H U . . . | | |
| CD19 CAR | FULLY HUMANIZED | | |
| | BISPECIFIC CHIMERIC | | |
| | ANTIGEN RECEPTOR | | |
| | TARGETING CD19 AND | | |
| | CD22 AND USE THEREOF | | |
| NIH patent anti- | 2019 WO2020014482 | | NIH |
| CD19/CD22 CAR | 2016 WO2016149578 | | |
| REGN5837 | 2019 WO2020132066 | 2023 NCT05685173 Phase 1 Study to | Regeneron |
| | 2019 US20200239576 | Assess the Safety and Tolerability of | |
| | 2019 U.S. Pat. No. 11,396,544 | REGN5837 in Combination With | |
| | | Odronextamab in Patients With | |
| | | Relapsed or Refractory Aggressive B- | |
| | | cell Non-Hodgkin Lymphoma | |
| | | 2022 Wei J, Montalvo-O . . . CD22- | |
| | | targeted CD28 bispecific antibody | |
| | | enhances antitumor efficacy of | |
| | | odronextamab in refractory diffuse | |
| | | large B cell lymphoma models. | |
| TeneoBio patent | 2020 WO2020252366 | | TeneoBio |
| anti-CD3/CD22 | 2020 US20210047402 | | |
| UCB patent anti- | 2016 WO2017009474 | | UCB |
| CD22/CD79b | 2016 WO2017009476 | | |
| | 2015 WO2016009030 | | |
| Amgen patent anti- | 2022 WO2022234102 | | Amgen |
| CD20/CD22/CD3 | PANZER, Marc CD20 | | |
| | AND CD22 TARGETING | | |
| | ANTIGEN-BINDING | | |
| | MOLECULES FOR USE IN | | |
| | PROLIFERATIVE | | |
| | DISEASES | | |
| Peking U. anti- | | 2022 Zhao L, Li S, Wei . . . A novel | Peking U. |
| CD19/CD22/CD3 | | CD19/CD22/CD3 trispecific antibody | |
| | | enhances therapeutic efficacy and | |
| | | overcomes immune escape against B- | |
| | | ALL. | |

d. BCMA CAR

In some embodiments, the additional CAR is a BCMA CAR (“BCMA-CAR”), and in these embodiments, the polycistronic vector comprises an expression cassette that contains a nucleotide sequence encoding a BCMA CAR. BCMA is a tumor necrosis family receptor (TNFR) member expressed on cells of the B cell lineage, with the highest expression on terminally differentiated B cells or mature B lymphocytes. BCMA is involved in mediating the survival of plasma cells for maintaining long-term humoral immunity. The expression of BCMA has been recently linked to a number of cancers, such as multiple myeloma, Hodgkin's and non-Hodgkin's lymphoma, various leukemias, and glioblastoma. In some embodiments, the method comprises administering to a subject a BCMA-targeting CAR therapy in combination with a gamma secretase inhibitor (GSI). Any suitable GSI known in the art, in view of the present disclosure, can be used. Examples of suitable GSIs include, but are not limited to, those disclosed in US2020/0055948, which is incorporated by reference in its entirety.

In some embodiments, the BCMA CAR may comprise a signal peptide, an extracellular binding domain that specifically binds BCMA, a hinge domain, a transmembrane domain, an intracellular costimulatory domain, and/or an intracellular signaling domain in tandem.

In some embodiments, the signal peptide of the BCMA CAR comprises a CD8a signal peptide. In some embodiments, the CD8a signal peptide comprises or consists of an amino acid sequence set forth in SEQ ID NO:6 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:6. In some embodiments, the signal peptide comprises an IgK signal peptide. In some embodiments, the IgK signal peptide comprises or consists of an amino acid sequence set forth in SEQ ID NO:7 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:7. In some embodiments, the signal peptide comprises a GMCSFR-α or CSF2RA signal peptide. In some embodiments, the GMCSFR-α or CSF2RA signal peptide comprises or consists of an amino acid sequence set forth in SEQ ID NO:8 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:8.

In some embodiments, the extracellular binding domain of the BCMA CAR is specific to BCMA, for example, human BCMA. The extracellular binding domain of the BCMA CAR can be codon-optimized for expression in a host cell or to have variant sequences to increase functions of the extracellular binding domain.

In some embodiments, the extracellular binding domain comprises an immunogenically active portion of an immunoglobulin molecule, for example, an scFv. In some embodiments, the extracellular binding domain of the BCMA CAR is derived from an antibody specific to BCMA, including, for example, belantamab, erlanatamab, teclistamab, LCAR-B38M, and ciltacabtagene. In any of these embodiments, the extracellular binding domain of the BCMA CAR can comprise or consist of the V_{H}, the V_{L}, and/or one or more CDRs of any of the antibodies.

In some embodiments, the extracellular binding domain of the BCMA CAR comprises an scFv derived from C11D5.3, a murine monoclonal antibody as described in Carpenter et al., Clin. Cancer Res. 19(8):2048-2060 (2013). See also PCT Application Publication No. WO2010/104949. The C11D5.3-derived scFv may comprise the heavy chain variable region (V_{H}) and the light chain variable region (V_{L}) of C11D5.3 connected by the Whitlow linker, the amino acid sequences of which is provided in Table 17 below. In some embodiments, the BCMA-specific extracellular binding domain comprises or consists of an amino acid sequence set forth in SEQ ID NO:63, 64, or 68, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:63, 64, or 68. In some embodiments, the BCMA-specific extracellular binding domain may comprise one or more CDRs having amino acid sequences set forth in SEQ ID NOs: 65-67 and 69-71. In some embodiments, the BCMA-specific extracellular binding domain may comprise a light chain with one or more CDRs having amino acid sequences set forth in SEQ ID NOs: 65-67. In some embodiments, the BCMA-specific extracellular binding domain may comprise a heavy chain with one or more CDRs having amino acid sequences set forth in SEQ ID NOs: 69-71. In any of these embodiments, the BCMA-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the BCMA CAR comprises or consists of the one or more CDRs as described herein.

In some embodiments, the extracellular binding domain of the BCMA CAR comprises an scFv derived from another murine monoclonal antibody, C12A3.2, as described in Carpenter et al., Clin. Cancer Res. 19(8):2048-2060 (2013) and PCT Application Publication No. WO2010/104949, the amino acid sequence of which is also provided in Table 17 below. In some embodiments, the BCMA-specific extracellular binding domain comprises or consists of an amino acid sequence set forth in SEQ ID NO:72, 73, or 77, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:72, 73, or 77. In some embodiments, the BCMA-specific extracellular binding domain may comprise one or more CDRs having amino acid sequences set forth in SEQ ID NOs: 74-76 and 78-80. In some embodiments, the BCMA-specific extracellular binding domain may comprise a light chain with one or more CDRs having amino acid sequences set forth in SEQ ID NOs: 74-76. In some embodiments, the BCMA-specific extracellular binding domain may comprise a heavy chain with one or more CDRs having amino acid sequences set forth in SEQ ID NOs: 78-80. In any of these embodiments, the BCMA-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the BCMA CAR comprises or consists of the one or more CDRs as described herein.

In some embodiments, the extracellular binding domain of the BCMA CAR comprises a murine monoclonal antibody with high specificity to human BCMA, referred to as BB2121 in Friedman et al., Hum. Gene Ther. 29(5):585-601 (2018)). See also, PCT Application Publication No. WO2012163805.

In some embodiments, the extracellular binding domain of the BCMA CAR comprises single variable fragments of two heavy chains (VHH) that can bind to two epitopes of BCMA as described in Zhao et al., J. Hematol. Oncol. 11(1):141 (2018), also referred to as LCAR-B38M. See also, PCT Application Publication No. WO2018/028647.

In some embodiments, the extracellular binding domain of the BCMA CAR comprises a fully human heavy-chain variable domain (FHVH) as described in Lam et al., Nat. Commun. 11(1):283 (2020), also referred to as FHVH33. See also, PCT Application Publication No. WO2019/006072. The amino acid sequences of FHVH33 and its CDRs are provided in Table 173 below. In some embodiments, the BCMA-specific extracellular binding domain comprises or consists of an amino acid sequence set forth in SEQ ID NO:81 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:81. In some embodiments, the BCMA-specific extracellular binding domain may comprise one or more CDRs having amino acid sequences set forth in SEQ ID NOs: 82-84. In any of these embodiments, the BCMA-specific extracellular binding domain may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the BCMA CAR comprises or consists of the one or more CDRs as described herein.

In some embodiments, the extracellular binding domain of the BCMA CAR comprises an scFv derived from CT103A (or CAR0085) as described in U.S. Pat. No. 11,026,975 B2, the amino acid sequence of which is provided in Table 17 below. In some embodiments, the BCMA-specific extracellular binding domain comprises or consists of an amino acid sequence set forth in SEQ ID NO:118, 119, or 123, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO: 118, 119, or 123. In some embodiments, the BCMA-specific extracellular binding domain may comprise one or more CDRs having amino acid sequences set forth in SEQ ID NOs: 120-122 and 124-126. In some embodiments, the BCMA-specific extracellular binding domain may comprise a light chain with one or more CDRs having amino acid sequences set forth in SEQ ID NOs: 120-122. In some embodiments, the BCMA-specific extracellular binding domain may comprise a heavy chain with one or more CDRs having amino acid sequences set forth in SEQ ID NOs: 124-126. In any of these embodiments, the BCMA-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the BCMA CAR comprises or consists of the one or more CDRs as described herein.

Additionally, CARs and binders directed to BCMA have been described in U.S. Application Publication Nos. 2020/0246381 A1 and 2020/0339699 A1, the entire contents of each of which are incorporated by reference herein.

TABLE 17
Exemplary sequences of anti-BCMA binder and components

| SEQ ID NO: | Amino Acid Sequence | Description |
|---|---|---|
| 63 | DIVLTQSPASLAMSLGKRATISCRASESVSVIGAH | Anti-BCMA C11D5.3 scFv |
| | LIHWYQQKPGQPPKLLIYLASNLETGVPARFSGS | entire sequence, with |
| | GSGTDFTLTIDPVEEDDVAIYSCLQSRIFPRTFGG | Whitlow linker |
| | GTKLEIKGSTSGSGKPGSGEGSTKGQIQLVQSGP | |
| | ELKKPGETVKISCKASGYTFTDYSINWVKRAPGK | |
| | GLKWMGWINTETREPAYAYDFRGRFAFSLETSA | |
| | STAYLQINNLKYEDTATYFCALDYSYAMDYWGQ | |
| | GTSVTVSS | |
| 64 | DIVLTQSPASLAMSLGKRATISCRASESVSVIGAH | Anti-BCMA C11D5.3 scFv |
| | LIHWYQQKPGQPPKLLIYLASNLETGVPARFSGS | light chain variable region |
| | GSGTDFTLTIDPVEEDDVAIYSCLQSRIFPRTFGG | |
| | GTKLEIK | |
| 65 | RASESVSVIGAHLIH | Anti-BCMA C11D5.3 scFv |
| | | light chain CDR1 |
| 66 | LASNLET | Anti-BCMA C11D5.3 scFv |
| | | light chain CDR2 |
| 67 | LQSRIFPRT | Anti-BCMA C11D5.3 scFv |
| | | light chain CDR3 |
| 68 | QIQLVQSGPELKKPGETVKISCKASGYTFTDYSIN | Anti-BCMA C11D5.3 scFv |
| | WVKRAPGKGLKWMGWINTETREPAYAYDFRG | heavy chain variable |
| | RFAFSLETSASTAYLQINNLKYEDTATYFCALDYSY | region |
| | AMDYWGQGTSVTVSS | |
| 69 | DYSIN | Anti-BCMA C11D5.3 scFv |
| | | heavy chain CDR1 |
| 70 | WINTETREPAYAYDFRG | Anti-BCMA C11D5.3 scFv |
| | | heavy chain CDR2 |
| 71 | DYSYAMDY | Anti-BCMA C11D5.3 scFv |
| | | heavy chain CDR3 |
| 72 | DIVLTQSPPSLAMSLGKRATISCRASESVTILGSHL | Anti-BCMA C12A3.2 scFv |
| | IYWYQQKPGQPPTLLIQLASNVQTGVPARFSGS | entire sequence, with |
| | GSRTDFTLTIDPVEEDDVAVYYCLQSRTIPRTFGG | Whitlow linker |
| | GTKLEIKGSTSGSGKPGSGEGSTKGQIQLVQSGP | |
| | ELKKPGETVKISCKASGYTFRHYSMNWVKQAPG | |
| | KGLKWMGRINTESGVPIYADDFKGRFAFSVETS | |
| | ASTAYLVINNLKDEDTASYFCSNDYLYSLDFWGQ | |
| | GTALTVSS | |
| 73 | DIVLTQSPPSLAMSLGKRATISCRASESVTILGSHL | Anti-BCMA C12A3.2 scFv |
| | IYWYQQKPGQPPTLLIQLASNVQTGVPARFSGS | light chain variable region |
| | GSRTDFTLTIDPVEEDDVAVYYCLQSRTIPRTFGG | |
| | GTKLEIK | |
| 74 | RASESVTILGSHLIY | Anti-BCMA C12A3.2 scFv |
| | | light chain CDR1 |
| 75 | LASNVQT | Anti-BCMA C12A3.2 scFv |
| | | light chain CDR2 |
| 76 | LQSRTIPRT | Anti-BCMA C12A3.2 scFv |
| | | light chain CDR3 |
| 77 | QIQLVQSGPELKKPGETVKISCKASGYTFRHYSM | Anti-BCMA C12A3.2 scFv |
| | NWVKQAPGKGLKWMGRINTESGVPIYADDFK | heavy chain variable |
| | GRFAFSVETSASTAYLVINNLKDEDTASYFCSNDY | region |
| | LYSLDFWGQGTALTVSS | |
| 78 | HYSMN | Anti-BCMA C12A3.2 scFv |
| | | heavy chain CDR1 |
| 79 | RINTESGVPIYADDFKG | Anti-BCMA C12A3.2 scFv |
| | | heavy chain CDR2 |
| 80 | DYLYSLDF | Anti-BCMA C12A3.2 scFv |
| | | heavy chain CDR3 |
| 81 | EVQLLESGGGLVQPGGSLRLSCAASGFTFSSYAM | Anti-BCMA FHVH33 entire |
| | SWVRQAPGKGLEWVSSISGSGDYIYYADSVKGR | sequence |
| | FTISRDISKNTLYLQMNSLRAEDTAVYYCAKEGT | |
| | GANSSLADYRGQGTLVTVSS | |
| 82 | GFTFSSYA | Anti-BCMA FHVH33 CDR1 |
| 83 | ISGSGDYI | Anti-BCMA FHVH33 CDR2 |
| 84 | AKEGTGANSSLADY | Anti-BCMA FHVH33 CDR3 |
| 118 | DIQMTQSPSSLSASVGDRVTITCRASQSISSYLN | Anti-BCMA CT103A scFv |
| | WYQQKPGKAPKLLIYAASSLQSGVPSRFSGSGSG | entire sequence, with |
| | TDFTLTISSLQPEDFATYYCQQKYDLLTFGGGTKV | Whitlow linker |
| | EIKGSTSGSGKPGSGEGSTKGQLQLQESGPGLVK | |
| | PSETLSLTCTVSGGSISSSSYYWGWIRQPPGKGLE | |
| | WIGSISYSGSTYYNPSLKSRVTISVDTSKNQFSLKL | |
| | SSVTAADTAVYYCARDRGDTILDVWGQGTMVT | |
| | VSS | |
| 119 | DIQMTQSPSSLSASVGDRVTITCRASQSISSYLN | Anti-BCMA CT103A scFv |
| | WYQQKPGKAPKLLIYAASSLQSGVPSRFSGSGSG | light chain variable region |
| | TDFTLTISSLQPEDFATYYCQQKYDLLTFGGGTKV | |
| | EIK | |
| 120 | QSISSY | Anti-BCMA CT103A scFv |
| | | light chain CDR1 |
| 121 | AAS | Anti-BCMA CT103A scFv |
| | | light chain CDR2 |
| 122 | QQKYDLLT | Anti-BCMA CT103A scFv |
| | | light chain CDR3 |
| 123 | QLQLQESGPGLVKPSETLSLTCTVSGGSISSSSYY | Anti-BCMA CT103A scFv |
| | WGWIRQPPGKGLEWIGSISYSGSTYYNPSLKSRV | heavy chain variable |
| | TISVDTSKNQFSLKLSSVTAADTAVYYCARDRGD | region |
| | TILDVWGQGTMVTVSS | |
| 124 | GGSISSSSYY | Anti-BCMA CT103A scFv |
| | | heavy chain CDR1 |
| 125 | ISYSGST | Anti-BCMA CT103A scFv |
| | | heavy chain CDR2 |
| 126 | ARDRGDTILDV | Anti-BCMA CT103A scFv |
| | | heavy chain CDR3 |

In some embodiments, the hinge domain of the BCMA CAR comprises a CD8a hinge domain, for example, a human CD8a hinge domain. In some embodiments, the CD8a hinge domain comprises or consists of an amino acid sequence set forth in SEQ ID NO:9 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:9. In some embodiments, the hinge domain comprises a CD28 hinge domain, for example, a human CD28 hinge domain. In some embodiments, the CD28 hinge domain comprises or consists of an amino acid sequence set forth in SEQ ID NO:10 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:10. In some embodiments, the hinge domain comprises an IgG4 hinge domain, for example, a human IgG4 hinge domain. In some embodiments, the IgG4 hinge domain comprises or consists of an amino acid sequence set forth in SEQ ID N0:11 or SEQ ID NO:12, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID N0:11 or SEQ ID NO:12. In some embodiments, the hinge domain comprises a IgG4 hinge-Ch2-Ch3 domain, for example, a human IgG4 hinge-Ch2-Ch3 domain. In some embodiments, the IgG4 hinge-Ch2-Ch3 domain comprises or consists of an amino acid sequence set forth in SEQ ID NO:13 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:13.

In some embodiments, the transmembrane domain of the BCMA CAR comprises a CD8a transmembrane domain, for example, a human CD8a transmembrane domain. In some embodiments, the CD8a transmembrane domain comprises or consists of an amino acid sequence set forth in SEQ ID NO:14 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:14. In some embodiments, the transmembrane domain comprises a CD28 transmembrane domain, for example, a human CD28 transmembrane domain. In some embodiments, the CD28 transmembrane domain comprises or consists of an amino acid sequence set forth in SEQ ID NO:15 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:15.

In some embodiments, the intracellular costimulatory domain of the BCMA CAR comprises a 4-1BB costimulatory domain, for example, a human 4-1BB costimulatory domain. In some embodiments, the 4-1BB costimulatory domain comprises or consists of an amino acid sequence set forth in SEQ ID NO:16 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:16. In some embodiments, the intracellular costimulatory domain comprises a CD28 costimulatory domain, for example, a human CD28 costimulatory domain. In some embodiments, the CD28 costimulatory domain comprises or consists of an amino acid sequence set forth in SEQ ID NO:17 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:17.

In some embodiments, the intracellular signaling domain of the BCMA CAR comprises a CD3 zeta (ζ) signaling domain, for example, a human CD3ζ signaling domain. In some embodiments, the CD3ζ signaling domain comprises or consists of an amino acid sequence set forth in SEQ ID NO:18 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:18.

In some embodiments, the polycistronic vector comprises an expression cassette that contains a nucleotide sequence encoding a BCMA CAR, including, for example, a BCMA CAR comprising any of the BCMA-specific extracellular binding domains as described, the CD8a hinge domain of SEQ ID NO:9, the CD8a transmembrane domain of SEQ ID NO:14, the 4-1BB costimulatory domain of SEQ ID NO:16, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof. In any of these embodiments, the BCMA CAR may additionally comprise a signal peptide (e.g., a CD8a signal peptide) as described.

In some embodiments, the polycistronic vector comprises an expression cassette that contains a nucleotide sequence encoding a BCMA CAR, including, for example, a BCMA CAR comprising any of the BCMA-specific extracellular binding domains as described, the CD8a hinge domain of SEQ ID NO:9, the CD8a transmembrane domain of SEQ ID NO:14, the CD28 costimulatory domain of SEQ ID NO:17, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof. In any of these embodiments, the BCMA CAR may additionally comprise a signal peptide as described.

In some embodiments, the polycistronic vector comprises an expression cassette that contains a nucleotide sequence encoding a BCMA CAR as set forth in SEQ ID NO:127 or is at least 80% identical (e.g., at least 80%, at least 85%, at least 90%, at least 95%, at least 96%, at least 97%, at least 98%, at least 99%, or 100% identical) to the nucleotide sequence set forth in SEQ ID NO:127 (see Table 18). The encoded BCMA CAR has a corresponding amino acid sequence set forth in SEQ ID NO:128 or is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:128, with the following components: CD8a signal peptide, CT103A scFv (V_{L}—Whitlow linker-V_{H}), CD8a hinge domain, CD8a transmembrane domain, 4-1BB costimulatory domain, and CD3ζ signaling domain.

In some embodiments, the polycistronic vector comprises an expression cassette that contains a nucleotide sequence encoding a commercially available embodiment of BCMA CAR, including, for example, idecabtagene vicleucel (ide-cel, also called bb2121). In some embodiments, the polycistronic vector comprises an expression cassette that contains a nucleotide sequence encoding idecabtagene vicleucel or portions thereof. Idecabtagene vicleucel comprises a BCMA CAR with the following components: the BB2121 binder, CD8a hinge domain, CD8a transmembrane domain, 4-11B1 costimulatory domain, and CD3 signaling domain.

TABLE 18
Exemplary sequences of BCMA CARs

| SEQ ID NO: | Sequence | Description |
|---|---|---|
| 127 | atggccttaccagtgaccgccttgctcctgccgctggccttgctgctcca | Exemplary BCMA |
| | cgccgccaggccggacatccagatgacccagtctccatcctccctgtct | CAR nucleotide |
| | gcatctgtaggagacagagtcaccatcacttgccgggcaagtcagagc | sequence |
| | attagcagctatttaaattggtatcagcagaaaccagggaaagcccct | |
| | aagctcctgatctatgctgcatccagtttgcaaagtggggtcccatcaa | |
| | ggttcagtggcagtggatctgggacagatttcactctcaccatcagcag | |
| | tctgcaacctgaagattttgcaacttactactgtcagcaaaaatacgac | |
| | ctcctcacttttggcggagggaccaaggttgagatcaaaggcagcacc | |
| | agcggctccggcaagcctggctctggcgagggcagcacaaagggaca | |
| | gctgcagctgcaggagtcgggcccaggactggtgaagccttcggaga | |
| | ccctgtccctcacctgcactgtctctggtggctccatcagcagtagtagt | |
| | tactactggggctggatccgccagcccccagggaaggggctggagtg | |
| | gattgggagtatctcctatagtgggagcacctactacaacccgtccctc | |
| | aagagtcgagtcaccatatccgtagacacgtccaagaaccagttctcc | |
| | ctgaagctgagttctgtgaccgccgcagacacggcggtgtactactgc | |
| | gccagagatcgtggagacaccatactagacgtatggggtcagggtac | |
| | aatggtcaccgtcagctcattcgtgcccgtgttcctgcccgccaaaccta | |
| | ccaccacccctgcccctagacctcccaccccagccccaacaatcgcca | |
| | gccagcctctgtctctgcggcccgaagcctgtagacctgctgccggcgg | |
| | agccgtgcacaccagaggcctggacttcgcctgcgacatctacatctg | |
| | ggcccctctggccggcacctgtggcgtgctgctgctgagcctggtgatc | |
| | accctgtactgcaaccaccggaacaaacggggcagaaagaaactcct | |
| | gtatatattcaaacaaccatttatgagaccagtacaaactactcaaga | |
| | ggaagatggctgtagctgccgatttccagaagaagaagaaggaggat | |
| | gtgaactgagagtgaagttcagcagatccgccgacgcccctgcctacc | |
| | agcagggacagaaccagctgtacaacgagctgaacctgggcagacg | |
| | ggaagagtacgacgtgctggacaagcggagaggccgggaccccgag | |
| | atgggcggaaagcccagacggaagaacccccaggaaggcctgtata | |
| | acgaactgcagaaagacaagatggccgaggcctacagcgagatcgg | |
| | catgaagggcgagcggaggcgcggcaagggccacgatggcctgtacc | |
| | agggcctgagcaccgccaccaaggacacctacgacgccctgcacatg | |
| | caggccctgccccccaga | |
| 128 | MALPVTALLLPLALLLHAARPDIQMTQSPSSLSASVGDR | Exemplary BCMA |
| | VTITCRASQSISSYLNWYQQKPGKAPKLLIYAASSLQSGV | CAR amino acid |
| | PSRFSGSGSGTDFTLTISSLQPEDFATYYCQQKYDLLTFG | sequence |
| | GGTKVEIKGSTSGSGKPGSGEGSTKGQLQLQESGPGLVK | |
| | PSETLSLTCTVSGGSISSSSYYWGWIRQPPGKGLEWIGSI | |
| | SYSGSTYYNPSLKSRVTISVDTSKNQFSLKLSSVTAADTAV | |
| | YYCARDRGDTILDVWGQGTMVTVSSFVPVFLPAKPTTT | |
| | PAPRPPTPAPTIASQPLSLRPEACRPAAGGAVHTRGLDF | |
| | ACDIYIWAPLAGTCGVLLLSLVITLYCNHRNKRGRKKLLYI | |
| | FKQPFMRPVQTTQEEDGCSCRFPEEEEGGCELRVKFSRS | |
| | ADAPAYQQGQNQLYNELNLGRREEYDVLDKRRGRDPE | |
| | MGGKPRRKNPQEGLYNELQKDKMAEAYSEIGMKGERR | |
| | RGKGHDGLYQGLSTATKDTYDALHMQALPPR | |

In some embodiments, a polynucleotide provided herein comprises a nucleotide sequence encoding a BCMA CAR, a variable domain of a BCMA CAR, or a set of CDRs (HCDR 1, 2, and 3 and LCDR 1, 2, and 3) that encodes a BCMA CAR as set forth in TABLE 19 below or a variable domain of a BCMA CAR or a set of CDRs (HCDR 1, 2, and 3 and LCDR 1, 2, and 3) therefrom. In some embodiments, a polynucleotide provided herein comprises a nucleotide sequence encoding a BCMA CAR, a variable domain of a BCMA CAR, or a set of CDRs (HCDR 1, 2, and 3 and LCDR 1, 2, and 3) that having at least 80% (e.g., at least 80%, at least 85%, at least 90%, at least 95%, at least 96%, at least 97%, at least 98%, at least 99%, or 100% identical) to a BCMA CAR as set forth in TABLE 19 below or a variable domain of a BCMA CAR or a set of CDRs (HCDR 1, 2, and 3 and LCDR 1, 2, and 3) therefrom, respectively.

TABLE 19
Exemplary BCMA antigen binding domains

| Antibody Name | Patents | Publications | Company |
|---|---|---|---|
| 2A9-MICA | | 2020 Wang Y, Li H, Xu . . . | China Pharm. U. |
| | | BCMA-targeting Bispecific | |
| | | Antibody That | |
| | | Simultaneously Stimulates | |
| | | NKG2D-enhanced Efficacy | |
| | | Against Multiple Myeloma. | |
| ABL Bio patent anti- | 2019 WO2020004934 | | ABL Bio |
| BCMA | PARK, Kyungjin, C . . . ANTI- | | |
| | BCMA ANTIBODY AND | | |
| | USE THEREOF | | |
| | 2019 US20210284749 | | |
| | PARK, Kyungjin; C . . . Anti- | | |
| | BCMA Antibody And Use | | |
| | Thereof | | |
| Ablink patent anti-BCMA | 2020 WO2021043169 | | Ablink |
| | LIU, Jianghai, ZE . . . | | |
| | ANTIBODY THAT BINDS | | |
| | SPECIFICALLY TO B-CELL | | |
| | MATURATION ANTIGEN | | |
| | AND USE THEREOF | | |
| ALLO-605 | 2019 WO2020112796 | 2021 NCT05000450 Phase | Allogene |
| | CHANG, David, BAL . . . | 1/Phase 2 Safety and | |
| | CHIMERIC ANTIGEN | Efficacy of ALLO-605 an | |
| | RECEPTORS TARGETING | Anti-BCMA Allogeneic CAR | |
| | B-CELL MATURATION | T Cell Therapy in Patients | |
| | ANTIGEN AND METHODS | With Relapsed/Refractory | |
| | OF USE THEREOF | Multiple Myeloma | |
| AMG 224 | 2019 WO2020069303 | 2015 NCT02561962 Phase | Amgen |
| | KIELCZEWSKA, Agni . . . | 1 A Phase 1 Study in | |
| | ANTIBODIES AGAINST | Subjects With Relapsed or | |
| | SOLUBLE BCMA | Refractory Multiple | |
| | 2019 U.S. Pat. No. 11,505,614 | Myeloma | |
| | Kielczewska, Agni . . . | 2020 Lee HC, Raje NS, . . . | |
| | Antibodies binding to | Letter phase 1 study of the | |
| | soluble BCMA | anti-BCMA antibody-drug | |
| | 2015 US20170165373 | conjugate AMG 224 in | |
| | ARMITAGE, Richard . . . | patients with | |
| | BCMA ANTIGEN BINDING | relapsed/refractory | |
| | PROTEINS | multiple myeloma. | |
| | 2015 US20190008974 | | |
| | ARMITAGE, Richard . . . | | |
| | BCMA ANTIGEN BINDING | | |
| | PROTEINS | | |
| | 2015 U.S. Pat. No. 10,220,090 | | |
| | Armitage, Richard . . . | | |
| | BCMA Antigen binding | | |
| | proteins | | |
| | 2013 WO2014089335 | | |
| | ARMITAGE, Richard . . . | | |
| | BCMA ANTIGEN BINDING | | |
| | PROTEINS | | |
| | 2013 US20140161828 | | |
| | ARMITAGE, Richard . . . | | |
| | BCMA ANTIGEN BINDING | | |
| | PROTEINS | | |
| | 2013 US20150344583 | | |
| | ARMITAGE, Richard . . . | | |
| | BCMA ANTIGEN BINDING | | |
| | PROTEINS | | |
| | 2013 U.S. Pat. No. 9,243,058 | | |
| | Armitage, Richard . . . | | |
| | BCMA antigen binding | | |
| | proteins | | |
| | 2013 U.S. Pat. No. 10,465,009 | | |
| | Armitage, Richard . . . | | |
| | BCMA antigen binding | | |
| | proteins | | |
| Autolus patent anti- | 2020 WO2020222021 | | Autolus |
| BCMA CAR | PULÉ, Martin, COR . . . | | |
| | ENGINEERED T-CELLS CO- | | |
| | EXPRESSING AN ANTI- | | |
| | BCMA CAR AND AN ANTI- | | |
| | ECTOENZYME ANTIBODY | | |
| | AND THEIR USE IN THE | | |
| | TREATMENT OF CANCER | | |
| | 2019 WO2020065330 | | |
| | PULÉ, Martin, COR . . . | | |
| | CHIMERIC ANTIGEN | | |
| | RECEPTOR | | |
| | 2019 US20220033509 | | |
| | Pule, Martin; Cor . . . | | |
| | CHIMERIC ANTIGEN | | |
| | RECEPTOR | | |
| | 2018 WO2018229492 | | |
| | KOKALAKI, Evangel . . . | | |
| | CHIMERIC ANTIGEN | | |
| | RECEPTOR | | |
| Beijing Immunochina | 2021 WO2022143870 LU, | | Beijing Immunochina |
| patent anti-BCMA | Xinan, HE, Ti . . . ANTIBODY | | |
| | THAT SPECIFICALLY BINDS | | |
| | TO BCMA AND | | |
| | APPLICATION THEREOF | | |
| belantamab mafodotin- | 2022 US20220411512 | 2022 NCT05461209 Phase | Glaxo Group Seattle |
| blmf | HOOS, Axel; KHAND . . . | 3 A Study of Comparing | Genetics |
| | COMBINATION | Talquetamab to | |
| | TREATMENT FOR CANCER | Belantamab Mafodotin in | |
| | 2022 US20230019140 | Participants With | |
| | KHANDEKAR, Sanjay . . . | Relapsed/Refractory | |
| | COMBINATION | Multiple Myeloma | |
| | TREATMENT FOR CANCER | 2022 NCT05393024 | |
| | WITH ANTI-BCMA | Observational Study in | |
| | BINDING PROTEIN AND | Patient With MMRR | |
| | PROTEOSOME INHIBITOR | Treated With Belantamab | |
| | 2021 WO2021195362 | Mafotidine on | |
| | CAMPBELL, Mary, V . . . | Monotherapy | |
| | METHODS OF TREATING | 2022 NCT05064358 Phase | |
| | MULTIPLE MYELOMA | 2 Study to Investigate | |
| | 2020 WO2021024133 | Alternative Dosing | |
| | KRANZ, James K., . . . | Regimens of Belantamab | |
| | BIOPHARMACUETICAL | Mafodotin in Participants | |
| | COMPOSITIONS AND | With Relapsed or | |
| | RELATED METHODS | Refractory Multiple | |
| | 2020 WO2020208572 | Myeloma | |
| | BISWAS, Swethajit . . . | 2021 NCT04876248 Phase | |
| | COMBINATION THERAPY | 2 Belantamab Mafodotin | |
| | WITH AN ANTI BCMA | and Lenalidomide for the | |
| | ANTIBODY AND A | Treatment of Multiple | |
| | GAMMA SECRETASE | Myeloma in Patients With | |
| | INHIBITOR | Minimal Residual Disease | |
| | 2020 US20220168417 | Positive After Stem Cell | |
| | BISWAS, Swethajit . . . | Transplant | |
| | COMBINATION THERAPY | 2021 NCT04892264 Phase | |
| | WITH AN ANTI BCMA | 1/Phase 2 Belantamab | |
| | ANTIBODY AND A | Mafodotin, Lenalidomide, | |
| | GAMMA SECRETASE | and Daratumumab for the | |
| | INHIBITOR | Treatment of Relapsed, | |
| | 2020 US20200197529 | Refractory, or Previously | |
| | Algate, Paul; Cle . . . | Untreated Multiple | |
| | ANTIGEN BINDING | Myeloma | |
| | PROTEINS | 2021 NCT04680468 Phase | |
| | 2020 U.S. Pat. No. 11,419,945 Algate, | 2 Study of Belantamab | |
| | Paul (Iss . . . Antigen | Mafodotin as Pre- and | |
| | binding proteins | Post-autologous Stem Cell | |
| | 2020 WO2020160375 | Transplant and | |
| | BISWAS, Swethajit . . . | Maintenance for Multiple | |
| | COMBINATION | Myeloma | |
| | TREATMENTS FOR | 2021 NCT04822337 Phase | |
| | CANCER COMPRISING | 1/Phase 2 A Phase I/II | |
| | BELANTAMAB | Study of Carfilzomib, | |
| | MAFODOTIN AND AN | Lenalidomide, | |
| | ANTI OX40 ANTIBODY | Dexamethasone and | |
| | AND USES AND | Belantamab Mafodotin in | |
| | METHODS THEREOF | Multiple Myeloma | |
| | 2020 WO2020160365 | 2021 NCT04617925 Phase | |
| | OPALINSKA, Joanna | 2 A Study of Belantamab | |
| | BELANTAMAB | Mafodotin in Patients With | |
| | MAFODOTIN IN | Relapsed or Refractory AL | |
| | COMBINATION WITH | Amyloidosis | |
| | PEMBROLIZUMAB FOR | 2020 NCT04484623 Phase | |
| | TREATING CANCER | 3 Belantamab Mafodotin | |
| | 2020 US20220096650 | Plus Pomalidomide and | |
| | OPALINSKA, Joanna; | Dexamethasone (Pd) | |
| | BELANTAMAB | Versus Bortezomib Plus Pd | |
| | MAFODOTIN IN | in Relapsed/Refractory | |
| | COMBINATION WITH | Multiple Myeloma | |
| | PEMBROLIZUMAB FOR | 2020 NCT04398680 Phase | |
| | TREATING CANCER | 1 A Study of Belantamab | |
| | 2020 US20220098303 | Mafodotin (GSK2857916) | |
| | Biswas, Swethajit . . . | in Multiple Myeloma | |
| | COMBINATION | Participants With Normal | |
| | TREATMENTS FOR | and Impaired Hepatic | |
| | CANCER COMPRISING | Function | |
| | BELANTAMAB | 2020 NCT04398745 Phase | |
| | MAFODOTIN AND AN | 1 A Study of Belantamab | |
| | ANTI OX40 ANTIBODY | Mafodotin (GSK2857916) | |
| | AND USES AND | in Multiple Myeloma | |
| | METHODS THEREOF | Participants With Normal | |
| | 2019 US20220003772 | and Impaired Renal | |
| | DETTMAN, Elisha J . . . | Function | |
| | METHODS OF TREATING | 2020 NCT04246047 Phase | |
| | CANCER | 3 Evaluation of Efficacy and | |
| | 2018 WO2019053613 | Safety of Belantamab | |
| | HOOS, Axel, KHAND . . . | Mafodotin, Bortezomib | |
| | COMBINATION | and Dexamethasone | |
| | TREATMENT FOR CANCER | Versus Daratumumab, | |
| | 2018 WO2019053612 | Bortezomib and | |
| | KHANDEKAR, Sanjay . . . | Dexamethasone in | |
| | COMBINATION | Participants With | |
| | TREATMENT FOR CANCER | Relapsed/Refractory | |
| | 2018 WO2019053611 | Multiple Myeloma | |
| | KHANDEKAR, Sanjay . . . | 2020 NCT04162210 Phase | |
| | COMBINATION | 3 Study of Single Agent | |
| | TREATMENT FOR CANCER | Belantamab Mafodotin | |
| | 2018 US20200255526 | Versus Pomalidomide Plus | |
| | HOOS, Axel; KHAND . . . | Low-dose Dexamethasone | |
| | COMBINATION | (Pom/Dex) in Participants | |
| | TREATMENT FOR CANCER | With Relapsed/Refractory | |
| | 2018 US20200254093 | Multiple Myeloma (RRMM) | |
| | KHANDEKAR, Sanjay . . . | 2019 NCT04177823 Phase | |
| | COMBINATION | 1 A Study of Belantamab | |
| | TREATMENT FOR CANCER | Mafodotin to Investigate | |
| | 2018 US20200270354 | Safety, Tolerability, | |
| | KHANDEKAR, Sanjay . . . | Pharmacokinetics, | |
| | COMBINATION | Immunogenicity and | |
| | TREATMENT FOR CANCER | Clinical Activity in | |
| | 2018 U.S. Pat. No. 11,401,334 | Participants With | |
| | Khandekar, Sanjay . . . | Relapsed/Refractory | |
| | Combination treatment | Multiple Myeloma (RRMM) | |
| | for cancer with anti- | 2019 NCT04091126 Phase | |
| | BCMA binding protein | 3 Bortezomib, | |
| | and proteosome inhibitor | Lenalidomide and | |
| | 2017 US20180147293 | Dexamethasone (VRd) | |
| | ALGATE, PAUL; CLE . . . | With Belantamab | |
| | ANTIGEN BINDING | Mafodotin Versus VRd | |
| | PROTEINS | Alone in Transplant | |
| | 2016 WO2017093942 | Ineligible Multiple | |
| | MAYES, Patrick A. . . . | Myeloma | |
| | COMBINATION | 2019 NCT04126200 Phase | |
| | TREATMENTS AND USES | 2 Platform Study of | |
| | AND METHODS THEREOF | Belantamab Mafodotin as | |
| | 2016 US20190256608 | Monotherapy and in | |
| | MAYES, Patrick A. . . . | Combination With Anti- | |
| | COMBINATION | cancer Treatments in | |
| | TREATMENTS AND USES | Participants With | |
| | AND METHODS THEREOF | Relapsed/Refractory | |
| | 2015 US20160193358 | Multiple Myeloma (RRMM) | |
| | Algate, Paul; Cle . . . | (DREAMM 5) | |
| | ANTIGEN BINDING | 2019 NCT03848845 Phase | |
| | PROTEINS | 2 Study Evaluating Safety, | |
| | 2013 US20130280280 | Tolerability and Clinical | |
| | ALGATE, PAUL; CLE . . . | Activity of GSK2857916 in | |
| | ANTIGEN BINDING | Combination With | |
| | PROTEINS | Pembrolizumab in Subjects | |
| | 2013 U.S. Pat. No. 9,273,141 Algate, | With Relapsed/Refractory | |
| | Paul; Cle . . . B cell | Multiple Myeloma (RRMM) | |
| | maturation antigen | 2019 NCT03828292 Phase | |
| | (BCMA) binding proteins | 1 An Open-label, Dose | |
| | 2012 WO2012163805 | Escalation Study in | |
| | ALGATE, Paul, CLE . . . | Japanese Subjects With | |
| | BCMA (CD269/TNFRSF17) - | Relapsed/Refractory | |
| | BINDING PROTEINS | Multiple Myeloma Who | |
| | 2012 US20140105915 | Have Failed Prior Anti | |
| | Algate, Paul; Cle . . . BCMA | Myeloma Treatments | |
| | (CD269/TNFRSF17) - | 2018 NCT03715478 Phase | |
| | BINDING PROTEINS | 1/Phase 2 Multi-Center | |
| | | Study of GSK2857916 in | |
| | | Combination With | |
| | | Pomalidomide and Dex | |
| | | 2018 NCT03763370 | |
| | | Compassionate Use | |
| | | Individual Request Program | |
| | | for GSK2857916 in Multiple | |
| | | Myeloma | |
| | | 2018 NCT03544281 Phase | |
| | | 2 To Evaluate Safety, | |
| | | Tolerability, and Clinical | |
| | | Activity of the Antibody- | |
| | | drug Conjugate, | |
| | | GSK2857916 Administered | |
| | | in Combination With | |
| | | Lenalidomide Plus | |
| | | Dexamethasone (Arm A), | |
| | | or in Combination With | |
| | | Bortezomib Plus | |
| | | Dexamethasone (Arm B) in | |
| | | Subjects With R . . . | |
| | | 2018 NCT03525678 Phase | |
| | | 2 A Study to Investigate the | |
| | | Efficacy and Safety of Two | |
| | | Doses of GSK2857916 in | |
| | | Subjects With Multiple | |
| | | Myeloma Who Have Failed | |
| | | Prior Treatment With an | |
| | | Anti-CD38 Antibody | |
| | | 2014 NCT02064387 Phase | |
| | | 1 Dose Escalation Study to | |
| | | Investigate the Safety, | |
| | | Pharmacokinetics, | |
| | | Pharmacodynamics, | |
| | | Immunogenicity and | |
| | | Clinical Activity of | |
| | | GSK2857916 | |
| | | 2022 McCurdy A, Visram A | |
| | | The Role of Belantamab | |
| | | Mafodotin, Selinexor, and | |
| | | Melflufen in Multiple | |
| | | Myeloma. | |
| | | 2022 Leong S, Lam HPJ, . . . | |
| | | Antibody drug conjugates | |
| | | for the treatment of | |
| | | Multiple Myeloma. | |
| | | 2022 Boytsov N, Stockl . . . | |
| | | MM-404 Real-World | |
| | | Belantamab Mafodotin | |
| | | Use: A US Retrospective | |
| | | Longitudinal Pharmacy and | |
| | | Medical Open-Source | |
| | | Claims Database | |
| | | Assessment. | |
| | | 2022 Quach H, Gironell . . . | |
| | | MM-459 Safety and Clinical | |
| | | Activity of Belantamab | |
| | | Mafodotin With | |
| | | Lenalidomide Plus | |
| | | Dexamethasone in Patients | |
| | | With Relapsed/Refractory | |
| | | Multiple Myeloma | |
| | | (RRMM): DREAMM-6 Arm- | |
| | | A Interim Analysis. | |
| | | 2022 Hultcrantz M, Kle . . . | |
| | | MM-470 Exploring | |
| | | Alternative Dosing | |
| | | Regimens of Single-Agent | |
| | | Belantamab Mafodotin on | |
| | | Safety and Efficacy in | |
| | | Patients With Relapsed or | |
| | | Refractory Multiple | |
| | | Myeloma (RRMM): | |
| | | DREAMM-14. | |
| | | 2022 Mohan M, Rein LE, . . . | |
| | | Corneal toxicity with | |
| | | belantamab mafodotin: | |
| | | Multi-institutional real-life | |
| | | experience. | |
| | | 2022 Atieh T, Atrash S . . . | |
| | | Belantamab in | |
| | | Combination with | |
| | | Dexamethasone in Patients | |
| | | with Triple-class | |
| | | Relapsed/Refractory | |
| | | Multiple Myeloma. | |
| | | 2022 Zhang Y, Godara A . . . | |
| | | Belantamab mafodotin in | |
| | | patients with | |
| | | relapsed/refractory AL | |
| | | amyloidosis with myeloma. | |
| | | 2022 Baines AC, Ershle . . . | |
| | | FDA Approval Summary: | |
| | | Belantamab Mafodotin for | |
| | | Patients with Relapsed or | |
| | | Refractory Multiple | |
| | | Myeloma. | |
| | | 2022 Aschauer J, Donne . . . | |
| | | Corneal Toxicity Associated | |
| | | With Belantamab | |
| | | Mafodotin Is Not | |
| | | Restricted to the | |
| | | Epithelium: Neuropathy | |
| | | Studied With Confocal | |
| | | Microscopy. | |
| | | 2022 Abeykoon JP, Vaxm . . . | |
| | | Impact of belantamab | |
| | | mafodotin-induced ocular | |
| | | toxicity on outcomes of | |
| | | patients with advanced | |
| | | multiple myeloma. | |
| | | 2022 Weisel K, Krishna . . . | |
| | | Matching-Adjusted Indirect | |
| | | Treatment Comparison to | |
| | | Assess the Comparative | |
| | | Efficacy of Ciltacabtagene | |
| | | Autoleucel in CARTITUDE-1 | |
| | | Versus Belantamab | |
| | | Mafodotin in DREAMM-2, | |
| | | Selinexor-Dexamethasone | |
| | | in STORM Part 2, and | |
| | | Melphalan Flufenamide- | |
| | | Dexamethasone in | |
| | | HORIZON for the | |
| | | Treatment of Patients With | |
| | | Triple-Class Exposed | |
| | | Relapsed or Refractory | |
| | | Multiple Myeloma. | |
| | | 2022 Gil-Sierra MD, Br . . . | |
| | | Belantamab mafodotin for | |
| | | relapsed or refractory | |
| | | multiple myeloma. | |
| | | 2021 Condorelli A, Gar . . . | |
| | | Belantamab Mafodotin and | |
| | | Relapsed/Refractory | |
| | | Multiple Myeloma: This Is | |
| | | Not Game Over. | |
| | | 2021 Marquant K, Quinq . . . | |
| | | Corneal in vivo confocal | |
| | | microscopy to detect | |
| | | belantamab mafodotin- | |
| | | induced ocular toxicity | |
| | | early and adjust the dose | |
| | | accordingly: a case report. | |
| | | 2021 Prawitz T, Popat . . . | |
| | | DREAMM-2: Indirect | |
| | | Comparisons of | |
| | | Belantamab Mafodotin vs. | |
| | | Selinexor + Dexamethasone | |
| | | and Standard of Care | |
| | | Treatments in | |
| | | Relapsed/Refractory | |
| | | Multiple Myeloma. | |
| | | 2021 Ferron-Brady G, R . . . | |
| | | Exposure-Response | |
| | | Analyses for Therapeutic | |
| | | Dose Selection of | |
| | | Belantamab Mafodotin in | |
| | | Patients with | |
| | | Relapsed/Refractory | |
| | | Multiple Myeloma. | |
| | | 2021 Lonial S, Lee HC, . . . | |
| | | Longer term outcomes | |
| | | with single-agent | |
| | | belantamab mafodotin in | |
| | | patients with relapsed or | |
| | | refractory multiple | |
| | | myeloma: 13-month | |
| | | follow-up from the pivotal | |
| | | DREAMM-2 study. | |
| | | 2021 Matsumiya W, Kara . . . | |
| | | Structural changes of | |
| | | corneal epithelium in | |
| | | belantamab-associated | |
| | | superficial keratopathy | |
| | | using anterior segment | |
| | | optical coherence | |
| | | tomography. | |
| | | 2021 Lonial S, Nooka A . . . | |
| | | Management of | |
| | | belantamab mafodotin- | |
| | | associated corneal events | |
| | | in patients with relapsed or | |
| | | refractory multiple | |
| | | myeloma (RRMM). | |
| | | 2021 Nooka AK, Weisel . . . | |
| | | Belantamab mafodotin in | |
| | | combination with novel | |
| | | agents in | |
| | | relapsed/refractory | |
| | | multiple myeloma: | |
| | | DREAMM-5 study design. | |
| | | 2020 Farooq AV, Degli . . . | |
| | | Correction to: Corneal | |
| | | Epithelial Findings in | |
| | | Patients with Multiple | |
| | | Myeloma Treated with | |
| | | Antibody-Drug Conjugate | |
| | | Belantamab Mafodotin in | |
| | | the Pivotal, Randomized, | |
| | | DREAMM-2 Study. | |
| | | 2020 Tzogani K, Pentti . . . | |
| | | The EMA Review of | |
| | | Belantamab Mafodotin | |
| | | (Blenrep) for the | |
| | | Treatment of Adult | |
| | | Patients with | |
| | | Relapsed/Refractory | |
| | | Multiple Myeloma. | |
| | | 2020 Richardson PG, Le . . . | |
| | | Single-agent belantamab | |
| | | mafodotin for | |
| | | relapsed/refractory | |
| | | multiple myeloma: analysis | |
| | | of the lyophilised | |
| | | presentation cohort from | |
| | | the pivotal DREAMM-2 | |
| | | study. | |
| | | 2020 Yu B, Jiang T, Liu D | |
| | | BCMA-targeted | |
| | | immunotherapy for | |
| | | multiple myeloma. | |
| | | 2020 Markham A | |
| | | Belantamab Mafodotin: | |
| | | First Approval. | |
| | | 2020 Sheikh S, Lebel E . . . | |
| | | Belantamab mafodotin in | |
| | | the treatment of relapsed | |
| | | or refractory multiple | |
| | | myeloma. | |
| | | 2020 Farooq AV, Degli . . . | |
| | | Corneal Epithelial Findings | |
| | | in Patients with Multiple | |
| | | Myeloma Treated with | |
| | | Antibody-Drug Conjugate | |
| | | Belantamab Mafodotin in | |
| | | the Pivotal, Randomized, | |
| | | DREAMM-2 Study. | |
| | | 2020 Popat R, Warcel D . . . | |
| | | Characterisation of | |
| | | response and corneal | |
| | | events with extended | |
| | | follow-up after belantamab | |
| | | mafodotin (GSK2857916) | |
| | | monotherapy for patients | |
| | | with relapsed multiple | |
| | | myeloma: a case series | |
| | | from the first-time-in- | |
| | | human clinical trial. | |
| | | 2019 Lonial S, Lee HC, . . . | |
| | | Belantamab mafodotin for | |
| | | relapsed or refractory | |
| | | multiple myeloma | |
| | | (DREAMM-2): a two-arm, | |
| | | randomised, open-label, | |
| | | phase 2 study. | |
| | | 2019 Trudel S, Lendvai . . . | |
| | | Antibody-drug conjugate, | |
| | | GSK2857916, in | |
| | | relapsed/refractory | |
| | | multiple myeloma: an | |
| | | update on safety and | |
| | | efficacy from dose | |
| | | expansion phase I study. | |
| | | 2016 Lee L, Bounds D, . . . | |
| | | Evaluation of B cell | |
| | | maturation antigen as a | |
| | | target for antibody drug | |
| | | conjugate mediated | |
| | | cytotoxicity in multiple | |
| | | myeloma. | |
| | | 2014 Tai YT, Mayes PA, . . . | |
| | | Novel afucosylated anti-B | |
| | | cell maturation antigen- | |
| | | monomethyl auristatin F | |
| | | antibody-drug conjugate | |
| | | (GSK2857916) induces | |
| | | potent and selective anti- | |
| | | multiple myeloma activity. | |
| | | AACR 2014 Novel anti-B | |
| | | cell maturation antigen- | |
| | | monomethyl auristatin F | |
| | | antibody-drug conjugate | |
| | | (GSK2857916) induces | |
| | | potent and selective anti- | |
| | | multiple myeloma activity | |
| | | via enhanced effector | |
| | | function and direct tumor | |
| | | cell killing Yu-Tzu Tai, | |
| | | Chira . . . | |
| | | ASH 2013 Evaluation Of | |
| | | Bcma As a Therapeutic | |
| | | Target In Multiple | |
| | | Myeloma Using An | |
| | | Antibody-Drug Conjugate | |
| | | Kwee L Yong, PhD, . . . | |
| | | ASH 2013 Novel Fc- | |
| | | Engineered Anti-B Cell | |
| | | Maturation Antigen- | |
| | | Monomethyl Auristatin F | |
| | | Antibody-Drug Conjugate | |
| | | (GSK2857916) Induces | |
| | | Potent and Selective Anti- | |
| | | Multiple Myeloma Activity | |
| | | Via Enhanced Effector | |
| | | Function and Direct Tumor | |
| | | Cell Killing Yu-Tzu Tai, PhD, | |
| | | . . . | |
| | | ASH 2017 Deep and | |
| | | Durable Responses in | |
| | | Patients (Pts) with | |
| | | Relapsed/Refractory | |
| | | Multiple Myeloma (MM) | |
| | | Treated with Monotherapy | |
| | | GSK2857916, an Antibody | |
| | | Drug Conjugate Against B- | |
| | | Cell Maturation Antigen | |
| | | (BCMA): Preliminary | |
| | | Results from Part 2 of | |
| | | Study BMA117159 | |
| BGI Shenzhen patent | 2019 WO2021031113 | | BGI Shenzhen |
| anti-BCMA | HOU, Yong, ZHAO, . . . | | Genoimmune |
| | ANTI-BCMA ANTIBODY | | |
| | AND USE THEREOF IN | | |
| | CAR-T FIELD | | |
| | 2018 WO2020073215 | | |
| | WANG, Meiniang, O . . . | | |
| | ANTI-BCMA SINGLE- | | |
| | CHAIN ANTIBODY SCFV, | | |
| | PREPARATION METHOD | | |
| | THEREFOR AND | | |
| | APPLICATION THEREOF | | |
| | 2018 US20210246220 | | |
| | WANG, Meiniang; O . . . | | |
| | Anti-BCMA single-chain | | |
| | antibody scFv and | | |
| | preparation method and | | |
| | application thereof | | |
| Biogen Idec patent anti- | 2021 US20220213208 | | Biogen |
| BCMA | Kalled, Susan L.; . . . ANTI- | | |
| | BCMA ANTIBODIES | | |
| | 2018 US20190161552 | | |
| | Kalled, Susan L.; . . . ANTI- | | |
| | BCMA ANTIBODIES | | |
| | 2018 U.S. Pat. No. 11,111,307 Kalled, | | |
| | Susan L. . . . Anti-BCMA | | |
| | antibodies | | |
| | 2016 US20170029518 | | |
| | Kalled, Susan L.; . . . ANTI- | | |
| | BCMA ANTIBODIES | | |
| | 2015 US20150125460 | | |
| | Kalled, Susan L.; . . . ANTI- | | |
| | BCMA ANTIBODIES | | |
| | 2010 U.S. Pat. No. 8,084,030 Kalled, | | |
| | Susan; Ra . . . Method of | | |
| | treating a patient having | | |
| | an autoimmune disorder | | |
| | by administering an | | |
| | antibody that binds | | |
| | human BCMA | | |
| | 2010 US20110110950 | | |
| | KALLED, Susan; Ra . . . | | |
| | Therapeutic regimens for | | |
| | BAFF antagonists | | |
| | 2010 WO2010104949 | | |
| | KALLED, Susan, L. . . . ANTI- | | |
| | BCMA ANTIBODIES | | |
| | 2010 U.S. Pat. No. 9,034,324 Kalled, | | |
| | Susan L.; . . . Anti-BCMA | | |
| | antibodies | | |
| | 2002 U.S. Pat. No. 7,083,785 | | |
| | Browning, Jeffrey . . . | | |
| | Methods of treatment by | | |
| | administering an anti- | | |
| | BCMA antibody | | |
| Biosion patent anti- | 2020 WO2021136308 | | Biosion |
| BCMA | CHEN, Mingjiu, TA . . . | | |
| | ANTIBODIES BINDING | | |
| | BCMA AND USES | | |
| | THEREOF | | |
| Care Thera patent anti- | 2019 WO2020087054 | | Care Thera |
| BCMA | WU, Lijun, GOLUBO . . . | | |
| | HUMANIZED BCMA-CAR- | | |
| | T CELLS | | |
| Caribou patent anti- | 2022 US20220220217 | | Caribou |
| BCMA | Wu, Lijun; Golubo . . . | | |
| | HUMANIZED BCMA | | |
| | ANTIBODY AND BCMA- | | |
| | CAR-T CELLS | | |
| | 2022 U.S. Pat. No. 11,472,884 Wu, | | |
| | Lijun; Golubo . . . | | |
| | Humanized BCMA | | |
| | antibody and BCMA-CAR- | | |
| | T cells | | |
| | 2021 US20220017633 | | |
| | WU, LIJUN; GOLUBO . . . | | |
| | HUMANIZED BCMA | | |
| | ANTIBODY AND BCMA- | | |
| | CAR-T CELLS | | |
| | 2021 U.S. Pat. No. 11,299,549 Wu, | | |
| | Lijun (Albany . . . | | |
| | Humanized BCMA | | |
| | antibody and BCMA-CAR- | | |
| | T cells | | |
| | 2021 US20210155704 | | |
| | Wu, Lijun; Golubo . . . | | |
| | HUMANIZED BCMA | | |
| | ANTIBODY AND BCMA- | | |
| | CAR-T CELLS | | |
| | 2021 U.S. Pat. No. 11,021,542 Wu, | | |
| | Lijun (Albany . . . | | |
| | Humanized BCMA | | |
| | antibody and BCMA-CAR- | | |
| | T cells | | |
| | 2020 US20200399387 | | |
| | Wu, LIJUN; Golubo . . . | | |
| | HUMANIZED BCMA | | |
| | ANTIBODY AND BCMA- | | |
| | CAR-T CELLS | | |
| | 2020 U.S. Pat. No. 10,927,182 Wu, | | |
| | Lijun (Albany . . . | | |
| | Humanized BCMA | | |
| | antibody and BCMA-CAR- | | |
| | T cells | | |
| | 2020 WO2020150339 | | |
| | WU, Lijun, GOLUBO . . . | | |
| | HUMANIZED BCMA | | |
| | ANTIBODY AND BCMA- | | |
| | CAR-T CELLS | | |
| Carsgen patent anti- | 2018 WO2018133877 | | Carsgen |
| BCMA | WANG, Peng, [symbol] , W . . . | | |
| | BCMA-TARGETING | | |
| | ANTIBODY AND USE | | |
| | THEREOF | | |
| | 2018 US20190359726 | | |
| | WANG, Peng; WANG, . . . | | |
| | BCMA-TARGETING | | |
| | ANTIBODY AND USE | | |
| | THEREOF | | |
| | 2018 U.S. Pat. No. 11,525,006 Wang, | | |
| | Peng; Wang, . . . BCMA- | | |
| | targeting antibody and | | |
| | use thereof | | |
| Cartesian patent anti- | 2020 WO2020190737 | | Cartesian |
| BCMA CAR | ZHANG, Yi, STEWAR . . . | | |
| | ANTI-BCMA CHIMERIC | | |
| | ANTIGEN RECEPTORS | | |
| Casebia patent anti- | 2019 WO2019210280 | | Casebia |
| BCMA CAR | COST, Gregory ANTI- | | |
| | BCMA CAR-T-CELLS FOR | | |
| | PLASMA CELL DEPLETION | | |
| CC-99712 | 2022 US20220324991 | 2019 NCT04036461 Phase | Celgene |
| | Abbasian, Mahan; . . . | 1 A Study of CC-99712, a | |
| | BCMA-BINDING | BCMA Antibody-Drug | |
| | ANTIBODIES AND USES | Conjugate, in Subjects With | |
| | THEREOF | Relapsed and Refractory | |
| | 2022 WO2022232488 | Multiple Myeloma | |
| | WU, Kaida | | |
| | COMBINATION | | |
| | THERAPIES USING AN | | |
| | ANTI-BCMA ANTIBODY | | |
| | DRUG CONJUGATE (ADC) | | |
| | IN COMBINATION WITH A | | |
| | GAMMA SECRETASE | | |
| | INHIBITOR (GSI) | | |
| | 2022 US20220401569 | | |
| | WU, Kaida | | |
| | COMBINATION | | |
| | THERAPIES USING AN | | |
| | ANTI-BCMA ANTIBODY | | |
| | DRUG CONJUGATE (ADC) | | |
| | IN COMBINATION WITH A | | |
| | GAMMA SECRETASE | | |
| | INHIBITOR (GSI) | | |
| | 2020 US20220323599 | | |
| | LEE, John; STAFFO . . . | | |
| | ANTI-BCMA ANTIBODY | | |
| | CONJUGATE, | | |
| | COMPOSITIONS | | |
| | COMPRISING THE SAME, | | |
| | AND METHODS OF | | |
| | MAKING AND USING THE | | |
| | SAME | | |
| | 2019 WO2019164891 | | |
| | ABBASIAN, Mahan, . . . | | |
| | BCMA-BINDING | | |
| | ANTIBODIES AND USES | | |
| | THEREOF | | |
| | 2019 US20200399386 | | |
| | Abbasian, Mahan; . . . | | |
| | BCMA-BINDING | | |
| | ANTIBODIES AND USES | | |
| | THEREOF | | |
| | 2019 U.S. Pat. No. 11,401,336 | | |
| | Abbasian, Mahan (. . . | | |
| | BCMA-binding antibodies | | |
| | and uses thereof | | |
| Cellectis patent anti- | 2019 US20190389959 | | Cellectis |
| BCMA CAR | GALETTO, Roman; BCMA | | |
| | (CD269) SPECIFIC | | |
| | CHIMERIC ANTIGEN | | |
| | RECEPTORS FOR CANCER | | |
| | IMMUNOTHERAPY | | |
| | 2019 U.S. Pat. No. 11,498,971 | | |
| | Galetto, Roman BCMA | | |
| | (CD269) specific chimeric | | |
| | antigen receptors for | | |
| | cancer immunotherapy | | |
| | 2015 US20170183418 | | |
| | GALLETTO, Roman; BCMA | | |
| | (CD269) SPECIFIC | | |
| | CHIMERIC ANTIGEN | | |
| | RECEPTORS FOR CANCER | | |
| | IMMUNOTHERAPY | | |
| | 2015 U.S. Pat. No. 10,316,101 | | |
| | Galetto, Roman (P . . . | | |
| | BCMA (CD269) specific | | |
| | chimeric antigen | | |
| | receptors for cancer | | |
| | immunotherapy | | |
| | 2015 WO2015158671 | | |
| | GALETTO, Roman BCMA | | |
| | (CD269) SPECIFIC | | |
| | CHIMERIC ANTIGEN | | |
| | RECEPTORS FOR CANCER | | |
| | IMMUNOTHERAPY | | |
| Cellular Biomed. patent | 2021 WO2022119923 | | Cellular Biomed. |
| anti-BCMA | YAO, Yihong, HUAN . . . | | |
| | BCMA-TARGETED | | |
| | CHIMERIC ANTIGEN | | |
| | RECEPTORS | | |
| | 2021 WO2021231213 | | |
| | HUANG, Jiaqi, YAO . . . | | |
| | ANTI-BCMA ANTIBODIES | | |
| | AND CHIMERIC ANTIGEN | | |
| | RECEPTORS | | |
| Celyad patent anti-BCMA | 2020 WO2020221873 | | Celyad |
| CAR | BORNSCHEIN, Simon . . . | | |
| | CAR T-CELLS TARGETING | | |
| | BCMA AND USES | | |
| | THEREOF | | |
| ciltacabtagene | 2022 WO2022248642 | 2020 NCT04181827 Phase | Janssen Biotech Nanjing |
| autoleucel | ADAMS III, Homer BCMA | 3 A Study Comparing JNJ- | Legend Bio |
| | AS A TARGET FOR T CELL | 68284528, a CAR-T Therapy | |
| | REDIRECTING | Directed Against B-cell | |
| | ANTIBODIES IN B CELL | Maturation Antigen | |
| | LYMPHOMAS | (BCMA), Versus | |
| | 2021 WO2022117068 | Pomalidomide, Bortezomib | |
| | SCHECTER, Jordan . . . | and Dexamethasone (PVd) | |
| | BCMA-TARGETED CAR-T | or Daratumumab, | |
| | CELL THERAPY FOR | Pomalidomide and | |
| | MULTIPLE MYELOMA | Dexamethasone (DPd) in | |
| | 2020 WO2022116086 | Participants With Relapsed | |
| | SCHECTER, Jordan . . . | and Lenalidomi . . . | |
| | BCMA-TARGETED CAR-T | 2019 NCT04133636 Phase | |
| | CELL THERAPY FOR | 2 A Study of JNJ-68284528, | |
| | MULTIPLE MYELOMA | a Chimeric Antigen | |
| | 2020 US20210128618 | Receptor T Cell (CAR-T) | |
| | ZUDAIRE UBANI, En . . . | Therapy Directed Against | |
| | BCMA-TARGETED CAR-T | B-cell Maturation Antigen | |
| | CELL THERAPY OF | (BCMA) in Participants | |
| | MULTIPLE MYELOMA | With Multiple Myeloma | |
| | 2020 WO2021091945 | 2018 NCT03548207 Phase | |
| | ZUDAIRE UBANI, En . . . | 1/Phase 2 A Study of JNJ- | |
| | BCMA-TARGETED CAR-T | 68284528, a Chimeric | |
| | CELL THERAPY OF | Antigen Receptor T Cell | |
| | MULTIPLE MYELOMA | (CAR-T) Therapy Directed | |
| | | Against B-Cell Maturation | |
| | | Antigen (BCMA) in | |
| | | Participants With Relapsed | |
| | | or Refractory Multiple | |
| | | Myeloma | |
| | | 2022 Usmani SZ, Martin . . . | |
| | | MM-181 CARTITUDE-1: | |
| | | Two-Year Post Last Patient | |
| | | in (LPI) Results From the | |
| | | Phase 1b/2 Study of | |
| | | Ciltacabtagene Autoleucel | |
| | | (Cilta-Cel), a B-Cell | |
| | | Maturation Antigen | |
| | | (BCMA)-Directed Chimeric | |
| | | Antigen Receptor T (CAR-T) | |
| | | Cell Therapy, in Patients | |
| | | With Relapsed/Refractory | |
| | | Multiple Myeloma | |
| | | (RRMM). | |
| | | 2021 Weisel K, Martin . . . | |
| | | Comparative Efficacy of | |
| | | Ciltacabtagene Autoleucel | |
| | | in CARTITUDE-1 vs | |
| | | Physician's Choice of | |
| | | Therapy in the Long-Term | |
| | | Follow-Up of POLLUX, | |
| | | CASTOR, and EQUULEUS | |
| | | Clinical Trials for the | |
| | | Treatment of Patients with | |
| | | Relapsed or Refractory | |
| | | Multiple Myeloma. | |
| | | 2021 Martin T, Usmani . . . | |
| | | Matching-adjusted indirect | |
| | | comparison of efficacy | |
| | | outcomes for | |
| | | ciltacabtagene autoleucel | |
| | | in CARTITUDE-1 versus | |
| | | idecabtagene vicleucel in | |
| | | KarMMa for the treatment | |
| | | of patients with relapsed or | |
| | | refractory multiple | |
| | | myeloma. | |
| | | 2021 Berdeja JG, Maddu . . . | |
| | | Ciltacabtagene autoleucel, | |
| | | a B-cell maturation | |
| | | antigen-directed chimeric | |
| | | antigen receptor T-cell | |
| | | therapy in patients with | |
| | | relapsed or refractory | |
| | | multiple myeloma | |
| | | (CARTITUDE-1): a phase | |
| | | 1b/2 open-label study. | |
| Crispr Thera patent anti- | 2021 WO2022043861 | | Crispr Thera |
| BCMA CAR | DAR, Henia, KUMAR . . . | | |
| | ANTI-IDIOTYPE | | |
| | ANTIBODIES TARGETING | | |
| | ANTI-BCMA CHIMERIC | | |
| | ANTIGEN RECEPTOR | | |
| CT053 | | 2019 NCT03975907 Phase | Carsgen |
| | | 1/Phase 2 Clinical Trial to | |
| | | Evaluate BCMA Car-T | |
| | | (CT053) in Patients With | |
| | | Relapsed and/or Refractory | |
| | | Multiple Myeloma | |
| Cure Genetics patent | 2020 WO2021057866 | | Cure Genetics |
| anti-BCMA CAR | WANG, Wenbo, FENG . . . | | |
| | SINGLE DOMAIN | | |
| | ANTIBODY AND | | |
| | CHIMERIC ANTIGEN | | |
| | RECEPTOR COMPRISING | | |
| | ANTIBODY STRUCTURE | | |
| Curocell patent anti- | 2021 WO2021182929 | | Curocell |
| BCMA CAR | SHIM, Hyun Bo, KI . . . | | |
| | BCMA-SPECIFIC | | |
| | ANTIBODY AND | | |
| | CHIMERIC ANTIGEN | | |
| | RECEPTOR | | |
| Eisai patent anti-BCMA | 2021 WO2021248005 | | Eisai |
| | HENRY, Ryan, SAMA . . . | | |
| | ANTI-BCMA ANTIBODY- | | |
| | DRUG CONJUGATES AND | | |
| | METHODS OF USE | | |
| | 2021 US20220081486 | | |
| | Henry, Ryan; Sama . . . | | |
| | ANTI-BCMA ANTIBODY- | | |
| | DRUG CONJUGATES AND | | |
| | METHODS OF USE | | |
| Elstar patent anti-BCMA/ | 2018 WO2019035938 | | Elstar |
| X | LOEW, Andreas, VA . . . | | |
| | MULTISPECIFIC | | |
| | MOLECULES THAT BIND | | |
| | TO BCMA AND USES | | |
| | THEREOF | | |
| Engmab patent anti- | 2020 US20210070873 | | Engmab |
| BCMA | VU, Minh Diem; St . . . | | |
| | METHOD FOR THE | | |
| | SELECTION OF | | |
| | ANTIBODIES AGAINST | | |
| | BCMA | | |
| | 2020 US20200283545 Vu, | | |
| | Minh Diem; St . . . | | |
| | MONOCLONAL | | |
| | ANTIBODIES AGAINST | | |
| | BCMA | | |
| | 2018 US20180222991 | | |
| | VU, Minh Diem; ST . . . | | |
| | METHOD FOR THE | | |
| | SELECTION OF | | |
| | ANTIBODIES AGAINST | | |
| | BCMA | | |
| | 2018 U.S. Pat. No. 10,851,171 Vu, | | |
| | Minh Diem (Wo . . . | | |
| | Method for the selection | | |
| | of antibodies against | | |
| | BCMA | | |
| | 2016 WO2017021450 VU, | | |
| | Minh, Diem, S . . . | | |
| | MONOCLONAL | | |
| | ANTIBODIES AGAINST | | |
| | BCMA | | |
| | 2016 US20190352427 Vu, | | |
| | Minh Diem; St . . . | | |
| | MONOCLONAL | | |
| | ANTIBODIES AGAINST | | |
| | BCMA | | |
| | 2016 U.S. Pat. No. 10,683,369 Vu, | | |
| | Minh Diem (Wo . . . | | |
| | Monoclonal antibodies | | |
| | against BCMA | | |
| | 2014 WO2014122143 VU, | | |
| | Minh Diem, ST . . . | | |
| | METHOD FOR THE | | |
| | SELECTION OF | | |
| | ANTIBODIES AGAINST | | |
| | BCMA | | |
| | 2014 U.S. Pat. No. 9,963,513 Vu, | | |
| | Minh Diem; St . . . Method | | |
| | for the selection of | | |
| | antibodies against BCMA | | |
| Fosun Kite patent anti- | 2021 WO2021213478 | | Fosun Kite |
| BCMA | ZHANG, Lei, XU, M . . . | | |
| | ANTI-HUMAN B7-H3 | | |
| | MONOCLONAL | | |
| | ANTIBODY AND | | |
| | APPLICATION THEREOF | | |
| Fred Hutchinson CRC | 2018 WO2018151836 | | FHCRC |
| patent anti-BCMA CAR | RIDDELL, Stanley, . . . | | |
| | COMBINATION | | |
| | THERAPIES FOR | | |
| | TREATMENT OF BCMA- | | |
| | RELATED CANCERS AND | | |
| | AUTOIMMUNE | | |
| | DISORDERS | | |
| | 2018 US20190359727 | | |
| | RIDDELL, Stanley . . . | | |
| | COMBINATION | | |
| | THERAPIES FOR | | |
| | TREATMENT OF BCMA- | | |
| | RELATED CANCERS AND | | |
| | AUTOIMMUNE | | |
| | DISORDERS | | |
| Genbase patent anti- | 2019 WO2021051390 | | Genbase |
| BCMA | DU, Liang, MOU, N . . . | | |
| | BCMA-TARGETED | | |
| | ANTIBODY AND | | |
| | CHIMERIC ANTIGEN | | |
| | RECEPTOR | | |
| | 2019 US20220331363 Du, | | |
| | Liang; Mou, N . . . BCMA- | | |
| | TARGETED ANTIBODY | | |
| | AND CHIMERIC ANTIGEN | | |
| | RECEPTOR | | |
| Gracell Bio patent anti- | 2020 WO2020224606 | | Gracell Bio |
| BCMA CAR | ZHANG, Hua, SHI, . . . | | |
| | ENGINEERED IMMUNE | | |
| | CELL TARGETING BCMA | | |
| | AND USE THEREOF | | |
| | 2020 WO2020224605 | | |
| | ZHANG, Hua, SHEN, . . . | | |
| | BCMA-TARGETING | | |
| | ENGINEERED IMMUNE | | |
| | CELL AND USE THEREOF | | |
| | 2020 US20220049004 | | |
| | ZHANG, Hua; SHI, . . . | | |
| | ENGINEERED IMMUNE | | |
| | CELLS TARGETING BCMA | | |
| | AND THEIR USES | | |
| | THEREOF | | |
| | 2020 US20220202864 | | |
| | ZHANG, Hua; SHEN, . . . | | |
| | BCMA-TARGETING | | |
| | ENGINEERED IMMUNE | | |
| | CELL AND USE THEREOF | | |
| Hangzhou Sumgen | 2020 WO2021032130 LV, | | Hangzhou Sumgen |
| Biotech patent anti- | Ming, DING, X . . . BCMA | | Biotech |
| BCMA | ANTIBODY | | |
| | 2020 US20220306757 LV, | | |
| | Ming; DING, X . . . BCMA | | |
| | ANTIBODY | | |
| Hansoh patent anti- | 2022 WO2022161385 | | Hansoh Pharma |
| BCMA | HUA, Haiqing, MAO . . . | | |
| | ANTIBODY-DRUG | | |
| | CONJUGATE AND | | |
| | MEDICAL USE THEREOF | | |
| | 2021 WO2021190564 | | |
| | HUA, Haiqing, BAO . . . | | |
| | ANTIBODY-DRUG | | |
| | CONJUGATE AND | | |
| | MEDICAL USE THEREOF | | |
| | 2020 WO2021018168 | | |
| | HUA, Haiqing, BAO . . . | | |
| | ANTI-BCMA ANTIBODY, | | |
| | ANTIGEN-BINDING | | |
| | FRAGMENT THEREOF | | |
| | AND MEDICAL USE | | |
| | THEREOF | | |
| | 2020 US20220251228 | | |
| | HUA, Haiqing; BAO . . . | | |
| | ANTI-BCMA ANTIBODY, | | |
| | ANTIGEN-BINDING | | |
| | FRAGMENT THEREOF | | |
| | AND MEDICAL USE | | |
| | THEREOF | | |
| Harbour Biomed patent | 2021 WO2021147941 | | Harbour Biomed Ltd. |
| anti-BCMA | WANG, Zheng, HE, . . . | | |
| | BCMA-BINDING PROTEIN, | | |
| | PREPARATION METHOD | | |
| | THEREFOR, AND | | |
| | APPLICATION THEREOF | | |
| HDP-101 | 2017 US20190328899 | 2020 Figueroa-Vazquez . . . | Heidelberg Pharma |
| | Hechler, Torsten; . . . | HDP-101, anti-BCMA | |
| | AMANITIN ANTIBODY | antibody-drug conjugate, | |
| | CONJUGATES | safely delivers amanitin to | |
| | | induce cell death in | |
| | | proliferating and resting | |
| | | multiple myeloma cells. | |
| | | AACR 2017 Preclinical | |
| | | evaluation of HDP-101, an | |
| | | anti-BCMA antibody-drug | |
| | | conjugate Torsten Hechler, | |
| | | . . . | |
| | | ASCO 2018 HDP-101: | |
| | | Preclinical evaluation of a | |
| | | novel anti-BCMA antibody | |
| | | drug conjugates in multiple | |
| | | myeloma Andreas Pahl, | |
| | | Jon . . . | |
| | | ASH 2017 Preclinical | |
| | | Evaluation of Hdp-101, a | |
| | | Novel Anti-Bcma Antibody- | |
| | | Drug Conjugate, in Multiple | |
| | | Myeloma Jonathan Ko, | |
| | | Chri . . . | |
| Hrain Bio patent anti- | 2018 WO2020061796 | | Hrain Bio |
| BCMA CAR | LIU, Yarong, HE, . . . BCMA- | | |
| | AND-CD19-TARGETING | | |
| | CHIMERIC ANTIGEN | | |
| | RECEPTOR AND USES | | |
| | THEREOF | | |
| | 2018 WO2020034081 | | |
| | LIU, Yarong, HE, . . . BCMA- | | |
| | TARGETING CHIMERIC | | |
| | ANTIGEN RECEPTOR AND | | |
| | USES THEREOF | | |
| | 2018 US20210221903 | | |
| | LIU, Yarong; HE, . . . BCMA- | | |
| | TARGETING CHIMERIC | | |
| | ANTIGEN RECEPTOR AND | | |
| | USES THEREOF | | |
| Hunan Acemab patent | 2021 WO2021160133 LI, | | Hunan Acemab |
| anti-BCMA | Jianliang, WO . . . ANTI- | | |
| | BCMA ANTIBODY, | | |
| | PHARMACEUTICAL | | |
| | COMPOSITION OF SAME, | | |
| | AND APPLICATIONS | | |
| | THEREOF | | |
| idecabtagene vicleucel | 2021 WO2022046730 | 2016 NCT02658929 Phase | Bluebird BMS Celgene |
| | CONTRASTANO, Shan . . . | 1 Study of bb2121 in | |
| | BCMA CHIMERIC | Multiple Myeloma | |
| | ANTIGEN RECEPTORS | 2021 Madduri D, Parekh . . . | |
| | 2020 WO2021127007 | Anti-BCMA CAR T | |
| | FRIEDMAN, Kevin, . . . | administration in a | |
| | ANTI-BCMA CAR | relapsed and refractory | |
| | ANTIBODIES, | multiple myeloma patient | |
| | CONJUGATES, AND | after COVID-19 infection: a | |
| | METHODS OF USE | case report. | |
| | 2020 WO2021091978 | ASH 2015 Novel and Highly | |
| | CAMPBELL, Timothy . . . | Potent CAR T Cell Drug | |
| | USES OF ANTI-BCMA | Product for Treatment of | |
| | CHIMERIC ANTIGEN | BCMA-Expressing | |
| | RECEPTORS | Hematological Malignances | |
| | 2020 WO2020206061 | Alena A. Chekmaso . . . | |
| | FRIEDMAN, Kevin, . . . | ASH 2015 Manufacturing | |
| | MANUFACTURING ANTI- | an Enhanced CAR T Cell | |
| | BCMA CAR T CELLS | Product By Inhibition of the | |
| | 2019 WO2020014333 | PI3K/Akt Pathway During T | |
| | HEGE, Kristen, PA . . . USES | Cell Expansion Results in | |
| | OF ANTI-BCMA CHIMERIC | Improved In Vivo Efficacy | |
| | ANTIGEN RECEPTORS | of Anti-BCMA CAR T Cells | |
| | 2019 WO2019241686 | Molly R. Perkins, . . . | |
| | FRIEDMAN, Kevin, . . . | ASH 2015 Characterization | |
| | ANTI-BCMA CAR | of Lentiviral Vector Derived | |
| | ANTIBODIES, | Anti-Bcma CAR T Cells | |
| | CONJUGATES, AND | Reveals Key Parameters for | |
| | METHODS OF USE | Robust Manufacturing of | |
| | 2019 US20210261689 | Cell-Based Gene Therapies | |
| | FRIEDMAN, Kevin; . . . | for Multiple Myeloma | |
| | ANTI-BCMA CAR | Graham Lilley, M. . . . | |
| | ANTIBODIES, | | |
| | CONJUGATES, AND | | |
| | METHODS OF USE | | |
| | 2017 WO2018085690 | | |
| | QUIGLEY, Travis, . . . ANTI- | | |
| | BCMA CAR T CELL | | |
| | COMPOSITIONS | | |
| | 2015 WO2016094304 | | |
| | MORGAN, Richard, . . . | | |
| | BCMA CHIMERIC | | |
| | ANTIGEN RECEPTORS | | |
| Innovent patent anti- | 2019 WO2019149269 LI, | | Innovent |
| BCMA | Zhiyuan, ZHAI . . . FULLY | | |
| | HUMAN ANTI-B CELL | | |
| | MATURATION ANTIGEN | | |
| | (BCMA) SINGLE CHAIN | | |
| | VARIABLE FRAGMENT, | | |
| | AND APPLICATION | | |
| | THEREOF | | |
| | 2019 US20200339699 LI, | | |
| | Zhiyuan; ZHAI . . . FULLY | | |
| | HUMANIZED ANTI-B CELL | | |
| | MATURATION ANTIGEN | | |
| | (BCMA) SINGLE-CHAIN | | |
| | ANTIBODY AND USE | | |
| | THEREOF | | |
| Juno patent anti-BCMA | 2022 WO2022221726 | | Juno Sloan-Kettering |
| CAR | RYTLEWSKI, Julie Ann | | |
| | COMBINATION | | |
| | THERAPIES WITH BCMA- | | |
| | DIRECTED T CELL | | |
| | THERAPY | | |
| | 2021 US20210324100 | | |
| | SATHER, Blythe D. . . . | | |
| | CHIMERIC ANTIGEN | | |
| | RECEPTORS SPECIFIC FOR | | |
| | B-CELL MATURATION | | |
| | ANTIGEN AND ENCODING | | |
| | POLYNUCLEOTIDES | | |
| | 2021 WO2021222330 | | |
| | PORTS, Michael | | |
| | COMBINATION OF BCMA- | | |
| | DIRECTED T CELL | | |
| | THERAPY AND AN | | |
| | IMMUNOMODULATORY | | |
| | COMPOUND | | |
| | 2021 WO2021207689 | | |
| | STIRNER, Mariana, . . . | | |
| | METHODS AND USES | | |
| | RELATED TO CELL | | |
| | THERAPY ENGINEERED | | |
| | WITH A CHIMERIC | | |
| | ANTIGEN RECEPTOR | | |
| | TARGETING B-CELL | | |
| | MATURATION ANTIGEN | | |
| | 2020 US20230028050 | | |
| | HAUSKINS, Collin; . . . ANTI- | | |
| | IDIOTYPIC ANTIBODIES | | |
| | TO BCMA-TARGETED | | |
| | BINDING DOMAINS AND | | |
| | RELATED COMPOSITIONS | | |
| | AND METHODS | | |
| | 2019 WO2020092848 | | |
| | SATHER, Blythe D. . . . | | |
| | METHODS FOR | | |
| | TREATMENT USING | | |
| | CHIMERIC ANTIGEN | | |
| | RECEPTORS SPECIFIC FOR | | |
| | B-CELL MATURATION | | |
| | ANTIGEN | | |
| | 2018 WO2019090003 | | |
| | SATHER, Blythe D. . . . | | |
| | CHIMERIC ANTIGEN | | |
| | RECEPTORS SPECIFIC FOR | | |
| | B-CELL MATURATION | | |
| | ANTIGEN (BCMA) | | |
| | 2018 US20190161553 | | |
| | SATHER, Blythe D. . . . | | |
| | CHIMERIC ANTIGEN | | |
| | RECEPTORS SPECIFIC FOR | | |
| | B-CELL MATURATION | | |
| | ANTIGEN AND ENCODING | | |
| | POLYNUCLEOTIDES | | |
| | 2018 US20200392236 | | |
| | SATHER, Blythe D. . . . | | |
| | ANTIBODIES AND | | |
| | CHIMERIC ANTIGEN | | |
| | RECEPTORS SPECIFIC FOR | | |
| | B-CELL MATURATION | | |
| | ANTIGEN | | |
| | 2018 U.S. Pat. No. 11,066,475 Sather, | | |
| | Blythe D. . . . Chimeric | | |
| | antigen receptors specific | | |
| | for B-cell maturation | | |
| | antigen and encoding | | |
| | polynucleotides | | |
| Kite patent anti-BCMA | 2019 US20200109209 | | Kite |
| CAR | WILTZIUS, Jed; AL . . . | | |
| | BCMA BINDING | | |
| | MOLECULES AND | | |
| | METHODS OF USE | | |
| | THEREOF | | |
| | 2019 U.S. Pat. No. 11,505,613 | | |
| | Wiltzius, Jed; Al . . . BCMA | | |
| | binding molecules and | | |
| | methods of use thereof | | |
| | 2019 US20200377609 | | |
| | Sievers, Stuart; . . . ANTI- | | |
| | IDIOTYPIC ANTIBODIES | | |
| | DIRECTED TO THE | | |
| | ANTIGEN-BINDING | | |
| | PORTION OF AN BCMA- | | |
| | BINDING MOLECULE | | |
| | 2017 US20170283504 | | |
| | WILTZIUS, Jed; AL . . . | | |
| | BCMA BINDING | | |
| | MOLECULES AND | | |
| | METHODS OF USE | | |
| | THEREOF | | |
| | 2017 WO2017173349 | | |
| | WILTZIUS, Jed, AL . . . | | |
| | BCMA BINDING | | |
| | MOLECULES AND | | |
| | METHODS OF USE | | |
| | THEREOF | | |
| | 2017 WO2017173256 | | |
| | WILTZIUS, Jed CHIMERIC | | |
| | ANTIGEN AND T CELL | | |
| | RECEPTORS AND | | |
| | METHODS OF USE | | |
| | 2017 U.S. Pat. No. 10,689,450 | | |
| | Wiltzius, Jed (Wo . . . BCMA | | |
| | binding molecules and | | |
| | methods of use thereof | | |
| LCAR-B38M | 2021 US20220127371 | 2019 NCT03758417 Phase | Nanjing Legend Bio |
| | FAN, Xiaohu; ZHUA . . . | 2 A Study of LCAR-B38M | |
| | CHIMERIC ANTIGEN | CAR-T Cells, a Chimeric | |
| | RECEPTORS TARGETING | Antigen Receptor T-cell | |
| | BCMA AND METHODS OF | (CAR-T) Therapy Directed | |
| | USE THEREOF | Against B-cell Maturation | |
| | 2021 US20210163615 | Antigen (BCMA) in Chinese | |
| | FAN, Xiaohu; ZHUA . . . | Participants With Relapsed | |
| | CHIMERIC ANTIGEN | or Refractory Multiple | |
| | RECEPTORS TARGETING | Myeloma | |
| | BCMA AND METHODS OF | 2018 NCT03674463 Phase | |
| | USE THEREOF | 1 LCAR-B4822M-02 Cells in | |
| | 2021 U.S. Pat. No. 11,186,647 Fan, | Treating | |
| | Xiaohu (Edmo . . . Chimeric | Relapsed/Refractory (R/R) | |
| | antigen receptors | Multiple Myeloma | |
| | targeting BCMA and | 2015 NCT03090659 Phase | |
| | methods of use thereof | 1/Phase 2 LCAR-B38M-02 | |
| | 2020 WO2021121228 | Cells in Treating | |
| | FAN, Xiaohu, ZHUA . . . | Relapsed/Refractory (R/R) | |
| | SINGLE DOMAIN | Multiple Myeloma | |
| | ANTIBODIES AND | | |
| | CHIMERIC ANTIGEN | | |
| | RECEPTORS TARGETING | | |
| | BCMA AND METHODS OF | | |
| | USE THEREOF | | |
| | 2017 WO2018028647 | | |
| | FAN, Xiaohu, ZHUA .. | | |
| | CHIMERIC ANTIGEN | | |
| | RECEPTORS TARGETING | | |
| | BCMA AND METHODS OF | | |
| | USE THEREOF | | |
| | 2017 US20200078399 | | |
| | FAN, Xiaohu; ZHUA . . . | | |
| | CHIMERIC ANTIGEN | | |
| | RECEPTORS TARGETING | | |
| | BCMA AND METHODS OF | | |
| | USE THEREOF | | |
| | 2017 U.S. Pat. No. 11,535,677 Fan, | | |
| | Xiaohu; Zhua . . . Chimeric | | |
| | antigen receptors | | |
| | targeting BCMA and | | |
| | methods of use thereof | | |
| Lentigen patent anti- | 2021 US20210361709 | | Lentigen |
| BCMA CAR | Schneider, Dina; . . . | | |
| | COMPOSITIONS AND | | |
| | METHODS FOR TREATING | | |
| | CANCER WITH ANTI- | | |
| | BCMA IMMUNOTHERAPY | | |
| | 2020 US20200289572 | | |
| | Schneider, Dina; . . . | | |
| | COMPOSITIONS AND | | |
| | METHODS FOR TREATING | | |
| | CANCER WITH ANTI- | | |
| | BCMA IMMUNOTHERAPY | | |
| | 2020 WO2020243546 | | |
| | SCHNEIDER, Dina, . . . | | |
| | COMPOSITIONS AND | | |
| | METHODS FOR TREATING | | |
| | CANCER WITH ANTI- | | |
| | BCMA IMMUNOTHERAPY | | |
| | 2020 U.S. Pat. No. 11,052,112 | | |
| | Schneider, Dina (. . . | | |
| | Compositions and | | |
| | methods for treating | | |
| | cancer with anti-BCMA | | |
| | immunotherapy | | |
| Max Delbruck Center | 2020 US20210079108 | | Max Delbruck Center |
| patent anti-BCMA | Oden, Felix; Mari . . . | | |
| | HUMANIZED ANTIBODIES | | |
| | AGAINST CD269 (BCMA) | | |
| | 2020 US20200369778 | | |
| | LIPP, Martin; ODE . . . | | |
| | ANTIBODY THAT BINDS | | |
| | CD269 (BCMA) SUITABLE | | |
| | FOR USE IN THE | | |
| | TREATMENT OF PLASMA | | |
| | CELL DISEASES SUCH AS | | |
| | MULTIPLE MYELOMA | | |
| | AND AUTOIMMUNE | | |
| | DISEASES | | |
| | 2018 US20190106499 | | |
| | Lipp, Martin; Ode . . . | | |
| | ANTIBODY THAT BINDS | | |
| | CD269 (BCMA) SUITABLE | | |
| | FOR USE IN THE | | |
| | TREATMENT OF PLASMA | | |
| | CELL DISEASES SUCH AS | | |
| | MULTIPLE MYELOMA | | |
| | AND AUTOIMMUNE | | |
| | DISEASES | | |
| | 2018 U.S. Pat. No. 10,745,486 Lipp, | | |
| | Martin (Gli . . . Antibody | | |
| | that binds CD269 (BCMA) | | |
| | suitable for use in the | | |
| | treatment of plasma cell | | |
| | diseases such as multiple | | |
| | myeloma and | | |
| | autoimmune diseases | | |
| | 2018 US20190112382 | | |
| | Oden, Felix; Mari . . . | | |
| | HUMANIZED ANTIBODIES | | |
| | AGAINST CD269 (BCMA) | | |
| | 2018 U.S. Pat. No. 10,851,172 Oden, | | |
| | Felix (Berl . . . Humanized | | |
| | antibodies against CD269 | | |
| | (BCMA) | | |
| | 2017 WO2017211900 | | |
| | REHM, Armin, HÖPK . . . | | |
| | CHIMERIC ANTIGEN | | |
| | RECEPTOR AND CAR-T | | |
| | CELLS THAT BIND BCMA | | |
| | 2017 US20190307797 | | |
| | Rehm, Armin; Hopk . . . | | |
| | CHIMERIC ANTIGEN | | |
| | RECEPTOR AND CAR-T | | |
| | CELLS THAT BIND BCMA | | |
| | 2015 WO2015166073 | | |
| | ODEN, Felix, MARI . . . | | |
| | HUMANIZED ANTIBODIES | | |
| | AGAINST CD269 (BCMA) | | |
| | 2015 US20170166649 | | |
| | Oden, Felix; Mari . . . | | |
| | HUMANIZED ANTIBODIES | | |
| | AGAINST CD269 (BCMA | | |
| | 2015 U.S. Pat. No. 10,144,782 Oden, | | |
| | Felix (Berl . . . Humanized | | |
| | antibodies against CD269 | | |
| | (BCMA) | | |
| | 2013 WO2014068079 | | |
| | LIPP, Martin, ODE . . . AN | | |
| | ANTIBODY THAT BINDS | | |
| | CD269 (BCMA) SUITABLE | | |
| | FOR USE IN THE | | |
| | TREATMENT OF PLASMA | | |
| | CELL DISEASES SUCH AS | | |
| | MULTIPLE MYELOMA | | |
| | AND AUTOIMMUNE | | |
| | DISEASES | | |
| | 2013 US20150284467 | | |
| | Lipp, Martin; Ode . . . | | |
| | ANTIBODY THAT BINDS | | |
| | CD269 (BCMA) SUITABLE | | |
| | FOR USE IN THE | | |
| | TREATMENT OF PLASMA | | |
| | CELL DISEASES SUCH AS | | |
| | MULTIPLE MYELOMA | | |
| | AND AUTOIMMUNE | | |
| | DISEASES | | |
| | 2013 U.S. Pat. No. 10,189,906 Lipp, | | |
| | Martin (Gli . . . Antibody | | |
| | that binds CD269 (BCMA) | | |
| | suitable for use in the | | |
| | treatment of plasma cell | | |
| | diseases such as multiple | | |
| | myeloma and | | |
| | autoimmune diseases | | |
| McMaster U. anti-BCMA | 2022 WO2022266778 | | McMaster U. |
| | BRAMSON, Jonathan . . . | | |
| | BCMA T CELL-ANTIGEN | | |
| | COUPLERS AND USES | | |
| | THEREOF | | |
| MEDI2228 | 2021 US20210214460 | 2018 NCT03489525 Phase | Medimmune |
| | KINNEER, Krista; . . . BCMA | 1 MEDI2228 in Subjects | |
| | Monoclonal Antibody- | With Relapsed/Refractory | |
| | Drug Conjugate | Multiple Myeloma | |
| | 2020 US20220218835 | 2021 Xing L, Wang S, L . . . | |
| | KINNEER, Krista; . . . | BCMA-specific ADC | |
| | COMBINATION THERAPY | MEDI2228 and | |
| | 2018 US20190040152 | Daratumumab induce | |
| | KINNEER, Krista; . . . BCMA | synergistic myeloma | |
| | Monoclonal Antibody- | cytotoxicity via IFN-driven | |
| | Drug Conjugate | immune responses and | |
| | 2018 WO2019025983 | enhanced CD38 expression. | |
| | KINNEER, Krista, . . . BCMA | 2020 Xing L, Lin L, Yu . . . A | |
| | MONOCLONAL | novel BCMA PBD-ADC with | |
| | ANTIBODY-DRUG | ATM/ATR/WEE1 inhibitors | |
| | CONJUGATE | or bortezomib induce | |
| | 2018 U.S. Pat. No. 10,988,546 | synergistic lethality in | |
| | Kinneer, Krista (. . . BCMA | multiple myeloma. | |
| | monoclonal antibody- | 2018 Kinneer K, Flynn . . . | |
| | drug conjugate | Preclinical assessment of | |
| | | an antibody-PBD conjugate | |
| | | that targets BCMA on | |
| | | multiple myeloma and | |
| | | myeloma progenitor cells. | |
| | | 2018 Kinneer K, Meekin . . . | |
| | | SLC46A3 as a potential | |
| | | predictive biomarker for | |
| | | antibody-drug conjugates | |
| | | bearing non-cleavable | |
| | | linked maytansinoid and | |
| | | pyrrolobenzodiazepine | |
| | | warheads. | |
| | | ASH 2017 Preclinical | |
| | | Evaluation of MEDI2228, a | |
| | | BCMA-Targeting | |
| | | Pyrrolobenzodiazepine- | |
| | | Linked Antibody Drug | |
| | | Conjugate for the | |
| | | Treatment of Multiple | |
| | | Myeloma Krista Kinneer, | |
| | | J . . . | |
| MOGAM Inst. patent | 2018 WO2019066435 | | Green Cross MOGAM |
| anti-BCMA | CHOI, Hye-Ji, [symbol] . . . | | Biotech |
| | ANTI-BCMA ANTIBODY | | |
| | HAVING HIGH AFFINITY | | |
| | FOR BCMA AND | | |
| | PHARMACEUTICAL | | |
| | COMPOSITION FOR | | |
| | TREATMENT OF CANCER, | | |
| | COMPRISING SAME | | |
| | 2018 US20200299395 | | |
| | CHOI, Hye-Ji; PAR . . . ANTI- | | |
| | BCMA ANTIBODY HAVING | | |
| | HIGH AFFINITY FOR | | |
| | BCMA AND | | |
| | PHARMACEUTICAL | | |
| | COMPOSITION FOR | | |
| | TREATMENT OF CANCER, | | |
| | COMPRISING SAME | | |
| | 2018 U.S. Pat. No. 11,447,559 Choi, | | |
| | Hye-Ji (Yon . . . Anti-BCMA | | |
| | antibody having high | | |
| | affinity for BCMA and | | |
| | pharmaceutical | | |
| | composition for | | |
| | treatment of cancer, | | |
| | comprising same | | |
| Nanjing Bioheng Bio | 2021 WO2022089353 LI, | | Jiangsu Pracgen Bio |
| patent anti-BCMA | Guokun, PU, R . . . BCMA- | | Nanjing Bioheng Bio |
| | TARGETING SINGLE- | | |
| | DOMAIN ANTIBODY AND | | |
| | USE THEREOF | | |
| NCI anti-BCMA CAR | | 2013 Carpenter RO, Evb . . . | NCI |
| | | B-cell maturation antigen is | |
| | | a promising target for | |
| | | adoptive T-cell therapy of | |
| | | multiple myeloma. | |
| | | ASH 2015 Remissions of | |
| | | Multiple Myeloma during a | |
| | | First-in-Humans Clinical | |
| | | Trial of T Cells Expressing | |
| | | an Anti-B-Cell Maturation | |
| | | Antigen Chimeric Antigen | |
| | | Receptor Syed Abbas Ali, | |
| | | M .. | |
| Ningbo T-Maximum | 2021 WO2022143611 | | Ningbo T-Maximum |
| patent anti-BCMA | SHANG, Xiaoyun, L . . . | | |
| | BCMA-TARGETING | | |
| | SINGLE-DOMAIN | | |
| | ANTIBODY | | |
| Nkarta patent anti-BCMA | 2022 WO2023288185 | | Nkarta |
| CAR | RAJANGAM, Kanya, . . . | | |
| | BCMA-DIRECTED | | |
| | CELLULAR | | |
| | IMMUNOTHERAPY | | |
| | COMPOSITIONS AND | | |
| | METHODS | | |
| Novartis patent anti- | 2019 WO2019241426 | | Novartis |
| BCMA CAR | ABUJOUB, Aida, FL . . . | | |
| | BCMA CHIMERIC | | |
| | ANTIGEN RECEPTORS | | |
| | AND USES THEREOF | | |
| | 2018 WO2019099639 | | |
| | GARFALL, Alfred, . . . | | |
| | BCMA-TARGETING | | |
| | CHIMERIC ANTIGEN | | |
| | RECEPTOR, CD19- | | |
| | TARGETING CHIMERIC | | |
| | ANTIGEN RECEPTOR, AND | | |
| | COMBINATION | | |
| | THERAPIES | | |
| Oricell patent anti-BCMA | 2022 WO2022228429 | | Oricell |
| CAR | CHEN, Siye BCMA- | | |
| | TARGETING CHIMERIC | | |
| | ANTIGEN RECEPTOR AND | | |
| | USE THEREOF | | |
| PersonGen anti-BCMA | 2018 NCT04650724 Early | | PersonGen |
| CAR | Phase 1 Clinical Study of T | | |
| | Cell Infusion Targeting | | |
| | BCMA Chimeric Antigen | | |
| | Receptor | | |
| Pfizer patent anti-BCMA | 2016 WO2016166630 | | Pfizer |
| CAR | KUO, Tracy Chia-C . . . | | |
| | CHIMERIC ANTIGEN | | |
| | RECEPTORS TARGETING | | |
| | B-CELL MATURATION | | |
| | ANTIGEN | | |
| Precision Biologics | 2021 WO2022035793 | | Precision Biologics |
| patent anti-BCMA | BARTSEVICH, Victo . . . | | |
| | ANTIBODIES AND | | |
| | FRAGMENTS SPECIFIC | | |
| | FOR B-CELL | | |
| | MATURATION ANTIGEN | | |
| | AND USES THEREOF | | |
| Pregene Bio anti-BCMA | 2019 WO2020038147 | 2018 NCT03661554 Early | Pregene Bio |
| CAR | ZHANG, Jishuai, L . . . ANTI- | Phase 1 BCMA Nano | |
| | BCMA SINGLE DOMAIN | Antibody CAR-T Cells for | |
| | ANTIBODIES AND | Patients With Refractory | |
| | APPLICATION THEREOF | and Relapsed Multiple | |
| | 2019 WO2020038146 | Myeloma | |
| | ZHANG, Jishuai, L . . . BCMA | | |
| | CHIMERIC ANTIGEN | | |
| | RECEPTOR BASED ON | | |
| | SINGLE DOMAIN | | |
| | ANTIBODY AND USE | | |
| | THEREOF | | |
| | 2019 US20220218746 | | |
| | ZHANG, Jishuai; L . . . BCMA | | |
| | CHIMERIC ANTIGEN | | |
| | RECEPTOR BASED ON | | |
| | SINGLE DOMAIN | | |
| | ANTIBODY AND USE | | |
| | THEREOF | | |
| | 2019 US20220251226 | | |
| | ZHANG, Jishuai; L . . . ANTI- | | |
| | BCMA SINGLE DOMAIN | | |
| | ANTIBODIES AND | | |
| | APPLICATION THEREOF | | |
| ProMab anti-BCMA | 2022 US20220356262 | 2018 Berahovich R, Zho . . . | ProMab Bio |
| | Wu, Lijun; Golubo . . . | CAR-T Cells Based on Novel | |
| | HUMANIZED BCMA | BCMA Monoclonal | |
| | ANTIBODY AND BCMA- | Antibody Block Multiple | |
| | CAR-T CELLS | Myeloma Cell Growth. | |
| | 2021 WO2022040050 | | |
| | WU, Lijun, GOLUBO . . . | | |
| | HUMANIZED BCMA | | |
| | ANTIBODY AND BCMA- | | |
| | CAR-T CELLS | | |
| | 2020 WO2021133712 | | |
| | WU, Lijun, GOLUBO . . . | | |
| | HUMANIZED BCMA | | |
| | ANTIBODY AND BCMA- | | |
| | CAR-T CELLS | | |
| | 2020 US20210015870 | | |
| | Wu, Lijun; Golubo . . . | | |
| | BCMA-CAR-T CELLS | | |
| | 2020 US20200354451 | | |
| | Wu, Lijun; Golubo . . . | | |
| | CHIMERIC ANTIGEN | | |
| | RECEPTORS COMPRISING | | |
| | A HUMAN TRANSFERRIN | | |
| | EPITOPE SEQUENCE | | |
| | 2019 WO2019195017 | | |
| | WU, Lijun, GOLUBO . . . | | |
| | BCMA-CAR-T CELLS | | |
| Protanbio patent anti- | 2021 WO2021256724 | | Korea NCC Protanbio |
| BCMA CAR | LEE, Sangjin, EOM . . . | | |
| | CHIMERIC ANTIGEN | | |
| | RECEPTOR TARGETING | | |
| | BCMA AND USE THEREOF | | |
| Salubris patent anti- | 2020 WO2021136323 LI, | | Salubris |
| BCMA | John, TANG, Y . . . | | |
| | ANTIBODIES BINDING | | |
| | BCMA AND USES | | |
| | THEREOF | | |
| SEA-BCMA | 2021 US20220025062 | 2018 NCT03582033 Phase | Seattle Genetics |
| | Sussman, Django; . . . | 1 A Safety Study of SEA- | |
| | BCMA ANTIBODIES AND | BCMA in Patients With | |
| | USE OF SAME TO TREAT | Multiple Myeloma | |
| | CANCER AND | 2017 NCT03266692 Phase | |
| | IMMUNOLOGICAL | 1 Study of ACTR087 in | |
| | DISORDERS | Combination With SEA- | |
| | 2019 US20200002431 | BCMA in Subjects With | |
| | Sussman, Django; . . . | Relapsed or Refractory | |
| | BCMA ANTIBODIES AND | Multiple Myeloma | |
| | USE OF SAME TO TREAT | AACR 2018 SEA-BCMA: A | |
| | CANCER AND | highly active enhanced | |
| | IMMUNOLOGICAL | antibody for multiple | |
| | DISORDERS | myeloma H. Van Epps, M. | |
| | 2019 U.S. Pat. No. 11,078,291 | A . . . | |
| | Sussman, Django (. . . | | |
| | BCMA antibodies and use | | |
| | of same to treat cancer | | |
| | and immunological | | |
| | disorders | | |
| | 2018 US20190023801 | | |
| | Sussman, Django; . . . | | |
| | BCMA ANTIBODIES AND | | |
| | USE OF SAME TO TREAT | | |
| | CANCER AND | | |
| | IMMUNOLOGICAL | | |
| | DISORDERS | | |
| | 2017 US20170233484 | | |
| | Sussman, Django; . . . | | |
| | BCMA ANTIBODIES AND | | |
| | USE OF SAME TO TREAT | | |
| | CANCER AND | | |
| | IMMUNOLOGICAL | | |
| | DISORDERS | | |
| | 2017 WO2017143069 | | |
| | SUSSMAN, Django, . . . | | |
| | BCMA ANTIBODIES AND | | |
| | USE OF SAME TO TREAT | | |
| | CANCER AND | | |
| | IMMUNOLOGICAL | | |
| | DISORDERS | | |
| | 2017 US20190194338 | | |
| | Sussman, Django; . . . | | |
| | BCMA ANTIBODIES AND | | |
| | USE OF SAME TO TREAT | | |
| | CANCER AND | | |
| | IMMUNOLOGICAL | | |
| | DISORDERS | | |
| Shenzhen Feipeng Bio | 2020 WO2021121250 YE, | | Shenzhen Feipeng Bio |
| patent anti-BCMA | Lijun, FENG, . . . BCMA | | |
| | BINDING ANTIBODY AND | | |
| | USE THEREOF | | |
| | 2020 US20230039487 YE, | | |
| | Lijun; FENG, . . . BCMA- | | |
| | binding antibody and use | | |
| | thereof | | |
| Shenzhen Wingor patent | 2021 WO2022033057 | | Shenzhen Wingor |
| anti-BCMA CAR | JIANG, Shu, WANG, . . . | | |
| | SINGLE-DOMAIN | | |
| | ANTIBODY-BASED BCMA | | |
| | CHIMERIC ANTIGEN | | |
| | RECEPTOR, AND | | |
| | APPLICATION THEREOF | | |
| Single Cell Tech. patent | 2019 WO2020073917 | | Single Cell Tech. |
| anti-BCMA | TANG, Leyan, SCHU . . . | | |
| | ANTI-BCMA ANTIBODIES | | |
| | 2019 US20210332145 | | |
| | TANG, Leyan; SCHU . . . | | |
| | ANTI-BCMA ANTIBODIES | | |
| Sloan-Kettering patent | 2021 US20220315660 | | Eureka Sloan-Kettering |
| anti-BCMA | Brentjens, Renier . . . | | |
| | ANTIBODIES TARGETING | | |
| | B-CELL MATURATION | | |
| | ANTIGEN AND METHODS | | |
| | OF USE | | |
| | 2019 US20200123266 | | |
| | Brentjens, Renier . . . | | |
| | ANTIBODIES TARGETING | | |
| | B-CELL MATURATION | | |
| | ANTIGEN AND METHODS | | |
| | OF USE | | |
| | 2019 U.S. Pat. No. 10,947,314 | | |
| | Brentjens, Renier . . . | | |
| | Antibodies targeting b- | | |
| | cell maturation antigen | | |
| | and methods of use | | |
| | 2017 US20180118842 | | |
| | Brentjens, Renier . . . | | |
| | ANTIBODIES TARGETING | | |
| | B-CELL MATURATION | | |
| | ANTIGEN AND METHODS | | |
| | OF USE | | |
| | 2017 U.S. Pat. No. 10,562,972 | | |
| | Brentjens, Renier . . . | | |
| | Antibodies targeting B- | | |
| | cell maturation antigen | | |
| | and methods of use | | |
| | 2015 WO2016090327 | | |
| | BRENTJENS, Renier . . . | | |
| | ANTIBODIES TARGETING | | |
| | B-CELL MATURATION | | |
| | ANTIGEN AND METHODS | | |
| | OF USE | | |
| Sorrento patent anti- | 2022 US20220251168 Ji, | | Sorrento |
| BCMA | Henry Hongjun . . . Dimeric | | |
| | Antigen Receptors (DAR) | | |
| | that Bind BCMA | | |
| | 2022 WO2022184082 | | |
| | ZHU, Tong, KHASAN . . . | | |
| | ANTIBODY-DRUG | | |
| | CONJUGATES | | |
| | COMPRISING AN ANTI- | | |
| | BCMA ANTIBODY | | |
| | 2021 US20210388097 | | |
| | Zhou, Heyue; Cao, . . . | | |
| | Antigen Binding Proteins | | |
| | that Bind BCMA | | |
| | 2020 WO2021046445 JI, | | |
| | Henry Hongjun . . . | | |
| | DIMERIC ANTIGEN | | |
| | RECEPTORS (DAR) THAT | | |
| | BIND BCMA | | |
| | 2020 WO2020176549 | | |
| | ZHOU, Heyue, CAO, . . . | | |
| | ANTIGEN BINDING | | |
| | PROTEINS THAT BIND | | |
| | BCMA | | |
| Sutro patent anti-BCMA | 2020 WO2020227110 | | Sutro |
| | LEE, John, STAFFO . . . | | |
| | ANTI-BCMA ANTIBODY | | |
| | CONJUGATE, | | |
| | COMPOSITIONS | | |
| | COMPRISING THE SAME, | | |
| | AND METHODS OF | | |
| | MAKING AND USING THE | | |
| | SAME | | |
| | 2020 WO2020227105 | | |
| | LEE, John, STAFFO . . . | | |
| | ANTI-BCMA ANTIBODY | | |
| | CONJUGATES | | |
| | 2020 US20220362394 | | |
| | LEE, John; STAFFO . . . | | |
| | ANTI-BCMA ANTIBODY | | |
| | CONJUGATES | | |
| | 2019 WO2019190969 | | |
| | YAM, Alice, STAFF . . . ANTI- | | |
| | BCMA RECEPTOR | | |
| | ANTIBODIES, | | |
| | COMPOSITIONS | | |
| | COMPRISING ANTI BCMA | | |
| | RECEPTOR ANTIBODIES | | |
| | AND METHODS OF | | |
| | MAKING AND USING | | |
| | ANTI-BCMA ANTIBODIES | | |
| | 2019 US20210130483 | | |
| | YAM, Alice; STAFF . . . ANTI- | | |
| | BCMA RECEPTOR | | |
| | ANTIBODIES, | | |
| | COMPOSITIONS | | |
| | COMPRISING ANTI BCMA | | |
| | RECEPTOR ANTIBODIES | | |
| | AND METHODS OF | | |
| | MAKING AND USING | | |
| | ANTI-BCMA ANTIBODIES | | |
| Suzhou Qin patent anti- | 2020 WO2021068761 LI, | | Suzhou Qin |
| BCMA | Ning, CAO, Gu . . . | | |
| | HUMANIZED | | |
| | MONOCLONAL | | |
| | ANTIBODY TARGETING | | |
| | BCMA AND HAVING | | |
| | HUMAN MONKEY CROSS- | | |
| | REACTIVITY | | |
| TBL-CLN1 | 2011 US20120027763 | | Transgene Biotek |
| | KOLLIPARA, Kotesw . . . | | |
| | Antibody for Targeted | | |
| | induction of Apoptosis, | | |
| | CDC and ADCC mediated | | |
| | killing of Cancer cells, | | |
| | TBL-CLN1 | | |
| | 2011 WO2011108008 | | |
| | KOLLIPARA, Kotesw . . . | | |
| | ANTIBODY FOR | | |
| | TARGETED INDUCTION | | |
| | OF APOPTOSIS, CDC AND | | |
| | ADCC MEDIATED KILLING | | |
| | OF CANCER CELLS, TBL- | | |
| | CLN1 | | |
| TSD Life Sci. patent anti- | 2019 WO2020004937 | | TSD Life Sci. |
| BCMA | KIM, Juhee, CHANG . . . | | |
| | ANTI-BCMA ANTIBODY - | | |
| | DRUG CONJUGATE AND | | |
| | USE THEREOF | | |
| U. Penn. anti-BCMA CAR | | 2015 NCT02546167 Phase | U. Penn. |
| | | 0 CART-BCMA Cells for | |
| | | Multiple Myeloma | |
| UCL patent anti-BCMA | 2016 WO2016151315 | | UCL |
| CAR | PULÉ, Martin, COR . . . | | |
| | CHIMERIC ANTIGEN | | |
| | RECEPTOR | | |
| | 2014 WO2015052538 | | |
| | PULÉ, Martin, YON . . . | | |
| | CHIMERIC ANTIGEN | | |
| | RECEPTOR | | |
| Virocan patent anti- | 2017 WO2017130223 | | Virocan |
| BCMA CAR | BATCHU, Ramesh B. . . . A | | |
| | CHIMERIC ANTIGEN | | |
| | RECEPTOR SPECIFIC TO B- | | |
| | CELL MATURATION | | |
| | ANTIGEN, A | | |
| | RECOMBINANT | | |
| | EXPRESSION VECTOR | | |
| | AND A METHOD | | |
| | THEREOF | | |
| | 2017 US20200405758 | | |
| | BATCHU, Ramesh B. . . . A | | |
| | CHIMERIC ANTIGEN | | |
| | RECEPTOR SPECIFIC TO B- | | |
| | CELL MATURATION | | |
| | ANTIGEN, A | | |
| | RECOMBINANT | | |
| | EXPRESSION VECTOR | | |
| | AND A METHOD | | |
| | THEREOF | | |
| Wuhan YZY Biopharma | 2020 WO2022126416 | | Wuhan YZY Biopharma |
| anti-BCMA | FANG, Lijuan, SHI . . . ANTI- | | |
| | BCMA ANTIBODY, | | |
| | PREPARATION METHOD | | |
| | THEREFOR AND | | |
| | APPLICATION THEREOF | | |
| Xi'an Yufan Bio patent | 2017 WO2019085102 | | Xi'an Yufan Bio |
| anti-BCMA CAR | LONG, Fei, CHEN, . . . | | |
| | BCMA-SPECIFIC | | |
| | CHIMERIC ANTIGEN | | |
| | RECEPTOR T CELL AND | | |
| | APPLICATION THEREOF | | |
| ABL Bio patent anti-4- | 2020 WO2021118246 | | ABL Bio |
| 1BB / BCMA | PARK, Kyung Jin, . . . ANTI- | | |
| | BCMA/ANTI-4-1BB | | |
| | BISPECIFIC ANTIBODIES | | |
| | AND USES THEREOF | | |
| | 2019 WO2021132746 | | |
| | PARK, Kyungjin, K . . . ANTI- | | |
| | BCMA/ANTI-4-1BB | | |
| | BISPECIFIC ANTIBODIES | | |
| | AND USES THEREOF | | |
| alnuctamab | | | |
| AMG 701 | | | Amgen |
| Beijing Wisdomab Bio. | 2019 WO2020252907 | | Beijing Wisdomab Bio. |
| patent anti-CD3E / BCMA | LIU, Zhigang, WAN . . . | | |
| | ANTI-CD3E/BCMA | | |
| | BISPECIFIC ANTIBODY | | |
| | AND USE THEREOF | | |
| CC-93269 | 2021 US20220017631 Vu, | 2018 NCT03486067 Phase | Celgene Engmab |
| | Minh Diem; St . . . | 1 Study of CC-93269, a | |
| | BISPECIFIC ANTIBODY | BCMA × CD3 T Cell | |
| | AGAINST BCMA AND CD3 | Engaging Antibody, in | |
| | AND AN | Subjects With Relapsed | |
| | IMMUNOLOGICAL DRUG | and Refractory Multiple | |
| | FOR COMBINED USE IN | Myeloma | |
| | TREATING MULTIPLE | ASH 2019 Targeting B-Cell | |
| | MYELOMA | Maturation Antigen | |
| | 2021 WO2021163329 | (BCMA) with CC-93269, a | |
| | MENSAH, Kofi, PLE . . . | 2 + 1 T Cell Engager, Elicits | |
| | ANTI-BCMA THERAPY IN | Significant Apoptosis in | |
| | AUTOIMMUNE | Diffuse Large B-Cell | |
| | DISORDERS | Lymphoma Preclinical | |
| | 2020 US20200385471 Vu, | Models Patrick R. Hagner . . . | |
| | Minh Diem; St . . . | | |
| | BISPECIFIC ANTIBODIES | | |
| | AGAINST CD3EPSILON | | |
| | AND BCMA FOR USE IN | | |
| | TREATMENT OF DISEASES | | |
| | 2020 WO2020219978 VU, | | |
| | Minh Diem, CH . . . | | |
| | BCMA/CD3 BISPECIFIC | | |
| | TRIVALENT T-CELL | | |
| | ENGAGING (TCE) | | |
| | ANTIBODIES AND THEIR | | |
| | USE TO TREAT | | |
| | HEMATOLOGICAL | | |
| | MALIGNANCIES | | |
| | 2019 WO2020089437 | | |
| | PAIVA, Bruno Davi . . . | | |
| | COMBINATION THERAPY | | |
| | 2019 WO2019226761 | | |
| | PIERCE, Daniel, W . . . | | |
| | ANTIPROLIFERATIVE | | |
| | COMPOUNDS AND | | |
| | BISPECIFIC ANTIBODY | | |
| | AGAINST BCMA AND CD3 | | |
| | FOR COMBINED USE | | |
| | 2019 US20190381035 | | |
| | Pierce, Daniel W. . . . | | |
| | ANTIPROLIFERATIVE | | |
| | COMPOUNDS AND | | |
| | BISPECIFIC ANTIBODY | | |
| | AGAINST BCMA AND CD3 | | |
| | FOR COMBINED USE | | |
| | 2019 U.S. Pat. No. 11,439,637 Pierce, | | |
| | Daniel W. . . . | | |
| | Antiproliferative | | |
| | compounds and bispecific | | |
| | antibody against BCMA | | |
| | and CD3 for combined | | |
| | use | | |
| | 2017 WO2018083204 VU, | | |
| | Minh Diem, ST . . . | | |
| | BISPECIFIC ANTIBODY | | |
| | AGAINST BCMA AND CD3 | | |
| | AND AN | | |
| | IMMUNOLOGICAL DRUG | | |
| | FOR COMBINED USE IN | | |
| | TREATING MULTIPLE | | |
| | MYELOMA | | |
| | 2017 US20190263920 Vu, | | |
| | Minh Diem; St . . . | | |
| | BISPECIFIC ANTIBODY | | |
| | AGAINST BCMA AND CD3 | | |
| | AND AN | | |
| | IMMUNOLOGICAL DRUG | | |
| | FOR COMBINED USE IN | | |
| | TREATING MULTIPLE | | |
| | MYELOMA | | |
| | 2017 U.S. Pat. No. 11,124,577 Vu, | | |
| | Minh Diem (Wo . . . | | |
| | Bispecific antibody | | |
| | against BCMA and CD3 | | |
| | and an immunological | | |
| | drug for combined use in | | |
| | treating multiple | | |
| | myeloma | | |
| Chugai patent anti-IL-6 / | 2019 US20200048627 | | Chugai |
| BCMA | IGAWA, Tomoyuki; . . . | | |
| | ANTIGEN-BINDING | | |
| | MOLECULE CAPABLE OF | | |
| | BINDING TO TWO OR | | |
| | MORE ANTIGEN | | |
| | MOLECULES REPEATEDLY | | |
| CM336 | 2021 WO2022117045 XU, | | Keymed |
| | Gang, CHEN, B . . . | | |
| | DEVELOPMENT AND | | |
| | APPLICATION OF T-CELL | | |
| | ENGAGER THERAPEUTIC | | |
| | AGENT | | |
| Cytoimmune patent anti- | 2022 US20220281982 | | Cytoimmune |
| NKG2D / BCMA | Caligiuri, Michae . . . | | |
| | BISPECIFIC ANTIBODY | | |
| | CAR CELL | | |
| | IMMUNOTHERAPY | | |
| | 2021 WO2022046788 | | |
| | ZHANG, Lei BISPECIFIC | | |
| | ANTIBODY CAR CELL | | |
| | IMMUNOTHERAPY | | |
| | 2020 WO2021050591 | | |
| | CALIGIURI, Michae . . . | | |
| | BISPECIFIC ANTIBODY | | |
| | CAR CELL | | |
| | IMMUNOTHERAPY | | |
| | 2019 WO2019178576 YU, | | |
| | Jianhua, CALI . . . | | |
| | BISPECIFIC ANTIBODY | | |
| | CAR CELL | | |
| | IMMUNOTHERAPY | | |
| | 2019 US20210087275 YU, | | |
| | Jianhua; CALI . . . | | |
| | BISPECIFIC ANTIBODY | | |
| | CAR CELL | | |
| | IMMUNOTHERAPY | | |
| elranatamab | 2021 WO2022053990 | 2022 NCT05675449 Phase | Pfizer |
| | BARDY BOUXIN, Nat . . . | 1 A Clinical Trial of Three | |
| | METHODS, THERAPIES | Medicines (Elranatamab | |
| | AND USES FOR TREATING | Plus Carfilzomib and | |
| | CANCER | Dexamethasone) in People | |
| | 2021 WO2021229507 | With Relapsed Refractory | |
| | BARDY BOUXIN, Nat . . . | Multiple Myeloma | |
| | METHODS, THERAPIES | 2022 NCT05565391 A | |
| | AND USES FOR TREATING | Study to Learn About the | |
| | CANCER | Medicine (Called | |
| | 2020 US20210188991 | Elranatamab) in People | |
| | KUO, Tracy Chia-C . . . | With Relapsed Refractory | |
| | THERAPEUTIC | Multiple Myeloma | |
| | ANTIBODIES AND THEIR | 2022 NCT05462639 | |
| | USES | Elranatamab Expanded | |
| | 2020 U.S. Pat. No. 11,155,630 Kuo, | Access Protocol in Adults | |
| | Tracy Chia-C . . . | With Relapsed/Refractory | |
| | Therapeutic antibodies | Multiple Myeloma | |
| | and their uses | 2022 NCT05317416 Phase | |
| | 2020 US20210054087 | 3 Study With Elranatamab | |
| | KUO, Tracy Chia-C . . . | Versus Lenalidomide in | |
| | THERAPEUTIC | Patients With Newly | |
| | ANTIBODIES AND THEIR | Diagnosed Multiple | |
| | USES | Myeloma After Transplant | |
| | 2019 US20210017254 | 2021 NCT05228470 Phase | |
| | ISKRA, Timothy; S . . . | 2 MagnetisMM-8: Study Of | |
| | Antibody Purification | Elranatamab (PF- | |
| | 2018 US20180298108 | 06863135) Monotherapy in | |
| | KUO, Tracy Chia-C . . . | Chinese Participants With | |
| | THERAPEUTIC | Refractory Multiple | |
| | ANTIBODIES AND THEIR | Myeloma | |
| | USES | 2021 NCT05014412 Phase | |
| | 2018 U.S. Pat. No. 10,793,635 Kuo, | 2 MagnetisMM-9: Study of | |
| | Tracy Chia-C . . . | Elranatamab (PF- | |
| | Therapeutic antibodies | 06863135) Monotherapy in | |
| | and their uses | Participants With | |
| | 2018 US20180171018 | Relapsed/Refractory | |
| | KUO, Tracy Chia-C . . . | Multiple Myeloma | |
| | THERAPEUTIC | 2021 NCT05020236 Phase | |
| | ANTIBODIES AND THEIR | 3 Study of Elranatamab | |
| | USES | (PF-06863135) | |
| | 2018 U.S. Pat. No. 10,040,860 Kuo, | Monotherapy and | |
| | Tracy Chia-C . . . | Elranatamab + | |
| | Therapeutic antibodies | Daratumumab Versus | |
| | and their uses | Daratumumab + | |
| | 2016 US20160297885 | Pomalidomide + | |
| | KUO, Tracy Chia-C . . . | Dexamethasone in | |
| | THERAPEUTIC | Participants With | |
| | ANTIBODIES AND THEIR | Relapsed/Refractory | |
| | USES | Multiple Myeloma | |
| | 2016 WO2016166629 | 2021 NCT04798586 Phase | |
| | KUO, Tracy Chia-C . . . | 1 Study of PF 06863135 in | |
| | THERAPEUTIC | Japanese Participants With | |
| | ANTIBODIES AND THEIR | Multiple Myeloma | |
| | USES | 2021 NCT04649359 Phase | |
| | 2016 U.S. Pat. No. 9,969,809 Kuo, | 2 Study of PF-06863135 | |
| | Tracy Chia-C . . . | Monotherapy in | |
| | Therapeutic antibodies | Participants With Multiple | |
| | and their uses | Myeloma Who Are | |
| | | Refractory to at Least One | |
| | | PI, One IMiD and One Anti- | |
| | | CD38 mAb | |
| | | 2017 NCT03269136 Phase | |
| | | 1 Phase 1 Study Of PF- | |
| | | 06863135, A BCMA- CD3 | |
| | | Bispecific Ab, As A Single | |
| | | Agent And In Combination | |
| | | With Either PF-06801591 | |
| | | Or Lenalidomide In | |
| | | Relapse/ Refractory | |
| | | Multiple Myeloma | |
| | | 2019 Panowski SH, Kuo . . . | |
| | | Preclinical Efficacy and | |
| | | Safety Comparison of CD3 | |
| | | Bispecific and ADC | |
| | | Modalities Targeting BCMA | |
| | | for the Treatment of | |
| | | Multiple Myeloma. | |
| | | 2019 Ying Wang Y, Luon . . . | |
| | | Addressing soluble target | |
| | | interference in the | |
| | | development of a | |
| | | functional assay for the | |
| | | detection of neutralizing | |
| | | antibodies against a BCMA- | |
| | | CD3 bispecific antibody. | |
| EM801 | 2021 US20220002427 Vu, | ASH 2015 Target | Engmab Roche |
| | Minh Diem; St . . . | Expression, Preclinical | |
| | BISPECIFIC ANTIBODY | Activity and Mechanism of | |
| | AGAINST BCMA AND CD3 | Action of EM801: A Novel | |
| | AND AN | First-in-Class Bcma T-Cell | |
| | IMMUNOLOGICAL DRUG | Bispecific Antibody for the | |
| | FOR COMBINED USE IN | Treatment of Multiple | |
| | TREATING MULTIPLE | Myeloma Anja Seckinger, | |
| | MYELOMA | M . . . | |
| | 2021 US20220017631 Vu, | ASH 2015 A New Class of T- | |
| | Minh Diem; St . . . | Cell Bispecific Antibodies | |
| | BISPECIFIC ANTIBODY | for the Treatment of | |
| | AGAINST BCMA AND CD3 | Multiple Myeloma, Binding | |
| | AND AN | to B Cell Maturation | |
| | IMMUNOLOGICAL DRUG | Antigen and CD3 and | |
| | FOR COMBINED USE IN | Showing Potent, Specific | |
| | TREATING MULTIPLE | Antitumor Activity in | |
| | MYELOMA | Myeloma Cells and Long | |
| | 2020 WO2021092056 | Duration of Action in | |
| | BURGESS, Michael, . . . | Cynomolgus Monkeys | |
| | METHODS OF | | |
| | TREATMENT WITH | | |
| | ANTIBODIES AGAINST | | |
| | BCMA AND CD3 | | |
| | 2020 WO2021092060 | | |
| | HAGNER, Patrick, . . . | | |
| | METHODS OF | | |
| | TREATMENT | | |
| | 2020 US20200385471 Vu, | | |
| | Minh Diem; St . . . | | |
| | BISPECIFIC ANTIBODIES | | |
| | AGAINST CD3EPSILON | | |
| | AND BCMA FOR USE IN | | |
| | TREATMENT OF DISEASES | | |
| | 2017 WO2018083204 VU, | | |
| | Minh Diem, ST . . . | | |
| | BISPECIFIC ANTIBODY | | |
| | AGAINST BCMA AND CD3 | | |
| | AND AN | | |
| | IMMUNOLOGICAL DRUG | | |
| | FOR COMBINED USE IN | | |
| | TREATING MULTIPLE | | |
| | MYELOMA | | |
| | 2017 US20190263920 Vu, | | |
| | Minh Diem; St . . . | | |
| | BISPECIFIC ANTIBODY | | |
| | AGAINST BCMA AND CD3 | | |
| | AND AN | | |
| | IMMUNOLOGICAL DRUG | | |
| | FOR COMBINED USE IN | | |
| | TREATING MULTIPLE | | |
| | MYELOMA | | |
| | 2017 U.S. Pat. No. 11,124,577 Vu, | | |
| | Minh Diem (Wo . . . | | |
| | Bispecific antibody | | |
| | against BCMA and CD3 | | |
| | and an immunological | | |
| | drug for combined use in | | |
| | treating multiple | | |
| | myeloma | | |
| | 2017 WO2018060301 | | |
| | KLEIN, Christian, . . . | | |
| | BISPECIFIC ANTIBODIES | | |
| | AGAINST CD3 | | |
| | 2015 WO2016087531 VU, | | |
| | Minh, Diem, . . . | | |
| | BISPECIFIC ANTIBODIES | | |
| | AGAINST CD3EPSILON | | |
| | AND BCMA FOR USE IN | | |
| | TREATMENT OF DISEASES | | |
| | 2015 US20170327580 Vu, | | |
| | Minh Diem; St . . . | | |
| | BISPECIFIC ANTIBODIES | | |
| | AGAINST CD3 EPSILON | | |
| | AND BCMA FOR USE IN | | |
| | TREATMENT OF DISEASES | | |
| | 2015 WO2016079177 VU, | | |
| | Minh, Diem, . . . | | |
| | BISPECIFIC ANTIBODIES | | |
| | AGAINST CD3EPSILON | | |
| | AND BCMA | | |
| | 2015 US20170327579 | | |
| | VU, MINH DIEM; ST . . . | | |
| | BISPECIFIC ANTIBODIES | | |
| | AGAINST CD3EPSILON | | |
| | AND BCMA | | |
| | 2015 WO2016020332 VU, | | |
| | Minh, Diem, . . . | | |
| | BISPECIFIC ANTIBODIES | | |
| | AGAINST CD3EPSILON | | |
| | AND BCMA | | |
| | 2015 US20170306036 | | |
| | VU, Minh Diem; ST . . . | | |
| | BISPECIFIC ANTIBODIES | | |
| | AGAINST CD3EPSILON | | |
| | AND BCMA | | |
| | 2015 U.S. Pat. No. 10,253,104 Vu, | | |
| | Minh Diem (Wo . . . | | |
| | Bispecific antibodies | | |
| | against CD3ϵ and BCMA | | |
| | 2014 WO2014122144 VU, | | |
| | Minh Diem, ST . . . | | |
| | BISPECIFIC ANTIBODIES | | |
| | AGAINST CD3ε AND | | |
| | BCMA | | |
| | 2014 US20150376287 | | |
| | VU, Minh Diem; ST . . . | | |
| | BISPECIFIC ANTIBODIES | | |
| | AGAINST CD3 AND BCMA | | |
| | 2014 U.S. Pat. No. 10,077,315 Vu, | | |
| | Minh Diem (Wo . . . | | |
| | Bispecific antibodies | | |
| | against CD3 and BCMA | | |
| EMB-06 | 2020 WO2021104371 | 2021 NCT04735575 Phase | EpimAb |
| | WU, Chengbin, WU, . . . | 1/Phase 2 A Ph1/2 Study of | |
| | ANTIBODIES TO CD3 AND | EMB-06 in Participants | |
| | BCMA, AND BISPECIFIC | With Recurrent or | |
| | BINDING PROTEINS | Refractory Myeloma | |
| | MADE THEREFROM | | |
| | 2020 US20230002489 | | |
| | WU, Chengbin; WU, . . . | | |
| | ANTIBODIES TO CD3 AND | | |
| | BCMA, AND BISPECIFIC | | |
| | BINDING PROTEINS | | |
| | MADE THEREFROM | | |
| Guangzhou Excelmab | 2022 WO2023001154 | | Guangzhou Excelmab |
| patent anti-CD3 / BCMA | ZHANG, Wenjun B7-H3 | | |
| | ANTIBODY AND USE | | |
| | THEREOF | | |
| Hansoh patent anti- | 2021 WO2021209066 | | Hansoh Pharma |
| BCMA / CD3 | HUA, Haiqing, BAO . . . | | |
| | SPECIFIC ANTIGEN | | |
| | BINDING MOLECULE, | | |
| | AND PREPARATION | | |
| | METHOD AND | | |
| | PHARMACEUTICAL USE | | |
| | THEREFOR | | |
| HBM7020 | 2020 US20220340673 | | Harbour Biomed Ltd. |
| | SHI, Lei; ZHANG, . . . | | |
| | ANTIBODY TARGETING | | |
| | BCMA, BISPECIFIC | | |
| | ANTIBODY, AND USE | | |
| | THEREOF | | |
| Innovent patent anti- | 2021 WO2022135468 PU, | | Innovent |
| BCMA / CD3 | Pu, CHEN, Bin . . . ANTI- | | |
| | BCMA × CD3 BISPECIFIC | | |
| | ANTIBODY AND USE | | |
| | THEREOF | | |
| Kite patent anti-TACI / | 2022 WO2022221028 | | Kite |
| BCMA | BELK, Jonathan | | |
| | TACI/BCMA DUAL | | |
| | BINDING MOLECULES | | |
| | 2022 US20220354892 | | |
| | Belk, Jonathan; C . . . TACI | | |
| | BINDING MOLECULES | | |
| Novartis patent anti- | 2021 WO2022097090 | | Novartis |
| BCMA / CD3 | AARDALEN, Kimberl . . . | | |
| | DOSING REGIMEN FOR | | |
| | COMBINATION | | |
| | THERAPIES WITH | | |
| | MULTISPECIFIC | | |
| | ANTIBODIES TARGETING | | |
| | B-CELL MATURATION | | |
| | ANTIGEN AND GAMMA | | |
| | SECRETASE INHIBITORS | | |
| | 2020 WO2020261093 | | |
| | AARDALEN, Kimberl . . . | | |
| | DOSING REGIMEN AND | | |
| | COMBINATION | | |
| | THERAPIES FOR | | |
| | MULTISPECIFIC | | |
| | ANTIBODIES TARGETING | | |
| | B-CELL MATURATION | | |
| | ANTIGEN | | |
| | 2020 US20220332821 | | |
| | AARDALEN, Kimberl . . . | | |
| | DOSING REGIMEN AND | | |
| | COMBINATION | | |
| | THERAPIES FOR | | |
| | MULTISPECIFIC | | |
| | ANTIBODIES TARGETING | | |
| | B-CELL MATURATION | | |
| | ANTIGEN | | |
| | 2020 WO2020236795 | | |
| | GRANDA, Brian, BL . . . | | |
| | TRISPECIFIC BINDING | | |
| | MOLECULES AGAINST | | |
| | BCMA AND USES | | |
| | THEREOF | | |
| | 2019 WO2019229701 | | |
| | ABUJOUB, Aida, BL . . . | | |
| | BINDING MOLECULES | | |
| | AGAINST BCMA AND | | |
| | USES THEREOF | | |
| | 2018 WO2018201051 | | |
| | DALEY, Michael, L . . . | | |
| | BCMA-TARGETING | | |
| | AGENT, AND | | |
| | COMBINATION THERAPY | | |
| | WITH A GAMMA | | |
| | SECRETASE INHIBITOR | | |
| | 2018 US20200179511 | | |
| | Daley, Michael; L . . . | | |
| | BCMA-TARGETING | | |
| | AGENT, AND | | |
| | COMBINATION THERAPY | | |
| | WITH A GAMMA | | |
| | SECRETASE INHIBITOR | | |
| pacanalotamab | | | Amgen |
| pavurutamab | 2021 WO2022103781 | 2017 NCT03287908 Phase | Amgen Boehringer |
| | SHARMA, Anjali, M . . . | 1 A Phase 1 Study of AMG | Micromet |
| | METHODS FOR | 701 in Subjects With | |
| | ADMINISTERING A BCMA × | Multiple Myeloma | |
| | CD3 BINDING | 2015 NCT02514239 Phase | |
| | MOLECULE | 1 Phase I Dose Escalation | |
| | 2020 WO2021094000 | of i.v. and s.c. BI 836909 | |
| | STIEGLMAIER, Juli . . . | Monotherapy in Last Line | |
| | DOSING REGIMEN FOR | Multiple Myeloma Patients | |
| | ANTI-BCMA AGENTS | 2020 Topp MS, Duell J, . . . | |
| | 2019 WO2020025596 | Anti-B-Cell Maturation | |
| | ZUGMAIER, Gerhard . . . | Antigen BiTE Molecule | |
| | DOSING REGIMEN FOR | AMG 420 Induces | |
| | BCMA-CD3 BISPECIFIC | Responses in Multiple | |
| | ANTIBODIES | Myeloma. | |
| | 2019 US20210284732 | 2017 Hipp S, Tai YT, B . . . A | |
| | Zugmaier, Gerhard . . . | novel BCMA/CD3 bispecific | |
| | DOSING REGIMEN FOR | T-cell engager for the | |
| | BCMA-CD3 BISPECIFIC | treatment of multiple | |
| | ANTIBODIES | myeloma induces selective | |
| | 2019 US20200048357 | lysis in vitro and in vivo. | |
| | Raum, Tobias; Mun . . . | 2016 Hipp S, Tai YT, B . . . A | |
| | BCMA AND CD3 | novel BCMA/CD3 bispecific | |
| | BISPECIFIC T CELL | T-cell engager for the | |
| | ENGAGING ANTIBODY | treatment of multiple | |
| | CONSTRUCTS | myeloma induces selective | |
| | 2019 U.S. Pat. No. 11,352,433 Raum, | lysis in vitro and in vivo. | |
| | Tobias (Mun . . . BCMA and | ASCO 2016 Phase 1 dose- | |
| | CD3 bispecific T cell | escalation study of BI | |
| | engaging antibody | 836909, an anti-BCMA bi- | |
| | constructs | specific T-cell engager, in | |
| | 2017 US20170218077 | relapsed and/or refractory | |
| | Raum, Tobias; Mun . . . | multiple myeloma (RRMM) | |
| | BCMA AND CD3 | Max S. Topp, Mich . . . | |
| | BISPECIFIC T CELL | ASH 2015 BI 836909, a | |
| | ENGAGING ANTIBODY | Novel Bispecific T Cell | |
| | CONSTRUCTS | Engager for the Treatment | |
| | 2017 WO2017134134 | of Multiple Myeloma | |
| | RAUM, Tobias, MUE . . . | Induces Highly Specific and | |
| | BCMA and CD3 Bispecific | Efficacious Lysis of Multiple | |
| | T Cell Engaging Antibody | Myeloma Cells in Vitro and | |
| | Constructs | Shows Anti-Tumor Activity | |
| | 2017 U.S. Pat. No. 10,301,391 Raum, | in Vivo Susanne Hipp, | |
| | Tobias (Mun . . . BCMA and | PhD . . . | |
| | CD3 bispecific T cell | | |
| | engaging antibody | | |
| | constructs | | |
| | 2014 WO2014140248 | | |
| | KUFER, Peter, RAU . . . | | |
| | BINDING MOLECULES | | |
| | FOR BCMA AND CD3 | | |
| | 2012 WO2013072415 | | |
| | KUFER, Peter, RAU . . . | | |
| | BINDING MOLECULES | | |
| | FOR BCMA AND CD3 | | |
| | 2012 WO2013072406 | | |
| | KUFER, Peter, RAU . . . | | |
| | BINDING MOLECULES | | |
| | FOR BCMA AND CD3 | | |
| REGN5458 | 2022 US20220306758 | 2022 NCT05106387 An | Regeneron |
| | Smith, Eric; Olso . . . | Observational Extension | |
| | Bispecific Anti-BCMA × | Study for Adult Patients | |
| | Anti-CD3 Antibodies and | Treated in Study R5459-RT- | |
| | Uses Thereof | 1944 Who Receive A | |
| | 2020 WO2021113701 | Kidney Transplant | |
| | LOWY, Israel, STE . . . | 2022 NCT05137054 Phase | |
| | METHODS OF TREATING | 1 REGN5458 (Anti-BCMA × | |
| | MULTIPLE MYELOMA | Anti-CD3 Bispecific | |
| | WITH BISPECIFIC ANTI- | Antibody) Plus Other | |
| | BCMA × ANTI-CD3 | Cancer Treatments for | |
| | ANTIBODIES | Participants With | |
| | 2020 US20210206865 | Relapsed/Refractory | |
| | Lowy, Israel; Ste . . . | Multiple Myeloma | |
| | Methods of Treating | 2018 NCT03761108 Phase | |
| | Multiple Myeloma with | 1/Phase 2 First in Human | |
| | Bispecific Anti-BCMA × | (FIH) Study of REGN5458 in | |
| | Anti-CD3 Antibodies | Patients With Relapsed or | |
| | 2020 WO2020191346 | Refractory Multiple | |
| | ASRAT, Seblewonge . . . | Myeloma | |
| | COMBINATION OF IL-4/IL- | 2022 Zonder JA, Richte . . . | |
| | 13 PATHWAY INHIBITORS | MM-087 Early, Deep, and | |
| | AND PLASMA CELL | Durable Responses, and | |
| | ABLATION FOR TREATING | Low Rates of Cytokine | |
| | ALLERGY | Release Syndrome With | |
| | 2020 US20200345843 | REGN5458, a BCMA × CD3 | |
| | ASRAT, Seblewonge . . . | Bispecific Antibody, in a | |
| | COMBINATION OF IL-4/IL- | Phase 1/2 First-In-Human | |
| | 13 PATHWAY INHIBITORS | Study in Patients With | |
| | AND PLASMA CELL | Relapsed/Refractory | |
| | ABLATION FOR TREATING | Multiple Myeloma. | |
| | ALLERGY | 2021 DiLillo DJ, Olson . . . A | |
| | 2019 US20200024356 | BCMA × CD3 bispecific T cell- | |
| | Smith, Eric; Olso . . . | engaging antibody | |
| | Bispecific Anti-BCMA × | demonstrates robust | |
| | Anti-CD3 Antibodies and | antitumor efficacy similar | |
| | Uses Thereof | to that of anti-BCMA CAR T | |
| | 2019 WO2020018820 | cells. | |
| | SMITH, Eric, OLSO . . . | | |
| | BISPECIFIC ANTI-BCMA × | | |
| | ANTI-CD3 ANTIBODIES | | |
| | AND USES THEREOF | | |
| | 2019 WO2020018825 | | |
| | DILILLO, David, D . . . | | |
| | CHIMERIC ANTIGEN | | |
| | RECEPTORS WITH BCMA | | |
| | SPECIFICITY AND USES | | |
| | THEREOF | | |
| | 2019 U.S. Pat. No. 11,384,153 Smith, | | |
| | Eric (New . . . Bispecific | | |
| | anti-BCMA × anti-CD3 | | |
| | antibodies and uses | | |
| | thereof | | |
| REGN5459 | 2022 US20220306758 | 2022 NCT05106387 An | Regeneron |
| | Smith, Eric; Olso . . . | Observational Extension | |
| | Bispecific Anti-BCMA × | Study for Adult Patients | |
| | Anti-CD3 Antibodies and | Treated in Study R5459-RT- | |
| | Uses Thereof | 1944 Who Receive A | |
| | 2020 WO2021113701 | Kidney Transplant | |
| | LOWY, Israel, STE . . . | 2019 NCT04083534 Phase | |
| | METHODS OF TREATING | 1 First In Human (FIH) | |
| | MULTIPLE MYELOMA | Study of REGN5459 in | |
| | WITH BISPECIFIC ANTI- | Patients With Relapsed or | |
| | BCMA × ANTI-CD3 | Refractory Multiple | |
| | ANTIBODIES | Myeloma (MM) | |
| | 2020 US20210206865 | | |
| | Lowy, Israel; Ste . . . | | |
| | Methods of Treating | | |
| | Multiple Myeloma with | | |
| | Bispecific Anti-BCMA × | | |
| | Anti-CD3 Antibodies | | |
| | 2020 WO2020191346 | | |
| | ASRAT, Seblewonge . . . | | |
| | COMBINATION OF IL-4/IL- | | |
| | 13 PATHWAY INHIBITORS | | |
| | AND PLASMA CELL | | |
| | ABLATION FOR TREATING | | |
| | ALLERGY | | |
| | 2020 US20200345843 | | |
| | ASRAT, Seblewonge . . . | | |
| | COMBINATION OF IL-4/IL- | | |
| | 13 PATHWAY INHIBITORS | | |
| | AND PLASMA CELL | | |
| | ABLATION FOR TREATING | | |
| | ALLERGY | | |
| | 2019 US20200024356 | | |
| | Smith, Eric; Olso . . . | | |
| | Bispecific Anti-BCMA × | | |
| | Anti-CD3 Antibodies and | | |
| | Uses Thereof | | |
| | 2019 WO2020018820 | | |
| | SMITH, Eric, OLSO . . . | | |
| | BISPECIFIC ANTI-BCMA × | | |
| | ANTI-CD3 ANTIBODIES | | |
| | AND USES THEREOF | | |
| | 2019 WO2020018825 | | |
| | DILILLO, David, D . . . | | |
| | CHIMERIC ANTIGEN | | |
| | RECEPTORS WITH BCMA | | |
| | SPECIFICITY AND USES | | |
| | THEREOF | | |
| | 2019 U.S. Pat. No. 11,384,153 Smith, | | |
| | Eric (New . . . Bispecific | | |
| | anti-BCMA × anti-CD3 | | |
| | antibodies and uses | | |
| | thereof | | |
| RO7297089 | 2019 US20200109202 | 2020 NCT04434469 Phase | Affimed Genentech |
| | Tesar, Michael; E . . . NK | 1 A Study Evaluating The | |
| | CELL ENGAGING | Safety And | |
| | ANTIBODY FUSION | Pharmacokinetics Of | |
| | CONSTRUCTS | Escalating Doses Of | |
| | 2019 WO2019198051 | RO7297089 In Patients | |
| | TESAR, Michael, E . . . NK | With Relapsed Or | |
| | CELL ENGAGING | Refractory Multiple | |
| | ANTIBODY FUSION | Myeloma | |
| | CONSTRUCTS | 2023 Plesner T, Harris . . . | |
| | | Phase I Study of Safety and | |
| | | Pharmacokinetics of | |
| | | RO7297089, an Anti- | |
| | | BCMA/CD16a Bispecific | |
| | | Antibody, in Patients with | |
| | | Relapsed, Refractory | |
| | | Multiple Myeloma. | |
| | | 2022 Cai H, Kakiuchi-K . . . | |
| | | Nonclinical | |
| | | Pharmacokinetics, | |
| | | Pharmacodynamics, and | |
| | | Translational Model of | |
| | | RO7297089, A Novel Anti- | |
| | | BCMA/CD16A Bispecific | |
| | | Tetravalent Antibody for | |
| | | the Treatment of Multiple | |
| | | Myeloma. | |
| | | ASH 2017 AFM26 - | |
| | | Targeting B Cell Maturation | |
| | | Antigen (BCMA) for NK | |
| | | Cell-Mediated | |
| | | Immunotherapy of | |
| | | Multiple Myeloma | |
| | | Thorsten Gantke, . . . | |
| Sichuan Kelun patent | 2021 WO2022007650 | | Sichuan Kelun Pharma |
| anti-BCMA / CD19 CAR | CHANG, Jianhui, Z . . . | | |
| | CHIMERIC ANTIGEN | | |
| | RECEPTOR CAR OR CAR | | |
| | CONSTRUCT TARGETING | | |
| | BCMA AND CD19 AND | | |
| | APPLICATION THEREOF | | |
| teclistamab | 2022 US20220411525 | 2022 NCT05338775 Phase | Janssen Biotech |
| | Adams, III, Homer . . . | 1 A Study of Talquetamab | |
| | BCMA AS A TARGET FOR | and Teclistamab Each in | |
| | T CELL REDIRECTING | Combination With a | |
| | ANTIBODIES IN B CELL | Programmed Cell Death | |
| | LYMPHOMAS | Receptor-1 (PD-1) Inhibitor | |
| | 2021 US20220041742 | for the Treatment of | |
| | Adams, Homer; Ban . . . | Participants With Relapsed | |
| | Methods for Treating | or Refractory Multiple | |
| | Multiple Myeloma | Myeloma | |
| | 2021 WO2021228783 | 2021 NCT04696809 Phase | |
| | ADAMS, Homer, GOL . . . | 1 A Study of Teclistamab in | |
| | METHODS FOR TREATING | Japanese Participants With | |
| | MULTIPLE MYELOMA | Relapsed or Refractory | |
| | 2019 WO2019220368 | Multiple Myeloma | |
| | ADAMS, Homer, GAU . . . | 2020 NCT04586426 Phase | |
| | BCMA/CD3 AND | 1 A Study of the | |
| | GPRDC5D/CD3 BISPECIFIC | Combination of | |
| | ANTIBODIES FOR USE IN | Talquetamab and | |
| | CANCER THERAPY | Teclistamab in Participants | |
| | 2016 US20170051068 | With Relapsed or | |
| | Pillarisetti, Kod . . . Anti- | Refractory Multiple | |
| | BCMA Antibodies, | Myeloma | |
| | Bispecific Antigen Binding | 2020 NCT04557098 Phase | |
| | Molecules that Bind | 2 A Study of Teclistamab, in | |
| | BCMA and CD3, and Uses | Participants With Relapsed | |
| | Thereof | or Refractory Multiple | |
| | 2016 WO2017031104 | Myeloma | |
| | PILLARISETTI, Kod . . . ANTI- | 2019 NCT04108195 Phase | |
| | BCMA ANTIBODIES, | 1 A Study of Subcutaneous | |
| | BISPECIFIC ANTIGEN | Daratumumab Regimens in | |
| | BINDING MOLECULES | Combination With | |
| | THAT BIND BCMA AND | Bispecific T Cell Redirection | |
| | CD3, AND USES THEREOF | Antibodies for the | |
| | 2016 U.S. Pat. No. 10,072,088 | Treatment of Participants | |
| | Pillarisetti, Kod . . . Anti- | With Multiple Myeloma | |
| | BCMA antibodies and | 2017 NCT03145181 Phase | |
| | uses thereof | 1 Dose Escalation Study of | |
| | | JNJ-64007957, a | |
| | | Humanized BCMA CD3 | |
| | | DuoBody ® Antibody, in | |
| | | Participants With Relapsed | |
| | | or Refractory Multiple | |
| | | Myeloma | |
| | | 2022 Kang C Teclistamab: | |
| | | First Approval. | |
| | | 2022 Hindié E Teclistamab | |
| | | in Relapsed or Refractory | |
| | | Multiple Myeloma. | |
| | | 2022 Girgis S, Wang Li . . . | |
| | | Effects of teclistamab and | |
| | | talquetamab on soluble | |
| | | BCMA levels in patients | |
| | | with relapsed/refractory | |
| | | multiple myeloma. | |
| | | 2022 Girgis S, Lin SXW . . . | |
| | | Translational Modeling | |
| | | Predicts Efficacious | |
| | | Therapeutic Dosing Range | |
| | | of Teclistamab for Multiple | |
| | | Myeloma. | |
| | | 2022 Moreau P, Garfall . . . | |
| | | Teclistamab in Relapsed or | |
| | | Refractory Multiple | |
| | | Myeloma. | |
| | | 2021 A BCMA-Targeted | |
| | | Bispecific Antibody Is | |
| | | Active in Multiple | |
| | | Myeloma. | |
| | | 2021 Usmani SZ, Garfal . . . | |
| | | Teclistamab, a B-cell | |
| | | maturation antigen × CD3 | |
| | | bispecific antibody, in | |
| | | patients with relapsed or | |
| | | refractory multiple | |
| | | myeloma (MajesTEC-1): a | |
| | | multicentre, open-label, | |
| | | single-arm, phase 1 study. | |
| | | 2020 Pillarisetti K, P . . . | |
| | | Teclistamab is an active T | |
| | | cell-redirecting bispecific | |
| | | antibody against B-cell | |
| | | maturation antigen for | |
| | | multiple myeloma. | |
| | | 2020 Frerichs KA, Broe . . . | |
| | | Preclinical activity of JNJ- | |
| | | 7957, a novel BCMA × CD3 | |
| | | bispecific antibody for the | |
| | | treatment of multiple | |
| | | myeloma, is potentiated by | |
| | | daratumumab. | |
| Tianjin Nankai Hosp. | | Xiong M, Liu R, L . . . A Novel | Tianjin Nankai Hosp. |
| anti-BCMA / CD3 | | CD3/BCMA Bispecific T-cell | |
| | | Redirecting Antibody for | |
| | | the Treatment of Multiple | |
| | | Myeloma. | |
| TNB-383B | 2021 US20220025047 | 2020 NCT04453397 | Abbvie TeneoBio |
| | Trinklein, Nathan . . . CD3 | Expanded Access for TNB- | |
| | BINDING ANTIBODIES | 383B in a Subject With | |
| | 2021 U.S. Pat. No. 11,421,027 | Relapsed/Refractory | |
| | Trinklein, Nathan . . . CD3 | Multiple Myeloma | |
| | binding antibodies | 2019 NCT03933735 Phase | |
| | 2021 WO2022006316 | 1 A Study of TNB-383B in | |
| | TRINKLEIN, Nathan . . . | Subjects With Relapsed or | |
| | MULTI-SPECIFIC | Refractory Multiple | |
| | ANTIBODIES BINDING TO | Myeloma | |
| | BCMA | 2022 D'Souza A, Shah N . . . A | |
| | 2021 US20210340255 | Phase I First-in-Human | |
| | Harris, Katherine . . . | Study of ABBV-383, a B-Cell | |
| | MULTISPECIFIC HEAVY | Maturation Antigen × CD3 | |
| | CHAIN ANTIBODIES WITH | Bispecific T-Cell Redirecting | |
| | MODIFIED HEAVY CHAIN | Antibody, in Patients With | |
| | CONSTANT REGIONS | Relapsed/Refractory | |
| | 2021 U.S. Pat. No. 11,186,639 Harris, | Multiple Myeloma. | |
| | Katherine . . . Multispecific | ASH 2017 T Cell | |
| | heavy chain antibodies | Engagement without | |
| | with modified heavy | Cytokine Storm: A Novel | |
| | chain constant regions | BCMA × CD3 Antibody | |
| | 2021 WO2021222578 | Killing Myeloma Cells with | |
| | HARRIS, Katherine . . . | Minimal Cytokine Secretion | |
| | MULTISPECIFIC HEAVY | Ben Buelow, MD, P . . . | |
| | CHAIN ANTIBODIES WITH | | |
| | MODIFIED HEAVY CHAIN | | |
| | CONSTANT REGIONS | | |
| | 2021 WO2021222616 | | |
| | BUELOW, Ben, SCHE . . . | | |
| | METHODS OF TREATING | | |
| | MULTIPLE MYELOMA | | |
| | 2021 US20210403587 | | |
| | Buelow, Ben; Sche . . . | | |
| | METHODS OF TREATING | | |
| | MULTIPLE MYELOMA | | |
| | 2019 WO2020061478 | | |
| | JORGENSEN, Brett, . . . | | |
| | METHODS FOR | | |
| | PURIFYING | | |
| | HETERODIMERIC | | |
| | MULTISPECIFIC | | |
| | ANTIBODIES | | |
| | 2018 WO2019133761 | | |
| | SCHELLENBERGER, U . . . | | |
| | CD3-DELTA/EPSILON | | |
| | HETERODIMER SPECIFIC | | |
| | ANTIBODIES | | |
| | 2018 US20200339685 | | |
| | Schellenberger, U . . . CD3- | | |
| | DELTA/EPSILON | | |
| | HETERODIMER SPECIFIC | | |
| | ANTIBODIES | | |
| | 2018 WO2019006072 | | |
| | KOCHENDERFER, Jam . . . | | |
| | ANTI-B-CELL | | |
| | MATURATION ANTIGEN | | |
| | CHIMERIC ANTIGEN | | |
| | RECEPTORS WITH | | |
| | HUMAN DOMAINS | | |
| | 2018 WO2018237006 | | |
| | TRINKLEIN, Nathan . . . | | |
| | ANTI-BCMA HEAVY | | |
| | CHAIN-ONLY ANTIBODIES | | |
| | 2018 WO2018237037 | | |
| | TRINKLEIN, Nathan . . . | | |
| | ANTI-BCMA HEAVY | | |
| | CHAIN-ONLY ANTIBODIES | | |
| | 2018 US20200157232 | | |
| | Trinklein, Nathan . . . ANTI- | | |
| | BCMA HEAVY CHAIN- | | |
| | ONLY ANTIBODIES | | |
| | 2018 US20210147564 | | |
| | Trinklein, Nathan . . . ANTI- | | |
| | BCMA HEAVY CHAIN- | | |
| | ONLY ANTIBODIES | | |
| | 2018 U.S. Pat. No. 11,427,642 | | |
| | Trinklein, Nathan . . . Anti- | | |
| | BCMA heavy chain-only | | |
| | antibodies | | |
| | 2017 WO2018119215 | | |
| | ALDRED, Shelley F . . . | | |
| | ANTI-BCMA HEAVY | | |
| | CHAIN-ONLY ANTIBODIES | | |
| | 2017 US20190352412 | | |
| | Force Aldred, She . . . ANTI- | | |
| | BCMA HEAVY CHAIN- | | |
| | ONLY ANTIBODIES | | |
| | 2017 U.S. Pat. No. 11,434,299 Force | | |
| | Aldred, She . . . Anti-BCMA | | |
| | heavy chain-only | | |
| | antibodies | | |
| | 2017 WO2017223111 | | |
| | TRINKLEIN, Nathan . . . CD3 | | |
| | BINDING ANTIBODIES | | |
| | 2017 WO2018052503 | | |
| | TRINKLEIN, Nathan . . . CD3 | | |
| | BINDING ANTIBODIES | | |
| | 2017 US20190263904 | | |
| | Trinklein, Nathan . . . CD3 | | |
| | BINDING ANTIBODIES | | |
| | 2017 US20200048348 | | |
| | Trinklein, Nathan . . . CD3 | | |
| | BINDING ANTIBODIES | | |
| | 2017 U.S. Pat. No. 11,505,606 | | |
| | Trinklein, Nathan . . . CD3 | | |
| | binding antibodies | | |
| TQB2934 | 2022 WO2022218380 | 2023 NCT05646758 Phase | Chia Tai Tianqing Pharma |
| | ZHANG, Bing MULTI- | 1 A Clinical Trial of | |
| | SPECIFIC ANTIBODY | TQB2934 for Injection in | |
| | TARGETING BCMA | Multiple Myeloma Subjects | |
| U. Florida patent anti- | 2016 WO2016130598 | | U. Florida |
| Syndecan-1 / BCMA CAR | CHANG, Lung-Ji BI- | | |
| | SPECIFIC CHIMERIC | | |
| | ANTIGEN RECEPTOR AND | | |
| | USES THEREOF | | |
| UCL patent anti-BCMA / | 2014 WO2015052536 | | UCL |
| APRIL | PULÉ, Martin, YON . . . | | |
| | MOLECULE | | |
| Virtuoso patent anti- | 2021 WO2021229306 | | Virtuoso |
| CD38 / BCMA | CHEN, Xiaocheng, . . . | | |
| | MULTISPECIFIC | | |
| | ANTIBODIES TARGETING | | |
| | CD38 AND BCMA AND | | |
| | USES THEREOF | | |
| Zymogenetics patent | 2011 US20120034159 | | Zymogenetics |
| anti-BCMA/TACI | KINDSVOGEL, WAYNE | | |
| | ANTIBODIES THAT BIND | | |
| | BOTH BCMA AND TACI | | |
| | 2009 U.S. Pat. No. 9,441,034 | | |
| | Sivakumar, Pallav . . . | | |
| | Compositions and | | |
| | methods for inhibiting | | |
| | PDGFRβ and VEGF-A | | |
| | 2002 WO2002066516 | | |
| | KINDSVOGEL, Wayne; | | |
| | ANTIBODIES THAT BIND | | |
| | BOTH BCMA AND TACI | | |
| Adimab patent anti- | | | Adimab |
| NKG2D / CD16 / BCMA | | | |
| Dragonfly patent anti- | 2021 US20220089760 | | Dragonfly |
| NKG2D / CD16 / BCMA | Bigelow, Mitchell . . . | | |
| | MULTI-SPECIFIC BINDING | | |
| | PROTEINS THAT BIND | | |
| | BCMA, NKG2D AND | | |
| | CD16, AND METHODS OF | | |
| | USE | | |
| | 2019 US20210317223 | | |
| | Bigelow, Mitchell . . . | | |
| | MULTI-SPECIFIC BINDING | | |
| | PROTEINS THAT BIND | | |
| | BCMA, NKG2D AND | | |
| | CD16, AND METHODS OF | | |
| | USE | | |
| HPN217 | 2022 WO2022256499 | 2020 NCT04184050 Phase | Abbvie Harpoon |
| | WESCHE, Holger BCMA | 1/Phase 2 A Phase 1/2 | |
| | TARGETING TRISPECIFIC | Open-label, Multicenter, | |
| | PROTEINS AND METHODS | Dose Escalation and Dose | |
| | OF USE | Expansion Study of the | |
| | 2021 US20210171649 | Safety, Tolerability, and PK | |
| | WESCHE, Holger; L . . . B | of HPN217 in Patients With | |
| | CELL MATURATION | R/R MM | |
| | ANTIGEN BINDING | | |
| | PROTEINS | | |
| | 2018 WO2019075378 | | |
| | WESCHE, Holger, L . . . B | | |
| | CELL MATURATION | | |
| | ANTIGEN BINDING | | |
| | PROTEINS | | |
| | 2018 WO2019075359 | | |
| | WESCHE, Holger, L . . . | | |
| | TRISPECIFIC PROTEINS | | |
| | AND METHODS OF USE | | |
| | 2018 US20190135930 | | |
| | Wesche, Holger; L . . . B | | |
| | CELL MATURATION | | |
| | ANTIGEN BINDING | | |
| | PROTEINS | | |
| | 2018 U.S. Pat. No. 10,927,180 | | |
| | Wesche, Holger (S . . . B cell | | |
| | maturation antigen | | |
| | binding proteins | | |
| Hummingbird patent | 2021 WO2022090556 | | Hummingbird Bio |
| anti-CD47 / BCMA/TACI | DECAILLOT, Fabien . . . | | |
| | BCMA/TACI ANTIGEN- | | |
| | BINDING MOLECULES | | |
| | 2018 WO2019110209 | | |
| | BOYD-KIRKUP, Jero . . . | | |
| | CD47 AND BCMA/TACI | | |
| | ANTIGEN-BINDING | | |
| | MOLECULES | | |
| Innovent patent anti- | 2022 WO2022174813 XU, | | Innovent |
| GPRC5D / BCMA / CD3 | Wei, SHEN, Wa . . . ANTI- | | |
| | GPRC5D × BCMA × CD3 | | |
| | TRISPECIFIC ANTIBODY | | |
| | AND USE THEREOF | | |
| Janssen patent anti- | 2022 US20220267438 | | Janssen Biotech |
| BCMA / GPRC5D / CD3 | Attar, Ricardo Ma . . . | | |
| | TRISPECIFIC ANTIBODY | | |
| | TARGETING BCMA, | | |
| | GPRC5D, AND CD3 | | |
| | 2022 WO2022175255 | | |
| | ATTAR, Ricardo Ma . . . | | |
| | TRISPECIFIC ANTIBODY | | |
| | TARGETING BCMA, | | |
| | GPRC5D, AND CD3 | | |

e. GPRC5D CAR

In some embodiments, the CAR is a GPRC5D CAR (“GPRC5D-CAR”), and in these embodiments, the polycistronic vector comprises an expression cassette that contains a nucleotide sequence encoding a GPRC5D CAR. GPRC5D is highly expressed on multiple myeloma cells and associated with poor prognostic factors. In some embodiments, the GPRC5D CAR may comprise a signal peptide, an extracellular binding domain that specifically binds GPRC5D, a hinge domain, a transmembrane domain, an intracellular costimulatory domain, and/or an intracellular signaling domain in tandem.

In some embodiments, the signal peptide of the GPRC5D CAR comprises a CD8a signal peptide. In some embodiments, the CD8a signal peptide comprises or consists of an amino acid sequence set forth in SEQ ID NO:6 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:6. In some embodiments, the signal peptide comprises an IgK signal peptide. In some embodiments, the IgK signal peptide comprises or consists of an amino acid sequence set forth in SEQ ID NO:7 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:7. In some embodiments, the signal peptide comprises a GMCSFR-α or CSF2RA signal peptide. In some embodiments, the GMCSFR-α or CSF2RA signal peptide comprises or consists of an amino acid sequence set forth in SEQ ID NO:8 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:8.

In some embodiments, the extracellular binding domain of the GPRC5D CAR is specific to GPRC5D, for example, human GPRC5D. The extracellular binding domain of the GPRC5D CAR can be codon-optimized for expression in a host cell or to have variant sequences to increase functions of the extracellular binding domain. In some embodiments, the extracellular binding domain comprises an immunogenically active portion of an immunoglobulin molecule, for example, an scFv.

In some embodiments, the extracellular binding domain of the GPRC5D CAR is derived from an antibody specific to GPRC5D, including, for example, any of the antibodies or CARs disclosed in Table 20, the references cited in which are incorporated by reference in their entireties herein. In any of these embodiments, the extracellular binding domain of the GPRC5D CAR can comprise or consist of the V_{H}, the V_{L}, and/or one or more CDRs of any of the antibodies disclosed in Table 20.

In some embodiments, the extracellular binding domain of the GPRC5D CAR comprises an scFv derived from the any of the antibodies or CARs disclosed in Table 20, optionally comprising the heavy chain variable region (V_{H}) and the light chain variable region (V_{L}) of one of the antibodies or CARs, connected by a linker. In some embodiments, the linker is a 3×G₄S linker. In other embodiments, the Whitlow linker may be used instead. In some embodiments, the GPRC5D-specific scFv comprises or consists of the scFv of an antibody or CAR disclosed in Table 20, or an amino acid sequence that is at least 80% identical (e.g., at least 80%, at least 85%, at least 90%, at least 95%, at least 96%, at least 97%, at least 98%, at least 99%, or 100% identical) to the amino acid sequence of the scFv of an antibody or CAR disclosed in Table 20. In some embodiments, the GPRC5D-specific scFv may comprise one or more CDRs having amino acid sequences of the CDRs of an antibody or CAR disclosed in Table 20. In some embodiments, the GPRC5D-specific scFv may comprise a heavy chain with one or more CDRs having amino acid sequences of the CDRs of an antibody or CAR disclosed in Table 20. In some embodiments, the GPRC5D-specific scFv may comprise a light chain with one or more CDRs having amino acid sequences of the CDRs of an antibody or CAR disclosed in Table 20. In any of these embodiments, the GPRC5D-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the GPRC5D CAR comprises or consists of the one or more CDRs as described herein, including in Table 20.

TABLE 20
Exemplary GPRC5D antigen binding domains

| Ab/CAR | | | |
|---|---|---|---|
| Name | Antigen | Company | Reference |
| | GPRC5D | Sloan-Kettering, | WO2016/090312 |
| | | Eureka | |
| | GPRC5D | Daiichi Sankyo | WO2018/147245, |
| | | | US20190367612 |
| | GPRC5D | Eureka, Sloan- | U.S. Pat. No. |
| | | Kettering | 10,590,196 |
| | GPRC5D | Janssen Biotech | US20200231686 |
| | GPRC5D | Juno, Sloan-Kettering | US20210393689 |
| | GPRC5D | Roche | US20210054094 |
| | GPRC5D, CD3 | Chugai | WO2022/025220 |
| Talquetamab | GPRC5D, CD3 | Genmab, Janssen | US20180037651 |
| | | Biotech | |

In some embodiments, the hinge domain of the GPRC5D CAR comprises a CD8a hinge domain, for example, a human CD8a hinge domain. In some embodiments, the CD8a hinge domain comprises or consists of an amino acid sequence set forth in SEQ ID NO:9 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:9. In some embodiments, the hinge domain comprises a CD28 hinge domain, for example, a human CD28 hinge domain. In some embodiments, the CD28 hinge domain comprises or consists of an amino acid sequence set forth in SEQ ID NO:10 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:10. In some embodiments, the hinge domain comprises an IgG4 hinge domain, for example, a human IgG4 hinge domain. In some embodiments, the IgG4 hinge domain comprises or consists of an amino acid sequence set forth in SEQ ID NO:11 or SEQ ID NO:12, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:11 or SEQ ID NO:12. In some embodiments, the hinge domain comprises a IgG4 hinge-Ch2-Ch3 domain, for example, a human IgG4 hinge-Ch2-Ch3 domain. In some embodiments, the IgG4 hinge-Ch2-Ch3 domain comprises or consists of an amino acid sequence set forth in SEQ ID NO:13 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:13.

In some embodiments, the transmembrane domain of the GPRC5D CAR comprises a CD8α transmembrane domain, for example, a human CD8α transmembrane domain. In some embodiments, the CD8α transmembrane domain comprises or consists of an amino acid sequence set forth in SEQ ID NO:14 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:14. In some embodiments, the transmembrane domain comprises a CD28 transmembrane domain, for example, a human CD28 transmembrane domain. In some embodiments, the CD28 transmembrane domain comprises or consists of an amino acid sequence set forth in SEQ ID NO:15 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:15.

In some embodiments, the intracellular costimulatory domain of the GPRC5D CAR comprises a 4-1BB costimulatory domain, for example, a human 4-1BB costimulatory domain. In some embodiments, the 4-1BB costimulatory domain comprises or consists of an amino acid sequence set forth in SEQ ID NO:16 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:16. In some embodiments, the intracellular costimulatory domain comprises a CD28 costimulatory domain, for example, a human CD28 costimulatory domain. In some embodiments, the CD28 costimulatory domain comprises or consists of an amino acid sequence set forth in SEQ ID NO:17 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:17.

In some embodiments, the intracellular signaling domain of the GPRC5D CAR comprises a CD3 zeta (ζ) signaling domain, for example, a human CD3ζ signaling domain. In some embodiments, the CD3ζ signaling domain comprises or consists of an amino acid sequence set forth in SEQ ID NO:18 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:18.

In some embodiments, the polycistronic vector comprises an expression cassette that contains a nucleotide sequence encoding a GPRC5D CAR, including, for example, a GPRC5D CAR comprising the GPRC5D-specific scFv having sequences of an antibody or CAR disclosed in Table 20, the CD8α hinge domain of SEQ ID NO:9, the CD8α transmembrane domain of SEQ ID NO:14, the 4-1BB costimulatory domain of SEQ ID NO:16, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a GPRC5D CAR, including, for example, a GPRC5D CAR comprising the GPRC5D-specific scFv having sequences of an antibody or CAR disclosed in Table 20, the CD28 hinge domain of SEQ ID NO:10, the CD8α transmembrane domain of SEQ ID NO:14, the 4-1BB costimulatory domain of SEQ ID NO:16, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a GPRC5D CAR, including, for example, a GPRC5D CAR comprising the GPRC5D-specific scFv having sequences of an antibody or CAR disclosed in Table 20, the IgG4 hinge domain of SEQ ID NO:11 134 or SEQ ID NO:12, the CD8α transmembrane domain of SEQ ID NO:14, the 4-1BB costimulatory domain of SEQ ID NO:16, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a GPRC5D CAR, including, for example, a GPRC5D CAR comprising the GPRC5D-specific scFv having sequences of an antibody or CAR disclosed in Table 20, the CD8α hinge domain of SEQ ID NO:9, the CD28 transmembrane domain of SEQ ID NO:15, the 4-1BB costimulatory domain of SEQ ID NO:16, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a GPRC5D CAR, including, for example, a GPRC5D CAR comprising the GPRC5D-specific scFv having sequences of an antibody or CAR disclosed in Table 20, the CD28 hinge domain of SEQ ID NO:10, the CD28 transmembrane domain of SEQ ID NO:15, the 4-1BB costimulatory domain of SEQ ID NO:16, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a GPRC5D CAR, including, for example, a GPRC5D CAR comprising the GPRC5D-specific scFv having sequences of an antibody or CAR disclosed in Table 20, the IgG4 hinge domain of SEQ ID NO:11 or SEQ ID NO:12, the CD28 transmembrane domain of SEQ ID NO:15, the 4-1BB costimulatory domain of SEQ ID NO:16, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, a polynucleotide provided herein comprises a nucleotide sequence encoding a GPRC5D CAR, a variable domain of a GPRC5D CAR, or a set of CDRs (HCDR 1, 2, and 3 and LCDR 1, 2, and 3) that encodes a GPRC5D CAR as set forth in TABLE 21 below or a variable domain of a GPRC5D CAR or a set of CDRs (HCDR 1, 2, and 3 and LCDR 1, 2, and 3) therefrom. In some embodiments, a polynucleotide provided herein comprises a nucleotide sequence encoding a GPRC5D CAR, a variable domain of a GPRC5D CAR, or a set of CDRs (HCDR 1, 2, and 3 and LCDR 1, 2, and 3) that having at least 80% (e.g., at least 80%, at least 85%, at least 90%, at least 95%, at least 96%, at least 97%, at least 98%, at least 99%, or 100% identical) to a GPRC5D CAR as set forth in TABLE 21 below or a variable domain of a GPRC5D CAR or a set of CDRs (HCDR 1, 2, and 3 and LCDR 1, 2, and 3) therefrom, respectively.

TABLE 21
Exemplary GPRC5D antigen binding domains

| Antibody Name | Patents | Publications | Company |
|---|---|---|---|
| BRL Med. patent anti- | 2022 WO2022247756 | | BRL Med. |
| GPRC5D CAR | | | |
| Daiichi Sankyo patent | 2018 US20190367612 | | Daiichi Sankyo |
| anti-GPRC5D | 2018 WO2018147245 | | |
| Eureka patent anti- | 2019 US20200123249 | | Eureka and Sloan- |
| GPRC5D | 2019 US20200123250 | | Kettering |
| | 2019 U.S. Pat. No. 11,566,071 | | |
| | 2017 US20180118822 | | |
| | 2017 U.S. Pat. No. 10,590,196 | | |
| | 2015 WO2016090329 | | |
| Janssen patent anti- | 2021 WO2022013819 | | Janssen Biotech |
| GPRC5D CAR | 2021 US20220064284 | | |
| | 2020 WO2020148677 | | |
| Juno patent anti- | 2019 WO2020092854 | | Juno and Sloan- |
| GPRC5D CAR | 2019 US20210393689 | | Kettering |
| Lanova patent anti- | 2022 US20220220203 | | Lanova |
| GPRC5D | 2022 U.S. Pat. No. 11,485,783 | | |
| | 2022 WO2022148370 | | |
| Nanjing Bioheng Bio | 2022 WO2022222910 | | Nanjing Bioheng Bio |
| patent anti-GPRC5D | | | |
| Roche patent anti- | 2022 US20220259318 | | Roche |
| GPRC5D | 2022 US20220411491 | | |
| | 2020 US20210054094 | | |
| | 2020 WO2021018925 | | |
| | 2020 WO2021018859 | | |
| | 2019 WO2019154890 | | |
| Shanghai Symray Bio | 2022 WO2022247804 | | Shanghai Symray Bio |
| patent anti-GPRC5D | | | |
| Chugai anti-GPRC5D/ | | 2019 Kodama T, Kochi | Chugai |
| CD3 | | Y . . . Anti-GPRC5D/CD3 | |
| | | bispecific T cell- | |
| | | redirecting antibody | |
| | | for the treatment of | |
| | | multiple myeloma. | |
| talquetamab | 2021 WO2022058445 | 2022 NCT05461209 | Genmab Janssen |
| | 2021 US20220177584 | Phase 3 A Study of | Biotech |
| | 2019 WO2019220368 | Comparing | |
| | 2017 WO2018017786 | Talquetamab to | |
| | 2017 US20180037651 | Belantamab Mafodotin | |
| | 2017 U.S. Pat. No. 10,562,968 | in Participants With | |
| | | Relapsed/Refractory | |
| | | Multiple Myeloma | |
| | | 2022 NCT05338775 | |
| | | Phase 1 A Study of | |
| | | Talquetamab and | |
| | | Teclistamab Each in | |
| | | Combination With a | |
| | | Programmed Cell | |
| | | Death Receptor-1 (PD- | |
| | | 1) Inhibitor for the | |
| | | Treatment of | |
| | | Participants With | |
| | | Relapsed or Refractory | |
| | | Multiple Myeloma | |
| | | 2021 NCT04773522 | |
| | | Phase 1 A Study of JNJ- | |
| | | 64407564 in Japanese | |
| | | Participants With | |
| | | Relapsed or Refractory | |
| | | Multiple Myeloma | |
| | | 2021 NCT04634552 | |
| | | Phase 2 A Study of | |
| | | Talquetamab in | |
| | | Participants With | |
| | | Relapsed or Refractory | |
| | | Multiple Myeloma | |
| | | 2020 NCT04586426 | |
| | | Phase 1 A Study of the | |
| | | Combination of | |
| | | Talquetamab and | |
| | | Teclistamab in | |
| | | Participants With | |
| | | Relapsed or Refractory | |
| | | Multiple Myeloma | |
| | | 2019 NCT04108195 | |
| | | Phase 1 A Study of | |
| | | Subcutaneous | |
| | | Daratumumab | |
| | | Regimens in | |
| | | Combination With | |
| | | Bispecific T Cell | |
| | | Redirection Antibodies | |
| | | for the Treatment of | |
| | | Participants With | |
| | | Multiple Myeloma | |
| | | 2017 NCT03399799 | |
| | | Phase 1 Dose | |
| | | Escalation Study of JNJ- | |
| | | 64407564 in | |
| | | Participants With | |
| | | Relapsed or Refractory | |
| | | Multiple Myeloma | |
| | | 2023 MonumenTAL | |
| | | Results for | |
| | | Talquetamab in | |
| | | Myeloma. | |
| | | 2022 Chari A, Minnema | |
| | | . . . Talquetamab, a T- | |
| | | Cell-Redirecting | |
| | | GPRC5D Bispecific | |
| | | Antibody for Multiple | |
| | | Myeloma. | |
| | | 2022 Narayan N, | |
| | | Willia . . . | |
| | | Onychomadesis and | |
| | | palmoplantar | |
| | | keratoderma | |
| | | associated with | |
| | | talquetamab therapy | |
| | | for relapsed and | |
| | | refractory multiple | |
| | | myeloma. | |
| | | 2022 Girgis S, Wang | |
| | | Li . . . Effects of | |
| | | teclistamab and | |
| | | talquetamab on soluble | |
| | | BCMA levels in patients | |
| | | with | |
| | | relapsed/refractory | |
| | | multiple myeloma. | |
| | | 2021 Verkleij CPM, | |
| | | Bro . . . Preclinical | |
| | | activity and | |
| | | determinants of | |
| | | response of the | |
| | | GPRC5D × CD3 bispecific | |
| | | antibody talquetamab | |
| | | in multiple myeloma. | |
| | | 2020 Pillarisetti K, E . . . A | |
| | | T-cell-redirecting | |
| | | bispecific G-protein- | |
| | | coupled receptor class | |
| | | 5 member D × CD3 | |
| | | antibody to treat | |
| | | multiple myeloma. | |
| Innovent patent anti- | 2022 WO2022174813 | | Innovent |
| GPRC5D/BCMA/CD3 | | | |
| Janssen patent anti- | 2022 US20220267438 | | Janssen Biotech |
| BCMA/GPRC5D/CD3 | 2022 WO2022175255 | | |

f. CD38 CAR

In some embodiments, the CAR is a CD38 CAR (“CD38-CAR”), and in these embodiments, the polycistronic vector comprises an expression cassette that contains a nucleotide sequence encoding a CD38 CAR. CD38 is highly expressed on multiple myeloma cells. In some embodiments, the CD38 CAR may comprise a signal peptide, an extracellular binding domain that specifically binds CD38, a hinge domain, a transmembrane domain, an intracellular costimulatory domain, and/or an intracellular signaling domain in tandem.

In some embodiments, the signal peptide of the CD38 CAR comprises a CD8α signal peptide. In some embodiments, the CD8α signal peptide comprises or consists of an amino acid sequence set forth in SEQ ID NO:6 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:6. In some embodiments, the signal peptide comprises an IgK signal peptide. In some embodiments, the IgK signal peptide comprises or consists of an amino acid sequence set forth in SEQ ID NO:7 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:7. In some embodiments, the signal peptide comprises a GMCSFR-α or CSF2RA signal peptide. In some embodiments, the GMCSFR-α or CSF2RA signal peptide comprises or consists of an amino acid sequence set forth in SEQ ID NO:8 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:8.

In some embodiments, the extracellular binding domain of the CD38 CAR is specific to CD38, for example, human CD38. The extracellular binding domain of the GPRC5D CAR can be codon-optimized for expression in a host cell or to have variant sequences to increase functions of the extracellular binding domain. In some embodiments, the extracellular binding domain comprises an immunogenically active portion of an immunoglobulin molecule, for example, an scFv.

In some embodiments, the extracellular binding domain of the CD38 CAR is derived from an antibody specific to CD38, including, for example, any of the antibodies or CARs disclosed in Table 22, the references cited in which are incorporated by reference in their entireties herein. In any of these embodiments, the extracellular binding domain of the CD38 CAR can comprise or consist of the V_{H}, the V_{L}, and/or one or more CDRs of any of the antibodies in Table 22.

In some embodiments, the extracellular binding domain of the CD38 CAR comprises an scFv derived from the any of the antibodies or CARs disclosed in Table 22, optionally comprising the heavy chain variable region (V_{H}) and the light chain variable region (V_{L}) of one of the antibodies or CARs, connected by a linker. In some embodiments, the linker is a 3×G₄S linker. In other embodiments, the Whitlow linker may be used instead. In some embodiments, the CD38-specific scFv comprises or consists of the scFv of an antibody or CAR disclosed in Table 22, or an amino acid sequence that is at least 80% identical (e.g., at least 80%, at least 85%, at least 90%, at least 95%, at least 96%, at least 97%, at least 98%, at least 99%, or 100% identical) to the amino acid sequence of the scFv of an antibody or CAR disclosed in Table 22. In some embodiments, the CD38-specific scFv may comprise one or more CDRs having amino acid sequences of the CDRs of an antibody or CAR disclosed in Table 22. In some embodiments, the CD38-specific scFv may comprise a heavy chain with one or more CDRs having amino acid sequences of the CDRs of an antibody or CAR disclosed in Table 22. In some embodiments, the CD38-specific scFv may comprise a light chain with one or more CDRs having amino acid sequences of the CDRs of an antibody or CAR disclosed in Table 22. In any of these embodiments, the CD38-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the CD38 CAR comprises or consists of the one or more CDRs as described herein, including in Table 22.

TABLE 22
Exemplary CD38 antigen binding domains

| Ab/CAR Name | Antigen | Company | Reference |
|---|---|---|---|
| | CD38 | Centrose | U.S. Pat. No. 10,675,352 |
| | CD38 | Chengdu Conmed Bio | WO2021/104052 |
| CID-103 | CD38 | Black Belt, CASI, Tusk | U.S. Pat. No. 11,236,173 |
| Daratumumab | CD38 | Genmab Janssen Biotech | U.S. Pat. No. 9,187,565 |
| | CD38 | Encefa | WO2022/018229 |
| Felzartamab | CD38 | Celgene I-Mab | WO2017/218698 |
| | | Biopharma Morphosys | |
| GEN3014 | CD38 | Genmab | U.S. Pat. No. 9,187,565 |
| | CD38 | Genmab VU | U.S. Pat. No. 7,829,673 |
| | | U. Med. Center | |
| Isatuximab-irfc | CD38 | ImmunoGen Sanofi | U.S. Pat. No. 11,090,390 |
| | CD38 | Jiangsu Hengrui | WO2020/052546 |
| | CD38 | Jiangsu Kanion | WO2021/259227 |
| | CD38 | Jiangsu Simcere | WO2021/115404 |
| | CD38 | Kleo | WO2021/003050 |
| | CD38 | Lentigen | WO2020/113108 |
| | CD38 | Ligand | |
| Mezagitamab | CD38 | Takeda | U.S. Pat. No. 9,790,285 |
| | CD38 | Millennium | |
| | CD38 | Momenta | WO2021/055876 |
| OKT10-B10 | CD38 | FHCRC | US20210171651 |
| | CD38 | PeptiDream | WO2021/002265 |
| SAR442085 | CD38 | Sanofi | U.S. Pat. No. 8,153,765 |
| SG301 | CD38 | Hangzhou Sumgen | US20210024645 |
| | | Biotech | |
| | CD38 | Shanghai Puremab | WO2021/052465 |
| | CD38 | Sorrento | US20190135937 |
| STI-6129 | CD38 | Sorrento | U.S. Pat. No. 10,800,852 |
| | CD38 | Acroimmune Suzhou | US20210324102 |
| | | Stainwei Bio | |
| | CD38 | Teva | U.S. Pat. No. 10,981,986 |
| TNB-738 | CD38 | Ancora TeneoBio | |
| | CD38 | U. California | U.S. Pat. No. 10,865,249 |
| | CD38 | U. Liege | US20200393466 |
| | CD38 | UMC Utrecht | US20190233533 |
| | CD38 | Y-mAbs | WO2021/254574 |
| | CD38 | Yeda R&D | US20200384024 |
| AMG 424 | CD38 CD3 | Amgen Xencor | WO2017/091656 |
| | CD38 CD19 | BioGraph 55 | WO2021/173844 |
| BMX-101 | CD38 PD-L1 | Biomunex | US20200010559 |
| GBR 1342 | CD38 CD3 | Glenmark/Ichnos | WO2016/071355 |
| IGM-2644 | CD38 CD3 | IGM Bio | WO2015/153912 |
| | CD38 CD3 | INSERM | WO2021/009263 |
| | CD38 CD3 | Sorrento | WO2021/003189 |
| | CD38 BCMA | Virtuoso | WO2021/229306 |
| | CD38 ICAM-1 | U. California Virtuoso | US20220002432 |
| | CD38 CD3 | Xencor | US20160215063 |
| Y150 | CD38 CD3 | Wuhan YZY Biopharma | |
| SAR442257 | CD38 CD28 CD3 | Sanofi | US20200140552 |

In some embodiments, the hinge domain of the CD38 CAR comprises a CD8α hinge domain, for example, a human CD8α hinge domain. In some embodiments, the CD8α hinge domain comprises or consists of an amino acid sequence set forth in SEQ ID NO:9 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:9. In some embodiments, the hinge domain comprises a CD28 hinge domain, for example, a human CD28 hinge domain. In some embodiments, the CD28 hinge domain comprises or consists of an amino acid sequence set forth in SEQ ID NO:10 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:10. In some embodiments, the hinge domain comprises an IgG4 hinge domain, for example, a human IgG4 hinge domain. In some embodiments, the IgG4 hinge domain comprises or consists of an amino acid sequence set forth in SEQ ID NO:11 or SEQ ID NO:12, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:11 or SEQ ID NO:12. In some embodiments, the hinge domain comprises a IgG4 hinge-Ch2-Ch3 domain, for example, a human IgG4 hinge-Ch2-Ch3 domain. In some embodiments, the IgG4 hinge-Ch2-Ch3 domain comprises or consists of an amino acid sequence set forth in SEQ ID NO:13 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:13.

In some embodiments, the transmembrane domain of the CD38 CAR comprises a CD8α transmembrane domain, for example, a human CD8α transmembrane domain. In some embodiments, the CD8α transmembrane domain comprises or consists of an amino acid sequence set forth in SEQ ID NO:14 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:14. In some embodiments, the transmembrane domain comprises a CD28 transmembrane domain, for example, a human CD28 transmembrane domain. In some embodiments, the CD28 transmembrane domain comprises or consists of an amino acid sequence set forth in SEQ ID NO:15 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:15.

In some embodiments, the intracellular costimulatory domain of the CD38 CAR comprises a 4-1BB costimulatory domain, for example, a human 4-1BB costimulatory domain. In some embodiments, the 4-1BB costimulatory domain comprises or consists of an amino acid sequence set forth in SEQ ID NO:16 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:16. In some embodiments, the intracellular costimulatory domain comprises a CD28 costimulatory domain, for example, a human CD28 costimulatory domain. In some embodiments, the CD28 costimulatory domain comprises or consists of an amino acid sequence set forth in SEQ ID NO:17 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:17.

In some embodiments, the intracellular signaling domain of the CD38 CAR comprises a CD3 zeta (ζ) signaling domain, for example, a human CD3ζ signaling domain. In some embodiments, the CD3ζ signaling domain comprises or consists of an amino acid sequence set forth in SEQ ID NO:18 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:18.

In some embodiments, the polycistronic vector comprises an expression cassette that contains a nucleotide sequence encoding a CD38 CAR, including, for example, a CD38 CAR comprising the CD38-specific scFv having sequences of an antibody or CAR disclosed in Table 22, the CD8α hinge domain of SEQ ID NO:9, the CD8α transmembrane domain of SEQ ID NO:14, the 4-1BB costimulatory domain of SEQ ID NO:16, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD38 CAR, including, for example, a CD38 CAR comprising the CD38-specific scFv having sequences of an antibody or CAR disclosed in Table 22, the CD28 hinge domain of SEQ ID NO:10, the CD8α transmembrane domain of SEQ ID NO:14, the 4-1BB costimulatory domain of SEQ ID NO:16, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD38 CAR, including, for example, a CD38 CAR comprising the CD38-specific scFv having sequences of an antibody or CAR disclosed in Table 22, the IgG4 hinge domain of SEQ ID NO:11 134 or SEQ ID NO:12, the CD8α transmembrane domain of SEQ ID NO:14, the 4-1BB costimulatory domain of SEQ ID NO:16, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD38 CAR, including, for example, a CD38 CAR comprising the CD38-specific scFv having sequences of an antibody or CAR disclosed in Table 22, the CD8α hinge domain of SEQ ID NO:9, the CD28 transmembrane domain of SEQ ID NO:15, the 4-1BB costimulatory domain of SEQ ID NO:16, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD38 CAR, including, for example, a CD38 CAR comprising the CD38-specific scFv having sequences of an antibody or CAR disclosed in Table 22, the CD28 hinge domain of SEQ ID NO:10, the CD28 transmembrane domain of SEQ ID NO:15, the 4-1BB costimulatory domain of SEQ ID NO:16, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD38 CAR, including, for example, a CD38 CAR comprising the CD38-specific scFv having sequences of an antibody or CAR disclosed in Table 22, the IgG4 hinge domain of SEQ ID NO:11 or SEQ ID NO:12, the CD28 transmembrane domain of SEQ ID NO:15, the 4-1BB costimulatory domain of SEQ ID NO:16, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, a polynucleotide provided herein comprises a nucleotide sequence encoding a CD38 CAR, a variable domain of a CD38 CAR, or a set of CDRs (HCDR 1, 2, and 3 and LCDR 1, 2, and 3) that encodes a CD38 CAR as set forth in TABLE 23 below or a variable domain of a CD38 CAR or a set of CDRs (HCDR 1, 2, and 3 and LCDR 1, 2, and 3) therefrom. In some embodiments, a polynucleotide provided herein comprises a nucleotide sequence encoding a CD38 CAR, a variable domain of a CD38 CAR, or a set of CDRs (HCDR 1, 2, and 3 and LCDR 1, 2, and 3) that having at least 80% (e.g., at least 80%, at least 85%, at least 90%, at least 95%, at least 96%, at least 97%, at least 98%, at least 99%, or 100% identical) to a CD38 CAR as set forth in TABLE 23 below or a variable domain of a CD38 CAR or a set of CDRs (HCDR 1, 2, and 3 and LCDR 1, 2, and 3) therefrom, respectively.

TABLE 23
Exemplary CD38 antigen binding domains

| Antibody | | | |
|---|---|---|---|
| Name | Patents | Publications | Company |
| Centrose | 2015 WO2015123687 | | Centrose |
| patent anti- | PRUDENT, James, R. | | |
| CD38 | EXTRACELLULAR | | |
| | TARGETED DRUG | | |
| | CONJUGATES | | |
| Chengdu | 2020 WO2021104052 | | Chengdu |
| Conmed Bio | YU, Juntao, XU, G . . . | | Conmed Bio |
| patent anti- | PHARMACEUTICAL | | |
| CD38 | COMPOSITION, | | |
| | PREPARATION | | |
| | METHOD THEREFOR | | |
| | AND USE THEREOF | | |
| CID-103 | 2022 | 2021 NCT04758767 Phase 1 CID-103 (Anti- | Black Belt |
| | US20220135697 | CD38 Antibody) in Previously Treated | CASI Tusk |
| | GOUBIER, Anne; SA . . . | Relapsed or Refractory Multiple Myeloma | |
| | CD38 ANTIBODY | AACR 2018 A best in class anti-CD38 | |
| | 2018 WO2019034753 | antibody with antitumor and immune- | |
| | GOUBIER, Anne, SA . . . | modulatory properties N. Eissler, S. Fi | |
| | CD38 ANTIBODY | | |
| | 2018 WO2019034752 | | |
| | GOUBIER, Anne, SA . . . | | |
| | CD38 MODULATING | | |
| | ANTIBODY | | |
| | 2018 | | |
| | US20200362050 | | |
| | GOUBIER, Anne; SA . . . | | |
| | CD38 ANTIBODY | | |
| | 2018 | | |
| | US20200362049 | | |
| | GOUBIER, Anne; SA . . . | | |
| | CD38 MODULATING | | |
| | ANTIBODY | | |
| | 2018 U.S. Pat. No. 11,236,173 | | |
| | Goubier, Anne (St . . . | | |
| | CD38 antibody | | |
| | 2018 U.S. Pat. No. 11,542,338 | | |
| | Goubier, Anne; Sa . . . | | |
| | CD38 modulating | | |
| | antibody | | |
| | 2018 WO2018224683 | | |
| | MERCHIERS, Pascal . . . | | |
| | CD38 MODULATING | | |
| | ANTIBODY | | |
| | 2018 WO2018224682 | | |
| | MERCHIERS, Pascal . . . | | |
| | CD38 MODULATING | | |
| | ANTIBODY | | |
| | 2018 WO2018224685 | | |
| | MERCHIERS, Pascal . . . | | |
| | CD38 MODULATING | | |
| | ANTIBODY | | |
| | 2018 | | |
| | US20200190209 | | |
| | Merchiers, Pascal . . . | | |
| | CD38 MODULATING | | |
| | ANTIBODY | | |
| | 2018 | | |
| | US20210277138 | | |
| | Merchiers, Pascal . . . | | |
| | CD38 MODULATING | | |
| | ANTIBODY AGENTS | | |
| CM313 | | 2023 NCT05694767 Phase 2 A Prospective, | Keymed |
| | | One-arm and Open Clinical Study of | |
| | | CM313 in the Treatment of Immune | |
| | | Thrombocytopenia | |
| | | 2022 NCT05465707 Phase 1/Phase 2 A | |
| | | Study of CM313 Injection in Subjects With | |
| | | Systemic Lupus Erythematosus | |
| | | 2021 NCT04818372 Phase 1 Dose | |
| | | Escalation and Expansion Study of CM313 | |
| | | in Subjects With Relapsed or Refractory | |
| | | Multiple Myeloma and Lymphoma | |
| daratumumab | 2022 WO2022175920 | 2023 Habicht CP, Ridde . . . Mitigation of | Genmab |
| | ARIAS, Diane Alvarez | therapeutic anti-CD38 antibody | Janssen |
| | COMBINATION | interference with fab fragments: How well | Biotech |
| | THERAPIES WITH | does it perform? | |
| | ANTI-CD38 | 2022 Chen M, Zhao L, Y . . . Severe lung | |
| | ANTIBODIES AND | injury induced by CD38 monoclonal | |
| | PARP OR ADENOSINE | antibody Daratumumab and bortezomib- | |
| | RECEPTOR | containing regimen in a patient with | |
| | INHIBITORS | preexisting interstitial lung disease: a case | |
| | 2022 | report and literature review. | |
| | US20220275090 | 2022 Jeong IH, Seo JY, . . . Detection of | |
| | Alvarez Arias, Di . . . | unexpected antibodies in Korean multiple | |
| | Combination | myeloma patients on daratumumab using | |
| | Therapies with Anti- | dithiothreitol-treated reagent cells is more | |
| | CD38 Antibodies and | efficient than extended phenotyping and | |
| | PARP or Adenosine | genotyping. | |
| | Receptor Inhibitors | 2022 Djebbari F, Poynt . . . Outcomes of anti- | |
| | 2022 | CD38 isatuximab plus pomalidomide and | |
| | US20220275101 | dexamethasone in five relapsed myeloma | |
| | Schecter, Jordan; Use | patients with prior exposure to anti-C38 | |
| | of Approved Anti- | daratumumab: case series. | |
| | CD38 Antibody Drug | 2022 Liang S, Feng W, . . . False positive | |
| | Product to Treat Light | results: a challenge for laboratory | |
| | Chain Amyloidosis | physicians and hematologists in treating | |
| | 2021 | multiple myeloma with daratumumab. | |
| | US20220202859 | 2022 Wechalekar AD, Sa . . . Daratumumab | |
| | TERRETT, Jonathan . . . | in AL amyloidosis. | |
| | CANCER TREATMENT | 2022 Zhao AL, Tang WJ, . . . [Efficacy and | |
| | USING CD38 | safety of daratumumab in patients with | |
| | INHIBITOR AND/OR | relapsed/refractory multiple myeloma]. | |
| | LENALIDOMIDE AND | 2022 Jing H, Yang L, Q . . . Safety and efficacy | |
| | T-CELLS EXPRESSING | of daratumumab in Chinese patients with | |
| | A CHIMERIC ANTIGEN | relapsed or refractory multiple myeloma: a | |
| | RECEPTOR | phase 1, dose-escalation study | |
| | 2021 WO2022137186 | (MMY1003). | |
| | TERRETT, Jonathan . . . | 2022 Al-Attar A, Woda BA κ light chain- | |
| | CANCER TREATMENT | expressing hematogones in a patient with | |
| | USING CD38 | λ-restricted CLL and multiple myeloma on | |
| | INHIBITOR AND/OR | daratumumab therapy. | |
| | LENALIDOMIDE AND | 2022 Fu W, Li W, Hu J, . . . Daratumumab, | |
| | T-CELLS EXPRESSING | Bortezomib, and Dexamethasone versus | |
| | A CHIMERIC ANTIGEN | Bortezomib and Dexamethasone in | |
| | RECEPTOR | Chinese Patients With Relapsed or | |
| | 2021 | Refractory Multiple Myeloma: Updated | |
| | US20220204636 DE | Analysis of LEPUS. | |
| | WEERS, Michel; . . . | 2022 Chami B, Okuda M, . . . Anti-CD38 | |
| | ANTIBODIES AGAINST | monoclonal antibody interference with | |
| | HUMAN CD38 | blood compatibility testing: Differentiating | |
| | 2021 | isatuximab and daratumumab via | |
| | US20220062415 Xie, | functional epitope mapping. | |
| | Hong; Cakana . . . | 2022 Oyama T, Taoka K, . . . Daratumumab | |
| | Methods of Treating | plus lenalidomide and dexamethasone for | |
| | Multiple Myeloma | relapsed POEMS syndrome with bone | |
| | 2021 | plasmacytoma harboring 17p deletion. | |
| | US20220202937 | 2022 Oses L, Brulc E, . . . MM-315 Light- | |
| | Mackay, Joanna; | Chain Amyloidosis Patients Treated With | |
| | Novel Formulations | Daratumumab: A Single-Center | |
| | Which Stabilize Low | Experience. | |
| | Dose Antibody | 2022 Du C, Sui W, Huan . . . Effect of clinical | |
| | Compositions | application of anti-CD38 and anti-CD47 | |
| | 2021 | monoclonal antibodies on blood group | |
| | US20220041745 | detection and transfusion therapy and | |
| | Bandekar, Rajesh; . . . | treatment. | |
| | Clinically Proven | 2022 Taenaka R, Shimok . . . Successful | |
| | Subcutaneous | treatment with daratumumab, | |
| | Pharmaceutical | lenalidomide, and dexamethasone therapy | |
| | Compositions | followed by autologous stem cell | |
| | Comprising Anti- | transplantation for newly diagnosed | |
| | CD38 Antibodies and | polyneuropathy, organomegaly, | |
| | Their Uses | endocrinopathy, M-protein, skin changes | |
| | 2021 | syndrome: a case report. | |
| | US20220098318 | 2022 Leleu X, Martin T . . . Anti-CD38 | |
| | MENG, Raymond | antibody therapy for patients with | |
| | D.; . . . DOSING FOR | relapsed/refractory multiple myeloma: | |
| | TREATMENT WITH | differential mechanisms of action and | |
| | ANTI-TIGIT AND | recent clinical trial outcomes. | |
| | ANTI-CD20 OR ANTI- | 2022 Vial G, Lafargue . . . [Interest of | |
| | CD38 ANTIBODIES | daratumumab in refractory AL amyloidosis | |
| | 2021 | in a 96-year-old patient]. | |
| | US20210403592 | 2022 Shi Y, Li J, Zhang L Daratumumab for | |
| | Ahmadi, Tahamtan; . . . | the treatment of refractory idiopathic | |
| | Immune Modulation | multicentric Castleman disease: a case | |
| | and Treatment of | report. | |
| | Solid Tumors with | 2022 Palladini G, Mila . . . Advances in the | |
| | Antibodies that | treatment of light chain amyloidosis. | |
| | Specifically Bind | 2022 Singh A, Bazzi T, . . . Choroidal effusion: | |
| | CD38 | a rare and unusual complication of | |
| | 2021 | daratumumab. | |
| | US20210338766 | 2022 Aung F, Spencer J . . . Efficient | |
| | Klippel, Zandra; | neutralization of daratumumab in | |
| | METHODS OF | pretransfusion samples using a novel | |
| | TREATING MULTIPLE | recombinant monoclonal anti-idiotype | |
| | MYELOMA | antibody. | |
| | 2021 WO2021222524 | 2022 He J, Berringer H . . . Indirect | |
| | KLIPPEL, Zandra | Treatment Comparison of Daratumumab, | |
| | METHODS OF | Pomalidomide, and Dexamethasone | |
| | TREATING MULTIPLE | Versus Standard of Care in Patients with | |
| | MYELOMA WITH A | Difficult-to-Treat Relapsed/Refractory | |
| | TRIPLET THERAPY OF | Multiple Myeloma. | |
| | CARFILZOMIB, | 2022 Tokai T, Takashio . . . Assessing the | |
| | DEXAMETHASONE, | treatment effect of daratumumab by serial | |
| | AND AN ANTIBODY | measurements of cardiac biomarkers and | |
| | THAT SPECIFICALLY | imaging parameters in light-chain cardiac | |
| | RECOGNIZES CD38. | amyloidosis. | |
| | 2021 WO2021195362 | 2022 Jakubowiak AJ, Ku . . . Daratumumab | |
| | CAMPBELL, Mary, V . . . | Improves Depth of Response and | |
| | METHODS OF | Progression-free Survival in Transplant- | |
| | TREATING MULTIPLE | ineligible, High-risk, Newly Diagnosed | |
| | MYELOMA | Multiple Myeloma. | |
| | 2021 WO2021144457 | 2022 Fenton M, Shaw K, . . . Daratumumab | |
| | CLAUSEN, Jacob, D . . . | provides transient response of antibody | |
| | FORMULATIONS OF | mediated rejection post pediatric | |
| | CD38 ANTIBODIES | orthotopic heart transplantation. | |
| | AND USES THEREOF | 2022 Shah B, Gray J, A . . . Pharmacy | |
| | 2020 WO2021134045 | considerations: Use of anti-CD38 | |
| | BYRD, John C., HE . . . | monoclonal antibodies in relapsed and/or | |
| | METHODS AND | refractory multiple myeloma. | |
| | COMPOSITIONS FOR | 2022 Barbora V, Michae . . . CD38: A target | |
| | INHIBITION OF | in relapsed/refractory acute lymphoblastic | |
| | DIHYDROOROTATE | leukemia-Limitations in treatment and | |
| | DEHYDROGENASE IN | diagnostics. | |
| | COMBINATION WITH | 2022 Mele G, Cascavill . . . Daratumumab | |
| | AN ANTI-CD38 | plus bortezomib or daratumumab plus | |
| | THERAPEUTIC AGENT | lenalidomide as salvage therapy for | |
| | 2020 | patients with myeloma: initial follow-up of | |
| | US20210095042 | an Italian multicentre retrospective clinical | |
| | Jansson, Richard; . . . | experience by ‘Rete Ematologica Pugliese’. | |
| | Subcutaneous | 2022 Kawano Y, Hata H, . . . Daratumumab, | |
| | Formulations Of Anti- | lenalidomide and dexamethasone in newly | |
| | CD38 Antibodies And | diagnosed systemic light chain amyloidosis | |
| | Their Uses | patients associated with multiple | |
| | 2020 | myeloma. | |
| | US20210107991 | 2022 Dittus C, Miller . . . Daratumumab with | |
| | Jansson, Richard; . . . | ifosfamide, carboplatin and etoposide for | |
| | Subcutaneous | the treatment of relapsed plasmablastic | |
| | Formulations Of Anti- | lymphoma. | |
| | CD38 Antibodies And | 2022 Müller K, Vogiatz . . . Combining | |
| | Their Uses | daratumumab with CD47 blockade | |
| | 2020 WO2021089854 | prolongs survival in preclinical models of | |
| | O'DWYER, Michael, . . . | pediatric T-ALL. | |
| | TREATMENT OF | 2022 Moreno DF, Clapés . . . Real-World | |
| | MULTIPLE MYELOMA | Evidence of Daratumumab Monotherapy | |
| | 2020 WO2021092171 | in Relapsed/Refractory Multiple Myeloma | |
| | HUANG, Huang, | Patients and Efficacy on Soft-Tissue | |
| | RAV . . . DIAGNOSTIC | Plasmacytomas. | |
| | AND THERAPEUTIC | 2022 Matsue K, Sunami . . . Pomalidomide, | |
| | METHODS FOR | dexamethasone, and daratumumab in | |
| | TREATMENT OF | Japanese patients with relapsed or | |
| | HEMATOLOGIC | refractory multiple myeloma after | |
| | CANCERS | lenalidomide-based treatment. | |
| | 2020 | 2022 Blair HA Daratumumab: A Review in | |
| | US20220389103 | Newly Diagnosed Systemic Light Chain | |
| | HUANG, Huang; | Amyloidosis. | |
| | RAV . . . DIAGNOSTIC | 2022 Nooka AK, Kaufman . . . Daratumumab | |
| | AND THERAPEUTIC | plus | |
| | METHODS FOR | lenalidomide/bortezomib/dexamethasone | |
| | TREATMENT OF | in Black patients with transplant-eligible | |
| | HEMATOLOGIC | newly diagnosed multiple myeloma in | |
| | CANCERS | GRIFFIN. | |
| | 2020 | 2022 Gozzetti A, Ciofi . . . Anti CD38 | |
| | US20210061920 | monoclonal antibodies for multiple | |
| | Doshi, Parul; | myeloma treatment. | |
| | COMBINATION | 2022 Gao Y, Li L, Zhen . . . Monoclonal | |
| | THERAPIES WITH | antibody Daratumumab promotes | |
| | ANTI-CD38 | macrophage-mediated anti-myeloma | |
| | ANTIBODIES | phagocytic activity via engaging FC gamma | |
| | 2020 | receptor and activation of macrophages. | |
| | US20210047401 | 2022 Yu EY, Kolinsky M . . . Pembrolizumab | |
| | Doshi, Parul; Dan . . . | Plus Docetaxel and Prednisone in Patients | |
| | Anti-CD38 Antibodies | with Metastatic Castration-resistant | |
| | for Treatment of | Prostate Cancer: Long-term Results from | |
| | Acute Myeloid | the Phase 1b/2 KEYNOTE-365 Cohort B | |
| | Leukemia | Study. | |
| | 2020 | 2022 LeBlanc R, Mian H . . . Outcomes of | |
| | US20200407459 | daratumumab in the treatment of multiple | |
| | Chaulagain, Chakr . . . | myeloma: A retrospective cohort study | |
| | Anti-CD38 Antibodies | from the Canadian Myeloma Research | |
| | for Treatment of | Group Database. | |
| | Light Chain | 2022 Sanchorawala V, P . . . Health-related | |
| | Amyloidosis and | quality of life in patients with light chain | |
| | Other CD38-Positive | amyloidosis treated with bortezomib, | |
| | Hematological | cyclophosphamide, and | |
| | Malignancies | dexamethasone ± daratumumab: Results | |
| | 2020 | from the ANDROMEDA study. | |
| | US20200339701 | 2022 Atrash S, Thompso . . . Patient | |
| | Jansson, Richard; . . . | characteristics, treatment patterns, and | |
| | Subcutaneous | outcomes among black and white patients | |
| | Formulations Of Anti- | with multiple myeloma initiating | |
| | CD38 Antibodies And | daratumumab: A real-world chart review | |
| | Their Uses | study. | |
| | 2020 WO2020212914 | 2022 Davis JA, Youngbe . . . ‘Fast but not so | |
| | LIU, Xiangyang, S . . . | Furious': Short observation time after | |
| | COMBINATION | subcutaneous Daratumumab | |
| | THERAPIES | administration is both a safe and cost- | |
| | COMPRISING | effective strategy. | |
| | DARATUMUMAB, | 2022 Tiew HW, Sampath . . . Single-agent | |
| | BORTEZOMIB, | daratumumab for refractory POEMS | |
| | THALIDOMIDE AND | syndrome. | |
| | DEXAMETHASONE | 2022 Hughes DM, Hensha . . . Standard 30- | |
| | AND THEIR USES | minute Monitoring Time and Less Intensive | |
| | 2020 WO2020212912 | Pre-medications is Safe in Patients Treated | |
| | LIU, Xiangyang, S . . . | With Subcutaneous Daratumumab for | |
| | COMBINATION | Multiple Myeloma and Light Chain | |
| | THERAPIES | Amyloidosis. | |
| | COMPRISING | 2022 Benoit SW, Khande . . . A case of | |
| | DARATUMUMAB, | treatment-resistant membranous | |
| | BORTEZOMIB, | nephropathy associated with graft versus | |
| | THALIDOMIDE AND | host disease successfully treated with | |
| | DEXAMETHASONE | daratumumab. | |
| | AND THEIR USES | 2022 Kassem S, Diallo . . . SAR442085, a | |
| | 2020 WO2020212911 | novel anti-CD38 antibody with enhanced | |
| | LIU, Xiangyang, S . . . | antitumor activity against multiple | |
| | COMBINATION | myeloma. | |
| | THERAPIES | 2022 Bahlis NJ, Siegel . . . Pomalidomide, | |
| | COMPRISING | dexamethasone, and daratumumab | |
| | DARATUMUMAB, | immediately after lenalidomide-based | |
| | BORTEZOMIB, | treatment in patients with multiple | |
| | THALIDOMIDE AND | myeloma: updated efficacy, safety, and | |
| | DEXAMETHASONE | health-related quality of life results from | |
| | AND THEIR USES | the phase 2 MM-014 trial. | |
| | 2020 | 2022 Scheibe F, Ostend . . . Daratumumab | |
| | US20200231697 | for treatment-refractory antibody- | |
| | Jansson, Richard; . . . | mediated diseases in neurology. | |
| | Subcutaneous | 2022 Baloda V, Shurin . . . Pilot Verification | |
| | Formulations Of Anti- | of a Novel Approach to Remove | |
| | CD38 Antibodies And | Electrophoretic Interference of the | |
| | Their Uses | Therapeutic Monoclonal Antibody | |
| | 2020 U.S. Pat. No. 11,566,079 | Daratumumab. | |
| | Jansson, Richard; . . . | 2022 Dimopoulos MA, Ri . . . Treatment | |
| | Subcutaneous | Options for Patients With Heavily | |
| | formulations of anti- | Pretreated Relapsed and Refractory | |
| | CD38 antibodies and | Multiple Myeloma. | |
| | their uses | 2022 Coffman K, Carste . . . Daratumumab | |
| | 2020 | infusion reaction rates pre- and post- | |
| | US20200308296 | addition of montelukast to pre- | |
| | Bandekar, Rajesh; . . . | medications. | |
| | Clinically Proven | 2021 Arnall JR, Maples . . . Daratumumab for | |
| | Subcutaneous | the Treatment of Multiple Myeloma: A | |
| | Pharmaceutical | Review of Clinical Applicability and | |
| | Compositions | Operational Considerations. | |
| | Comprising Anti- | 2021 Maouche N, Sriniv . . . Daratumumab | |
| | CD38 Antibodies and | Monotherapy for Heavily Pre-treated and | |
| | Their Uses in | Refractory Myeloma: Results from a UK | |
| | Combination with | Multicentre Real World Cohort. | |
| | Pomalidomide and | 2021 Wong XY, Chng WJ, . . . Cost- | |
| | Dexamethasone | effectiveness of daratumumab in | |
| | 2020 | combination with lenalidomide and | |
| | US20200308297 | dexamethasone for relapsed and/or | |
| | Bandekar, Rajesh; . . . | refractory multiple myeloma. | |
| | Clinically Proven | 2021 Costa LJ, Chhabra . . . Daratumumab, | |
| | Subcutaneous | Carfilzomib, Lenalidomide, and | |
| | Pharmaceutical | Dexamethasone With Minimal Residual | |
| | Compositions | Disease Response-Adapted Therapy in | |
| | Comprising Anti- | Newly Diagnosed Multiple Myeloma. | |
| | CD38 Antibodies and | 2021 Usmani SZ, Quach . . . Carfilzomib, | |
| | Their Uses | dexamethasone, and daratumumab versus | |
| | 2020 | carfilzomib and dexamethasone for | |
| | US20200308284 | patients with relapsed or refractory | |
| | Bandekar, Rajesh; . . . | multiple myeloma (CANDOR): updated | |
| | Clinically Proven | outcomes from a randomised, multicentre, | |
| | Subcutaneous | open-label, phase 3 study. | |
| | Pharmaceutical | 2021 Zhou Y, Chen L, J . . . 2- | |
| | Compositions | Mercaptoethanol (2-ME)-based IATs or | |
| | Comprising Anti- | Polybrene method mitigates the | |
| | CD38 Antibodies and | interference of daratumumab on blood | |
| | Their Uses in | compatibility tests. | |
| | Combination with | 2021 Chim CS, Kumar S, . . . 3-weekly | |
| | Lenalidomide and | daratumumab- | |
| | Dexamethasone | lenalidomide/pomalidomide- | |
| | 2020 WO2020194242 | dexamethasone is highly effective in | |
| | BANDEKAR, Rajesh, . . . | relapsed and refractory multiple myeloma. | |
| | CLINICALLY PROVEN | 2021 Narsipur N, Bulla . . . Cost-effectiveness | |
| | SUBCUTANEOUS | of adding daratumumab or bortezomib to | |
| | PHARMACEUTICAL | lenalidomide plus dexamethasone for | |
| | COMPOSITIONS | newly diagnosed multiple myeloma. | |
| | COMPRISING ANTI- | 2021 Gil-Sierra MD, Br . . . Daratumumab- | |
| | CD38 ANTIBODIES | based therapies in transplant-ineligible | |
| | AND THEIR USES IN | patients with untreated multiple myeloma | |
| | COMBINATION WITH | and hepatic dysfunction: A systematic | |
| | POMALIDOMIDE AND | review of subgroup analyses. | |
| | DEXAMETHASONE | 2021 Luo MM, Zhu PP, N . . . Population | |
| | 2020 WO2020194244 | Pharmacokinetics and Exposure-Response | |
| | BANDEKAR, Rajesh, . . . | Modeling of Daratumumab Subcutaneous | |
| | CLINICALLY PROVEN | Administration in Patients With Light-Chain | |
| | SUBCUTANEOUS | Amyloidosis. | |
| | PHARMACEUTICAL | 2021 Theodorakakou F, . . . Daratumumab | |
| | COMPOSITIONS | plus CyBorD for patients with newly | |
| | COMPRISING ANTI- | diagnosed light chain (AL) amyloidosis. | |
| | CD38 ANTIBODIES | 2021 Atrash S, Thompso . . . Treatment | |
| | AND THEIR USES IN | patterns and effectiveness of patients with | |
| | COMBINATION WITH | multiple myeloma initiating Daratumumab | |
| | BORTEZOMIB AND | across different lines of therapy: a real- | |
| | DEXAMETHASONE | world chart review study. | |
| | 2020 WO2020194243 | 2021 Farber M, Chen Y, . . . Targeting CD38 | |
| | BANDEKAR, Rajesh, . . . | in acute myeloid leukemia interferes with | |
| | CLINICALLY PROVEN | leukemia trafficking and induces | |
| | SUBCUTANEOUS | phagocytosis. | |
| | PHARMACEUTICAL | 2021 Duray E, Lejeune . . . A non- | |
| | COMPOSITIONS | internalised CD38-binding radiolabelled | |
| | COMPRISING ANTI- | single-domain antibody fragment to | |
| | CD38 ANTIBODIES | monitor and treat multiple myeloma. | |
| | AND THEIR USES IN | 2021 Liu Y, Huang XH, . . . [Daratumumab | |
| | COMBINATION WITH | for the treatment of primary systemic | |
| | LENALIDOMIDE AND | amyloidosis: a multicenter retrospective | |
| | DEXAMETHASONE | analysis]. | |
| | 2020 WO2020194245 | 2021 Ehsan H, Rafae A, . . . Efficacy and | |
| | BANDEKAR, Rajesh, . . . | Safety of Daratumumab-based Regimens | |
| | CLINICALLY PROVEN | in Pretreated Light Chain (AL) Amyloidosis: | |
| | SUBCUTANEOUS | A Systematic Review. | |
| | PHARMACEUTICAL | 2021 Kang L, Li C, Yan . . . 64Cu-labeled | |
| | COMPOSITIONS | daratumumab F(ab′)2 fragment enables | |
| | COMPRISING ANTI- | early visualization of CD38-positive | |
| | CD38 ANTIBODIES | lymphoma. | |
| | AND THEIR USES IN | 2021 Tam AH, Jung Y, Y . . . Evaluation of | |
| | COMBINATION WITH | subcutaneous daratumumab injections in | |
| | BORTEZOMIB, | the ambulatory care setting. | |
| | MELPHALAN AND | 2021 Broijl A, de Jong . . . VS38c and CD38- | |
| | PREDNISONE | Multiepitope Antibodies Provide Highly | |
| | 2020 WO2020194241 | Comparable Minimal Residual Disease | |
| | BANDEKAR, Rajesh, . . . | Data in Patients With Multiple Myeloma. | |
| | CLINICALLY PROVEN | 2021 Tauscher C, Molde . . . Antibody | |
| | SUBCUTANEOUS | incidence and red blood cell transfusions in | |
| | PHARMACEUTICAL | patients on daratumumab. | |
| | COMPOSITIONS | 2021 Van den Berg J, K . . . Daratumumab for | |
| | COMPRISING ANTI- | immune thrombotic thrombocytopenia | |
| | CD38 ANTIBODIES | purpura. | |
| | AND THEIR USES | 2021 Panaampon J, Kari . . . Efficacy and | |
| | 2020 | mechanism of the anti-CD38 monoclonal | |
| | US20200316197 | antibody Daratumumab against primary | |
| | Bandekar, Rajesh; . . . | effusion lymphoma. | |
| | Clinically Proven | 2021 Ibeh N, Baine I, . . . Use of an in-house | |
| | Subcutaneous | trypsin-based method to resolve the | |
| | Pharmaceutical | interference of daratumumab. | |
| | Compositions | 2021 Rybinski B, Kocog . . . Hepatic AL | |
| | Comprising Anti- | Amyloidosis without Significant Light Chain | |
| | CD38 Antibodies and | Elevation in a Patient Treated with CyBorD | |
| | Their Uses in | Plus Daratumumab. | |
| | Combination with | 2021 Bullock T, Foster . . . Alloimmunisation | |
| | Bortezomib and | rate of patients on Daratumumab: A | |
| | Dexamethasone | retrospective cohort study of patients in | |
| | 2020 | England. | |
| | US20200330593 | 2021 Noori S, Verkleij . . . Monitoring the M- | |
| | Bandekar, Rajesh; . . . | protein of multiple myeloma patients | |
| | Clinically Proven | treated with a combination of monoclonal | |
| | Subcutaneous | antibodies: the laboratory solution to | |
| | Pharmaceutical | eliminate interference. | |
| | Compositions | 2021 Epperly R, Santia . . . Targeting plasma | |
| | Comprising Anti- | cells with daratumumab aids in the | |
| | CD38 Antibodies and | treatment of post-transplant autoimmune- | |
| | Their Uses in | like hepatitis. | |
| | Combination with | 2021 Bigley AB, Spade . . . FcεRlγ-negative | |
| | Bortezomib, | NK cells persist in vivo and enhance | |
| | Mephalan and | efficacy of therapeutic monoclonal | |
| | Prednisone | antibodies in multiple myeloma. | |
| | 2020 WO2020183147 | 2021 Yu N, Zhang Y, Li . . . Daratumumab | |
| | PEDDAREDDIGARI, | Immunopolymersome-Enabled Safe and | |
| | V . . . | CD38-Targeted Chemotherapy and | |
| | IMMUNOTHERAPY | Depletion of Multiple Myeloma. | |
| | COMBINED WITH AN | 2021 Yamazaki T, Joshi . . . Successful | |
| | ANTI-CD38 | treatment by glecaprevir/pibrentasvir | |
| | ANTIBODY | followed by hepatoprotective therapy of | |
| | 2020 WO2020185672 | acute chronic hepatitis exacerbation | |
| | JORDAN, Stanley C . . . | caused by daratumumab-based regimen | |
| | ANTI-CD38 AGENTS | for multiple myeloma: Case report and | |
| | FOR | review of the literature. | |
| | DESENSITIZATION | 2021 Xing L, Wang S, L . . . BCMA-specific | |
| | AND TREATMENT OF | ADC MEDI2228 and Daratumumab induce | |
| | ANTIBODY- | synergistic myeloma cytotoxicity via IFN- | |
| | MEDIATED | driven immune responses and enhanced | |
| | REJECTION OF | CD38 expression. | |
| | ORGAN | 2021 Kirchhoff DC, Mur . . . Use of a | |
| | TRANSPLANTS | Daratumumab-Specific Immunofixation | |
| | 2020 | Assay to Assess Possible Immunotherapy | |
| | US20220135695 | Interference at a Major Cancer Center: Our | |
| | JORDAN, Stanley C . . . | Experience and Recommendations. | |
| | ANTI-CD38 AGENTS | 2021 Kitadate A, Terao . . . Multiple | |
| | FOR | myeloma with t(11; 14)-associated | |
| | DESENSITIZATION | immature phenotype has lower CD38 | |
| | AND TREATMENT OF | expression and higher BCL2 dependence. | |
| | ANTIBODY- | 2021 Kleinot W, Aguile . . . Daratumumab | |
| | MEDIATED | Interference in Flow Cytometry Producing | |
| | REJECTION OF | a False Kappa Light Chain Restriction in | |
| | ORGAN | Plasma Cells. | |
| | TRANSPLANTS | 2021 Kastritis E, Pall . . . Daratumumab- | |
| | 2020 WO2020176748 | Based Treatment for Immunoglobulin | |
| | MENG, Raymond, D. | Light-Chain Amyloidosis. | |
| | DOSING FOR | 2021 Mustafa N, Nee AH . . . Determinants | |
| | TREATMENT WITH | of response to daratumumab in Epstein- | |
| | ANTI-TIGIT AND | Barr virus-positive natural killer and T-cell | |
| | ANTI-CD20 OR ANTI- | lymphoma. | |
| | CD38 ANTIBODIES | 2021 Shah N, Perales M . . . Phase I study | |
| | 2020 | protocol: NKTR-255 as monotherapy or | |
| | US20200268847 Qi, | combined with daratumumab or rituximab | |
| | Ming; Methods of | in hematologic malignancies. | |
| | Treating Newly | 2021 Liu L, Fiala M, G . . . A single center | |
| | Diagnosed Multiple | retrospective study of daratumumab, | |
| | Myeloma with a | pomalidomide, and dexamethasone as | |
| | Combination of An | 2nd-line therapy in multiple myeloma. | |
| | Antibody that | 2021 Stikvoort A, van . . . CD38-specific | |
| | Specifically Binds | Chimeric Antigen Receptor Expressing | |
| | CD38, Lenalidomide | Natural Killer KHYG-1 Cells: A Proof of | |
| | and Dexamethasone | Concept for an “Off the Shelf” Therapy for | |
| | 2020 WO2020170211 | Multiple Myeloma. | |
| | QI, Ming METHODS | 2021 Regidor B, Goldwa . . . Low dose | |
| | OF TREATING NEWLY | venetoclax in combination with | |
| | DIAGNOSED | bortezomib, daratumumab, and | |
| | MULTIPLE MYELOMA | dexamethasone for the treatment of | |
| | WITH A | relapsed/refractory multiple myeloma | |
| | COMBINATION OF | patients-a single-center retrospective | |
| | AN ANTIBODY THAT | study. | |
| | SPECIFICALLY BINDS | 2021 Thangaraj JL, Ahn . . . Expanded natural | |
| | CD38, | killer cells augment the antimyeloma | |
| | LENALIDOMIDE AND | effect of daratumumab, bortezomib, and | |
| | DEXAMETHASONE | dexamethasone in a mouse model. | |
| | 2020 | 2021 Davies F, Rifkin . . . Real-world | |
| | US20200283542 DE | comparative effectiveness of triplets | |
| | WEERS, Michel; . . . | containing bortezomib (B), carfilzomib (C), | |
| | ANTIBODIES AGAINST | daratumumab (D), or ixazomib (I) in | |
| | CD38 FOR | relapsed/refractory multiple myeloma | |
| | TREATMENT OF | (RRMM) in the US. | |
| | MULTIPLE MYELOMA | 2021 Phou S, Costello . . . Optimizing | |
| | 2020 | transfusion management of multiple | |
| | US20200165352 | myeloma patients receiving daratumumab- | |
| | GOEIJ, Bart De; A . . . | based regimens. | |
| | VARIANTS OF CD38 | 2021 Piggin A, Prince HM An evaluation of | |
| | ANTIBODY AND USES | isatuximab, pomalidomide and | |
| | THEREOF | dexamethasone for adult patients with | |
| | 2020 | relapsed and refractory multiple myeloma. | |
| | US20200239589 | 2021 Kimmich CR, Terze . . . Daratumumab, | |
| | AUDAT, Heloise; B . . . | lenalidomide, and dexamethasone in | |
| | METHODS OF | systemic light-chain amyloidosis: High | |
| | TREATING MULTIPLE | efficacy, relevant toxicity and main adverse | |
| | MYELOMA | effect of gain 1q21. | |
| | 2020 | 2021 Driouk L, Schmitt . . . Daratumumab | |
| | US20200223936 | therapy for post-HSCT immune-mediated | |
| | Doshi, Parul; Anti- | cytopenia: experiences from two pediatric | |
| | CD38 Antibodies for | cases and review of literature. | |
| | Treatment of Acute | 2021 van der Horst HJ, . . . Potent preclinical | |
| | Lymphoblastic | activity of HexaBody-DR5/DR5 in relapsed | |
| | Leukemia | and/or refractory multiple myeloma. | |
| | 2019 | 2021 Verkleij CPM, Bro . . . Preclinical | |
| | US20200148782 | activity and determinants of response of | |
| | Larmore, Nicole; . . . | the GPRC5D × CD3 bispecific antibody | |
| | Control of Trace | talquetamab in multiple myeloma. | |
| | Metals During | 2021 Lu J, Fu W, Li W, . . . Daratumumab, | |
| | Production of Anti- | Bortezomib, and Dexamethasone Versus | |
| | CD38 Antibodies | Bortezomib and Dexamethasone in | |
| | 2019 WO2020100073 | Chinese Patients with Relapsed or | |
| | LARMORE, Nicole, . . . | Refractory Multiple Myeloma: Phase 3 | |
| | CONTROL OF TRACE | LEPUS (MMY3009) Study. | |
| | METALS DURING | 2021 Henig I, Yehudai - . . . Pure Red Cell | |
| | PRODUCTION OF | Aplasia following ABO-Mismatched | |
| | ANTI-CD38 | Allogeneic Hematopoietic Stem Cell | |
| | ANTIBODIES | Transplantation: Resolution with | |
| | 2019 | Daratumumab Treatment. | |
| | US20200121588 | 2021 Patel A, Clark S, . . . Retrospective | |
| | Campbell, Kim Ann . . . | review of accelerated daratumumab | |
| | Method of Providing | administration. | |
| | Subcutaneous | 2021 Delforge M, Vlaye . . . | |
| | Administration of | Immunomodulators in newly diagnosed | |
| | Anti-CD38 Antibodies | multiple myeloma: current and future | |
| | 2019 WO2020081881 | concepts. | |
| | CAMPBELL, Kim | 2021 Rosé M, Bourahla . . . Assessment of | |
| | Ann . . . METHOD OF | Healthcare Professionals' Knowledge and | |
| | PROVIDING | Understanding of the Risk of Blood Typing | |
| | SUBCUTANEOUS | Interference with Daratumumab: A Survey | |
| | ADMINISTRATION OF | of 12 European Countries. | |
| | ANTI-CD38 | 2021 Zand L, Rajkumar . . . Safety and | |
| | ANTIBODIES | Efficacy of Daratumumab in Patients with | |
| | 2019 WO2020072334 | Proliferative GN with Monoclonal | |
| | AMATANGELO, | Immunoglobulin Deposits. | |
| | Micha . . . | 2021 Jeryczynski G, An . . . First-line | |
| | COMBINATION | daratumumab shows high efficacy and | |
| | THERAPY FOR THE | tolerability even in advanced AL | |
| | TREATMENT OF | amyloidosis: the real-world experience. | |
| | CANCER | 2021 Aguilera Agudo C, . . . Daratumumab | |
| | 2019 | for Antibody-mediated Rejection in Heart | |
| | US20200114000 VAN | Transplant-A Novel Therapy: Successful | |
| | DE WINKEL, Ja . . . | Treatment of Antibody-mediated | |
| | COMBINATION | Rejection. | |
| | TREATMENT OF | 2021 Cook G, Corso A, . . . Daratumumab | |
| | CD38-EXPRESSING | Monotherapy for Relapsed or Refractory | |
| | TUMORS | Multiple Myeloma: Results of an Early | |
| | 2019 | Access Treatment Protocol in Europe and | |
| | US20200199229 VAN | Russia. | |
| | DEN BRINK, Ed . . . | 2021 Richter J, Anupin . . . Real-world | |
| | HUMANIZED OR | treatment patterns in relapsed/refractory | |
| | CHIMERIC CD3 | multiple myeloma: Clinical and economic | |
| | ANTIBODIES | outcomes in patients treated with | |
| | 2019 | pomalidomide or daratumumab. | |
| | US20200017600 | 2021 Iida S, Ishikawa . . . Subcutaneous | |
| | GOEIJ, Bart De; A . . . | daratumumab in Asian patients with | |
| | VARIANTS OF CD38 | heavily pretreated multiple myeloma: | |
| | ANTIBODY AND USES | subgroup analyses of the noninferiority, | |
| | THEREOF | phase 3 COLUMBA study. | |
| | 2019 WO2020012038 | 2021 Cho H, Kim KH, Le . . . Adaptive natural | |
| | DE GOEIJ, Bart, E, . . . | killer cells facilitate effector functions of | |
| | TROGOCYTOSIS- | daratumumab in multiple myeloma. | |
| | MEDIATED THERAPY | 2021 Wang C, Chen Y, H . . . ImmunoPET | |
| | USING CD38 | imaging of multiple myeloma with | |
| | ANTIBODIES | [68Ga]Ga-NOTA-Nb1053. | |
| | 2019 WO2020012036 | 2021 Carrón-Herrero A, . . . Successful | |
| | DE GOEIJ, Bart, E, . . . | Desensitization to Daratumumab after | |
| | VARIANTS OF CD38 | Severe Life-Threatening Reaction in A | |
| | ANTIBODY AND USES | Patient with Refractory Multiple Myeloma. | |
| | THEREOF | 2021 Yates B, Molloy E . . . Daratumumab for | |
| | 2019 | delayed RBC engraftment following major | |
| | US20200002433 | ABO mismatched haploidentical bone | |
| | Jansson, Richard; . . . | marrow transplantation. | |
| | Subcutaneous | 2021 Eveillard M, Kord . . . Using MALDI-TOF | |
| | Formulations Of Anti- | mass spectrometry in peripheral blood for | |
| | CD38 Antibodies And | the follow up of newly diagnosed multiple | |
| | Their Uses | myeloma patients treated with | |
| | 2019 | daratumumab-based combination therapy. | |
| | US20190330363 | 2021 Vozella F, Sinisc . . . Daratumumab in | |
| | Jansson, Richard; . . . | multiple myeloma: experience of the | |
| | Subcutaneous | multiple myeloma GIMEMA Lazio group. | |
| | Formulations Of Anti- | 2021 Doberer K, Kläger . . . CD38 Antibody | |
| | CD38 Antibodies And | Daratumumab for the Treatment of | |
| | Their Uses | Chronic Active Antibody-mediated Kidney | |
| | 2019 U.S. Pat. No. 10,781,261 | Allograft Rejection. | |
| | Jansson, Richard . . . | 2021 Ueno T, Sugio Y, . . . Successful upfront | |
| | Subcutaneous | cord blood transplantation for plasma cell | |
| | formulations of anti- | leukemia in the first complete response | |
| | CD38 antibodies and | after daratumumab therapy. | |
| | their uses | 2021 Chari A, Munder M . . . Evaluation of | |
| | 2019 | Cardiac Repolarization in the Randomized | |
| | US20190298827 Xie, | Phase 2 Study of Intermediate- or High- | |
| | Hong; Cakana . . . | Risk Smoldering Multiple Myeloma | |
| | Methods of Treating | Patients Treated with Daratumumab | |
| | Multiple Myeloma | Monotherapy. | |
| | 2019 WO2019173902 | 2021 Parrondo RD, Mous . . . Efficacy of | |
| | LIN, Gloria Hoi Y . . . | Daratumumab-Based Regimens for the | |
| | CD47 BLOCKADE | Treatment of Plasma Cell Leukemia. | |
| | THERAPY WITH CD38 | 2020 Lee HT, Kim Y, Pa . . . Crystal structure | |
| | ANTIBODY | of CD38 in complex with daratumumab, a | |
| | 2019 | first-in-class anti-CD38 antibody drug for | |
| | US20210040224 LIN, | treating multiple myeloma. | |
| | Gloria Hoi Y . . . CD47 | 2020 Touzeau C, Antier . . . Carfilzomib in | |
| | BLOCKADE THERAPY | combination with daratumumab in the | |
| | WITH CD38 | management of relapsed multiple | |
| | ANTIBODY | myeloma. | |
| | 2018 WO2019094931 | 2020 Korst CLBM, van d . . . Should all newly | |
| | SETH, Sandesh, TH . . . | diagnosed MM patients receive CD38 | |
| | COMBINATION | antibody-based treatment? | |
| | THERAPY FOR | 2020 Roussel M, Moreau . . . Bortezomib, | |
| | TREATMENT OF A | thalidomide, and dexamethasone with or | |
| | HEMATOLOGICAL | without daratumumab for transplantation- | |
| | DISEASE | eligible patients with newly diagnosed | |
| | 2018 | multiple myeloma (CASSIOPEIA): health- | |
| | US20200283539 | related quality of life outcomes of a | |
| | SETH, Sandesh; TH . . . | randomised, open-label, phase 3 trial. | |
| | COMBINATION | 2020 Scheid C, Blau IW . . . Changes in | |
| | THERAPY FOR | treatment landscape of relapsed or | |
| | TREATMENT OF A | refractory multiple myeloma and their | |
| | HEMATOLOGICAL | association with mortality: Insights from | |
| | DISEASE | German claims database. | |
| | 2018 | 2020 Naeimi Kararoudi . . . CD38 deletion of | |
| | US20190144557 | human primary NK cells eliminates | |
| | Ahmadi, Tahamtan; . . . | daratumumab-induced fratricide and | |
| | Immune Modulation | boosts their effector activity. | |
| | and Treatment of | 2020 Ogiya D, Liu J, O . . . The JAK-STAT | |
| | Solid Tumors with | pathway regulates CD38 on myeloma cells | |
| | Antibodies that | in the bone marrow microenvironment: | |
| | Specifically Bind | therapeutic implications. | |
| | CD38 | 2020 Minnix M, Adhikar . . . Comparison of | |
| | 2018 U.S. Pat. No. 11,021,543 | CD38 targeted alpha- vs beta-radionuclide | |
| | Ahmadi, Tahamtan . . . | therapy of disseminated multiple myeloma | |
| | Immune modulation | in an animal model. | |
| | and treatment of | 2020 Dossier C, Prim B . . . A global antiB cell | |
| | solid tumors with | strategy combining obinutuzumab and | |
| | antibodies that | daratumumab in severe pediatric | |
| | specifically bind CD38 | nephrotic syndrome. | |
| | 2018 WO2018183821 | 2020 Krishnan A, Adhik . . . Identifying | |
| | SHRESTHA, Niraj, . . . | CD38+ cells in patients with multiple | |
| | ALT-803 IN | myeloma: first-in-human imaging using | |
| | COMBINATION WITH | copper-64-labeled daratumumab. | |
| | ANTI-CD38 | 2020 Godara A, Palladi . . . Monoclonal | |
| | ANTIBODY FOR | Antibody Therapies in Systemic Light-Chain | |
| | CANCER THERAPIES | Amyloidosis. | |
| | 2018 | 2020 Ostendorf L, Burn . . . Targeting CD38 | |
| | US20190048055 | with Daratumumab in Refractory Systemic | |
| | Shrestha, Niraj; . . . | Lupus Erythematosus. | |
| | ALT-803 IN | 2020 Lamb YN Daratumumab: A Review in | |
| | COMBINATION WITH | Combination Therapy for Transplant- | |
| | ANTI-CD38 | Eligible Newly Diagnosed Multiple | |
| | ANTIBODY FOR | Myeloma. | |
| | CANCER THERAPIES | 2020 Durie BGM, Kumar . . . Daratumumab- | |
| | 2018 | lenalidomide-dexamethasone vs standard- | |
| | US20180298106 DE | of-care regimens: Efficacy in transplant- | |
| | WEERS, Michel; . . . | ineligible untreated myeloma. | |
| | ANTIBODIES AGAINST | 2020 Lombardi J, Bouli . . . Safety of ninety- | |
| | HUMAN CD38 | minute daratumumab infusion. | |
| | 2018 U.S. Pat. No. 11,230,604 De | 2020 Lidický O, Klener . . . Overcoming | |
| | Weers, Michel . . . | resistance to rituximab in relapsed non- | |
| | Antibodies against | Hodgkin lymphomas by antibody-polymer | |
| | human CD38 | drug conjugates actively targeted by anti- | |
| | 2018 WO2018136961 | CD38 daratumumab. | |
| | MCKEOWN, | 2020 Kastritis E, Theo . . . Daratumumab- | |
| | Michael, . . . METHODS | based therapy for patients with | |
| | OF TREATING | monoclonal gammopathy of renal | |
| | PATIENTS WITH A | significance. | |
| | RETINOIC ACID | 2020 Jiao Y, Yi M, Xu . . . CD38: Targeted | |
| | RECEPTOR-α | therapy in multiple myeloma and | |
| | AGONIST AND AN | therapeutic potential for solid cancers. | |
| | ANTI-CD38 | 2020 Susek KH, Gran C, . . . Outcome of | |
| | ANTIBODY | COVID-19 in multiple myeloma patients in | |
| | 2017 | relation to treatment. | |
| | US20180117150 | 2020 Chari A, Rodrigue . . . Subcutaneous | |
| | O'Dwyer, Michael; . . . | daratumumab plus standard treatment | |
| | Combination | regimens in patients with multiple | |
| | Therapies for CD38- | myeloma across lines of therapy | |
| | Positive | (PLEIADES): an open-label Phase II study. | |
| | Hematological | 2020 Mizuno S, Kitayam . . . Successful | |
| | Malignances with | management of hemodialysis-dependent | |
| | ANTI-CD38 | refractory myeloma with modified | |
| | Antibodies and | daratumumab, bortezomib and | |
| | Cyclophosphamide | dexamethasone regimen. | |
| | 2017 WO2018015498 | 2020 Roccatello D, Fen . . . Towards a novel | |
| | BENJAMIN, Susan, . . . | target therapy for renal diseases related to | |
| | COMBINATIONS OF | plasma cell dyscrasias: The example of AL | |
| | INECALCITOL WITH | amyloidosis. | |
| | AN ANTI-CD38 | 2020 Jeyaraman P, Bora . . . Daratumumab | |
| | AGENT AND THEIR | for pure red cell aplasia post ABO | |
| | USES FOR TREATING | incompatible allogeneic hematopoietic | |
| | CANCER | stem cell transplant for aplastic anemia. | |
| | 2017 | 2020 Edwards RG, Vande . . . Bilateral | |
| | US20170320961 | Secondary Angle Closure During | |
| | Doshi, Parul; Anti- | Daratumumab Infusion: A Case Report and | |
| | CD38 Antibodies for | Review of the Literature. | |
| | Treatment of Acute | 2020 Casneuf T, Adams . . . Deep immune | |
| | Lymphoblastic | profiling of patients treated with | |
| | Leukemia | lenalidomide and dexamethasone with or | |
| | 2017 U.S. Pat. No. 10,556,961 | without daratumumab. | |
| | Doshi, Parul (Che . . . | 2020 Cohen OC, Broderm . . . Rapid response | |
| | Anti-CD38 antibodies | to single agent daratumumab is associated | |
| | for treatment of | with improved progression-free survival in | |
| | acute lymphoblastic | relapsed/refractory AL amyloidosis. | |
| | leukemia | 2020 Kikuchi T, Kusumo . . . Hepatitis B virus | |
| | 2017 | reactivation in a myeloma patient with | |
| | US20170174780 | resolved infection who received | |
| | Doshi, Parul; | daratumumab-containing salvage | |
| | Combination | chemotherapy. | |
| | Therapies with Anti- | 2020 García-Guerrero E . . . Upregulation of | |
| | CD38 Antibodies | CD38 expression on multiple myeloma | |
| | 2017 U.S. Pat. No. 10,800,851 | cells by novel HDAC6 inhibitors is a class | |
| | Doshi, Parul (Che . . . | effect and augments the efficacy of | |
| | Combination | daratumumab. | |
| | therapies with anti- | 2020 Franssen LE, Steg . . . Resistance | |
| | CD38 antibodies | Mechanisms Towards CD38-Directed | |
| | 2016 | Antibody Therapy in Multiple Myeloma. | |
| | US20170107295 | 2020 Viola D, Dona A, . . . Daratumumab | |
| | Lokhorst, Henk M. . . . | induces mechanisms of immune activation | |
| | Combination | through CD38+ NK cell targeting. | |
| | Therapies with Anti- | 2020 Vakili H, Koorse . . . Complete | |
| | CD38 Antibodies | Depletion of Daratumumab Interference in | |
| | 2016 | Serum Samples from Plasma Cell Myeloma | |
| | US20170121417 | Patients Improves the Detection of | |
| | Jansson, Richard; . . . | Endogenous M-Proteins in a Preliminary | |
| | Subcutaneous | Study. | |
| | Formulations of Anti- | 2020 Ulaner GA, Sobol . . . CD38-targeted | |
| | CD38 Antibodies and | Immuno-PET of Multiple Myeloma: From | |
| | Their Uses | Xenograft Models to First-in-Human | |
| | 2016 | Imaging. | |
| | US20170044265 | 2020 Moore DC, Arnall . . . Dialysis | |
| | Ahmadi, Tahamtan; . . . | Independence Following Combination | |
| | Immune Modulation | Daratumumab, Thalidomide, Bortezomib, | |
| | and Treatment of | Cyclophosphamide, and Dexamethasone in | |
| | Solid Tumors with | Multiple Myeloma With Severe Renal | |
| | Antibodies that | Failure. | |
| | Specifically Bind | 2020 Palladini G, Kast . . . Daratumumab Plus | |
| | CD38 | CyBorD for Patients With Newly Diagnosed | |
| | 2016 | AL Amyloidosis: Safety Run-in Results of | |
| | US20170121414 | ANDROMEDA. | |
| | Jansson, Richard; . . . | 2020 Jain A, Ramasamy K Evolving Role of | |
| | Subcutaneous | Daratumumab: From Backbencher to | |
| | Formulations of Anti- | Frontline Agent. | |
| | CD38 Antibodies and | 2020 Hu Y, Liu H, Fang . . . Targeting of CD38 | |
| | Their Uses | by the Tumor Suppressor miR-26a Serves | |
| | 2016 WO2017079150 | as a Novel Potential Therapeutic Agent in | |
| | JANSSON, Richard, . . . | Multiple Myeloma. | |
| | SUBCUTANEOUS | 2020 Storti P, Vescovi . . . CD14+ CD16+ | |
| | FORMULATIONS OF | monocytes are involved in daratumumab- | |
| | ANTI-CD38 | mediated myeloma cells killing and in anti- | |
| | ANTIBODIES AND | CD47 therapeutic strategy. | |
| | THEIR USES | 2020 Gil-Sierra MD, Gi . . . Network meta- | |
| | 2016 U.S. Pat. No. 10,385,135 | analysis of first-line treatments in | |
| | Jansson, Richard . . . | transplant-ineligible multiple myeloma | |
| | Subcutaneous | patients. | |
| | formulations of anti- | 2020 Roussel M, Merlin . . . A prospective | |
| | CD38 antibodies and | phase II of daratumumab in previously | |
| | their uses | treated systemic light chain amyloidosis | |
| | 2016 | (AL) patients. | |
| | US20160376373 | 2020 Xu XS, Moreau P, . . . Split First Dose | |
| | Ahmadi, Tahamtan; . . . | Administration of Intravenous | |
| | Immune Modulation | Daratumumab for the Treatment of | |
| | and Treatment of | Multiple Myeloma (MM): Clinical and | |
| | Solid Tumors with | Population Pharmacokinetic Analyses. | |
| | Antibodies that | 2020 Chung A, Kaufman . . . Organ | |
| | Specifically Bind | responses with daratumumab therapy in | |
| | CD38 | previously treated AL amyloidosis. | |
| | 2016 WO2016210223 | 2020 Stocker N, Gaugle . . . Daratumumab | |
| | TAHAMTAN, | prevents programmed death ligand-1 | |
| | Ahmadi, . . . IMMUNE | expression on antigen-presenting cells in | |
| | MODULATION AND | de novo multiple myeloma. | |
| | TREATMENT OF | 2020 Nikolaenko L, Chh . . . Graft-Versus- | |
| | SOLID TUMORS WITH | Host Disease in Multiple Myeloma Patients | |
| | ANTIBODIES THAT | Treated With Daratumumab After | |
| | SPECIFICALLY BIND | Allogeneic Transplantation. | |
| | CD38 | 2020 Sanchorawala V, S . . . Safety, | |
| | 2016 | Tolerability, and Response Rates of | |
| | US20160367663 | Daratumumab in Relapsed AL Amyloidosis: | |
| | Doshi, Parul; Lok . . . | Results of a Phase II Study. | |
| | Combination | 2020 Frerichs KA, Broe . . . Preclinical activity | |
| | therapies for heme | of JNJ-7957, a novel BCMA × CD3 bispecific | |
| | malignancies with | antibody for the treatment of multiple | |
| | Anti-CD38 antibodies | myeloma, is potentiated by daratumumab. | |
| | and survivin | 2020 Sarkar S, Chauhan . . . The CD38low | |
| | inhibitors | natural killer cell line KHYG1 transiently | |
| | 2016 WO2016209921 | expressing CD16F158V in combination | |
| | DORSHI, Parul, LO . . . | with daratumumab targets multiple | |
| | COMBINATION | myeloma cells with minimal effector NK | |
| | THERAPIES FOR | cell fratricide. | |
| | HEME | 2020 Courville EL, Yoh . . . VS38 Identifies | |
| | MALIGNANCIES WITH | Myeloma Cells With Dim CD38 Expression | |
| | ANTI-CD38 | and Plasma Cells Following Daratumumab | |
| | ANTIBODIES AND | Therapy, Which Interferes With CD38 | |
| | SURVIVIN INHIBITORS | Detection for 4 to 6 Months. | |
| | 2016 U.S. Pat. No. 10,668,149 | 2019 Luo YR, Chakrabor . . . A thin-film | |
| | Doshi, Parul (Che . . . | interferometry-based label-free | |
| | Combination | immunoassay for the detection of | |
| | therapies for heme | daratumumab interference in serum | |
| | malignancies with | protein electrophoresis. | |
| | anti-CD38 antibodies | 2019 Quelven I, Montei . . . 212Pb Alpha- | |
| | and survivin | Radioimmunotherapy targeting CD38 in | |
| | inhibitors | Multiple Myeloma: a preclinical study. | |
| | 2016 WO2016187546 | 2019 Mohan M, Weinhold . . . Daratumumab | |
| | DOSHI, Parul, SAS . . . | in high-risk relapsed/refractory multiple | |
| | ANTI-CD38 | myeloma patients: adverse effect of | |
| | ANTIBODIES FOR | chromosome 1q21 gain/amplification and | |
| | TREATMENT OF | GEP70 status on outcome. | |
| | LIGHT CHAIN | 2019 Hoylman E, Brown . . . Optimal | |
| | AMYLOIDOSIS AND | sequence of daratumumab and | |
| | OTHER CD38- | elotuzumab in relapsed and refractory | |
| | POSITIVE | multiple myeloma. | |
| | HEMATOLOGICAL | 2019 Ye Z, Wolf LA, Me . . . Risk of RBC | |
| | MALIGNANCIES | alloimmunization in multiple myeloma | |
| | 2016 | patients treated by Daratumumab. | |
| | US20170008966 | 2019 Liu L, Shurin MR, . . . A novel approach | |
| | Chaulagain, Chakr . . . | to remove interference of therapeutic | |
| | Anti-CD38 Antibodies | monoclonal antibody with serum protein | |
| | for Treatment of | electrophoresis. | |
| | Light Chain | 2019 Salas MQ, Alahmar . . . Successful | |
| | Amyloidosis and | Treatment of Refractory Red Cell Aplasia | |
| | Other CD28-Positive | after Allogeneic Hematopoietic Cell | |
| | Hematological | Transplantation with Daratumumab. | |
| | Malignancies | 2019 Scheibe F, Ostend . . . Daratumumab | |
| | 2016 U.S. Pat. No. 10,766,965 | treatment for therapy-refractory anti- | |
| | Chaulagain, Chakr . . . | CASPR2 encephalitis. | |
| | Anti-CD38 antibodies | 2019 Abdallah HM, Zhu AZX A Minimal | |
| | for treatment of light | Physiologically-Based Pharmacokinetic | |
| | chain amyloidosis | Model Demonstrates Role of the Neonatal | |
| | and other CD38- | Fc Receptor (FcRn) Competition in Drug- | |
| | positive | Disease Interactions With Antibody | |
| | hematological | Therapy. | |
| | malignancies | 2019 Even-Or E, Naser . . . Successful | |
| | 2016 WO2016133903 | treatment with daratumumab for post- | |
| | LABOTKA, Richard, . . . | HSCT refractory hemolytic anemia. | |
| | COMBINATION | 2019 Schwotzer R, Manz . . . Daratumumab | |
| | THERAPY FOR | for Relapsed or Refractory AL Amyloidosis | |
| | CANCER TREATMENT | with high Plasma Cell Burden. | |
| | 2016 | 2019 Ailawadhi S, Sher . . . Monoclonal | |
| | US20180235986 | antibody utilization characteristics in | |
| | Labotka, Richard; . . . | patients with multiple myeloma. | |
| | COMBINATION | 2019 Lovas S, Varga G, . . . Real-world data | |
| | THERAPY FOR | on the efficacy and safety of daratumumab | |
| | CANCER TREATMENT | treatment in Hungarian | |
| | 2016 | relapsed/refractory multiple myeloma | |
| | US20160237161 DE | patients. | |
| | WEERS, Michel; . . . | 2019 Jiang XY, Luider . . . Artifactual Kappa | |
| | ANTIBODIES AGAINST | Light Chain Restriction of Marrow | |
| | HUMAN CD38 | Hematogones: A Potential Diagnostic | |
| | 2016 U.S. Pat. No. 9,944,711 De | Pitfall in Minimal Residual Disease | |
| | Weers, Michel; . . . | Assessment of Plasma Cell Myeloma | |
| | Antibodies against | Patients on Daratumumab. | |
| | human CD38 | 2019 Vidal-Crespo A, M . . . Daratumumab | |
| | 2015 WO2016089960 | displays in vitro and in vivo anti-tumor | |
| | DOSHI, Parul, DA . . . | activity in models of B cell non-Hodgkin | |
| | ANTI-CD38 | lymphoma and improves responses to | |
| | ANTIBODIES FOR | standard chemo-immunotherapy | |
| | TREATMENT OF | regimens. | |
| | ACUTE MYELOID | 2019 Usmani SZ, Nahi H . . . Subcutaneous | |
| | LEUKEMIA | Delivery of Daratumumab in Relapsed or | |
| | 2015 | Refractory Multiple Myeloma. | |
| | US20160222106 | 2019 O'Dwyer M, Hender . . . CyBorD-DARA | |
| | Doshi, Parul; Dan . . . | is potent initial induction for MM and | |
| | Anti-CD38 Antibodies | enhances ADCP: initial results of the 16- | |
| | for Treatment of | BCNI-001/CTRIAL-IE 16-02 study. | |
| | Acute Myeloid | 2019 Kwun J, Matignon . . . Daratumumab | |
| | Leukemia | in Sensitized Kidney Transplantation: | |
| | 2015 U.S. Pat. No. 10,793,630 | Potentials and Limitations of Experimental | |
| | Doshi, Parul (Che . . . | and Clinical Use. | |
| | Anti-CD38 antibodies | 2019 Mizuta S, Kawata . . . VS38 as a | |
| | for treatment of | promising CD38 substitute antibody for | |
| | acute myeloid | flow cytometric detection of plasma cells | |
| | leukemia | in the daratumumab era. | |
| | 2015 | 2019 Nakamura F, Nasu R Prolonged | |
| | US20160130362 DE | severe neutropenia after the first | |
| | WEERS, Michel; . . . | daratumumab administration for multiple | |
| | ANTIBODIES AGAINST | myeloma with baseline neutropenia. | |
| | CD38 FOR | 2019 Nooka AK, Joseph . . . Clinical efficacy | |
| | TREATMENT OF | of daratumumab, pomalidomide, and | |
| | MULTIPLE MYELOMA | dexamethasone in patients with relapsed | |
| | 2015 | or refractory myeloma: Utility of re- | |
| | US20160067205 | treatment with daratumumab among | |
| | Lokhorst, Henk M . . . . | refractory patients. | |
| | Combination | 2019 Kitadate A, Kobay . . . Pretreatment | |
| | Therapies with Anti- | CD38-positive regulatory T-cells affect the | |
| | CD38 Antibodies | durable response to daratumumab in | |
| | 2015 WO2016040294 | relapsed/refractory multiple myeloma | |
| | LOKHORST, Henk | patients. | |
| | M . . . . COMBINATION | 2019 Nooka AK, Kaufman . . . Daratumumab | |
| | THERAPIES WITH | in multiple myeloma. | |
| | ANTI-CD38 | 2019 Manna A, Aulakh S . . . Targeting CD38 | |
| | ANTIBODIES | enhances the antileukemic activity of | |
| | 2015 WO2015195556 | ibrutinib in chronic lymphocytic leukemia | |
| | CHILDS, Richard, . . . | (CLL). | |
| | BLOCKING CD38 | 2019 Jullien M, Trudel . . . Single-agent | |
| | USING ANTI-CD38 | daratumumab in very advanced relapsed | |
| | F(AB′)2 TO PROTECT | and refractory multiple myeloma patients: | |
| | NK CELLS | a real-life single-center retrospective | |
| | 2015 | study. | |
| | US20180208669 | 2019 Syed YY Daratumumab: A Review in | |
| | Childs, Richard W . . . | Combination Therapy for Transplant- | |
| | BLOCKING CD38 | Ineligible Newly Diagnosed Multiple | |
| | USING ANTI-CD38 | Myeloma. | |
| | F(ab′)2 TO PROTECT | 2019 Chinoca Ziza KN, . . . A blockage | |
| | NK CELLS | monoclonal antibody protocol as an | |
| | 2015 U.S. Pat. No. 10,106,620 | alternative strategy to avoid anti-CD38 | |
| | Childs, Richard W . . . | interference in immunohematological | |
| | Blocking CD38 using | testing. | |
| | anti-CD38 F(ab′)2 to | 2019 Moore LM, Cho S, . . . MALDI-TOF | |
| | protect NK cells | mass spectrometry distinguishes | |
| | 2015 | daratumumab from M-proteins. | |
| | US20160263241 | 2019 Nahi H, Chrobok M . . . Infectious | |
| | MARKOVIC, | complications and NK cell depletion | |
| | Svetomi . . . Treating | following daratumumab treatment of | |
| | Myelomas | Multiple Myeloma. | |
| | 2015 U.S. Pat. No. 10,213,513 | 2019 Cushing MM, DeSim . . . The impact of | |
| | Markovic, Svetomi . . . | Daratumumab on transfusion service | |
| | Treating myelomas | costs. | |
| | 2015 WO2015130728 | 2018 Zhang X, Zhang C, . . . Design, synthesis | |
| | DOSHI, Parul | and evaluation of anti-CD38 antibody drug | |
| | COMBINATION | conjugate based on Daratumumab and | |
| | THERAPIES WITH | maytansinoid. | |
| | ANTI-CD38 | 2018 Adams HC, Stevena . . . High-Parameter | |
| | ANTIBODIES | Mass Cytometry Evaluation of | |
| | 2015 WO2015130732 | Relapsed/Refractory Multiple Myeloma | |
| | DOSHI, Parul ANTI- | Patients Treated with Daratumumab | |
| | CD38 ANTIBODIES | Demonstrates Immune Modulation as a | |
| | FOR TREATMENT OF | Novel Mechanism of Action. | |
| | ACUTE | 2018 Chapuy CI, Kaufma . . . Daratumumab | |
| | LYMPHOBLASTIC | for Delayed Red-Cell Engraftment after | |
| | LEUKEMIA | Allogeneic Transplantation. | |
| | 2015 | 2018 Jones RL, Mo G, B . . . Exposure- | |
| | US20150246123 | response relationship of olaratumab for | |
| | Doshi, Parul; | survival outcomes and safety when | |
| | Combination | combined with doxorubicin in patients | |
| | Therapies with Anti- | with soft tissue sarcoma. | |
| | CD38 Antibodies | 2018 Ishida T Therapeutic antibodies for | |
| | 2015 | multiple myeloma. | |
| | US20150246975 | 2018 Chehab S, Zhang C . . . Response to | |
| | Doshi, Parul; Anti- | therapeutic monoclonal antibodies for | |
| | CD38 Antibodies for | multiple myeloma in African Americans | |
| | Treatment of Acute | versus whites. | |
| | Lymphoblastic | 2018 Hosokawa M, Kashi . . . Distinct effects | |
| | Leukemia | of daratumumab on indirect and direct | |
| | 2015 U.S. Pat. No. 9,603,927 | antiglobulin tests: a new method | |
| | Doshi, Parul | employing 0.01 mol/L dithiothreitol for | |
| | Combination | negating the daratumumab interference | |
| | therapies with anti- | with preserving K antigenicity (Osaka | |
| | CD38 antibodies | method). | |
| | 2015 U.S. Pat. No. 9,732,154 | 2018 Spencer A, Lentzs . . . Daratumumab | |
| | Doshi, Parul Anti- | plus bortezomib and dexamethasone | |
| | CD38 antibodies for | versus bortezomib and dexamethasone in | |
| | treatment of acute | relapsed or refractory multiple myeloma: | |
| | lymphoblastic | updated analysis of CASTOR. | |
| | leukemia | 2018 Fedele PL, Willis . . . IMiDs through loss | |
| | 2014 | of Ikaros and Aiolos primes myeloma cells | |
| | US20150231235 VAN | for daratumumab mediated killing by | |
| | DE WINKEL, Ja . . . | upregulation of CD38. | |
| | COMBINATION | 2018 Lee AC, Greaves G . . . Bilateral Angle | |
| | TREATMENT OF | Closure Following the Infusion of a | |
| | CD38-EXPRESSING | Monoclonal Antibody to Treat Relapsing | |
| | TUMORS | Multiple Myeloma. | |
| | 2011 U.S. Pat. No. 8,486,394 | 2018 Lorenzen H, Lone . . . Thirty-three-day | |
| | Tesar, Michael; J . . . | storage of dithiothreitol-treated red blood | |
| | Generation and | cells used to eliminate daratumumab | |
| | Profiling of fully | interference in serological testing. | |
| | human hucal gold- | 2018 Tang F, Malek E, . . . Interference of | |
| | derived therapeutic | Therapeutic Monoclonal Antibodies With | |
| | antibodies specific | Routine Serum Protein Electrophoresis and | |
| | for human CD38 | Immunofixation in Patients With Myeloma: | |
| | 2011 WO2011154453 | Frequency and Duration of Detection of | |
| | WEERS, Michel de; . . . | Daratumumab and Elotuzumab. | |
| | ANTIBODIES AGAINST | 2018 Reddy K, Htut M, . . . Aseptic | |
| | HUMAN CD38 | Meningitis as a Complication of | |
| | 2011 | Daratumumab Therapy. | |
| | US20130209355 De | 2018 Kumar S, Durie B, . . . Propensity score | |
| | Weers, Michel; . . . | matching analysis to evaluate the | |
| | ANTIBODIES AGAINST | comparative effectiveness of | |
| | HUMAN CD38 | daratumumab versus real-world standard | |
| | 2011 U.S. Pat. No. 9,249,226 de | of care therapies for patients with heavily | |
| | Weers, Michel; . . . | pretreated and refractory multiple | |
| | Antibodies against | myeloma. | |
| | human CD38 | 2018 Cole S, Walsh A, . . . Integrative | |
| | 2010 | analysis reveals CD38 as a therapeutic | |
| | US20110099647 DE | target for plasma cell-rich pre-disease and | |
| | WEERS, Michel; . . . | established rheumatoid arthritis and | |
| | ANTIBODIES AGAINST | systemic lupus erythematosus. | |
| | CD38 FOR | 2018 Varga C, Maglio M . . . Current use of | |
| | TREATMENT OF | monoclonal antibodies in the treatment of | |
| | MULTIPLE MYELOMA | multiple myeloma. | |
| | 2010 U.S. Pat. No. 9,187,565 De | 2018 van de Donk NWCJ | |
| | Weers, Michel; . . . | Immunomodulatory effects of CD38- | |
| | Antibodies against | targeting antibodies. | |
| | CD38 for treatment | 2018 Wang Y, Zhang Y, . . . Fratricide of NK | |
| | of multiple myeloma | Cells in Daratumumab Therapy for | |
| | 2007 WO2008037257 | Multiple Myeloma Overcome by Ex Vivo | |
| | WINKEL, Jan van d . . . | Expanded Autologous NK Cells. | |
| | ANTI-CD38 PLUS | 2018 Zamagni E, Tacche . . . Anti-CD38 and | |
| | CORTICOSTEROIDS | anti-SLAMF7: the future of myeloma | |
| | PLUS A NON- | immunotherapy. | |
| | CORTICOSTEROID | 2018 Sigle JP, Mihm B, . . . Extending shelf | |
| | CHEMOTHERAPEUTIC | life of dithiothreitol-treated panel RBCs to | |
| | FOR TREATING | 28 days. | |
| | TUMORS | 2018 Mahaweni NM, Bos . . . Daratumumab | |
| | 2007 | augments alloreactive natural killer cell | |
| | US20100092489 Van | cytotoxicity towards CD38+ multiple | |
| | De Winkel, Ja . . . | myeloma cell lines in a biochemical context | |
| | COMBINATION | mimicking tumour microenvironment | |
| | TREATMENT OF | conditions. | |
| | CD38-EXPRESSING | 2018 Maiese EM, Ainswo . . . Comparative | |
| | TUMORS | Efficacy of Treatments for Previously | |
| | 2007 U.S. Pat. No. 9,040,050 Van | Treated Multiple Myeloma: A Systematic | |
| | De Winkel, Ja . . . | Literature Review and Network Meta- | |
| | Combination | analysis. | |
| | treatment of CD38- | 2018 Frerichs KA, Nagy . . . CD38-targeting | |
| | expressing tumors | antibodies in multiple myeloma: | |
| | 2007 | mechanisms of action and clinical | |
| | US20090076249 De | experience. | |
| | Weers, Michel; . . . | 2018 Pick M, Vainstein . . . Daratumumab | |
| | Antibodies against | resistance is frequent in advanced stage | |
| | CD38 for treatment | multiple myeloma patients irrespectively | |
| | of multiple myeloma | of CD38 express | |
| | 2006 WO2006099875 | | |
| | WEERS, Michel de; . . . | | |
| | ANTIBODIES AGAINST | | |
| | CD38 FOR | | |
| | TREATMENT OF | | |
| | MULTIPLE MYELOMA | | |
| | 2006 U.S. Pat. No. 7,829,673 De | | |
| | Weers, Michel; . . . | | |
| | Antibodies against | | |
| | CD38 for treatment | | |
| | of multiple myeloma | | |
| | 2006 | | |
| | US20090148449 De | | |
| | Weers, Michel; . . . | | |
| | Antibodies against | | |
| | cd38 for treatment of | | |
| | multiple myeloma | | |
| Encefa patent | 2020 WO2020152290 | | Encefa |
| anti-CD38 | BRESSAC, Laurence . . . | | |
| | CD31 COMPETITORS | | |
| | AND USES THEREOF | | |
| | 2020 | | |
| | US20220089765 | | |
| | BRESSAC, Laurence . . . | | |
| | CD31 COMPETITORS | | |
| | AND USES THEREOF | | |
| felzartamab | 2022 WO2023001804 | 2021 NCT05021484 Phase 2 Felzartamab in | Celgene I- |
| | HÄRTLE, Stefan | Late Antibody-Mediated Rejection | Mab |
| | TREATMENT OF | 2021 NCT05065970 Phase 2 Clinical Trial to | Biopharma |
| | ANTI-PLA2R | Assess Efficacy and Safety of the Human | Morphosys |
| | AUTOANTIBODY- | Anti-CD38 Antibody Felzartamab | |
| | MEDIATED | (MOR202) in IgA Nephropathy | |
| | MEMBRANOUS | 2021 NCT04893096 Phase 2 MOR202 for | |
| | NEPHROPATHY | Refractory MN | |
| | 2022 WO2022184676 | 2021 NCT04733040 Phase 2 Efficacy, | |
| | STEIDL, Stefan, H . . . | Safety and PK/PD of MOR202 in Anti- | |
| | ANTI-CD38 | PLA2R + Membranous Nephropathy (aMN) | |
| | ANTIBODIES FOR USE | (NewPLACE) | |
| | IN THE TREATMENT | 2019 NCT04145440 Phase 1/Phase 2 Trial | |
| | OF ANTIBODY- | to Assess Safety and Efficacy of MOR202 in | |
| | MEDIATED | Anti-PLA2R + Membranous Nephropathy | |
| | TRANSPLANT | (aMN) | |
| | REJECTION | 2019 NCT03952091 Phase 3 TJ202, | |
| | 2022 WO2022152823 | Lenalidomide and Dexamethasone vs. | |
| | STEIDL, Stefan, H . . . | Lenalidomide and Dexamethasone in | |
| | ANTI-CD38 | Subjects With Relapsed or Refractory | |
| | ANTIBODIES AND | Multiple Myeloma | |
| | THEIR USES | 2019 NCT03860038 Phase 2 TJ202 | |
| | 2021 | Combined With Dexamethasone in | |
| | US20210292431 | Subjects With Relapsed or Refractory | |
| | TESAR, Michael; J . . . | Multiple Myeloma | |
| | GENERATION AND | 2011 NCT01421186 Phase 1/Phase 2 A | |
| | PROFILING OF FULLY | Phase I/IIa Study of Human Anti-CD38 | |
| | HUMAN HuCAL | Antibody MOR03087 in | |
| | GOLD-DERIVED | Relapsed/Refractory Multiple Myeloma | |
| | THERAPEUTIC | 2023 Habicht CP, Ridde . . . Mitigation of | |
| | ANTIBODIES SPECIFIC | therapeutic anti-CD38 antibody | |
| | FOR HUMAN CD38 | interference with fab fragments: How well | |
| | 2020 WO2020187718 | does it perform? | |
| | KLUNKER, Daniel, . . . | 2022 Rovin BH, Boxhamm . . . Immunologic | |
| | ANTI-CD38 | Responses After COVID-19 Vaccination in | |
| | ANTIBODIES AND | Patients With Membranous Nephropathy | |
| | PHARMACEUTICAL | Receiving Anti-CD38 Felzartamab Therapy: | |
| | COMPOSITIONS | Results From the Phase 1b/2a M-PLACE | |
| | THEREOF FOR THE | Study. | |
| | TREATMENT OF | 2022 Mayer KA, Budde K . . . Safety, | |
| | AUTOANTIBODY- | tolerability, and efficacy of monoclonal | |
| | MEDIATED | CD38 antibody felzartamab in late | |
| | AUTOIMMUNE | antibody-mediated renal allograft | |
| | DISEASE | rejection: study protocol for a phase 2 | |
| | 2020 | trial. | |
| | US20220144965 | 2020 Jiao Y, Yi M, Xu . . . CD38: Targeted | |
| | KLUNKER, Daniel; . . . | therapy in multiple myeloma and | |
| | ANTI-CD38 | therapeutic potential for solid cancers. | |
| | ANTIBODIES AND | 2020 Raab MS, Engelhar . . . MOR202, a | |
| | PHARMACEUTICAL | novel anti-CD38 monoclonal antibody, in | |
| | COMPOSITIONS | patients with relapsed or refractory | |
| | THEREOF FOR THE | multiple myeloma: a first-in-human, | |
| | TREATMENT OF | multicentre, phase 1-2a trial. | |
| | AUTOANTIBODY- | ASCO 2011 Effect of MOR202, a human | |
| | MEDIATED | CD38 antibody, in combination with | |
| | AUTOIMMUNE | lenalidomide and bortezomib, on bone | |
| | DISEASE | lysis and tumor load in a physiologic model | |
| | 2019 WO2020120730 | of myeloma. J. Endell | |
| | BROCKS, Bodo, KEL . . . | ASCO 2016 MOR202 alone and in | |
| | ANTIBODY | combination with pomalidomide or | |
| | FORMULATIONS | lenalidomide in relapsed or refractory | |
| | 2019 | multiple myeloma: Data from clinically | |
| | US20220110872 | relevant cohorts from a phase I/IIa study | |
| | BROCKS, Bodo; KEL . . . | Marc S. Raab, Man . . . | |
| | ANTIBODY | ASH 2012 The Activity of MOR202, a Fully | |
| | FORMULATIONS | Human Anti-CD38 Antibody, Is | |
| | 2019 | Complemented by ADCP and Is | |
| | US20200079871 | Synergistically Enhanced by Lenalidomide | |
| | LeClair, Stephane . . . | in Vitro and in Vivo Jan Endell, Raine . . . | |
| | TREATMENT FOR | ASH 2015 Phase I/IIa Study of the Human | |
| | MULTIPLE MYELOMA | Anti-CD38 Antibody MOR202 (MOR03087) | |
| | (MM) | in Relapsed or Refractory Multiple | |
| | 2018 | Myeloma Marc S Raab, MD, . . . | |
| | US20190077877 | ASH 2015 MOR202, a Human Anti-CD38 | |
| | TESAR, MICHAEL; J . . . | Monoclonal Antibody, Mediates Potent | |
| | GENERATION AND | Tumoricidal Activity In Vivo and Shows | |
| | PROFILING OF FULLY | Synergistic Efficacy in Combination with | |
| | HUMAN HuCAL | Different Antineoplastic Compounds | |
| | GOLD-DERIVED | Rainer Boxhammer, . . . | |
| | THERAPEUTIC | | |
| | ANTIBODIES SPECIFIC | | |
| | FOR HUMAN CD38 | | |
| | 2018 U.S. Pat. No. 11,059,902 | | |
| | Tesar, Michael (F . . . | | |
| | Generation and | | |
| | profiling of fully | | |
| | human HuCAL GOLD- | | |
| | derived therapeutic | | |
| | antibodies specific | | |
| | for human CD38 | | |
| | 2018 | | |
| | US20180215832 | | |
| | TESAR, MICHAEL; J . . . | | |
| | ANTI-CD38 HUMAN | | |
| | ANTIBODIES AND | | |
| | USES THEREFOR | | |
| | 2017 | | |
| | US20180022823 | | |
| | ROJKJAER, LISA; B . . . | | |
| | METHOD FOR THE | | |
| | TREATMENT OF | | |
| | MULTIPLE MYELOMA | | |
| | OR NON-HODGKINS | | |
| | LYMPHOMA WITH | | |
| | ANTI-CD38 | | |
| | ANTIBODY AND | | |
| | BORTEZOMIB OR | | |
| | CARFILZOMIB | | |
| | 2017 U.S. Pat. No. 10,308,722 | | |
| | Rojkjaer, Lisa (H . . . | | |
| | Method for the | | |
| | treament of multiple | | |
| | myeloma or non- | | |
| | hodgkins lymphoma | | |
| | with anti-CD38 | | |
| | antibody in | | |
| | combination with | | |
| | thalidomine, | | |
| | lenalidomids, or | | |
| | pomalidomide | | |
| | 2017 WO2018015498 | | |
| | BENJAMIN, Susan, . . . | | |
| | COMBINATIONS OF | | |
| | INECALCITOL WITH | | |
| | AN ANTI-CD38 | | |
| | AGENT AND THEIR | | |
| | USES FOR TREATING | | |
| | CANCER | | |
| | 2017 WO2017149122 | | |
| | HÄRTLE, Stefan | | |
| | CLINICAL | | |
| | ASSESSMENT OF M- | | |
| | PROTEIN RESPONSE | | |
| | IN MULTIPLE | | |
| | MYELOMA | | |
| | 2017 | | |
| | US20190077875 | | |
| | Hartle, Stefan; | | |
| | CLINICAL | | |
| | ASSESSMENT OF M- | | |
| | PROTEIN RESPONSE | | |
| | IN MULTIPLE | | |
| | MYELOMA | | |
| | 2016 WO2016180958 | | |
| | LECLAIR, Stéphane . . . | | |
| | TREATMENT FOR | | |
| | MULTIPLE MYELOMA | | |
| | (MM) | | |
| | 2016 | | |
| | US20190048091 | | |
| | LeClair, Stephane . . . | | |
| | TREATMENT FOR | | |
| | MULTIPLE MYELOMA | | |
| | (MM) | | |
| | 2016 U.S. Pat. No. 10,533,057 | | |
| | LeClair, Stephane . . . | | |
| | Treatment for | | |
| | multiple myeloma | | |
| | (MM) | | |
| | 2016 | | |
| | US20160200828 | | |
| | Tesar, Michael; J . . . | | |
| | ANTI-CD38 HUMAN | | |
| | ANTIBODIES AND | | |
| | USES THEREOF | | |
| | 2016 | | |
| | US20160222127 | | |
| | Rojkjaer, Lisa; B . . . | | |
| | Anti-CD38 Antibody | | |
| | and Lenalidomide or | | |
| | Bortezomib for the | | |
| | Treatment of Multipe | | |
| | Myeloma and NHL | | |
| | 2016 U.S. Pat. No. 9,765,152 | | |
| | Rojkjaer, Lisa; B . . . | | |
| | Method for the | | |
| | treatment of multiple | | |
| | myeloma or non- | | |
| | Hodgkins lymphoma | | |
| | with anti-CD38 | | |
| | antibody and | | |
| | bortezomib or | | |
| | carfilzomib | | |
| | 2015 | | |
| | US20160096901 | | |
| | Tesar, Michael; J . . . | | |
| | ANTI-CD38 HUMAN | | |
| | ANTIBODIES AND | | |
| | USES THEREOF | | |
| | 2015 | | |
| | US20160115243 | | |
| | Tesar, Michael; J . . . | | |
| | ANTI-CD38 HUMAN | | |
| | ANTIBODIES AND | | |
| | USES THEREOF | | |
| | 2015 | | |
| | US20160075796 | | |
| | Tesar, Michael; J . . . | | |
| | Generation and | | |
| | profiling of fully | | |
| | human HuCAL GOLD- | | |
| | derived therapeutic | | |
| | antibodies specific | | |
| | for human CD38 | | |
| | 2015 U.S. Pat. No. 10,184,005 | | |
| | Tesar, Michael (F . . . | | |
| | Generation and | | |
| | profiling of fully | | |
| | human HuCAL GOLD- | | |
| | derived therapeutic | | |
| | antibodies specific | | |
| | for human CD38 | | |
| | 2015 | | |
| | US20150232571 | | |
| | Tesar, Michael; J . . . | | |
| | ANTI-CD38 HUMAN | | |
| | ANTIBODIES AND | | |
| | USES THEREFOR | | |
| | 2014 | | |
| | US20150017160 | | |
| | Rojkjaer, Lisa; B . . . | | |
| | COMBINATIONS AND | | |
| | USES THEREOF | | |
| | 2014 U.S. Pat. No. 9,289,490 | | |
| | Rojkjaer, Lisa; B . . . | | |
| | Combinations and | | |
| | uses thereof | | |
| | 2013 WO2014068114 | | |
| | ESSLER, Markus | | |
| | RADIOLABELLED | | |
| | ANTIBODY AND USES | | |
| | THEREOF | | |
| | 2013 | | |
| | US20150283275 | | |
| | Essler, Markus; | | |
| | Radiolabelled | | |
| | Antibody And Uses | | |
| | Thereof | | |
| | 2013 U.S. Pat. No. 9,486,547 | | |
| | Essler, Markus | | |
| | Radiolabelled | | |
| | antibody and uses | | |
| | thereof | | |
| | 2013 WO2014048921 | | |
| | ENDELL, Jan, ROJK . . . | | |
| | COMBINATIONS AND | | |
| | USES THEREOF | | |
| | 2013 | | |
| | US20150238603 | | |
| | Endell, Jan; Rojk . . . | | |
| | COMBINATIONS AND | | |
| | USES THEREOF | | |
| | 2013 U.S. Pat. No. 9,579,378 | | |
| | Endell, Jan; Rojk . . . | | |
| | Combinations and | | |
| | uses thereof | | |
| | 2013 | | |
| | US20130273072 | | |
| | Tesar, Michael; J . . . | | |
| | FULLY HUMAN | | |
| | THERAPEUTIC | | |
| | ANTIBODIES SPECIFIC | | |
| | FOR HUMAN CD38 | | |
| | 2013 U.S. Pat. No. 9,193,799 | | |
| | Tesar, Michael; J . . . | | |
| | Fully human | | |
| | therapeutic | | |
| | antibodies specific | | |
| | for human CD38 | | |
| | 2011 | | |
| | US20120076782 | | |
| | Tesar, Michael; S . . . | | |
| | GENERATION AND | | |
| | PROFILING OF FULLY | | |
| | HUMAN HUCAL | | |
| | GOLD.RTM.-DERIVED | | |
| | THERAPEUTIC | | |
| | ANTIBODIES SPECIFIC | | |
| | FOR HUMAN CD38 | | |
| | 2011 U.S. Pat. No. 9,200,061 | | |
| | Tesar, Michael; J . . . | | |
| | Generation and | | |
| | profiling of fully | | |
| | human HuCAL gold ®- | | |
| | derived therapeutic | | |
| | antibodies specific | | |
| | for human CD3i | | |
| | 2011 | | |
| | US20120052078 | | |
| | Tesar, Michael; J . . . | | |
| | GENERATION AND | | |
| | PROFILING OF FULLY | | |
| | HUMAN HUCAL | | |
| | GOLD-DERIVED | | |
| | THERAPEUTIC | | |
| | ANTIBODIES SPECIFIC | | |
| | FOR HUMAN CD38 | | |
| | 2011 WO2012041800 | | |
| | ROJKJAER, Lisa, B . . . | | |
| | ANTI-CD38 | | |
| | ANTIBODY AND | | |
| | LENALIDOMIDE OR | | |
| | BORTEZOMIB FOR | | |
| | THE TREATMENT OF | | |
| | MULTIPLE MYELOMA | | |
| | AND NHL | | |
| | 2011 | | |
| | US20130302318 | | |
| | Rojkjaer, Lisa; B . . . | | |
| | Anti-CD38 Antibody | | |
| | and Lenalidomide or | | |
| | Bortezomib for the | | |
| | Treatment of Multipe | | |
| | Myeloma and NHL | | |
| | 2011 U.S. Pat. No. 8,877,899 | | |
| | Rojkjaer, Lisa; B . . . | | |
| | Anti-CD38 antibody | | |
| | and lenalidomide or | | |
| | bortezomib for the | | |
| | treatment of multipe | | |
| | myeloma and NHL | | |
| | 2006 WO2007042309 | | |
| | TESAR, Michael; | | |
| | GENERATION AND | | |
| | PROFILING OF FULLY | | |
| | HUMAN HUCAL | | |
| | GOLD-DERIVED | | |
| | THERAPEUTIC | | |
| | ANTIBODIES SPECIFIC | | |
| | FOR HUMAN CD38 | | |
| | 2006 | | |
| | US20090252733 | | |
| | Tesar, Michael; | | |
| | Generation and | | |
| | Profiling of Fully | | |
| | Human Gold-Derived | | |
| | Therapeutic | | |
| | Antibodies Specific | | |
| | for Human CD38 | | |
| | 2006 U.S. Pat. No. 8,088,896 | | |
| | Tesar, Michael; J . . . | | |
| | Generation and | | |
| | profiling of fully | | |
| | human gold-derived | | |
| | therapeutic | | |
| | antibodies specific | | |
| | for human CD38 | | |
| | 2006 WO2006125640 | | |
| | TESAR, Michael; | | |
| | GENERATION AND | | |
| | PROFILING OF FULLY | | |
| | HUMAN HUCAL | | |
| | GOLD ®-DERIVED | | |
| | THERAPEUTIC | | |
| | ANTIBODIES SPECIFIC | | |
| | FOR HUMAN CD38 | | |
| | 2006 | | |
| | US20090123950 | | |
| | Tesar, Michael; | | |
| | Generation And | | |
| | Profiling Of Fully | | |
| | Human Hucal | | |
| | Gold.RTM.-Derived | | |
| | Therapeutic | | |
| | Antibodies Specific | | |
| | For Human CD38 | | |
| | 2005 WO2005103083 | | |
| | TESAR, Michael; J . . . | | |
| | ANTI-CD38 HUMAN | | |
| | ANTIBODIES AND | | |
| | USES THEREFOR | | |
| FTL004 | | 2022 Zhang G, Guo C, W . . . FTL004, an anti- | Sound |
| | | CD38 mAb with negligible RBC binding and | Biologics |
| | | enhanced pro-apoptotic activity, is a novel | |
| | | candidate for treatments of multiple | |
| | | myeloma and non-Hodgkin lymphoma. | |
| GEN3014 | | 2021 NCT04824794 Phase 1/Phase 2 | Genmab |
| | | GEN3014 Safety Trial in Relapsed or | |
| | | Refractory Hematologic Malignancies | |
| Genmab anti- | | 2016 Drent E, Groen RW . . . Pre-clinical | Genmab VU |
| CD38 CAR | | evaluation of CD38 chimeric antigen | U. Med. Center |
| | | receptor engineered T cells for the | |
| | | treatment of multiple myeloma. | |
| HLX15 | | 2023 NCT05679258 Phase 1 A Study to | Shanghai |
| | | Compare the PK, Safety, Tolerability, and | Henlius |
| | | Immunogenicity of HLX15 With | |
| | | Daratumumab in Male Subjects | |
| isatuximab-irfc | 2022 | 2022 NCT05559827 Phase 2 Efficacy of the | ImmunoGen |
| | US20220275102 | antiCD38 Monoclonal Antibody Isatuximab | Sanofi |
| | CAMERON, | in the Treatment of PCRA by Major ABO | |
| | Beatrice . . . ANTI-CD38 | Mismatch After Allogeneic Hematopoietic | |
| | ANTIBODIES AND | Stem Cell Transplantation | |
| | METHODS OF USE | 2022 NCT05405166 Phase 3 SC Versus IV | |
| | 2021 WO2022099257 | Isatuximab in Combination With | |
| | SEMIOND, | Pomalidomide and Dexamethasone in | |
| | Dorothee . . . USE OF | RRMM | |
| | ISATUXIMAB FOR | 2021 NCT04883242 Phase 2 Isatuximab, | |
| | THE TREATMENT OF | Carfilzomib, Pomalidomide, and | |
| | MULTIPLE MYELOMA | Dexamethasone for the Treatment of | |
| | 2021 | Relapsed or Refractory Multiple Myeloma | |
| | US20220041746 | 2021 NCT04850599 Phase 2 Isatuximab, | |
| | CAMERON, | Carfilzomib, and Pomalidomide for the | |
| | Beatrice . . . ANTI-CD38 | Treatment of Relapsed or Refractory | |
| | ANTIBODIES AND | Multiple Myeloma | |
| | METHODS OF USE | 2021 NCT04802031 Phase 2 Rapid-infusion | |
| | 2021 U.S. Pat. No. 11,365,261 | Isatuximab in Relapsed/Refractory | |
| | Cameron, Beatrice . . . | Multiple Myeloma | |
| | Anti-CD38 antibodies | 2021 NCT04754945 Phase 1 Isatuximab as | |
| | and methods of use | Upfront Therapy for the Treatment of High | |
| | 2021 | Risk AL Amyloidosis | |
| | US20220088194 | 2021 NCT04558931 Phase 2 Clinical Trial | |
| | TOMKINSON, | for Autologus NK Cells Alone or in | |
| | Blake; . . . | Combination With Isatuximab as | |
| | COMPOSITIONS | Maintenance for Multiple Myeloma | |
| | COMPRISING ANTI- | 2021 NCT04643002 Phase 1/Phase 2 | |
| | CD38 ANTIBODIES | Isatuximab in Combination With Novel | |
| | AND CARFILZOMIB | Agents in RRMM | |
| | 2020 | 2020 NCT04614558 Phase 2 Isatuximab in | |
| | US20210171653 | Patients With Monoclonal Gammopathy of | |
| | RISSE, Marie-Laur . . . | Renal Significance | |
| | USE OF ISATUXIMAB | 2020 NCT04240054 Phase 2 Study of | |
| | FOR THE TREATMENT | Bortezomib, Isatuximab, | |
| | OF RELAPSED | Cyclophosphamide and Dexamethasone | |
| | AND/OR | (VICD) Induction in Transplant-Eligible | |
| | REFRACTORY | Multiple Myeloma Patients | |
| | MULTIPLE MYELOMA | 2020 NCT04270409 Phase 3 Isatuximab in | |
| | 2020 WO2021113739 | Combination With Lenalidomide and | |
| | BALLET, Thomas, B . . . | Dexamethasone in High-risk Smoldering | |
| | FORMULATIONS OF | Multiple Myeloma | |
| | ANTI-CD38 | 2020 NCT04340076 Phase 4 Dose | |
| | ANTIBODIES FOR | Reduction of IL17 and IL23 Inhibitors in | |
| | SUBCUTANEOUS | Psoriasis | |
| | ADMINISTRATION | 2020 NCT04294459 Phase 1/Phase 2 | |
| | 2020 WO2021113754 | Safety, Pharmacokinetics, and Preliminary | |
| | RISSE, Marie-Laur . . . | Efficacy of Isatuximab in Patients Awaiting | |
| | USE OF ISATUXIMAB | Kidney Transplantation | |
| | FOR THE TREATMENT | 2019 NCT03860844 Phase 2 Isatuximab in | |
| | OF RELAPSED | Combination With Chemotherapy in | |
| | AND/OR | Pediatric Patients With | |
| | REFRACTORY | Relapsed/Refractory Acute Lymphoblastic | |
| | MULTIPLE MYELOMA | Leukemia or Acute Myeloid Leukemia | |
| | 2020 | 2019 NCT04045795 Phase 1 Multi-center, | |
| | US20210188996 | Open-label, Phase 1b Study in Patients | |
| | Huille, Sylvain; . . . | With Relapsed/Refractory Multiple | |
| | FORMULATIONS OF | Myeloma (RRMM) | |
| | ANTI-CD38 | 2019 NCT03869190 Phase 1/Phase 2 A | |
| | ANTIBODIES FOR | Study Evaluating the Efficacy and Safety of | |
| | SUBCUTANEOUS | Multiple Immunotherapy-based Treatment | |
| | ADMINISTRATION | Combinations in Patients With Locally | |
| | 2020 | Advanced or Metastatic Urothelial | |
| | US20200399391 | Carcinoma After Failure With Platinum- | |
| | CHIRON BLONDEL, | Containing Chemotherapy | |
| | M . . . ANTI-CD38 | 2018 NCT03769181 Phase 1/Phase 2 A | |
| | ANTIBODIES AND | Study of Isatuximab-based Therapy in | |
| | FORMULATIONS | Participants With Lymphoma | |
| | 2020 WO2020232173 | 2018 NCT03733717 Phase 1 Evaluation of | |
| | CAMPANA | Pharmacokinetics, Safety, and Preliminary | |
| | ZAMBRANO, . . . | Efficacy of Isatuximab in Chinese Patients | |
| | METHODS OF | With Relapsed and/or Refractory Multiple | |
| | ADMINISTERING | Myeloma | |
| | ANTI-CD38 | 2018 NCT03617731 Phase 3 Trial on the | |
| | ANTIBODY TO TREAT | Effect of Isatuximab to | |
| | MULTIPLE MYELOMA | Lenalidomide/Bortezomib/Dexamethasone | |
| | 2020 | (RVd) Induction and Lenalidomide | |
| | US20210171650 | Maintenance in Patients With Newly | |
| | AUDAT, Heloise; M . . . | Diagnosed Myeloma (GMMG HD7) | |
| | METHODS OF | 2018 NCT03555149 Phase 1/Phase 2 A | |
| | ADMINISTERING | Study Evaluating the Efficacy and Safety of | |
| | ANTI-CD38 | Multiple Immunotherapy-Based Treatment | |
| | ANTIBODY | Combinations in Patients With Metastatic | |
| | 2020 WO2020219681 | Colorectal Cancer (Morpheus-CRC) | |
| | CAMERON, | 2018 NCT03637764 Phase 1/Phase 2 | |
| | Béatrice . . . ANTI-CD38 | Safety, Preliminary Efficacy and PK of | |
| | ANTIBODIES AND | Isatuximab (SAR650984) Alone or in | |
| | FORMULATIONS | Combination With Atezolizumab in | |
| | 2020 | Patients With Advanced Malignancies | |
| | US20200408765 | 2018 NCT03499808 Phase 2 S1702 | |
| | PARK, Peter U.; B . . . | Isatuximab in Treating Patients With | |
| | NOVEL ANTI-CD38 | Relapsed or Refractory Primary | |
| | ANTIBODIES FOR THE | Amyloidosis | |
| | TREATMENT OF | 2017 NCT03275285 Phase 3 Multinational | |
| | CANCER | Clinical Study Comparing Isatuximab, | |
| | 2020 WO2020160020 | Carfilzomib And Dexamethasone To | |
| | CAMPANA | Carfilzomib And Dexamethasone In | |
| | ZAMBRANO, . . . | Relapse And/Or Refractory Multiple | |
| | METHODS OF | Myeloma Patients | |
| | TREATING MULTIPLE | 2017 NCT03104842 Phase 2 Evaluation | |
| | MYELOMA | iNduction, Consolidation and Maintenance | |
| | 2019 WO2020012383 | Treatment With Isatuximab, Carfilzomib, | |
| | ADRIAN, Francisco . . . | LEnalidomide and Dexamethasone | |
| | COMBINATION | 2017 NCT03194867 Phase 1/Phase 2 | |
| | THERAPIES AGAINST | Isatuximab in Combination With | |
| | CANCER TARGETING | REGN2810 in Relapsed/Refractory Multiple | |
| | CD38 AND TGF-BETA | Myeloma (RRMM) Patients | |
| | 2019 | 2017 NCT02960555 Phase 2 Trial of | |
| | US20210155708 | Isatuximab (SAR650984) in Patients With | |
| | ADRIAN, Francisco . . . | Intermediate and High Risk Smoldering | |
| | COMBINATION | Multiple Myeloma | |
| | THERAPIES AGAINST | 2017 NCT02999633 Phase 2 Safety and | |
| | CANCER TARGETING | Efficacy of Isatuximab in Lymphoblastic | |
| | CD38 AND TGF-BETA | Leukemia | |
| | 2019 | 2016 NCT02990338 Phase 3 Multinational | |
| | US20190321471 | Clinical Study Comparing Isatuximab, | |
| | TOMKINSON, | Pomalidomide, and Dexamethasone to | |
| | Blake; . . . | Pomalidomide and Dexamethasone in | |
| | COMPOSITIONS | Refractory or Relapsed and Refractory | |
| | COMPRISING ANTI- | Multiple Myeloma Patients | |
| | CD38 ANTIBODIES | 2016 NCT02812706 Phase 1/Phase 2 | |
| | AND CARFILZOMIB | Isatuximab Single Agent Study in Japanese | |
| | 2018 | Relapsed AND Refractory Multiple | |
| | US20190284294 | Myeloma Patients | |
| | DESLANDES, Antoin . . . | 2015 NCT02514668 Phase 1 A Study to | |
| | SPECIFIC ANTI-CD38 | Evaluate the Safety and Pharmacokinetics | |
| | ANTIBODIES FOR | of SAR650984 in Patients With Multiple | |
| | TREATING HUMAN | Myeloma | |
| | CANCERS | 2015 NCT02513186 Phase 1 SAR650984 | |
| | 2018 | Combined to CyBorD in Newly Diagnosed | |
| | US20190106504 WU, | Multiple Myeloma (MM) Non Eligible for | |
| | Lan; XU, Ling . . . ANTI- | Transplant | |
| | CD38 ANTIBODIES | 2014 NCT02283775 Phase 1 SAR650984, | |
| | AND METHODS OF | Pomalidomide and Dexamethasone in | |
| | USE | Combination in RRMM Patients | |
| | 2018 WO2019074973 | 2014 NCT02332850 Phase 1 SAR650984 in | |
| | WU, Lan, XU, Ling . . . | Combination With Carfilzomib for | |
| | ANTI-CD38 | Treatment of Relapsed or Refractory | |
| | ANTIBODIES AND | Multiple Myeloma | |
| | METHODS OF USE | 2012 NCT01749969 Phase 1 Phase 1 | |
| | 2018 U.S. Pat. No. 11,186,649 | SAR650984 Combination With | |
| | Wu, Lan (Bridgewa . . . | Lenalidomide | |
| | Anti-CD38 antibodies | 2010 NCT01084252 Phase 1/Phase 2 Dose | |
| | and methods of use | Escalation Study of Anti-CD38 Monoclonal | |
| | 2017 WO2018015498 | Antibody in Patients With Selected CD38+ | |
| | BENJAMIN, Susan, . . . | Hematological Malignancies | |
| | COMBINATIONS OF | 2023 Habicht CP, Ridde . . . Mitigation of | |
| | INECALCITOL WITH | therapeutic anti-CD38 antibody | |
| | AN ANTI-CD38 | interference with fab fragments: How well | |
| | AGENT AND THEIR | does it perform? | |
| | USES FOR TREATING | 2022 Djebbari F, Poynt . . . Outcomes of anti- | |
| | CANCER | CD38 isatuximab plus pomalidomide and | |
| | 2017 | dexamethasone in five relapsed myeloma | |
| | US20180022814 | patients with prior exposure to anti-C38 | |
| | Deslandes, Antoin . . . | daratumumab: case series. | |
| | SPECIFIC ANTI-CD38 | 2022 Djebbari F, Rampo . . . Infections in | |
| | ANTIBODIES FOR | relapsed myeloma patients treated with | |
| | TREATING HUMAN | isatuximab plus pomalidomide and | |
| | CANCERS | dexamethasone during the COVID-19 | |
| | 2017 | pandemic: Initial results of a UK-wide real- | |
| | US20170343550 | world study. | |
| | PARK, Peter U.; B . . . | 2022 Sunami K, Fuchida . . . Anti-CD38 | |
| | NOVEL ANTI-CD38 | antibody isatuximab monotherapy for | |
| | ANTIBODIES FOR THE | Japanese individuals with | |
| | TREATMENT OF | relapsed/refractory multiple myeloma: An | |
| | CANCER | update of the phase 1/2 ISLANDs study. | |
| | 2016 | 2022 Martin TG, Capra . . . Isatuximab plus | |
| | US20170106085 | Carfilzomib and Dexamethasone vs | |
| | TOMKINSON, | Carfilzomib and Dexamethasone in | |
| | Blake; . . . | Patients with Relapsed Multiple Myeloma: | |
| | COMPOSITIONS | IKEMA Subgroup Analysis by Prior | |
| | COMPRISING ANTI- | Transplantation. | |
| | CD38 ANTIBODIES | 2022 Goldschmidt H, Ma . . . Addition of | |
| | AND CARFILZOMIB | isatuximab to lenalidomide, bortezomib, | |
| | 2016 | and dexamethasone as induction therapy | |
| | US20170056496 | for newly diagnosed, transplantation- | |
| | LEJEUNE, Pascale; . . . | eligible patients with multiple myeloma | |
| | ANTITUMOR | (GMMG-HD7): part 1 of an open-label, | |
| | COMBINATIONS | multicentre, randomised, active- | |
| | CONTAINING | controlled, phase 3 trial. | |
| | ANTIBODIES | 2022 Carlo-Stella C, Z . . . A phase 1/2, open- | |
| | RECOGNIZING | label, multicenter study of isatuximab in | |
| | SPECIFICALLY CD38 | combination with cemiplimab in patients | |
| | AND | with lymphoma. | |
| | CYCLOPHOSPHAMIDE | 2022 Chami B, Okuda M, . . . Anti-CD38 | |
| | 2014 | monoclonal antibody interference with | |
| | US20150118251 | blood compatibility testing: Differentiating | |
| | Deslandes, Antoin . . . | isatuximab and daratumumab via | |
| | SPECIFIC ANTI-CD38 | functional epitope mapping. | |
| | ANTIBODIES FOR | 2022 Martin T, Dimopou . . . MM-064 | |
| | TREATING HUMAN | Updated Progression-Free Survival and | |
| | CANCERS | Depth of Response in IKEMA, a | |
| | 2014 WO2015066450 | Randomized Phase 3 Trial of Isatuximab, | |
| | DESLANDES, Antoin . . . | Carfilzomib, and Dexamethasone (Isa-Kd) | |
| | SPECIFIC ANTI-CD38 | Versus Kd in Relapsed Multiple Myeloma. | |
| | ANTIBODIES FOR | 2022 Quach H, Parmar G . . . MM-071 | |
| | TREATING HUMAN | Subcutaneous (SC) Isatuximab (Isa) | |
| | CANCERS | Administration by an On-Body Delivery | |
| | 2014 WO2014159911 | System (OBDS) in Combination With | |
| | TOMKINSON, Blake, . . . | Pomalidomide-Dexamethasone (Pd) in | |
| | COMPOSITIONS | Relapsed/Refractory Multiple Myeloma | |
| | COMPRISING ANTI- | (RRMM) Patients: Interim Phase 1b Study | |
| | CD38 ANTIBODIES | Results. | |
| | AND CARFILZOMIB | 2022 Manasanch EE, Bek . . . MM-086 Real- | |
| | 2014 | World Experience With Isatuximab (Isa) in | |
| | US20160022813 | Patients With Relapsed and/or Refractory | |
| | TOMKINSON, | Multiple Myeloma: IONA-MM First Interim | |
| | Blake; . . . | Analysis. | |
| | COMPOSITIONS | 2022 Martin T, Hajek R . . . MM-188 Depth of | |
| | COMPRISING ANTI- | Response of Isatuximab plus Carfilzomib | |
| | CD38 ANTIBODIES | and Dexamethasone in Relapsed Multiple | |
| | AND CARFILZOMIB | Myeloma: IKEMA Updated Analysis. | |
| | 2013 | 2022 Beksac M, Spicka . . . Evaluation of | |
| | US20140161819 | isatuximab in patients with soft-tissue | |
| | Hann, Byron C.; T . . . | plasmacytomas: An analysis from ICARIA- | |
| | Compositions | MM and IKEMA. | |
| | Comprising Anti- | 2022 Spicka I, Moreau . . . Isatuximab plus | |
| | CD38 Antibodies and | carfilzomib and dexamethasone in | |
| | Lenalidomide | relapsed multiple myeloma patients with | |
| | 2013 U.S. Pat. No. 10,342,869 | high-risk cytogenetics: IKEMA subgroup | |
| | Hann, Byron C. (S . . . | analysis. | |
| | Compositions | 2022 Simonelli M, Garr . . . Isatuximab plus | |
| | comprising anti-CD38 | atezolizumab in patients with advanced | |
| | antibodies and | solid tumors: results from a phase I/II, | |
| | lenalidomide | open-label, multicenter study. | |
| | 2012 | 2022 Martin T, Mikhael . . . Depth of | |
| | US20120156218 | response and response kinetics of | |
| | PARK, Peter U.; B . . . | isatuximab plus carfilzomib and | |
| | NOVEL ANTI-CD38 | dexamethasone in relapsed multiple | |
| | ANTIBODIES FOR THE | myeloma. | |
| | TREATMENT OF | 2022 El-Shershaby HM, . . . Radiolabeling | |
| | CANCER | and Cytotoxicity of Monoclonal Antibody | |
| | 2011 WO2012076663 | Isatuximab Functionalized Silver | |
| | DECKERT, Jutta, L . . . | Nanoparticles on the Growth of Multiple | |
| | ANTITUMOR | Myeloma. | |
| | COMBINATIONS | 2022 Shah B, Gray J, A . . . Pharmacy | |
| | CONTAINING | considerations: Use of anti-CD38 | |
| | ANTIBODIES | monoclonal antibodies in relapsed and/or | |
| | RECOGNIZING | refractory multiple myeloma. | |
| | SPECIFICALLY CD38 | 2022 Facon T, Moreau P . . . Isatuximab plus | |
| | AND BORTEZOMIB | carfilzomib and dexamethasone versus | |
| | 2011 | carfilzomib and dexamethasone in elderly | |
| | US20140186337 | patients with relapsed multiple myeloma: | |
| | Deckert, Jutta; L . . . | IKEMA subgroup analysis. | |
| | Antitumors | 2022 Capra M, Martin T . . . Isatuximab plus | |
| | Combinations | carfilzomib and dexamethasone versus | |
| | Containing | carfilzomib and dexamethasone in | |
| | Antibodies | relapsed multiple myeloma patients with | |
| | Recognizing | renal impairment: IKEMA subgroup | |
| | Specifically CD38 And | analysis. | |
| | Bortezomib | 2022 Kim K, Min CK, Ko . . . Isatuximab plus | |
| | 2011 U.S. Pat. No. 9,314,522 | carfilzomib and dexamethasone in East | |
| | Deckert, Jutta; L . . . | Asian patients with relapsed multiple | |
| | Antitumors | myeloma: IKEMA subgroup analysis. | |
| | combinations | 2022 Gozzetti A, Ciofi . . . Anti CD38 | |
| | containing antibodies | monoclonal antibodies for multiple | |
| | recognizing | myeloma treatment. | |
| | specifically CD38 and | 2022 Sunami K, Ikeda T . . . Isatuximab- | |
| | bortezomib | Pomalidomide-Dexamethasone Versus | |
| | 2011 | Pomalidomide-Dexamethasone in East | |
| | US20110262454 | Asian Patients With Relapsed/Refractory | |
| | PARK, Peter U.; B . . . | Multiple Myeloma: ICARIA-MM Subgroup | |
| | NOVEL ANTI-CD38 | Analysis. | |
| | ANTIBODIES FOR THE | 2022 Rachedi F, Koiwai . . . Exposure- | |
| | TREATMENT OF | Response Analyses for | |
| | CANCER | Selection/Confirmation of Optimal | |
| | 2009 WO2010061358 | Isatuximab Dosing Regimen in | |
| | LEJEUNE, Pascale; . . . | Combination With | |
| | ANTITUMOR | Pomalidomide/Dexamethasone Treatment | |
| | COMBINATIONS | in Patients With Multiple Myeloma. | |
| | CONTAINING | 2022 Kassem S, Diallo . . . SAR442085, a | |
| | ANTIBODIES | novel anti-CD38 antibody with enhanced | |
| | RECOGNIZING | antitumor activity against multiple | |
| | SPECIFICALLY CD38 | myeloma. | |
| | AND VINCRISTINE | 2022 Boissel N, Cheval . . . Isatuximab | |
| | 2009 WO2010061357 | monotherapy in patients with refractory T- | |
| | LEJEUNE, Pascale; . . . | acute lymphoblastic leukemia or T- | |
| | ANTITUMOR | lymphoblastic lymphoma: Phase 2 study. | |
| | COMBINATIONS | 2022 Dimopoulos MA, Ri . . . Treatment | |
| | CONTAINING | Options for Patients With Heavily | |
| | ANTIBODIES | Pretreated Relapsed and Refractory | |
| | RECOGNIZING | Multiple Myeloma. | |
| | SPECIFICALLY CD38 | 2021 Wheeler RD, Costa . . . Case report: | |
| | AND MELPHALAN | Interference from isatuximab on serum | |
| | 2009 WO2010061359 | protein electrophoresis prevented | |
| | LEJEUNE, Pascale; . . . | demonstration of complete remission in a | |
| | ANTITUMOR | myeloma patient. | |
| | COMBINATIONS | 2021 Wilmoth J, Colson . . . Isatuximab: | |
| | CONTAINING | Nursing Considerations for Use in the | |
| | ANTIBODIES | Treatment of Multiple Myeloma. | |
| | RECOGNIZING | 2021 Muccio S, Taverni . . . Validated | |
| | SPECIFICALLY CD38 | Method Based on Immunocapture and | |
| | AND CYTARABINE | Liquid Chromatography Coupled to High- | |
| | 2009 WO2010061360 | Resolution Mass Spectrometry to Eliminate | |
| | LEJEUNE, Pascale; . . . | Isatuximab Interference with M-Protein | |
| | ANTITUMOR | Measurement in Serum. | |
| | COMBINATIONS | 2021 Takakuwa T, Ohta . . . Isatuximab plus | |
| | CONTAINING | Pomalidomide and Dexamethasone in a | |
| | ANTIBODIES | Patient with Dialysis-Dependent Multiple | |
| | RECOGNIZING | Myeloma. | |
| | SPECIFICALLY CD38 | 2021 Thai HT, Gaudel-D . . . Joint modeling | |
| | AND | and simulation of M-protein dynamics and | |
| | CYLOPHOSPHAMIDE | progression-free survival for alternative | |
| | 2009 | isatuximab dosing with | |
| | US20110274686 | pomalidomide/dexamethasone. | |
| | Lejeune, Pascale; . . . | 2021 Hulin C, Beksac M . . . Antibody | |
| | ANTITUMOR | interference and response kinetics of | |
| | COMBINATIONS | isatuximab plus pomalidomide and | |
| | CONTAINING | dexamethasone in multiple myeloma. | |
| | ANTIBODIES | 2021 Banerjee R, Lo M, . . . Isatuximab, | |
| | RECOGNIZING | carfilzomib and dexamethasone (Isa-Kd) | |
| | SPECIFICALLY CD38 | for the management of relapsed multiple | |
| | AND MELPHALAN | myeloma. | |
| | 2009 | 2021 Richardson PG, Ha . . . Isatuximab for | |
| | US20110293606 | relapsed/refractory multiple myeloma: | |
| | Lejeune, Pascale; . . . | review of key subgroup analyses from the | |
| | ANTITUMOR | Phase III ICARIA-MM study. | |
| | COMBINATIONS | 2021 Wang A, Song Z, Z . . . Evaluation of | |
| | CONTAINING | Preclinical Activity of Isatuximab in | |
| | ANTIBODIES | Patients with Acute Lymphoblastic | |
| | RECOGNIZING | Leukemia. | |
| | SPECIFICALLY CD38 | 2021 Frampton JE Isatuximab: A Review of | |
| | AND VINCRISTINE | Its Use in Multiple Myeloma. | |
| | 2009 | 2021 Delgado J, Zienow . . . The European | |
| | US20110305690 | Medicines Agency Review of Isatuximab in | |
| | Lejeune, Pascale; . . . | Combination with Pomalidomide and | |
| | ANTITUMOR | Dexamethasone for the Treatment of | |
| | COMBINATIONS | Adult Patients with Relapsed and | |
| | CONTAINING | Refractory Multiple Myeloma. | |
| | ANTIBODIES | 2021 Koiwai K, El-Chei . . . PK/PD Modeling | |
| | RECOGNIZING | Analysis for Dosing Regimen Selection of | |
| | SPECIFICALLY CD38 | Isatuximab as Single Agent and in | |
| | AND | Combination Therapy in Patients with | |
| | CYCLOPHOSPHAMIDE | Multiple Myeloma. | |
| | 2009 | 2021 Moreau P, Dimopou . . . Isatuximab, | |
| | US20120093806 | carfilzomib, and dexamethasone in | |
| | Lejeune, Pascale; . . . | relapsed multiple myeloma (IKEMA): a | |
| | ANTITUMOR | multicentre, open-label, randomised phase | |
| | COMBINATIONS | 3 trial. | |
| | CONTAINING | 2021 Usmani SZ, Karane . . . Final results of a | |
| | ANTIBODIES | phase 1b study of isatuximab short- | |
| | RECOGNIZING | duration fixed-volume infusion | |
| | SPECIFICALLY CD38 | combination therapy for | |
| | AND CYTARABINE | relapsed/refractory multiple myeloma. | |
| | 2009 U.S. Pat. No. 8,633,301 | 2021 Piggin A, Prince HM An evaluation of | |
| | Lejeune, Pascale; . . . | isatuximab, pomalidomide and | |
| | Antitumor | dexamethasone for adult patients with | |
| | combinations | relapsed and refractory multiple myeloma. | |
| | containing antibodies | 2021 Bringhen S, Pour . . . Isatuximab plus | |
| | recognizing | pomalidomide and dexamethasone in | |
| | specifically CD38 and | patients with relapsed/refractory multiple | |
| | vincristine | myeloma according to prior lines of | |
| | 2009 U.S. Pat. No. 9,259,406 | treatment and refractory status: ICARIA- | |
| | Lejeune, Pascale; . . . | MM subgroup analysis. | |
| | Antitumor | 2021 Martin TG, Shah N . . . Phase 1b trial of | |
| | combinations | isatuximab, an anti-CD38 monoclonal | |
| | containing antibodies | antibody, in combination with carfilzomib | |
| | recognizing | as treatment of relapsed/refractory | |
| | specifically CD38 and | multiple myeloma. | |
| | melphalan | 2020 Richardson PG, Be . . . Isatuximab for | |
| | 2007 | the treatment of relapsed/refractory | |
| | US20090304710 | multiple myeloma. | |
| | Park, Peter U.; B . . . | 2020 Fau JB, El-Cheikh . . . Drug-Disease | |
| | NOVEL ANTI-CD38 | Interaction and Time-Dependent | |
| | ANTIBODIES FOR THE | Population Pharmacokinetics of Isatuximab | |
| | TREATMENT OF | in Relapsed/Refractory Multiple Myeloma | |
| | CANCER | Patients. | |
| | 2007 WO2008047242 | 2020 Sunami K, Suzuki . . . Isatuximab | |
| | PARK, Peter U.; B . . . | monotherapy in relapsed/refractory | |
| | NOVEL ANTI-CD38 | multiple myeloma: A Japanese, | |
| | ANTIBODIES FOR THE | multicenter, Phase 1/2, safety and efficacy | |
| | TREATMENT OF | study. | |
| | CANCER | 2020 Jiao Y, Yi M, Xu . . . CD38: Targeted | |
| | 2007 U.S. Pat. No. 8,153,765 | therapy in multiple myeloma and | |
| | Park, Peter U.; B . . . | therapeutic potential for solid cancers. | |
| | Anti-CD38 antibodies | 2020 Schjesvold FH, Ri . . . Isatuximab plus | |
| | for the treatment of | pomalidomide and dexamethasone in | |
| | cancer | elderly patients with relapsed/refractory | |
| | | multiple myeloma: ICARIA-MM subgroup | |
| | | analysis. | |
| | | 2020 Mikhael J, Richte . . . A dose-finding | |
| | | Phase 2 study of single agent isatuximab | |
| | | (anti-CD38 mAb) in relapsed/refractory | |
| | | multiple myeloma. | |
| | | 2020 Dhillon S Isatuximab: First Approval. | |
| | | 2020 Franssen LE, Steg . . . Resistance | |
| | | Mechanisms Towards CD38-Directed | |
| | | Antibody Therapy in Multiple Myeloma. | |
| | | 2019 Moreau P, Dimopou . . . Isatuximab | |
| | | plus carfilzomib/dexamethasone versus | |
| | | carfilzomib/dexamethasone in patients | |
| | | with relapsed/refractory multiple | |
| | | myeloma: IKEMA Phase III study design. | |
| | | 2019 Martin TG, Corzo . . . Therapeutic | |
| | | Opportunities with Pharmacological | |
| | | Inhibition of CD38 with Isatuximab. | |
| | | 2019 Atanackovic D, Yo . . . In vivo | |
| | | vaccination effect in multiple myeloma | |
| | | patients treated with the monoclonal | |
| | | antibody isatuximab. | |
| | | 2019 Martin T, Strickl . . . Phase I trial of | |
| | | isatuximab monotherapy in the treatment | |
| | | of refractory multiple myeloma. | |
| | | 2018 van de Donk NWCJ | |
| | | Immunomodulatory effects of CD38- | |
| | | targeting antibodies. | |
| | | 2018 Kriegsmann K, Dit . . . Quantification of | |
| | | Number of CD38 Sites on Bone Marrow | |
| | | Plasma Cells in Patients with Light Chain | |
| | | Amyloidosis and Smoldering Multiple | |
| | | Myeloma. | |
| | | 2018 Frerichs KA, Nagy . . . CD38-targeting | |
| | | antibodies in multiple myeloma: | |
| | | mechanisms of action and clinical | |
| | | experience. | |
| | | 2017 Richardson PG, At . . . Isatuximab plus | |
| | | pomalidomide/dexamethasone versus | |
| | | pomalidomide/dexamethasone in | |
| | | relapsed/refractory multiple myeloma: | |
| | | ICARIA Phase III study design. | |
| | | 2017 van de Donk NWCJ, . . . CD38 | |
| | | antibodies in multiple myeloma: back to | |
| | | the future. | |
| | | 2017 Martin T, Baz R, . . . A Phase 1b study | |
| | | of isatuximab plus lenalidomide and | |
| | | dexamethasone for relapsed/refractory | |
| | | multiple myeloma. | |
| | | 2017 Feng X, Zhang L, . . . Targeting CD38 | |
| | | suppresses induction and function of T | |
| | | regulatory cells to mitigate | |
| | | immunosuppression in multiple myeloma. | |
| | | 2015 Broijl A, Sonneve . . . An update in | |
| | | treatment options for multiple myeloma in | |
| | | nontransplant eligible patients. | |
| | | 2014 Deckert J, Wetzel . . . SAR650984, a | |
| | | novel humanized CD38-targeting antibody, | |
| | | demonstrates potent anti-tumor activity in | |
| | | models of multiple myeloma and other | |
| | | CD38+ hematologic malignancies. | |
| | | AACR 2013 SAR650984: Characterization of | |
| | | a potent phase I humanized anti-CD38 | |
| | | antibody for the treatment of multiple | |
| | | myeloma and other hematologic | |
| | | malignancies Marie-Cécile Wetz . . . | |
| | | AACR 2013 SAR650984, an anti-CD38 | |
| | | antibody, shows anti-tumor activity in a | |
| | | preclinical model of multiple myeloma | |
| | | Byron Hann, Ti Ca . . . | |
| | | AACR 2015 Expression levels of CD38 and | |
| | | the complement inhibitors CD46, CD55 | |
| | | and CD59 control the ability of anti-CD38 | |
| | | antibodies to trigger complement | |
| | | dependent lysis of multiple myeloma cells | |
| | | Zhili Song, Guang . . . | |
| | | ASCO 2014 A phase I trial of SAR650984, a | |
| | | CD38 monoclonal antibody, in relapsed or | |
| | | refractory multiple myeloma Thomas G. | |
| | | Martin, . . . | |
| | | ASCO 2014 A phase Ib dose escalation trial | |
| | | of SAR650984 (Anti-CD-38 mAb) in | |
| | | combination with lenalidomide and | |
| | | dexamethasone in relapsed/refractory | |
| | | multiple myeloma Thomas G. Martin, . . . | |
| | | ASCO 2016 Updated data from a phase II | |
| | | dose finding trial of single agent | |
| | | isatuximab (SAR650984, anti-CD38 mAb) in | |
| | | relapsed/refractory multiple myeloma | |
| | | (RRMM) Joshua Ryan Richt . . . | |
| | | ASH 2014 SAR 650984, a Therapeutic Anti- | |
| | | CD38 Monoclonal Antibody, Blocks CD38- | |
| | | CD31 Interaction in Multiple Myeloma | |
| | | Gang An, MD, PhD, . . . | |
| | | ASH 2015 A Dose Finding Phase II Trial of | |
| | | Isatuximab (SAR650984, Anti-CD38 mAb) | |
| | | As a Single Agent in Relapsed/Refractory | |
| | | Multiple Myeloma Thomas Martin, MD . . . | |
| | | ASH 2017 Pre-Clinical Efficacy of the Anti- | |
| | | CD38 Monoclonal Antibody (mAb) | |
| | | Isatuximab in Acute Myeloid Leukemia | |
| | | (AML) Tomas Jelinek,,, . . . | |
| Jiangsu | 2020 WO2021115404 | | Jiangsu |
| Hengrui | ZHAO, Xinyan, DEN . . . | | Hengrui |
| patent anti- | ANTI-CD38 | | |
| CD38 | ANTIBODY AND USE | | |
| | THEREOF | | |
| | 2019 WO2020052546 | | |
| | YE, Xin, SUN, Le, . . . | | |
| | ANTI-CD38 | | |
| | ANTIBODY, ANTIGEN- | | |
| | BINDING FRAGMENT | | |
| | THEREOF, AND | | |
| | PHARMACEUTICAL | | |
| | USE | | |
| | 2019 | | |
| | US20220275100 Ye, | | |
| | Xin; Sun, Le; . . . ANTI- | | |
| | CD38 ANTIBODY, | | |
| | ANTIGEN-BINDING | | |
| | FRAGMENT | | |
| | THEREOF, AND | | |
| | PHARMACEUTICAL | | |
| | USE | | |
| Jiangsu Kanion | 2021 WO2021259227 | | Jiangsu Kanion |
| patent anti- | XIAO, Wei, ZHAO, . . . | | |
| CD38 | ANTI-CD38 | | |
| | ANTIBODY AND USE | | |
| | THEREOF | | |
| Jiangsu | | | Jiangsu |
| Simcere | | | Simcere |
| patent anti- | | | |
| CD38 | | | |
| Kleo patent | 2020 WO2021003050 | | Biohaven |
| anti-CD38 | RASTELLI, Luca, W . . . | | Kleo |
| | CD38-BINDING | | |
| | AGENTS AND USES | | |
| | THEREOF | | |
| | 2020 | | |
| | US20230028880 | | |
| | RASTELLI, Luca; W . . . | | |
| | CD38-BINDING | | |
| | AGENTS AND USES | | |
| | THEREOF | | |
| Lentigen | 2021 | | Lentigen |
| patent anti- | US20210353675 | | |
| CD38 CAR | Schneider, Dina; . . . | | |
| | Compositions and | | |
| | Methods for Treating | | |
| | Cancer with Anti- | | |
| | CD38 | | |
| | Immunotherapy | | |
| | 2019 | | |
| | US20200197440 | | |
| | Schneider, Dina; . . . | | |
| | Compositions and | | |
| | Methods for Treating | | |
| | Cancer with Anti- | | |
| | CD38 | | |
| | Immunotherapy | | |
| | 2019 U.S. Pat. No. 11,045,497 | | |
| | Schneider, Dina ( . . . | | |
| | Compositions and | | |
| | methods for treating | | |
| | cancer with anti- | | |
| | CD38 | | |
| | immunotherapy | | |
| | 2019 U.S. Pat. No. 11,103,533 | | |
| | Schneider, Dina ( . . . | | |
| | Compositions and | | |
| | methods for treating | | |
| | cancer with anti- | | |
| | CD38 | | |
| | immunotherapy | | |
| Ligand patent | 2020 WO2021003074 | | Ligand |
| anti-CD38 | IZQUIERDO, Shelle . . . | | |
| | ANTI-CD38 | | |
| | ANTIBODY AND | | |
| | METHODS OF USE | | |
| | THEREOF | | |
| | 2020 | | |
| | US20220306762 | | |
| | Izquierdo, Shelle . . . | | |
| | ANTI-CD38 | | |
| | ANTIBODY AND | | |
| | METHODS OF USE | | |
| | THEREOF | | |
| mezagitamab | 2020 WO2020250033 | 2022 NCT05174221 Phase 1 A Study of | Takeda |
| | PALUMBO, | Mezagitamab in Adults With Primary | |
| | Antonio, . . . | Immunoglobulin A Nephropathy Receiving | |
| | COMBINATION | Stable Background Therapy | |
| | THERAPIES USING | 2021 NCT04776018 Phase 1/Phase 2 A | |
| | CD-38 ANTIBODIES | Study Of TAK-981 Given With Monoclonal | |
| | 2020 | Antibodies In Adults With Relapsed or | |
| | US20220241413 | Refractory Multiple Myeloma | |
| | Palumbo, Antonio; . . . | 2019 NCT03984097 Phase 1 A Study to | |
| | COMBINATION | Evaluate Subcutaneous TAK-079 Added to | |
| | THERAPIES USING | Standard of Care Regimens in Participants | |
| | CD-38 ANTIBODIES | With Newly Diagnosed Multiple Myeloma | |
| | 2019 | (NDMM) | |
| | US20200040105 | 2018 NCT03724916 Phase 1 A Study to | |
| | Elias, Kathleen A . . . | Evaluate the Safety, Pharmacokinetics | |
| | ANTI-CD38 | (PK), and Pharmacodynamics (PD) of TAK- | |
| | ANTIBODIES | 079 in Combination With Standard | |
| | 2019 U.S. Pat. No. 11,434,304 | Background Therapy in Participants With | |
| | Elias, Kathleen A . . . | Moderate to Severe Systemic Lupus | |
| | Anti-CD38 antibodies | Erythematosus (SLE) | |
| | 2019 | 2018 NCT03439280 Phase 1/Phase 2 A | |
| | US20200031951 | Study to Investigate the Safety, | |
| | Elias, Kathleen A . . . | Tolerability, Efficacy, Pharmacokinetics, | |
| | CONJUGATED ANTI- | and Immunogenicity of TAK-079 | |
| | CD38 ANTIBODIES | Administered Subcutaneously as a Single | |
| | 2019 WO2019186273 | Agent in Participants With | |
| | FEDYK, Eric, HANL . . . | Relapsed/Refractory (r/r) Multiple | |
| | SUBCUTANEOUS | Myeloma (MM) | |
| | DOSING OF ANTI- | 2014 NCT02219256 Phase 1 A Phase 1 | |
| | CD38 ANTIBODIES | Study to Assess the Safety, Tolerability, | |
| | 2019 | and Pharmacokinetics of TAK-079 in | |
| | US20210047427 | Healthy Participants | |
| | Fedyk, Eric; Hanl . . . | 2020 Jiao Y, Yi M, Xu . . . CD38: Targeted | |
| | SUBCUTANEOUS | therapy in multiple myeloma and | |
| | DOSING OF ANTI- | therapeutic potential for solid cancers. | |
| | CD38 ANTIBODIES | 2020 Fedyk ER, Zhao L, . . . Safety, | |
| | 2019 WO2019140410 | Tolerability, Pharmacokinetics, and | |
| | DAHL, Martin, FED . . . | Pharmacodynamics of the Anti-CD38 | |
| | SUBCUTANEOUS | Cytolytic Antibody TAK-079 in Healthy | |
| | DOSING OF ANTI- | Subjects. | |
| | CD38 ANTIBODIES | | |
| | 2019 | | |
| | US20210388103 | | |
| | Dahl, Martin; Fed . . . | | |
| | SUBCUTANEOUS | | |
| | ADMINISTRATION OF | | |
| | ANTI-CD38 | | |
| | ANTIBODIES | | |
| | 2017 | | |
| | US20180066069 | | |
| | Elias, Kathleen A . . . | | |
| | ANTI-CD38 | | |
| | ANTIBODIES | | |
| | 2017 U.S. Pat. No. 10,494,444 | | |
| | Elias, Kathleen A . . . | | |
| | Anti-CD38 antibodies | | |
| | 2017 WO2018013917 | | |
| | SMITHSON, | | |
| | Glennda . . . METHODS | | |
| | AND MATERIALS FOR | | |
| | ASSESSING | | |
| | RESPONSE TO | | |
| | PLASMABLAST- AND | | |
| | PLASMA CELL- | | |
| | DEPLETING | | |
| | THERAPIES | | |
| | 2017 | | |
| | US20180016349 | | |
| | ELIAS, Kathleen A . . . | | |
| | CONJUGATED ANTI- | | |
| | CD38 ANTIBODIES | | |
| | 2017 U.S. Pat. No. 10,336,833 | | |
| | Elias, Kathleen A . . . | | |
| | Conjugated anti- | | |
| | CD38 antibodies | | |
| | 2015 | | |
| | US20150291702 | | |
| | ELIAS, Kathleen A . . . | | |
| | CONJUGATED ANTI- | | |
| | CD38 ANTIBODIES | | |
| | 2015 U.S. Pat. No. 9,676,869 | | |
| | Elias, Kathleen A . . . | | |
| | Conjugated anti- | | |
| | CD38 antibodies | | |
| | 2014 | | |
| | US20150203587 | | |
| | Elias, Katheen An . . . | | |
| | ANTI-CD38 | | |
| | ANTIBODIES | | |
| | 2014 U.S. Pat. No. 9,790,285 | | |
| | Elias, Kathleen A . . . | | |
| | Anti-CD38 antibodies | | |
| | 2012 | | |
| | US20130171154 | | |
| | Elias, Kathleen A . . . | | |
| | ANTI-CD38 | | |
| | ANTIBODIES | | |
| | 2012 U.S. Pat. No. 8,926,969 | | |
| | Elias, Kathleen A . . . | | |
| | Anti-CD38 antibodies | | |
| | 2011 WO2012092616 | | |
| | ELIAS, Kathleen, . . . | | |
| | CONJUGATED ANTI- | | |
| | CD38 ANTIBODIES | | |
| | 2011 WO2012092612 | | |
| | ELIAS, Kathleen A . . . | | |
| | ANTI-CD38 | | |
| | ANTIBODIES | | |
| | 2011 U.S. Pat. No. 8,362,211 | | |
| | Elias, Kathleen A . . . | | |
| | Anti-CD38 antibodies | | |
| | 2011 | | |
| | US20140155584 | | |
| | Elias, Kathleen A . . . | | |
| | CONJUGATED ANTI- | | |
| | CD38 ANTIBODIES | | |
| | 2011 U.S. Pat. No. 9,102,744 | | |
| | Elias, Kathleen A . . . | | |
| | Conjugated anti- | | |
| | CD38 antibodies | | |
| Millennium | 2021 | | Millennium |
| patent anti- | US20210369748 | | |
| CD38 | Labotka, Richard; . . . | | |
| | COMBINATION | | |
| | THERAPY FOR | | |
| | CANCER TREATMENT | | |
| | 2021 WO2021231877 | | |
| | HUSZAR, Dennis, N . . . | | |
| | ADMINISTRATION OF | | |
| | SUMO-ACTIVATING | | |
| | ENZYME INHIBITOR | | |
| | AND ANTI-CD38 | | |
| | ANTIBODIES | | |
| | 2021 | | |
| | US20210137955 | | |
| | Labotka, Richard; . . . | | |
| | COMBINATION | | |
| | THERAPY FOR | | |
| | CANCER TREATMENT | | |
| | 2020 WO2020154540 | | |
| | WILLERT, Erin, EL . . . | | |
| | ANTI-CD38 | | |
| | ANTIBODIES | | |
| Momenta | 2020 WO2021055876 | | Momenta |
| patent anti- | ORTIZ, Daniel, CH . . . | | |
| CD38 | COMPOSITIONS AND | | |
| | METHODS RELATED | | |
| | TO ENGINEERED Fc- | | |
| | ANTIGEN BINDING | | |
| | DOMAIN | | |
| | CONSTRUCTS | | |
| | TARGETED TO CD38 | | |
| | 2019 | | |
| | US20210269546 | | |
| | Manning, Anthony; . . . | | |
| | COMPOSITIONS AND | | |
| | METHODS RELATED | | |
| | TO ENGINEERED Fc- | | |
| | ANTIGEN BINDING | | |
| | DOMAIN | | |
| | CONSTRUCTS | | |
| | TARGETED TO CD38 | | |
| OKT10-B10 | | 2020 NCT04579523 Phase 1 ²¹¹At-OKT10- | FHCRC |
| | | B10 and Fludarabine Alone or in | |
| | | Combination With Cyclophosphamide and | |
| | | Low-Dose TBI Before Donor Stem Cell | |
| | | Transplant for the Treatment of Newly | |
| | | Diagnosed, Recurrent, or Refractory High- | |
| | | Risk Multiple Myeloma | |
| | | 2020 NCT04466475 Phase 1 | |
| | | Radioimmunotherapy (211At-OKT10-B10) | |
| | | and Chemotherapy (Melphalan) Before | |
| | | Stem Cell Transplantation for the | |
| | | Treatment of Multiple Myeloma | |
| PeptiDream | 2020 WO2021002265 | | PeptiDream |
| patent anti- | REID CRAWFORD | | |
| CD38 | Pat . . . CD38-BINDING | | |
| | AGENTS AND USES | | |
| | THEREOF | | |
| SAR442085 | | 2019 NCT04000282 Phase 1 First-in-human | Sanofi |
| | | Single Agent Study of SAR442085 in | |
| | | Relapsed or Refractory Multiple Myeloma | |
| | | 2022 Kassem S, Diallo . . . SAR442085, a | |
| | | novel anti-CD38 antibody with enhanced | |
| | | antitumor activity against multiple | |
| | | myeloma. | |
| SG301 | 2019 WO2019154421 | 2021 NCT04684108 Phase 1 SG301 Safety | Hangzhou |
| | LV, Ming, DING, X . . . | Study in Subjects With Relapsed or | Sumgen |
| | CD38 PROTEIN | Refractory Multiple Myeloma and Other | Biotech |
| | ANTIBODY AND | Hematological Malignancies | |
| | APPLICATION | 2019 Yu T, Qiao C, Lv . . . Novel anti-CD38 | |
| | THEREOF | humanized mAb SG003 possessed | |
| | 2019 | enhanced cytotoxicity in lymphoma than | |
| | US20210024645 LV, | Daratumumab via antibody-dependent | |
| | Ming; DING, X . . . CD38 | cell-mediated cytotoxicity. | |
| | PROTEIN ANTIBODY | | |
| | AND APPLICATION | | |
| | THEREOF | | |
| Shanghai Mab | 2021 WO2022095698 | | Shanghai Mab |
| Geek patent | ZHU, Lingqiao, DA . . . | | Geek |
| anti-CD38 | ANTI-HUMAN CD38 | | |
| | ANTIBODY, | | |
| | PREPARATION | | |
| | METHOD THEREFOR | | |
| | AND USE THEREOF | | |
| Shanghai | 2020 WO2021052465 | | Shanghai |
| Puremab | REN, Hongyuan, GA . . . | | Puremab |
| patent anti- | ANTI-HUMAN CD38 | | |
| CD38 | ANTIBODY AND | | |
| | APPLICATION | | |
| | THEREOF | | |
| Sorrento anti- | 2020 | | Sorrento |
| CD38 CAR | US20200399393 Ji, | | |
| | Henry Hongjun . . . | | |
| | Dimeric Antigen | | |
| | Receptors | | |
| STI-6129 | 2021 | 2023 NCT05584709 Phase 1 Study to | Sorrento |
| | US20220118105 Zhu, | Access Anti-CD38 Anibody Drug in Patients | |
| | Tong; Khasan . . . CD38 | With Advanced Solid Tumors | |
| | Antibody Drug | 2023 NCT05692908 Phase 1 An Open-Label | |
| | Conjugate | Study of the Safety of an Anti-CD38 | |
| | 2020 | Antibody Drug Conjugate (STI-6129) in | |
| | US20210130484 | Patients With AL Amyloidosis | |
| | Swanson, Barbara . . . | 2023 NCT05519527 Phase 1 A Phase 1, | |
| | Variant Antibody | Open-Label, Dose-Escalation Study of | |
| | That Binds Cd38 | Safety And Efficacy of An Anti-CD38 | |
| | 2020 | Antibody Drug Conjugate (STI-6129) In | |
| | US20200399395 | Patients With Relapsed Or Refractory T- | |
| | Zhou, Heyue; Gray . . . | Acute Lymphoblastic Leukemia/Lymphoma | |
| | Antibody | (T-ALL) Or Acute Myeloid Leukemia (AML) | |
| | Therapeutics that | 2022 NCT05565807 Phase 1/Phase 2 | |
| | Bind CD38 | Safety and Efficacy Study of An Anti-CD38 | |
| | 2020 WO2020205499 | Antibody Drug Conjugate in Relapsed or | |
| | CAO, Xia, ZHOU, H . . . | Refractory Multiple Myeloma | |
| | ENGINEERED | 2022 NCT05308225 Phase 1 Study to | |
| | VARIANT ANTIBODIES | Assess Anti-CD38 Antibody Drug Conjugate | |
| | THAT BIND CD38 | in Relapsed or Refractory Multiple | |
| | 2020 | Myeloma | |
| | US20220144966 Cao, | 2020 NCT04316442 Phase 1 Anti-CD38- | |
| | Xia; Zhou, H . . . | Duostatin 5.2 ADC for AL Amyloidosis | |
| | Engineered Variant | | |
| | Antibodies that Bind | | |
| | CD38 | | |
| | 2019 WO2019245616 | | |
| | SWANSON, | | |
| | Barbara, . . . VARIANT | | |
| | ANTIBODY THAT | | |
| | BINDS CD38 | | |
| | 2018 | | |
| | US20190135937 | | |
| | Zhang, Yanliang; . . . | | |
| | CD-38 Directed | | |
| | Chimeric Antigen | | |
| | Receptor Constructs | | |
| | 2018 WO2019087151 | | |
| | ZHANG, Yanliang, . . . | | |
| | CD38-DIRECTED | | |
| | CHIMERIC ANTIGEN | | |
| | RECEPTOR | | |
| | CONSTRUCTS | | |
| | 2018 | | |
| | US20190048092 | | |
| | Zhou, Heyue; Gray . . . | | |
| | ANTIBODY | | |
| | THERAPEUTICS THAT | | |
| | BIND CD38 | | |
| | 2018 U.S. Pat. No. 10,800,852 | | |
| | Zhou, Heyue (San . . . | | |
| | Antibody | | |
| | therapeutics that | | |
| | bind CD38 | | |
| | 2018 | | |
| | US20180360985 Zhu, | | |
| | Tong; Khasan . . . CD38 | | |
| | Antibody Drug | | |
| | Conjugate | | |
| | 2018 WO2018235024 | | |
| | ZHU, Tong, KHASAN . . . | | |
| | CD38 ANTIBODY | | |
| | DRUG CONJUGATE | | |
| | 2018 U.S. Pat. No. 11,191,845 | | |
| | Zhu, Tong (San Di . . . | | |
| | CD38 antibody drug | | |
| | conjugate | | |
| | 2016 | | |
| | US20160297888 | | |
| | Zhou, Heyue; Gray . . . | | |
| | ANTIBODY | | |
| | THERAPEUTICS THAT | | |
| | BIND CD38 | | |
| | 2016 WO2016164656 | | |
| | GRAY, John, Dixon . . . | | |
| | ANTIBODY | | |
| | THERAPEUTICS THAT | | |
| | BIND CD38 | | |
| | 2016 WO2016164669 | | |
| | ZHOU, Heyue, | | |
| | GRAY . . . ANTIBODY | | |
| | THERAPEUTICS THAT | | |
| | BIND CD38 | | |
| | 2016 | | |
| | US20170291959 | | |
| | Gray, John Dixon; . . . | | |
| | Antibody | | |
| | Therapeutics That | | |
| | Bind CD38 | | |
| | 2016 U.S. Pat. No. 9,951,144 | | |
| | Gray, John Dixon; . . . | | |
| | Antibody | | |
| | therapeutics that | | |
| | bind CD38 | | |
| | 2016 U.S. Pat. No. 10,059,774 | | |
| | Zhou, Heyue (San . . . | | |
| | Antibody | | |
| | therapeutics that | | |
| | bind CD38 | | |
| Sunshine | 2021 WO2022033535 | | Sunshine |
| Guojian | GUO, Wei, ZHAO, J . . . | | Guojian |
| patent anti- | ANTIBODY BINDING | | Pharma |
| CD38 | TO HUMAN CD38, | | |
| | PREPARATION | | |
| | METHOD THEREOF, | | |
| | AND USE THEREOF | | |
| Suzhou | 2018 WO2020056790 | | Acroimmune |
| Stainwei Bio | HU, Hongqun, | | Suzhou |
| patent anti- | SONG . . . | | Stainwei Bio |
| CD38 | MONOCLONAL | | |
| | ANTIBODY | | |
| | SPECIFICALLY | | |
| | BINDING HUMAN | | |
| | AND MONKEY CD38 | | |
| | ANTIGEN, AND | | |
| | PREPARATION | | |
| | METHOD THEREFOR | | |
| | AND APPLICATION | | |
| | THEREOF | | |
| | 2018 | | |
| | US20210324102 HU, | | |
| | Hongqun; SONG . . . | | |
| | MONOCLONAL | | |
| | ANTIBODY | | |
| | SPECIFICALLY | | |
| | BINDING HUMAN | | |
| | AND MONKEY CD38 | | |
| | ANTIGENS, | | |
| | PREPARATION | | |
| | METHOD AND USE | | |
| | THEREOF | | |
| Teva patent | 2018 | 2016 Pogue SL, Taura T . . . Targeting | Teva |
| anti-CD38 | US20180305460 | Attenuated Interferon-α to Myeloma Cells | |
| Interferon | Clarke, Adam; Pol . . . | with a CD38 Antibody Induces Potent | |
| fusion | ANTI-CD38 | Tumor Regression with Reduced Off-Target | |
| | ANTIBODIES AND | Activity. | |
| | FUSIONS TO | | |
| | ATTENUATED | | |
| | INTERFERON ALPHA- | | |
| | 2B | | |
| | 2018 U.S. Pat. No. 11,117,975 | | |
| | Clarke, Adam (Mac . . . | | |
| | Anti-CD38 antibodies | | |
| | and fusions to | | |
| | attenuated | | |
| | interferon alpha-2B | | |
| | 2017 | | |
| | US20170233449 | | |
| | Wilson; Jr., Davi . . . | | |
| | FUSIONS OF | | |
| | ANTIBODIES TO CD38 | | |
| | AND ATTENUATED | | |
| | INTERFERON ALPHA | | |
| | 2017 U.S. Pat. No. 10,981,986 | | |
| | Wilson; Jr., Davi . . . | | |
| | Fusions of antibodies | | |
| | to CD38 and | | |
| | attenuated | | |
| | interferon alpha | | |
| | 2017 U.S. Pat. No. 10,232,041 | | |
| | Pogue, Sarah L. ( . . . | | |
| | Combination of | | |
| | lenalidomide and | | |
| | polypeptide | | |
| | construct, and uses | | |
| | thereof | | |
| | 2016 | | |
| | US20160367695 | | |
| | Wilson, David S.; . . . | | |
| | POLYPEPTIDE | | |
| | CONSTRUCTS AND | | |
| | USES THEREOF | | |
| | 2015 | | |
| | US20160068612 | | |
| | Clarke, Adam; Pol . . . | | |
| | ANTI-CD38 | | |
| | ANTIBODIES AND | | |
| | FUSIONS TO | | |
| | ATTENUATED | | |
| | INTERFERON ALPHA- | | |
| | 2B | | |
| | 2015 U.S. Pat. No. 9,963,515 | | |
| | Clarke, Adam; Pol . . . | | |
| | Anti-CD38 antibodies | | |
| | and fusions to | | |
| | attenuated | | |
| | interferon alpha-2B | | |
| | 2015 | | |
| | US20150313965 | | |
| | Pogue, Sarah L.; . . . | | |
| | Combination of | | |
| | Lenalidomide and | | |
| | Polypeptide | | |
| | Construct, and Uses | | |
| | Thereof | | |
| | 2015 U.S. Pat. No. 9,636,334 | | |
| | Pogue, Sarah L.; . . . | | |
| | Combination of | | |
| | lenalidomide and | | |
| | polypeptide | | |
| | construct, and uses | | |
| | thereof | | |
| | 2014 U.S. Pat. No. 9,611,322 | | |
| | Wilson; Jr., Davi . . . | | |
| | Fusions of antibodies | | |
| | to CD38 and | | |
| | attenuated | | |
| | interferon alpha | | |
| | 2013 WO2014178820 | | |
| | CLARKE, Adam, POL . . . | | |
| | ANTI-CD38 | | |
| | ANTIBODIES AND | | |
| | FUSIONS TO | | |
| | ATTENUATED | | |
| | INTERFERON ALPHA- | | |
| | 2B | | |
| TNB-738 | 2022 WO2022271987 | 2022 NCT05215912 Phase 1 A Single and | Ancora |
| | UGAMRAJ, Harshad | Multiple Dosing Study Targeting | TeneoBio |
| | ANTI-CD38 | Biparatopic Antibody CD38 in Healthy | |
| | ANTIBODIES AND | Volunteers | |
| | EPITOPES OF SAME | | |
| | 2020 WO2021127489 | | |
| | DALVI, Pranjali, . . . | | |
| | HEAVY CHAIN | | |
| | ANTIBODIES BINDING | | |
| | TO CD38 | | |
| | 2020 | | |
| | US20220372162 | | |
| | Dalvi, III, Pranj . . . | | |
| | PCT/US2020/066088 | | |
| | 2019 WO2020087065 | | |
| | VAN SCHOOTEN, | | |
| | Wim . . . HEAVY CHAIN | | |
| | ANTIBODIES BINDING | | |
| | TO CD38 | | |
| | 2019 | | |
| | US20210388106 van | | |
| | Schooten, Wim . . . | | |
| | HEAVY CHAIN | | |
| | ANTIBODIES BINDING | | |
| | TO CD38 | | |
| | 2018 | | |
| | US20200207867 | | |
| | Clarke, Starlynn; . . . | | |
| | HEAVY CHAIN | | |
| | ANTIBODIES BINDING | | |
| | TO ECTOENZYMES | | |
| U. California | 2019 | | U. California |
| patent anti- | US20200046832 | | |
| CD38 | HANN, Byron C.; T . . . | | |
| | COMPOSITIONS | | |
| | COMPRISING ANTI- | | |
| | CD38 ANTIBODIES | | |
| | AND LENALIDOMIDE | | |
| | 2015 | | |
| | US20170224817 | | |
| | Venstrom, Jeffrey . . . | | |
| | METHODS FOR | | |
| | TREATING MULTIPLE | | |
| | MYELOMA | | |
| U. Liege anti- | 2021 WO2022242892 | 2021 Duray E, Lejeune . . . A non- | U. Liege |
| CD38 | CAERS, Jo ANTI-CD38 | internalised CD38-binding radiolabelled | |
| | SINGLE-DOMAIN | single-domain antibody fragment to | |
| | ANTIBODIES IN | monitor and treat multiple myeloma. | |
| | DISEASE | | |
| | MONITORING AND | | |
| | TREATMENT | | |
| | 2021 WO2021229104 | | |
| | CAERS, Jo, DURAY, . . . | | |
| | ANTI-CD38 SINGLE- | | |
| | DOMAIN ANTIBODIES | | |
| | IN DISEASE | | |
| | MONITORING AND | | |
| | TREATMENT | | |
| UMC Utrecht | 2017 WO2018002181 | | UMC Utrecht |
| patent anti- | OTTEN, Henny G. | | |
| CD38 | TREATMENT OF IgE- | | |
| | MEDIATED DISEASES | | |
| | WITH ANTIBODIES | | |
| | THAT SPECIFICALLY | | |
| | BIND CD38 | | |
| | 2017 | | |
| | US20190233533 | | |
| | Otten, Henny G.; | | |
| | Treatment Of IgE- | | |
| | Mediated Diseases | | |
| | With Antibodies That | | |
| | Specifically Bind | | |
| | CD38 | | |
| Y-Mabs patent | 2021 WO2021254574 | | Y-mAbs |
| anti-CD38 | MAHIUDDIN, | | |
| | Ahmed, . . . CD38 | | |
| | ANTIBODIES FOR THE | | |
| | TREATMENT OF | | |
| | HUMAN DISEASES | | |
| Yeda patent | 2018 WO2019111249 | | Yeda R&D |
| anti-CD38 CAR | ESHHAR, Zelig, WA . . . | | |
| | T-CELLS COMPRISING | | |
| | ANTI-CD38 AND | | |
| | ANTI-CD138 | | |
| | CHIMERIC ANTIGEN | | |
| | RECEPTORS AND | | |
| | USES THEREOF | | |
| AMG 424 | 2019 WO2020018556 | 2018 NCT03445663 Phase 1 Study | Amgen |
| | YAGO, Marc | Evaluating AMG 424 in Subjects With | Xencor |
| | Anthon . . . METHOD | Multiple Myeloma | |
| | OF TREATING | 2019 Zuch de Zafra CL, . . . Targeting | |
| | MULTIPLE MYELOMA | Multiple Myeloma with AMG 424, a Novel | |
| | 2019 WO2019157340 | Anti-CD38/CD3 Bispecific T Cell-Recruiting | |
| | CHRISTIAN, Twinkl . . . | Antibody Optimized for Cytotoxicity and | |
| | LOW PH | Cytokine Release. | |
| | PHARMACEUTICAL | | |
| | ANTIBODY | | |
| | FORMULATION | | |
| | 2019 | | |
| | US20210047407 | | |
| | Christian, Twinkl . . . | | |
| | LOW PH | | |
| | PHARMACEUTICAL | | |
| | ANTIBODY | | |
| | FORMULATION | | |
| | 2016 WO2017091656 | | |
| | STEVENS, Jennitte . . . | | |
| | HETERODIMERIC | | |
| | ANTIBODIES THAT | | |
| | CD3 AND CD38 | | |
| BioGraph 55 | 2022 | | BioGraph 55 |
| patent anti- | US20220185907 | | |
| CD19 / CD38 | PRESTA, Leonard; . . . | | |
| | ANTI-CD19/ANTI- | | |
| | CD38 COMMON | | |
| | LIGHT CHAIN | | |
| | BISPECIFIC | | |
| | ANTIBODIES | | |
| | 2022 | | |
| | US20220185906 | | |
| | PRESTA, Leonard; . . . | | |
| | ANTI-CD19/ANTI- | | |
| | CD38 COMMON | | |
| | LIGHT CHAIN | | |
| | BISPECIFIC | | |
| | ANTIBODIES | | |
| | 2022 | | |
| | US20220185908 | | |
| | PRESTA, Leonard; . . . | | |
| | METHODS OF USE OF | | |
| | ANTI-CD19/ANTI- | | |
| | CD38 COMMON | | |
| | LIGHT CHAIN | | |
| | BISPECIFIC | | |
| | ANTIBODIES | | |
| | 2022 | | |
| | US20220363774 | | |
| | PRESTA, Leonard; . . . | | |
| | ANTI-CD19/ANTI- | | |
| | CD38 COMMON | | |
| | LIGHT CHAIN | | |
| | BISPECIFIC | | |
| | ANTIBODIES | | |
| | 2021 U.S. Pat. No. 11,299,551 | | |
| | Presta, Leonard ( . . . | | |
| | Composite binding | | |
| | molecules targeting | | |
| | immunosuppressive | | |
| | B cells | | |
| | 2021 WO2021173844 | | |
| | PRESTA, Leonard, . . . | | |
| | C19 C38 BISPECIFIC | | |
| | ANTIBODIES | | |
| BMX-101 | 2017 WO2017162890 | | Biomunex |
| | ZHUKOVSKY, | | |
| | Eugene . . . BINDING | | |
| | MOLECULES TO CD38 | | |
| | AND PD-L1 | | |
| City of Hope | 2021 WO2022094147 | | City of Hope |
| patent anti- | MARCUCCI, Guido, . . . | | |
| CD38 / CD3 | BISPECIFIC ANTI- | | |
| | CD38-CD3 BINDERS | | |
| Cytovia patent | 2022 WO2022216723 | | Cytovia |
| anti-NKP46 / | KADOUCHE, Jean | | |
| CD38 | BISPECIFIC | | |
| | ANTIBODIES | | |
| | TARGETING NKP46 | | |
| | AND CD38 AND | | |
| | METHODS OF USE | | |
| | THEREOF | | |
| GBR 1342 | 2020 WO2020182984 | | Glenmark / |
| | DUBEY, Sachin, BI . . . | | Ichnos |
| | NON-CONSENSUS | | |
| | GLYCOSYLATION OF | | |
| | BISPECIFIC | | |
| | ANTIBODIES | | |
| | 2018 | | |
| | US20190135918 | | |
| | OLLIER, Romain; H . . . | | |
| | CD3/CD38 T CELL | | |
| | RETARGETING | | |
| | HETERO-DIMERIC | | |
| | IMMUNOGLOBULINS | | |
| | AND METHODS OF | | |
| | THEIR PRODUCTION | | |
| | 2015 WO2016071355 | | |
| | OLLIER, Romain, . . . | | |
| | CD3/CD38 T CELL | | |
| | RETARGETING | | |
| | HETERO-DIMERIC | | |
| | IMMUNOGLOBULINS | | |
| | AND METHODS OF | | |
| | THEIR PRODUCTION | | |
| Ichnos patent | 2021 | | Glenmark / |
| anti-CD47 / | US20220089767 | | Ichnos |
| CD38 | Bouchez, Laure; P . . . | | |
| | CD47-CD38 | | |
| | BISPECIFIC | | |
| | ANTIBODIES | | |
| | 2021 WO2022058539 | | |
| | BOUCHEZ, Laure, P . . . | | |
| | CD47-CD38 | | |
| | BISPECIFIC | | |
| | ANTIBODIES | | |
| IGM-2644 | | | IGM Bio |
| INSERM | 2020 WO2021009263 | | INSERM |
| patent anti- | BENSUSSAN, | | |
| CD3 / CD38 | Armand . . . | | |
| | ANTIBODIES HAVING | | |
| | SPECIFICITY FOR | | |
| | CD38 AND USES | | |
| | THEREOF | | |
| | 2020 | | |
| | US20220251234 | | |
| | Bensussan, Armand . . . | | |
| | ANTIBODIES HAVING | | |
| | SPECIFICITY FOR | | |
| | CD38 AND USES | | |
| | THEREOF | | |
| Regeneron | 2021 | | Regeneron |
| patent anti- | US20220089766 | | |
| CD28 / CD38 | DiLillo, David; H . . . | | |
| | ANTIGEN-BINDING | | |
| | MOLECULES THAT | | |
| | BIND CD38 AND/OR | | |
| | CD28, AND USES | | |
| | THEREOF | | |
| | 2021 WO2022061098 | | |
| | DILILLO, David, H . . . | | |
| | ANTIGEN-BINDING | | |
| | MOLECULES THAT | | |
| | BIND CD38 AND/OR | | |
| | CD28, AND USES | | |
| | THEREOF | | |
| SG2501 | 2022 NCT05293912 | | Hangzhou |
| | Phase 1 SG2501 | | Sumgen |
| | Safety Study in | | Biotech |
| | Subjects With | | |
| | Relapsed or | | |
| | Refractory | | |
| | Hematological | | |
| | Malignancies and | | |
| | Lymphoma. | | |
| Sorrento | 2020 WO2021003189 | | Sorrento |
| patent anti- | HE, Xiao, ZHANG, . . . | | |
| CD38 / CD3 | HETERODIMERIC | | |
| | ANTIBODIES THAT | | |
| | BIND TO CD38 AND | | |
| | CD3 | | |
| | 2020 | | |
| | US20220363758 He, | | |
| | Xiao; Zhang, . . . | | |
| | Heterodimeric | | |
| | Antibodies That Bind | | |
| | to CD38 and CD3 | | |
| Virtuoso | 2021 WO2021229306 | | Virtuoso |
| patent anti- | CHEN, Xiaocheng, . . . | | |
| CD38 / BCMA | MULTISPECIFIC | | |
| | ANTIBODIES | | |
| | TARGETING CD38 | | |
| | AND BCMA AND | | |
| | USES THEREOF | | |
| Virtuoso | 2021 WO2021231975 | 2022 NCT05698888 Phase 1 Study of | U. California |
| patent anti- | CHEN, Xiaocheng | VP301 in Patients With Multiple Myeloma, | Virtuoso |
| CD38 / ICAM-1 | HUMANIZED CD38 | Lymphoma, or Solid Tumors | |
| | AND ICAM1 | | |
| | ANTIBODIES AND | | |
| | USES THEREOF | | |
| | 2019 WO2020102777 | | |
| | LIU, Bin, CHEN, X . . . | | |
| | CD38 AND ICAM1 | | |
| | ANTIBODIES AND | | |
| | USES THEREOF | | |
| | 2019 | | |
| | US20220002432 LIU, | | |
| | Bin; CHEN, X . . . CD38 | | |
| | AND ICAM1 | | |
| | ANTIBODIES AND | | |
| | USES THEREOF | | |
| XmAb968 | 2022 | | Xencor |
| | US20220403049 | | |
| | Bernett, Matthew; . . . | | |
| | HETERODIMERIC | | |
| | ANTIBODIES THAT | | |
| | BIND CD3 AND CD38 | | |
| | 2020 | | |
| | US20210309762 | | |
| | Bernett, Matthew; . . . | | |
| | Bispecific Antibodies | | |
| | That Bind to CD38 | | |
| | and CD3 | | |
| | 2019 | | |
| | US20200199251 | | |
| | Bernett, Matthew . . . | | |
| | HETERODIMERIC | | |
| | ANTIBODIES THAT | | |
| | BIND CD3 AND CD38 | | |
| | 2019 U.S. Pat. No. 11,352,442 | | |
| | Bernett, Matthew . . . | | |
| | Heterodimeric | | |
| | antibodies that bind | | |
| | CD3 and CD38 | | |
| | 2017 | | |
| | US20180094079 | | |
| | Bernett, Matthew . . . | | |
| | Bispecific Antibodies | | |
| | That Bind to CD38 | | |
| | and CD3 | | |
| | 2017 U.S. Pat. No. 10,858,451 | | |
| | Bernett, Matthew . . . | | |
| | Bispecific antibodies | | |
| | that bind to CD38 | | |
| | and CD3 | | |
| | 2016 | | |
| | US20180305465 | | |
| | Stevens, Jennitte . . . | | |
| | HETERODIMERIC | | |
| | ANTIBODIES THAT | | |
| | BIND CD3 AND CD38 | | |
| | 2015 WO2016086196 | | |
| | MOORE, Gregory, . . . | | |
| | HETERODIMERIC | | |
| | ANTIBODIES THAT | | |
| | BIND CD3 AND CD38 | | |
| | 2015 | | |
| | US20160215063 | | |
| | Bernett, Matthew . . . | | |
| | HETERODIMERIC | | |
| | ANTIBODIES THAT | | |
| | BIND CD3 AND CD38 | | |
| | 2015 U.S. Pat. No. 10,526,417 | | |
| | Bernett, Matthew . . . | | |
| | Heterodimeric | | |
| | antibodies that bind | | |
| | CD3 and CD38 | | |
| | 2015 WO2015149077 | | |
| | BERNETT, | | |
| | Matthew, . . . | | |
| | BISPECIFIC | | |
| | ANTIBODIES THAT | | |
| | BIND TO CD38 AND | | |
| | CD3 | | |
| | 2015 | | |
| | US20150307629 | | |
| | Bernett, Matthew; . . . | | |
| | Bispecific antibodies | | |
| | that bind to CD38 | | |
| | and CD3 | | |
| | 2015 U.S. Pat. No. 9,822,186 | | |
| | Bernett, Matthew; . . . | | |
| | Bispecific antibodies | | |
| | that bind to CD38 | | |
| | and CD3 | | |
| Y150 | | 2021 NCT05011097 Phase 1 A Phase I | Wuhan YZY |
| | | Clinical Trial of Y150 in the Treatment of | Biopharma |
| | | Relapsed or Refractory Multiple Myeloma | |
| SAR442257 | 2020 WO2020210392 | 2020 NCT04401020 Phase 1 First-in-human | Sanofi |
| | YANG, Zhi-Yong, B . . . | Single Agent Study of SAR442257 in RRMM | |
| | TRISPECIFIC BINDING | and RR-NHL | |
| | PROTEINS, | | |
| | METHODS, AND USES | | |
| | THEREOF | | |
| | 2020 | | |
| | US20200399369 | | |
| | ASOKAN, | | |
| | Mangaiark . . . | | |
| | TRISPECIFIC BINDING | | |
| | PROTEINS, | | |
| | METHODS, AND USES | | |
| | THEREOF | | |
| | 2019 WO2020076853 | | |
| | WU, Lan, XU, Ling . . . | | |
| | TRISPECIFIC ANTI- | | |
| | CD38, ANTI-CD28, | | |
| | AND ANTI-CD3 | | |
| | BINDING PROTEINS | | |
| | AND METHODS OF | | |
| | USE FOR TREATING | | |
| | VIRAL INFECTION | | |
| | 2019 | | |
| | US20200140552 WU, | | |
| | Lan; XU, Ling . . . | | |
| | TRISPECIFIC ANTI- | | |
| | CD38, ANTI-CD28, | | |
| | AND ANTI-CD3 | | |
| | BINDING PROTEINS | | |
| | AND METHODS OF | | |
| | USE FOR TREATING | | |
| | VIRAL INFECTION | | |
| | 2019 U.S. Pat. No. 11,530,268 | | |
| | Wu, Lan; Xu, Ling . . . | | |
| | Trispecific anti-CD38, | | |
| | anti-CD28, and anti- | | |
| | CD3 binding proteins | | |
| | and methods of use | | |
| | for treating viral | | |
| | infection | | |

g. CD70 CAR

In some embodiments, the CAR is a CD70 CAR (“CD70-CAR”), and in these embodiments, the polycistronic vector comprises an expression cassette that contains a nucleotide sequence encoding a CD70 CAR. CD70 is highly expressed on AML blasts and leukemia stem cells. In some embodiments, the CD70 CAR may comprise a signal peptide, an extracellular binding domain that specifically binds CD70, a hinge domain, a transmembrane domain, an intracellular costimulatory domain, and/or an intracellular signaling domain in tandem.

In some embodiments, the signal peptide of the CD70 CAR comprises a CD8α signal peptide. In some embodiments, the CD8α signal peptide comprises or consists of an amino acid sequence set forth in SEQ ID NO:6 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:6. In some embodiments, the signal peptide comprises an IgK signal peptide. In some embodiments, the IgK signal peptide comprises or consists of an amino acid sequence set forth in SEQ ID NO:7 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:7. In some embodiments, the signal peptide comprises a GMCSFR-α or CSF2RA signal peptide. In some embodiments, the GMCSFR-α or CSF2RA signal peptide comprises or consists of an amino acid sequence set forth in SEQ ID NO:8 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:8.

In some embodiments, the extracellular binding domain of the CD70 CAR is specific to CD70, for example, human CD70. The extracellular binding domain of the GPRC5D CAR can be codon-optimized for expression in a host cell or to have variant sequences to increase functions of the extracellular binding domain. In some embodiments, the extracellular binding domain comprises an immunogenically active portion of an immunoglobulin molecule, for example, an scFv.

In some embodiments, the extracellular binding domain of the CD70 CAR is derived from an antibody specific to CD70, including, for example, any of the antibodies or CARs disclosed in Table 24, the references cited in which are incorporated by reference in their entireties herein. In any of these embodiments, the extracellular binding domain of the CD70 CAR can comprise or consist of the V_{H}, the V_{L}, and/or one or more CDRs of any of the antibodies described herein, including in Table 24.

In some embodiments, the extracellular binding domain of the CD70 CAR comprises an scFv derived from the any of the antibodies or CARs disclosed in Table 24, optionally comprising the heavy chain variable region (V_{H}) and the light chain variable region (V_{L}) of one of the antibodies or CARs, connected by a linker. In some embodiments, the linker is a 3×G₄S linker. In other embodiments, the Whitlow linker may be used instead. In some embodiments, the CD70-specific scFv comprises or consists of the scFv of an antibody or CAR disclosed in Table 24, or an amino acid sequence that is at least 80% identical (e.g., at least 80%, at least 85%, at least 90%, at least 95%, at least 96%, at least 97%, at least 98%, at least 99%, or 100% identical) to the amino acid sequence of the scFv of an antibody or CAR disclosed in Table 24. In some embodiments, the CD70-specific scFv may comprise one or more CDRs having amino acid sequences of the CDRs of an antibody or CAR disclosed in Table 24. In some embodiments, the CD70-specific scFv may comprise a heavy chain with one or more CDRs having amino acid sequences of the CDRs of an antibody or CAR disclosed in Table 24. In some embodiments, the CD70-specific scFv may comprise a light chain with one or more CDRs having amino acid sequences of the CDRs of an antibody or CAR disclosed in Table 24. In any of these embodiments, the CD70-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the CD70 CAR comprises or consists of the one or more CDRs as described herein, including in Table 24.

TABLE 24
Exemplary CD70 antigen binding domains

| Name | Antigen | Company | Reference |
|---|---|---|---|
| | CD70 | Ambrx | U.S. Pat. No. 10,208,123 |
| AMG 172 | CD70 | Amgen, ImmunoGen | U.S. Pat. No. 8,987,422 |
| CTX130 | CD70 | Crispr Therapeutics | |
| | CD70 | Crispr Therapeutics | WO2021245603 |
| cusatuzumab | CD70 | Argenx, Janssen-Cilag | U.S. Pat. No. 9,765,148 |
| | CD70 | ImaginAb | WO2014158821 |
| | CD70 | Jiangsu Hengrui | WO2022002019 |
| | CD70 | Kite | U.S. Pat. No. 11,046,775 |
| | CD70 | Mass. General Hosp. | WO2021055437 |
| MDX-1203 | CD70 | Medarex | US20090028872 |
| MDX-1411 | CD70 | Medarex | US20100150950 |
| | CD70 | Ambrx Merck (MSD) | WO2013/192360A1 |
| | CD70 | NIH | U.S. Pat. No. 10,689,456 |
| | CD70 | Osaka U. | |
| | CD70 | Pfizer | WO2019152742 |
| SGN-CD70A | CD70 | Seattle Genetics | WO2021138264 |
| vorsetuzumab | CD70 | Seattle Genetics | US20210002380 |
| vorsetuzumab | CD70 | Seattle Genetics | US20110150908 |
| mafodotin | | | |
| | CD70, CD3 | Amgen | U.S. Pat. No. 10,851,170 |
| | CD70 | Crispr Therapeutics | WO2019097305A2 |
| | CD70, CD3 | Pfizer | US20190233529 |

In some embodiments, the hinge domain of the CD70 CAR comprises a CD8α hinge domain, for example, a human CD8α hinge domain. In some embodiments, the CD8α hinge domain comprises or consists of an amino acid sequence set forth in SEQ ID NO:9 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:9. In some embodiments, the hinge domain comprises a CD28 hinge domain, for example, a human CD28 hinge domain. In some embodiments, the CD28 hinge domain comprises or consists of an amino acid sequence set forth in SEQ ID NO:10 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:10. In some embodiments, the hinge domain comprises an IgG4 hinge domain, for example, a human IgG4 hinge domain. In some embodiments, the IgG4 hinge domain comprises or consists of an amino acid sequence set forth in SEQ ID NO:11 or SEQ ID NO:12, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:11 or SEQ ID NO:12. In some embodiments, the hinge domain comprises a IgG4 hinge-Ch2-Ch3 domain, for example, a human IgG4 hinge-Ch2-Ch3 domain. In some embodiments, the IgG4 hinge-Ch2-Ch3 domain comprises or consists of an amino acid sequence set forth in SEQ ID NO:13 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:13.

In some embodiments, the transmembrane domain of the CD70 CAR comprises a CD8α transmembrane domain, for example, a human CD8α transmembrane domain. In some embodiments, the CD8α transmembrane domain comprises or consists of an amino acid sequence set forth in SEQ ID NO:14 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:14. In some embodiments, the transmembrane domain comprises a CD28 transmembrane domain, for example, a human CD28 transmembrane domain. In some embodiments, the CD28 transmembrane domain comprises or consists of an amino acid sequence set forth in SEQ ID NO:15 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:15.

In some embodiments, the intracellular costimulatory domain of the CD70 CAR comprises a 4-1BB costimulatory domain, for example, a human 4-1BB costimulatory domain. In some embodiments, the 4-1BB costimulatory domain comprises or consists of an amino acid sequence set forth in SEQ ID NO:16 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:16. In some embodiments, the intracellular costimulatory domain comprises a CD28 costimulatory domain, for example, a human CD28 costimulatory domain. In some embodiments, the CD28 costimulatory domain comprises or consists of an amino acid sequence set forth in SEQ ID NO:17 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:17.

In some embodiments, the intracellular signaling domain of the CD70 CAR comprises a CD3 zeta (ζ) signaling domain, for example, a human CD3ζ signaling domain. In some embodiments, the CD3ζ signaling domain comprises or consists of an amino acid sequence set forth in SEQ ID NO:18 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:18.

In some embodiments, the polycistronic vector comprises an expression cassette that contains a nucleotide sequence encoding a CD70 CAR, including, for example, a CD70 CAR comprising the CD70-specific scFv having sequences of an antibody or CAR disclosed in Table 24, the CD8α hinge domain of SEQ ID NO:9, the CD8α transmembrane domain of SEQ ID NO:14, the 4-1BB costimulatory domain of SEQ ID NO:16, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD70 CAR, including, for example, a CD70 CAR comprising the CD70-specific scFv having sequences of an antibody or CAR disclosed in Table 24, the CD28 hinge domain of SEQ ID NO:10, the CD8α transmembrane domain of SEQ ID NO:14, the 4-1BB costimulatory domain of SEQ ID NO:16, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD70 CAR, including, for example, a CD70 CAR comprising the CD70-specific scFv having sequences of an antibody or CAR disclosed in Table 24, the IgG4 hinge domain of SEQ ID NO:11 134 or SEQ ID NO:12, the CD8α transmembrane domain of SEQ ID NO:14, the 4-1BB costimulatory domain of SEQ ID NO:16, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD70 CAR, including, for example, a CD70 CAR comprising the CD70-specific scFv having sequences of an antibody or CAR disclosed in Table 24, the CD8α hinge domain of SEQ ID NO:9, the CD28 transmembrane domain of SEQ ID NO:15, the 4-1BB costimulatory domain of SEQ ID NO:16, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD70 CAR, including, for example, a CD70 CAR comprising the CD70-specific scFv having sequences of an antibody or CAR disclosed in Table 24, the CD28 hinge domain of SEQ ID NO:10, the CD28 transmembrane domain of SEQ ID NO:15, the 4-1BB costimulatory domain of SEQ ID NO:16, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD70 CAR, including, for example, a CD70 CAR comprising the CD70-specific scFv having sequences of an antibody or CAR disclosed in Table 24, the IgG4 hinge domain of SEQ ID NO:11 or SEQ ID NO:12, the CD28 transmembrane domain of SEQ ID NO:15, the 4-1BB costimulatory domain of SEQ ID NO:16, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, a polynucleotide provided herein comprises a nucleotide sequence encoding a CD70 CAR, a variable domain of a CD70 CAR, or a set of CDRs (HCDR 1, 2, and 3 and LCDR 1, 2, and 3) that encodes a CD70 CAR as set forth in TABLE 25 below or a variable domain of a CD70 CAR or a set of CDRs (HCDR 1, 2, and 3 and LCDR 1, 2, and 3) therefrom. In some embodiments, a polynucleotide provided herein comprises a nucleotide sequence encoding a CD70 CAR, a variable domain of a CD70 CAR, or a set of CDRs (HCDR 1, 2, and 3 and LCDR 1, 2, and 3) that having at least 80% (e.g., at least 80%, at least 85%, at least 90%, at least 95%, at least 96%, at least 97%, at least 98%, at least 99%, or 100% identical) to a CD70 CAR as set forth in TABLE 25 below or a variable domain of a CD70 CAR or a set of CDRs (HCDR 1, 2, and 3 and LCDR 1, 2, and 3) therefrom, respectively.

TABLE 25
Exemplary CD70 antigen binding domains

| Antibody Name | Patents | Publications | Company |
|---|---|---|---|
| Ambrx patent | 2018 US20190338039 | | Ambrx |
| anti-CD70 | Barnett, Richard . . . | | |
| | ANTI-CD70 ANTIBODY | | |
| | DRUG CONJUGATES | | |
| | 2018 U.S. Pat. No. 11,459,392 | | |
| | Barnett, Richard S Anti- | | |
| | CD70 antibody drug | | |
| | conjugates | | |
| | 2016 WO2017062271 | | |
| | BRANDISH, Philip, . . . | | |
| | ANTIBODY DRUG | | |
| | CONJUGATE FOR ANTI- | | |
| | INFLAMMATORY | | |
| | APPLICATIONS | | |
| | 2013 WO2013192360 | | |
| | BARNETT, Richard, . . . | | |
| | ANTI-CD70 ANTIBODY | | |
| | DRUG CONJUGATES | | |
| | 2013 US20150141624 | | |
| | Barnett, Richard . . . | | |
| | Anti-CD70 Antibody | | |
| | Drug Conjugates | | |
| | 2013 U.S. Pat. No. 10,208,123 | | |
| | Barnett, Richard . . . | | |
| | Anti-CD70 antibody | | |
| | drug conjugates | | |
| AMG 172 | 2017 US20170267775 | 2011 NCT01497821 | Amgen ImmunoGen |
| | Delaney, John M.; . . . | Phase 1 AMG 172 First | |
| | CD27L Antigen Binding | in Human Study in | |
| | Proteins | Patients With Kidney | |
| | 2017 U.S. Pat. No. 10,266,604 | Cancer | |
| | Delaney, John M. . . . | 2019 Massard C, Soria . . . | |
| | CD27L antigen binding | First-in-human study | |
| | proteins | to assess safety, | |
| | 2015 US20150218284 | tolerability, | |
| | DELANEY, John M.; . . . | pharmacokinetics, and | |
| | CD27L ANTIGEN | pharmacodynamics of | |
| | BINDING PROTEINS | the anti-CD27L | |
| | 2015 U.S. Pat. No. 9,574,008 | antibody-drug | |
| | Delaney, John M.; . . . | conjugate AMG 172 in | |
| | CD27L antigen binding | patients with | |
| | proteins | relapsed/refractory | |
| | 2012 US20130078237 | renal cell carcinoma. | |
| | DELANEY, John M.; . . . | | |
| | CD27L ANTIGEN | | |
| | BINDING PROTEINS | | |
| | 2012 WO2013043933 | | |
| | DELANEY, John M., . . . | | |
| | CD27L ANTIGEN | | |
| | BINDING PROTEINS | | |
| | 2012 U.S. Pat. No. 8,987,422 | | |
| | Delaney, John M.; . . . | | |
| | CD27L antigen binding | | |
| | proteins | | |
| Crispr Thera patent | 2022 US20220378829 | | Crispr Thera |
| anti-CD70 | TERRETT, Jonathan . . . | | |
| | GENETICALLY | | |
| | ENGINEERED IMMUNE | | |
| | CELLS TARGETING | | |
| | CD70 FOR USE IN | | |
| | TREATING SOLID | | |
| | TUMORS | | |
| | 2021 US20220041754 | | |
| | KUMAR, Lalit; DEQ . . . | | |
| | ANTI-IDIOTYPE | | |
| | ANTIBODIES | | |
| | TARGETING ANTI-CD70 | | |
| | CHIMERIC ANTIGEN | | |
| | RECEPTOR | | |
| | 2021 WO2022029629 | | |
| | KUMAR, Lalit, DEQ . . . | | |
| | ANTI-IDIOTYPE | | |
| | ANTIBODIES | | |
| | TARGETING ANTI-CD70 | | |
| | CHIMERIC ANTIGEN | | |
| | RECEPTOR | | |
| | 2021 WO2021245603 | | |
| | SAGERT, Jason, PH . . . | | |
| | ANTI-CD70 | | |
| | ANTIBODIES AND USES | | |
| | THEREOF | | |
| cusatuzumab | 2021 WO2022043538 | 2019 NCT04150887 | argenx Janssen-Cilag |
| | DE HAARD, Johanne . . . | Phase 1 Cusatuzumab | |
| | METHOD OF | in Combination With | |
| | TREATMENT OF | Background Therapy | |
| | PATIENTS HAVING | for the Treatment of | |
| | REDUCED SENSITIVITY | Participants With Acute | |
| | TO A BCL-2 INHIBITOR | Myeloid Leukemia | |
| | 2019 US20200222532 | 2019 NCT04023526 | |
| | DE HAARD, Johanne . . . | Phase 2 A Study of | |
| | CD70 COMBINATION | Cusatuzumab Plus | |
| | THERAPY | Azacitidine in | |
| | 2019 US20190270823 | Participants With | |
| | Silence, Karen; U . . . | Newly Diagnosed Acute | |
| | ANTIBODIES TO CD70 | Myeloid Leukemia Who | |
| | 2019 U.S. Pat. No. 11,434,298 | Are Not Candidates for | |
| | Silence, Karen (O . . . | Intensive | |
| | Antibodies to CD70 | Chemotherapy | |
| | 2019 WO2019141732 | 2016 NCT03030612 | |
| | VAN ROMPAEY, Luc | Phase 1/Phase 2 ARGX- | |
| | CD70 COMBINATION | 110 With AZA in AML | |
| | THERAPY | or High Risk MDS | |
| | 2019 US20190241668 | 2015 NCT02759250 | |
| | VAN ROMPAEY, Luc; | Phase 1 A Study of | |
| | CD70 COMBINATION | ARGX-110 in Patients | |
| | THERAPY | With Nasopharyngeal | |
| | 2019 U.S. Pat. No. 11,530,271 Van | Carcinoma (NPC) | |
| | Rompaey, Luc; . . . CD70 | 2013 NCT01813539 | |
| | combination therapy | Phase 1 Phase 1b Study | |
| | 2018 WO2018229303 | of ARGX-110 in | |
| | LEUPIN, Nicolas, . . . USE | Patients With | |
| | OF ANTI CD70 | Advanced Malignancies | |
| | ANTIBODY ARGX-110 | 2022 Ikezoe T, Usuki | |
| | TO TREAT ACUTE | K . . . Cusatuzumab Plus | |
| | MYELOID LEUKAEMIA | Azacitidine in Japanese | |
| | 2015 US20150266963 | Patients With Newly | |
| | SILENCE, Karen; U . . . | Diagnosed Acute | |
| | ANTIBODIES TO CD70 | Myeloid Leukemia | |
| | 2015 US20170369581 | Ineligible for Intensive | |
| | SILENCE, Karen; U . . . | Treatment. | |
| | ANTIBODIES TO CD70 | 2021 Leupin N, | |
| | 2015 U.S. Pat. No. 11,072,665 | Zinzani . . . Cusatuzumab | |
| | Silence, Karen (O . . . | for treatment of CD70- | |
| | Antibodies to CD70 | positive relapsed or | |
| | 2014 US20140235843 | refractory cutaneous T- | |
| | SILENCE, Karen; U . . . | cell lymphoma. | |
| | ANTIBODIES TO CD70 | 2021 De Meulenaere A, . . . | |
| | 2014 U.S. Pat. No. 9,765,149 | An open-label, non- | |
| | Silence, Karen; U . . . | randomized, Phase Ib | |
| | Antibodies to CD70 | feasibility study of | |
| | 2013 US20140147450 | cusatuzumab in | |
| | SILENCE, Karen; U . . . | patients with | |
| | ANTIBODIES TO CD70 | nasopharyngeal | |
| | 2013 U.S. Pat. No. 8,834,882 | carcinoma. | |
| | Silence, Karen; U . . . | 2020 The CD70 | |
| | Antibodies to CD70 | Antibody Cusatuzumab | |
| | 2012 WO2012123586 | Shows Promise in | |
| | SILENCE, Karen, U . . . | Acute Myeloid | |
| | ANTIBODIES TO CD70 | Leukemia. | |
| | 2012 US20140141016 | 2017 Aftimos P, Rolfo . . . | |
| | Silence, Karen; U . . . | Phase I Dose- | |
| | ANTIBODIES TO CD70 | Escalation Study of the | |
| | 2012 U.S. Pat. No. 9,765,148 | Anti-CD70 Antibody | |
| | Silence, Karen; U . . . | ARGX-110 in Advanced | |
| | Antibodies to CD70 | Malignancies. | |
| | | 2013 Silence K, Dreier . . . | |
| | | ARGX-110, a highly | |
| | | potent antibody | |
| | | targeting CD70, | |
| | | eliminates tumors via | |
| | | both enhanced ADCC | |
| | | and immune | |
| | | checkpoint blockade. | |
| | | AACR 2013 Pre-clinical | |
| | | characterization of | |
| | | ARGX-110: A | |
| | | neutralizing, | |
| | | humanized monoclonal | |
| | | antibody to the human | |
| | | CD70 antigen with | |
| | | enhanced ADCC | |
| | | properties Karen | |
| | | Silence, Ha . . . | |
| | | ASCO 2014 A phase I, | |
| | | first-in-human study of | |
| | | ARGX-110, a | |
| | | monoclonal antibody | |
| | | targeting CD70, a | |
| | | receptor involved in | |
| | | immune escape and | |
| | | tumor growth in | |
| | | patients with solid and | |
| | | hematologic | |
| | | malignancies | |
| ImaginAb patent | 2014 WO2014158821 | | ImaginAb |
| anti-CD70 | HO, David, T., OL . . . | | |
| | ANTIGEN BINDING | | |
| | CONSTRUCTS TO CD70 | | |
| Jlangsu Hengrui | 2021 WO2022002019 | | Jiangsu Hengrui |
| patent | SUN, Le, YE, Xin, . . . | | |
| anti-CD70 | ANTI-CD70 ANTIBODY | | |
| | AND APPLICATION | | |
| | THEREOF | | |
| Jiangsu Simcere | 2021 WO2022105914 | | Jiangsu Simcere |
| patent | MA, Xiaoli, CAO, . . . | | |
| anti-CD70 | ANTIBODY BINDING TO | | |
| | CD70 AND | | |
| | APPLICATION THEREOF | | |
| Keymed patent | 2021 WO2022143951 | | Keymed |
| anti-CD70 | XU, Gang, CHEN, B . . . | | |
| | DEVELOPMENT AND | | |
| | USE OF FUNCTION- | | |
| | ENHANCED ANTIBODY | | |
| | BLOCKING AGENT | | |
| Kite patent | 2021 US20210277132 | | Kite |
| anti-CD70 | ALVAREZ RODRIGUEZ . . . | | |
| | CD70 Binding | | |
| | Molecules and | | |
| | Methods of Use | | |
| | Thereof | | |
| | 2018 US20180230224 | | |
| | ALVAREZ RODRIGUEZ . . . | | |
| | CD70 BINDING | | |
| | MOLECULES AND | | |
| | METHODS OF USE | | |
| | THEREOF | | |
| | 2018 WO2018152181 | | |
| | ALVAREZ RODRIGUEZ . . . | | |
| | CD70 BINDING | | |
| | MOLECULES AND | | |
| | METHODS OF USE | | |
| | THEREOF | | |
| | 2018 U.S. Pat. No. 11,046,775 | | |
| | Alvarez Rodriguez . . . | | |
| | CD70 binding | | |
| | molecules and | | |
| | methods of use thereof | | |
| Mass. General Hosp. | 2020 WO2021055437 | | Mass. General Hosp. |
| patent anti-CD70 CAR | LEICK, Mark, MAUS . . . | | |
| | CD70 TARGETED | | |
| | CHIMERIC ANTIGEN | | |
| | RECEPTOR (CAR) T | | |
| | CELLS AND USES | | |
| | THEREOF | | |
| MDX-1203 | 2007 WO2008074004 | 2009 NCT00944905 | Medarex |
| | COCCIA, Marco, A . . . | Phase 1 Study of MDX- | |
| | HUMAN ANTIBODIES | 1203 in Subjects With | |
| | THAT BIND CD70 AND | Advanced/Recurrent | |
| | USES THEREOF | Clear Cell Renal Cell | |
| | 2007 US20100150950 | Carcinoma (ccRCC) or | |
| | Coccia, Marco A.; . . . | Relapsed/Refractory B- | |
| | HUMAN ANTIBODIES | Cell Non-Hodgkin's | |
| | THAT BIND CD70 AND | Lymphoma (B-NHL) | |
| | USES THEREOF | | |
| | 2006 WO2007038637 | | |
| | TERRETT, Jonathan . . . | | |
| | HUMAN | | |
| | MONOCLONAL | | |
| | ANTIBODIES TO CD70 | | |
| | 2006 WO2007038658 | | |
| | BOYD, Sharon, Ela . . . | | |
| | ANTIBODY-DRUG | | |
| | CONJUGATES AND | | |
| | METHODS OF USE | | |
| | 2006 U.S. Pat. No. 8,124,738 | | |
| | Terret, Jonathan . . . | | |
| | Human monoclonal | | |
| | antibodies to CD70 | | |
| | 2006 US20090028872 | | |
| | Terret, Jonathan . . . | | |
| | HUMAN | | |
| | MONOCLONAL | | |
| | ANTIBODIES TO CD70 | | |
| MDX-1411 | 2014 US20140323690 | 2009 NCT00730652 | Medarex |
| | Cheng, Heng; Gang . . . | Phase 1 Study of MDX- | |
| | ANTIPROLIFERATIVE | 1411 in Patients With | |
| | COMPOUNDS, | Relapsed/Refractory | |
| | CONJUGATES | Chronic Lymphocytic | |
| | THEREOF, METHODS | Leukemia or Mantle | |
| | THEREFOR, AND USES | Cell Lymphoma | |
| | THEREOF | 2008 NCT00656734 | |
| | 2014 U.S. Pat. No. 9,226,974 | Phase 1 Study of MDX- | |
| | Cheng, Heng; Gang . . . | 1411 Given Every 14 | |
| | Antiproliferative | Days With Pre- | |
| | compounds, | medications to | |
| | conjugates thereof, | Subjects With Clear Cell | |
| | methods therefor, and | Kidney Cancer. | |
| | uses thereof | | |
| | 2007 WO2008074004 | | |
| | COCCIA, Marco, A . . . | | |
| | HUMAN ANTIBODIES | | |
| | THAT BIND CD70 AND | | |
| | USES THEREOF | | |
| | 2007 US20100150950 | | |
| | Coccia, Marco A.; . . . | | |
| | HUMAN ANTIBODIES | | |
| | THAT BIND CD70 AND | | |
| | USES THEREOF | | |
| | 2006 WO2007038637 | | |
| | TERRETT, Jonathan . . . | | |
| | HUMAN | | |
| | MONOCLONAL | | |
| | ANTIBODIES TO CD70 | | |
| | 2006 WO2007038658 | | |
| | BOYD, Sharon, Ela . . . | | |
| | ANTIBODY-DRUG | | |
| | CONJUGATES AND | | |
| | METHODS OF USE | | |
| | 2006 U.S. Pat. No. 8,124,738 | | |
| | Terret, Jonathan . . . | | |
| | Human monoclonal | | |
| | antibodies to CD70 | | |
| | 2006 US20090028872 | | |
| | Terret, Jonathan . . . | | |
| | HUMAN | | |
| | MONOCLONAL | | |
| | ANTIBODIES TO CD70 | | |
| Merck patent | 2016 WO2017062271 | | Ambrx Merck (MSD) |
| anti-CD70 | BRANDISH, Philip, . . . | | |
| | ANTIBODY DRUG | | |
| | CONJUGATE FOR ANTI- | | |
| | INFLAMMATORY | | |
| | APPLICATIONS | | |
| Nanjing Blue Shield | 2021 WO2022262099 | | Zhejiang Blue Shield |
| patent anti-CD70 | ZHANG, Junfeng ANTI- | | |
| | CD70 INTERNALIZED | | |
| | ANTIBODY, ANTIBODY | | |
| | CONJUGATE AND | | |
| | APPLICATION THEREOF | | |
| | 2021 WO2022262100 | | |
| | ZHANG, Junfeng ANTI- | | |
| | CD70 ANTIBODY | | |
| | HAVING ENHANCED | | |
| | ADCP EFFECT AND | | |
| | APPLICATION THEREOF | | |
| | 2021 WO2022262101 | | |
| | ZHANG, Junfeng ANTI- | | |
| | CD70 ANTIBODY | | |
| | HAVING ENHANCED | | |
| | ADCC EFFECT AND | | |
| | APPLICATION THEREOF | | |
| Nanjing Iaso | 2021 WO2022078344 | | Iaso |
| Bio patent | YANG, Yongkun, HU . . . | | |
| anti-CD70 CAR | ANTIBODY AND | | |
| | CHIMERIC ANTIGEN | | |
| | RECEPTOR (CAR) | | |
| | BINDING TO CD70, | | |
| | AND APPLICATION | | |
| | THEREOF | | |
| NIH patent | 2020 US20200317807 | | NIH |
| anti-CD70 | Wang, Qiong J.; Y . . . | | |
| CAR | ANTI-CD70 CHIMERIC | | |
| | ANTIGEN RECEPTORS | | |
| | 2015 WO2016093878 | | |
| | WANG, Qiong J., . . . | | |
| | ANTI-CD70 CHIMERIC | | |
| | ANTIGEN RECEPTORS | | |
| | 2015 US20180208671 | | |
| | Wang, Qiong J.; Y . . . | | |
| | ANTI-CD70 CHIMERIC | | |
| | ANTIGEN RECEPTORS | | |
| | 2015 U.S. Pat. No. 10,689,456 | | |
| | Wang, Qiong J. (P . . . | | |
| | Anti-CD70 chimeric | | |
| | antigen receptors | | |
| Osaka U. | | 2021 Shiomi M, | Osaka U. |
| anti-CD70 | | Matsuza . . . CD70 | |
| | | antibody-drug | |
| | | conjugate: A potential | |
| | | novel therapeutic | |
| | | agent for ovarian | |
| | | cancer. | |
| Pfizer patent | 2019 WO2019152742 | | Pfizer |
| anti-CD70 | SRIVATSA SRINIVAS . . . | | |
| CAR | CHIMERIC ANTIGEN | | |
| | RECEPTORS | | |
| | TARGETING CD70 | | |
| PRO1160 | 2022 WO2022226317 | | ProfoundBio |
| | ZHAO, Baiteng ANTI- | | |
| | CD70 ANTIBODIES, | | |
| | CONJUGATES THEREOF | | |
| | AND METHODS OF | | |
| | USING THE SAME | | |
| SGN-CD70A | 2022 WO2023278377 | 2014 NCT02216890 | Seattle Genetics |
| | DIOLAITI, Daniel | Phase 1 Safety Study of | |
| | METHODS OF | SGN-CD70A in Cancer | |
| | TREATING CANCER | Patients | |
| | WITH A COMBINATION | 2021 Wu C H, Wang L, | |
| | OF A | Ya . . . Targeting CD70 in | |
| | NONFUCOSYLATED | cutaneous T-cell | |
| | ANTI-CD70 ANTIBODY | lymphoma using an | |
| | AND A CD47 | antibody-drug | |
| | ANTAGONIST | conjugate in patient- | |
| | 2020 WO2021138264 | derived xenograft | |
| | GARDAI, Shyra, HO . . . | models. | |
| | METHODS OF | 2019 Pal S K, Forero- | |
| | TREATING CANCER | To . . . A phase 1 trial of | |
| | WITH | SGN-CD70A in patients | |
| | NONFUCOSYLATED | with CD70-positive, | |
| | ANTI-CD70 | metastatic renal cell | |
| | ANTIBODIES | carcinoma. | |
| | 2014 WO2014165119 | 2018 Phillips T, Barr . . . | |
| | HUI, Li, JIANG, S . . . | A phase 1 trial of SGN- | |
| | CYCLODEXTRIN AND | CD70A in patients with | |
| | ANTIBODY-DRUG | CD70-positive diffuse | |
| | CONJUGATE | large B cell lymphoma | |
| | FORMULATIONS | and mantle cell | |
| | | lymphoma. | |
| | | AACR 2014 SGN- | |
| | | CD70A, a novel and | |
| | | highly potent anti- | |
| | | CD70 ADC, induces | |
| | | double-strand DNA | |
| | | breaks and is active in | |
| | | models of MDR+ renal | |
| | | cell carcinoma (RCC) | |
| | | and non-Hodgkin | |
| | | lymphoma (NHL) | |
| U. Texas patent | 2022 WO2023278520 | | U. Texas |
| anti-CD70 | REZVANI, Katy | | |
| CAR | POLYPEPTIDES | | |
| | TARGETING CD70- | | |
| | POSITIVE CANCERS | | |
| | 2022 WO2022159791 | | |
| | REZVANI, Katy, SH . . . | | |
| | CD27-EXTRACELLULAR | | |
| | DOMAIN CAR TO | | |
| | TARGET CD70-POSITIVE | | |
| | TUMORS | | |
| vorsetuzumab | 2020 US20210002380 | 2009 Law C L, | Seattle Genetics |
| | McDonagh, Charlot . . . | McEarcher . . . Novel | |
| | Humanized Anti-CD70 | antibody-based | |
| | Binding Agents and | therapeutic agents | |
| | Uses Thereof | targeting CD70: a | |
| | 2017 US20170342157 | potential approach for | |
| | McDonagh, Charlot . . . | treating Waldenström's | |
| | Humanized Anti-CD70 | macroglobulinemia. | |
| | Binding Agents and | 2008 McEarchern J A, | |
| | Uses Thereof | Sm . . . Preclinical | |
| | 2016 US20170022282 | characterization of | |
| | McDonagh, Charlot . . . | SGN-70, a humanized | |
| | Humanized Anti-CD70 | antibody directed | |
| | Binding Agents and | against CD70. | |
| | Uses Thereof | 2008 Ho A W, | |
| | 2016 U.S. Pat. No. 9,701,752 | Hatjiharis . . . CD27-CD70 | |
| | McDonagh, Charlot . . . | interactions in the | |
| | Humanized anti-CD70 | pathogenesis of | |
| | binding agents and | Waldenstrom | |
| | uses thereof | macroglobulinemia. | |
| | 2015 US20160003847 | 2006 McEarchern J A, | |
| | Ryan, Maureen; Sm . . . | Of . . . Engineered anti- | |
| | Detection and | CD70 antibody with | |
| | Treatment of | multiple effector | |
| | Pancreatic, Ovarian | functions exhibits in | |
| | and Other CD70 | vitro and in vivo | |
| | Positive Cancers | antitumor activities. | |
| | 2014 WO2014165119 | | |
| | HUI, Li, JIANG, S . . . | | |
| | CYCLODEXTRIN AND | | |
| | ANTIBODY-DRUG | | |
| | CONJUGATE | | |
| | FORMULATIONS | | |
| | 2013 US20140178936 | | |
| | MCDONAGH, | | |
| | CHARLOT . . . Humanized | | |
| | Anti-CD70 Binding | | |
| | Agents and Uses | | |
| | Thereof | | |
| | 2013 U.S. Pat. No. 9,428,585 | | |
| | McDonagh, Charlot . . . | | |
| | Humanized anti-CD70 | | |
| | binding agents and | | |
| | uses thereof | | |
| | 2012 US20120294863 | | |
| | Law, Che-Leung; M . . . | | |
| | Anti-CD70 Antibody | | |
| | and Its Use for the | | |
| | Treatment and | | |
| | Prevention of Cancer | | |
| | and Immune Disorders | | |
| | 2012 US20120251559 | | |
| | Law, Che-Leung; W . . . | | |
| | Treatment of B-Cell | | |
| | Cancers With Anti- | | |
| | CD70 Antibody-Drug | | |
| | Conjugates | | |
| | 2012 US20120288512 | | |
| | Law, Che-Leung; W . . . | | |
| | Anti-CD70 Antibody- | | |
| | Drug Conjugates and | | |
| | Their Use for the | | |
| | Treatment of Cancer | | |
| | and Immune Disorders | | |
| | 2012 U.S. Pat. No. 8,535,678 Law, | | |
| | Cheu-leung; . . . Anti- | | |
| | CD70 antibody-drug | | |
| | conjugates and their | | |
| | use for the treatment | | |
| | of cancer and immune | | |
| | disorders | | |
| | 2011 US20120045436 | | |
| | McDonagh, Charlot . . . | | |
| | Humanized Anti-CD70 | | |
| | Binding Agents and | | |
| | Uses Thereof | | |
| | 2011 U.S. Pat. No. 8,562,987 | | |
| | McDonagh, Charlot . . . | | |
| | Humanized anti-CD70 | | |
| | binding agents and | | |
| | uses thereof | | |
| | 2010 US20110150908 | | |
| | LAW, CHE-LEUNG; M . . . | | |
| | ANTI-CD70 ANTIBODY- | | |
| | DRUG CONJUGATES | | |
| | AND THEIR USE FOR | | |
| | THE TREATMENT AND | | |
| | PREVENTION OF | | |
| | CANCER AND IMMUNE | | |
| | DISORDERS | | |
| | 2010 U.S. Pat. No. 8,663,642 Law, | | |
| | Che-Leung; M . . . Anti- | | |
| | CD70 antibody-drug | | |
| | conjugates and their | | |
| | use for the treatment | | |
| | and prevention of | | |
| | cancer and immune | | |
| | disorders | | |
| | 2009 US20100129362 | | |
| | LAW, CHE-LEUNG; M . . . | | |
| | TREATMENT OF | | |
| | PSORIATIC ARTHRITIS | | |
| | WITH ANTI-CD70 | | |
| | ANTIBODY | | |
| | 2009 US20090232806 | | |
| | Law, Che-Leung; M . . . | | |
| | Anti-CD70 Antibody | | |
| | And Its Use For The | | |
| | Treatment And | | |
| | Prevention Of Cancer | | |
| | And Immune Disorders | | |
| | 2009 U.S. Pat. No. 8,337,838 Law, | | |
| | Che-Leung; M . . . Anti- | | |
| | CD70 antibody and its | | |
| | use for the treatment | | |
| | and prevention of | | |
| | cancer and immune | | |
| | disorders | | |
| | 2008 US20090074772 | | |
| | Law, Che-Leung; M . . . | | |
| | Anti-CD70 Antibody | | |
| | and Its Use for the | | |
| | Treatment of Cancer | | |
| | and Immune Disorders | | |
| | 2008 U.S. Pat. No. 9,051,372 Law, | | |
| | Che-Leung; M . . . Anti- | | |
| | CD70 antibody and its | | |
| | use for the treatment | | |
| | of cancer and immune | | |
| | disorders | | |
| | 2008 U.S. Pat. No. 8,647,624 Law, | | |
| | Che-Leung; M . . . | | |
| | Treatment of immune | | |
| | disorders with anti- | | |
| | CD70 antibody | | |
| | 2007 U.S. Pat. No. 7,641,903 Law, | | |
| | Che-Leung; M . . . Anti- | | |
| | CD70 antibody and its | | |
| | use for the treatment | | |
| | and prevention of | | |
| | cancer and immune | | |
| | disorders | | |
| | 2006 US20090148942 | | |
| | McDonagh, Charlot . . . | | |
| | HUMANIZED ANTI- | | |
| | CD70 BINDING AGENTS | | |
| | AND USES THEREOF | | |
| | 2006 U.S. Pat. No. 8,067,546 | | |
| | McDonagh, Charlot . . . | | |
| | Humanized anti-CD70 | | |
| | binding agents and | | |
| | uses thereof | | |
| | 2005 WO2006044643 | | |
| | LAW, Che-Leung; M . . . | | |
| | ANTI-CD70 ANTIBODY | | |
| | AND ITS USE FOR THE | | |
| | TREATMENT AND | | |
| | PREVENTION OF | | |
| | CANCER AND IMMUNE | | |
| | DISORDERS | | |
| | 2005 U.S. Pat. No. 7,491,390 Law, | | |
| | Che-Leung; M . . . Anti- | | |
| | CD70 antibody and its | | |
| | use for the treatment | | |
| | and prevention of | | |
| | cancer and immune | | |
| | disorders | | |
| | 2004 WO2004073656 | | |
| | LAW, Che-Leung; W . . . | | |
| | ANTI-CD70 ANTIBODY- | | |
| | DRUG CONJUGATES | | |
| | AND THEIR USE FOR | | |
| | THE TREATMENT OF | | |
| | CANCER AND IMMUNE | | |
| | DISORDERS | | |
| vorsetuzumab | 2020 US20210221897 | 2012 NCT01677390 | Seattle Genetics |
| mafodotin | GARDAI, Shyra; HO . . . | Phase 1 A Phase 1b | |
| | METHODS OF | Study of SGN-75 in | |
| | TREATING CANCER | Combination With | |
| | WITH | Everolimus in Patients | |
| | NONFUCOSYLATED | With Renal Cell | |
| | ANTI-CD70 | Carcinoma | |
| | ANTIBODIES | 2009 NCT01015911 | |
| | 2020 US20210002380 | Phase 1 A Phase 1 | |
| | McDonagh, Charlot . . . | Dose-escalation Trial of | |
| | Humanized Anti-CD70 | SGN-75 in CD70- | |
| | Binding Agents and | positive Non-Hodgkin | |
| | Uses Thereof | Lymphoma or Renal | |
| | 2017 US20170232112 | Cell Carcinoma | |
| | Li, Hui; Jiang, S . . . | 2014 Tannir N M, | |
| | CYCLODEXTRIN AND | Forero . . . Phase I dose- | |
| | ANTIBODY-DRUG | escalation study of | |
| | CONJUGATE | SGN-75 in patients with | |
| | FORMULATIONS | CD70-positive | |
| | 2015 WO2015123679 | relapsed/refractory | |
| | DORONINA, Svetlan . . . | non-Hodgkin | |
| | HYDROPHILIC | lymphoma or | |
| | ANTIBODY-DRUG | metastatic renal cell | |
| | CONJUGATES | carcinoma. | |
| | 2014 WO2014165119 | 2010 Ryan M C, Kostner . . . | |
| | HUI, Li, JIANG, S . . . | Targeting pancreatic | |
| | CYCLODEXTRIN AND | and ovarian | |
| | ANTIBODY-DRUG | carcinomas using the | |
| | CONJUGATE | auristatin-based anti- | |
| | FORMULATIONS | CD70 antibody-drug | |
| | 2014 US20160045615 | conjugate SGN-75. | |
| | Li, Hui; Jiang, S . . . | 2009 Oflazoglu E, | |
| | CYCLODEXTRIN AND | Bour ... Blocking of | |
| | ANTIBODY-DRUG | CD27-CD70 pathway by | |
| | CONJUGATE | anti-CD70 antibody | |
| | FORMULATIONS | ameliorates joint | |
| | 2014 U.S. Pat. No. 9,610,361 Li, | disease in murine | |
| | Hui; Jiang, S . . . | collagen-induced | |
| | Cyclodextrin and | arthritis. | |
| | antibody-drug | 2009 Alley S C, Zhang | |
| | conjugate formulations | X . . . The pharmacologic | |
| | 2012 US20120294863 | basis for antibody- | |
| | Law, Che-Leung; M . . . | auristatin conjugate | |
| | Anti-CD70 Antibody | activity. | |
| | and Its Use for the | 2008 Oflazoglu E, | |
| | Treatment and | Ston . . . Potent | |
| | Prevention of Cancer | anticarcinoma activity | |
| | and Immune Disorders | of the humanized anti- | |
| | 2012 US20120251559 | CD70 antibody h1F6 | |
| | Law, Che-Leung; W . . . | conjugated to the | |
| | Treatment of B-Cell | tubulin inhibitor | |
| | Cancers With Anti- | auristatin via an | |
| | CD70 Antibody-Drug | uncleavable linker. | |
| | Conjugates | 2008 McDonagh C F, | |
| | 2012 U.S. Pat. No. 8,609,104 Law, | Kim . . . Engineered anti- | |
| | Che-leung; W . . . | CD70 antibody-drug | |
| | Treatment of B-cell | conjugate with | |
| | cancers with anti-CD70 | increased therapeutic | |
| | antibody-drug | index. | |
| | conjugates | 2006 Law C L, Gordon | |
| | 2012 US20120288512 | K A . . . Lymphocyte | |
| | Law, Che-Leung; W . . . | activation antigen | |
| | Anti-CD70 Antibody- | CD70 expressed by | |
| | Drug Conjugates and | renal cell carcinoma is | |
| | Their Use for the | a potential therapeutic | |
| | Treatment of Cancer | target for anti-CD70 | |
| | and Immune Disorders | antibody-drug | |
| | 2011 US20120045436 | conjugates. | |
| | McDonagh, Charlot . . . | AACR 2013 | |
| | Humanized Anti-CD70 | Development of | |
| | Binding Agents and | pyrrolobenzodiazepine- | |
| | Uses Thereof | based antibody-drug | |
| | 2010 US20110150908 | conjugates for cancer | |
| | LAW, CHE-LEUNG; M . . . | Scott C. Jeffrey | |
| | ANTI-CD70 ANTIBODY- | | |
| | DRUG CONJUGATES | | |
| | AND THEIR USE FOR | | |
| | THE TREATMENT AND | | |
| | PREVENTION OF | | |
| | CANCER AND IMMUNE | | |
| | DISORDERS | | |
| | 2009 US20100158910 | | |
| | Law, Che-Leung; W . . . | | |
| | TREATMENT OF RENAL | | |
| | CELL CARCINOMA | | |
| | WITH ANTI-CD70 | | |
| | ANTIBODY-DRUG | | |
| | CONJUGATES | | |
| | 2009 US20100150925 | | |
| | Law, Che-Leung; W . . . | | |
| | TREATMENT OF B-CELL | | |
| | CANCERS WITH ANTI- | | |
| | CD70 ANTIBODY-DRUG | | |
| | CONJUGATES | | |
| | 2009 U.S. Pat. No. 9,345,785 Law, | | |
| | Che-Leung; W . . . | | |
| | Treatment of renal cell | | |
| | carcinoma with anti- | | |
| | CD70 antibody-drug | | |
| | conjugates | | |
| | 2009 US20100129362 | | |
| | LAW, CHE-LEUNG; M . . . | | |
| | TREATMENT OF | | |
| | PSORIATIC ARTHRITIS | | |
| | WITH ANTI-CD70 | | |
| | ANTIBODY | | |
| | 2009 US20100183636 | | |
| | Law, Che-leung; W . . . | | |
| | ANTI-CD70 ANTIBODY- | | |
| | DRUG CONJUGATES | | |
| | AND THEIR USE FOR | | |
| | THE TREATMENT OF | | |
| | CANCER AND IMMUNE | | |
| | DISORDERS | | |
| | 2009 WO2009126934 | | |
| | RYAN, Maureen, SM . . . | | |
| | DETECTION AND | | |
| | TRATMENT OF | | |
| | PANCREATIC, OVARIAN | | |
| | AND OTHER CANCERS | | |
| | 2009 US20110300131 | | |
| | Ryan, Maureen; Sm . . . | | |
| | DETECTION AND | | |
| | TREATMENT OF | | |
| | PANCREATIC, OVARIAN | | |
| | AND OTHER CANCERS | | |
| | 2009 U.S. Pat. No. 9,120,854 Ryan, | | |
| | Maureen; Sm . . . | | |
| | Detection and | | |
| | treatment of | | |
| | pancreatic, ovarian and | | |
| | other cancers | | |
| | 2008 US20090074772 | | |
| | Law, Che-Leung; M . . . | | |
| | Anti-CD70 Antibody | | |
| | and Its Use for the | | |
| | Treatment of Cancer | | |
| | and Immune Disorders | | |
| | 2008 U.S. Pat. No. 9,051,372 Law, | | |
| | Che-Leung; M . . . Anti- | | |
| | CD70 antibody and its | | |
| | use for the treatment | | |
| | of cancer and immune | | |
| | disorders | | |
| | 2008 US20080138341 | | |
| | Law, Che-Leung; M . . . | | |
| | Anti-CD70 antibody | | |
| | and its use for the | | |
| | treatment of cancer | | |
| | and immune disorders | | |
| | 2008 US20080138343 | | |
| | Law, Che-Leung; M . . . | | |
| | ANTI-CD70 ANTIBODY | | |
| | AND ITS USE FOR THE | | |
| | TREATMENT OF | | |
| | CANCER AND IMMUNE | | |
| | DISORDERS | | |
| | 2008 U.S. Pat. No. 8,647,624 Law, | | |
| | Che-Leung; M . . . | | |
| | Treatment of immune | | |
| | disorders with anti- | | |
| | CD70 antibody | | |
| | 2007 U.S. Pat. No. 7,641,903 Law, | | |
| | Che-Leung; M . . . Anti- | | |
| | CD70 antibody and its | | |
| | use for the treatment | | |
| | and prevention of | | |
| | cancer and immune | | |
| | disorders | | |
| | 2007 US20080025989 | | |
| | Law, Che-Leung; M . . . | | |
| | ANTI-CD70 ANTIBODY- | | |
| | DRUG CONJUGATES | | |
| | AND THEIR USE FOR | | |
| | THE TREATMENT OF | | |
| | CANCER AND IMMUNE | | |
| | DISORDERS | | |
| | 2006 WO2006113909 | | |
| | MCDONAGH, Charlot . . . | | |
| | HUMANIZED ANTI- | | |
| | CD70 BINDING AGENTS | | |
| | AND USES THEREOF | | |
| | 2006 US20090148942 | | |
| | McDonagh, Charlot . . . | | |
| | HUMANIZED ANTI- | | |
| | CD70 BINDING AGENTS | | |
| | AND USES THEREOF | | |
| | 2006 U.S. Pat. No. 8,067,546 | | |
| | McDonagh, Charlot . . . | | |
| | Humanized anti-CD70 | | |
| | binding agents and | | |
| | uses thereof | | |
| | 2005 WO2006044643 | | |
| | LAW, Che-Leung; M . . . | | |
| | ANTI-CD70 ANTIBODY | | |
| | AND ITS USE FOR THE | | |
| | TREATMENT AND | | |
| | PREVENTION OF | | |
| | CANCER AND IMMUNE | | |
| | DISORDERS | | |
| | 2004 WO2004073656 | | |
| | LAW, Che-Leung; W . . . | | |
| | ANTI-CD70 ANTIBODY- | | |
| | DRUG CONJUGATES | | |
| | AND THEIR USE FOR | | |
| | THE TREATMENT OF | | |
| | CANCER AND IMMUNE | | |
| | DISORDERS | | |
| | 2004 U.S. Pat. No. 7,662,387 Law, | | |
| | Che-Leung; W . . . Anti- | | |
| | cd70 antibody-drug | | |
| | conjugates and their | | |
| | use for the treatment | | |
| | of cancer and immune | | |
| | disorders | | |
| Amgen patent anti- | 2020 US20210284748 | | Amgen |
| CD70/CD3 | Raum, Tobias; Blu . . . | | |
| | ANTIBODY | | |
| | CONSTRUCTS FOR | | |
| | CD70 AND CD3 | | |
| | 2016 WO2017021354 | | |
| | RAUM, Tobias, DEI . . . | | |
| | ANTIBODY | | |
| | CONSTRUCTS FOR | | |
| | CD70 AND CD3 | | |
| | 2016 US20170129961 | | |
| | Raum, Tobias; Blu . . . | | |
| | ANTIBODY | | |
| | CONSTRUCTS FOR | | |
| | CD70 AND CD3 | | |
| | 2016 U.S. Pat. No. 10,851,170 | | |
| | Raum, Tobias (Mun . . . | | |
| | Antibody constructs for | | |
| | CD70 and CD3 | | |
| Pfizer patent | 2022 US20230002498 | | Pfizer |
| anti-CD70/CD3 | Panowski, Siler; . . . | | |
| | ANTIBODIES SPECIFIC | | |
| | FOR CD70 AND THEIR | | |
| | USES | | |
| | 2019 US20190233529 | | |
| | Panowski, Siler; . . . | | |
| | ANTIBODIES SPECIFIC | | |
| | FOR CD70 AND THEIR | | |
| | USES | | |
| | 2019 WO2019152705 | | |
| | PANOWSKI, Siler, . . . | | |
| | ANTIBODIES SPECIFIC | | |
| | FOR CD70 AND THEIR | | |
| | USES | | |
| | 2019 U.S. Pat. No. 11,377,500 | | |
| | Panowski, Siler (. . . | | |
| | Antibodies specific for | | |
| | CD70 and their uses | | |

h. CD79b CAR

In some embodiments, the CAR is a CD79b CAR (“CD79b-CAR”), and in these embodiments, the polycistronic vector comprises an expression cassette that contains a nucleotide sequence encoding a CD79b CAR. CD79b is a pan B-cell linage marker and an important component of the B-cell receptor complex. CD79b is broadly expressed in normal B cells and B-cell malignancies and its expression is usually retained in CD19 negative tumors progressing after CD19-specific CAR T-cell therapy. In some embodiments, the CD79b CAR may comprise a signal peptide, an extracellular binding domain that specifically binds CD79b, a hinge domain, a transmembrane domain, an intracellular costimulatory domain, and/or an intracellular signaling domain in tandem.

In some embodiments, the signal peptide of the CD79b CAR comprises a CD8α signal peptide. In some embodiments, the CD8α signal peptide comprises or consists of an amino acid sequence set forth in SEQ ID NO:6 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:6. In some embodiments, the signal peptide comprises an IgK signal peptide. In some embodiments, the IgK signal peptide comprises or consists of an amino acid sequence set forth in SEQ ID NO:7 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:7. In some embodiments, the signal peptide comprises a GMCSFR-α or CSF2RA signal peptide. In some embodiments, the GMCSFR-α or CSF2RA signal peptide comprises or consists of an amino acid sequence set forth in SEQ ID NO:8 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:8.

In some embodiments, the extracellular binding domain of the CD79b CAR is specific to CD79b, for example, human CD79b. The extracellular binding domain of the GPRC5D CAR can be codon-optimized for expression in a host cell or to have variant sequences to increase functions of the extracellular binding domain. In some embodiments, the extracellular binding domain comprises an immunogenically active portion of an immunoglobulin molecule, for example, an scFv.

In some embodiments, the extracellular binding domain of the CD79b CAR is derived from an antibody specific to CD79b, including, for example, any of the antibodies or CARs disclosed in Table 26, the references cited in which are incorporated by reference in their entireties herein. In any of these embodiments, the extracellular binding domain of the CD79b CAR can comprise or consist of the V_{H}, the V_{L}, and/or one or more CDRs of any of the antibodies as described herein, including in Table 26.

In some embodiments, the extracellular binding domain of the CD79b CAR comprises an scFv derived from the any of the antibodies or CARs disclosed in Table 26, optionally comprising the heavy chain variable region (V_{H}) and the light chain variable region (V_{L}) of one of the antibodies or CARs, connected by a linker. In some embodiments, the linker is a 3×G₄S linker. In other embodiments, the Whitlow linker may be used instead. In some embodiments, the CD79b-specific scFv comprises or consists of the scFv of an antibody or CAR disclosed in Table 26, or an amino acid sequence that is at least 80% identical (e.g., at least 80%, at least 85%, at least 90%, at least 95%, at least 96%, at least 97%, at least 98%, at least 99%, or 100% identical) to the amino acid sequence of the scFv of an antibody or CAR disclosed in Table 26. In some embodiments, the CD79b-specific scFv may comprise one or more CDRs having amino acid sequences of the CDRs of an antibody or CAR disclosed in Table 26. In some embodiments, the CD79b-specific scFv may comprise a heavy chain with one or more CDRs having amino acid sequences of the CDRs of an antibody or CAR disclosed in Table 26. In some embodiments, the CD79b-specific scFv may comprise a light chain with one or more CDRs having amino acid sequences of the CDRs of an antibody or CAR disclosed in Table 26. In any of these embodiments, the CD79b-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the CD79b CAR comprises or consists of the one or more CDRs as described herein.

TABLE 26
Exemplary CD79b antigen binding domains

| Name | Antigen | Company | Reference |
|---|---|---|---|
| | CD79b | Astellas | US20170226207 |
| CNTY-102 | CD19, CD79b | Century | |
| | CD79b | Autolus | WO2019220110 |
| iladatuzumab | CD79b | Roche | US20140356352 |
| vedotin | | | |
| | CD79b | Janssen Biotech | US20210145878 |
| | CD79b | NewBio Thera | US20210388082 |
| polatuzumab | CD79b | Genentech, Roche, Seattle | US20160159906 |
| vedotin-piiq | | Genetics | |
| | CD79b | Tuojie Biotech | WO2020156439 |
| | CD79b | U.Texas | US20210317209 |
| | CD79b | UCB | US20180201678 |
| | CD79b, CD19 | Mass. General Hosp. | WO2020124021 |
| MGD010 | CD79b, Fc-gamma-R2B | MacroGenics, Provention | US20190322741 |
| | (CD32b) | Bio, Takeda | |
| | CD79b, CD79A | Nepenthe | U.S. Pat No. 11,078,276 |
| | CD79b, CD22 | UCB | WO2016009030 |
| | CD79b, CD45 | UCB | WO2016009029 |

In some embodiments, the hinge domain of the CD79b CAR comprises a CD8α hinge domain, for example, a human CD8α hinge domain. In some embodiments, the CD8α hinge domain comprises or consists of an amino acid sequence set forth in SEQ ID NO:9 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:9. In some embodiments, the hinge domain comprises a CD28 hinge domain, for example, a human CD28 hinge domain. In some embodiments, the CD28 hinge domain comprises or consists of an amino acid sequence set forth in SEQ ID NO:10 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:10. In some embodiments, the hinge domain comprises an IgG4 hinge domain, for example, a human IgG4 hinge domain. In some embodiments, the IgG4 hinge domain comprises or consists of an amino acid sequence set forth in SEQ ID NO:11 or SEQ ID NO:12, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:11 or SEQ ID NO:12. In some embodiments, the hinge domain comprises a IgG4 hinge-Ch2-Ch3 domain, for example, a human IgG4 hinge-Ch2-Ch3 domain. In some embodiments, the IgG4 hinge-Ch2-Ch3 domain comprises or consists of an amino acid sequence set forth in SEQ ID NO:13 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:13.

In some embodiments, the transmembrane domain of the CD79b CAR comprises a CD8α transmembrane domain, for example, a human CD8α transmembrane domain. In some embodiments, the CD8α transmembrane domain comprises or consists of an amino acid sequence set forth in SEQ ID NO:14 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:14. In some embodiments, the transmembrane domain comprises a CD28 transmembrane domain, for example, a human CD28 transmembrane domain. In some embodiments, the CD28 transmembrane domain comprises or consists of an amino acid sequence set forth in SEQ ID NO:15 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:15.

In some embodiments, the intracellular costimulatory domain of the CD79b CAR comprises a 4-1BB costimulatory domain, for example, a human 4-1BB costimulatory domain. In some embodiments, the 4-1BB costimulatory domain comprises or consists of an amino acid sequence set forth in SEQ ID NO:16 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:16. In some embodiments, the intracellular costimulatory domain comprises a CD28 costimulatory domain, for example, a human CD28 costimulatory domain. In some embodiments, the CD28 costimulatory domain comprises or consists of an amino acid sequence set forth in SEQ ID NO:17 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:17.

In some embodiments, the intracellular signaling domain of the CD79b CAR comprises a CD3 zeta (ζ) signaling domain, for example, a human CD3ζ signaling domain. In some embodiments, the CD3ζ signaling domain comprises or consists of an amino acid sequence set forth in SEQ ID NO:18 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:18.

In some embodiments, the polycistronic vector comprises an expression cassette that contains a nucleotide sequence encoding a CD79b CAR, including, for example, a CD79b CAR comprising the CD79b-specific scFv having sequences of an antibody or CAR disclosed in Table 26, the CD8α hinge domain of SEQ ID NO:9, the CD8α transmembrane domain of SEQ ID NO:14, the 4-1BB costimulatory domain of SEQ ID NO:16, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD79b CAR, including, for example, a CD79b CAR comprising the CD79b-specific scFv having sequences of an antibody or CAR disclosed in Table 26, the CD28 hinge domain of SEQ ID NO:10, the CD8α transmembrane domain of SEQ ID NO:14, the 4-1BB costimulatory domain of SEQ ID NO:16, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD79b CAR, including, for example, a CD79b CAR comprising the CD79b-specific scFv having sequences of an antibody or CAR disclosed in Table 26, the IgG4 hinge domain of SEQ ID NO:11 134 or SEQ ID NO:12, the CD8α transmembrane domain of SEQ ID NO:14, the 4-1BB costimulatory domain of SEQ ID NO:16, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD79b CAR, including, for example, a CD79b CAR comprising the CD79b-specific scFv having sequences of an antibody or CAR disclosed in Table 26, the CD8α hinge domain of SEQ ID NO:9, the CD28 transmembrane domain of SEQ ID NO:15, the 4-1BB costimulatory domain of SEQ ID NO:16, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD79b CAR, including, for example, a CD79b CAR comprising the CD79b-specific scFv having sequences of an antibody or CAR disclosed in Table 26, the CD28 hinge domain of SEQ ID NO:10, the CD28 transmembrane domain of SEQ ID NO:15, the 4-1BB costimulatory domain of SEQ ID NO:16, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD79b CAR, including, for example, a CD79b CAR comprising the CD79b-specific scFv having sequences of an antibody or CAR disclosed in Table 26, the IgG4 hinge domain of SEQ ID NO:11 or SEQ ID NO:12, the CD28 transmembrane domain of SEQ ID NO:15, the 4-1BB costimulatory domain of SEQ ID NO:16, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, a polynucleotide provided herein comprises a nucleotide sequence encoding a CD79B CAR, a variable domain of a CD79B CAR, or a set of CDRs (HCDR 1, 2, and 3 and LCDR 1, 2, and 3) that encodes a CD79B CAR as set forth in TABLE 27 below or a variable domain of a CD79B CAR or a set of CDRs (HCDR 1, 2, and 3 and LCDR 1, 2, and 3) therefrom. In some embodiments, a polynucleotide provided herein comprises a nucleotide sequence encoding a CD79B CAR, a variable domain of a CD79B CAR, or a set of CDRs (HCDR 1, 2, and 3 and LCDR 1, 2, and 3) that having at least 80% (e.g., at least 80%, at least 85%, at least 90%, at least 95%, at least 96%, at least 97%, at least 98%, at least 99%, or 100% identical) to a CD79B CAR as set forth in TABLE 27 below or a variable domain of a CD79B CAR or a set of CDRs (HCDR 1, 2, and 3 and LCDR 1, 2, and 3) therefrom, respectively.

TABLE 27
Exemplary CD79b antigen binding domains

| Antibody Name | Patents | Publications | Company |
|---|---|---|---|
| ADC Therapeutics | 2022 WO2023274974 | | ADC Therapeutics |
| patent anti-CD79b | VAN BERKEL, Patri . . . | | |
| | COMBINATION | | |
| | THERAPY USING | | |
| | ANTIBODY-DRUG | | |
| | CONJUGATES | | |
| Astellas patent anti- | 2015 WO2016021621 | | Astellas |
| CD79b | YAMAJUKU, Daisuke . . . | | |
| | NEW ANTI-HUMAN Igβ | | |
| | ANTIBODY | | |
| | 2015 US20170226207 | | |
| | YAMAJUKU, Daisuke . . . | | |
| | NOVEL ANTI-HUMAN | | |
| | IgB ANTIBODY | | |
| | 2015 U.S. Pat. No. 10,316,086 | | |
| | Yamajuku, Daisuke . . . | | |
| | Anti-human Igβ | | |
| | antibody | | |
| Autolus patent anti- | 2019 WO2019220110 | | Autolus |
| CD79 CAR | PULÉ, Martin, COR . . . A | | |
| | CD79-SPECIFIC | | |
| | CHIMERIC ANTIGEN | | |
| | RECEPTOR | | |
| iladatuzumab vedotin | 2021 WO2022228705 | 2015 NCT02453087 | Roche |
| | DIMIER, Natalie | Phase 1 A Study of | |
| | DOSING FOR | Escalating Doses of | |
| | COMBINATION | DCDS0780A in Patients | |
| | TREATMENT WITH | With Relapsed or | |
| | ANTI-CD20/ANTI-CD3 | Refractory B-cell Non- | |
| | BISPECIFIC ANTIBODY | Hodgkin's Lymphoma | |
| | AND ANTI-CD79B | 2022 Herrera A F, | |
| | ANTIBODY DRUG | Patel . . . Anti-CD79B | |
| | CONJUGATE | Antibody-Drug | |
| | | Conjugate DCDS0780A | |
| | | in Patients with B-Cell | |
| | | Non-Hodgkin | |
| | | Lymphoma: Phase 1 | |
| | | Dose-Escalation Study. | |
| | | ASH 2017 A Phase I | |
| | | Study of the Anti- | |
| | | CD79b THIOMABTM- | |
| | | Drug Conjugate | |
| | | DCDS0780A in Patients | |
| | | (pts) with Relapsed or | |
| | | Refractory B-Cell Non- | |
| | | Hodgkin's Lymphoma | |
| | | (B-NHL) Alex F. | |
| | | Herrera, . . . | |
| Janssen patent anti- | 2022 WO2022254292 | | Janssen Biotech |
| CD79b CAR | GRUGAN, Katharine D. | | |
| | ANTI-IDIOTYPIC | | |
| | ANTIBODIES AGAINST | | |
| | ANTI-CD79B | | |
| | ANTIBODIES | | |
| | 2022 WO2022192649 | | |
| | ZHENG, Songmao, E . . . | | |
| | USES OF CD79B | | |
| | ANTIBODIES FOR | | |
| | AUTOIMMUNE | | |
| | THERAPEUTIC | | |
| | APPLICATIONS | | |
| NewBio Thera patent | 2019 WO2020088587 | | NewBio Thera |
| anti-CD79b | HAN, Nianhe, SONG . . . | | |
| | Anti-CD79b Antibodies, | | |
| | Drug Conjugates, and | | |
| | Applications thereof | | |
| | 2019 US20210388082 | | |
| | HAN, Nianhe; SONG . . . | | |
| | Anti-CD79b Antibodies, | | |
| | Drug Conjugates, and | | |
| | Applications Thereof | | |
| polatuzumab vedotin- | 2022 WO2022241235 | 2022 NCT05633615 | Genentech Roche |
| piiq | O'HEAR, Carol, El . . . | Phase 2 Testing Drug | Seattle Genetics |
| | METHODS FOR | Treatments After CAR | |
| | TREATMENT OF CD20- | T-cell Therapy in | |
| | POSITIVE | Patients With Diffuse | |
| | PROLIFERATIVE | Large B-cell Lymphoma | |
| | DISORDER WITH | That Has Come Back or | |
| | MOSUNETUZUMAB | Not Responded to | |
| | AND POLATUZUMAB | Treatment | |
| | VEDOTIN | 2022 NCT05410418 | |
| | 2022 WO2022241446 | Phase 2 | |
| | HIRATA, Jamie Harue | Mosunetuzumab and | |
| | METHODS OF USING | Polatuzumab Vedotin | |
| | ANTI-CD79B | for Untreated Follicular | |
| | IMMUNOCONJUGATES | Lymphoma | |
| | TO TREAT DIFFUSE | 2022 NCT05260957 | |
| | LARGE B-CELL | Phase 2 CAR-T Cell | |
| | LYMPHOMA | Therapy, | |
| | 2022 US20220380480 | Mosunetuzumab and | |
| | LECHNER, Katharin . . . | Polatuzumab for | |
| | DOSING FOR | Treatment of | |
| | COMBINATION | Refractory/Relapsed | |
| | TREATMENT WITH | Aggressive Non- | |
| | ANTI-CD20/ANTI-CD3 | Hodgkin's Lymphoma | |
| | BISPECIFIC ANTIBODY | (NHL). | |
| | AND ANTI-CD79B | 2022 NCT05169658 | |
| | ANTIBODY DRUG | Phase 2 | |
| | CONJUGATE | Mosunetuzumab With | |
| | 2022 US20220251197 | or Without | |
| | CHEN, Yvonne; DEN . . . | Polatuzumab Vedotin | |
| | ANTI-CD79B | and Obinutuzumab for | |
| | ANTIBODIES AND | the Treatment of | |
| | IMMUNOCONJUGATES | Untreated Indolent B- | |
| | AND METHODS OF USE | Cell Non-Hodgkin | |
| | 2021 US20220125942 | Lymphoma | |
| | MUSICK, Lisa; HIR . . . | 2022 NCT05171647 | |
| | METHODS OF USING | Phase 3 A Study | |
| | ANTI-CD79b | Evaluating Efficacy and | |
| | IMMUNOCONJUGATES | Safety of | |
| | TO TREAT FOLLICULAR | Mosunetuzumab in | |
| | LYMPHOMA | Combination With | |
| | 2021 US20220153842 | Polatuzumab Vedotin | |
| | LI, Chi-Chung; O' . . . | Compared to | |
| | DOSING FOR | Rituximab in | |
| | TREATMENT WITH | Combination With | |
| | ANTI-CD20/ANTI-CD3 | Gemcitabine Plus | |
| | BISPECIFIC ANTIBODIES | Oxaliplatin in | |
| | AND ANTI-CD79B | Participants With | |
| | ANTIBODY DRUG | Relapsed or Refractory | |
| | CONJUGATES | Aggressive B-Cell Non- | |
| | 2021 US20220031861 | Hodgkin's Lymphoma | |
| | HIRATA, Jamie Har . . . | 2021 NCT04844866 | |
| | COMBINATION | Phase 2 Efficacy and | |
| | THERAPY OF DIFFUSE | Safety of MB- | |
| | LARGE B-CELL | CART2019.1 vs. SoC in | |
| | LYMPHOMA | Lymphoma Patients | |
| | COMPRISING AN ANTI- | 2021 NCT04739813 | |
| | CD79B | Phase 1 Phase 1 Study | |
| | IMMUNOCONJUGATES, | of Venetoclax, | |
| | AN ALKYLATING AGENT | Ibrutinib, Prednisone, | |
| | AND AN ANTI-CD20 | Obinutuzumab, and | |
| | ANTIBODY | Revlimid in | |
| | 2021 US20210346352 | Combination With | |
| | POLSON, Andrew; Y . . . | Polatuzumab (ViPOR-P) | |
| | METHODS OF USING | in Relapsed/Refractory | |
| | ANTI-CD79b | B-cell Lymphoma | |
| | IMMUNOCONJUGATES | 2021 NCT04679012 | |
| | 2021 WO2021217051 | Phase 2 Polatuzumab | |
| | HIRATA, Jamie Har . . . | Vedotin in | |
| | METHODS OF USING | Combination With | |
| | ANTI-CD79B | Chemotherapy in | |
| | IMMUNOCONJUGATES | Subjects With Richter's | |
| | 2021 US20220023437 | Transformation | |
| | CHEN, Yvonne; DEN . . . | 2020 NCT04659044 | |
| | HUMANIZED ANTI- | Phase 2 Polatuzumab | |
| | CD79B ANTIBODIES | Vedotin, Venetoclax, | |
| | AND | and Rituximab and | |
| | IMMUNOCONJUGATES | Hyaluronidase Human | |
| | AND METHODS OF USE | for the Treatment of | |
| | 2021 US20220040153 | Relapsed or Refractory | |
| | POLSON, Andrew; Y . . . | Mantle Cell Lymphoma | |
| | METHODS OF USING | 2020 NCT04665765 | |
| | ANTI-CD79b | Phase 2 Polatuzumab | |
| | IMMUNOCONJUGATES | Vedotin, Rituximab, | |
| | 2020 US20210138065 | Ifosfamide, | |
| | KLEIN, Christian; . . . | Carboplatin, and | |
| | COMBINATION | Etoposide (PolaR-ICE) | |
| | THERAPY OF AN | as Initial Salvage | |
| | AFUCOSYLATED CD20 | Therapy for the | |
| | ANTIBODY WITH A | Treatment of | |
| | CD79b ANTIBODY- | Relapsed/Refractory | |
| | DRUG CONJUGATE | Diffuse Large B-Cell | |
| | 2020 US20210115141 | Lymphoma | |
| | HERNANDEZ | 2020 NCT04479267 | |
| | MONTALV . . . METHODS | Phase 2 Polatuzumab | |
| | OF USING ANTI-CD79B | Vedotin and | |
| | IMMUNOCONJUGATES | Combination | |
| | TO TREAT DIFFUSE | Chemotherapy for the | |
| | LARGE B-CELL | Treatment of | |
| | LYMPHOMA | Previously Untreated | |
| | 2020 WO2021076196 | Double or Triple Hit | |
| | HERNANDEZ | Lymphoma | |
| | MONTALV . . . METHODS | 2020 NCT04332822 | |
| | OF USING ANTI-CD79B | Phase 3 A Randomized, | |
| | IMMUNOCONJUGATES | Multicenter, Phase III | |
| | TO TREAT DIFFUSE | Trial Comparing | |
| | LARGE B-CELL | Treatment With R- | |
| | LYMPHOMA | mini-CHOP With R- | |
| | 2020 WO2020232169 | mini-CHP + | |
| | MUSICK, Lisa, HIR . . . | Polatuzumab Vedotin | |
| | METHODS OF USING | in Patients With Diffuse | |
| | ANTI-CD79B | Large Cell B Cell | |
| | IMMUNOCONJUGATES | Lymphoma | |
| | TO TREAT FOLLICULAR | 2020 NCT04231877 | |
| | LYMPHOMA | Phase 1 Polatuzumab | |
| | 2019 US20200246438 | Vedotin and | |
| | Ceol, Craig Josep . . . | Combination | |
| | TARGETING GDF6 AND | Chemotherapy for the | |
| | BMP SIGNALING FOR | Treatment of | |
| | ANTI-MELANOMA | Untreated Aggressive | |
| | THERAPY | Large B-cell Lymphoma | |
| | 2019 US20200079852 | 2020 NCT04236141 | |
| | CHEN, Yvonne; DEN . . . | Phase 3 A Study to | |
| | ANTI-CD79B | Evaluate the Efficacy | |
| | ANTIBODIES AND | and Safety of | |
| | IMMUNOCONJUGATES | Polatuzumab Vedotin | |
| | AND METHODS OF USE | in Combination With | |
| | 2019 WO2019200322 | Bendamustine and | |
| | PATEL, Ankit R., . . . | Rituximab Compared | |
| | STABLE ANTI-CD79B | With Bendamustine | |
| | IMMUNOCONJUGATE | and Rituximab Alone in | |
| | FORMULATIONS | Chinese Patients With | |
| | 2019 US20190201382 | Relapsed or Refractory | |
| | POLSON, Andrew; Y . . . | Diffuse Large B-cell | |
| | METHODS OF USING | Lymphoma (R/R | |
| | ANTI-CD79b | DLBCL). | |
| | IMMUNOCONJUGATES | 2019 NCT04624893 A | |
| | 2019 U.S. Pat. No. 11,000,510 | Multicenter, | |
| | Polson, Andrew (S . . . | Retrospective | |
| | Methods of using anti- | Observational Study to | |
| | CD79b | Evaluate the | |
| | immunoconjugates | Effectiveness and | |
| | 2018 WO2020117257 | Safety of Polatuzumab | |
| | POLSON, Andrew, Y . . . | Vedotin | |
| | COMBINATION | 2019 NCT04182204 | |
| | THERAPY OF DIFFUSE | Phase 3 A Study to | |
| | LARGE B-CELL | Evaluate the Safety and | |
| | LYMPHOMA | Efficacy of | |
| | COMPRISING AN ANTI- | Polatuzumab Vedotin | |
| | CD79B | in Combination With | |
| | IMMUNOCONJUGATES, | Rituximab, | |
| | AN ALKYLATING AGENT | Gemcitabine and | |
| | AND AN ANTI-CD20 | Oxaliplatin Compared | |
| | ANTIBODY | to Rituximab, | |
| | 2018 US20180344848 | Gemcitabine and | |
| | Klein, Christian; . . . | Oxaliplatin Alone in | |
| | COMBINATION | Participants With | |
| | THERAPY OF AN | Relapsed or Refractory | |
| | AFUCOSYLATED CD20 | Diffuse Large B-Cell | |
| | ANTIBODY WITH A | Lymp . . . | |
| | CD79b ANTIBODY- | 2018 NCT03671018 | |
| | DRUG CONJUGATE | Phase 1/Phase 2 A | |
| | 2018 US20180327492 | Study to Evaluate the | |
| | Sun, Liping L.; C . . . ANTI- | Safety and Efficacy of | |
| | CD79b ANTIBODIES | Mosunetuzumab | |
| | AND METHODS OF USE | (BTCT4465A) in | |
| | 2018 U.S. Pat. No. 10,941,199 Sun, | Combination With | |
| | Liping L. (S . . . Anti- | Polatuzumab Vedotin | |
| | CD79b antibodies and | in B-Cell Non-Hodgkin | |
| | methods of use | Lymphoma | |
| | 2018 USRE048558 Anti- | 2018 NCT03677141 | |
| | CD79B antibo . . . Anti- | Phase 1/Phase 2 A | |
| | CD79B antibodies and | Phase Ib/II Study | |
| | immunoconjugates and | Investigating the | |
| | methods of use | Safety, Tolerability, | |
| | 2017 US20180133315 | Pharmacokinetics, and | |
| | Klein, Christian; . . . | Efficacy of | |
| | COMBINATION | Mosunetuzumab | |
| | THERAPY OF AN | (BTCT4465A) in | |
| | AFUCOSYLATED CD20 | Combination With | |
| | ANTIBODY WITH A | CHOP or CHP- | |
| | CD79b ANTIBODY- | Polatuzumab Vedotin | |
| | DRUG CONJUGATE | in Participants With B- | |
| | 2017 US20180201679 | Cell Non-Hodgkin | |
| | Chen, Yvonne; Den . . . | Lymphoma | |
| | HUMANIZED ANTI- | 2017 NCT03274492 | |
| | CD79B ANTIBODIES | Phase 3 A Study | |
| | AND | Comparing the Efficacy | |
| | IMMUNOCONJUGATES | and Safety of | |
| | AND METHODS OF USE | Polatuzumab Vedotin | |
| | 2017 U.S. Pat. No. 10,981,987 | With Rituximab- | |
| | Chen, Yvonne (San . . . | Cyclophosphamide, | |
| | Humanized anti-CD79b | Doxorubicin, and | |
| | antibodies and | Prednisone (R-CHP) | |
| | immunoconjugates and | Versus Rituximab- | |
| | methods of use | Cyclophosphamide, | |
| | 2017 US20180015179 | Doxorubicin, | |
| | Polakis, Paul; Po . . . | Vincristine, and | |
| | ANTI-CD79B | Prednisone (R-CHOP) in | |
| | ANTIBODIES AND | Participants With | |
| | IMMUNOCONJUGATES | Diffuse Larg . . . | |
| | 2017 US20180169259 | 2016 NCT02729896 | |
| | Polakis, Paul; Po . . . | Phase 1/Phase 2 A | |
| | ANTI-CD79B | Study of | |
| | ANTIBODIES AND | Obinutuzumab, | |
| | IMMUNOCONJUGATES | Polatuzumab Vedotin, | |
| | 2017 US20170304438 | and Atezolizumab in | |
| | Polson, Andrew; Y . . . | Relapsed or Refractory | |
| | METHODS OF USING | Follicular Lymphoma | |
| | ANTI-CD79b | (FL) or Diffuse Large B- | |
| | IMMUNOCONJUGATES | Cell Lymphoma (DLBCL) | |
| | 2016 US20170058032 | 2015 NCT02600897 | |
| | CHEN, Yvonne; DEN . . . | Phase 1 A Study of | |
| | ANTI-CD79B | Obinutuzumab, | |
| | ANTIBODIES AND | Polatuzumab Vedotin, | |
| | IMMUNOCONJUGATES | and Lenalidomide in | |
| | AND METHODS OF USE | Relapsed or Refractory | |
| | 2016 U.S. Pat. No. 10,544,218 | Follicular Lymphoma | |
| | Chen, Yvonne (San . . . | (FL) or Diffuse Large B- | |
| | Anti-CD79B antibodies | Cell Lymphoma (DLBCL) | |
| | and immunoconjugates | 2014 NCT02257567 | |
| | and methods of use | Phase 2 A Study of | |
| | 2016 WO2016205176 | Polatuzumab Vedotin | |
| | POLAKIS, Paul, DR . . . | (DCDS4501A) in | |
| | ANTIBODIES AND | Combination With | |
| | IMMUNOCONJUGATES | Rituximab or | |
| | 2015 US20160159906 | Obinutuzumab Plus | |
| | Sun, Liping L.; C . . . ANTI- | Bendamustine in | |
| | CD79b ANTIBODIES | Patients With Relapsed | |
| | AND METHODS OF USE | or Refractory Follicular | |
| | 2015 WO2016090210 | or Diffuse Large B-Cell | |
| | SUN, Liping L., . . . ANTI- | Lymphoma | |
| | CD79b ANTIBODIES | 2013 NCT01992653 | |
| | AND METHODS OF USE | Phase 1 A Study of | |
| | 2015 U.S. Pat. No. 9,975,949 Sun, | DCDS4501A in | |
| | Liping L.; C . . . Anti- | Combination With | |
| | CD79b antibodies and | Rituximab, | |
| | methods of use | Cyclophosphamide, | |
| | 2015 US20160082120 | Doxorubicin and | |
| | Polson, Andrew; Y . . . | Prednisone in Patients | |
| | METHODS OF USING | With B-Cell Non- | |
| | ANTI-CD79b | Hodgkin's Lymphoma | |
| | IMMUNOCONJUGATES | 2012 NCT01691898 | |
| | 2015 WO2016049214 | Phase 2 A Study of | |
| | POLSON, Andrew, . . . | DCDT2980S in | |
| | METHOD OF USING | Combination With | |
| | ANTI-CD79b | MabThera/Rituxan or | |
| | IMMUNOCONJUGATES | DCDS4501A in | |
| | 2015 US20150314016 | Combination With | |
| | CHEN, Yvonne; DEN . . . | MabThera/Rituxan in | |
| | ANTI-CD79B | Patients With Non- | |
| | ANTIBODIES AND | Hodgkin's Lymphoma | |
| | IMMUNOCONJUGATES | 2011 NCT01290549 | |
| | AND METHODS OF USE | Phase 1 A Study of | |
| | 2015 U.S. Pat. No. 9,896,506 Chen, | Escalating Doses of | |
| | Yvonne; Den . . . Anti- | DCDS4501A in Patients | |
| | CD79B antibodies and | With Relapsed or | |
| | immunoconjugates and | Refractory B-Cell Non | |
| | methods of use | Hodgkin's Lymphoma | |
| | 2014 WO2014177615 | and Chronic | |
| | KLEIN, Christian, . . . | Lymphocytic Leukemia | |
| | COMBINATION | 1999 NCT03199066 | |
| | THERAPY OF AN | Non-Hodgkin | |
| | AFUCOSYLATED CD20 | Lymphoma - | |
| | ANTIBODY WITH A | Observational | |
| | CD79b ANTIBODY- | Epidemiological and | |
| | DRUG CONJUGATE | Clinical Study (NiHiL) | |
| | 2014 US20140356352 | 2022 Flowers C, Tilly . . . | |
| | Klein, Christian; . . . | ABCL-073 Polatuzumab | |
| | COMBINATION | Vedotin Plus | |
| | THERAPY OF AN | Rituximab, | |
| | AFUCOSYLATED CD20 | Cyclophosphamide, | |
| | ANTIBODY WITH A | Doxorubicin, and | |
| | CD79b ANTIBODY- | Prednisone (Pola-R- | |
| | DRUG CONJUGATE | CHP) Versus Rituximab, | |
| | 2014 US20140335107 | Cyclophosphamide, | |
| | Chen, Yvonne; Den . . . | Doxorubicin, | |
| | ANTI-CD79B | Vincristine and | |
| | ANTIBODIES AND | Prednisone (R-CHOP) | |
| | IMMUNOCONJUGATES | Therapy in Patients | |
| | AND METHODS OF USE | With Previously | |
| | 2013 US20140099260 | Untreated Diffuse | |
| | Chen, Yvonne; Den . . . | Large B-Cell Lymphoma | |
| | Anti-CD79B Antibodies | (DLBCL): Results From | |
| | and Immunoconjugates | the Phase III POLARIX | |
| | and Methods of Use | Study. | |
| | 2013 US20190276550 | 2022 Matasar M, | |
| | Chen, Yvonne; Den . . . | Masaqu . . . ABCL-101 | |
| | Anti-CD79B Antibodies | Cost-Effectiveness of | |
| | and Immunoconjugates | Polatuzumab Vedotin | |
| | and Methods of Use | in Previously Untreated | |
| | 2013 U.S. 10,494,432 | Diffuse Large B-Cell | |
| | Chen, Yvonne (San . . . | Lymphoma. | |
| | Anti-CD79B antibodies | 2022 Oka S, Ono K, | |
| | and immunoconjugates | Noh . . . Effective | |
| | and methods of use | treatment with | |
| | 2013 WO2014011521 | polatuzumab vedotin | |
| | POLAKIS, Paul, PO . . . | of relapsed and | |
| | IMMUNOCONJUGATES | refractory primary | |
| | COMPRISING ANTI - | cutaneous diffuse large | |
| | CD79B ANTIBODIES | B-cell lymphoma leg | |
| | 2013 WO2014011519 | type. | |
| | POLAKIS, Paul, PO . . . | 2022 Tarantelli C, Ber . . . | |
| | IMMUNOCONJUGATES | United we stand: | |
| | COMPRISING ANTI- | Double targeting of | |
| | CD79B ANTIBODIES | CD79B and CD20 in | |
| | 2013 US20140030280 | diffuse large B-cell | |
| | Polakis, Paul; Po . . . | lymphoma. | |
| | ANTI-CD79B | 2022 Kawasaki N, | |
| | ANTIBODIES AND | Nishi . . . The molecular | |
| | IMMUNOCONJUGATES | rationale for the | |
| | 2013 US20140030282 | combination of | |
| | Polakis, Paul; Po . . . | polatuzumab vedotin | |
| | ANTI-CD79B | plus rituximab in | |
| | ANTIBODIES AND | diffuse large B-cell | |
| | IMMUNOCONJUGATES | lymphoma. | |
| | 2011 US20120148600 | 2022 Varma G, Wang | |
| | Chen, Yvonne; Den . . . | J, . . . Polatuzumab vedotin | |
| | Anti-CD79B Antibodies | in relapsed/refractory | |
| | and Immunoconjugates | aggressive B-cell | |
| | and Methods of Use | lymphoma. | |
| | 2011 U.S. Pat. No. 8,691,531 Chen, | 2022 Zengarini C, | |
| | Yvonne; Den . . . Anti- | Misc . . . Who is the | |
| | CD79B antibodies and | culprit? A toxic | |
| | immunoconjugates and | epidermal necrolysis | |
| | methods of use | case in a patient | |
| | 2010 US20110135667 | treated with rituximab | |
| | CHEN, YVONNE; DEN . . . | plus polatuzumab. | |
| | ANTI-CD79B | 2022 Vodicka P, | |
| | ANTIBODIES AND | Beneso . . . Polatuzumab | |
| | IMMUNOCONJUGATES | vedotin plus | |
| | AND METHODS OF USE | bendamustine and | |
| | 2010 U.S. Pat. No. 8,545,850 Chen, | rituximab in patients | |
| | Yvonne; Den . . . Anti- | with | |
| | CD79B antibodies and | relapsed/refractory | |
| | immunoconjugates and | diffuse large B-cell | |
| | methods of use | lymphoma in the real | |
| | 2009 WO2009099728 | world. | |
| | CHEN, Yvonne; DEN . . . | 2022 Northend M, | |
| | ANTI-CD79B | Wilso . . . Results of a | |
| | ANTIBODIES AND | United Kingdom real- | |
| | IMMUNOCONJUGATES | world study of | |
| | AND METHODS OF USE | polatuzumab vedotin, | |
| | 2009 US20100215669 | bendamustine, and | |
| | CHEN, Yvonne; Den . . . | rituximab for | |
| | ANTI-CD79B | relapsed/refractory | |
| | ANTIBODIES AND | DLBCL. | |
| | IMMUNOCONJUGATES | 2022 Gouni S, | |
| | AND METHODS OF USE | Rosentha . . . A | |
| | 2009 U.S. Pat. No. 8,722,857 Chen, | Multicenter | |
| | Yvonne; Den . . . Anti- | Retrospective Study of | |
| | CD79B antibodies and | Polatuzumab Vedotin | |
| | immunoconjugates and | in Patients with Large | |
| | methods of use | B-cell Lymphoma After | |
| | 2008 WO2009012256 | CAR T-cell Therapy. | |
| | CHEN, Yvonne; DEN . . . | 2022 Takakuwa T, | |
| | HUMANIZED ANTI- | Okaya . . . Polatuzumab | |
| | CD79B ANTIBODIES | vedotin combined with | |
| | AND | rituximab- | |
| | IMMUNOCONJUGATES | bendamustine | |
| | AND METHODS OF USE | immediately before | |
| | 2008 WO2009012268 | stem cell mobilization | |
| | CHEN, Yvonne; DEN . . . | in relapsed diffuse | |
| | ANTI-CD79B | large B-cell lymphoma. | |
| | ANTIBODIES AND | 2022 Avivi I, Perry C, . . . | |
| | IMMUNOCONJUGATES | Polatuzumab-based | |
| | AND METHODS OF USE | regimen or CAR T cell | |
| | 2008 U.S. Pat. No. 8,088,378 Chen, | for patients with | |
| | Yvonne; Den . . . Anti- | refractory/relapsed | |
| | CD79B antibodies and | DLBCL-a matched | |
| | immunoconjugates and | cohort analysis. | |
| | methods of use | 2021 Tilly H, | |
| | 2008 US20090068202 | Morschha . . . | |
| | Chen, Yvonne; Den . . . | Polatuzumab Vedotin | |
| | Humanized Anti-CD79B | in Previously Untreated | |
| | Antibodies and | Diffuse Large B-Cell | |
| | Immunoconjugates and | Lymphoma. | |
| | Methods of Use | 2021 Wang Y W, Tsai | |
| | 2008 U.S. Pat. No. 9,845,355 Chen, | X C, . . . Polatuzumab | |
| | Yvonne; Den . . . | vedotin-based salvage | |
| | Humanized anti-CD79b | immunochemotherapy | |
| | antibodies and | as third-line or beyond | |
| | immunoconjugates and | treatment for patients | |
| | methods of use | with diffuse large B-cell | |
| | | lymphoma: a real- | |
| | | world experience. | |
| | | 2021 Wu S Y, Fang P, | |
| | | Hu . . . Concurrent | |
| | | Radiation Therapy With | |
| | | the Antibody-Drug | |
| | | Conjugates | |
| | | Brentuximab Vedotin | |
| | | and Polatuzumab | |
| | | Vedotin. | |
| | | 2021 Liebers N, Duell . . . | |
| | | Polatuzumab vedotin | |
| | | as a salvage and | |
| | | bridging treatment in | |
| | | relapsed or refractory | |
| | | large B-cell | |
| | | lymphomas. | |
| | | 2021 Wu J Q, Liu Y Y, Li . . . | |
| | | [Cohort study of | |
| | | efficacy and safety of | |
| | | polatuzumab vedotin | |
| | | combined with | |
| | | immunochemotherapy | |
| | | in patients with | |
| | | relapse/refractory | |
| | | diffuse large B cell | |
| | | lymphoma]. | |
| | | 2021 Terui Y, Rai S, I . . . | |
| | | A phase 2 study of | |
| | | polatuzumab vedotin + | |
| | | bendamustine + | |
| | | rituximab in | |
| | | relapsed/refractory | |
| | | diffuse large B-cell | |
| | | lymphoma. | |
| | | 2021 Dimou M, | |
| | | Papageor . . . REAL-LIFE | |
| | | EXPERIENCE WITH THE | |
| | | COMBINATION OF | |
| | | POLATUZUMAB | |
| | | VEDOTIN, RITUXIMAB | |
| | | AND BENDAMUSTINE | |
| | | IN AGGRESSIVE B-CELL | |
| | | LYMPHOMAS. | |
| | | 2020 Smith S D, | |
| | | Lopedot . . . | |
| | | Polatuzumab Vedotin | |
| | | for | |
| | | Relapsed/Refractory | |
| | | Aggressive B-cell | |
| | | Lymphoma: A | |
| | | Multicenter Post- | |
| | | marketing Analysis. | |
| | | 2020 Lu D, Lu T, Shi R . . . | |
| | | Application of a Two- | |
| | | Analyte Integrated | |
| | | Population | |
| | | Pharmacokinetic | |
| | | Model to Evaluate the | |
| | | Impact of Intrinsic and | |
| | | Extrinsic Factors on the | |
| | | Pharmacokinetics of | |
| | | Polatuzumab Vedotin | |
| | | in Patients with Non- | |
| | | Hodgkin Lymphoma. | |
| | | 2020 Kinoshita T, | |
| | | Hata . . . Safety and | |
| | | pharmacokinetics of | |
| | | polatuzumab vedotin in | |
| | | Japanese patients with | |
| | | relapsed/refractory B- | |
| | | cell non-Hodgkin | |
| | | lymphoma: a phase 1 | |
| | | dose-escalation study. | |
| | | 2020 Shi R, Lu T, Ku G . . . | |
| | | Asian race and origin | |
| | | have no clinically | |
| | | meaningful effects on | |
| | | polatuzumab vedotin | |
| | | pharmacokinetics in | |
| | | patients with | |
| | | relapsed/refractory B- | |
| | | cell non-Hodgkin | |
| | | lymphoma. | |
| | | 2020 Lu T, Gibiansky | |
| | | L . . . Exposure-safety | |
| | | and exposure-efficacy | |
| | | analyses of | |
| | | polatuzumab vedotin in | |
| | | patients with relapsed | |
| | | or refractory diffuse | |
| | | large B-cell lymphoma. | |
| | | 2020 Walji M, | |
| | | Assouline S An | |
| | | evaluation of | |
| | | polatuzumab vedotin | |
| | | for the treatment of | |
| | | patients with diffuse | |
| | | large B-cell lymphoma. | |
| | | 2020 Bourbon E, Salles | |
| | | G Polatuzumab | |
| | | vedotin: an | |
| | | investigational anti- | |
| | | CD79b antibody drug | |
| | | conjugate for the | |
| | | treatment of diffuse | |
| | | large B-cell lymphoma. | |
| | | 2020 Malecek M K, | |
| | | Watki . . . Polatuzumab | |
| | | vedotin for the | |
| | | treatment of adults | |
| | | with relapsed or | |
| | | refractory diffuse large | |
| | | B-cell lymphoma. | |
| | | 2020 Shemesh C S, | |
| | | Agarw . . . | |
| | | Pharmacokinetics of | |
| | | polatuzumab vedotin in | |
| | | combination with R/G- | |
| | | CHP in patients with B- | |
| | | cell non-Hodgkin | |
| | | lymphoma. | |
| | | 2019 Lu D, Lu T, Gibia . . . | |
| | | Integrated Two-Analyte | |
| | | Population | |
| | | Pharmacokinetic | |
| | | Model of Polatuzumab | |
| | | Vedotin in Patients | |
| | | with Non-Hodgkin | |
| | | Lymphoma. | |
| | | 2019 Sehn L H, Herrera . . . | |
| | | Polatuzumab | |
| | | Vedotin in Relapsed or | |
| | | Refractory Diffuse | |
| | | Large B-Cell | |
| | | Lymphoma. | |
| | | 2019 Deeks E D | |
| | | Polatuzumab Vedotin: | |
| | | First Global Approval. | |
| | | 2019 Li D, Lee D, | |
| | | Dere . . . Evaluation and | |
| | | use of an anti- | |
| | | cynomolgus monkey | |
| | | CD79b surrogate | |
| | | antibody-drug | |
| | | conjugate to enable | |
| | | clinical development of | |
| | | polatuzumab vedotin. | |
| | | 2019 Polatuzumab | |
| | | Vedotin Approved for | |
| | | DLBCL. | |
| | | 2019 Morschhauser F, | |
| | | F . . . Polatuzumab | |
| | | vedotin or | |
| | | pinatuzumab vedotin | |
| | | plus rituximab in | |
| | | patients with relapsed | |
| | | or refractory non- | |
| | | Hodgkin lymphoma: | |
| | | final results from a | |
| | | phase 2 randomised | |
| | | study (ROMULUS). | |
| | | 2017 Lu D, Gillespie | |
| | | W . . . Time-to-Event | |
| | | Analysis of | |
| | | Polatuzumab Vedotin- | |
| | | Induced Peripheral | |
| | | Neuropathy to Assist in | |
| | | the Comparison of | |
| | | Clinical Dosing | |
| | | Regimens. | |
| | | 2016 Fuh F K, Looney | |
| | | C, . . . Anti-CD22 and | |
| | | anti-CD79b antibody- | |
| | | drug conjugates | |
| | | preferentially target | |
| | | proliferating B cells. | |
| | | 2015 An Anti-CD79B | |
| | | Antibody-Drug | |
| | | Conjugate Is Active in | |
| | | Non-Hodgkin | |
| | | Lymphoma. | |
| | | 2015 Pfeifer M, Zheng . . . | |
| | | Anti-CD22 and anti- | |
| | | CD79B antibody drug | |
| | | conjugates are active in | |
| | | different molecular | |
| | | diffuse large B-cell | |
| | | lymphoma subtypes. | |
| | | 2014 Lu D, Jin J Y, Gir . . . | |
| | | Semi-mechanistic | |
| | | Multiple-Analyte | |
| | | Pharmacokinetic | |
| | | Model for an Antibody- | |
| | | Drug-Conjugate in | |
| | | Cynomolgus Monkeys. | |
| | | 2009 Zheng B, Fuji | |
| | | RN, . . . In vivo effects of | |
| | | targeting CD79b with | |
| | | antibodies and | |
| | | antibody-drug | |
| | | conjugates. | |
| | | 2009 Dornan D, | |
| | | Bennett . . . Therapeutic | |
| | | potential of an anti- | |
| | | CD79b antibody-drug | |
| | | conjugate, anti-CD79b- | |
| | | vc-MMAE, for the | |
| | | treatment of non- | |
| | | Hodgkin lymphoma. | |
| | | 2007 Polson A G, Yu | |
| | | S F, . . . Antibody-drug | |
| | | conjugates targeted to | |
| | | CD79 for the treatment | |
| | | of non-Hodgkin | |
| | | lymphoma. | |
| | | AACR 2016 Antibody | |
| | | drug conjugates (anti- | |
| | | CD79b-vc-MMAE, | |
| | | Polatuzumab Vedotin) | |
| | | exhibit enhanced cell | |
| | | death targeted to | |
| | | CD79b+ Burkitt | |
| | | lymphoma (BL) and | |
| | | primary mediastinal | |
| | | large B-cell lymphoma | |
| | | (PMBL) Aradhana | |
| | | Awasthi . . . | |
| | | AACR 2018 Enhanced | |
| | | in vitro/in vivo | |
| | | cytotoxicity against | |
| | | Burkitt | |
| | | lymphoma/primary | |
| | | mediastinal large B cell | |
| | | lymphoma by | |
| | | polatuzumab vedotin | |
| | | (hu- anti-CD79b-vc- | |
| | | MMAE, PV) alone or in | |
| | | combination with | |
| | | obinutuzumab A. A. | |
| | | Tiwari, D. . . . | |
| | | ASCO 2014 Preliminary | |
| | | results of a phase II | |
| | | randomized study | |
| | | (ROMULUS) of | |
| | | polatuzumab vedotin | |
| | | (PoV) or pinatuzumab | |
| | | vedotin (PiV) plus | |
| | | rituximab (RTX) in | |
| | | patients (Pts) with | |
| | | relapsed/refractory | |
| | | (R/R) non-Hodgkin | |
| | | lymphoma (NHL) | |
| | | Franck Morschhaus . . . | |
| | | ASCO 2016 A phase I | |
| | | pharmacokinetic and | |
| | | safety study of | |
| | | polatuzumab vedotin in | |
| | | Japanese patients with | |
| | | relapsed/refractory b- | |
| | | cell non-Hodgkin | |
| | | lymphoma: A | |
| | | comparison with non- | |
| | | Japanese DCS4968g | |
| | | study Kiyohiko | |
| | | Hatake, . . . | |
| | | ASH 2010 Targeted | |
| | | Depletion of B-Cell | |
| | | Subsets by Anti-CD22 | |
| | | and Anti- | |
| | | CD79bAntibody Drug | |
| | | Conjugates: | |
| | | Illumination of the | |
| | | Mechanism of Action | |
| | | through | |
| | | Pharmacodynamic | |
| | | Biomarkers Franklin | |
| | | Fuh*, Ca . . . | |
| Tuojie Biotech patent | 2021 WO2022022508 | | Tuojie Biotech |
| anti-CD79B | REN, Wenming, LIA . . . | | |
| | ANTI-CD79B | | |
| | ANTIBODY-DRUG | | |
| | CONJUGATE, AND | | |
| | PREPARATION | | |
| | METHOD THEREFOR | | |
| | AND | | |
| | PHARMACEUTICAL USE | | |
| | THEREOF | | |
| | 2020 WO2020156439 | | |
| | YANG, Cuiqing, TA . . . | | |
| | ANTI-CD79B | | |
| | ANTIBODY, ANTIGEN- | | |
| | BINDING FRAGMENT | | |
| | THEREOF, AND | | |
| | PHARMACEUTICAL USE | | |
| | THEREOF | | |
| | 2020 US20220162304 | | |
| | Yang, Cuiqing; Ta . . . | | |
| | ANTI-CD79B | | |
| | ANTIBODY, ANTIGEN- | | |
| | BINDING FRAGMENT | | |
| | THEREOF, AND | | |
| | PHARMACEUTICAL USE | | |
| | THEREOF | | |
| U.Texas patent anti- | 2021 WO2021222944 | | U.Texas |
| CD79b | CHU, Fuliang, NEE . . . | | |
| | ANTI-CD79B | | |
| | ANTIBODIES AND | | |
| | CHIMERIC ANTIGEN | | |
| | RECEPTORS AND | | |
| | METHODS OF USE | | |
| | THEREOF | | |
| | 2019 WO2020092467 | | |
| | CHU, Fuliang, NEE . . . | | |
| | ANTI-CD79B | | |
| | ANTIBODIES AND | | |
| | CHIMERIC ANTIGEN | | |
| | RECEPTORS AND | | |
| | METHODS OF USE | | |
| | THEREOF | | |
| | 2019 US20210317209 | | |
| | Chu, Fuliang; Nee . . . | | |
| | ANTI-CD79B | | |
| | ANTIBODIES AND | | |
| | CHIMERIC ANTIGEN | | |
| | RECEPTORS AND | | |
| | METHODS OF USE | | |
| | THEREOF | | |
| UCB patent anti-CD79b | 2016 WO2017009474 | | UCB |
| | FINNEY, Helene Ma . . . | | |
| | ANTIBODY MOLECULES | | |
| | WHICH BIND CD79 | | |
| | 2016 US20180201678 | | |
| | FINNEY, Helene Ma . . . | | |
| | ANTIBODY MOLECULES | | |
| | WHICH BIND CD79 | | |
| | 2016 U.S. Pat. No. 10,618,957 | | |
| | Finney, Helene Ma . . . | | |
| | Antibody molecules | | |
| | which bind CD79 | | |
| IBI38D9-L | | 2022 Wang J, Li C, He . . . | Innovent |
| | | Characterization of | |
| | | anti-CD79b/CD3 | |
| | | bispecific antibody, a | |
| | | potential therapy for B | |
| | | cell malignancies. | |
| Janssen patent anti- | 2022 US20220306738 | | Janssen Biotech |
| CD22/CD79B | Bednar, Kyle J.; . . . | | |
| | ANTIBODY TARGETING | | |
| | CD22 AND CD79B | | |
| | 2022 WO2022201052 | | |
| | BEDNAR, Kyle, KUM . . . | | |
| | ANTIBODY TARGETING | | |
| | CD22 AND CD79B | | |
| Mass. General Hosp. | 2019 WO2020124021 | | Mass. General Hosp. |
| patent anti-CD19/ | MAUS, Marcela, V. | | |
| CD79b CAR | CHIMERIC ANTIGEN | | |
| | RECEPTORS | | |
| | TARGETING CD79B | | |
| | AND CD19 | | |
| MGD010 | 2017 WO2017214096 | 2021 NCT05087628 | MacroGenics |
| | CHEN, Wei, MOORE, . . . | Phase 2 PRV-3279-2a | Provention Bio Takeda |
| | METHODS FOR THE | Trial in Systemic Lupus | |
| | USE OF CD32B X | 2019 NCT03955666 | |
| | CD79B-BINDING | Phase 1 A Phase 1b | |
| | MOLECULES IN THE | Study to Evaluate the | |
| | TREATMENT OF | Safety, Tolerability, | |
| | INFLAMMATORY | Pharmacokinetics, | |
| | DISEASES AND | Pharmacodynamics, | |
| | DISORDERS | and Immunogenicity of | |
| | 2017 US20190322741 | PRV-3279 in Healthy | |
| | Chen, Wei; Moore, . . . | Subjects | |
| | Methods for the Use of | 2015 NCT02376036 | |
| | CD32B × CD79B- | Phase 1 Phase 1 Study | |
| | Binding Molecules in | of MGD010 in Healthy | |
| | the Treatment of | Subjects | |
| | Inflammatory Diseases | | |
| | and Disorders | | |
| | 2014 WO2015021089 | | |
| | JOHNSON, Leslie, . . . BI- | | |
| | SPECIFIC | | |
| | MONOVALENT FC | | |
| | DIABODIES THAT ARE | | |
| | CAPABLE OF BINDING | | |
| | CD32B AND CD79B | | |
| | AND USES THEREOF | | |
| Nepenthe patent | 2021 US20210355214 | | Nepenthe |
| anti-CD79 | Larrick, James; Y . . . Anti- | | |
| | CD79 Antibodies and | | |
| | Their Uses | | |
| | 2019 US20200109198 | | |
| | Larrick, James; Y . . . Anti- | | |
| | CD79 Antibodies and | | |
| | Their Uses | | |
| | 2019 WO2020072705 | | |
| | LARRICK, James, Y . . . | | |
| | ANTI-CD79 | | |
| | ANTIBODIES AND | | |
| | THEIR USES | | |
| | 2019 U.S. Pat. No. 11,078,276 | | |
| | Larrick, James (S . . . Anti- | | |
| | CD79 antibodies and | | |
| | their uses | | |
| UCB patent anti- | 2016 WO2017009474 | | UCB |
| CD22/CD79b | FINNEY, Helene Ma . . . | | |
| | ANTIBODY MOLECULES | | |
| | WHICH BIND CD79 | | |
| | 2016 WO2017009476 | | |
| | FINNEY, Helene Ma . . . | | |
| | ANTIBODY MOLECULES | | |
| | WHICH BIND CD22 | | |
| | 2015 WO2016009030 | | |
| | FINNEY, Helene, M . . . | | |
| | MOLECULES WITH | | |
| | SPECIFICITY FOR CD79 | | |
| | AND CD22 | | |
| UCB patent anti- | 2021 WO2022079199 | | UCB |
| CD45/CD79b | RAPECKI, Stephen . . . | | |
| | BINDING MOLECULES | | |
| | THAT MULTIMERISE | | |
| | CD45 | | |
| | 2016 WO2017009474 | | |
| | FINNEY, Helene Ma . . . | | |
| | ANTIBODY MOLECULES | | |
| | WHICH BIND CD79 | | |
| | 2016 WO2017009473 | | |
| | FINNEY, Helene Ma . . . | | |
| | ANTIBODY MOLECULES | | |
| | WHICH BIND CD45 | | |
| | 2016 US20180237521 | | |
| | FINNEY, Helene Ma . . . | | |
| | ANTIBODY MOLECULES | | |
| | WHICH BIND CD45 | | |
| | 2015 WO2016009029 | | |
| | FINNEY, Helene Ma . . . | | |
| | MOLECULES WITH | | |
| | SPECIFICITY FOR CD45 | | |
| | AND CD79 | | |
| Janssen patent anti- | 2022 WO2022200443 | | Janssen Biotech |
| CD79b/CD20/CD3 | GANESAN, Rajkumar . . . | | |
| | TRISPECIFIC ANTIBODY | | |
| | TARGETING CD79b, | | |
| | CD20, AND CD3 | | |
| | 2022 US20220315663 | | |
| | GANESAN, Rajkumar . . . | | |
| | TRISPECIFIC ANTIBODY | | |
| | TARGETING CD79b, | | |
| | CD20, AND CD3 | | |

i. HER2

In some embodiments, a polynucleotide provided herein comprises a nucleotide sequence encoding a HER2 CAR, a variable domain of a HER2 CAR, or a set of CDRs (HCDR 1, 2, and 3 and LCDR 1, 2, and 3) that encodes a HER2 CAR as set forth in TABLE 28 below or a variable domain of a HER2 CAR or a set of CDRs (HCDR 1, 2, and 3 and LCDR 1, 2, and 3) therefrom. In some embodiments, a polynucleotide provided herein comprises a nucleotide sequence encoding a HER2 CAR, a variable domain of a HER2 CAR, or a set of CDRs (HCDR 1, 2, and 3 and LCDR 1, 2, and 3) that having at least 80% (e.g., at least 80%, at least 85%, at least 90%, at least 95%, at least 96%, at least 97%, at least 98%, at least 99%, or 100% identical) to a HER2 CAR as set forth in TABLE 28 below or a variable domain of a HER2 CAR or a set of CDRs (HCDR 1, 2, and 3 and LCDR 1, 2, and 3) therefrom, respectively.

TABLE 28
Exemplary HER2 antigen binding domains

| Antibody Name | Company |
|---|---|
| 3SBio anti-Her2 | 3SBio |
| A166 | Klus Pharma Sichuan Kelun Pharma |
| Abbott patent anti-Her2 | Abbott |
| AbGenomics patent anti-Her2 | AltruBio |
| AbMax patent anti-Her2 | AbMax |
| Academia Sinica patent anti-Her2 | Academia Sinica |
| ADCT-502 | ADC Therapeutics Medimmune |
| AK-HER2 | Anhui Anke Bio |
| Alper patent anti-Her2 | Alper |
| ALT-P7 | 3SBio Alteogen |
| Amgen patent anti-Her2 | Amgen |
| AMX-818 | Amunix |
| anbenitamab | Roche |
| ARX788 | Ambrx Zhejiang Medicine |
| AstraZeneca patent anti-Her2 | AstraZeneca Medimmune |
| B002 | Shanghai Pharma Holdings |
| B003 | Shanghai Pharma Holdings |
| BAT8001 | Bio-Thera Solutions |
| BAY2701439 | Bayer |
| Baylor Coll. Med. anti-Her2 CAR | Baylor Coll. Med. |
| BB-1701 | Bliss Bio |
| BCD-022 | Biocad |
| BCD-147 | Biocad |
| BDC-1001 | Bolt Bio Stanford |
| BioMab patent anti-Her2 | BioMab |
| BSI-001 | Biosion |
| BT2111 | Bioasis |
| CAT-01-106 | Catalent |
| Chia Tai Tianqing Pharma pertuzumab biosimilar | Chia Tai Tianqing Pharma |
| Chinese Acad. Sci. Pertuzumab-MCC-DM1 | Chinese Acad. Sci. |
| Chinese PLA Gen. Hosp. anti-Her2 CAR | Chinese PLA Gen. Hosp. |
| Chong Kun Dang patent anti-Her2 | Chong Kun Dang |
| Chugai patent anti-Her2 | Chugai |
| City of Hope patent anti-Her2 CAR | City of Hope |
| CMAB302 | Shanghai CP Guojian |
| CMI Cuba 5G4 | CMI Cuba |
| coprelotamab | Genor Shanghai Escugen |
| COVA208 | Covagen |
| CRX02 | Curaxys |
| Denali patent anti-Her2 | Denali |
| disitamab vedotin | RemeGen |
| DMB-3111 | Meiji Seika |
| DP303c | CSPC Pharma |
| DRL_TZ | Dr. Reddy's |
| Duke U. anti-Her2 | Duke U. |
| DXL702 | InNexus |
| Erbicin | Biotecnol |
| Fourth Mil. Med. U patent anti-Her2 | Fourth Military Med. U. |
| FS-1502 | Fosun Pharma |
| FS102 | BMS f-star |
| gancotamab | Merrimack |
| Genentech anti-Her2 Domain 1 | Genentech |
| Glenmark patent anti-Her2 | Glenmark/Ichnos |
| GNR-027 | IBC Generium |
| GQ1001 | GeneQuantum |
| Green Cross patent anti-Her2 CAR | Green Cross |
| H2Mab-139 | Tohoku U. |
| HD201 | Prestige BioPharma |
| Hebreastin | Aryogen |
| Hersintuzumab | Tehran U. Med. Sci. |
| HLX02 | Shanghai Henlius |
| HLX11 | Shanghai Henlius |
| HLX22 | Abclon Alligator Shanghai Henlius |
| HS627 | Zheijang Hisun |
| HuMax-Her2 | Genmab |
| IBI patent anti-Her2 | IBI |
| IBI354 | Innovent |
| IGN001 | ImmunGene |
| ImmuneOnco patent anti-Her2 fusion | ImmuneOnco |
| INSERM patent anti-Her2 | INSERM |
| Inst. Basic Med. Sci. MIL5_scFv | Beijing Inst. Basic Med. Sci. |
| Institut Curie patent anti-Her2 | CNRS Institut Curie |
| Isfahan U. Pertuzumab ScFv | Isfahan U. Med. Sci. |
| JHL1188 | JHL Biotech |
| JHL1199 | JHL Biotech |
| Jiangsu Simcere patent anti-HER2 | Jiangsu Simcere |
| KHK patent trastuzumab variant | KHK |
| King's College anti-Her2 CAR | King's College |
| KN026 | Alphamab |
| LCB14-0110 | LegoChem |
| Lupin pertuzumab biosimilar | Lupin |
| LZM005 | Livzon |
| m860 | NIH |
| MAB270 | MAB Discovery |
| margetuximab | MacroGenics Zai Lab |
| MBS301 | Beijing Mabworks |
| Med. Bio. Labs patent anti-Her2 | Med. Bio. Labs |
| Medarex patent anti-Her2/neu | Medarex |
| MI130004 | PharmaMar |
| MRG002 | Shanghai Miracogen |
| NBE-Therapeutics trastuzumab Maytansine ADC | NBE-Therapeutics |
| NeuCeptin | NeuClone |
| NJH395 | Novartis |
| NM-02 | Suzhou Nanomab |
| NRC Canada anti-Her2 | NRC Canada |
| NRC Canada camelid anti-Her2 | NRC Canada |
| NRC Canada patent anti-Her2 Tikhomirov | NRC Canada |
| Olivia Newton-John Cancer Res. Inst. patent anti- | Olivia Newton-John Cancer Res. Inst. |
| Her2 | |
| Ono patent anti-Her2 | Ono |
| ONS-1050 | Oncobiologics |
| ORM-5029 | Orum |
| P013 | Mashhad U. |
| Palleon patent anti-Her2 | Palleon |
| Pasteur Inst. Iran anti-Her2 | Pasteur Inst. Iran |
| pertuzumab | Genentech |
| pertuzumab zuvotolimod | Silverback |
| PF-05280014 | Pfizer |
| PF-06804103 | Pfizer |
| PF-06888667 | Pfizer |
| Pierre Fabre patent anti-Her2 ADC | Pierre Fabre |
| QL1209 | Qilu Pharma Sound Biologics |
| QLHER2 | |
| RC Bio patent anti-Her2 | RC Bio |
| Redwood patent anti-Her2 | Redwood |
| Regeneron patent anti-Her2 biparatopic | Regeneron |
| RG6148 | Genentech |
| RG6194 | Genentech |
| SB3 | Merck (MSD) Samsung Bioepis |
| Scripps patent anti-Her2 | Scripps |
| Second Mil. Med. U. trastuzumab emtansine | Second Military Med Univ |
| biosimilar | |
| Second Military Med Univ anti-Her2 H2-18 | Second Military Med Univ |
| Second Military Med Univ anti-Her2 TPL | Second Military Med Univ |
| Shahid Beheshti U. Med. Sci. anti-Her2 | Shahid Beheshti U. Med. Sci. |
| Shanghai Bao Pharma patent anti-Her2 | Shanghai Bao Pharma |
| Shanghai Jiao Tong U. anti-Her2 bispecific | Shanghai Jiao Tong U. |
| Shanghai PAE patent anti-Her2 | Shanghai PAE |
| Shenzhen Bindebiotech patent anti-Her2 CAR | Shenzhen Bindebiotech |
| Shiraz U. Med. Sci. Pertuzumab biosimilar | Shiraz U. Med. Sci. |
| SIBP-01 | Shanghai Inst. Bio. Products |
| Singapore ASTR patent anti-Her2 | Singapore ASTR |
| Spirogen trastuzumab ADC | Spirogen |
| Sun Yat-Sen U. anti-Her2 | Sun Yat-Sen U. |
| Sunshine Guojian patent anti-Her2 | Sunshine Guojian Pharma |
| Suzhou Kangju Bio patent anti-Her2 | Suzhou Kangju Bio |
| SYD983 | Synthon |
| Symphogen patent anti-Her2 | Symphogen |
| Systimmune patent anti-Her2 | Systimmune |
| TA4415V | Tehran U. Med. Sci. |
| Tarbiat Modares U. 1F2 | Tarbiat Modares U. |
| timigutuzumab | Glycotope |
| TQB2930 | Chia Tai Tianqing Pharma |
| trastuzumab | Genentech Roche |
| trastuzumab beta | |
| trastuzumab deruxtecan | AstraZeneca Daiichi Sankyo |
| trastuzumab duocarmazine | Synthon |
| trastuzumab emtansine | Genentech ImmunoGen PDL Roche |
| trastuzumab meditecan | MediBoston |
| trastuzumab-anns | Allergan Amgen |
| trastuzumab-dkst | Biocon Mylan |
| Trastuzumab-dolaflexin | Adimab Mersana |
| trastuzumab-MMAE | Antitope |
| trastuzumab-pkrb | Celltrion |
| trastuzumab-TCMC | NCI |
| Trubion patent anti-ErbB2 | Trubion Wyeth |
| TX05 | Tanvex |
| U of ST China patent anti-Her2 | U. of S.T. China |
| U. California patent anti-Her2 | U. California |
| U. Napoli anti-ErbB2 | U. Napoli |
| U. Zurich patent anti-Her2 | Univ. Zurich |
| UB-921 | United Biopharma |
| VB7-756 | Viventia |
| Vrije Universiteit Brussel patent anti-Her2 | Vrije Universiteit Brussel |
| XMT-1522 | Adimab Mersana Takeda |
| XMT-2056 | GSK Mersana |
| Xuanzhu Bio patent anti-Her2 | Xuanzhu Bio |
| Yeda patent anti-Her2/neu | Yeda R&D |
| YM Biosciences patent anti-Her2 | NRC Canada YM BioSciences |
| zanidatamab | Jazz Pharma Zymeworks |
| zanidatamab zovodotin | Medimmune |
| ZRC-3256 | Cadila Zydus |
| ZV0201 | Zova Bio |
| ZW33 | Zymeworks |
| ZW49 | Zymeworks |
| 1G5D2 | Tehran U. Med. Sci. |
| ABP-100 | Abpro Sloan-Kettering |
| Ampsource patent anti-Her2/CD3 | Ampsource |
| Anhui U. anti-Her2/EGFR | Anhui Anke Bio |
| Bioatla patent anti-Her2/CD3 | BioAtla |
| Biocad anti-Her2/Her3 | Biocad |
| Biomunex patent anti-Her2/EGFR | Biomunex INSERM |
| BiOneCure patent anti-Her2/Trop-2 | BiOneCure |
| Chinese Acad. Sci. anti-Her2/PD-1 | Chinese Acad. Sci. |
| Dartsbio anti-Her2/PD-L1 | Dartsbio Shanghai Mabstone |
| ertumaxomab | Fresenius |
| Eutilex patent anti-4-1BB/Her2 | Eutilex |
| Fox Chase anti-Her2/Her3 | Fox Chase |
| GBR1302 | Glenmark/Ichnos Harbour Biomed Ltd. |
| Genentech patent anti-Her2/VEGF | Genentech |
| Genmab anti-Her2/CD63 | Genmab |
| Genmab patent anti-Her2/CD3 | Genmab |
| German CRC anti-NKG2D/Her2 | German CRC |
| Guangzhou Excelmab patent anti-CD3/Her2 | Guangzhou Excelmab |
| HLX31 | Henlix |
| IBI315 | Beijing Hanmi Innovent |
| IMM2902 | ImmuneOnco |
| Kanda Bio patent anti-CTLA-4/Her2 | Kanda Bio |
| M802 | Wuhan YZY Biopharma |
| Merus patent anti-EGFR/Her2 | Merus |
| MM-111 | Merrimack |
| Novartis patent anti-Her1/Her2 | Novartis |
| PRS-343 | Pieris |
| Regeneron anti-Her2/APLP2 | Regeneron |
| Regeneron anti-Her2/PRLR ADC | Regeneron |
| Roche Glycart patent anti-Her2/cMet | Glycart |
| Roche patent anti-CD28/Her2 | Roche |
| Roche patent anti-CD3/Her2 | Roche |
| Roche patent anti-Her2/Her3 | Roche |
| Roche patent anti-Her2/Tfr trispecific | Roche |
| runimotamab | Genentech |
| Samsung patent anti-cMet/Her2 | Samsung |
| Samsung patent anti-EGFR/Her2 | Samsung |
| Shanghai Jiao Tong U. anti-EGFR/Her2 | Shanghai Jiao Tong U. |
| Shengbiao Med. Equip. patent anti-Her2/VEGF | Shengbiao Med. Equip. |
| Sichuan University anti-CD3/Her2 | Sichuan University |
| SSGJ-705 | Sunshine Guojian Pharma |
| Sun Yat-Sen U. anti-Her2/CD16 | Sun Yat-Sen U. |
| Sun Yat-Sen U. anti-Her2/CD3 | Sun Yat-Sen U. |
| Sunshine Guojian patent anti-PD-L1/HER2 | Sunshine Guojian Pharma |
| Tavotek patent anti-HER2/VEGF | Tavotek |
| Tokyo Med. U. anti-Her2/Fc gamma RIIIb | Tokyo Med. U. |
| U. Hong Kong patent anti-IGF-1R/Her2 | U. Hong Kong |
| U. North Carolina patent anti-EGFR/HER2 CAR | UNC |
| U. Texas patent anti-Her2/Her3 | U. Texas |
| U. Virginia anti-Her2/CD3 | U. Virginia |
| Uppsala U. anti-EGFR and HER2 | Uppsala U. |
| X-Body patent anti-Her2/PDGFRB | X-Body |
| YH32367 | ABL Bio Yuhan |
| zenocutuzumab | Merus |
| Zensun patent anti-Her2/Her3 | Zensun |
| Zymeworks patent anti-Her2/Her3 | Zymeworks |
| Dragonfly patent anti-NKG2D/CD16/HER2 | Dragonfly |
| SAR443216 | Sanofi |
| Sunshine Guojian patent anti-PD-1/Her2/LAG-3 | Sunshine Guojian Pharma |
| Sym013 | Symphogen |
| CRTB6 | Second Military Med Univ |
| FL518 | Second Military Med Univ |

j. IL-13R Alpha2

In some embodiments, a polynucleotide provided herein comprises a nucleotide sequence encoding a IL-13R ALPHA2 CAR, a variable domain of a IL-13R ALPHA2 CAR, or a set of CDRs (HCDR 1, 2, and 3 and LCDR 1, 2, and 3) that encodes a IL-13R ALPHA2 CAR as set forth in TABLE 29 below or a variable domain of a IL-13R ALPHA2 CAR or a set of CDRs (HCDR 1, 2, and 3 and LCDR 1, 2, and 3) therefrom. In some embodiments, a polynucleotide provided herein comprises a nucleotide sequence encoding a IL-13R ALPHA2 CAR, a variable domain of a IL-13R ALPHA2 CAR, or a set of CDRs (HCDR 1, 2, and 3 and LCDR 1, 2, and 3) that having at least 80% (e.g., at least 80%, at least 85%, at least 90%, at least 95%, at least 96%, at least 97%, at least 98%, at least 99%, or 100% identical) to a IL-13R ALPHA2 CAR as set forth in TABLE 29 below or a variable domain of a IL-13R ALPHA2 CAR or a set of CDRs (HCDR 1, 2, and 3 and LCDR 1, 2, and 3) therefrom, respectively.

TABLE 29
Exemplary IL-13R ALPHA2 antigen binding domains

| Antibody Name | Patents | Publications | Company |
|---|---|---|---|
| ADC Therapeutics | 2021 US20220033508 | | ADC Therapeutics |
| patent anti-IL-13R | 2021 WO2022023522 | | |
| alpha 2 | | | |
| Capital Med. U. anti-IL- | | 2022 Xu C, Bai Y, An Z . . . | Capital Med. U. |
| 13Ralpha2 CAR | | IL-13Rα2 humanized | |
| | | scFv-based CAR-T cells | |
| | | exhibit therapeutic | |
| | | activity against | |
| | | glioblastoma. | |
| Carsgen patent anti-IL- | 2018 WO2018149358 | | Carsgen |
| 13RA2 | 2018 US20190359723 | | |
| | 2018 U.S. Pat. No. 11,530,270 | | |
| City of Hope IL-13Ra2 | 2021 WO2022109498 | 2019 NCT04003649 | City of Hope |
| CAR | | Phase 1 IL13Ralpha2- | |
| | | Targeted Chimeric | |
| | | Antigen Receptor (CAR) | |
| | | T Cells With or Without | |
| | | Nivolumab and | |
| | | Ipilimumab in Treating | |
| | | Patients With | |
| | | Recurrent or Refractory | |
| | | Glioblastoma | |
| CSIC patent anti-IL- | 2022 WO2022207727 | | CSIC |
| 13RA2 | | | |
| Elicera patent anti-IL- | 2021 WO2021230792 | | Elicera |
| 13Ralpha2 | | | |
| Moffitt Cancer Center | 2018 WO2018156711 | | Moffitt Cancer Center |
| patent anti-IL-13RA2 | | | |
| CAR | | | |
| Pfizer patent anti-IL- | 2013 WO2014072888 | AACR 2015 Impact of | Pfizer |
| 13Rα2 | 2013 US20150266962 | conjugation site on | |
| | 2013 U.S. Pat. No. 9,828,428 | pharmacokinetics and | |
| | | off-target toxicity of | |
| | | site-specific antibody | |
| | | drug conjugates | |
| | | Dangshe Ma, Fang . . . | |
| Qilu patent anti-IL-13R | 2022 WO2022174808 | | Qilu Pharma Sound |
| alpha 2 | | | Biologics |
| U. Chicago patent anti- | 2021 WO2021207770 | | U. Chicago |
| IL-13Ra2 CAR | 2016 WO2016123143 | | |
| | 2016 WO2016123142 | | |
| Wake Forest U. patent | 2020 US20200299394 | | Wake Forest U. |
| anti-IL-13RA2 | 2017 US20180155437 | | |
| | 2017 U.S. Pat. No. 10,676,529 | | |
| | 2014 US20160039938 | | |
| | 2014 U.S. Pat. No. 9,868,788 | | |
| | 2014 WO2014152361 | | |
| Wistar Inst. patent | 2022 WO2022198027 | | Wistar |
| anti-IL-13R alpha 2/X | | | |

k. MUC1

In some embodiments, a polynucleotide provided herein comprises a nucleotide sequence encoding a MUC1 CAR, a variable domain of a MUC1 CAR, or a set of CDRs (HCDR 1, 2, and 3 and LCDR 1, 2, and 3) that encodes a MUC1 CAR as set forth in TABLE 30 below or a variable domain of a MUC1 CAR or a set of CDRs (HCDR 1, 2, and 3 and LCDR 1, 2, and 3) therefrom. In some embodiments, a polynucleotide provided herein comprises a nucleotide sequence encoding a MUC1 CAR, a variable domain of a MUC1 CAR, or a set of CDRs (HCDR 1, 2, and 3 and LCDR 1, 2, and 3) that having at least 80% (e.g., at least 80%, at least 85%, at least 90%, at least 95%, at least 96%, at least 97%, at least 98%, at least 99%, or 100% identical) to a MUC1 CAR as set forth in TABLE 30 below or a variable domain of a MUC1 CAR or a set of CDRs (HCDR 1, 2, and 3 and LCDR 1, 2, and 3) therefrom, respectively.

TABLE 30
Exemplary MUC1 antigen binding domains

| Antibody Name | Patents | Publications | Company |
|---|---|---|---|
| ADC Therapeutics | 2015 US20170267778 | | ADC Therapeutics |
| patent anti-Tn-MUC1 | VAN BERKEL, Patri . . . | | Medimmune |
| | HUMANIZED ANTI-TN- | | |
| | MUC1 ANTIBODIES AND | | |
| | THEIR CONJUGATES | | |
| | 2015 U.S. Pat. No. 10,017,580 Van | | |
| | Berkel, Patri . . . | | |
| | Humanized anti-Tn- | | |
| | MUC1 antibodies and | | |
| | their conjugates | | |
| AR20.5 | 2019 US20190322760 | 2017 Movahedin M, | Oncoquest Quest |
| | Madiyalakan, Ragu . . . | Broo . . . Glycosylation of | |
| | TUMOR ANTIGEN | MUC1 influences the | |
| | SPECIFIC ANTIBODIES | binding of a | |
| | AND TLR3 STIMULATION | therapeutic antibody | |
| | TO ENHANCE THE | by altering the | |
| | PERFORMANCE OF | conformational | |
| | CHECKPOINT | equilibrium of the | |
| | INTERFERENCE THERAPY | antigen. | |
| | OF CANCER | 2004 de Bono JS, Rha | |
| | 2017 US20200206347 | S . . . Phase I trial of a | |
| | Madiyalakan, Ragu . . . | murine antibody to | |
| | METHOD OF INDIRECT | MUC1 in patients with | |
| | IMMUNIZATION OF | metastatic cancer: | |
| | HUMAN OVARIAN | evidence for the | |
| | CANCER PATIENTS | activation of humoral | |
| | THROUGH SELECTION OF | and cellular antitumor | |
| | XENOGENEIC | immunity. | |
| | IMMUNOGLOBULIN FC | 2001 Qi W, Schultes | |
| | PORTIONS | BC . . . Characterization | |
| | 2015 US20170226221 | of an anti-MUC1 | |
| | Madiyalakan, Ragu . . . | monoclonal antibody | |
| | TUMOR ANTIGEN | with potential as a | |
| | SPECIFIC ANTIBODIES | cancer vaccine. | |
| | AND TLR3 STIMULATION | | |
| | TO ENHANCE THE | | |
| | PERFORMANCE OF | | |
| | CHECKPOINT | | |
| | INTERFERENCE THERAPY | | |
| | OF CANCER | | |
| | 2015 U.S. Pat. No. 10,392,444 | | |
| | Madiyalakan, Ragu . . . | | |
| | Tumor antigen specific | | |
| | antibodies and TLR3 | | |
| | stimulation to enhance | | |
| | the performance of | | |
| | checkpoint interference | | |
| | therapy of cancer | | |
| | 2015 WO2016019472 | | |
| | MADIYALAKAN, Ragu . . . | | |
| | TUMOR ANTIGEN | | |
| | SPECIFIC ANTIBODIES | | |
| | AND TLR3 STIMULATION | | |
| | TO ENHANCE THE | | |
| | PERFORMANCE OF | | |
| | CHECKPOINT | | |
| | INTERFERENCE THERAPY | | |
| | OF CANCER | | |
| Astellas patent anti- | 2020 WO2020145227 | | Astellas |
| MUC1 | AKAIWA, Michinori . . . | | |
| | COMPLEX COMPRISING | | |
| | LIGAND, SPACER, | | |
| | PEPTIDE LINKER, AND | | |
| | BIOMOLECULE | | |
| | 2019 US20200171174 | | |
| | MORINAKA, Akifumi . . . | | |
| | NOVEL ANTI-HUMAN | | |
| | MUC1 ANTIBODY FAB | | |
| | FRAGMENT | | |
| | 2019 US20200261603 | | |
| | MORINAKA, Akifumi . . . | | |
| | NOVEL ANTI-HUMAN | | |
| | MUC1 ANTIBODY FAB | | |
| | FRAGMENT | | |
| | 2019 US20190269804 | | |
| | Morinaka, Akifumi . . . | | |
| | NOVEL ANTI-HUMAN | | |
| | MUC1 ANTIBODY FAB | | |
| | FRAGMENT | | |
| | 2019 WO2019221269 | | |
| | ASANO, Toru, SANO . . . | | |
| | COMPLEX HAVING ANTI- | | |
| | HUMAN MUC1 | | |
| | ANTIBODY Fab | | |
| | FRAGMENT, PEPTIDE | | |
| | LINKER AND/OR LIGAND | | |
| | 2019 U.S. Pat. No. 10,507,251 | | |
| | Morinaka, Akifumi . . . | | |
| | Anti-human MUC1 | | |
| | antibody fab fragment | | |
| | 2019 US20210340276 | | |
| | ASANO, Toru; SANO . . . | | |
| | COMPLEX HAVING ANTI- | | |
| | HUMAN MUC1 | | |
| | ANTIBODY FAB | | |
| | FRAGMENT, PEPTIDE | | |
| | LINKER AND/OR LIGAND | | |
| | 2017 WO2018092885 | | |
| | MORINAKA, Akifumi . . . | | |
| | NOVEL ANTI-HUMAN | | |
| | MUC1 ANTIBODY Fab | | |
| | FRAGMENT | | |
| | 2017 US20190307906 | | |
| | MORINAKA, Akifumi . . . | | |
| | NOVEL ANTI-HUMAN | | |
| | MUC1 ANTIBODY FAB | | |
| | FRAGMENT | | |
| | 2017 U.S. Pat. No. 10,517,966 | | |
| | Morinaka, Akifumi . . . | | |
| | Anti-human MUC1 | | |
| | antibody Fab fragment | | |
| BioArdis patent anti- | 2021 WO2022133074 | | BioArdis |
| MUC1 | HAERIZADEH, Farza . . . | | |
| | MUC1 BINDING | | |
| | MOLECULES AND USES | | |
| | THEREOF | | |
| BTH1704 | | 2014 NCT02132403 | Biothera U. Illinois |
| | | Phase 1 (PM-01) | |
| | | IMPRIME PGG ® With | |
| | | BTH1704 and | |
| | | Gemcitabine for | |
| | | Advanced Pancreatic | |
| | | Cancer | |
| Cancer Res. Tech. | 2018 US20180334507 | | Cancer Research |
| patent anti-MUC1 | Clausen, Henrik; . . . | | Technology |
| | GENERATION OF A | | |
| | CANCER-SPECIFIC | | |
| | IMMUNE RESPONSE | | |
| | TOWARD MUC1 AND | | |
| | CANCER SPECIFIC MUC1 | | |
| | ANTIBODIES | | |
| | 2018 U.S. Pat. No. 10,919,973 | | |
| | Clausen, Henrik ( . . . | | |
| | Generation of a cancer- | | |
| | specific immune | | |
| | response toward MUC1 | | |
| | and cancer specific | | |
| | MUC1 antibodies | | |
| | 2016 WO2016166305 | | |
| | VAN BERKEL, Patri . . . | | |
| | SITE-SPECIFIC | | |
| | ANTIBODY-DRUG | | |
| | CONJUGATES | | |
| | 2016 WO2016166341 | | |
| | VAN BERKEL, Patri . . . | | |
| | SITE-SPECIFIC | | |
| | ANTIBODY-DRUG | | |
| | CONJUGATES | | |
| | 2016 WO2016166300 | | |
| | VAN BERKEL, Patri . . . | | |
| | SITE-SPECIFIC | | |
| | ANTIBODY-DRUG | | |
| | CONJUGATES | | |
| | 2015 WO2015159076 | | |
| | VAN BERKEL, Patri . . . | | |
| | HUMANIZED ANTI-TN- | | |
| | MUC1 ANTIBODIES AND | | |
| | THEIR CONJUGATES | | |
| Cell Signaling patent | 2014 WO2015009740 | | Bluefin Biomed. Cell |
| anti-MUC1 | SCHOEN, Robert E . . . | | Signaling U. |
| | ANTI-MUCIN 1 BINDING | | Pittsburgh |
| | AGENTS AND USES | | |
| | THEREOF | | |
| | 2014 US20160145343 | | |
| | Schoen, Robert E . . . | | |
| | ANTI-MUCIN 1 BINDING | | |
| | AGENTS AND USES | | |
| | THEREOF | | |
| | 2014 U.S. Pat. No. 10,208,125 | | |
| | Schoen, Robert E . . . Anti- | | |
| | mucin 1 binding agents | | |
| | and uses thereof | | |
| clivatuzumab | 2017 US20170137534 | 2014 NCT01956812 | Immunomedics |
| tetraxetan | Goldenberg, David . . . | Phase 3 Phase 3 Trial of | |
| | Anti-Pancreatic Cancer | 90Y-Clivatuzumab | |
| | Antibodies | Tetraxetan & | |
| | 2016 US20170035908 | Gemcitabine vs | |
| | Gold, David V.; G . . . | Placebo & Gemcitabine | |
| | Detection of Early-Stage | in Metastatic | |
| | Pancreatic | Pancreatic Cancer | |
| | Adenocarcinoma | 2012 NCT01510561 | |
| | 2015 US20160090413 | Phase 1 A Study of | |
| | Liu, Donglin; Gol . . . ANTI- | Fractionated 90Y- | |
| | MUCIN ANTIBODIES FOR | hPAM4 Plus | |
| | EARLY DETECTION AND | Gemcitabine in | |
| | TREATMENT OF | Pancreatic Cancer | |
| | PANCREATIC CANCER | Patients Receiving at | |
| | 2015 U.S. Pat. No. 9,513,293 Gold, | Least 2 Prior Therapies. | |
| | David V.; G . . . Detection | 2008 NCT00603863 | |
| | of early-stage pancreatic | Phase 1/Phase 2 Safety | |
| | adenocarcinoma | and Efficacy Study of | |
| | 2015 US20150320872 | Different Doses of 90Y- | |
| | Gold, David V.; G . . . Anti- | hPAM4 Combined With | |
| | Mucin Antibodies for | Gemcitabine in | |
| | Early Detection and | Pancreatic Cancer | |
| | Treatment of Pancreatic | 2007 NCT00364364 | |
| | Cancer | Phase 1 Safety Study to | |
| | 2015 US20150165076 | Determine the | |
| | Liu, Donglin; Gol . . . ANTI- | Appropriate Dose of | |
| | MUCIN ANTIBODIES FOR | Antibody Against | |
| | EARLY DETECTION AND | Tumor Cells to Best | |
| | TREATMENT OF | Target Patients With | |
| | PANCREATIC CANCER | Pancreatic Cancer. | |
| | 2015 U.S. Pat. No. 9,238,084 Liu, | 2004 NCT00597129 | |
| | Donglin; Gol . . . Anti- | Phase 1/Phase 2 Safety | |
| | mucin antibodies for | and Efficacy Study of | |
| | early detection and | 90Y-hPAM4 at | |
| | treatment of pancreatic | Different Doses | |
| | cancer | 2015 Picozzi VJ, | |
| | 2015 U.S. Pat. No. 9,272,057 | Raman . . . (90)Y- | |
| | Govindan, Serengu . . . | clivatuzumab | |
| | Combining | tetraxetan with or | |
| | radioimmunotherapy | without low-dose | |
| | and antibody-drug | gemcitabine: A phase | |
| | conjugates for improved | Ib study in patients | |
| | cancer therapy | with metastatic | |
| | 2015 US20150132768 | pancreatic cancer after | |
| | Goldenberg, David . . . | two or more prior | |
| | Anti-Pancreatic Cancer | therapies. | |
| | Antibodies | 2013 Gold DV, | |
| | 2015 U.S. Pat. No. 9,599,619 | Newsome . . . Mapping | |
| | Goldenberg, David . . . | PAM4 (clivatuzumab), | |
| | Anti-pancreatic cancer | a monoclonal antibody | |
| | antibodies | in clinical trials for | |
| | 2014 US20140377173 | early detection and | |
| | Gold, David V.; G . . . Anti- | therapy of pancreatic | |
| | Mucin Antibodies for | ductal | |
| | Early Detection and | adenocarcinoma, to | |
| | Treatment of Pancreatic | MUC5AC mucin. | |
| | Cancer | 2012 Ocean AJ, | |
| | 2014 U.S. Pat. No. 9,089,618 Gold, | Penning . . . Fractionated | |
| | David V.; G . . . Anti-mucin | radioimmunotherapy | |
| | antibodies for early | with (90) Y- | |
| | detection and treatment | clivatuzumab | |
| | of pancreatic cancer | tetraxetan and low- | |
| | 2014 US20140294722 | dose gemcitabine is | |
| | Goldenberg, David . . . | active in advanced | |
| | Anti-Pancreatic Cancer | pancreatic cancer: A | |
| | Antibodies | phase 1 trial. | |
| | 2014 U.S. Pat. No. 8,974,784 | 2011 Gulec SA, Cohen | |
| | Goldenberg, David . . . | S . . . Treatment of | |
| | Anti-pancreatic cancer | advanced pancreatic | |
| | antibodies | carcinoma with 90Y- | |
| | 2014 US20140227179 | clivatuzumab | |
| | Liu, Donglin; Gol . . . ANTI- | tetraxetan: A Phase I | |
| | MUCIN ANTIBODIES FOR | single-dose escalation | |
| | EARLY DETECTION AND | trial. | |
| | TREATMENT OF | 2011 Sharkey RM, | |
| | PANCREATIC CANCER | Karac . . . Combination | |
| | 2014 U.S. Pat. No. 9,005,613 Liu, | radioimmunotherapy | |
| | Donglin; Gol . . . Anti- | and | |
| | mucin antibodies for | chemoimmunotherapy | |
| | early detection and | involving different or | |
| | treatment of pancreatic | the same targets | |
| | cancer | improves therapy of | |
| | 2013 US20140112864 | human pancreatic | |
| | Gold, David V.; G . . . Anti- | carcinoma xenograft | |
| | Mucin Antibodies for | models. | |
| | Early Detection and | 2009 Karacay H, | |
| | Treatment of Pancreatic | Sharke . . . Pretargeted | |
| | Cancer | radioimmunotherapy | |
| | 2013 U.S. Pat. No. 8,795,662 Gold, | of pancreatic cancer | |
| | David V.; G . . . Anti-mucin | xenografts: TF10-90Y- | |
| | antibodies for early | IMP-288 alone and | |
| | detection and treatment | combined with | |
| | of pancreatic cancer | gemcitabine. | |
| | 2013 US20140037543 | | |
| | Goldenberg, David . . . | | |
| | Anti-Pancreatic Cancer | | |
| | Antibodies | | |
| | 2013 U.S. Pat. No. 8,821,868 | | |
| | Goldenberg, David . . . | | |
| | Anti-pancreatic cancer | | |
| | antibodies | | |
| | 2012 WO2012112443 | | |
| | GOLD, David V., G . . . | | |
| | ANTI-MUCIN | | |
| | ANTIBODIES FOR EARLY | | |
| | DETECTION AND | | |
| | TREATMENT OF | | |
| | PANCREATIC CANCER | | |
| | 2012 U.S. Pat. No. 8,574,854 Gold, | | |
| | David V.; G . . . Anti-mucin | | |
| | antibodies for early | | |
| | detection and treatment | | |
| | of pancreatic cancer | | |
| | 2011 U.S. Pat. No. 9,238,081 Gold, | | |
| | David V.; G . . . Detection | | |
| | of early-stage pancreatic | | |
| | adenocarcinoma | | |
| | 2010 WO2011068845 | | |
| | GOVINDAN, Serengu . . . | | |
| | COMBINING | | |
| | RADIOIMMUNOTHERAPY | | |
| | AND ANTIBODY-DRUG | | |
| | CONJUGATES FOR | | |
| | IMPROVED CANCER | | |
| | THERAPY | | |
| | 2010 U.S. Pat. No. 8,435,529 | | |
| | Govindan, Serengu . . . | | |
| | Combining | | |
| | radioimmunotherapy | | |
| | and antibody-drug | | |
| | conjugates for improved | | |
| | cancer therapy | | |
| | 2009 WO2010017500 | | |
| | GOLDENBERG, David . . . | | |
| | ANTI-PANCREATIC | | |
| | CANCER ANTIBODIES | | |
| | 2009 U.S. Pat. No. 8,491,896 | | |
| | Goldenberg, David . . . | | |
| | Anti-pancreatic cancer | | |
| | antibodies | | |
| | 2007 US20080050311 | | |
| | Goldenberg, David . . . | | |
| | Monoclonal Antibody | | |
| | hPAM4 | | |
| | 2003 WO2003106497 | | |
| | GOLD, David, V.; . . . | | |
| | MONOCLONAL | | |
| | ANTIBODY PAM4 AND | | |
| | ITS USE FOR DIAGNOSIS | | |
| | AND THERAPY OF | | |
| | PANCREATIC CANCER | | |
| | 2003 WO2003106495 | | |
| | GOLDENBERG, David . . . | | |
| | HUMANIZED | | |
| | MONOCLONAL | | |
| | ANTIBOBY HPAM4 | | |
| | 2003 U.S. Pat. No. 7,282,567 | | |
| | Goldenberg, David . . . | | |
| | Monoclonal antibody | | |
| | hPAM4 | | |
| CRC patent anti-MUC1 | 2016 U.S. Pat. No. 10,017,576 | | CRC |
| | Clausen, Henrik ( . . . | | |
| | Generation of a cancer- | | |
| | specific immune | | |
| | response toward MUC1 | | |
| | and cancer specific | | |
| | MUC1 antibodies | | |
| | 2014 U.S. Pat. No. 9,359,436 | | |
| | Clausen, Henrik; . . . | | |
| | Generation of a cancer- | | |
| | specific immune | | |
| | response toward MUC1 | | |
| | and cancer specific | | |
| | MUC1 antibodies | | |
| | 2007 U.S. Pat. No. 8,440,798 | | |
| | Clausen, Henrik; . . . | | |
| | Generation of a cancer- | | |
| | specific immune | | |
| | response toward MUC1 | | |
| | and cancer specific | | |
| | MUC1 antibodies | | |
| | 2007 WO2008040362 | | |
| | CLAUSEN, Henrik, . . . | | |
| | GENERATION OF A | | |
| | CANCER-SPECIFIC | | |
| | IMMUNE RESPONSE | | |
| | TOWARD MUC1 AND | | |
| | CANCER SPECIFIC MUC1 | | |
| | ANTIBODIES | | |
| | 2001 WO2002044217 | | |
| | LO, Benny, Kwan, . . . | | |
| | HUMANISED | | |
| | ANTIBODIES AND USES | | |
| | THEREOF | | |
| CTM01 | 2007 WO2008038024 | 2014 Lozano N, Al- | Celltech |
| | BAKER, Terry, Sew . . . | Ahma . . . Monoclonal | |
| | ALTERED ANTIBODIES | antibody-targeted | |
| | 1992 WO1993006231 | PEGylated liposome- | |
| | ADAIR, John, Robe . . . | ICG encapsulating | |
| | ANTI-HUMAN MILK FAT | doxorubicin as a | |
| | GLOBULE HUMANISED | potential theranostic | |
| | ANTIBODIES | agent. | |
| | | 1997 Banfield MJ, | |
| | | King . . . VL:VH domain | |
| | | rotations in engineered | |
| | | antibodies: crystal | |
| | | structures of the Fab | |
| | | fragments from two | |
| | | murine antitumor | |
| | | antibodies and their | |
| | | engineered human | |
| | | constructs. | |
| Daiichi Sankyo patent | 2019 WO2019219891 | | Daiichi Sankyo |
| anti-MUC1 | RÜHMANN, Johanna, . . . | | |
| | ANTI-MUC1 ANTIBODY - | | |
| | DRUG CONJUGATE | | |
| | 2019 US20210187118 | | |
| | GELLERT, Johanna; . . . | | |
| | ANTI-MUC1 ANTIBODY- | | |
| | DRUG CONJUGATE | | |
| DMB5F3 | 2021 WO2021186427 | 2020 Pichinuk E, | Tel-Aviv U. |
| | RUBINSTEIN, Danie . . . | Chali . . . In vivo anti- | |
| | ANTI- MUC1-SEA | MUC1+ tumor activity | |
| | ANTIBODIES | and sequences of high- | |
| | 2011 U.S. Pat. No. 8,648,172 | affinity anti-MUC1-SEA | |
| | Rubinstein, Danie . . . Anti- | antibodies. | |
| | MUC1 α/β antibodies | 2012 Pichinuk E, | |
| | | Benha . . . Antibody | |
| | | targeting of cell-bound | |
| | | MUC1 SEA domain kills | |
| | | tumor cells. | |
| Dyax patent anti- | 2003 WO2003089451 | | Dyax |
| MUC1 | HOOGENBOOM, Hendr . . . | | |
| | ANTIBODIES SPECIFIC | | |
| | FOR MUCIN | | |
| | POLYPEPTIDE | | |
| | 2001 WO2001075110 | | |
| | HOOGENBOOM, Hendr . . . | | |
| | MUCIN-1 SPECIFIC | | |
| | BINDING MEMBERS AND | | |
| | METHODS OF USE | | |
| | THEREOF | | |
| gatipotuzumab | 2019 WO2019219889 | 2017 NCT03360734 | Glycotope |
| | RÜHMANN, Johanna, . . . | Phase 1 Combination | |
| | ANTI-MUC1 ANTIBODY | of Gatipotuzumab and | |
| | 2019 US20210221910 | Tomuzotuximab in | |
| | GELLERT, Johanna; . . . | Patients With Solid | |
| | ANTI-MUC1 ANTIBODY | Tumors | |
| | 2019 WO2019166617 | 2013 NCT01899599 | |
| | RÜHMANN, Johanna, . . . | Phase 2 PankoMab- | |
| | FUSION PROTEIN | GEX ™ Versus Placebo | |
| | CONSTRUCTS | as Maintenance | |
| | COMPRISING AN ANTI- | Therapy in Advanced | |
| | MUC1 ANTIBODY AND | Ovarian Cancer | |
| | IL-15 | 2009 NCT01222624 | |
| | 2019 US20210107961 | Phase 1 PankoMab- | |
| | GELLERT, Johanna; . . . | GEX ™: Dose Escalation | |
| | Fusion Protein | Study | |
| | Constructs Comprising | 2022 Ochsenreither S, . . . | |
| | an Anti-MUC1 Antibody | Erratum to ‘Safety | |
| | and IL-15 | and preliminary activity | |
| | 2018 US20200148785 | results of the GATTO | |
| | Kehler, Patrik; G . . . PD-L1 | study, a phase Ib study | |
| | AND TA-MUC1 | combining the anti-TA- | |
| | ANTIBODIES | MUC1 antibody | |
| | 2018 US20200131275 | Gatipotuzumab with | |
| | GOLETZ, Steffen; . . . | the anti-EGFR | |
| | MULTISPECIFIC | Tomuzotuximab in | |
| | ANTIBODY CONSTRUCTS | patients with | |
| | BINDING TO MUC1 AND | refractory solid | |
| | CD3 | tumors’: [ESMO Open | |
| | 2018 WO2018138113 | Volume 7, Issue 2, April | |
| | GOLETZ, Steffen, . . . | 2022, 100447]. | |
| | ANTI-CANCER | 2022 Ochsenreither S, . . . | |
| | TREATMENTS WITH AN | Safety and | |
| | ANTI-MUC1 ANTIBODY | preliminary activity | |
| | AND AN ERBB INHIBITOR | results of the GATTO | |
| | 2018 US20190343953 | study, a phase Ib study | |
| | GOLETZ, Steffen; . . . | combining the anti-TA- | |
| | ANTI-CANCER | MUC1 antibody | |
| | TREATMENTS WITH AN | gatipotuzumab with | |
| | ANTI-MUC1 ANTIBODY | the anti-EGFR | |
| | AND AN ERBB INHIBITOR | tomuzotuximab in | |
| | 2017 WO2017162733 | patients with | |
| | GOLETZ, Steffen, . . . IGA | refractory solid | |
| | ANTIBODIES WITH | tumors. | |
| | ENHANCED STABILITY | 2021 Ledermann JA, | |
| | 2011 U.S. Pat. No. 9,359,439 Goletz, | Zur . . . Maintenance | |
| | Steffen; . . . Fab- | therapy of patients | |
| | glycosylated antibodies | with recurrent | |
| | 2010 WO2011012309 | epithelial ovarian | |
| | GOLETZ, Steffen; . . . | carcinoma with the | |
| | MUC1 ANTIBODIES | anti-tumor-associated- | |
| | 2010 US20120128676 | mucin-1 antibody | |
| | Goletz, Steffen; . . . MUC1 | gatipotuzumab: results | |
| | ANTIBODIES | from a double-blind, | |
| | 2010 U.S. Pat. No. 9,217,038 Goletz, | placebo-controlled, | |
| | Steffen; . . . MUC1 | randomized, phase II | |
| | antibodies | study. | |
| | | 2018 Heublein S, | |
| | | Fries . . . TA-MUC1 as | |
| | | detected by the fully | |
| | | humanized, | |
| | | therapeutic antibody | |
| | | Gatipotzumab predicts | |
| | | poor prognosis in | |
| | | cervical cancer. | |
| | | 2016 Fiedler W, | |
| | | DeDoss . . . A phase I | |
| | | study of PankoMab- | |
| | | GEX, a humanised | |
| | | glyco-optimised | |
| | | monoclonal antibody | |
| | | to a novel tumour- | |
| | | specific MUC1 | |
| | | glycopeptide epitope in | |
| | | patients with advanced | |
| | | carcinomas. | |
| | | 2015 Heublein S, Mayr . . . | |
| | | Immunoreactivity of | |
| | | the fully humanized | |
| | | therapeutic antibody | |
| | | PankoMab-GEX(TM) is | |
| | | an independent | |
| | | prognostic marker for | |
| | | breast cancer patients. | |
| | | 2013 Dian D, Lenhard | |
| | | M . . . Staining of MUC1 | |
| | | in ovarian cancer | |
| | | tissues with | |
| | | PankoMab-GEX ™ | |
| | | detecting the tumour- | |
| | | associated epitope, TA- | |
| | | MUC1, as compared to | |
| | | antibodies HMFG-1 | |
| | | and 115D8. | |
| | | ASCO 2018 The GATTO | |
| | | study: A phase I of the | |
| | | anti-MUC1 | |
| | | Gatipotuzumab (GAT) | |
| | | in combination with | |
| | | the anti-EGFR | |
| | | Tomuzotuximab (TO) in | |
| | | patients with EGFR | |
| | | positive solid tumors. | |
| | | Sebastian Ochsenr . . . | |
| Genus Oncology | 2021 US20220049014 | 2020 Detappe A, | Dana-Farber Genus |
| patent anti-MUC1 | Kufe, Donald W.; . . . | Mathie . . . Anti-MUC1-C | Oncology LLC |
| | ANTIBODIES AGAINST | Antibody-Conjugated | |
| | THE MUC1- | Nanoparticles | |
| | C/EXTRACELLULAR | Potentiate the Efficacy | |
| | DOMAIN (MUC1-C/ECD) | of Fractionated | |
| | 2021 WO2022016190 | Radiation Therapy. | |
| | KHARBANDA, Surend . . . | 2018 Panchamoorthy | |
| | ANTIBODIES AGAINST | G, . . . Targeting the | |
| | THE MUC1- | human MUC1-C | |
| | C/EXTRACELLULAR | oncoprotein with an | |
| | DOMAIN (MUC1-C/ECD) | antibody-drug | |
| | 2020 WO2020252472 | conjugate. | |
| | MARASCO, Wayne A . . . | 2011 Panchamoorthy | |
| | ANTIBODIES AGAINST | G, . . . A monoclonal | |
| | MUC1 AND METHODS | antibody against the | |
| | OF USE THEREOF | oncogenic mucin 1 | |
| | 2020 US20220289863 | cytoplasmic domain. | |
| | Marasco, Wayne A . . . | | |
| | ANTIBODIES AGAINST | | |
| | MUC1 AND METHODS | | |
| | OF USE THEREOF | | |
| | 2018 US20180312605 | | |
| | KUFE, Donald W.; . . . | | |
| | ANTIBODIES AGAINST | | |
| | THE MUC1- | | |
| | C/EXTRACELLULAR | | |
| | DOMAIN (MUC1-C/ECD) | | |
| | 2018 U.S. Pat. No. 11,136,410 Kufe, | | |
| | Donald W. ( . . . Antibodies | | |
| | against the MUC1- | | |
| | C/extracellular domain | | |
| | (MUC1-C/ECD) | | |
| | 2017 US20180036441 | | |
| | KUFE, Donald W.; . . . | | |
| | ANTIBODIES AGAINST | | |
| | THE MUC1- | | |
| | C/EXTRACELLULAR | | |
| | DOMAIN (MUC1-C/ECD) | | |
| | 2017 U.S. Pat. No. 10,617,773 Kufe, | | |
| | Donald W. ( . . . Antibodies | | |
| | against the MUC1- | | |
| | C/extracellular domain | | |
| | (MUC1-C/ECD) | | |
| | 2015 WO2015116753 | | |
| | KUFE, Donald, W., . . . | | |
| | ANTIBODIES AGAINST | | |
| | THE MUC1- | | |
| | C/EXTRACELLULAR | | |
| | DOMAIN (MUC1-C/ECD) | | |
| | 2015 US20160340442 | | |
| | KUFE, Donald W.; . . . | | |
| | ANTIBODIES AGAINST | | |
| | THE MUC1- | | |
| | C/EXTRACELLULAR | | |
| | DOMAIN (MUC1-C/ECD) | | |
| | 2015 U.S. Pat. No. 10,059,775 Kufe, | | |
| | Donald W. ( . . . Antibodies | | |
| | against the MUC1- | | |
| | C/extracellular domain | | |
| | (MUC1-C/ECD) | | |
| | 2012 US20130039974 | | |
| | Kufe, Donald W.; . . . | | |
| | ANTI-MUC1 ANTIBODIES | | |
| | FOR CANCER | | |
| | DIAGNOSTICS | | |
| | 2012 WO2013023162 | | |
| | KUFE, Donald, W., . . . | | |
| | ANTI-MUC1 ANTIBODIES | | |
| | FOR CANCER | | |
| | DIAGNOSTICS | | |
| Glaxo patent anti-Tn- | 2021 WO2021191819 | | GSK |
| Muc1 | BHINDER, Tejinder . . . | | |
| | ANTI-TN-MUC1 | | |
| | CHIMERIC ANTIGEN | | |
| | RECEPTORS | | |
| Go Therapeutics | 2021 US20220089772 | | Go Thera |
| patent anti-glyco- | WHITE, Thayer; ANTI- | | |
| MUC1 | GLYCO-MUC1 | | |
| | ANTIBODIES AND THEIR | | |
| | USES | | |
| | 2019 WO2020006449 | | |
| | WHITE, Thayer, WA . . . | | |
| | ANTI-GLYCO-MUC1 | | |
| | ANTIBODIES AND THEIR | | |
| | USES | | |
| | 2019 US20210269552 | | |
| | White, Thayer; WA . . . | | |
| | ANTI-GLYCO-MUC1 | | |
| | ANTIBODIES AND THEIR | | |
| | USES | | |
| | 2018 US20190119400 | | |
| | WHITE, Thayer; ANTI- | | |
| | GLYCO-MUC1 | | |
| | ANTIBODIES AND THEIR | | |
| | USES | | |
| | 2018 U.S. Pat. No. 11,161,911 | | |
| | White, Thayer (Bo . . . | | |
| | Anti-glyco-MUC1 | | |
| | antibodies and their uses | | |
| | 2017 WO2019083506 | | |
| | WHITE, Thayer ANTI- | | |
| | GLYCO-MUC1 | | |
| | ANTIBODIES AND THEIR | | |
| | USES | | |
| Guangzhou Anjie | | 2018 NCT03633773 | Guangzhou Anjie |
| Biomed. Tech. anti- | | Phase 1/Phase 2 Safety | Biomed. Tech. |
| Muc1 CAR | | and Efficacy Evaluation | |
| | | of MUC-1 CART in the | |
| | | Treatment of | |
| | | Intrahepatic | |
| | | Cholangiocarcinoma | |
| | | 2018 NCT03525782 | |
| | | Phase 1/Phase 2 Anti- | |
| | | MUC1 CAR T Cells and | |
| | | PD-1 Knockout | |
| | | Engineered T Cells for | |
| | | NSCLC | |
| Immunomedics TF10 | 2012 U.S. Pat. No. 8,652,484 | | Immunomedics |
| | McBride, William . . . | | |
| | Delivery system for | | |
| | cytotoxic drugs by | | |
| | bispecific antibody | | |
| | pretargeting | | |
| | 2010 U.S. Pat. No. 8,435,539 | | |
| | McBride, William . . . | | |
| | Delivery system for | | |
| | cytotoxic drugs by | | |
| | bispecific antibody | | |
| | pretargeting | | |
| Kagoshima U. patent | 2011 US20130045490 | | Kagoshima U. |
| anti-Muc1 | Yonezawa, Suguru; | | |
| | ANTIBODY AGAINST | | |
| | MUCIN 1 (MUC1) | | |
| | PROTEIN AND USE OF | | |
| | SAME | | |
| | 2011 U.S. Pat. No. 8,951,526 | | |
| | Yonezawa, Suguru | | |
| | Antibody against mucin | | |
| | 1 (MUC1) protein and | | |
| | use of same | | |
| | 2011 WO2011099566 | | |
| | YONEZAWA Suguru | | |
| | ANTIBODY AGAINST | | |
| | MUCIN 1 (MUC1) | | |
| | PROTEIN AND USE OF | | |
| | SAME | | |
| LFB patent anti-MUC-1 | 2009 WO2010006810 | | LFB Yumab |
| | BEHRENS, Christia . . . | | |
| | RECOMBINANT ANTI- | | |
| | MUC1 ANTIBODIES | | |
| | 2009 US20110318757 | | |
| | Behrens, Christia . . . | | |
| | RECOMBINANT ANTI- | | |
| | MUC1 ANTIBODIES | | |
| | 2009 U.S. Pat. No. 9,546,217 | | |
| | Behrens, Christia . . . | | |
| | Recombinant anti-MUC1 | | |
| | antibodies | | |
| Med. Chem. Pharma | 2015 WO2015166934 | | Med. Chem. Pharma |
| patent anti-MUC1 | NISHIMURA Shinich . . . | | |
| | ANTI-MUC1 ANTIBODY | | |
| | OR ANTIGEN-BINDING | | |
| | FRAGMENT OF SAME, | | |
| | AND USE THEREOF | | |
| | 2015 US20170198056 | | |
| | Nishimura, Shinic . . . Anti- | | |
| | MUC1 Antibody or | | |
| | Antigen-Binding | | |
| | Fragment Thereof and | | |
| | Uses Thereof | | |
| | 2015 U.S. Pat. No. 10,239,950 | | |
| | Nishimura, Shinic . . . Anti- | | |
| | MUC1 antibody or | | |
| | antigen-binding | | |
| | fragment thereof and | | |
| | uses thereof | | |
| Minerva patent anti- | 2021 WO2022027039 | | Minerva |
| MUC1 | BAMDAD, Cynthia, . . . | | |
| | ANTI-VARIABLE MUC1* | | |
| | ANTIBODIES AND USES | | |
| | THEREOF | | |
| | 2021 US20220184120 | | |
| | Bamdad, Cynthia; . . . | | |
| | ANTI-VARIABLE MUC1* | | |
| | ANTIBODIES AND USES | | |
| | THEREOF | | |
| | 2020 WO2020146902 | | |
| | BAMDAD, Cynthia ANTI- | | |
| | VARIABLE MUC1* | | |
| | ANTIBODIES AND USES | | |
| | THEREOF | | |
| | 2019 US20200239594 | | |
| | Bamdad, Cynthia; . . . | | |
| | MUC1* ANTIBODIES | | |
| | 2019 U.S. Pat. No. 11,560,435 | | |
| | Bamdad, Cynthia; . . . | | |
| | MUC1* antibodies | | |
| | 2019 WO2019165421 | | |
| | BAMDAD, Cynthia | | |
| | DIAGNOSTIC METHODS | | |
| | USING ANTI-MUC1* | | |
| | ANTIBODIES | | |
| | 2019 US20200407462 | | |
| | BAMDAD, Cynthia; | | |
| | DIAGNOSTIC METHODS | | |
| | USING ANTI-MUC1* | | |
| | ANTIBODIES | | |
| | 2018 WO2019104306 | | |
| | BAMDAD, Cynthia | | |
| | HUMANIZED ANTI- | | |
| | MUC1* ANTIBODIES | | |
| | AND DIRECT USE OF | | |
| | CLEAVAGE ENZYME | | |
| | 2018 US20200390870 | | |
| | BAMDAD, Cynthia; | | |
| | HUMANIZED ANTI- | | |
| | MUC1* ANTIBODIES | | |
| | AND DIRECT USE OF | | |
| | CLEAVAGE ENZYME | | |
| | 2017 WO2018071583 | | |
| | BAMDAD, Cynthia, . . . | | |
| | HUMANIZED ANTI- | | |
| | MUC1* ANTIBODIES | | |
| | AND USE OF CLEAVAGE | | |
| | ENZYME | | |
| | 2017 US20190290692 | | |
| | Bamdad, Cynthia; . . . | | |
| | HUMANIZED ANTI- | | |
| | MUC1* ANTIBODIES | | |
| | AND USE OF CLEAVAGE | | |
| | ENZYME | | |
| | 2016 US20170121406 | | |
| | BAMDAD, Cynthia; STEM | | |
| | CELL ENHANCING | | |
| | THERAPEUTICS | | |
| | 2016 WO2016130726 | | |
| | BAMDAD, Cynthia, . . . | | |
| | HUMANIZED ANTI- | | |
| | MUC1* ANTIBODIES | | |
| | 2016 US20180112007 | | |
| | BAMDAD, Cynthia; . . . | | |
| | HUMANIZED ANTI- | | |
| | MUC1* ANTIBODIES | | |
| | 2015 US20150299334 | | |
| | Bamdad, Cynthia; STEM | | |
| | CELL ENHANCING | | |
| | THERAPEUTICS | | |
| | 2015 U.S. Pat. No. 9,932,407 | | |
| | Bamdad, Cynthia Stem | | |
| | cell enhancing | | |
| | therapeutics | | |
| | 2009 WO2010042562 | | |
| | MAHANTA, Sanjeev; . . . | | |
| | MUC1* ANTIBODIES | | |
| | 2009 US20170204191 | | |
| | BAMDAD, Cynthia C . . . | | |
| | MUC1* ANTIBODIES | | |
| | 2009 US20180222998 | | |
| | BAMDAD, Cynthia C . . . | | |
| | MUC1* ANTIBODIES | | |
| | 2009 U.S. Pat. No. 10,421,819 | | |
| | Bamdad, Cynthia C . . . | | |
| | MUC1* antibodies | | |
| Nanocruise patent | 2017 WO2018132976 | | Nanocruise Pharma |
| anti-MUC1 | ZHOU, Dapeng, HWU . . . | | U. Texas |
| | MONOCLONAL AND | | |
| | HUMANIZED | | |
| | ANTIBODIES TO A | | |
| | CANCER GLYCOPEPTIDE | | |
| | 2017 US20200115466 | | |
| | ZHOU, Dapeng; HWU . . . | | |
| | MONOCLONAL AND | | |
| | HUMANIZED | | |
| | ANTIBODIES TO A | | |
| | CANCER GLYCOPEPTIDE | | |
| pemtumomab | | | Antisoma |
| Peptron patent anti- | 2020 US20210115153 | | Peptron Inc. |
| MUC1 | MOON, Kyung Duk; . . . | | |
| | ANTIBODY BINDING | | |
| | SPECIFICALLY TO MUC1 | | |
| | AND USE THEREOF | | |
| | 2018 WO2018174544 | | |
| | MOON, Kyung Duk, | | |
| | [symbol] ANTIBODY BINDING | | |
| | SPECIFICALLY TO MUC1 | | |
| | AND USE THEREOF | | |
| | 2018 | | |
| | US20200024361 | | |
| | MOON, Kyung Duk; . . . | | |
| | ANTIBODY BINDING | | |
| | SPECIFICALLY TO MUC1 | | |
| | AND USE THEREOF | | |
| | 2018 U.S. Pat. No. 11,472,887 | | |
| | Moon, Kyung Duk | | |
| | Antibody binding | | |
| | specifically to MUC1 and | | |
| | use thereof | | |
| PersonGen anti-MUC1 | | 2015 NCT02617134 | PersonGen |
| CAR | | Phase 1/Phase 2 CAR-T | |
| | | Cell Immunotherapy in | |
| | | MUC1 Positive Solid | |
| | | Tumor | |
| | | 2015 NCT02587689 | |
| | | Phase 1/Phase 2 Phase | |
| | | I/II Study of Anti- | |
| | | Mucin1 (MUC1) CAR T | |
| | | Cells for Patients With | |
| | | MUC1+ Advanced | |
| | | Refractory Solid Tumor | |
| Poseida patent anti- | 2020 WO2021127505 | | Poseida |
| MUC1 CAR | OSTERTAG, Eric M . . . | | |
| | ANTI-MUC1 | | |
| | COMPOSITIONS AND | | |
| | METHODS OF USE | | |
| | 2017 WO2018014039 | | |
| | OSTERTAG, Eric, S . . . | | |
| | CHIMERIC ANTIGEN | | |
| | RECEPTORS (CARS) | | |
| | SPECIFIC FOR MUC1 AND | | |
| | METHODS FOR THEIR | | |
| | USE | | |
| | 2017 WO2018014038 | | |
| | OSTERTAG, Eric, S . . . | | |
| | CHIMERIC ANTIGEN | | |
| | RECEPTORS AND | | |
| | METHODS FOR USE | | |
| R.P. Scherer Tech. | 2021 US20220033514 | | R.P. Scherer Tech. |
| patent anti-MUC1 | Rabuka, David; Dr . . . | | |
| | ANTIBODY SPECIFIC FOR | | |
| | MUCIN-1 AND METHODS | | |
| | OF USE THEREOF | | |
| | 2021 WO2022026809 | | |
| | RABUKA, David, DR . . . | | |
| | ANTIBODY SPECIFIC FOR | | |
| | MUCIN-1 AND METHODS | | |
| | OF USE THEREOF | | |
| R1549 | | | Antisoma Roche |
| Shanghai Cell Therapy | 2018 WO2019020087 | | Shanghai Cell Therapy |
| Res. Inst. patent anti- | QIAN, Qijun, [symbol] , . . . | | Res. Inst. |
| MUC1 CAR | CHIMERIC ANTIGEN | | |
| | RECEPTOR-MODIFIED T | | |
| | CELL TARGETING MUC1 | | |
| | AND USE THEREOF | | |
| Shionogi patent anti- | 2011 US20130045543 | | Shionogi |
| MUC1 | Nishimura, Shin-I . . . | | |
| | NOVEL MUC1 ANTIBODY | | |
| | 2011 U.S. Pat. No. 8,883,977 | | |
| | Nishimura, Shin-I . . . | | |
| | MUC1 antibody | | |
| | 2009 WO2010050528 | | |
| | NISHIMURA Shin-Ic . . . | | |
| | ANTI-MUC1 ANTIBODY | | |
| | 2009 US20120040375 | | |
| | Nishimura, Shin-I . . . ANTI- | | |
| | MUC1 ANTIBODY | | |
| | 2009 U.S. Pat. No. 8,722,856 | | |
| | Nishimura, Shin-I . . . Anti- | | |
| | MUC1 antibody | | |
| SNU patent anti-MUC1 | 2010 WO2011099684 | | SNU R&DB |
| | SEONG, Seung-Yong . . . | | |
| | SINGLE DOMAIN | | |
| | ANTIBODY AGAINST | | |
| | MUC1 | | |
| sontuzumab | 2006 US20090110632 | 2008 NCT00770354 | Antisoma Roche |
| | Young, Robert; Jo . . . | Phase 2 Phase II Study | |
| | BIOLOGICAL MATERIALS | of AS1402 Combined | |
| | AND USES THEREOF | With Letrozole to Treat | |
| | 2006 WO2007034210 | Breast Cancer | |
| | JONES, David; YOU . . . | 2004 NCT00096057 | |
| | DEGLYCOSYLATED ANTI- | Phase 1 Monoclonal | |
| | MUC-1 ANTIBODIES AND | Antibody HuHMFG1 in | |
| | USES THEREOF | Treating Women With | |
| | | Locally Advanced or | |
| | | Metastatic Breast | |
| | | Cancer | |
| | | 2003 NCT00066547 | |
| | | Phase 1/Phase 2 | |
| | | Monoclonal Antibody | |
| | | Therapy in Treating | |
| | | Women With Locally | |
| | | Advanced or | |
| | | Metastatic Breast | |
| | | Cancer Previously | |
| | | Treated With | |
| | | Combination | |
| | | Chemotherapy | |
| | | 2011 Ibrahim NK, | |
| | | Yariz . . . Randomized | |
| | | Phase II Trial of | |
| | | Letrozole plus Anti- | |
| | | MUC1 Antibody | |
| | | AS1402 in Hormone | |
| | | Receptor-positive | |
| | | Locally Advanced or | |
| | | Metastatic Breast | |
| | | Cancer. | |
| | | 2010 Royer B, Yin W, | |
| | | P . . . Population | |
| | | pharmacokinetics of | |
| | | the humanised | |
| | | monoclonal antibody, | |
| | | HuHMFG1 (AS1402), | |
| | | derived from a phase I | |
| | | study on breast cancer. | |
| | | 2007 Moreno M, | |
| | | Bontkes . . . High level of | |
| | | MUC1 in serum of | |
| | | ovarian and breast | |
| | | cancer patients inhibits | |
| | | huHMFG-1 dependent | |
| | | cell-mediated | |
| | | cytotoxicity (ADCC). | |
| SPmAb2.1 | 2017 US20190185558 | | Vaxil |
| | Carmon, Lior; KOV . . . | | |
| | ANTIBODIES DIRECTED | | |
| | AGAINST SIGNAL | | |
| | PEPTIDES, METHODS | | |
| | AND USES THEREOF | | |
| Stanford U. patent | 2019 US20200087413 | | Stanford |
| anti-MUC1 | Mollick, Joseph A . . . IgE | | |
| | Antibodies for the | | |
| | Inhibition of Tumor | | |
| | Metastasis | | |
| | 2017 US20170129965 | | |
| | Mollick, Joseph A . . . IgE | | |
| | ANTIBODIES FOR THE | | |
| | INHIBITION OF TUMOR | | |
| | METASTASIS | | |
| | 2017 U.S. Pat. No. 10,487,152 | | |
| | Mollick, Joseph A . . . IgE | | |
| | antibodies for the | | |
| | inhibition of tumor | | |
| | metastasis | | |
| | 2014 WO2014201212 | | |
| | MOLLICK, Joseph, . . . IgE | | |
| | ANTIBODIES FOR THE | | |
| | INHIBITION OF TUMOR | | |
| | METASTASIS | | |
| | 2013 US20140370001 | | |
| | Mollick, Joseph A . . . IgE | | |
| | ANTIBODIES FOR THE | | |
| | INHIBITION OF TUMOR | | |
| | METASTASIS | | |
| | 2013 U.S. Pat. No. 9,587,032 | | |
| | Mollick, Joseph A . . . IgE | | |
| | antibodies for the | | |
| | inhibition of tumor | | |
| | metastasis | | |
| TAB-004 | 2019 WO2021045728 | 2013 Curry JM, | UNC |
| | PINKU, Mukherjee | Thompso . . . The use of | |
| | TUMOR SPECIFIC | a novel MUC1 antibody | |
| | ANTIBODY CONJUGATES | to identify cancer stem | |
| | AND USES THEREFOR | cells and circulating | |
| | 2019 U.S. Pat. No. 11,554,181 | MUC1 in mice and | |
| | Mukherjee, Pinku; . . . | patients with | |
| | Tumor specific antibody | pancreatic cancer. | |
| | conjugates and uses | | |
| | therefor | | |
| | 2017 US20180043038 | | |
| | Mukherjee, Pinku; . . . | | |
| | TUMOR SPECIFIC | | |
| | ANTIBODY CONJUGATES | | |
| | AND USES THEREFOR | | |
| | 2017 WO2017120525 | | |
| | MUKHERJEE, Pinku | | |
| | COMPOSITIONS | | |
| | COMPRISING CHIMERIC | | |
| | ANTIGEN RECEPTORS, T | | |
| | CELLS COMPRISING THE | | |
| | SAME AND METHODS OF | | |
| | USING THE SAME | | |
| | 2016 US20160319031 | | |
| | Mukherjee, Pinku; | | |
| | TUMOR SPECIFIC | | |
| | ANTIBODIES AND USES | | |
| | THEREFOR | | |
| | 2015 US20160067358 | | |
| | Mukherjee, Pinku; . . . | | |
| | TUMOR SPECIFIC | | |
| | ANITBODY CONJUGATES | | |
| | AND USES THEREFOR | | |
| | 2013 US20140010759 | | |
| | Mukherjee, Pinku; | | |
| | TUMOR SPECIFIC | | |
| | ANITBODIES AND USES | | |
| | THEREFOR | | |
| | 2011 WO2012047317 | | |
| | MUKHERJEE, Pinku | | |
| | TUMOR SPECIFIC | | |
| | ANTIBODIES AND USES | | |
| | THEREFOR | | |
| | 2011 US20130336886 | | |
| | Mukherjee, Pinku; | | |
| | TUMOR SPECIFIC | | |
| | ANTIBODIES AND USES | | |
| | THEREFOR | | |
| | 2011 U.S. Pat. No. 9,090,698 | | |
| | Mukherjee, Pinku Tumor | | |
| | specific antibodies and | | |
| | uses therefor | | |
| Tehran U. Med. Sci anti- | | 2020 Rajabzadeh A, | Tehran U. Med. Sci. |
| MUC1 CAR | | Rah . . . A VHH-Based | |
| | | Anti-MUC1 Chimeric | |
| | | Antigen Receptor for | |
| | | Specific Retargeting of | |
| | | Human Primary T Cells | |
| | | to MUC1-Positive | |
| | | Cancer Cells. | |
| Teneobio patent anti- | 2022 WO2022183101 | | TeneoBio |
| MUC1-C | HARRIS, Katherine . . . | | |
| | ANTI-MUC1-C | | |
| | ANTIBODIES AND CAR-T | | |
| | STRUCTURES | | |
| Tokyo Med. U. patent | 2018 WO2019077951 | | Tokyo Med. U. |
| anti-MUC1 | KURODA Masahiko, . . . | | |
| | ANTIBODY AGAINST | | |
| | MUC1 OR ANTIGEN- | | |
| | BINDING FRAGMENT | | |
| | THEREOF, GENE | | |
| | ENCODING SAME, AND | | |
| | USE THEREOF | | |
| U. Penn. anti-Tn-MUC1 | 2020 WO2020198413 | 2020 Macías-León J, | U. Penn. |
| CAR | POSEY, Avery D., . . . TN- | Be . . . Structural | |
| | MUC1 CHIMERIC | characterization of an | |
| | ANTIGEN RECEPTOR | unprecedented lectin- | |
| | (CAR) T CELL THERAPY | like antitumoral anti- | |
| | View on Patents Page | MUC1 antibody. | |
| | | 2016 Posey AD, | |
| | | Schwab . . . Engineered | |
| | | CAR T Cells Targeting | |
| | | the Cancer-Associated | |
| | | Tn-Glycoform of the | |
| | | Membrane Mucin | |
| | | MUC1 Control | |
| | | Adenocarcinoma. | |
| Wenzhou Med. U. anti- | | 2022 Wu G, Li L, Liu | Wenzhou Med. U. |
| MUC1 | | M . . . Therapeutic effect | |
| | | of a MUC1-specific | |
| | | monoclonal antibody- | |
| | | drug conjugates | |
| | | against pancreatic | |
| | | cancer model. | |
| | | 2021 Wu G, Li L, Qiu | |
| | | Y . . . A novel humanized | |
| | | MUC1 antibody-drug | |
| | | conjugate for the | |
| | | treatment of | |
| | | trastuzumab-resistant | |
| | | breast cancer. | |
| Benhealth Biopharma | | 2018 NCT03509298 | Benhealth Biopharma |
| anti-CD3/MUC1 | | Phase 2 Study of | |
| | | Activated Cytokine- | |
| | | induced Killer Armed | |
| | | With Bispecific | |
| | | Antibody for Advanced | |
| | | Pancreatic Cancer | |
| | | 2018 NCT03524274 | |
| | | Phase 2 Study of | |
| | | Activated Cytokine- | |
| | | induced Killer Armed | |
| | | With Bispecific | |
| | | Antibody for Advanced | |
| | | Colorectal Cancer | |
| | | 2018 NCT03524261 | |
| | | Phase 2 Study of | |
| | | Activated Cytokine- | |
| | | induced Killer Armed | |
| | | With Bispecific | |
| | | Antibody for Advanced | |
| | | Breast Cancer | |
| | | 2018 NCT03540199 | |
| | | Phase 2 Study of | |
| | | Activated Cytokine- | |
| | | induced Killer Armed | |
| | | With Bispecific | |
| | | Antibody for Advanced | |
| | | Kidney Cancer | |
| | | 2018 NCT03554395 | |
| | | Phase 2 Study of | |
| | | Activated Cytokine- | |
| | | induced Killer Armed | |
| | | With Bispecific | |
| | | Antibody for Advanced | |
| | | Gastric Cancer | |
| | | 2018 NCT03484962 | |
| | | Phase 2 Study of | |
| | | Activated CIK Armed | |
| | | With Bispecific | |
| | | Antibody for Advanced | |
| | | Liver Cancer | |
| | | 2018 NCT03501056 | |
| | | Phase 2 Study of | |
| | | Activated Cytokine- | |
| | | induced Killer Armed | |
| | | With Bispecific | |
| | | Antibody for Advanced | |
| | | Lung Cancer | |
| | | 2017 NCT03146637 | |
| | | Phase 2 Study of PD-1 | |
| | | Activated CIK Armed | |
| | | With Bispecific | |
| | | Antibody for Advanced | |
| | | Liver Cancer | |
| Glycotope patent anti- | | | Glycotope |
| CD3/MUC1 | | | |
| Glycotope patent anti- | | | Glycotope |
| MUC1/PD-L1 | | | |
| Immunogen patent | 2016 US20160355605 | | ImmunoGen |
| anti-CA6 | PAYNE, Gillian; C . . . CA6 | | |
| | Antigen-Specific | | |
| | Cytotoxic Conjugate and | | |
| | Methods of Using the | | |
| | Same | | |
| | 2016 U.S. Pat. No. 9,822,183 Payne, | | |
| | Gillian; C . . . CA6 antigen- | | |
| | specific cytotoxic | | |
| | conjugate and methods | | |
| | of using the same | | |
| | 2014 US20150297762 | | |
| | Kellogg, Anne Eli . . . | | |
| | MONOCLONAL | | |
| | ANTIBODY DS6, TUMOR- | | |
| | ASSOCIATED ANTIGEN | | |
| | CA6, AND METHODS OF | | |
| | USE THEREOF | | |
| | 2014 U.S. Pat. No. 9,539,348 | | |
| | Kellogg, Anne Eli . . . | | |
| | Monoclonal antibody | | |
| | DS6, tumor-associated | | |
| | antigen CA6, and | | |
| | methods of use thereof | | |
| | 2014 WO2014122529 | | |
| | KRUIP, Jochen, SA . . . | | |
| | IMMUNO IMAGING | | |
| | AGENT FOR USE WITH | | |
| | ANTIBODY-DRUG | | |
| | CONJUGATE THERAPY | | |
| | 2014 WO2014124026 | | |
| | KRUIP, Jochen, GA . . . | | |
| | IMMUNO IMAGING | | |
| | AGENT FOR USE WITH | | |
| | ANTIBODY-DRUG | | |
| | CONJUGATE THERAPY | | |
| | 2014 US20140255305 | | |
| | Kruip, Jochen; Ga . . . | | |
| | IMMUNO IMAGING | | |
| | AGENT FOR USE WITH | | |
| | ANTIBODY-DRUG | | |
| | CONJUGATE THERAPY | | |
| | 2011 US20120045776 | | |
| | Kellogg, Anne Eli . . . | | |
| | MONOCLONAL | | |
| | ANTIBODY DS6, TUMOR- | | |
| | ASSOCIATED ANTIGEN | | |
| | CA6, AND METHODS OF | | |
| | USE THEREOF | | |
| | 2011 U.S. Pat. No. 8,728,741 | | |
| | Kellogg, Anne Eli . . . | | |
| | Monoclonal antibody | | |
| | DS6, tumor-associated | | |
| | antigen CA6, and | | |
| | methods of use thereof | | |
| | 2010 US20110064733 | | |
| | PAYNE, Gillian; C . . . CA6 | | |
| | ANTIGEN-SPECIFIC | | |
| | CYTOTOXIC CONJUGATE | | |
| | AND METHODS OF | | |
| | USING THE SAME | | |
| | 2010 U.S. Pat. No. 9,370,584 Payne, | | |
| | Gillian; C . . . CA6 antigen- | | |
| | specific cytotoxic | | |
| | conjugate and methods | | |
| | of using the same | | |
| | 2009 U.S. Pat. No. 8,067,186 | | |
| | Kellogg, Anne Eli . . . | | |
| | Monoclonal antibody | | |
| | DS6, tumor-associated | | |
| | antigen CA6, and | | |
| | methods of use thereof | | |
| | 2008 US20090099336 | | |
| | PAYNE, Gillian; C . . . CA6 | | |
| | Antigen-Specific | | |
| | Cytotoxic Conjugate and | | |
| | Methods of Using the | | |
| | Same | | |
| | 2008 U.S. Pat. No. 8,987,424 Payne, | | |
| | Gillian; C . . . CA6 antigen- | | |
| | specific cytotoxic | | |
| | conjugate and methods | | |
| | of using the same | | |
| | 2005 U.S. Pat. No. 7,834,155 Payne, | | |
| | Gillian; C . . . CA6 antigen- | | |
| | specific cytotoxic | | |
| | conjugate and methods | | |
| | of using the same | | |
| | 2001 WO2002016401 | | |
| | WENNERBERG, Anne, . . . | | |
| | MONOCLONAL | | |
| | ANTIBODY DS6, TUMOR- | | |
| | ASSOCIATED ANTIGEN | | |
| | CA6, AND METHODS OF | | |
| | USE THEREOF | | |
| M1231 | 2021 WO2022128716 | 2021 NCT04695847 | Merck Serono Sutro |
| | WINZER, Matthias, . . . | Phase 1 M1231 in | |
| | PHARMACEUTICAL | Participants With Solid | |
| | COMPOSITION | Tumors | |
| | COMPRISING A | | |
| | BISPECIFIC ANTI- | | |
| | MUC1/EGFR ANTIBODY- | | |
| | DRUG CONJUGATE | | |
| | 2021 WO2021247798 | | |
| | YAM, Alice, KNUEH . . . | | |
| | BISPECIFIC ANTIBODY- | | |
| | DRUG CONJUGATES | | |
| | TARGETING EGFR AND | | |
| | MUC1 AND USES | | |
| | THEREOF | | |
| Moffitt Cancer Center | 2022 WO2022272283 | | Moffitt Cancer Center |
| patent anti-EGFR/ | DAVILA, Marco L. DUAL | | |
| MUC1 CAR | EGFR-MUC1 CHIMERIC | | |
| | ANTIGEN RECEPTOR T | | |
| | CELLS | | |
| | 2021 WO2021247525 | | |
| | DAVILA, Marco L. DUAL | | |
| | EGFR-MUC1 CHIMERIC | | |
| | ANTIGEN RECEPTOR T | | |
| | CELLS | | |
| SAR566658 | 2016 U.S. Pat. No. 9,822,183 Payne, | 2010 NCT01156870 | ImmunoGen Sanofi |
| | Gillian; C . . . CA6 antigen- | Phase 1 First in Man | |
| | specific cytotoxic | Study of SAR566658 | |
| | conjugate and methods | Administered Every 3 | |
| | of using the same | Weeks in Patients With | |
| | 2014 WO2015014879 | DS6-positive and | |
| | ASSADOURIAN, Sylv . . . | Refractory Solid | |
| | USE OF ANTI-MUC1 | Tumors | |
| | MAYTANSINOID | 2017 Bouillon- | |
| | IMMUNOCONJUGATE | Pichault . . . Translational | |
| | ANTIBODY FOR THE | Model-Based Strategy | |
| | TREATMENT OF SOLID | to Guide the Choice of | |
| | TUMORS | Clinical Doses for | |
| | 2014 US20160347856 | Antibody-Drug | |
| | Assadourian, Sylv . . . Use | Conjugates. | |
| | of Anti-MUC1 | 2015 Ilovich O, | |
| | Maytansinoid | Natara . . . Development | |
| | Immunoconjugate | and Validation of an | |
| | Antibody for the | Immuno-PET Tracer as | |
| | Treatment of Solid | a Companion | |
| | Tumors | Diagnostic Agent for | |
| | 2014 US20140256916 | Antibody-Drug | |
| | Kruip, Jochen; Sa . . . | Conjugate Therapy to | |
| | IMMUNO IMAGING | Target the CA6 | |
| | AGENT FOR USE WITH | Epitope. | |
| | ANTIBODY-DRUG | ASCO 2016 A phase I | |
| | CONJUGATE THERAPY | study of SAR566658, | |
| | 2014 U.S. Pat. No. 9,844,607 Kruip, | an anti CA6-antibody | |
| | Jochen; Ga . . . Immuno | drug conjugate (ADC), | |
| | imaging agent for use | in patients (Pts) with | |
| | with antibody-drug | CA6-positive advanced | |
| | conjugate therapy | solid tumors | |
| | 2014 U.S. Pat. No. 9,989,524 Kruip, | (STs)(NCT01156870) | |
| | Jochen; Sa . . . Immuno | Carlos Alberto Go . . . | |
| | imaging agent for use | | |
| | with antibody-drug | | |
| | conjugate therapy | | |
| | 2004 WO2005009369 | | |
| | PAYNE, Gillian; C . . . A CA6 | | |
| | ANTIGEN-SPECIFIC | | |
| | CYTOTOXIC CONJUGATE | | |
| | AND METHODS OF | | |
| | USING THE SAME | | |
| | 2003 WO2004005470 | | |
| | PAYNE, Gillian; C . . . | | |
| | ANTIBODIES TO NON- | | |
| | SHED MUC1 AND | | |
| | MUC16, AND USES | | |
| | THEREOF | | |
| | US20180127511 PAYNE, | | |
| | Gillian; C . . . CA6 Antigen- | | |
| | Specific Cytotoxic | | |
| | Conjugate and Methods | | |
| | of Using the Same | | |
| Benhealth Biopharma | 2017 WO2017219974 | | Benhealth Biopharma |
| anti-CD16/MUC1 | YU, Haoyang, [symbol] , . . . | | |
| | BISPECIFIC ANTIBODY | | |
| | AND ANTIBODY | | |
| | CONJUGATE FOR | | |
| | TUMOUR THERAPY AND | | |
| | USE THEREOF | | |
| | 2017 US20190276554 | | |
| | YU, Haoyang; WANG . . . | | |
| | BISPECIFIC ANTIBODY | | |
| | AND ANTIBODY | | |
| | CONJUGATE FOR | | |
| | TUMOUR THERAPY AND | | |
| | USE THEREOF | | |
| | 2017 U.S. Pat. No. 11,525,009 Yu, | | |
| | Haoyang; Wang . . . | | |
| | Bispecific antibody and | | |
| | antibody conjugate for | | |
| | tumor therapy and use | | |
| | thereof | | |
| | 2016 US20180021440 | | |
| | YU, Haoyang; LI, . . . | | |
| | BISPECIFIC ANTIBODY | | |
| | CAPABLE OF BEING | | |
| | COMBINED WITH | | |
| | IMMUNE CELLS TO | | |
| | ENHANCE TUMOR | | |
| | KILLING CAPABILITY, | | |
| | AND PREPARATION | | |
| | METHOD THEREFOR | | |
| | AND APPLICATION | | |
| | THEREOF | | |
| | 2016 U.S. Pat. No. 10,758,625 Yu, | | |
| | Haoyang (Shen . . . | | |
| | Bispecific antibody | | |
| | capable of being | | |
| | combined with immune | | |
| | cells to enhance tumor | | |
| | killing capability, and | | |
| | preparation method | | |
| | therefor and application | | |
| | thereof | | |
| | 2016 WO2016165632 | | |
| | YU, Haoyang, LI, . . . | | |
| | BISPECIFIC ANTIBODY | | |
| | CAPABLE OF BEING | | |
| | COMBINED WITH | | |
| | IMMUNE CELLS TO | | |
| | ENHANCE TUMOR | | |
| | KILLING CAPABILITY, | | |
| | AND PREPARATION | | |
| | METHOD THEREFOR | | |
| | AND APPLICATION | | |
| | THEREOF | | |

### F. Chimeric Autoantibody Receptors

Provided herein are hypoimmunogenic cells comprising a chimeric autoantibody receptor (CAAR). A CAAR recognizes and binds to the target autoantibodies, e.g., expressed on autoreactive cells (e.g., autoreactive B-cells).

In some embodiments, a CAAR comprises an antigen, e.g., an autoantigen that can be bound by autoantibodies. In some embodiments, a CAAR comprises a transmembrane domain. In some embodiments, a CAAR comprises a signaling domain. In some embodiments, a CAAR comprises one or more signaling domains. In some embodiments, a CAAR comprises an antigen, a transmembrane domain, and a signaling domain. In some embodiments, a CAAR comprises an antigen, a transmembrane domain, and one or more signaling domains.

A CAAR can be expressed by, e.g., a hypoimmunogenic T-cell. Thus, the present disclosure provides CAAR-T cells. CAAR T-cells can recognize and can bind target autoantibodies expressed on autoreactive cells via an antigen of a CAAR. Once a CAAR T-cell binds a target autoantibody expressed on an autoreactive cell, the CAAR T-cell can destroy the autoreactive cell.

A CAAR can be expressed by, e.g., a hypoimmunogenic NK-cell. Thus, the present disclosure provides CAAR NK-cells. CAAR NK-cells can recognize and can bind target autoantibodies expressed on autoreactive cells via an antigen of a CAAR. Once a CAAR NK-cell binds a target autoantibody expressed on an autoreactive cell, the CAAR NK-cell can destroy the autoreactive cell.

### 1. CAAR Antigens

As discussed above, provided herein are CAARs comprising an antigen. Antigens in CAARs as provided are generally known to be bound by autoantibodies. In some embodiments, autoantibodies bind autoantigens associated with an autoimmune disease. Autoantigens associated with various autoimmune diseases can be determined. For example, certain autoantibodies and the associated autoimmune disease are provided in Table 31 below:

TABLE 31
Exemplary Autoantibodies

| Disease | Autoantigen(s) |
|---|---|
| Diabetes | Pancreatic β-cell Antigen |
| Rheumatoid Arthritis | Synovial Joint Antigen |
| Expermental Autoimmune | Myelin Basic Protein, Proteolipid Protein, |
| Encephalomyelities | Myelin Oligodendritic Glycoprotein |
| Multiple Sclerosis | Myelin Basic Protein, Proteolipid Protein, |
| | Myelin Oligodendritic Glycoprotein |
| Myastenia gravis | MuSK |
| Pemphigus Vulgaris | Keratinocyte Adhesion Protien Desmoglein 3 |
| | (Dsg3) |
| Sjogren's Syndrome | Ro-RNP Complex, La antigen |
| Vasculitis | Myeloperoxidase, proteinase 3, Cardiolipin |
| Wegener's Granulomatosis | Myeloperoxidase, proteinase 3, Cardiolipin |
| Rheumatoid Arthritis | Citrullinated Proteins, Carbamylated Proteins |
| Goodpasture's Syndrome | α3 Chain of basement membrane collagen |

### 2. Transmembrane Domain

In some embodiments, a CAAR transmembrane domain comprises at least a transmembrane region of the alpha, beta or zeta chain of a T cell receptor, CD28, CD3 epsilon, CD45, CD4, CD5, CD8, CD9, CD16, CD22, CD33, CD37, CD64, CD80, CD86, CD134, CD137, CD154, or functional variant thereof. In some embodiments, a transmembrane domain comprises at least a transmembrane region(s) of CD8α, CD8β, 4-1BB/CD137, CD28, CD34, CD4, FcERIγ, CD16, OX40/CD134, CD3, CD3ε, CD3γ, CD3δ, TCRα, TCRβ, TCRζ, CD32, CD64, CD64, CD45, CD5, CD9, CD22, CD37, CD80, CD86, CD40, CD40L/CD154, VEGFR2, FAS, and FGFR2B, or functional variant thereof.

### 3. Signaling Domain or Plurality of Signaling Domains

In some embodiments, a CAAR described herein comprises one or at least one signaling domain selected from one or more of B7-1/CD80; B7-2/CD86; B7-H1/PD-L1; B7-H2; B7-H3; B7-H4; B7-H6; B7-H7; BTLA/CD272; CD28; CTLA-4; Gi24/VISTA/B7-H5; ICOS/CD278; PD-1; PD-L2/B7-DC; PDCD6); 4-1BB/TNFSF9/CD137; 4-1BB Ligand/TNFSF9; BAFF/BLyS/TNFSF13B; BAFF R/TNFRSF13C; CD27/TNFRSF7; CD27 Ligand/TNFSF7; CD30/TNFRSF8; CD30 Ligand/TNFSF8; CD40/TNFRSF5; CD40/TNFSF5; CD40 Ligand/TNFSF5; DR3/TNFRSF25; GITR/TNFRSF18; GITR Ligand/TNFSF18; HVEM/TNFRSF14; LIGHT/TNFSF14; Lymphotoxin-alpha/TNF-beta; OX40/TNFRSF4; OX40 Ligand/TNFSF4; RELT/TNFRSF19L; TACI/TNFRSF13B; TL1A/TNFSF15; TNF-alpha; TNF RII/TNFRSF1B); 2B4/CD244/SLAMF4; BLAME/SLAMF8; CD2; CD2F-10/SLAMF9; CD48/SLAMF2; CD58/LFA-3; CD84/SLAMF5; CD229/SLAMF3; CRACC/SLAMF7; NTB-A/SLAMF6; SLAM/CD150); CD2; CD7; CD53; CD82/Kai-1; CD90/Thy1; CD96; CD160; CD200; CD300a/LMIR1; HLA Class I; HLA-DR; Ikaros; Integrin alpha 4/CD49d; Integrin alpha 4 beta 1; Integrin alpha 4 beta 7/LPAM-1; LAG-3; TCL1A; TCL1B; CRTAM; DAP12; Dectin-1/CLEC7A; DPPIV/CD26; EphB6; TIM-1/KIM-1/HAVCR; TIM-4; TSLP; TSLP R; lymphocyte function associated antigen-1 (LFA-1); NKG2C, a CD3 zeta domain, an immunoreceptor tyrosine-based activation motif (ITAM), CD27, CD28, 4-1BB, CD134/OX40, CD30, CD40, PD-1, ICOS, lymphocyte function-associated antigen-1 (LFA-1), CD2, CD7, LIGHT, NKG2C, B7-H3, a ligand that specifically binds with CD83, or functional fragment thereof.

In some embodiments, the at least one signaling domain comprises a CD3 zeta domain or an immunoreceptor tyrosine-based activation motif (ITAM), or functional variant thereof. In other embodiments, the at least one signaling domain comprises (i) a CD3 zeta domain, or an immunoreceptor tyrosine-based activation motif (ITAM), or functional variant thereof; and (ii) a CD28 domain, or a 4-1BB domain, or functional variant thereof. In yet other embodiments, the at least one signaling domain comprises a (i) a CD3 zeta domain, or an immunoreceptor tyrosine-based activation motif (ITAM), or functional variant thereof; (ii) a CD28 domain or functional variant thereof; and (iii) a 4-1BB domain, or a CD134 domain, or functional variant thereof. In some embodiments, the at least one signaling domain comprises a (i) a CD3 zeta domain, or an immunoreceptor tyrosine-based activation motif (ITAM), or functional variant thereof; (ii) a CD28 domain or functional variant thereof; (iii) a 4-1BB domain, or a CD134 domain, or functional variant thereof; and (iv) a cytokine or costimulatory ligand transgene.

In some embodiments, the at least two signaling domains comprise a CD3 zeta domain or an immunoreceptor tyrosine-based activation motif (ITAM), or functional variant thereof. In other embodiments, the at least two signaling domains comprise (i) a CD3 zeta domain, or an immunoreceptor tyrosine-based activation motif (ITAM), or functional variant thereof; and (ii) a CD28 domain, or a 4-1BB domain, or functional variant thereof. In yet other embodiments, the at least one signaling domain comprises a (i) a CD3 zeta domain, or an immunoreceptor tyrosine-based activation motif (ITAM), or functional variant thereof; (ii) a CD28 domain or functional variant thereof; and (iii) a 4-1BB domain, or a CD134 domain, or functional variant thereof. In some embodiments, the at least two signaling domains comprise a (i) a CD3 zeta domain, or an immunoreceptor tyrosine-based activation motif (ITAM), or functional variant thereof; (ii) a CD28 domain or functional variant thereof; (iii) a 4-1BB domain, or a CD134 domain, or functional variant thereof; and (iv) a cytokine or costimulatory ligand transgene.

In some embodiments, the at least three signaling domains comprise a CD3 zeta domain or an immunoreceptor tyrosine-based activation motif (ITAM), or functional variant thereof. In other embodiments, the at least three signaling domains comprise (i) a CD3 zeta domain, or an immunoreceptor tyrosine-based activation motif (ITAM), or functional variant thereof; and (ii) a CD28 domain, or a 4-1BB domain, or functional variant thereof. In yet other embodiments, the least three signaling domains comprises a (i) a CD3 zeta domain, or an immunoreceptor tyrosine-based activation motif (ITAM), or functional variant thereof; (ii) a CD28 domain or functional variant thereof; and (iii) a 4-1BB domain, or a CD134 domain, or functional variant thereof. In some embodiments, the at least three signaling domains comprise a (i) a CD3 zeta domain, or an immunoreceptor tyrosine-based activation motif (ITAM), or functional variant thereof; (ii) a CD28 domain or functional variant thereof; (iii) a 4-1BB domain, or a CD134 domain, or functional variant thereof; and (iv) a cytokine or costimulatory ligand transgene.

In some embodiments, the CAAR comprises a CD3 zeta domain or an immunoreceptor tyrosine-based activation motif (ITAM), or functional variant thereof. In some embodiments, the CAAR comprises (i) a CD3 zeta domain, or an immunoreceptor tyrosine-based activation motif (ITAM), or functional variant thereof; and (ii) a CD28 domain, or a 4-1BB domain, or functional variant thereof.

In some embodiments, the CAAR comprises a (i) a CD3 zeta domain, or an immunoreceptor tyrosine-based activation motif (ITAM), or functional variant thereof; (ii) a CD28 domain or functional variant thereof; and (iii) a 4-1BB domain, or a CD134 domain, or functional variant thereof.

In some embodiments, the CAAR comprises (i) a CD3 zeta domain, or an immunoreceptor tyrosine-based activation motif (ITAM), or functional variant thereof; (ii) a CD28 domain, or a 4-1BB domain, or functional variant thereof, and/or (iii) a 4-1BB domain, or a CD134 domain, or functional variant thereof.

In some embodiments, the CAAR comprises a (i) a CD3 zeta domain, or an immunoreceptor tyrosine-based activation motif (ITAM), or functional variant thereof; (ii) a CD28 domain or functional variant thereof; (iii) a 4-1BB domain, or a CD134 domain, or functional variant thereof; and (iv) a cytokine or costimulatory ligand transgene.

### G. Chimeric B-Cell-Targeting Antibody Receptors

Provided herein are hypoimmunogenic cells comprising a chimeric B-cell autoantibody receptor (BAR). A BAR recognizes and binds to certain antibody-expressing B cells.

In some embodiments, a BAR comprises an antigen. An antigen of a BAR can be bound by neutralizing antibodies. The neutralizing antibodies may be undesirable because they can block or inhibit an effect or function of antigen to which they bind. For example, hemophilia patients can receive therapeutic factor VIII (FVIII) as part of their treatment. However, a patient's body may develop an immune response against the FVIII, including the production of anti-FVIII antibodies from B cells. When the patient produces anti-FVIII antibodies that bind to FVIII, FVIII is not able to perform its therapeutic functions. Accordingly, it may be beneficial to remove the anti-FVIII antibodies and/or the B-cells producing those antibodies from the patient. A BAR, which includes an FVIII antigen, can be used for this purpose.

In some embodiments, a BAR comprises a transmembrane domain. In some embodiments, a BAR comprises a signaling domain. In some embodiments, a BAR comprises one or more signaling domains.

In some embodiments, a BAR comprises an antigen, a transmembrane domain, and a signaling domain. In some embodiments, a BAR comprises an antigen, a transmembrane domain, and one or more signaling domains.

A BAR can be expressed by, e.g., a hypoimmunogenic T-cell. Thus, the present disclosure provides BAR T-cells. BAR T-cells can recognize and can bind target select antibodies and/or the B cells producing those antibodies. Once a BAR T-cell binds a target antibody, the BAR T-cell can destroy the antibodies and/or the B cells producing those antibodies. In some embodiments, a BAR T-cell is a BAR T-cell (Treg), e.g., a regulatory T-cell (Treg) comprising a BAR.

A BAR can be expressed by, e.g., a hypoimmunogenic NK-cell. Thus, the present disclosure provides BAR NK-cells. BAR NK-cells can recognize and can bind target select antibodies and/or the B cells producing those antibodies. Once a BAR NK-cell binds a target antibody, the BAR NK-cell can destroy the antibodies and/or the B cells producing those antibodies.

### 1. BAR Antigens

As discussed above, provided herein are BARs comprising an antigen. Antigens in BARs as provided are generally known to be bound by autoantibodies. An antigen of a BAR can be bound by neutralizing antibodies. The neutralizing antibodies may be undesirable because they can block or inhibit an effect or function of antigen to which they bind.

### 2. Transmembrane Domain

In some embodiments, a BAR transmembrane domain comprises at least a transmembrane region of the alpha, beta or zeta chain of a T cell receptor, CD28, CD3 epsilon, CD45, CD4, CD5, CD8, CD9, CD16, CD22, CD33, CD37, CD64, CD80, CD86, CD134, CD137, CD154, or functional variant thereof. In some embodiments, a transmembrane domain comprises at least a transmembrane region(s) of CD8α, CD8β, 4-1BB/CD137, CD28, CD34, CD4, FcERIγ, CD16, OX40/CD134, CD3ζ, CD3ε, CD3γ, CD3δ, TCRα, TCRβ, TCRζ, CD32, CD64, CD64, CD45, CD5, CD9, CD22, CD37, CD80, CD86, CD40, CD40L/CD154, VEGFR2, FAS, and FGFR2B, or functional variant thereof.

### 3. Signaling Domain or Plurality of Signaling Domains

In some embodiments, a BAR described herein comprises one or at least one signaling domain selected from one or more of B7-1/CD80; B7-2/CD86; B7-H1/PD-L1; B7-H2; B7-H3; B7-H4; B7-H6; B7-H7; BTLA/CD272; CD28; CTLA-4; Gi24/VISTA/B7-H5; ICOS/CD278; PD-1; PD-L2/B7-DC; PDCD6); 4-1BB/TNFSF9/CD137; 4-1BB Ligand/TNFSF9; BAFF/BLyS/TNFSF13B; BAFF R/TNFRSF13C; CD27/TNFRSF7; CD27 Ligand/TNFSF7; CD30/TNFRSF8; CD30 Ligand/TNFSF8; CD40/TNFRSF5; CD40/TNFSF5; CD40 Ligand/TNFSF5; DR3/TNFRSF25; GITR/TNFRSF18; GITR Ligand/TNFSF18; HVEM/TNFRSF14; LIGHT/TNFSF14; Lymphotoxin-alpha/TNF-beta; OX40/TNFRSF4; OX40 Ligand/TNFSF4; RELT/TNFRSFi9L; TACI/TNFRSFi3B; TL1A/TNFSF15; TNF-alpha; TNF RII/TNFRSF1B); 2B4/CD244/SLAMF4; BLAME/SLAMF8; CD2; CD2F-10/SLAMF9; CD48/SLAMF2; CD58/LFA-3; CD84/SLAMF5; CD229/SLAMF3; CRACC/SLAMF7; NTB-A/SLAMF6; SLAM/CD150); CD2; CD7; CD53; CD82/Kai-1; CD90/Thy1; CD96; CD160; CD200; CD300a/LMIR1; HLA Class I; HLA-DR; Ikaros; Integrin alpha 4/CD49d; Integrin alpha 4 beta 1; Integrin alpha 4 beta 7/LPAM-1; LAG-3; TCL1A; TCL1B; CRTAM; DAP12; Dectin-1/CLEC7A; DPPIV/CD26; EphB6; TIM-1/KIM-1/HAVCR; TIM-4; TSLP; TSLP R; lymphocyte function associated antigen-1 (LFA-1); NKG2C, a CD3 zeta domain, an immunoreceptor tyrosine-based activation motif (ITAM), CD27, CD28, 4-1BB, CD134/OX40, CD30, CD40, PD-1, ICOS, lymphocyte function-associated antigen-1 (LFA-1), CD2, CD7, LIGHT, NKG2C, B7-H3, a ligand that specifically binds with CD83, or functional fragment thereof.

In some embodiments, the at least one signaling domain comprises a CD3 zeta domain or an immunoreceptor tyrosine-based activation motif (ITAM), or functional variant thereof. In other embodiments, the at least one signaling domain comprises (i) a CD3 zeta domain, or an immunoreceptor tyrosine-based activation motif (ITAM), or functional variant thereof; and (ii) a CD28 domain, or a 4-1BB domain, or functional variant thereof. In yet other embodiments, the at least one signaling domain comprises a (i) a CD3 zeta domain, or an immunoreceptor tyrosine-based activation motif (ITAM), or functional variant thereof; (ii) a CD28 domain or functional variant thereof; and (iii) a 4-1BB domain, or a CD134 domain, or functional variant thereof. In some embodiments, the at least one signaling domain comprises a (i) a CD3 zeta domain, or an immunoreceptor tyrosine-based activation motif (ITAM), or functional variant thereof; (ii) a CD28 domain or functional variant thereof; (iii) a 4-1BB domain, or a CD134 domain, or functional variant thereof; and (iv) a cytokine or costimulatory ligand transgene.

In some embodiments, the at least two signaling domains comprise a CD3 zeta domain or an immunoreceptor tyrosine-based activation motif (ITAM), or functional variant thereof. In other embodiments, the at least two signaling domains comprise (i) a CD3 zeta domain, or an immunoreceptor tyrosine-based activation motif (ITAM), or functional variant thereof; and (ii) a CD28 domain, or a 4-1BB domain, or functional variant thereof. In yet other embodiments, the at least one signaling domain comprises a (i) a CD3 zeta domain, or an immunoreceptor tyrosine-based activation motif (ITAM), or functional variant thereof; (ii) a CD28 domain or functional variant thereof; and (iii) a 4-1BB domain, or a CD134 domain, or functional variant thereof. In some embodiments, the at least two signaling domains comprise a (i) a CD3 zeta domain, or an immunoreceptor tyrosine-based activation motif (ITAM), or functional variant thereof; (ii) a CD28 domain or functional variant thereof; (iii) a 4-1BB domain, or a CD134 domain, or functional variant thereof; and (iv) a cytokine or costimulatory ligand transgene.

In some embodiments, the at least three signaling domains comprise a CD3 zeta domain or an immunoreceptor tyrosine-based activation motif (ITAM), or functional variant thereof. In other embodiments, the at least three signaling domains comprise (i) a CD3 zeta domain, or an immunoreceptor tyrosine-based activation motif (ITAM), or functional variant thereof; and (ii) a CD28 domain, or a 4-1BB domain, or functional variant thereof. In yet other embodiments, the least three signaling domains comprises a (i) a CD3 zeta domain, or an immunoreceptor tyrosine-based activation motif (ITAM), or functional variant thereof; (ii) a CD28 domain or functional variant thereof; and (iii) a 4-1BB domain, or a CD134 domain, or functional variant thereof. In some embodiments, the at least three signaling domains comprise a (i) a CD3 zeta domain, or an immunoreceptor tyrosine-based activation motif (ITAM), or functional variant thereof; (ii) a CD28 domain or functional variant thereof; (iii) a 4-1BB domain, or a CD134 domain, or functional variant thereof; and (iv) a cytokine or costimulatory ligand transgene.

In some embodiments, the BAR comprises a CD3 zeta domain or an immunoreceptor tyrosine-based activation motif (ITAM), or functional variant thereof. In some embodiments, the BAR comprises (i) a CD3 zeta domain, or an immunoreceptor tyrosine-based activation motif (ITAM), or functional variant thereof; and (ii) a CD28 domain, or a 4-1BB domain, or functional variant thereof.

In some embodiments, the BAR comprises a (i) a CD3 zeta domain, or an immunoreceptor tyrosine-based activation motif (ITAM), or functional variant thereof; (ii) a CD28 domain or functional variant thereof; and (iii) a 4-1BB domain, or a CD134 domain, or functional variant thereof.

In some embodiments, the BAR comprises (i) a CD3 zeta domain, or an immunoreceptor tyrosine-based activation motif (ITAM), or functional variant thereof; (ii) a CD28 domain, or a 4-1BB domain, or functional variant thereof, and/or (iii) a 4-1BB domain, or a CD134 domain, or functional variant thereof.

In some embodiments, the BAR comprises a (i) a CD3 zeta domain, or an immunoreceptor tyrosine-based activation motif (ITAM), or functional variant thereof; (ii) a CD28 domain or functional variant thereof; (iii) a 4-1BB domain, or a CD134 domain, or functional variant thereof; and (iv) a cytokine or costimulatory ligand transgene.

H. Therapeutic Cells from Primary T Cells

Provided herein are hypoimmunogenic cells including, but not limited to, primary T cells that evade immune recognition. In some embodiments, the engineered CAR-T cells are produced (e.g., generated, cultured, or derived) from T cells such as primary T cells. In some instances, primary T cells are obtained (e.g., harvested, extracted, removed, or taken) from a subject or an individual. In some embodiments, primary T cells are produced from a pool of T cells such that the T cells are from one or more subjects (e.g., one or more human including one or more healthy humans). In some embodiments, the pool of primary T cells is from 1-100, 1-50, 1-20, 1-10, 1 or more, 2 or more, 3 or more, 4 or more, 5 or more, 10 or more, 20 or more, 30 or more, 40 or more, 50 or more, or 100 or more subjects. In some embodiments, the donor subject is different from the patient (e.g., the recipient that is administered the therapeutic cells). In some embodiments, the pool of T cells do not include cells from the patient. In some embodiments, one or more of the donor subjects from which the pool of T cells is obtained are different from the patient.

In some embodiments, the engineered CAR-T cells do not activate an innate and/or an adaptive immune response in the patient (e.g., recipient upon administration). Provided are methods of treating a disorder by administering a population of hypoimmunogenic cells to a subject (e.g., recipient) or patient in need thereof. In some embodiments, the engineered CAR-T cells described herein comprise T cells engineered (e.g., are modified) to express a chimeric antigen receptor including but not limited to a chimeric antigen receptor described herein. In some instances, the T cells are populations or subpopulations of primary T cells from one or more individuals. In some embodiments, the T cells described herein such as the engineered or modified T cells comprise reduced expression of an endogenous T cell receptor.

In some embodiments, the present disclosure is directed to hypoimmunogenic primaryT cells that overexpress CD47 and CARs, and have reduced expression or lack expression of MHC class I and/or MHC class II human leukocyte antigens and have reduced expression or lack expression of TCR complex molecules. The cells outlined herein overexpress CD47 and CARs and evade immune recognition. In some embodiments, the primary T cells display reduced levels or activity of MHC class I antigens, MHC class II antigens, and/or TCR complex molecules. In certain embodiments, primary T cells overexpress CD47 and CARs and harbor a genomic modification in the B2M gene. In some embodiments, T cells overexpress CD47 and CARs and harbor a genomic modification in the CIITA gene. In some embodiments, primary T cells overexpress CD47 and CARs and harbor a genomic modification in the TRAC gene. In some embodiments, primary T cells overexpress CD47 and CARs and harbor a genomic modification in the TRB gene. In some embodiments, T cells overexpress CD47 and CARs and harbor genomic modifications in one or more of the following genes: the B2M, CIITA, TRAC and TRB genes.

Exemplary T cells of the present disclosure are selected from the group consisting of cytotoxic T cells, helper T cells, memory T cells, central memory T cells, effector memory T cells, effector memory RA T cells, regulatory T cells, tissue infiltrating lymphocytes, and combinations thereof. In certain embodiments, the T cells express CCR7, CD27, CD28, and CD45RA. In some embodiments, the central T cells express CCR7, CD27, CD28, and CD45RO. In other embodiments, the effector memory T cells express PD-1, CD27, CD28, and CD45RO. In other embodiments, the effector memory RA T cells express PD-1, CD57, and CD45RA.

In some embodiments, the T cell is a modified (e.g., an engineered) T cell. In some cases, the modified T cell comprise a modification causing the cell to express at least one chimeric antigen receptor that specifically binds to an antigen or epitope of interest expressed on the surface of at least one of a damaged cell, a dysplastic cell, an infected cell, an immunogenic cell, an inflamed cell, a malignant cell, a metaplastic cell, a mutant cell, and combinations thereof. In other cases, the modified T cell comprise a modification causing the cell to express at least one protein that modulates a biological effect of interest in an adjacent cell, tissue, or organ when the cell is in proximity to the adjacent cell, tissue, or organ. Useful modifications to primary T cells are described in detail in US2016/0348073 and WO2020/018620, the disclosures of which are incorporated herein in their entireties.

In some embodiments, the engineered CAR-T cells described herein comprise T cells that are engineered (e.g., are modified) to express a chimeric antigen receptor including but not limited to a chimeric antigen receptor described herein. In some instances, the T cells are populations or subpopulations of primary T cells from one or more individuals. In some embodiments, the T cells described herein such as the engineered or modified T cells include reduced expression of an endogenous T cell receptor. In some embodiments, the T cells described herein such as the engineered or modified T cells include reduced expression of cytotoxic T-lymphocyte-associated protein 4 (CTLA-4). In other embodiments, the T cells described herein such as the engineered or modified T cells include reduced expression of programmed cell death (PD-1). In certain embodiments, the T cells described herein such as the engineered or modified T cells include reduced expression of CTLA-4 and PD-1. Methods of reducing or eliminating expression of CTLA-4, PD-1 and both CTLA-4 and PD-1 can include any recognized by those skilled in the art, such as but not limited to, genetic modification technologies that utilize rare-cutting endonucleases and RNA silencing or RNA interference technologies. Non-limiting examples of a rare-cutting endonuclease include any Cas protein, TALEN, zinc finger nuclease, meganuclease, and homing endonuclease. In some embodiments, an exogenous nucleic acid encoding a polypeptide as disclosed herein (e.g., a chimeric antigen receptor, CD47, or another tolerogenic factor disclosed herein) is inserted at a CTLA-4 and/or PD-1 gene locus. In some embodiments, the exogenous polynucleotide is inserted into at least one allele of the cell using viral transduction, for example, with a vector. In some embodiments, the vector is a pseudotyped, self-inactivating lentiviral vector that carries the exogenous polynucleotide. In some embodiments, the vector is a self-inactivating lentiviral vector pseudotyped with a vesicular stomatitis VSV-G envelope, and which carries the exogenous polynucleotide. In some embodiments, the exogenous polynucleotide is inserted into at least one allele of the cell using viral transduction. In some embodiments, the exogenous polynucleotide is inserted into at least one allele of the cell using a lentivirus based viral vector.

In some embodiments, the T cells described herein such as the engineered or modified T cells include enhanced expression of PD-1.

In some embodiments, the hypoimmunogenic T cell includes a polynucleotide encoding a CAR, wherein the polynucleotide is inserted in a genomic locus. In some embodiments, the polynucleotide encoding the CAR is randomly integrated into the genome of the cell. In some embodiments, the polynucleotide encoding the CAR is randomly integrated into the genome of the cell via viral vector transduction. In some embodiments, the polynucleotide encoding the CAR is randomly integrated into the genome of the cell via lentiviral vector transduction. In some embodiments, the polynucleotide is inserted into a safe harbor or target locus, such as but not limited to, an AAVS1, CCR5, CLYBL, ROSA26, SHS231, F3 (also known as CD142), MICA, MICB, LRP1 (also known as CD91), HMGB1, ABO, RHD, FUT1, or KDM5D gene locus. In some embodiments, the polynucleotide is inserted in a B2M, CIITA, TRAC, TRB, PD-1 or CTLA-4 gene.

In some embodiments, the hypoimmunogenic T cell includes a polynucleotide encoding a CAR that is expressed in a cell using an expression vector. In some embodiments, the CAR is introduced to the cell using a viral expression vector that mediates integration of the CAR sequence into the genome of the cell. For example, the expression vector for expressing the CAR in a cell comprises a polynucleotide sequence encoding the CAR. The expression vector can be an inducible expression vector. The expression vector can be a viral vector, such as but not limited to, a lentiviral vector.

Hypoimmunogenic T cells provided herein are useful for the treatment of suitable cancers including, but not limited to, lymphoma, leukemia, B cell acute lymphoblastic leukemia (B-ALL), diffuse large B-cell lymphoma, B-cell Non-Hodgkin lymphoma (B-NHL), B-cell chronic lymphoblastic leukemia, liver cancer, pancreatic cancer, breast cancer, ovarian cancer, colorectal cancer, lung cancer, non-small cell lung cancer, acute myeloid lymphoid leukemia, multiple myeloma, gastric cancer, gastric adenocarcinoma, pancreatic adenocarcinoma, glioblastoma, neuroblastoma, lung squamous cell carcinoma, hepatocellular carcinoma, and bladder cancer. In some embodiments, any of the exemplary cancers are also a CD19-negative cancer, a CD22-positive cancer, a CD19-negative/CD22-positive cancer, or a CD19-positive cancer. In certain embodiments, any of the exemplary cancers underwent antigen evasion and no longer express an antigen or have reduced expression of an antigen previously expressed. For example, any of the exemplary cancers can be a CD19-negative and a CD22-positive cancer but were previously CD19-positive and CD22-negative or CD22-positive.

I. Therapeutic Cells Differentiated from Hypoimmunogenic Pluripotent Stem Cells

Provided herein are hypoimmunogenic cells including, cells derived from pluripotent stem cells, that evade immune recognition. In some embodiments, the cells do not activate an innate and/or an adaptive immune response in the patient or subject (e.g., recipient upon administration). Provided are methods of treating a disorder comprising repeat dosing of a population of hypoimmunogenic cells to a recipient subject in need thereof.

In some embodiments, the pluripotent stem cell and any cell differentiated from such a pluripotent stem cell is modified to exhibit reduced expression of MHC class I human leukocyte antigens. In other embodiments, the pluripotent stem cell and any cell differentiated from such a pluripotent stem cell is modified to exhibit reduced expression of MHC class II human leukocyte antigens. In certain embodiments, the pluripotent stem cell and any cell differentiated from such a pluripotent stem cell is modified to exhibit reduced expression of TCR complexes. In some embodiments, the pluripotent stem cell and any cell differentiated from such a pluripotent stem cell is modified to exhibit reduced expression of MHC class I and II human leukocyte antigens. In some embodiments, the pluripotent stem cell and any cell differentiated from such a pluripotent stem cell is modified to exhibit reduced expression of MHC class I and II human leukocyte antigens and TCR complexes.

In some embodiments, the pluripotent stem cell and any cell differentiated from such a pluripotent stem cell is modified to exhibit reduced expression of MHC class I and/or II human leukocyte antigens and exhibit increased CD47 expression. In some instances, the cell overexpresses CD47 by harboring one or more CD47 transgenes. In some embodiments, the pluripotent stem cell and any cell differentiated from such a pluripotent stem cell is modified to exhibit reduced expression of MHC class I and 11 human leukocyte antigens and exhibit increased CD47 expression. In some embodiments, the pluripotent stem cell and any cell differentiated from such a pluripotent stem cell is modified to exhibit reduced expression of MHC class I and II human leukocyte antigens and TCR complexes and exhibit increased CD47 expression.

In some embodiments, the pluripotent stem cell and any cell differentiated from such a pluripotent stem cell is modified to exhibit reduced expression of MHC class I and/or II human leukocyte antigens, to exhibit increased CD47 expression, and to exogenously express a chimeric antigen receptor. In some instances, the cell overexpresses CD47 polypeptides by harboring one or more CD47 transgenes. In some instances, the cell overexpresses CAR polypeptides by harboring one or more CAR transgenes. In some embodiments, the pluripotent stem cell and any cell differentiated from such a pluripotent stem cell is modified to exhibit reduced expression of MHC class I and II human leukocyte antigens, exhibit increased CD47 expression, and to exogenously express a chimeric antigen receptor. In some embodiments, the pluripotent stem cell and any cell differentiated from such a pluripotent stem cell is modified to exhibit reduced expression of MHC class I and II human leukocyte antigens and TCR complexes, to exhibit increased CD47 expression, and to exogenously express a chimeric antigen receptor.

Such pluripotent stem cells are hypoimmunogenic stem cells. Such differentiated cells are hypoimmunogenic cells.

Any of the pluripotent stem cells described herein can be differentiated into any cells of an organism and tissue. In some embodiments, the cells exhibit reduced expression of MHC class I and/or II human leukocyte antigens and reduced expression of TCR complexes. In some instances, expression of MHC class I and/or II human leukocyte antigens is reduced compared to unmodified or wild-type cell of the same cell type. In some instances, expression of TCR complexes is reduced compared to unmodified or wild-type cell of the same cell type. In some embodiments, the cells exhibit increased CD47 expression. In some instances, expression of CD47 is increased in cells encompassed by the present disclosure as compared to unmodified or wild-type cells of the same cell type. In some embodiments, the cells exhibit exogenous CAR expression. Methods for reducing levels of MHC class I and/or II human leukocyte antigens and TCR complexes and increasing the expression of CD47 and CARs are described herein.

In some embodiments, the cells used in the methods described herein evade immune recognition and responses when administered to a patient (e.g., recipient subject). The cells can evade killing by immune cells in vitro and in vivo. In some embodiments, the cells evade killing by macrophages and NK cells. In some embodiments, the cells are ignored by immune cells or a subject's immune system. In other words, the cells administered in accordance with the methods described herein are not detectable by immune cells of the immune system. In some embodiments, the cells are cloaked and therefore avoid immune rejection.

Methods of determining whether a pluripotent stem cell and any cell differentiated from such a pluripotent stem cell evades immune recognition include, but are not limited to, IFN-γ Elispot assays, microglia killing assays, cell engraftment animal models, cytokine release assays, ELISAs, killing assays using bioluminescence imaging or chromium release assay or a real-time, quantitative microelectronic biosensor system for cell analysis (xCELLigence® RTCA system, Agilent), mixed-lymphocyte reactions, immunofluorescence analysis, etc.

Therapeutic cells outlined herein are useful to treat a disorder such as, but not limited to, a cancer, a genetic disorder, a chronic infectious disease, an autoimmune disorder, a neurological disorder, and the like.

1. T Lymphocytes Differentiated from Hypoimmunogenic Pluripotent Cells

Provided herein, T lymphocytes (T cells, including primary T cells) are derived from the HIP cells described herein (e.g., hypoimmunogenic iPSCs). Methods for generating T cells, including CAR-T cells, from pluripotent stem cells (e.g., iPSCs) are described, for example, in Iriguchi et al., Nature Communications 12, 430 (2021); Themeli et al., Cell Stem Cell, 16(4):357-366 (2015); Themeli et al., Nature Biotechnology 31:928-933 (2013).

T lymphocyte derived hypoimmunogenic cells include, but are not limited to, primary T cells that evade immune recognition. In some embodiments, the engineered CAR-T cells are produced (e.g., generated, cultured, or derived) from T cells such as primary T cells. In some instances, primary T cells are obtained (e.g., harvested, extracted, removed, or taken) from a subject or an individual. In some embodiments, primary T cells are produced from a pool of T cells such that the T cells are from one or more subjects (e.g., one or more human including one or more healthy humans). In some embodiments, the pool of primary T cells is from 1-100, 1-50, 1-20, 1-10, 1 or more, 2 or more, 3 or more, 4 or more, 5 or more, 10 or more, 20 or more, 30 or more, 40 or more, 50 or more, or 100 or more subjects. In some embodiments, the donor subject is different from the patient (e.g., the recipient that is administered the therapeutic cells). In some embodiments, the pool of T cells does not include cells from the patient. In some embodiments, one or more of the donor subjects from which the pool of T cells is obtained are different from the patient.

In some embodiments, the engineered CAR-T cells do not activate an immune response in the patient (e.g., recipient upon administration). Provided are methods of treating a disorder by administering a population of hypoimmunogenic cells to a subject (e.g., recipient) or patient in need thereof. In some embodiments, the engineered CAR-T cells described herein comprise T cells engineered (e.g., are modified) to express a chimeric antigen receptor including but not limited to a chimeric antigen receptor described herein. In some instances, the T cells are populations or subpopulations of primary T cells from one or more individuals. In some embodiments, the T cells described herein such as the engineered or modified T cells comprise reduced expression of an endogenous T cell receptor.

In some embodiments, the HIP-derived T cell includes a chimeric antigen receptor (CAR). Any suitable CAR can be included in the hyHIP-derived T cell, including the CARs described herein. In some embodiments, the hypoimmunogenic induced pluripotent stem cell-derived T cell includes a polynucleotide encoding a CAR, wherein the polynucleotide is inserted in a genomic locus. In some embodiments, the polynucleotide is inserted into a safe harbor or target locus. In some embodiments, the polynucleotide is inserted in a B2M, CIITA, TRAC, TRB, PD-1 or CTLA-4 gene. Any suitable method can be used to insert the CAR into the genomic locus of the hypoimmunogenic cell including the gene editing methods described herein (e.g., a CRISPR/Cas system).

HIP-derived T cells provided herein are useful for the treatment of suitable cancers including, but not limited to, lymphoma, leukemia, B cell acute lymphoblastic leukemia (B-ALL), diffuse large B-cell lymphoma, B-cell Non-Hodgkin lymphoma (B-NHL), B-cell chronic lymphoblastic leukemia, liver cancer, pancreatic cancer, breast cancer, ovarian cancer, colorectal cancer, lung cancer, non-small cell lung cancer, acute myeloid lymphoid leukemia, multiple myeloma, gastric cancer, gastric adenocarcinoma, pancreatic adenocarcinoma, glioblastoma, neuroblastoma, lung squamous cell carcinoma, hepatocellular carcinoma, and bladder cancer. In some embodiments, any of the exemplary cancers are also a CD19-negative cancer, a CD22-positive cancer, a CD19-negative/CD22-positive cancer, or a CD19-positive cancer. In certain embodiments, any of the exemplary cancers underwent antigen evasion and no longer express an antigen or have reduced expression of an antigen previously expressed. For example, any of the exemplary cancers can be a CD19-negative and a CD22-positive cancer but were previously CD19-positive and CD22-negative or CD22-positive.

2. NK Cells Derived from Hypoimmunogenic Pluripotent Cells

Provided herein, natural killer (NK) cells are derived from the HIP cells described herein (e.g., hypoimmunogenic iPSCs).

NK cells (also defined as ‘large granular lymphocytes’) represent a cell lineage differentiated from the common lymphoid progenitor (which also gives rise to B lymphocytes and T lymphocytes). Unlike T-cells, NK cells do not naturally comprise CD3 at the plasma membrane. Importantly, NK cells do not express a TCR and typically also lack other antigen-specific cell surface receptors (as well as TCRs and CD3, they also do not express immunoglobulin B-cell receptors, and instead typically express CD16 and CD56). NK cell cytotoxic activity does not require sensitization but is enhanced by activation with a variety of cytokines including IL-2. NK cells are generally thought to lack appropriate or complete signaling pathways necessary for antigen-receptor-mediated signaling, and thus are not thought to be capable of antigen receptor-dependent signaling, activation and expansion. NK cells are cytotoxic, and balance activating and inhibitory receptor signaling to modulate their cytotoxic activity. For instance, NK cells expressing CD16 may bind to the Fc domain of antibodies bound to an infected cell, resulting in NK cell activation. By contrast, activity is reduced against cells expressing high levels of MHC class I proteins. On contact with a target cell NK cells release proteins such as perforin, and enzymes such as proteases (granzymes). Perforin can form pores in the cell membrane of a target cell, inducing apoptosis or cell lysis.

There are a number of techniques that can be used to generate NK cells, including CAR-NK-cells, from pluripotent stem cells (e.g., iPSC); see, for example, Zhu et al., Methods Mol Biol. 2019; 2048:107-119; Knorr et al., Stem Cells Transl Med. 2013 2(4):274-83. doi: 10.5966/sctm.2012-0084; Zeng et al., Stem Cell Reports. 2017 Dec. 12; 9(6):1796-1812; Ni et al., Methods Mol Biol. 2013; 1029:33-41; Bernareggi et al., Exp Hematol. 2019 71:13-23; Shankar et al., Stem Cell Res Ther. 2020; 11(1):234, all of which are incorporated herein by reference in their entirety and specifically for the methodologies and reagents for differentiation. Differentiation can be assayed as is known in the art, generally by evaluating the presence of NK cell associated and/or specific markers, including, but not limited to, CD56, KIRs, CD16, NKp44, NKp46, NKG2D, TRAIL, CD122, CD27, CD244, NK1.1, NKG2A/C, NCR1, Ly49, CD49b, CD11b, KLRG1, CD43, CD62L, and/or CD226.

In some embodiments, the hypoimmunogenic pluripotent cells are differentiated into hepatocytes to address loss of the hepatocyte functioning or cirrhosis of the liver. There are a number of techniques that can be used to differentiate HIP cells into hepatocytes; see for example, Pettinato et al., doi: 10.1038/spre32888, Snykers et al., Methods Mol Biol., 2011 698:305-314, Si-Tayeb et al., Hepatology, 2010, 51:297-305 and Asgari et al., Stem Cell Rev., 2013, 9(4):493-504, all of which are incorporated herein by reference in their entirety and specifically for the methodologies and reagents for differentiation. Differentiation can be assayed as is known in the art, generally by evaluating the presence of hepatocyte associated and/or specific markers, including, but not limited to, albumin, alpha fetoprotein, and fibrinogen. Differentiation can also be measured functionally, such as the metabolization of ammonia, LDL storage and uptake, ICG uptake and release, and glycogen storage.

In some embodiments, the NK cells do not activate an innate and/or an adaptive immune response in the patient (e.g., recipient upon administration). Provided are methods of treating a disorder by administering a population of NK cells to a subject (e.g., recipient) or patient in need thereof. In some embodiments, the NK cells described herein comprise NK cells engineered (e.g., are modified) to express a chimeric antigen receptor including but not limited to a chimeric antigen receptor described herein. Any suitable CAR can be included in the NK cells, including the CARs described herein. In some embodiments, the NK cell includes a polynucleotide encoding a CAR, wherein the polynucleotide is inserted in a genomic locus. In some embodiments, the polynucleotide is inserted into a safe harbor or a target locus. In some embodiments, the polynucleotide is inserted in a B2M, CIITA, PD1 or CTLA4 gene. Any suitable method can be used to insert the CAR into the genomic locus of the NK cell including the gene editing methods described herein (e.g., a CRISPR/Cas system).

### J. Gene Editing Systems

In some aspects, the one or more polynucleotides (e.g., transgenes) encoding one or more tolerogenic factors can be integrated into the genome of a host cell (e.g., an allogeneic donor cell) using certain methods and compositions disclosed herein.

### 1. Vectors

In some embodiments, a vector herein is a nucleic acid molecule capable of transferring or transporting another nucleic acid molecule, including into the cell or into the genome of a cell. The transferred nucleic acid is generally linked to, e.g., inserted into, the vector nucleic acid molecule. A vector may include sequences that direct autonomous replication in a cell or may include sequences sufficient to allow integration into host cell DNA. Useful vectors include, for example, plasmids (e.g., DNA plasmids or RNA plasmids), transposons, cosmids, bacterial artificial chromosomes, and viral vectors. Useful viral vectors include, e.g., replication defective retroviruses and lentiviruses. Non-viral vectors may require a delivery vehicle to facilitate entry of the nucleic acid molecule into a cell.

A viral vector can comprise a nucleic acid molecule that includes virus-derived nucleic acid elements that typically facilitate transfer of the nucleic acid molecule or integration into the genome of a cell or to a viral particle that mediates nucleic acid transfer. Viral particles typically include various viral components and sometimes also host cell components in addition to nucleic acid(s). A viral vector can comprise, e.g., a virus or viral particle capable of transferring a nucleic acid into a cell, or to the transferred nucleic acid (e.g., as naked DNA). Viral vectors and transfer plasmids can comprise structural and/or functional genetic elements that are primarily derived from a virus. A retroviral vector can comprise a viral vector or plasmid containing structural and functional genetic elements, or portions thereof, that are primarily derived from a retrovirus.

In some vectors disclosed herein, at least part of one or more protein coding regions that contribute to or are essential for replication may be absent compared to the corresponding wild-type virus. This makes the viral vector replication-defective. In some embodiments, the vector is capable of transducing a target non-dividing host cell and/or integrating its genome into a host genome.

In some embodiments, the retroviral nucleic acid comprises one or more of or all of: a 5′ promoter (e.g., to control expression of the entire packaged RNA), a 5′ LTR (e.g., that includes R (polyadenylation tail signal) and/or U5 which includes a primer activation signal), a primer binding site, a psi packaging signal, a RRE element for nuclear export, a promoter directly upstream of the transgene to control transgene expression, a transgene (or other exogenous agent element), a polypurine tract, and a 3′ LTR (e.g., that includes a mutated U3, a R, and U5). In some embodiments, the retroviral nucleic acid further comprises one or more of a cPPT, a WPRE, and/or an insulator element.

A retrovirus typically replicates by reverse transcription of its genomic RNA into a linear double-stranded DNA copy and subsequently covalently integrates its genomic DNA into a host genome. The structure of a wild-type retrovirus genome often comprises a 5′ long terminal repeat (LTR) and a 3′ LTR, between or within which are located a packaging signal to enable the genome to be packaged, a primer binding site, integration sites to enable integration into a host cell genome and gag, pol and env genes encoding the packaging components which promote the assembly of viral particles. More complex retroviruses have additional features, such as rev and RRE sequences in HIV, which enable the efficient export of RNA transcripts of the integrated provirus from the nucleus to the cytoplasm of an infected target cell. In the provirus, the viral genes are flanked at both ends by regions called long terminal repeats (LTRs). The LTRs are involved in proviral integration and transcription. LTRs also serve as enhancer-promoter sequences and can control the expression of the viral genes. Encapsidation of the retroviral RNAs occurs by virtue of a psi sequence located at the 5′ end of the viral genome.

The LTRs themselves are typically similar (e.g., identical) sequences that can be divided into three elements, which are called U3, R and U5. U3 is derived from the sequence unique to the 3′ end of the RNA. R is derived from a sequence repeated at both ends of the RNA and U5 is derived from the sequence unique to the 5′ end of the RNA. The sizes of the three elements can vary considerably among different retroviruses.

For the viral genome, the site of transcription initiation is typically at the boundary between U3 and R in one LTR and the site of poly (A) addition (termination) is at the boundary between R and U5 in the other LTR. U3 contains most of the transcriptional control elements of the provirus, which include the promoter and multiple enhancer sequences responsive to cellular and in some cases, viral transcriptional activator proteins. Some retroviruses comprise any one or more of the following genes that code for proteins that are involved in the regulation of gene expression: tot, rev, tax and rex.

With regard to the structural genes gag, pol and env themselves, gag encodes the internal structural protein of the virus. Gag protein is proteolytically processed into the mature proteins MA (matrix), CA (capsid) and NC (nucleocapsid). The pol gene encodes the reverse transcriptase (RT), which contains DNA polymerase, associated RNase H and integrase (IN), which mediate replication of the genome. The env gene encodes the surface (SU) glycoprotein and the transmembrane (TM) protein of the virion, which form a complex that interacts specifically with cellular receptor proteins. This interaction promotes infection, e.g., by fusion of the viral membrane with the cell membrane.

In a replication-defective retroviral vector genome gag, pol and env may be absent or not functional. The R regions at both ends of the RNA are typically repeated sequences. U5 and U3 represent unique sequences at the 5′ and 3′ ends of the RNA genome respectively. Retroviruses may also contain additional genes which code for proteins other than gag, pol and env. Examples of additional genes include (in HIV), one or more of vif, vpr, vpx, vpu, tat, rev and nef. EIAV has (amongst others) the additional gene S2.

Illustrative retroviruses suitable for use in particular embodiments, include, but are not limited to: Moloney murine leukemia virus (M-MuLV), Moloney murine sarcoma virus (MoMSV), Harvey murine sarcoma virus (HaMuSV), murine mammary tumor virus (MuMTV), gibbon ape leukemia virus (GaLV), feline leukemia virus (FLV), spumavirus, Friend murine leukemia virus, Murine Stem Cell Virus (MSCV) and Rous Sarcoma Virus (RSV)) and lentivirus.

In some embodiments the retrovirus is a Gammretrovirus. In some embodiments the retrovirus is an Epsilonretrovirus. In some embodiments the retrovirus is an Alpharetrovirus. In some embodiments the retrovirus is a Betaretrovirus. In some embodiments the retrovirus is a Deltaretrovirus. In some embodiments the retrovirus is a Spumaretrovirus. In some embodiments the retrovirus is an endogenous retrovirus. In some embodiments the retrovirus is a lentivirus.

In some embodiments, a retroviral or lentivirus vector further comprises one or more insulator elements, e.g., an insulator element disclosed herein. In various embodiments, the vectors comprise a promoter operably linked to a polynucleotide encoding an exogenous agent. The vectors may have one or more LTRs, wherein either LTR comprises one or more modifications, such as one or more nucleotide substitutions, additions, or deletions. The vectors may further comprise one of more accessory elements to increase transduction efficiency (e.g., a cPPT/FLAP), viral packaging (e.g., a Psi (Y) packaging signal, RRE), and/or other elements that increase exogenous gene expression (e.g., poly (A) sequences), and may optionally comprise a WPRE or HPRE. In some embodiments, a lentiviral nucleic acid comprises one or more of, e.g., all of, e.g., from 5′ to 3′, a promoter (e.g., CMV), an R sequence (e.g., comprising TAR), a U5 sequence (e.g., for integration), a PBS sequence (e.g., for reverse transcription), a DIS sequence (e.g., for genome dimerization), a psi packaging signal, a partial gag sequence, an RRE sequence (e.g., for nuclear export), a cPPT sequence (e.g., for nuclear import), a promoter to drive expression of the exogenous agent, a gene encoding the exogenous agent, a WPRE sequence (e.g., for efficient transgene expression), a PPT sequence (e.g., for reverse transcription), an R sequence (e.g., for polyadenylation and termination), and a U5 signal (e.g., for integration).

Illustrative lentiviruses include but are not limited to: HIV (human immunodeficiency virus; including HIV type 1, and HIV type 2); visna-maedi virus (VMV) virus; the caprine arthritis-encephalitis virus (CAEV); equine infectious anemia virus (EIAV); feline immunodeficiency virus (FIV); bovine immune deficiency virus (BIV); and simian immunodeficiency virus (SIV). In some embodiments, HIV based vector backbones (i.e., HIV cis-acting sequence elements) are used. A lentivirus vector can comprise a viral vector or plasmid containing structural and functional genetic elements, or portions thereof, including LTRs that are primarily derived from a lentivirus.

In some embodiments, a lentivirus vector (e.g., lentiviral expression vector) may comprise a lentiviral transfer plasmid (e.g., as naked DNA) or an infectious lentiviral particle. With respect to elements such as cloning sites, promoters, regulatory elements, heterologous nucleic acids, etc., it is to be understood that the sequences of these elements can be present in RNA form in lentiviral particles and can be present in DNA form in DNA plasmids.

In some embodiments, a lentivirus vector is a vector with sufficient retroviral genetic information to allow packaging of an RNA genome, in the presence of packaging components, into a viral particle capable of infecting a target cell. Infection of the target cell can comprise reverse transcription and integration into the target cell genome. The RLV typically carries non-viral coding sequences which are to be delivered by the vector to the target cell. In some embodiments, an RLV is incapable of independent replication to produce infectious retroviral particles within the target cell. Usually the RLV lacks a functional gag-pol and/or env gene and/or other genes involved in replication. The vector may be configured as a split-intron vector, e.g., as disclosed in PCT patent application WO 99/15683, which is herein incorporated by reference in its entirety.

In some embodiments, the lentivirus vector comprises a minimal viral genome, e.g., the viral vector has been manipulated so as to remove the non-essential elements and to retain the essential elements in order to provide the required functionality to infect, transduce and deliver a nucleotide sequence of interest to a target host cell, e.g., as disclosed in WO 98/17815, which is herein incorporated by reference in its entirety.

A minimal lentiviral genome may comprise, e.g., (5′)R-U5-one or more first nucleotide sequences-U3-R(3′). However, the plasmid vector used to produce the lentiviral genome within a source cell can also include transcriptional regulatory control sequences operably linked to the lentiviral genome to direct transcription of the genome in a source cell. These regulatory sequences may comprise the natural sequences associated with the transcribed retroviral sequence, e.g., the 5′ U3 region, or they may comprise a heterologous promoter such as another viral promoter, for example the CMV promoter. Some lentiviral genomes comprise additional sequences to promote efficient virus production. For example, in the case of HIV, rev and RRE sequences may be included.

### 2. Recombinant Expression

For all of these technologies, well-known recombinant techniques are used, to generate recombinant nucleic acids as disclosed herein. In certain embodiments, the recombinant nucleic acids (e.g., polynucleotides encoding, e.g., one or more tolerogenic factors) may be operably linked to one or more regulatory nucleotide sequences in an expression construct. Regulatory nucleotide sequences are generally appropriate for the host cell and recipient subject to be treated. Numerous types of appropriate expression vectors and suitable regulatory sequences are known in the art for a variety of host cells. Typically, the one or more regulatory nucleotide sequences may include, but are not limited to, promoter sequences, leader or signal sequences, ribosomal binding sites, transcriptional start and termination sequences, translational start and termination sequences, and enhancer or activator sequences. Constitutive or inducible promoters as known in the art are also contemplated. The promoters may be either naturally occurring promoters, hybrid promoters that combine elements of more than one promoter, or synthetic promoters. An expression construct may be present in a cell on an episome, such as a plasmid, or the expression construct may be inserted in a chromosome such as in a gene locus. In some embodiment, the expression vector includes a selectable marker gene to allow the selection of transformed host cells. In some embodiments, an expression vector comprises a nucleotide sequence encoding a variant polypeptide operably linked to at least one regulatory sequence. Regulatory sequence for use herein include promoters, enhancers, and other expression control elements. In some embodiments, an expression vector is designed for the choice of the host cell to be transformed, the particular variant polypeptide desired to be expressed, the vector's copy number, the ability to control that copy number, and/or the expression of any other protein encoded by the vector, such as antibiotic markers.

Examples of suitable mammalian promoters include, for example, promoters from the following genes: elongation factor 1 alpha (EF1α) promoter, CAG promoter, ubiquitin/S27a promoter of the hamster (WO 97/15664), Simian vacuolating virus 40 (SV40) early promoter, adenovirus major late promoter, mouse metallothionein-I promoter, the long terminal repeat region of Rous Sarcoma Virus (RSV), mouse mammary tumor virus promoter (MMTV), Moloney murine leukemia virus Long Terminal repeat region, and the early promoter of human Cytomegalovirus (CMV). Examples of other heterologous mammalian promoters are the actin, immunoglobulin or heat shock promoter(s). In additional embodiments, promoters for use in mammalian host cells can be obtained from the genomes of viruses such as polyoma virus, fowlpox virus (UK 2,211,504 published 5 Jul. 1989), bovine papilloma virus, avian sarcoma virus, cytomegalovirus, a retrovirus, hepatitis-B virus and Simian Virus 40 (SV40). In further embodiments, heterologous mammalian promoters are used. Examples include the actin promoter, an immunoglobulin promoter, and heat-shock promoters. The early and late promoters of SV40 are conveniently obtained as an SV40 restriction fragment which also contains the SV40 viral origin of replication (Fiers et al., Nature 273: 113-120 (1978)). The immediate early promoter of the human cytomegalovirus is conveniently obtained as a Hindill restriction enzyme fragment (Greenaway et al., Gene 18: 355-360 (1982)). The foregoing references are incorporated by reference in their entirety.

In some embodiments, the expression vector is a bicistronic or multicistronic expression vector. Bicistronic or multicistronic expression vectors may include (1) multiple promoters fused to each of the open reading frames; (2) insertion of splicing signals between genes; (3) fusion of genes whose expressions are driven by a single promoter; and (4) insertion of proteolytic cleavage sites between genes (self-cleavage peptide) or insertion of internal ribosomal entry sites (IRESs) between genes.

The process of introducing the polynucleotides disclosed herein into cells can be achieved by any suitable technique. Suitable techniques include calcium phosphate or lipid-mediated transfection, electroporation, fusogens, and transduction or infection using a viral vector. In some embodiments, the polynucleotides are introduced into a cell via viral transduction (e.g., AAV transduction, lentiviral transduction) or otherwise delivered on a viral vector (e.g., fusogen-mediated delivery). In some of these embodiments, the AAV vector is an AAV6 vector or an AAV9 vector. Additional AAV vectors for gene delivery are disclosed in, for example, Wang et al., “Adeno-associated virus vector as a platform for gene therapy deliver,” Nature Reviews Drug Discovery 18: 358-378 (2019), the disclosure is incorporated herein by reference in its entirety. In some embodiments, the polynucleotides are introduced into a cell via a fusogen-mediated delivery or a transposase system selected from the group consisting of conditional or inducible transposases, conditional or inducible PiggyBac transposons, conditional or inducible Sleeping Beauty (SB11) transposons, conditional or inducible Mos1 transposons, and conditional or inducible Tol2 transposons.

In some embodiments, the cells provided herein are genetically modified to include one or more exogenous polynucleotides inserted into one or more genomic loci of the cell. In some embodiments, the exogenous polynucleotide encodes a protein of interest, e.g., a tolerogenic factor. Any suitable method can be used to insert the exogenous polynucleotide into the genomic locus of the cell including the gene editing methods disclosed herein (e.g., a CRISPR/Cas system). In some embodiments, the exogenous polynucleotide is inserted into at least one allele of the cell using viral transduction, for example, with a vector. In some embodiments, the vector is a pseudotyped, self-inactivating lentiviral vector that carries the exogenous polynucleotide. In some embodiments, the vector is a self-inactivating lentiviral vector pseudotyped with a vesicular stomatitis VSV-G envelope, and which carries the exogenous polynucleotide. In some embodiments, the exogenous polynucleotide is inserted into at least one allele of the cell using viral transduction. In some embodiments, the exogenous polynucleotide is inserted into at least one allele of the cell using a lentivirus based viral vector.

### 3. Site-Directed Insertion (Knock-In)

In some embodiments, the one or more transgenes encoding, e.g., one or more tolerogenic factors can be inserted into a specific genomic locus of a host cell (e.g., an allogeneic donor cell). A number of gene editing methods can be used to insert a transgene into a specific genomic locus of choice. Gene editing is a type of genetic engineering in which a nucleotide sequence may be inserted, deleted, modified, or replaced in the genome of a living organism.

In some embodiments, a rare-cutting endonuclease is introduced into a cell containing the target polynucleotide sequence in the form of a nucleic acid encoding a rare-cutting endonuclease. The process of introducing the nucleic acids into cells can be achieved by any suitable technique. Suitable techniques include calcium phosphate or lipid-mediated transfection, electroporation, and transduction or infection using a viral vector. In some embodiments, the nucleic acid comprises DNA. In some embodiments, the nucleic acid comprises a modified DNA, as disclosed herein. In some embodiments, the nucleic acid comprises an mRNA. In some embodiments, the nucleic acid comprises a modified mRNA, as disclosed herein (e.g., a synthetic, modified mRNA).

The present disclosure contemplates altering target polynucleotide sequences in any manner which is available to the skilled artisan utilizing a gene editing system (e.g., CRISPR/Cas) of the present disclosure. Any CRISPR/Cas system that is capable of altering a target polynucleotide sequence in a cell can be used. Such CRISPR-Cas systems can employ a variety of Cas proteins (Haft et al. PLoS Comput Biol. 2005; 1(6)e60). The molecular machinery of such Cas proteins that allows the CRISPR/Cas system to alter target polynucleotide sequences in cells include RNA binding proteins, endo- and exo-nucleases, helicases, and polymerases. In some embodiments, the CRISPR/Cas system is a CRISPR type I system. In some embodiments, the CRISPR/Cas system is a CRISPR type II system. In some embodiments, the CRISPR/Cas system is a CRISPR type V system.

The CRISPR/Cas systems of the present disclosure can be used to alter any target polynucleotide sequence in a cell. Those skilled in the art will readily appreciate that desirable target polynucleotide sequences to be altered in any particular cell may correspond to any genomic sequence for which expression of the genomic sequence is associated with a disorder or otherwise facilitates entry of a pathogen into the cell. For example, a desirable target polynucleotide sequence to alter in a cell may be a polynucleotide sequence corresponding to a genomic sequence which contains a disease associated single polynucleotide polymorphism. In such example, the CRISPR/Cas systems of the present disclosure can be used to correct the disease associated SNP in a cell by replacing it with a wild-type allele. As another example, a polynucleotide sequence of a target gene which is responsible for entry or proliferation of a pathogen into a cell may be a suitable target for deletion or insertion to disrupt the function of the target gene to prevent the pathogen from entering the cell or proliferating inside the cell.

In some embodiments, the target polynucleotide sequence is a genomic sequence. In some embodiments, the target polynucleotide sequence is a human genomic sequence. In some embodiments, the target polynucleotide sequence is a mammalian genomic sequence. In some embodiments, the target polynucleotide sequence is a vertebrate genomic sequence.

In some embodiments, a CRISPR/Cas system of the present disclosure includes a Cas protein and at least one to two ribonucleic acids that are capable of directing the Cas protein to and hybridizing to a target motif of a target polynucleotide sequence. As used herein, “protein” and “polypeptide” are used interchangeably to refer to a series of amino acid residues joined by peptide bonds (i.e., a polymer of amino acids) and include modified amino acids (e.g., phosphorylated, glycated, glycosylated, etc.) and amino acid analogs. Exemplary polypeptides or proteins include gene products, naturally occurring proteins, homologs, paralogs, fragments and other equivalents, variants, and analogs of the above.

In some embodiments, a Cas protein comprises one or more amino acid substitutions or modifications. In some embodiments, the one or more amino acid substitutions comprises a conservative amino acid substitution. In some instances, substitutions and/or modifications can prevent or reduce proteolytic degradation and/or extend the half-life of the polypeptide in a cell. In some embodiments, the Cas protein can comprise a peptide bond replacement (e.g., urea, thiourea, carbamate, sulfonyl urea, etc.). In some embodiments, the Cas protein can comprise a naturally occurring amino acid. In some embodiments, the Cas protein can comprise an alternative amino acid (e.g., D-amino acids, beta-amino acids, homocysteine, phosphoserine, etc.). In some embodiments, a Cas protein can comprise a modification to include a moiety (e.g., PEGylation, glycosylation, lipidation, acetylation, end-capping, etc.).

In some embodiments, a Cas protein comprises a core Cas protein, isoform thereof, or any Cas-like protein with similar function or activity of any Cas protein or isoform thereof. In some embodiments, a Cas protein comprises a core Cas protein. Exemplary Cas core proteins include, but are not limited to Cas1, Cas2, Cas3, Cas4, Cas5, Cas6, Cas7, Cas8 and Cas9. In some embodiments, a Cas protein comprises type V Cas protein. In some embodiments, a Cas protein comprises a Cas protein of an E. coli subtype (also known as CASS2). Exemplary Cas proteins of the E. Coli subtype include, but are not limited to Cse1, Cse2, Cse3, Cse4, and Cas5e. In some embodiments, a Cas protein comprises a Cas protein of the Ypest subtype (also known as CASS3). Exemplary Cas proteins of the Ypest subtype include, but are not limited to Csy1, Csy2, Csy3, and Csy4. In some embodiments, a Cas protein comprises a Cas protein of the Nmeni subtype (also known as CASS4). Exemplary Cas proteins of the Nmeni subtype include, but are not limited to Csn1 and Csn2. In some embodiments, a Cas protein comprises a Cas protein of the Dvulg subtype (also known as CASS1). Exemplary Cas proteins of the Dvulg subtype include Csd1, Csd2, and Cas5d. In some embodiments, a Cas protein comprises a Cas protein of the Tneap subtype (also known as CASS7). Exemplary Cas proteins of the Tneap subtype include, but are not limited to, Cst1, Cst2, Cas5t. In some embodiments, a Cas protein comprises a Cas protein of the Hmari subtype. Exemplary Cas proteins of the Hmari subtype include, but are not limited to Csh1, Csh2, and Cas5h. In some embodiments, a Cas protein comprises a Cas protein of the Apern subtype (also known as CASS5). Exemplary Cas proteins of the Apern subtype include, but are not limited to Csa1, Csa2, Csa3, Csa4, Csa5, and Cas5a. In some embodiments, a Cas protein comprises a Cas protein of the Mtube subtype (also known as CASS6). Exemplary Cas proteins of the Mtube subtype include, but are not limited to Csm1, Csm2, Csm3, Csm4, and Csm5. In some embodiments, a Cas protein comprises a RAMP module Cas protein. Exemplary RAMP module Cas proteins include, but are not limited to, Cmr1, Cmr2, Cmr3, Cmr4, Cmr5, and Cmr6. See, e.g., Klompe et al., Nature 571, 219-225 (2019); Strecker et al., Science 365, 48-53 (2019). Examples of Cas proteins include, but are not limited to: Cas3, Cas8a, Cas5, Cas8b, Cas8c, Cas10d, Cse1, Cse2, Csy1, Csy2, Csy3, and/or GSU0054. In some embodiments, a Cas protein comprises Cas3, Cas8a, Cas5, Cas8b, Cas8c, Cas10d, Cse1, Cse2, Csy1, Csy2, Csy3, and/or GSU0054. Examples of Cas proteins include, but are not limited to: Cas9, Csn2, and/or Cas4. In some embodiments, a Cas protein comprises Cas9, Csn2, and/or Cas4. In some embodiments, examples of Cas proteins include, but are not limited to: Cas10, Csm2, Cmr5, Cas10, Csx11, and/or Csx10. In some embodiments, a Cas protein comprises a Cas10, Csm2, Cmr5, Cas10, Csx11, and/or Csx10. In some embodiments, examples of Cas proteins include, but are not limited to: Csf1. In some embodiments, a Cas protein comprises Csf1.ln some embodiments, examples of Cas proteins include, but are not limited to: Cas12a, Cas12b, Cas12c, C2c4, C2c8, C2c5, C2c10, and C2c9; as well as CasX (Cas12e) and CasY (Cas12d). Also see, e.g., Koonin et al., Curr Opin Microbiol. 2017; 37:67-78: “Diversity, classification and evolution of CRISPR-Cas systems.” In some embodiments, a Cas protein comprises Cas12a, Cas12b, Cas12c, Cas12d, Cas12e, Cas12d, and/or Cas12e. In some embodiments, a Cas protein comprises Cas13, Cas13a, C2c2, Cas13b, Cas13c, and/or Cas13d. In some embodiments, the CRISPR/Cas system comprises a Cas effector protein selected from the group consisting of: a) Cas3, Cas8a, Cas5, Cas8b, Cas8c, Cas10d, Cse1, Cse2, Csy1, Csy2, Csy3, and GSU0054; b) Cas9, Csn2, and Cas4; c) Cas10, Csm2, Cmr5, Cas10, Csx11, and Csx10; d) Csf1; e) Cas12a, Cas12b, Cas12c, C2c4, C2c8, C2c5, C2c10, C2c9, CasX (Cas12e), and CasY (Cas12d); and f) Cas13, Cas13a, C2c2, Cas13b, Cas13c, and Cas13d.

In some embodiments, a Cas protein comprises any one of the Cas proteins disclosed herein or a functional portion thereof. As used herein, “functional portion” refers to a portion of a peptide which retains its ability to complex with at least one ribonucleic acid (e.g., guide RNA (gRNA)) and cleave a target polynucleotide sequence. In some embodiments, the functional portion comprises a combination of operably linked Cas9 protein functional domains selected from the group consisting of a DNA binding domain, at least one RNA binding domain, a helicase domain, and an endonuclease domain. In some embodiments, the functional portion comprises a combination of operably linked Cas12a (also known as Cpf1) protein functional domains selected from the group consisting of a DNA binding domain, at least one RNA binding domain, a helicase domain, and an endonuclease domain. In some embodiments, the functional domains form a complex. In some embodiments, a functional portion of the Cas9 protein comprises a functional portion of a RuvC-like domain. In some embodiments, a functional portion of the Cas9 protein comprises a functional portion of the HNH nuclease domain. In some embodiments, a functional portion of the Cas12a protein comprises a functional portion of a RuvC-like domain.

In some embodiments, exogenous Cas protein can be introduced into the cell in polypeptide form. In certain embodiments, Cas proteins can be conjugated to or fused to a cell-penetrating polypeptide or cell-penetrating peptide. As used herein, “cell-penetrating polypeptide” and “cell-penetrating peptide” refers to a polypeptide or peptide, respectively, which facilitates the uptake of molecule into a cell. The cell-penetrating polypeptides can contain a detectable label.

In many embodiments, Cas proteins can be conjugated to or fused to a charged protein (e.g., that carries a positive, negative or overall neutral electric charge). Such linkage may be covalent. In some embodiments, the Cas protein can be fused to a superpositively charged GFP to significantly increase the ability of the Cas protein to penetrate a cell (Cronican et al. ACS Chem Biol. 2010; 5(8):747-52). In certain embodiments, the Cas protein can be fused to a protein transduction domain (PTD) to facilitate its entry into a cell. Exemplary PTDs include Tat, oligoarginine, and penetratin. In some embodiments, the Cas9 protein comprises a Cas9 polypeptide fused to a cell-penetrating peptide. In some embodiments, the Cas9 protein comprises a Cas9 polypeptide fused to a PTD. In some embodiments, the Cas9 protein comprises a Cas9 polypeptide fused to a tat domain. In some embodiments, the Cas9 protein comprises a Cas9 polypeptide fused to an oligoarginine domain. In some embodiments, the Cas9 protein comprises a Cas9 polypeptide fused to a penetratin domain. In some embodiments, the Cas9 protein comprises a Cas9 polypeptide fused to a superpositively charged GFP. In some embodiments, the Cas12a protein comprises a Cas12a polypeptide fused to a cell-penetrating peptide. In some embodiments, the Cas12a protein comprises a Cas12a polypeptide fused to a PTD. In some embodiments, the Cas12a protein comprises a Cas12a polypeptide fused to a tat domain. In some embodiments, the Cas12a protein comprises a Cas12a polypeptide fused to an oligoarginine domain. In some embodiments, the Cas12a protein comprises a Cas12a polypeptide fused to a penetratin domain. In some embodiments, the Cas12a protein comprises a Cas12a polypeptide fused to a superpositively charged GFP.

In some embodiments, the Cas protein can be introduced into a cell containing the target polynucleotide sequence in the form of a nucleic acid encoding the Cas protein. The process of introducing the nucleic acids into cells can be achieved by any suitable technique. Suitable techniques include calcium phosphate or lipid-mediated transfection, electroporation, and transduction or infection using a viral vector. In some embodiments, the nucleic acid comprises DNA. In some embodiments, the nucleic acid comprises a modified DNA, as disclosed herein. In some embodiments, the nucleic acid comprises mRNA. In some embodiments, the nucleic acid comprises a modified mRNA, as disclosed herein (e.g., a synthetic, modified mRNA).

In some embodiments, the Cas protein is complexed with one to two ribonucleic acids. In some embodiments, the Cas protein is complexed with two ribonucleic acids. In some embodiments, the Cas protein is complexed with one ribonucleic acid. In some embodiments, the Cas protein is encoded by a modified nucleic acid, as disclosed herein (e.g., a synthetic, modified mRNA).

The methods of the present disclosure contemplate the use of any ribonucleic acid that is capable of directing a Cas protein to and hybridizing to a target motif of a target polynucleotide sequence. In some embodiments, at least one of the ribonucleic acids comprises tracrRNA. In some embodiments, at least one of the ribonucleic acids comprises CRISPR RNA (crRNA). In some embodiments, a single ribonucleic acid comprises a guide RNA that directs the Cas protein to and hybridizes to a target motif of the target polynucleotide sequence in a cell. In some embodiments, at least one of the ribonucleic acids comprises a guide RNA that directs the Cas protein to and hybridizes to a target motif of the target polynucleotide sequence in a cell. In some embodiments, both of the one to two ribonucleic acids comprise a guide RNA that directs the Cas protein to and hybridizes to a target motif of the target polynucleotide sequence in a cell. The ribonucleic acids of the present disclosure can be selected to hybridize to a variety of different target motifs, depending on the particular CRISPR/Cas system employed, and the sequence of the target polynucleotide, as will be appreciated by those skilled in the art. The one to two ribonucleic acids can also be selected to minimize hybridization with nucleic acid sequences other than the target polynucleotide sequence. In some embodiments, the one to two ribonucleic acids hybridize to a target motif that contains at least two mismatches when compared with all other genomic nucleotide sequences in the cell. In some embodiments, the one to two ribonucleic acids hybridize to a target motif that contains at least one mismatch when compared with all other genomic nucleotide sequences in the cell. In some embodiments, the one to two ribonucleic acids are designed to hybridize to a target motif immediately adjacent to a deoxyribonucleic acid motif recognized by the Cas protein. In some embodiments, each of the one to two ribonucleic acids are designed to hybridize to target motifs immediately adjacent to deoxyribonucleic acid motifs recognized by the Cas protein which flank a mutant allele located between the target motifs.

In some embodiments, each of the one to two ribonucleic acids comprises guide RNAs that directs the Cas protein to and hybridizes to a target motif of the target polynucleotide sequence in a cell.

In some embodiments, one or two ribonucleic acids (e.g., guide RNAs) are complementary to and/or hybridize to sequences on the same strand of a target polynucleotide sequence. In some embodiments, one or two ribonucleic acids (e.g., guide RNAs) are complementary to and/or hybridize to sequences on the opposite strands of a target polynucleotide sequence. In some embodiments, the one or two ribonucleic acids (e.g., guide RNAs) are not complementary to and/or do not hybridize to sequences on the opposite strands of a target polynucleotide sequence. In some embodiments, the one or two ribonucleic acids (e.g., guide RNAs) are complementary to and/or hybridize to overlapping target motifs of a target polynucleotide sequence. In some embodiments, the one or two ribonucleic acids (e.g., guide RNAs) are complementary to and/or hybridize to offset target motifs of a target polynucleotide sequence.

In some embodiments, nucleic acids encoding Cas protein and nucleic acids encoding the at least one to two ribonucleic acids are introduced into a cell via viral transduction (e.g., lentiviral transduction). In some embodiments, the Cas protein is complexed with 1-2 ribonucleic acids. In some embodiments, the Cas protein is complexed with two ribonucleic acids. In some embodiments, the Cas protein is complexed with one ribonucleic acid. In some embodiments, the Cas protein is encoded by a modified nucleic acid, as disclosed herein (e.g., a synthetic, modified mRNA).

Exemplary gRNA sequences useful for CRISPR/Cas-based targeting of genes disclosed herein are provided in Table 35. The sequences can be found in WO2016183041 filed May 9, 2016, the disclosure including the Tables, Appendices, and Sequence Listing is incorporated herein by reference in its entirety.

Other exemplary gRNA sequences useful for CRISPR/Cas-based targeting of genes disclosed herein are provided in U.S. Provisional Patent Application No. 63/190,685, filed May 19, 2021, and in U.S. Provisional Patent Application No. 63/221,887, filed Jul. 14, 2021, the disclosures of which, including the Tables, Appendices, and Sequence Listings, are incorporated herein by reference in their entireties.

In some embodiments, the cells of the technology are made using Transcription Activator-Like Effector Nucleases (TALEN) methodologies. TALEN is a fusion protein consisting of a nucleic acid-binding domain typically derived from a Transcription Activator Like Effector (TALE) and one nuclease catalytic domain to cleave a nucleic acid target sequence. The catalytic domain is preferably a nuclease domain and more preferably a domain having endonuclease activity, like for instance I-Tevl, CoIE7, NucA and Fok-I. In numerous embodiments, the TALE domain can be fused to a meganuclease like for instance I-Crel and I-Onul or functional variant thereof. In a more preferred embodiment, said nuclease is a monomeric TALE-Nuclease. A™ monomeric TALE-Nuclease is a TALE-Nuclease that does not require dimerization for specific recognition and cleavage, such as the fusions of engineered TAL repeats with the catalytic domain of I-TevI disclosed in WO2012138927. TALEs are proteins from the bacterial species Xanthomonas comprise a plurality of repeated sequences, each repeat comprising di-residues in position 12 and 13 (RVD) that are specific to each nucleotide base of the nucleic acid targeted sequence. Binding domains with similar modular base-per-base nucleic acid binding properties (MBBBD) can also be derived from new modular proteins recently discovered by the applicant in a different bacterial species. The new modular proteins have the advantage of displaying more sequence variability than TAL repeats. Preferably, RVDs associated with recognition of the different nucleotides are HD for recognizing C, NG for recognizing T, NI for recognizing A, NN for recognizing G or A, NS for recognizing A, C, G or T, HG for recognizing T, IG for recognizing T, NK for recognizing G, HA for recognizing C, ND for recognizing C, HI for recognizing C, HN for recognizing G, NA for recognizing G, SN for recognizing G or A and YG for recognizing T, TL for recognizing A, VT for recognizing A or G and SW for recognizing A. In another embodiment, critical amino acids 12 and 13 can be mutated towards other amino acid residues in order to modulate their specificity towards nucleotides A, T, C and G and in particular to enhance this specificity. TALEN kits are sold commercially.

In some embodiments, the cells are manipulated using zinc finger nuclease (ZFN). A “zinc finger binding protein” is a protein or polypeptide that binds DNA, RNA and/or protein, preferably in a sequence-specific manner, as a result of stabilization of protein structure through coordination of a zinc ion. The term zinc finger binding protein is often abbreviated as zinc finger protein or ZFP. The individual DNA binding domains are typically referred to as “fingers.” A ZFP has least one finger, typically two fingers, three fingers, or six fingers. Each finger binds from two to four base pairs of DNA, typically three or four base pairs of DNA. A ZFP binds to a nucleic acid sequence called a target site or target segment. Each finger typically comprises an approximately 30 amino acid, zinc-chelating, DNA-binding subdomain. Studies have demonstrated that a single zinc finger of this class consists of an alpha helix containing the two invariant histidine residues coordinated with zinc along with the two cysteine residues of a single beta turn (see, e.g., Berg & Shi, Science 271:1081-1085 (1996)).

In some embodiments, the cells of the present disclosure are made using a homing endonuclease. Such homing endonucleases are well-known to the art (Stoddard 2005). Homing endonucleases recognize a DNA target sequence and generate a single- or double-strand break. Homing endonucleases are highly specific, recognizing DNA target sites ranging from 12 to 45 base pairs (bp) in length, usually ranging from 14 to 40 bp in length. The homing endonuclease according to the technology may for example correspond to a LAGLIDADG endonuclease, to a HNH endonuclease, or to a GIY-YIG endonuclease. Preferred homing endonuclease according to the present disclosure can be an I-Crel variant.

In some embodiments, the cells of the technology are made using a meganuclease. Meganucleases are by definition sequence-specific endonucleases recognizing large sequences (Chevalier, B. S. and B. L. Stoddard, Nucleic Acids Res., 2001, 29, 3757-3774). They can cleave unique sites in living cells, thereby enhancing gene targeting by 1000-fold or more in the vicinity of the cleavage site (Puchta et al., Nucleic Acids Res., 1993, 21, 5034-5040; Rouet et al., Mol. Cell. Biol., 1994, 14, 8096-8106; Choulika et al., Mol. Cell. Biol., 1995, 15, 1968-1973; Puchta et al., Proc. Natl. Acad. Sci. USA, 1996, 93, 5055-5060; Sargent et al., Mol. Cell. Biol., 1997, 17, 267-77; Donoho et al., Mol. Cell. Biol, 1998, 18, 4070-4078; Elliott et al., Mol. Cell. Biol., 1998, 18, 93-101; Cohen-Tannoudji et al., Mol. Cell. Biol., 1998, 18, 1444-1448).

Current gene editing techniques generally utilize the innate mechanism for cells to repair double-strand breaks (DSBs) in DNA. Eukaryotic cells repair DSBs by two primary repair pathways: non-homologous end-joining (NHEJ) and homology-directed repair (HDR). HDR typically occurs during late S phase or G2 phase, when a sister chromatid is available to serve as a repair template. NHEJ is more common and can occur during any phase of the cell cycle, but it is more error prone. In gene editing, NHEJ is generally used to produce insertion/deletion mutations (indels), which can produce targeted loss of function in a target gene by shifting the open reading frame (ORF) and producing alterations in the coding region or an associated regulatory region. HDR, on the other hand, is a preferred pathway for producing targeted knock-ins, knockouts, or insertions of specific mutations in the presence of a repair template with homologous sequences. Several methods are known to a skilled artisan to improve HDR efficiency, including, for example, chemical modulation (e.g., treating cells with inhibitors of key enzymes in the NHEJ pathway); timed delivery of the gene editing system at S and G2 phases of the cell cycle; cell cycle arrest at S and G2 phases; and introduction of repair templates with homology sequences. The methods provided herein may utilize HDR-mediated repair, NHEJ-mediated repair, or a combination thereof.

In some embodiments, the methods provided herein for HDR-mediated insertion utilize a site-directed nuclease, including, for example, zinc finger nucleases (ZFNs), transcription activator-like effector nucleases (TALENs), meganucleases, transposases, and clustered regularly interspaced short palindromic repeat (CRISPR)/Cas systems.

a. ZFNs

ZFNs are fusion proteins comprising an array of site-specific DNA binding domains adapted from zinc finger-containing transcription factors attached to the endonuclease domain of the bacterial Fokl restriction enzyme. A ZFN may have one or more (e.g., 1, 2, 3, 4, 5, 6, 7, 8, 9, 10, or more) of the DNA binding domains or zinc finger domains. See, e.g., Carroll et al., Genetics Society of America (2011) 188:773-782; Kim et al., Proc. Natl. Acad. Sci. USA (1996) 93:1156-1160. Each zinc finger domain is a small protein structural motif stabilized by one or more zinc ions and usually recognizes a 3- to 4-bp DNA sequence. Tandem domains can thus potentially bind to an extended nucleotide sequence that is unique within a cell's genome.

Various zinc fingers of known specificity can be combined to produce multi-finger polypeptides which recognize about 6, 9, 12, 15, or 18-bp sequences. Various selection and modular assembly techniques are available to generate zinc fingers (and combinations thereof) recognizing specific sequences, including phage display, yeast one-hybrid systems, bacterial one-hybrid and two-hybrid systems, and mammalian cells. Zinc fingers can be engineered to bind a predetermined nucleic acid sequence. Criteria to engineer a zinc finger to bind to a predetermined nucleic acid sequence are known in the art. See, e.g., Sera et al., Biochemistry (2002) 41:7074-7081; Liu et al., Bioinformatics (2008) 24:1850-1857.

ZFNs containing Fokl nuclease domains or other dimeric nuclease domains function as a dimer. Thus, a pair of ZFNs are required to target non-palindromic DNA sites. The two individual ZFNs must bind opposite strands of the DNA with their nucleases properly spaced apart. See Bitinaite et al., Proc. Natl. Acad. Sci. USA (1998) 95:10570-10575. To cleave a specific site in the genome, a pair of ZFNs are designed to recognize two sequences flanking the site, one on the forward strand and the other on the reverse strand. Upon binding of the ZFNs on either side of the site, the nuclease domains dimerize and cleave the DNA at the site, generating a DSB with 5′ overhangs. HDR can then be utilized to introduce a specific mutation, with the help of a repair template containing the desired mutation flanked by homology arms. The repair template is usually an exogenous double-stranded DNA vector introduced to the cell. See Miller et al., Nat. Biotechnol. (2011) 29:143-148; Hockemeyer et al., Nat. Biotechnol. (2011) 29:731-734.

b. TALENs

TALENs are another example of an artificial nuclease which can be used to edit a target gene. TALENs are derived from DNA binding domains termed TALE repeats, which usually comprise tandem arrays with 10 to 30 repeats that bind and recognize extended DNA sequences. Each repeat is 33 to 35 amino acids in length, with two adjacent amino acids (termed the repeat-variable di-residue, or RVD) conferring specificity for one of the four DNA base pairs. Thus, there is a one-to-one correspondence between the repeats and the base pairs in the target DNA sequences.

TALENs are produced artificially by fusing one or more TALE DNA binding domains (e.g., 1, 2, 3, 4, 5, 6, 7, 8, 9, 10 or more) to a nuclease domain, for example, a Fokl endonuclease domain. See Zhang, Nature Biotech. (2011) 29:149-153. Several mutations to Fokl have been made for its use in TALENs; these, for example, improve cleavage specificity or activity. See Cermak et al., Nucl. Acids Res. (2011) 39:e82; Miller et al., Nature Biotech. (2011) 29:143-148; Hockemeyer et al., Nature Biotech. (2011) 29:731-734; Wood et al., Science (2011) 333:307; Doyon et al., Nature Methods (2010) 8:74-79; Szczepek et al., Nature Biotech (2007) 25:786-793; Guo et al., J. Mol. Biol. (2010) 200:96. The Fokl domain functions as a dimer, requiring two constructs with unique DNA binding domains for sites in the target genome with proper orientation and spacing. Both the number of amino acid residues between the TALE DNA binding domain and the Fokl nuclease domain and the number of bases between the two individual TALEN binding sites appear to be important parameters for achieving high levels of activity. Miller et al., Nature Biotech. (2011) 29:143-148.

By combining engineered TALE repeats with a nuclease domain, a site-specific nuclease can be produced specific to any desired DNA sequence. Similar to ZFNs, TALENs can be introduced into a cell to generate DSBs at a desired target site in the genome, and so can be used to knock out genes or knock in mutations in similar, HDR-mediated pathways. See Boch, Nature Biotech. (2011) 29:135-136; Boch et al., Science (2009) 326:1509-1512; Moscou et al., Science (2009) 326:3501.

c. Meganucleases

Meganucleases are enzymes in the endonuclease family which are characterized by their capacity to recognize and cut large DNA sequences (from 14 to 40 base pairs). Meganucleases are grouped into families based on their structural motifs which affect nuclease activity and/or DNA recognition. The most widespread and best known meganucleases are the proteins in the LAGLIDADG family, which owe their name to a conserved amino acid sequence. See Chevalier et al., Nucleic Acids Res. (2001) 29(18): 3757-3774. On the other hand, the GIY-YIG family members have a GIY-YIG module, which is 70-100 residues long and includes four or five conserved sequence motifs with four invariant residues, two of which are required for activity. See Van Roey et al., Nature Struct. Biol. (2002) 9:806-811. The His-Cys family meganucleases are characterized by a highly conserved series of histidines and cysteines over a region encompassing several hundred amino acid residues. See Chevalier et al., Nucleic Acids Res. (2001) 29(18):3757-3774. Members of the NHN family are defined by motifs containing two pairs of conserved histidines surrounded by asparagine residues. See Chevalier et al., Nucleic Acids Res. (2001) 29(18):3757-3774.

Because the chance of identifying a natural meganuclease for a particular target DNA sequence is low due to the high specificity requirement, various methods including mutagenesis and high throughput screening methods have been used to create meganuclease variants that recognize unique sequences. Strategies for engineering a meganuclease with altered DNA-binding specificity, e.g., to bind to a predetermined nucleic acid sequence are known in the art. See, e.g., Chevalier et al., Mol. Cell. (2002) 10:895-905; Epinat et al., Nucleic Acids Res (2003) 31:2952-2962; Silva et al., J Mol. Biol. (2006) 361:744-754; Seligman et al., Nucleic Acids Res (2002) 30:3870-3879; Sussman et al., J Mol Biol (2004) 342:31-41; Doyon et al., J Am Chem Soc (2006) 128:2477-2484; Chen et al., Protein Eng Des Sel (2009) 22:249-256; Arnould et al., J Mol Biol. (2006) 355:443-458; Smith et al., Nucleic Acids Res. (2006) 363(2):283-294.

Like ZFNs and TALENs, Meganucleases can create DSBs in the genomic DNA, which can create a frame-shift mutation if improperly repaired, e.g., via NHEJ, leading to a decrease in the expression of a target gene in a cell. Alternatively, foreign DNA can be introduced into the cell along with the meganuclease. Depending on the sequences of the foreign DNA and chromosomal sequence, this process can be used to modify the target gene. See Silva et al., Current Gene Therapy (2011) 11:11-27.

d. Transposases

Transposases are enzymes that bind to the end of a transposon and catalyze its movement to another part of the genome by a cut and paste mechanism or a replicative transposition mechanism. By linking transposases to other systems such as the CRISPR/Cas system, new gene editing tools can be developed to enable site specific insertions or manipulations of the genomic DNA. There are two known DNA integration methods using transposons which use a catalytically inactive Cas effector protein and Tn7-like transposons. The transposase-dependent DNA integration does not provoke DSBs in the genome, which may guarantee safer and more specific DNA integration.

e. CRISPR/Cas

The CRISPR system was originally discovered in prokaryotic organisms (e.g., bacteria and archaea) as a system involved in defense against invading phages and plasmids that provides a form of acquired immunity. Now it has been adapted and used as a popular gene editing tool in research and clinical applications.

CRISPR/Cas systems generally comprise at least two components: one or more guide RNAs (gRNAs) and a Cas protein. The Cas protein is a nuclease that introduces a DSB into the target site. CRISPR-Cas systems fall into two major classes: class 1 systems use a complex of multiple Cas proteins to degrade nucleic acids; class 2 systems use a single large Cas protein for the same purpose. Class 1 is divided into types I, III, and IV; class 2 is divided into types II, V, and VI. Different Cas proteins adapted for gene editing applications include, but are not limited to, Cas3, Cas4, Cas5, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr5, Cse1, Cse2, Csf1, Csm2, Csn2, Csx10, Csx11, Csy1, Csy2, Csy3, and MAD7. See, e.g., Jinek et al., Science (2012) 337 (6096):816-821; Dang et al., Genome Biology (2015) 16:280; Ran et al., Nature (2015) 520:186-191; Zetsche et al., Cell (2015) 163:759-771; Strecker et al., Nature Comm. (2019) 10:212; Yan et al., Science (2019) 363:88-91. The most widely used Cas9 is a type II Cas protein and is disclosed herein as illustrative. These Cas proteins may be originated from different source species. For example, Cas9 can be derived from S. pyogenes or S. aureus.

In the original microbial genome, the type II CRISPR system incorporates sequences from invading DNA between CRISPR repeat sequences encoded as arrays within the host genome. Transcripts from the CRISPR repeat arrays are processed into CRISPR RNAs (crRNAs) each harboring a variable sequence transcribed from the invading DNA, known as the “protospacer” sequence, as well as part of the CRISPR repeat. Each crRNA hybridizes with a second transactivating CRISPR RNA (tracrRNA), and these two RNAs form a complex with the Cas9 nuclease. The protospacer-encoded portion of the crRNA directs the Cas9 complex to cleave complementary target DNA sequences, provided that they are adjacent to short sequences known as “protospacer adjacent motifs” (PAMs).

While the foregoing description has focused on Cas9 nuclease, it should be appreciated that other RNA-guided nucleases exist which utilize gRNAs that differ in some ways from those disclosed to this point. For instance, Cpf1 (CRISPR from Prevotella and Franciscella 1; also known as Cas12a) is an RNA-guided nuclease that only requires a crRNA and does not need a tracrRNA to function.

Since its discovery, the CRISPR system has been adapted for inducing sequence specific DSBs and targeted genome editing in a wide range of cells and organisms spanning from bacteria to eukaryotic cells including human cells. In its use in gene editing applications, artificially designed, synthetic gRNAs have replaced the original crRNA:tracrRNA complexes, including in certain embodiments via a single gRNA. For example, the gRNAs can be single guide RNAs (sgRNAs) composed of a crRNA, a tetraloop, and a tracrRNA. The crRNA usually comprises a complementary region (also called a spacer, usually about 20 nucleotides in length) that is user-designed to recognize a target DNA of interest. The tracrRNA sequence comprises a scaffold region for Cas nuclease binding. The crRNA sequence and the tracrRNA sequence are linked by the tetraloop and each have a short repeat sequence for hybridization with each other, thus generating a chimeric sgRNA. One can change the genomic target of the Cas nuclease by simply changing the spacer or complementary region sequence present in the gRNA. The complementary region will direct the Cas nuclease to the target DNA site through standard RNA-DNA complementary base pairing rules.

In order for the Cas nuclease to function, there must be a PAM immediately downstream of the target sequence in the genomic DNA. Recognition of the PAM by the Cas protein is thought to destabilize the adjacent genomic sequence, allowing interrogation of the sequence by the gRNA and resulting in gRNA-DNA pairing when a matching sequence is present. The specific sequence of PAM varies depending on the species of the Cas gene. For example, the most commonly used Cas9 nuclease derived from S. pyogenes recognizes a PAM sequence of 5′-NGG-3′ or, at less efficient rates, 5′-NAG-3′, where “N” can be any nucleotide. Other Cas nuclease variants with alternative PAMs have also been characterized and successfully used for genome editing, which are summarized in Table 5 below.

TABLE 32
Exemplary Cas nuclease variants and their PAM sequences

| | | PAM Sequence |
|---|---|---|
| CRISPR Nuclease | Source Organism | (5′→3′) |
| SpCas9 | Streptococcus pyogenes | ngg or nag |
| SaCas9 | Staphylococcus aureus | ngrrt or ngrrn |
| NmeCas9 | Neisseria meningitidis | nnnngatt |
| CjCas9 | Campylobacter jejuni | nnnnryac |
| StCas9 | Streptococcus thermophilus | nnagaaw |
| TdCas9 | Treponema denticola | naaaac |
| LbCas12a (Cpf1) | Lachnospiraceae bacterium | tttv |
| AsCas12a (Cpf1) | Acidaminococcus sp. | tttv |
| AacCas12b | Alicyclobacillus acidiphilus | ttn |
| BhCas12b v4 | Bacillus hisashii | attn, tttn, or gttn |
| ErCas12a (MAD7) | Eubacterium rectale | yttn |

r = a or g; y = c or t; w = a or t; v = a or c or g; n = any base

MAD7 recognizes a PAM 5′ to 21 nucleotide spacer sequence. MAD7 associates with a single, small crRNA of 56 nucleotides in total (35 nucleotide scaffold sequence and 21 nucleotide space sequence). Cleavage of DNA by MAD7 results in a staggered cut 19 base pairs and 23 base pairs distal to the PAM. In some embodiments, a MAD7 crRNA comprises one or more chemical modifications known in the art and/or as described herein.

In some embodiments, Cas nucleases may comprise one or more mutations to alter their activity, specificity, recognition, and/or other characteristics. For example, the Cas nuclease may have one or more mutations that alter its fidelity to mitigate off-target effects (e.g., eSpCas9, SpCas9-HF1, HypaSpCas9, HeFSpCas9, and evoSpCas9 high-fidelity variants of SpCas9). For another example, the Cas nuclease may have one or more mutations that alter its PAM specificity.

In some embodiments, CRISPR systems of the present disclosure comprise TnpB polypeptides. In some embodiments, TnpB polypeptides may comprise a Ruv-C-like domain. The RuvC domain may be a split RuvC domain comprising RuvC-I, RuvC-II, and RuvC-Ill subdomains. In some embodiments, a TnpB may further comprise one or more of a HTH domain, a bridge helix domain and a zinc finger domain. TnpB polypeptides do not comprise an HNH domain. In one exemplary embodiment, a TnpB protein comprises, starting at the N-terminus: a HTH domain, a RuvC-I subdomain, a bridge helix domain, a RuvC-II sub-domain, a zinger finger domain, and a RuvC-Ill sub-domain. In some embodiments, a RuvC-Ill sub-domain forms the C-terminus of a TnpB polypeptide. In some embodiments, a TnpB polypeptide is from Epsilonproteobacteria bacterium, Actinoplanes lobatus strain DSM 43150, Actinomadura celluolosilytica strain DSM 45823, Actinomadura namibiensis strain DSM 44197, Alicyclobacillus macrosprangiidus strain DSM 17980, Lipingzhangella halophila strain DSM 102030, or Ktedonobacter recemifer. In some embodiments, a TnpB polypeptide is from Ktedonobacter racemifer, or comprises a conserved RNA region with similarity to the 5′ ITR of K. racemifer TnpB loci. In some embodiments, a TnpB may comprise a Fanzor protein, a TnpB homolog found in eukaryotic genomes. In some embodiments, a CRISPR system comprising a TnpB polypeptide binds a target adjacent motif (TAM) sequence 5′ of a target polynucleotide. In some embodiments, a TAM is a transposon-associated motif. In some embodiments, a TAM sequence comprises TCA. In some embodiments, a TAM sequence comprises TCAC. In some embodiments, a TAM sequence comprises TCAG. In some embodiments, a TAM sequence comprises TCAT. In some embodiments, a TAM sequence comprises TCAA. In some embodiments, a TAM sequence comprises TTCAN. In some embodiments, a TAM sequence comprises TTCAA. In some embodiments, a TAM sequence comprises TTCAG. In some embodiments, a TAM sequence comprises TTGAT.

In certain embodiments, the transgene may function as a DNA repair template to be integrated into the target site through HDR in associated with a gene editing system (e.g., the CRISPR/Cas system) as disclosed herein. Generally, the transgene to be inserted would comprise at least the expression cassette encoding the protein of interest (e.g., the tolerogenic factor) and would optionally also include one or more regulatory elements (e.g., promoters, insulators, enhancers). In certain of these embodiments, the transgene to be inserted would be flanked by homologous sequence immediately upstream and downstream of the target, i.e., left homology arm (LHA) and right homology arm (RHA), specifically designed for the target genomic locus to serve as template for HDR. The length of each homology arm is generally dependent on the size of the insert being introduced, with larger insertions requiring longer homology arms.

In some embodiments, prime editing may be used to engineer exogenous genes, such as exogenous transgenes encoding a tolerogenic factor (e.g., CD47) into specific loci. Prime editing uses an enzyme and a guide RNA. The enzyme is a catalytically impaired Cas9 endonuclease fused to an engineered reverse transcriptase. The guide RNA is a prime editing guide RNA (pegRNA) that includes RNA specified for the target site and encoding the edit, such as insertion of the transgene. See Anzelone et al., Nature (2019) 576:149-157.

In some embodiments, the base editing technology may be used to introduce single-nucleotide variants (SNVs) into DNA or RNA in living cells. Base editing is a CRISPR-Cas9-based genome editing technology that allows the introduction of point mutations in RNAs or DNAs without generating DSBs. Two major classes of base editors have been developed: cytidine base editors (CBEs) allowing C:G to T:A conversions and adenine base editors (ABEs) allowing A:T to G:C conversions. Base editors are composed by a catalytically dead Cas9 (dCas9) or a nickase Cas9 (nCas9) fused to a deaminase and guided by a sgRNA to the locus of interest. The d/nCas9 recognizes a specific PAM sequence and the DNA unwinds thanks to the complementarity between the sgRNA and the DNA sequence usually located upstream of the PAM (also called protospacer). Then, the opposite DNA strand is accessible to the deaminase that converts the bases located in a specific DNA stretch of the protospacer. Compared to HDR-based strategies, base editing is a promising tool to precisely correct genetic mutations as it avoids gene disruption by NHEJ associated with failed HDR-mediated gene correction.

f. Nickases

Nuclease domains of the Cas, in particular the Cas9, nuclease can be mutated independently to generate enzymes referred to as DNA “nickases.” Nickases are capable of introducing a single-strand cut with the same specificity as a regular CRISPR/Cas nuclease system, including for example CRISPR/Cas9. Nickases can be employed to generate double-strand breaks which can find use in gene editing systems (Mali et al., Nat Biotech, 31(9):833-838 (2013); Mali et al. Nature Methods, 10:957-963 (2013); Mali et al., Science, 339(6121):823-826 (2013)). In some instances, when two Cas nickases are used, long overhangs are produced on each of the cleaved ends instead of blunt ends which allows for additional control over precise gene integration and insertion (Mali et al., Nat Biotech, 31(9):833-838 (2013); Mali et al. Nature Methods, 10:957-963 (2013); Mali et al., Science, 339(6121):823-826 (2013)). As both nicking Cas enzymes must effectively nick their target DNA, paired nickases can have lower off-target effects compared to the double-strand-cleaving Cas-based systems (Ran et al., Cell, 155(2):479-480(2013); Mali et al., Nat Biotech, 31(9):833-838 (2013); Mali et al. Nature Methods, 10:957-963 (2013); Mali et al., Science, 339(6121):823-826 (2013)).

### 4. Genomic Loci for Insertion of the Transgene

In some embodiments, the genomic locus for site-directed insertion of one or more polynucleotides (e.g., transgenes, e.g., a transgene encoding one or more tolerogenic factors) is an endogenous B2M gene locus. In some embodiments, the genomic locus for site-directed insertion one or more polynucleotides (e.g., transgenes, e.g., a transgene encoding one or more tolerogenic factors) is an endogenous CIITA gene locus. In some embodiments one or more polynucleotides (e.g., transgenes, e.g., a transgene encoding one or more tolerogenic factors) are inserted into both B2M and CIITA loci. The specific site for insertion within a gene locus may be located within any suitable region of the gene, including but not limited to a gene coding region (also known as a coding sequence or “CDS”), an exon, an intron, a sequence spanning a portion of an exon and a portion of an adjacent intron, or a regulatory region (e.g., promoter, enhancer). In some embodiments, the insertion occurs in one allele of the specific genomic locus. In some embodiments, the insertion occurs in both alleles of the specific genomic locus. In either of these embodiments, the orientation of the transgene inserted into the target genomic locus can be either the same or the reverse of the direction of the endogenous gene in that locus. In some embodiments, two or more transgenes are inserted in the same locus such that the two or more transgenes are carried by a polycistronic vector. Exemplary genomic loci for insertion of a transgene are depicted in Tables 6 and 7.

TABLE 33
Exemplary genomic loci for insertion of exogenous polynucleotides

| | Gene | | Target region |
|---|---|---|---|
| Species | Locus | Ensembl ID | for cleavage |
| human | B2M | ENSG00000166710 | CDS |
| human | CIITA | ENSG00000179583 | CDS |

TABLE 34
Non-limiting examples of Cas9 guide RNAs

| | SEQ | | | | |
|---|---|---|---|---|---|
| | ID | | | Target | |
| Gene | NO: | guide sequence | PAM | site | gRNA cut location |
| B2M | 19 | CGUGAGUAAACCUGAAUCUU | TGG | Exon 2 | chr15: 44, 715, 434 |
| CIITA | 20 | GAUAUUGGCAUAAGCCUCCC | TGG | Exon 3 | chr16: 10, 895, 747 |

5. Guide RNAs (gRNAs) for Site-Directed Insertion

In some embodiments, provided are gRNAs for use in site-directed insertion of a transgene in a B2M and/or CIITA locus according to various embodiments provided herein, especially in association with the CRISPR/Cas system. The gRNAs comprise a crRNA sequence, which in turn comprises a complementary region (also called a spacer) that recognizes and binds a complementary target DNA of interest. The length of the spacer or complementary region is generally between 15 and 30 nucleotides, usually about 20 nucleotides in length, although will vary based on the requirements of the specific CRISPR/Cas system. In certain embodiments, the spacer or complementary region is fully complementary to the target DNA sequence. In other embodiments, the spacer is partially complementary to the target DNA sequence, for example at least 80%, 85%, 90%, 95%, 98%, or 99% complementary.

In certain embodiments, the gRNAs provided herein further comprise a tracrRNA sequence, which comprises a scaffold region for binding to a nuclease. The length and/or sequence of the tracrRNA may vary depending on the specific nuclease being used for editing. In certain embodiments, nuclease binding by the gRNA does not require a tracrRNA sequence. In those embodiments where the gRNA comprises a tracrRNA, the crRNA sequence may further comprise a repeat region for hybridization with complementary sequences of the tracrRNA.

In some embodiments, the gRNAs provided herein comprise two or more gRNA molecules, for example, a crRNA and a tracrRNA, as two separate molecules. In other embodiments, the gRNAs are single guide RNAs (sgRNAs), including sgRNAs comprising a crRNA and a tracrRNA on a single RNA molecule. In certain of these embodiments, the crRNA and tracrRNA are linked by an intervening tetraloop.

In some embodiments, one gRNA can be used in association with a site-directed nuclease for targeted editing of a gene locus of interest. In other embodiments, two or more gRNAs targeting the same gene locus of interest can be used in association with a site-directed nuclease.

In some embodiments, exemplary gRNAs (e.g., sgRNAs) for use with various common Cas nucleases that require both a crRNA and tracrRNA, including Cas9 and Cas12b (C2c1), are provided in Table 35. See, e.g., Jinek et al., Science (2012) 337 (6096):816-821; Dang et al., Genome Biology (2015) 16:280; Ran et al., Nature (2015) 520:186-191; Strecker et al., Nature Comm. (2019) 10:212. For each exemplary gRNA, sequences for different portions of the gRNA, including the complementary region or spacer, crRNA repeat region, tetraloop, and tracrRNA, are shown. In some embodiments, the gRNA comprises all or a portion of the nucleotide sequences set forth in SEQ ID NOs: 21-24. In some embodiments, the gRNA comprises all or a portion of the nucleotide sequences set forth in SEQ ID NOs: 25-28. In some embodiments, the gRNA comprises all or a portion of the nucleotide sequences set forth in SEQ ID NOs: 29-32. In some embodiments, the gRNA comprises all or a portion of the nucleotide sequences set forth in SEQ ID NOs: 33-36.

In some embodiments, the gRNA comprises a crRNA repeat region comprising, consisting of, or consisting essentially of the nucleotide sequence set forth in SEQ ID NO:22, SEQ ID NO:26, SEQ ID NO:30, or SEQ ID NO:35. In some embodiments, the gRNA comprises a tetraloop comprising, consisting of, or consisting essentially of the nucleotide sequence set forth in SEQ ID NO:23 or SEQ ID NO:34. In some embodiments, the gRNA comprises a tracrRNA comprising, consisting of, or consisting essentially of the nucleotide sequence set forth in SEQ ID NO:24, SEQ ID NO:28, SEQ ID NO:32, or SEQ ID NO:33.

TABLE 35
Exemplary gRNA structure and sequence for CRISPR/Cas

| SEQ ID NO: | Sequence (5′→3′) | Description |
|---|---|---|
| 21 | nnnnnnnnnnnnnnnnnnnn | Exemplary spCas9 1 |
| | | Complementary region |
| | | (spacer) |
| 22 | guuuuagagcua | Exemplary spCas9 1 |
| | | crRNA repeat region |
| 23 | gaaa | Exemplary spCas9 1 |
| | | tetraloop |
| 24 | uagcaaguuaaaauaaggcuaguccguuaucaacuugaaaaaguggcaccga | Exemplary spCas9 1 |
| | gucgg | tracrRNA |
| 25 | nnnnnnnnnnnnnnnnnnnn | Exemplary spCas9 2 |
| | | Complementary region |
| | | (spacer) |
| 26 | guuusagagcuaugcug | Exemplary spCas9 2 |
| | | crRNA repeat region |
| 27 | gaaa | Exemplary spCas9 2 |
| | | tetraloop |
| 28 | cagcauagcaaguusaaauaaggcuaguccguuaucaacuugaaaaaguggc | Exemplary spCas9 2 |
| | accgag | tracrRNA |
| 29 | nnnnnnnnnnnnnnnnnnnn | Exemplary saCas9 |
| | | Complementary region |
| | | (spacer) |
| 30 | | Exemplary saCas9 crRNA |
| | | repeat region |
| 31 | gaaa | Exemplary saCas9 |
| | | tetraloop |
| 32 | cagaaucuacuaaaacaaggcaaaaugccguguuuaucucgucaacuuguug | Exemplary saCas9 |
| | gcgaga | tracrRNA |
| 33 | gucgucuauaggacggcgaggacaacgggaagugccaaugugcucuuuccaa | Exemplary AkCas12b |
| | gagcaaacaccccguuggcuucaagaugaccgcucg | tracrRNA |
| 34 | aaaa | Exemplary AkCas12b |
| | | tetraloop |
| 35 | cgagcggucugagaaguggcacu | Exemplary AkCas12b |
| | | crRNA repeat region |
| 36 | nnnnnnnnnnnnnnnnnnnn | Exemplary AkCas12b |
| | | Complementary region |
| | | (spacer) |

s = c or g; n = any base

In some embodiments, the gRNA comprises a complementary region specific to a target gene locus of interest, for example, the B2M locus (e.g., exon 2 of B2M), or the CIITA locus (e.g., exon 3 of CIITA). The complementary region may bind a sequence in any region of the target gene locus, including for example, a CDS, an exon, an intron, a sequence spanning a portion of an exon and a portion of an adjacent intron, or a regulatory region (e.g., promoter, enhancer). Where the target sequence is a CDS, exon, intron, or sequence spanning portions of an exon and intron, the CDS, exon, intron, or exon/intron boundary may be defined according to any splice variant of the target gene. In some embodiments, the genomic locus targeted by the gRNA is located within 4000 bp, within 3500 bp, within 3000 bp, within 2500 bp, within 2000 bp, within 1500 bp, within 1000 bp, or within 500 bp of any of the loci or regions thereof as disclosed herein. Further provided herein are compositions comprising one or more gRNAs provided herein and a Cas protein or a nucleotide sequence encoding a Cas protein. In certain of these embodiments, the one or more gRNAs and a nucleotide sequence encoding a Cas protein are comprised within a vector, for example, a viral vector.

In some embodiments, provided are methods of identifying new loci and/or gRNA sequences for use in the site-directed genomic insertion approaches as disclosed herein. For example, for CRISPR/Cas systems, when an existing gRNA for a particular locus (e.g., within an endogenous B2M or CIITA gene locus) is known, an “inch worming” approach can be used to identify additional loci for targeted insertion of transgenes by scanning the flanking regions on either side of the locus for PAM sequences, which usually occurs about every 100 base pairs (bp) across the genome. The PAM sequence will depend on the particular Cas nuclease used because different nucleases usually have different corresponding PAM sequences. The flanking regions on either side of the locus can be between about 500 to 4000 bp long, for example, about 500 bp, about 1000 bp, about 1500 bp, about 2000 bp, about 2500 bp, about 3000 bp, about 3500 bp, or about 4000 bp long. When a PAM sequence is identified within the search range, a new guide can be designed according to the sequence of that locus for use in site-directed insertion of transgenes. Although the CRISPR/Cas system is disclosed as illustrative, any gene editing approaches as disclosed can be used in this method of identifying new loci, including those using ZFNs, TALENs, meganucleases, and transposases.

In some embodiments, the activity, stability, and/or other characteristics of gRNAs can be altered through the incorporation of chemical and/or sequential modifications. As one example, transiently expressed or delivered nucleic acids can be prone to degradation by, e.g., cellular nucleases. Accordingly, the gRNAs disclosed herein can contain one or more modified nucleosides or nucleotides which introduce stability toward nucleases. While not being bound by a particular theory, it is believed that certain modified gRNAs disclosed herein can exhibit a reduced innate immune response when introduced into a population of cells, particularly the cells of the present technology. As used herein, the term “innate immune response” includes a cellular response to exogenous nucleic acids, including single stranded nucleic acids, generally of viral or bacterial origin, which involves the induction of cytokine expression and release, particularly the interferons, and cell death. Other common chemical modifications of gRNAs to improve stabilities, increase nuclease resistance, and/or reduce immune response include 2′-O-methyl modification, 2′-fluoro modification, 2′-O-methyl phosphorothioate linkage modification, and 2′-O-methyl 3′ thioPACE modification.

One common 3′ end modification is the addition of a poly(A) tract comprising one or more (and typically 5-200) adenine (A) residues. The poly(A) tract can be contained in the nucleic acid sequence encoding the gRNA or can be added to the gRNA during chemical synthesis, or following in vitro transcription using a polyadenosine polymerase (e.g., E. coli poly(A) polymerase). In vivo, poly(A) tracts can be added to sequences transcribed from DNA vectors through the use of polyadenylation signals. Examples of such signals are provided in Tian et al., “Signals for pre-mRNA cleavage and polyadenylation,” Wiley Interdiscip Rev RNA 3(3): 385-396 (2012). Other suitable gRNA modifications include, without limitations, those disclosed in U.S. Patent Application No. US 2017/0073674 A1 and International Publication No. WO 2017/165862 A1, the entire contents of each of which are incorporated by reference herein.

6. Delivery of Gene Editing Systems into a Host Cell

In some embodiments, provided are compositions comprising one or more components of a gene editing system disclosed herein, including one or more gRNAs, a site-directed nuclease (e.g., a Cas nuclease) or a nucleotide sequence encoding a site-directed nuclease protein, and a transgene for targeted insertion. In some embodiments, these compositions are formulated for delivery into a cell.

In some embodiments, components of a gene editing system provided herein, including one or more gRNAs, a site-directed nuclease (e.g., a Cas nuclease) or a nucleotide sequence encoding a site-directed nuclease protein, and a transgene (e.g., a transgene encoding a tolerogenic factor) for targeted insertion, may be delivered into a cell in the form of a delivery vector. The delivery vector can be any type of vector suitable for introduction of nucleotide sequences into a cell, including, for example, plasmids, adenoviral vectors, adeno-associated viral (AAV) vectors such as an AAV6 vector and an AAV9 vector, retroviral vectors, lentiviral vectors, phages, and HDR-based donor vectors. Additional AAV vectors for gene delivery are disclosed in, for example, Wang et al., “Adeno-associated virus vector as a platform for gene therapy deliver,” Nature Reviews Drug Discovery 18: 358-378 (2019), the disclosure is incorporated herein by reference in its entirety. The different components may be introduced into a cell together or separately, and may be delivered in a single vector or multiple vectors.

In some embodiments, the delivery vector may be introduced into a cell by any known method in the field, including, for example, viral transformation, calcium phosphate transfection, lipid-mediated transfection, DEAE-dextran, electroporation, microinjection, nucleoporation, liposomes, nanoparticles, or other methods.

In some embodiments, the present technology provides compositions comprising a delivery vector according to various embodiments disclosed herein. In some embodiments, the compositions may further comprise one or more pharmaceutically acceptable carriers, excipients, preservatives, or a combination thereof. A “pharmaceutically acceptable carrier or excipient” refers to a pharmaceutically acceptable material, composition, or vehicle that is involved in carrying or transporting a compound of interest from one tissue, organ, or portion of the body to another tissue, organ, or portion of the body. For example, the carrier or excipient may be a liquid or solid filler, diluent, excipient, solvent, or encapsulating material, or some combination thereof. Each component of the carrier or excipient must be “pharmaceutically acceptable,” in that it must be compatible with the other ingredients of the formulation. It also must be suitable for contact with any tissue, organ, or portion of the body that it may encounter, meaning that it must not carry a risk of toxicity, irritation, allergic response, immunogenicity, or any other complication that excessively outweighs its therapeutic benefits. Suitable excipients include water, saline, dextrose, glycerol, or the like and combinations thereof. In some embodiments, compositions comprising cells as disclosed herein further comprise a suitable infusion media.

In some embodiments, provided are cells or compositions thereof comprising one or more components of a gene editing system disclosed herein, including one or more gRNAs, a site-directed nuclease (e.g., a Cas nuclease) or a nucleotide sequence encoding a site-directed nuclease protein, and a transgene for targeted insertion.

### K. Expression From Exogenous Polynucleotides

For all of these technologies, well-known recombinant techniques are used, to generate recombinant nucleic acids as outlined herein. In certain embodiments, the recombinant nucleic acids encoding a tolerogenic factor or a chimeric antigen receptor may be operably linked to one or more regulatory nucleotide sequences in an expression construct. Regulatory nucleotide sequences will generally be appropriate for the host cell and recipient subject to be treated. Numerous types of appropriate expression vectors and suitable regulatory sequences are known in the art for a variety of host cells. Typically, the one or more regulatory nucleotide sequences may include, but are not limited to, promoter sequences, leader or signal sequences, ribosomal binding sites, transcriptional start and termination sequences, translational start and termination sequences, and enhancer or activator sequences. Constitutive or inducible promoters as known in the art are also contemplated. The promoters may be either naturally occurring promoters, hybrid promoters that combine elements of more than one promoter, or synthetic promoters. An expression construct may be present in a cell on an episome, such as a plasmid, or the expression construct may be inserted in a chromosome such as in a gene locus. In some embodiment, the expression vector includes a selectable marker gene to allow the selection of transformed host cells. Some embodiments, include an expression vector comprising a nucleotide sequence encoding a variant polypeptide operably linked to at least one regulatory sequence. Regulatory sequence for use herein include promoters, enhancers, and other expression control elements. In some embodiments, an expression vector is designed for the choice of the host cell to be transformed, the particular variant polypeptide desired to be expressed, the vector's copy number, the ability to control that copy number, and/or the expression of any other protein encoded by the vector, such as antibiotic markers.

Examples of suitable mammalian promoters include, for example, promoters from the following genes: elongation factor 1 alpha (EF1α) promoter, CAG promoter, ubiquitin/S27a promoter of the hamster (WO 97/15664), Simian vacuolating virus 40 (SV40) early promoter, adenovirus major late promoter, mouse metallothionein-I promoter, the long terminal repeat region of Rous Sarcoma Virus (RSV), mouse mammary tumor virus promoter (MMTV), Moloney murine leukemia virus Long Terminal repeat region, and the early promoter of human Cytomegalovirus (CMV). Examples of other heterologous mammalian promoters are the actin, immunoglobulin or heat shock promoter(s). In additional embodiments, promoters for use in mammalian host cells can be obtained from the genomes of viruses such as polyoma virus, fowlpox virus (UK 2,211,504 published 5 Jul. 1989), bovine papilloma virus, avian sarcoma virus, cytomegalovirus, a retrovirus, hepatitis-B virus and Simian Virus 40 (SV40). In further embodiments, heterologous mammalian promoters are used. Examples include the actin promoter, an immunoglobulin promoter, and heat-shock promoters. The early and late promoters of SV40 are conveniently obtained as an SV40 restriction fragment which also contains the SV40 viral origin of replication (Fiers et al., Nature 273: 113-120 (1978)). The immediate early promoter of the human cytomegalovirus is conveniently obtained as a HindIII restriction enzyme fragment (Greenaway et al., Gene 18: 355-360 (1982)). The foregoing references are incorporated by reference in their entirety.

In some embodiments, the expression vector is a bicistronic or multicistronic expression vector. Bicistronic or multicistronic expression vectors may include (1) multiple promoters fused to each of the open reading frames; (2) insertion of splicing signals between genes; (3) fusion of genes whose expressions are driven by a single promoter; and (4) insertion of proteolytic cleavage sites between genes (self-cleavage peptide) or insertion of internal ribosomal entry sites (IRESs) between genes.

The process of introducing the polynucleotides described herein into cells can be achieved by any suitable technique. Suitable techniques include calcium phosphate or lipid-mediated transfection, electroporation, fusogens, and transduction or infection using a viral vector. In some embodiments, the polynucleotides are introduced into a cell via viral transduction (e.g., AAV transduction, lentiviral transduction) or otherwise delivered on a viral vector (e.g., fusogen-mediated delivery). In some embodiments, the polynucleotides are introduced into a cell via a fusogen-mediated delivery or a transposase system selected from the group consisting of conditional or inducible transposases, conditional or inducible PiggyBac transposons, conditional or inducible Sleeping Beauty (SB11) transposons, conditional or inducible Mos1 transposons, and conditional or inducible Tol2 transposons.

In some embodiments, the cells provided herein are genetically modified to include one or more exogenous polynucleotides inserted into one or more genomic loci of the hypoimmunogenic cell. In some embodiments, the exogenous polynucleotide encodes a protein of interest, e.g., a chimeric antigen receptor. Any suitable method can be used to insert the exogenous polynucleotide into the genomic locus of the hypoimmunogenic cell including the gene editing methods described herein (e.g., a CRISPR/Cas system). In some embodiments, the exogenous polynucleotide is inserted into at least one allele of the cell using viral transduction, for example, with a vector. In some embodiments, the vector is a pseudotyped, self-inactivating lentiviral vector that carries the exogenous polynucleotide. In some embodiments, the vector is a self-inactivating lentiviral vector pseudotyped with a vesicular stomatitis VSV-G envelope, and which carries the exogenous polynucleotide. In some embodiments, the exogenous polynucleotide is inserted into at least one allele of the cell using viral transduction. In some embodiments, the exogenous polynucleotide is inserted into at least one allele of the cell using a lentivirus based viral vector.

Unlike certain methods of introducing the polynucleotides described herein into cells which generally involve activating cells, such as activating T cells (e.g., CD8′T cells), suitable techniques can be utilized to introduce polynucleotides into non-activated T cells. Suitable techniques include, but are not limited to, activation of T cells, such as CD8′T cells, with one or more antibodies which bind to CD3, CD8, and/or CD28, or fragments or portions thereof (e.g., scFv and VHH) that may or may not be bound to beads. Surprisingly, fusogen-mediated introduction of polynucleotides into T cells is performed in non-activated T cells (e.g., CD8′T cells) that have not been previously contacted with one or more activating antibodies or fragments or portions thereof (e.g., CD3, CD8, and/or CD28). In some embodiments, fusogen-mediated introduction of polynucleotides into T cells is performed in vivo (e.g., after the T cells have been administered to a subject). In other embodiments, fusogen-mediated introduction of polynucleotides into T cells is performed in vitro (e.g., before the T cells are been administered to a subject).

Provided herein are non-activated T cells comprising reduced expression of HLA-A, HLA-B, HLA-C, HLA-DP, HLA-DM, HLA-DOB, HLA-DQ, HLA-DR, B2M, CIITA, TCR-alpha, and/or TCR-beta relative to a wild-type T cell, wherein the non-activated T cell further comprises a first exogenous polynucleotide encoding a chimeric antigen receptor (CAR).

In some embodiments, the non-activated T cell has not been treated with an anti-CD3 antibody, an anti-CD28 antibody, a T cell activating cytokine, or a soluble T cell costimulatory molecule. In some embodiments, the non-activated T cell does not express activation markers. In some embodiments, the non-activated T cell expresses CD3 and CD28, and wherein the CD3 and/or CD28 are inactive.

In some embodiments, the anti-CD3 antibody is OKT3. In some embodiments, the anti-CD28 antibody is CD28.2. In some embodiments, the T cell activating cytokine is selected from the group of T cell activating cytokines consisting of IL-2, IL-7, IL-15, and IL-21. In some embodiments, the soluble T cell costimulatory molecule is selected from the group of soluble T cell costimulatory molecules consisting of an anti-CD28 antibody, an anti-CD80 antibody, an anti-CD86 antibody, an anti-CD137L antibody, and an anti-ICOS-L antibody.

In some embodiments, the non-activated T cell is a primary T cell. In other embodiments, the non-activated T cell is differentiated from the engineered CAR-T cells of the present disclosure. In some embodiments, the T cell is a CD8′T cell.

In some embodiments, the first exogenous polynucleotide encodes CD22-specific CAR.

In some embodiments, the first and/or second exogenous polynucleotide is carried by a viral vector, including a lentiviral vector. In some embodiments, the first and/or second exogenous polynucleotide is carried by a lentiviral vector that comprises a CD8 binding agent. In some embodiments, the first and/or second exogenous polynucleotide is introduced into the cells using fusogen-mediated delivery or a transposase system selected from the group consisting of conditional or inducible transposases, conditional or inducible PiggyBac transposons, conditional or inducible Sleeping Beauty (SB11) transposons, conditional or inducible Mos1 transposons, and conditional or inducible Tol2 transposons.

In some embodiments, the non-activated T cell further comprises a second exogenous polynucleotide encoding CD47. In some embodiments, the first and/or second exogenous polynucleotides are inserted into a specific locus of at least one allele of the T cell. In some embodiments, the specific locus is selected from the group consisting of a safe harbor or target locus, a target locus, a B2M locus, a CIITA locus, a TRAC locus, and a TRB locus. In some embodiments, the second exogenous polynucleotide encoding CD47 is inserted into the specific locus selected from the group consisting of a safe harbor or target locus, a target locus, a B2M locus, a CIITA locus, a TRAC locus and a TRB locus. In some embodiments, the first exogenous polynucleotide encoding the CAR is inserted into the specific locus selected from the group consisting of a safe harbor or target locus, a target locus, a B2M locus, a CIITA locus, a TRAC locus and a TRB locus. In some embodiments, the second exogenous polynucleotide encoding CD47 and the first exogenous polynucleotide encoding the CAR are inserted into different loci. In some embodiments, the second exogenous polynucleotide encoding CD47 and the first exogenous polynucleotide encoding the CAR are inserted into the same locus. In some embodiments, the second exogenous polynucleotide encoding CD47 and the first exogenous polynucleotide encoding the CAR are inserted into the B2M locus. In some embodiments, the second exogenous polynucleotide encoding CD47 and the first exogenous polynucleotide encoding the CAR are inserted into the CIITA locus. In some embodiments, the second exogenous polynucleotide encoding CD47 and the first exogenous polynucleotide encoding the CAR are inserted into the TRAC locus. In some embodiments, the second exogenous polynucleotide encoding CD47 and the first exogenous polynucleotide encoding the CAR are inserted into the TRB locus. In some embodiments, the second exogenous polynucleotide encoding CD47 and the first exogenous polynucleotide encoding the CAR are inserted into the safe harbor or target locus. In some embodiments, the safe harbor or target locus is selected from the group consisting of a CCR5 gene locus, a CXCR4 gene locus, a PPP1R12C gene locus, an albumin gene locus, a SHS231 gene locus, a CLYBL gene locus, a Rosa gene locus, an F3 (CD142) gene locus, a MICA gene locus, a MICB gene locus, a LRP1 (CD91) gene locus, a HMGB1 gene locus, an ABO gene locus, an RHD gene locus, a FUT1 locus, and a KDM5D gene locus.

In some embodiments, the non-activated T cell does not express HLA-A, HLA-B, and/or HLA-C antigens. In some embodiments, the non-activated T cell does not express B2M. In some embodiments, the non-activated T cell does not express HLA-DP, HLA-DM, HLA-DOB, HLA-DQ, and/or HLA-DR antigens. In some embodiments, the non-activated T cell does not express CIITA. In some embodiments, the non-activated T cell does not express TCR-alpha. In some embodiments, the non-activated T cell does not express TCR-beta. In some embodiments, the non-activated T cell does not express TCR-alpha and TCR-beta.

In some embodiments, the non-activated T cell is a B2M^{indel/indel}, CIITA^{indel/indel}, TRAC^{indel/indel} cell comprising second exogenous polynucleotide encoding CD47 and/or the first exogenous polynucleotide encoding CAR inserted into the TRAC locus. In some embodiments, the non-activated T cell is a B2M^{indel/indel}, CIITA^{indel/indel}, TRAC^{indel/indel} cell comprising the second exogenous polynucleotide encoding CD47 and the first exogenous polynucleotide encoding CAR inserted into the TRAC locus. In some embodiments, the non-activated T cell is a B2M^{indel/indel}, CIITA^{indel/indel}, TRAC^{indel/indel} cell comprising second exogenous polynucleotide encoding CD47 and/or the first exogenous polynucleotide encoding CAR inserted into the TRB locus. In some embodiments, the non-activated T cell is a B2M^{indel/indel}, CIITA^{indel/indel}, TRAC^{indel/indel} cell comprising the second exogenous polynucleotide encoding CD47 and the first exogenous polynucleotide encoding CAR inserted into the TRB locus. In some embodiments, the non-activated T cell is a B2M^{indel/indel}, CIITA^{indel/indel}, TRAC^{indel/indel} cell comprising second exogenous polynucleotide encoding CD47 and/or the first exogenous polynucleotide encoding CAR inserted into the B2M locus. In some embodiments, the non-activated T cell is a B2M^{indel/indel}, CIITA^{indel/indel}, TRAC^{indel/indel} cell comprising the second exogenous polynucleotide encoding CD47 and the first exogenous polynucleotide encoding CAR inserted into a B2M locus. In some embodiments, the non-activated T cell is a B2M^{indel/indel}, CIITA^{indel/indel}, TRAC^{indel/indel} cell comprising second exogenous polynucleotide encoding CD47 and/or the first exogenous polynucleotide encoding CAR inserted into the CIITA locus. In some embodiments, the non-activated T cell is a B2M^{indel/indel}, CIITA^{indel/indel}, TRAC^{indel/indel} cell comprising the second exogenous polynucleotide encoding CD47 and the first exogenous polynucleotide encoding CAR inserted into a CIITA locus.

In some embodiments, the non-activated T cell is a B2M^{indel/indel}, CIITA^{indel/indel}, TRB^{indel/indel} cell comprising second exogenous polynucleotide encoding CD47 and/or the first exogenous polynucleotide encoding CAR inserted into the TRAC locus. In some embodiments, the non-activated T cell is a B2M^{indel/indel}, CIITA^{indel/indel}, TRB^{indel/indel} cell comprising the second exogenous polynucleotide encoding CD47 and the first exogenous polynucleotide encoding CAR inserted into the TRAC locus. In some embodiments, the non-activated T cell is a B2M^{indel/indel}, CIITA^{indel/indel}, TRB^{indel/indel} cell comprising second exogenous polynucleotide encoding CD47 and/or the first exogenous polynucleotide encoding CAR inserted into the TRB locus. In some embodiments, the non-activated T cell is a B2M^{indel/indel}, CIITA^{indel/indel}, TRB^{indel/indel} cell comprising the second exogenous polynucleotide encoding CD47 and the first exogenous polynucleotide encoding CAR inserted into the TRB locus. In some embodiments, the non-activated T cell is a B2M^{indel/indel}, CIITA^{indel/indel}, TRB^{indel/indel} cell comprising second exogenous polynucleotide encoding CD47 and/or the first exogenous polynucleotide encoding CAR inserted into the B2M locus. In some embodiments, the non-activated T cell is a B2M^{indel/indel}, CIITA^{indel/indel}, TRB^{indel/indel} cell comprising the second exogenous polynucleotide encoding CD47 and the first exogenous polynucleotide encoding CAR inserted into a B2M locus. In some embodiments, the non-activated T cell is a B2M^{indel/indel}, CIITA^{indel/indel}, TRB^{indel/indel} cell comprising second exogenous polynucleotide encoding CD47 and/or the first exogenous polynucleotide encoding CAR inserted into the CIITA locus. In some embodiments, the non-activated T cell is a B2M^{indel/indel}, CIITA^{indel/indel}, TRB^{indel/indel} cell comprising the second exogenous polynucleotide encoding CD47 and the first exogenous polynucleotide encoding CAR inserted into a CIITA locus.

Provided herein are engineered CAR-T cells comprising reduced expression of HLA-A, HLA-B, HLA-C, HLA-DP, HLA-DM, HLA-DOB, HLA-DQ, HLA-DR, B2M, CIITA, TCR-alpha, and/or TCR-beta relative to a wild-type T cell, wherein the engineered CAR-T cell further comprises a first exogenous polynucleotide encoding a chimeric antigen receptor (CAR) carried by a viral vector, including a lentiviral vector. Provided herein are engineered CAR-T cells comprising reduced expression of HLA-A, HLA-B, HLA-C, HLA-DP, HLA-DM, HLA-DOB, HLA-DQ, HLA-DR, B2M, CIITA, TCR-alpha, and/or TCR-beta relative to a wild-type T cell, wherein the engineered CAR-T cell further comprises a first exogenous polynucleotide encoding a CAR carried by a lentiviral vector that comprises a CD8 binding agent.

In some embodiments, the engineered CAR-T cell is a primary T cell. In other embodiments, the engineered CAR-T cell is differentiated from the hypoimmunogenic cell of the present disclosure. In some embodiments, the T cell is a CD8⁺ T cell. In some embodiments, the T cell is a CD4′T cell.

In some embodiments, the engineered CAR-T cell does not express activation markers. In some embodiments, the engineered CAR-T cell expresses CD3 and CD28, and wherein the CD3 and/or CD28 are inactive.

In some embodiments, the engineered CAR-T cell has not been treated with an anti-CD3 antibody, an anti-CD28 antibody, a T cell activating cytokine, or a soluble T cell costimulatory molecule. In some embodiments, the anti-CD3 antibody is OKT3, wherein the anti-CD28 antibody is CD28.2, wherein the T cell activating cytokine is selected from the group of T cell activating cytokines consisting of IL-2, IL-7, IL-15, and IL-21, and wherein soluble T cell costimulatory molecule is selected from the group of soluble T cell costimulatory molecules consisting of an anti-CD28 antibody, an anti-CD80 antibody, an anti-CD86 antibody, an anti-CD137L antibody, and an anti-ICOS-L antibody. In some embodiments, the engineered CAR-T cell has not been treated with one or more T cell activating cytokines selected from the group consisting of IL-2, IL-7, IL-15, and IL-21. In some instances, the cytokine is IL-2. In some embodiments, the one or more cytokines is IL-2 and another selected from the group consisting of IL-7, IL-15, and IL-21.

In some embodiments, the engineered CAR-T cell further comprises a second exogenous polynucleotide encoding CD47. In some embodiments, the first and/or second exogenous polynucleotides are inserted into a specific locus of at least one allele of the T cell. In some embodiments, the specific locus is selected from the group consisting of a safe harbor or target locus, a target locus, a B2M locus, a CIITA locus, a TRAC locus, and a TRB locus. In some embodiments, the second exogenous polynucleotide encoding CD47 is inserted into the specific locus selected from the group consisting of a safe harbor or target locus, a target locus, a B2M locus, a CIITA locus, a TRAC locus and a TRB locus. In some embodiments, the first exogenous polynucleotide encoding the CAR is inserted into the specific locus selected from the group consisting of a safe harbor or target locus, a target locus, a B2M locus, a CIITA locus, a TRAC locus and a TRB locus. In some embodiments, the second exogenous polynucleotide encoding CD47 and the first exogenous polynucleotide encoding the CAR are inserted into different loci. In some embodiments, the second exogenous polynucleotide encoding CD47 and the first exogenous polynucleotide encoding the CAR are inserted into the same locus. In some embodiments, the second exogenous polynucleotide encoding CD47 and the first exogenous polynucleotide encoding the CAR are inserted into the B2M locus, the CIITA locus, the TRAC locus, the TRB locus, or the safe harbor or target locus. In some embodiments, the safe harbor or target locus is selected from the group consisting of a CCR5 gene locus, a CXCR4 gene locus, a PPP1R12C gene locus, an albumin gene locus, a SHS231 gene locus, a CLYBL gene locus, a Rosa gene locus, an F3 (CD142) gene locus, a MICA gene locus, a MICB gene locus, a LRP1 (CD91) gene locus, a HMGB1 gene locus, an ABO gene locus, an RHD gene locus, a FUT1 locus, and a KDM5D gene locus.

In some embodiments, the CAR is selected from the group consisting of a CD19-specific CAR and a CD22-specific CAR. In some embodiments, the CAR is a CD19-specific CAR. In some embodiments, the CAR is a CD22-specific CAR. In some embodiments, the CAR comprises an antigen binding domain that binds to any one selected from the group consisting of CD19, CD22, CD38, CD123, CD138, BCMA, GPRC5D, CD70, and CD79b.

In some embodiments, the engineered CAR-T cell does not express HLA-A, HLA-B, and/or HLA-C antigens, wherein the engineered CAR-T cell does not express B2M, wherein the engineered CAR-T cell does not express HLA-DP, HLA-DM, HLA-DOB, HLA-DQ, and/or HLA-DR antigens, wherein the engineered CAR-T cell does not express CIITA, and/or wherein the engineered CAR-T cell does not express TCR-alpha and TCR-beta.

In some embodiments, the engineered CAR-T cell is a B2M^{indel/indel}, CIITA^{indel/indel}, TRAC^{indel/indel} cell comprising the second exogenous polynucleotide encoding CD47 and/or the first exogenous polynucleotide encoding CAR inserted into the TRAC locus, into the TRB locus, into the B2M locus, or into the CIITA locus. In some embodiments, the engineered CAR-T cell is a B2M^{indel/indel}, CIITA^{indel/indel}, TRB^{indel/indel} cell comprising the second exogenous polynucleotide encoding CD47 and/or the first exogenous polynucleotide encoding CAR inserted into the TRAC locus, into the TRB locus, into the B2M locus, or into the CIITA locus.

In some embodiments, the non-activated T cell and/or the engineered CAR-T cell of the present disclosure are in a subject. In other embodiments, the non-activated T cell and/or the engineered CAR-T cell of the present disclosure are in vitro.

In some embodiments, the non-activated T cell and/or the engineered CAR-T cell of the present disclosure express a CD8 binding agent. In some embodiments, the CD8 binding agent is an anti-CD8 antibody. In some embodiments, the anti-CD8 antibody is selected from the group consisting of a mouse anti-CD8 antibody, a rabbit anti-CD8 antibody, a human anti-CD8 antibody, a humanized anti-CD8 antibody, a camelid (e.g., llama, alpaca, camel) anti-CD8 antibody, and a fragment thereof. In some embodiments, the fragment thereof is an scFv or a VHH. In some embodiments, the CD8 binding agent binds to a CD8 alpha chain and/or a CD8 beta chain.

In some embodiments, the CD8 binding agent is fused to a transmembrane domain incorporated in the viral envelope. In some embodiments, the lentivirus vector is pseudotyped with a viral fusion protein. In some embodiments, the viral fusion protein comprises one or more modifications to reduce binding to its native receptor.

In some embodiments, the viral fusion protein is fused to the CD8 binding agent. In some embodiments, the viral fusion protein comprises Nipah virus F glycoprotein and Nipah virus G glycoprotein fused to the CD8 binding agent. In some embodiments, the lentivirus vector does not comprise a T cell activating molecule or a T cell costimulatory molecule. In some embodiments, the lentivirus vector encodes the first exogenous polynucleotide and/or the second exogenous polynucleotide.

In some embodiments, following transfer into a first subject, the non-activated T cell or the engineered CAR-T cell exhibits one or more responses selected from the group consisting of (a) a T cell response, (b) an NK cell response, and (c) a macrophage response, that are reduced as compared to a wild-type cell following transfer into a second subject. In some embodiments, the first subject and the second subject are different subjects. In some embodiments, the macrophage response is engulfment.

In some embodiments, following transfer into a subject, the non-activated T cell or the engineered CAR-T cell exhibits one or more selected from the group consisting of (a) reduced TH1 activation in the subject, (b) reduced NK cell killing in the subject, and (c) reduced killing by whole PBMCs in the subject, as compared to a wild-type cell following transfer into the subject.

In some embodiments, following transfer into a subject, the non-activated T cell or the engineered CAR-T cell elicits one or more selected from the group consisting of (a) reduced donor specific antibodies in the subject, (b) reduced IgM or IgG antibodies in the subject, and (c) reduced complement-dependent cytotoxicity (CDC) in a subject, as compared to a wild-type cell following transfer into the subject.

In some embodiments, the non-activated T cell or the engineered CAR-T cell is transduced with a lentivirus vector comprising a CD8 binding agent within the subject. In some embodiments, the lentivirus vector carries a gene encoding the CAR and/or CD47.

In some embodiments, the gene encoding the CAR and/or CD47 is introduced into the cells using fusogen-mediated delivery, a transposase system selected from the group consisting of transposases, PiggyBac transposons, Sleeping Beauty (SB11) transposons, Mos1 transposons, and Tol2 transposons, or a viral vector, including a lentiviral vector.

Provided herein are pharmaceutical compositions comprising a population of the non-activated T cells and/or the engineered CAR-T cells of the present disclosure and a pharmaceutically acceptable additive, carrier, diluent or excipient.

Provided herein are methods comprising administering to a subject a composition comprising a population of the non-activated T cells and/or the engineered CAR-T cells of the present disclosure, or one or more the pharmaceutical compositions of the present disclosure.

In some embodiments, the subject is not administered a T cell activating treatment before, after, and/or concurrently with administration of the composition. In some embodiments, the T cell activating treatment comprises lymphodepletion.

Provided herein are methods of treating a subject suffering from cancer, comprising administering to a subject a composition comprising a population of the non-activated T cells and/or the engineered CAR-T cells of the present disclosure, or one or more the pharmaceutical compositions of the present disclosure, wherein the subject is not administered a T cell activating treatment before, after, and/or concurrently with administration of the composition. In some embodiments, the T cell activating treatment comprises lymphodepletion.

Provided herein are methods for expanding T cells capable of recognizing and killing tumor cells in a subject in need thereof within the subject, comprising administering to a subject a composition comprising a population of the non-activated T cells and/or the engineered CAR-T cells of the present disclosure, or one or more the pharmaceutical compositions of the present disclosure, wherein the subject is not administered a T cell activating treatment before, after, and/or concurrently with administration of the composition. In some embodiments, the T cell activating treatment comprises lymphodepletion.

Provided herein are dosage regimens for treating a condition, disease or disorder in a subject comprising administration of a pharmaceutical composition comprising a population of the non-activated T cells and/or the engineered CAR-T cells of the present disclosure, or one or more the pharmaceutical compositions of the present disclosure, and a pharmaceutically acceptable additive, carrier, diluent or excipient, wherein the pharmaceutical composition is administered in about 1-3 therapeutically effective doses. Provided herein are dosage regimens for treating a condition, disease or disorder in a subject comprising administration of a pharmaceutical composition comprising a population of the non-activated T cells and/or the engineered CAR-T cells of the present disclosure, or one or more the pharmaceutical compositions of the present disclosure, and a pharmaceutically acceptable additive, carrier, diluent or excipient, wherein the pharmaceutical composition is administered in about 1-3 clinically effective doses.

Once altered, the presence of expression of any of the molecule described herein can be assayed using known techniques, such as Western blots, ELISA assays, FACS assays, other immunoassays, RT-PCR, and the like.

### L. Exogenous Polynucleotides

In some embodiments, the engineered CAR-T cells provided herein are genetically modified to include one or more exogenous polynucleotides inserted into one or more genomic loci of the hypoimmunogenic cell. In some embodiments, the exogenous polynucleotide encodes a protein of interest, e.g., a chimeric antigen receptor. Any suitable method can be used to insert the exogenous polynucleotide into the genomic locus of the hypoimmunogenic cell including the gene editing methods described herein (e.g., a CRISPR/Cas system). In some embodiments, the one or more exogenous polynucleotides are inserted into at least one allele of the cell using viral transduction, for example, with a vector. In some embodiments, the vector is a pseudotyped, self-inactivating lentiviral vector that carries the one or more exogenous polynucleotides. In some embodiments, the vector is a self-inactivating lentiviral vector pseudotyped with a vesicular stomatitis VSV-G envelope, and which carries the one or more exogenous polynucleotides. In some embodiments, the one or more exogenous polynucleotides are inserted into at least one allele of the cell using viral transduction. In some embodiments, the one or more exogenous polynucleotide are inserted into at least one allele of the cell using a lentivirus based viral vector.

The exogenous polynucleotide can be inserted into any suitable genomic loci of the hypoimmunogenic cell. In some embodiments, the exogenous polynucleotide is inserted into a safe harbor or target locus as described herein. Suitable safe harbor and target loci include, but are not limited to, a CCR5 gene, a CXCR4 gene, a PPP1R12C (also known as AAVS1) gene, an albumin gene, a SHS231 locus, a CLYBL gene, a Rosa gene (e.g., ROSA26), an F3 gene (also known as CD142), a MICA gene, a MICB gene, a LRP1 gene (also known as CD91), a HMGB1 gene, an ABO gene, a RHD gene, a FUT1 gene, a PDGFRa gene, an OLIG2 gene, a GFAP gene, and a KDM5D gene (also known as HY). In some embodiments, the exogenous polynucleotide is interested into an intron, exon, or coding sequence region of the safe harbor or target gene locus. In some embodiments, the exogenous polynucleotide is inserted into an endogenous gene wherein the insertion causes silencing or reduced expression of the endogenous gene. In some embodiments, the polynucleotide is inserted in a B2M, CIITA, TRAC, TRB, PD-1 or CTLA-4 gene locus. Exemplary genomic loci for insertion of an exogenous polynucleotide are depicted in Table 36.

TABLE 36
Exemplary genomic loci for insertion of exogenous polynucleotides

| | | | | Target region | |
|---|---|---|---|---|---|
| Number | species | Name | Ensembl ID | for cleavage | Also known as |
| 1 | human | B2M | ENSG00000166710 | CDS | |
| 2 | human | CIITA | ENSG00000179583 | CDS | |
| 3 | human | TRAC | ENSG00000277734 | CDS | |
| 4 | human | PPP1R12C | ENSG00000125503 | Intron 1 and 2 | AAVS1 |
| 5 | human | CLYBL | ENSG00000125246 | Intron 2 | |
| 6 | human | CCR5 | ENSG00000160791 | Exons 1-3, | |
| | | | | introns 1-2, and | |
| | | | | CDS | |
| 7 | human | THUMPD3-AS1 | ENSG00000206573 | Intron 1 | ROSA26 |
| 8 | human | Ch- 4: 58,976,613 | | 500 bp window | SHS231 |
| 9 | human | F3 | ENSG00000117525 | CDS | CD142 |
| 10 | human | MICA | ENSG00000204520 | CDS | |
| 11 | human | MICB | ENSG00000204516 | CDS | |
| 12 | human | LRP1 | ENSG00000123384 | CDS | |
| 13 | human | HMGB1 | ENSG00000189403 | CDS | |
| 14 | human | ABO | ENSG00000175164 | CDS | |
| 15 | human | RHD | ENSG00000187010 | CDS | |
| 16 | human | FUT1 | ENSG00000174951 | CDS | |
| 17 | human | KDM5D | ENSG00000012817 | CDS | HY |
| 18 | human | CD52 | ENSG00000169442 | CDS | |
| 19 | human | CD70 | ENSG00000125726 | CDS | |
| 20 | human | CD155 | ENSG00000073008 | CDS | PVR/JAM-A |

TABLE 37
Non-limiting examples of Cas9 guide RNAs

| | SEQ | | | | |
|---|---|---|---|---|---|
| | ID | | | Target | |
| Gene | NO: | guide sequence | PAM | site | gRNA cut location |
| ABO | 1 | UCUCUCCAUGUGCAGUAGGA | AGG | Exon 7 | chr9: 133, 257, 541 |
| FUT1 | 2 | CUGGAUGUCGGAGGAGUACG | CGG | Exon 4 | chr19: 48, 750, 822 |
| RH | 3 | GUCUCCGGAAACUCGAGGUG | AGG | Exon 2 | chr1: 25, 284, 622 |
| F3 (CD142) | 4 | ACAGUGUAGACUUGAUUGAC | GGG | Exon 2 | chr1: 94, 540, 281 |
| B2M | 5 | CGUGAGUAAACCUGAAUCUU | TGG | Exon 2 | chr15: 44, 715, 434 |
| CIITA | 129 | GAUAUUGGCAUAAGCCUCCC | TGG | Exon 3 | chr16: 10, 895, 747 |
| TRAC | 130 | AGAGUCUCUCAGCUGGUACA | CGG | Exon 1 | chr14: 22, 5547, 533 |
| CD52 | 94 | CAGCCTCCTGGTTATGGTAC | | Exon 1 | |
| CD70 | 95 | GCTACGTATCCATCGTGA | | Exon 3 | |
| | 96 | GTACACATCCAGGTGACGC | | | |
| | 97 | GCAGGCTGATGCTACGGG | | | |
| | 98 | TCACCAAGCCCGCGACCAAT | | | |
| CD70 | 99 | TCACCAAGCCCGCGACCAAT | GGG | Exon 1 | |
| | 100 | ATCACCAAGCCCGCGACCAA | TGG | | |
| | 101 | CGGTGCGGCGCAGGCCCTAT | GGG | | |
| | 102 | GCTTTGGTCCCATTGGTCGC | GGG | | |
| | 103 | GCCCGCAGGACGCACCCATA | GGG | | |
| | 104 | GTGCATCCAGCGCTTCGCAC | AGG | | |
| | 105 | CAGCTACGTATCCATCGTGA | TGG | | |
| CD155 | 106 | GGACAAAGGCGCAAGTCGAG | | | |

For the Cas9 guides, the spacer sequence for all Cas9 guides is provided in Table 38, with description that the 20nt guide sequence corresponds to a unique guide sequence and can be any of those described herein, including for example those listed in Table 37.

TABLE 38
Cas9 guide RNAs

| | SEQ ID | |
|---|---|---|
| Description | NO: | Sequence |
| 20 nt guide | 131 | NNNNNNNNNNNNNNNNNNNN |
| sequence* | | |
| 12 nt crRNA repeat | 132 | GUUUUAGAGCUA |
| sequence | | |
| 4 nt tetraloop | 133 | GAAA |
| sequence | | |
| 64 nt tracrRNA | 134 | UAGCAAGUUAAAAUAAGGCUAGUCCGUUAUCAACUUGAAAAAGUGG |
| sequence | | CACCGAGUCGGUGCUUU |
| Exemplary full | 135 | NNNNNNNNNNNNNNNNNNNNGUUUUAGAGCUAGAAAUAGCAAGU |
| sequence | | UAAAAUAAGGCUAGUCCGUUAUCAACUUGAAAAAGUGGCACCGAGU |
| | | CGGUGCUUU |

In some embodiments, the hypoimmunogenic cell that includes the exogenous polynucleotide is derived from a HIP cell, for example, as described herein. Such hypoimmunogenic cells include, for example, T cells and NK cells. In some embodiments, the hypoimmunogenic cell that includes the exogenous polynucleotide is a T cell (e.g., a primary T cell), or an NK cell.

In some embodiments, the exogenous polynucleotide encodes an exogenous CD47 polypeptide (e.g., a human CD47 polypeptide) and the exogenous polypeptide is inserted into the genome of the cell using a gene therapy vector. In some embodiments, the exogenous polynucleotide encodes an exogenous CD47 polypeptide (e.g., a human CD47 polypeptide) and the exogenous polypeptide is inserted into a safe harbor or target gene loci or a safe harbor or target site as disclosed herein or a genomic locus that causes silencing or reduced expression of the endogenous gene. In some embodiments, the polynucleotide is inserted in a B2M, CIITA, TRAC, TRB, PD1 or CTLA4 gene locus.

In some embodiments, the hypoimmunogenic cell that includes the exogenous polynucleotide is a primary T cell or a T cell derived from a hypoimmunogenic pluripotent cell (e.g., a hypoimmunogenic iPSC). In exemplary embodiments, the exogenous polynucleotide is a chimeric antigen receptor (e.g., any of the CARs described herein). In some embodiments, the exogenous polynucleotide is operably linked to a promoter for expression of the exogenous polynucleotide in the hypoimmunogenic cell.

### M. Methods of Producing Hypoimmungenic Cells

The technology provides methods of producing hypoimmunogenic pluripotent cells. In some embodiments, the method comprises generating pluripotent stem cells. The generation of mouse and human pluripotent stem cells (generally referred to as iPSCs; miPSCs for murine cells or hiPSCs for human cells) is generally known in the art. As will be appreciated by those in the art, there are a variety of different methods for the generation of iPCSs. The original induction was done from mouse embryonic or adult fibroblasts using the viral introduction of four transcription factors, Oct3/4, Sox2, c-Myc and Klf4; see Takahashi and Yamanaka Cell 126:663-676 (2006), hereby incorporated by reference in its entirety and specifically for the techniques outlined therein. Since then, a number of methods have been developed; see Seki et al, World J. Stem Cells 7(1): 116-125 (2015) for a review, and Lakshmipathy and Vermuri, editors, Methods in Molecular Biology: Pluripotent Stem Cells, Methods and Protocols, Springer 2013, both of which are hereby expressly incorporated by reference in their entirety, and in particular for the methods for generating hiPSCs (see for example Chapter 3 of the latter reference).

Generally, iPSCs are generated by the transient expression of one or more reprogramming factors″ in the host cell, usually introduced using episomal vectors. Under these conditions, small amounts of the cells are induced to become iPSCs (in general, the efficiency of this step is low, as no selection markers are used). Once the cells are “reprogrammed”, and become pluripotent, they lose the episomal vector(s) and produce the factors using the endogenous genes.

As is also appreciated by those of skill in the art, the number of reprogramming factors that can be used or are used can vary. Commonly, when fewer reprogramming factors are used, the efficiency of the transformation of the cells to a pluripotent state goes down, as well as the “pluripotency”, e.g., fewer reprogramming factors may result in cells that are not fully pluripotent but may only be able to differentiate into fewer cell types.

In some embodiments, a single reprogramming factor, OCT4, is used. In other embodiments, two reprogramming factors, OCT4 and KLF4, are used. In other embodiments, three reprogramming factors, OCT4, KLF4 and SOX2, are used. In other embodiments, four reprogramming factors, OCT4, KLF4, SOX2 and c-Myc, are used. In other embodiments, 5, 6 or 7 reprogramming factors can be used selected from SOKMNLT; SOX2, OCT4 (POU5F1), KLF4, MYC, NANOG, LIN28, and SV40L T antigen. In general, these reprogramming factor genes are provided on episomal vectors such as are known in the art and commercially available.

In general, as is known in the art, iPSCs are made from non-pluripotent cells such as, but not limited to, blood cells, fibroblasts, etc., by transiently expressing the reprogramming factors as described herein.

### Assays for Hypoimmunogenicity Phenotypes and Retention of Pluripotency

Once the engineered CAR-T cells have been generated, they may be assayed for their hypoimmunogenicity and/or retention of pluripotency as is described in WO2016183041 and WO2018132783.

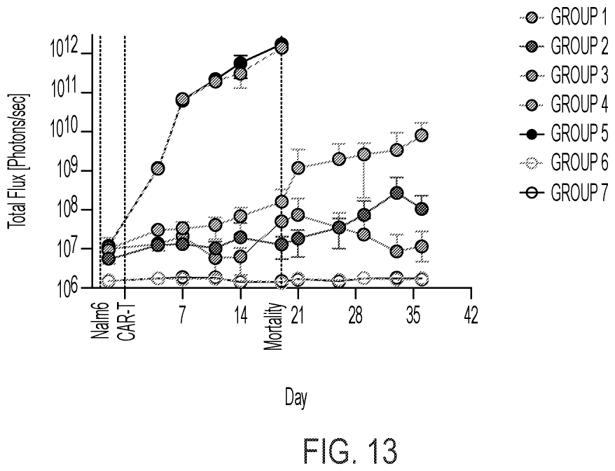

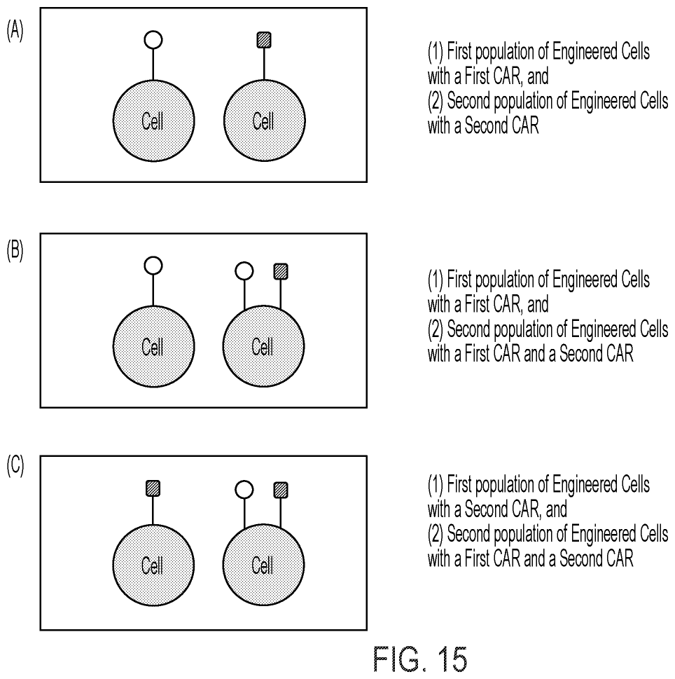

In some embodiments, hypoimmunogenicity is assayed using a number of techniques as exemplified in FIG. 13 and FIG. 15 of WO2018132783. These techniques include transplantation into allogeneic hosts and monitoring for hypoimmunogenic pluripotent cell growth (e.g., teratomas) that escape the host immune system. In some instances, hypoimmunogenic pluripotent cell derivatives are transduced to express luciferase and can then followed using bioluminescence imaging. Similarly, the T cell and/or B cell response of the host animal to such cells are tested to confirm that the cells do not cause an immune reaction in the host animal. T cell responses can be assessed by Elispot, ELISA, FACS, PCR, or mass cytometry (CYTOF). B cell responses or antibody responses are assessed using FACS or Luminex. Additionally or alternatively, the cells may be assayed for their ability to avoid innate immune responses, e.g., NK cell killing, as is generally shown in FIGS. 14 and 15 of WO2018132783.

In some embodiments, the immunogenicity of the cells is evaluated using T cell immunoassays such as T cell proliferation assays, T cell activation assays, and T cell killing assays recognized by those skilled in the art. In some cases, the T cell proliferation assay includes pretreating the cells with interferon-gamma and coculturing the cells with labelled T cells and assaying the presence of the T cell population (or the proliferating T cell population) after a preselected amount of time. In some cases, the T cell activation assay includes coculturing T cells with the cells outlined herein and determining the expression levels of T cell activation markers in the T cells.

In vivo assays can be performed to assess the immunogenicity of the cells outlined herein. In some embodiments, the survival and immunogenicity of hypoimmunogenic cells is determined using an allogenic humanized immunodeficient mouse model. In some instances, the hypoimmunogenic pluripotent stem cells are transplanted into an allogenic humanized NSG-SGM3 mouse and assayed for cell rejection, cell survival, and teratoma formation. In some instances, grafted hypoimmunogenic pluripotent stem cells or differentiated cells thereof display long-term survival in the mouse model.

Additional techniques for determining immunogenicity including hypoimmunogenicity of the cells are described in, for example, Deuse et al., Nature Biotechnology, 2019, 37, 252-258 and Han et al., Proc Natl Acad Sci USA, 2019, 116(21), 10441-10446, the disclosures including the figures, figure legends, and description of methods are incorporated herein by reference in their entirety.

Similarly, the retention of pluripotency is tested in a number of ways. In some embodiments, pluripotency is assayed by the expression of certain pluripotency-specific factors as generally described herein and shown in FIG. 29 of WO2018132783. Additionally or alternatively, the pluripotent cells are differentiated into one or more cell types as an indication of pluripotency.

As will be appreciated by those in the art, the successful reduction of the MHC I function (HLA I when the cells are derived from human cells) in the pluripotent cells can be measured using techniques known in the art and as described below; for example, FACS techniques using labeled antibodies that bind the HLA complex; for example, using commercially available HLA-A, HLA-B, and HLA-C antibodies that bind to the alpha chain of the human major histocompatibility HLA Class I antigens.

In addition, the cells can be tested to confirm that the HLA I complex is not expressed on the cell surface. This may be assayed by FACS analysis using antibodies to one or more HLA cell surface components as discussed above.

The successful reduction of the MHC II function (HLA II when the cells are derived from human cells) in the pluripotent cells or their derivatives can be measured using techniques known in the art such as Western blotting using antibodies to the protein, FACS techniques, RT-PCR techniques, etc.

In addition, the cells can be tested to confirm that the HLA 11 complex is not expressed on the cell surface. Again, this assay is done as is known in the art (See FIG. 21 of WO2018132783, for example) and generally is done using either Western Blots or FACS analysis based on commercial antibodies that bind to human HLA Class II HLA-DR, DP and most DQ antigens.

In addition to the reduction of HLA I and II (or MHC I and II), the engineered CAR-T cells of the technology have a reduced susceptibility to macrophage phagocytosis and NK cell killing. The resulting hypoimmunogenic cells “escape” the immune macrophage and innate pathways due to reduction or lack of the TCR complex and the expression of one or more CD47 transgenes.

### N. Assays for Hypoimmunogenicity Phenotypes and Retention of Pluripotency

Once the engineered CAR-T cells have been generated, they may be assayed for their hypoimmunogenicity and/or retention of pluripotency as is described in WO2016183041 and WO2018132783.

In some embodiments, hypoimmunogenicity is assayed using a number of techniques as exemplified in FIG. 13 and FIG. 15 of WO2018132783. These techniques include transplantation into allogeneic hosts and monitoring for hypoimmunogenic pluripotent cell growth (e.g., teratomas) that escape the host immune system. In some instances, hypoimmunogenic pluripotent cell derivatives are transduced to express luciferase and can then followed using bioluminescence imaging. Similarly, the T cell and/or B cell response of the host animal to such cells are tested to confirm that the cells do not cause an immune reaction in the host animal. T cell responses can be assessed by Elispot, ELISA, FACS, PCR, or mass cytometry (CYTOF). B cell responses or antibody responses are assessed using FACS or Luminex. Additionally or alternatively, the cells may be assayed for their ability to avoid innate immune responses, e.g., NK cell killing, as is generally shown in FIGS. 14 and 15 of WO2018132783.

In some embodiments, the immunogenicity of the cells is evaluated using T cell immunoassays such as T cell proliferation assays, T cell activation assays, and T cell killing assays recognized by those skilled in the art. In some cases, the T cell proliferation assay includes pretreating the cells with interferon-gamma and coculturing the cells with labelled T cells and assaying the presence of the T cell population (or the proliferating T cell population) after a preselected amount of time. In some cases, the T cell activation assay includes coculturing T cells with the cells outlined herein and determining the expression levels of T cell activation markers in the T cells.

In vivo assays can be performed to assess the immunogenicity of the cells outlined herein. In some embodiments, the survival and immunogenicity of hypoimmunogenic cells is determined using an allogenic humanized immunodeficient mouse model. In some instances, the hypoimmunogenic pluripotent stem cells are transplanted into an allogenic humanized NSG-SGM3 mouse and assayed for cell rejection, cell survival, and teratoma formation. In some instances, grafted hypoimmunogenic pluripotent stem cells or differentiated cells thereof display long-term survival in the mouse model.

Additional techniques for determining immunogenicity including hypoimmunogenicity of the cells are described in, for example, Deuse et al., Nature Biotechnology, 2019, 37, 252-258 and Han et al., Proc Natl Acad Sci USA, 2019, 116(21), 10441-10446, the disclosures including the figures, figure legends, and description of methods are incorporated herein by reference in their entirety.

Similarly, the retention of pluripotency is tested in a number of ways. In some embodiments, pluripotency is assayed by the expression of certain pluripotency-specific factors as generally described herein and shown in FIG. 29 of WO2018132783. Additionally or alternatively, the pluripotent cells are differentiated into one or more cell types as an indication of pluripotency.

As will be appreciated by those in the art, the successful reduction of the MHC I function (HLA I when the cells are derived from human cells) in the pluripotent cells can be measured using techniques known in the art and as described below; for example, FACS techniques using labeled antibodies that bind the HLA complex; for example, using commercially available HLA-A, HLA-B, and HLA-C antibodies that bind to the alpha chain of the human major histocompatibility HLA Class I antigens.

In addition, the cells can be tested to confirm that the HLA I complex is not expressed on the cell surface. This may be assayed by FACS analysis using antibodies to one or more HLA cell surface components as discussed above.

The successful reduction of the MHC II function (HLA II when the cells are derived from human cells) in the pluripotent cells or their derivatives can be measured using techniques known in the art such as Western blotting using antibodies to the protein, FACS techniques, RT-PCR techniques, etc.

In addition, the cells can be tested to confirm that the HLA 11 complex is not expressed on the cell surface. Again, this assay is done as is known in the art (See FIG. 21 of WO2018132783, for example) and generally is done using either Western Blots or FACS analysis based on commercial antibodies that bind to human HLA Class II HLA-DR, DP and most DQ antigens.

In addition to the reduction of HLA I and II (or MHC I and II), the engineered CAR-T cells of the technology have a reduced susceptibility to macrophage phagocytosis and NK cell killing. The resulting hypoimmunogenic cells “escape” the immune macrophage and innate pathways due to reduction or lack of the TCR complex and the expression of one or more CD47 transgenes.

### O. Pharmaceutical Compositions

### 1. Pharmaceutically Acceptable Carriers

In some embodiments, the pharmaceutical composition provided herein further include a pharmaceutically acceptable carrier. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); salts such as sodium chloride; and/or non-ionic surfactants such as polysorbates (TWEEN™), poloxamers (PLURONICS™) or polyethylene glycol (PEG). In some embodiments, the pharmaceutical composition includes a pharmaceutically acceptable buffer (e.g., neutral buffer saline or phosphate buffered saline).

In some embodiments, the pharmaceutical composition includes one or more electrolyte base solutions selected from the group consisting of lactated CryoStor®, Ringer's solution, PlasmaLyte-A™, Iscove's Modified Dulbecco's Medium, Normosol-R™, Veen-D™, Polysal® and Hank's Balanced Salt Solution (containing no phenol red). These base solutions closely approximate the composition of extracellular mammalian physiological fluids.

In some embodiments, the pharmaceutical composition includes one or more cryoprotective agents selected from the group consisting of arabinogalactan, glycerol, polyvinylpyrrolidone (PVP), dextrose, dextran, trehalose, sucrose, raffinose, hydroxyethyl starch (HES), propylene glycol, human serum albumin (HSA), and dimethylsulfoxide (DMSO). In some embodiments, the pharmaceutically acceptable buffer is neutral buffer saline or phosphate buffered saline. In some embodiments, pharmaceutical compositions provided herein include one or more of CryoStor® CSB, Plasma-Lyte-A™, HSA, DMSO, and trehalose.

CryoStor® is an intracellular-like optimized solution containing osmotic/oncotic agents, free radical scavengers, and energy sources to minimize apoptosis, minimize ischemia/reperfusion injury and maximize the post-thaw recovery of the greatest numbers of viable, functional cells. CryoStor® is serum- and protein-free, and non-immunogenic. CryoStor® is cGMP-manufactured from raw materials of USPgrade or higher. CryoStor® is a family of solutions pre-formulated with 0%, 2%, 5% or 10% DMSO. CryoStor® CSB is a DMSO-free version of CryoStor®. In some embodiments, the pharmaceutical composition includes a base solution of CryoStor® CSB at a concentration of about 0-100%, 5-95%, 10-90%, 15-85%, 20-80%, 30-80%, 40-80%, 50-80%, 60-80%, 70-80%, 25-75%, 30-70%, 35-65%, 40-60%, or 45-55% w/w. In some embodiments, the pharmaceutical composition includes a base solution of CryoStor® CSB at a concentration of about 0%, 5%, 10%, 15%, 20%, 25%, 30%, 35%, 40%, 45%, 50%, 55%, 60%, 65%, 70%, 75%, 80%, 85%, 90%, 95%, or 100% w/w.

PlasmaLyte-A™ is a non-polymeric plasma expander and contains essential salts and nutrients similar to those found in culture medium but does not contain additional constituents found in tissue culture medium which are not approved for human infusion, e.g., phenol red, or are unavailable in U.S.P. grade. PlasmaLyte-AT^{M} contains about 140 mEq/liter of sodium (Na), about 5 mEq/liter of potassium (K), about 3 mEq/liter of magnesium (Mg), about 98 mEq/liter of chloride (CI), about 27 mEq/liter of acetate, and about 23 mEq/liter of gluconate. (PlasmaLyte-AT^{M} is commercially available from Baxter, Hyland Division, Glendale Calif., product No. 2B2543). In some embodiments, the pharmaceutical composition includes a base solution of PlasmaLyte-A™ at a concentration of about 0-100%, 5-95%, 10-90%, 15-85%, 15-80%, 15-75%, 15-70%, 15-65%, 15-60%, 15-55%, 15-50%, 15-45%, 15-40%, 15-35%, 15-30%, 15-25%, 20-80%, 20-75%, 20-70%, 20-65%, 20-60%, 20-55%, 20-50%, 20-45%, 20-40%, 20-35%, 20-30%, 25-75%, 30-70%, 35-65%, 40-60%, or 45-55% w/w. In some embodiments, the pharmaceutical composition includes a base solution of PlasmaLyte-A™ at a concentration of about 0%, 5%, 10%, 15%, 20%, 25%, 30%, 35%, 40%, 45%, 50%, 55%, 60%, 65%, 70%, 75%, 80%, 85%, 90%, 95%, or 100% w/w.

In some embodiments, the pharmaceutical composition includes human serum albumin (HSA) at a concentration of about 0-10%, 0.3-9.3%, 0.3-8.3%, 0.3-7.3%, 0.3-6.3%, 0.3-5.3%, 0.3-4.3%, 0.3-3.3%, 0.3-2.3%, 0.3-1.3%, 0.6-8.3%, 0.9-7.3%, 1.2-6.3%, 1.5-5.3%, 1.8-4.3%, or 2.1-3.3% w/v. In some embodiments, the pharmaceutical composition includes HSA at a concentration of about 0%, 0.3%, 0.6%, 0.9%, 1.2%, 1.5%, 1.8%, 2.1%, 2.4%, 2.7%, 3.0%, 3.3%, 3.6%, 3.9%, 4.3%, 4.6%, 4.9%, 5.3%, 5.6%, 5.9%, 6.3%, 6.6%, 6.9%, 7.3%, 7.6%, 7.9%, 8.3%, 8.6%, 8.9%, 9.3%, 9.6%, 9.9%, or 10% w/v.

In some embodiments, the pharmaceutical composition includes DMSO at a concentration of about 0-10%, 0.5-9.5%, 1-9%, 1.5-8.5%, 2-8%, 3-8%, 4-8%, 5-8%, 6-8%, 7-8%, 2.5-7.5%, 3- 7%, 3.5-6.5%, 4-6%, or 4.5-5.5% v/v. In some embodiments, the pharmaceutical composition includes HSA at a concentration of about 0%, 0.25%, 0.5%, 0.75%, 1.0%, 1.25%, 1.5%, 1.75%, 2.0%, 2.25%, 2.5%, 2.75%, 3.0%, 3.25%, 3.5%, 3.75%, 4.0%, 4.25%, 4.5%, 4.75%, 5.0%, 5.25%, 5.5%, 5.75%, 6.0%, 6.25%, 6.5%, 6.75%, 7.0%, 7.25%, 7.5%, 7.75%, 8.0%, 8.25%, 8.5%, 8.75%, 9.0%, 9.25%, 9.5%, 9.75%, or 10.0% v/v.

In some embodiments, the pharmaceutical composition includes trehalose at a concentration of about 0-500 mM, 50-450 mM, 100-400 mM, 150-350 mM, or 200-300 mM. In some embodiments, the pharmaceutical composition includes trehalose at a concentration of about 0 mM, 10 mM, 20 mM, 30 mM, 40 mM, 50 mM, 60 mM, 70 mM, 80 mM, 90 mM, 100 mM, 125 mM, 150 mM, 175 mM, 200 mM, 225 mM, 250 mM, 275 mM, 300 mM, 325 mM, 350 mM, 375 mM, 400 mM, 425 mM, 450 mM, 475 mM, or 500 mM.

Exemplary pharmaceutical composition components are shown in Table 39.

TABLE 39
Exemplary pharmaceutical composition components.

| Formu- | | | Additional | |
|---|---|---|---|---|
| lation | Base Solution | c[DMSO] | c[HSA]* | c[trehalose] |
| A | 75% CroStor ® CSB + | 7.5% | 0.3% | |
| B | 25% PlasmaLyte | 3.75% | 0.3% | |
| C | A ™ + 1.2% HSA | | 5.3% | |
| D | | | 0.3% | 250 mM |
| E | 100% PlasmaLyte | 7.5% | 0.3% | |
| F | A ™ + 1.2% HSA | 7.5% | 5.3% | |
| G | | 7.5% | 5.3% | 250 mM |

*Additional HSA in addition to PlasmaLyte.

In some embodiments, the pharmaceutical composition comprises hypoimmunogenic cells described herein and a pharmaceutically acceptable carrier comprising 31.25% (v/v) Plasma-Lyte A, 31.25% (v/v) of 5% dextrose/0.45% sodium chloride, 10% dextran 40 (LMD)/5% dextrose, 20% (v/v) of 25% human serum albumin (HSA), and 7.5% (v/v) dimethylsulfoxide (DMSO).

### 2. Formulations and Dosage Regimens

Any therapeutically effective amount of cells described herein can be included in the pharmaceutical composition, depending on the indication being treated. Non-limiting examples of the cells include primary T cells, T cells differentiated from hypoimmunogenic induced pluripotent stem cells, and other cells differentiated from hypoimmunogenic induced pluripotent stem cells described herein. In some embodiments, the pharmaceutical composition includes at least about 1×10², 5×10², 1×10³, 5×10³, 1×10⁴, 5×10⁴, 1×10⁶, 5×10⁶, 1×10⁶, 5×10⁶, 1×10⁷, 5×10⁸, 1×10⁸, 5×10⁸, 1×10⁹, 5×10⁹, 1×10¹⁰, or 5×10¹⁰ cells. In some embodiments, the pharmaceutical composition includes up to about 1×10², 5×10², 1×10³, 5×10³, 1×10⁴, 5×10⁴, 1×10⁶, 5×10⁶, 1×10⁶, 5×10⁶, 1×10⁷, 5×10⁷, 1×10⁸, 5×10⁸, 1×10⁹, 5×10⁹, 1×10¹⁰, or 5×10¹⁰ cells. In some embodiments, the pharmaceutical composition includes up to about 6.0×10⁸ cells. In some embodiments, the pharmaceutical composition includes up to about 8.0×10⁸ cells. In some embodiments, the pharmaceutical composition includes at least about 1×10²-5×10², 5×10²-1×10³, 1×10³-5×10³, 5×10³-1×10⁴, 1×10⁴-5×10⁴, 5×10⁴-1×10⁵, 1×10⁵-5×10⁶, 5×10⁵-1×10⁶, 1×10⁶-5×10⁶, 5×10⁶-1×10⁸, 1×10⁷-5×10⁸, 5×10⁷-1×10⁸, 1×10⁸-5×10⁸, 5×10⁸-1×10⁹, 1×10⁹-5×10¹⁰, 5×10⁹-1×10¹⁰, or 1×10¹⁰-5×10¹⁰ cells. In exemplary embodiments, the pharmaceutical composition includes from about 1.0×10⁶ to about 2.5×10⁸ cells. In certain embodiments, the pharmaceutical composition includes from about 2.0×10⁶ to about 5.0×10⁸ cells, such as but not limited to, primary T cells, T cells differentiated from hypoimmunogenic induced pluripotent stem cells. In some embodiments, the pharmaceutical composition includes about the same number of CAR-T cells as were included in the prior CD19-CAR-T pharmaceutical composition. In some embodiments, the pharmaceutical composition includes more or a greater number of CAR-T cells than were included in the prior CD19-CAR-T pharmaceutical composition. In some embodiments, the pharmaceutical composition includes fewer or a lower number of CAR-T cells than were included in the prior CD19-CAR-T pharmaceutical composition.

In some embodiments, the pharmaceutical composition has a volume of at least 5, 10, 15, 20, 25, 30, 35, 40, 45, 50, 55, 60, 65, 70, 75, 80, 85, 90, 95, 100, 110, 120, 130, 140, 150, 160, 170, 180, 190, 200, 250, 300, 350, 400, or 500 ml. In exemplary embodiments, the pharmaceutical composition has a volume of up to about 5, 10, 15, 20, 25, 30, 35, 40, 45, 50, 55, 60, 65, 70, 75, 80, 85, 90, 95, 100, 110, 120, 130, 140, 150, 160, 170, 180, 190, 200, 250, 300, 350, 400, or 500 ml. In exemplary embodiments, the pharmaceutical composition has a volume of about 5, 10, 15, 20, 25, 30, 35, 40, 45, 50, 55, 60, 65, 70, 75, 80, 85, 90, 95, 100, 110, 120, 130, 140, 150, 160, 170, 180, 190, 200, 250, 300, 350, 400, or 500 ml. In some embodiments, the pharmaceutical composition has a volume of from about 1-50 ml, 50-100 ml, 100-150 ml, 150-200 ml, 200-250 ml, 250-300 ml, 300-350 ml, 350-400 ml, 400-450 ml, or 450-500 ml. In some embodiments, the pharmaceutical composition has a volume of from about 1-50 ml, 50-100 ml, 100-150 ml, 150-200 ml, 200-250 ml, 250-300 ml, 300-350 ml, 350-400 ml, 400-450 ml, or 450-500 ml. In some embodiments, the pharmaceutical composition has a volume of from about 1-10 ml, 10-20 ml, 20-30 ml, 30-40 ml, 40-50 ml, 50-60 ml, 60-70 ml, 70-80 ml, 70-80 ml, 80-90 ml, or 90-100 ml. In some embodiments, the pharmaceutical composition has a volume that ranges from about 5 ml to about 80 ml. In exemplary embodiments, the pharmaceutical composition has a volume that ranges from about 10 ml to about 70 ml. In certain embodiments, the pharmaceutical composition has a volume that ranges from about 10 ml to about 50 ml.

The specific amount/dosage regimen will vary depending on the weight, gender, age and health of the individual; the formulation, the biochemical nature, bioactivity, bioavailability and the side effects of the cells and the number and identity of the cells in the complete therapeutic regimen.

In some embodiments, a therapeutically effective dose or a clinically effective dose of the pharmaceutical composition includes about 1.0×10⁵ to about 2.5×10⁸ cells at a volume of about 10 ml to 50 ml and the pharmaceutical composition is administered as a single therapeutically effective dose or clinically effective dose. In some cases, the therapeutically effective dose or clinically effective dose includes about 1.0×10⁵ to about 2.5×10⁸ primary T cells described herein at a volume of about 10 ml to 50 ml. In some cases, the therapeutically effective dose or clinically effective dose includes about 1.0×105 to about 2.5×10⁸ primary T cells that have been described above at a volume of about 10 ml to 50 ml. In various cases, the therapeutically effective dose or clinically effective dose includes about 1.0×105 to about 2.5×10⁸ T cells differentiated from hypoimmunogenic induced pluripotent stem cells described herein at a volume of about 10 ml to 50 ml. In some embodiments, the therapeutically effective dose or clinically effective dose is 1.0×10⁵, 1.1×10⁵, 1.2×10⁵, 1.3×10⁵, 1.4×10⁵, 1.5×10⁵, 1.6×10⁶, 1.7×10⁵, 1.8×10⁵, 1.9×10⁶, 2.0×10⁵, 2.1×10⁶, 2.2×10⁶, 2.3×10⁶, 2.4×10⁶, 2.5×10⁵, 1.0×10⁶, 1.1×10⁶, 1.2×10⁶, 1.3×10⁶, 1.4×10⁶, 1.5×10⁶, 1.6×10⁶, 1.7×10⁶, 1.8×10⁶, 1.9×10⁶, 2.0×10⁶, 2.1×10⁶, 2.2×10⁶, 2.3×10⁶, 2.4×10⁶, 2.5×10⁶, 1.0×10⁷, 1.1×10⁷, 1.2×10⁷, 1.3×10⁷, 1.4×10⁷, 1.5×10⁷, 1.6×10⁷, 1.7×10⁷, 1.8×10⁷, 1.9×10⁷, 2.0×10⁷, 2.1×10⁷, 2.2×10⁷, 2.3×10⁷, 2.4×10⁷, 2.5×10⁷, 1.0×10⁸, 1.1×10⁸, 1.2×10⁸, 1.3×10⁸, 1.4×10⁸, 1.5×10⁸, 1.6×10⁸, 1.7×10⁸, 1.8×10⁸, 1.9×10⁸, 2.0×10⁸, 2.1×10⁸, 2.2×10⁸, 2.3×10⁸, 2.4×10⁸, or 2.5×10⁸ T cells differentiated from hypoimmunogenic induced pluripotent stem cells described herein at a volume of about 10 ml to 50 ml. In other cases, the therapeutically effective dose or clinically effective dose is at a range that is lower than about 1.0×10⁵ to about 2.5×10⁸ T cells, including primary T cells or T cells differentiated from hypoimmunogenic induced pluripotent stem cells. In yet other cases, the therapeutically effective dose or clinically effective dose is at a range that is higher than about 1.0×10⁵ to about 2.5×10⁸ T cells, including primary T cells and T cells differentiated from hypoimmunogenic induced pluripotent stem cells.

In some embodiments, the pharmaceutical composition is administered as a single therapeutically effective dose or clinically effective dose of from about 1.0×10⁵ to about 1.0×10⁷ cells (such as primary T cells and T cells differentiated from hypoimmunogenic induced pluripotent stem cells) per kg body weight for subjects 50 kg or less. In some embodiments, the pharmaceutical composition is administered as a single therapeutically effective dose or clinically effective dose of from about 0.5×10⁵ to about 1.0×10⁷, about 1.0×10⁵ to about 1.0×10⁷, about 1.0×10⁵ to about 1.0×10⁷, about 5.0×10⁵ to about 1×10⁷, about 1.0×10⁶ to about 1×10⁷, about 5.0×10⁶ to about 1.0×10⁷, about 1.0×10⁵ to about 5.0×10⁶, about 1.0×10⁵ to about 1.0×10⁶, about 1.0×10⁵ to about 5.0×10⁵, about 1.0×10⁵ to about 5.0×10⁶, about 2.0×10⁵ to about 5.0×10⁶, about 3.0×10⁵ to about 5.0×10⁶, about 4.0×10⁵ to about 5.0×10⁶, about 5.0×10⁵ to about 5.0×10⁶, about 6.0×10⁵ to about 5.0×10⁶, about 7.0×10⁵ to about 5.0×10⁶, about 8.0×10⁵ to about 5.0×10⁶, or about 9.0×10⁵ to about 5.0×10⁶ cells per kg body weight for subjects 50 kg or less. In some embodiments, the therapeutically effective dose or clinically effective dose is 0.5×10⁵, 0.6×10⁵, 0.7×10⁵, 0.8×10⁵, 0.9×10⁵, 1.0×10⁵, 1.1×10⁶, 1.2×10⁵, 1.3×10⁵, 1.4×10⁵, 1.5×10⁵, 1.6×10⁵, 1.7×10⁵, 1.8×10⁵, 1.9×10⁶, 2.0×10⁶, 2.1×10⁶, 2.2×10⁵, 2.3×10⁵, 2.4×10⁵, 2.5×10⁵, 2.6×10⁵, 2.7×10⁵, 2.8×10⁵, 2.9×10⁵, 3.0×10⁵, 3.1×10⁵, 3.2×10⁶, 3.3×10⁶, 3.4×10⁶, 3.5×10⁶, 3.6×10⁶, 3.7×10⁶, 3.8×10⁶, 3.9×10⁶, 4.0×10⁶, 4.1×10⁶, 4.2×10⁶, 4.3×10⁵, 4.4×10⁵, 4.5×10⁵, 4.6×10⁵, 4.7×10⁵, 4.8×10⁵, 4.9×10⁵, 5.0×10⁵, 0.5×10⁶, 0.6×10⁶, 0.7×10⁶, 0.8×10⁶, 0.9×10⁶, 1.0×10⁶, 1.1×10⁶, 1.2×10⁶, 1.3×10⁶, 1.4×10⁶, 1.5×10⁶, 1.6×10⁶, 1.7×10⁶, 1.8×10⁶, 1.9×10⁶, 2.0×10⁶, 2.1×10⁶, 2.2×10⁶, 2.3×10⁶, 2.4×10⁶, 2.5×10⁶, 2.6×10⁶, 2.7×10⁶, 2.8×10⁶, 2.9×10⁶, 3.0×10⁶, 3.1×10⁶, 3.2×10⁶, 3.3×10⁶, 3.4×10⁶, 3.5×10⁶, 3.6×10⁶, 3.7×10⁶, 3.8×10⁶, 3.9×10⁶, 4.0×10⁶, 4.1×10⁶, 4.2×10⁶, 4.3×10⁶, 4.4×10⁶, 4.5×10⁶, 4.6×10⁶, 4.7×10⁶, 4.8×10⁶, 4.9×10⁶, 5.0×10⁶, 5.1×10⁶, 5.2×10⁶, 5.3×10⁶, 5.4×10⁶, 5.5×10⁶, 5.6×10⁶, 5.7×10⁶, 5.8×10⁶, 5.9×10⁶, 6.0×10⁶, 6.1×10⁶, 6.2×10⁶, 6.3×10⁶, 6.4×10⁶, 6.5×10⁶, 6.6×10⁶, 6.7×10⁶, 6.8×10⁶, 6.9×10⁶, 7.0×10⁶, 7.1×10⁶, 7.2×10⁶, 7.3×10⁶, 7.4×10⁶, 7.5×10⁶, 7.6×10⁶, 7.7×10⁶, 7.8×10⁶, 7.9×10⁶, 8.0×10⁶, 8.1×10⁶, 8.2×10⁶, 8.3×10⁶, 8.4×10⁶, 8.5×10⁶, 8.6×10⁶, 8.7×10⁶, 8.8×10⁶, 8.9×10⁶, 9.0×10⁶, 9.1×10⁶, 9.2×10⁶, 9.3×10⁶, 9.4×10⁶, 9.5×10⁶, 9.6×10⁶, 9.7×10⁶, 9.8×10⁶, 9.9×10⁶, 0.5×10⁸, 0.6×10⁸, 0.7×10⁷, 0.8×10⁷, 0.9×10⁷, or 1.0×10⁷ cells per kg body weight for subjects 50 kg or less. In some embodiments, the therapeutically effective dose or clinically effective dose is from about 0.2×10⁶ to about 5.0×10⁶ cells per kg body weight for subjects 50 kg or less. In certain embodiments, the therapeutically effective dose or clinically effective dose is at a range that is lower than from about 0.2×10⁶ to about 5.0×10⁶ cells per kg body weight for subjects 50 kg or less. or clinically effective dose. In exemplary embodiments, the single therapeutically effective dose or clinically effective dose is at a volume of about 10 ml to 50 ml. In some embodiments, the therapeutically effective dose or clinically effective dose is administered intravenously.

In exemplary embodiments, the cells are administered in a single therapeutically effective dose of from about 1.0×10⁶ to about 5.0×10⁸ cells (such as primary T cells and T cells differentiated from hypoimmunogenic induced pluripotent stem cells) for subjects above 50 kg. In some embodiments, the pharmaceutical composition is administered as a single therapeutically effective dose or clinically effective dose of from about 0.5×10⁶ to about 1.0×10⁹, about 1.0×10⁶ to about 1.0×10⁹, about 1.0×10⁶ to about 1.0×10⁹, about 5.0×10⁶ to about 1.0×10⁹, about 1.0×10⁷ to about 1.0×10⁹, about 5.0×107 to about 1.0×10⁹, about 1.0×10⁶ to about 5.0×10⁷, about 1.0×10⁶ to about 1.0×10⁷, about 1.0×10⁶ to about 5.0×10⁷, about 1.0×10⁷ to about 5.0×10⁸, about 2.0×10⁷ to about 5.0×10⁸, about 3.0×107 to about 5.0×10⁸, about 4.0×10⁷ to about 5.0×10⁸, about 5.0×10⁷ to about 5.0×10⁸, about 6.0×107 to about 5.0×10⁸, about 7.0×10⁷ to about 5.0×10⁸, about 8.0×10⁷ to about 5.0×10⁸, or about 9.0×107 to about 5.0×10⁸ cells per kg body weight for subjects 50 kg or less. In some embodiments, the therapeutically effective dose or clinically effective dose is 1.0×10⁶, 1.1×10⁶, 1.2×10⁶, 1.3×10⁶, 1.4×10⁶, 1.5×10⁶, 1.6×10⁶, 1.7×10⁶, 1.8×10⁶, 1.9×10⁶, 2.0×10⁶, 2.1×10⁶, 2.2×10⁶, 2.3×10⁶, 2.4×10⁶, 2.5×10⁶, 2.6×10⁶, 2.7×10⁶, 2.8×10⁶, 2.9×10⁶, 3.0×10⁶, 3.1×10⁶, 3.2×10⁶, 3.3×10⁶, 3.4×10⁶, 3.5×10⁶, 3.6×10⁶, 3.7×10⁶, 3.8×10⁶, 3.9×10⁶, 4.0×10⁶, 4.1×10⁶, 4.2×10⁶, 4.3×10⁶, 4.4×10⁶, 4.5×10⁶, 4.6×10⁶, 4.7×10⁶, 4.8×10⁶, 4.9×10⁶, 5.0×10⁶, 5.1×10⁶, 5.2×10⁶, 5.3×10⁶, 5.4×10⁶, 5.5×10⁶, 5.6×10⁶, 5.7×10⁶, 5.8×10⁶, 5.9×10⁶, 6.0×10⁶, 6.1×10⁶, 6.2×10⁶, 6.3×10⁶, 6.4×10⁶, 6.5×10⁶, 6.6×10⁶, 6.7×10⁶, 6.8×10⁶, 6.9×10⁶, 7.0×10⁶, 7.1×10⁶, 7.2×10⁶, 7.3×10⁶, 7.4×10⁶, 7.5×10⁶, 7.6×10⁶, 7.7×10⁶, 7.8×10⁶, 7.9×10⁶, 8.0×10⁶, 8.1×10⁶, 8.2×10⁶, 8.3×10⁶, 8.4×10⁶, 8.5×10⁶, 8.6×10⁶, 8.7×10⁶, 8.8×10⁶, 8.9×10⁶, 9.0×10⁶, 9.1×10⁶, 9.2×10⁶, 9.3×10⁶, 9.4×10⁶, 9.5×10⁶, 9.6×10⁶, 9.7×10⁶, 9.8×10⁶, 9.9×10⁶, 1.0×10⁷, 1.1×10⁷, 1.2×10⁷, 1.3×10⁷, 1.4×10⁷, 1.5×10⁷, 1.6×10⁷, 1.7×10⁷, 1.8×10⁷, 1.9×10⁷, 2.0×10⁷, 2.1×10⁷, 2.2×10⁷, 2.3×10⁷, 2.4×10⁷, 2.5×10⁷, 2.6×10⁷, 2.7×10⁷, 2.8×10⁷, 2.9×107, 3.0×10⁷, 3.1×10⁷, 3.2×10⁷, 3.3×10⁷, 3.4×10⁷, 3.5×10⁷, 3.6×10⁷, 3.7×10⁷, 3.8×10⁷, 3.9×10⁸, 4.0×10⁸, 4.1×10⁸, 4.2×10⁸, 4.3×10⁸, 4.4×10⁸, 4.5×10⁸, 4.6×10⁷, 4.7×10⁷, 4.8×10⁷, 4.9×10⁷, 5.0×107, 5.1×10⁷, 5.2×10⁷, 5.3×10⁷, 5.4×10⁷, 5.5×10⁷, 5.6×10⁷, 5.7×10⁷, 5.8×10⁷, 5.9×10⁷, 6.0×10⁸, 6.1×10⁸, 6.2×10⁸, 6.3×10⁸, 6.4×10⁸, 6.5×10⁸, 6.6×10⁸, 6.7×10⁷, 6.8×10⁷, 6.9×10⁷, 7.0×10⁷, 7.1×107, 7.2×10⁷, 7.3×10⁷, 7.4×10⁷, 7.5×10⁷, 7.6×10⁷, 7.7×10⁷, 7.8×10⁷, 7.9×10⁷, 8.0×10⁷, 8.1×10⁸, 8.2×10⁸, 8.3×10⁸, 8.4×10⁸, 8.5×10⁸, 8.6×10⁸, 8.7×10⁸, 8.8×10⁷, 8.9×10⁷, 9.0×10⁷, 9.1×10⁷, 9.2×107, 9.3×10⁷, 9.4×10⁷, 9.5×10⁷, 9.6×10⁷, 9.7×10⁷, 9.8×10⁷, 9.9×10⁷, 1.0×10⁸, 1.1×10⁸, 1.2×10⁸, 1.3×10⁸, 1.4×10⁸, 1.5×10⁸, 1.6×10⁸, 1.7×10⁸, 1.8×10⁸, 1.9×10⁸, 2.0×10⁸, 2.1×10⁸, 2.2×10⁸, 2.3×10⁸, 2.4×10⁸, 2.5×10⁸, 2.6×10⁸, 2.7×10⁸, 2.8×10⁸, 2.9×10⁸, 3.0×10⁸, 3.1×10⁸, 3.2×10⁸, 3.3×10⁸, 3.4×10⁸, 3.5×10⁸, 3.6×10⁸, 3.7×10⁸, 3.8×10⁸, 3.9×10⁸, 4.0×10⁸, 4.1×10⁸, 4.2×10⁸, 4.3×10⁸, 44×10⁸, 4.5×10⁸, 4.6×10⁸, 4.7×10⁸, 4.8×10⁸, 4.9×10⁸, or 5.0×10⁸ cells per kg body weight for subjects 50 kg or less. In certain embodiments, the cells are administered in a single therapeutically effective dose or clinically effective dose of about 1.0×10⁷ to about 2.5×10⁸ cells for subjects above 50 kg. In some embodiments, the cells are administered in a single therapeutically effective dose or clinically effective dose of a range that is less than about 1.0×10⁷ to about 2.5×10⁸ cells for subjects above 50 kg. In some embodiments, the cells are administered in a single therapeutically effective dose or clinically effective dose of a range that is higher than about 1.0×10⁷ to about 2.5×10⁸ cells for subjects above 50 kg. In some embodiments, the dose is administered intravenously. In exemplary embodiments, the single therapeutically effective dose or clinically effective dose is at a volume of about 10 ml to 50 ml. In some embodiments, the therapeutically effective dose or clinically effective dose is administered intravenously.

In exemplary embodiments, the therapeutically effective dose or clinically effective dose is administered intravenously at a rate of about 1 to 50 ml per minute, 1 to 40 ml per minute, 1 to 30 ml per minute, 1 to 20 ml per minute, 10 to 20 ml per minute, 10 to 30 ml per minute, 10 to 40 ml per minute, 10 to 50 ml per minute, 20 to 50 ml per minute, 30 to 50 ml per minute, 40 to 50 ml per minute. In numerous embodiments, the pharmaceutical composition is stored in one or more infusion bags for intravenous administration. In some embodiments, the dose is administered completely at no more than 10 minutes, 15 minutes, 20 minutes, 25 minutes, 30 minutes, 35 minutes, 40 minutes, 45 minutes, 50 minutes, 55 minutes, 60 minutes, 70 minutes, 80 minutes, 90 minutes, 120 minutes, 150 minutes, 180 minutes, 240 minutes, or 300 minutes.

In some embodiments, a single therapeutically effective dose or clinically effective dose of the pharmaceutical composition is present in a single infusion bag. In other embodiments, a single therapeutically effective dose or clinically effective dose of the pharmaceutical composition is divided into 2, 3, 4 or 5 separate infusion bags.

In some embodiments, the cells described herein are administered in a plurality of doses such as 2, 3, 4, 5, 6 or more doses, wherein the plurality of doses together constitute a therapeutically effective dose or clinically effective dose regimen. In some embodiments, each dose of the plurality of doses is administered to the subject ranging from 1 to 24 hours apart. In some instances, a subsequent dose is administered from about 1 hour to about 24 hours (e.g., about 1, 2, 3, 4, 5, 6, 7, 8, 9, 10, 11, 12, 13, 14, 15, 16, 17, 18, 19, 20, 21, 22, 23 or about 24 hours) after an initial or preceding dose. In some embodiments, each dose of the plurality of doses is administered to the subject ranging from about 1 day to 28 days apart. In some instances, a subsequent dose is administered from about 1 day to about 28 days (e.g., about 1, 2, 3, 4, 5, 6, 7, 8, 9, 10, 11, 12, 13, 14, 15, 16, 17, 18, 19, 20, 21, 22, 23, 24, 25, 26, 27, or about 28 days) after an initial or preceding dose. In certain embodiments, each dose of the plurality of doses is administered to the subject ranging from 1 week to about 6 weeks apart. In certain instances, a subsequent dose is administered from about 1 week to about 6 weeks (e.g., about 1, 2, 3, 4, 5, or 6 weeks) after an initial or preceding dose. In several embodiments, each dose of the plurality of doses is administered to the subject ranging from about 1 month to about 12 months apart. In several instances, a subsequent dose is administered from about 1 month to about 12 months (e.g., about 1, 2, 3, 4, 5, 6, 7, 8, 9, 10, 11, or 12 months) after an initial or preceding dose.

In some embodiments, the therapeutic dosing regimen of the CAR-T cells is the same as the therapeutic dosing regimen administered in the prior CD19-CAR-T therapy. In some embodiments, the therapeutic dosing regimen of the CAR-T cells is different from the therapeutic dosing regimen administered in the prior CD19-CAR-T therapy.

In some embodiments, a subject is administered a first dosage regimen at a first timepoint, and then subsequently administered a second dosage regimen at a second timepoint. In some embodiments, the first dosage regimen is the same as the second dosage regimen. In other embodiments, the first dosage regimen is different than the second dosage regimen. In some instances, the number of cells in the first dosage regimen and the second dosage regimen are the same. In some instances, the number of cells in the first dosage regimen and the second dosage regimen are different. In some cases, the number of doses of the first dosage regimen and the second dosage regimen are the same. In some cases, the number of doses of the first dosage regimen and the second dosage regimen are different.

In some embodiments, the first dosage regimen includes HIP T cells or primary T cells expressing a first CAR and the second dosage regimen includes HIP T cells or primary T cells expressing a second CAR such that the first CAR and the second CAR are different. For instance, the first CAR and second CAR bind different target antigens. In some cases, the first CAR includes an scFv that binds an antigen and the second CAR includes an scFv that binds a different antigen. In some embodiments, the first dosage regimen includes HIP T cell or primary T cells expressing a first CAR and the second dosage regimen includes HIP T cell or primary T cells expressing a second CAR such that the first CAR and the second CAR are the same. The first dosage regimen can be administered to the subject at least 1 month, 2 months, 3 months, 4 months, 5 months, 6 months, 7 months, 8 months, 9 months, 10 months, 11 months, 12 months, 1-3 months, 1-6 months, 4-6 months, 3-9 months, 3-12 months, or more months apart from the second dosage regimen. In some embodiments, a subject is administered a plurality of dosage regimens during the course of a disease (e.g., cancer) and at least two of the dosage regimens comprise the same type of HIP T cells or primary T cells described herein. In other embodiments, at least two of the plurality of dosage regimens comprise different types of HIP T cells or primary T cells described herein.

In some embodiments, the CD22-specific CAR-T cells described herein are administered to a subject at a dose of about 50×10⁶ to about 110×10⁶ (e.g., 50×10⁶, 51×10⁶, 52×10⁶, 53×10⁶, 54×10⁶, 55×10⁶, 56×10⁶, 57×10⁶, 58×10⁶, 59×10⁶, 60×10⁶, 61×10⁶, 62×10⁶, 63×10⁶, 64×10⁶, 65×10⁶, 66×10⁶, 67×10⁶, 68×10⁶, 69×10⁶, 70×10⁶, 71×10⁶, 72×10⁶, 73×10⁶, 74×10⁶, 75×10⁶, 76×10⁶, 77×10⁶, 78×10⁶, 79×10⁶, 80×10⁶, 81×10⁶, 82×10⁶, 83×10⁶, 84×10⁶, 85×10⁶, 86×10⁶, 87×10⁶, 88×10⁶, 89×10⁶, 90×10⁶, 91×10⁶, 92×10⁶, 93×10⁶, 94×10⁶, 95×10⁶, 96×10⁶, 97×10⁶, 98×10⁶, 99×10⁶, 100×10⁶, 101×10⁶, 102×10⁶, 103×10⁶, 104×10⁶, 105×10⁶, 106×10⁶, 107×10⁶, 108×10⁶, 109×10⁶, or 110×10⁶) viable CD22-specific CAR-T cells. In some embodiments, the dose is a therapeutically effective amount of viable CD22-specific CAR-T cells. In other embodiments, the dose is a clinically effective amount of viable CD22-specific CAR-T cells. In some embodiments, the viable CD22-specific CAR-T cells include CD22-specific CAR expressing CD4+ T cells and CD22-specific CAR expressing CD8+ T cells at a ratio of about 1:1.

In some embodiments, a subject is administered about 50×10⁶ to about 110×10⁶ (e.g., 50×10⁶, 51×10⁶, 52×10⁶, 53×10⁶, 54×10⁶, 55×10⁶, 56×10⁶, 57×10⁶, 58×10⁶, 59×10⁶, 60×10⁶, 61×10⁶, 62×10⁶, 63×10⁶, 64×10⁶, 65×10⁶, 66×10⁶, 67×10⁶, 68×10⁶, 69×10⁶, 70×10⁶, 71×10⁶, 72×10⁶, 73×10⁶, 74×10⁶, 75×10⁶, 76×10⁶, 77×10⁶, 78×10⁶, 79×10⁶, 80×10⁶, 81×10⁶, 82×10⁶, 83×10⁶, 84×10⁶, 85×10⁶, 86×10⁶, 87×10⁶, 88×10⁶, 89×10⁶, 90×10⁶, 91×10⁶, 92×10⁶, 93×10⁶, 94×10⁶, 95×10⁶, 96×10⁶, 97×10⁶, 98×10⁶, 99×10⁶, 100×10⁶, 101×10⁶, 102×10⁶, 103×10⁶, 104×10⁶, 105×10⁶, 106×10⁶, 107×10⁶, 108×10⁶, 109×10⁶, or 110×10⁶) viable CD22-specific CAR-T cells described herein. In some embodiments, the dose is a therapeutically effective amount of viable CD22-specific CAR-T cells. In other embodiments, the dose is a clinically effective amount of viable CD22-specific CAR-T cells. In some instances, 50% of the viable CD22-specific CAR-T cells are CD22-specific CAR expressing CD4+ T cells and 50% of the viable CD22-specific CAR-T cells are CD22-specific CAR expressing CD8+ T cells.

In some embodiments, the CD22-specific CAR-T cells described herein are administered to a subject at a dose of about 2×10⁶ per kg of body weight. In some embodiments, a maximum dose administered is about 2×10⁸ viable CD22-specific CAR-T cells. In some embodiments, the dose is a therapeutically effective amount of viable CD22-specific CAR-T cells. In other embodiments, the dose is a clinically effective amount of viable CD22-specific CAR-T cells.

In some embodiments, the CD22-specific CAR-T cells described herein are administered to a subject at a dose of up to about 2×10⁸ viable CD22-specific CAR-T cells. In some embodiments, a subject is administered from about 0.2×10⁶ to about 5.0×10⁶ (e.g., about 0.2×10⁶, 0.4×10⁶, 0.5×10⁶, 0.6×10⁶, 0.8×10⁶, 0.9×10⁶, 1.0×10⁶, 1.2×10⁶, 1.4×10⁶, 1.5×10⁶, 1.6×10⁶, 1.8×10⁶, 1.9×10⁶, 2.0×10⁶, 2.2×10⁶, 2.4×10⁶, 2.5×10⁶, 2.6×10⁶, 2.8×10⁶, 2.9×10⁶, 3.0×10⁶, 3.2×10⁶, 3.4×10⁶, 3.5×10⁶, 3.6×10⁶, 3.8×10⁶, 3.9×10⁶, 4.0×10⁶, 4.2×10⁶, 4.4×10⁶, 4.5×10⁶, 4.6×10⁶, 4.8×10⁶, 4.9×10⁶, or 5.0×10⁶) viable CD22-specific CAR-T cells per kg of body weight for a subject with a body weight of about 50 kg or less. In some embodiments, a subject is administered from about 0.1×10⁸ to about 2.5×10⁸ (e.g., about 0.1×10⁶, 0.2×10⁶, 0.4×10⁶, 0.5×10⁶, 0.6×10⁶, 0.8×10⁶, 0.9×10⁶, 1.0×10⁶, 1.2×10⁶, 1.4×10⁶, 1.5×10⁶, 1.6×10⁶, 1.8×10⁶, 1.9×10⁶, 2.0×10⁶, 2.2×10⁶, 2.4×10⁶, or 2.5×10⁶) viable CD22-specific CAR-T cells for a subject with a body weight of greater than about 50 kg. In some embodiments, a subject is administered from about 0.6×10⁸ to about 6.0×10⁸ (e.g., about 0.6×10⁸, 0.8×10⁸, 0.9×10⁸, 1.0×10⁸, 1.2×10⁸, 1.4×10⁸, 1.5×10⁸, 1.6×10⁸, 1.8×10⁸, 1.9×10⁸, 2.0×10⁸, 2.2×10⁸, 2.4×10⁸, 2.5×10⁸, 2.6×10⁸, 2.8×10⁸, 2.9×10⁸, 3.0×10⁸, 3.2×10⁸, 3.4×10⁸, 3.5×10⁸, 3.6×10⁸, 3.8×10⁸, 3.9×10⁸, 4.0×10⁸, 4.2×10⁸, 4.4×10⁸, 4.5×10⁸, 4.6×10⁸, 4.8×10⁸, 4.9×10⁸, 5.0×10⁸, 5.2×10⁸, 5.4×10⁸, 5.5×10⁸, 5.6×10⁸, 5.8×10⁸, 5.9×10⁸, or 6.0×10⁸) viable CD22-specific CAR-T cells. In some embodiments, the dose is a therapeutically effective amount of viable CD22-specific CAR-T cells. In other embodiments, the dose is a clinically effective amount of viable CD22-specific CAR-T cells.

In some embodiments, a single dose of any of the CD22-specific CAR-T cells described herein includes about 0.2×10⁶ to about 5.0×10⁶ (e.g., about 0.2×10⁶, 0.3×10⁶, 0.4×10⁶, 0.5×10⁶, 0.6×10⁶, 0.7×10⁶, 0.8×10⁶, 0.9×10⁶, 1.0×10⁶, 1.1×10⁶, 1.2×10⁶, 1.3×10⁶, 1.4×10⁶, 1.5×10⁶, 1.6×10⁶, 1.7×10⁶, 1.8×10⁶, 1.9×10⁶, 2.0×10⁶, 2.1×10⁶, 2.2×10⁶, 2.3×10⁶, 2.4×10⁶, 2.5×10⁶, 2.6×10⁶, 2.7×10⁶, 2.8×10⁶, 2.9×10⁶, 3.0×10⁶, 3.1×10⁶, 3.2×10⁶, 3.3×10⁶, 3.4×10⁶, 3.5×10⁶, 3.6×10⁶, 3.7×10⁶, 3.8×10⁶, 3.9×10⁶, 4.0×10⁶, 4.1×10⁶, 4.2×10⁶, 4.3×10⁶, 4.4×10⁶, 4.5×10⁶, 4.6×10⁶, 4.7×10⁶, 4.8×10⁶, 4.9×10⁶, or 5.0×10⁶) viable CD22-specific CAR-T cells per kg of body weight for a subject with a body weight of 50 kg or less. In some embodiments, a single dose of any of the CD22-specific CAR-T cells described herein includes about 0.1×10⁸ to about 2.5×10⁸ (e.g., about 0.1×10⁶, 0.2×10⁶, 0.3×10⁶, 0.4×10⁶, 0.5×10⁶, 0.6×10⁶, 0.7×10⁶, 0.8×10⁶, 0.9×10⁶, 1.0×10⁶, 1.1×10⁶, 1.2×10⁶, 1.3×10⁶, 1.4×10⁶, 1.5×10⁶, 1.6×10⁶, 1.7×10⁶, 1.8×10⁶, 1.9×10⁶, 2.0×10⁶, 2.1×10⁶, 2.2×10⁶, 2.3×10⁶, 2.4×10⁶, or 2.5×10⁶) viable CD22-specific CAR-T cells per kg of body weight for a subject with a body weight of more than 50 kg. In some embodiments, a single dose of any of the CD22-specific CAR-T cells described herein includes about 0.6×10⁸ to about 6.0×10⁸ (e.g., about 0.6×10⁸, 0.7×10⁸, 0.8×10⁸, 0.9×10⁸, 1.0×10⁸, 1.1×10⁸, 1.2×10⁸, 1.3×10⁸, 1.4×10⁸, 1.5×10⁸, 1.6×10⁸, 1.7×10⁸, 1.8×10⁸, 1.9×10⁸, 2.0×10⁸, 2.1×10⁸, 2.2×10⁸, 2.3×10⁸, 2.4×10⁸, 2.5×10⁸, 2.6×10⁸, 2.7×10⁸, 2.8×10⁸, 2.9×10⁸, 3.0×10⁸, 3.1×10⁸, 3.2×10⁸, 3.3×10⁸, 3.4×10⁸, 3.5×10⁸, 3.6×10⁸, 3.7×10⁸, 3.8×10⁸, 3.9×10⁸, 4.0×10⁸, 4.1×10⁸, 4.2×10⁸, 4.3×10⁸, 4.4×10⁸, 4.5×10⁸, 4.6×10⁸, 4.7×10⁸, 4.8×10⁸, 4.9×10⁸, 5.0×10⁸, 5.1×10⁸, 5.2×10⁸, 5.3×10⁸, 5.4×10⁸, 5.5×10⁸, 5.6×10⁸, 5.7×10⁸, 5.8×10⁸, 5.9×10⁸, or 6.0×10⁸) viable CD22-specific CAR-T cells. In some embodiments, a single infusion bag of any of the CD22-specific CAR-T cells described herein includes about 0.6×10⁸ to about 6.0×10⁸ (e.g., about 0.6×10⁸, 0.7×10⁸, 0.8×10⁸, 0.9×108, 1.0×10⁸, 1.1×10⁸, 1.2×10⁸, 1.3×10⁸, 1.4×10⁸, 1.5×10⁸, 1.6×10⁸, 1.7×10⁸, 1.8×10⁸, 1.9×10⁸, 2.0×10⁸, 2.1×10⁸, 2.2×10⁸, 2.3×10⁸, 2.4×10⁸, 2.5×10⁸, 2.6×10⁸, 2.7×10⁸, 2.8×10⁸, 2.9×10⁸, 3.0×10⁸, 3.1×10⁸, 3.2×10⁸, 3.3×10⁸, 3.4×10⁸, 3.5×10⁸, 3.6×10⁸, 3.7×10⁸, 3.8×10⁸, 3.9×10⁸, 4.0×10⁸, 4.1×10⁸, 4.2×10⁸, 4.3×10⁸, 4.4×10⁸, 4.5×10⁸, 4.6×10⁸, 4.7×10⁸, 4.8×10⁸, 4.9×10⁸, 5.0×10⁸, 5.1×10⁸, 5.2×10⁸, 5.3×10⁸, 5.4×10⁸, 5.5×10⁸, 5.6×10⁸, 5.7×10⁸, 5.8×10⁸, 5.9×10⁸, or 6.0×10⁸) viable CD22-specific CAR-T cells in a cell suspension of from about 10 mL to about 50 mL. In some embodiments, the dose is a therapeutically effective amount of viable CD22-specific CAR-T cells. In other embodiments, the dose is a clinically effective amount of viable CD22-specific CAR-T cells.

In some embodiments, the therapeutically effective dose or clinically effective dose comprises about the same number of CAR-T cells as were included in the prior CD19-CAR-T pharmaceutical composition. In some embodiments, the therapeutically effective dose or clinically effective dose comprises more or a greater number of CAR-T cells than were included in the prior CD19-CAR-T pharmaceutical composition. In some embodiments, the therapeutically effective dose or clinically effective dose comprises fewer or a lower number of CAR-T cells than were included in the prior CD19-CAR-T pharmaceutical composition, therapeutically effective dose or clinically effective dose.

### A. CD4+ CAR+ T Cells and CD8+ CAR+ T Cells

In some embodiments, a subject is administered viable CD4+ CAR+ T cells and viable CD8+ CAR+ T cells. In some embodiments, the viable CD4+ CAR+ T cells and viable CD8+ CAR+ T cells are administered at the same time or simultaneously.

In some embodiments, the viable CD4+ CAR+ T cells and viable CD8+ CAR+ T cells are administered sequentially. For instance, the viable CD4+ CAR+ T cells are administered before the viable CD8+ CAR+ T cells. In some embodiments, the viable CD4+ CAR+ T cells are administered after the viable CD8+ CAR+ T cells are administered. In some embodiments, the viable CD4+ CAR+ T cells are administered at least 1 hour, at least 2 hours, at least 3 hours, at least 4 hours at least 5 hours, at least 6 hours, at least 8 hours, at least 10 hours, at least 12 hours, at least 16 hours, at least 20 hours, at least 24 hours, at least 2 days, at least 3 days, at least 4 days, at least 5 days, at least 6 days, at least 7 days, at least 2 weeks, at least 3 weeks, at least 4 weeks, at least 2 months, at least 3 months, at least 4 months, at least 5 months, at least 6 months, or more, prior to the administration of the viable CD8+ CAR+ T cells.

In some embodiments, the subject is administered the CD4+ CAR+ T cells at least 10 minutes, at least 20 minutes, at least 30 minutes, at least 40 minutes, at least 50 minutes, at least 60 minutes, at least 70 minutes, at least 80 minutes, at least 90 minutes or more minutes before the administration of the CD8+ CAR+ T cells.

In some embodiments, the subject is administered the CD4+ CAR+ T cells at least 1 hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 13 hours, at least 14 hours, at least 15 hours, at least 16 hours, at least 17 hours, at least 18 hours, at least 19 hours, at least 20 hours, at least 21 hours, at least 22 hours, at least 23 hours, at least 24 hours, at least 25 hours, at least 26 hours, at least 27 hours, at least 28 hours, at least 29 hours, at least 30 hours, at least 31 hours, at least 32 hours, at least 33 hours, at least 34 hours, at least 35 hours, at least 36 hours, at least 48 hours, at least 72 hours or more hours before the administration of the CD8+ CAR+ T cells.

In some embodiments, the subject is administered the CD4+ CAR+ T cells at least 2 days, at least 3 days, at least 4 days, at least 5 days, at least 6 days, at least 7 days, at least 8 days, at least 9 days, at least 10 days, at least 11 days, at least 12 days, at least 13 days, at least 2 weeks, at least 3 weeks, at least 4 weeks, at least 2 months, at least 3 months, at least 4 months, at least 5 months, at least 6 months, at least 7 months, at least 8 months, at least 9 months, at least 10 months, at least 11 months or more before the administration of the CD8+ CAR+ T cells.

In some embodiments, the viable CD8+ CAR+ T cells are administered at least 1 hour, at least 2 hours, at least 3 hours, at least 4 hours at least 5 hours, at least 6 hours, at least 8 hours, at least 10 hours, at least 12 hours, at least 16 hours, at least 20 hours, at least 24 hours, at least 2 days, at least 3 days, at least 4 days, at least 5 days, at least 6 days, at least 7 days, at least 2 weeks, at least 3 weeks, at least 4 weeks, at least 2 months, at least 3 months, at least 4 months, at least 5 months, at least 6 months, or more, prior to the administration of the viable CD4+ CAR+ T cells.

In some embodiments, the subject is administered the CD8+ CAR+ T cells at least 10 minutes, at least 20 minutes, at least 30 minutes, at least 40 minutes, at least 50 minutes, at least 60 minutes, at least 70 minutes, at least 80 minutes, at least 90 minutes or more minutes before the administration of the CD4+ CAR+ T cells.

In some embodiments, the subject is administered the CD8+ CAR+ T cells at least 1 hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 13 hours, at least 14 hours, at least 15 hours, at least 16 hours, at least 17 hours, at least 18 hours, at least 19 hours, at least 20 hours, at least 21 hours, at least 22 hours, at least 23 hours, at least 24 hours, at least 25 hours, at least 26 hours, at least 27 hours, at least 28 hours, at least 29 hours, at least 30 hours, at least 31 hours, at least 32 hours, at least 33 hours, at least 34 hours, at least 35 hours, at least 36 hours, at least 48 hours, at least 72 hours or more hours before the administration of the CD4+ CAR+ T cells.

In some embodiments, the subject is administered the CD8+ CAR+ T cells at least 2 days, at least 3 days, at least 4 days, at least 5 days, at least 6 days, at least 7 days, at least 8 days, at least 9 days, at least 10 days, at least 11 days, at least 12 days, at least 13 days, at least 2 weeks, at least 3 weeks, at least 4 weeks, at least 2 months, at least 3 months, at least 4 months, at least 5 months, at least 6 months, at least 7 months, at least 8 months, at least 9 months, at least 10 months, at least 11 months or more before the administration of the CD4+ CAR+ T cells.

In some embodiments, the dosing regimen of CD4+ CAR+ T cells and viable CD8+ CAR+ T cells is the same as was used in the prior CD19-CAR-T therapy. In some embodiments, the dosing regimen of CD4+ CAR+ T cells and viable CD8+ CAR+ T cells is different from what was used in the prior CD19-CAR-T therapy.

In some embodiments, the subject is administered a ratio of CD4+ CAR+ T cells to CD8+ CAR+ T cells such that the ratio is selected from the group consisting of 0.25:1, 0.5:1, 0.75:1, 1:1, 1.5:1, 2:1, 3:1, 4:1, and 5:1.

In some embodiments, the ratio of CD4+ CAR+ T cells to CD8+ CAR+ T cells is the same as was used in the prior CD19-CAR-T therapy. In some embodiments, the ratio of CD4+ CAR+ T cells to CD8+ CAR+ T cells is different from what was used in the prior CD19-CAR-T therapy. In some embodiments, the ratio of CD4+ CAR+ T cells to CD8+ CAR+ T cells is greater than what was used in the prior CD19-CAR-T therapy. In some embodiments, the ratio of CD4+ CAR+ T cells to CD8+ CAR+ T cells is less than what was used in the prior CD19-CAR-T therapy.

In some embodiments, the CD4+ CAR+ T cells are selected from the group consisting of a population of autologous CD4+ CAR+ T cells, a population of allogeneic CD4+ CAR+ T cells, and a combination thereof. In some embodiments, the CD8+ CAR+ T cells are selected from the group consisting of a population of autologous CD8+ CAR+ T cells, a population of allogeneic CD8+ CAR+ T cells, and a combination thereof. In some embodiments, the bulk population of CAR+ T cells are selected from the group consisting of a population of autologous CAR+ T cells, a population of allogeneic CAR+ T cells, and a combination thereof. b. CD4+ CAR+ T cells and Bulk Population of CAR+ T cells

In some embodiments, a subject is administered viable CD4+ CAR+ T cells and a viable bulk population of CAR+ T cells. In some embodiments, the viable CD4+ CAR+ T cells and the viable bulk CAR+ T cells are administered at the same time or simultaneously.

In some embodiments, the viable CD4+ CAR+ T cells and viable bulk CAR+ T cells are administered sequentially. For instance, the viable CD4+ CAR+ T cells are administered before the bulk CAR+ T cells. In some embodiments, the viable CD4+ CAR+ T cells are administered after the bulk CAR+ T cells are administered. In some embodiments, the viable CD4+ CAR+ T cells are administered at least 1 hour, at least 2 hours, at least 3 hours, at least 4 hours at least 5 hours, at least 6 hours, at least 8 hours, at least 10 hours, at least 12 hours, at least 16 hours, at least 20 hours, at least 24 hours, at least 2 days, at least 3 days, at least 4 days, at least 5 days, at least 6 days, at least 7 days, at least 2 weeks, at least 3 weeks, at least 4 weeks, at least 2 months, at least 3 months, at least 4 months, at least 5 months, at least 6 months, or more, prior to the administration of the viable bulk CAR+ T cells.

In some embodiments, the subject is administered the CD4+ CAR+ T cells at least 10 minutes, at least 20 minutes, at least 30 minutes, at least 40 minutes, at least 50 minutes, at least 60 minutes, at least 70 minutes, at least 80 minutes, at least 90 minutes or more minutes before the administration of the bulk CAR+ T cells.

In some embodiments, the subject is administered the CD4+ CAR+ T cells at least 1 hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 13 hours, at least 14 hours, at least 15 hours, at least 16 hours, at least 17 hours, at least 18 hours, at least 19 hours, at least 20 hours, at least 21 hours, at least 22 hours, at least 23 hours, at least 24 hours, at least 25 hours, at least 26 hours, at least 27 hours, at least 28 hours, at least 29 hours, at least 30 hours, at least 31 hours, at least 32 hours, at least 33 hours, at least 34 hours, at least 35 hours, at least 36 hours, at least 48 hours, at least 72 hours or more hours before the administration of the bulk CAR+ T cells.

In some embodiments, the subject is administered the CD4+ CAR+ T cells at least 2 days, at least 3 days, at least 4 days, at least 5 days, at least 6 days, at least 7 days, at least 8 days, at least 9 days, at least 10 days, at least 11 days, at least 12 days, at least 13 days, at least 2 weeks, at least 3 weeks, at least 4 weeks, at least 2 months, at least 3 months, at least 4 months, at least 5 months, at least 6 months, at least 7 months, at least 8 months, at least 9 months, at least 10 months, at least 11 months or more before the administration of the bulk CAR+ T cells.

In some embodiments, the viable bulk CAR+ T cells are administered at least 1 hour, at least 2 hours, at least 3 hours, at least 4 hours at least 5 hours, at least 6 hours, at least 8 hours, at least 10 hours, at least 12 hours, at least 16 hours, at least 20 hours, at least 24 hours, at least 2 days, at least 3 days, at least 4 days, at least 5 days, at least 6 days, at least 7 days, at least 2 weeks, at least 3 weeks, at least 4 weeks, at least 2 months, at least 3 months, at least 4 months, at least 5 months, at least 6 months, or more, prior to the administration of the viable CD4+ CAR+ T cells.

In some embodiments, the subject is administered the bulk CAR+ T cells at least 10 minutes, at least 20 minutes, at least 30 minutes, at least 40 minutes, at least 50 minutes, at least 60 minutes, at least 70 minutes, at least 80 minutes, at least 90 minutes or more minutes before the administration of the CD4+ CAR+ T cells.

In some embodiments, the subject is administered the bulk CAR+ T cells at least 1 hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 13 hours, at least 14 hours, at least 15 hours, at least 16 hours, at least 17 hours, at least 18 hours, at least 19 hours, at least 20 hours, at least 21 hours, at least 22 hours, at least 23 hours, at least 24 hours, at least 25 hours, at least 26 hours, at least 27 hours, at least 28 hours, at least 29 hours, at least 30 hours, at least 31 hours, at least 32 hours, at least 33 hours, at least 34 hours, at least 35 hours, at least 36 hours, at least 48 hours, at least 72 hours or more hours before the administration of the CD4+ CAR+ T cells.

In some embodiments, the subject is administered the bulk CAR+ T cells at least 2 days, at least 3 days, at least 4 days, at least 5 days, at least 6 days, at least 7 days, at least 8 days, at least 9 days, at least 10 days, at least 11 days, at least 12 days, at least 13 days, at least 2 weeks, at least 3 weeks, at least 4 weeks, at least 2 months, at least 3 months, at least 4 months, at least 5 months, at least 6 months, at least 7 months, at least 8 months, at least 9 months, at least 10 months, at least 11 months or more before the administration of the CD4+ CAR+ T cells.

In some embodiments, the dosing regimen of CD4+ CAR+ T cells and bulk CAR+ T cells is the same as was used in the prior CD19-CAR-T therapy. In some embodiments, the dosing regimen of CD4+ CAR+ T cells and bulk CAR+ T cells is different from what was used in the prior CD19-CAR-T therapy.

In some embodiments, the subject is administered a ratio of CD4+ CAR+ T cells to bulk CAR+ T cells such that the ratio is selected from the group consisting of 0.25:1, 0.5:1, 0.75:1, 1:1, 1.5:1, 2:1, 3:1, 4:1, and 5:1.

In some embodiments, the ratio of CD4+ CAR+ T cells to bulk CAR+ T cells is the same as was used in the prior CD19-CAR-T therapy. In some embodiments, the ratio of CD4+ CAR+ T cells to bulk CAR+ T cells is different from what was used in the prior CD19-CAR-T therapy. In some embodiments, the ratio of CD4+ CAR+ T cells to bulk CAR+ T cells is greater than what was used in the prior CD19-CAR-T therapy. In some embodiments, the ratio of CD4+ CAR+ T cells to bulk CAR+ T cells is less than what was used in the prior CD19-CAR-T therapy.

In some embodiments, the CD4+ CAR+ T cells are selected from the group consisting of a population of autologous CD4+ CAR+ T cells, a population of allogeneic CD4+ CAR+ T cells, and a combination thereof. In some embodiments, the bulk CAR+ T cells are selected from the group consisting of a population of autologous bulk CAR+ T cells, a population of allogeneic bulk CAR+ T cells, and a combination thereof. In some embodiments, the bulk population of CAR+ T cells are selected from the group consisting of a population of autologous CAR+ T cells, a population of allogeneic CAR+ T cells, and a combination thereof.

### C. Bulk Population of CAR+ T Cells and CD8+ CAR+ T Cells

In some embodiments, a subject is administered a viable bulk population of CAR+ T cells and viable CD8+ CAR+ T cells. In some embodiments, the viable bulk CAR+ T cells and viable CD8+ CAR+ T cells are administered at the same time or simultaneously.

In some embodiments, the viable bulk CAR+ T cells and viable CD8+ CAR+ T cells are administered sequentially. For instance, the viable bulk CAR+ T cells are administered before the viable CD8+ CAR+ T cells. In some embodiments, the viable bulk CAR+ T cells are administered after the viable CD8+ CAR+ T cells are administered. In some embodiments, the viable bulk CAR+ T cells are administered at least 1 hour, at least 2 hours, at least 3 hours, at least 4 hours at least 5 hours, at least 6 hours, at least 8 hours, at least 10 hours, at least 12 hours, at least 16 hours, at least 20 hours, at least 24 hours, at least 2 days, at least 3 days, at least 4 days, at least 5 days, at least 6 days, at least 7 days, at least 2 weeks, at least 3 weeks, at least 4 weeks, at least 2 months, at least 3 months, at least 4 months, at least 5 months, at least 6 months, or more, prior to the administration of the viable CD8+ CAR+ T cells.

In some embodiments, the subject is administered the bulk CAR+ T cells at least 10 minutes, at least 20 minutes, at least 30 minutes, at least 40 minutes, at least 50 minutes, at least 60 minutes, at least 70 minutes, at least 80 minutes, at least 90 minutes or more minutes before the administration of the CD8+ CAR+ T cells.

In some embodiments, the subject is administered the bulk CAR+ T cells at least 1 hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 13 hours, at least 14 hours, at least 15 hours, at least 16 hours, at least 17 hours, at least 18 hours, at least 19 hours, at least 20 hours, at least 21 hours, at least 22 hours, at least 23 hours, at least 24 hours, at least 25 hours, at least 26 hours, at least 27 hours, at least 28 hours, at least 29 hours, at least 30 hours, at least 31 hours, at least 32 hours, at least 33 hours, at least 34 hours, at least 35 hours, at least 36 hours, at least 48 hours, at least 72 hours or more hours before the administration of the CD8+ CAR+ T cells.

In some embodiments, the subject is administered the bulk CAR+ T cells at least 2 days, at least 3 days, at least 4 days, at least 5 days, at least 6 days, at least 7 days, at least 8 days, at least 9 days, at least 10 days, at least 11 days, at least 12 days, at least 13 days, at least 2 weeks, at least 3 weeks, at least 4 weeks, at least 2 months, at least 3 months, at least 4 months, at least 5 months, at least 6 months, at least 7 months, at least 8 months, at least 9 months, at least 10 months, at least 11 months or more before the administration of the CD8+ CAR+ T cells.

In some embodiments, the viable CD8+ CAR+ T cells are administered at least 1 hour, at least 2 hours, at least 3 hours, at least 4 hours at least 5 hours, at least 6 hours, at least 8 hours, at least 10 hours, at least 12 hours, at least 16 hours, at least 20 hours, at least 24 hours, at least 2 days, at least 3 days, at least 4 days, at least 5 days, at least 6 days, at least 7 days, at least 2 weeks, at least 3 weeks, at least 4 weeks, at least 2 months, at least 3 months, at least 4 months, at least 5 months, at least 6 months, or more, prior to the administration of the viable bulk CAR+ T cells.

In some embodiments, the subject is administered the CD8+ CAR+ T cells at least 10 minutes, at least 20 minutes, at least 30 minutes, at least 40 minutes, at least 50 minutes, at least 60 minutes, at least 70 minutes, at least 80 minutes, at least 90 minutes or more minutes before the administration of the bulk CAR+ T cells.

In some embodiments, the subject is administered the CD8+ CAR+ T cells at least 1 hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 13 hours, at least 14 hours, at least 15 hours, at least 16 hours, at least 17 hours, at least 18 hours, at least 19 hours, at least 20 hours, at least 21 hours, at least 22 hours, at least 23 hours, at least 24 hours, at least 25 hours, at least 26 hours, at least 27 hours, at least 28 hours, at least 29 hours, at least 30 hours, at least 31 hours, at least 32 hours, at least 33 hours, at least 34 hours, at least 35 hours, at least 36 hours, at least 48 hours, at least 72 hours or more hours before the administration of the bulk CAR+ T cells.

In some embodiments, the subject is administered the CD8+ CAR+ T cells at least 2 days, at least 3 days, at least 4 days, at least 5 days, at least 6 days, at least 7 days, at least 8 days, at least 9 days, at least 10 days, at least 11 days, at least 12 days, at least 13 days, at least 2 weeks, at least 3 weeks, at least 4 weeks, at least 2 months, at least 3 months, at least 4 months, at least 5 months, at least 6 months, at least 7 months, at least 8 months, at least 9 months, at least 10 months, at least 11 months or more before the administration of the bulk CAR+ T cells.

In some embodiments, the dosing regimen of bulk CAR+ T cells and viable CD8+ CAR+ T cells is the same as was used in the prior CD19-CAR-T therapy. In some embodiments, the dosing regimen of bulk CAR+ T cells and viable CD8+ CAR+ T cells is different from what was used in the prior CD19-CAR-T therapy.

In some embodiments, the subject is administered a ratio of bulk CAR+ T cells to CD8+ CAR+ T cells such that the ratio is selected from the group consisting of 0.25:1, 0.5:1, 0.75:1, 1:1, 1.5:1, 2:1, 3:1, 4:1, and 5:1.

In some embodiments, the ratio of bulk CAR+ T cells to CD8+ CAR+ T cells is the same as was used in the prior CD19-CAR-T therapy. In some embodiments, the ratio of bulk CAR+ T cells to CD8+ CAR+ T cells is different from what was used in the prior CD19-CAR-T therapy. In some embodiments, the ratio of bulk CAR+ T cells to CD8+ CAR+ T cells is greater than what was used in the prior CD19-CAR-T therapy. In some embodiments, the ratio of bulk CAR+ T cells to CD8+ CAR+ T cells is less than what was used in the prior CD19-CAR-T therapy.

In some embodiments, the bulk CAR+ T cells are selected from the group consisting of a population of autologous bulk CAR+ T cells, a population of allogeneic bulk CAR+ T cells, and a combination thereof. In some embodiments, the CD8+ CAR+ T cells are selected from the group consisting of a population of autologous CD8+ CAR+ T cells, a population of allogeneic CD8+ CAR+ T cells, and a combination thereof. In some embodiments, the bulk population of CAR+ T cells are selected from the group consisting of a population of autologous CAR+ T cells, a population of allogeneic CAR+ T cells, and a combination thereof.

### D. CD22-CAR-T Cells and CD19-CAR-T Cells

In some embodiments, a subject is administered viable CD22-CAR-T cells and viable CD19-CAR-T cells. In some embodiments, the viable CD22-CAR-T cells and viable CD19-CAR-T cells are administered at the same time or simultaneously.

In some embodiments, the viable CD22-CAR-T cells and viable CD19-CAR-T cells are administered sequentially. For instance, the viable CD22-CAR-T cells are administered before the viable CD19-CAR-T cells. In some embodiments, the viable CD22-CAR-T cells are administered after the viable CD19-CAR-T cells are administered. In some embodiments, the viable CD22-CAR-T cells are administered at least 1 hour, at least 2 hours, at least 3 hours, at least 4 hours at least 5 hours, at least 6 hours, at least 8 hours, at least 10 hours, at least 12 hours, at least 16 hours, at least 20 hours, at least 24 hours, at least 2 days, at least 3 days, at least 4 days, at least 5 days, at least 6 days, at least 7 days, at least 2 weeks, at least 3 weeks, at least 4 weeks, at least 2 months, at least 3 months, at least 4 months, at least 5 months, at least 6 months, or more, prior to the administration of the viable CD19-CAR-T cells.

In some embodiments, the subject is administered the CD22-CAR-T cells at least 10 minutes, at least 20 minutes, at least 30 minutes, at least 40 minutes, at least 50 minutes, at least 60 minutes, at least 70 minutes, at least 80 minutes, at least 90 minutes or more minutes before the administration of the CD19-CAR-T cells.

In some embodiments, the subject is administered the CD22-CAR-T cells at least 1 hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 13 hours, at least 14 hours, at least 15 hours, at least 16 hours, at least 17 hours, at least 18 hours, at least 19 hours, at least 20 hours, at least 21 hours, at least 22 hours, at least 23 hours, at least 24 hours, at least 25 hours, at least 26 hours, at least 27 hours, at least 28 hours, at least 29 hours, at least 30 hours, at least 31 hours, at least 32 hours, at least 33 hours, at least 34 hours, at least 35 hours, at least 36 hours, at least 48 hours, at least 72 hours or more hours before the administration of the CD19-CAR-T cells.

In some embodiments, the subject is administered the CD22-CAR-T cells at least 2 days, at least 3 days, at least 4 days, at least 5 days, at least 6 days, at least 7 days, at least 8 days, at least 9 days, at least 10 days, at least 11 days, at least 12 days, at least 13 days, at least 2 weeks, at least 3 weeks, at least 4 weeks, at least 2 months, at least 3 months, at least 4 months, at least 5 months, at least 6 months, at least 7 months, at least 8 months, at least 9 months, at least 10 months, at least 11 months or more before the administration of the CD19-CAR-T cells.

In some embodiments, the viable CD19-CAR-T cells are administered at least 1 hour, at least 2 hours, at least 3 hours, at least 4 hours at least 5 hours, at least 6 hours, at least 8 hours, at least 10 hours, at least 12 hours, at least 16 hours, at least 20 hours, at least 24 hours, at least 2 days, at least 3 days, at least 4 days, at least 5 days, at least 6 days, at least 7 days, at least 2 weeks, at least 3 weeks, at least 4 weeks, at least 2 months, at least 3 months, at least 4 months, at least 5 months, at least 6 months, or more, prior to the administration of the viable CD22-CAR-T cells.

In some embodiments, the subject is administered the CD19-CAR-T cells at least 10 minutes, at least 20 minutes, at least 30 minutes, at least 40 minutes, at least 50 minutes, at least 60 minutes, at least 70 minutes, at least 80 minutes, at least 90 minutes or more minutes before the administration of the CD22-CAR-T cells.

In some embodiments, the subject is administered the CD19-CAR-T cells at least 1 hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 13 hours, at least 14 hours, at least 15 hours, at least 16 hours, at least 17 hours, at least 18 hours, at least 19 hours, at least 20 hours, at least 21 hours, at least 22 hours, at least 23 hours, at least 24 hours, at least 25 hours, at least 26 hours, at least 27 hours, at least 28 hours, at least 29 hours, at least 30 hours, at least 31 hours, at least 32 hours, at least 33 hours, at least 34 hours, at least 35 hours, at least 36 hours, at least 48 hours, at least 72 hours or more hours before the administration of the CD22-CAR-T cells.

In some embodiments, the subject is administered the CD19-CAR-T cells at least 2 days, at least 3 days, at least 4 days, at least 5 days, at least 6 days, at least 7 days, at least 8 days, at least 9 days, at least 10 days, at least 11 days, at least 12 days, at least 13 days, at least 2 weeks, at least 3 weeks, at least 4 weeks, at least 2 months, at least 3 months, at least 4 months, at least 5 months, at least 6 months, at least 7 months, at least 8 months, at least 9 months, at least 10 months, at least 11 months or more before the administration of the CD22-CAR-T cells.

In some embodiments, the dosing regimen of CD22-CAR-T cells and viable CD19-CAR-T cells is the same as was used in the prior CD19-CAR-T therapy. In some embodiments, the dosing regimen of CD22-CAR-T cells and viable CD19-CAR-T cells is different from what was used in the prior CD19-CAR-T therapy.

In some embodiments, the subject is administered a ratio of CD22-CAR-T cells to CD19-CAR-T cells such that the ratio is selected from the group consisting of 0.25:1, 0.5:1, 0.75:1, 1:1, 1.5:1, 2:1, 3:1, 4:1, and 5:1.

In some embodiments, the ratio of CD22-CAR-T cells to CD19-CAR-T cells is the same as was used in the prior CD19-CAR-T therapy. In some embodiments, the ratio of CD22-CAR-T cells to CD19-CAR-T cells is different from what was used in the prior CD19-CAR-T therapy. In some embodiments, the ratio of CD22-CAR-T cells to CD19-CAR-T cells is greater than what was used in the prior CD19-CAR-T therapy. In some embodiments, the ratio of CD22-CAR-T cells to CD19-CAR-T cells is less than what was used in the prior CD19-CAR-T therapy.

In some embodiments, the CD22-CAR-T cells are selected from the group consisting of a population of autologous CD22-CAR-T cells, a population of allogeneic CD22-CAR-T cells, and a combination thereof. In some embodiments, the CD19-CAR-T cells are selected from the group consisting of a population of autologous CD19-CAR-T cells, a population of allogeneic CD19-CAR-T cells, and a combination thereof.

### 3. Immunosuppressive Agents

In some embodiments, an immunosuppressive and/or immunomodulatory agent is not administered to the patient before the first administration of the population of engineered CAR-T cells. In certain embodiments, an immunosuppressive and/or immunomodulatory agent is administered to the patient before the first administration of the population of engineered CAR-T cells. In some embodiments, a standard or a light immunosuppressive regimen is administered to the patient before the first administration of the population of engineered CAR-T cells. In some embodiments, a heavy immunosuppressive regimen is administered to the patient before the first administration of the population of engineered CAR-T cells.

In some embodiments, an immunosuppressive and/or immunomodulatory agent is not administered to the patient before the prior therapy. In certain embodiments, an immunosuppressive and/or immunomodulatory agent is administered to the patient before the prior therapy. In some embodiments, a standard or a light immunosuppressive regimen is administered to the patient before the prior therapy. In some embodiments, a heavy immunosuppressive regimen is administered to the patient before the prior therapy.

In some embodiments, a heavy immunosuppressive regimen is administered to the patient before the prior therapy, and a standard or light immunosuppressive regimen is administered to the patient before the first administration of the population of engineered CAR-T cells. In some embodiments, a standard or light immunosuppressive regimen is administered to the patient before the prior therapy, and a standard or light immunosuppressive regimen is administered to the patient before the first administration of the population of engineered CAR-T cells. In some embodiments, a standard or light immunosuppressive regimen is administered to the patient before the prior therapy, and a heavy immunosuppressive regimen is administered to the patient before the first administration of the population of engineered CAR-T cells.

In some embodiments, a standard or a light immunosuppressive regimen comprises cyclophosphamide at about 500 mg/m2 and fludarabine at about 30 mg/m2, every day (q.d.) for 3 days. In some embodiments, a heavy immunosuppressive regimen comprises cyclophosphamide at about 500 mg/m2, or higher, and fludarabine at about 30 mg/m2, every day (q.d.) for 5 days. In some embodiments, a heavy immunosuppressive regimen comprises cyclophosphamide at about 500 mg/m2, or higher, and fludarabine at about 30 mg/m2, every day (q.d.) for 5 days, with alumtuzumab. In some embodiments, a heavy immunosuppressive regimen comprises cyclophosphamide at about 500 mg/m2, or lower, and fludarabine at about 30 mg/m2, every day (q.d.) for 5 days. In some embodiments, a heavy immunosuppressive regimen comprises cyclophosphamide at about 500 mg/m2, or lower, and fludarabine at about 30 mg/m2, every day (q.d.) for 5 days, with alumtuzumab.

In some embodiments, an immunosuppressive and/or immunomodulatory agent is administered at least 1, 2, 3, 4, 5, 6, 7, 8, 9, 10, 11, 12, 13, 14 days or more before the prior therapy and/or before the first administration of the engineered CAR-T cells. In some embodiments, an immunosuppressive and/or immunomodulatory agent is administered at least 1 week, 2 weeks, 3 weeks, 4 weeks, 5 weeks, 6 weeks, 7 weeks, 8 weeks, 9 weeks, 10 weeks or more before the prior therapy and/or before the first administration of the engineered CAR-T cells. In particular embodiments, an immunosuppressive and/or immunomodulatory agent is not administered to the patient after the prior therapy and/or after the first administration of the engineered CAR-T cells, or is administered at least 1, 2, 3, 4, 5, 6, 7, 8, 9, 10, 11, 12, 13, 14 days or more after the prior therapy and/or after the first administration of the engineered CAR-T cells. In some embodiments, an immunosuppressive and/or immunomodulatory agent is administered at least 1 week, 2 weeks, 3 weeks, 4 weeks, 5 weeks, 6 weeks, 7 weeks, 8 weeks, 9 weeks, 10 weeks or more after the prior therapy and/or after the first administration of the engineered CAR-T cells. Non-limiting examples of an immunosuppressive and/or immunomodulatory agent include cyclosporine, azathioprine, mycophenolic acid, mycophenolate mofetil, corticosteroids such as prednisone, methotrexate, gold salts, sulfasalazine, antimalarials, brequinar, leflunomide, mizoribine, 15-deoxyspergualine, 6-mercaptopurine, fludarabine, cyclophosphamide, rapamycin, tacrolimus (FK-506), OKT3, anti-thymocyte globulin, thymopentin, thymosin-α and similar agents. In some embodiments, the immunosuppressive and/or immunomodulatory agent is selected from a group of immunosuppressive antibodies consisting of antibodies binding to p75 of the IL-2 receptor, antibodies binding to, for instance, MHC, CD2, CD3, CD4, CD7, CD28, B7, CD40, CD45, IFN-gamma, TNF-.alpha., IL-4, IL-5, IL-6R, IL-6, IGF, IGFR1, IL-7, IL-8, IL-10, CD11a, or CD58, and antibodies binding to any of their ligands. In some embodiments where an immunosuppressive and/or immunomodulatory agent is administered to the patient before or after the prior therapy and/or the first administration of the engineered CAR-T cells, the administration is at a lower dosage than would be required for cells with MHC I and/or MHC II expression and without exogenous expression of CD47.

In one embodiment, such an immunosuppressive and/or immunomodulatory agent may be selected from soluble IL-15R, IL-10, B7 molecules (e.g., B7-1, B7-2, variants thereof, and fragments thereof), ICOS, and OX40, an inhibitor of a negative T cell regulator (such as an antibody against CTLA-4) and similar agents.

In some embodiments, an immunosuppressive and/or immunomodulatory agent is not administered to the patient before the prior therapy and/or before the administration of the engineered CAR-T cells. In certain embodiments, an immunosuppressive and/or immunomodulatory agent is administered to the patient before the prior therapy and/or before the first and/or second administration of the engineered CAR-T cells. In some embodiments, an immunosuppressive and/or immunomodulatory agent is administered at least 1, 2, 3, 4, 5, 6, 7, 8, 9, 10, 11, 12, 13, 14 days or more before the prior therapy and/or before the administration of the engineered CAR-T cells. In some embodiments, an immunosuppressive and/or immunomodulatory agent is administered at least 1 week, 2 weeks, 3 weeks, 4 weeks, 5 weeks, 6 weeks, 7 weeks, 8 weeks, 9 weeks, 10 weeks or more before the prior therapy and/or before the first and/or second administration of the engineered CAR-T cells. In particular embodiments, an immunosuppressive and/or immunomodulatory agent is administered at least 1, 2, 3, 4, 5, 6, 7, 8, 9, 10, 11, 12, 13, 14 days or more after the prior therapy and/or after the administration of the engineered CAR-T cells. In some embodiments, an immunosuppressive and/or immunomodulatory agent is administered at least 1 week, 2 weeks, 3 weeks, 4 weeks, 5 weeks, 6 weeks, 7 weeks, 8 weeks, 9 weeks, 10 weeks or more after the prior therapy and/or after the first and/or second administration of the engineered CAR-T cells. In some embodiments where an immunosuppressive and/or immunomodulatory agent is administered to the patient before or after the administration of the cells, the administration is at a lower dosage than would be required for cells with MHC I and/or MHC II expression and without exogenous expression of CD47.

In some embodiments, the immunodepleting therapy comprises administration of fludarabine and/or cyclophosphamide. In some embodiments, the immunodepleting therapy comprises IV infusion of about 1-100 mg/m² of fludarabine, about 1-50, about 10-50, about 20-50, about 30-50, about 40-50, about 50-100, about 60-100, about 70-100, about 80-100, about 90-100 mg/m² of fludarabine for about 1-7 days, about 2-7, about 3-7, about 4-7, about 5-7, about 6-7 days. In some embodiments, the immunodepleting therapy comprises IV infusion of about 1, about 5, about 10, about 20, about 30, about 40, about 50, about 60, about 70, about 80, about 90, or about 100 mg/m² of fludarabine for about 1, about 2, about 3, about 4, about 5, about 6, or about 7 days. In some embodiments, the immunodepleting therapy comprises IV infusion of about 100-1000 mg/m² of cyclophosphamide, about 100-500, about 200-500, about 300-500, about 400-500, about 500-1000, about 600-1000, about 700-1000, about 800-1000, about 900-1000 mg/m² of cyclophosphamide for about 1-7 days, about 2-7, about 3-7, about 4-7, about 5-7, about 6-7 days. In some embodiments, the immunodepleting therapy comprises IV infusion of about 100, about 200, about 300, about 400, about 500, about 600, about 700, about 800, about 900, about 1000 mg/m² of cyclophosphamide for about 1, about 2, about 3, about 4, about 5, about 6, or about 7 days.

In some embodiments, the immunodepleting therapy further comprises IV infusion of about 1 mg, about 2 mg, about 3 mg, about 4 mg, about 5 mg, about 6 mg, about 7 mg, about 8 mg, about 9 mg, about 10 mg, about 12 mg, about 14 mg, about 16 mg, about 18 mg, about 20 mg, about 22 mg, about 24 mg, about 26 mg, about 28 mg, or about 30 mg of alemtuzumab for about 1, about 2, about 3, about 4, about 5, about 6, or about 7 days.

All headings and section designations are used for clarity and reference purposes only and are not to be considered limiting in any way. For example, those of skill in the art will appreciate the usefulness of combining various embodiments from different headings and sections as appropriate according to the spirit and scope of the technology described herein.

All references cited herein are hereby incorporated by reference herein in their entireties and for all purposes to the same extent as if each individual publication or patent or patent application was specifically and individually indicated to be incorporated by reference in its entirety for all purposes.

Many modifications and variations of this application can be made without departing from its spirit and scope, as will be apparent to those skilled in the art. The specific embodiments and examples described herein are offered by way of example only, and the application is to be limited only by the terms of the appended claims, along with the full scope of equivalents to which the claims are entitled.

### Exemplary Embodiments

Embodiment 1. A method of treating a disease or disorder in a patient who has undergone one or more prior treatments for the disease or disorder, comprising evaluating the patient for the disease or disorder, and administering a population of engineered CAR-T cells to the patient to treat the disease or disorder, wherein the engineered CAR-T cells comprise an exogenous polynucleotide encoding one or more chimeric antigen receptors (CARs), wherein at least one CAR comprises a CD22 antigen binding domain having the CDR sequences from SEQ ID NO: 45, 54, 85, 91, 92, or 93.

Embodiment 2. A method of treating a disease or disorder characterized by antigen evasion in a patient who has undergone one or more prior treatments for the disease or disorder prior to antigen evasion, comprising evaluating the patient for the disease or disorder characterized by antigen evasion, and administering a population of engineered CAR-T cells to the patient to treat the disease or disorder characterized by antigen evasion, wherein the engineered CAR-T cells comprise an exogenous polynucleotide encoding one or more CARs, wherein at least one CAR comprises a CD22 antigen binding domain having the CDR sequences from SEQ ID NO: 45, 54, 85, 91, 92, or 93.

Embodiment 3. A method of treating a cancer characterized by antigen evasion in a patient who has undergone one or more prior treatments for the cancer prior to antigen evasion, comprising evaluating the patient for the disease or disorder characterized by antigen evasion, and administering a population of engineered CAR-T cells to the patient to treat the disease or disorder characterized by antigen evasion, wherein the engineered CAR-T cells comprise an exogenous polynucleotide encoding one or more CARs, wherein at least one CAR comprises a CD22 antigen binding domain having the CDR sequences from SEQ ID NO: 45, 54, 85, 91, 92, or 93.

Embodiment 4. A method of treating a disease or disorder in a patient who has undergone one or more prior treatments for the disease or disorder, comprising evaluating the patient for the disease or disorder, and administering a population of engineered CAR-T cells to the patient to treat the disease or disorder, wherein the engineered CAR-T cells comprise reduced expression of one or more major histocompatibility complex (MHC) class I and/or class II human leukocyte antigens (HLAs), and reduced expression of a T cell receptor (TCR) relative to an unaltered control cell, and a first exogenous polynucleotide encoding one or more CARs, wherein at least one CAR comprises a CD22 antigen binding domain having the CDR sequences from SEQ ID NO: 45, 54, 85, 91, 92, or 93.

Embodiment 5. A method of treating a disease or disorder in a patient who has undergone one or more prior treatments for the disease or disorder, comprising evaluating the patient for the disease or disorder, and administering a population of engineered CAR-T cells to the patient to treat the disease or disorder, wherein the engineered CAR-T cells comprise reduced expression of one or more MHC class I and/or class II HLA, and reduced expression of a TCR relative to an unaltered control cell, a first exogenous polynucleotide encoding a tolerogenic factor, and a second exogenous polynucleotide encoding one or more CARs, wherein at least one CAR comprises a CD22 antigen binding domain having the CDR sequences from SEQ ID NO: 45, 54, 85, 91, 92, or 93.

Embodiment 6. A method of treating a disease or disorder characterized by antigen evasion in a patient who has undergone one or more prior treatments for the disease or disorder prior to antigen evasion, comprising evaluating the patient for the disease or disorder characterized by antigen evasion, and administering a population of engineered CAR-T cells to the patient to treat the disease or disorder, wherein the engineered CAR-T cells comprise reduced expression of one or more MHC class I and/or class II HLA, and reduced expression of a TCR relative to an unaltered control cell, a first exogenous polynucleotide encoding a tolerogenic factor, and a second exogenous polynucleotide encoding one or more CARs, wherein at least one CAR comprises a CD22 antigen binding domain having the CDR sequences from SEQ ID NO: 45, 54, 85, 91, 92, or 93.

Embodiment 7. A method of treating a cancer characterized by antigen evasion in a patient who has undergone one or more prior treatments for the cancer prior to antigen evasion, comprising evaluating the patient for the disease or disorder characterized by antigen evasion, and administering a population of engineered CAR-T cells to the patient to treat the disease or disorder, wherein the engineered CAR-T cells comprise reduced expression of one or more MHC class I and/or class II HLA, and reduced expression of a TCR relative to an unaltered control cell, a first exogenous polynucleotide encoding a tolerogenic factor, and a second exogenous polynucleotide encoding one or more CARs, wherein at least one CAR comprises a CD22 antigen binding domain having the CDR sequences from SEQ ID NO: 45, 54, 85, 91, 92, or 93.

Embodiment 8. The method of any one of embodiments 5-7, wherein the engineered CAR-T cells comprise reduced expression of TCR-alpha (TRAC) and/or TCR-beta (TRBC).

Embodiment 9. A method of treating a disease or disorder in a patient who has undergone one or more prior treatments for the disease or disorder, comprising evaluating the patient for the disease or disorder, and administering a population of engineered CAR-T cells to the patient to treat the disease or disorder, wherein the engineered CAR-T cells comprise reduced expression of beta-2-microglobulin (B2M) and TRAC, relative to an unaltered control cell, a first exogenous polynucleotide encoding a tolerogenic factor, and a second exogenous polynucleotide encoding one or more CARs, wherein at least one CAR comprises a CD22 antigen binding domain having the CDR sequences from SEQ ID NO: 45, 54, 85, 91, 92, or 93, and wherein the disease or disorder is a cancer.

Embodiment 10. The method of embodiment 9, wherein the engineered CAR-T cells further comprise reduced expression of MHC class II HLA.

Embodiment 11. The method of embodiment 10, wherein the engineered CAR-T cells further comprise reduced expression of MHC class II transactivator (CIITA).

Embodiment 12. The method of any one of embodiments 9-11, wherein the tolerogenic factor is CD47.

Embodiment 13. A method of treating a disease or disorder in a patient who has undergone one or more prior treatments for the disease or disorder, comprising evaluating the patient for the disease or disorder, and administering a population of engineered CAR-T cells to the patient to treat the disease or disorder, wherein the engineered CAR-T cells comprise reduced expression of B2M, CIITA, and TRAC, relative to an unaltered control cell, a first exogenous polynucleotide encoding CD47, and a second exogenous polynucleotide encoding one or more CARs, wherein at least one CAR comprises a CD22 antigen binding domain having the CDR sequences from SEQ ID NO: 45, 54, 85, 91, 92, or 93, wherein the first exogenous polynucleotide and the second exogenous polynucleotide are inserted by a bicistronic vector, and wherein the disease or disorder is a cancer.

Embodiment 14. A method of treating a disease or disorder in a patient who has undergone one or more prior treatments for the disease or disorder, comprising evaluating the patient for the disease or disorder, and administering a population of engineered CAR-T cells to the patient to treat the disease or disorder, wherein the engineered CAR-T cells comprise reduced expression of one or more MHC class I and/or class II human leukocyte antigens relative to an unaltered control cell, a first exogenous polynucleotide encoding CD47, and a second exogenous polynucleotide encoding one or more CARs, wherein at least one CAR comprises a CD22 antigen binding domain having the CDR sequences from SEQ ID NO: 45, 54, 85, 91, 92, or 93, and wherein the disease or disorder is a cancer.

Embodiment 15. A method of treating a disease or disorder in a patient who has undergone one or more prior treatments for the disease or disorder, comprising evaluating the patient for the disease or disorder, and administering a population of engineered CAR-T cells to the patient to treat the disease or disorder, wherein the engineered CAR-T cells comprise reduced expression of B2M relative to an unaltered control cell, a first exogenous polynucleotide encoding a tolerogenic factor, and a second exogenous polynucleotide encoding one or more CARs, wherein at least one CAR comprises a CD22 antigen binding domain having the CDR sequences from SEQ ID NO: 45, 54, 85, 91, 92, or 93, and wherein the disease or disorder is a cancer.

Embodiment 16. A method of treating a disease or disorder in a patient who has undergone one or more prior treatments for the disease or disorder, comprising evaluating the patient for the disease or disorder, and administering a population of engineered CAR-T cells to the patient to treat the disease or disorder, wherein the engineered CAR-T cells comprise reduced expression of B2M and CIITA relative to an unaltered control cell, a first exogenous polynucleotide encoding a tolerogenic factor, and a second exogenous polynucleotide encoding one or more CARs, wherein at least one CAR comprises a CD22 antigen binding domain having the CDR sequences from SEQ ID NO: 45, 54, 85, 91, 92, or 93, and wherein the disease or disorder is a cancer.

Embodiment 17. A method of treating a disease or disorder in a patient who has undergone one or more prior treatments for the disease or disorder, comprising evaluating the patient for the disease or disorder, and administering a population of engineered CAR-T cells to the patient to treat the disease or disorder, wherein the engineered CAR-T cells comprise reduced expression of B2M relative to an unaltered control cell, a first exogenous polynucleotide encoding CD47, and a second exogenous polynucleotide encoding one or more CARs, wherein at least one CAR comprises a CD22 antigen binding domain having the CDR sequences from SEQ ID NO: 45, 54, 85, 91, 92, or 93, and wherein the disease or disorder is a cancer.

Embodiment 18. A method of treating a disease or disorder in a patient who has undergone one or more prior treatments for the disease or disorder, comprising evaluating the patient for the disease or disorder, and administering a population of engineered CAR-T cells to the patient to treat the disease or disorder, wherein the engineered CAR-T cells comprise reduced expression of B2M and CIITA relative to an unaltered control cell, a first exogenous polynucleotide encoding CD47, and a second exogenous polynucleotide encoding one or more CARs, wherein at least one CAR comprises a CD22 antigen binding domain having the CDR sequences from SEQ ID NO: 45, 54, 85, 91, 92, or 93, wherein the first exogenous polynucleotide and the second exogenous polynucleotide are inserted at the same locus, and wherein the disease or disorder is a cancer.

Embodiment 19. The method of any one of embodiments 1-18, wherein the CAR has a VH sequence at least 80% identical (e.g., at least 80%, at least 85%, at least 90%, at least 95%, at least 96%, at least 97%, at least 98%, at least 99%, or 100% identical) to the VH sequence of SEQ ID NO: 45, 54, 85, 91, 92, or 93.

Embodiment 20. The method of any one of embodiments 1-19, wherein the CAR has a VL sequence at least 80% identical (e.g., at least 80%, at least 85%, at least 90%, at least 95%, at least 96%, at least 97%, at least 98%, at least 99%, or 100% identical) to the VL sequence of SEQ ID NO: 45, 54, 85, 91, 92, or 93.

Embodiment 21. The method of any one of embodiments 1-20, wherein the CAR has an scFv sequence at least 80% identical (e.g., at least 80%, at least 85%, at least 90%, at least 95%, at least 96%, at least 97%, at least 98%, at least 99%, or 100% identical) to the scFv sequence of SEQ ID NO: 45, 54, 85, 91, 92, or 93.

Embodiment 22. The method of any one of embodiments 1-21, wherein the CAR further comprises one or more of the following components: leader sequence, CD8α signal peptide, linker, m971 binder-based scFv, CD8α hinge domain, CD8 transmembrane domain, CD28 transmembrane domain, 4-1BB costimulatory domain, CD28 signaling domain, CD137 signaling domain, CD8 signaling domain, and CD3ζ signaling domain.

Embodiment 23. The method of embodiment 22, wherein the CD22 CAR comprises a CD8α transmembrane domain or a CD28 transmembrane domain.

Embodiment 24. The method of embodiment 22, wherein the CD22 CAR comprises a CD137 signaling domain and a CD3ζ signaling domain.

Embodiment 25. The method of any embodiment 22, wherein the CD22 CAR comprises a CD28 signaling domain and a CD3ζ signaling domain.

Embodiment 26. The method of any embodiment 22, wherein the CD22 CAR comprises a CD28 signaling domain, a CD137 signaling domain, and a CD3ζ signaling domain.

Embodiment 27. The method of any one of embodiments 22-26, wherein the CD8α signal peptide comprises the sequence of SEQ ID NO: 6.

Embodiment 28. The method of any one of embodiments 22-27, wherein the linker is selected from the group consisting of IgG linkers, Whitlow linkers, (G4S)n linkers, wherein n is 1, 2, 3, 4, or more, and modifications thereof.

Embodiment 29. The method of embodiment 28, wherein the linker is a (G4S)n linker, wherein n is 1 or 3.

Embodiment 30. The method of any one of embodiments 22-29, wherein the m971 binder-based scFv comprises CDRs comprising the sequences of SEQ ID NOs: 47-49 and 51-53.

Embodiment 31. The method of any one of embodiments 22-30, wherein the m971 binder-based scFv comprises the VH and VL domains of SEQ ID NO: 45, 54, or 139.

Embodiment 32. The method of any one of embodiments 22-31, wherein the m971 binder-based scFv comprises the sequence of SEQ ID NO: 45, 54, or 139.

Embodiment 33. The method of any one of embodiments 22-32, wherein the m971 binder-based scFv comprises a binder that is functionally equivalent to the m971 binder.

Embodiment 34. The method of any one of embodiments 22-33, wherein the m971 binder-based scFv is an m971-L7-based scFv, optionally wherein the m971-L7-based ScFv comprises the sequence of SEQ ID NO: 54.

Embodiment 35. The method of any one of embodiments 22-34, wherein the CD8α hinge domain comprises the sequence of SEQ ID NO: 9.

Embodiment 36. The method of any one of embodiments 22-35, wherein the CD8 transmembrane domain comprises the sequence of SEQ ID NO: 14 or 86.

Embodiment 37. The method of any one of embodiments 22-36, wherein the CD28 transmembrane domain comprises the sequence of SEQ ID NO: 15, 87, or 114.

Embodiment 38. The method of any one of embodiments 22-37, wherein the 4-1BB costimulatory domain comprises the sequence of SEQ ID NO: 16.

Embodiment 39. The method of any one of embodiments 22-38, wherein the CD28 signaling domain comprises the sequence of SEQ ID NO: 17 or 88.

Embodiment 40. The method of any one of embodiments 22-39, wherein the CD137 signaling domain comprises the sequence of SEQ ID NO: 90.

Embodiment 41. The method of any one of embodiments 22-40, wherein the CD8 signaling domain comprises the sequence of SEQ ID NO: 89.

Embodiment 42. The method of any one of embodiments 22-41, wherein the CD3ζ signaling domain comprises the sequence of SEQ ID NO: 18 or 115.

Embodiment 43. The method of any one of embodiments 1-42, wherein the CAR comprises the sequence at least 80% identical (e.g., at least 80%, at least 85%, at least 90%, at least 95%, at least 96%, at least 97%, at least 98%, at least 99%, or 100% identical) to the amino acid sequence of SEQ ID NO: 91, 92, or 93.

Embodiment 44. The method of any one of embodiments 1-43, wherein the prior treatments are CD19-specific and/or CD20-specific prior treatments.

Embodiment 45. The method of any one of embodiments 1-44, wherein the disease or disorder is characterized by antigen evasion, and wherein the patient has undergone one or more prior treatments for the disease or disorder prior to antigen evasion.

Embodiment 46. The method of any one of embodiments 1-45, wherein the disease or disorder is cancer characterized by antigen evasion, and wherein the patient has undergone one or more prior treatments for the cancer prior to antigen evasion.

Embodiment 47. The method of any one of embodiments 1-46, wherein the patient is diagnosed as having the disease or disorder prior to administering the population of engineered CAR-T cells.

Embodiment 48. The method of any one of embodiments 1-47, wherein the prior treatment comprises an antibody-based therapy, an immune-oncology therapy, or a cell-based therapy.

Embodiment 49. The method of any one of embodiments 1-48, wherein the prior treatment comprises a cell-based therapy comprising an autologous CAR-T therapy or an allogeneic CAR-T therapy.

Embodiment 50. The method of any one of embodiments 1-49, wherein the prior treatment comprises autologous or allogeneic CAR-T cells expressing a CD22-specific CAR that is the same as, or different from, the CAR expressed by the engineered CAR-T cells.

Embodiment 51. The method of any one of embodiments 1-50, wherein the prior treatment comprises autologous or allogeneic CAR-T cells expressing a CD22-specific CAR that is functionally equivalent to the CAR expressed by the engineered CAR-T cells.

Embodiment 52. The method of any one of embodiments 1-51, wherein the prior treatment comprises autologous or allogeneic CAR-T cells expressing a CAR that is different from the CAR expressed by the engineered CAR-T cells.

Embodiment 53. The method of embodiment 52, wherein the prior treatment comprises autologous or allogeneic CD19-CAR-T cells.

Embodiment 54. The method of embodiment 53, wherein the allogeneic CD19-CAR-T cells comprise a CAR comprising the CDR sequences of SEQ ID NO: 19, 29, 32, 34, 36, 37, or 117, or a functionally equivalent CAR thereof.

Embodiment 55. The method of embodiment 53 or 54, wherein the allogeneic CD19-CAR-T cells comprise a CAR comprising the scFv sequence of SEQ ID NO: 19, 29, 32, 34, 36, 37, or 117, or a functionally equivalent CAR thereof.

Embodiment 56. The method of any one of embodiments 53-55, wherein the allogeneic CD19-CAR-T cells comprise a CAR comprising the sequence of 32, 34, 36, or 117, or a functionally equivalent CAR thereof.

Embodiment 57. The method of embodiment 53, wherein the prior treatment comprises axicabtagene ciloleucel, lisocabtagene maraleucel, brexucabtagene autoleucel, or tisagenlecleucel, or a functionally equivalent treatment thereof.

Embodiment 58. The method of any one of embodiments 1-57, wherein the prior treatment is a failed prior treatment.

Embodiment 59. The method of embodiment 58, wherein the failed prior treatment is characterized by one or more of: (a) a plateau or increase in one or more symptom of the disease, (b) a plateau or a worsening of the extent or state of the disease, (c) a plateau or a worsening of disease progression, (d) an attenuated response to therapy, and (e) disease recurrence.

Embodiment 60. The method of any one of embodiments 1-59, wherein the antigen binding domain of the one or more CARs binds to one or more antigens associated with the disease or the disorder.

Embodiment 61. The method of any one of embodiments 1-60, wherein the disease or disorder is cancer.

Embodiment 62. The method of embodiment 61, wherein the cancer is a lymphoma, such as a B cell lymphoma.

Embodiment 63. The method of any one of embodiments 1-62, wherein the patient is treated with an immunodepleting therapy prior to administering the engineered CAR-T cells.

Embodiment 64. The method of any one of embodiments 1-63, wherein the immunodepleting therapy administered prior to administering the engineered CAR-T cells is lower than the immunodepleting therapy administered to the patient prior to the prior treatment.

Embodiment 65. The method of embodiment 64, wherein the immunodepleting therapy comprises fewer doses than the immunodepleting therapy administered to the patient prior to the prior treatment.

Embodiment 66. The method of embodiment 64 or 65, wherein the immunodepleting therapy comprises a reduced amount of immunodepleting agent than the immunodepleting therapy administered to the patient prior to the prior treatment.

Embodiment 67. The method of any one of embodiments 1-66, wherein the immunodepleting therapy comprises administration of fludarabine and/or cyclophosphamide.

Embodiment 68. The method of any one of embodiments 1-67, wherein the immunodepleting therapy comprises IV infusion of about 1-50 mg/m2 of fludarabine for about 1-7 days.

Embodiment 69. The method of embodiment 68, wherein the immunodepleting therapy comprises IV infusion of about 1, about 5, about 10, about 20, about 30, about 40, or about 50 mg/m2 of fludarabine for about 1, about 2, about 3, about 4, about 5, about 6, or about 7 days.

Embodiment 70. The method of embodiment 68 or 69, wherein the immunodepleting therapy comprises IV infusion of about 30 mg/m2 of fludarabine for about 5 days.

Embodiment 71. The method of embodiment 68 or 69, wherein the immunodepleting therapy comprises IV infusion of about 30 mg/m2 of fludarabine for about 3 days.

Embodiment 72. The method of any one of embodiments 1-71, wherein the immunodepleting therapy comprises IV infusion of about 100-1000 mg/m2 of cyclophosphamide for about 1-7 days.

Embodiment 73. The method of embodiment 72, wherein the immunodepleting therapy comprises IV infusion of about 100, about 200, about 300, about 400, about 500, about 600, about 700, about 800, about 900, or about 1000 mg/m2 of cyclophosphamide for about 1, about 2, about 3, about 4, about 5, about 6, or about 7 days.

Embodiment 74. The method of embodiment 73, wherein the immunodepleting therapy comprises IV infusion of about 500 mg/m2 or more of cyclophosphamide for about 5 days.

Embodiment 75. The method of embodiment 73 or 74, wherein the immunodepleting therapy further comprises IV infusion of about 3 mg, about 10 mg, or about 30 mg of alemtuzumab for about 1, about 2, about 3, about 4, about 5, about 6, or about 7 days.

Embodiment 76. The method of embodiment 73, wherein the immunodepleting therapy comprises IV infusion of about 500 mg/m2 of cyclophosphamide for about 3 days.

Embodiment 77. The method of any one of embodiments 1-76, wherein the administration is selected from the group consisting of intravenous injection, intramuscular injection, intravascular injection, and transplantation.

Embodiment 78. The method of any one of embodiments 1-77, wherein at least about 40×104 engineered CAR-T cells are administered to the patient.

Embodiment 79. The method of any one of embodiments 1-78, wherein at least about 40×104 engineered CAR-T cells are administered to the patient.

Embodiment 80. The method of any one of embodiments 1-79, wherein up to about 8.0×108 engineered CAR-T cells are administered to the patient, optionally wherein up to about 6.0×108 engineered CAR-T cells are administered to the patient, optionally wherein about 1.0×106 to about 2.5×108 engineered CAR-T cells are administered to the patient or wherein about 2.0×106 to about 2.0×108 engineered CAR-T cells are administered to the patient.

Embodiment 81. The method of any one of embodiments 1-80, wherein up to about 6.0×108 engineered CAR-T cells are administered to the patient in about 1-3 doses, optionally wherein (a) about 0.6×106 to about 6.0×108 engineered CAR-T cells are administered to the patient in about 1-3 doses, (b) about 0.2×106 to about 5.0×106 engineered CAR-T cells per kg of the patient's body weight are administered to the patient in about 1-3 doses, if the patient has a body weight of 50 kg or less, (c) about 0.1×108 to about 2.5×108 engineered CAR-T cells are administered to the patient in about 1-3 doses, if the patient has a body weight greater than 50 kg, or (d) about 2.0×106 engineered CAR-T cells per kg of the patient's body weight and up to about 2.0×108 engineered CAR-T cells are administered to the patient in about 1-3 doses.

Embodiment 82. The method of any one of embodiments 1-81, wherein about 40×106 to about 200×106 engineered CAR-T cells are administered to the patient, optionally wherein (a) about 40×106 to about 60×106 engineered CAR-T cells are administered to the patient, (b) about 60×106 to about 80×106 engineered CAR-T cells are administered to the patient, (c) about 80×106 to about 100×106 engineered CAR-T cells are administered to the patient, (d) about 100×106 to about 120×106 engineered CAR-T cells are administered to the patient, (e) about 120×106 to about 140×106 engineered CAR-T cells are administered to the patient, (f) about 140×106 to about 160×106 engineered CAR-T cells are administered to the patient, (g) about 160×106 to about 180×106 engineered CAR-T cells are administered to the patient, or (h) about 180×106 to about 200×106 engineered CAR-T cells are administered to the patient.

Embodiment 83. The method of any one of embodiments 1-82, wherein about 60×106 to about 120×106 engineered CAR-T cells are administered to the patient, optionally wherein (a) about 60×106 to about 80×106 engineered CAR-T cells are administered to the patient, (b) about 80×106 to about 100×106 engineered CAR-T cells are administered to the patient, or (c) about 100×106 to about 120×106 engineered CAR-T cells are administered to the patient.

Embodiment 84. The method of any one of embodiments 1-83, wherein about 120×106 to about 200×106 engineered CAR-T cells are administered to the patient, (a) about 120×106 to about 140×106 engineered CAR-T cells are administered to the patient, (b) about 140×106 to about 160×106 engineered CAR-T cells are administered to the patient, (c) about 160×106 to about 180×106 engineered CAR-T cells are administered to the patient, or (d) about 180×106 to about 200×106 engineered CAR-T cells are administered to the patient.

Embodiment 85. The method of any one of embodiments 1-84, wherein the prior treatment comprises an autologous or allogeneic cell-based therapy, and wherein fewer or a lower number of engineered CAR-T cells are administered to the patient than were included in the prior therapy.

Embodiment 86. The method of any one of embodiments 1-85, further comprising administering a second, third, fourth, fifth, or sixth dose of the engineered CAR-T cells to the patient.

Embodiment 87. The method of embodiment 86, wherein the patient is not treated with an immunodepleting therapy prior to the second, third, fourth, fifth, and/or sixth administration of the engineered CAR-T cells.

Embodiment 88. The method of embodiment 86, wherein the patient is treated with an immunodepleting therapy prior to the second, third, fourth, fifth, and/or sixth administration of the engineered CAR-T cells.

Embodiment 89. The method of embodiment 88, wherein the immunodepleting therapy that is administered prior to the second, third, fourth, fifth, and/or sixth administration of the engineered CAR-T cells is independently selected from administration of fludarabine and/or cyclophosphamide, wherein the administration of fludarabine comprises IV infusion of about 1-50 mg/m2 of fludarabine for about 1-7 days, and the administration of cyclophosphamide comprises IV infusion of about 100-1000 mg/m2 of cyclophosphamide for about 1-7 days.

Embodiment 90. The method of any one of embodiments 1-89, wherein the engineered CAR-T cells are propagated from a primary T cell or a progeny thereof, or are derived from a T cell differentiated from an iPSC or a progeny thereof.

Embodiment 91. The method of any one of embodiments 1-90, wherein the engineered CAR-T cells are differentiated cells derived from an induced pluripotent stem cell or a progeny thereof.

Embodiment 92. The method of embodiment 91, wherein the differentiated cells are a T cells or natural killer (NK) cells.

Embodiment 93. The method of any one of embodiments 1-90, wherein the engineered CAR-T cells are a progeny of primary immune cells.

Embodiment 94. The method of embodiment 93, wherein the progeny of primary immune cells are T cells or NK cells.

Embodiment 95. The method of any one of embodiments 1-94, wherein the wild type cell or the control cell is a starting material.

Embodiment 96. The method of any one of embodiments 1-95, wherein the engineered CAR-T cells are CAR+ T cells that comprise any one selected from the group consisting of a bulk population of CAR+ T cells, CD4+ CAR+ T cells, CD8+ CAR+ T cells, and a combination thereof.

Embodiment 97. The method of embodiment 96, wherein the CD4+ CAR+ T cells and CD8+ CAR+ T cells are administered concomitantly or sequentially.

Embodiment 98. The method of embodiment 97, wherein the CD4+ CAR+ T cells are administered prior to administration of the CD8+ CAR+ T cells, or wherein the CD8+ CAR+ T cells are administered prior to administration of the CD4+ CAR+ T cells.

Embodiment 99. The method of embodiment 96, wherein the bulk CAR+ T cells and CD8+ CAR+ T cells are administered concomitantly or sequentially.

Embodiment 100. The method of embodiment 99, wherein the bulk CAR+ T cells are administered prior to administration of the CD8+ CAR+ T cells, or wherein the CD8+ CAR+ T cells are administered prior to administration of the bulk CAR+ T cells.

Embodiment 101. The method of embodiment 96, wherein the CD4+ CAR+ T cells and bulk CAR+ T cells are administered concomitantly or sequentially.

Embodiment 102. The method of embodiment 101, wherein the CD4+ CAR+ T cells are administered prior to administration of the bulk CAR+ T cells, or wherein the bulk CAR+ T cells are administered prior to administration of the CD4+ CAR+ T cells.

Embodiment 103. The method of any one of embodiments 1-102, wherein the engineered CAR-T cells comprise reduced expression of B2M and/or CIITA relative to an unaltered control cell.

Embodiment 104. The method of embodiment 103, wherein the engineered CAR-T cells do not express B2M and/or CIITA.

Embodiment 105. The method of any one of embodiments 1-104, wherein the engineered CAR-T cells comprise reduced expression of a TCR.

Embodiment 106. The method of embodiment 105, wherein the engineered CAR-T cells comprise reduced expression of TRAC and/or TRBC.

Embodiment 107. The method of embodiment 105 or 106, wherein the engineered CAR-T cells do not express TRAC and/or TRBC.

Embodiment 108. The method of any one of embodiments 1-107, wherein the engineered CAR-T cells comprise reduced expression of HLA class I antigens and/or HLA class II antigens relative to an unaltered control cell.

Embodiment 109. The method of embodiment 108, wherein the engineered CAR-T cells do not express HLA class I antigens, HLA class 11 antigens, and/or do not express TCR-alpha.

Embodiment 110. The method of embodiment 108 or 109, wherein the reduced expression or no expression of HLA class I antigens results from the reduced expression or no expression of B2M, and where in the reduced expression or no expression of HLA class 11 antigens results from the reduced expression or no expression of CIITA.

Embodiment 111. The method of any one of embodiments 1-110, wherein the engineered CAR-T cells are B2M^{indel/indel}, CliTAindel/indel cell, and/or a TRAC^{indel/indel}, and/or TRAC^{indel/indel} cells.

Embodiment 112. The method of any one of embodiments 1-111, wherein the engineered CAR-T cells comprise reduced expression of HLA-A, HLA-B, HLA-C, HLA-DP, HLA-DQ, HLA-DR, RHD, ABO, PCDH11Y, and/or NLGN4Y relative to an unaltered control cell.

Embodiment 113. The method of embodiment 112, wherein the engineered CAR-T cells do not express HLA-A, HLA-B, HLA-C, HLA-DP, HLA-DQ, HLA-DR, RHD, ABO, PCDH11Y, and/or NLGN4Y.

Embodiment 114. The method of any one of embodiments 1-113, wherein the reduced expression is by way of gene knock down, optionally wherein the gene knock down is by way of RNA silencing or RNA interference (RNAi), optionally selected from the group consisting of short interfering RNAs (siRNAs), PIWI-interacting RNAs (piRNAs), short hairpin RNAs (shRNAs), and microRNAs (miRNAs).

Embodiment 115. The method of any one of embodiments 1-114, wherein the reduced expression is by way of gene knock out, optionally wherein the gene knock out is by way of inducing an insertion or a deletion in the gene using a gene editing system, wherein the gene editing system is optionally selected from the group consisting of zinc finger nucleases (ZFNs), transcription activator-like effector nucleases (TALENs), meganucleases, transposases, clustered regularly interspaced short palindromic repeat (CRISPR)/Cas systems, nickase systems, base editing systems, prime editing systems, and gene writing systems.

Embodiment 116. The method of any one of embodiments 1-115, wherein the one or more tolerogenic factors are selected from the group consisting of CD47, CD24, CD27, CD35, CD46, CD55, CD59, CD200, HLA-C, HLA-E, HLA-E heavy chain, HLA-G, PD-L1, IDO1, CTLA4-Ig, C1-Inhibitor (e.g., CR1), IL-10, IL-35, FasL, CCL21, CCL22, Mfge8, and Serpinb9.

Embodiment 117. The method of embodiment 116, wherein the one or more tolerogenic factors comprise CD47.

Embodiment 118. A method of treating a disease or disorder in a patient who has undergone one or more prior treatments for the disease or disorder, comprising evaluating the patient for the disease or disorder, and administering a population of engineered CAR-T cells to the patient to treat the disease or disorder, wherein the engineered CAR-T cells comprise reduced expression of B2M and TRAC, relative to an unaltered control cell, a first exogenous polynucleotide encoding HLA-E, and a second exogenous polynucleotide encoding one or more CARs, wherein at least one CAR comprises a CD22 antigen binding domain having the CDR sequences from SEQ ID NO: 45, 54, 85, 91, 92, or 93, and wherein the disease or disorder is a cancer.

Embodiment 119. The method of embodiment 118, wherein the HLA-E is a single chain trimer.

Embodiment 120. The method of embodiment 118, wherein the HLA-E is a HLA-E/B2M fusion.

Embodiment 121. A method of treating a disease or disorder in a patient who has undergone one or more prior treatments for the disease or disorder, comprising evaluating the patient for the disease or disorder, and administering a population of engineered CAR-T cells to the patient to treat the disease or disorder, wherein the engineered CAR-T cells comprise reduced expression of B2M and/or CR-1 and TRAC, relative to an unaltered control cell, a first exogenous polynucleotide encoding CD24, and a second exogenous polynucleotide encoding one or more CARs, wherein at least one CAR comprises a CD22 antigen binding domain having the CDR sequences from SEQ ID NO: 45, 54, 85, 91, 92, or 93, and wherein the disease or disorder is a cancer.

Embodiment 122. A method of treating a disease or disorder in a patient who has undergone one or more prior treatments for the disease or disorder, comprising evaluating the patient for the disease or disorder, and administering a population of engineered CAR-T cells to the patient to treat the disease or disorder, wherein the engineered CAR-T cells comprise reduced expression of B2M and/or CD52 and TRAC, relative to an unaltered control cell, optionally a first exogenous polynucleotide encoding a tolerogenic factor, and a second exogenous polynucleotide encoding one or more CARs, wherein at least one CAR comprises a CD22 antigen binding domain having the CDR sequences from SEQ ID NO: 45, 54, 85, 91, 92, or 93, and wherein the disease or disorder is a cancer.

Embodiment 123. A method of treating a disease or disorder in a patient who has undergone one or more prior treatments for the disease or disorder, comprising evaluating the patient for the disease or disorder, and administering a population of engineered CAR-T cells to the patient to treat the disease or disorder, wherein the engineered CAR-T cells comprise reduced expression of B2M and/or CD70 and TRAC, relative to an unaltered control cell, optionally a first exogenous polynucleotide encoding a tolerogenic factor, and a second exogenous polynucleotide encoding one or more CARs, wherein at least one CAR comprises a CD22 antigen binding domain having the CDR sequences from SEQ ID NO: 45, 54, 85, 91, 92, or 93, and wherein the disease or disorder is a cancer.

Embodiment 124. A method of treating a disease or disorder in a patient who has undergone one or more prior treatments for the disease or disorder, comprising evaluating the patient for the disease or disorder, and administering a population of engineered CAR-T cells to the patient to treat the disease or disorder, wherein the engineered CAR-T cells comprise reduced expression of PD-1 and TRAC, relative to an unaltered control cell, optionally a first exogenous polynucleotide encoding a tolerogenic factor, and a second exogenous polynucleotide encoding one or more CARs, wherein at least one CAR comprises a CD22 antigen binding domain having the CDR sequences from SEQ ID NO: 45, 54, 85, 91, 92, or 93, and wherein the disease or disorder is a cancer.

Embodiment 125. The method of any one of embodiments 1-124, wherein the engineered CAR-T cells comprise a third exogenous polynucleotide encoding a CD19-specific CAR.

Embodiment 126. The method of embodiment 125, wherein the CD19-specific CAR comprises a hinge domain of any one of SEQ ID NOs: 9-13, a transmembrane sequence of any one of SEQ ID NOs: 14, 15, and 114, and/or an intracellular costimulatory and/or signaling domain of any one of SEQ ID NOs: 16-18 and 115.

Embodiment 127. The method of any one of embodiments 1-126, wherein the first exogenous polynucleotide, the second exogenous polynucleotide, and/or the third exogenous polynucleotides are carried by a polycistronic vector.

Embodiment 128. The method of any one of embodiments 1-127, wherein the CD22-specific CAR, the one or more tolerogenic factors, and/or the additional CD19-specific CAR are carried by a single polycistronic vector.

Embodiment 129. The method of embodiment 127 or 128, wherein the polycistronic vector is a bicistronic vector.

Embodiment 130. The method of any one of embodiments 1-129, wherein the first, second, and/or third exogenous polynucleotide, and/or the polycistronic vector is inserted into a first, second, and/or third specific locus of at least one allele of the cell.

Embodiment 131. The method of embodiment 130, wherein the first, second, and/or third specific loci are selected from the group consisting of a safe harbor locus, a target locus, an RHD locus, a B2M locus, a CIITA locus, a TRAC locus, and a TRB locus.

Embodiment 132. The method of embodiment 131, wherein the safe harbor locus is selected from the group consisting of a CCR5 locus, a PPP1R12C locus, a CLYBL locus, and a Rosa locus.

Embodiment 133. The method of embodiment 131, wherein the target locus is selected from the group consisting of a CXCR4 locus, an ALB locus, a SHS231 locus, an F3 (CD142) locus, a MICA locus, a MICB locus, a LRP1 (CD91) locus, a HMGB1 locus, an ABO locus, a FUT1 locus, and a KDM5D locus.

Embodiment 134. A method of treating a disease or disorder in a patient who has undergone one or more prior treatments for the disease or disorder, comprising evaluating the patient for the disease or disorder, and administering a population of engineered CAR-T cells to the patient to treat the disease or disorder, wherein the engineered CAR-T cells comprise reduced expression of B2M, CIITA, and TRAC, relative to an unaltered control cell, a first exogenous polynucleotide encoding CD47, and a second exogenous polynucleotide encoding a CD22 CAR comprising a sequence having at least 90% sequence homology to the sequence set forth in SEQ ID NO: 91, wherein the first exogenous polynucleotide and the second exogenous polynucleotide are inserted by a bicistronic vector, and wherein the disease or disorder is a cancer.

Embodiment 135. A method of treating a disease or disorder in a patient who has undergone one or more prior treatments for the disease or disorder, comprising evaluating the patient for the disease or disorder, and administering a population of engineered CAR-T cells to the patient to treat the disease or disorder, wherein the engineered CAR-T cells comprise reduced expression of B2M, CIITA, and TRAC, relative to an unaltered control cell, a first exogenous polynucleotide encoding CD47, and a second exogenous polynucleotide encoding a CD22 CAR comprising the sequence set forth in SEQ ID NO: 91, wherein the first exogenous polynucleotide and the second exogenous polynucleotide are inserted by a bicistronic vector, and wherein the disease or disorder is a cancer.

Embodiment 136. A method of treating a disease or disorder in a patient who has undergone one or more prior treatments for the disease or disorder, comprising evaluating the patient for the disease or disorder, and administering a population of engineered CAR-T cells to the patient to treat the disease or disorder, wherein the engineered CAR-T cells comprise reduced expression of B2M, CIITA, and TRAC, relative to an unaltered control cell, a first exogenous polynucleotide encoding CD47, a second exogenous polynucleotide encoding a CD22 CAR comprising a sequence having at least 90% sequence homology to the sequence set forth in SEQ ID NO: 91, and a third exogenous polynucleotide encoding a CD19 CAR comprising a sequence having at least 90% sequence homology to the sequence set forth in SEQ ID NO: 117 and wherein the disease or disorder is a cancer.

Embodiment 137. A method of treating a disease or disorder in a patient who has undergone one or more prior treatments for the disease or disorder, comprising evaluating the patient for the disease or disorder, and administering a population of engineered CAR-T cells to the patient to treat the disease or disorder, wherein the engineered CAR-T cells comprise reduced expression of B2M, CIITA, and TRAC, relative to an unaltered control cell, a first exogenous polynucleotide encoding CD47, a second exogenous polynucleotide encoding a CD22 CAR comprising the sequence set forth in SEQ ID NO: 91, and a third exogenous polynucleotide encoding a CD19 CAR comprising a sequence having at least 90% sequence homology to the sequence set forth in SEQ ID NO: 117 and wherein the disease or disorder is a cancer.

Embodiment 138. The method of any one of embodiments 1-137, wherein the first exogenous polynucleotide, the second exogenous polynucleotide, and/or the third exogenous polynucleotides are carried by a polycistronic vector.

Embodiment 139. The method of embodiment 138, wherein the polycistronic vector is a bicistronic vector.

Embodiment 140. The method of any one of embodiments 1-139, wherein the first, second, and/or third exogenous polynucleotide or the polycistronic vector is introduced into the engineered CAR-T cells using CRISPR/Cas gene editing.

Embodiment 141. The method of embodiment 140, wherein the CRISPR/Cas gene editing is carried out ex vivo from a donor patient.

Embodiment 142. The method of any one of embodiments 1-141, wherein the first, second, and/or third exogenous polynucleotide, and/or the polycistronic vector is inserted into at least one allele of the engineered CAR-T cell using viral transduction.

Embodiment 143. The method of embodiment 142, wherein the viral transduction includes a lentivirus based viral vector.

Embodiment 144. The method of embodiment 143, wherein the lentivirus based viral vector is a pseudotyped, self-inactivating lentiviral vector that carries the first, second, and/or third exogenous polynucleotide, and/or the polycistronic vector.

Embodiment 145. The method of any one of embodiments 1-144, wherein the lentivirus based viral vector is a pseudotyped, self-inactivating lentiviral vector that carries the first and second exogenous polynucleotides.

Embodiment 146. The method of any one of embodiments 1-145, wherein the lentiviral vector comprises the first exogenous polynucleotide followed by the second exogenous polynucleotide.

Embodiment 147. The method of any one of embodiments 1-146, wherein the lentiviral vector comprises the second exogenous polynucleotide followed by the first exogenous polynucleotide.

Embodiment 148. The method of embodiment 143-147, wherein the lentivirus based viral vector is a self-inactivating lentiviral vector pseudotyped with a vesicular stomatitis VSV-G envelope and carries the first, second, and/or third exogenous polynucleotide, and/or the polycistronic vector.

Embodiment 149. The method of any one of embodiments 143-148, wherein the CD22-specific CAR and/or the CD19-specific CAR are inserted using one or more lentiviral vectors, and the CD47 is inserted using another lentiviral vector.

Embodiment 150. The method of any one of embodiments 143-148, wherein the CD22-specific CAR and/or the CD19-specific CAR are inserted using one or more lentiviral vectors, and the CD47 is inserted using a locus-specific insertion method, optionally a CRISPR/Cas or a TALEN method.

Embodiment 151. The method of any one of embodiments 143-148, wherein the CD22-specific CAR and/or the CD19-specific CAR are inserted using a locus-specific insertion method, optionally a CRISPR/Cas or a TALEN method, and the CD47 is inserted using a lentiviral vector.

Embodiment 152. The method of any one of embodiments 143-148, wherein the CD22-specific CAR and/or the CD19-specific CAR and the CD47 are inserted using one or more lentiviral vectors.

Embodiment 153. The method of any one of embodiments 143-148, wherein the CD22-specific CAR and/or the CD19-specific CAR and the CD47 are inserted using a locus-specific insertion method, optionally a CRISPR/Cas or a TALEN method.

Embodiment 154. The method of any one of embodiments 1-153, wherein the engineered CAR-T cells evade NK cell mediated cytotoxicity upon administration to the patient.

Embodiment 155. The method of any one of embodiments 1-154, wherein the engineered CAR-T cells are protected from cell lysis by mature NK cells upon administration to the patient.

Embodiment 156. The method of any one of embodiments 1-155, wherein the engineered CAR-T cells evade macrophage-mediated cytotoxicity, optionally wherein the macrophage-mediated cytotoxicity involves phagocytosis and/or reactive oxygen species.

Embodiment 157. The method of any one of embodiments 1-156, wherein the engineered CAR-T cells do not induce an immune response to the cell upon administration to the patient.

Embodiment 158. The method of any one of embodiments 1-157, wherein the engineered CAR-T cells persist in the patient for at least 4 weeks, at least 2 months, at least 3 months, at least 4 months, at least 5 months, at least 6 months, at least 7 months, at least 8 months, at least 9 months, at least 10 months, at least 11 months, at least 12 months, or longer.

Embodiment 159. The method of any one of embodiments 1-158, wherein the prior treatment comprises an autologous or allogeneic cell-based therapy, and wherein the engineered CAR-T cells persist in the patient for longer than the cells of the prior therapy.

Embodiment 160. The method of any one of embodiments 1-159, wherein the therapeutic effect of the engineered CAR-T cells lasts for a duration of at least 4 weeks, at least 2 months, at least 3 months, at least 4 months, at least 5 months, at least 6 months, at least 7 months, at least 8 months, at least 9 months, at least 10 months, at least 11 months, at least 12 months, or longer.

Embodiment 161. The method of any one of embodiments 1-160, wherein the therapeutic effect of the engineered CAR-T cells lasts for longer than that of the prior therapy.

Embodiment 162. Use of a population of engineered CAR-T cells for treating a disease or disorder in a patient who has undergone one or more prior treatments for the disease or disorder, wherein the engineered CAR-T cells comprise an exogenous polynucleotide encoding one or more CARs, wherein at least one CAR comprises a CD22 antigen binding domain having the CDR sequences from SEQ ID NO: 45, 54, 85, 91, 92, or 93.

Embodiment 163. Use of a population of engineered CAR-T cells for treating a disease or disorder characterized by antigen evasion in a patient who has undergone one or more prior treatments for the disease or disorder prior to antigen evasion, wherein the engineered CAR-T cells comprise an exogenous polynucleotide encoding one or more CARs, wherein at least one CAR comprises a CD22 antigen binding domain having the CDR sequences from SEQ ID NO: 45, 54, 85, 91, 92, or 93.

Embodiment 164. Use of a population of engineered CAR-T cells for treating a cancer characterized by antigen evasion in a patient who has undergone one or more prior treatments for the cancer prior to antigen evasion, wherein the engineered CAR-T cells comprise an exogenous polynucleotide encoding one or more CARs, wherein at least one CAR comprises a CD22 antigen binding domain having the CDR sequences from SEQ ID NO: 45, 54, 85, 91, 92, or 93.

Embodiment 165. Use of a population of engineered CAR-T cells for treating a disease or disorder in a patient who has undergone one or more prior treatments for the disease or disorder, wherein the engineered CAR-T cells comprise reduced expression of one or more MHC class I and/or class II HLAs, and reduced expression of a TCR relative to an unaltered control cell, and a first exogenous polynucleotide encoding one or more CARs, wherein at least one CAR comprises a CD22 antigen binding domain having the CDR sequences from SEQ ID NO: 45, 54, 85, 91, 92, or 93.

Embodiment 166. Use of a population of engineered CAR-T cells for treating a disease or disorder in a patient who has undergone one or more prior treatments for the disease or disorder, wherein the engineered CAR-T cells comprise reduced expression of one or more MHC class I and/or class II HLA, and reduced expression of a TCR relative to an unaltered control cell, a first exogenous polynucleotide encoding a tolerogenic factor, and a second exogenous polynucleotide encoding one or more CARs, wherein at least one CAR comprises a CD22 antigen binding domain having the CDR sequences from SEQ ID NO: 45, 54, 85, 91, 92, or 93.

Embodiment 167. Use of a population of engineered CAR-T cells for treating a disease or disorder characterized by antigen evasion in a patient who has undergone one or more prior treatments for the disease or disorder prior to antigen evasion, wherein the engineered CAR-T cells comprise reduced expression of one or more MHC class I and/or class II HLA, and reduced expression of a TCR relative to an unaltered control cell, a first exogenous polynucleotide encoding a tolerogenic factor, and a second exogenous polynucleotide encoding one or more CARs, wherein at least one CAR comprises a CD22 antigen binding domain having the CDR sequences from SEQ ID NO: 45, 54, 85, 91, 92, or 93.

Embodiment 168. Use of a population of engineered CAR-T cells for treating a cancer characterized by antigen evasion in a patient who has undergone one or more prior treatments for the cancer prior to antigen evasion, wherein the engineered CAR-T cells comprise reduced expression of one or more MHC class I and/or class II HLA, and reduced expression of a TCR relative to an unaltered control cell, a first exogenous polynucleotide encoding a tolerogenic factor, and a second exogenous polynucleotide encoding one or more CARs, wherein at least one CAR comprises a CD22 antigen binding domain having the CDR sequences from SEQ ID NO: 45, 54, 85, 91, 92, or 93.

Embodiment 169. The use of any one of embodiments 166-168, wherein the engineered CAR-T cells comprise reduced expression of TRAC and/or TRBC.

Embodiment 170. Use of a population of engineered CAR-T cells for treating a disease or disorder in a patient who has undergone one or more prior treatments for the disease or disorder, wherein the engineered CAR-T cells comprise reduced expression of B2M and TRAC, relative to an unaltered control cell, a first exogenous polynucleotide encoding a tolerogenic factor, and a second exogenous polynucleotide encoding one or more CARs, wherein at least one CAR comprises a CD22 antigen binding domain having the CDR sequences from SEQ ID NO: 45, 54, 85, 91, 92, or 93, and wherein the disease or disorder is a cancer.

Embodiment 171. The use of embodiment 170, wherein the engineered CAR-T cells further comprise reduced expression of MHC class II HLA.

Embodiment 172. The use of embodiment 171, wherein the engineered CAR-T cells further comprise reduced expression of CIITA.

Embodiment 173. The use of any one of embodiments 170-172, wherein the tolerogenic factor is CD47.

Embodiment 174. Use of a population of engineered CAR-T cells for treating a disease or disorder in a patient who has undergone one or more prior treatments for the disease or disorder, wherein the engineered CAR-T cells comprise reduced expression of B2M, CIITA, and TRAC, relative to an unaltered control cell, a first exogenous polynucleotide encoding CD47, and a second exogenous polynucleotide encoding one or more CARs, wherein at least one CAR comprises a CD22 antigen binding domain having the CDR sequences from SEQ ID NO: 45, 54, 85, 91, 92, or 93, wherein the first exogenous polynucleotide and the second exogenous polynucleotide are inserted by a bicistronic vector, and wherein the disease or disorder is a cancer.

Embodiment 175. Use of a population of engineered CAR-T cells for treating a disease or disorder in a patient who has undergone one or more prior treatments for the disease or disorder, wherein the engineered CAR-T cells comprise reduced expression of one or more MHC class I and/or class II human leukocyte antigens relative to an unaltered control cell, a first exogenous polynucleotide encoding CD47, and a second exogenous polynucleotide encoding one or more CARs, wherein at least one CAR comprises a CD22 antigen binding domain having the CDR sequences from SEQ ID NO: 45, 54, 85, 91, 92, or 93, and wherein the disease or disorder is a cancer.

Embodiment 176. Use of a population of engineered CAR-T cells for treating a disease or disorder in a patient who has undergone one or more prior treatments for the disease or disorder, wherein the engineered CAR-T cells comprise reduced expression of B2M relative to an unaltered control cell, a first exogenous polynucleotide encoding a tolerogenic factor, and a second exogenous polynucleotide encoding one or more CARs, wherein at least one CAR comprises a CD22 antigen binding domain having the CDR sequences from SEQ ID NO: 45, 54, 85, 91, 92, or 93, and wherein the disease or disorder is a cancer.

Embodiment 177. Use of a population of engineered CAR-T cells for treating a disease or disorder in a patient who has undergone one or more prior treatments for the disease or disorder, wherein the engineered CAR-T cells comprise reduced expression of B2M and CIITA relative to an unaltered control cell, a first exogenous polynucleotide encoding a tolerogenic factor, and a second exogenous polynucleotide encoding one or more CARs, wherein at least one CAR comprises a CD22 antigen binding domain having the CDR sequences from SEQ ID NO: 45, 54, 85, 91, 92, or 93, and wherein the disease or disorder is a cancer.

Embodiment 178. Use of a population of engineered CAR-T cells for treating a disease or disorder in a patient who has undergone one or more prior treatments for the disease or disorder, wherein the engineered CAR-T cells comprise reduced expression of B2M relative to an unaltered control cell, a first exogenous polynucleotide encoding CD47, and a second exogenous polynucleotide encoding one or more CARs, wherein at least one CAR comprises a CD22 antigen binding domain having the CDR sequences from SEQ ID NO: 45, 54, 85, 91, 92, or 93, and wherein the disease or disorder is a cancer.

Embodiment 179. Use of a population of engineered CAR-T cells for treating a disease or disorder in a patient who has undergone one or more prior treatments for the disease or disorder, wherein the engineered CAR-T cells comprise reduced expression of B2M and CIITA relative to an unaltered control cell, a first exogenous polynucleotide encoding CD47, and a second exogenous polynucleotide encoding one or more CARs, wherein at least one CAR comprises a CD22 antigen binding domain having the CDR sequences from SEQ ID NO: 45, 54, 85, 91, 92, or 93, wherein the first exogenous polynucleotide and the second exogenous polynucleotide are inserted at the same locus, and wherein the disease or disorder is a cancer.

Embodiment 180. The use of any one of embodiments 162-179, wherein the CAR has a VH sequence at least 80% identical (e.g., at least 80%, at least 85%, at least 90%, at least 95%, at least 96%, at least 97%, at least 98%, at least 99%, or 100% identical) to the VH sequence of SEQ ID NO: 45, 54, 85, 91, 92, or 93.

Embodiment 181. The use of any one of embodiments 162-180, wherein the CAR has a VL sequence at least 80% identical (e.g., at least 80%, at least 85%, at least 90%, at least 95%, at least 96%, at least 97%, at least 98%, at least 99%, or 100% identical) to the VL sequence of SEQ ID NO: 45, 54, 85, 91, 92, or 93.

Embodiment 182. The use of any one of embodiments 162-181, wherein the CAR has an scFv sequence at least 80% identical (e.g., at least 80%, at least 85%, at least 90%, at least 95%, at least 96%, at least 97%, at least 98%, at least 99%, or 100% identical) to the scFv sequence of SEQ ID NO: 45, 54, 85, 91, 92, or 93.

Embodiment 183. The use of any one of embodiments 162-182, wherein the CAR further comprises one or more of the following components: leader sequence, CD8α signal peptide, linker, m971 binder-based scFv, CD8α hinge domain, CD8 transmembrane domain, CD28 transmembrane domain, 4-1BB costimulatory domain, CD28 signaling domain, CD137 signaling domain, CD8 signaling domain, and CD3ζ signaling domain.

Embodiment 184. The use of embodiment 183, wherein the CD22 CAR comprises a CD8α transmembrane domain or a CD28 transmembrane domain.

Embodiment 185. The use of embodiment 183, wherein the CD22 CAR comprises a CD137 signaling domain and a CD3ζ signaling domain.

Embodiment 186. The use of any embodiment 183, wherein the CD22 CAR comprises a CD28 signaling domain and a CD3ζ signaling domain.

Embodiment 187. The use of any embodiment 183, wherein the CD22 CAR comprises a CD28 signaling domain, a CD137 signaling domain, and a CD3ζ signaling domain.

Embodiment 188. The use of any one of embodiments 183-187, wherein the CD8α signal peptide comprises the sequence of SEQ ID NO: 6.

Embodiment 189. The use of any one of embodiments 183-188, wherein the linker is selected from the group consisting of IgG linkers, Whitlow linkers, (G4S)n linkers, wherein n is 1, 2, 3, 4, or more, and modifications thereof.

Embodiment 190. The use of embodiment 189, wherein the linker is a (G4S)n linker, wherein n is 1 or 3.

Embodiment 191. The use of any one of embodiments 183-190, wherein the m971 binder-based scFv comprises CDRs comprising the sequences of SEQ ID NOs: 47-49 and 51-53.

Embodiment 192. The use of any one of embodiments 183-191, wherein the m971 binder-based scFv comprises the VH and VL domains of SEQ ID NO: 45, 54, or 139.

Embodiment 193. The use of any one of embodiments 183-192, wherein the m971 binder-based scFv comprises the sequence of SEQ ID NO: 45, 54, or 139.

Embodiment 194. The use of any one of embodiments 183-193, wherein the m971 binder-based scFv comprises a binder that is functionally equivalent to the m971 binder.

Embodiment 195. The use of any one of embodiments 183-194, wherein the m971 binder-based scFv is an m971-L7-based scFv, optionally wherein the m971-L7-based ScFv comprises the sequence of SEQ ID NO: 54.

Embodiment 196. The use of any one of embodiments 183-195, wherein the CD8α hinge domain comprises the sequence of SEQ ID NO: 9.

Embodiment 197. The use of any one of embodiments 183-196, wherein the CD8 transmembrane domain comprises the sequence of SEQ ID NO: 14 or 86.

Embodiment 198. The use of any one of embodiments 183-197, wherein the CD28 transmembrane domain comprises the sequence of SEQ ID NO: 15, 87, or 114.

Embodiment 199. The use of any one of embodiments 183-198, wherein the 4-1BB costimulatory domain comprises the sequence of SEQ ID NO: 16.

Embodiment 200. The use of any one of embodiments 183-199, wherein the CD28 signaling domain comprises the sequence of SEQ ID NO: 17 or 88.

Embodiment 201. The use of any one of embodiments 183-200, wherein the CD137 signaling domain comprises the sequence of SEQ ID NO: 90.

Embodiment 202. The use of any one of embodiments 183-201, wherein the CD8 signaling domain comprises the sequence of SEQ ID NO: 89.

Embodiment 203. The use of any one of embodiments 183-202, wherein the CD3ζ signaling domain comprises the sequence of SEQ ID NO: 18 or 115.

Embodiment 204. The use of any one of embodiments 162-203, wherein the CAR comprises the sequence at least 80% identical (e.g., at least 80%, at least 85%, at least 90%, at least 95%, at least 96%, at least 97%, at least 98%, at least 99%, or 100% identical) to the amino acid sequence of SEQ ID NO: 91, 92, or 93.

Embodiment 205. The use of any one of embodiments 162-204, wherein the prior treatments are CD19-specific and/or CD20-specific prior treatments.

Embodiment 206. The use of any one of embodiments 162-205, wherein the disease or disorder is characterized by antigen evasion, and wherein the patient has undergone one or more prior treatments for the disease or disorder prior to antigen evasion.

Embodiment 207. The use of any one of embodiments 162-206, wherein the disease or disorder is cancer characterized by antigen evasion, and wherein the patient has undergone one or more prior treatments for the cancer prior to antigen evasion.

Embodiment 208. The use of any one of embodiments 162-207, wherein the patient is diagnosed as having the disease or disorder prior to administering the population of engineered CAR-T cells.

Embodiment 209. The use of any one of embodiments 162-208, wherein the prior treatment comprises an antibody-based therapy, an immune-oncology therapy, or a cell-based therapy.

Embodiment 210. The use of any one of embodiments 162-209, wherein the prior treatment comprises a cell-based therapy comprising an autologous CAR-T therapy or an allogeneic CAR-T therapy.

Embodiment 211. The use of any one of embodiments 162-210, wherein the prior treatment comprises autologous or allogeneic CAR-T cells expressing a CD22-specific CAR that is the same as, or different from, the CAR expressed by the engineered CAR-T cells.

Embodiment 212. The use of any one of embodiments 162-211, wherein the prior treatment comprises autologous or allogeneic CAR-T cells expressing a CD22-specific CAR that is functionally equivalent to the CAR expressed by the engineered CAR-T cells.

Embodiment 213. The use of any one of embodiments 162-212, wherein the prior treatment comprises autologous or allogeneic CAR-T cells expressing a CAR that is different from the CAR expressed by the engineered CAR-T cells.

Embodiment 214. The use of embodiment 213, wherein the prior treatment comprises autologous or allogeneic CD19-CAR-T cells.

Embodiment 215. The use of embodiment 214, wherein the allogeneic CD19-CAR-T cells comprise a CAR comprising the CDR sequences of SEQ ID NO: 19, 29, 32, 34, 36, 37, or 117, or a functionally equivalent CAR thereof.

Embodiment 216. The use of embodiment 214 or 215, wherein the allogeneic CD19-CAR-T cells comprise a CAR comprising the scFv sequence of SEQ ID NO: 19, 29, 32, 34, 36, 37, or 117, or a functionally equivalent CAR thereof.

Embodiment 217. The use of any one of embodiments 214-216, wherein the allogeneic CD19-CAR-T cells comprise a CAR comprising the sequence of 32, 34, 36, or 117, or a functionally equivalent CAR thereof.

Embodiment 218. The use of embodiment 214, wherein the prior treatment comprises axicabtagene ciloleucel, lisocabtagene maraleucel, brexucabtagene autoleucel, or tisagenlecleucel, or a functionally equivalent treatment thereof.

Embodiment 219. The use of any one of embodiments 162-218, wherein the prior treatment is a failed prior treatment.

Embodiment 220. The use of embodiment 219, wherein the failed prior treatment is characterized by one or more of: (a) a plateau or increase in one or more symptom of the disease, (b) a plateau or a worsening of the extent or state of the disease, (c) a plateau or a worsening of disease progression, (d) an attenuated response to therapy, and (e) disease recurrence.

Embodiment 221. The use of any one of embodiments 162-220, wherein the antigen binding domain of the one or more CARs binds to one or more antigens associated with the disease or the disorder.

Embodiment 222. The use of any one of embodiments 162-221, wherein the disease or disorder is cancer.

Embodiment 223. The use of embodiment 222, wherein the cancer is a lymphoma, such as a B cell lymphoma.

Embodiment 224. The use of any one of embodiments 162-223, wherein the patient is treated with an immunodepleting therapy prior to administering the engineered CAR-T cells.

Embodiment 225. The use of any one of embodiments 162-224, wherein the immunodepleting therapy administered prior to administering the engineered CAR-T cells is lower than the immunodepleting therapy administered to the patient prior to the prior treatment.

Embodiment 226. The use of embodiment 225, wherein the immunodepleting therapy comprises fewer doses than the immunodepleting therapy administered to the patient prior to the prior treatment.

Embodiment 227. The use of embodiment 225 or 226, wherein the immunodepleting therapy comprises a reduced amount of immunodepleting agent than the immunodepleting therapy administered to the patient prior to the prior treatment.

Embodiment 228. The use of any one of embodiments 162-227, wherein the immunodepleting therapy comprises administration of fludarabine and/or cyclophosphamide.

Embodiment 229. The use of any one of embodiments 162-228, wherein the immunodepleting therapy comprises IV infusion of about 1-50 mg/m2 of fludarabine for about 1-7 days.

Embodiment 230. The use of embodiment 229, wherein the immunodepleting therapy comprises IV infusion of about 1, about 5, about 10, about 20, about 30, about 40, or about 50 mg/m2 of fludarabine for about 1, about 2, about 3, about 4, about 5, about 6, or about 7 days.

Embodiment 231. The use of embodiment 229 or 230, wherein the immunodepleting therapy comprises IV infusion of about 30 mg/m2 of fludarabine for about 5 days.

Embodiment 232. The use of embodiment 229 or 230, wherein the immunodepleting therapy comprises IV infusion of about 30 mg/m2 of fludarabine for about 3 days.

Embodiment 233. The use of any one of embodiments 162-232, wherein the immunodepleting therapy comprises IV infusion of about 100-1000 mg/m2 of cyclophosphamide for about 1-7 days.

Embodiment 234. The use of embodiment 233, wherein the immunodepleting therapy comprises IV infusion of about 100, about 200, about 300, about 400, about 500, about 600, about 700, about 800, about 900, or about 1000 mg/m2 of cyclophosphamide for about 1, about 2, about 3, about 4, about 5, about 6, or about 7 days.

Embodiment 235. The use of embodiment 234, wherein the immunodepleting therapy comprises IV infusion of about 500 mg/m2 or more of cyclophosphamide for about 5 days.

Embodiment 236. The use of embodiment 234 or 235, wherein the immunodepleting therapy further comprises IV infusion of about 3 mg, about 10 mg, or about 30 mg of alemtuzumab for about 1, about 2, about 3, about 4, about 5, about 6, or about 7 days.

Embodiment 237. The use of embodiment 234, wherein the immunodepleting therapy comprises IV infusion of about 500 mg/m2 of cyclophosphamide for about 3 days.

Embodiment 238. The use of any one of embodiments 162-237, wherein the administration is selected from the group consisting of intravenous injection, intramuscular injection, intravascular injection, and transplantation.

Embodiment 239. The use of any one of embodiments 162-238, wherein at least about 40×104 engineered CAR-T cells are administered to the patient.

Embodiment 240. The use of any one of embodiments 162-239, wherein at least about 40×104 engineered CAR-T cells are administered to the patient.

Embodiment 241. The use of any one of embodiments 162-240, wherein up to about 8.0×108 engineered CAR-T cells are administered to the patient, optionally wherein up to about 6.0×108 engineered CAR-T cells are administered to the patient, optionally wherein about 1.0×106 to about 2.5×108 engineered CAR-T cells are administered to the patient or wherein about 2.0×106 to about 2.0×108 engineered CAR-T cells are administered to the patient.

Embodiment 242. The use of any one of embodiments 162-241, wherein up to about 6.0×108 engineered CAR-T cells are administered to the patient in about 1-3 doses, optionally wherein (a) about 0.6×106 to about 6.0×108 engineered CAR-T cells are administered to the patient in about 1-3 doses, (b) about 0.2×106 to about 5.0×106 engineered CAR-T cells per kg of the patient's body weight are administered to the patient in about 1-3 doses, if the patient has a body weight of 50 kg or less, (c) about 0.1×108 to about 2.5×108 engineered CAR-T cells are administered to the patient in about 1-3 doses, if the patient has a body weight greater than 50 kg, or (d) about 2.0×106 engineered CAR-T cells per kg of the patient's body weight and up to about 2.0×108 engineered CAR-T cells are administered to the patient in about 1-3 doses.

Embodiment 243. The use of any one of embodiments 162-242, wherein about 40×106 to about 200×106 engineered CAR-T cells are administered to the patient, optionally wherein (a) about 40×106 to about 60×106 engineered CAR-T cells are administered to the patient, (b) about 60×106 to about 80×106 engineered CAR-T cells are administered to the patient, (c) about 80×106 to about 100×106 engineered CAR-T cells are administered to the patient, (d) about 100×106 to about 120×106 engineered CAR-T cells are administered to the patient, (e) about 120×106 to about 140×106 engineered CAR-T cells are administered to the patient, (f) about 140×106 to about 160×106 engineered CAR-T cells are administered to the patient, (g) about 160×106 to about 180×106 engineered CAR-T cells are administered to the patient, or (h) about 180×106 to about 200×106 engineered CAR-T cells are administered to the patient.

Embodiment 244. The use of any one of embodiments 162-243, wherein about 60×106 to about 120×106 engineered CAR-T cells are administered to the patient, optionally wherein (a) about 60×106 to about 80×106 engineered CAR-T cells are administered to the patient, (b) about 80×106 to about 100×106 engineered CAR-T cells are administered to the patient, or (c) about 100×106 to about 120×106 engineered CAR-T cells are administered to the patient.

Embodiment 245. The use of any one of embodiments 162-244, wherein about 120×106 to about 200×106 engineered CAR-T cells are administered to the patient, (a) about 120×106 to about 140×106 engineered CAR-T cells are administered to the patient, (b) about 140×106 to about 160×106 engineered CAR-T cells are administered to the patient, (c) about 160×106 to about 180×106 engineered CAR-T cells are administered to the patient, or (d) about 180×106 to about 200×106 engineered CAR-T cells are administered to the patient.

Embodiment 246. The use of any one of embodiments 162-245, wherein the prior treatment comprises an autologous or allogeneic cell-based therapy, and wherein fewer or a lower number of engineered CAR-T cells are administered to the patient than were included in the prior therapy.

Embodiment 247. The use of any one of embodiments 162-246, further comprising administering a second, third, fourth, fifth, or sixth dose of the engineered CAR-T cells to the patient.

Embodiment 248. The use of embodiment 247, wherein the patient is not treated with an immunodepleting therapy prior to the second, third, fourth, fifth, and/or sixth administration of the engineered CAR-T cells.

Embodiment 249. The use of embodiment 247, wherein the patient is treated with an immunodepleting therapy prior to the second, third, fourth, fifth, and/or sixth administration of the engineered CAR-T cells.

Embodiment 250. The use of embodiment 249, wherein the immunodepleting therapy that is administered prior to the second, third, fourth, fifth, and/or sixth administration of the engineered CAR-T cells is independently selected from administration of fludarabine and/or cyclophosphamide, wherein the administration of fludarabine comprises IV infusion of about 1-50 mg/m2 of fludarabine for about 1-7 days, and the administration of cyclophosphamide comprises IV infusion of about 100-1000 mg/m2 of cyclophosphamide for about 1-7 days.

Embodiment 251. The use of any one of embodiments 162-250, wherein the engineered CAR-T cells are propagated from a primary T cell or a progeny thereof, or are derived from a T cell differentiated from an iPSC or a progeny thereof.

Embodiment 252. The use of any one of embodiments 162-251, wherein the engineered CAR-T cells are differentiated cells derived from an induced pluripotent stem cell or a progeny thereof.

Embodiment 253. The use of embodiment 252, wherein the differentiated cells are a T cells or NK cells.

Embodiment 254. The use of any one of embodiments 162-251, wherein the engineered CAR-T cells are a progeny of primary immune cells.

Embodiment 255. The use of embodiment 254, wherein the progeny of primary immune cells are T cells or NK cells.

Embodiment 256. The use of any one of embodiments 162-255, wherein the wild type cell or the control cell is a starting material.

Embodiment 257. The use of any one of embodiments 162-256, wherein the engineered CAR-T cells are CAR+ T cells that comprise any one selected from the group consisting of a bulk population of CAR+ T cells, CD4+ CAR+ T cells, CD8+ CAR+ T cells, and a combination thereof.

Embodiment 258. The use of embodiment 257, wherein the CD4+ CAR+ T cells and CD8+ CAR+ T cells are administered concomitantly or sequentially.

Embodiment 259. The use of embodiment 258, wherein the CD4+ CAR+ T cells are administered prior to administration of the CD8+ CAR+ T cells, or wherein the CD8+ CAR+ T cells are administered prior to administration of the CD4+ CAR+ T cells.

Embodiment 260. The use of embodiment 257, wherein the bulk CAR+ T cells and CD8+ CAR+ T cells are administered concomitantly or sequentially.

Embodiment 261. The use of embodiment 260, wherein the bulk CAR+ T cells are administered prior to administration of the CD8+ CAR+ T cells, or wherein the CD8+ CAR+ T cells are administered prior to administration of the bulk CAR+ T cells.

Embodiment 262. The use of embodiment 257, wherein the CD4+ CAR+ T cells and bulk CAR+ T cells are administered concomitantly or sequentially.

Embodiment 263. The use of embodiment 262, wherein the CD4+ CAR+ T cells are administered prior to administration of the bulk CAR+ T cells, or wherein the bulk CAR+ T cells are administered prior to administration of the CD4+ CAR+ T cells.

Embodiment 264. The use of any one of embodiments 162-263, wherein the engineered CAR-T cells comprise reduced expression of B2M and/or CIITA relative to an unaltered control cell.

Embodiment 265. The use of embodiment 264, wherein the engineered CAR-T cells do not express B2M and/or CIITA.

Embodiment 266. The use of any one of embodiments 162-265, wherein the engineered CAR-T cells comprise reduced expression of a TCR.

Embodiment 267. The use of embodiment 266, wherein the engineered CAR-T cells comprise reduced expression of TRAC and/or TRBC.

Embodiment 268. The use of embodiment 266 or 267, wherein the engineered CAR-T cells do not express TRAC and/or TRBC.

Embodiment 269. The use of any one of embodiments 162-268, wherein the engineered CAR-T cells comprise reduced expression of HLA class I antigens and/or HLA class II antigens relative to an unaltered control cell.

Embodiment 270. The use of embodiment 269, wherein the engineered CAR-T cells do not express HLA class I antigens, HLA class 11 antigens, and/or do not express TCR-alpha.

Embodiment 271. The use of embodiment 269 or 270, wherein the reduced expression or no expression of HLA class I antigens results from the reduced expression or no expression of B2M, and where in the reduced expression or no expression of HLA class 11 antigens results from the reduced expression or no expression of CIITA.

Embodiment 272. The use of any one of embodiments 162-271, wherein the engineered CAR-T cells are B2Mindel/indel, CIITAindel/indel cell, and/or a TRACindel/indel, and/or TRACindel/indel cells.

Embodiment 273. The use of any one of embodiments 162-272, wherein the engineered CAR-T cells comprise reduced expression of HLA-A, HLA-B, HLA-C, HLA-DP, HLA-DQ, HLA-DR, RHD, ABO, PCDH11Y, and/or NLGN4Y relative to an unaltered control cell.

Embodiment 274. The use of embodiment 273, wherein the engineered CAR-T cells do not express HLA-A, HLA-B, HLA-C, HLA-DP, HLA-DQ, HLA-DR, RHD, ABO, PCDH11Y, and/or NLGN4Y.

Embodiment 275. The use of any one of embodiments 162-274, wherein the reduced expression is by way of gene knock down, optionally wherein the gene knock down is by way of RNA silencing or RNAi, optionally selected from the group consisting of siRNAs, piRNAs, shRNAs, and miRNAs.

Embodiment 276. The use of any one of embodiments 162-275, wherein the reduced expression is by way of gene knock out, optionally wherein the gene knock out is by way of inducing an insertion or a deletion in the gene using a gene editing system, wherein the gene editing system is optionally selected from the group consisting of ZFNs, TALENs, meganucleases, transposases, CRISPR/Cas systems, nickase systems, base editing systems, prime editing systems, and gene writing systems.

Embodiment 277. The use of any one of embodiments 162-276, wherein the one or more tolerogenic factors are selected from the group consisting of CD47, CD24, CD27, CD35, CD46, CD55, CD59, CD200, HLA-C, HLA-E, HLA-E heavy chain, HLA-G, PD-L1, IDO1, CTLA4-Ig, C1-Inhibitor (e.g., CR1), IL-10, IL-35, FasL, CCL21, CCL22, Mfge8, and Serpinb9.

Embodiment 278. The use of embodiment 277, wherein the one or more tolerogenic factors comprise CD47.

Embodiment 279. Use of a population of engineered CAR-T cells for treating a disease or disorder in a patient who has undergone one or more prior treatments for the disease or disorder, wherein the engineered CAR-T cells comprise reduced expression of B2M and TRAC, relative to an unaltered control cell, a first exogenous polynucleotide encoding HLA-E, and a second exogenous polynucleotide encoding one or more CARs, wherein at least one CAR comprises a CD22 antigen binding domain having the CDR sequences from SEQ ID NO: 45, 54, 85, 91, 92, or 93, and wherein the disease or disorder is a cancer.

Embodiment 280. The use of embodiment 279, wherein the HLA-E is a single chain trimer.

Embodiment 281. The use of embodiment 279, wherein the HLA-E is a HLA-E/B2M fusion.

Embodiment 282. Use of a population of engineered CAR-T cells for treating a disease or disorder in a patient who has undergone one or more prior treatments for the disease or disorder, wherein the engineered CAR-T cells comprise reduced expression of B2M and/or CR-1 and TRAC, relative to an unaltered control cell, a first exogenous polynucleotide encoding CD24, and a second exogenous polynucleotide encoding one or more CARs, wherein at least one CAR comprises a CD22 antigen binding domain having the CDR sequences from SEQ ID NO: 45, 54, 85, 91, 92, or 93, and wherein the disease or disorder is a cancer.

Embodiment 283. Use of a population of engineered CAR-T cells for treating a disease or disorder in a patient who has undergone one or more prior treatments for the disease or disorder, wherein the engineered CAR-T cells comprise reduced expression of B2M and/or CD52 and TRAC, relative to an unaltered control cell, optionally a first exogenous polynucleotide encoding a tolerogenic factor, and a second exogenous polynucleotide encoding one or more CARs, wherein at least one CAR comprises a CD22 antigen binding domain having the CDR sequences from SEQ ID NO: 45, 54, 85, 91, 92, or 93, and wherein the disease or disorder is a cancer.

Embodiment 284. Use of a population of engineered CAR-T cells for treating a disease or disorder in a patient who has undergone one or more prior treatments for the disease or disorder, wherein the engineered CAR-T cells comprise reduced expression of B2M and/or CD70 and TRAC, relative to an unaltered control cell, optionally a first exogenous polynucleotide encoding a tolerogenic factor, and a second exogenous polynucleotide encoding one or more CARs, wherein at least one CAR comprises a CD22 antigen binding domain having the CDR sequences from SEQ ID NO: 45, 54, 85, 91, 92, or 93, and wherein the disease or disorder is a cancer.

Embodiment 285. Use of a population of engineered CAR-T cells for treating a disease or disorder in a patient who has undergone one or more prior treatments for the disease or disorder, wherein the engineered CAR-T cells comprise reduced expression of PD-1 and TRAC, relative to an unaltered control cell, optionally a first exogenous polynucleotide encoding a tolerogenic factor, and a second exogenous polynucleotide encoding one or more CARs, wherein at least one CAR comprises a CD22 antigen binding domain having the CDR sequences from SEQ ID NO: 45, 54, 85, 91, 92, or 93, and wherein the disease or disorder is a cancer.

Embodiment 286. The use of any one of embodiments 162-285, wherein the engineered CAR-T cells comprise a third exogenous polynucleotide encoding a CD19-specific CAR.

Embodiment 287. The use of embodiment 286, wherein the CD19-specific CAR comprises a hinge domain of any one of SEQ ID NOs: 9-13, a transmembrane sequence of any one of SEQ ID NOs: 14, 15, and 114, and/or an intracellular costimulatory and/or signaling domain of any one of SEQ ID NOs: 16-18 and 115.

Embodiment 288. The use of any one of embodiments 162-287, wherein the first exogenous polynucleotide, the second exogenous polynucleotide, and/or the third exogenous polynucleotides are carried by a polycistronic vector.

Embodiment 289. The use of any one of embodiments 162-288, wherein the CD22-specific CAR, the one or more tolerogenic factors, and/or the additional CD19-specific CAR are carried by a single polycistronic vector.

Embodiment 290. The use of embodiment 288 or 289, wherein the polycistronic vector is a bicistronic vector.

Embodiment 291. The use of any one of embodiments 162-290, wherein the first, second, and/or third exogenous polynucleotide, and/or the polycistronic vector is inserted into a first, second, and/or third specific locus of at least one allele of the cell.

Embodiment 292. The use of embodiment 291, wherein the first, second, and/or third specific loci are selected from the group consisting of a safe harbor locus, a target locus, an RHD locus, a B2M locus, a CIITA locus, a TRAC locus, and a TRB locus.

Embodiment 293. The use of embodiment 292, wherein the safe harbor locus is selected from the group consisting of a CCR5 locus, a PPP1R12C locus, a CLYBL locus, and a Rosa locus.

Embodiment 294. The use of embodiment 292, wherein the target locus is selected from the group consisting of a CXCR4 locus, an ALB locus, a SHS231 locus, an F3 (CD142) locus, a MICA locus, a MICB locus, a LRP1 (CD91) locus, a HMGB1 locus, an ABO locus, a FUT1 locus, and a KDM5D locus.

Embodiment 295. Use of a population of engineered CAR-T cells for treating a disease or disorder in a patient who has undergone one or more prior treatments for the disease or disorder, wherein the engineered CAR-T cells comprise reduced expression of B2M, CIITA, and TRAC, relative to an unaltered control cell, a first exogenous polynucleotide encoding CD47, and a second exogenous polynucleotide encoding a CD22 CAR comprising a sequence having at least 90% sequence homology to the sequence set forth in SEQ ID NO: 91, wherein the first exogenous polynucleotide and the second exogenous polynucleotide are inserted by a bicistronic vector, and wherein the disease or disorder is a cancer.

Embodiment 296. Use of a population of engineered CAR-T cells for treating a disease or disorder in a patient who has undergone one or more prior treatments for the disease or disorder, wherein the engineered CAR-T cells comprise reduced expression of B2M, CIITA, and TRAC, relative to an unaltered control cell, a first exogenous polynucleotide encoding CD47, and a second exogenous polynucleotide encoding a CD22 CAR comprising the sequence set forth in SEQ ID NO: 91, wherein the first exogenous polynucleotide and the second exogenous polynucleotide are inserted by a bicistronic vector, and wherein the disease or disorder is a cancer.

Embodiment 297. Use of a population of engineered CAR-T cells for treating a disease or disorder in a patient who has undergone one or more prior treatments for the disease or disorder, wherein the engineered CAR-T cells comprise reduced expression of B2M, CIITA, and TRAC, relative to an unaltered control cell, a first exogenous polynucleotide encoding CD47, a second exogenous polynucleotide encoding a CD22 CAR comprising a sequence having at least 90% sequence homology to the sequence set forth in SEQ ID NO: 91, and a third exogenous polynucleotide encoding a CD19 CAR comprising a sequence having at least 90% sequence homology to the sequence set forth in SEQ ID NO: 117 and wherein the disease or disorder is a cancer.

Embodiment 298. Use of a population of engineered CAR-T cells for treating a disease or disorder in a patient who has undergone one or more prior treatments for the disease or disorder, wherein the engineered CAR-T cells comprise reduced expression of B2M, CIITA, and TRAC, relative to an unaltered control cell, a first exogenous polynucleotide encoding CD47, a second exogenous polynucleotide encoding a CD22 CAR comprising the sequence set forth in SEQ ID NO: 91, and a third exogenous polynucleotide encoding a CD19 CAR comprising a sequence having at least 90% sequence homology to the sequence set forth in SEQ ID NO: 117 and wherein the disease or disorder is a cancer.

Embodiment 299. The use of any one of embodiments 162-298, wherein the first exogenous polynucleotide, the second exogenous polynucleotide, and/or the third exogenous polynucleotides are carried by a polycistronic vector.

Embodiment 300. The use of embodiment 299, wherein the polycistronic vector is a bicistronic vector.

Embodiment 301. The use of any one of embodiments 162-300, wherein the first, second, and/or third exogenous polynucleotide or the polycistronic vector is introduced into the engineered CAR-T cells using CRISPR/Cas gene editing.

Embodiment 302. The use of embodiment 301, wherein the CRISPR/Cas gene editing is carried out ex vivo from a donor patient.

Embodiment 303. The use of any one of embodiments 162-302, wherein the first, second, and/or third exogenous polynucleotide, and/or the polycistronic vector is inserted into at least one allele of the engineered CAR-T cell using viral transduction.

Embodiment 304. The use of embodiment 303, wherein the viral transduction includes a lentivirus based viral vector.

Embodiment 305. The use of embodiment 304, wherein the lentivirus based viral vector is a pseudotyped, self-inactivating lentiviral vector that carries the first, second, and/or third exogenous polynucleotide, and/or the polycistronic vector.

Embodiment 306. The use of any one of embodiments 162-305, wherein the lentivirus based viral vector is a pseudotyped, self-inactivating lentiviral vector that carries the first and second exogenous polynucleotides.

Embodiment 307. The use of any one of embodiments 162-306, wherein the lentiviral vector comprises the first exogenous polynucleotide followed by the second exogenous polynucleotide.

Embodiment 308. The use of any one of embodiments 162-307, wherein the lentiviral vector comprises the second exogenous polynucleotide followed by the first exogenous polynucleotide.

Embodiment 309. The use of embodiment 304-308, wherein the lentivirus based viral vector is a self-inactivating lentiviral vector pseudotyped with a vesicular stomatitis VSV-G envelope and carries the first, second, and/or third exogenous polynucleotide, and/or the polycistronic vector.

Embodiment 310. The use of any one of embodiments 304-309, wherein the CD22-specific CAR and/or the CD19-specific CAR are inserted using one or more lentiviral vectors, and the CD47 is inserted using another lentiviral vector.

Embodiment 311. The use of any one of embodiments 304-309, wherein the CD22-specific CAR and/or the CD19-specific CAR are inserted using one or more lentiviral vectors, and the CD47 is inserted using a locus-specific insertion method, optionally a CRISPR/Cas or a TALEN method.

Embodiment 312. The use of any one of embodiments 304-309, wherein the CD22-specific CAR and/or the CD19-specific CAR are inserted using a locus-specific insertion method, optionally a CRISPR/Cas or a TALEN method, and the CD47 is inserted using a lentiviral vector.

Embodiment 313. The use of any one of embodiments 304-309, wherein the CD22-specific CAR and/or the CD19-specific CAR and the CD47 are inserted using one or more lentiviral vectors.

Embodiment 314. The use of any one of embodiments 304-309, wherein the CD22-specific CAR and/or the CD19-specific CAR and the CD47 are inserted using a locus-specific insertion method, optionally a CRISPR/Cas or a TALEN method.

Embodiment 315. The use of any one of embodiments 162-314, wherein the engineered CAR-T cells evade NK cell mediated cytotoxicity upon administration to the patient.

Embodiment 316. The use of any one of embodiments 162-315, wherein the engineered CAR-T cells are protected from cell lysis by mature NK cells upon administration to the patient.

Embodiment 317. The use of any one of embodiments 162-316, wherein the engineered CAR-T cells evade macrophage-mediated cytotoxicity, optionally wherein the macrophage-mediated cytotoxicity involves phagocytosis and/or reactive oxygen species.

Embodiment 318. The use of any one of embodiments 162-317, wherein the engineered CAR-T cells do not induce an immune response to the cell upon administration to the patient.

Embodiment 319. The use of any one of embodiments 162-318, wherein the engineered CAR-T cells persist in the patient for at least 4 weeks, at least 2 months, at least 3 months, at least 4 months, at least 5 months, at least 6 months, at least 7 months, at least 8 months, at least 9 months, at least 10 months, at least 11 months, at least 12 months, or longer.

Embodiment 320. The use of any one of embodiments 162-319, wherein the prior treatment comprises an autologous or allogeneic cell-based therapy, and wherein the engineered CAR-T cells persist in the patient for longer than the cells of the prior therapy.

Embodiment 321. The use of any one of embodiments 162-320, wherein the therapeutic effect of the engineered CAR-T cells lasts for a duration of at least 4 weeks, at least 2 months, at least 3 months, at least 4 months, at least 5 months, at least 6 months, at least 7 months, at least 8 months, at least 9 months, at least 10 months, at least 11 months, at least 12 months, or longer.

Embodiment 322. The use of any one of embodiments 162-321, wherein the therapeutic effect of the engineered CAR-T cells lasts for longer than that of the prior therapy.

1. A method of treating a disease or disorder in a patient, the method comprising administering a therapeutic agent directed to a first therapeutic target.

2. The method of Item 1, wherein the therapeutic agent is further directed to a second therapeutic target, wherein the first therapeutic target and the second therapeutic target are different.

3. The method of Item 1, wherein the patient has previously been administered one or more targeted therapies directed to a second therapeutic target, wherein the first therapeutic target and the second therapeutic target are different.

4. The method of any one of Items 1-3, wherein the patient has not previously been administered a targeted therapy for the treatment of the disease or disorder.

5. The method of Item 1, wherein the patient has previously been administered one or more targeted therapies directed to a second therapeutic target, wherein the therapeutic agent is further directed to the second therapeutic target, and wherein the first therapeutic target and the second therapeutic target are different.

6. The method of any one of Items 1-5, wherein the patient has not previously received a therapy directed to the first therapeutic target.

7. The method of any one of Items 2-6, wherein the patient has not previously received a therapy directed to the second therapeutic target.

8. The method of Item 1, wherein the patient is at risk of antigen evasion, and wherein the therapeutic agent is directed to the first therapeutic target and a second therapeutic target, wherein the first therapeutic target and the second therapeutic target are different therapeutic targets.

9. The method of Item 1, wherein the patient has previously been administered one or more targeted therapies directed to a second therapeutic target, wherein the therapeutic agent comprises a first population of engineered CAR-T cells, wherein the engineered CAR-T cells of the first population comprise one or more chimeric antigen receptors (CARs), wherein at least one CAR is directed to the first therapeutic target, and wherein the first therapeutic target and the second therapeutic target are different.

10. The method of Item 1, wherein the disease or disorder is characterized by antigen evasion, wherein the patient has previously been administered one or more targeted therapies directed to a second therapeutic target, wherein the therapeutic agent comprises a first population of engineered CAR-T cells, wherein the engineered CAR-T cells of the first population comprise one or more chimeric antigen receptors (CARs), wherein at least one CAR is directed to the first therapeutic target, and wherein the first therapeutic target and the second therapeutic target are different.

11. The method of any one of Items 2-7, wherein the patient is at risk of antigen evasion, wherein the therapeutic agent comprises a first population of engineered CAR-T cells, wherein the engineered CAR-T cells of the first population comprise one or more chimeric antigen receptors (CARs), wherein at least one CAR is directed to the first therapeutic target, and wherein the first therapeutic target and the second therapeutic target are different.

12. The method of Item 1, wherein the patient is at risk of antigen evasion, wherein the patient has previously been administered one or more targeted therapies directed to a second therapeutic target, wherein the therapeutic agent comprises a population of engineered CAR-T cells, wherein the engineered CAR-T cells of the population comprise one or more chimeric antigen receptors (CARs), wherein at least one CAR is directed to the first therapeutic target, and wherein the first therapeutic target and the second therapeutic target are different.

13. The method of any one of Items 1-12, wherein the first therapeutic target is a first antigen.

14. The method of Item 13, wherein the first antigen is an antigen associated with the disease or the disorder.

15. The method of Item 13 or 14, wherein the first antigen is an antigen present on the surface of a B cell.

16. The method of Item 15, wherein the B cell is a malignant B cell.

17. The method of any one of Items 13-16, wherein the first antigen is CD22, CD20, CD19, BCMA, GPRC5D, CD38, CD70, CD79b, HER2, IL13Ra2, MUC1, or a variant thereof.

18. The method of any one of Items 13-17, wherein the first antigen is CD22, CD20, CD19, BCMA, GPRC5D, CD38, CD70, CD79b, HER2, IL13Ra2, or MUC1.

19. The method of any one of Items 2-18, wherein the second therapeutic target is a second antigen.

20. The method of Item 19, wherein the second antigen is an antigen associated with the disease or the disorder.

21. The method of Item 19 or 20, wherein the second antigen is an antigen present on the surface of a B cell.

22. The method of Item 21, wherein the B cell is a malignant B cell.

23. The method of any one of Items 19-22, wherein the second antigen is CD22, CD20, CD19, BCMA, GPRC5D, CD38, CD70, CD79b, HER2, IL13Ra2, MUC1, or a variant thereof.

24. The method of any one of Items 19-23, wherein the second antigen is CD22, CD20, CD19, BCMA, GPRC5D, CD38, CD70, CD79b, HER2, IL13Ra2, or MUC1.

25. The method of any one of Items 19-24, wherein the second antigen is CD22, CD20, or CD19.

26. The method of any one of Items 2-25, wherein the therapeutic agent comprises a first immunotherapeutic agent.

27. The method of any one of Items 2-26, wherein the therapeutic agent comprises a first population of engineered cells.

28. The method of Item 27, wherein the first population of engineered cells comprises engineered cells directed to the first therapeutic target.

29. The method of Item 27 or 28, wherein the first population of engineered cells comprises engineered cells that comprise a first immunotherapeutic agent.

30. The method of Item 26 or 29, wherein the first immunotherapeutic agent comprises a first antigen binding domain.

31. The method of any one of Items 26, 29, and 30, wherein the first immunotherapeutic agent comprises an antibody, a Fab, an scFV, an scFV-Fc, an scFV zipper, a diabody, a minibody, a CAR, a CAAR, a CAAR-T cell, a BAR, or a BAR-T cell.

32. The method of any one of Items 27-31, wherein the first population of engineered cells is a first population of engineered CAR-T cells.

33. The method of Item 32, wherein the first population of engineered CAR-T cells comprise one or more chimeric antigen receptors (CARs), wherein at least one CAR of the first population of engineered CAR-T cells,
(i) is directed to the first therapeutic target, and
(ii) comprises the first antigen binding domain.

34. The method of Item 32 or 33, wherein at least one CAR of the first population of engineered CAR-T cells,
(i) is directed to the second therapeutic target and
(ii) comprises a second antigen binding domain.

35. The method of any one of Items 26-34, wherein the therapeutic agent further comprises a second immunotherapeutic agent.

36. The method of any one of Items 26-35, wherein the therapeutic agent further comprises a second population of engineered cells.

37. The method of Item 36, wherein the second population of engineered cells comprises engineered cells directed to the second therapeutic target.

38. The method of Item 36 or 37, wherein the second population of engineered cells comprises engineered cells that comprise a second immunotherapeutic agent.

39. The method of Item 38, wherein the second immunotherapeutic agent comprises a second antigen binding domain.

40. The method of Item 38 or 39, wherein the second immunotherapeutic agent comprises an antibody, a Fab, an scFV, an scFV-Fc, an scFV zipper, a diabody, a minibody, a CAR, a CAAR, a CAAR-T cell, a BAR, or a BAR-T cell.

41. The method of any one of Items 36-40, wherein the second population of engineered cells is a second population of engineered CAR-T cell.

42. The method of any one of Items 36-41, wherein the therapeutic agent comprises a second population of engineered CAR-T cells, wherein the second population of engineered CAR-T cells comprise one or more chimeric antigen receptors (CARs), wherein at least one CAR comprises a second antigen binding domain.

43. The method of any one of Items 1-26, wherein the therapeutic agent comprises one or more populations of engineered CAR-T cells.

44. The method of Item 43, wherein the therapeutic agent comprises a first population of engineered CAR-T cells and a second population of engineered CAR-T cells, wherein the first population of engineered CAR-T cells comprise one or more chimeric antigen receptors (CARs), wherein at least one CAR of the first population of engineered CAR-T cells (i) is directed to the first therapeutic target, and
(ii) comprises the first antigen binding domain, and wherein the second population of engineered CAR-T cells comprise one or more chimeric antigen receptors (CARs), wherein at least one CAR of the second population of engineered CAR-T cell
(i) is directed to the second therapeutic target, and
(ii) comprises the second antigen binding domain.

45. The method of any one of Items 1-26, wherein the therapeutic agent comprises a first population of engineered CAR-T cells and a second population of engineered CAR-T cells, wherein the first population of engineered CAR-T cells comprise one or more chimeric antigen receptors (CARs), wherein at least one CAR of the first population of engineered CAR-T cells,
(i) is directed to the first therapeutic target, and
(ii) comprises a first antigen binding domain, wherein the second population of engineered CAR-T cells comprise two or more chimeric antigen receptors (CARs), wherein at least one CAR of the second population of engineered CAR-T cells,
(i) is directed to the first therapeutic target, and
(ii) comprises a first antigen binding domain, and wherein at least one CAR of the second population of engineered CAR-T cells,
(i) is directed to the second therapeutic target and
(ii) comprises a second antigen binding domain.

46. The method of any one of Items 1-26, wherein the therapeutic agent comprises a first population of engineered CAR-T cells, a second population of engineered CAR-T cells, and a third population of engineered CAR-T cells, wherein the first population of engineered CAR-T cells comprise one or more chimeric antigen receptors (CARs), wherein at least one CAR of the first population of engineered CAR-T cells,
(i) is directed to the first therapeutic target, and
(ii) comprises a first antigen binding domain, wherein the second population of engineered CAR-T cells comprise one or more chimeric antigen receptors (CARs), wherein at least one CAR of the second population of engineered CAR-T cells
(i) is directed to the second therapeutic target, and
(ii) comprises a second antigen binding domain, and wherein the third population of engineered CAR-T cells comprise two or more chimeric antigen receptors (CARs), wherein at least one CAR of the third population of engineered CAR-T cells
(i) is directed to the first therapeutic target, and
(ii) comprises a first antigen binding domain, and wherein at least one CAR of the third population of engineered CAR-T cells
(i) is directed to the second therapeutic target and
(ii) comprises a second antigen binding domain.

47. The method of any one of Items 30-46, wherein the first antigen binding domain is capable of binding to CD22 or a variant thereof.

48. The method of any one of Items 30-47, wherein the first antigen binding domain is capable of binding to CD22.

49. The method of any one of Items 30-48, wherein the first antigen binding domain comprises a heavy chain complementarity determining region 1 (HCDR1) comprising an amino acid sequence according to SEQ ID NO: 47, a heavy chain complementarity determining region 2 (HCDR2) comprising an amino acid sequence according to SEQ ID NO: 48, and a heavy chain complementarity determining region 3 (HCDR3) comprising an amino acid sequence according to SEQ ID NO: 49.

50. The method of any one of Items 30-49, wherein the first antigen binding domain comprises a light chain complementarity determining region 1 (LCDR1) comprising an amino acid sequence according to SEQ ID NO: 51, a light chain complementarity determining region 2 (LCDR2) comprising an amino acid sequence according to SEQ ID NO: 52, and a light chain complementarity determining region 3 (LCDR3) comprising an amino acid sequence according to SEQ ID NO: 53.

51. The method of any one of Items 30-48, wherein the first antigen binding domain comprises a heavy chain variable domain (VH) comprising an amino acid sequence according to SEQ ID NO: 47, an amino acid sequence according to SEQ ID NO: 48, and an amino acid sequence according to SEQ ID NO: 49 arranged non-contiguously from N-terminus to C-terminus.

52. The method of any one of Items 30-50, wherein the first antigen binding domain comprises a heavy chain variable domain (VH) comprising an amino acid sequence that is at least 80%, at least 85%, at least 90%, at least 95%, at least 96%, at least 97%, at least 98%, at least 99%, or 100% identical to an amino acid sequence according to SEQ ID NO: 46.

53. The method of any one of Items 30-48 and 51, wherein the first antigen binding domain comprises a light chain variable domain (VL) comprising an amino acid sequence according to SEQ ID NO: 51, an amino acid sequence according to SEQ ID NO: 52, and an amino acid sequence according to SEQ ID NO: 53 arranged non-contiguously from N-terminus to C-terminus.

54. The method of any one of Items 30-50 and 52, wherein the first antigen binding domain comprises a light chain variable domain (VL) comprising an amino acid sequence that is at least 80%, at least 85%, at least 90%, at least 95%, at least 96%, at least 97%, at least 98%, at least 99%, or 100% identical to an amino acid sequence according to SEQ ID NO: 50.

55. The method of any one of Items 30-48 wherein the first antigen binding domain comprises an HCDR1 according to SEQ ID NO: 56, an HCDR2 according to SEQ ID NO: 57, and an HCDR3 according to SEQ ID NO: 58.

56. The method of any one of Items 30-48, and 55, wherein the first antigen binding domain comprises an LCDR1 according to SEQ ID NO: 60, an LCDR2 according to SEQ ID NO: 61, and an LCDR3 according to SEQ ID NO: 62.

57. The method of any one of Items 30-48, wherein the first antigen binding domain comprises a heavy chain variable domain (VH) comprising an amino acid sequence according to SEQ ID NO: 56, an amino acid sequence according to SEQ ID NO: 57, and an amino acid sequence according to SEQ ID NO: 58 arranged non-contiguously from N-terminus to C-terminus.

58. The method of any one of Items 30-48, 55, and 56, wherein the first antigen binding domain comprises a heavy chain variable domain (VH) comprising an amino acid sequence that is at least 80%, at least 85%, at least 90%, at least 95%, at least 96%, at least 97%, at least 98%, at least 99%, or 100% identical to an amino acid sequence according to SEQ ID NO: 55.

59. The method of any one of Items 30-48 and 57, wherein the first antigen binding domain a light chain variable domain (VL) comprising comprises an amino acid sequence according to SEQ ID NO: 60, an amino acid sequence according to SEQ ID NO: 61, and an amino acid sequence according to SEQ ID NO: 62 arranged non-contiguously from N-terminus to C-terminus.

60. The method of any one of Items 30-48, 55, 56, and 58, wherein the first antigen binding domain comprises a light chain variable domain (VL) comprising an amino acid sequence that is at least 80% identical, at least 80%, at least 85%, at least 90%, at least 95%, at least 96%, at least 97%, at least 98%, at least 99%, or 100% identical to an amino acid sequence according to SEQ ID NO: 59.

61. The method of any one of Items 30-48, wherein the first antigen binding domain is capable of binding to CD19 or a variant thereof.

62. The method of any one of Items 30-48, and 61, wherein the first antigen binding domain is capable of binding to CD19.

63. The method of any one of Items 30-48, 61, and 62, wherein the first antigen binding domain comprises an HCDR1 according to SEQ ID NO: 26, an HCDR2 according to SEQ ID NO: 27, and an HCDR3 according to SEQ ID NO: 28.

64. The method of any one of Items 30-48, and 61-63, wherein the first antigen binding domain comprises an LCDR1 according to SEQ ID NO: 21, an LCDR2 according to SEQ ID NO: 22, and an LCDR3 according to SEQ ID NO: 23.

65. The method of any one of Items 30-48, 61 and 62, wherein the first antigen binding domain comprises a heavy chain variable domain (VH) comprising an amino acid sequence according to SEQ ID NO: 26, an amino acid sequence according to SEQ ID NO: 27, and an amino acid sequence according to SEQ ID NO: 28 arranged non-contiguously from N-terminus to C-terminus.

66. The method of any one of Items 30-48, and 61-64, wherein the first antigen binding domain comprises a heavy chain variable domain (VH) comprising an amino acid sequence that is at least 80%, at least 85%, at least 90%, at least 95%, at least 96%, at least 97%, at least 98%, at least 99%, or 100% identical to an amino acid sequence according to SEQ ID NO: 25.

67. The method of any one of Items 30-48, 61, 62 and 65, wherein the first antigen binding domain comprises a light chain variable domain (VL) comprising an amino acid sequence according to SEQ ID NO: 21, an amino acid sequence according to SEQ ID NO: 22, and an amino acid sequence according to SEQ ID NO: 23 arranged non-contiguously from N-terminus to C-terminus.

68. The method of any one of Items 30-48, 61-64 and 66 wherein the first antigen binding domain comprises a light chain variable domain (VL) comprising an amino acid sequence that is at least 80%, at least 85%, at least 90%, at least 95%, at least 96%, at least 97%, at least 98%, at least 99%, or 100% identical to an amino acid sequence according to SEQ ID NO: 20.

69. The method of any one of Items 30-48, wherein the first antigen binding domain is capable of binding to CD20 or a variant thereof.

70. The method of any one of Items 30-48, and 69, wherein the first antigen binding domain is capable of binding to CD20.

71. The method of any one of Items 30-48, 69 and 70, wherein the first antigen binding domain comprises an HCDR1 according to SEQ ID NO: 43, an HCDR2 according to SEQ ID NO: 44, and an HCDR3 according to SEQ ID NO: 107.

72. The method of any one of Items 30-48, and 69-71, wherein the first antigen binding domain comprises an LCDR1 according to SEQ ID NO: 39, an LCDR2 according to SEQ ID NO: 40, and an LCDR3 according to SEQ ID NO: 41.

73. The method of any one of Items 30-48, 69 and 70, wherein the first antigen binding domain comprises a heavy chain variable domain (VH) comprising an amino acid sequence according to SEQ ID NO: 43, an amino acid sequence according to SEQ ID NO: 44, and an amino acid sequence according to SEQ ID NO: 107 arranged non-contiguously from N-terminus to C-terminus.

74. The method of any one of Item s 30-89, and 69-72, wherein the first antigen binding domain comprises a heavy chain variable domain (VH) comprising an amino acid sequence that is at least 80%, at least 85%, at least 90%, at least 95%, at least 96%, at least 97%, at least 98%, at least 99%, or 100% identical to an amino acid sequence according to SEQ ID NO: 42.

75. The method of any one of Items 30-48, 69, 70 and 73, wherein the first antigen binding domain comprises a light chain variable domain (VL) comprising an amino acid sequence according to SEQ ID NO: 39, an amino acid sequence according to SEQ ID NO: 40, and an amino acid sequence according to SEQ ID NO: 41 arranged non-contiguously from N-terminus to C-terminus.

76. The method of any one of Items 30-48, 69-72 and 74, wherein the first antigen binding domain comprises a light chain variable domain (VL) comprising an amino acid sequence that is at least 80%, at least 85%, at least 90%, at least 95%, at least 96%, at least 97%, at least 98%, at least 99%, or 100% identical to an amino acid sequence according to SEQ ID NO: 38.

77. The method of any one of Items 39-42, wherein the second antigen binding domain is capable of binding CD19 or a variant thereof.

78. The method of any one of Items 39-42 and 77, wherein the second antigen binding domain is capable of binding CD19.

79. The method of any one of Items 39-42, 77 and 78, wherein the second antigen binding domain comprises an HCDR1 according to SEQ ID NO: 26, an HCDR2 according to SEQ ID NO: 27, and an HCDR3 according to SEQ ID NO: 28.

80. The method of any one of Items 39-42, and 77-79, wherein the second antigen binding domain comprises an LCDR1 according to SEQ ID NO: 21, an LCDR2 according to SEQ ID NO: 22, and an LCDR3 according to SEQ ID NO: 23.

81. The method of any one of Items 39-42, 77 and 78, wherein the first antigen binding domain comprises a heavy chain variable domain (VH) comprising an amino acid sequence according to SEQ ID NO: 27, an amino acid sequence according to SEQ ID NO: 28, and an amino acid sequence according to SEQ ID NO: 29 arranged non-contiguously from N-terminus to C-terminus.

82. The method of any one of Items 39-42 and 77-80, wherein the second antigen binding domain comprises a heavy chain variable domain (VH) comprising an amino acid sequence that is at least 80%, at least 85%, at least 90%, at least 95%, at least 96%, at least 97%, at least 98%, at least 99%, or 100% identical to an amino acid sequence according to SEQ ID NO: 25.

83. The method of any one of Items 39-42, 77, 78, and 81 wherein the first antigen binding domain comprises a light chain variable domain (VL) comprising an amino acid sequence according to SEQ ID NO: 21, an amino acid sequence according to SEQ ID NO: 22, and an amino acid sequence according to SEQ ID NO: 23 arranged non-contiguously from N-terminus to C-terminus.

84. The method of any one of Items 39-42, 77-80 and 82 wherein the second antigen binding domain comprises a light chain variable domain (VL) comprising an amino acid sequence that is at least 80%, at least 85%, at least 90%, at least 95%, at least 96%, at least 97%, at least 98%, at least 99%, or 100% identical to an amino acid sequence according to SEQ ID NO: 20.

85. The method of any one of Items 39-42, wherein the second antigen binding domain is capable of binding CD20 or a variant thereof.

86. The method of any one of Items 39-42, and 85, wherein the second antigen binding domain is capable of binding CD20.

87. The method of any one of Items 39-42, 85 and 86, wherein the second antigen binding domain comprises an HCDR1 according to SEQ ID NO: 43, an HCDR2 according to SEQ ID NO: 44, and an HCDR3 according to SEQ ID NO: 107.

88. The method of any one of Items 39-42, and 85-87, wherein the second antigen binding domain comprises an LCDR1 according to SEQ ID NO: 39, an LCDR2 according to SEQ ID NO: 40, and an LCDR3 according to SEQ ID NO: 41.

89. The method of any one of Items 39-42, 85 and 86, wherein the first antigen binding domain comprises a heavy chain variable domain (VH) comprising an amino acid sequence according to SEQ ID NO: 43, an amino acid sequence according to SEQ ID NO: 44, and an amino acid sequence according to SEQ ID NO: 107 arranged non-contiguously from N-terminus to C-terminus.

90. The method of any one of Items 39-42, and 85-88, wherein the second antigen binding domain comprises a heavy chain variable domain (VH) comprising an amino acid sequence that is at least 80%, at least 85%, at least 90%, at least 95%, at least 96%, at least 97%, at least 98%, at least 99%, or 100% identical to an amino acid sequence according to SEQ ID NO: 42.

91. The method of any one of Items 39-42, 85, 86 and 89, wherein the first antigen binding domain comprises a light chain variable domain (VL) comprising an amino acid sequence according to SEQ ID NO: 39, an amino acid sequence according to SEQ ID NO: 40, and an amino acid sequence according to SEQ ID NO: 41 arranged non-contiguously from N-terminus to C-terminus.

92. The method of any one of Items 39-42, 85-88 and 90, wherein the second antigen binding domain comprises a light chain variable domain (VL) comprising an amino acid sequence that is at least 80% identical, at least 80%, at least 85%, at least 90%, at least 95%, at least 96%, at least 97%, at least 98%, at least 99%, or 100% identical to an amino acid sequence according to SEQ ID NO: 38.

93. The method of any one of Items 39-42, wherein the second antigen binding site is capable of binding CD22 or a variant thereof.

94. The method of any one of Items 39-42, and 93, wherein the second antigen is capable of binding CD22.

95. The method of any one of Items 39-42, 93 and 94, wherein the second antigen binding domain comprises an HCDR1 according to SEQ ID NO: 47, an HCDR2 according to SEQ ID NO: 48, and an HCDR3 according to SEQ ID NO: 49.

96. The method of any one of Items 39-42 and 93-95, wherein the second antigen binding domain comprises an LCDR1 according to SEQ ID NO: 51, an LCDR2 according to SEQ ID NO: 52, and an LCDR3 according to SEQ ID NO: 53.

97. The method of any one of Items 39-42, 93 and 94, wherein the first antigen binding domain comprises a heavy chain variable domain (VH) comprising an amino acid sequence according to SEQ ID NO: 47, an amino acid sequence according to SEQ ID NO: 48, and an amino acid sequence according to SEQ ID NO: 49 arranged non-contiguously from N-terminus to C-terminus.

98. The method of any one of Items 39-42, and 93-96, wherein the second antigen binding domain comprises a heavy chain variable domain (VH) comprising an amino acid sequence that is at least 80%, at least 85%, at least 90%, at least 95%, at least 96%, at least 97%, at least 98%, at least 99%, or 100% identical to an amino acid sequence according to SEQ ID NO: 46.

99. The method of any one of Items 39-42, 93, 94 and 97, wherein the first antigen binding domain comprises a light chain variable domain (VL) comprising an amino acid sequence according to SEQ ID NO: 51, an amino acid sequence according to SEQ ID NO: 52, and an amino acid sequence according to SEQ ID NO: 53 arranged non-contiguously from N-terminus to C-terminus.

100. The method of any one of Items 39-42, 93-96 and 98, wherein the second antigen binding domain comprises a light chain variable domain (VL) comprising an amino acid sequence that is at least 80%, at least 85%, at least 90%, at least 95%, at least 96%, at least 97%, at least 98%, at least 99%, or 100% identical to an amino acid sequence according to SEQ ID NO: 50.

101. The method of any one of Items 3-100, wherein the one or more targeted therapies comprise a fourth immunotherapeutic agent.

102. The method of any one of Items 3-101, wherein the one or more targeted therapies comprise a fourth population of engineered cells.

103. The method of Item 102, wherein the fourth population of engineered cells is directed to the second therapeutic target.

104. The method of Item 102 or 103, wherein the second population of engineered cells and the fourth population of engineered cells comprise engineered cells that are directed to the same therapeutic target.

105. The method of any one of Items 102-104, wherein the second population of engineered cells and the third population of engineered cells are directed to different therapeutic targets.

106. The method of any one of Items 102-105, wherein the fourth population of engineered cells comprises a fourth immunotherapeutic agent.

107. The method of Item 106 wherein the second immunotherapeutic agent and the fourth immunotherapeutic agent are the same.

108. The method of Item 106, wherein the second immunotherapeutic agent and the fourth immunotherapeutic agent are different.

109. The method of Item of any one of Items 106-108, wherein the fourth immunotherapeutic agent comprises a fourth antigen binding domain.

110. The method of Item 109, wherein the second antigen binding domain and the fourth antigen binding domain are the same.

111. The method of Item 109, wherein the second antigen binding domain and the fourth antigen binding domain are different.

112. The method of any one of Items 106-111, wherein the fourth immunotherapeutic agent comprises an antibody, a Fab, an scFV, an scFV-Fc, an scFV zipper, a diabody, a minibody, a CAR, a CAAR, a CAAR-T cell, a BAR, or a BAR-T cell.

113. The method of any one of Items 102-112, wherein the fourth population of engineered cells is a fourth population of engineered CAR-T cells.

114. The method of Item 113, wherein the second population of engineered CAR-T cells and the fourth population of engineered CAR-T cells comprise engineered CAR-T cells that are directed to the same therapeutic target.

115. The method of Item 113 or 114, wherein the second population of engineered CAR-T cells and the fourth population of engineered CAR-T cells comprise engineered CAR-T cells that are directed to different therapeutic targets.

116. The method of any one of Items 113-115, wherein the one or more targeted therapies comprises a fourth population of engineered CAR-T cells, wherein the fourth population of engineered CAR-T cells comprises one or more chimeric antigen receptors (CARs), and wherein at least one CAR comprises a fourth antigen binding domain.

117. The method of any one of Items 3-7, and 9-116, wherein the one or more targeted therapies comprise a failed therapy.

118. The method of Item 117, wherein the failed therapy is characterized by one or more of: (a) a plateau or increase in one or more symptom of the disease, (b) a plateau or a worsening of the extent or state of the disease, (c) a plateau or a worsening of disease progression, (d) an attenuated response to therapy, and (e) disease recurrence.

119. The method of any one of Items 109-118, wherein the fourth antigen binding domain comprises an HCDR1 according to SEQ ID NO: 26, an HCDR2 according to SEQ ID NO: 27, and an HCDR3 according to SEQ ID NO: 28.

120. The method of any one of Items 109-119, wherein the fourth antigen binding domain comprises an LCDR1 according to SEQ ID NO: 21, an LCDR2 according to SEQ ID NO: 22, and an LCDR3 according to SEQ ID NO: 23.

121. The method of any one of Items 109-120, wherein the fourth antigen binding domain comprises a heavy chain variable domain (VH) comprising an amino acid sequence that is at least 80%, at least 85%, at least 90%, at least 95%, at least 96%, at least 97%, at least 98%, at least 99%, or 100% identical to an amino acid sequence according to SEQ ID NO: 25.

122. The method of any one of Items 109-121, wherein the fourth antigen binding domain comprises a light chain variable domain (VL) comprising an amino acid sequence that is at least 80% identical, at least 80%, at least 85%, at least 90%, at least 95%, at least 96%, at least 97%, at least 98%, at least 99%, or 100% identical to an amino acid sequence according to SEQ ID NO: 20.

123. The method of any one of Items 109-118, wherein the fourth antigen binding domain comprises an HCDR1 according to SEQ ID NO: 43, an HCDR2 according to SEQ ID NO: 44, and an HCDR3 according to SEQ ID NO: 107.

124. The method of anyone of Items 109-118 and 123, wherein the fourth antigen binding domain comprises an LCDR1 according to SEQ ID NO: 39, an LCDR2 according to SEQ ID NO: 40, and an LCDR3 according to SEQ ID NO: 41.

125. The method of any one of Items 109-118, 123 and 124, wherein the fourth antigen binding domain comprises a heavy chain variable domain (VH) comprising an amino acid sequence that is at least 80% identical, at least 80%, at least 85%, at least 90%, at least 95%, at least 96%, at least 97%, at least 98%, at least 99%, or 100% identical to an amino acid sequence according to SEQ ID NO: 42.

126. The method of any one of Items 109-118 and 123-125, wherein the fourth antigen binding domain comprises a light chain variable domain (VL) comprising an amino acid sequence that is at least 80% identical, at least 80%, at least 85%, at least 90%, at least 95%, at least 96%, at least 97%, at least 98%, at least 99%, or 100% identical to an amino acid sequence according to SEQ ID NO: 38.

127. The method of any one of Items 109-118, wherein the fourth antigen binding domain comprises an HCDR1 according to SEQ ID NO: 47, an HCDR2 according to SEQ ID NO: 48, and an HCDR3 according to SEQ ID NO: 49.

128. The method of any one of Items 109-118 and 127, wherein the fourth antigen binding domain comprises an LCDR1 according to SEQ ID NO: 51, an LCDR2 according to SEQ ID NO: 52, and an LCDR3 according to SEQ ID NO: 53.

129. The method of any one of Items 109-118, 127 and 128, wherein the fourth antigen binding domain comprises a heavy chain variable domain (VH) comprising an amino acid sequence that is at least 80% identical, at least 80%, at least 85%, at least 90%, at least 95%, at least 96%, at least 97%, at least 98%, at least 99%, or 100% identical to an amino acid sequence according to SEQ ID NO: 46.

130. The method of any one of Items 109-118 and 127-130, wherein the fourth antigen binding domain comprises a light chain variable domain (VL) comprising an amino acid sequence that is at least 80% identical, at least 80%, at least 85%, at least 90%, at least 95%, at least 96%, at least 97%, at least 98%, at least 99%, or 100% identical to an amino acid sequence according to SEQ ID NO: 50.

131. The method of any one of Items 1-130, wherein the patient is at risk of antigen evasion.

132. The method of any one of Items 1-131, wherein the patient is suspected of having antigen evasion.

133. The method of any one of Items 1-132, wherein the patient is at risk of antigen drift.

134. The method of any one of Items 1-133, wherein the patient is suspected of having antigen drift.

135. The method of any one of Item 1-134, wherein the patient is at risk of or suffering from cancer.

136. The method of any one of Items 1-135, wherein the cancer is a B cell malignancy.

137. The method of any one of Items 1-136, wherein the disease or disorder is characterized by antigen evasion.

138. The method of any one of Items 1-137, wherein the disease or disorder is prone to antigen evasion.

139. The method of any one of Items 1-138, wherein the disease or disorder is characterized by antigenic drift.

140. The method of any one of Items 1-139, wherein the disease or disorder is prone to antigenic drift.

141. The method of any one of Items 1-140, wherein the disease or disorder is cancer.

142. The method of Item 141, wherein the cancer is or comprises lymphoma, leukemia, B-cell acute lymphoblastic leukemia (B-ALL), B-cell Non-Hodgkin lymphoma (B-NHL), or B-cell chronic lymphoblastic leukemia.

143. The method of Item 141 or 142, wherein the cancer is or comprises lymphoma.

144. The method of Item 143, wherein the lymphoma is a B cell lymphoma.

145. The method of Item 141 or 142, wherein the cancer is or comprises leukemia.

146. The method of Item 141 or 142, wherein the cancer is or comprises B-cell acute lymphoblastic leukemia (B-ALL).

147. The method of Item 141 or 142, wherein the cancer is or comprises B-cell Non-Hodgkin lymphoma (B-NHL).

148. The method of Item 141 or 142, wherein the cancer is or comprises B-cell chronic lymphoblastic leukemia.

149. The method of Item 141 or 142, wherein the cancer comprises a B cell malignancy.

150. The method of Item 46, 49-54, 93-149, wherein engineered CAR-T cells of the first, second, and/or third population comprise one or more CARs comprising a leader sequence, CD8α signal peptide, a linker, an m971 binder-based scFv, a CD8α hinge domain, a CD8 transmembrane domain, a CD28 transmembrane domain, a 4-1BB costimulatory domain, a CD28 signaling domain, a CD137 signaling domain, a CD8 signaling domain, a CD3ζ signaling domain, or a combination thereof.

151. The method of Item 46, 49-54, 93-150, wherein engineered CAR-T cells of the first, second, and/or third population comprise one or more CARs comprise a CD8α transmembrane domain or a CD28 transmembrane domain.

152. The method of any one of Items 46, 49-54, 93-151, wherein engineered CAR-T cells of the first, second, and/or third population comprise one or more CARs comprise a CD137 signaling domain and a CD3ζ signaling domain.

153. The method of any one of Items 46, 49-54, 93-152, wherein engineered CAR-T cells of the first, second, and/or third population comprise one or more CARs comprise a CD28 signaling domain and a CD3ζ signaling domain.

154. The method of any one of Items 46 and 150-153, wherein engineered CAR-T cells of the first, second, and/or third population comprise one or more CARs comprise a CD28 signaling domain, a CD137 signaling domain, and a CD3ζ signaling domain.

155. The method of any one of Items 150-154, wherein the CD8α signal peptide comprises an amino acid sequence according to SEQ ID NO: 6.

156. The method of any one of Items 150-155, wherein the linker is selected from the group consisting of IgG linkers, Whitlow linkers, (G4S)n linkers, wherein n is 1, 2, 3, 4, or more, and modifications thereof.

157. The method of any one of Items 150-156, wherein the linker is a (G4S)n linker, wherein n is 1 or 3.

158. The method of any one of Items 150-157, wherein the CD8α hinge domain comprises an amino acid sequence according to SEQ ID NO: 9.

159. The method of any one of Items 150-158, wherein the CD8 transmembrane domain comprises an amino acid sequence according to SEQ ID NO: 14 or 86.

160. The method of any one of Items 150-159, wherein the CD28 transmembrane domain comprises an amino acid sequence according to SEQ ID NO: 15, 87, or 114.

161. The method of any one of Items 150-160, wherein the 4-1BB costimulatory domain comprises an amino acid sequence according to SEQ ID NO: 16.

162. The method of any one of Items 150-161, wherein the CD28 signaling domain comprises an amino acid sequence according to SEQ ID NO: 17 or 88.

163. The method of any one of Items 150-162, wherein the CD137 signaling domain comprises an amino acid sequence according to SEQ ID NO: 90.

164. The method of any one of Items 150-163, wherein the CD8 signaling domain comprises an amino acid sequence according to SEQ ID NO: 89.

165. The method of any one of Items 150-164, wherein the CD3ζ signaling domain comprises an amino acid sequence according to SEQ ID NO: 18 or 115.

166. The method of any one of Items 46, 49-54, 93-165, wherein engineered CAR-T cells of the first, second, and/or third population comprise one or more CARs comprise an amino acid sequence that is at least 80%, at least 85%, at least 90%, at least 95%, at least 96%, at least 97%, at least 98%, at least 99%, or 100% identical to an amino acid sequence according to SEQ ID NO: 91, 92, or 93.

167. The method of any one of Items 46-166, wherein engineered CAR-T cells of the first, second, and/or third population are propagated from a primary T cell or a progeny thereof, or are derived from a T cell differentiated from an iPSC or a progeny thereof.

168. The method of any one of Items 46-166, wherein engineered CAR-T cells of the first, second, and/or third population are differentiated cells derived from an induced pluripotent stem cell or a progeny thereof.

169. The method of any one of Items 46-166, wherein engineered CAR-T cells of the first, second, and/or third population are progeny of primary immune cells.

170. The method of any one of Items 46-169, wherein engineered CAR-T cells of the first, second, and/or third population are a CAR+ T cell, a CD4+ CAR+ T cell, or a CD8+ CAR+ T cell.

171. The method of any of any of Items 46-170, wherein engineered CAR-T cells of the first, second, and/or third population are autologous CAR-T cells.

172. The method of any one of Items 46-170, wherein engineered CAR-T cells of the first, second, and/or third population are allogeneic CAR-T cells.

173. The method of any one of Items 46-172, wherein engineered CAR-T cells of the first, second, and/or third population are primary cells.

174. The method of Item 173, wherein the primary cells are derived from a single donor.

175. The method of Item 173 or 174, wherein the primary cells are derived from two or more donors.

176. The method of Item 46-175, wherein engineered CAR-T cells of the first, second, and/or third population are derived from induced pluripotent stem cells (iPSCs).

177. The method of Item 176, wherein the iPSCs are derived from a single donor.

178. The method of Item 176, wherein the iPSCs are derived from two or more donors.

179. The method of any one of Items 46-178, wherein engineered cells (e.g., engineered CAR-T cells) of the first, second, and/or third population comprise reduced expression of a functional major histocompatibility complex class I human leukocyte antigen (HLA-1) complex relative to an unaltered or unmodified wild-type or control cell.

180. The method of any one of Items 46-179, wherein engineered cells (e.g., engineered CAR-T cells) of the first, second, and/or third population comprise one or more genetic modifications that reduce expression of one or more HLA-I molecules or HLA I associated molecules relative to an unaltered or unmodified wild-type or control cell.

181. The method of any one of Items 46-180, wherein engineered cells (e.g., engineered CAR-T cells) of the first, second, and/or third population do not express one or more HLA-I molecules or HLA I associated molecules.

182. The method of any one of Items 179-181, wherein the one or more HLA-I molecules comprise HLA-A, HLA-B, HLA-C, or a combination thereof.

183. The method of any one of Items 180-182, wherein the one or more HLA-I molecules comprise HLA-A.

184. The method of any one of Items 180-183, wherein the one or more HLA-I molecules comprise HLA-B.

185. The method of any one of Items 180-184, wherein the one or more HLA-I molecules comprise HLA-C.

186. The method of any one of Items 180-185, wherein the one or more HLA-I associated molecules comprise β-2 microglobulin (B2M).

187. The method of any one of Items 46-186, wherein engineered cells (e.g., engineered CAR-T cells) of the first, second, and/or third population comprise reduced expression of a functional major histocompatibility complex class 11 human leukocyte antigen (HLA-II) complex relative to an unaltered or unmodified wild-type or control cell.

188. The method of any one of Items 46-187, wherein engineered cells (e.g., engineered CAR-T cells) of the first, second, and/or third population comprise one or more genetic modifications that reduce expression of one or more HLA-II molecules or HLA II associated molecules relative to an unaltered or unmodified wild-type or control cell.

189. The method of any one of Items 46-188, wherein engineered cells (e.g., engineered CAR-T cells) of the first, second, and/or third population do not express one or more HLA-II molecules or HLA II associated molecules.

190. The method of Item 188 or 189, wherein the one or more HLA-II molecules comprise HLA-DP, HLA-DM, HLA-DOB, HLA-DQ, HLA-DR, or a combination thereof.

191. The method of any one of Items 188-190, wherein the one or more HLA-II molecules comprise HLA-DP.

192. The method of any one of Items 188-191, wherein the one or more HLA-II molecules comprise HLA-DM.

193. The method of any one of Items 188-192, wherein the one or more HLA-II molecules comprise HLA-DOB.

194. The method of any one of Items 188-193, wherein the one or more HLA-II molecules comprise HLA-DQ.

195. The method of any one of Items 188-194, wherein the one or more HLA-II molecules comprise HLA-DR.

196. The method of any one of Items 188-195, wherein the one or more HLA-II associated molecules comprise MHC class II transactivator (CIITA).

197. The method of any one of Items 46-196, wherein engineered cells (e.g., engineered CAR-T cells) of the first, second, and/or third population comprise reduced expression of RHD, ABO, PCDH11Y, NLGN4Y, or a combination thereof relative to an unaltered or unmodified wild-type or control cell.

198. The method of any one of Items 46-197, wherein engineered cells (e.g., engineered CAR-T cells) of the first, second, and/or third population comprise one or more genetic modifications that reduce expression of RHD, ABO, PCDH11Y, NLGN4Y, or a combination thereof relative to an unaltered or unmodified wild-type or control cell.

199. The method of any one of Items 46-198, wherein engineered cells (e.g., engineered CAR-T cells) of the first, second, and/or third population do not express RHD, ABO, PCDH11Y, NLGN4Y, or a combination thereof.

200. The method of any one of Items 46-199, wherein engineered cells (e.g., engineered CAR-T cells) of the first, second, and/or third population comprise reduced expression of a T cell receptor (TCR) relative to an unaltered or unmodified wild-type or control cell.

201. The method of any one of Items 46-200, wherein engineered cells (e.g., engineered CAR-T cells) of the first, second, and/or third population comprise one or more genetic modifications that reduce expression of a T cell receptor (TCR) relative to an unaltered or unmodified wild-type or control cell.

202. The method of Item 201, wherein the TCR is a TCR-alpha (TRAC) and/or a TCR-beta (TRBC).

203. The method of any one of Items 46-202, wherein engineered cells (e.g., engineered CAR-T cells) of the first, second, and/or third population do not express TRAC and/or TRBC.

204. The method any one of Items 201-203, wherein the TCR is a TRAC.

205. The method of any one of Items 201-203, wherein the TCR is a TRBC.

206. The method of any one of Items 46-205, wherein engineered cells (e.g., engineered CAR-T cells) of the first, second, and/or third population comprise reduced expression of CD52 and/or CD70 relative to an unaltered or unmodified wild-type or control cell.

207. The method of any one of Items 46-206, wherein engineered cells (e.g., engineered CAR-T cells) of the first, second, and/or third population comprise one or more genetic modifications that reduce expression of CD52 and/or CD70 relative to an unaltered or unmodified wild-type or control cell.

208. The method of any one of Items 46-207, wherein engineered cells (e.g., engineered CAR-T cells) of the first, second, and/or third population do not express CD52 and/or CD70.

209. The method of any one of Items 46-208, wherein engineered cells (e.g., engineered CAR-T cells) of the first, second, and/or third population comprise reduced expression of PD-1 relative to an unaltered or unmodified wild-type or control cell.

210. The method of any one of Items 46-209, wherein engineered cells (e.g., engineered CAR-T cells) of the first, second, and/or third population comprise one or more genetic modifications that reduce expression of PD-1 relative to an unaltered or unmodified wild-type or control cell.

211. The method of any one of Items 46-210, wherein engineered cells (e.g., engineered CAR-T cells) of the first, second, and/or third population do not express PD-1.

212. The method of any one of Items 180-211, wherein the one or more genetic modifications comprise one or more gene knock downs.

213. The method of any one of Items 180-212, wherein the one or more genetic modifications are introduced by RNA silencing or RNA interference (RNAi).

214. The method of Item 213, wherein RNA silencing or RNA interference (RNAi) comprises contacting a parental cell of the first engineered cell with short interfering RNAs (siRNAs), PIWI-interacting RNAs (piRNAs), short hairpin RNAs (shRNAs), and microRNAs (miRNAs).

215. The method of any one of Items 180-214, wherein the one or more genetic modifications are introduced by inducing an insertion or a deletion in the gene using a gene editing system.

216. The method of any one of Items 46-215, wherein engineered cells (e.g., engineered CAR-T cells) of the first, second, and/or third population comprise a genome editing system.

217. The method of Item 216, wherein the gene editing system comprises a zinc finger nuclease (ZFN), a transcription activator-like effector nuclease (TALENs), a meganuclease, a transposase, a clustered regularly interspaced short palindromic repeat (CRISPR)/Cas system, a nickase system, a base editing system, a prime editing system, and/or a gene writing system.

218. The method of Item 216 or 217, wherein the genome editing system comprises a genome targeting entity and a genome modifying entity.

219. The method of Item 218, wherein the genome targeting entity comprises a nucleic acid-guided targeting entity.

220. The method of Item 218 or 219, wherein the genome targeting entity comprises a sequence specific nuclease, a nucleic acid programmable DNA binding protein, an RNA guided nuclease, RNA-guided nuclease comprising a Cas nuclease and a guide RNA (CRISPR-Cas combination), a ribonucleoprotein (RNP) complex comprising a gRNA and a Cas nuclease, a homing endonuclease, a zinc finger nuclease (ZF) nucleic acid binding entity, a transcription activator-like effector (TALE) nucleic acid binding entity, a meganuclease, a Cas nuclease, a core Cas protein, a homing endonuclease, an endonuclease-deficient-Cas protein, an enzymatically inactive Cas protein, a CRISPR-associated transposase (CAST), a Type II or Type V Cas protein, or a functional portion thereof.

221. The method of any one of Items 218-220, wherein the genome targeting entity comprises Cas1, Cas2, Cas3, Cas4, Cas5, Cas6, Cas7, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr1, Cmr2, Cmr3, Cmr4, Cmr5, Cmr6, Csd1, Csd2, Cas5d, Cse1, Cse2, Cse3, Cse4, Cas5e, Csf1, Csm1, Csm2, Csm3, Csm4, Csm5, Csn1, Csn2, Cst1, Cst2, Cas5t, Csh1, Csh2, Cas5h, Csa1, Csa2, Csa3, Csa4, Csa5, Cas5a, Csx10, Csx11, Csy1, Csy2, Csy3, Csy4, Mad7, SpCas9, eSpCas9, SpCas9-HF1, HypaSpCas9, HeFSpCas9, and evoSpCas9 high-fidelity variants of SpCas9, SaCas9, NmeCas9, CjCas9, StCas9, TdCas9, LbCas12a, AsCas12a, AacCas12b, BhCas12b v4, TnpB, dCas (D10A), dCas (H840A), dCas13a, dCas13b, or a functional portion thereof.

222. The method of any one of Items 218-221, wherein the genome modifying entity cleaves, deaminates, nicks, polymerizes, interrogates, integrates, cuts, unwinds, breaks, alters, methylates, demethylates, or otherwise destabilizes the target locus.

223. The method of any one of Items 218-222, wherein the genome modifying entity comprises a recombinase, integrase, transposase, endonuclease, exonuclease, nickase, helicase, DNA polymerase, RNA polymerase, reverse transcriptase, deaminase, flippase, methylase, demethylase, acetylase, a nucleic acid modifying protein, an RNA modifying protein, a DNA modifying protein, an Argonaute protein, an epigenetic modifying protein, a histone modifying protein, or a functional portion thereof.

224. The method of any one of Items 218-223, wherein the genome modifying entity comprises a sequence specific nuclease, a nucleic acid programmable DNA binding protein, an RNA guided nuclease, RNA-guided nuclease comprising a Cas nuclease and a guide RNA (CRISPR-Cas combination), a ribonucleoprotein (RNP) complex comprising the gRNA and the Cas nuclease, a homing endonuclease, a zinc finger nuclease (ZFN), a transcription activator-like effector nuclease (TALEN), a meganuclease, a Cas nuclease, a core Cas protein, a TnpB nuclease, an endonuclease-deficient-Cas protein, an enzymatically inactive Cas protein, a CRISPR-associated transposase (CAST), a Type II or Type V Cas protein, base editing, prime editing, a Programmable Addition via Site-specific Targeting Elements (PASTE), or a functional portion thereof.

225. The method of any one of Items 218-224, wherein the genome modifying entity comprises Cas1, Cas2, Cas3, Cas4, Cas5, Cas6, Cas7, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr1, Cmr2, Cmr3, Cmr4, Cmr5, Cmr6, Csd1, Csd2, Cas5d, Cse1, Cse2, Cse3, Cse4, Cas5e, Csf1, Csm1, Csm2, Csm3, Csm4, Csm5, Csn1, Csn2, Cst1, Cst2, Cas5t, Csh1, Csh2, Cas5h, Csa1, Csa2, Csa3, Csa4, Csa5, Cas5a, Csx10, Csx11, Csy1, Csy2, Csy3, Csy4, Mad7, SpCas9, eSpCas9, SpCas9-HF1, HypaSpCas9, HeFSpCas9, and evoSpCas9 high-fidelity variants of SpCas9, SaCas9, NmeCas9, CjCas9, StCas9, TdCas9, LbCas12a, AsCas12a, AacCas12b, BhCas12b v4, TnpB, Fokl, dCas (D10A), dCas (H840A), dCas13a, dCas13b, a base editor, a prime editor, a target-primed reverse transcription (TPRT) editor, APOBECI, cytidine deaminase, adenosine deaminase, uracil glycosylase inhibitor (UGI), adenine base editors (ABE), cytosine base editors (CBE), reverse transcriptase, serine integrase, recombinase, transposase, polymerase, adenine-to-thymine or “ATBE” (or thymine-to-adenine or “TABE”) transversion base editor, ten-eleven translocation methylcytosine dioxygenases (TETs), TET1, TET3, TET1CD, histone acetyltransferase p300, histone methyltransferase SMYD3, histone methyltransferase PRDM9, H3K79 methyltransferase DOT1L, transcriptional repressor, or a functional portion thereof.

226. The method of any one of Items 218-225, wherein the genome targeting entity and the genome modifying entity are different domains of a single polypeptide.

227. The method of any one of Items 218-226, wherein the genome targeting entity and genome modifying entity are two different polypeptides that are operably linked together.

228. The method of any one of Items 218-226, wherein the genome targeting entity and genome modifying entity are two different polypeptides that are not linked together.

229. The method of any one of Items 218-228, wherein the genome modifying entity comprises a guide nucleic acid having a targeting domain that is complementary to at least one sequence within the genomic safe harbor site, optionally wherein the guide nucleic acid is a guide RNA (gRNA).

230. The method of any one of Items 218-229, wherein the genome modifying entity is an RNA-guided nuclease.

231. The method of Item 230, wherein the RNA-guided nuclease comprises a Cas nuclease and a guide RNA (CRISPR-Cas combination).

232. The method of Item 231, wherein the CRISPR-Cas combination is a ribonucleoprotein (RNP) complex comprising the gRNA and the Cas nuclease.

233. The method of Item 232, wherein the Cas nuclease is a Type II or Type V Cas protein.

234. The method of Item 232 or 233, wherein the Cas nuclease is Cas3, Cas4, Cas5, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr5, Cse1, Cse2, Csf1, Csm2, Csn2, Csx10, Csx11, Csy1, Csy2, Csy3, or Mad7.

235. The method of any one of Items 180-234, wherein the one or more genetic modifications are made at a modification site.

236. The method of Item 235, wherein the modification site is 25 nucleotides or less from a protospacer adjacent motif (PAM) sequence, wherein the PAM sequence is ngg, nag, ngrrt, ngrrn, nnnngatt, nnnnryac, nnagaaw, naaaac, tttv, ttn, attn, tttn, gttn, or yttn and wherein:
(i) r=a or g,
(ii) y=c or t,
(iii) w=a or t,
(iv) v=a or c or g, and
(v) n=a, c, t, or g.

237. The method of any one of Items 180-236, wherein the one or more genetic modifications are introduced using homology-directed repair (HDR)-mediated modification using SpCas9 and the PAM is ngg or nag, wherein n=a, c, t, or g.

238. The method of any one of Items 180-236, wherein the one or more genetic modifications are introduced using homology-directed repair (HDR)-mediated modification using SaCas9 and the PAM is ngrrt or ngrrn, wherein:
(vi) r=a or g, and
(vii) n=a, c, t, or g.

239. The method of any one of Items 180-236, wherein the one or more genetic modifications are introduced using homology-directed repair (HDR)-mediated modification using NmeCas9 and the PAM is nnnngatt, wherein n=a, c, t, or g.

240. The method of any one of Items 180-236, wherein the one or more genetic modifications are introduced using homology-directed repair (HDR)-mediated modification using CjCas9 and the PAM is nnnnryac, wherein:
(viii) r=a or g,
(ix) y=c or t, and
(x) n=a, c, t, or g.

241. The method of any one of Items 180-236, wherein the one or more genetic modifications are introduced using homology-directed repair (HDR)-mediated modification using StCas9 and the PAM is nnagaaw wherein:
(xi) w=a or t, and
(xii) n=a, c, t, or g.

242. The method of any one of Items 180-236, wherein the one or more genetic modifications are introduced using homology-directed repair (HDR)-mediated modification using TdCas9 and the PAM is naaaac, wherein n=a, c, t, or g.

243. The method of any one of Items 180-236, wherein the one or more genetic modifications are introduced using homology-directed repair (HDR)-mediated modification using LbCas12a and the PAM is tttv, wherein v=a or c or g.

244. The method of any one of Items 180-236, wherein the one or more genetic modifications are introduced using homology-directed repair (HDR)-mediated modification using AsCas12a and the PAM is tttv, wherein v=a or c or g.

245. The method of any one of Items 180-236, wherein the one or more genetic modifications are introduced using homology-directed repair (HDR)-mediated modification using AacCas12b and the PAM is ttn, wherein n=a, c, t, or g.

246. The method of any one of Items 180-236, wherein the one or more genetic modifications are introduced using homology-directed repair (HDR)-mediated modification using BhCas12b and the PAM is attn., tttn, or gttn, wherein n=a, c, t, or g.

247. The method of any one of Items 180-236, wherein the one or more genetic modifications are introduced using homology-directed repair (HDR)-mediated modification using MAD7 (ErCas12a) and the PAM is yttn, wherein:
(xiii) y=c or t, and
(xiv) n=a, c, t, or g.

248. The method of any one of Items 180-247, the one or more genetic modifications are introduced by inducing an insertion or a deletion in the gene using a gene editing system ex vivo from a donor subject.

249. The method of any one of Items 46-248, wherein engineered cells (e.g., engineered CAR-T cells) of the first, second, and/or third population comprise one or more exogenous polynucleotides that encode one or more tolerogenic factors.

250. The method of Item 249, wherein the one or more tolerogenic factors comprise A20/TNFAIP3, C1-Inhibitor, CCL21, CCL22, CD16, CD16 Fc receptor, CD24, CD27, CD35, CD39, CD46, CD47, CD52, CD55, CD59, CD64, CD200, CR1, CTLA4-Ig, DUX4, FasL, H2-M3, HLA-C, HLA-E, HLA-E heavy chain, HLA-F, HLA-G, IDO1, IL-10, IL15-RF, IL-35, IL-39, MANF, Mfge8, PD-L1, Serpinb9, CCL21, CCL22, B2M-HLA-E, C1 inhibitor, CR1, or a combination thereof.

251. The method of any one of Items 46-250, wherein engineered cells (e.g., engineered CAR-T cells) of the first, second, and/or third population comprise an exogenous polynucleotides that encode CD24.

252. The method of any one of Items 46-251, wherein engineered cells (e.g., engineered CAR-T cells) of the first, second, and/or third population comprise an exogenous polynucleotides that encode CD47.

253. The method of any one of Items 46-252, wherein engineered cells (e.g., engineered CAR-T cells) of the first, second, and/or third population comprise an exogenous polynucleotides that encode CD52.

254. The method of any one of Items 46-253, wherein engineered cells (e.g., engineered CAR-T cells) of the first, second, and/or third population comprise an exogenous polynucleotides that encode CD70.

255. The method of any one of Items 46-254, wherein engineered cells (e.g., engineered CAR-T cells) of the first, second, and/or third population express A20/TNFAIP3, C1-Inhibitor, CCL21, CCL22, CD16, CD16 Fc receptor, CD24, CD27, CD35, CD39, CD46, CD47, CD52, CD55, CD59, CD64, CD200, CR1, CTLA4-Ig, DUX4, FasL, H2-M3, HLA-C, HLA-E, HLA-E heavy chain, HLA-F, HLA-G, IDO1, IL-10, IL15-RF, IL-35, IL-39, MANF, Mfge8, PD-L1, Serpinb9, CCL21, CCL22, B2M-HLA-E, C1 inhibitor, CR1, and any combination thereof from one or more exogenous polynucleotides.

256. The method of any one of Items 46-255, wherein engineered cells (e.g., engineered CAR-T cells) of the first, second, and/or third population express CD47, HLA-E, and PD-L1 from one or more exogenous polynucleotides.

257. The method of any one of Items 46-256, wherein engineered cells (e.g., engineered CAR-T cells) of the first, second, and/or third population express CD24 from an exogenous polynucleotide.

258. The method of any one of Items 46-257, wherein engineered cells (e.g., engineered CAR-T cells) of the first, second, and/or third population express CD47 from an exogenous polynucleotide.

259. The method of any one of Items 46-258, wherein engineered cells (e.g., engineered CAR-T cells) of the first, second, and/or third population express CD52 from an exogenous polynucleotide.

260. The method of any one of Items 46-259, wherein engineered cells (e.g., engineered CAR-T cells) of the first, second, and/or third population express CD70 from an exogenous polynucleotide.

261. The method of any one of Items 46-260, wherein engineered cells (e.g., engineered CAR-T cells) of the first, second, and/or third population express CD47 from one or more exogenous polynucleotides.

262. The method of Item 261, wherein one or more exogenous polynucleotides encoding one or more tolerogenic factors and/or one or more exogenous polynucleotides encoding one or more CARs are introduced at a safe harbor locus, a target locus, an RHD locus, a B2M locus, a CIITA locus, a TRAC locus, or a TRB locus.

263. The method of Item 262, wherein the safe harbor locus is a CCR5 locus, a PPP1R12C locus, a CLYBL locus, or a Rosa locus.

264. The method of Item 262, wherein the target locus is a CXCR4 locus, an ALB locus, a SHS231 locus, an F3 (CD142) locus, a MICA locus, a MICB locus, a LRP1 (CD91) locus, a HMGB1 locus, an ABO locus, a FUT1 locus, or a KDM5D locus.

265. The method of any one of Items 249-264, wherein one or more exogenous polynucleotides encoding one or more tolerogenic factors and/or one or more exogenous polynucleotides encoding one or more CARs are introduced into the first engineered cell using a gene therapy vector or a transposase system.

266. The method of Item 265, wherein the transposase system comprises a transposase, a PiggyBac transposon, a Sleeping Beauty (SB11) transposon, a Mos1 transposon, or a Tol2 transposon.

267. The method of Item 265, wherein the gene therapy vector is a retrovirus or a fusosome.

268. The method of any one of Items 249-267, wherein one or more exogenous polynucleotides encoding one or more tolerogenic factors and/or one or more exogenous polynucleotides encoding one or more CARs are encoded by a polycistronic vector.

269. The method of Item 268, wherein the polycistronic vector is a bicistronic vector comprising one exogenous polynucleotide encoding a tolerogenic factor and one exogenous polynucleotide encoding one or more CARs.

270. The method of any one of Items 46-269, wherein engineered cells (e.g., engineered CAR-T cells) of the first, second, and/or third population comprise reduced expression of a HLA-I complex or reduced expression of a HLA-II complex relative to an unaltered or unmodified wild-type or control cell.

271. The method of any one of Items 46-270, wherein engineered cells (e.g., engineered CAR-T cells) of the first, second, and/or third population comprise reduced expression of B2M or reduced expression of CIITA relative to an unaltered or unmodified wild-type or control cell.

272. The method of any one of Items 46-271, wherein engineered cells (e.g., engineered CAR-T cells) of the first, second, and/or third population comprise (i) reduced expression of a HLA-I complex and (ii) reduced expression of a HLA-II complex relative to an unaltered or unmodified wild-type or control cell.

273. The method of any one of Items 46-272, wherein engineered cells (e.g., engineered CAR-T cells) of the first, second, and/or third population comprise (i) reduced expression of B2M and (ii) reduced expression of CIITA relative to an unaltered or unmodified wild-type or control cell.

274. The method of any one of Items 46-273, wherein engineered cells (e.g., engineered CAR-T cells) of the first, second, and/or third population comprise (i) reduced expression of a HLA-I complex or reduced expression of a HLA-II complex relative to an unaltered or unmodified wild-type or control cell, and (ii) increased expression of CD47 relative to an unaltered or unmodified wild-type or control cell.

275. The method of any one of Items 46-274, wherein engineered cells (e.g., engineered CAR-T cells) of the first, second, and/or third population comprise (i) reduced expression of B2M or reduced expression of CIITA relative to an unaltered or unmodified wild-type or control cell, and (ii) increased expression of CD47 relative to an unaltered or unmodified wild-type or control cell.

276. The method of any one of Items 46-275, wherein engineered cells (e.g., engineered CAR-T cells) of the first, second, and/or third population comprise (i) reduced expression of a HLA-I complex relative to an unaltered or unmodified wild-type or control cell, (ii) reduced expression of a HLA-II complex relative to an unaltered or unmodified wild-type or control cell, and (iii) increased expression of CD47 relative to an unaltered or unmodified wild-type or control cell.

277. The method of any one of Items 46-277, wherein engineered cells (e.g., engineered CAR-T cells) of the first, second, and/or third population comprise (i) reduced expression of B2M relative to an unaltered or unmodified wild-type or control cell, (ii) reduced expression of CIITA relative to an unaltered or unmodified wild-type or control cell, and (iii) increased expression of CD47 relative to an unaltered or unmodified wild-type or control cell.

278. The method of any one of Items 46-277, wherein engineered cells (e.g., engineered CAR-T cells) of the first, second, and/or third population comprise (i) reduced expression of a HLA-I complex or reduced expression of a HLA-II complex, and (ii) reduced expression of a TCR relative to an unaltered or unmodified wild-type or control cell.

279. The method of any one of Item 46-278, wherein engineered cells (e.g., engineered CAR-T cells) of the first, second, and/or third population comprise (i) reduced expression of B2M or reduced expression of CIITA, and (ii) reduced expression of a TRAC relative to an unaltered or unmodified wild-type or control cell.

280. The method of any one of Items 46-279, wherein engineered cells (e.g., engineered CAR-T cells) of the first, second, and/or third population comprise (i) reduced expression of a HLA-I complex, (ii) reduced expression of a HLA-II complex, and (iii) reduced expression of a TCR relative to an unaltered or unmodified wild-type or control cell.

281. The method of any one of Items 46-280, wherein engineered cells (e.g., engineered CAR-T cells) of the first, second, and/or third population comprise (i) reduced expression of B2M, (ii) reduced expression of CIITA, and (iii) reduced expression of a TRAC relative to an unaltered or unmodified wild-type or control cell.

282. The method of any one of Items 46-280, wherein engineered cells (e.g., engineered CAR-T cells) of the first, second, and/or third population comprise (i) reduced expression of a HLA-I complex or reduced expression of a HLA-II complex relative to an unaltered or unmodified wild-type or control cell, (ii) reduced expression of a TRAC relative to an unaltered or unmodified wild-type or control cell, and (iii) increased expression of CD47 relative to an unaltered or unmodified wild-type or control cell.

283. The method of any one of Items 46-282, wherein engineered cells (e.g., engineered CAR-T cells) of the first, second, and/or third population comprise (i) reduced expression of B2M or reduced expression of CIITA relative to an unaltered or unmodified wild-type or control cell, (ii) reduced expression of a TRAC relative to an unaltered or unmodified wild-type or control cell, and (ii) increased expression of CD47 relative to an unaltered or unmodified wild-type or control cell.

284. The method of any one of Items 46-283, wherein engineered cells (e.g., engineered CAR-T cells) of the first, second, and/or third population comprise (i) reduced expression of a HLA-I complex relative to an unaltered or unmodified wild-type or control cell, (ii) reduced expression of a HLA-II complex relative to an unaltered or unmodified wild-type or control cell, (iii) reduced expression of a TRAC relative to an unaltered or unmodified wild-type or control cell, and (iv) increased expression of CD47 relative to an unaltered or unmodified wild-type or control cell.

285. The method of any one of Items 46-284, wherein engineered cells (e.g., engineered CAR-T cells) of the first, second, and/or third population comprise (i) reduced expression of B2M relative to an unaltered or unmodified wild-type or control cell, (ii) reduced expression of CIITA relative to an unaltered or unmodified wild-type or control cell, (iii) reduced expression of a TRAC relative to an unaltered or unmodified wild-type or control cell, and (iv) increased expression of CD47 relative to an unaltered or unmodified wild-type or control cell.

286. The method of any one of Items 46-285, wherein engineered cells (e.g., engineered CAR-T cells) of the first, second, and/or third population comprise (i) reduced expression of B2M or reduced expression of CD52, and (ii) reduced expression of a TRAC relative to an unaltered or unmodified wild-type or control cell.

287. The method of any one of Items 46-286, wherein engineered cells (e.g., engineered CAR-T cells) of the first, second, and/or third population comprise (i) reduced expression of B2M, (ii) reduced expression of CD52, and (iii) reduced expression of a TRAC relative to an unaltered or unmodified wild-type or control cell.

288. The method of any one of Items 46-287, wherein engineered cells (e.g., engineered CAR-T cells) of the first, second, and/or third population comprise (i) reduced expression of B2M or reduced expression of CD70, and (ii) reduced expression of a TRAC relative to an unaltered or unmodified wild-type or control cell.

289. The method of any one of Items 46-288, wherein engineered cells (e.g., engineered CAR-T cells) of the first, second, and/or third population comprise (i) reduced expression of B2M, (ii) reduced expression of CD70, and (iii) reduced expression of a TRAC relative to an unaltered or unmodified wild-type or control cell.

290. The method of any one of Items 46-289, wherein engineered cells (e.g., engineered CAR-T cells) of the first, second, and/or third population comprise (i) reduced expression of PD-1, and (ii) reduced expression of a TRAC relative to an unaltered or unmodified wild-type or control cell.

291. The method of any one of Items 246-290, wherein engineered cells (e.g., engineered CAR-T cells) of the first, second, and/or third population comprise B2M^{indel/indel}, CIITA^{indel/indel}, TRAC^{indel/indel}, and/or TRAC^{indel/indel} cells.

292. The method of any one of Items 1-291, wherein the disease or disorder is characterized by antigen evasion or antigenic drift, and wherein the one or more targeted therapies were administered to the patient prior to antigen evasion or antigenic drift.

293. The method of any one of Items 1-292, wherein the disease or disorder is characterized by antigen evasion or antigenic drift, and wherein the therapeutic agent is administered to the patient after antigen evasion or antigenic drift.

294. The method of any one of Items 1-293, wherein the patient is at risk of antigen evasion, and wherein the therapeutic agent is administered to the patient before antigen evasion.

295. The method of any one of Items 1-294, wherein the patient is at risk of antigen evasion, and wherein the therapeutic agent is administered to the patient before antigenic drift.

296. The method of any one of Items 1-295, wherein the patient has been diagnosed with the disease or disorder.

297. The method of any one of Items 1-296, further comprising evaluating the patient for the disease or disorder.

298. The method of any one of Items 1-297, wherein the patient comprises one or more cells that have undergone antigen evasion or antigenic drift.

299. The method of any one of Items 1-298, wherein the patient was evaluated for the presence of one or more cells that have undergone antigen evasion or antigenic drift.

300. The method of any one of Items 1-299, wherein the patient was evaluated before the population of engineered CAR-T cells was administered to the patient.

301. The method of any one of Items 1-300, further comprising evaluating the patient to determine if the patient comprises cells that have undergone antigen evasion or antigenic drift.

302. The method of any one of Items 1-301, wherein the patient is treated with an immunodepleting therapy prior to administering the therapeutic agent.

303. The method of Item 302, wherein the immunodepleting therapy administered prior to administering the therapeutic agent is at a lower dosage than the immunodepleting therapy administered to the patient prior to the one or more targeted therapies.

304. The method of Item 302 or 303, wherein the immunodepleting therapy comprises fewer doses than the immunodepleting therapy administered to the patient prior to the one or more targeted therapies.

305. The method of any one of Items 302-304, wherein the immunodepleting therapy comprises a reduced amount of immunodepleting agent than the immunodepleting therapy administered to the patient prior to the one or more targeted therapies.

306. The method of any one of Items 302-305, wherein the immunodepleting therapy comprises administration of fludarabine and/or cyclophosphamide.

307. The method of any one of Items 302-306, wherein the immunodepleting therapy comprises IV infusion of about 1-50 mg/m2 of fludarabine for about 1-7 days.

308. The method of any one of Items 302-307, wherein the immunodepleting therapy comprises IV infusion of about 1, about 5, about 10, about 20, about 30, about 40, or about 50 mg/m2 of fludarabine for about 1, about 2, about 3, about 4, about 5, about 6, or about 7 days.

309. The method of any one of Items 302-308, wherein the immunodepleting therapy comprises IV infusion of about 30 mg/m2 of fludarabine for about 5 days.

310. The method of any one of Items 302-309, wherein the immunodepleting therapy comprises IV infusion of about 30 mg/m2 of fludarabine for about 3 days.

311. The method of any one of Items 302-310, wherein the immunodepleting therapy comprises IV infusion of about 100-1000 mg/m2 of cyclophosphamide for about 1-7 days.

312. The method of any one of Items 302-311, wherein the immunodepleting therapy comprises IV infusion of about 100, about 200, about 300, about 400, about 500, about 600, about 700, about 800, about 900, or about 1000 mg/m2 of cyclophosphamide for about 1, about 2, about 3, about 4, about 5, about 6, or about 7 days.

313. The method of any one of Items 302-312, wherein the immunodepleting therapy comprises IV infusion of about 500 mg/m2 or more of cyclophosphamide for about 5 days.

314. The method of any one of Items 302-313, wherein the immunodepleting therapy further comprises IV infusion of about 3 mg, about 10 mg, or about 30 mg of alemtuzumab for about 1, about 2, about 3, about 4, about 5, about 6, or about 7 days.

315. The method of any one of Items 302-314, wherein the immunodepleting therapy comprises IV infusion of about 500 mg/m2 of cyclophosphamide for about 3 days.

316. The method of any one of Items 302-315, further comprising administering a second, third, fourth, fifth, or sixth dose of the therapeutic agent to the patient.

317. The method of Item 316, the patient is not treated with an immunodepleting therapy prior to the second, third, fourth, fifth, and/or sixth administration of the therapeutic agent.

318. The method of Item 316, wherein the patient is treated with an immunodepleting therapy prior to the second, third, fourth, fifth, and/or sixth administration of the therapeutic agent.

319. The method of Item 318, wherein the immunodepleting therapy that is administered prior to the second, third, fourth, fifth, and/or sixth administration of the therapeutic agent is (i) administration of fludarabine and/or cyclophosphamide, wherein the administration of fludarabine comprises IV infusion of about 1-50 mg/m2 of fludarabine for about 1-7 days, or (ii) the administration of cyclophosphamide comprises IV infusion of about 100-1000 mg/m2 of cyclophosphamide for about 1-7 days.

320. The method of any one of Items 1-319, wherein the therapeutic agent comprises a first population of engineered cells, and at least about 40×104 of engineered cells of the first population are administered to the patient.

321. The method of any one of Items 1-319, wherein the therapeutic agent comprises a first population of engineered cells, and up to about 8.0×108 engineered cells of the first population are administered to the patient, optionally wherein up to about 6.0×108 engineered cells of the first population are administered to the patient, optionally wherein about 1.0×106 to about 2.5×108 engineered cells of the first population are administered to the patient or wherein about 2.0×106 to about 2.0×108 engineered cells of the first population are administered to the patient.

322. The method of any one of Items 1-319, wherein the therapeutic agent comprises a first population of engineered cells, and up to about 6.0×108 engineered cells of the first population are administered to the patient in about 1-3 doses, optionally wherein (a) about 0.6×106 to about 6.0×108 engineered cells of the first population are administered to the patient in about 1-3 doses, (b) about 0.2×106 to about 5.0×106 engineered cells of the first population per kg of the patient's body weight are administered to the patient in about 1-3 doses, if the patient has a body weight of 50 kg or less, (c) about 0.1×108 to about 2.5×108 engineered cells of the first population are administered to the patient in about 1-3 doses, if the patient has a body weight greater than 50 kg, or (d) about 2.0×106 engineered cells of the first population per kg of the patient's body weight and up to about 2.0×108 engineered cells of the first population are administered to the patient in about 1-3 doses.

323. The method of any one of Items 1-319, wherein the therapeutic agent comprises a first population of engineered cells, and about 40×106 to about 200×106 engineered cells of the first population are administered to the patient, optionally wherein (a) about 40×106 to about 60×106 engineered cells of the first population are administered to the patient, (b) about 60×106 to about 80×106 engineered cells of the first population are administered to the patient, (c) about 80×106 to about 100×106 engineered cells of the first population are administered to the patient, (d) about 100×106 to about 120×106 engineered cells of the first population are administered to the patient, (e) about 120×106 to about 140×106 engineered cells of the first population are administered to the patient, (f) about 140×106 to about 160×106 engineered cells of the first population are administered to the patient, (g) about 160×106 to about 180×106 engineered cells of the first population are administered to the patient, or (h) about 180×106 to about 200×106 engineered cells of the first population are administered to the patient.

324. The method of any one of Items 1-319, wherein the therapeutic agent comprises a first population of engineered cells, and about 60×106 to about 120×106 engineered cells of the first population are administered to the patient, optionally wherein (a) about 60×106 to about 80×106 engineered cells of the first population are administered to the patient, (b) about 80×106 to about 100×106 engineered cells of the first population are administered to the patient, or (c) about 100×106 to about 120×106 engineered cells of the first population are administered to the patient.

325. The method of any one of Items 1-319, wherein the therapeutic agent comprises a first population of engineered cells, and about 120×106 to about 200×106 engineered cells of the first population are administered to the patient, (a) about 120×106 to about 140×106 engineered cells of the first population are administered to the patient, (b) about 140×106 to about 160×106 engineered cells of the first population are administered to the patient, (c) about 160×106 to about 180×106 engineered cells of the first population are administered to the patient, or (d) about 180×106 to about 200×106 engineered cells of the first population are administered to the patient.

326. The method of any one of Items 1-325, wherein the therapeutic agent comprises a second population of engineered cells, and at least about 40×104 of engineered cells of the second population are administered to the patient.

327. The method of any one of Items 1-325, wherein the therapeutic agent comprises a second population of engineered cells, and up to about 8.0×108 engineered cells of the second population are administered to the patient, optionally wherein up to about 6.0×108 engineered cells of the second population are administered to the patient, optionally wherein about 1.0×106 to about 2.5×108 engineered cells of the second population are administered to the patient or wherein about 2.0×106 to about 2.0×108 engineered cells of the second population are administered to the patient.

328. The method of any one of Items 1-325, wherein the therapeutic agent comprises a second population of engineered cells, and up to about 6.0×108 engineered cells of the second population are administered to the patient in about 1-3 doses, optionally wherein (a) about 0.6×106 to about 6.0×108 engineered cells of the second population are administered to the patient in about 1-3 doses, (b) about 0.2×106 to about 5.0×106 engineered cells of the second population per kg of the patient's body weight are administered to the patient in about 1-3 doses, if the patient has a body weight of 50 kg or less, (c) about 0.1×108 to about 2.5×108 engineered cells of the second population are administered to the patient in about 1-3 doses, if the patient has a body weight greater than 50 kg, or (d) about 2.0×106 engineered cells of the second population per kg of the patient's body weight and up to about 2.0×108 engineered cells of the second population are administered to the patient in about 1-3 doses.

329. The method any one of Items 1-325, wherein the therapeutic agent comprises a second population of engineered cells, and about 40×106 to about 200×106 engineered cells of the second population are administered to the patient, optionally wherein (a) about 40×106 to about 60×106 engineered cells of the second population are administered to the patient, (b) about 60×106 to about 80×106 engineered cells of the second population are administered to the patient, (c) about 80×106 to about 100×106 engineered cells of the second population are administered to the patient, (d) about 100×106 to about 120×106 engineered cells of the second population are administered to the patient, (e) about 120×106 to about 140×106 engineered cells of the second population are administered to the patient, (f) about 140×106 to about 160×106 engineered cells of the second population are administered to the patient, (g) about 160×106 to about 180×106 engineered cells of the second population are administered to the patient, or (h) about 180×106 to about 200×106 engineered cells of the second population are administered to the patient.

330. The method any one of Items 1-325, wherein the therapeutic agent comprises a second population of engineered cells, and about 60×106 to about 120×106 engineered cells of the second population are administered to the patient, optionally wherein (a) about 60×106 to about 80×106 engineered cells of the second population are administered to the patient, (b) about 80×106 to about 100×106 engineered cells of the second population are administered to the patient, or (c) about 100×106 to about 120×106 engineered cells of the second population are administered to the patient.

331. The method any one of Items 1-325, wherein the therapeutic agent comprises a second population of engineered cells, and about 120×106 to about 200×106 engineered cells of the second population are administered to the patient, (a) about 120×106 to about 140×106 engineered cells of the second population are administered to the patient, (b) about 140×106 to about 160×106 engineered cells of the second population are administered to the patient, (c) about 160×106 to about 180×106 engineered cells of the second population are administered to the patient, or (d) about 180×106 to about 200×106 engineered cells of the second population are administered to the patient.

332. The method of any one of Items 3-7, and 13-331, wherein the one or more targeted therapies comprise an autologous or allogeneic cell-based therapy, and wherein fewer or a lower number of engineered CAR-T cells are administered to the patient than were included in the prior therapy.

333. The method of any one of Items 1-325, wherein the therapeutic agent comprises a second population of engineered cells, and at least about 40×104 of engineered cells of the second population are administered to the patient.

334. The method of any one of Items 1-333, wherein the therapeutic agent comprises a third population of engineered cells, and up to about 8.0×108 engineered cells of the third population are administered to the patient, optionally wherein up to about 6.0×108 engineered cells of the third population are administered to the patient, optionally wherein about 1.0×106 to about 2.5×108 engineered cells of the third population are administered to the patient or wherein about 2.0×106 to about 2.0×108 engineered cells of the third population are administered to the patient.

335. The method of any one of Items 1-333, wherein the therapeutic agent comprises a third population of engineered cells, and up to about 6.0×108 engineered cells of the third population are administered to the patient in about 1-3 doses, optionally wherein (a) about 0.6×106 to about 6.0×108 engineered cells of the third population are administered to the patient in about 1-3 doses, (b) about 0.2×106 to about 5.0×106 engineered cells of the third population per kg of the patient's body weight are administered to the patient in about 1-3 doses, if the patient has a body weight of 50 kg or less, (c) about 0.1×108 to about 2.5×108 engineered cells of the third population are administered to the patient in about 1-3 doses, if the patient has a body weight greater than 50 kg, or (d) about 2.0×106 engineered cells of the third population per kg of the patient's body weight and up to about 2.0×108 engineered cells of the third population are administered to the patient in about 1-3 doses.

336. The method of any one of Items 1-333, wherein the therapeutic agent comprises a third population of engineered cells, and about 40×106 to about 200×106 engineered cells of the third population are administered to the patient, optionally wherein (a) about 40×106 to about 60×106 engineered cells of the third population are administered to the patient, (b) about 60×106 to about 80×106 engineered cells of the third population are administered to the patient, (c) about 80×106 to about 100×106 engineered cells of the third population are administered to the patient, (d) about 100×106 to about 120×106 engineered cells of the third population are administered to the patient, (e) about 120×106 to about 140×106 engineered cells of the third population are administered to the patient, (f) about 140×106 to about 160×106 engineered cells of the third population are administered to the patient, (g) about 160×106 to about 180×106 engineered cells of the third population are administered to the patient, or (h) about 180×106 to about 200×106 engineered cells of the third population are administered to the patient.

337. The method of any one of Items 1-333, wherein the therapeutic agent comprises a third population of engineered cells, and about 60×106 to about 120×106 engineered cells of the third population are administered to the patient, optionally wherein (a) about 60×106 to about 80×106 engineered cells of the third population are administered to the patient, (b) about 80×106 to about 100×106 engineered cells of the third population are administered to the patient, or (c) about 100×106 to about 120×106 engineered cells of the third population are administered to the patient.

338. The method of any one of Items 1-333, wherein the therapeutic agent comprises a third population of engineered cells, and about 120×106 to about 200×106 engineered cells of the third population are administered to the patient, (a) about 120×106 to about 140×106 engineered cells of the third population are administered to the patient, (b) about 140×106 to about 160×106 engineered cells of the third population are administered to the patient, (c) about 160×106 to about 180×106 engineered cells of the third population are administered to the patient, or (d) about 180×106 to about 200×106 engineered cells of the third population are administered to the patient.

339. The method of any one of Items 3-7, and 13-339, wherein the one or more targeted therapies comprise an autologous or allogeneic cell-based therapy, and wherein fewer or a lower number of engineered CAR-T cells are administered to the patient than were included in the prior therapy.

340. The method of any one of Items 1-339, wherein the therapeutic agent comprises a first population of engineered cells, and wherein the first population of engineered cells evade NK cell mediated cytotoxicity upon administration to the recipient patient.

341. The method of any one of Items 1-339, wherein the therapeutic agent comprises a first population of engineered cells, and wherein the first population of engineered cells are protected from cell lysis by mature NK cells upon administration to the recipient patient.

342. The method of any one of Items 1-339, wherein the therapeutic agent comprises a first population of engineered cells, and wherein the first population of engineered cells evade macrophage-mediated cytotoxicity, optionally wherein the macrophage-mediated cytotoxicity involves phagocytosis and/or reactive oxygen species.

343. The method of any one of Items 1-339, wherein the therapeutic agent comprises a first population of engineered cells, and wherein the first population of engineered cells do not induce an immune response to the cell upon administration to the recipient patient.

344. The method of any one of Items 1-339, wherein the therapeutic agent comprises a first population of engineered cells, and wherein the first population of engineered cells persist in the patient for at least 4 weeks, at least 2 months, at least 3 months, at least 4 months, at least 5 months, at least 6 months, at least 7 months, at least 8 months, at least 9 months, at least 10 months, at least 11 months, at least 12 months, or longer.

345. The method of any one of Items 1-339, wherein the therapeutic agent comprises a first population of engineered cells, wherein the one or more targeted therapies comprise an autologous or allogeneic cell-based therapy, and wherein the first population of engineered cells persist in the patient for longer than cells of the one or more targeted therapies.

346. The method of any one of Items 340-345, wherein the therapeutic effect of the first population of engineered cells lasts for a duration of at least 4 weeks, at least 2 months, at least 3 months, at least 4 months, at least 5 months, at least 6 months, at least 7 months, at least 8 months, at least 9 months, at least 10 months, at least 11 months, at least 12 months, or longer.

347. The method of any one of Items 340-346, wherein the therapeutic effect of the first population of engineered cells lasts for longer than that of the one or more targeted therapies.

348. Use of therapeutic agent directed to a first therapeutic target for treating a disease or disorder in a patient.

349. The use of Item 348, wherein the therapeutic agent is further directed to a second therapeutic target, wherein the first therapeutic target and the second therapeutic target are different.

350. The use of Item 348, wherein the patient has previously been administered one or more targeted therapies directed to a second therapeutic target, wherein the first therapeutic target and the second therapeutic target are different.

351. The use of any one of Items 348-350, wherein the patient has not previously been administered targeted therapies for the treatment of the disease or disorder.

352. The use of Item 348, wherein the patient has previously been administered one or more targeted therapies directed to a second therapeutic target, wherein the therapeutic agent is further directed to a second therapeutic target, and wherein the first therapeutic target and the second therapeutic target are different.

353. The use of any one of Items 348-352, wherein the patient has not previously received a therapy directed to the first therapeutic target.

354. The use of any one of Items 349-353, wherein the patient has not previously received a therapy directed to the second therapeutic target.

355. The use of Item 348, wherein the patient is at risk of antigen evasion, and wherein the therapeutic agent is directed to the first therapeutic target and a second therapeutic target, wherein the first therapeutic target and the second therapeutic target are different therapeutic targets.

356. The use of Item 348, wherein the patient has previously been administered one or more targeted therapies directed to a second therapeutic target, wherein the therapeutic agent comprises a population of engineered CAR-T cells, wherein the engineered CAR-T cells of the population comprise one or more chimeric antigen receptors (CARs), wherein at least one CAR is directed to the first therapeutic target, and wherein the first therapeutic target and the second therapeutic target are different.

357. The use of Item 348, wherein the disease or disorder is characterized by antigen evasion, wherein the patient has previously been administered one or more targeted therapies directed to a second therapeutic target, wherein the therapeutic agent comprises a population of engineered CAR-T cells, wherein the engineered CAR-T cells of the population comprise one or more chimeric antigen receptors (CARs), wherein at least one CAR is directed to the first therapeutic target, and wherein the first therapeutic target and the second therapeutic target are different.

358. The use of any one of Items 349-354, wherein the patient is at risk of antigen evasion, wherein the therapeutic agent comprises a population of engineered CAR-T cells, wherein the engineered CAR-T cells of the population comprise one or more chimeric antigen receptors (CARs), wherein at least one CAR is directed to the first therapeutic target, and wherein the first therapeutic target and the second therapeutic target are different.

359. The use of Item 348, wherein the patient is at risk of antigen evasion, wherein the patient has previously been administered one or more targeted therapies directed to a second therapeutic target, wherein the therapeutic agent comprises a first population of engineered CAR-T cells, wherein the engineered CAR-T cells of the first population comprise one or more chimeric antigen receptors (CARs), wherein at least one CAR is directed to the first therapeutic target, and wherein the first therapeutic target and the second therapeutic target are different.

360. The use of any one of Items 348-359, wherein the first therapeutic target is a first antigen.

361. The use of Item 360, wherein the first antigen is an antigen associated with the disease or the disorder.

362. The use of Item 360 or 361, wherein the first antigen is an antigen present on the surface of a B cell.

363. The use of Item 362, wherein the B cell is a malignant B cell.

364. The use of any one of Items 360-362, wherein the first antigen is CD22, CD20, CD19, BCMA, GPRC5D, CD38, CD70, CD79b, HER2, IL13Ra2, MUC1, or a variant thereof.

365. The use of any one of Items 360-364, wherein the first antigen is CD22, CD20, CD19, BCMA, GPRC5D, CD38, CD70, CD79b, HER2, IL13Ra2, or MUC1.

366. The use of any one of Items 349-365, wherein the second therapeutic target is a second antigen.

367. The use of Item 366, wherein the second antigen is an antigen associated with the disease or the disorder.

368. The use of Item 366 or 367, wherein the second antigen is an antigen present on the surface of a B cell.

369. The use of Item 368, wherein the B cell is a malignant B cell.

370. The use of any one of Items 366-369, wherein the second antigen is CD22, CD20, CD19, BCMA, GPRC5D, CD38, CD70, CD79b, HER2, IL13Ra2, MUC1, or a variant thereof.

371. The use of any one of Items 366-370, wherein the second antigen is CD22, CD20, CD19, BCMA, GPRC5D, CD38, CD70, CD79b, HER2, IL13Ra2, or MUC1.

372. The use of any one of Items 366-371, wherein the second antigen is CD22, CD20, or CD19.

373. The use of any one of Items 349-372, wherein the therapeutic agent comprises a first immunotherapeutic agent.

374. The use of any one of Items 349-373, wherein the therapeutic agent comprises first population of engineered cells.

375. The use of Item 374, wherein the first population of engineered cells comprises engineered cells directed to the first therapeutic target.

376. The use of Item 374 or 375, wherein the first population of engineered cells comprises engineered cells that comprise a first immunotherapeutic agent.

377. The use of Item 373 or 376, wherein the first immunotherapeutic agent comprises a first antigen binding domain.

378. The use of any one of Items 373, 376, and 377, wherein the first immunotherapeutic agent comprises an antibody, a Fab, an scFV, an scFV-Fc, an scFV zipper, a diabody, a minibody, a CAR, a CAAR, a CAAR-T cell, a BAR, or a BAR-T cell.

379. The use of any one of Items 374-378, wherein the first population of engineered cells is a first population of engineered CAR-T cells.

380. The use of Item 379, wherein the first population of engineered CAR-T cell comprises one or more chimeric antigen receptors (CARs),
wherein at least one CAR comprises
(i) is directed to the first therapeutic target, and
(ii) comprises a first antigen binding domain.

381. The use of Item 379 or 380, wherein at least one CAR of the first population of engineered CAR-T cells,
(i) is directed to the second therapeutic target and
(ii) comprises a second antigen binding domain.

382. The use of any one of Items 373-381, wherein the therapeutic agent further comprises a second immunotherapeutic agent.

383. The use of any one of Items 373-382, wherein the therapeutic agent further comprises a second population of engineered cells.

384. The use of Item 383, wherein the second population of engineered cells comprises engineered cells directed to the second therapeutic target.

385. The use of Item 383 or 384, wherein the second population of engineered cells comprises engineered cells that comprise a second immunotherapeutic agent.

386. The use of Item 385, wherein the second immunotherapeutic agent comprises a second antigen binding domain.

387. The use of Item 385 or 389, wherein the second immunotherapeutic agent comprises an antibody, a Fab, an scFV, an scFV-Fc, an scFV zipper, a diabody, a minibody, a CAR, a CAAR, a CAAR-T cell, a BAR, or a BAR-T cell.

388. The use of Item 383-387, wherein the second population of engineered cells is a second population of engineered CAR-T cell.

389. The use of ant one of Items 383-388, wherein the therapeutic agent comprises a second population of engineered CAR-T cells,
wherein the second population of engineered CAR-T cells comprise one or more chimeric antigen receptors (CARs),
wherein at least one CAR comprises a second antigen binding domain.

390. The use of any one of Items 348-373, wherein the therapeutic agent comprises one or more populations of engineered CAR-T cells.

391. The use of Item 390, wherein the therapeutic agent comprises the first population of engineered CAR-T cells and the second population of engineered CAR-T cells,
wherein the first population of engineered CAR-T cells comprise one or more chimeric antigen receptors (CARs), wherein at least one CAR of the first population of engineered CAR-T cells
(i) is directed to the first therapeutic target, and
(ii) comprises the first antigen binding domain, and
wherein the second population of engineered CAR-T cells comprise one or more chimeric antigen receptors (CARs), wherein at least one CAR of the second population of engineered CAR-T cell
(i) is directed to the second therapeutic target, and
(ii) comprises the second antigen binding domain.

392. The use of any one of Items 348-373, wherein the therapeutic agent comprises a first population of engineered CAR-T cells and a second population of engineered CAR-T cells,
wherein the first population of engineered CAR-T cells comprise one or more chimeric antigen receptors (CARs), wherein at least one CAR of the first population of engineered CAR-T cells,
(i) is directed to the first therapeutic target, and
(ii) comprises a first antigen binding domain,
wherein the second population of engineered CAR-T cells comprise two or more chimeric antigen receptors (CARs), wherein at least one CAR of the second population of engineered CAR-T cells,
(i) is directed to the first therapeutic target, and
(ii) comprises a first antigen binding domain, and
wherein at least one CAR of the second population of engineered CAR-T cells,
(i) is directed to the second therapeutic target and
(ii) comprises a second antigen binding domain.

393. The use of any one of Items 348-373, wherein the therapeutic agent comprises a first population of engineered CAR-T cells, a second population of engineered CAR-T cells, and a third population of engineered CAR-T cells,
wherein the first population of engineered CAR-T cells comprise one or more chimeric antigen receptors (CARs), wherein at least one CAR of the first population of engineered CAR-T cells,
(i) is directed to the first therapeutic target, and
(ii) comprises a first antigen binding domain,
wherein the second population of engineered CAR-T cells comprise one or more chimeric antigen receptors (CARs), wherein at least one CAR of the second population of engineered CAR-T cells
(iii) is directed to the second therapeutic target, and
(iv) comprises a second antigen binding domain, and
wherein the third population of engineered CAR-T cells comprise two or more chimeric antigen receptors (CARs), wherein at least one CAR of the third population of engineered CAR-T cells
(v) is directed to the first therapeutic target, and
(vi) comprises a first antigen binding domain, and
wherein at least one CAR of the third population of engineered CAR-T cells
(vii) is directed to the second therapeutic target and
(viii) comprises a second antigen binding domain.

394. The use of any one of Items 377-393, wherein the first antigen binding domain is capable of binding to CD22 or variant thereof.

395. The use of any one of Items 377-394, wherein the first antigen binding domain is capable of binding to CD22.

396. The use of any one of Items 377-395, wherein the first antigen binding domain comprises a heavy chain complementarity determining region 1 (HCDR1) comprising an amino acid sequence according to SEQ ID NO: 47, a heavy chain complementarity determining region 2 (HCDR2) comprising an amino acid sequence according to SEQ ID NO: 48, and a heavy chain complementarity determining region 3 (HCDR3) comprising an amino acid sequence according to SEQ ID NO: 49.

397. The use of any one of Items 377-396, wherein the first antigen binding domain comprises a light chain complementarity determining region 1 (LCDR1) comprising an amino acid sequence according to SEQ ID NO: 51, a light chain complementarity determining region 2 (LCDR2) comprising an amino acid sequence according to SEQ ID NO: 52, and a light chain complementarity determining region 3 (LCDR3) comprising an amino acid sequence according to SEQ ID NO: 53.

398. The use of any one of Items 377-395, wherein the first antigen binding domain comprises a heavy chain variable domain (VH) comprising an amino acid sequence according to SEQ ID NO: 47, an amino acid sequence according to SEQ ID NO: 48, and an amino acid sequence according to SEQ ID NO: 49 arranged non-contiguously from N-terminus to C-terminus.

399. The use of any one of Items 377-397, wherein the first antigen binding domain comprises a heavy chain variable domain (VH) comprising an amino acid sequence that is at least 80% identical, at least 80%, at least 85%, at least 90%, at least 95%, at least 96%, at least 97%, at least 98%, at least 99%, or 100% identical to an amino acid sequence according to SEQ ID NO: 46.

400. The use of any one of Items 377-395 and 398, wherein the first antigen binding domain comprises a light chain variable domain (VL) comprising an amino acid sequence according to SEQ ID NO: 51, an amino acid sequence according to SEQ ID NO: 52, and an amino acid sequence according to SEQ ID NO: 53 arranged non-contiguously from N-terminus to C-terminus.

401. The use of any one of Items 377-397 and 399, wherein the first antigen binding domain comprises a light chain variable domain (VL) comprising an amino acid sequence that is at least 80% identical, at least 80%, at least 85%, at least 90%, at least 95%, at least 96%, at least 97%, at least 98%, at least 99%, or 100% identical to an amino acid sequence according to SEQ ID NO: 50.

402. The use of any one of Items 377-395, wherein the first antigen binding domain comprises an HCDR1 according to SEQ ID NO: 56, an HCDR2 according to SEQ ID NO: 57, and an HCDR3 according to SEQ ID NO: 58.

403. The use of any one of Items 377-395 and 402, wherein the first antigen binding domain comprises an LCDR1 according to SEQ ID NO: 60, an LCDR2 according to SEQ ID NO: 61, and an LCDR3 according to SEQ ID NO: 62.

404. The use of any one of any one of Items 377-395, wherein the first antigen binding domain comprises a heavy chain variable domain (VH) comprising an amino acid sequence according to SEQ ID NO: 56, an amino acid sequence according to SEQ ID NO: 57, and an amino acid sequence according to SEQ ID NO: 58 arranged non-contiguously from N-terminus to C-terminus.

405. The use of any one of Items 377-395, 402 and 403, wherein the first antigen binding domain comprises a heavy chain variable domain (VH) comprising an amino acid sequence that is at least 80% identical, at least 80%, at least 85%, at least 90%, at least 95%, at least 96%, at least 97%, at least 98%, at least 99%, or 100% identical to an amino acid sequence according to SEQ ID NO: 55.

406. The use of any one of Items 377-395 and 404, wherein the first antigen binding domain a light chain variable domain (VL) comprising comprises an amino acid sequence according to SEQ ID NO: 60, an amino acid sequence according to SEQ ID NO: 61, and an amino acid sequence according to SEQ ID NO: 62 arranged non-contiguously from N-terminus to C-terminus.

407. The use of any one of Items 377-395, 402, 403 and 405 wherein the first antigen binding domain comprises a light chain variable domain (VL) comprising an amino acid sequence that is at least 80% identical, at least 80%, at least 85%, at least 90%, at least 95%, at least 96%, at least 97%, at least 98%, at least 99%, or 100% identical to an amino acid sequence according to SEQ ID NO: 59.

408. The use of any one of Items 377-395, wherein the first antigen binding domain is capable of binding to CD19 or variant thereof.

409. The use of any one of Items 377-395 and 408, wherein the first antigen binding domain is capable of binding to CD19.

410. The use of any one of Items 377-395, 408 and 409, wherein the first antigen binding domain comprises an HCDR1 according to SEQ ID NO: 26, an HCDR2 according to SEQ ID NO: 27, and an HCDR3 according to SEQ ID NO: 28.

411. The use of any one of Items 377-395, and 408-410, wherein the first antigen binding domain comprises an LCDR1 according to SEQ ID NO: 21, an LCDR2 according to SEQ ID NO: 22, and an LCDR3 according to SEQ ID NO: 23.

412. The use of any one of Items 377-395, 408 and 409, wherein the first antigen binding domain comprises a heavy chain variable domain (VH) comprising an amino acid sequence according to SEQ ID NO: 26, an amino acid sequence according to SEQ ID NO: 27, and an amino acid sequence according to SEQ ID NO: 28 arranged non-contiguously from N-terminus to C-terminus.

413. The use of any one of Items 377-395, and 408-411, wherein the first antigen binding domain comprises a heavy chain variable domain (VH) comprising an amino acid sequence that is at least 80% identical, at least 80%, at least 85%, at least 90%, at least 95%, at least 96%, at least 97%, at least 98%, at least 99%, or 100% identical to an amino acid sequence according to SEQ ID NO: 25.

414. The use of any one of Items 377-395, 408, 409 and 412, wherein the first antigen binding domain comprises a light chain variable domain (VL) comprising an amino acid sequence according to SEQ ID NO: 21, an amino acid sequence according to SEQ ID NO: 22, and an amino acid sequence according to SEQ ID NO: 23 arranged non-contiguously from N-terminus to C-terminus.

415. The use of any one of Items 377-395, 408-411 and 413, wherein the first antigen binding domain comprises a light chain variable domain (VL) comprising an amino acid sequence that is at least 80% identical, at least 80%, at least 85%, at least 90%, at least 95%, at least 96%, at least 97%, at least 98%, at least 99%, or 100% identical to an amino acid sequence according to SEQ ID NO: 20.

416. The use of any one of Items 377-395, wherein the first antigen binding domain is capable of binding to CD20 or variant thereof.

417. The use of any one of Items 377-395 and 416, wherein the first antigen binding domain is capable of binding to CD20.

418. The use of any one of Items 377-395, 416 and 417, wherein the first antigen binding domain comprises an HCDR1 according to SEQ ID NO: 43, an HCDR2 according to SEQ ID NO: 44, and an HCDR3 according to SEQ ID NO: 107.

419. The use of any one of Items 377-395 and 416-418, wherein the first antigen binding domain comprises an LCDR1 according to SEQ ID NO: 39, an LCDR2 according to SEQ ID NO: 40, and an LCDR3 according to SEQ ID NO: 41.

420. The use of any one of Items 377-395, 416 and 417, wherein the first antigen binding domain comprises a heavy chain variable domain (VH) comprising an amino acid sequence according to SEQ ID NO: 43, an amino acid sequence according to SEQ ID NO: 44, and an amino acid sequence according to SEQ ID NO: 107 arranged non-contiguously from N-terminus to C-terminus.

421. The use of any one of Items 377-395 and 416-419, wherein the first antigen binding domain comprises a heavy chain variable domain (VH) comprising an amino acid sequence that is at least 80% identical, at least 80%, at least 85%, at least 90%, at least 95%, at least 96%, at least 97%, at least 98%, at least 99%, or 100% identical to an amino acid sequence according to SEQ ID NO: 42.

422. The use of any one of Items 377-395, 416, 417 and 420, wherein the first antigen binding domain comprises a light chain variable domain (VL) comprising an amino acid sequence according to SEQ ID NO: 39, an amino acid sequence according to SEQ ID NO: 40, and an amino acid sequence according to SEQ ID NO: 41 arranged non-contiguously from N-terminus to C-terminus.

423. The use of any one of Items 377-395, 416-419 and 421, wherein the first antigen binding domain comprises a light chain variable domain (VL) comprising an amino acid sequence that is at least 80% identical, at least 80%, at least 85%, at least 90%, at least 95%, at least 96%, at least 97%, at least 98%, at least 99%, or 100% identical to an amino acid sequence according to SEQ ID NO: 38.

424. The use of any one of Items 386-389, wherein the second antigen is CD19 or a variant thereof.

425. The use of any one of Items 386-389 and 424, wherein the second antigen is CD19.

426. The use of any one of Items 386-389, 424 and 425, wherein the second antigen binding domain comprises an HCDR1 according to SEQ ID NO: 26, an HCDR2 according to SEQ ID NO: 27, and an HCDR3 according to SEQ ID NO: 28.

427. The use of any one of Items 386-389 and 424-426, wherein the second antigen binding domain comprises an LCDR1 according to SEQ ID NO: 21, an LCDR2 according to SEQ ID NO: 22, and an LCDR3 according to SEQ ID NO: 23.

428. The use of any one of any one of Items 386-389, 424 and 425, wherein the first antigen binding domain comprises a heavy chain variable domain (VH) comprising an amino acid sequence according to SEQ ID NO: 27, an amino acid sequence according to SEQ ID NO: 28, and an amino acid sequence according to SEQ ID NO: 29 arranged non-contiguously from N-terminus to C-terminus.

429. The use of any one of Items 386-389 and 424-427, wherein the second antigen binding domain comprises a heavy chain variable domain (VH) comprising an amino acid sequence that is at least 80% identical, at least 80%, at least 85%, at least 90%, at least 95%, at least 96%, at least 97%, at least 98%, at least 99%, or 100% identical to an amino acid sequence according to SEQ ID NO: 25.

430. The use of any one of Items 386-389, 424, 425 and 428, wherein the first antigen binding domain comprises a light chain variable domain (VL) comprising an amino acid sequence according to SEQ ID NO: 21, an amino acid sequence according to SEQ ID NO: 22, and an amino acid sequence according to SEQ ID NO: 23 arranged non-contiguously from N-terminus to C-terminus.

431. The use of any one of Items 386-389, 424-427 and 429, wherein the second antigen binding domain comprises a light chain variable domain (VL) comprising an amino acid sequence that is at least 80% identical, at least 80%, at least 85%, at least 90%, at least 95%, at least 96%, at least 97%, at least 98%, at least 99%, or 100% identical to an amino acid sequence according to SEQ ID NO: 20.

432. The use of any one of Items 386-389, wherein the second antigen is CD20 or a variant thereof.

433. The use of any one of Items 386-389 and 432, wherein the second antigen is CD20.

434. The use of any one of Items 386-389, 432 and 433, wherein the second antigen binding domain comprises an HCDR1 according to SEQ ID NO: 43, an HCDR2 according to SEQ ID NO: 44, and an HCDR3 according to SEQ ID NO: 107.

435. The use of any one of Items 386-389, and 432-444, wherein the second antigen binding domain comprises an LCDR1 according to SEQ ID NO: 39, an LCDR2 according to SEQ ID NO: 40, and an LCDR3 according to SEQ ID NO: 41.

436. The use of any one of Items 386-389, 432 and 433, wherein the first antigen binding domain comprises a heavy chain variable domain (VH) comprising an amino acid sequence according to SEQ ID NO: 43, an amino acid sequence according to SEQ ID NO: 44, and an amino acid sequence according to SEQ ID NO: 107 arranged non-contiguously from N-terminus to C-terminus.

437. The use of any one of Items 386-389 and 432-435, wherein the second antigen binding domain comprises a heavy chain variable domain (VH) comprising an amino acid sequence that is at least 80% identical, at least 80%, at least 85%, at least 90%, at least 95%, at least 96%, at least 97%, at least 98%, at least 99%, or 100% identical to an amino acid sequence according to SEQ ID NO: 42.

438. The use of any one of Items 386-389, 432, 433 and 436, wherein the first antigen binding domain comprises a light chain variable domain (VL) comprising an amino acid sequence according to SEQ ID NO: 39, an amino acid sequence according to SEQ ID NO: 40, and an amino acid sequence according to SEQ ID NO: 41 arranged non-contiguously from N-terminus to C-terminus.

439. The use of any one of Items 386-389, 432-435 and 437, wherein the second antigen binding domain comprises a light chain variable domain (VL) comprising an amino acid sequence that is at least 80% identical, at least 80%, at least 85%, at least 90%, at least 95%, at least 96%, at least 97%, at least 98%, at least 99%, or 100% identical to an amino acid sequence according to SEQ ID NO: 38.

440. The use of any one of Items 386-389, wherein the second antigen is CD22 or a variant thereof.

441. The use of any one of Items 386-389 and 440, wherein the second antigen is CD22.

442. The use of any one of Items 386-389, 440 and 441, wherein the second antigen binding domain comprises an HCDR1 according to SEQ ID NO: 47, an HCDR2 according to SEQ ID NO: 48, and an HCDR3 according to SEQ ID NO: 49.

443. The use of any one of Items 386-389 and 440-442, wherein the second antigen binding domain comprises an LCDR1 according to SEQ ID NO: 51, an LCDR2 according to SEQ ID NO: 52, and an LCDR3 according to SEQ ID NO: 53.

444. The use of any one of Items 386-389, 440 and 441, wherein the first antigen binding domain comprises a heavy chain variable domain (VH) comprising an amino acid sequence according to SEQ ID NO: 47, an amino acid sequence according to SEQ ID NO: 48, and an amino acid sequence according to SEQ ID NO: 49 arranged non-contiguously from N-terminus to C-terminus.

445. The use of any one of Items 386-389 and 440-443, wherein the second antigen binding domain comprises a heavy chain variable domain (VH) comprising an amino acid sequence that is at least 80% identical, at least 80%, at least 85%, at least 90%, at least 95%, at least 96%, at least 97%, at least 98%, at least 99%, or 100% identical to an amino acid sequence according to SEQ ID NO: 46].

446. The use of any one of Items 386-389, 440, 441 and 444, wherein the first antigen binding domain comprises a light chain variable domain (VL) comprising an amino acid sequence according to SEQ ID NO: 51, an amino acid sequence according to SEQ ID NO: 52, and an amino acid sequence according to SEQ ID NO: 53 arranged non-contiguously from N-terminus to C-terminus.

447. The use of any one of Items 386-389, 440-443 and 445, wherein the second antigen binding domain comprises a light chain variable domain (VL) comprising an amino acid sequence that is at least 80% identical, at least 80%, at least 85%, at least 90%, at least 95%, at least 96%, at least 97%, at least 98%, at least 99%, or 100% identical to an amino acid sequence according to SEQ ID NO: 50.

448. The use of any one of Items 350-447, wherein the one or more targeted therapies comprise a fourth immunotherapeutic agent.

449. The use of any one of Items 350-448, wherein the one or more targeted therapies comprise a fourth population of engineered cells.

450. The use of Item 449, wherein the fourth population of engineered cells is directed to the second therapeutic target.

451. The use of Item 449 or 450, wherein the second population of engineered cells and the fourth population of engineered cells comprise engineered cells that are directed to the same therapeutic target.

452. The use of any one of Items 449-451, wherein the second population of engineered cells and the fourth population of engineered cells are directed to different therapeutic targets.

453. The use of any one of Items 449-452, wherein the fourth population of engineered cells comprises a fourth immunotherapeutic agent.

454. The use of Item 453, wherein the second immunotherapeutic agent and the fourth immunotherapeutic agent are the same.

455. The use of Item 453, wherein the second immunotherapeutic agent and the fourth immunotherapeutic agent are different.

456. The use of any one of Items 453-455, wherein the fourth immunotherapeutic agent comprises a fourth antigen binding domain.

457. The use of Item 456, wherein the second antigen binding domain and the fourth antigen binding domain are the same.

458. The use of Item 456, wherein the second antigen binding domain and the fourth antigen binding domain are different.

459. The use of any one of Items 453-458, wherein the fourth immunotherapeutic agent comprises an antibody, a Fab, an scFV, an scFV-Fc, an scFV zipper, a diabody, a minibody, a CAR, a CAAR, a CAAR-T cell, a BAR, or a BAR-T cell.

460. The use of any one of Items 453-459, wherein the fourth population of engineered cells is a fourth population of engineered CAR-T cells.

461. The use of Item 460, wherein the second population of engineered CAR-T cells and the fourth population of engineered CAR-T cells comprise engineered CAR-T cells that are directed to the same therapeutic target.

462. The use of Item 460 or 461, wherein the second population of engineered CAR-T cells and the fourth population of engineered CAR-T cells comprise engineered CAR-T cells that are directed to the same therapeutic target.

463. The use of any one of Items 460-462, wherein the one or more targeted therapies comprises a fourth population of engineered CAR-T cells, wherein the fourth population of engineered CAR-T cells comprises one or more chimeric antigen receptors (CARs), wherein at least one CAR comprises a fourth antigen binding domain.

464. The use of any one of Items 350-354, and 355-463, wherein the one or more targeted therapies comprise a failed therapy.

465. The use of Item 464, wherein the failed therapy is characterized by one or more of: (a) a plateau or increase in one or more symptom of the disease, (b) a plateau or a worsening of the extent or state of the disease, (c) a plateau or a worsening of disease progression, (d) an attenuated response to therapy, and (e) disease recurrence.

466. The use of any one of Items 456-465, wherein the fourth antigen binding domain comprises an HCDR1 according to SEQ ID NO: 26, an HCDR2 according to SEQ ID NO: 27, and an HCDR3 according to SEQ ID NO: 28.

467. The use of any one of Items 456-466, wherein the fourth antigen binding domain comprises an LCDR1 according to SEQ ID NO: 21, an LCDR2 according to SEQ ID NO: 22, and an LCDR3 according to SEQ ID NO: 23.

468. The use of any one of Items 456-467, wherein the fourth antigen binding domain comprises a heavy chain variable domain (VH) comprising an amino acid sequence that is at least 80% identical, at least 80%, at least 85%, at least 90%, at least 95%, at least 96%, at least 97%, at least 98%, at least 99%, or 100% identical to an amino acid sequence according to SEQ ID NO: 25.

469. The use of any one of Items 456-468, wherein the fourth antigen binding domain comprises a light chain variable domain (VL) comprising an amino acid sequence that is at least 80% identical, at least 80%, at least 85%, at least 90%, at least 95%, at least 96%, at least 97%, at least 98%, at least 99%, or 100% identical to an amino acid sequence according to SEQ ID NO: 20.

470. The use of any one of Items 456-465, wherein the fourth antigen binding domain comprises an HCDR1 according to SEQ ID NO: 43, an HCDR2 according to SEQ ID NO: 44, and an HCDR3 according to SEQ ID NO: 107.

471. The use of any one of Items 456-465 and 470, wherein the fourth antigen binding domain comprises an LCDR1 according to SEQ ID NO: 39, an LCDR2 according to SEQ ID NO: 40, and an LCDR3 according to SEQ ID NO: 41.

472. The use of any one of Items 456-465, 470 and 471, wherein the fourth antigen binding domain comprises a heavy chain variable domain (VH) comprising an amino acid sequence that is at least 80% identical, at least 80%, at least 85%, at least 90%, at least 95%, at least 96%, at least 97%, at least 98%, at least 99%, or 100% identical to an amino acid sequence according to SEQ ID NO: 42.

473. The use of any one of Items 456-465, and 470-472, wherein the fourth antigen binding domain comprises a light chain variable domain (VL) comprising an amino acid sequence that is at least 80% identical, at least 80%, at least 85%, at least 90%, at least 95%, at least 96%, at least 97%, at least 98%, at least 99%, or 100% identical to an amino acid sequence according to SEQ ID NO: 38.

474. The use of any one of Items 456-465, wherein the fourth antigen binding domain comprises an HCDR1 according to SEQ ID NO: 47, an HCDR2 according to SEQ ID NO: 48, and an HCDR3 according to SEQ ID NO: 49.

475. The use of any one of Items 456-465 and 474, wherein the fourth antigen binding domain comprises an LCDR1 according to SEQ ID NO: 51, an LCDR2 according to SEQ ID NO: 52, and an LCDR3 according to SEQ ID NO: 53.

476. The use of any one of Items 456-465, 474 and 475, wherein the fourth antigen binding domain comprises a heavy chain variable domain (VH) comprising an amino acid sequence that is at least 80% identical, at least 80%, at least 85%, at least 90%, at least 95%, at least 96%, at least 97%, at least 98%, at least 99%, or 100% identical to an amino acid sequence according to SEQ ID NO: 46.

477. The use of any one of Items 456-465, 474-476, wherein the fourth antigen binding domain comprises a light chain variable domain (VL) comprising an amino acid sequence that is at least 80% identical, at least 80%, at least 85%, at least 90%, at least 95%, at least 96%, at least 97%, at least 98%, at least 99%, or 100% identical to an amino acid sequence according to SEQ ID NO: 50.

478. The use of any one of Items 348-477, wherein the patient is at risk of antigen evasion.

479. The use of any one of Items 348-478, wherein the patient is suspected of having antigen evasion.

480. The use of any one of Items 348-479, wherein the patient is at risk of antigen drift.

481. The use of any one of Items 348-480, wherein the patient is suspected of having antigen drift.

482. The use of any one of Items 348-481, wherein the patient is at risk of or suffering from cancer.

483. The use of any one of Items 348-482, wherein the cancer is a B cell malignancy.

484. The use of any one of Items 348-483, wherein the disease or disorder is characterized by antigen evasion.

485. The use of any one of Items 348-484, wherein the disease or disorder is prone to antigen evasion.

486. The use of any one of Items 348-485, wherein the disease or disorder is characterized by antigenic drift.

487. The use of any one of Items 348-486, wherein the disease or disorder is prone to antigenic drift.

488. The use of any one of Items 348-487, wherein the disease or disorder is cancer.

489. The use of Item 488, wherein the cancer is or comprises lymphoma, leukemia, B-cell acute lymphoblastic leukemia (B-ALL), B-cell Non-Hodgkin lymphoma (B-NHL), or B-cell chronic lymphoblastic leukemia.

490. The use of Item 488 or 489, wherein the cancer is or comprises lymphoma.

491. The use of Item 490, wherein the lymphoma is a B cell lymphoma.

492. The use of Item 488 or 489, wherein the cancer is or comprises leukemia.

493. The use of Item 488 or 489, wherein the cancer is or comprises B-cell acute lymphoblastic leukemia (B-ALL).

494. The use of Item 488 or 489, wherein the cancer is or comprises B-cell Non-Hodgkin lymphoma (B-NHL).

495. The use of Item 488 or 489, wherein the cancer is or comprises B-cell chronic lymphoblastic leukemia.

496. The use of Item 488 or 489, wherein the cancer comprises a B cell malignancy.

497. The use of any one of Items 393, 396-401, 440-496, wherein engineered CAR-T cells of the first, second, and/or third population comprise one or more CARs comprising a leader sequence, CD8α signal peptide, a linker, an m971 binder-based scFv, a CD8α hinge domain, a CD8 transmembrane domain, a CD28 transmembrane domain, a 4-1BB costimulatory domain, a CD28 signaling domain, a CD137 signaling domain, a CD8 signaling domain, a CD3ζ signaling domain, or a combination thereof.

498. The use of any one of Items 393, 396-401, 440-497, wherein engineered CAR-T cells of the first, second, and/or third population comprise one or more CARs comprise one or more CARs comprise a CD8α transmembrane domain or a CD28 transmembrane domain.

499. The use of any one of Items 393, 396-401, 440-498, wherein engineered CAR-T cells of the first, second, and/or third population comprise one or more CARs comprise one or more CARs comprise a CD137 signaling domain and a CD3ζ signaling domain.

500. The use of any one of Items 393, 396-401, 440-499, wherein engineered CAR-T cells of the first, second, and/or third population comprise one or more CARs comprise one or more CARs comprise a CD28 signaling domain and a CD3ζ signaling domain.

501. The use of any one of Item 497-500, wherein engineered CAR-T cells of the first, second, and/or third population comprise one or more CARs comprise one or more CARs comprise a CD28 signaling domain, a CD137 signaling domain, and a CD3ζ signaling domain.

502. The use of any one of Item 497-501, wherein the CD8α signal peptide comprises an amino acid sequence according to SEQ ID NO: 6.

503. The use of any one of Item 497-502, wherein the linker is selected from the group consisting of IgG linkers, Whitlow linkers, (G4S)n linkers, wherein n is 1, 2, 3, 4, or more, and modifications thereof.

504. The use of any one of Item 497-503, wherein the linker is a (G4S)n linker, wherein n is 1 or 3.

505. The use of any one of Item 497-504, wherein the CD8α hinge domain comprises an amino acid sequence according to SEQ ID NO: 9.

506. The use of any one of Item 497-505, wherein the CD8 transmembrane domain comprises an amino acid sequence according to SEQ ID NO: 14 or 86.

507. The use of any one of Item 497-506, wherein the CD28 transmembrane domain comprises an amino acid sequence according to SEQ ID NO: 15, 87, or 114.

508. The use of any one of Item 497-507, wherein the 4-1BB costimulatory domain comprises an amino acid sequence according to SEQ ID NO: 16.

509. The use of any one of Item 497-508, wherein the CD28 signaling domain comprises an amino acid sequence according to SEQ ID NO: 17 or 88.

510. The use of any one of Item 497-509, wherein the CD137 signaling domain comprises an amino acid sequence according to SEQ ID NO: 90.

511. The use of any one of Item 497-510, wherein the CD8 signaling domain comprises an amino acid sequence according to SEQ ID NO: 89.

512. The use of any one of Item 497-511, wherein the CD3ζ signaling domain comprises an amino acid sequence according to SEQ ID NO: 18 or 115.

513. The use of Item 393, 396-401, 440-512, wherein engineered CAR-T cells of the first, second, and/or third population comprise one or more CARs comprise one or more CARs comprise an amino acid sequence that is at least 80%, at least 85%, at least 90%, at least 95%, at least 96%, at least 97%, at least 98%, at least 99%, or 100% identical to an amino acid sequence according to SEQ ID NO: 91, 92, or 93.

514. The use of any one of the preceding Items, wherein engineered CAR-T cells of the first, second, and/or third population are propagated from a primary T cell or a progeny thereof, or are derived from a T cell differentiated from an iPSC or a progeny thereof.

515. The use of any one of the preceding Items, wherein engineered CAR-T cells of the first, second, and/or third population are differentiated cells derived from an induced pluripotent stem cell or a progeny thereof.

516. The use of any one of the preceding Items, wherein engineered CAR-T cells of the first, second, and/or third population are a progeny of primary immune cells.

517. The use of any one of the preceding Items, wherein engineered CAR-T cells of the first, second, and/or third population are a CAR+ T cell, a CD4+ CAR+ T cell, or a CD8+ CAR+ T cell.

518. The use of any one of the preceding Items, wherein engineered CAR-T cells of the first, second, and/or third population are autologous CAR-T cells.

519. The use of any one of the preceding Items, wherein engineered CAR-T cells of the first, second, and/or third population are allogeneic CAR-T cells.

520. The use of any one of the preceding Items, wherein engineered CAR-T cells of the first, second, and/or third population are primary cells.

521. The use of any one of the preceding Items, wherein the primary cells are derived from a single donor.

522. The use of any one of the preceding Items, wherein the primary cells are derived from two or more donors.

523. The use of any one of the preceding Items, wherein engineered CAR-T cells of the first, second, and/or third population are derived from induced pluripotent stem cells (iPSCs).

524. The use of any one of the preceding Items, wherein the iPSCs are derived from a single donor.

525. The use of any one of the preceding Items, wherein the iPSCs are derived from two or more donors.

526. The use of any one of the preceding Items, wherein engineered cells (e.g., engineered CAR-T cells) of the first, second, and/or third population comprise reduced expression of a functional major histocompatibility complex class I human leukocyte antigen (HLA-1) complex relative to an unaltered or unmodified wild-type or control cell.

527. The use of any one of the preceding Items, wherein engineered cells (e.g., engineered CAR-T cells) of the first, second, and/or third population comprise one or more genetic modifications that reduce expression of one or more HLA-I molecules or HLA I associated molecules relative to an unaltered or unmodified wild-type or control cell.

528. The use of any one of the preceding Items, wherein engineered cells (e.g., engineered CAR-T cells) of the first, second, and/or third population do not express one or more HLA-I molecules or HLA I associated molecules.

529. The use of any one of the preceding Items, wherein the one or more HLA-I molecules comprise HLA-A, HLA-B, HLA-C, or a combination thereof.

530. The use of any one of the preceding Items, wherein the one or more HLA-I molecules comprise HLA-A.

531. The use of any one of the preceding Items, wherein the one or more HLA-I molecules comprise HLA-B.

532. The use of any one of the preceding Items, wherein the one or more HLA-I molecules comprise HLA-C.

533. The use of any one of the preceding Items, wherein the one or more HLA-I associated molecules comprise β-2 microglobulin (B2M).

534. The use of any one of the preceding Items, wherein engineered cells (e.g., engineered CAR-T cells) of the first, second, and/or third population comprise reduced expression of a functional major histocompatibility complex class 11 human leukocyte antigen (HLA-II) complex relative to an unaltered or unmodified wild-type or control cell.

535. The use of any one of the preceding Items, wherein engineered cells (e.g., engineered CAR-T cells) of the first, second, and/or third population comprise one or more genetic modifications that reduce expression of one or more HLA-II molecules or HLA II associated molecules relative to an unaltered or unmodified wild-type or control cell.

536. The use of any one of the preceding Items, wherein engineered cells (e.g., engineered CAR-T cells) of the first, second, and/or third population do not express one or more HLA-II molecules or HLA II associated molecules.

537. The use of any one of the preceding Items, wherein the one or more HLA-II molecules comprise HLA-DP, HLA-DM, HLA-DOB, HLA-DQ, HLA-DR, or a combination thereof.

538. The use of any one of the preceding Items, wherein the one or more HLA-II molecules comprise HLA-DP.

539. The use of any one of the preceding Items, wherein the one or more HLA-II molecules comprise HLA-DM.

540. The use of any one of the preceding Items, wherein the one or more HLA-II molecules comprise HLA-DOB.

541. The use of any one of the preceding Items, wherein the one or more HLA-II molecules comprise HLA-DQ.

542. The use of any one of the preceding Items, wherein the one or more HLA-II molecules comprise HLA-DR.

543. The use of any one of the preceding Items, wherein the one or more HLA-II associated molecules comprise MHC class II transactivator (CIITA).

544. The use of any one of the preceding Items, wherein engineered cells (e.g., engineered CAR-T cells) of the first, second, and/or third population comprise reduced expression of RHD, ABO, PCDH11Y, NLGN4Y, or a combination thereof relative to an unaltered or unmodified wild-type or control cell.

545. The use of any one of the preceding Items, wherein engineered cells (e.g., engineered CAR-T cells) of the first, second, and/or third population comprise one or more genetic modifications that reduce expression of RHD, ABO, PCDH11Y, NLGN4Y, or a combination thereof relative to an unaltered or unmodified wild-type or control cell.

546. The use of any one of the preceding Items, wherein engineered cells (e.g., engineered CAR-T cells) of the first, second, and/or third population do not express RHD, ABO, PCDH11Y, NLGN4Y, or a combination thereof.

547. The use of any one of the preceding Items, wherein engineered cells (e.g., engineered CAR-T cells) of the first, second, and/or third population comprise reduced expression of a T cell receptor (TCR) relative to an unaltered or unmodified wild-type or control cell.

548. The use of any one of the preceding Items, wherein engineered cells (e.g., engineered CAR-T cells) of the first, second, and/or third population comprise one or more genetic modifications that reduce expression of a T cell receptor (TCR) relative to an unaltered or unmodified wild-type or control cell.

549. The use of any one of the preceding Items, wherein the TCR is a TCR-alpha (TRAC) and/or a TCR-beta (TRBC).

550. The use of any one of the preceding Items, wherein the first engineered cell (e.g., first engineered CAR-T cell) and/or the second engineered cell (e.g., second engineered CAR-T cell) do not express TRAC and/or TRBC.

551. The use of any one of the preceding Items, wherein the TCR is a TRAC.

552. The use of any one of the preceding Items, wherein the TCR is a TRBC.

553. The use of any one of the preceding Items, wherein engineered cells (e.g., engineered CAR-T cells) of the first, second, and/or third population comprise reduced expression of CD52 and/or CD70 relative to an unaltered or unmodified wild-type or control cell.

554. The use of any one of the preceding Items, wherein engineered cells (e.g., engineered CAR-T cells) of the first, second, and/or third population comprise one or more genetic modifications that reduce expression of CD52 and/or CD70 relative to an unaltered or unmodified wild-type or control cell.

555. The use of any one of the preceding Items, wherein engineered cells (e.g., engineered CAR-T cells) of the first, second, and/or third population do not express CD52 and/or CD70.

556. The use of any one of the preceding Items, wherein engineered cells (e.g., engineered CAR-T cells) of the first, second, and/or third population comprise reduced expression of PD-1 relative to an unaltered or unmodified wild-type or control cell.

557. The use of any one of the preceding Items, wherein engineered cells (e.g., engineered CAR-T cells) of the first, second, and/or third population comprise one or more genetic modifications that reduce expression of PD-1 relative to an unaltered or unmodified wild-type or control cell.

558. The use of any one of the preceding Items, wherein engineered cells (e.g., engineered CAR-T cells) of the first, second, and/or third population do not express PD-1.

559. The use of any one of the preceding Items, wherein the one or more genetic modifications comprise one or more gene knock downs.

560. The use of any one of the preceding Items, wherein the one or more genetic modifications are introduced by RNA silencing or RNA interference (RNAi).

561. The use of any one of the preceding Items, wherein RNA silencing or RNA interference (RNAi) comprising contacting a parental cell of the first engineered cell with short interfering RNAs (siRNAs), PIWI-interacting RNAs (piRNAs), short hairpin RNAs (shRNAs), and microRNAs (miRNAs).

562. The use of any one of the preceding Items, wherein the one or more genetic modifications are introduced by inducing an insertion or a deletion in the gene using a gene editing system.

563. The use of any one of the preceding Items, wherein engineered cells (e.g., engineered CAR-T cells) of the first, second, and/or third population comprise a genome editing system.

564. The use of any one of the preceding Items, wherein the gene editing system comprises a zinc finger nuclease (ZFN), a transcription activator-like effector nuclease (TALENs), a meganuclease, a transposase, a clustered regularly interspaced short palindromic repeat (CRISPR)/Cas system, a nickase system, a base editing system, a prime editing system, and/or a gene writing system.

565. The use of any one of the preceding Items, wherein the genome editing system comprises a genome targeting entity and a genome modifying entity.

566. The use of any one of the preceding Items, wherein the genome targeting entity comprises a nucleic acid-guided targeting entity.

567. The use of any one of the preceding Items, wherein the genome targeting entity comprises a sequence specific nuclease, a nucleic acid programmable DNA binding protein, an RNA guided nuclease, RNA-guided nuclease comprising a Cas nuclease and a guide RNA (CRISPR-Cas combination), a ribonucleoprotein (RNP) complex comprising a gRNA and a Cas nuclease, a homing endonuclease, a zinc finger nuclease (ZF) nucleic acid binding entity, a transcription activator-like effector (TALE) nucleic acid binding entity, a meganuclease, a Cas nuclease, a core Cas protein, a homing endonuclease, an endonuclease-deficient-Cas protein, an enzymatically inactive Cas protein, a CRISPR-associated transposase (CAST), a Type II or Type V Cas protein, or a functional portion thereof.

568. The use of any one of the preceding Items, wherein the genome targeting entity comprises Cas1, Cas2, Cas3, Cas4, Cas5, Cas6, Cas7, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr1, Cmr2, Cmr3, Cmr4, Cmr5, Cmr6, Csd1, Csd2, Cas5d, Cse1, Cse2, Cse3, Cse4, Cas5e, Csf1, Csm1, Csm2, Csm3, Csm4, Csm5, Csn1, Csn2, Cst1, Cst2, Cas5t, Csh1, Csh2, Cas5h, Csa1, Csa2, Csa3, Csa4, Csa5, Cas5a, Csx10, Csx11, Csy1, Csy2, Csy3, Csy4, Mad7, SpCas9, eSpCas9, SpCas9-HF1, HypaSpCas9, HeFSpCas9, and evoSpCas9 high-fidelity variants of SpCas9, SaCas9, NmeCas9, CjCas9, StCas9, TdCas9, LbCas12a, AsCas12a, AacCas12b, BhCas12b v4, TnpB, dCas (D10A), dCas (H840A), dCas13a, dCas13b, or a functional portion thereof.

569. The use of any one of the preceding Items, wherein the genome modifying entity cleaves, deaminates, nicks, polymerizes, interrogates, integrates, cuts, unwinds, breaks, alters, methylates, demethylates, or otherwise destabilizes the target locus.

570. The use of any one of the preceding Items, wherein the genome modifying entity comprises a recombinase, integrase, transposase, endonuclease, exonuclease, nickase, helicase, DNA polymerase, RNA polymerase, reverse transcriptase, deaminase, flippase, methylase, demethylase, acetylase, a nucleic acid modifying protein, an RNA modifying protein, a DNA modifying protein, an Argonaute protein, an epigenetic modifying protein, a histone modifying protein, or a functional portion thereof.

571. The use of any one of the preceding Items, wherein the genome modifying entity comprises a sequence specific nuclease, a nucleic acid programmable DNA binding protein, an RNA guided nuclease, RNA-guided nuclease comprising a Cas nuclease and a guide RNA (CRISPR-Cas combination), a ribonucleoprotein (RNP) complex comprising the gRNA and the Cas nuclease, a homing endonuclease, a zinc finger nuclease (ZFN), a transcription activator-like effector nuclease (TALEN), a meganuclease, a Cas nuclease, a core Cas protein, a TnpB nuclease, an endonuclease-deficient-Cas protein, an enzymatically inactive Cas protein, a CRISPR-associated transposase (CAST), a Type II or Type V Cas protein, base editing, prime editing, a Programmable Addition via Site-specific Targeting Elements (PASTE), or a functional portion thereof.

572. The use of any one of the preceding Items, wherein the genome modifying entity comprises Cas1, Cas2, Cas3, Cas4, Cas5, Cas6, Cas7, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr1, Cmr2, Cmr3, Cmr4, Cmr5, Cmr6, Csd1, Csd2, Cas5d, Cse1, Cse2, Cse3, Cse4, Cas5e, Csf1, Csm1, Csm2, Csm3, Csm4, Csm5, Csn1, Csn2, Cst1, Cst2, Cas5t, Csh1, Csh2, Cas5h, Csa1, Csa2, Csa3, Csa4, Csa5, Cas5a, Csx10, Csx11, Csy1, Csy2, Csy3, Csy4, Mad7, SpCas9, eSpCas9, SpCas9-HF1, HypaSpCas9, HeFSpCas9, and evoSpCas9 high-fidelity variants of SpCas9, SaCas9, NmeCas9, CjCas9, StCas9, TdCas9, LbCas12a, AsCas12a, AacCas12b, BhCas12b v4, TnpB, Fokl, dCas (D10A), dCas (H840A), dCas13a, dCas13b, a base editor, a prime editor, a target-primed reverse transcription (TPRT) editor, APOBECI, cytidine deaminase, adenosine deaminase, uracil glycosylase inhibitor (UGI), adenine base editors (ABE), cytosine base editors (CBE), reverse transcriptase, serine integrase, recombinase, transposase, polymerase, adenine-to-thymine or “ATBE” (or thymine-to-adenine or “TABE”) transversion base editor, ten-eleven translocation methylcytosine dioxygenases (TETs), TET1, TET3, TET1CD, histone acetyltransferase p300, histone methyltransferase SMYD3, histone methyltransferase PRDM9, H3K79 methyltransferase DOT1L, transcriptional repressor, or a functional portion thereof.

573. The use of any one of the preceding Items, wherein the genome targeting entity and the genome modifying entity are different domains of a single polypeptide.

574. The use of any one of the preceding Items, wherein the genome targeting entity and genome modifying entity are two different polypeptides that are operably linked together.

575. The use of any one of the preceding Items, wherein the genome targeting entity and genome modifying entity are two different polypeptides that are not linked together.

576. The use of any one of the preceding Items, wherein the genome editing complex comprises a guide nucleic acid having a targeting domain that is complementary to at least one sequence within the genomic safe harbor site, optionally wherein the guide nucleic acid is a guide RNA (gRNA).

577. The use of any one of the preceding Items, wherein the genome editing complex is an RNA-guided nuclease.

578. The use of any one of the preceding Items, wherein the RNA-guided nuclease comprises a Cas nuclease and a guide RNA (CRISPR-Cas combination).

579. The use of any one of the preceding Items, wherein the CRISPR-Cas combination is a ribonucleoprotein (RNP) complex comprising the gRNA and the Cas nuclease.

580. The use of any one of the preceding Items, wherein the Cas nuclease is a Type II or Type V Cas protein.

581. The use of any one of the preceding Items, wherein the Cas nuclease is Cas3, Cas4, Cas5, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr5, Cse1, Cse2, Csf1, Csm2, Csn2, Csx10, Csx11, Csy1, Csy2, Csy3, or Mad7.

582. The use of any one of the preceding Items, wherein the one or more genetic modifications are made at a modification site.

583. The use of any one of the preceding Items, wherein the modification site is 25 nucleotides or less from a protospacer adjacent motif (PAM) sequence, wherein the PAM sequence is ngg, nag, ngrrt, ngrrn, nnnngatt, nnnnryac, nnagaaw, naaaac, tttv, ttn, attn, tttn, gttn, or yttn and wherein: (ix) r=a or g, (x) y=c or t, (xi) w=a or t, (xii) v=a or c or g, and(xiii) n=a, c, t, or g.

584. The use of any one of the preceding Items, wherein the one or more genetic modifications are introduced using homology-directed repair (HDR)-mediated modification using SpCas9 and the PAM is ngg or nag, wherein n=a, c, t, or g.

585. The use of any one of the preceding Items, wherein the one or more genetic modifications are introduced using homology-directed repair (HDR)-mediated modification using SaCas9 and the PAM is ngrrt or ngrrn, wherein: (xiv) r=a or g, and (xv) n=a, c, t, or g.

586. The use of any one of the preceding Items, wherein the one or more genetic modifications are introduced using homology-directed repair (HDR)-mediated modification using NmeCas9 and the PAM is nnnngatt, wherein n=a, c, t, or g.

587. The use of any one of the preceding Items, wherein the one or more genetic modifications are introduced using homology-directed repair (HDR)-mediated modification using CjCas9 and the PAM is nnnnryac, wherein: (xvi) r=a or g, (xvii) y=c or t, and (xviii) n=a, c, t, or g.

588. The use of any one of the preceding Items, wherein the one or more genetic modifications are introduced using homology-directed repair (HDR)-mediated modification using StCas9 and the PAM is nnagaaw wherein: (xix) w=a or t, and (xx)n=a, c, t, or g.

589. The use of any one of the preceding Items, wherein the one or more genetic modifications are introduced using homology-directed repair (HDR)-mediated modification using TdCas9 and the PAM is naaaac, wherein n=a, c, t, or g.

590. The use of any one of the preceding Items, wherein the one or more genetic modifications are introduced using homology-directed repair (HDR)-mediated modification using LbCas12a and the PAM is tttv, wherein v=a or c or g.

591. The use of any one of the preceding Items, wherein the one or more genetic modifications are introduced using homology-directed repair (HDR)-mediated modification using AsCas12a and the PAM is tttv, wherein v=a or c or g.

592. The use of any one of the preceding Items, wherein the one or more genetic modifications are introduced using homology-directed repair (HDR)-mediated modification using AacCas12b and the PAM is ttn, wherein n=a, c, t, or g.

593. The use of any one of the preceding Items, wherein the one or more genetic modifications are introduced using homology-directed repair (HDR)-mediated modification using BhCas12b and the PAM is attn., tttn, or gttn, wherein n=a, c, t, or g.

594. The use of any one of the preceding Items, wherein the one or more genetic modifications are introduced using homology-directed repair (HDR)-mediated modification using MAD7 (ErCasl2a) and the PAM is yttn, wherein: (xxi) y=c or t, and (xxii)n=a, c, t, or g.

595. The use of any one of the preceding Items, the one or more genetic modifications are introduced by inducing an insertion or a deletion in the gene using a gene editing system ex vivo from a donor subject.

596. The use of any one of the preceding Items, wherein engineered cells (e.g., engineered CAR-T cells) of the first, second, and/or third population comprise one or more exogenous polynucleotides that encode one or more tolerogenic factors.

597. The use of any one of the preceding Items, wherein the one or more tolerogenic factors comprise A20/TNFAIP3, C1-Inhibitor, CCL21, CCL22, CD16, CD16 Fc receptor, CD24, CD27, CD35, CD39, CD46, CD47, CD52, CD55, CD59, CD64, CD200, CR1, CTLA4-Ig, DUX4, FasL, H2-M3, HLA-C, HLA-E, HLA-E heavy chain, HLA-F, HLA-G, IDO1, IL-10, IL15-RF, IL-35, IL-39, MANF, Mfge8, PD-L1, Serpinb9, CCL21, CCL22, B2M-HLA-E, C1 inhibitor, CR1, or a combination thereof.

598. The use of any one of the preceding Items, wherein engineered cells (e.g., engineered CAR-T cells) of the first, second, and/or third population comprise an exogenous polynucleotides that encode CD24.

599. The use of any one of the preceding Items, wherein engineered cells (e.g., engineered CAR-T cells) of the first, second, and/or third population comprise an exogenous polynucleotides that encode CD47.

600. The use of any one of the preceding Items, wherein engineered cells (e.g., engineered CAR-T cells) of the first, second, and/or third population comprise an exogenous polynucleotides that encode CD52.

601. The use of any one of the preceding Items, wherein engineered cells (e.g., engineered CAR-T cells) of the first, second, and/or third population comprise an exogenous polynucleotides that encode CD70.

602. The use of any one of the preceding Items, wherein engineered cells (e.g., engineered CAR-T cells) of the first, second, and/or third population express A20/TNFAIP3, C1-Inhibitor, CCL21, CCL22, CD16, CD16 Fc receptor, CD24, CD27, CD35, CD39, CD46, CD47, CD52, CD55, CD59, CD64, CD200, CR1, CTLA4-Ig, DUX4, FasL, H2-M3, HLA-C, HLA-E, HLA-E heavy chain, HLA-F, HLA-G, IDO1, IL-10, IL15-RF, IL-35, IL-39, MANF, Mfge8, PD-L1, Serpinb9, CCL21, CCL22, B2M-HLA-E, C1 inhibitor, CR1, and any combination thereof from one or more exogenous polynucleotides.

603. The use of any one of the preceding Items, wherein engineered cells (e.g., engineered CAR-T cells) of the first, second, and/or third population express CD47, HLA-E, and PD-L1 from one or more exogenous polynucleotides.

604. The use of any one of the preceding Items, wherein engineered cells (e.g., engineered CAR-T cells) of the first, second, and/or third population express CD24 from an exogenous polynucleotide.

605. The use of any one of the preceding Items, wherein engineered cells (e.g., engineered CAR-T cells) of the first, second, and/or third population express CD47 from an exogenous polynucleotide.

606. The use of any one of the preceding Items, wherein engineered cells (e.g., engineered CAR-T cells) of the first, second, and/or third population express CD52 from an exogenous polynucleotide.

607. The use of any one of the preceding Items, wherein engineered cells (e.g., engineered CAR-T cells) of the first, second, and/or third population express CD70 from an exogenous polynucleotide.

608. The use of any one of the preceding Items, wherein engineered cells (e.g., engineered CAR-T cells) of the first, second, and/or third population express CD47 from one or more exogenous polynucleotides.

609. The use of any one of the preceding Items, wherein one or more exogenous polynucleotides encoding one or more tolerogenic factors and/or one or more exogenous polynucleotides encoding one or more CARs are introduced at a safe harbor locus, a target locus, an RHD locus, a B2M locus, a CIITA locus, a TRAC locus, or a TRB locus.

610. The use of any one of the preceding Items, wherein the safe harbor locus is a CCR5 locus, a PPP1R12C locus, a CLYBL locus, or a Rosa locus.

611. The use of any one of the preceding Items, wherein the target locus is a CXCR4 locus, an ALB locus, a SHS231 locus, an F3 (CD142) locus, a MICA locus, a MICB locus, a LRP1 (CD91) locus, a HMGB1 locus, an ABO locus, a FUT1 locus, or a KDM5D locus.

612. The use of any one of the preceding Items, wherein one or more exogenous polynucleotides encoding one or more tolerogenic factors and/or one or more exogenous polynucleotides encoding one or more CARs are introduced into the first engineered cell using a gene therapy vector or a transposase system.

613. The use of any one of the preceding Items, wherein the transposase system comprises a transposase, a PiggyBac transposon, a Sleeping Beauty (SB11) transposon, a Mos1 transposon, or a Tol2 transposon.

614. The use of any one of the preceding Items, wherein the gene therapy vector is a retrovirus or a fusosome.

615. The use of any one of the preceding Items, wherein one or more exogenous polynucleotides encoding one or more tolerogenic factors and/or one or more exogenous polynucleotides encoding one or more CARs are encoded by a polycistronic vector.

616. The use of any one of the preceding Items, wherein the polycistronic vector is a bicistronic vector comprising one exogenous polynucleotide encoding a tolerogenic factor and one exogenous polynucleotide encoding one or more CARs.

617. The use of any one of the preceding Items, wherein engineered cells (e.g., engineered CAR-T cells) of the first, second, and/or third population comprise reduced expression of a HLA-I complex or reduced expression of a HLA-II complex relative to an unaltered or unmodified wild-type or control cell.

618. The use of any one of the preceding Items, wherein engineered cells (e.g., engineered CAR-T cells) of the first, second, and/or third population comprise reduced expression of B2M or reduced expression of CIITA relative to an unaltered or unmodified wild-type or control cell.

619. The use of any one of the preceding Items, wherein engineered cells (e.g., engineered CAR-T cells) of the first, second, and/or third population comprise (i) reduced expression of a HLA-I complex and (ii) reduced expression of a HLA-II complex relative to an unaltered or unmodified wild-type or control cell.

620. The use of any one of the preceding Items, wherein engineered cells (e.g., engineered CAR-T cells) of the first, second, and/or third population comprise (i) reduced expression of B2M and (ii) reduced expression of CIITA relative to an unaltered or unmodified wild-type or control cell.

621. The use of any one of the preceding Items, wherein engineered cells (e.g., engineered CAR-T cells) of the first, second, and/or third population comprise (i) reduced expression of a HLA-I complex or reduced expression of a HLA-II complex relative to an unaltered or unmodified wild-type or control cell, and (ii) increased expression of CD47 relative to an unaltered or unmodified wild-type or control cell.

622. The use of any one of the preceding Items, wherein engineered cells (e.g., engineered CAR-T cells) of the first, second, and/or third population comprise (i) reduced expression of B2M or reduced expression of CIITA relative to an unaltered or unmodified wild-type or control cell, and (ii) increased expression of CD47 relative to an unaltered or unmodified wild-type or control cell.

623. The use of any one of the preceding Items, wherein engineered cells (e.g., engineered CAR-T cells) of the first, second, and/or third population comprise (i) reduced expression of a HLA-I complex relative to an unaltered or unmodified wild-type or control cell, (ii) reduced expression of a HLA-II complex relative to an unaltered or unmodified wild-type or control cell, and (iii) increased expression of CD47 relative to an unaltered or unmodified wild-type or control cell.

624. The use of any one of the preceding Items, wherein engineered cells (e.g., engineered CAR-T cells) of the first, second, and/or third population comprise (i) reduced expression of B2M relative to an unaltered or unmodified wild-type or control cell, (ii) reduced expression of CIITA relative to an unaltered or unmodified wild-type or control cell, and (iii) increased expression of CD47 relative to an unaltered or unmodified wild-type or control cell.

625. The use of any one of the preceding Items, wherein engineered cells (e.g., engineered CAR-T cells) of the first, second, and/or third population comprise (i) reduced expression of a HLA-I complex or reduced expression of a HLA-II complex, and (ii) reduced expression of a TCR relative to an unaltered or unmodified wild-type or control cell.

626. The use of any one of the preceding Items, wherein engineered cells (e.g., engineered CAR-T cells) of the first, second, and/or third population comprise (i) reduced expression of B2M or reduced expression of CIITA, and (ii) reduced expression of a TRAC relative to an unaltered or unmodified wild-type or control cell.

627. The use of any one of the preceding Items, wherein engineered cells (e.g., engineered CAR-T cells) of the first, second, and/or third population comprise (i) reduced expression of a HLA-I complex, (ii) reduced expression of a HLA-II complex, and (iii) reduced expression of a TCR relative to an unaltered or unmodified wild-type or control cell.

628. The use of any one of the preceding Items, wherein engineered cells (e.g., engineered CAR-T cells) of the first, second, and/or third population comprise (i) reduced expression of B2M, (ii) reduced expression of CIITA, and (iii) reduced expression of a TRAC relative to an unaltered or unmodified wild-type or control cell.

629. The use of any one of the preceding Items, wherein engineered cells (e.g., engineered CAR-T cells) of the first, second, and/or third population comprise (i) reduced expression of a HLA-I complex or reduced expression of a HLA-II complex relative to an unaltered or unmodified wild-type or control cell, (ii) reduced expression of a TRAC relative to an unaltered or unmodified wild-type or control cell, and (iii) increased expression of CD47 relative to an unaltered or unmodified wild-type or control cell.

630. The use of any one of the preceding Items, wherein engineered cells (e.g., engineered CAR-T cells) of the first, second, and/or third population comprise (i) reduced expression of B2M or reduced expression of CIITA relative to an unaltered or unmodified wild-type or control cell, (ii) reduced expression of a TRAC relative to an unaltered or unmodified wild-type or control cell, and (ii) increased expression of CD47 relative to an unaltered or unmodified wild-type or control cell.

631. The use of any one of the preceding Items, wherein engineered cells (e.g., engineered CAR-T cells) of the first, second, and/or third population comprise (i) reduced expression of a HLA-I complex relative to an unaltered or unmodified wild-type or control cell, (ii) reduced expression of a HLA-II complex relative to an unaltered or unmodified wild-type or control cell, (iii) reduced expression of a TRAC relative to an unaltered or unmodified wild-type or control cell, and (iv) increased expression of CD47 relative to an unaltered or unmodified wild-type or control cell.

632. The use of any one of the preceding Items, wherein engineered cells (e.g., engineered CAR-T cells) of the first, second, and/or third population comprise (i) reduced expression of B2M relative to an unaltered or unmodified wild-type or control cell, (ii) reduced expression of CIITA relative to an unaltered or unmodified wild-type or control cell, (iii) reduced expression of a TRAC relative to an unaltered or unmodified wild-type or control cell, and (iv) increased expression of CD47 relative to an unaltered or unmodified wild-type or control cell.

633. The use of any one of the preceding Items, wherein engineered cells (e.g., engineered CAR-T cells) of the first, second, and/or third population comprise (i) reduced expression of B2M or reduced expression of CD52, and (ii) reduced expression of a TRAC relative to an unaltered or unmodified wild-type or control cell.

634. The use of any one of the preceding Items, wherein engineered cells (e.g., engineered CAR-T cells) of the first, second, and/or third population comprise (i) reduced expression of B2M, (ii) reduced expression of CD52, and (iii) reduced expression of a TRAC relative to an unaltered or unmodified wild-type or control cell.

635. The use of any one of the preceding Items, wherein engineered cells (e.g., engineered CAR-T cells) of the first, second, and/or third population comprise (i) reduced expression of B2M or reduced expression of CD70, and (ii) reduced expression of a TRAC relative to an unaltered or unmodified wild-type or control cell.

636. The use of any one of the preceding Items, wherein engineered cells (e.g., engineered CAR-T cells) of the first, second, and/or third population comprise (i) reduced expression of B2M, (ii) reduced expression of CD70, and (iii) reduced expression of a TRAC relative to an unaltered or unmodified wild-type or control cell.

637. The use of any one of the preceding Items, wherein engineered cells (e.g., engineered CAR-T cells) of the first, second, and/or third population comprise (i) reduced expression of PD-1, and (ii) reduced expression of a TRAC relative to an unaltered or unmodified wild-type or control cell.

638. The use of any one of the preceding Items, wherein engineered cells (e.g., engineered CAR-T cells) of the first, second, and/or third population comprise B2M^{indel/indel}, CIITA^{indel/indel}, TRAC^{indel/indel}, and/or TRAC^{indel/indel} cells.

639. The use of any one of the preceding Items, wherein the disease or disorder is characterized by antigen evasion or antigenic drift, and wherein the one or more targeted therapies were administered to the patient prior to antigen evasion or antigenic drift.

640. The use of any one of the preceding Items, wherein the disease or disorder is characterized by antigen evasion or antigenic drift, and wherein the therapeutic agent is administered to the patient after antigen evasion or antigenic drift.

641. The use of any one of the preceding Items, wherein the patient is at risk of antigen evasion, and wherein the therapeutic agent is administered to the patient before antigen evasion.

642. The use of any one of the preceding Items, wherein the patient is at risk of antigen evasion, and wherein the therapeutic agent is administered to the patient before antigenic drift.

643. The use of any one of the preceding Items, wherein the patient has been diagnosed with the disease or disorder.

644. The use of any one of the preceding Items, wherein the therapeutic agent comprises a first population of the engineered cells, and wherein the first population of engineered cells evade NK cell mediated cytotoxicity upon administration to the recipient patient.

645. The use of any one of the preceding Items, wherein the therapeutic agent comprises a first population of the engineered cells, and wherein the first population of engineered cells are protected from cell lysis by mature NK cells upon administration to the recipient patient.

646. The use of any one of the preceding Items, wherein the therapeutic agent comprises a first population of the engineered cells, and wherein the first population of engineered cells evade macrophage-mediated cytotoxicity, optionally wherein the macrophage-mediated cytotoxicity involves phagocytosis and/or reactive oxygen species.

647. The use of any one of the preceding Items, wherein the therapeutic agent comprises a first population of the engineered cells, and wherein the first population of engineered cells do not induce an immune response to the cell upon administration to the recipient patient.

648. The use of any one of the preceding Items, wherein the therapeutic agent comprises a first population of the engineered cells, and wherein the first population of engineered cells persist in the patient for at least 4 weeks, at least 2 months, at least 3 months, at least 4 months, at least 5 months, at least 6 months, at least 7 months, at least 8 months, at least 9 months, at least 10 months, at least 11 months, at least 12 months, or longer.

649. The use of any one of the preceding Items, wherein the therapeutic agent comprises a first population of the engineered cells, and wherein the first population of engineered cells comprise an autologous or allogeneic cell-based therapy, and wherein the population of first engineered cells persist in the patient for longer than cells of the one or more targeted therapies.

650. The use of any one of the preceding Items, wherein the therapeutic agent comprises a first population of the engineered cells, and wherein the first population of engineered cells lasts for a duration of at least 4 weeks, at least 2 months, at least 3 months, at least 4 months, at least 5 months, at least 6 months, at least 7 months, at least 8 months, at least 9 months, at least 10 months, at least 11 months, at least 12 months, or longer.

651. The use of any one of the preceding Items, wherein the therapeutic effect of the first population of engineered cells lasts for longer than that of the one or more targeted therapies.

652. A population of engineered cells, wherein the engineered cells of the population comprise one or more CARs directed to a first therapeutic target and one or more CARs directed to a second therapeutic target.

653. The population of engineered cells according to any proceeding Item, wherein a first subset of the engineered cells of the population comprise one or more CARs directed to the first therapeutic target and wherein a second subset of the engineered cells of the population comprise one or more CARs directed to the second therapeutic target.

654. The population of engineered cells according to any proceeding Item, wherein a first subset of the engineered cells of the population comprise one or more CARs directed to the first therapeutic target, wherein a second subset of the engineered cells of the population comprise one or more CARs directed to the second therapeutic target, and wherein a third subset of the engineered cells of the population comprise one or more CARs directed to the first therapeutic target and one or more CARs directed to the second therapeutic target.

655. The population of engineered cells according to any proceeding Item, wherein the first therapeutic target is a first antigen.

656. The population of engineered cells according to any proceeding Item, wherein the first antigen is an antigen present on the surface of a B cell.

657. The population of engineered cells according to any proceeding Item, wherein the B cell is a malignant B cell.

658. The population of engineered cells according to any proceeding Item, wherein the first antigen is CD22, CD20, CD19, BCMA, GPRC5D, CD38, CD70, CD79b, HER2, IL13Ra2, MUC1, or a variant thereof.

659. The population of engineered cells according to any proceeding Item, wherein the first antigen is CD22, CD20, CD19, BCMA, GPRC5D, CD38, CD70, CD79b, HER2, IL13Ra2, or MUC1.

660. The population of engineered cells according to any proceeding Item, wherein the second therapeutic target is a second antigen.

661. The population of engineered cells according to any proceeding Item, wherein the second antigen is an antigen present on the surface of a B cell.

662. The population of engineered cells according to any proceeding Item, wherein the B cell is a malignant B cell.

663. The population of engineered cells according to any proceeding Item, wherein the second antigen is CD22, CD20, CD19, BCMA, GPRC5D, CD38, CD70, CD79b, HER2, IL13Ra2, MUC1, or a variant thereof.

664. The population of engineered cells according to any proceeding Item, wherein the second antigen is CD22, CD20, CD19, BCMA, GPRC5D, CD38, CD70, CD79b, HER2, IL13Ra2, or MUC1.

665. The population of engineered cells according to any proceeding Item, wherein the second antigen is CD22, CD20, or CD19.

666. The population of engineered cells according to any proceeding Item, wherein the first antigen binding domain is capable of binding to CD22 or a variant thereof.

667. The population of engineered cells according to any proceeding Item, wherein the first antigen binding domain is capable of binding to CD22.

668. The population of engineered cells according to any proceeding Item, wherein the first antigen binding domain comprises a heavy chain complementarity determining region 1 (HCDR1) comprising an amino acid sequence according to SEQ ID NO: 47, a heavy chain complementarity determining region 2 (HCDR2) comprising an amino acid sequence according to SEQ ID NO: 48, and a heavy chain complementarity determining region 3 (HCDR3) comprising an amino acid sequence according to SEQ ID NO: 49.

669. The population of engineered cells according to any proceeding Item, wherein the first antigen binding domain comprises a light chain complementarity determining region 1 (LCDR1) comprising an amino acid sequence according to SEQ ID NO: 51, a light chain complementarity determining region 2 (LCDR2) comprising an amino acid sequence according to SEQ ID NO: 52, and a light chain complementarity determining region 3 (LCDR3) comprising an amino acid sequence according to SEQ ID NO: 53.

670. The population of engineered cells of any one of claims X, wherein the first antigen binding domain comprises a heavy chain variable domain (VH) comprising an amino acid sequence according to SEQ ID NO: 47, an amino acid sequence according to SEQ ID NO: 48, and an amino acid sequence according to SEQ ID NO:49 arranged non-contiguously from N-terminus to C-terminus.

671. The population of engineered cells according to any proceeding Item, wherein the first antigen binding domain comprises a heavy chain variable domain (VH) comprising an amino acid sequence that is at least 80% identical, at least 80%, at least 85%, at least 90%, at least 95%, at least 96%, at least 97%, at least 98%, at least 99%, or 100% identical to an amino acid sequence according to SEQ ID NO: 46.

672. The population of engineered cells of any one of claims X, wherein the first antigen binding domain comprises a light chain variable domain (VL) comprising an amino acid sequence according to SEQ ID NO: 51, an amino acid sequence according to SEQ ID NO: 52, and an amino acid sequence according to SEQ ID NO: 53 arranged non-contiguously from N-terminus to C-terminus.

673. The population of engineered cells according to any proceeding Item, wherein the first antigen binding domain comprises a light chain variable domain (VL) comprising an amino acid sequence that is at least 80% identical, at least 80%, at least 85%, at least 90%, at least 95%, at least 96%, at least 97%, at least 98%, at least 99%, or 100% identical to an amino acid sequence according to SEQ ID NO: 50.

674. The population of engineered cells according to any proceeding Item, wherein the first antigen binding domain comprises an HCDR1 according to SEQ ID NO: 56, an HCDR2 according to SEQ ID NO: 57, and an HCDR3 according to SEQ ID NO: 58.

675. The population of engineered cells according to any proceeding Item, wherein the first antigen binding domain comprises an LCDR1 according to SEQ ID NO: 60, an LCDR2 according to SEQ ID NO: 61, and an LCDR3 according to SEQ ID NO: 62.

676. The population of engineered cells of any one of claims X, wherein the first antigen binding domain comprises a heavy chain variable domain (VH) comprising an amino acid sequence according to SEQ ID NO: 56, an amino acid sequence according to SEQ ID NO: 57, and an amino acid sequence according to SEQ ID NO: 58 arranged non-contiguously from N-terminus to C-terminus.

677. The population of engineered cells according to any proceeding Item, wherein the first antigen binding domain comprises a heavy chain variable domain (VH) comprising an amino acid sequence that is at least 80% identical, at least 80%, at least 85%, at least 90%, at least 95%, at least 96%, at least 97%, at least 98%, at least 99%, or 100% identical to an amino acid sequence according to SEQ ID NO: 55.

678. The population of engineered cells of any one of claims X, wherein the first antigen binding domain comprises a light chain variable domain (VL) comprising an amino acid sequence according to SEQ ID NO: 60, an amino acid sequence according to SEQ ID NO: 61, and an amino acid sequence according to SEQ ID NO: 62 arranged non-contiguously from N-terminus to C-terminus.

679. The population of engineered cells according to any proceeding Item, wherein the first antigen binding domain comprises a light chain variable domain (VL) comprising an amino acid sequence that is at least 80% identical, at least 80%, at least 85%, at least 90%, at least 95%, at least 96%, at least 97%, at least 98%, at least 99%, or 100% identical to an amino acid sequence according to SEQ ID NO: 59.

680. The population of engineered cells according to any proceeding Item, wherein the first antigen binding domain is capable of binding to CD19 or a variant thereof.

681. The population of engineered cells according to any proceeding Item, wherein the first antigen binding domain is capable of binding to CD19.

682. The population of engineered cells according to any proceeding Item, wherein the first antigen binding domain comprises an HCDR1 according to SEQ ID NO: 26, an HCDR2 according to SEQ ID NO: 27, and an HCDR3 according to SEQ ID NO: 28.

683. The population of engineered cells according to any proceeding Item, wherein the first antigen binding domain comprises an LCDR1 according to SEQ ID NO: 21, an LCDR2 according to SEQ ID NO: 22, and an LCDR3 according to SEQ ID NO: 23.

684. The population of engineered cells of any one of claims X, wherein the first antigen binding domain comprises a heavy chain variable domain (VH) comprising an amino acid sequence according to SEQ ID NO: 26, an amino acid sequence according to SEQ ID NO: 27, and an amino acid sequence according to SEQ ID NO: 28 arranged non-contiguously from N-terminus to C-terminus.

685. The population of engineered cells according to any proceeding Item, wherein the first antigen binding domain comprises a heavy chain variable domain (VH) comprising an amino acid sequence that is at least 80% identical, at least 80%, at least 85%, at least 90%, at least 95%, at least 96%, at least 97%, at least 98%, at least 99%, or 100% identical to an amino acid sequence according to SEQ ID NO: 25.

686. The population of engineered cells of any one of claims X, wherein the first antigen binding domain comprises a light chain variable domain (VL) comprising an amino acid sequence according to SEQ ID NO: 21, an amino acid sequence according to SEQ ID NO: 22, and an amino acid sequence according to SEQ ID NO: 23 arranged non-contiguously from N-terminus to C-terminus.

687. The population of engineered cells according to any proceeding Item, wherein the first antigen binding domain comprises a light chain variable domain (VL) comprising an amino acid sequence that is at least 80% identical, at least 80%, at least 85%, at least 90%, at least 95%, at least 96%, at least 97%, at least 98%, at least 99%, or 100% identical to an amino acid sequence according to SEQ ID NO: 20.

688. The population of engineered cells according to any proceeding Item, wherein the first antigen binding domain is capable of binding to CD20 or a variant thereof.

689. The population of engineered cells according to any proceeding Item, wherein the first antigen binding domain is capable of binding to CD20.

690. The population of engineered cells according to any proceeding Item, wherein the first antigen binding domain comprises an HCDR1 according to SEQ ID NO: 43, an HCDR2 according to SEQ ID NO: 44, and an HCDR3 according to SEQ ID NO: 107.

691. The population of engineered cells according to any proceeding Item, wherein the first antigen binding domain comprises an LCDR1 according to SEQ ID NO: 39, an LCDR2 according to SEQ ID NO: 40, and an LCDR3 according to SEQ ID NO: 41.

692. The population of engineered cells of any one of claims X, wherein the first antigen binding domain comprises a heavy chain variable domain (VH) comprising an amino acid sequence according to SEQ ID NO: 43, an amino acid sequence according to SEQ ID NO: 44, and an amino acid sequence according to SEQ ID NO: 107 arranged non-contiguously from N-terminus to C-terminus.

693. The population of engineered cells according to any proceeding Item, wherein the first antigen binding domain comprises a heavy chain variable domain (VH) comprising an amino acid sequence that is at least 80% identical, at least 80%, at least 85%, at least 90%, at least 95%, at least 96%, at least 97%, at least 98%, at least 99%, or 100% identical to an amino acid sequence according to SEQ ID NO: 42.

694. The population of engineered cells of any one of claims X, wherein the first antigen binding domain comprises a light chain variable domain (VL) comprising an amino acid sequence according to SEQ ID NO: 39, an amino acid sequence according to SEQ ID NO: 40, and an amino acid sequence according to SEQ ID NO: 41 arranged non-contiguously from N-terminus to C-terminus.

695. The population of engineered cells according to any proceeding Item, wherein the first antigen binding domain comprises a light chain variable domain (VL) comprising an amino acid sequence that is at least 80% identical, at least 80%, at least 85%, at least 90%, at least 95%, at least 96%, at least 97%, at least 98%, at least 99%, or 100% identical to an amino acid sequence according to SEQ ID NO: 38.

696. The population of engineered cells according to any proceeding Item, wherein the second antigen is CD19 or a variant thereof.

697. The population of engineered cells according to any proceeding Item, wherein the second antigen is CD19.

698. The population of engineered cells according to any proceeding Item, wherein the second antigen binding domain comprises an HCDR1 according to SEQ ID NO: 26, an HCDR2 according to SEQ ID NO: 27, and an HCDR3 according to SEQ ID NO: 28.

699. The population of engineered cells according to any proceeding Item, wherein the second antigen binding domain comprises an LCDR1 according to SEQ ID NO: 21, an LCDR2 according to SEQ ID NO: 22, and an LCDR3 according to SEQ ID NO: 23.

700. The population of engineered cells of any one of claims X, wherein the first antigen binding domain comprises a heavy chain variable domain (VH) comprising an amino acid sequence according to SEQ ID NO: 26, an amino acid sequence according to SEQ ID NO: 27, and an amino acid sequence according to SEQ ID NO: 28 arranged non-contiguously from N-terminus to C-terminus.

701. The population of engineered cells according to any proceeding Item, wherein the second antigen binding domain comprises a heavy chain variable domain (VH) comprising an amino acid sequence that is at least 80% identical, at least 80%, at least 85%, at least 90%, at least 95%, at least 96%, at least 97%, at least 98%, at least 99%, or 100% identical to an amino acid sequence according to SEQ ID NO: 25.

702. The population of engineered cells of any one of claims X, wherein the first antigen binding domain comprises a light chain variable domain (VL) comprising an amino acid sequence according to SEQ ID NO: 21, an amino acid sequence according to SEQ ID NO: 22, and an amino acid sequence according to SEQ ID NO: 23 arranged non-contiguously from N-terminus to C-terminus.

703. The population of engineered cells according to any proceeding Item, wherein the second antigen binding domain comprises a light chain variable domain (VL) comprising an amino acid sequence that is at least 80% identical, at least 80%, at least 85%, at least 90%, at least 95%, at least 96%, at least 97%, at least 98%, at least 99%, or 100% identical to an amino acid sequence according to SEQ ID NO: 20.

704. The population of engineered cells according to any proceeding Item, wherein the second antigen is CD20 or a variant thereof.

705. The population of engineered cells according to any proceeding Item, wherein the second antigen is CD20.

706. The population of engineered cells according to any proceeding Item, wherein the second antigen binding domain comprises an HCDR1 according to SEQ ID NO: 43, an HCDR2 according to SEQ ID NO: 44, and an HCDR3 according to SEQ ID NO: 107.

707. The population of engineered cells according to any proceeding Item, wherein the second antigen binding domain comprises an LCDR1 according to SEQ ID NO: 39, an LCDR2 according to SEQ ID NO: 40, and an LCDR3 according to SEQ ID NO: 41.

708. The population of engineered cells of any one of claims X, wherein the first antigen binding domain comprises a heavy chain variable domain (VH) comprising an amino acid sequence according to SEQ ID NO: 43, an amino acid sequence according to SEQ ID NO: 44, and an amino acid sequence according to SEQ ID NO: 107 arranged non-contiguously from N-terminus to C-terminus.

709. The population of engineered cells according to any proceeding Item, wherein the second antigen binding domain comprises a heavy chain variable domain (VH) comprising an amino acid sequence that is at least 80% identical, at least 80%, at least 85%, at least 90%, at least 95%, at least 96%, at least 97%, at least 98%, at least 99%, or 100% identical to an amino acid sequence according to SEQ ID NO: 42.

710. The population of engineered cells of any one of claims X, wherein the first antigen binding domain comprises a light chain variable domain (VL) comprising an amino acid sequence according to SEQ ID NO: 39, an amino acid sequence according to SEQ ID NO: 40, and an amino acid sequence according to SEQ ID NO: 41 arranged non-contiguously from N-terminus to C-terminus.

711. The population of engineered cells according to any proceeding Item, wherein the second antigen binding domain comprises a light chain variable domain (VL) comprising an amino acid sequence that is at least 80% identical, at least 80%, at least 85%, at least 90%, at least 95%, at least 96%, at least 97%, at least 98%, at least 99%, or 100% identical to an amino acid sequence according to SEQ ID NO: 38.

712. The population of engineered cells according to any proceeding Item, wherein the second antigen is CD22 or a variant thereof.

713. The population of engineered cells according to any proceeding Item, wherein the second antigen is CD22.

714. The population of engineered cells according to any proceeding Item, wherein the second antigen binding domain comprises an HCDR1 according to SEQ ID NO: 47, an HCDR2 according to SEQ ID NO: 48, and an HCDR3 according to SEQ ID NO: 49.

715. The population of engineered cells according to any proceeding Item, wherein the second antigen binding domain comprises an LCDR1 according to SEQ ID NO: 51, an LCDR2 according to SEQ ID NO: 52, and an LCDR3 according to SEQ ID NO: 53.

716. The population of engineered cells of any one of claims X, wherein the first antigen binding domain comprises a heavy chain variable domain (VH) comprising an amino acid sequence according to SEQ ID NO: 47, an amino acid sequence according to SEQ ID NO: 48, and an amino acid sequence according to SEQ ID NO: 49 arranged non-contiguously from N-terminus to C-terminus.

717. The population of engineered cells according to any proceeding Item, wherein the second antigen binding domain comprises a heavy chain variable domain (VH) comprising an amino acid sequence that is at least 80% identical, at least 80%, at least 85%, at least 90%, at least 95%, at least 96%, at least 97%, at least 98%, at least 99%, or 100% identical to an amino acid sequence according to SEQ ID NO: 46.

718. The population of engineered cells of any one of claims X, wherein the first antigen binding domain comprises a light chain variable domain (VL) comprising an amino acid sequence according to SEQ ID NO: 51, an amino acid sequence according to SEQ ID NO: 52, and an amino acid sequence according to SEQ ID NO: 53 arranged non-contiguously from N-terminus to C-terminus.

719. The population of engineered cells according to any proceeding Item, wherein the second antigen binding domain comprises a light chain variable domain (VL) comprising an amino acid sequence that is at least 80% identical, at least 80%, at least 85%, at least 90%, at least 95%, at least 96%, at least 97%, at least 98%, at least 99%, or 100% identical to an amino acid sequence according to SEQ ID NO: 50.

720. The population of engineered cells according to any proceeding Item, wherein the engineered cells of the population comprise reduced expression of a functional major histocompatibility complex class I human leukocyte antigen (HLA-1) complex relative to an unaltered or unmodified wild-type or control cell.

721. The population of engineered cells according to any proceeding Item, wherein the engineered cells of the population comprise one or more genetic modifications that reduce expression of one or more HLA-I molecules or HLA I associated molecules relative to an unaltered or unmodified wild-type or control cell.

722. The population of engineered cells according to any proceeding Item, wherein the engineered cells of the population do not express one or more HLA-I molecules or HLA I associated molecules.

723. The population of engineered cells according to any proceeding Item, wherein the one or more HLA-I molecules comprise HLA-A, HLA-B, HLA-C, or a combination thereof.

724. The population of engineered cells according to any proceeding Item, wherein the one or more HLA-I molecules comprise HLA-A.

725. The population of engineered cells according to any proceeding Item, wherein the one or more HLA-I molecules comprise HLA-B.

726. The population of engineered cells according to any proceeding Item, wherein the one or more HLA-I molecules comprise HLA-C.

727. The population of engineered cells according to any proceeding Item, wherein the one or more HLA-I associated molecules comprise β-2 microglobulin (B2M).

728. The population of engineered cells according to any proceeding Item, wherein the engineered cells of the population comprise reduced expression of a functional major histocompatibility complex class II human leukocyte antigen (HLA-II) complex relative to an unaltered or unmodified wild-type or control cell.

729. The population of engineered cells according to any proceeding Item, wherein the engineered cells of the population comprise one or more genetic modifications that reduce expression of one or more HLA-II molecules or HLA 11 associated molecules relative to an unaltered or unmodified wild-type or control cell.

730. The population of engineered cells according to any proceeding Item, wherein the engineered cells of the population do not express one or more HLA-II molecules or HLA 11 associated molecules.

731. The population of engineered cells according to any proceeding Item, wherein the one or more HLA-II molecules comprise HLA-DP, HLA-DM, HLA-DOB, HLA-DQ, HLA-DR, or a combination thereof.

732. The population of engineered cells according to any proceeding Item, wherein the one or more HLA-II molecules comprise HLA-DP.

733. The population of engineered cells according to any proceeding Item, wherein the one or more HLA-II molecules comprise HLA-DM.

734. The population of engineered cells according to any proceeding Item, wherein the one or more HLA-II molecules comprise HLA-DOB.

735. The population of engineered cells according to any proceeding Item, wherein the one or more HLA-II molecules comprise HLA-DQ.

736. The population of engineered cells according to any proceeding Item, wherein the one or more HLA-II molecules comprise HLA-DR.

737. The population of engineered cells according to any proceeding Item, wherein the one or more HLA-II associated molecules comprise MHC class 11 transactivator (CIITA).

738. The population of engineered cells according to any proceeding Item, wherein the engineered cells of the population comprise reduced expression of RHD, ABO, PCDH11Y, NLGN4Y, or a combination thereof relative to an unaltered or unmodified wild-type or control cell.

739. The population of engineered cells according to any proceeding Item, wherein the engineered cells of the population comprise one or more genetic modifications that reduce expression of RHD, ABO, PCDH11Y, NLGN4Y, or a combination thereof relative to an unaltered or unmodified wild-type or control cell.

740. The population of engineered cells according to any proceeding Item, wherein the engineered cells of the population do not express RHD, ABO, PCDH11Y, NLGN4Y, or a combination thereof.

741. The population of engineered cells according to any proceeding Item, wherein the engineered cells of the population comprise reduced expression of a T cell receptor (TCR) relative to an unaltered or unmodified wild-type or control cell.

742. The population of engineered cells according to any proceeding Item, wherein the engineered cells of the population comprise one or more genetic modifications that reduce expression of a T cell receptor (TCR) relative to an unaltered or unmodified wild-type or control cell.

743. The population of engineered cells according to any proceeding Item, wherein the TCR is a TCR-alpha (TRAC) and/or a TCR-beta (TRBC).

744. The population of engineered cells according to any proceeding Item, wherein the engineered cells of the population do not express TRAC and/or TRBC.

745. The population of engineered cells according to any proceeding Item, wherein the TCR is a TRAC.

746. The population of engineered cells according to any proceeding Item, wherein the TCR is a TRBC.

747. The population of engineered cells according to any proceeding Item, wherein the engineered cells of the population comprise reduced expression of CD52 and/or CD70 relative to an unaltered or unmodified wild-type or control cell.

748. The population of engineered cells according to any proceeding Item, wherein the engineered cells of the population comprise one or more genetic modifications that reduce expression of CD52 and/or CD70 relative to an unaltered or unmodified wild-type or control cell.

749. The population of engineered cells according to any proceeding Item, wherein the engineered cells of the population do not express CD52 and/or CD70.

750. The population of engineered cells according to any proceeding Item, wherein the engineered cells of the population comprise reduced expression of PD-1 relative to an unaltered or unmodified wild-type or control cell.

751. The population of engineered cells according to any proceeding Item, wherein the engineered cells of the population comprise one or more genetic modifications that reduce expression of PD-1 relative to an unaltered or unmodified wild-type or control cell.

752. The population of engineered cells according to any proceeding Item, wherein the engineered cells of the population do not express PD-1.

753. The population of engineered cells according to any proceeding Item, wherein the one or more genetic modifications comprise one or more gene knock downs.

754. The population of engineered cells according to any proceeding Item, wherein the one or more genetic modifications are introduced by RNA silencing or RNA interference (RNAi).

755. The population of engineered cells according to any proceeding Item, wherein RNA silencing or RNA interference (RNAi) comprising contacting a parental cell of the first engineered cell with short interfering RNAs (siRNAs), PIWI-interacting RNAs (piRNAs), short hairpin RNAs (shRNAs), and microRNAs (miRNAs).

756. The population of engineered cells according to any proceeding Item, wherein the one or more genetic modifications are introduced by inducing an insertion or a deletion in the gene using a gene editing system.

757. The population of engineered cells according to any proceeding Item, wherein engineered cells (e.g., engineered CAR-T cells) of the first, second, and/or third population comprise a genome editing system.

758. The population of engineered cells according to any proceeding Item, wherein the gene editing system comprises a zinc finger nuclease (ZFN), a transcription activator-like effector nuclease (TALENs), a meganuclease, a transposase, a clustered regularly interspaced short palindromic repeat (CRISPR)/Cas system, a nickase system, a base editing system, a prime editing system, and/or a gene writing system.

759. The population of engineered cells according to any proceeding Item, wherein the genome editing system comprises a genome targeting entity and a genome modifying entity.

760. The population of engineered cells according to any proceeding Item, wherein the genome targeting entity comprises a nucleic acid-guided targeting entity.

761. The population of engineered cells according to any proceeding Item, wherein the genome targeting entity comprises a sequence specific nuclease, a nucleic acid programmable DNA binding protein, an RNA guided nuclease, RNA-guided nuclease comprising a Cas nuclease and a guide RNA (CRISPR-Cas combination), a ribonucleoprotein (RNP) complex comprising a gRNA and a Cas nuclease, a homing endonuclease, a zinc finger nuclease (ZF) nucleic acid binding entity, a transcription activator-like effector (TALE) nucleic acid binding entity, a meganuclease, a Cas nuclease, a core Cas protein, a homing endonuclease, an endonuclease-deficient-Cas protein, an enzymatically inactive Cas protein, a CRISPR-associated transposase (CAST), a Type II or Type V Cas protein, or a functional portion thereof.

762. The population of engineered cells according to any proceeding Item, wherein the genome targeting entity comprises Cas1, Cas2, Cas3, Cas4, Cas5, Cas6, Cas7, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr1, Cmr2, Cmr3, Cmr4, Cmr5, Cmr6, Csd1, Csd2, Cas5d, Cse1, Cse2, Cse3, Cse4, Cas5e, Csf1, Csm1, Csm2, Csm3, Csm4, Csm5, Csn1, Csn2, Cst1, Cst2, Cas5t, Csh1, Csh2, Cas5h, Csa1, Csa2, Csa3, Csa4, Csa5, Cas5a, Csx10, Csx11, Csy1, Csy2, Csy3, Csy4, Mad7, SpCas9, eSpCas9, SpCas9-HF1, HypaSpCas9, HeFSpCas9, and evoSpCas9 high-fidelity variants of SpCas9, SaCas9, NmeCas9, CjCas9, StCas9, TdCas9, LbCas12a, AsCas12a, AacCas12b, BhCas12b v4, TnpB, dCas (D10A), dCas (H840A), dCas13a, dCas13b, or a functional portion thereof.

763. The population of engineered cells according to any proceeding Item, wherein the genome modifying entity cleaves, deaminates, nicks, polymerizes, interrogates, integrates, cuts, unwinds, breaks, alters, methylates, demethylates, or otherwise destabilizes the target locus.

764. The population of engineered cells according to any proceeding Item, wherein the genome modifying entity comprises a recombinase, integrase, transposase, endonuclease, exonuclease, nickase, helicase, DNA polymerase, RNA polymerase, reverse transcriptase, deaminase, flippase, methylase, demethylase, acetylase, a nucleic acid modifying protein, an RNA modifying protein, a DNA modifying protein, an Argonaute protein, an epigenetic modifying protein, a histone modifying protein, or a functional portion thereof.

765. The population of engineered cells according to any proceeding Item, wherein the genome modifying entity comprises a sequence specific nuclease, a nucleic acid programmable DNA binding protein, an RNA guided nuclease, RNA-guided nuclease comprising a Cas nuclease and a guide RNA (CRISPR-Cas combination), a ribonucleoprotein (RNP) complex comprising the gRNA and the Cas nuclease, a homing endonuclease, a zinc finger nuclease (ZFN), a transcription activator-like effector nuclease (TALEN), a meganuclease, a Cas nuclease, a core Cas protein, a TnpB nuclease, an endonuclease-deficient-Cas protein, an enzymatically inactive Cas protein, a CRISPR-associated transposase (CAST), a Type II or Type V Cas protein, base editing, prime editing, a Programmable Addition via Site-specific Targeting Elements (PASTE), or a functional portion thereof.

766. The population of engineered cells according to any proceeding Item, wherein the genome modifying entity comprises Cas1, Cas2, Cas3, Cas4, Cas5, Cas6, Cas7, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr1, Cmr2, Cmr3, Cmr4, Cmr5, Cmr6, Csd1, Csd2, Cas5d, Cse1, Cse2, Cse3, Cse4, Cas5e, Csf1, Csm1, Csm2, Csm3, Csm4, Csm5, Csn1, Csn2, Cst1, Cst2, Cas5t, Csh1, Csh2, Cas5h, Csa1, Csa2, Csa3, Csa4, Csa5, Cas5a, Csx10, Csx11, Csy1, Csy2, Csy3, Csy4, Mad7, SpCas9, eSpCas9, SpCas9-HF1, HypaSpCas9, HeFSpCas9, and evoSpCas9 high-fidelity variants of SpCas9, SaCas9, NmeCas9, CjCas9, StCas9, TdCas9, LbCas12a, AsCas12a, AacCas12b, BhCas12b v4, TnpB, Fokl, dCas (D10A), dCas (H840A), dCas13a, dCas13b, a base editor, a prime editor, a target-primed reverse transcription (TPRT) editor, APOBECI, cytidine deaminase, adenosine deaminase, uracil glycosylase inhibitor (UGI), adenine base editors (ABE), cytosine base editors (CBE), reverse transcriptase, serine integrase, recombinase, transposase, polymerase, adenine-to-thymine or “ATBE” (or thymine-to-adenine or “TABE”) transversion base editor, ten-eleven translocation methylcytosine dioxygenases (TETs), TET1, TET3, TET1CD, histone acetyltransferase p300, histone methyltransferase SMYD3, histone methyltransferase PRDM9, H3K79 methyltransferase DOT1L, transcriptional repressor, or a functional portion thereof.

767. The population of engineered cells according to any proceeding Item, wherein the genome targeting entity and the genome modifying entity are different domains of a single polypeptide.

768. The population of engineered cells according to any proceeding Item, wherein the genome targeting entity and genome modifying entity are two different polypeptides that are operably linked together.

769. The population of engineered cells according to any proceeding Item, wherein the genome targeting entity and genome modifying entity are two different polypeptides that are not linked together.

770. The population of engineered cells according to any proceeding Item, wherein the genome editing complex comprises a guide nucleic acid having a targeting domain that is complementary to at least one sequence within the genomic safe harbor site, optionally wherein the guide nucleic acid is a guide RNA (gRNA).

771. The population of engineered cells according to any proceeding Item, wherein the genome editing complex is an RNA-guided nuclease.

772. The population of engineered cells according to any proceeding Item, wherein the RNA-guided nuclease comprises a Cas nuclease and a guide RNA (CRISPR-Cas combination).

773. The population of engineered cells according to any proceeding Item, wherein the CRISPR-Cas combination is a ribonucleoprotein (RNP) complex comprising the gRNA and the Cas nuclease.

774. The population of engineered cells according to any proceeding Item, wherein the Cas nuclease is a Type II or Type V Cas protein.

775. The population of engineered cells according to any proceeding Item, wherein the Cas nuclease is Cas3, Cas4, Cas5, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr5, Cse1, Cse2, Csf1, Csm2, Csn2, Csx10, Csx11, Csy1, Csy2, Csy3, or Mad7.

776. The population of engineered cells according to any proceeding Item, wherein the one or more genetic modifications are made at a modification site.

777. The population of engineered cells according to any proceeding Item, wherein the modification site is 25 nucleotides or less from a protospacer adjacent motif (PAM) sequence, wherein the PAM sequence is ngg, nag, ngrrt, ngrrn, nnnngatt, nnnnryac, nnagaaw, naaaac, tttv, ttn, attn, tttn, gttn, or yttn and wherein: (ii) r=a or g, (iii) y=c or t, (iv) w=a or t, (v) v=a or c or g, and (vi) n=a, c, t, org.

778. The population of engineered cells according to any proceeding Item, wherein the one or more genetic modifications are introduced using homology-directed repair (HDR)-mediated modification using SpCas9 and the PAM is ngg or nag, wherein n=a, c, t, or g.

779. The population of engineered cells according to any proceeding Item, wherein the one or more genetic modifications are introduced using homology-directed repair (HDR)-mediated modification using SaCas9 and the PAM is ngrrt or ngrrn, wherein: (vii) r=a or g, and (viii) n=a, c, t, or g.

780. The population of engineered cells according to any proceeding Item, wherein the one or more genetic modifications are introduced using homology-directed repair (HDR)-mediated modification using NmeCas9 and the PAM is nnnngatt, wherein n=a, c, t, or g.

781. The population of engineered cells according to any proceeding Item, wherein the one or more genetic modifications are introduced using homology-directed repair (HDR)-mediated modification using CjCas9 and the PAM is nnnnryac, wherein: (ix) r=a or g, (x) y=c or t, and (xi) n=a, c, t, or g.

782. The population of engineered cells according to any proceeding Item, wherein the one or more genetic modifications are introduced using homology-directed repair (HDR)-mediated modification using StCas9 and the PAM is nnagaaw wherein: (xii) w=a or t, and (xiii) n=a, c, t, or g.

783. The population of engineered cells according to any proceeding Item, wherein the one or more genetic modifications are introduced using homology-directed repair (HDR)-mediated modification using TdCas9 and the PAM is naaaac, wherein n=a, c, t, or g.

784. The population of engineered cells according to any proceeding Item, wherein the one or more genetic modifications are introduced using homology-directed repair (HDR)-mediated modification using LbCas12a and the PAM is tttv, wherein v=a or c or g.

785. The population of engineered cells according to any proceeding Item, wherein the one or more genetic modifications are introduced using homology-directed repair (HDR)-mediated modification using AsCas12a and the PAM is tttv, wherein v=a or c or g.

786. The population of engineered cells according to any proceeding Item, wherein the one or more genetic modifications are introduced using homology-directed repair (HDR)-mediated modification using AacCas12b and the PAM is ttn, wherein n=a, c, t, or g.

787. The population of engineered cells according to any proceeding Item, wherein the one or more genetic modifications are introduced using homology-directed repair (HDR)-mediated modification using BhCas12b and the PAM is attn., tttn, or gttn, wherein n=a, c, t, or g.

788. The population of engineered cells according to any proceeding Item, wherein the one or more genetic modifications are introduced using homology-directed repair (HDR)-mediated modification using MAD7 (ErCasl2a) and the PAM is yttn, wherein: (xiv) y=c or t, and (xv) n=a, c, t, or g.

789. The population of engineered cells according to any proceeding Item, the one or more genetic modifications are introduced by inducing an insertion or a deletion in the gene using a gene editing system ex vivo from a donor subject.

790. The population of engineered cells according to any proceeding Item, wherein the engineered cells of the population comprise one or more exogenous polynucleotides that encode one or more tolerogenic factors.

791. The population of engineered cells according to any proceeding Item, wherein the one or more tolerogenic factors comprise A20/TNFAIP3, C1-Inhibitor, CCL21, CCL22, CD16, CD16 Fc receptor, CD24, CD27, CD35, CD39, CD46, CD47, CD52, CD55, CD59, CD64, CD200, CR1, CTLA4-Ig, DUX4, FasL, H2-M3, HLA-C, HLA-E, HLA-E heavy chain, HLA-F, HLA-G, IDO1, IL-10, IL15-RF, IL-35, IL-39, MANF, Mfge8, PD-L1, Serpinb9, CCL21, CCL22, B2M-HLA-E, C1 inhibitor, CR1, or a combination thereof.

792. The population of engineered cells according to any proceeding Item, wherein the engineered cells of the population comprise an exogenous polynucleotides that encode CD24.

793. The population of engineered cells according to any proceeding Item, wherein the engineered cells of the population comprise an exogenous polynucleotides that encode CD47.

794. The population of engineered cells according to any proceeding Item, wherein the engineered cells of the population comprise an exogenous polynucleotides that encode CD52.

795. The population of engineered cells according to any proceeding Item, wherein the engineered cells of the population comprise an exogenous polynucleotides that encode CD70.

796. The population of engineered cells according to any proceeding Item, wherein the engineered cells of the population express A20/TNFAIP3, C1-Inhibitor, CCL21, CCL22, CD16, CD16 Fc receptor, CD24, CD27, CD35, CD39, CD46, CD47, CD52, CD55, CD59, CD64, CD200, CR1, CTLA4-Ig, DUX4, FasL, H2-M3, HLA-C, HLA-E, HLA-E heavy chain, HLA-F, HLA-G, IDO1, IL-10, IL15-RF, IL-35, IL-39, MANF, Mfge8, PD-L1, Serpinb9, CCL21, CCL22, B2M-HLA-E, C1 inhibitor, CR1, and any combination thereof from one or more exogenous polynucleotides.

797. The population of engineered cells according to any proceeding Item, wherein the engineered cells of the population express CD47, HLA-E, and PD-L1 from one or more exogenous polynucleotides.

798. The population of engineered cells according to any proceeding Item, wherein the engineered cells of the population express CD24 from an exogenous polynucleotide.

799. The population of engineered cells according to any proceeding Item, wherein the engineered cells of the population express CD47 from an exogenous polynucleotide.

800. The population of engineered cells according to any proceeding Item, wherein the engineered cells of the population express CD52 from an exogenous polynucleotide.

801. The population of engineered cells according to any proceeding Item, wherein the engineered cells of the population express CD70 from an exogenous polynucleotide.

802. The population of engineered cells according to any proceeding Item, wherein the engineered cells of the population express CD47 from one or more exogenous polynucleotides.

803. The population of engineered cells according to any proceeding Item, wherein one or more exogenous polynucleotides encoding one or more tolerogenic factors and/or one or more exogenous polynucleotides encoding one or more CARs are introduced at a safe harbor locus, a target locus, an RHD locus, a B2M locus, a CIITA locus, a TRAC locus, or a TRB locus.

804. The population of engineered cells according to any proceeding Item, wherein the safe harbor locus is a CCR5 locus, a PPP1R12C locus, a CLYBL locus, or a Rosa locus.

805. The population of engineered cells according to any proceeding Item, wherein the target locus is a CXCR4 locus, an ALB locus, a SHS231 locus, an F3 (CD142) locus, a MICA locus, a MICB locus, a LRP1 (CD91) locus, a HMGB1 locus, an ABO locus, a FUT1 locus, or a KDM5D locus.

806. The population of engineered cells according to any proceeding Item, wherein one or more exogenous polynucleotides encoding one or more tolerogenic factors and/or one or more exogenous polynucleotides encoding one or more CARs are introduced into the first engineered cell using a gene therapy vector or a transposase system.

807. The population of engineered cells according to any proceeding Item, wherein the transposase system comprises a transposase, a PiggyBac transposon, a Sleeping Beauty (SB11) transposon, a Mos1 transposon, or a Tol2 transposon.

808. The population of engineered cells according to any proceeding Item, wherein the gene therapy vector is a retrovirus or a fusosome.

809. The population of engineered cells according to any proceeding Item, wherein one or more exogenous polynucleotides encoding one or more tolerogenic factors and/or one or more exogenous polynucleotides encoding one or more CARs are encoded by a polycistronic vector.

810. The population of engineered cells according to any proceeding Item, wherein the polycistronic vector is a bicistronic vector comprising one exogenous polynucleotide encoding a tolerogenic factor and one exogenous polynucleotide encoding one or more CARs.

811. The population of engineered cells according to any proceeding Item, wherein the engineered cells of the population comprise reduced expression of a HLA-I complex or reduced expression of a HLA-II complex relative to an unaltered or unmodified wild-type or control cell.

812. The population of engineered cells according to any proceeding Item, wherein the engineered cells of the population comprise reduced expression of B2M or reduced expression of CIITA relative to an unaltered or unmodified wild-type or control cell.

813. The population of engineered cells according to any proceeding Item, wherein the engineered cells of the population comprise (i) reduced expression of a HLA-I complex and (ii) reduced expression of a HLA-II complex relative to an unaltered or unmodified wild-type or control cell.

814. The population of engineered cells according to any proceeding Item, wherein the engineered cells of the population comprise (i) reduced expression of B2M and (ii) reduced expression of CIITA relative to an unaltered or unmodified wild-type or control cell.

815. The population of engineered cells according to any proceeding Item, wherein engineered cells (e.g., engineered CAR-T cells) of the first, second, and/or third population comprise (i) reduced expression of a HLA-I complex or reduced expression of a HLA-II complex relative to an unaltered or unmodified wild-type or control cell, and (ii) increased expression of CD47 relative to an unaltered or unmodified wild-type or control cell.

816. The population of engineered cells according to any proceeding Item, wherein engineered cells (e.g., engineered CAR-T cells) of the first, second, and/or third population comprise (i) reduced expression of B2M or reduced expression of CIITA relative to an unaltered or unmodified wild-type or control cell, and (ii) increased expression of CD47 relative to an unaltered or unmodified wild-type or control cell.

817. The population of engineered cells according to any proceeding Item, wherein engineered cells (e.g., engineered CAR-T cells) of the first, second, and/or third population comprise (i) reduced expression of a HLA-I complex relative to an unaltered or unmodified wild-type or control cell,
(ii) reduced expression of a HLA-II complex relative to an unaltered or unmodified wild-type or control cell, and (iii) increased expression of CD47 relative to an unaltered or unmodified wild-type or control cell.

818. The population of engineered cells according to any proceeding Item, wherein engineered cells (e.g., engineered CAR-T cells) of the first, second, and/or third population comprise (i) reduced expression of B2M relative to an unaltered or unmodified wild-type or control cell, (ii) reduced expression of CIITA relative to an unaltered or unmodified wild-type or control cell, and (iii) increased expression of CD47 relative to an unaltered or unmodified wild-type or control cell.

819. The population of engineered cells according to any proceeding Item, wherein the engineered cells of the population comprise (i) reduced expression of a HLA-I complex or reduced expression of a HLA-II complex, and (ii) reduced expression of a TCR relative to an unaltered or unmodified wild-type or control cell.

820. The population of engineered cells according to any proceeding Item, wherein the engineered cells of the population comprise (i) reduced expression of B2M or reduced expression of CIITA, and (ii) reduced expression of a TRAC relative to an unaltered or unmodified wild-type or control cell.

821. The population of engineered cells according to any proceeding Item, wherein the engineered cells of the population comprise (i) reduced expression of a HLA-I complex, (ii) reduced expression of a HLA-II complex, and (iii) reduced expression of a TCR relative to an unaltered or unmodified wild-type or control cell.

822. The population of engineered cells according to any proceeding Item, wherein the engineered cells of the population comprise (i) reduced expression of B2M, (ii) reduced expression of CIITA, and (iii) reduced expression of a TRAC relative to an unaltered or unmodified wild-type or control cell.

823. The population of engineered cells according to any proceeding Item, wherein engineered cells (e.g., engineered CAR-T cells) of the first, second, and/or third population comprise (i) reduced expression of a HLA-I complex or reduced expression of a HLA-II complex relative to an unaltered or unmodified wild-type or control cell, (ii) reduced expression of a TRAC relative to an unaltered or unmodified wild-type or control cell, and (iii) increased expression of CD47 relative to an unaltered or unmodified wild-type or control cell.

824. The population of engineered cells according to any proceeding Item, wherein engineered cells (e.g., engineered CAR-T cells) of the first, second, and/or third population comprise (i) reduced expression of B2M or reduced expression of CIITA relative to an unaltered or unmodified wild-type or control cell, (ii) reduced expression of a TRAC relative to an unaltered or unmodified wild-type or control cell, and (ii) increased expression of CD47 relative to an unaltered or unmodified wild-type or control cell.

825. The population of engineered cells according to any proceeding Item, wherein engineered cells (e.g., engineered CAR-T cells) of the first, second, and/or third population comprise (i) reduced expression of a HLA-I complex relative to an unaltered or unmodified wild-type or control cell, (ii) reduced expression of a HLA-II complex relative to an unaltered or unmodified wild-type or control cell, (iii) reduced expression of a TRAC relative to an unaltered or unmodified wild-type or control cell, and (iv) increased expression of CD47 relative to an unaltered or unmodified wild-type or control cell.

826. The population of engineered cells according to any proceeding Item, wherein engineered cells (e.g., engineered CAR-T cells) of the first, second, and/or third population comprise (i) reduced expression of B2M relative to an unaltered or unmodified wild-type or control cell, (ii) reduced expression of CIITA relative to an unaltered or unmodified wild-type or control cell, (iii) reduced expression of a TRAC relative to an unaltered or unmodified wild-type or control cell, and (iv) increased expression of CD47 relative to an unaltered or unmodified wild-type or control cell.

827. The population of engineered cells according to any proceeding Item, wherein the engineered cells of the population comprise (i) reduced expression of B2M or reduced expression of CD52, and (ii) reduced expression of a TRAC relative to an unaltered or unmodified wild-type or control cell.

828. The population of engineered cells according to any proceeding Item, wherein the engineered cells of the population comprise (i) reduced expression of B2M, (ii) reduced expression of CD52, and (iii) reduced expression of a TRAC relative to an unaltered or unmodified wild-type or control cell.

829. The population of engineered cells according to any proceeding Item, wherein the engineered cells of the population comprise (i) reduced expression of B2M or reduced expression of CD70, and (ii) reduced expression of a TRAC relative to an unaltered or unmodified wild-type or control cell.

830. The population of engineered cells according to any proceeding Item, wherein the engineered cells of the population comprise (i) reduced expression of B2M, (ii) reduced expression of CD70, and (iii) reduced expression of a TRAC relative to an unaltered or unmodified wild-type or control cell.

831. The population of engineered cells according to any proceeding Item, wherein the engineered cells of the population comprise (i) reduced expression of PD-1, and (ii) reduced expression of a TRAC relative to an unaltered or unmodified wild-type or control cell.

832. The population of engineered cells according to any proceeding Item, wherein the engineered cells of the population is B2M^{indel/indel}, CIITA^{indel/indel}, TRAC^{indel/indel}, and/or TRAC^{indel/indel} cells.

### EXAMPLES

### Example 1: Dual Transduced CD19CAR×CD22CAR T Cells and CD22CAR T Cells Control Tumor Growth in Nsg Antigen Escape Tumor Model (NALM)

This Example describes an exemplary method for testing the efficacy of different CAR-T treatments in an animal system including tumor cells that acts as an antigen escape model. In particular, this Example demonstrates the successful testing of the efficacy of CD19 CAR-T cells, CD22 CAR-T cells, and CD19×CD22 CAR-T cells in an NSG mouse model inoculated with a 70%:30% mixture of Nalm6:Nalm6-CD19KO tumor cells as an antigen escape model.

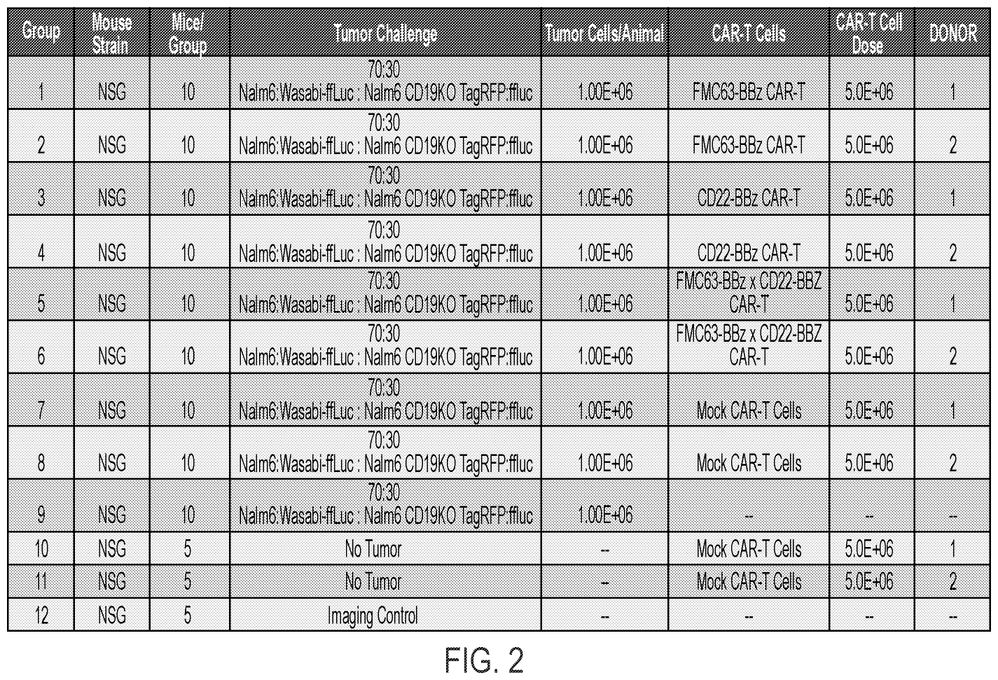

FIG. 1 shows the experimental timeline and experimental setup. Twelve groups of humanized mice were used for the experiment, as shown in FIG. 2.

On Day −4, a 70%:30% mixture of Nalm6 (Wasabi+):Nalm6-CD19KO (TagRFP+) tumor cells were introduced into Groups 1-9 of humanized mice at 1.0E+06 tumor cells/animal, as shown in FIG. 2. Groups 10-12 of humanized mice did not receive tumor cells. The Nalm6 (Wasabi+) tumor cells and Nalm6-CD19KO (TagRFP+) tumor cells used to generate a 70%:30% mixture each expressed luciferase, which was used for imaging.

On Day −3, all twelve groups of humanized mice were imaged.

On Day 0, experimental CAR-T cells or mock CAR-T cells were introduced into mice at 5.0E+06 CAR T cells/animal. The CAR-T cells used were allogenic and obtained from two different donors. As shown in FIG. 2, Groups 1-6 served as test groups. Group 1 mice had previously received tumor cells, and on Day 0, received CD19 CAR-T cells (FMC63-BBz CAR-T cells) from Donor 1. Group 2 mice had previously received tumor cells, and on Day 0, received CD19 CAR-T cells (FMC63-BBz CAR-T cells) from Donor 2. Group 3 mice had previously received tumor cells, and on Day 0, received CD22 CAR-T cells (CD22-BBz CAR-T cells) from Donor 1; Group 4 mice had previously received tumor cells, and on Day 0, received CD22 CAR-T cells (CD22-BBz CAR-T cells) from Donor 2. Group 5 mice had previously received tumor cells, and on Day 0, received CD19×CD22 CAR-T cells (FMC63-BBz×CD22-BBZ CAR-T cells) from Donor 1; Group 6 mice had previously received tumor cells, and on Day 0, received CD19×CD22 CAR-T cells (FMC63-BBz×CD22-BBZ CAR-T cells) from Donor 2. Group 7-12 mice were utilized as various controls. Group 7 mice had previously received tumor cells, and on Day 0, received Mock CAR-T cells from Donor 1; Group 8 mice had previously received tumor cells, and on Day 0, received Mock CAR-T cells from Donor 2. Group 9 mice had previously received tumor cells, and on Day 0, did not receive any CAR-T cells. Group 10 mice had not received tumor cells, but on Day 0, received Mock CAR-T cells from Donor 1; Group 11 mice also had not received tumor cells, but on Day 0, received Mock CAR-T cells from Donor 2. Group 12 mice received neither tumor cells nor Mock CAR-T cells and served as an imaging control.

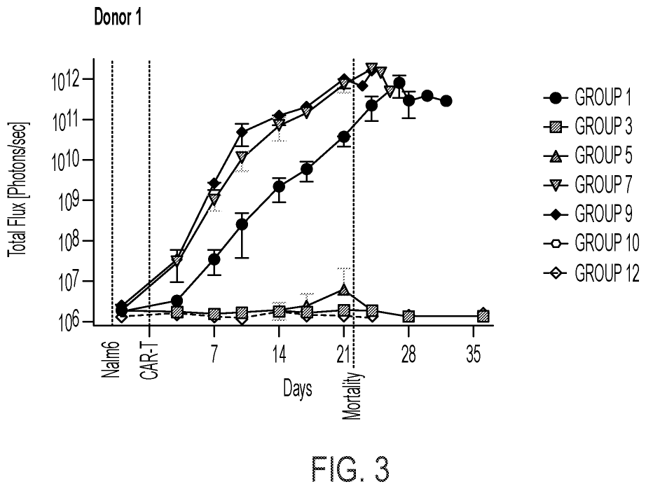

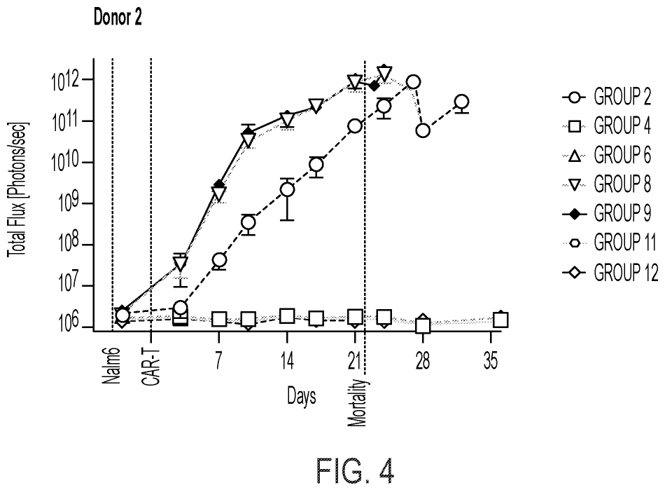

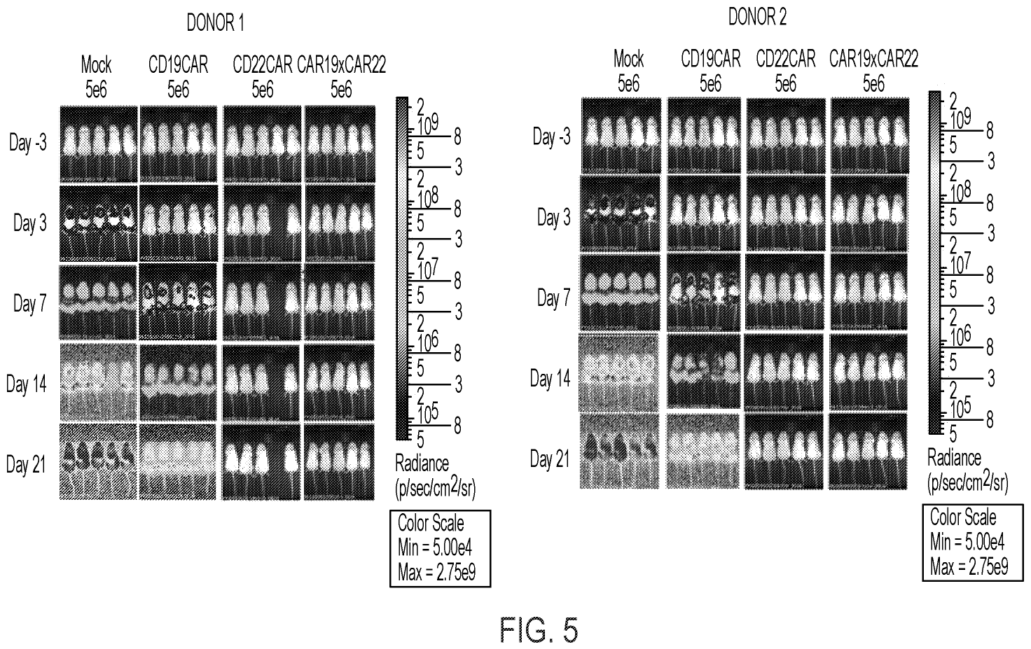

In vivo bioluminescent imaging was used to assess the presence of tumor cells in injected mice on Days 3, 7, 10, 14, 17, 21, 24, 28, and 36 (unless the mice had already been sacrificed due to tumor growth). Exemplary imaging is shown in FIG. 5 and exemplary line graphs of the total flux read from the bioluminescent imaging over time are shown in FIGS. 3 and 4.

Control data indicated that the experiment was working as expected. Mice in Groups 10 and 11, which had not received tumor cells, showed baseline levels of bioluminescence (FIGS. 3 and 4). Increasing bioluminescence was detected in Group 7 and 8 mice (FIGS. 3 and 4). The increasing bioluminescence indicated that the tumor cells these mice had received continued to expand over time and that the Mock CAR-T cells were not able to reduce the tumor burden in these mice.

As further shown in FIGS. 3 and 4, baseline levels of bioluminescence were detected from mice in Groups 3-6 through Day 36. The data obtained demonstrates that the CD22 CAR-T cells and CD19 CAR xCD22 CAR-T cells derived from two different T cell donors were able to effectively control NALM tumor growth through Day 36 in a CD19 antigen escape tumor model.

While baseline or low levels of bioluminescence were detected from mice in Group 1 and 2 on Day 3, the levels of bioluminescence then generally increased from Day 3 through Day 36. This data indicates that CD19 CAR-T cells only transiently controlled CD19KO tumor growth and the mice receiving CD19 CAR-T cells progressively succumbed to the tumor.

Collectively, the data demonstrated that CAR-T cells including a CAR directed to an antigen present on tumor cells, and with or without a CAR directed to a second antigen susceptible to antigen escape, have efficacy in reducing tumor burden, even when antigen escape has occurred. The data also confirms that efficacy could be achieved with (e.g., allogenic) CAR-T cells derived from different donors.

### Example 2: Dual Transduced CD19CAR×CD22CAR T Cells and CD22CAR T Cells Control Tumor Growth in NSG Antigen Escape Tumor Model (RAJI)

This Example describes an exemplary method for testing the efficacy of different CAR-T treatments in an animal system including tumor cells that acts as an antigen escape model. In particular, this Example demonstrates the successful testing of the efficacy of CD19 CAR-T cells, CD22 CAR-T cells, and CD19×CD22 CAR-T cells in an NSG mouse model inoculated with a 70%:30% mixture of RAJI:RAJI-CD19KO tumor cells as an antigen escape model.

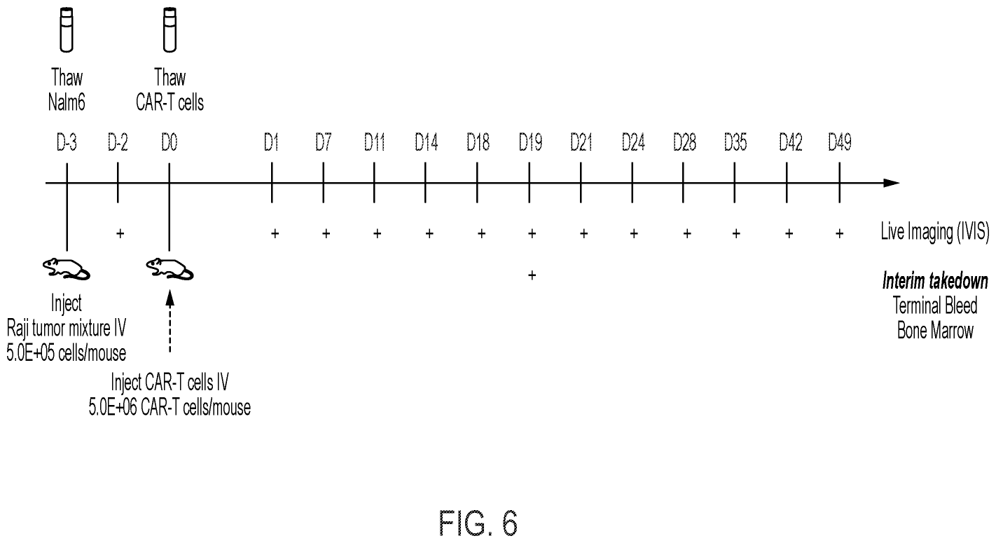

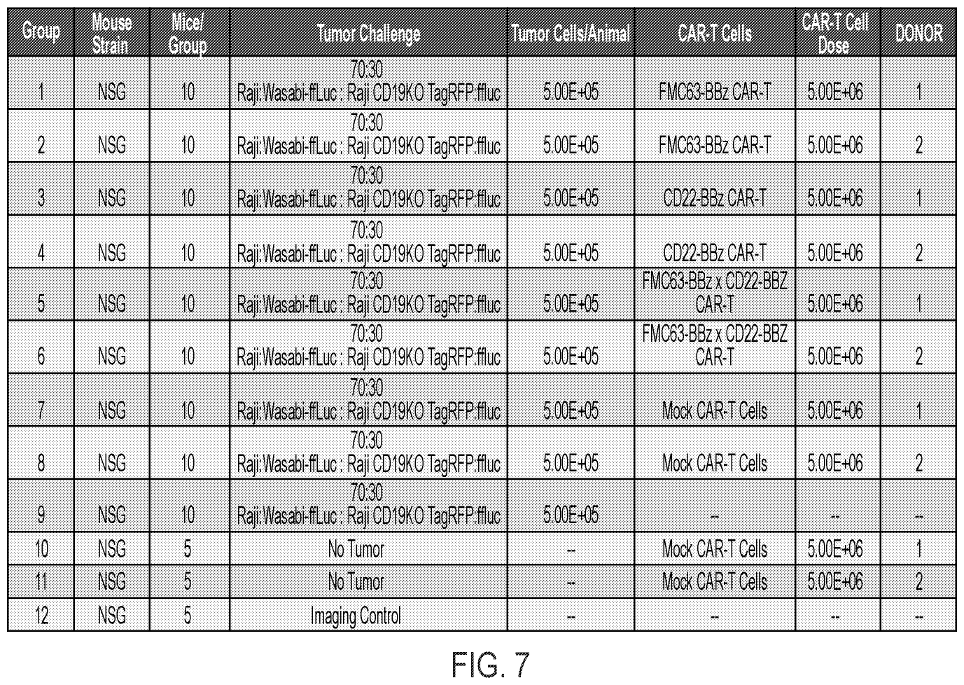

FIG. 6 shows the experimental timeline and experimental setup. Twelve groups of humanized mice were used for the experiment, as shown in FIG. 7.

On Day −3, a 70%:30% mixture of RAJI (Wasabi+):RAJI-CD19KO (TagRFP+) tumor cells were introduced into Groups 1-9 of humanized mice at 5.0E+05 tumor cells/animal, as shown in FIG. 7. Groups 10-12 of humanized mice did not receive tumor cells. The RAJI (Wasabi+):RAJI-CD19KO (TagRFP+) tumor cells used to generate a 70%:30% mixture each expressed luciferase, which was used for imaging.

On Day −2, all twelve groups of humanized mice were imaged.

On Day 0, experimental CAR-T cells or mock CAR-T cells were introduced into mice at 5.0E+06 CAR T cells/animal. The CAR-T cells used were allogenic and obtained from two different donors. As shown in FIG. 7, Groups 1-6 served as test groups. Group 1 mice had previously received tumor cells, and on Day 0, received CD19 CAR-T cells (FMC63-BBz CAR-T cells) from Donor 1. Group 2 mice had previously received tumor cells, and on Day 0, received CD19 CAR-T cells (FMC63-BBz CAR-T cells) from Donor 2. Group 3 mice had previously received tumor cells, and on Day 0, received CD22 CAR-T cells (CD22-BBz CAR-T cells) from Donor 1; Group 4 mice had previously received tumor cells, and on Day 0, received CD22 CAR-T cells (CD22-BBz CAR-T cells) from Donor 2. Group 5 mice had previously received tumor cells, and on Day 0, received CD19×CD22 CAR-T cells (FMC63-BBz×CD22-BBZ CAR-T cells) from Donor 1; Group 6 mice had previously received tumor cells, and on Day 0, received CD19×CD22 CAR-T cells (FMC63-BBz×CD22-BBZ CAR-T cells) from Donor 2. Group 7-12 mice were utilized as various controls. Group 7 mice had previously received tumor cells, and on Day 0, received Mock CAR-T cells from Donor 1; Group 8 mice had previously received tumor cells, and on Day 0, received Mock CAR-T cells from Donor 2. Group 9 mice had previously received tumor cells, and on Day 0, did not receive any CAR-T cells. Group 10 mice had not received tumor cells, but on Day 0, received Mock CAR-T cells from Donor 1; Group 11 mice also had not received tumor cells, but on Day 0, received Mock CAR-T cells from Donor 2. Group 12 mice received neither tumor cells nor Mock CAR-T cells and served as an imaging control.

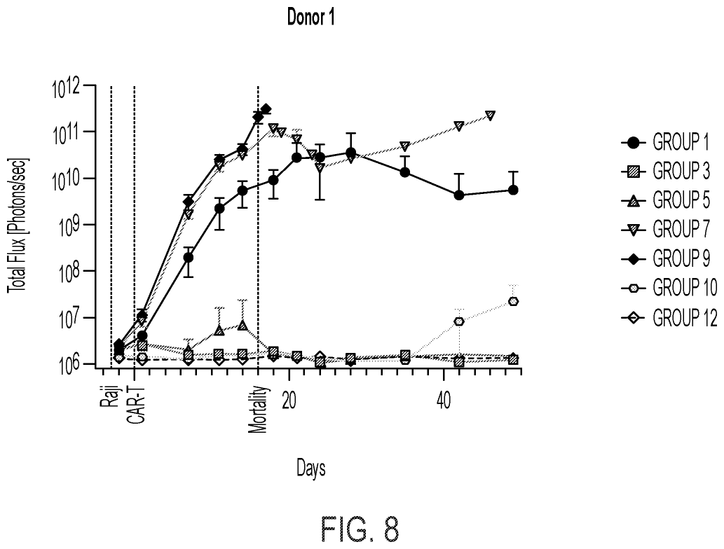

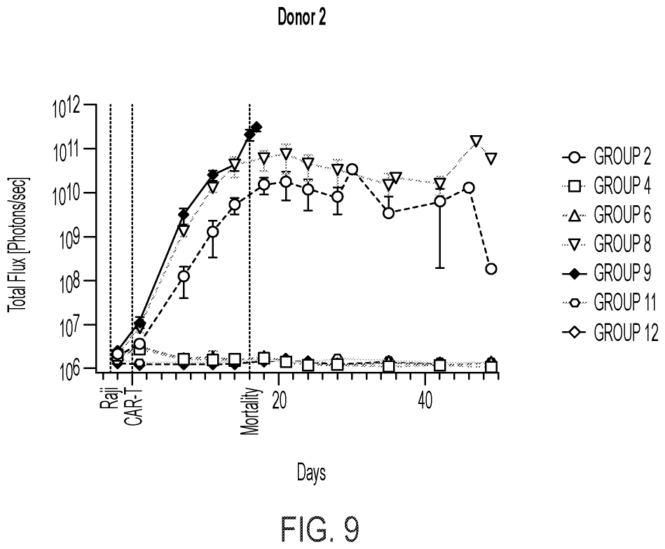

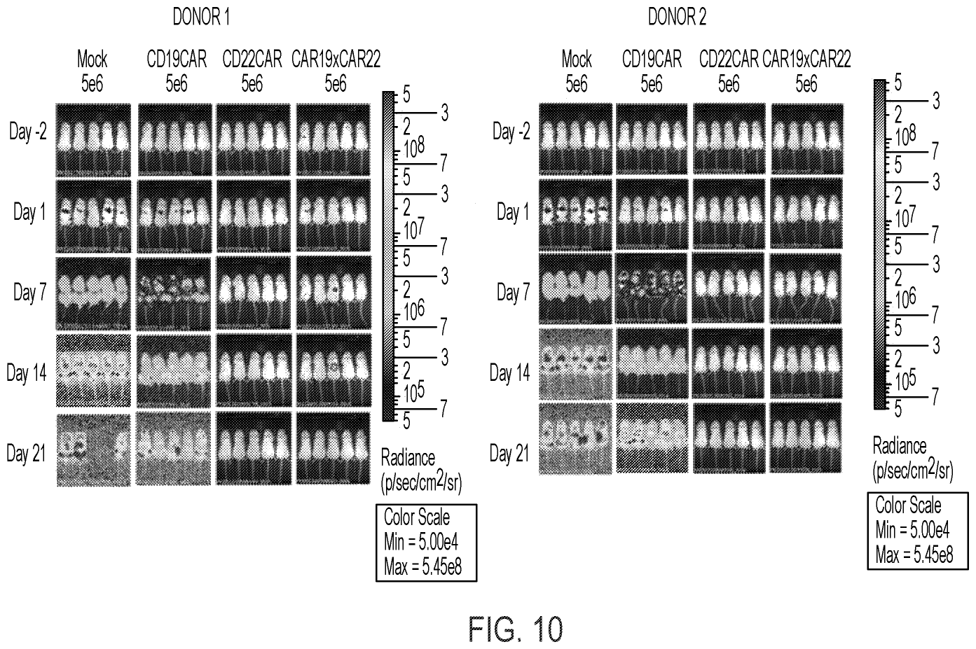

In vivo bioluminescent imaging was used to assess the presence of tumor cells in injected mice on Days 1, 7, 11, 14, 18, 19, 21, 24, 28, 35, 42, and 49 (unless the mice had already been sacrificed due to tumor growth). Exemplary imaging is shown in FIG. 10 and exemplary line graphs of the total flux read from the bioluminescent imaging over time are shown in FIGS. 8 and 9.

Control data indicated that the experiment was working as expected. Mice in Groups 10 and 11, which had not received tumor cells, showed baseline levels of bioluminescence (FIGS. 8 and 9). Increasing bioluminescence was detected in Group 7 and 8 mice (FIGS. 8 and 9). The increasing bioluminescence indicated that the tumor cells these mice had received continued to expand over time and that the Mock CAR-T cells were not able to reduce the tumor burden in these mice.

As further shown in FIGS. 8 and 9, baseline levels of bioluminescence were detected from mice in Groups 3-6 through Day 36. The data obtained demonstrates that the CD22 CAR-T cells and CD19 CAR×CD22 CAR-T cells derived from two different T cell donors were able to effectively control RAJI tumor growth through Day 49 in a CD19 antigen escape tumor model.

While baseline or low levels of bioluminescence were detected from mice in Group 1 and 2 on Day 1, the levels of bioluminescence then generally increased from Day 1 through Day 49. This data indicates that CD19 CAR-T cells only transiently controlled CD19KO tumor growth and the mice receiving CD19 CAR-T cells progressively succumbed to the tumor.

Collectively, the data demonstrated that CAR-T cells including a CAR directed to an antigen present on tumor cells, and with or without a CAR directed to a second antigen susceptible to antigen escape, have efficacy in reducing tumor burden, even when antigen escape has occurred. Comparing the data in this Example and Example 1 shows that similar results were obtained using two different tumor cell types, suggesting that the efficacy of the CAR-T cells is not limited to a particular tumor type. The data also confirms that efficacy could be achieved with (e.g., allogenic) CAR-T cells derived from different donors.

### Example 3: Dual Transduced (CD19 CAR and CD22 CAR) CAR-T Cells Control Tumor Growth Better than 50:50 Mix of CD19 CAR-T Cells and CD22 CAR-T Cells

This Example describes an exemplary method for testing the efficacy of different CAR-T treatments in an animal system including tumor cells that acts as an antigen escape model. In particular, this Example demonstrates the successful testing the antitumor activity of dual transduced (CD19 CAR and CD22 CAR) CAR-T cells (which includes CD19 CAR-T, CD22 CAR-T, and CD19×CD22 CAR-T) or dual transduced and sorted CAR-T cells (which includes only CD19×CD22 CAR-T), versus the antitumor activity of a combined product of single transduced CD19 CAR-T cells and single transduced and CD22 CAR-T cells.

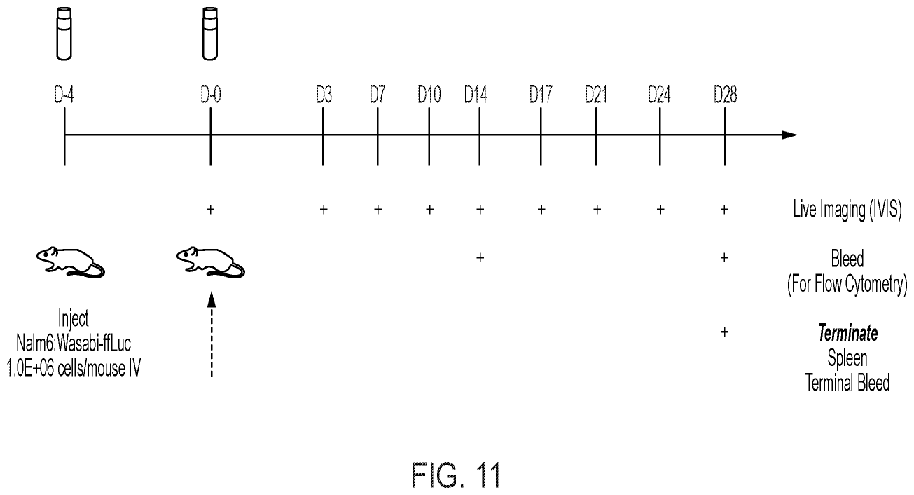

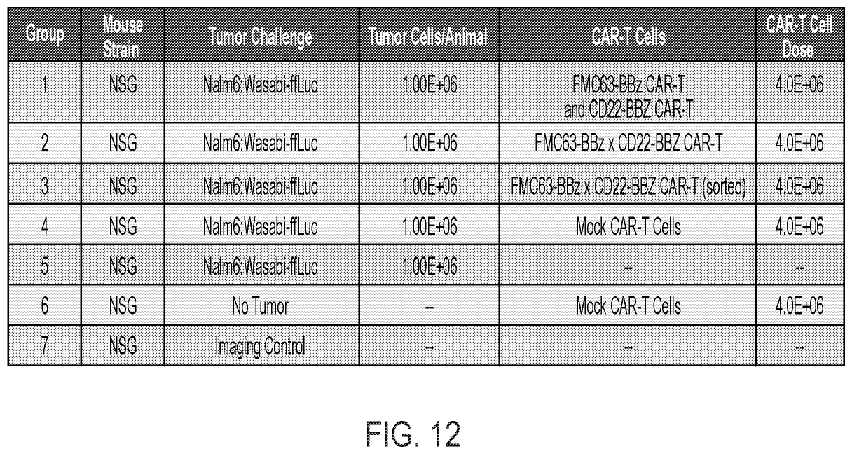

FIG. 11 shows the experimental timeline and experimental setup. Seven groups of humanized mice were used for the experiment, as shown in FIG. 12.

On Day −4, Nalm6 tumor cells were introduced into Groups 1-5 of humanized mice at 1.0E+06 tumor cells/animal, as shown in FIG. 12. Groups 6 and 7 did not receive tumor cells. The Nalm6 tumor cells expressed luciferase, which was used for imaging.

On Day 0, experimental CAR-T cells or mock CAR-T cells were introduced into mice at varied doses, e.g., 4.0E+06, 2.0E+06, 1.0E+06, and 4.0E+05 CAR T cells/animal. The CAR-T cells used were allogenic and obtained from two different donors. As shown in FIG. 12, Groups 1-3 served as test groups. Group 1 mice had previously received tumor cells, and on Day 0, received a combined product of single transduced CD19 CAR-T cells and single transduced and CD22 CAR-T cells. Group 2 mice had previously received tumor cells, and on Day 0, received dual transduced CAR-T cells (which include CD19 CAR-T, CD22 CAR-T, and CD19×CD22 CAR-T). Group 3 mice had previously received tumor cells, and on Day 0, received dual transduced and sorted CAR-T cells (which include only CD19×CD22 CAR-T). Group 4-6 mice were utilized as various controls. Group 4 mice had previously received tumor cells, and on Day 0, received Mock CAR-T cells. Group 5 mice had previously received tumor cells, and on Day 0, did not receive any CAR-T cells. Group 6 mice had not received tumor cells, but on Day 0, received Mock CAR-T cells. Group 7 mice received neither tumor cells nor Mock CAR-T cells and served as an imaging control.

In vivo bioluminescent imaging was used to assess the presence of tumor cells in injected mice on Days 10, 3, 7, 10, 14, 17, 21, 24, and 28 (unless the mice had already been sacrificed due to tumor growth). Exemplary line graphs of the total flux read from the bioluminescent imaging over time are shown in FIG. 13. Data shown is for mice that received CAR-T cells at a dose of 4.0E+06 CAR T cells/animal.

Once again, control data indicated that the experiment was working as expected. Mice in Groups 6 and 7, which had not received tumor cells, showed baseline levels of bioluminescence (FIG. 13). Increasing bioluminescence was detected in Group 4 and 5 mice (FIG. 13). The increasing bioluminescence indicated that the tumor cells these mice had received continued to expand over time and that the Mock CAR-T cells were not able to reduce the tumor burden in these mice.

As shown in FIG. 13, the bioluminescence data demonstrates that dual transduced CAR-T cells (which include CD19 CAR-T, CD22 CAR-T, and CD19×CD22 CAR-T) and dual transduced and sorted (which include only CD19×CD22 CAR-T) controlled tumor levels more efficiently than a combined product of single transduced CD19 CAR-T cells and single transduced and CD22 CAR-T cells. This data indicates that a population of cells comprising CAR-T cells with two or more CARs may be more successful in eliminating tumor cells, in particular tumor cells prone to antigen evasion, than CAR-T cells comprising a single CAR or mixtures of CAR-T cells that each comprise a single CAR. Further, this data indicates that, where particular tumor cells are prone to antigen evasion, a population of cells comprising a mixture of both single CAR cells and dual CAR cells (e.g., a mixture that includes CD19 CAR-T, CD22 CAR-T, and CD19×CD22 CAR-T) may be successful in eliminating tumor cells.

### EQUIVALENTS

Many modifications and variations of this application can be made without departing from its spirit and scope, as will be apparent to those skilled in the art. The specific embodiments and examples described herein are offered by way of example only, and the application is to be limited only by the terms of the appended claims, along with the full scope of equivalents to which the claims are entitled.

All headings and section designations are used for clarity and reference purposes only and are not to be considered limiting in any way. For example, those of skill in the art will appreciate the usefulness of combining various embodiments from different headings and sections as appropriate according to the spirit and scope of the technology described herein.

All references cited herein are hereby incorporated by reference herein in their entireties and for all purposes to the same extent as if each individual publication or patent or patent application was specifically and individually indicated to be incorporated by reference in its entirety for all purposes.

1. A method of treating a disease or disorder in a patient comprising administering a therapeutic agent to the patient,
wherein the therapeutic agent comprises a first population of engineered CAR-T cells and a second population of engineered CAR-T cells,
wherein the first population of engineered CAR-T cells comprises one or more chimeric antigen receptors (CARs), wherein at least one CAR of the first population of engineered CAR-T cells (i) is directed to a first therapeutic target and (ii) comprises a first antigen binding domain,
wherein the second population of engineered CAR-T cells comprises one or more CARs, wherein at least one CAR of the second population of engineered CAR-T cell (i) is directed to a second therapeutic target and (ii) comprises a second antigen binding domain, and
wherein the first therapeutic target and the second therapeutic target are different, and
wherein the patient has previously been administered one or more targeted therapies directed to the second therapeutic target.

2. The method of claim 1, wherein the therapeutic agent further comprises a third population of engineered CAR-T cells, wherein the third population of engineered CAR-T cells comprises two or more CARs,
wherein at least one CAR of the third population of engineered CAR-T cell (i) is directed to the first therapeutic target and (ii) comprises the first antigen binding domain, and
wherein at least one CAR of the third population of engineered CAR-T cell (i) is directed to the second therapeutic target, and (ii) comprises the second antigen binding domain.

3. The method of claim 1, wherein the patient has not previously received a therapy directed to the first therapeutic target.

4. The method of claim 1, wherein the patient is at risk of antigen evasion and/or the disease or disorder is characterized by antigen evasion.

5. (canceled)

6. The method of claim 1, wherein the disease or disorder is cancer.

7-8. (canceled)

9. The method of claim 6, wherein the cancer is a B cell malignancy or a B cell lymphoma.

10. The method of claim 1, wherein the first and/or second therapeutic target is an antigen chosen from CD22, CD20, CD19, BCMA, GPRC5D, CD38, CD70, CD79b, HER2, IL13Ra2, and MU.

11. The method of claim 10, wherein the first therapeutic target is a CD22 antigen or a CD20 antigen and the second therapeutic target is a CD19 antigen.

12-24. (canceled)

25. The method of claim 1, wherein the first and/or second population of engineered CAR-T cells comprise one or more genetic modifications that, relative to an unaltered or unmodified wild-type or control cell:
(i) reduce expression of one or more major histocompatibility complex class I human leukocyte antigen (HLA-I) molecules or one or more HLA-I associated molecules; and/or
(ii) reduce expression of one or more major histocompatibility complex class II human leukocyte antigen (HLA-II) molecules or one or more HLA-II associated molecules.

26. (canceled)

27. The method of claim 25, wherein the one or more HLA-I associated molecules comprise β-2 microglobulin (B2M) and the one or more HLA-II associated molecules comprise CIITA.

28-30. (canceled)

31. The method of claim 1, wherein the first and/or second population of engineered CAR-T cells comprise one or more genetic modifications that reduce expression of a T cell receptor (TCR) relative to an unaltered or unmodified wild-type or control cell.

32. The method of claim 1, wherein the first and/or second population of engineered CAR-T cells do not express TCR-alpha (TRAC) and/or TCR-beta (TRBC).

33. The method of claim 1, wherein the first and/or second population of engineered CAR-T cells comprise one or more exogenous polynucleotides that encode one or more tolerogenic factors chosen from A20/TNFAIP3, C1-Inhibitor, CCL21, CCL22, CD16, CD16 Fc receptor, CD24, CD27, CD35, CD39, CD46, CD47, CD52, CD55, CD59, CD64, CD200, CR1, CTLA4-Ig, DUX4, FasL, H2-M3, HLA-C, HLA-E, HLA-E heavy chain, HLA-F, HLA-G, IDO1, IL-10, IL15-RF, IL-35, IL-39, MANF, Mfge8, PD-L1, Serpinb9, CCL21, CCL22, B2M-HLA-E, C1 inhibitor, CR1, or a combination thereof.

34. (canceled)

35. The method of claim 33, wherein the first and/or second population of engineered CAR-T cells comprise an exogenous polynucleotide that encodes CD47.

36-37. (canceled)

38. The method of claim 2, wherein the third population of engineered CAR-T cells comprises one or more genetic modifications that, relative to an unaltered or unmodified wild-type or control cell:
(i) reduce expression of one or more major histocompatibility complex class I human leukocyte antigen (HLA-I) molecules or one or more HLA-I associated molecules; and/or
(ii) reduce expression of one or more major histocompatibility complex class II human leukocyte antigen (HLA-II) molecules or one or more HLA-II associated molecules.

39. (canceled)

40. The method of claim 38, wherein the one or more HLA-I associated molecules comprise β-2 microglobulin (B2M) and the one or more HLA-II associated molecules comprise CIITA.

41-43. (canceled)

44. The method of claim 2, wherein the third population of engineered CAR-T cells comprises one or more genetic modifications that reduce expression of a T cell receptor (TCR) relative to an unaltered or unmodified wild-type or control cell.

45. The method of claim 2, wherein the third population of engineered CAR-T cells does not express TCR-alpha (TRACI and/or TCR-beta (TRBC).

46. The method of claim 2, wherein the third population of engineered CAR-T cells comprises one or more exogenous polynucleotides that encode one or more tolerogenic factors chosen from A20/TNFAIP3, C1-Inhibitor, CCL21, CCL22, CD16, CD16 Fc receptor, CD24, CD27, CD35, CD39, CD46, CD47, CD52, CD55, CD59, CD64, CD200, CR1, CTLA4-Ig, DUX4, FasL, H2-M3, HLA-C, HLA-E, HLA-E heavy chain, HLA-F, HLA-G, IDO1, IL-10, IL15-RF, IL-35, IL-39, MANF, Mfge8, PD-L1, Serpinb9, CCL21, CCL22, B2M-HLA-E, C1 inhibitor, CR1, or a combination thereof.

47. (canceled)

48. The method of claim 46, wherein the third population of engineered CAR-T cells comprises an exogenous polynucleotide that encodes CD47.

49. (canceled)

50. A method of treating a patient with or at risk of a disease or disorder associated with antigen evasion the method comprising administering a population of engineered CAR-T cells to the patient,
wherein patient that has previously been administered one or more targeted therapies directed to a second therapeutic target,
wherein the population of engineered CAR-T cells comprises one or more chimeric antigen receptors (CARs), wherein at least one CAR is directed to the first therapeutic target,
wherein the first therapeutic target and the second therapeutic target are different.

51. (canceled)
